(19) United States
(12) Patent Application Publication
(10) Pub. No.: US 2026/0337637 A1
(43) Pub. Date: Oct. 29, 2026

(54) GENE EDITING SYSTEMS AND COMPOSITIONS FOR TREATMENT OF HEMOGLOBINOPATHIES AND METHODS OF USING THE SAME

(71) Applicant: ReNAgade Therapeutics Management Inc., Watertown, MA (US)

(72) Inventors: Aisha Aljanahi, Watertown, MA (US); Rajat Das, Watertown, MA (US); Daniel Egan, Watertown, MA (US); Giedrius Gasiunas, Vilnius (LT); Sierra Harken, Watertown, MA (US); Megan Hoban, Watertown, MA (US); Hari Jayaram, Watertown, MA (US); Muthusamy Jayaraman, Watertown, MA (US); Shinu John, Watertown, MA (US); Jacob Layer, Watertown, MA (US); Kourtney Mendello, Watertown, MA (US); Elisabeth Narayanan, Watertown, MA (US); Matthew Pandelakis, Watertown, MA (US); Vladimir Presnyak, Watertown, MA (US); Karolina Anna Kosakowska, Watertown, MA (US)

(21) Appl. No.: 19/485,005

(22) PCT Filed: May 16, 2024
(86) PCT No.: PCT/US2024/029749

§ 371 Date: Nov. 17, 2025

Related U.S. Application Data
(60) Provisional application No. 63/502,846, filed on May 17, 2023.
(60) Provisional application No. 63/584,014, filed on Sep. 20, 2023.
(60) Provisional application No. 63/593,764, filed on Oct. 27, 2023.
(60) Provisional application No. 63/557,134, filed on Feb. 23, 2024.
(60) Provisional application No. 63/570,053, filed on Mar. 26, 2024.
(60) Provisional application No. 63/638,004, filed on Apr. 24, 2024.

(51) Int. Cl. C12N 15/88; A61K 9/51; A61K 31/7105; A61K 38/46; C12N 9/22; C12N 15/11

(52) U.S. Cl. CPC C12N 15/88; A61K 9/5123; A61K 31/7105; A61K 38/465; C12N 9/226; C12N 15/11; C12N 2310/20

(57) ABSTRACT

The present disclosure describes improved LNP-based nucleobase editing systems and therapeutics for use in treating hemoglobinopathies, including sickle cell disease and beta-thalassemia. In particular, the disclosure describes improved LNPs, including novel and improved ionizable lipids for making LNPs, that enhance the targeted delivery of LNP-based nucleobase editing systems and therapeutics to red blood cell progenitor cells, enabling treatment of hemoglobinopathies, in vivo.

### RELATED APPLICATIONS

This application claims priority under 35 U.S.C. § 119(e) to U.S. Provisional Application Ser. No. 63/502,846, filed May 17, 2023, U.S. Provisional Application Ser. No. 63/584,014, filed Sep. 20, 2023, U.S. Provisional Application Ser. No. 63/593,764, filed Oct. 27, 2023, U.S. Provisional Application Ser. No. 63/557,134, filed Feb. 23, 2024, U.S. Provisional Application Ser. No. 63/570,053, filed Mar. 26, 2024, and U.S. Provisional Application Ser. No. 63/638,004, filed Apr. 24, 2024, each of which are incorporated herein by reference in their entireties.

The foregoing applications, and all documents cited therein and all documents cited or referenced herein, together with any manufacturer's instructions, descriptions, product specifications, and product sheets for any products mentioned herein or in any document incorporated by reference herein, are hereby incorporated herein by reference, and may be employed in the practice of the disclosed subject matter. More specifically, all referenced documents are incorporated by reference to the same extent as if each individual document was specifically and individually indicated to be incorporated by reference.

### TECHNICAL FIELD

The present disclosure generally relates to the field of in vivo gene editing as a treatment for hemoglobinopathies. More in particular, this disclosure relates to therapeutic compositions comprising a lipid nanoparticle (LNP) composition and a cargo gene editing system that is capable of delivery to a hematopoietic stem cell (HSC) in vivo, wherein the gene editing system, once delivered, is capable of installing one or more edits to a hemoglobin gene or regulatory region thereof in the genome of the HSC such that the hemoglobinopathy, such as sickle cell disease (SCD) or transfusion-dependent beta-thalassemia, is treated.

### BACKGROUND OF THE DISCLOSURE

Gene editing therapeutics have enormous potential for treating diseases, such as hemoglobinopathies, including under in vivo conditions, but there remains a need for more effective delivery of such systems to appropriate in situ sites within a patient in order to realize this potential. Hemoglobinopathies are a collection of disorders characterized by abnormal forms of hemoglobin and include, most notably, sickle cell disease (SCD) and beta-thalassemia (e.g., transfusion-dependent beta-thalassemia or TDT).

Hemoglobin (Hb) carries oxygen from the lungs to tissues in erythrocytes or red blood cells (RBCs). During prenatal development and until shortly after birth, hemoglobin is present in the form of fetal hemoglobin (HbF), a tetrameric protein composed of two alpha (α)-globin chains and two gamma (γ)-globin chains. HbF is largely replaced by adult hemoglobin (HbA), a tetrameric protein in which the γ-globin chains of HbF are replaced with beta (β)-globin chains, through a process known as globin switching. HbF is more efficient than IIbA at carrying oxygen. The average adult makes less than 1% HbF out of total hemoglobin. The α-hemoglobin gene (HBA) is located on chromosome 16, while the β-hemoglobin gene (HBB), A gamma (γ)-globin chain (HBG1, also known as gamma globin A), and G gamma (γ)-globin chain (HBG2, also known as gamma globin G) are located on chromosome 11 within the globin gene cluster (i.e., globin locus).

Certain hemoglobinopathies, such as SCD and beta-thalassemia, are caused by mutations in HBB. According to the Centers for Disease Control (CDC) as of the time of this filing, SCD affects millions of people throughout the world, including a projected approximately 100,000 Americans. SCD disproportionately affects those whose ancestors came from sub-Saharan Africa; Spanish-speaking regions in the Western Hemisphere (South America, the Caribbean, and Central America); Saudi Arabia; India; and Mediterranean countries such as Turkey, Greece, and Italy. The CDC reports that SCD occurs in the U.S. among about 1 out of every 365 Black or African-American births and among 1 out of every 16,300 Hispanic-American births. SCD is actually a collection of multiple specific forms of the disease which are characterized by different mutations in the HBB gene and with differing levels of severity. Beta-thalassemia, like SCD, is an inherited disease whereby one or mutations in a hemoglobin gene (e.g., mutations in the genes encoding the beta or alpha subunits, or other sites that disrupt hemoglobin levels) result in low hemoglobin levels ranging in levels of severity in a mutation-dependent way.

Treatments for hemoglobinopathies are limited and are focused generally on management of the symptoms commonly caused by these disorders, including management of pain (in the case of SCD, pain is due to the negative effects of the sickle-shaped red blood cells on blood circulation), infection, and anemia. In the case of SCD, there are at least four FDA-approved treatments to manage various complications from SCD and which have been shown to lengthen the lives of treated patients. However, there are no current curative treatments.

While at least one gene editing therapy—CASGEVY™ (exagamglogene autotemcel)—has been approved by the FDA in the U.S. for using in treating SCD and TDT by increasing the production of HbF, the treatment is an ex vivo therapy involving removing the patient's own blood stem cells, editing them outside of the body, and then engrafting the edited cells into the bone marrow. Such cell therapies have many recognized disadvantages, including myeloablative pre-conditioning, neutrophil engraftment failure, delayed platelet engraftment, and hypersensitivity reactions, among other adverse event risks. A gene editing therapy for treating and/or curing hemoglobinopathies in vivo directly in the patient in a safe and effective manner would be a significant advance in the art and would overcome the issues posed by ex vivo alternatives.

### SUMMARY

Described herein are compositions, methods, processes, and kits for the selection, design, preparation, manufacture, formulation, and/or use of LNP-based nucleobase editing systems and therapeutics comprising the same, for the treatment of hemoglobinopathies, including but not limited to sickle cell disease (SCD) and beta-thalassemia (e.g., transfusion-dependent beta-thalassema or TDT). In particular, described herein are compositions, methods, processes, and kits comprising nucleobase editing systems capable of executing one or more edits to the genome of a patient, in vivo, as part of an LNP formulation. Compositions include, but are not limited to, (a) nucleobase editing systems and/or components thereof, (b) DNA molecules encoding the nucleobase editing systems and/or components thereof, (c) RNA molecules encoding the nucleobase editing systems and/or components thereof, (d) RNA components of the nucleobase editing systems, such as guide RNAs or retron non-coding RNAs, (e) cells or cell lines stabling expressing or otherwise containing the nucleobase editing systems described herein, (f) edited cells or edited cell lines that have been edited by a nucleobase editing system described herein, (g) an LNP capable of delivery to an HSC, including an HSC in vivo, (h) ionizable lipids, structural lipids, phospholipids, and PEGylated lipids for preparing LNPs that are capable of delivery to HSCs, including HSCs in vivo, (i) LNPs encapsulated or formulated with a nucleobase editing system described herein, including DNA, RNA, and/or protein components of said nucleobase editing systems, and which LNPs are capable of delivery to HSCs, including HSCs in vivo, (j) cells or cell lines comprising the LNPs described herein, and (k) pharmaceutical compositions comprising the LNPs described herein which are formulated with a nucleobase editing system described herein. Methods described herein include but are not limited to (a) methods of making the lipid components of herein disclosed LNPs, (b) method of making the herein disclosed LNPs, (c) methods of making the nucleobase editing systems described herein, including methods of making any of the individual DNA, RNA, or protein components of the nucleobase editing systems described herein, (d) methods of formulating the LNPs described herein with the nucleobase editing systems disclosed herein, or with any component thereof, (e) methods of delivering the LNP or pharmaceutical composition comprising the LNPs to cell, tissue, or organs in vivo, (f) methods of administering the nucleobase editing systems to cells, tissues, or organs in vivo, and (g) methods of editing one or more HSCs, including HSCs in vivo, in a hemoglobin gene or regulatory region thereof using a nucleobase editing system described herein to treat, including permanently treat, a hemoglobinopathy, including SCD and TDT.

In one aspect, the present disclosure provides compositions, methods, processes, and kits for the selection, design, preparation, manufacture, formulation, and/or use of LNP-based CRISPR-type II gene editing systems and therapeutics comprising the same, for the treatment of hemoglobinopathies, including but not limited to sickle cell disease (SCD) and beta-thalassemia (e.g., transfusion-dependent beta-thalassema or TDT), by introducing one or more edits into a hemoglobin gene or a regulatory region thereof in an hematopoietic stem cell (HSC) in the body (i.e., in vivo) which results in increased production of fetal hemoglobin (HbF), thereby treating the hemoglobinopathy. Compositions include, but are not limited to, (a) CRISPR-type II gene editing systems and/or components thereof, (b) DNA molecules encoding the CRISPR-type II gene editing systems and/or components thereof, (c) RNA molecules encoding the CRISPR-type II gene editing systems and/or components thereof, (d) RNA components of the CRISPR-type II gene editing systems, such as guide RNAs, (e) cells or cell lines stabling expressing or otherwise containing the CRISPR-type II gene editing systems described herein, (f) edited cells or edited cell lines that have been edited by a CRISPR-type II gene editing system described herein, (g) an LNP capable of delivery to an HSC, including an HSC in vivo, (h) ionizable lipids, structural lipids, phospholipids, and PEGylated lipids for preparing LNPs that are capable of delivery to HSCs, including HSCs in vivo, (i) LNPs encapsulated or formulated with a CRISPR-type II gene editing system described herein, including DNA, RNA, and/or protein components of said CRISPR-type II gene editing systems, and which LNPs are capable of delivery to HSCs, including HSCs in vivo, (j) cells or cell lines comprising the LNPs described herein, and (k) pharmaceutical compositions comprising the LNPs described herein which are formulated with a CRISPR-type II gene editing system described herein. Methods described herein include but are not limited to (a) methods of making the lipid components of herein disclosed LNPs, (b) method of making the herein disclosed LNPs, (c) methods of making the CRISPR-type II gene editing systems described herein, including methods of making any of the individual DNA, RNA, or protein components of the CRISPR-type II gene editing systems described herein, (d) methods of formulating the LNPs described herein with the CRISPR-type II gene editing systems disclosed herein, or with any component thereof, (e) methods of delivering the LNP or pharmaceutical composition comprising the LNPs to cell, tissue, or organs in vivo, (f) methods of administering the CRISPR-type II gene editing systems to cells, tissues, or organs in vivo, and (g) methods of editing one or more HSCs, including HSCs in vivo, in a hemoglobin gene or regulatory region thereof using a CRISPR-type II gene editing system described herein to treat, including permanently treat, a hemoglobinopathy, including SCD and TDT.

In another aspect, the present disclosure provides compositions, methods, processes, and kits for the selection, design, preparation, manufacture, formulation, and/or use of LNP-based CRISPR-type V gene editing systems and therapeutics comprising the same, for the treatment of hemoglobinopathies, including but not limited to sickle cell disease (SCD) and beta-thalassemia (e.g., transfusion-dependent beta-thalassema or TDT), by introducing one or more edits into a hemoglobin gene or a regulatory region thereof in an hematopoietic stem cell (HSC) in the body (i.e., in vivo) which results in increased production of fetal hemoglobin (HbF), thereby treating the hemoglobinopathy. Compositions include, but are not limited to, (a) CRISPR-type V gene editing systems and/or components thereof, (b) DNA molecules encoding the CRISPR-type V gene editing systems and/or components thereof, (c) RNA molecules encoding the CRISPR-type V gene editing systems and/or components thereof, (d) RNA components of the CRISPR-type V gene editing systems, such as guide RNAs, (e) cells or cell lines stabling expressing or otherwise containing the CRISPR-type V gene editing systems described herein, (f) edited cells or edited cell lines that have been edited by a CRISPR-type V gene editing system described herein, (g) an LNP capable of delivery to an HSC, including an HSC in vivo, (h) ionizable lipids, structural lipids, phospholipids, and PEGylated lipids for preparing LNPs that are capable of delivery to HSCs, including HSCs in vivo, (i) LNPs encapsulated or formulated with a CRISPR-type V gene editing system described herein, including DNA, RNA, and/or protein components of said CRISPR-type V gene editing systems, and which LNPs are capable of delivery to HSCs, including HSCs in vivo, (j) cells or cell lines comprising the LNPs described herein, and (k) pharmaceutical compositions comprising the LNPs described herein which are formulated with a CRISPR-type V gene editing system described herein. Methods described herein include but are not limited to (a) methods of making the lipid components of herein disclosed LNPs, (b) method of making the herein disclosed LNPs, (c) methods of making the CRISPR-type V gene editing systems described herein, including methods of making any of the individual DNA, RNA, or protein components of the CRISPR-type V gene editing systems described herein, (d) methods of formulating the LNPs described herein with the CRISPR-type V gene editing systems disclosed herein, or with any component thereof, (e) methods of delivering the LNP or pharmaceutical composition comprising the LNPs to cell, tissue, or organs in vivo, (f) methods of administering the CRISPR-type V gene editing systems to cells, tissues, or organs in vivo, and (g) methods of editing one or more HSCs, including HSCs in vivo, in a hemoglobin gene or regulatory region thereof using a CRISPR-type V gene editing system described herein to treat, including permanently treat, a hemoglobinopathy, including SCD and TDT.

In another aspect, the present disclosure provides compositions, methods, processes, and kits for the selection, design, preparation, manufacture, formulation, and/or use of LNP-based retron gene editing systems and therapeutics comprising the same, for the treatment of hemoglobinopathies, including but not limited to sickle cell disease (SCD) and beta-thalassemia (e.g., transfusion-dependent beta-thalassema or TDT), by introducing one or more edits into a hemoglobin gene or a regulatory region thereof in an hematopoietic stem cell (HSC) in the body (i.e., in vivo) which results in increased production of fetal hemoglobin (HbF), thereby treating the hemoglobinopathy. Compositions include, but are not limited to, (a) retron gene editing systems and/or components thereof, (b) DNA molecules encoding the retron gene editing systems and/or components thereof, (c) RNA molecules encoding the retron gene editing systems and/or components thereof, (d) RNA components of the retron gene editing systems, such as guide RNAs, (e) cells or cell lines stabling expressing or otherwise containing the retron gene editing systems described herein, (f) edited cells or edited cell lines that have been edited by a retron gene editing system described herein, (g) an LNP capable of delivery to an HSC, including an HSC in vivo, (h) ionizable lipids, structural lipids, phospholipids, and PEGylated lipids for preparing LNPs that are capable of delivery to HSCs, including HSCs in vivo, (i) LNPs encapsulated or formulated with a retron gene editing system described herein, including DNA, RNA, and/or protein components of said retron gene editing systems, and which LNPs are capable of delivery to HSCs, including HSCs in vivo, (j) cells or cell lines comprising the LNPs described herein, and (k) pharmaceutical compositions comprising the LNPs described herein which are formulated with a retron gene editing system described herein. Methods described herein include but are not limited to (a) methods of making the lipid components of herein disclosed LNPs, (b) method of making the herein disclosed LNPs, (c) methods of making the retron gene editing systems described herein, including methods of making any of the individual DNA, RNA, or protein components of the retron gene editing systems described herein, (d) methods of formulating the LNPs described herein with the retron gene editing systems disclosed herein, or with any component thereof, (e) methods of delivering the LNP or pharmaceutical composition comprising the LNPs to cell, tissue, or organs in vivo, (f) methods of administering the retron gene editing systems to cells, tissues, or organs in vivo, and (g) methods of editing one or more HSCs, including HSCs in vivo, in a hemoglobin gene or regulatory region thereof using a retron gene editing system described herein to treat, including permanently treat, a hemoglobinopathy, including SCD and TDT.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 depicts a single lipid nanoparticle (LNP) comprising an RNA cargo, wherein the LNP comprises an ionizable lipid, a helper lipid, a cholesterol, a PEG-lipid, and an optional targeting ligand having specificity for a hematopoietic stem cell (HSC), and the RNA cargo comprise one or elements encoding (e.g., an mRNA encoding a CRISPR type II or type V nuclease) or constituting (e.g., a guide RNA) a gene editing system. In embodiments, the gene editing system is for installing one or more edits in a hemoglobin gene or regulatory region thereof (e.g., such as in accordance with the editing strategies described herein).

FIG. 2A outlines a therapeutic scheme for treating a subject having a hemoglobinopathy, such as sickle cell disease (SCD) or transfusion-dependent β-thalassemia (TDT), using an LNP composition of the disclosure which comprises in some embodiments a gene editing system, such as a CRISPR type II or type V nuclease, a base editor, or a prime editor, and a suitable guide RNA, wherein said gene editing system is capable of installing one or more edits that result in the production of normal hemoglobin, e.g., normal adult hemoglobin or normal fetal hemoglobin.

FIG. 2B provides an illustrative view of the information in FIG. 2A.

FIG. 2C is a schematic, adapted from Dev Comp Immunol. 2016 May; 58:18-29., depicting the lineages of hematopoietic cell types according to two models.

FIG. 3 is a diagram showing the HBB mutations (i.e., mutations in the gene encoding hemoglobin subunit beta) that result in β-hemoglobinopathies, including sickle cell disease (SCD) and β-thalassemia.

FIG. 4 illustrates various non-limiting strategies for utilizing gene editing to treat β-hemoglobinopathies. The upper panel depicts correction of E6V sickle mutation in IIBB exon 1 using a CRISPR-Cas9 system that cuts precisely at or proximal to the E6V sickle mutation site followed by contact with an HDR-template that installs the corrected sequence mediated by homology-directed repair. The lower panel, left side, depicts correction of the E6V sickle mutation in HBB exon 1 using a BE strategy (which converts a T to C using an ABE thereby converting a valine residue to an alanine residue (the so called Makassar variant of hemoglobin beta subunit which functions normally). The lower panel, right side, depicts correction of the E6V sickle mutation in HBB exon 1 using a PE strategy (which rewrites the region containing the edit thereby restoring the wildtype sequence and production of a normal hemoglobin beta subunit).

FIG. 5 illustrates four strategies for reactivating γ-globin (aka HBG1/HBG2 or hemoglobin subunit gamma 1 and 2) production. In the first strategy (top), an HPFH deletion of the genes encoding HBD and HBB is introduced, thereby leading to reactivation of HBG1 and HBG2. This deletion emulates an HPFH (hereditary persistence of fetal hemoglobin mutation—which is a naturally occurring mutation in subjects which results in increased fetal hemoglobin production). In the second strategy (middle), HPFH mutations are introduced by editing into the HBG1/2 promoters which activates fetal hemoglobin production—again, emulating the natural effect of these mutations. This includes introducing edits in the HBG1/2 promoter which block binding of the BCL11A repressor protein. In the third strategy (lower), the gene encoding BCL11A (or an enhancer required for its transcription) is inactivated by gene editing. In the fourth strategy, editing is used to activate the GATA1 enhancer sequence which activates fetal hemoglobin production.

FIG. 6A illustrates the structure of adult hemoglobin—a quaternary-subunit protein comprising two subunits of hemoglobin subunit beta (β1 and β2) and two subunits of hemoglobin subunit alpha (α1 and α2) versus the structure of fetal hemoglobin—which comprises a hemoglobin subunit A-gamma (HBG1) and a subunit G-gamma (HBG2) in place of the two beta subunits. Fetal hemoglobin production is suppressed in normal adults due to the negative regulation of transcription of HBG1 and HBG2 genes which form the two gamma subunits by various molecular regulators, including BCL11A. In healthy humans, a shift from γ-globin to β-globin gene expression around birth underlies the switch from fetal (α2γ2; HbF) to adult (α2β2; HbA) hemoglobin. By 6 months of age the major hemoglobin is HbA.

FIG. 6B illustrates the hemoglobin gene locus on chromosome 11, including various hemoglobin genes (e.g., those encoding the beta, gamma, and alpha subunits), the location of the SCD mutation in the gene encoding the beta subunit, and the β-LCR transcription regulatory region.

FIG. 7 represents a first strategy that may be achieved using the LNP editing systems disclosed herein. In this strategy, a disclosed editing system is employed to introduce an edit that blocks or disrupts the binding site for BCL11A or LRF (or both) in the HBG1 and HBG2 promoters. BCL11A and LRF are transcriptional repressors that contribute to fetal-to-adult hemoglobin switching. Restoring HbF expression by inhibiting BCL11A (or LRF) function has been shown to correlate with positive outcomes for sickle cell disease patients. Strategy 1: disrupt the BCL11A binding site in the HBG1/2 promoters→reducing BCL11A repression of HBG1/2 expression→increased production of HBG1/2→upregulating HbF.

FIG. 8 represents a second strategy that may be achieved using the LNP editing systems disclosed herein. In this strategy, a disclosed editing system is employed to introduce an edit that blocks or disrupts the production of BCL11A (e.g., by inactivating the gene encoding BCL11A in the coding region or in a regulatory region), or by inactivating GATA-1, a transcriptional activator or BLCA11 transcription. BCL11A is a transcriptional repressor that contributes to fetal-to-adult hemoglobin switching. Restoring HbF expression by inhibiting BCL11A function has been shown to correlate with positive outcomes for sickle cell disease patients. Strategy 2: disrupt BCL11A coding region (“X”) or the erythroid-specific enhancer, GATA-1 (“star”) of BCL11A gene→reducing BCL11A (repressor of HbF)→upregulating HbF.

FIG. 9 represents a third strategy that may be achieved using the LNP editing systems (e.g., a base editor) disclosed herein. In this strategy, a disclosed editing system (e.g., a base editor) is employed to introduce an edit that converts A to G at −113 in the HBG1/2 promoters, which activates a GATA-1 binding site, which activates or enhances the expression of HBG1/2 subunits, and thus, HbF up-production. Strategy 3: base edit-catalyzed conversion of A to G at −113 in HBG1/2 promoters to activate a GATA-1 enhancer binding site, which enhances expression of HBG1/2 subunits, and thus, HbF up-production.

FIG. 10 represents a process for improving a programmable nuclease, such as, but not limited to a type V ortholog (e.g., ID405), by introducing one or more arginine residues in place of a naturally-occurring non-arginine residues at positions predicted to have an impact on the interaction between the ID405 amino acid sequence and a guide RNA (prediction based on computational methodology of Example 13). The substitutable positions are then ranked in accordance with the magnitude of stabilizing energetics associated with arginine replacement at any particular position as outline in Example 13. Variant type V editors are then engineered and tested to determine which variants provide for any improvement in editing efficiency. Results are shown in Example 13.

FIG. 11 compares the interaction between Lys292 in ID405-1 with a replaced Arg292 with the guide RNA showing a more favorable interaction with the Arg292 residue, as described in Example 13.

FIG. 12 compares the interaction between Gln492 in ID405-1 with a replaced Arg492 with the guide RNA showing a more favorable interaction with the Arg492 residue, as described in Example 13.

FIG. 13 compares the interaction between Asn770 in ID405-1 with a replaced Arg770 with the guide RNA showing a more favorable interaction with the Arg770 residue, as described in Example 13.

FIG. 14 provides a graph showing the editing efficiency in primary HSPCs of select ID405-1 variants with arginine substitution mutations. WT designated ID405-1. Each variant enzyme, e.g., S972R, designates ID405-1 with a particular arginine substitution (e.g., S972R designates that serine at position 972 relative to ID405-1 has been substituted with an arginine). See Example 13 for details.

FIG. 15 is a schematic of ID405-1 variants comprising one or more NLS and optional linkers to improve ID405-1 editing activity, in accordance with Example 14.

FIG. 16 is a schematic that shows that modified ID405-1 variant containing various linkers and NLSs in various configurations at the N- and/or C-termini improves editing as compared to baseline Construct 1 in primary HSPCs by up to a 5-fold increase. See Example 14 for details.

FIG. 17A is a schematic representation of the selection scheme for ID405 used in Example 15. The ID405 plasmid library was transformed into E. coli cells. The most active variants cut ccdB encoding plasmid which led to its subsequent degradation. This allowed for E. coli to grow on selective media promoting ccdB induction. Cells that receive plasmid with WT or non-functional/less active ID405 mutant died due to activity of ccdB.

FIG. 17B is a schematic of the ID405 containing plasmid pACYCDuet_ID405-HBG1-crRNA-TTR-HDV used for directed evolution.

FIG. 17C is a schematic of the domain structure of ID405. Domains are labeled in the protein schematic. Marked segments above the protein denote regions selected for mutagenesis.

FIG. 18 is a set of photos of test assays evaluating the performance of ID405 constructs against ccdB containing selection plasmids with TCTT and TTGG PAM sequences upstream of the HBG1 target.

FIG. 19A is a scatter plot summarizing variant frequency in N-terminus library under the selection against TCTT PAM. Nucleotide positions denote the amplified sequence used for library preparation.

FIG. 19B is a scatter plot summarizing variant frequency in N-terminus library under the selection against TTGG PAM. Nucleotide positions denote the amplified sequence used for library preparation.

FIG. 19C is a scatter plot summarizing variant frequency in N-terminus library under the selection against TTGG PAM under the presence of 0.1 mM IPTG. Nucleotide positions denote the amplified sequence used for library preparation.

FIG. 19D is a scatter plot summarizing variant frequency in C-terminus library under the selection against TCTT PAM. Nucleotide positions denote the amplified sequence used for library preparation.

FIG. 19E is a scatter plot summarizing variant frequency in C-terminus library under the selection against TTGG PAM. Nucleotide positions denote the amplified sequence used for library preparation.

FIG. 19F is a scatter plot summarizing variant frequency in C-terminus library under the selection against TTGG PAM under the presence of 0.1 mM IPTG. Nucleotide positions denote the amplified sequence used for library preparation.

FIG. 19G is a scatter plot summarizing variant frequency in N-2 library under the selection against TCTT PAM. Nucleotide positions denote the amplified sequence used for library preparation.

FIG. 19H is a scatter plot summarizing variant frequency in N-2 library under the selection against TTGG PAM. Nucleotide positions denote the amplified sequence used for library preparation.

FIG. 19I is a scatter plot summarizing variant frequency in N-2 library under the selection against TTGG PAM under the presence of 0.1 mM IPTG. Nucleotide positions denote the amplified sequence used for library preparation.

FIG. 19J is a scatter plot summarizing variant frequency in C-2 library under the selection against TCTT PAM. Nucleotide positions denote the amplified sequence used for library preparation.

FIG. 19K is a scatter plot summarizing variant frequency in C-2 library under the selection against TTGG PAM. Nucleotide positions denote the amplified sequence used for library preparation.

FIG. 19L is a scatter plot summarizing variant frequency in C-2 library under the selection against TTGG PAM under the presence of 0.1 mM IPTG. Nucleotide positions denote the amplified sequence used for library preparation.

FIG. 19M is a scatter plot summarizing variant frequency in a repeated selection of N-2 library under the selection against TTGG PAM using a greater number of E. coli transformants in round 1. Nucleotide positions denote the amplified sequence used for library preparation.

FIG. 19N is a scatter plot summarizing variant frequency in a repeated selection of N-2 library under the selection against TTGG PAM under the presence of 0.1 mM IPTG using a greater number of E. coli transformants in round 1. Nucleotide positions denote the amplified sequence used for library preparation.

FIG. 20A, FIG. 20B, and FIG. 20C show the computationally predicted optimal 2′-OMe guide modification sites in guide RNA for lbCas12a over the invariant region of nucleotides 1-20 (FIG. 20A), in guide RNA for asCas19 over the invariant region of nucleotides 1-19 (FIG. 20B), and in guide RNA for fnCas12 over the scaffold region of nucleotides 1-19 (FIG. 20C). Lower case letters (boxes with white background fill) denote sites selected by the algorithm that have 2′-OMe modifications. Upper case letters (boxes with grey background fill) denote nucleotides sites that are not modified. Scores next to each sequence (one per row) denote the potential loss of hydrogen bonding interactions were that position to be modified with a 2′-OMe modification. Scores over each nucleotide (one per column) denote the averaged hydrogen bond interaction calculated for that guide nucleotide position. Three exemplary modified guides with varying scores (high; med; low) are also given for each Type V family member (indicated by black stars). The top row guide sequence is modified at every position with 2′-OMe modifications.

FIG. 21A, and FIG. 21B show optimal 2-OMe guide modification sites in spacer region at nucleotides 20-39 of the asCas12 guide sequence (FIG. 21A) and at nucleotides 20-39 of the fnCas12 guide sequence (FIG. 21B). Lower case letters (boxes with white background fill) denote sites selected by the algorithm that may have 2′-OMe modifications. Upper case letters (boxes with grey background fill) denote nucleotides sites that are not modified. Scores next to each sequence (one per row) denote the potential loss of hydrogen bonding interactions due modifications. Scores over each nucleotide (one per column) denote the averaged hydrogen bond interaction calculated for that guide nucleotide. Three-four exemplary modified guides with varying scores are also given for each Type V family member (indicated by black stars).

FIG. 22A and FIG. 22B illustrate results of MOE analysis performed with Cas12a guide bound protein structure. Based on MOE structural protocol, nucleotide positions are identified where the 2′-OH of gRNA nucleotide is making contact with Cas12 protein. Dots indicate sum of all interactions at those positions between the 2′OH group of a given nucleotide and either the guide itself or with the protein and represent positions that ought not to be modified.

FIG. 23A and FIG. 23C illustrate results of MOE analysis performed with Cas12a guide bound protein structure. Based on MOE structural protocol, nucleotide positions are identified where the 2′-OH of gRNA nucleotide is making contact with Cas12 protein. Dots indicate sum of all interactions at those positions between the 2′OH group of a given nucleotide and either the guide itself or with the protein and represent positions that ought not to be modified.

FIG. 24 illustrates, based on the computation methodology of Example 16, nucleotide sites along the length of a type V guide RNA which may permissibly be modified with 2′-OMe modifications.

FIG. 25, seven different chemical modification patterns are designed for gRNA of three different targets (target 1=PCSK9 gene; target 2=B2M gene; target 3=BCL11A binding site in the HBG1/2 promoters. Targeting this site with a CRISPR-Cas editor and guide RNA can inactivate the binding site of the BCL11A transcriptional repressor, thereby increasing transcription of IIBG1/2 genes, which results in increased production of HBG1/2 subunits, and consequently, increased production of HbF. Each of these three guides have same direct repeat sequence (UGAAUUUCUACUGUUGUAGAU) but have different spacer length for unique DNA targets. In every guide, first two and last two phosphates have been converted to phosphorothioate. According to MOE, position 3 may not be suitable for phosphorothioate modification (FIGS. 26 and 27) and hence kept unmodified. For this work, it was decided to work with 405-1 gRNA which has UG dinucleotide added before Cas12 guide sequence. For this work, these two nucleotides are assigned with −2 and −1 number. Following nucleotide sequence is identical with Cas12a gRNA direct repeat (AAUUUCUA . . . ) and they are assigned with 1, 2, 3 . . . as nucleotide identification number.

FIG. 26 shows the permissible sites to be modified based on detecting the strength of phosphate interactions for FnCas12a/guide complexes.

FIG. 27 shows the permissible sites to be modified based on detecting the strength of phosphate interactions for Cas12a/guide complexes.

FIG. 28 shows a summary of modified guides tested in vitro in Example 17. The modified guides Mod1, Mod4, Mod5, Mod6, and Mod7 were tested in accordance with the methodology depicted in FIG. 29.

FIG. 29 shows Methodology for testing modified guide RNAs as detailed in Example 17.

FIG. 30 shows results showing high editing rates in primary HSCs at a clinically relevant locus (up to 77% editing at hB2M locus) as outlined in Example 17.

FIG. 31 is a schematic showing an RNP complex (based on Cas9) wherein the nuclease component comprises an NLS (PKKKRKV). The complex forms in the nuclease and relocates to the nuclease as facilitated by the NLS.

FIG. 32 depicts the concept of a PNA-NLS probe to couple an NLS directly to a guide RNA. Here, the exemplary PNA is 9 residues in length which is joined through an optional linker to one or more NLS.

FIG. 33 depicts the concept of a PNA-NLS probe to couple an NLS directly to a guide RNA at an additional sequence element added to the guide RNA referred to as the PNA binder element. This may be configured at either end of the guide RNA. Here, the exemplary PNA is 9 residues in length which is joined through an optional linker to one or more NLS.

FIG. 34A shows an exemplary guide RNA targeting TTR gene and having the sequence from 5′ to 3′ of (SEQ ID NO: 751), wherein the bolded sequence hybridizes to the PNA having the nucleic acid sequence AGCCACGAAAA (SEQ ID NO: 753) fused to the NLS peptide PKKKRKV.

FIG. 34B shows an exemplary guide RNA targeting TTR gene and having the sequence from 5′ to 3′ of (SEQ ID NO: 751), wherein the bolded sequence hybridizes to the PNA having the nucleic acid sequence CCACGAAAA fused to the NLS peptide PKKKRKV.

FIG. 34C shows an exemplary guide RNA targeting any gene and having the sequence from 5′ to 3′ of (SEQ ID NO: 752), wherein the bolded sequence hybridizes to the PNA having the nucleic acid sequence AGCCACGAAAA (SEQ ID NO: 753) fused to the NLS peptide PKKKRKV, where in “N” designates any nucleotide and will depend upon the target sequence being targeted by the guide RNA.

FIG. 35 is a graph of % indel formation by 405-1 nuclease with various modified gRNAs in a human HSPC donor. These results show that RNA extensions and DNA extensions can improve editing at the HBG locus 3-10 fold in an all RNA format relative to the mod6 benchmark using the 405-1 nuclease.

FIG. 36 is a graph of % indel formation by 405-1 nuclease with various modified gRNAs in a human HSPC donor. These results show that RNA extensions and DNA extensions do not improve editing at the B2M locus in an all RNA format relative to the mod6 benchmark using the 405-1 nuclease.

FIG. 37 shows a graph of % indel formation by 405-1 nuclease with various modified gRNAs in a human HSPC donor at the HBG locus. These results show that RNA and DNA extensions are not compatible with the Type V mod6 schema but guides modified with mod7 schema can tolerate RNA extensions.

FIG. 38 is modification 6 schema. This figure outlines the positions of 2′-OMe modifications and phosphothiorate modifications in gRNAs with the modification 6 schema. Light grey and uppercase letters=Unmodifed; Dark grey and lowercase letters=modified with phosphothiorate (or in the alternative, indicated below with a post-nt asterisk); White and lowercase letters=modified with 2′-Ome. The sequences of FIG. 38 are:
gRNA0260: u*g*AAUuuCUacUguuGuagaUcCUUGUCaagGcUAUUGGU*c*a (SEQ ID NO: 680) (aka “mod6” guide), wherein A, G, U, C=unmodified RNA nucleotide; a, g, u, c=2′-O-Methyl-nucleotide; *=Phosphorothioate linkage
gRNA0390: u*g*AAUuuCUacUguuGuaGAucCUUGUCaaggcuAUUGGu*c*a (SEQ ID NO: 681), wherein A, G, U, C=unmodified RNA nucleotide; a, g, u, c=2′-O-Methyl-nucleotide; *=Phosphorothioate linkage
gRNA0391: u*g*AAUUuCUacUguuGuaGAUcCUUGUCaaggCUAUUGGu*c*a (SEQ ID NO: 682), wherein A, G, U, C=unmodified RNA nucleotide; a, g, u, c=2′-O-Methyl-nucleotide; *=Phosphorothioate linkage
gRNA0392: u*g*AAUUuCUacUguuGUAGAUcCUUGUCaagGCUAUUGGu*c*a (SEQ ID NO: 683), wherein A, G, U, C=unmodified RNA nucleotide; a, g, u, c=2′-O-Methyl-nucleotide; *=Phosphorothioate linkage

FIG. 39 shows a graph of % indel formation by 405-1 nuclease with various modified gRNAs in a human HSPC donor at the HBG locus. These results show that decreasing the proportion of modification in a gRNA can increase editing 4-5 fold in vitro in CD34+ human HSPCs.

FIG. 40 shows a graph of % indel formation at the HBG locus in human CD34+ HSPCs by gRNAs with 3′ extensions that create a hairpin with the spacer region of the gRNA. The results show 3′ extensions creating hairpins with a type V spacer (backfolds) increased editing above the mod6 benchmark gRNA at the HBG locus in human HSPCs. gRNA0260 is “mod6” having the sequence and modification scheme of u*g*AAUuuCUacUguuGuagaUcCUUGUCaagGcUAUUGGU*c*a (SEQ ID NO: 680) (aka “mod6” guide), wherein A, G, U, C=unmodified RNA nucleotide; a, g, u, c=2′-O-Methyl-nucleotide; *=Phosphorothioate linkage.

FIG. 41 shows a graph of % indel formation by 405-1 with modified gRNAs across three different human HSPC donors at the HBG locus. The results showed that the effect of modified gRNAs on indel formation is consistent across HSPC donors using 405-1 3x NLS nuclease. For each cluster of graphs, the samples appear in the same ordering of (a) Donor 310, (b) Donor 309, and (c) Donor 308.

FIG. 42 shows a graph of % indel formation at the HBG locus by modified gRNAs across 405-1 protein variants in human hematopoietic stem cells. The results show that the addition of the point mutant K292R and a 3×NLS increased indel formation across all modified gRNAs tested. gRNAs containing a 5′ DNA extension of a 15 bp randomized sequence improved indel formation by up to ~8 fold. Other modified gRNAs tested show more modest increases in indel formation. For each cluster of graphs, the samples appear in the same ordering of (a) 405-1, (b) 405-1 3x NLS, (c) 4051-E1014R+3×NLS, and (d) 405-1 K292R+3×NLS.

FIG. 43 comprises two panels: (A) A schematic representation of luciferase mRNA with miRNA target sites (ts) inserted in the 3′ UTR. Luciferase mRNA consists of 5′ cap, 5′UTR, luciferase coding sequence, 3′ UTR followed by a poly A tail. A single copy (×1) or three copies (×3) of miRNA ts were inserted at the 3′ end of the 3′ UTR. An alternative (alt) insertion site is located at the 5′ end of the 3′ UTR, near the luciferase coding sequence. When combining two miRNA target sites in the same UTR, the first miRNA target site is inserted at the ‘alt’ location, and the second miRNA target site is inserted at the 3′ end of the UTR. (B) Incorporation of miR-122 ts into the 3′ UTR of luciferase mRNA results in the suppression of encoded protein in the Huh7 hepatocyte cell line, where miR-122 is expressed. A single copy of the target site at the end of 3′ UTR near the poly A tail is sufficient to suppress luciferase expression by 5-fold compared to the control. Increasing the number of copies to three does not further enhance suppression. However, when the target site is inserted at the 5′ end of the UTR, near the coding sequence, suppression is enhanced to 10-fold. In contrast, insertion of the target site of miR-142, which is not expressed in hepatocyte cell line, has no impact on luciferase expression by itself and does not influence miR-122 mediated suppression when both target sites are inserted together on the same UTR.

FIG. 44 comprises two panels: (A) A schematic structure of luciferase mRNA with miRNA target sites (ts) inserted in the 3′ UTR. Luciferase mRNA consists of 5′ cap, 5′UTR, luciferase coding sequence, 3′ UTR followed by poly A tail. In this structure, three copies of miRNA target site are inserted at the 5′ end of 3′ UTR, near luciferase coding sequence. When combining two miRNA target sites in the same UTR, the first miRNA target site is inserted near the luciferase coding sequence, and the second miRNA target site is inserted at the 3′ end of UTR. (B) Cell type-specific suppression of luciferase expression was achieved by incorporating appropriate miRNA ts in the 3′ UTR. miR-122 ts inserted in the 3′ UTR of luciferase mRNA led to the suppression of protein expression in a hepatocyte cell line, where miR-122 is expressed (left), while no suppression was observed in a monocyte cell line, where miR-122 is not expressed (right). Similarly, miR-142 ts suppressed luciferase expression exclusively in the monocyte cell line, where miR-142 is expressed. Insertion of miR-122 and miR-142 target sites in the same UTR inhibits luciferase expression in both hepatocyte and monocyte cell line.

FIG. 45 shows the miRNAs in Table 1 of Example 22 were evaluated for their target-site mediated suppression in various immune cell lines and CD34+CD38-CD90+CD45RA-Lin-long-term hematopoietic stem cells. While the insertion of liver specific miR-122 or epithelial specific miR-200b, 200c, 203a and 205 target sites in the 3′ UTR of cargo does not affect its expression in immune cells, hematopoietic miRNAs suppress cargo expression in one or more cell types. miR-142 and let7e effectively suppress cargo expression in all cell lines. miR-223 exhibits mild activity in all cell lines, with the least effect observed in long-term hematopoietic cells. miR-155 demonstrates suppression in the monocyte cell line and in long-term hematopoietic cells, albeit weaker in the latter. miR-342 suppresses expression in both monocyte and T-cell lines. Both miR-126 5p and 3p, which are abundantly expressed in endothelial cells, show a substantial suppressive effect in long-term hematopoietic stem cells.

### DETAILED DESCRIPTION

### I. Introduction

Described herein are LNP compositions comprising gene editing systems for use in treating disease (e.g., a hemoglobinopathy) and/or otherwise modifying the sequence and/or expression of target nucleotide sequences, such as a hemoglobin gene or regulatory region thereof. The disclosure provides LNPs capable of delivering a gene editing system to red blood cell precursor cells, including but not limited to hematopoietic stem cells (HSCs). The gene editing systems of the present disclosure are preferably delivered to a patient under in vivo conditions (e.g., administered to a subject in an effective amount).

The disclosure also provides in various aspects therapeutic or pharmaceutical compositions comprising LNPs comprising gene editing systems or one or more components thereof for use in treating disease (e.g., a beta-hemoglobinopathy) and/or otherwise modifying the sequence and/or expression of target nucleotide sequences. The gene editing systems may comprise DNA components, RNA components, protein components, nucleoprotein components, or combinations thereof. In other aspects, the disclosure provides nucleic acid molecules that encode various componentry of the deliverable gene editing systems contemplated herein. In addition, other aspects of the disclosure provide nucleic acid molecules as components of the herein contemplated gene editing systems, such as, but not limited to plasmids or vectors encoding one or more components of a gene editing system, RNAs encoding one or more components of a gene editing system (e.g., mRNAs coding for a nuclease domain of a gene editing system), and non-coding RNAs (e.g., guide RNAs capable of complexing with and targeting a nucleic acid-programmable DNA binding domain to a specific target nucleotide sequence or a retron ncRNA). The disclosure in other aspects provides for the various protein components of the various gene editing systems contemplated herein, including, but not limited to, user-programmable DNA binding proteins and various effector proteins, such as nucleases, polymerases, reverse transcriptases, recombinases, integrases, endonucleases, exonucleases, transposases, and deaminases. The disclosure also describes nucleoprotein components of the gene editing systems contemplated herein, such as, but not limited to a nuclease-guide RNA complexes. The disclosure also provides methods of modifying the sequence and/or expression level of a target nucleic acid molecule through the delivery and/or administration of an LNP described herein that comprises a gene editing system or components thereof. Still further, the disclosure provides methods of treating a disease by administering a therapeutically effective amount of an LNP-based gene editing system that results in the modification in the sequence and/or expression level of a target nucleic acid molecule (e.g., a disease-associated gene).

The LNP compositions and/or gene editing systems described herein may include a variety of coding RNA molecules that code for the various components of gene editors. In various aspects, the coding RNA may be linear mRNA. In other embodiments, the coding RNA may be circular mRNA. In various aspects, the improved LNPs protect linear and/or circular mRNA cargos from degradation and clearance while achieving targeted systemic or local delivery for use as enhanced gene editing platforms and/or therapeutic agents.

In various other aspects, the LNP compositions and/or gene editing systems described herein may also include a repair template, e.g., an HDR donor single or double stranded DNA.

Accordingly, the instant specification describes compositions, methods, processes, kits and devices for the selection, design, preparation, manufacture, formulation, and/or use of LNP-based gene editing systems as therapeutic compositions for the treatment of hemoglobinopathies. Further described herein are compositions, methods, processes, kits and devices for the selection, design, preparation, manufacture, formulation, and/or use of LNP-based gene editing therapeutics for the prophylactic and/or therapeutic treatment of one or more diseases or a symptom thereof. The components capable of being encapsulated by or otherwise incorporated by the LNPs described herein may be referred to as LNP “payloads” and may include all of the biological materials described above, including DNA molecules, RNA molecules (coding and/or non-coding), proteins, and nucleoproteins (e.g., Cas/guide RNA complexes).

In certain embodiments, the LNP compositions selectively and effectively deliver the gene editing payloads to specific cell types that allow for the hemoglobinopathies to be treated. In certain embodiments, the LNPs of the present disclosure deliver to red blood cell progenitor cells. In certain embodiments, the LNPs of the present disclosure deliver to hematopoietic stem cells.

### II. LNP Delivery Systems

The payloads (e.g., linear and circular mRNAs; nucleobase editing systems and/or components thereof) described herein may be encapsulated and delivered by lipid nanoparticles (LNPs) and compositions and/or formulations comprising RNA-encapsulated LNPs.

Below describes LNPs that may be used as the payload delivery vehicles contemplated herein, as well as the various ionizable lipids, structural lipids, PEGylated lipids, and phospholipids that may be used to make the herein LNPs for delivery payloads to cells. In addition, below describes additional LNP components that are contemplated, such as targeting moieties and other lipid components.

### A. Lipid Nanoparticle Compositions

In one aspect, the present disclosure further provides delivery systems for delivery of a therapeutic payload (e.g., the RNA payloads described herein which may encode a polypeptide of interest, e.g., a nucleobase editing system or a therapeutic protein) disclosed herein. In some embodiments, a delivery system suitable for delivery of the therapeutic payload disclosed herein comprises a lipid nanoparticle (LNP) formulation.

In some embodiments, an LNP of the present disclosure comprises an ionizable lipid, a structural lipid, a PEGylated lipid (aka PEG lipid), and a phospholipid. In alternative embodiments, an LNP comprises an ionizable lipid, a structural lipid, a PEGylated lipid (aka PEG lipid), and a zwitterionic amino acid lipid. In some embodiments, an LNP further comprises a 5th lipid, besides any of the aforementioned lipid components. In some embodiments, the LNP encapsulates one or more elements of the active agent of the present disclosure. In some embodiments, an LNP further comprises a targeting moiety covalently or non-covalently bound to the outer surface of the LNP. In some embodiments, the targeting moiety is a targeting moiety that binds to, or otherwise facilitates uptake by, cells of a particular organ system.

In some embodiments, an LNP has a diameter of at least about 20 nm, 30 nm, 40 nm, 50 nm, 60 nm, 70 nm, 80 nm, or 90 nm. In some embodiments, an LNP has a diameter of less than about 100 nm, 110 nm, 120 nm, 130 nm, 140 nm, 150 nm, or 160 nm. In some embodiments, an LNP has a diameter of less than about 100 nm. In some embodiments, an LNP has a diameter of less than about 90 nm. In some embodiments, an LNP has a diameter of less than about 80 nm. In some embodiments, an LNP has a diameter of about 60-100 nm. In some embodiments, an LNP has a diameter of about 75-80 nm.

In some embodiments, the lipid nanoparticle compositions of the present disclosure are described according to the respective molar ratios of the component lipids in the formulation. In some embodiments, the mol-% of the ionizable lipid is from about 10 mol-% to about 80 mol-%. In some embodiments, the mol-% of the ionizable lipid is from about 20 mol-% to about 70 mol-%. In some embodiments, the mol-% of the ionizable lipid is from about 30 mol-% to about 60 mol-%. In some embodiments, the mol-% of the ionizable lipid is from about 35 mol-% to about 55 mol-%. In some embodiments, the mol-% of the ionizable lipid is from about 40 mol-% to about 50 mol-%.

In some embodiments, the mol-% of the phospholipid is from about 1 mol-% to about 50 mol-%. In some embodiments, the mol-% of the phospholipid is from about 2 mol-% to about 45 mol-%. In some embodiments, the mol-% of the phospholipid is from about 3 mol-% to about 40 mol-%. In some embodiments, the mol-% of the phospholipid is from about 4 mol-% to about 35 mol-%. In some embodiments, the mol-% of the phospholipid is from about 5 mol-% to about 30 mol-%. In some embodiments, the mol-% of the phospholipid is from about 10 mol-% to about 20 mol-%. In some embodiments, the mol-% of the phospholipid is from about 5 mol-% to about 20 mol-%. In some embodiments, the mol-% of the phospholipid is from about 20 mol-% to about 60 mol-%. In some embodiments, the mol-% of the phospholipid is from about 30 mol-% to about 50 mol-%. In some embodiments, the mol-% of the phospholipid is from about 35 mol-% to about 45 mol-%. In some embodiments, the LNP comprises a mixture of two or more phospholipids that cumulatively account for any of the aforementioned mol-%.

In some embodiments, the mol-% of the structural lipid is from about 10 mol-% to about 80 mol-%. In some embodiments, the mol-% of the structural lipid is from about 20 mol-% to about 70 mol-%. In some embodiments, the mol-% of the structural lipid is from about 30 mol-% to about 60 mol-%. In some embodiments, the mol-% of the structural lipid is from about 35 mol-% to about 55 mol-%. In some embodiments, the mol-% of the structural lipid is from about 40 mol-% to about 50 mol-%.

In some embodiments, the mol-% of the PEG lipid is from about 0.1 mol-% to about 10 mol-%. In some embodiments, the mol-% of the PEG lipid is from about 0.2 mol-% to about 5 mol-%. In some embodiments, the mol-% of the PEG lipid is from about 0.5 mol-% to about 3 mol-%. In some embodiments, the mol-% of the PEG lipid is from about 1 mol-% to about 2 mol-%. In some embodiments, the mol-% of the PEG lipid is from about 2 mol-% to about 5 mol-%. In some embodiments, the mol-% of the PEG lipid is from about 2 mol-% to about 3 mol-%. In some embodiments, the mol-% of the PEG lipid is about 1.5 mol-%. In some embodiments, the mol-% of the PEG lipid is about 2 mol-%. In some embodiments, the mol-% of the PEG lipid is about 2.5 mol-%. In some embodiments, the mol-% of the PEG lipid is about 3 mol-%. In some embodiments, the mol-% of the PEG lipid is about 3.5 mol-%.

i. Ionizable Lipids

In some embodiments, an LNP disclosed herein comprises an ionizable lipid. In some embodiments, an LNP comprises two or more ionizable lipids.

Described below are a number of exemplary ionizable lipids of the present disclosure.

In some embodiments, an LNP of the present disclosure comprises an ionizable lipid disclosed in one of US 2023/0053437; US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, an LNP described herein comprises a lipid, e.g., an ionizable lipid, disclosed in US Application publication US2017/0119904, which is incorporated by reference herein, in its entirety.

In some embodiments, an LNP described herein comprises a lipid, e.g., an ionizable lipid, disclosed in PCT Application publication WO2021/204179, which is incorporated by reference herein, in its entirety.

In some embodiments, an LNP described herein comprises a lipid, e.g., an ionizable lipid, disclosed in PCT Application WO2022/251665A1, which is incorporated by reference herein, in its entirety.

In some embodiments, an LNP described herein comprises an ionizable lipid of Table Z:

TABLE Z
Exemplary Ionizable Lipids

| Com- | |
|---|---|
| pound # | Structure |
| L-1 | 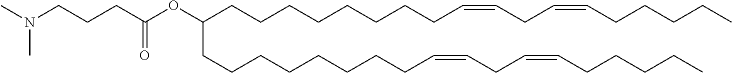 [SMILES: CCCCC/C=C\C/C=C\CCCCCCCCC(CCCCCCCC/C=C\C/C=C\CCCCC)OC(=O)CCCN(C)C] |
| L-2 | 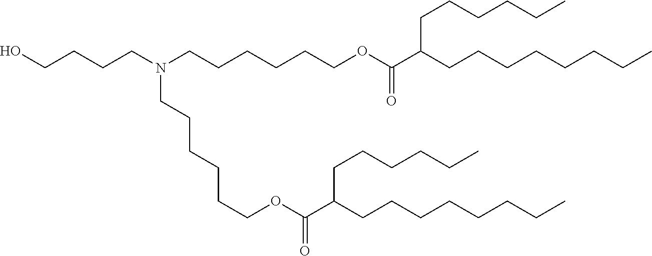 [SMILES: CCCCCCCCC(CCCCCC)C(=O)OCCCCCCN(CCCCO)CCCCCCOC(=O)C(CCCCCC)CCCCCCCC] |
| L-3 | 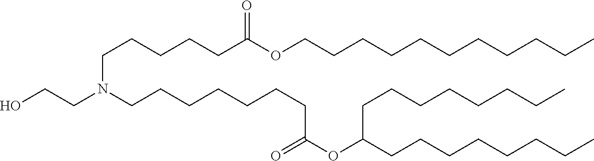 [SMILES: CCCCCCCCCCCOC(=O)CCCCCN(CCO)CCCCCCCC(=O)OC(CCCCCCCC)CCCCCCCC] |
| L-4 | 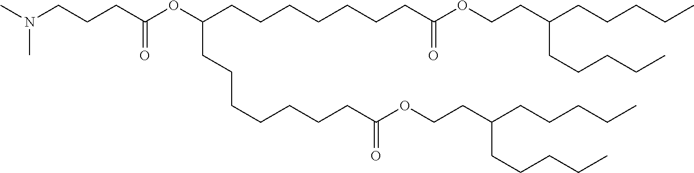 [SMILES: CCCCCC(CCCCC)CCOC(=O)CCCCCCCC(CCCCCCCC(=O)OCCC(CCCCC)CCCCC)OC(=O)CCCN(C)C] |
| L-5 | 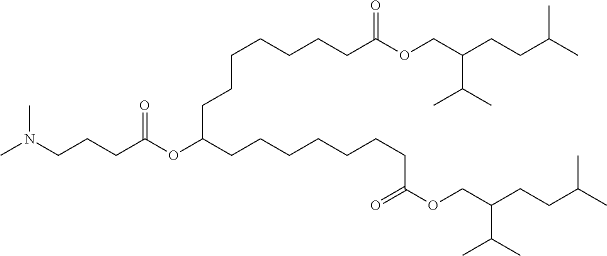 [SMILES: CC(C)CCC(COC(=O)CCCCCCCC(CCCCCCCC(=O)OCC(CCC(C)C)C(C)C)OC(=O)CCCN(C)C)C(C)C] |
| L-6 | 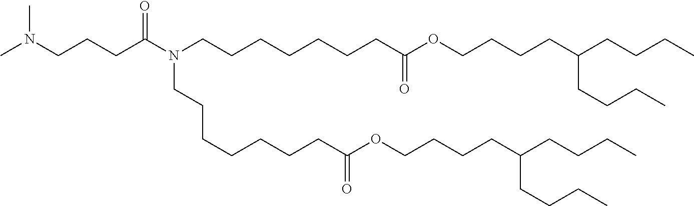 [SMILES: CCCCC(CCCC)CCCCOC(=O)CCCCCCCN(CCCCCCCC(=O)OCCCCC(CCCC)CCCC)C(=O)CCCN(C)C] |
| L-7 | 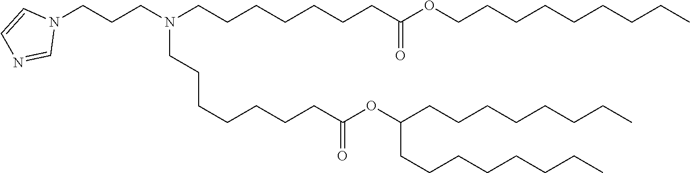 [SMILES: CCCCCCCCCOC(=O)CCCCCCCN(CCCCCCCC(=O)OC(CCCCCCCC)CCCCCCCC)CCCn1ccnc1] |
| L-8 | 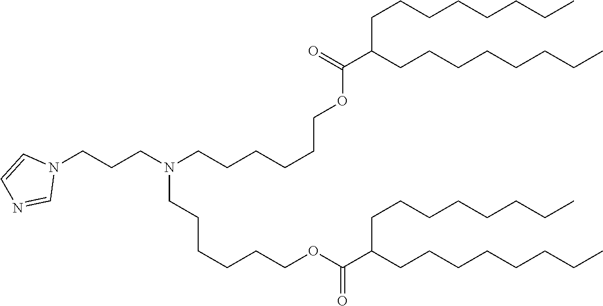 [SMILES: CCCCCCCCC(CCCCCCCC)C(=O)OCCCCCCN(CCCCCCOC(=O)C(CCCCCCCC)CCCCCCCC)CCCn1ccnc1] |
| L-9 | 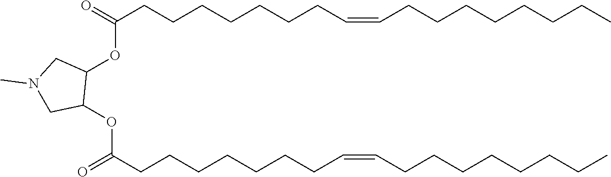 [SMILES: CCCCCCCC/C=C\CCCCCCCC(=O)OC1CN(C)CC1OC(=O)CCCCCCC/C=C\CCCCCCCC] |
| L-10 | 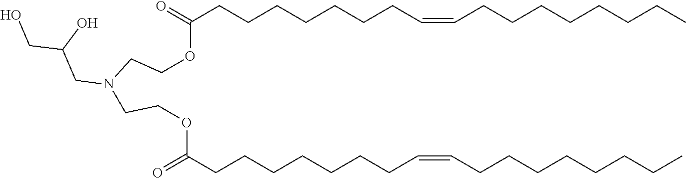 [SMILES: CCCCCCCC/C=C\CCCCCCCC(=O)OCCN(CCOC(=O)CCCCCCC/C=C\CCCCCCCC)CC(O)CO] |

In some embodiments, the ionizable lipid is MC3.

### Series “A”

In some embodiments, an LNP of the present disclosure comprises an ionizable lipid disclosed in PCT Application Publication WO2023044343A1, which is incorporated by reference herein, in its entirety.

### Formula (VII-A)

In some embodiments, ionizable lipids of the present disclosure have a structure of Formula (VII-A):

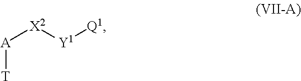

or a pharmaceutically acceptable salt thereof, wherein:
   A is —N(—X¹R¹)—, —C(R′)(-L¹-N(R″)R⁶)—, —C(R′)(—OR^{7a})—, —C(R′)(—N(R″)R^{8a})—, —C(R′)(—C(═O)OR^{9a})—, —C(R′)(—C(═O)N(R″)R^{10a})—, or —C(═N—R^{11a})—;
   T is —X^{2a}—Y^{1a}-Q^{1a} or —X³—C(═O)OR⁴;
   X¹ is optionally substituted C₂-C₆ alkylenyl;
   R¹ is —OH, —R^{1a},

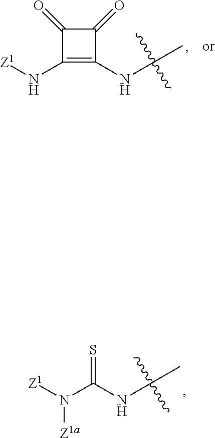

Z¹ is optionally substituted C₁-C₆ alkyl;
Z^{1a} is hydrogen or optionally substituted C₁-C₆ alkyl;
X² and X^{2a} are independently optionally substituted C₂-C₁₄ alkylenyl or optionally substituted C₂-C₁₄ alkenylenyl;
X³ is optionally substituted C₂-C₁₄ alkylenyl or optionally substituted C₂-C₁₄ alkenylenyl;
(i) Y¹ is

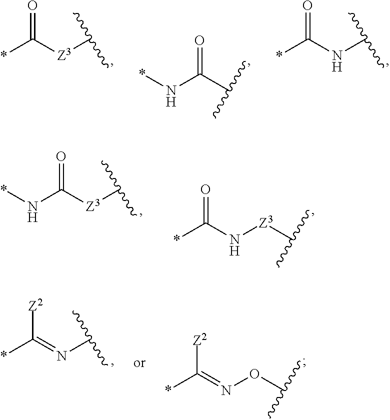

wherein the bond marked with an “*” is attached to X²;
   Y^{1a} is

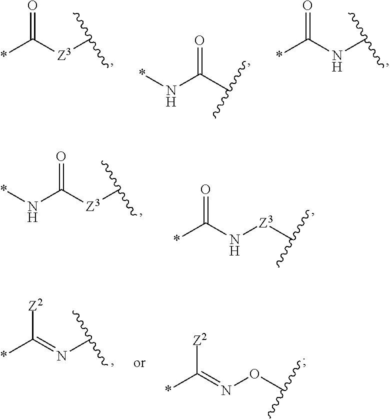

wherein the bond marked with an “*” is attached to X^{2a};
   each Z² is independently H or optionally substituted C₁-C₈ alkyl;
   each Z³ is independently optionally substituted C₁-C₆ alkylenyl;
   Q¹ is —NR²R³, —CH(OR²)(OR³), —CR²═C(R³)(R¹²), or —C(R²)(R³)(R¹²);
   Q^{1a} is —NR²R³, —CH(OR^{2′})(OR^{3′}), —CR²═C(R³)(R¹²), or —C(R^{2′})(R^{3′})(R^{12′}); or
   (ii) Y¹ is

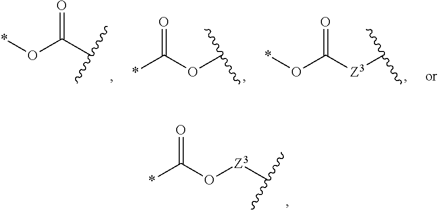

wherein the bond marked with an “*” is attached to X²;
   Y^{1a} is

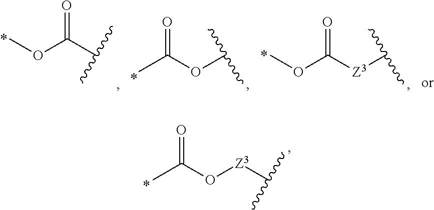

wherein the bond marked with an “*” is attached to X^{2a};
   each Z² is independently H or optionally substituted C₁-C₈ alkyl;
   each Z³ is independently optionally substituted C₁-C₆ alkylenyl;
   Q¹ is —NR²R³;
   Q^{1a} is —NR²′R³;
   R², R³, and R¹² are independently hydrogen, optionally substituted C₁-C₁₄ alkyl, optionally substituted C₂-C₁₄ alkenylenyl, or —(CH₂)ₘ-G-(CH₂)ₙH;
   R², R³, and R^{12′} are independently hydrogen, optionally substituted C₁-C₁₄ alkyl, optionally substituted C₂-C₁₄ alkenylenyl, or —(CH₂)ₘ-G-(CH₂)ₙH;
   G is a C₃-C₈ cycloalkylenyl;
   each m is independently 0, 1, 2, 3, 4, 5, 6, 7, 8, 9, 10, 11, or 12;
   each n is independently 0, 1, 2, 3, 4, 5, 6, 7, 8, 9, 10, 11, or 12;
   X³ is optionally substituted C₂-C₁₄ alkylenyl;
   R⁴ is optionally substituted C₄-C₁₄ alkyl;
   L¹ is C₁-C₈ alkylenyl;
   R⁶ is C₁-C₆ alkyl, (hydroxy)C₁-C₆ alkyl, or (amino)C₁-C₆ alkyl
   R^{7a} is —C(═O) N(R′″)R^{7b}, —C(═S)N(R′″)R^{7b}, —N═C(R^{7b})(R^{7c}), or

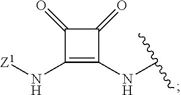

R^{7b} is C₁-C₆ alkyl, (hydroxy)C₁-C₆ alkyl, or (amino)C₁-C₆ alkyl;
R^{7c} is hydrogen or C₁-C₆ alkyl;
R^{8a} is —C(═O) N(R′″)R^{8b}, —C(═S)N(R′″)R^{8b}, —N═C(R^{8b})(R^{8c}), or

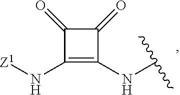

R^{8b} is C₁-C₆ alkyl, (hydroxy)C₁-C₆ alkyl, or (amino)C₁-C₆ alkyl;
R^{8c} is hydrogen or C₁-C₆ alkyl;
R^{9a} is —N═C(R^{9b})(R^{9c});
R^{9b} is C₁-C₆ alkyl, (hydroxy)C₁-C₆ alkyl, or (amino)C₁-C₆ alkyl;
R^{9c} is hydrogen or C₁-C₆ alkyl;
R^{10a} is —N═C(R^{10b})(R^{10c});
R^{10b} is C₁-C₆ alkyl, (hydroxy)C₁-C₆ alkyl, or (amino)C₁-C₆ alkyl;
R^{10c} is hydrogen or C₁-C₆ alkyl;
R^{11a} is —OR^{11b}, —N(R″)R^{11b}, —OC(═O)R^{11b}, or —N(R″)C(═O)R^{11b};
R^{11b} is C₁-C₆ alkyl, (hydroxy)C₁-C₆ alkyl, or (amino)C₁-C₆ alkyl;
R′ is hydrogen or C₁-C₆ alkyl;
R″ is hydrogen or C₁-C₆ alkyl; and
R′″ is hydrogen or C₁-C₆ alkyl.

### Formula (VIII-A)

In some embodiments, ionizable lipids of the present disclosure have a structure of Formula (VII-A), wherein the ionizable lipids of the present disclosure have a structure of Formula (VIII-A):

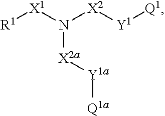

or a pharmaceutically acceptable salt thereof.

### Formula (X)

In some embodiments, ionizable lipids of the present disclosure have a structure of Formula (X):

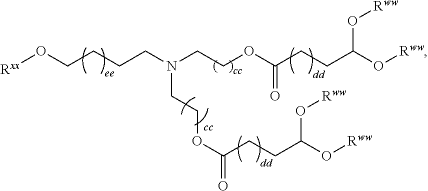

or a pharmaceutically acceptable salt thereof, wherein
each cc is independently selected from 3 to 9;
   R^{xx} is selected from hydrogen and optionally substituted C₁-C₆ alkyl; and
   (i) ee is 1,
      each dd is independently selected from 1 to 4; and
   each R^{ww} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any-(CH₂)₂— of the C₄-C₁₄ alkyl can be optionally replaced with C₂-C₆ cycloalkylenyl;
   (ii) ee is 0,
      each dd is 1; and
      each R^{ww} is linear C₄-C₁₂ alkyl.

In some embodiments, ionizable lipids of the present disclosure have a structure of Formula (X), wherein R^{xx} is H. In some embodiments, ionizable lipids of the present disclosure have a structure of Formula (X), wherein R^{X} is optionally substituted C₁-C₆ alkyl. In some embodiments, ionizable lipids of the present disclosure have a structure of Formula (X), wherein R^{xx} is C₁ alkyl. In some embodiments, ionizable lipids of the present disclosure have a structure of Formula (X), wherein R^{xx} is C₂ alkyl. In some embodiments, ionizable lipids of the present disclosure have a structure of Formula (X), wherein R^{xx} is C₃ alkyl. In some embodiments, ionizable lipids of the present disclosure have a structure of Formula (X), wherein R^{xx} is C₄ alkyl. In some embodiments, ionizable lipids of the present disclosure have a structure of Formula (X), wherein R^{xx} is C₅ alkyl. In some embodiments, ionizable lipids of the present disclosure have a structure of Formula (X), wherein R^{xx} is C₆ alkyl.

In some embodiments, ionizable lipids of the present disclosure have a structure of Formula (X), wherein each R^{ww} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any (CH₂)₂— of the C₄-C₁₄ alkyl can be optionally replaced with C₂-C₆ cycloalkylenyl. In some embodiments, ionizable lipids of the present disclosure have a structure of Formula (X), wherein each R^{ww} is C₄-C₁₄ alkyl, wherein any —(CH₂)₂— of the C₄-C₁₄ alkyl can be optionally replaced with C₂-C₆ cycloalkylenyl. In some embodiments, ionizable lipids of the present disclosure have a structure of Formula (X), wherein each R^{ww} is C₄-C₁₄ alkyl, wherein any —(CH₂)₂— of the C₄-C₁₄ alkyl can be optionally replaced with cyclopropylene. In some embodiments, ionizable lipids of the present disclosure have a structure of Formula (X), wherein each R^{ww} is branched C₄-C₁₂ alkenyl. In some embodiments, ionizable lipids of the present disclosure have a structure of Formula (X), wherein each R^{ww} is C₄-C₁₂ alkenyl comprising at least two double bonds. In some embodiments, ionizable lipids of the present disclosure have a structure of Formula (X), wherein each R^{ww} is C₉-C₁₂ alkenyl. In some embodiments, ionizable lipids of the present disclosure have a structure of Formula (X), wherein each R^{ww} is linear C₄-C₁₂ alkyl. In some embodiments, ionizable lipids of the present disclosure have a structure of Formula (X), wherein each R^{ww} is independently selected from the group consisting of C₆-C₁₄ alkyl, branched C₈-C₁₂ alkenyl, C₈-C₁₂ alkenyl comprising at least two double bonds, and C₉-C₁₂ alkenyl, wherein any —(CH₂)₂— of the C₆-C₁₄ alkyl can be optionally replaced with cyclopropylene. In some embodiments, ionizable lipids of the present disclosure have a structure of Formula (X), wherein each R^{ww} is C₆-C₁₄ alkyl, wherein any —(CH₂)₂— of the C₆-C₁₄ alkyl can be optionally replaced with cyclopropylene. In some embodiments, ionizable lipids of the present disclosure have a structure of Formula (X), wherein each R^{ww} is branched C₈-C₁₂ alkenyl, e.g., (linear or branched C₃-C₅ alkylenyl)-(branched C₅-C₇alkenyl), e.g., (branched C₅ alkylenyl)-(branched C₅alkenyl), e.g.,

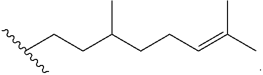 [SMILES: *CCC(C)CCC=C(C)C]

In some embodiments, ionizable lipids of the present disclosure have a structure of Formula (X), wherein each R^{ww} is C₈-C₁₂ alkenyl comprising at least two double bonds. In some embodiments, ionizable lipids of the present disclosure have a structure of Formula (X), wherein each R^{ww} is C₉-C₁₂ alkenyl.

In some embodiments, ionizable lipids of the present disclosure have a structure of Formula (X), wherein each R^{ww} is independently selected from the group consisting of C₆-C₁₄ alkyl (e.g., C₆, C₈, C₉, C₁₀, C₁₁, C₁₃ alkyl), wherein any —(CH₂)₂— of the C₆-C₁₄ alkyl can be optionally replaced with cyclopropylene.

In some embodiments, ionizable lipids of the present disclosure have a structure of Formula (X), wherein each R^{ww} is independently branched C₈-C₁₂ alkenyl (e.g., branched C₁₀ alkenyl).

In some embodiments, ionizable lipids of the present disclosure have a structure of Formula (X), wherein each R^{ww} is independently C₈-C₁₂ alkenyl comprising at least two double bonds (e.g., C₉ or C₁₀ alkenyl comprising two double bonds).

In some embodiments, ionizable lipids of the present disclosure have a structure of Formula (X), wherein each R^{ww} is independently (C₁ alkylenyl)-(cyclopropylene-C₆ alkyl) or (C₂ alkylenyl)-(cyclopropylene-C₂ alkyl). In some embodiments, ionizable lipids of the present disclosure have a structure of Formula (X), wherein each R^{ww} is independently (C₁ alkylenyl)-(cyclopropylene-C₆ alkyl). In some embodiments, ionizable lipids of the present disclosure have a structure of Formula (X), wherein each R^{ww} is independently (C₂ alkylenyl)-(cyclopropylene-C₂ alkyl).

In some embodiments, ionizable lipids of the present disclosure have a structure of Formula (X), wherein each R^{ww} is C₄ alkyl. In some embodiments, ionizable lipids of the present disclosure have a structure of Formula (X), wherein each R^{ww} is C₅ alkyl. In some embodiments, ionizable lipids of the present disclosure have a structure of Formula (X), wherein each R^{ww} is C₆ alkyl. In some embodiments, ionizable lipids of the present disclosure have a structure of Formula (X), wherein each R^{ww} is C₇ alkyl. In some embodiments, ionizable lipids of the present disclosure have a structure of Formula (X), wherein each R^{ww} is C₈ alkyl. In some embodiments, ionizable lipids of the present disclosure have a structure of Formula (X), wherein each R^{ww} is C₉ alkyl. In some embodiments, ionizable lipids of the present disclosure have a structure of Formula (X), wherein each R^{ww} is C₁₀ alkyl. In some embodiments, ionizable lipids of the present disclosure have a structure of Formula (X), wherein each R^{ww} is C₁₁ alkyl. In some embodiments, ionizable lipids of the present disclosure have a structure of Formula (X), wherein each R^{ww} is C₁₂ alkyl. In some embodiments, ionizable lipids of the present disclosure have a structure of Formula (X), wherein each R^{ww} is C₁₃ alkyl. In some embodiments, ionizable lipids of the present disclosure have a structure of Formula (X), wherein each R^{ww} is C₁₄ alkyl.

In some embodiments, ionizable lipids of the present disclosure have a structure of Formula (X), wherein each R^{ww} is C₉ alkenyl. In some embodiments, ionizable lipids of the present disclosure have a structure of Formula (X), wherein each R^{ww} is C₁₀ alkenyl. In some embodiments, ionizable lipids of the present disclosure have a structure of Formula (X), wherein each R^{ww} is C₁₁ alkenyl. In some embodiments, ionizable lipids of the present disclosure have a structure of Formula (X), wherein each R^{ww} is C₁₂ alkenyl.

In some embodiments, ionizable lipids of the present disclosure have a structure of Formula (X), wherein each R^{ww} is C₈ alkenyl comprising at least two double bonds. In some embodiments, ionizable lipids of the present disclosure have a structure of Formula (X), wherein each R^{ww} is C₉ alkenyl comprising at least two double bonds. In some embodiments, ionizable lipids of the present disclosure have a structure of Formula (X), wherein each R^{ww} is C₁₀ alkenyl comprising at least two double bonds. In some embodiments, ionizable lipids of the present disclosure have a structure of Formula (X), wherein each R^{ww} is C₁₁ alkenyl comprising at least two double bonds. In some embodiments, ionizable lipids of the present disclosure have a structure of Formula (X), wherein each R^{ww} is C₁₂ alkenyl comprising at least two double bonds. In some embodiments, ionizable lipids of the present disclosure have a structure of Formula (X), wherein each R^{ww} is C₁₃ alkenyl comprising at least two double bonds. In some embodiments, ionizable lipids of the present disclosure have a structure of Formula (X), wherein each R^{ww} is C₁₄ alkenyl comprising at least two double bonds.

In some embodiments, ionizable lipids of the present disclosure have a structure of Formula (X), wherein each R^{ww} is C₉ alkyl, wherein one-(CH₂)₂— of the C₉ alkyl is replaced with C₂-C₆ cycloalkylenyl. In some embodiments, ionizable lipids of the present disclosure have a structure of Formula (X), wherein each R^{ww} is C₉ alkyl, wherein one-(CH₂)₂— of the C₉ alkyl is replaced with cyclopropylene. In some embodiments, ionizable lipids of the present disclosure have a structure of Formula (X), wherein each R^{ww} is C₉ alkyl, wherein two-(CH₂)₂— of the C₉ alkyl are replaced with C₂-C₆ cycloalkylenyl. In some embodiments, ionizable lipids of the present disclosure have a structure of Formula (X), wherein each R^{ww} is C₉ alkyl, wherein two-(CH₂)₂— of the C₉ alkyl are replaced with cyclopropylene.

In some embodiments, ionizable lipids of the present disclosure have a structure of Formula (X), wherein each R^{ww} is linear C₄ alkyl. In some embodiments, ionizable lipids of the present disclosure have a structure of Formula (X), wherein each R^{ww} is linear C₅ alkyl. In some embodiments, ionizable lipids of the present disclosure have a structure of Formula (X), wherein each R^{ww} is linear C₆ alkyl. In some embodiments, ionizable lipids of the present disclosure have a structure of Formula (X), wherein each R^{ww} is linear C₇ alkyl. In some embodiments, ionizable lipids of the present disclosure have a structure of Formula (X), wherein each R^{ww} is linear C₈ alkyl. In some embodiments, ionizable lipids of the present disclosure have a structure of Formula (X), wherein each R^{ww} is linear C₉ alkyl. In some embodiments, ionizable lipids of the present disclosure have a structure of Formula (X), wherein each R^{ww} is linear C₁₀ alkyl. In some embodiments, ionizable lipids of the present disclosure have a structure of Formula (X), wherein each R^{ww} is linear C₁₁ alkyl. In some embodiments, ionizable lipids of the present disclosure have a structure of Formula (X), wherein each R^{ww} is linear C₁₂ alkyl. In some embodiments, ionizable lipids of the present disclosure have a structure of Formula (X), wherein each R^{ww} is linear C₁₃ alkyl. In some embodiments, ionizable lipids of the present disclosure have a structure of Formula (X), wherein each R^{ww} is linear C₁₄ alkyl.

In some embodiments, ionizable lipids of the present disclosure have a structure of Formula (X), wherein each R^{ww} is branched C₈ alkenyl. In some embodiments, ionizable lipids of the present disclosure have a structure of Formula (X), wherein each R^{ww} is branched C₉ alkenyl. In some embodiments, ionizable lipids of the present disclosure have a structure of Formula (X), wherein each R^{ww} is branched C₁₀ alkenyl. In some embodiments, ionizable lipids of the present disclosure have a structure of Formula (X), wherein each R^{ww} is branched C₁₁ alkenyl. In some embodiments, ionizable lipids of the present disclosure have a structure of Formula (X), wherein each R^{ww} is branched C₁₂ alkenyl.

In some embodiments, ionizable lipids of the present disclosure have a structure of Formula (X), wherein each cc is independently selected from 3 to 7. In some embodiments, ionizable lipids of the present disclosure have a structure of Formula (X), wherein each cc is 3. In some embodiments, ionizable lipids of the present disclosure have a structure of Formula (X), wherein each cc is 4. In some embodiments, ionizable lipids of the present disclosure have a structure of Formula (X), wherein each cc is 5. In some embodiments, ionizable lipids of the present disclosure have a structure of Formula (X), wherein each cc is 6. In some embodiments, ionizable lipids of the present disclosure have a structure of Formula (X), wherein each cc is 7. In some embodiments, ionizable lipids of the present disclosure have a structure of Formula (X), wherein each cc is 8. In some embodiments, ionizable lipids of the present disclosure have a structure of Formula (X), wherein each cc is 9.

In some embodiments, ionizable lipids of the present disclosure have a structure of Formula (X), wherein each dd is independently selected from 1 to 4. In some embodiments, ionizable lipids of the present disclosure have a structure of Formula (X), wherein each dd is 1. In some embodiments, ionizable lipids of the present disclosure have a structure of Formula (X), wherein each dd is 2. In some embodiments, ionizable lipids of the present disclosure have a structure of Formula (X), wherein each dd is 3. In some embodiments, ionizable lipids of the present disclosure have a structure of Formula (X), wherein each dd is 4.

In some embodiments, ionizable lipids of the present disclosure have a structure of Formula (X), wherein ee is 1.

In some embodiments, ionizable lipids of the present disclosure have a structure of Formula (X), wherein ee is 0.

### Formula (X-A)

In some embodiments, ionizable lipids of the present disclosure have a structure of Formula (X), wherein the ionizable lipids of the present disclosure have a structure of Formula (X-A):

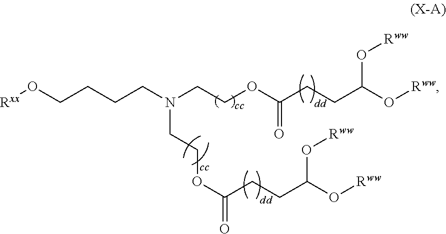

or a pharmaceutically acceptable salt thereof, wherein
each cc is independently selected from 3 to 7;
   each dd is independently selected from 1 to 4;
   R^{xx} is selected from hydrogen and optionally substituted C₁-C₆ alkyl; and
   each R^{ww} is independently selected from the group consisting of C₄-C₁₄ alkyl or (linear or branched C₃-C₅ alkylenyl)-(branched C₅-C₇alkenyl).

In some embodiments, ionizable lipids of the present disclosure have a structure of Formula (X-A), wherein R^{xx} is hydrogen. In some embodiments, ionizable lipids of the present disclosure have a structure of Formula (X-A), wherein R^{xx} is C₁ alkyl. In some embodiments, ionizable lipids of the present disclosure have a structure of Formula (X-A), wherein R^{xx} is C₂ alkyl. In some embodiments, ionizable lipids of the present disclosure have a structure of Formula (X-A), wherein R^{xx} is C₃ alkyl. In some embodiments, ionizable lipids of the present disclosure have a structure of Formula (X-A), wherein R^{xx} is C₄ alkyl. In some embodiments, ionizable lipids of the present disclosure have a structure of Formula (X-A), wherein R^{xx} is C₅ alkyl. In some embodiments, ionizable lipids of the present disclosure have a structure of Formula (X-A), wherein R^{xx} is C₆ alkyl.

In some embodiments, ionizable lipids of the present disclosure have a structure of Formula (X-A), wherein each cc is 4, 5, 6, or 7. In some embodiments, ionizable lipids of the present disclosure have a structure of Formula (X-A), wherein each cc is 3. In some embodiments, ionizable lipids of the present disclosure have a structure of Formula (X-A), wherein each cc is 4. In some embodiments, ionizable lipids of the present disclosure have a structure of Formula (X-A), wherein each cc is 5. In some embodiments, ionizable lipids of the present disclosure have a structure of Formula (X-A), wherein each cc is 6. In some embodiments, ionizable lipids of the present disclosure have a structure of Formula (X-A), wherein each cc is 7.

In some embodiments, ionizable lipids of the present disclosure have a structure of Formula (X-A), wherein each dd is 1 or 3. In some embodiments, ionizable lipids of the present disclosure have a structure of Formula (X-A), wherein each dd is 1. In some embodiments, ionizable lipids of the present disclosure have a structure of Formula (X-A), wherein each dd is 2. In some embodiments, ionizable lipids of the present disclosure have a structure of Formula (X-A), wherein each dd is 3. In some embodiments, ionizable lipids of the present disclosure have a structure of Formula (X-A), wherein each dd is 4.

In some embodiments, ionizable lipids of the present disclosure have a structure of Formula (X-A), wherein each R^{ww} is C₄-C₁₄ alkyl. In some embodiments, ionizable lipids of the present disclosure have a structure of Formula (X-A), wherein each R^{ww} is C₄ alkyl. In some embodiments, ionizable lipids of the present disclosure have a structure of Formula (X-A), wherein each R^{ww} is C₅ alkyl. In some embodiments, ionizable lipids of the present disclosure have a structure of Formula (X-A), wherein each R^{ww} is C₆ alkyl. In some embodiments, ionizable lipids of the present disclosure have a structure of Formula (X-A), wherein each R^{ww} is C₇ alkyl. In some embodiments, ionizable lipids of the present disclosure have a structure of Formula (X-A), wherein each R^{ww} is C₈ alkyl. In some embodiments, ionizable lipids of the present disclosure have a structure of Formula (X-A), wherein each R^{ww} is C₉ alkyl. In some embodiments, ionizable lipids of the present disclosure have a structure of Formula (X-A), wherein each R^{ww} is C₁₀ alkyl. In some embodiments, ionizable lipids of the present disclosure have a structure of Formula (X-A), wherein each R^{ww} is C₁₁ alkyl. In some embodiments, ionizable lipids of the present disclosure have a structure of Formula (X-A), wherein each R^{ww} is C₁₂ alkyl. In some embodiments, ionizable lipids of the present disclosure have a structure of Formula (X-A), wherein each R^{ww} is C₁₃ alkyl. In some embodiments, ionizable lipids of the present disclosure have a structure of Formula (X-A), wherein each R^{ww} is C₁₄ alkyl.

In some embodiments, ionizable lipids of the present disclosure have a structure of Formula (X-A), wherein each R^{ww} is (linear or branched C₃-C₅ alkylenyl)-(branched C₅-C₇alkenyl), e.g., (branched C₅ alkylenyl)-(branched C₅alkenyl), e.g.,

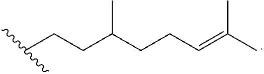 [SMILES: *CCC(C)CCC=C(C)C]

In some embodiments, ionizable lipids of the present disclosure comprise an acyclic core. In some embodiments, ionizable lipids of the present disclosure are selected from any lipid in Table (I) below or a pharmaceutically acceptable salt thereof:

TABLE I
Non-Limiting Examples of Ionizable Lipids with an Acyclic Core

| Structure | # |
|---|---|
| 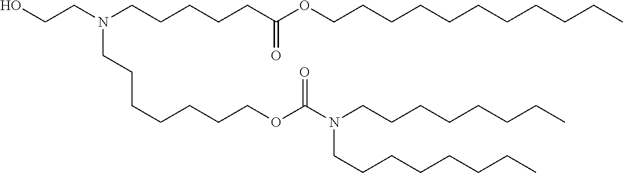 [SMILES: CCCCCCCCCCCOC(=O)CCCCCN(CCO)CCCCCCCOC(=O)N(CCCCCCCC)CCCCCCCC] | 1 |
| 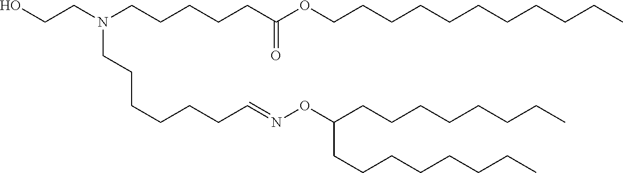 [SMILES: CCCCCCCCCCCOC(=O)CCCCCN(CCO)CCCCCC/C=N/OC(CCCCCCCC)CCCCCCCC] | 2 |
| 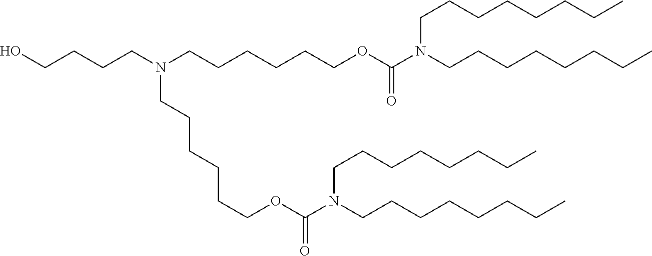 [SMILES: CCCCCCCCN(CCCCCCCC)C(=O)OCCCCCCN(CCCCO)CCCCCCOC(=O)N(CCCCCCCC)CCCCCCCC] | 3 |
| 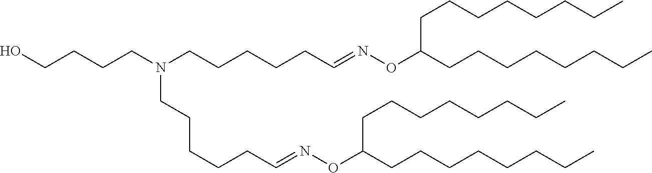 [SMILES: CCCCCCCCC(CCCCCCCC)O/N=C/CCCCCN(CCCCO)CCCCC/C=N/OC(CCCCCCCC)CCCCCCCC] | 4 |
| 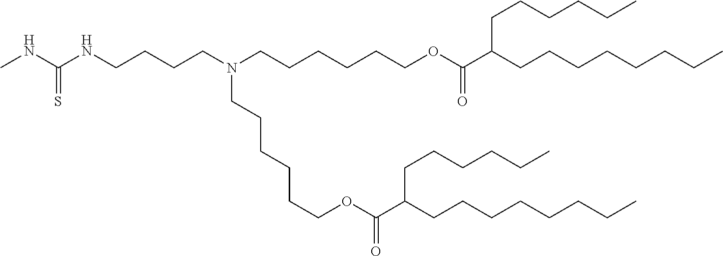 [SMILES: CCCCCCCCC(CCCCCC)C(=O)OCCCCCCN(CCCCCCOC(=O)C(CCCCCC)CCCCCCCC)CCCCNC(=S)NC] | 5 |
| 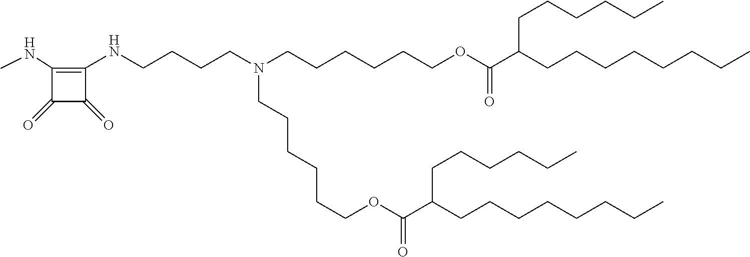 [SMILES: CCCCCCCCC(CCCCCC)C(=O)OCCCCCCN(CCCCCCOC(=O)C(CCCCCC)CCCCCCCC)CCCCNc1c(NC)c(=O)c1=O] | 6 |
| 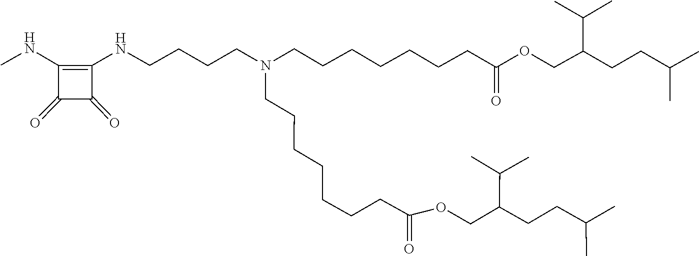 [SMILES: CNc1c(NCCCCN(CCCCCCCC(=O)OCC(CCC(C)C)C(C)C)CCCCCCCC(=O)OCC(CCC(C)C)C(C)C)c(=O)c1=O] | 7 |
| 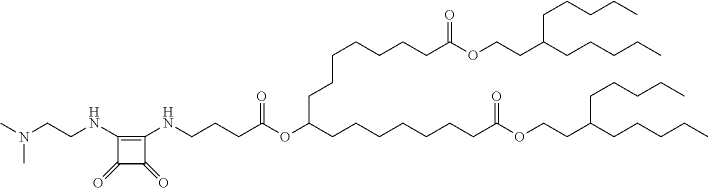 [SMILES: CCCCCC(CCCCC)CCOC(=O)CCCCCCCC(CCCCCCCC(=O)OCCC(CCCCC)CCCCC)OC(=O)CCCNc1c(NCCN(C)C)c(=O)c1=O] | 8 |
| 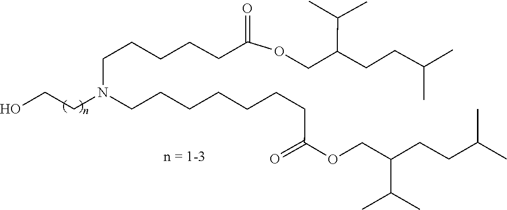 | 9 |
| 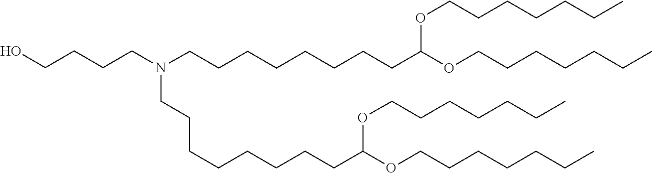 [SMILES: CCCCCCCOC(CCCCCCCCN(CCCCO)CCCCCCCCC(OCCCCCCC)OCCCCCCC)OCCCCCCC] | 10 |
| 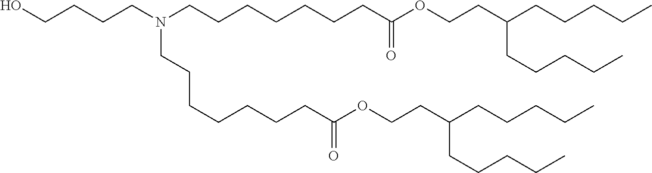 [SMILES: CCCCCC(CCCCC)CCOC(=O)CCCCCCCN(CCCCO)CCCCCCCC(=O)OCCC(CCCCC)CCCCC] | 11 |
| 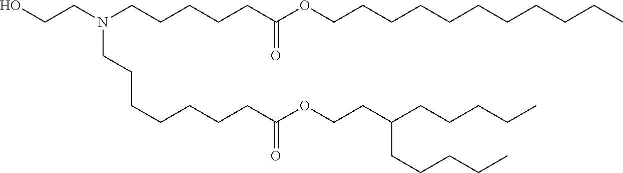 [SMILES: CCCCCCCCCCCOC(=O)CCCCCN(CCO)CCCCCCCC(=O)OCCC(CCCCC)CCCCC] | 13 |
| 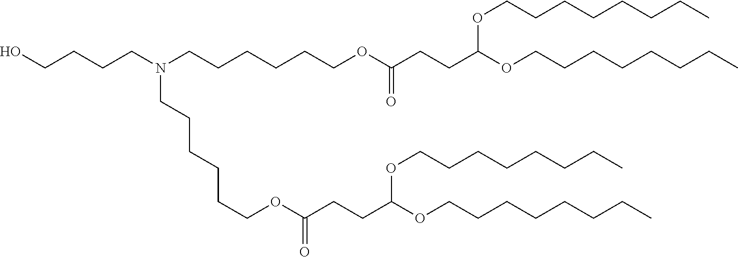 [SMILES: CCCCCCCCOC(CCC(=O)OCCCCCCN(CCCCO)CCCCCCOC(=O)CCC(OCCCCCCCC)OCCCCCCCC)OCCCCCCCC] | 14 |
| 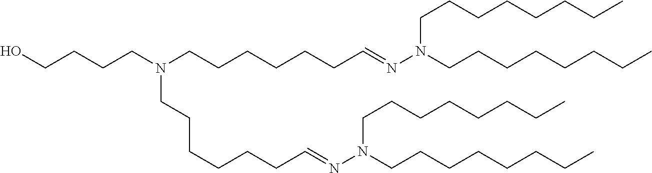 [SMILES: CCCCCCCCN(CCCCCCCC)/N=C/CCCCCCN(CCCCO)CCCCCC/C=N/N(CCCCCCCC)CCCCCCCC] | 15 |
| 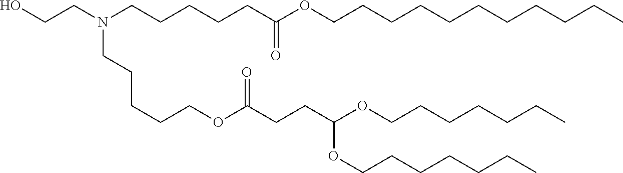 [SMILES: CCCCCCCCCCCOC(=O)CCCCCN(CCO)CCCCCOC(=O)CCC(OCCCCCCC)OCCCCCCC] | 16 |
| 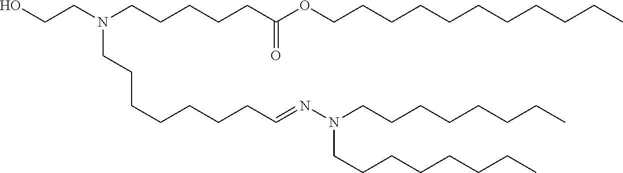 [SMILES: CCCCCCCCCCCOC(=O)CCCCCN(CCO)CCCCCCC/C=N/N(CCCCCCCC)CCCCCCCC] | 17 |
| 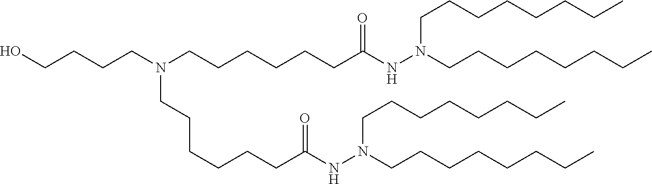 [SMILES: CCCCCCCCN(CCCCCCCC)NC(=O)CCCCCCN(CCCCO)CCCCCCC(=O)NN(CCCCCCCC)CCCCCCCC] | 18 |
| 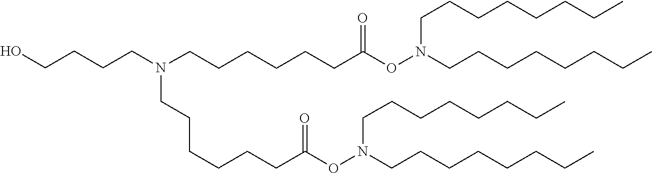 [SMILES: CCCCCCCCN(CCCCCCCC)OC(=O)CCCCCCN(CCCCO)CCCCCCC(=O)ON(CCCCCCCC)CCCCCCCC] | 19 |
| 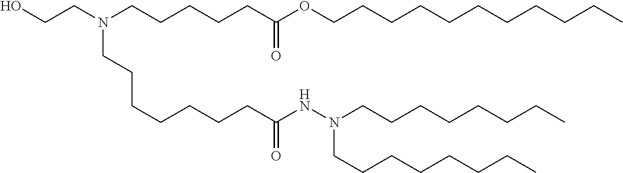 [SMILES: CCCCCCCCCCCOC(=O)CCCCCN(CCO)CCCCCCCC(=O)NN(CCCCCCCC)CCCCCCCC] | 20 |
| 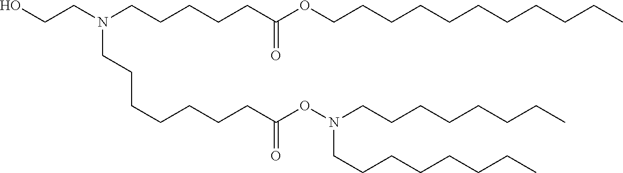 [SMILES: CCCCCCCCCCCOC(=O)CCCCCN(CCO)CCCCCCCC(=O)ON(CCCCCCCC)CCCCCCCC] | 21 |
| 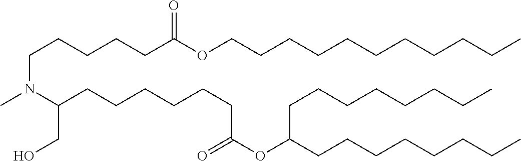 [SMILES: CCCCCCCCCCCOC(=O)CCCCCN(C)C(CO)CCCCCCC(=O)OC(CCCCCCCC)CCCCCCCC] | 22 |
| 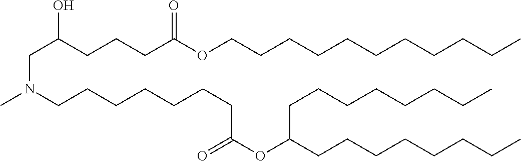 [SMILES: CCCCCCCCCCCOC(=O)CCCC(O)CN(C)CCCCCCCC(=O)OC(CCCCCCCC)CCCCCCCC] | 23 |
| 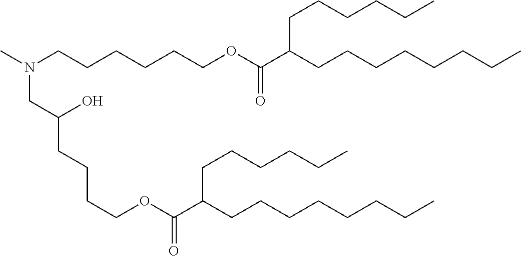 [SMILES: CCCCCCCCC(CCCCCC)C(=O)OCCCCCCN(C)CC(O)CCCCOC(=O)C(CCCCCC)CCCCCCCC] | 24 |
| 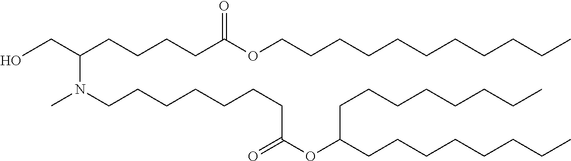 | 25 |
| 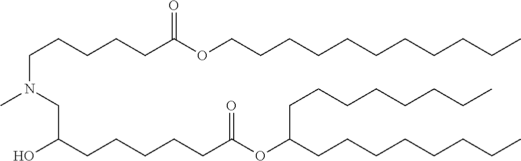 [SMILES: CCCCCCCCCCCOC(=O)CCCCCN(C)CC(O)CCCCCC(=O)OC(CCCCCCCC)CCCCCCCC] | 26 |
| 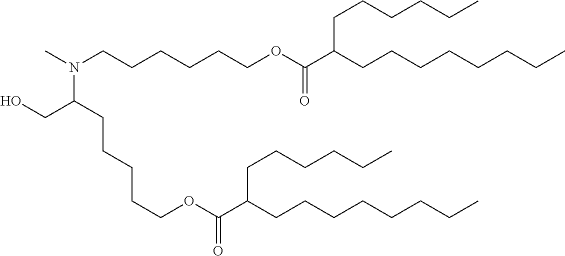 [SMILES: CCCCCCCCC(CCCCCC)C(=O)OCCCCCCN(C)C(CO)CCCCCOC(=O)C(CCCCCC)CCCCCCCC] | 27 |
| 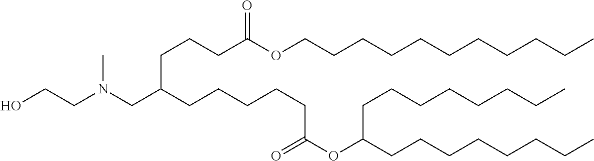 [SMILES: CCCCCCCCCCCOC(=O)CCCC(CCCCCC(=O)OC(CCCCCCCC)CCCCCCCC)CN(C)CCO] | 28 |
| 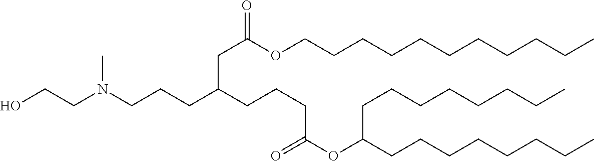 [SMILES: CCCCCCCCCCCOC(=O)CC(CCCC(=O)OC(CCCCCCCC)CCCCCCCC)CCCN(C)CCO] | 29 |
| 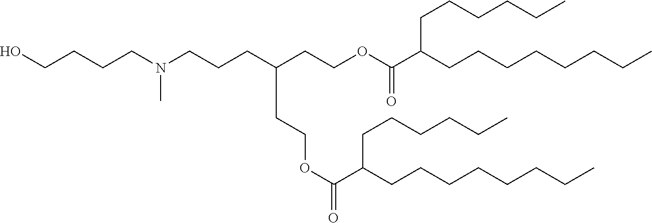 [SMILES: CCCCCCCCC(CCCCCC)C(=O)OCCC(CCCN(C)CCCCO)CCOC(=O)C(CCCCCC)CCCCCCCC] | 30 |
| 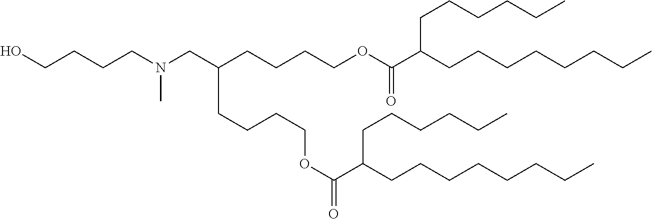 [SMILES: CCCCCCCCC(CCCCCC)C(=O)OCCCCC(CCCCOC(=O)C(CCCCCC)CCCCCCCC)CN(C)CCCCO] | 31 |
| 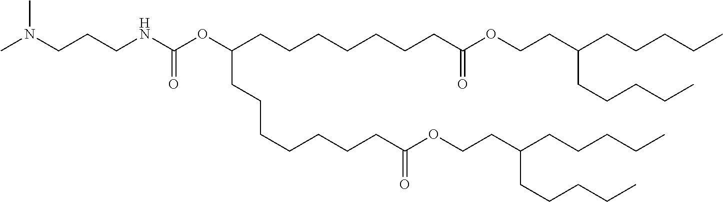 [SMILES: CCCCCC(CCCCC)CCOC(=O)CCCCCCCC(CCCCCCCC(=O)OCCC(CCCCC)CCCCC)OC(=O)NCCCN(C)C] | 32 |
| 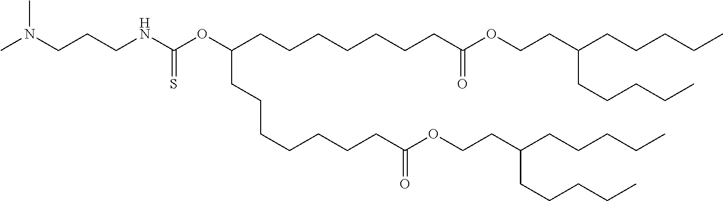 [SMILES: CCCCCC(CCCCC)CCOC(=O)CCCCCCCC(CCCCCCCC(=O)OCCC(CCCCC)CCCCC)OC(=S)NCCCN(C)C] | 33 |
| 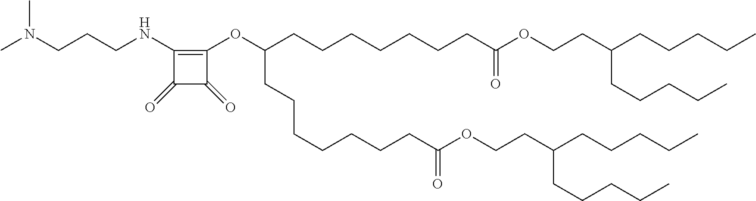 [SMILES: CCCCCC(CCCCC)CCOC(=O)CCCCCCCC(CCCCCCCC(=O)OCCC(CCCCC)CCCCC)Oc1c(NCCCN(C)C)c(=O)c1=O] | 34 |
| 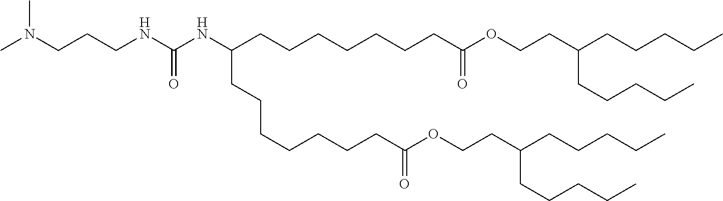 [SMILES: CCCCCC(CCCCC)CCOC(=O)CCCCCCCC(CCCCCCCC(=O)OCCC(CCCCC)CCCCC)NC(=O)NCCCN(C)C] | 35 |
| 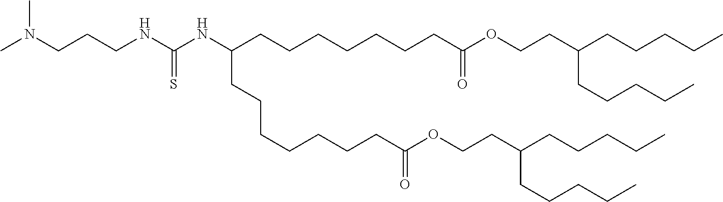 [SMILES: CCCCCC(CCCCC)CCOC(=O)CCCCCCCC(CCCCCCCC(=O)OCCC(CCCCC)CCCCC)NC(=S)NCCCN(C)C] | 36 |
| 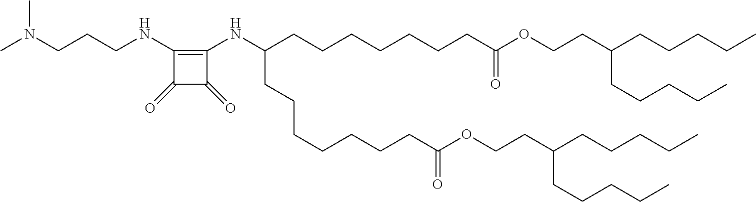 [SMILES: CCCCCC(CCCCC)CCOC(=O)CCCCCCCC(CCCCCCCC(=O)OCCC(CCCCC)CCCCC)Nc1c(NCCCN(C)C)c(=O)c1=O] | 37 |
| 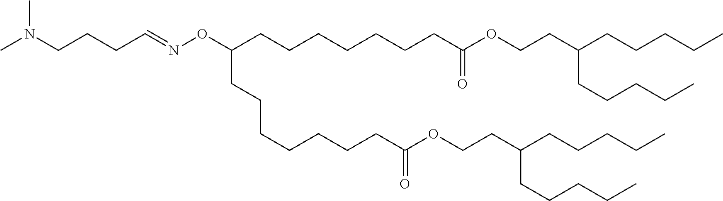 [SMILES: CCCCCC(CCCCC)CCOC(=O)CCCCCCCC(CCCCCCCC(=O)OCCC(CCCCC)CCCCC)O/N=C/CCCN(C)C] | 38 |
| 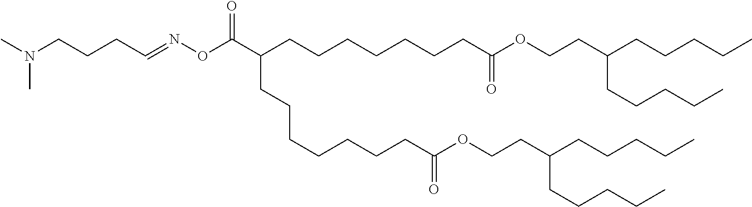 [SMILES: CCCCCC(CCCCC)CCOC(=O)CCCCCCCC(CCCCCCCC(=O)OCCC(CCCCC)CCCCC)C(=O)O/N=C/CCCN(C)C] | 39 |
| 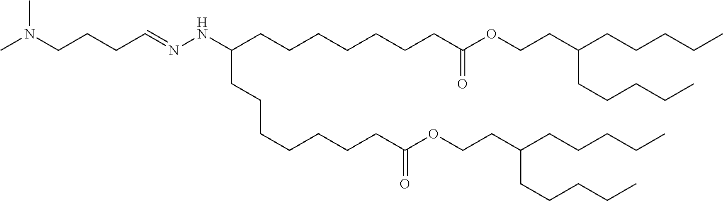 [SMILES: CCCCCC(CCCCC)CCOC(=O)CCCCCCCC(CCCCCCCC(=O)OCCC(CCCCC)CCCCC)N/N=C/CCCN(C)C] | 40 |
| 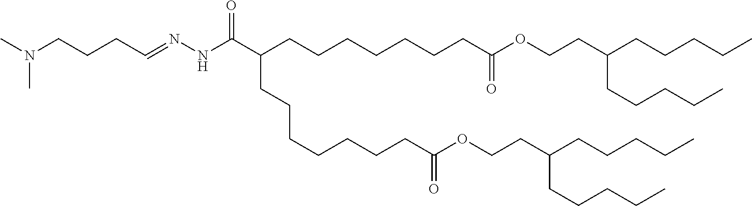 [SMILES: CCCCCC(CCCCC)CCOC(=O)CCCCCCCC(CCCCCCCC(=O)OCCC(CCCCC)CCCCC)C(=O)N/N=C/CCCN(C)C] | 41 |
| 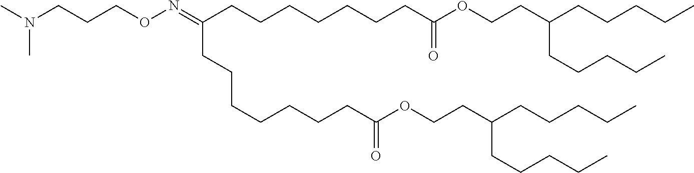 [SMILES: CCCCCC(CCCCC)CCOC(=O)CCCCCCCC(CCCCCCCC(=O)OCCC(CCCCC)CCCCC)=NOCCCN(C)C] | 42 |
| 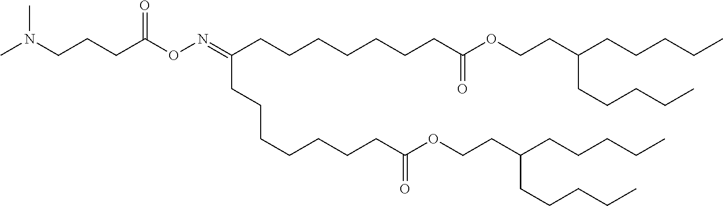 [SMILES: CCCCCC(CCCCC)CCOC(=O)CCCCCCCC(CCCCCCCC(=O)OCCC(CCCCC)CCCCC)=NOC(=O)CCCN(C)C] | 43 |
| 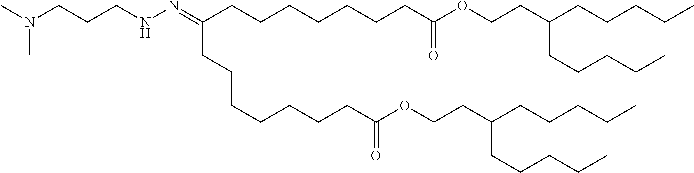 [SMILES: CCCCCC(CCCCC)CCOC(=O)CCCCCCCC(CCCCCCCC(=O)OCCC(CCCCC)CCCCC)=NNCCCN(C)C] | 44 |
| 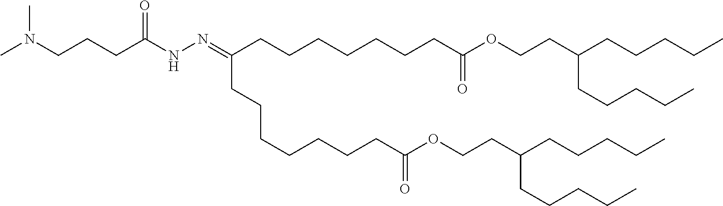 [SMILES: CCCCCC(CCCCC)CCOC(=O)CCCCCCCC(CCCCCCCC(=O)OCCC(CCCCC)CCCCC)=NNC(=O)CCCN(C)C] | 45 |
| 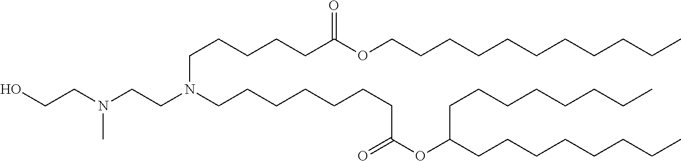 [SMILES: CCCCCCCCCCCOC(=O)CCCCCN(CCCCCCCC(=O)OC(CCCCCCCC)CCCCCCCC)CCN(C)CCO] | 46 |
| 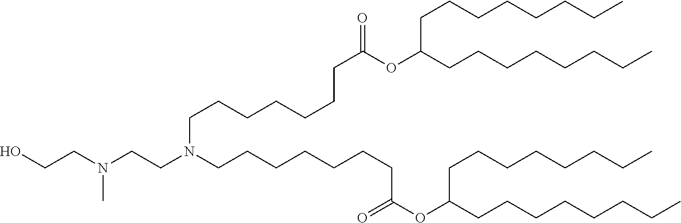 [SMILES: CCCCCCCCC(CCCCCCCC)OC(=O)CCCCCCCN(CCCCCCCC(=O)OC(CCCCCCCC)CCCCCCCC)CCN(C)CCO] | 47 |
| 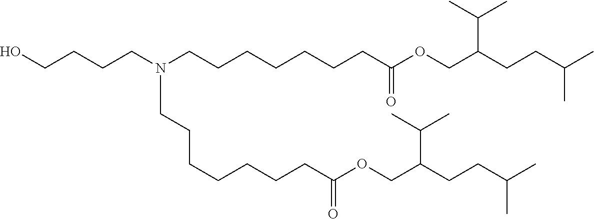 [SMILES: CC(C)CCC(COC(=O)CCCCCCCN(CCCCO)CCCCCCCC(=O)OCC(CCC(C)C)C(C)C)C(C)C] | 48 |
| 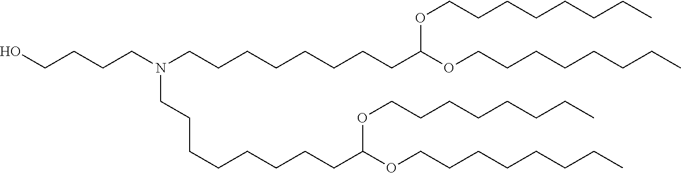 [SMILES: CCCCCCCCOC(CCCCCCCCN(CCCCO)CCCCCCCCC(OCCCCCCCC)OCCCCCCCC)OCCCCCCCC] | 49 |
| 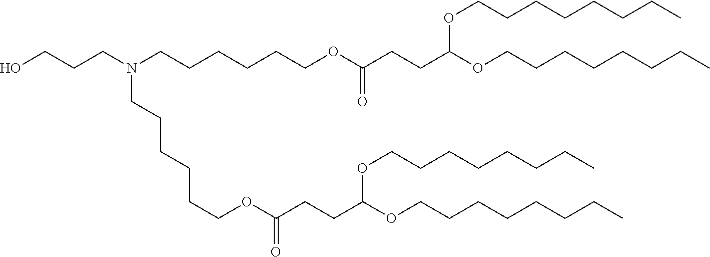 [SMILES: CCCCCCCCOC(CCC(=O)OCCCCCCN(CCCO)CCCCCCOC(=O)CCC(OCCCCCCCC)OCCCCCCCC)OCCCCCCCC] | 50 |
| 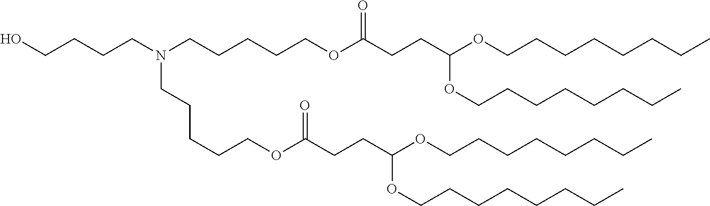 [SMILES: CCCCCCCCOC(CCC(=O)OCCCCCN(CCCCO)CCCCCOC(=O)CCC(OCCCCCCCC)OCCCCCCCC)OCCCCCCCC] | 51 |
| 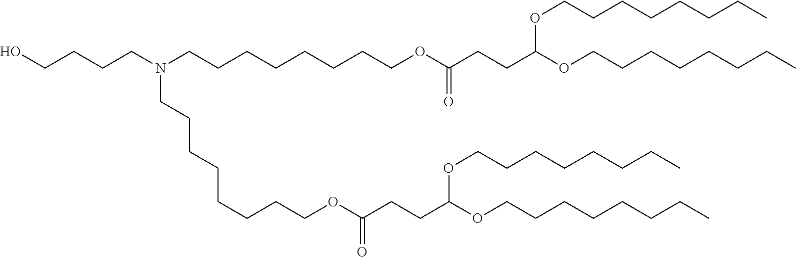 [SMILES: CCCCCCCCOC(CCC(=O)OCCCCCCCCN(CCCCO)CCCCCCCCOC(=O)CCC(OCCCCCCCC)OCCCCCCCC)OCCCCCCCC] | 52 |
| 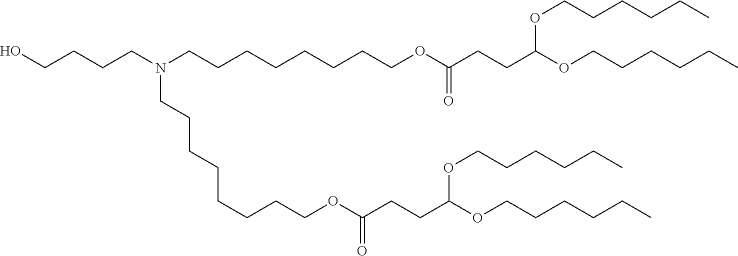 [SMILES: CCCCCCOC(CCC(=O)OCCCCCCCCN(CCCCO)CCCCCCCCOC(=O)CCC(OCCCCCC)OCCCCCC)OCCCCCC] | 53 |
| 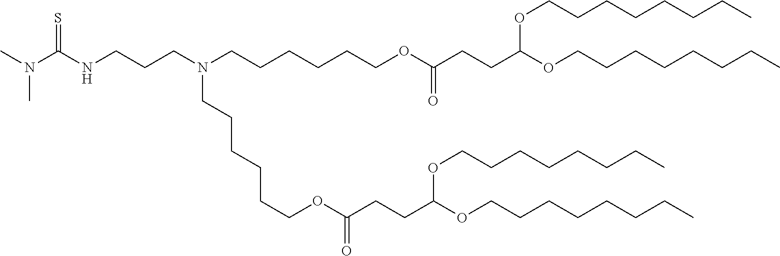 [SMILES: CCCCCCCCOC(CCC(=O)OCCCCCCN(CCCCCCOC(=O)CCC(OCCCCCCCC)OCCCCCCCC)CCCNC(=S)N(C)C)OCCCCCCCC] | 54 |
| 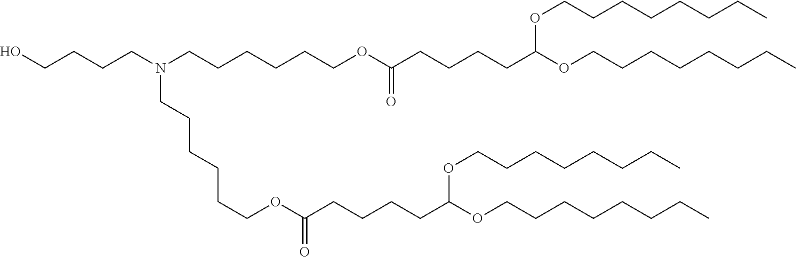 [SMILES: CCCCCCCCOC(CCCCC(=O)OCCCCCCN(CCCCO)CCCCCCOC(=O)CCCCC(OCCCCCCCC)OCCCCCCCC)OCCCCCCCC] | 55 |
| 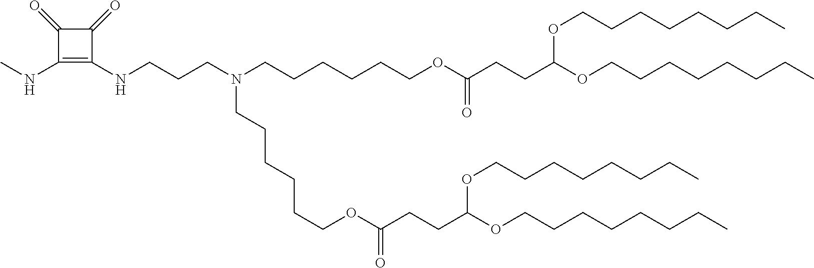 [SMILES: CCCCCCCCOC(CCC(=O)OCCCCCCN(CCCCCCOC(=O)CCC(OCCCCCCCC)OCCCCCCCC)CCCNc1c(NC)c(=O)c1=O)OCCCCCCCC] | 56 |
| 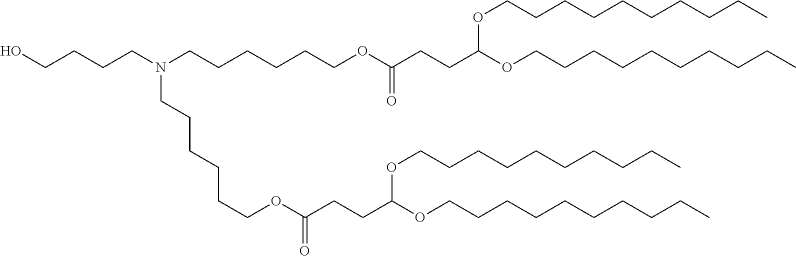 [SMILES: CCCCCCCCCCOC(CCC(=O)OCCCCCCN(CCCCO)CCCCCCOC(=O)CCC(OCCCCCCCCCC)OCCCCCCCCCC)OCCCCCCCCCC] | 57 |
| 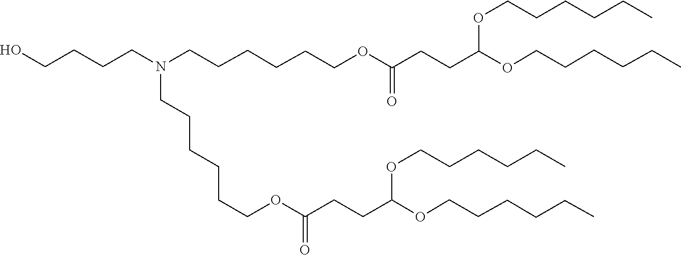 [SMILES: CCCCCCOC(CCC(=O)OCCCCCCN(CCCCO)CCCCCCOC(=O)CCC(OCCCCCC)OCCCCCC)OCCCCCC] | 58 |
| 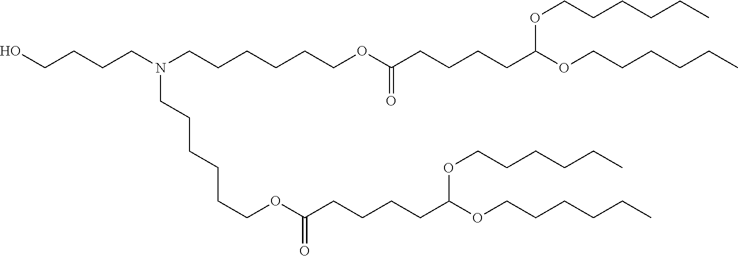 [SMILES: CCCCCCOC(CCCCC(=O)OCCCCCCN(CCCCO)CCCCCCOC(=O)CCCCC(OCCCCCC)OCCCCCC)OCCCCCC] | 59 |
| 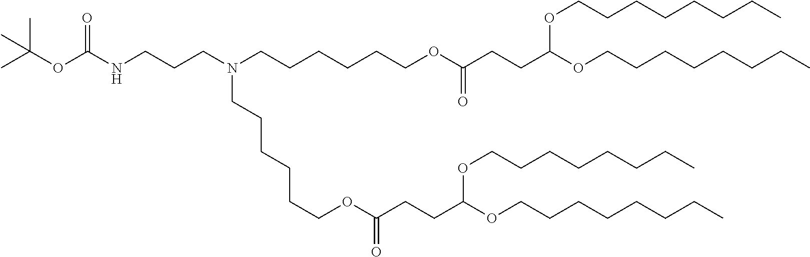 [SMILES: CCCCCCCCOC(CCC(=O)OCCCCCCN(CCCCCCOC(=O)CCC(OCCCCCCCC)OCCCCCCCC)CCCNC(=O)OC(C)(C)C)OCCCCCCCC] | 60 |
| 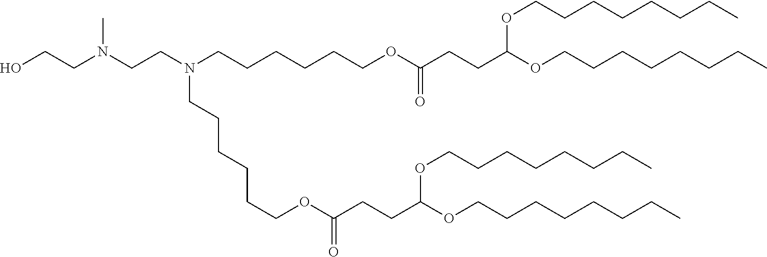 [SMILES: CCCCCCCCOC(CCC(=O)OCCCCCCN(CCCCCCOC(=O)CCC(OCCCCCCCC)OCCCCCCCC)CCN(C)CCO)OCCCCCCCC] | 61 |
| 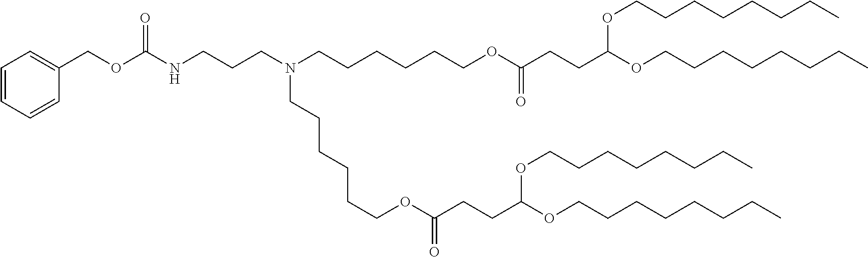 [SMILES: CCCCCCCCOC(CCC(=O)OCCCCCCN(CCCCCCOC(=O)CCC(OCCCCCCCC)OCCCCCCCC)CCCNC(=O)OCc1ccccc1)OCCCCCCCC] | 62 |
| 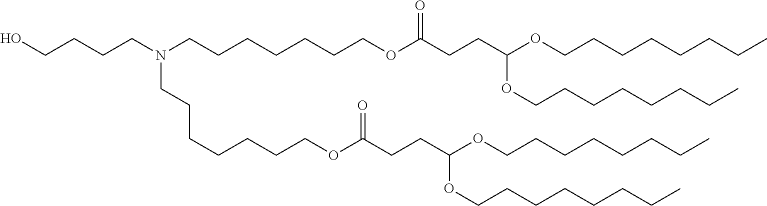 [SMILES: CCCCCCCCOC(CCC(=O)OCCCCCCCN(CCCCO)CCCCCCCOC(=O)CCC(OCCCCCCCC)OCCCCCCCC)OCCCCCCCC] | 64 |
| 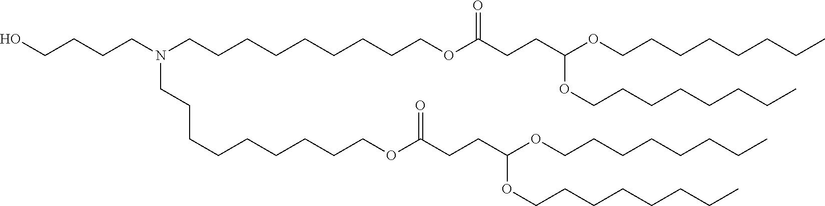 [SMILES: CCCCCCCCOC(CCC(=O)OCCCCCCCCCN(CCCCO)CCCCCCCCCOC(=O)CCC(OCCCCCCCC)OCCCCCCCC)OCCCCCCCC] | 65 |
| 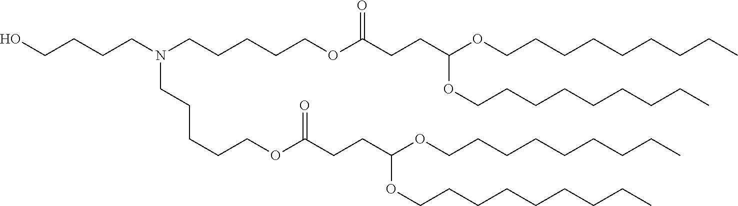 [SMILES: CCCCCCCCCOC(CCC(=O)OCCCCCN(CCCCO)CCCCCOC(=O)CCC(OCCCCCCCCC)OCCCCCCCCC)OCCCCCCCCC] | 66 |
| 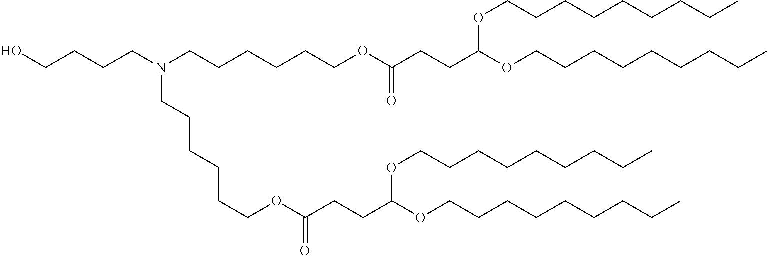 [SMILES: CCCCCCCCCOC(CCC(=O)OCCCCCCN(CCCCO)CCCCCCOC(=O)CCC(OCCCCCCCCC)OCCCCCCCCC)OCCCCCCCCC] | 67 |
| 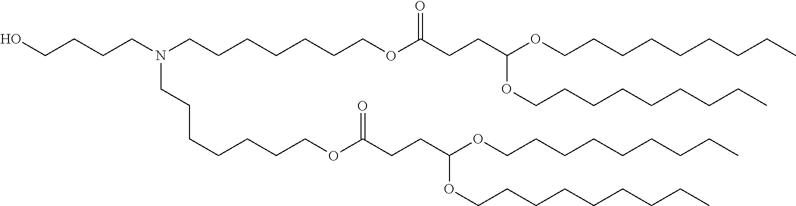 [SMILES: CCCCCCCCCOC(CCC(=O)OCCCCCCCN(CCCCO)CCCCCCCOC(=O)CCC(OCCCCCCCCC)OCCCCCCCCC)OCCCCCCCCC] | 68 |
| 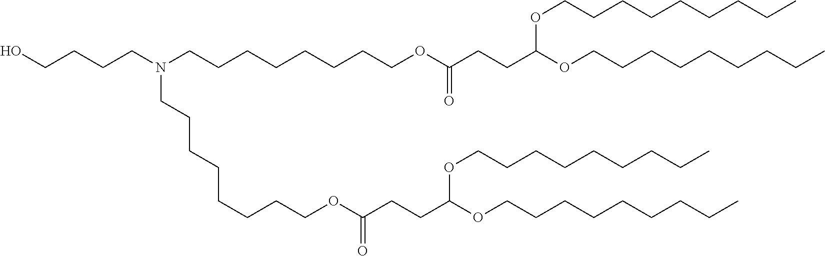 [SMILES: CCCCCCCCCOC(CCC(=O)OCCCCCCCCN(CCCCO)CCCCCCCCOC(=O)CCC(OCCCCCCCCC)OCCCCCCCCC)OCCCCCCCCC] | 69 |
| 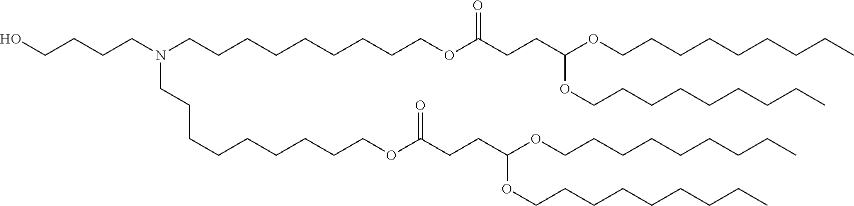 [SMILES: CCCCCCCCCOC(CCC(=O)OCCCCCCCCCN(CCCCO)CCCCCCCCCOC(=O)CCC(OCCCCCCCCC)OCCCCCCCCC)OCCCCCCCCC] | 70 |
| 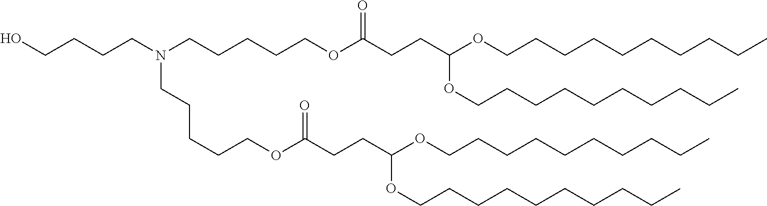 [SMILES: CCCCCCCCCCOC(CCC(=O)OCCCCCN(CCCCO)CCCCCOC(=O)CCC(OCCCCCCCCCC)OCCCCCCCCCC)OCCCCCCCCCC] | 71 |
| 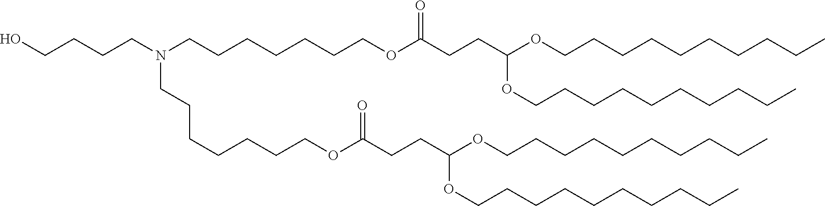 [SMILES: CCCCCCCCCCOC(CCC(=O)OCCCCCCCN(CCCCO)CCCCCCCOC(=O)CCC(OCCCCCCCCCC)OCCCCCCCCCC)OCCCCCCCCCC] | 72 |
| 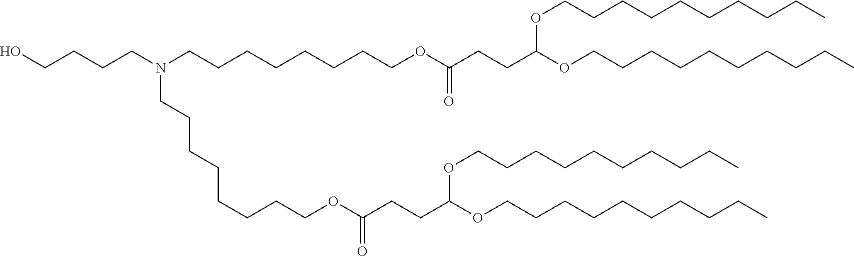 [SMILES: CCCCCCCCCCOC(CCC(=O)OCCCCCCCCN(CCCCO)CCCCCCCCOC(=O)CCC(OCCCCCCCCCC)OCCCCCCCCCC)OCCCCCCCCCC] | 73 |
| 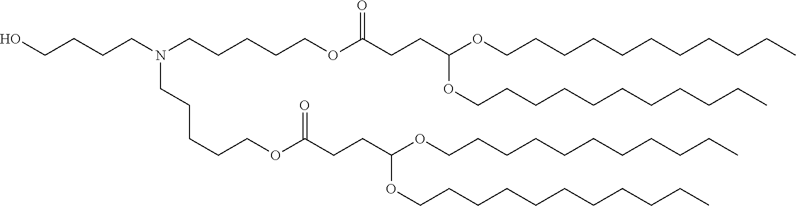 [SMILES: CCCCCCCCCCCOC(CCC(=O)OCCCCCN(CCCCO)CCCCCOC(=O)CCC(OCCCCCCCCCCC)OCCCCCCCCCCC)OCCCCCCCCCCC] | 74 |
| 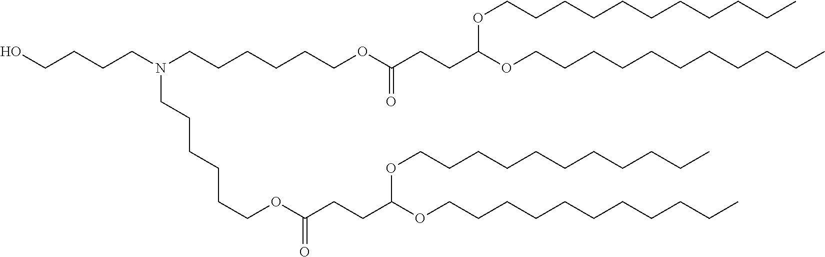 [SMILES: CCCCCCCCCCCOC(CCC(=O)OCCCCCCN(CCCCO)CCCCCCOC(=O)CCC(OCCCCCCCCCCC)OCCCCCCCCCCC)OCCCCCCCCCCC] | 75 |
| 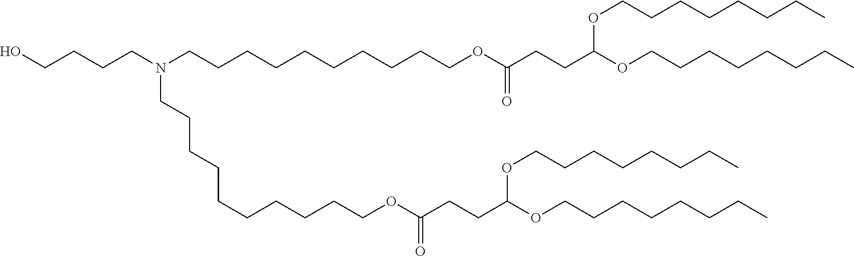 [SMILES: CCCCCCCCOC(CCC(=O)OCCCCCCCCCCN(CCCCO)CCCCCCCCCCOC(=O)CCC(OCCCCCCCC)OCCCCCCCC)OCCCCCCCC] | 76 |
| 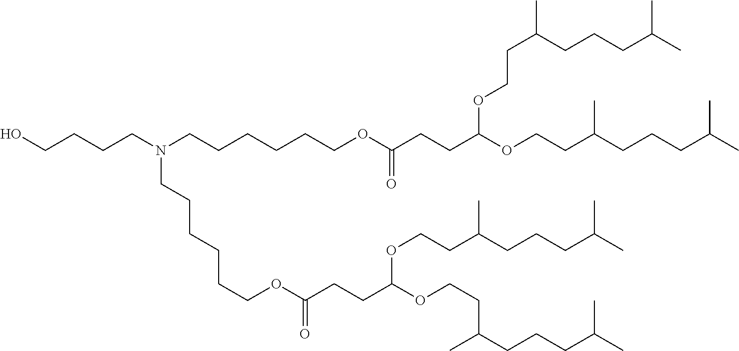 [SMILES: CC(C)CCCC(C)CCOC(CCC(=O)OCCCCCCN(CCCCO)CCCCCCOC(=O)CCC(OCCC(C)CCCC(C)C)OCCC(C)CCCC(C)C)OCCC(C)CCCC(C)C] | 77 |
| 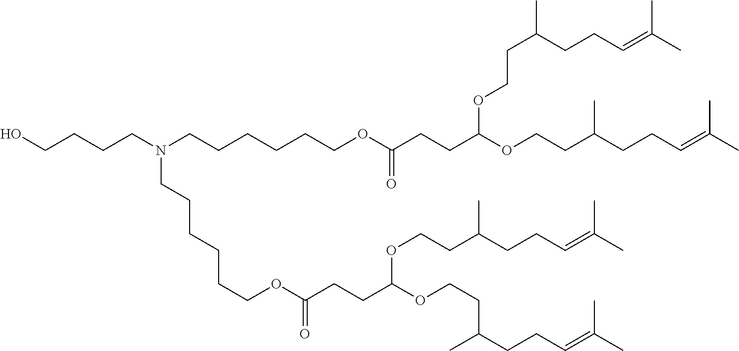 [SMILES: CC(C)=CCCC(C)CCOC(CCC(=O)OCCCCCCN(CCCCO)CCCCCCOC(=O)CCC(OCCC(C)CCC=C(C)C)OCCC(C)CCC=C(C)C)OCCC(C)CCC=C(C)C] | 78 |
| 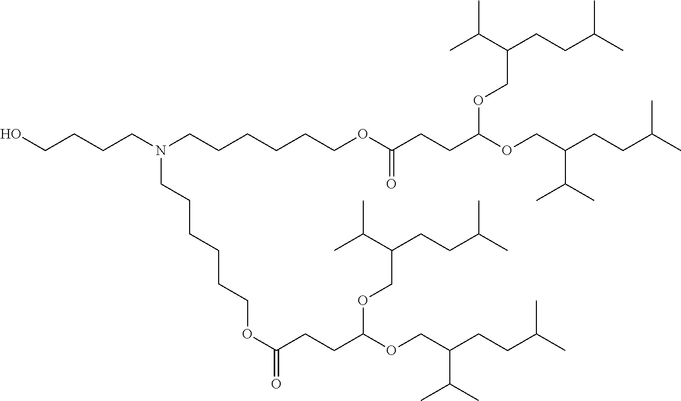 [SMILES: CC(C)CCC(COC(CCC(=O)OCCCCCCN(CCCCO)CCCCCCOC(=O)CCC(OCC(CCC(C)C)C(C)C)OCC(CCC(C)C)C(C)C)OCC(CCC(C)C)C(C)C)C(C)C] | 79 |
| 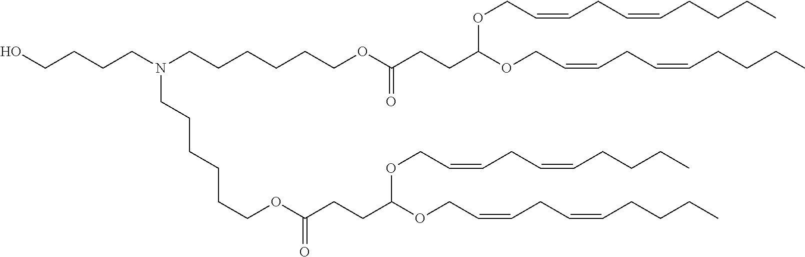 [SMILES: CCCC/C=C\C/C=C\COC(CCC(=O)OCCCCCCN(CCCCO)CCCCCCOC(=O)CCC(OC/C=C\C/C=C\CCCC)OC/C=C\C/C=C\CCCC)OC/C=C\C/C=C\CCCC] | 80 |
| 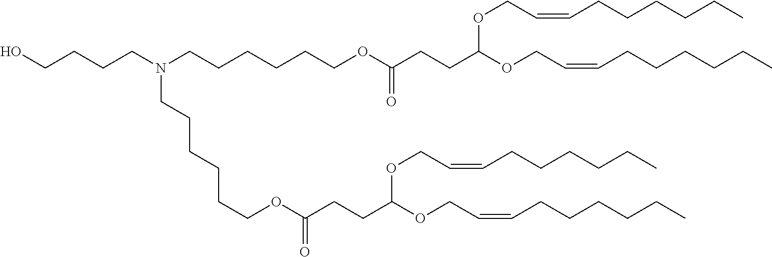 [SMILES: CCCCCC/C=C\COC(CCC(=O)OCCCCCCN(CCCCO)CCCCCCOC(=O)CCC(OC/C=C\CCCCCC)OC/C=C\CCCCCC)OC/C=C\CCCCCC] | 81 |
| 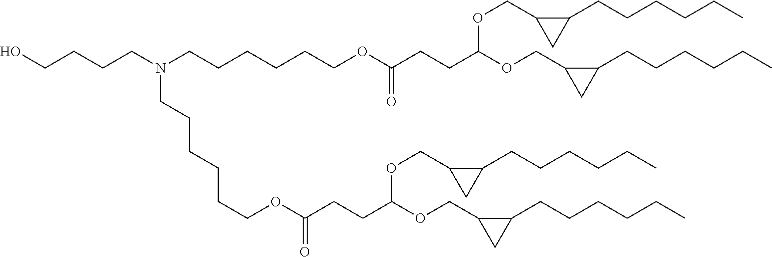 [SMILES: CCCCCCC1CC1COC(CCC(=O)OCCCCCCN(CCCCO)CCCCCCOC(=O)CCC(OCC1CC1CCCCCC)OCC1CC1CCCCCC)OCC1CC1CCCCCC] | 82 |
| 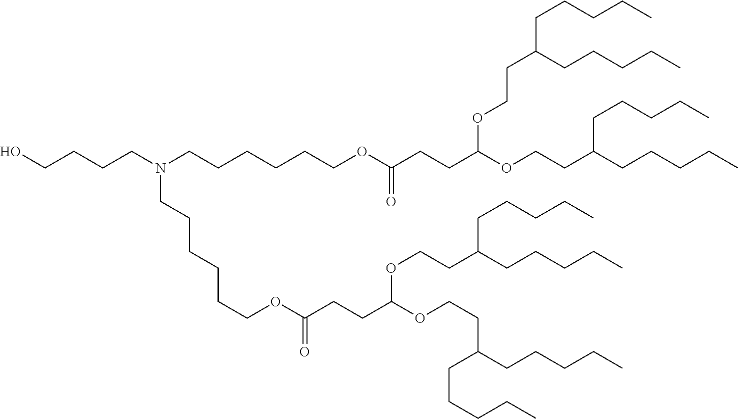 [SMILES: CCCCCC(CCCCC)CCOC(CCC(=O)OCCCCCCN(CCCCO)CCCCCCOC(=O)CCC(OCCC(CCCCC)CCCCC)OCCC(CCCCC)CCCCC)OCCC(CCCCC)CCCCC] | 83 |
| 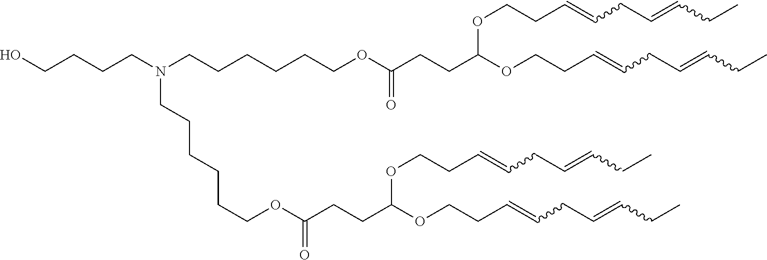 [SMILES: CCC=CCC=CCCOC(CCC(=O)OCCCCCCN(CCCCO)CCCCCCOC(=O)CCC(OCCC=CCC=CCC)OCCC=CCC=CCC)OCCC=CCC=CCC] | 84 |
| 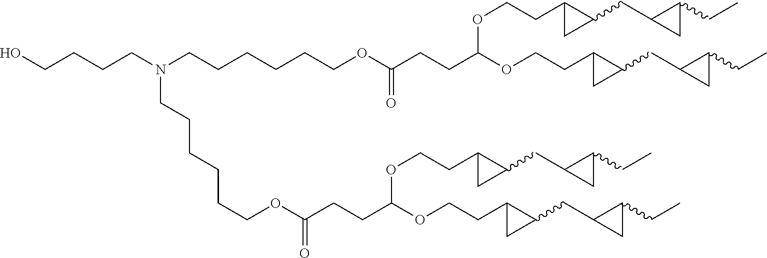 [SMILES: CCC1CC1CC1CC1CCOC(CCC(=O)OCCCCCCN(CCCCO)CCCCCCOC(=O)CCC(OCCC1CC1CC1CC1CC)OCCC1CC1CC1CC1CC)OCCC1CC1CC1CC1CC] | 85 |
| 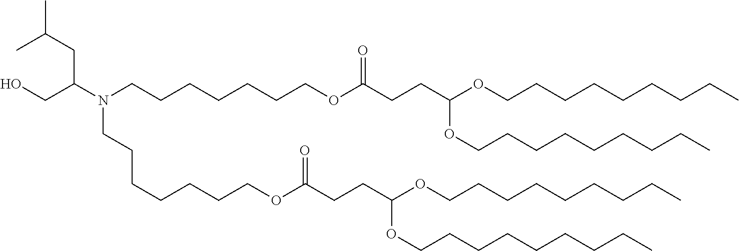 [SMILES: CCCCCCCCCOC(CCC(=O)OCCCCCCCN(CCCCCCCOC(=O)CCC(OCCCCCCCCC)OCCCCCCCCC)C(CO)CC(C)C)OCCCCCCCCC] | 86 |
| 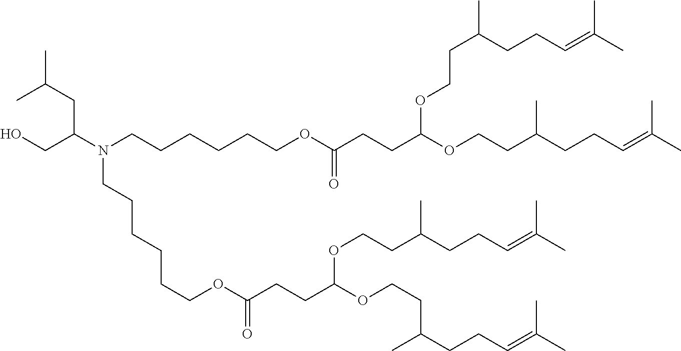 [SMILES: CC(C)=CCCC(C)CCOC(CCC(=O)OCCCCCCN(CCCCCCOC(=O)CCC(OCCC(C)CCC=C(C)C)OCCC(C)CCC=C(C)C)C(CO)CC(C)C)OCCC(C)CCC=C(C)C] | 87 |
| 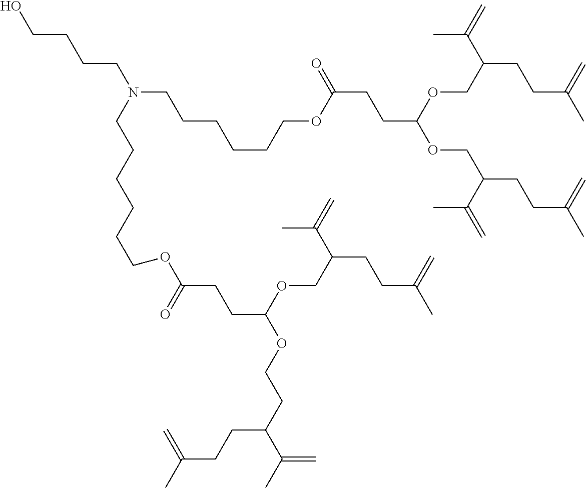 [SMILES: C=C(C)CCC(CCOC(CCC(=O)OCCCCCCN(CCCCO)CCCCCCOC(=O)CCC(OCC(CCC(=C)C)C(=C)C)OCC(CCC(=C)C)C(=C)C)OCC(CCC(=C)C)C(=C)C)C(=C)C] | 88 |
| 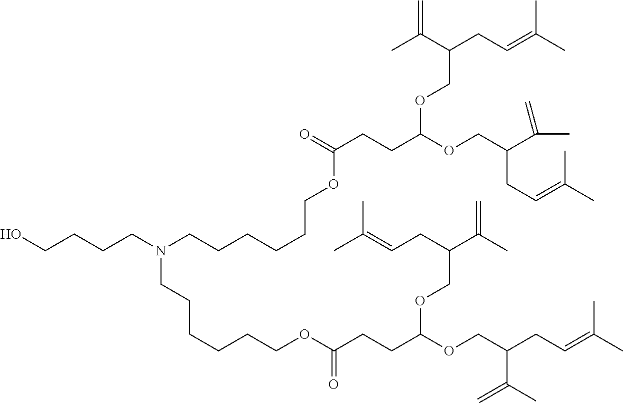 [SMILES: C=C(C)C(CC=C(C)C)COC(CCC(=O)OCCCCCCN(CCCCO)CCCCCCOC(=O)CCC(OCC(CC=C(C)C)C(=C)C)OCC(CC=C(C)C)C(=C)C)OCC(CC=C(C)C)C(=C)C] | 89 |

### Series “CY”

In some embodiments, an LNP of the present disclosure comprises an ionizable lipid disclosed in PCT Application Publication WO2023044333A1, which is incorporated by reference herein, in its entirety.

### Formula (CY)

In some embodiments, an LNP disclosed herein comprises an ionizable lipid of Formula (CY)

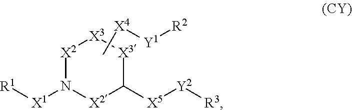

or a pharmaceutically acceptable salt thereof,
wherein:

R¹ is selected from the group consisting of —OH, —OAc, R^{1a},

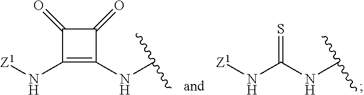

Z¹ is optionally substituted C₁-C₆ alkyl;
X¹ is optionally substituted C₂-C₆ alkylenyl;
X² is selected from the group consisting of a bond, —CH₂— and —CH₂CH₂—;
X^{2′} is selected from the group consisting of a bond, —CH₂— and —CH₂CH₂—;
X³ is selected from the group consisting of a bond, —CH₂— and —CH₂CH₂—;
X^{3′} is selected from the group consisting of a bond, —CH₂— and —CH₂CH₂—;
X⁴ and X⁵ are independently optionally substituted C₂-C₁₄ alkylenyl or optionally substituted C₂-C₁₄ alkenylenyl;
Y¹ and Y² are independently selected from the group consisting of

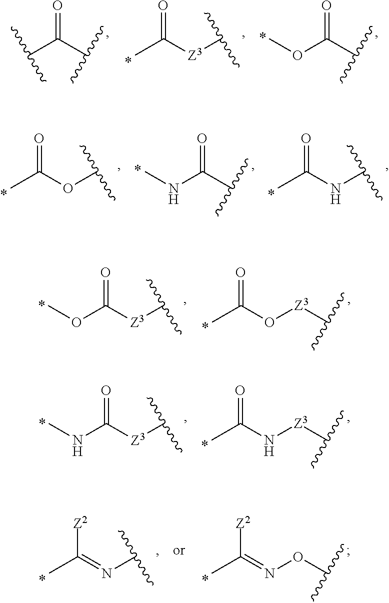

wherein the bond marked with an “*” is attached to X⁴ or X⁵;
each Z² is independently H or optionally substituted C₁-C₈ alkyl;
each Z³ is independently optionally substituted C₁-C₆ alkylenyl;
R² is selected from the group consisting of optionally substituted C₄-C₂₀ alkyl, optionally substituted C₂-C₁₄ alkenyl, and —(CH₂)ₚCH(OR⁶)(OR⁷);
R³ is selected from the group consisting of optionally substituted C₄-C₂₀ alkyl, optionally substituted C₂-C₁₄ alkenyl, or (CH₂) \CH(OR⁸)(OR⁹);
R^{1a} is:

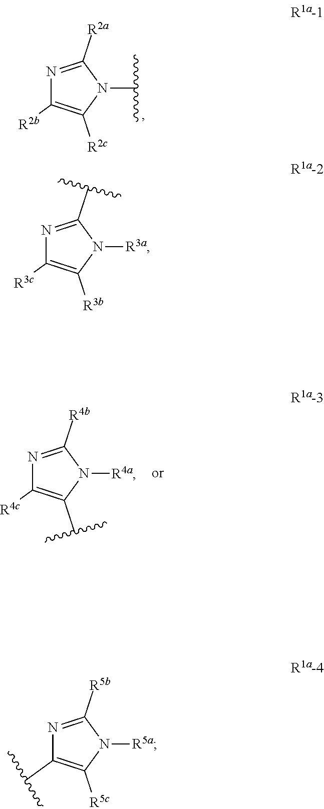

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl;
R^{5a}, R^{5b}, and R^{5c} are independently hydrogen and C₁-C₆ alkyl;
R⁶, R⁷, R⁸, and R⁹ are independently optionally substituted C₁-C₁₄ alkyl, optionally substituted C₂-C₁₄ alkenyl, or —(CH₂)ₘ-A-(CH₂)ₙH;
each A is independently a C₃-C₈ cycloalkylenyl;
each m is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
p is selected from the group consisting of 0, 1, 2, 3, 4, 5, 6, and 7; and
q is selected from the group consisting of 0, 1, 2, 3, 4, 5, 6, and 7.
Formulas (CY-I), (CY-II), (CY-III), (CY-IV), (CY-V), (CY-VI), (CY-VII), (CY-VIII), (CY-IX), (CY-IV-a), (CY-IV-b), (CY-IV-c), (CY-IV-d), (CY-IV-e), and (CY-IV-f)

In some embodiments, the present disclosure comprises a compound of any of the below Formulae:

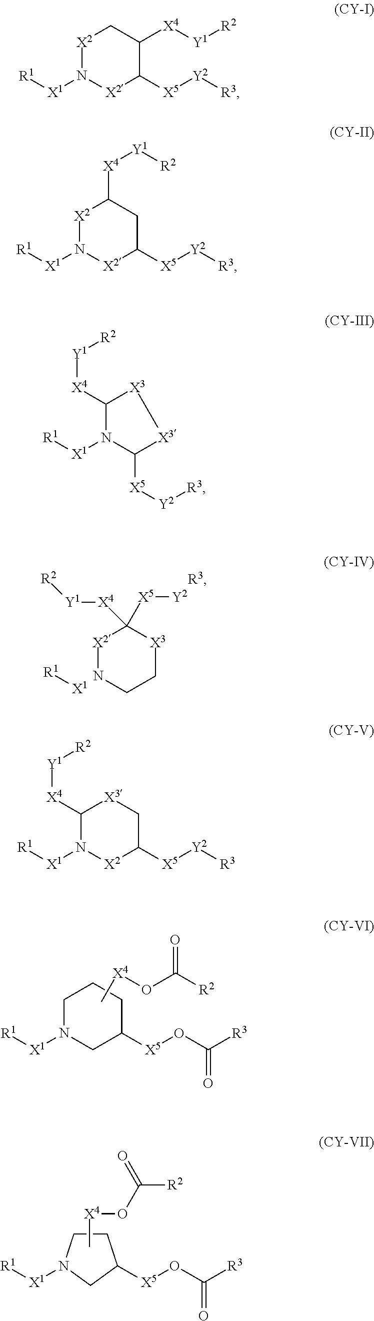

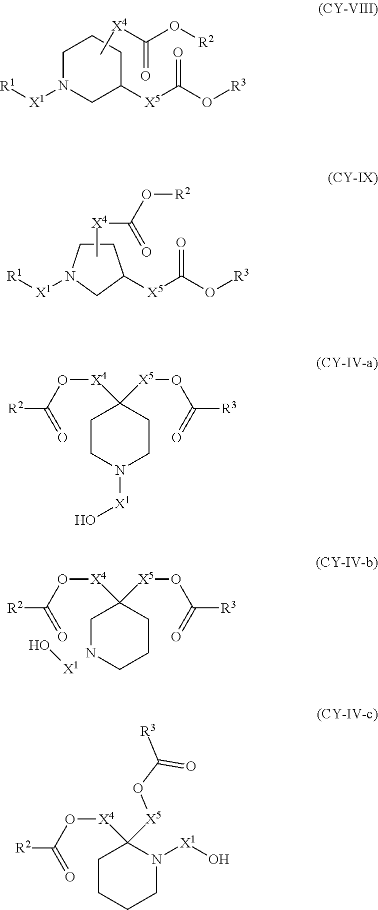

### Formula (CY-IV′)

In some embodiments, ionizable lipids of the present disclosure have a structure of Formula (CYIV′):

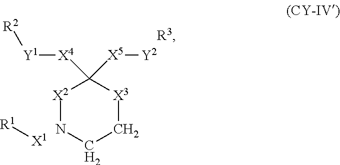

or a pharmaceutically acceptable salt thereof, wherein R¹, R², R³, X¹, X², X³, X⁴, X⁵, Y¹, and Y² are as defined in connection with Formula (CY-I′).

### Formula (CY-VI′)

In some embodiments, ionizable lipids of the present disclosure have a structure of Formula (CYVI′):

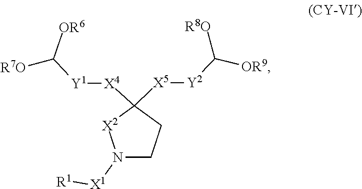

or a pharmaceutically acceptable salt thereof, wherein R¹, R⁶, R⁷, R⁸, R⁹, X¹, X², X³, X⁴, X⁵, Y¹, and Y² are as defined in connection with Formula (CY-I′).

In some embodiments, ionizable lipids of the present disclosure have a structure of Formula (CYVI′), or a pharmaceutically acceptable salt thereof, wherein R¹ is —OH.

In some embodiments, ionizable lipids of the present disclosure have a structure of Formula (CYVI′), or a pharmaceutically acceptable salt thereof, wherein X¹ is C₂-C₆ alkylenyl.

In some embodiments, ionizable lipids of the present disclosure have a structure of Formula (CYVI′), or a pharmaceutically acceptable salt thereof, wherein X² is —CH₂CH₂—.

In some embodiments, ionizable lipids of the present disclosure have a structure of Formula (CYVI′), or a pharmaceutically acceptable salt thereof, wherein X⁴ is C₂-C₆ alkylenyl.

In some embodiments, ionizable lipids of the present disclosure have a structure of Formula (CYVI′), or a pharmaceutically acceptable salt thereof, wherein X⁵ is C₂-C₆ alkylenyl.

In some embodiments, ionizable lipids of the present disclosure have a structure of Formula (CYVI′), or a pharmaceutically acceptable salt thereof, wherein Y¹ is:

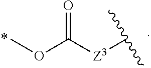

In some embodiments, ionizable lipids of the present disclosure have a structure of Formula (CYVI′), or a pharmaceutically acceptable salt thereof, wherein Y² is:

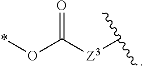

In some embodiments, ionizable lipids of the present disclosure have a structure of Formula (CYVI′), or a pharmaceutically acceptable salt thereof, wherein each Z³ is independently optionally substituted C₁-C₆ alkylenyl.

In some embodiments, ionizable lipids of the present disclosure have a structure of Formula (CYVI′), or a pharmaceutically acceptable salt thereof, wherein each Z³ is CH₂CH₂.

In some embodiments, ionizable lipids of the present disclosure have a structure of Formula (CYVI′), or a pharmaceutically acceptable salt thereof, wherein R⁶ is C₅-C₁₄ alkyl.

In some embodiments, ionizable lipids of the present disclosure have a structure of Formula (CYVI′), or a pharmaceutically acceptable salt thereof, wherein R⁷ is C₅-C₁₄ alkyl.

In some embodiments, ionizable lipids of the present disclosure have a structure of Formula (CYVI′), or a pharmaceutically acceptable salt thereof, wherein R⁶ is C₆-C₁₄ alkenyl.

In some embodiments, ionizable lipids of the present disclosure have a structure of Formula (CYVI′), or a pharmaceutically acceptable salt thereof, wherein R⁷ is C₆-C₁₄ alkenyl.

In some embodiments, ionizable lipids of the present disclosure have a structure of Formula (CYVI′), or a pharmaceutically acceptable salt thereof, wherein R⁸ is C₅-C₁₆ alkyl.

In some embodiments, ionizable lipids of the present disclosure have a structure of Formula (CYVI′), or a pharmaceutically acceptable salt thereof, wherein R⁹ is C₅-C₁₄ alkyl.

In some embodiments, ionizable lipids of the present disclosure have a structure of Formula (CYVI′), or a pharmaceutically acceptable salt thereof, wherein R⁸ is C₆-C₁₄ alkenyl.

In some embodiments, ionizable lipids of the present disclosure have a structure of Formula (CYVI′), or a pharmaceutically acceptable salt thereof, wherein R⁹ is C₆-C₁₄ alkenyl.

In some embodiments, ionizable lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

### R¹

In some embodiments, R¹ is selected from the group consisting of —OH, —OAc, R^{1a},

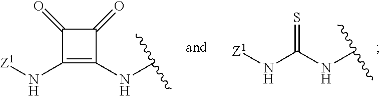

In some embodiments, R¹ is —OH or —OAc. In some embodiments, R¹ is OH. In some embodiments, R¹ is —OAc. In some embodiments, R¹ is R^{1a}. In some embodiments, R¹ is imidazolyl. In some embodiments, R¹ is

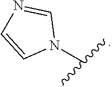 [SMILES: *n1ccnc1]

### R²

In some embodiments, R² is selected from the group consisting of optionally substituted C₄-C₂₀ alkyl, optionally substituted C₂-C₁₄ alkenyl, and —(CH₂)ₚCH(OR⁶)(OR⁷).

In some embodiments, R² is optionally substituted C₄-C₂₀ alkyl. In some embodiments, R² is optionally substituted C₈-C₁₇ alkyl. In some embodiments, R² is optionally substituted C₉-C₁₆ alkyl. In some embodiments, R² is optionally substituted C₈-C₁₀ alkyl. In some embodiments, R² is optionally substituted C₁₁-C₁₃ alkyl. In some embodiments, R² is optionally substituted C₁₄-C₁₆ alkyl. In some embodiments, R² is optionally substituted C₉ alkyl. In some embodiments, R² is optionally substituted C₁₀ alkyl. In some embodiments, R² is optionally substituted C₁₁ alkyl. In some embodiments, R² is optionally substituted C₁₂ alkyl. In some embodiments, R² is optionally substituted C₁₃ alkyl. In some embodiments, R² is optionally substituted C₁₄ alkyl. In some embodiments, R² is optionally substituted C₁₅ alkyl. In some embodiments, R² is optionally substituted C₁₆ alkyl.

In some embodiments, R² is optionally substituted C₂-C₁₄ alkenyl. In some embodiments, R² is optionally substituted C₅-C₁₄ alkenyl. In some embodiments, R² is optionally substituted C₇-C₁₄ alkenyl. In some embodiments, R² is optionally substituted C₉-C₁₄ alkenyl. In some embodiments, R² is optionally substituted C₁₀-C₁₄ alkenyl. In some embodiments, R² is optionally substituted C₁₂-C₁₄ alkenyl.

In some embodiments, R² is —(CH₂)ₚCH(OR⁶)(OR⁷). In some embodiments, R² is CH(OR⁶)(OR⁷). In some embodiments, R² is —CH₂CH(OR⁶)(OR⁷). In some embodiments, R² is —(CH₂)₂CH(OR⁶)(OR⁷). In some embodiments, R² is —(CH₂)₃CH(OR⁶)(OR⁷). In some embodiments, R² is —(CH₂)₄CH(OR⁶)(OR⁷).

In some embodiments, R² is selected from the group consisting of

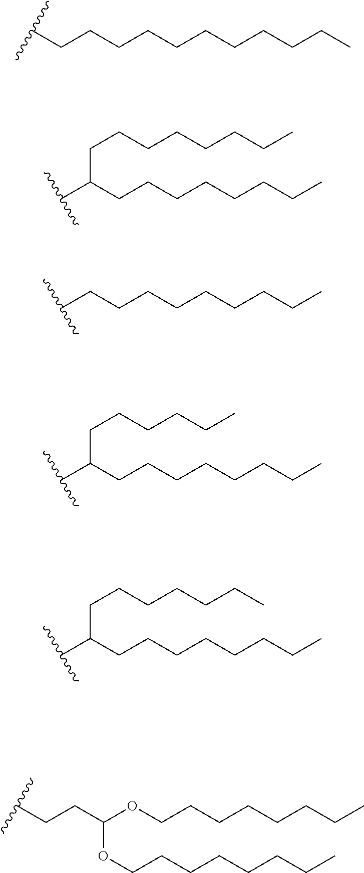

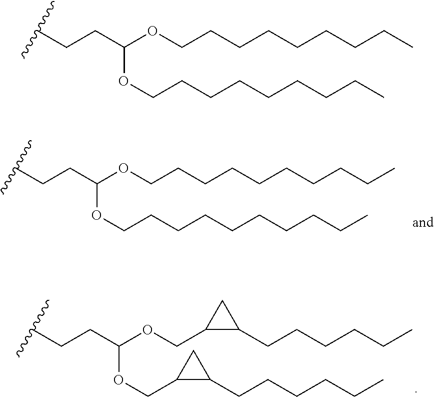 [SMILES: *CCC(OCCCCCCCCC)OCCCCCCCCC ; *CCC(OCCCCCCCCCC)OCCCCCCCCCC ; *CCC(OCC1CC1CCCCCC)OCC1CC1CCCCCC]

### R³

In some embodiments, R³ is selected from the group consisting of optionally substituted C₄-C₂₀ alkyl, optionally substituted C₂-C₁₄ alkenyl, and —(CH₂)_{q}CH(OR⁶)(OR⁷).

In some embodiments, R³ is optionally substituted C₄-C₂₀ alkyl. In some embodiments, R³ is optionally substituted C₈-C₁₇ alkyl. In some embodiments, R³ is optionally substituted C₉-C₁₆ alkyl. In some embodiments, R³ is optionally substituted C₈-C₁₀ alkyl. In some embodiments, R³ is optionally substituted C₁₁-C₁₃ alkyl. In some embodiments, R³ is optionally substituted C₁₄-C₁₆ alkyl. In some embodiments, R³ is optionally substituted C₉ alkyl. In some embodiments, R³ is optionally substituted C₁₀ alkyl. In some embodiments, R³ is optionally substituted C₁₁ alkyl. In some embodiments, R³ is optionally substituted C₁₂ alkyl. In some embodiments, R³ is optionally substituted C₁₃ alkyl. In some embodiments, R³ is optionally substituted C₁₄ alkyl. In some embodiments, R³ is optionally substituted C₁₅ alkyl. In some embodiments, R³ is optionally substituted C₁₆ alkyl.

In some embodiments, R³ is optionally substituted C₂-C₁₄ alkenyl. In some embodiments, R³ is optionally substituted C₅-C₁₄ alkenyl. In some embodiments, R³ is optionally substituted C₇-C₁₄ alkenyl. In some embodiments, R³ is optionally substituted C₉-C₁₄ alkenyl. In some embodiments, R³ is optionally substituted C₁₀-C₁₄ alkenyl. In some embodiments, R³ is optionally substituted C₁₂-C₁₄ alkenyl.

In some embodiments, R³ is (CH₂)_{q}CH(OR⁸)(OR⁹). In some embodiments, R³ is CH(OR⁸)(OR⁹). In some embodiments, R³ is CH₂CH(OR⁸)(OR⁹). In some embodiments, R³ is (CH₂)₂CH(OR⁸)(OR⁹). In some embodiments, R³ is (CH₂)₃CH(OR⁸)(OR⁹). In some embodiments, R³ is (CH₂)₄CH(OR⁸)(OR⁹).

In some embodiments, R³ is selected from the group consisting of

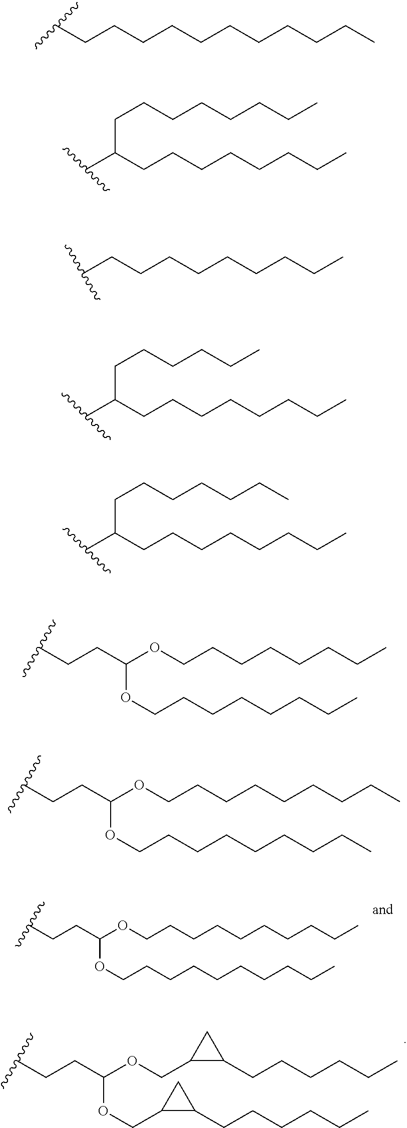

R⁶, R⁷, R⁸, R⁹

In some embodiments, R⁶, R⁷, R⁸, and R⁹ are independently optionally substituted C₁-C₁₄ alkyl, optionally substituted C₂-C₁₄ alkenyl, or —(CH₂)ₘ-A-(CH₂)ₙH. In some embodiments, R⁶, R⁷, R⁸, and R⁹ are independently optionally substituted C₁-C₁₄ alkyl. In some embodiments, R⁶, R⁷, R⁸, and R⁹ are independently optionally substituted C₂-C₁₄ alkenyl. In some embodiments, R⁶, R⁷, R⁸, and R⁹ are independently —(CH₂)ₘ-A-(CH₂)ₙH.

In some embodiments, R⁶ is optionally substituted C₁-C₁₄ alkyl, optionally substituted C₂-C₁₄ alkenyl, or —(CH₂)ₘ-A-(CH₂)ₙH. In some embodiments, R⁶ is optionally substituted C₃-C₁₀ alkyl. In some embodiments, R⁶ is optionally substituted C₄-C₁₀ alkyl. In some embodiments, R⁶ is independently optionally substituted C₅-C₁₀ alkyl. In some embodiments, R⁶ is optionally substituted C₉-C₁₀ alkyl. In some embodiments, R⁶ is optionally substituted C₁-C₁₄ alkyl. In some embodiments, R⁶ is optionally substituted C₂-C₁₄ alkenyl. In some embodiments, R⁶ is —(CH₂)ₘ-A-(CH₂)ₙH.

In some embodiments, R⁷ is optionally substituted C₁-C₁₄ alkyl, optionally substituted C₂-C₁₄ alkenyl, or —(CH₂)ₘ-A-(CH₂)ₙH. In some embodiments, R⁷ is optionally substituted C₃-C₁₀ alkyl. In some embodiments, R⁷ is optionally substituted C₄-C₁₀ alkyl. In some embodiments, R⁷ is optionally substituted C₅-C₁₀ alkyl. In some embodiments, R⁷ is optionally substituted C₉-C₁₀ alkyl. In some embodiments, R⁷ is optionally substituted C₁-C₁₄ alkyl. In some embodiments, R⁷ is optionally substituted optionally substituted C₂-C₁₄ alkenyl. In some embodiments, R⁷ is —(CH₂)ₘ-A-(CH₂)ₙH.

In some embodiments, R⁸ is optionally substituted C₁-C₁₄ alkyl, optionally substituted C₂-C₁₄ alkenyl, or —(CH₂)ₘ-A-(CH₂)ₙH. In some embodiments, R⁸ is optionally substituted C₃-C₁₀ alkyl. In some embodiments, R⁸ is optionally substituted C₄-C₁₀ alkyl. In some embodiments, R⁸ is optionally substituted C₅-C₁₀ alkyl. In some embodiments, R⁸ is optionally substituted C₉-C₁₀ alkyl. In some embodiments, R⁸ is optionally substituted C₁-C₁₄ alkyl. In some embodiments, R⁸ is optionally substituted C₂-C₁₄ alkenyl. In some embodiments, R⁸ is —(CH₂)ₘ-A-(CH₂)ₙH.

In some embodiments, R⁹ is optionally substituted C₁-C₁₄ alkyl, optionally substituted C₂-C₁₄ alkenyl, or —(CH₂)ₘ-A-(CH₂)ₙH. In some embodiments, R⁹ is optionally substituted C₃-C₁₀ alkyl. In some embodiments, R⁹ is optionally substituted C₄-C₁₀ alkyl. In some embodiments, R⁹ is optionally substituted C₅-C₁₀ alkyl. In some embodiments, R⁹ is optionally substituted C₉-C₁₀ alkyl. In some embodiments, R⁹ is optionally substituted C₁-C₁₄ alkyl. In some embodiments, R⁹ is optionally substituted C₂-C₁₄ alkenyl. In some embodiments, R⁹ is —(CH₂)ₘ-A-(CH₂)ₙH.

In some embodiments, each m is independently 0, 1, 2, 3, 4, 5, 6, 7, 8, 9, 10, 11, or 12. In some embodiments, each m is 0. In some embodiments, each m is 1. In some embodiments, each m is 2. In some embodiments, each m is 3. In some embodiments, each m is 4. In some embodiments, each m is 5. In some embodiments, each m is 6. In some embodiments, each m is 7. In some embodiments, each m is 8. In some embodiments, each m is 9. In some embodiments, each m is 10. In some embodiments, each m is 11. In some embodiments, each m is 12.

In some embodiments, each n is independently 0, 1, 2, 3, 4, 5, 6, 7, 8, 9, 10, 11, or 12. In some embodiments, each n is 0. In some embodiments, each n is 1. In some embodiments, each n is 2. In some embodiments, each n is 3. In some embodiments, each n is 4. In some embodiments, each n is 5. In some embodiments, each n is 6. In some embodiments, each n is 7. In some embodiments, each n is 8. In some embodiments, each n is 9. In some embodiments, each n is 10. In some embodiments, each n is 11. In some embodiments, each n is 12.

In some embodiments, each A is independently a C₃-C₈ cycloalkylenyl. In some embodiments, each A is cyclopropylenyl.

### X¹

In some embodiments, X¹ is optionally substituted C₂-C₆ alkylenyl. In some embodiments, X¹ is optionally substituted C₂-C₅ alkylenyl. In some embodiments, X¹ is optionally substituted C₂-C₄ alkylenyl. In some embodiments, X¹ is optionally substituted C₂-C₃ alkylenyl. In some embodiments, X¹ is optionally substituted C₂ alkylenyl. In some embodiments, X¹ is optionally substituted C₃ alkylenyl. In some embodiments, X¹ is optionally substituted C₄ alkylenyl. In some embodiments, X¹ is optionally substituted C₅ alkylenyl. In some embodiments, X¹ is optionally substituted C₆ alkylenyl. In some embodiments, X¹ is optionally substituted —(CH₂)₂—. In some embodiments, X¹ is optionally substituted —(CH₂)₃—. In some embodiments, X¹ is optionally substituted —(CH₂)₄—. In some embodiments, X¹ is optionally substituted —(CH₂)₅—. In some embodiments, X¹ is optionally substituted —(CH₂)₆—.

### X²

In some embodiments, X² is selected from the group consisting of a bond, —CH₂-and-CH₂CH₂—. In some embodiments, X² is a bond. In some embodiments, X² is —CH₂—. In some embodiments, X² is —CH₂CH₂—.

### X^{2′}

In some embodiments, X^{2′} is selected from the group consisting of a bond, —CH₂-and-CH₂CH₂—. In some embodiments, X^{2′} is a bond. In some embodiments, X^{2′} is —CH₂—. In some embodiments, X^{2′} is —CH₂CH₂—.

### X³

In some embodiments, X³ is selected from the group consisting of a bond, —CH₂-and-CH₂CH₂—. In some embodiments, X³ is a bond. In some embodiments, X³ is —CH₂—. In some embodiments, X³ is —CH₂CH₂—.

### X^{3′}

In some embodiments, X^{3′} is selected from the group consisting of a bond, —CH₂— and —CH₂CH₂—. In some embodiments, X^{3′} is a bond. In some embodiments, X^{3′} is —CH₂—. In some embodiments, X^{3′} is —CH₂CH₂—.

### X⁴

In some embodiments, X⁴ is selected from the group consisting of optionally substituted C₂-C₁₄ alkylenyl and optionally substituted C₂-C₁₄ alkenylenyl. In some embodiments, X⁴ is optionally substituted C₂-C₁₄ alkylenyl. In some embodiments, X⁴ is optionally substituted C₂-C₁₀ alkylenyl. In some embodiments, X⁴ is optionally substituted C₂-C₈ alkylenyl. In some embodiments, X⁴ is optionally substituted C₂-C₆ alkylenyl. In some embodiments, X⁴ is optionally substituted C₃-C₆ alkylenyl. In some embodiments, X⁴ is optionally substituted C₃ alkylenyl. In some embodiments, X⁴ is optionally substituted C₄ alkylenyl. In some embodiments, X⁴ is optionally substituted C₅ alkylenyl. In some embodiments, X⁴ is optionally substituted C₆ alkylenyl. In some embodiments, X⁴ is optionally substituted —(CH₂)₂—. In some embodiments, X⁴ is optionally substituted —(CH₂)₃—. In some embodiments, X⁴ is optionally substituted —(CH₂)₄—. In some embodiments, X⁴ is optionally substituted —(CH₂)₅—. In some embodiments, X⁴ is optionally substituted —(CH₂)₆—.

### X⁵

In some embodiments, X⁵ is selected from the group consisting of optionally substituted C₂-C₁₄ alkylenyl and optionally substituted C₂-C₁₄ alkenylenyl. In some embodiments, X⁵ is optionally substituted C₂-C₁₄ alkylenyl. In some embodiments, X⁵ is optionally substituted C₂-C₁₀ alkylenyl. In some embodiments, X⁵ is optionally substituted C₂-C₈ alkylenyl. In some embodiments, X⁵ is optionally substituted C₂-C₆ alkylenyl. In some embodiments, X⁵ is optionally substituted C₃-C₆ alkylenyl. In some embodiments, X⁵ is optionally substituted C₃ alkylenyl. In some embodiments, X⁵ is optionally substituted C₄ alkylenyl. In some embodiments, X⁵ is optionally substituted C₅ alkylenyl. In some embodiments, X⁵ is optionally substituted C₆ alkylenyl. In some embodiments, X⁵ is optionally substituted —(CH₂)₂—. In some embodiments, X⁵ is optionally substituted —(CH₂)₃—. In some embodiments, X⁵ is optionally substituted —(CH₂)₄—. In some embodiments, X⁵ is optionally substituted —(CH₂)₅—. In some embodiments, X⁵ is optionally substituted —(CH₂)₆—.

### Y¹

In some embodiments, Y¹ is selected from the group consisting of

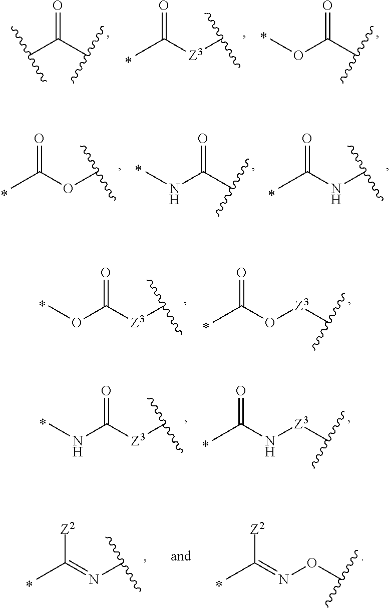

In some embodiments, Y¹ is

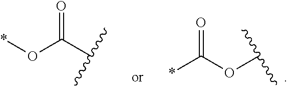 [SMILES: *OC(*)=O ; *OC(*)=O]

### Y²

In some embodiments, Y² is selected from the group consisting of

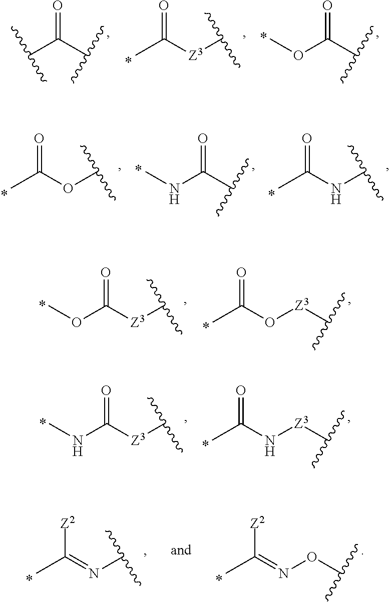

In some embodiments, Y² is

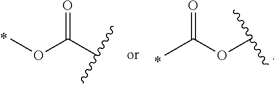

In some embodiments, Lipids of the Present Disclosure are selected from any lipid in Table (II) below or a pharmaceutically acceptable salt thereof:

TABLE II
Non-Limiting Examples of Ionizable Lipids with a Cyclic Core

| Structure | # |
|---|---|
| 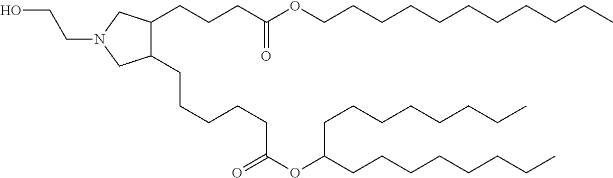 [SMILES: CCCCCCCCCCCOC(=O)CCCC1CN(CCO)CC1CCCCCC(=O)OC(CCCCCCCC)CCCCCCCC] | CY1 |
| 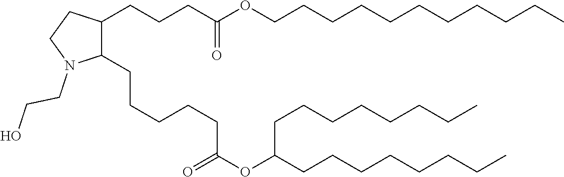 [SMILES: CCCCCCCCCCCOC(=O)CCCC1CCN(CCO)C1CCCCCC(=O)OC(CCCCCCCC)CCCCCCCC] | CY2 |
| 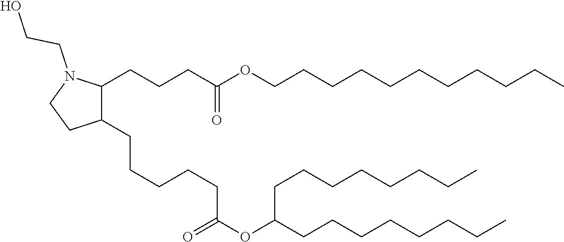 [SMILES: CCCCCCCCCCCOC(=O)CCCC1C(CCCCCC(=O)OC(CCCCCCCC)CCCCCCCC)CCN1CCO] | CY3 |
| 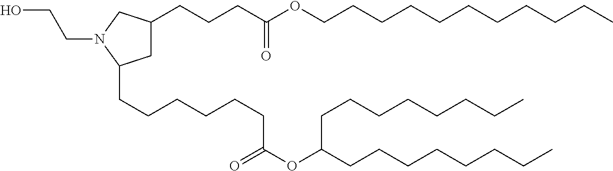 [SMILES: CCCCCCCCCCCOC(=O)CCCC1CC(CCCCCCC(=O)OC(CCCCCCCC)CCCCCCCC)N(CCO)C1] | CY4 |
| 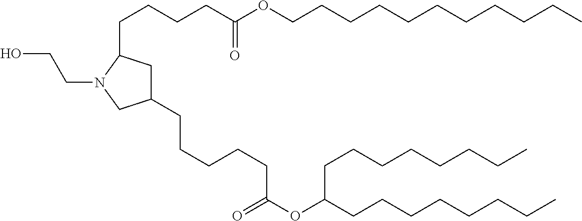 [SMILES: CCCCCCCCCCCOC(=O)CCCCC1CC(CCCCCC(=O)OC(CCCCCCCC)CCCCCCCC)CN1CCO] | CY5 |
| 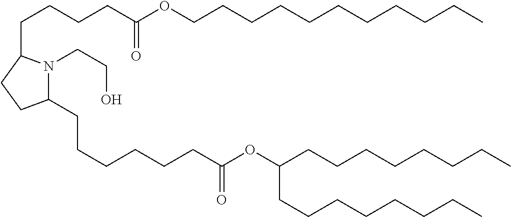 [SMILES: CCCCCCCCCCCOC(=O)CCCCC1CCC(CCCCCCC(=O)OC(CCCCCCCC)CCCCCCCC)N1CCO] | CY6 |
| 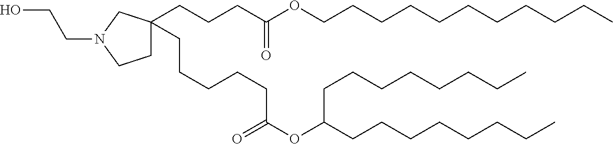 [SMILES: CCCCCCCCCCCOC(=O)CCCC1(CCCCCC(=O)OC(CCCCCCCC)CCCCCCCC)CCN(CCO)C1] | CY7 |
| 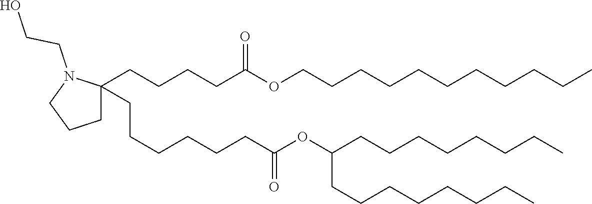 [SMILES: CCCCCCCCCCCOC(=O)CCCCC1(CCCCCCC(=O)OC(CCCCCCCC)CCCCCCCC)CCCN1CCO] | CY8 |
| 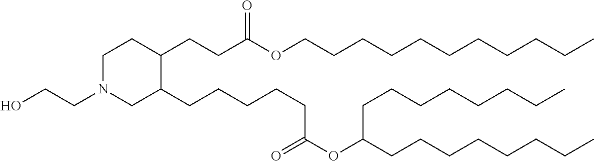 [SMILES: CCCCCCCCCCCOC(=O)CCC1CCN(CCO)CC1CCCCCC(=O)OC(CCCCCCCC)CCCCCCCC] | CY9 |
| 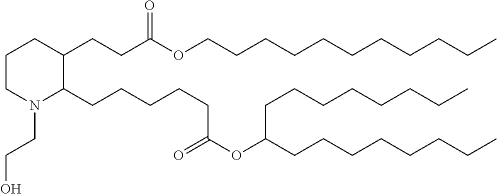 [SMILES: CCCCCCCCCCCOC(=O)CCC1CCCN(CCO)C1CCCCCC(=O)OC(CCCCCCCC)CCCCCCCC] | CY10 |
| 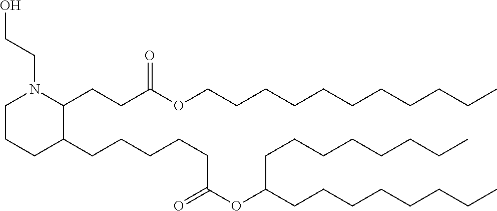 [SMILES: CCCCCCCCCCCOC(=O)CCC1C(CCCCCC(=O)OC(CCCCCCCC)CCCCCCCC)CCCN1CCO] | CY11 |
| 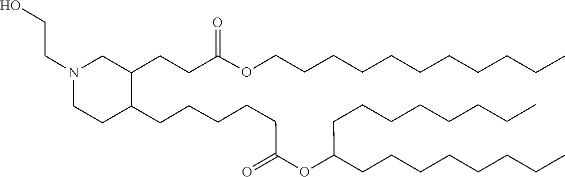 [SMILES: CCCCCCCCCCCOC(=O)CCC1CN(CCO)CCC1CCCCCC(=O)OC(CCCCCCCC)CCCCCCCC] | CY12 |
| 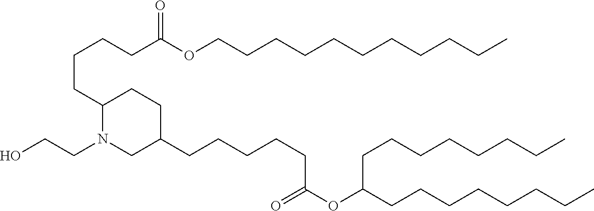 [SMILES: CCCCCCCCCCCOC(=O)CCCCC1CCC(CCCCCC(=O)OC(CCCCCCCC)CCCCCCCC)CN1CCO] | CY13 |
| 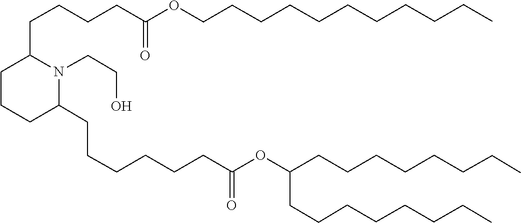 [SMILES: CCCCCCCCCCCOC(=O)CCCCC1CCCC(CCCCCCC(=O)OC(CCCCCCCC)CCCCCCCC)N1CCO] | CY14 |
| 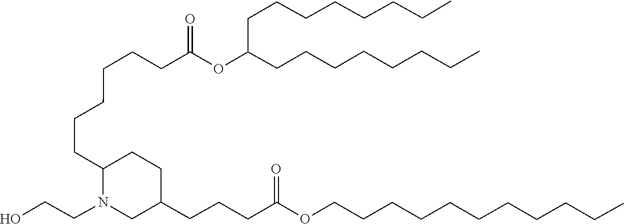 [SMILES: CCCCCCCCCCCOC(=O)CCCC1CCC(CCCCCCC(=O)OC(CCCCCCCC)CCCCCCCC)N(CCO)C1] | CY15 |
| 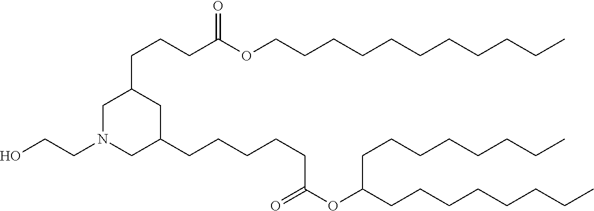 [SMILES: CCCCCCCCCCCOC(=O)CCCC1CC(CCCCCC(=O)OC(CCCCCCCC)CCCCCCCC)CN(CCO)C1] | CY16 |
| 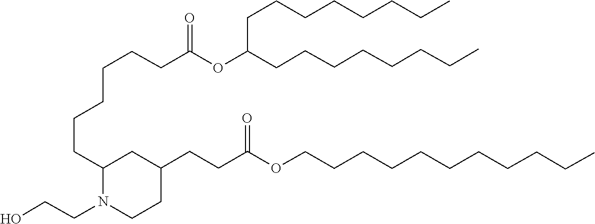 [SMILES: CCCCCCCCCCCOC(=O)CCC1CCN(CCO)C(CCCCCCC(=O)OC(CCCCCCCC)CCCCCCCC)C1] | CY17 |
| 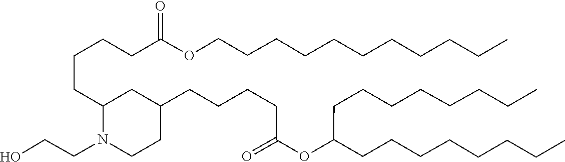 [SMILES: CCCCCCCCCCCOC(=O)CCCCC1CC(CCCCC(=O)OC(CCCCCCCC)CCCCCCCC)CCN1CCO] | CY18 |
| 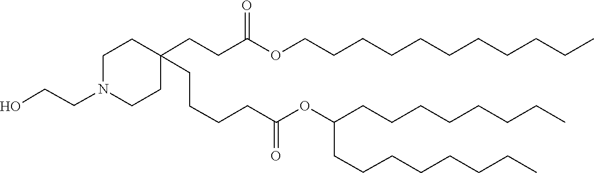 [SMILES: CCCCCCCCCCCOC(=O)CCC1(CCCCC(=O)OC(CCCCCCCC)CCCCCCCC)CCN(CCO)CC1] | CY19 |
| 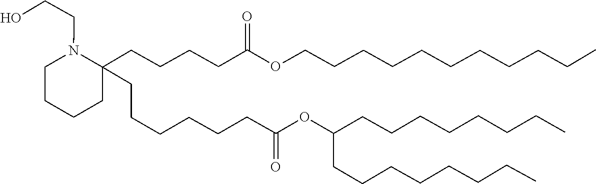 [SMILES: CCCCCCCCCCCOC(=O)CCCCC1(CCCCCCC(=O)OC(CCCCCCCC)CCCCCCCC)CCCCN1CCO] | CY20 |
| 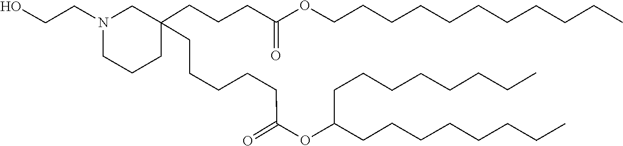 [SMILES: CCCCCCCCCCCOC(=O)CCCC1(CCCCCC(=O)OC(CCCCCCCC)CCCCCCCC)CCCN(CCO)C1] | CY21 |
| 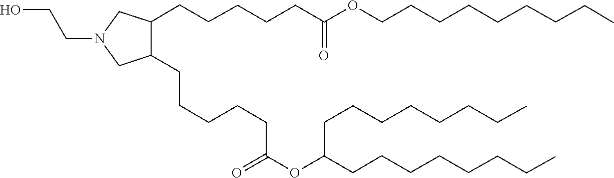 [SMILES: CCCCCCCCCOC(=O)CCCCCC1CN(CCO)CC1CCCCCC(=O)OC(CCCCCCCC)CCCCCCCC] | CY22 |
| 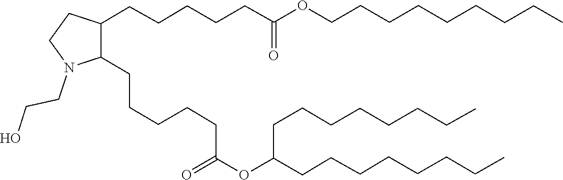 [SMILES: CCCCCCCCCOC(=O)CCCCCC1CCN(CCO)C1CCCCCC(=O)OC(CCCCCCCC)CCCCCCCC] | CY23 |
| 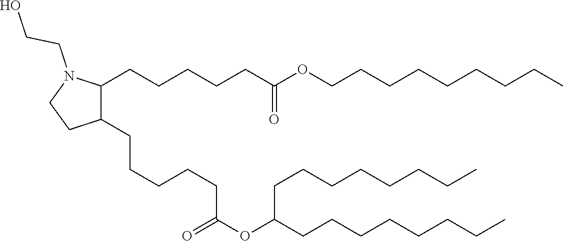 [SMILES: CCCCCCCCCOC(=O)CCCCCC1C(CCCCCC(=O)OC(CCCCCCCC)CCCCCCCC)CCN1CCO] | CY24 |
| 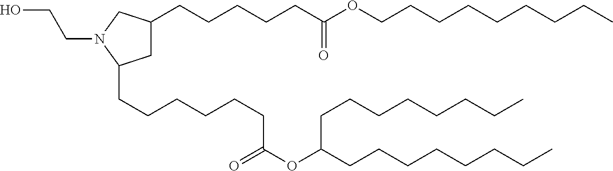 [SMILES: CCCCCCCCCOC(=O)CCCCCC1CC(CCCCCCC(=O)OC(CCCCCCCC)CCCCCCCC)N(CCO)C1] | CY25 |
| 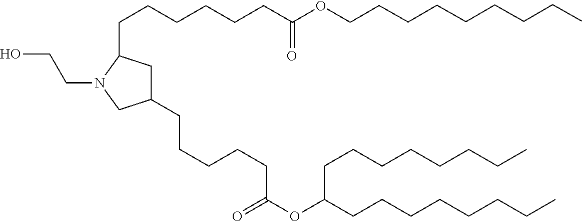 [SMILES: CCCCCCCCCOC(=O)CCCCCCC1CC(CCCCCC(=O)OC(CCCCCCCC)CCCCCCCC)CN1CCO] | CY26 |
| 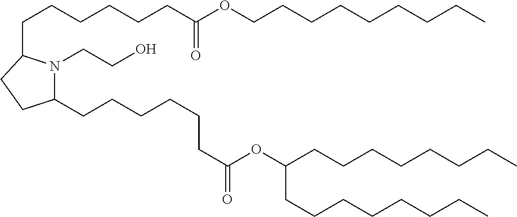 [SMILES: CCCCCCCCCOC(=O)CCCCCCC1CCC(CCCCCCC(=O)OC(CCCCCCCC)CCCCCCCC)N1CCO] | CY27 |
| 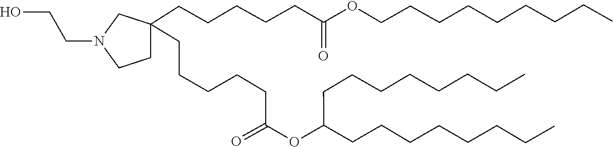 [SMILES: CCCCCCCCCOC(=O)CCCCCC1(CCCCCC(=O)OC(CCCCCCCC)CCCCCCCC)CCN(CCO)C1] | CY28 |
| 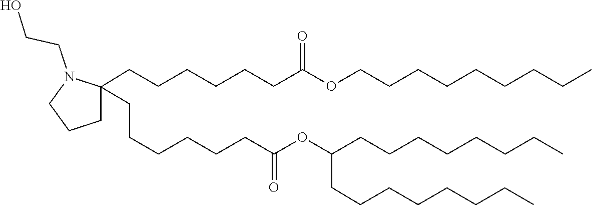 [SMILES: CCCCCCCCCOC(=O)CCCCCCC1(CCCCCCC(=O)OC(CCCCCCCC)CCCCCCCC)CCCN1CCO] | CY29 |
| 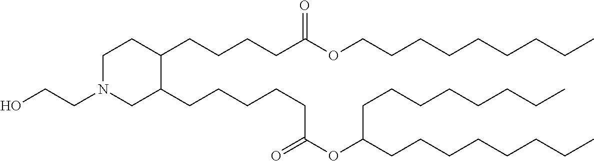 [SMILES: CCCCCCCCCOC(=O)CCCCC1CCN(CCO)CC1CCCCCC(=O)OC(CCCCCCCC)CCCCCCCC] | CY30 |
| 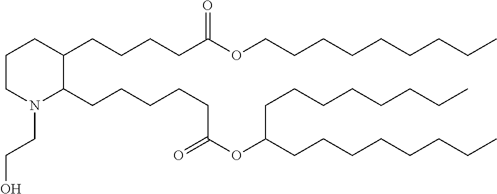 [SMILES: CCCCCCCCCOC(=O)CCCCC1CCCN(CCO)C1CCCCCC(=O)OC(CCCCCCCC)CCCCCCCC] | CY31 |
| 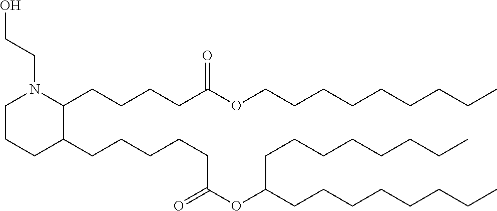 [SMILES: CCCCCCCCCOC(=O)CCCCC1C(CCCCCC(=O)OC(CCCCCCCC)CCCCCCCC)CCCN1CCO] | CY32 |
| 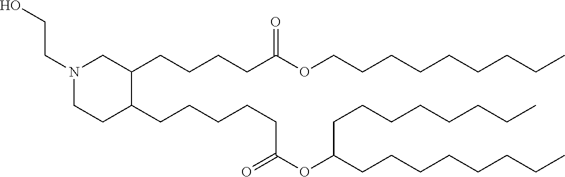 [SMILES: CCCCCCCCCOC(=O)CCCCC1CN(CCO)CCC1CCCCCC(=O)OC(CCCCCCCC)CCCCCCCC] | CY33 |
| 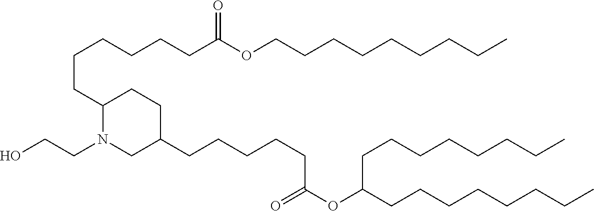 [SMILES: CCCCCCCCCOC(=O)CCCCCCC1CCC(CCCCCC(=O)OC(CCCCCCCC)CCCCCCCC)CN1CCO] | CY34 |
| 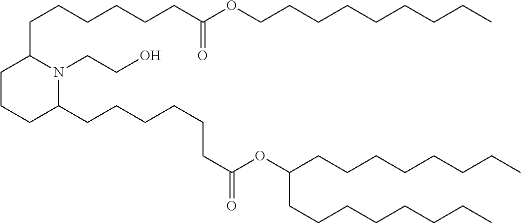 [SMILES: CCCCCCCCCOC(=O)CCCCCCC1CCCC(CCCCCCC(=O)OC(CCCCCCCC)CCCCCCCC)N1CCO] | CY35 |
| 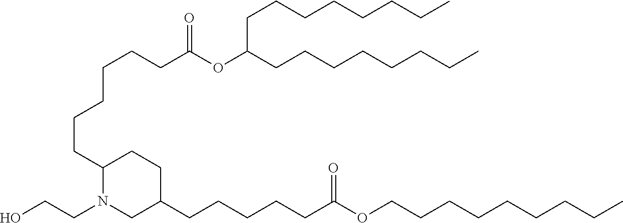 [SMILES: CCCCCCCCCOC(=O)CCCCCC1CCC(CCCCCCC(=O)OC(CCCCCCCC)CCCCCCCC)N(CCO)C1] | CY36 |
| 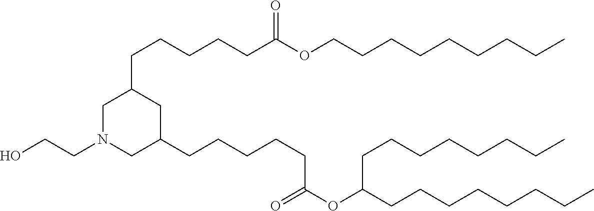 [SMILES: CCCCCCCCCOC(=O)CCCCCC1CC(CCCCCC(=O)OC(CCCCCCCC)CCCCCCCC)CN(CCO)C1] | CY37 |
| 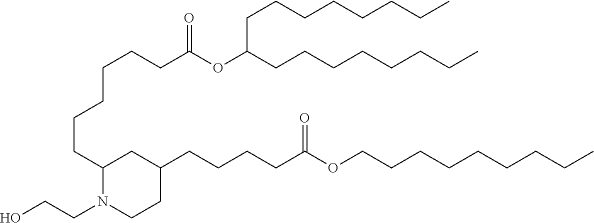 [SMILES: CCCCCCCCCOC(=O)CCCCC1CCN(CCO)C(CCCCCCC(=O)OC(CCCCCCCC)CCCCCCCC)C1] | CY38 |
| 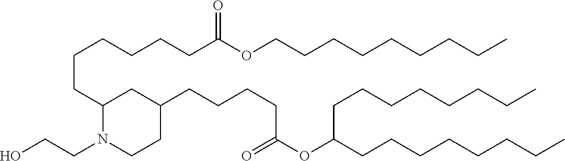 [SMILES: CCCCCCCCCOC(=O)CCCCCCC1CC(CCCCC(=O)OC(CCCCCCCC)CCCCCCCC)CCN1CCO] | CY39 |
| 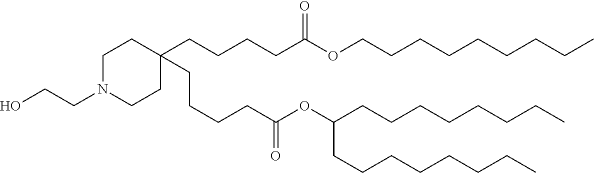 [SMILES: CCCCCCCCCOC(=O)CCCCC1(CCCCC(=O)OC(CCCCCCCC)CCCCCCCC)CCN(CCO)CC1] | CY40 |
| 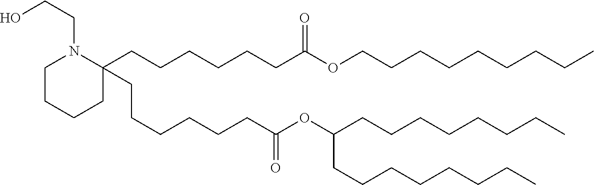 [SMILES: CCCCCCCCCOC(=O)CCCCCCC1(CCCCCCC(=O)OC(CCCCCCCC)CCCCCCCC)CCCCN1CCO] | CY41 |
| 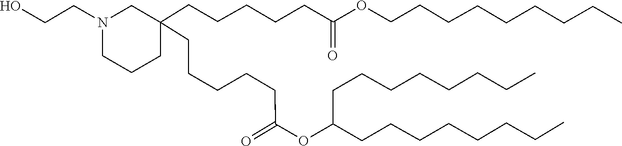 [SMILES: CCCCCCCCCOC(=O)CCCCCC1(CCCCCC(=O)OC(CCCCCCCC)CCCCCCCC)CCCN(CCO)C1] | CY42 |
| 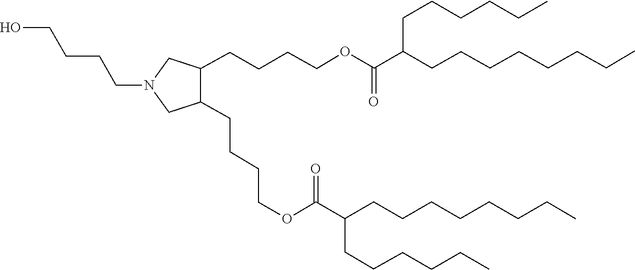 [SMILES: CCCCCCCCC(CCCCCC)C(=O)OCCCCC1CN(CCCCO)CC1CCCCOC(=O)C(CCCCCC)CCCCCCCC] | CY43 |
| 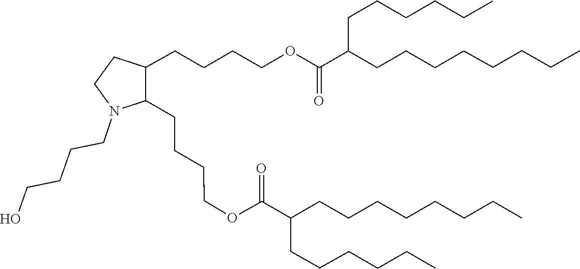 [SMILES: CCCCCCCCC(CCCCCC)C(=O)OCCCCC1CCN(CCCCO)C1CCCCOC(=O)C(CCCCCC)CCCCCCCC] | CY44 |
| 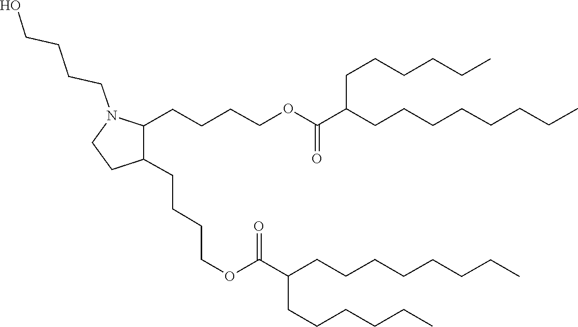 [SMILES: CCCCCCCCC(CCCCCC)C(=O)OCCCCC1CCN(CCCCO)C1CCCCOC(=O)C(CCCCCC)CCCCCCCC] | CY45 |
| 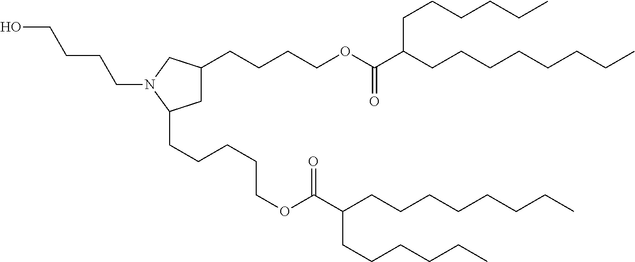 [SMILES: CCCCCCCCC(CCCCCC)C(=O)OCCCCCC1CC(CCCCOC(=O)C(CCCCCC)CCCCCCCC)CN1CCCCO] | CY46 |
| 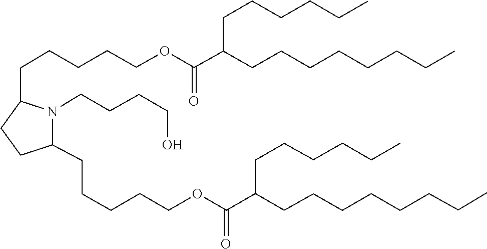 [SMILES: CCCCCCCCC(CCCCCC)C(=O)OCCCCCC1CCC(CCCCCOC(=O)C(CCCCCC)CCCCCCCC)N1CCCCO] | CY47 |
| 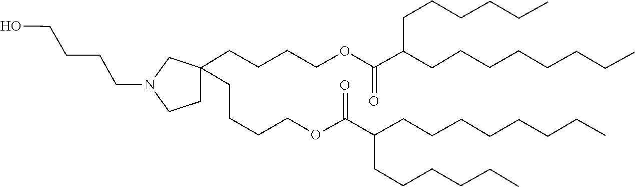 [SMILES: CCCCCCCCC(CCCCCC)C(=O)OCCCCC1(CCCCOC(=O)C(CCCCCC)CCCCCCCC)CCN(CCCCO)C1] | CY48 |
| 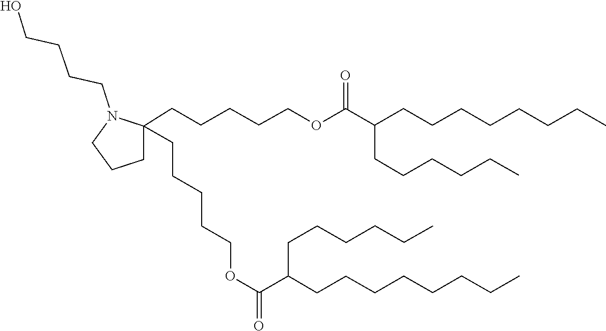 [SMILES: CCCCCCCCC(CCCCCC)C(=O)OCCCCCC1(CCCCCOC(=O)C(CCCCCC)CCCCCCCC)CCCN1CCCCO] | CY49 |
| 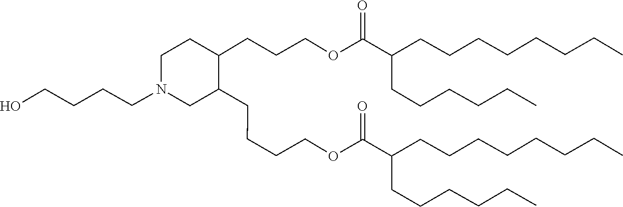 [SMILES: CCCCCCCCC(CCCCCC)C(=O)OCCCCC1CN(CCCCO)CCC1CCCOC(=O)C(CCCCCC)CCCCCCCC] | CY50 |
| 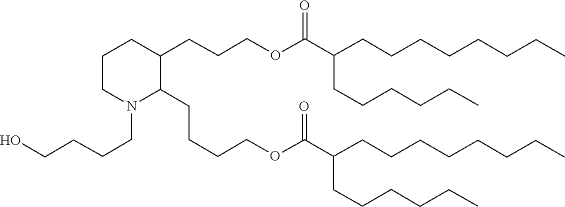 [SMILES: CCCCCCCCC(CCCCCC)C(=O)OCCCCC1C(CCCOC(=O)C(CCCCCC)CCCCCCCC)CCCN1CCCCO] | CY51 |
| 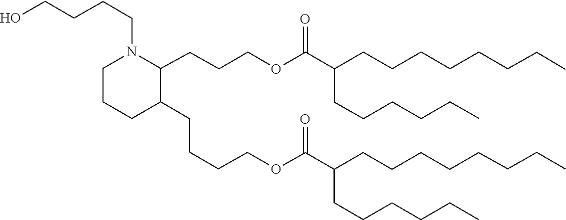 [SMILES: CCCCCCCCC(CCCCCC)C(=O)OCCCCC1CCCN(CCCCO)C1CCCOC(=O)C(CCCCCC)CCCCCCCC] | CY52 |
| 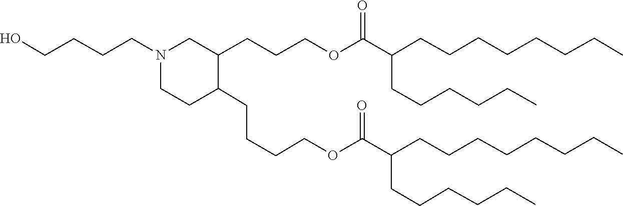 [SMILES: CCCCCCCCC(CCCCCC)C(=O)OCCCCC1CCN(CCCCO)CC1CCCOC(=O)C(CCCCCC)CCCCCCCC] | CY53 |
| 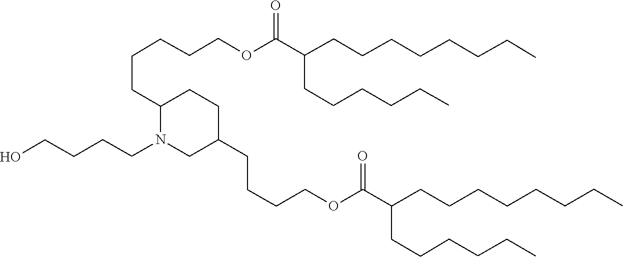 [SMILES: CCCCCCCCC(CCCCCC)C(=O)OCCCCCC1CCC(CCCCOC(=O)C(CCCCCC)CCCCCCCC)CN1CCCCO] | CY54 |
| 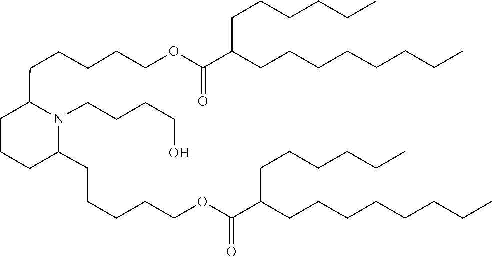 [SMILES: CCCCCCCCC(CCCCCC)C(=O)OCCCCCC1CCCC(CCCCCOC(=O)C(CCCCCC)CCCCCCCC)N1CCCCO] | CY55 |
| 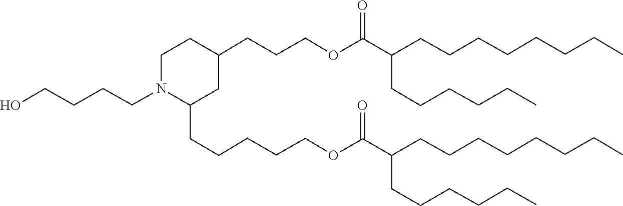 [SMILES: CCCCCCCCC(CCCCCC)C(=O)OCCCCCC1CC(CCCOC(=O)C(CCCCCC)CCCCCCCC)CCN1CCCCO] | CY56 |
| 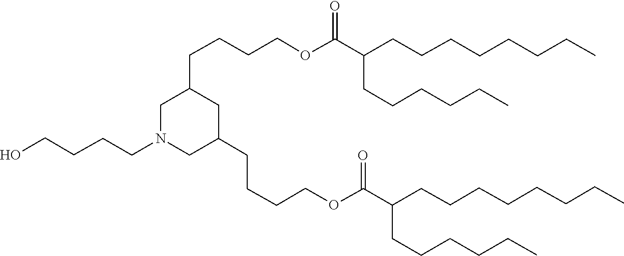 [SMILES: CCCCCCCCC(CCCCCC)C(=O)OCCCCC1CC(CCCCOC(=O)C(CCCCCC)CCCCCCCC)CN(CCCCO)C1] | CY57 |
| 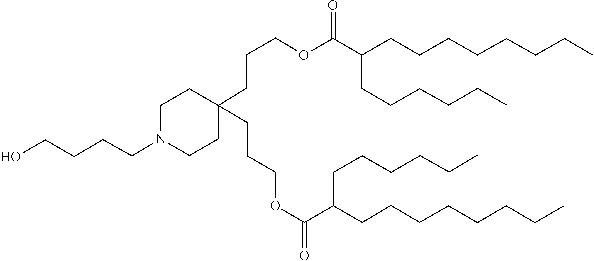 [SMILES: CCCCCCCCC(CCCCCC)C(=O)OCCCC1(CCCOC(=O)C(CCCCCC)CCCCCCCC)CCN(CCCCO)CC1] | CY58 |
| 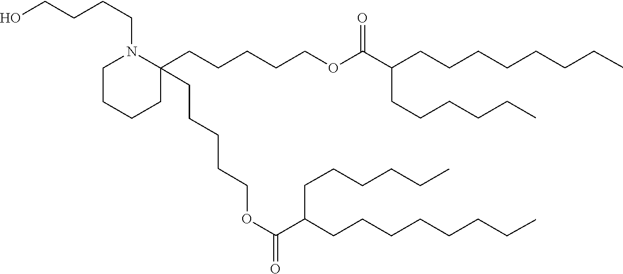 [SMILES: CCCCCCCCC(CCCCCC)C(=O)OCCCCCC1(CCCCCOC(=O)C(CCCCCC)CCCCCCCC)CCCCN1CCCCO] | CY59 |
| 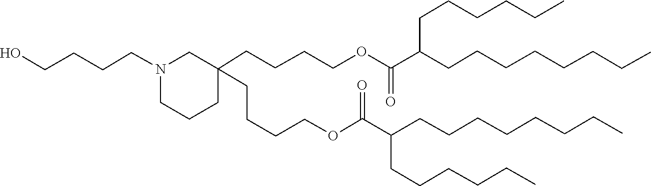 [SMILES: CCCCCCCCC(CCCCCC)C(=O)OCCCCC1(CCCCOC(=O)C(CCCCCC)CCCCCCCC)CCCN(CCCCO)C1] | CY60 |
| 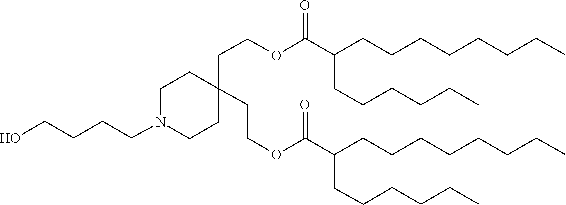 [SMILES: CCCCCCCCC(CCCCCC)C(=O)OCCC1(CCOC(=O)C(CCCCCC)CCCCCCCC)CCN(CCCCO)CC1] | CY61 |
| 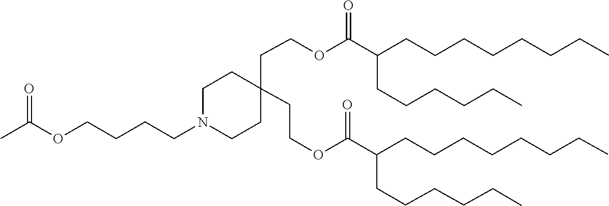 [SMILES: CCCCCCCCC(CCCCCC)C(=O)OCCC1(CCOC(=O)C(CCCCCC)CCCCCCCC)CCN(CCCCOC(C)=O)CC1] | CY62 |
| 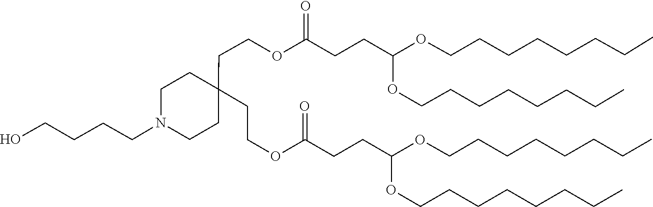 [SMILES: CCCCCCCCOC(CCC(=O)OCCC1(CCOC(=O)CCC(OCCCCCCCC)OCCCCCCCC)CCN(CCCCO)CC1)OCCCCCCCC] | CY63 |
| 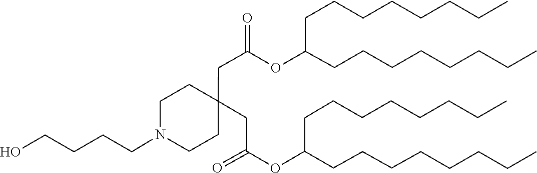 [SMILES: CCCCCCCCC(CCCCCCCC)OC(=O)CC1(CC(=O)OC(CCCCCCCC)CCCCCCCC)CCN(CCCCO)CC1] | CY64 |
| 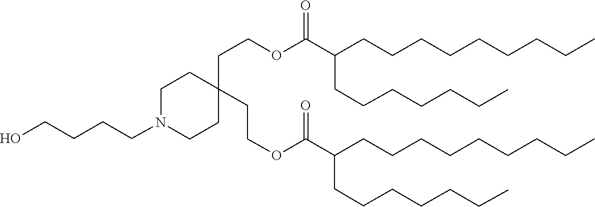 [SMILES: CCCCCCCCCC(CCCCCCC)C(=O)OCCC1(CCOC(=O)C(CCCCCCC)CCCCCCCCC)CCN(CCCCO)CC1] | CY65 |
| 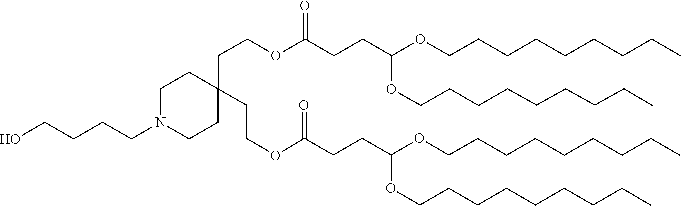 [SMILES: CCCCCCCCCOC(CCC(=O)OCCC1(CCOC(=O)CCC(OCCCCCCCCC)OCCCCCCCCC)CCN(CCCCO)CC1)OCCCCCCCCC] | CY66 |
| 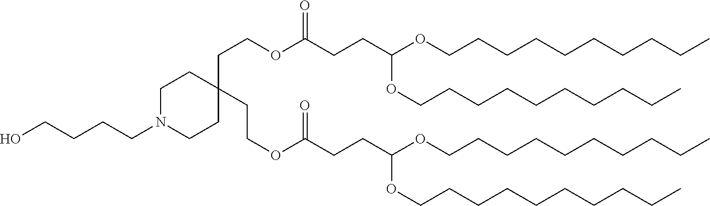 [SMILES: CCCCCCCCCCOC(CCC(=O)OCCC1(CCOC(=O)CCC(OCCCCCCCCCC)OCCCCCCCCCC)CCN(CCCCO)CC1)OCCCCCCCCCC] | CY67 |
| 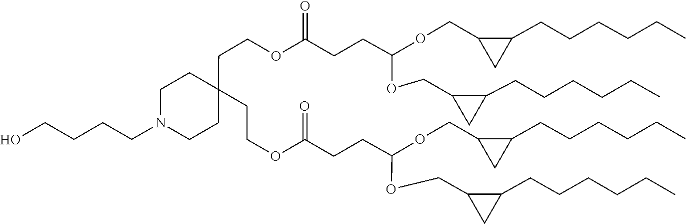 [SMILES: CCCCCCC1CC1COC(CCC(=O)OCCC1(CCOC(=O)CCC(OCC2CC2CCCCCC)OCC2CC2CCCCCC)CCN(CCCCO)CC1)OCC1CC1CCCCCC] | CY68 |
| 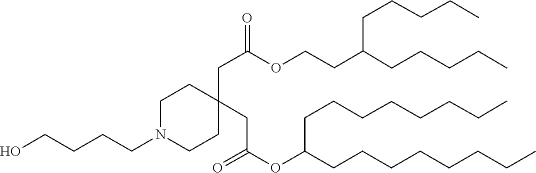 [SMILES: CCCCCCCCC(CCCCCCCC)OC(=O)CC1(CC(=O)OCCC(CCCCC)CCCCC)CCN(CCCCO)CC1] | CY69 |
| 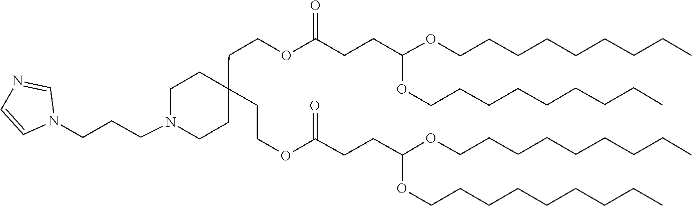 [SMILES: CCCCCCCCCOC(CCC(=O)OCCC1(CCOC(=O)CCC(OCCCCCCCCC)OCCCCCCCCC)CCN(CCCn2ccnc2)CC1)OCCCCCCCCC] | CY70 |
| 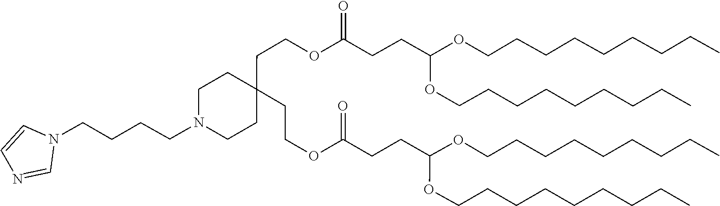 [SMILES: CCCCCCCCCOC(CCC(=O)OCCC1(CCOC(=O)CCC(OCCCCCCCCC)OCCCCCCCCC)CCN(CCCCn2ccnc2)CC1)OCCCCCCCCC] | CY71 |

### Series “C”

In some embodiments, an LNP of the present disclosure comprises an ionizable lipid disclosed in PCT Publication WO2023122752A1, which is incorporated by reference herein, in its entirety.

In one embodiment, the disclosure provides a compound of Formula IA:

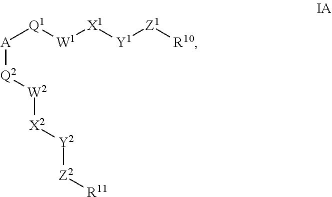

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

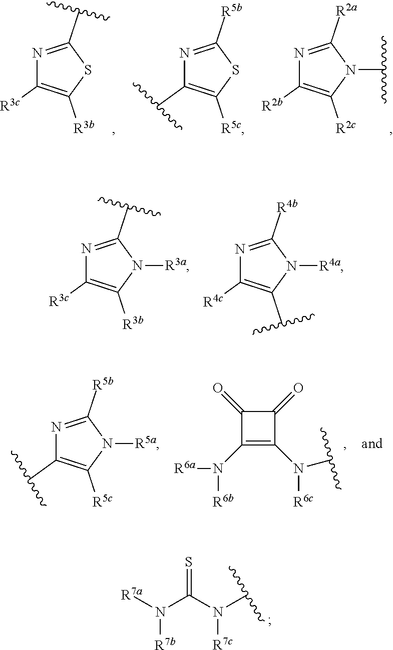

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

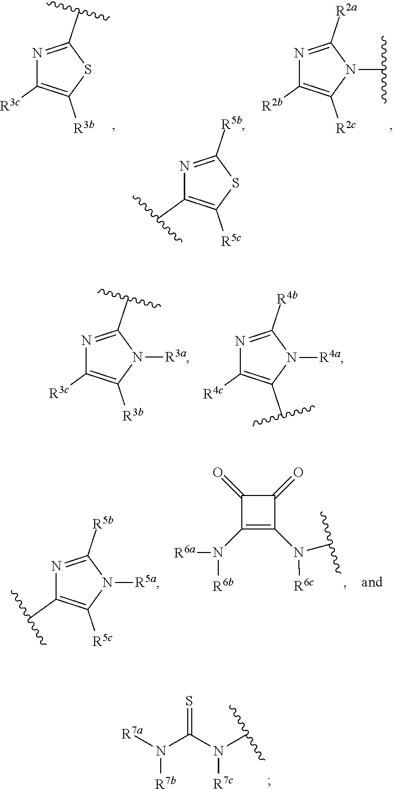

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R¹²²)C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₄-C₁₂ cycloalkylenyl,

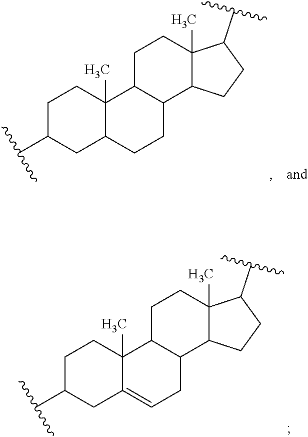 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

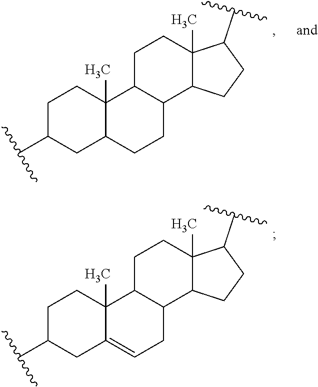 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IB:

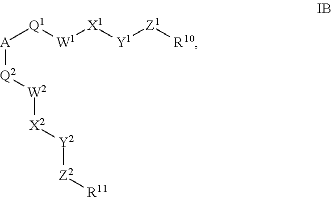

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

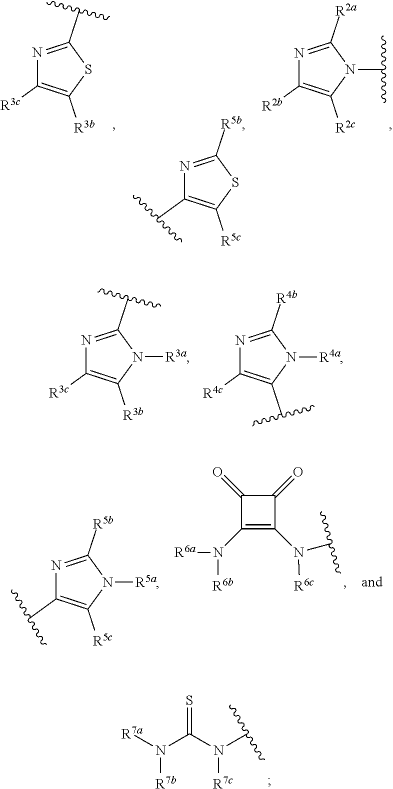

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

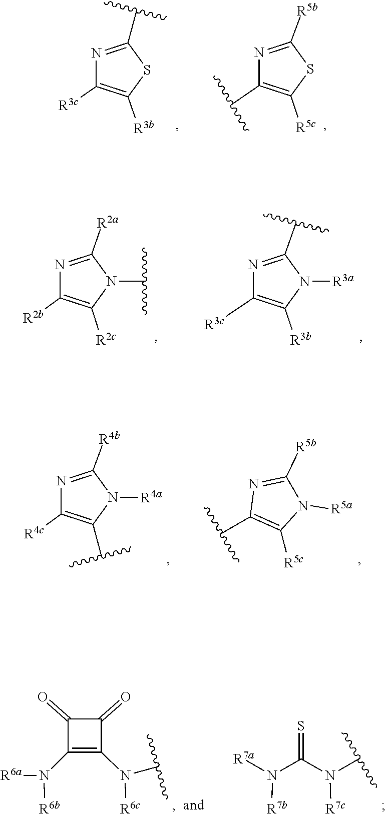

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₅-C₁₂ bridged cycloalkylenyl,

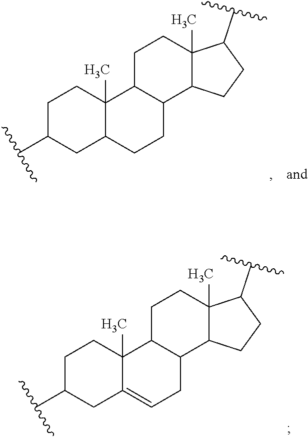 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

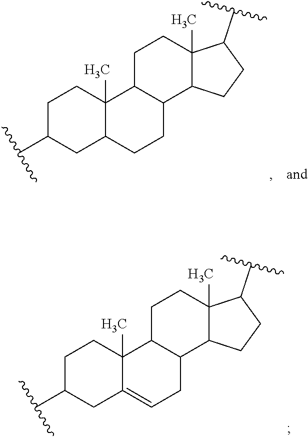 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IC:

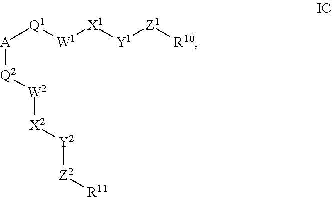

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

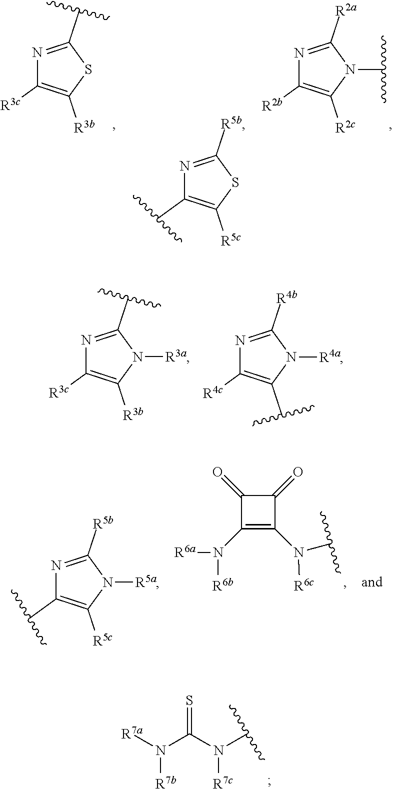

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

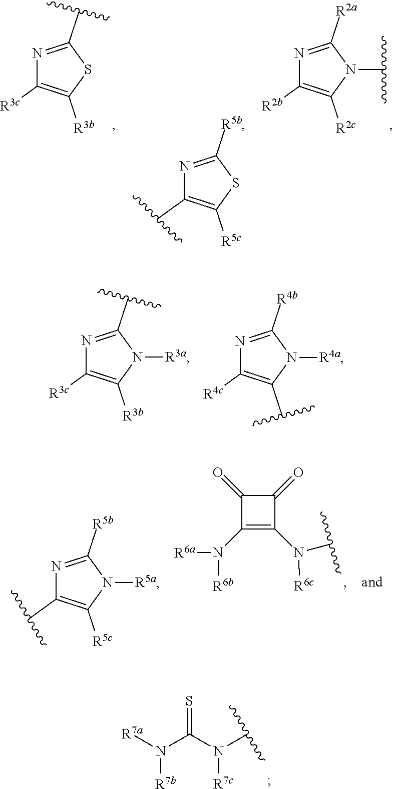

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₄-C₁₂ cycloalkylenyl,

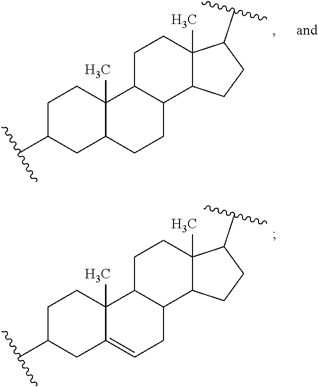 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is selected from the group consisting of —(CH₂)ₙ—, —O—, —S—, and —S—S—;
n is 0, 1, 2, 3, 4, 5, or 6;
Z² is of —(CH₂)ₚ—;
p is 0 or 1; and
R¹¹ is C₁-C₂₀ branched alkyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In some embodiments, the disclosure provides a compound of any one of Formulae IA, IB, IC, or a pharmaceutically acceptable salt or solvate thereof, wherein 21 is optionally substituted C₅-C₁₂ bridged cycloalkylenyl.

In some embodiments, the disclosure provides a compound of any one of Formulae IA, IB, IC, or a pharmaceutically acceptable salt or solvate thereof, wherein Z¹ is not adamantyl.

In one embodiment, the disclosure provides a compound of Formula ID:

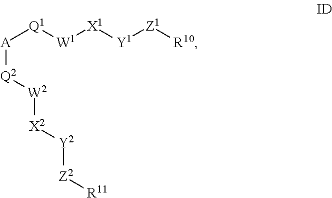

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

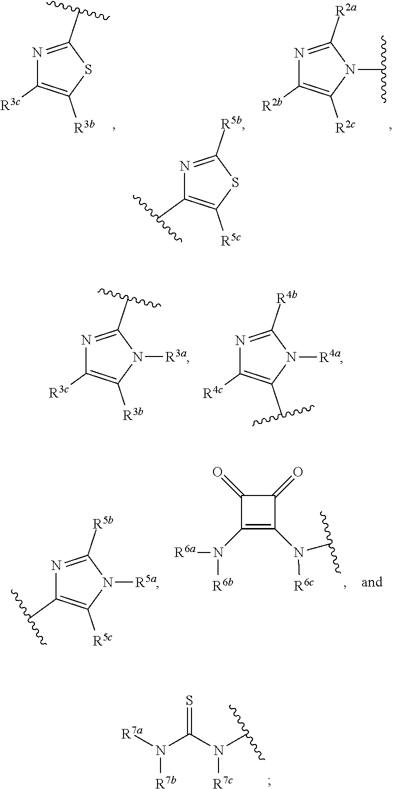

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7h}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

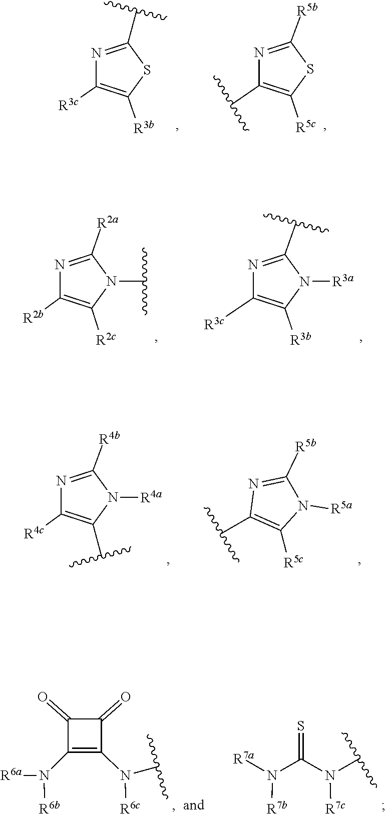

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is optionally substituted C₅-C₁₂ bridged cycloalkylenyl;
R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is —(CH₂)ₙ—;
n is 0, 1, 2, 3, 4, 5, or 6;
Z² is of —(CH₂)ₚ—;
p is 0 or 1; and
R¹¹ is C₁-C₂₀ branched alkyl.

In some embodiments, the disclosure provides a compound of Formula ID or a pharmaceutically acceptable salt or solvate thereof, wherein Z¹ is not adamantyl.

In one embodiment, the disclosure provides a compound of Formula I:

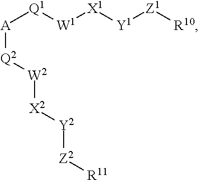

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl;
R¹ is selected from the group consisting of —OH,

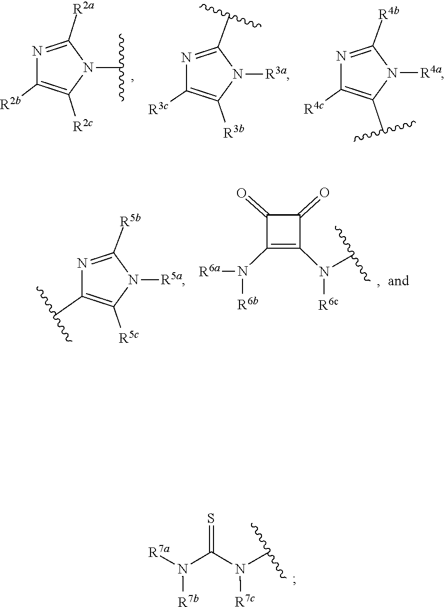

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is —NR^{9a}R^{9b};
R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of C₄-C₁₂ cycloalkylenyl,

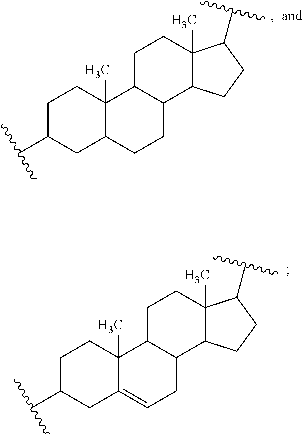 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, C₄-C₁₂ cycloalkylenyl,

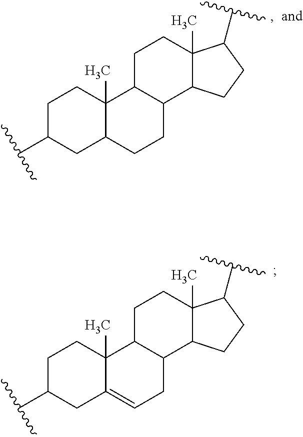 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl.

In another embodiment, the disclosure provides a compound of Formula II, III, VI, VI′, VI″, VI′″, VII, VII′, VII″, VII′″, VIII, VIII′, VIII″, VIII′″, IX, IX′, IX″, IX′″, X, X′, X″, X′″, XI, XI′, XI″, XI′″, XII, XII′, XII″, XII′″, XIII, XIII′, XIII″, XIII′″, XIV, XIV′, XIV″, XIV′″, XV, XV′, XV″, XV′″, XVI, XVI′, XVI″, XVI′″, XVII, XVIII, XVIII′, XIX, XX, or XXI, as described in PCT Publication WO2023122752A1:

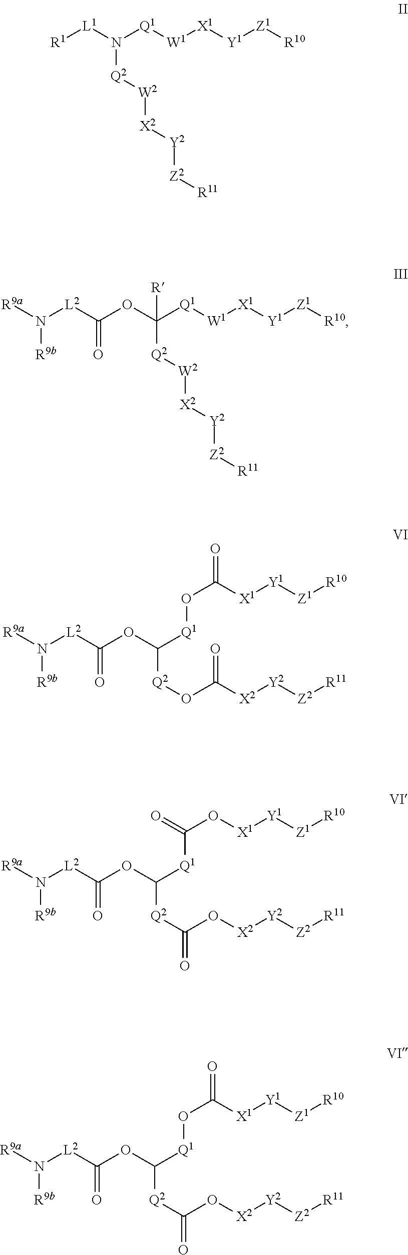

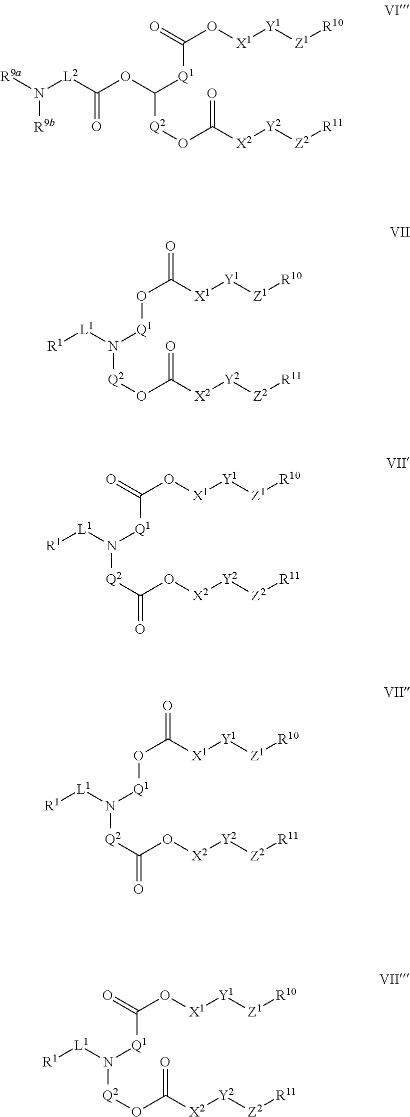

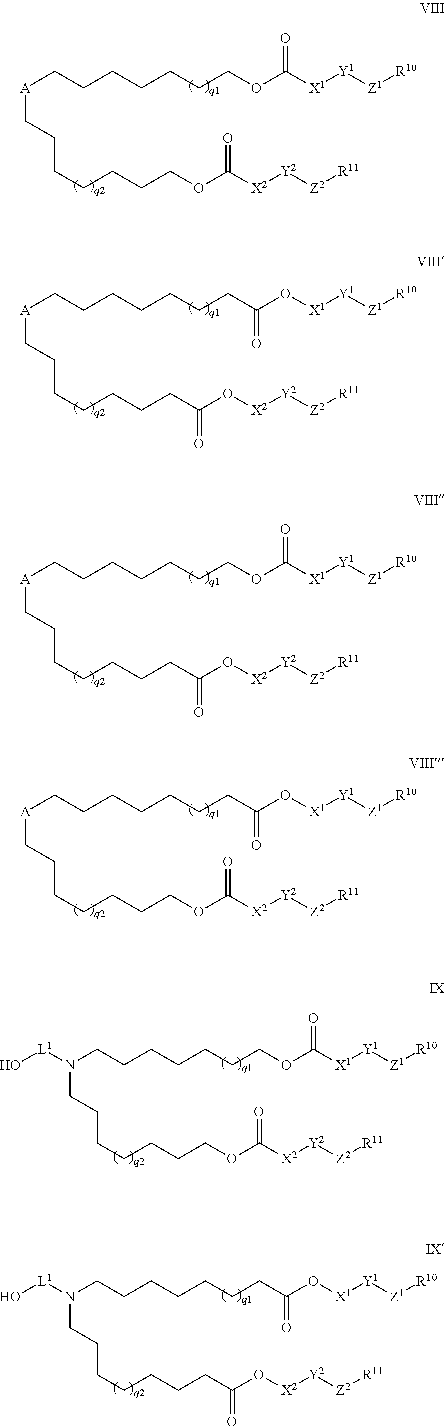

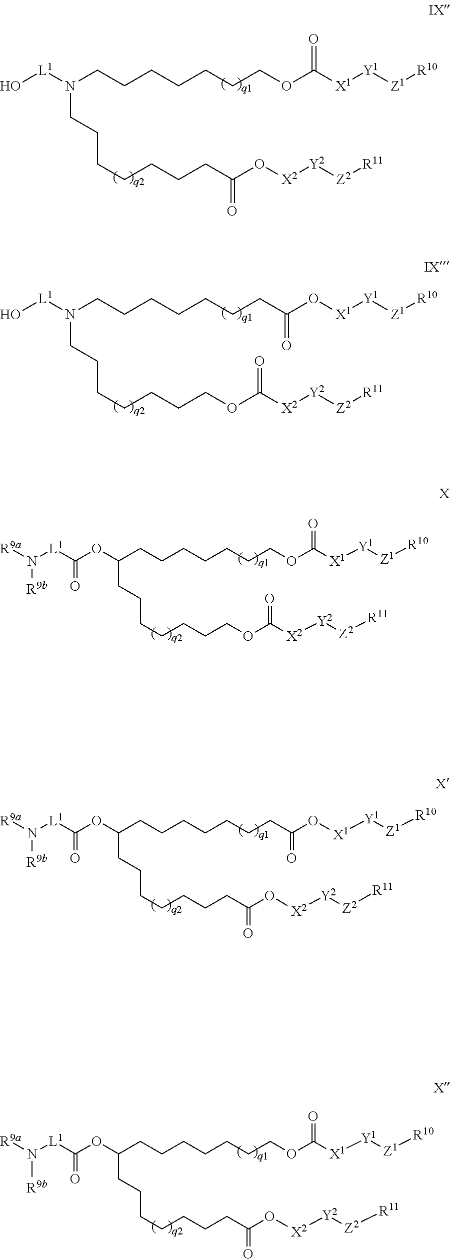

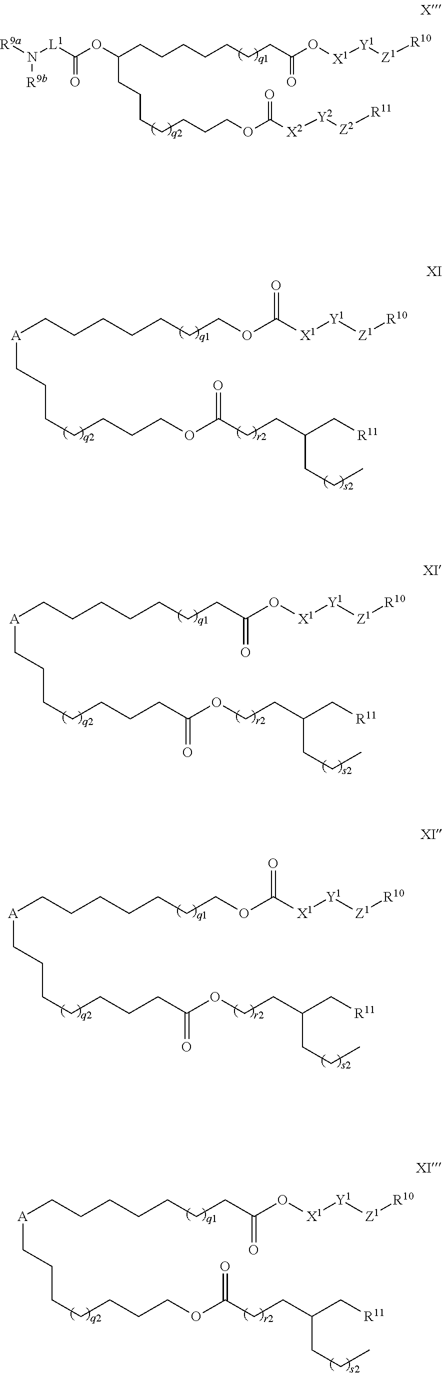

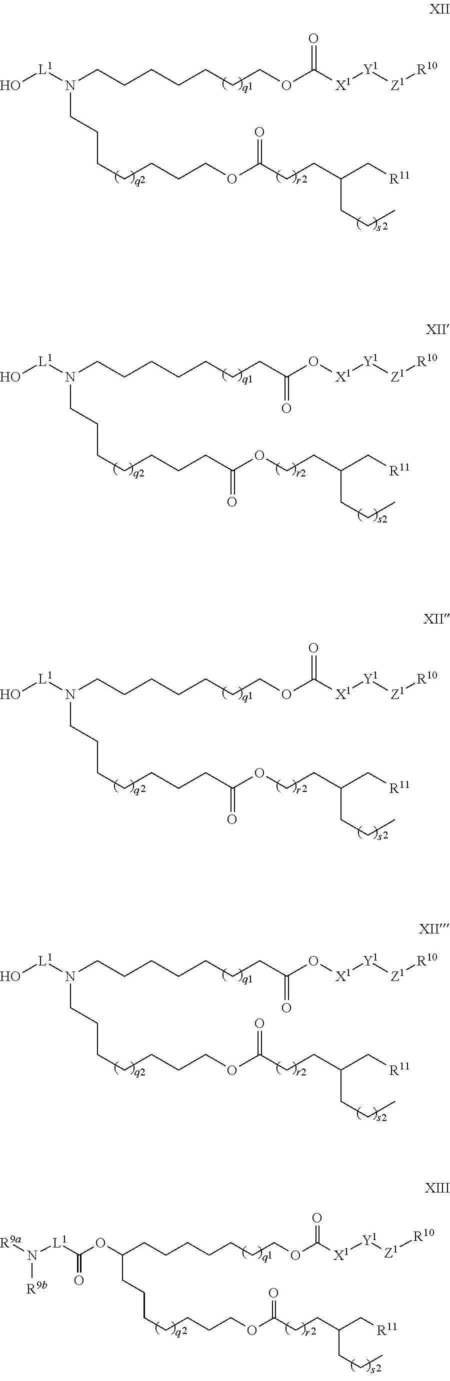

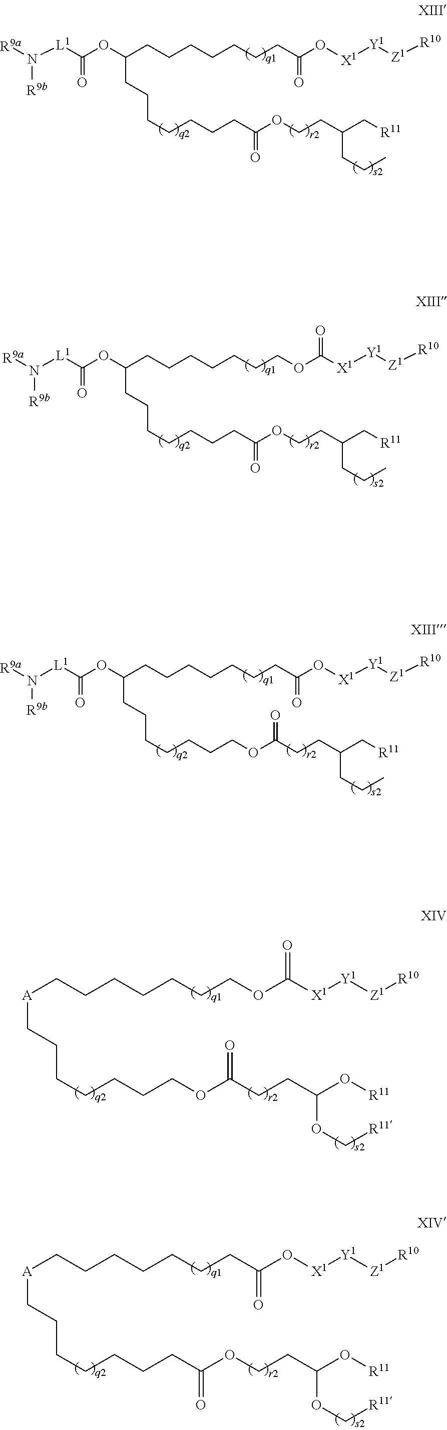

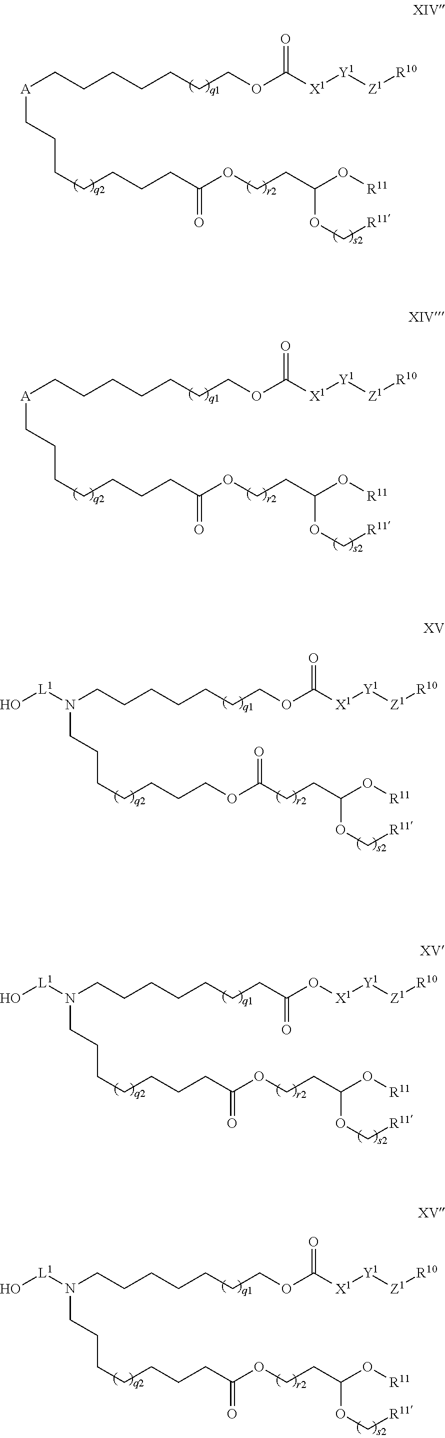

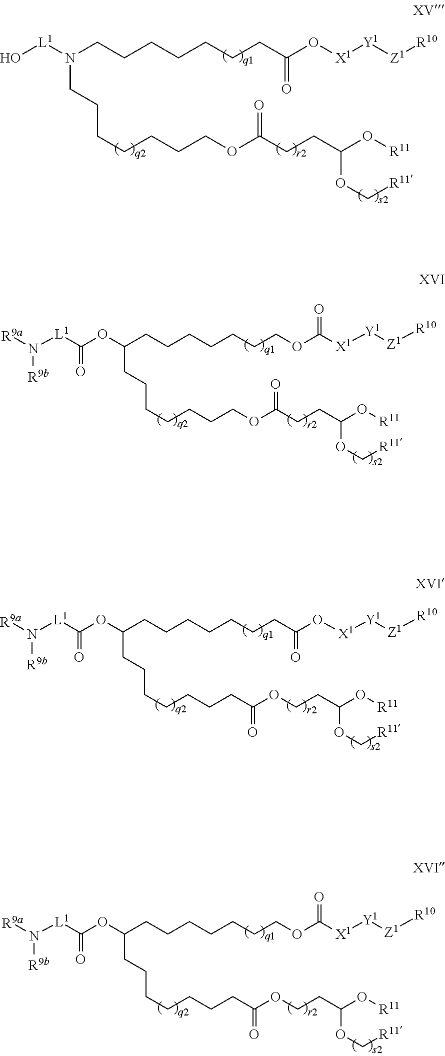

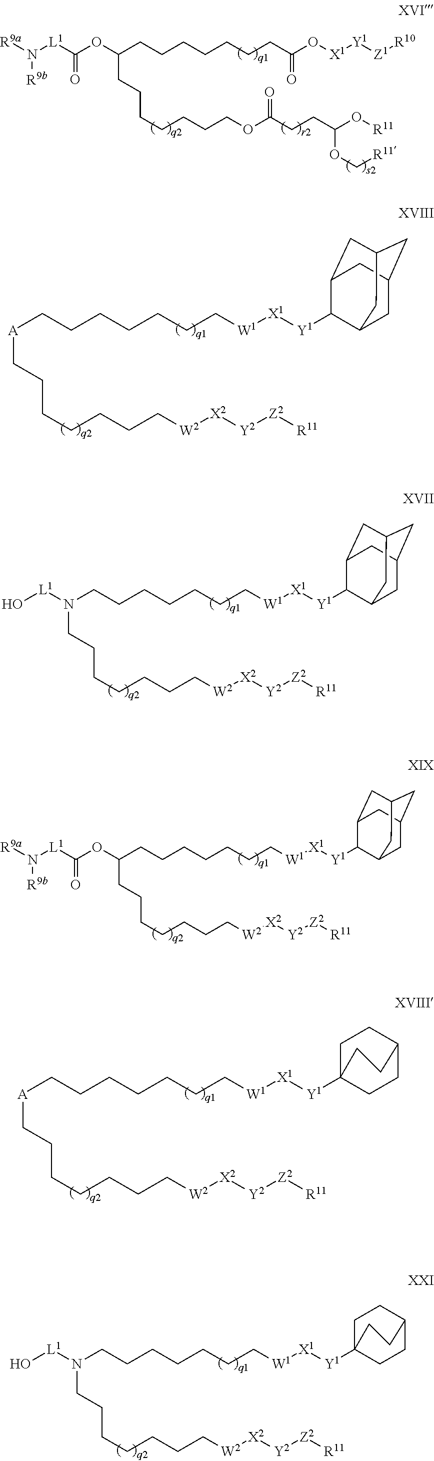

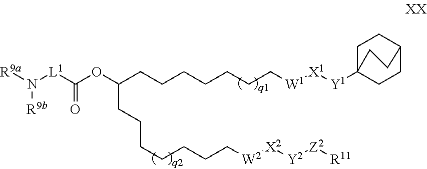

wherein each variable is as defined in Formula IA, Formula IB, Formula IC, Formula ID, Formula I, or below.

### L¹

In another embodiment, L¹ is selected from the group consisting of —CH₂CH₂—, —CH₂CH₂CH₂—, and CH₂CH₂CH₂CH₂—. In another embodiment, L¹ is —CH₂CH₂—. In another embodiment, L¹ is —CH₂CH₂CH₂—. In another embodiment, L¹ is CH₂CH₂CH₂CH₂—. In certain embodiments, L¹ is —(CH₂)₂₋₆—OC(═O)—. In some embodiments, L¹ is —(CH₂)₂—OC(═O)—.

### R¹

In some embodiments, R¹ is

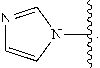 [SMILES: *n1ccnc1]

In another embodiment, R¹ is —OH. In some embodiments, R¹ is —N(R^{9a})(R^{9b}). In some embodiments, R¹ is —NMe₂. In some embodiments, R¹ is —NEt₂. In another embodiment, R¹ is

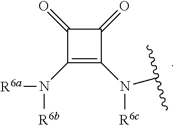

In another embodiment, R¹ is

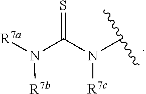

### L²

In another embodiment, L² is selected from the group consisting of —CH₂CH₂—, —CH₂CH₂CH₂—, and CH₂CH₂CH₂CH₂—. In another embodiment, L² is —CH₂CH₂—. In another embodiment, L² is —CH₂CH₂CH₂—. In another embodiment, L² is CH₂CH₂CH₂CH₂—.

### R⁸

In some embodiments, R⁸ is

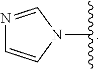 [SMILES: *n1ccnc1]

In another embodiment, R⁸ is —NR^{9a}R^{9b}. In some embodiments, R⁸ is —NMe₂. In some embodiments, R⁸ is —NEt₂. In another embodiment, R⁸ is —OH.
R^{9a}, R^{9b}

In another embodiment, R^{9a} and R^{9b} are independently selected from the group consisting of hydrogen and C₁-C₄ alkyl. In another embodiment, R^{9a} and R^{9b} are each methyl. In another embodiment, R^{9a} and R^{9b} are each ethyl.

### R′

In another embodiment, R′ is hydrogen. In some embodiments, R′ is C₁-C₆ alkyl.

### Q¹

In another embodiment, Q¹ is straight chain C₁-C₂₀ alkylenyl. In another embodiment, Q¹ is straight chain C₁-C₁₀ alkylenyl. In another embodiment, Q¹ is C₁-C₁₀ alkylenyl. In another embodiment, Q¹ is C₂-C₅ alkylenyl. Q¹ is C₆-C₉ alkylenyl. In another embodiment, Q¹ is selected from the group consisting of —CH₂CH₂—, CH₂CH₂CH₂—, CH₂(CH₂)₂CH₂—, CH₂(CH₂)₃CH₂—, CH₂(CH₂)₄CH₂—, CH₂(CH₂)₅CH₂—, CH₂(CH₂)₆CH₂—, CH₂(CH₂)₇CH₂—, and CH₂(CH₂)₅CH₂—. In another embodiment, Q¹ is —CH₂CH₂—. In another embodiment, Q¹ is CH₂CH₂CH₂—. In another embodiment, Q¹ is CH₂(CH₂)₂CH₂—. In another embodiment, Q¹ is CH₂(CH₂)₃CH₂—. In another embodiment, Q¹ is —CH₂CH₂—. In another embodiment, Q¹ is CH₂(CH₂)₄CH₂—. In another embodiment, Q¹ is CH₂(CH₂)₅CH₂—. In another embodiment, Q¹ is CH₂(CH₂)₆CH₂—. In another embodiment, Q¹ is CH₂(CH₂)₇CH₂—. In another embodiment, Q¹ is CH₂(CH₂)_{8.}CH₂—.

### W¹

In another embodiment, W¹ is selected from the group consisting of —C(═O)O—, —OC(═O)—, —C(═O)N(R^{12a})—, N(R^{12a})C(═O)—, —OC(═O)N(R^{12a})—, —N(R^{12a})C(═O)O—, and —OC(═O)O—. In another embodiment, W¹ is —C(═O)O—. In another embodiment, W¹ is —OC(═O)—. In another embodiment, W¹ is —C(═O)N(R^{12a})—. In another embodiment, W¹ is N(R^{12a})C(═O)—. In another embodiment, W¹ is —OC(═O)N(R^{12a})—. In another embodiment, W¹ is —N(R^{12a})C(═O)O—. In another embodiment, W¹ is —OC(═O)O—.

### X¹

In another embodiment, X² is optionally substituted C₁-C₁₅ alkylenyl. In another embodiment, X² is branched C₁-C₁₅ alkylenyl. In another embodiment, X¹ is a bond or C₁-C₁₅ alkylenyl. In another embodiment, X¹ is a bond. In another embodiment, X¹ is C₂-C₅ alkylenyl. In another embodiment, X¹ is C₆-C₉ alkylenyl. In another embodiment, X¹ is —CH₂—. In another embodiment, X² is —CH₂CH₂—. In another embodiment, X² is —CH₂CH₂CH₂—. In another embodiment, X² is —CH₂CH₂CH₂CH₂—. In another embodiment, X² is —CH₂CH₂CH₂CH₂CH₂—.

### Y¹

In another embodiment, Y¹ is selected from the group consisting of —(CH₂)ₘ—, —O—, —S—, and —S—S—. In another embodiment, Y¹ is —(CH₂)ₘ—. In some embodiments, Y¹ is —O—. In some embodiments, Y¹ is —S—. In another embodiment, Y¹ is —CH₂—. In another embodiment, Y² is —CH₂CH₂—.

m

In another embodiment, m is 0. In another embodiment, m is 1. In another embodiment, m is 2. In another embodiment, m is 3. In another embodiment, m is 4. In another embodiment, m is 5. In another embodiment, m is 6.

n

In another embodiment, n is 0. In another embodiment, n is 1. In another embodiment, n is 2. In another embodiment, n is 3. In another embodiment, n is 4. In another embodiment, n is 5. In another embodiment, n is 6.

p

In another embodiment, p is 0. In another embodiment, p is 1.

### Z¹

In another embodiment, Z¹ is selected from the group consisting of C₄-C₁₂ cycloalkylenyl,

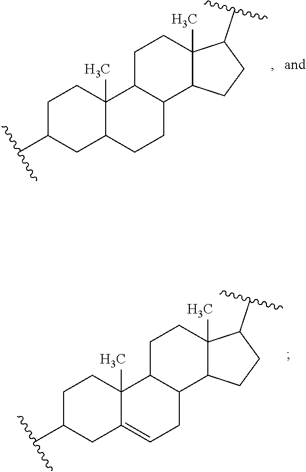 [SMILES: *C1CCC2(C)C(CCC3C4CCC(*)C4(C)CCC32)C1 ; *C1CCC2(C)C(=CCC3C2CCC2(C)C(*)CCC32)C1]

In certain embodiments, Z¹ is optionally substituted.

In another embodiment, Z¹ is

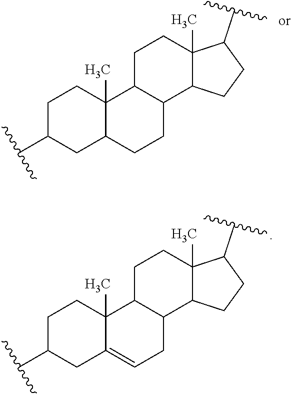 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, Z¹ is C₄-C₁₂ cycloalkylenyl. In another embodiment, Z¹ is a monocyclic C₄-C₈ cycloalkylenyl. In another embodiment, Z¹ is a monocyclic C₄-C₆ cycloalkylenyl. In another embodiment, Z¹ is a monocyclic C₄ cycloalkylenyl. In another embodiment, Z¹ is a monocyclic C₅ cycloalkylenyl. In another embodiment, Z¹ is a monocyclic C₆ cycloalkylenyl.

In another embodiment, Z¹ is an optionally substituted bridged bicyclic or multicyclic cycloalkylenyl. In some embodiments, Z¹ is optionally substituted C₅-C₁₂ bridged cycloalkylenyl. In some embodiments, Z¹ is optionally substituted C₆-C₁₀ bridged cycloalkylenyl. In some embodiments, Z¹ is a optionally substituted C₅-C₁₀ bridged cycloalkylenyl. selected from the group consisting of adamantyl, cubanyl, bicyclo[2.2.1]heptyl, bicyclo[2.2.2]octyl, bicyclo[1.1.1]pentyl, bicyclo[3.2.1]octyl, and bicyclo[3.1.1]heptyl.

In another embodiment, 21 is selected from the group consisting of:

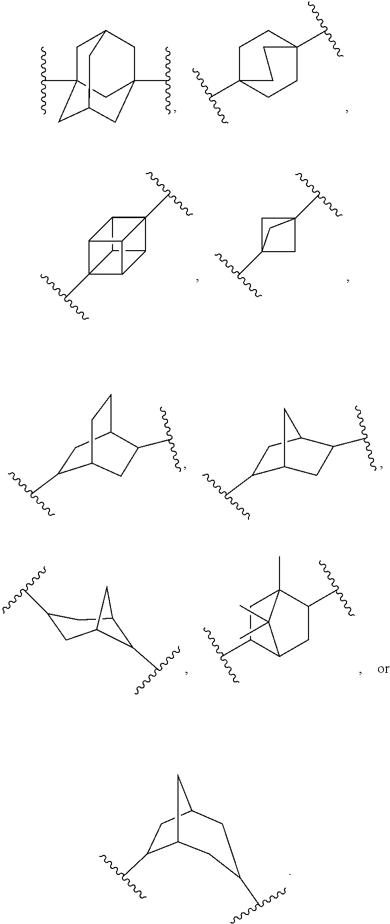

In another embodiment, Z¹ is selected from the group consisting of:

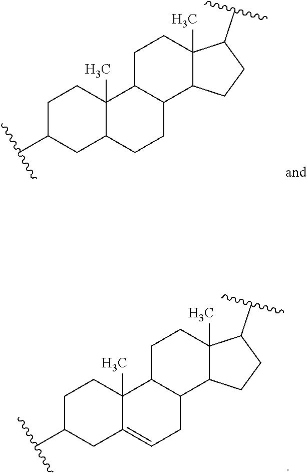 [SMILES: *C1CCC2(C)C(CCC3C4CCC(*)C4(C)CCC32)C1 ; *C1CCC2(C)C(=CCC3C2CCC2(C)C(*)CCC32)C1]

### R¹⁰

In another embodiment, R¹⁰ is hydrogen.

In another embodiment, R¹⁰ is C₁C₁₀ alkyl. In another embodiment, R¹⁰ is C₃C₇ alkyl. In another embodiment, R¹⁰ is C₄C₆ alkyl. In another embodiment, R¹⁰ is C₄. In another embodiment, R¹⁰ is C₅. In another embodiment, R¹⁰ is C₆.

In another embodiment, R¹⁰ is C₂-C₁₂ alkenyl. In another embodiment, R¹⁰ is C₆-C₁₂ alkenyl. In another embodiment, R¹⁰ is C₂-C₈ alkenyl.

### R¹¹

In another embodiment, R¹¹ is C₁-C₁₀ alkyl. In another embodiment, R¹¹ is optionally substituted C₁-C₂₀ alkyl. In another embodiment, R¹¹ is optionally substituted branched C₁-C₂₀ alkyl. In another embodiment, R¹¹ is optionally substituted C₁-C₁₅ alkyl. In another embodiment, R¹¹ is optionally substituted C₁-C₁₅ branched alkyl. In another embodiment, R¹¹ is optionally substituted C₁₀-C₁₅ alkyl. In another embodiment, R¹¹ is optionally substituted C₁₀-C₁₅ branched alkyl. In another embodiment, R¹¹ is selected from the group consisting of CH₃, —CH₂CH₃, and —CH₂CH₂CH₃. In another embodiment, R¹¹ is selected from the group consisting of CH₂(CH₂)₂CH₃, CH₂(CH₂)₃CH₃, CH₂(CH₂)₄CH₃, —CH₂(CH₂)₅CH₃, —CH₂(CH₂)₆CH₃, CH₂(CH₂)₇CH₃, and —CH₂(CH₂)₈CH₃. In another embodiment, R¹¹ is CH₃. In another embodiment, R¹¹ is —CH₂CH₃. In another embodiment, R¹¹ is —CH₂CH₂CH₃. In another embodiment, R¹¹ is CH₂(CH₂)₂CH₃. In another embodiment, R¹¹ is CH₂(CH₂)₃CH₃. In another embodiment, R¹¹ is CH₂(CH₂)₄CH₃. In another embodiment, R¹¹ is —CH₂(CH₂)₅CH₃. In another embodiment, R¹¹ is CH₂(CH₂)₆CH₃. In another embodiment, R¹¹ is CH₂(CH₂)₇CH₃. In another embodiment, R¹¹ is —CH₂(CH₂)₈CH₃.

In another embodiment, R¹¹ is C₂-C₁₀ alkenyl. In another embodiment, R¹¹ is C₂-C₁₂ alkenyl. In another embodiment, R¹¹ is C₆-C₁₂ alkenyl. In another embodiment, R¹¹ is C₂-C₈ alkenyl.

In another embodiment, the disclosure provides a compound of any one of Formulae IA, IB, IC, or I-XXI or a pharmaceutically acceptable salt or solvate thereof, wherein R¹¹ is hydrogen.

### Q²

In another embodiment, Q² is straight chain C₁-C₂₀ alkylenyl. In another embodiment, Q² is straight chain C₁-C₁₀ alkylenyl. In another embodiment, Q² is C₂-C₁₀ alkylenyl. In another embodiment, Q² is selected from the group consisting of —CH₂CH₂—, CH₂CH₂CH₂—, CH₂(CH₂)₂CH₂—, CH₂(CH₂)₃CH₂—, CH₂(CH₂)₄CH₂—, CH₂(CH₂)₅CH₂—, CH₂(CH₂)₆CH₂—, CH₂(CH₂)₇CH₂—, and CH₂(CH₂)_{8.}CH₂—. In another embodiment, Q² is —CH₂CH₂—. In another embodiment, Q² is CH₂CH₂CH₂—. In another embodiment, Q² is CH₂(CH₂)₃CH₂—. In another embodiment, Q² is CH₂(CH₂)₄CH₂—. In another embodiment, Q² is CH₂(CH₂)₅CH₂—. In another embodiment, Q² is CH₂(CH₂)₆CH₂—. In another embodiment, Q² is CH₂(CH₂)₇CH₂—. In another embodiment, Q² is CH₂(CH₂)_{8.}CH₂—.

### W²

In another embodiment, W² is selected from the group consisting of —C(═O)O— and —OC(═O)—. In another embodiment, W² is —C(═O)O—. In another embodiment, W² is —OC(═O)—.

### X²

In another embodiment, X² is optionally substituted C₁-C₁₅ alkylenyl. In another embodiment, X² is C₁-C₁₅ branched alkylenyl. In another embodiment, X² is C₁-C₆ alkylenyl or a bond. In another embodiment, X² is C₂-C₄ alkylenyl. In another embodiment, X² is C₃-C₅ alkylenyl. In another embodiment, X² is selected from the group consisting of —CH₂CH₂—, CH₂CH₂CH₂—, CH₂(CH₂)₂CH₂—, CH₂(CH₂)₃CH₂—, and CH₂(CH₂)₄CH₂—. In another embodiment, X² is —CH₂—. In another embodiment, X² is a bond. In another embodiment, X² is branched C₁-C₁₅ alkylenyl, wherein one or more methylene linkages of X² are optionally and independently replaced with a group selected from —O—, —CH═CH—, —S— and C₃-C₆ cycloalkylenyl.

### Y²

In another embodiment, Y² is selected from the group consisting of —(CH₂)ₘ— and —S—. In another embodiment, Y² is —(CH₂)ₘ—. In another embodiment, Y² is —S—.

### Z²

In another embodiment, Z² is —(CH₂)ₚ—. In another embodiment, Z² is —CH₂—. In another embodiment, Z² is —CH₂CH₂—. In another embodiment, Z² is C₄-C₁₂ cycloalkylenyl. In another embodiment, Z² is a monocyclic C₄-C₈ cycloalkylenyl. In certain embodiments, Z² is optionally substituted.

In another embodiment, Z² is an optionally substituted bridged bicyclic or multicyclic cycloalkylenyl. In some embodiments, Z² is optionally substituted C₅-C₁₂ bridged cycloalkylenyl. In some embodiments, Z² is optionally substituted C₆-C₁₀ bridged cycloalkylenyl. In some embodiments, Z² is an optionally substituted C₅-C₁₀ bridged cycloalkylenyl. selected from the group consisting of adamantyl, cubanyl, bicyclo[2.2.1]heptyl, bicyclo[2.2.2]octyl, bicyclo[1.1.1]pentyl, bicyclo[3.2.1]octyl, and bicyclo[3.1.1]heptyl.

In another embodiment, Z² is selected from the group consisting of:

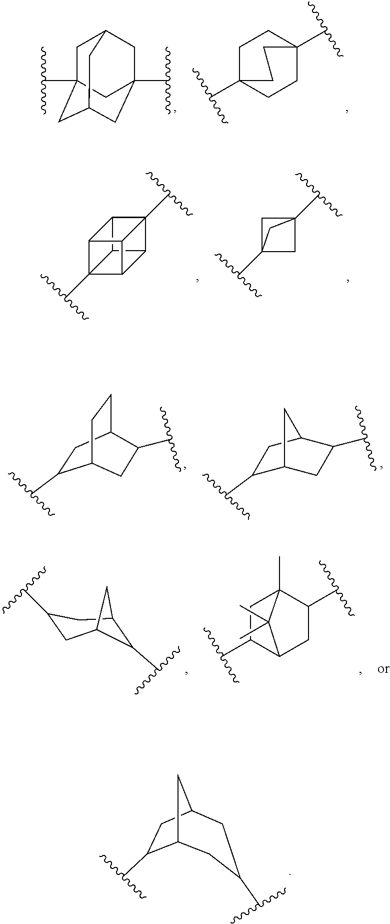

In another embodiment, Z² is selected from the group consisting of:

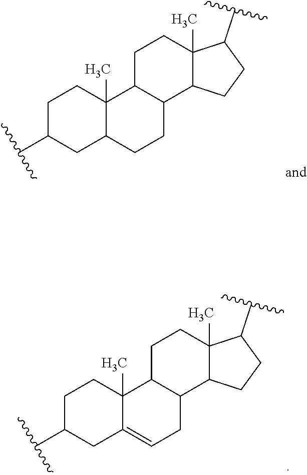 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, the disclosure provides a compound selected from any one of more of the compounds of Table (III), or a pharmaceutically acceptable salt or solvate thereof.

TABLE (III)
Non-Limiting Examples of Ionizable Lipids with a Constrained Arm

| # | Structure |
|---|---|
| C1 | 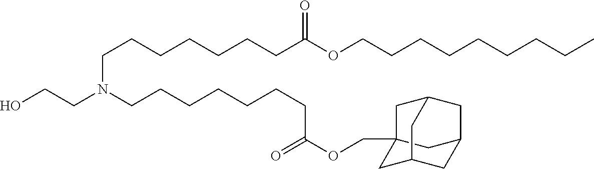 [SMILES: CCCCCCCCCOC(=O)CCCCCCCN(CCO)CCCCCCCC(=O)OCC12CC3CC(CC(C3)C1)C2] |
| C2 | 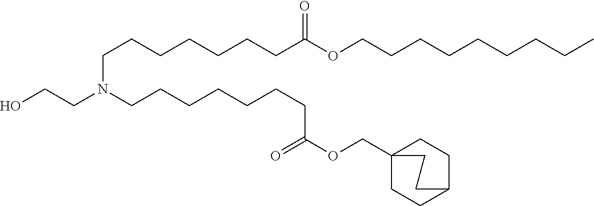 [SMILES: CCCCCCCCCOC(=O)CCCCCCCN(CCO)CCCCCCCC(=O)OCC12CCC(CC1)CC2] |
| C3 | 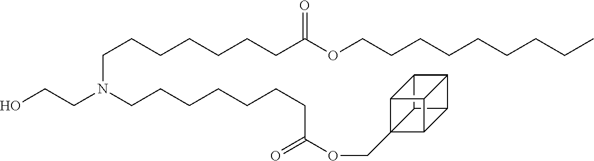 [SMILES: CCCCCCCCCOC(=O)CCCCCCCN(CCO)CCCCCCCC(=O)OCC12C3C4C5C3C1C5C42] |
| C4 | 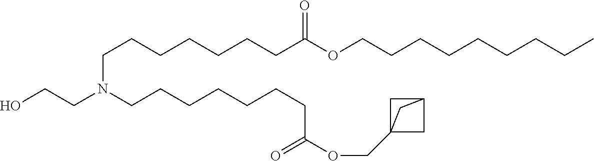 [SMILES: CCCCCCCCCOC(=O)CCCCCCCN(CCO)CCCCCCCC(=O)OCC12CC(C1)C2] |
| C5 | 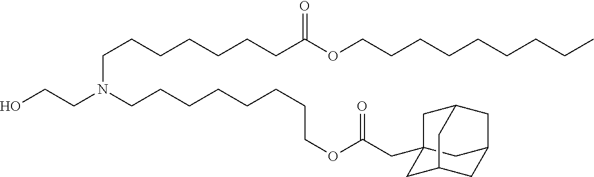 [SMILES: CCCCCCCCCOC(=O)CCCCCCCN(CCO)CCCCCCCCOC(=O)CC12CC3CC(CC(C3)C1)C2] |
| C6 | 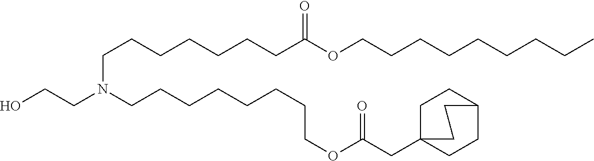 [SMILES: CCCCCCCCCOC(=O)CCCCCCCN(CCO)CCCCCCCCOC(=O)CC12CCC(CC1)CC2] |
| C7 | 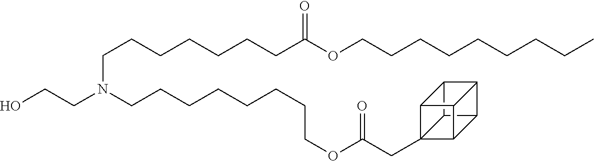 [SMILES: CCCCCCCCCOC(=O)CCCCCCCN(CCO)CCCCCCCCOC(=O)CC12C3C4C5C3C1C5C42] |
| C8 | 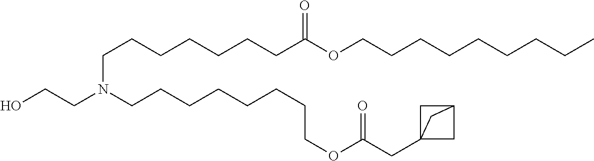 [SMILES: CCCCCCCCCOC(=O)CCCCCCCN(CCO)CCCCCCCCOC(=O)CC12CC(C1)C2] |
| C9 | 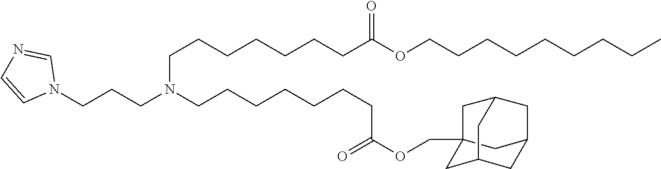 [SMILES: CCCCCCCCCOC(=O)CCCCCCCN(CCCCCCCC(=O)OCC12CC3CC(CC(C3)C1)C2)CCCn1ccnc1] |
| C10 | 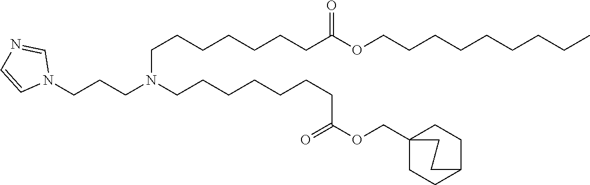 [SMILES: CCCCCCCCCOC(=O)CCCCCCCN(CCCCCCCC(=O)OCC12CCC(CC1)CC2)CCCn1ccnc1] |
| C11 | 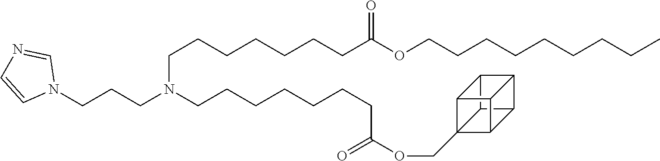 [SMILES: CCCCCCCCCOC(=O)CCCCCCCN(CCCCCCCC(=O)OCC12C3C4C5C3C1C5C42)CCCn1ccnc1] |
| C12 | 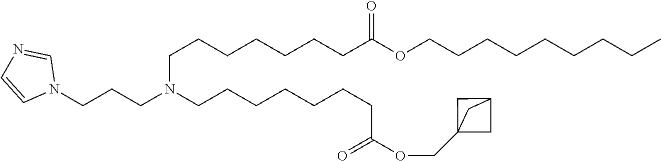 [SMILES: CCCCCCCCCOC(=O)CCCCCCCN(CCCCCCCC(=O)OCC12CC(C1)C2)CCCn1ccnc1] |
| C13 | 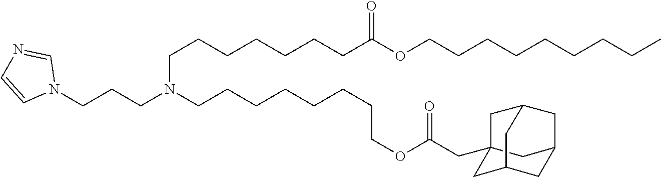 [SMILES: CCCCCCCCCOC(=O)CCCCCCCN(CCCCCCCCOC(=O)CC12CC3CC(CC(C3)C1)C2)CCCn1ccnc1] |
| C14 | 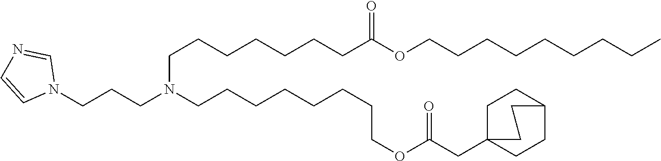 [SMILES: CCCCCCCCCOC(=O)CCCCCCCN(CCCCCCCCOC(=O)CC12CCC(CC1)CC2)CCCn1ccnc1] |
| C15 | 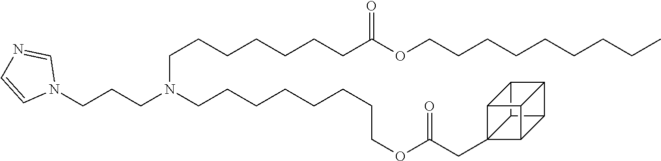 [SMILES: CCCCCCCCCOC(=O)CCCCCCCN(CCCCCCCCOC(=O)CC12C3C4C5C3C1C5C42)CCCn1ccnc1] |
| C16 | 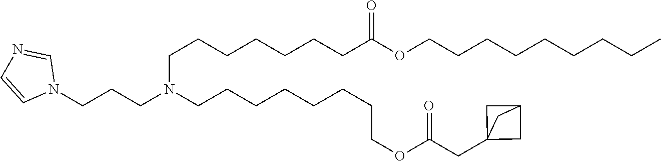 [SMILES: CCCCCCCCCOC(=O)CCCCCCCN(CCCCCCCCOC(=O)CC12CC(C1)C2)CCCn1ccnc1] |
| C17 | 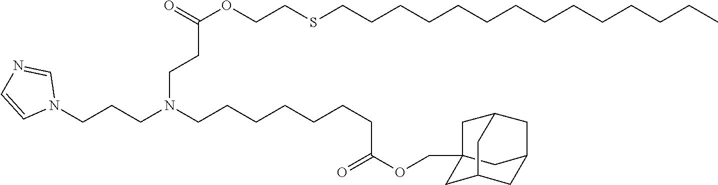 [SMILES: CCCCCCCCCCCCCCSCCOC(=O)CCN(CCCCCCCC(=O)OCC12CC3CC(CC(C3)C1)C2)CCCn1ccnc1] |
| C18 | 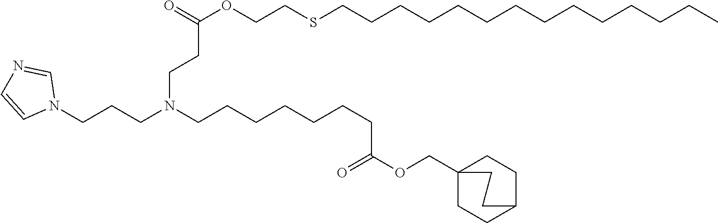 [SMILES: CCCCCCCCCCCCCCSCCOC(=O)CCN(CCCCCCCC(=O)OCC12CCC(CC1)CC2)CCCn1ccnc1] |
| C19 | 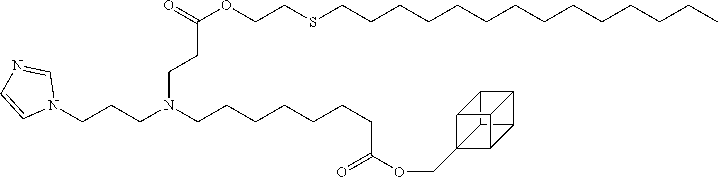 [SMILES: CCCCCCCCCCCCCCSCCOC(=O)CCN(CCCCCCCC(=O)OCC12C3C4C5C3C1C5C42)CCCn1ccnc1] |
| C20 | 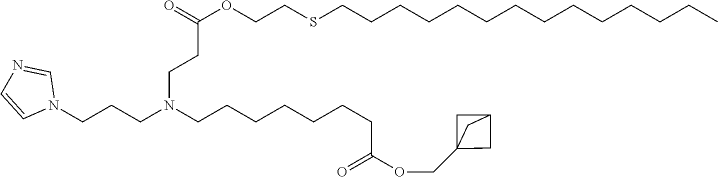 [SMILES: CCCCCCCCCCCCCCSCCOC(=O)CCN(CCCCCCCC(=O)OCC12CC(C1)C2)CCCn1ccnc1] |
| C21 | 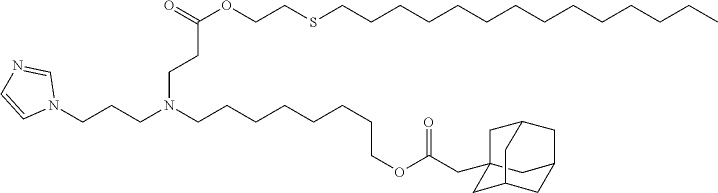 [SMILES: CCCCCCCCCCCCCCSCCOC(=O)CCN(CCCCCCCCOC(=O)CC12CC3CC(CC(C3)C1)C2)CCCn1ccnc1] |
| C22 | 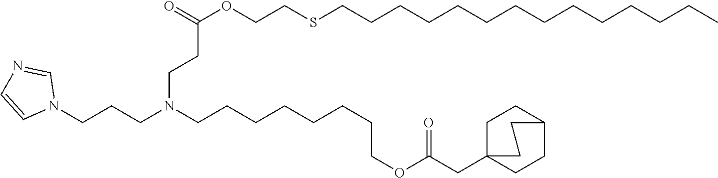 [SMILES: CCCCCCCCCCCCCCSCCOC(=O)CCN(CCCCCCCCOC(=O)CC12CCC(CC1)CC2)CCCn1ccnc1] |
| C23 | 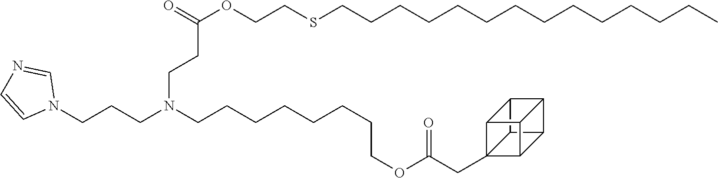 [SMILES: CCCCCCCCCCCCCCSCCOC(=O)CCN(CCCCCCCCOC(=O)CC12C3C4C5C3C1C5C42)CCCn1ccnc1] |
| C24 | 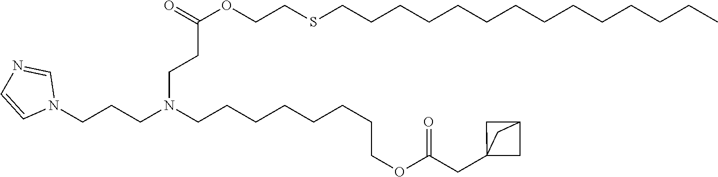 [SMILES: CCCCCCCCCCCCCCSCCOC(=O)CCN(CCCCCCCCOC(=O)CC12CC(C1)C2)CCCn1ccnc1] |
| C25 | 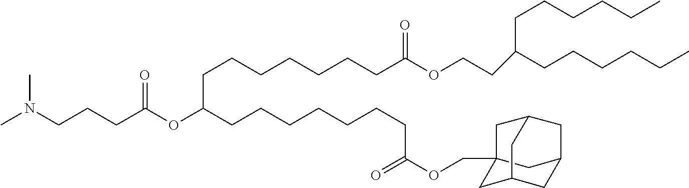 [SMILES: CCCCCCC(CCCCCC)CCOC(=O)CCCCCCCC(CCCCCCCC(=O)OCC12CC3CC(CC(C3)C1)C2)OC(=O)CCCN(C)C] |
| C26 | 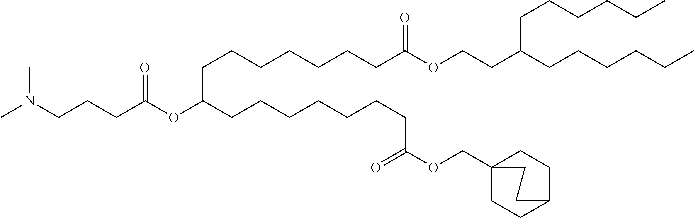 [SMILES: CCCCCCC(CCCCCC)CCOC(=O)CCCCCCCC(CCCCCCCC(=O)OCC12CCC(CC1)CC2)OC(=O)CCCN(C)C] |
| C27 | 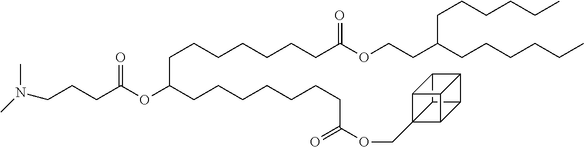 [SMILES: CCCCCCC(CCCCCC)CCOC(=O)CCCCCCCC(CCCCCCCC(=O)OCC12C3C4C5C3C1C5C42)OC(=O)CCCN(C)C] |
| C28 | 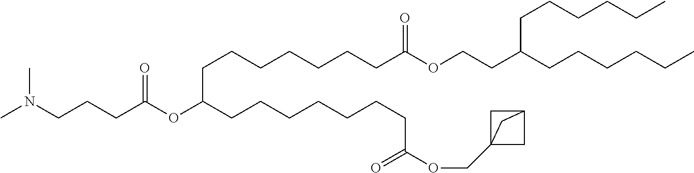 [SMILES: CCCCCCC(CCCCCC)CCOC(=O)CCCCCCCC(CCCCCCCC(=O)OCC12CC(C1)C2)OC(=O)CCCN(C)C] |
| C29 | 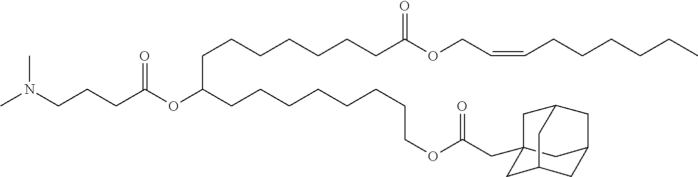 [SMILES: CCCCCC/C=C\COC(=O)CCCCCCCC(CCCCCCCCOC(=O)CC12CC3CC(CC(C3)C1)C2)OC(=O)CCCN(C)C] |
| C30 | 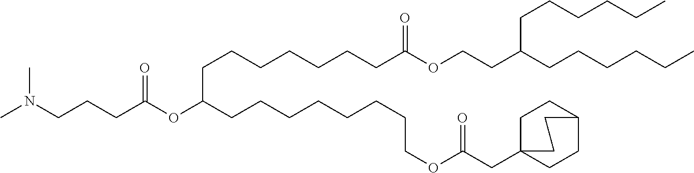 [SMILES: CCCCCCC(CCCCCC)CCOC(=O)CCCCCCCC(CCCCCCCCOC(=O)CC12CCC(CC1)CC2)OC(=O)CCCN(C)C] |
| C31 | 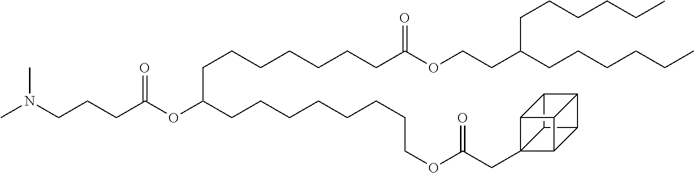 [SMILES: CCCCCCC(CCCCCC)CCOC(=O)CCCCCCCC(CCCCCCCCOC(=O)CC12C3C4C5C3C1C5C42)OC(=O)CCCN(C)C] |
| C32 | 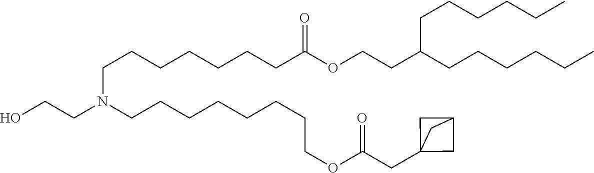 [SMILES: CCCCCCC(CCCCCC)CCOC(=O)CCCCCCCN(CCO)CCCCCCCCOC(=O)CC12CC(C1)C2] |
| C33 | 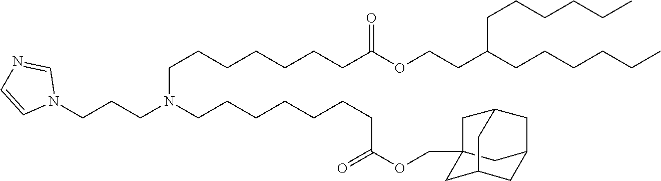 [SMILES: CCCCCCC(CCCCCC)CCOC(=O)CCCCCCCN(CCCCCCCC(=O)OCC12CC3CC(CC(C3)C1)C2)CCCn1ccnc1] |
| C34 | 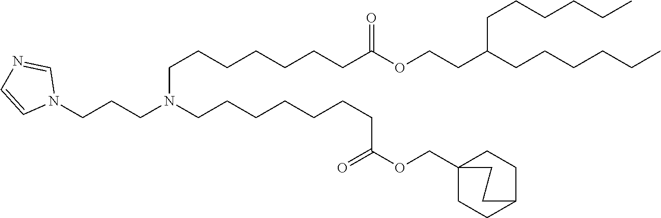 [SMILES: CCCCCCC(CCCCCC)CCOC(=O)CCCCCCCN(CCCCCCCC(=O)OCC12CCC(CC1)CC2)CCCn1ccnc1] |
| C35 | 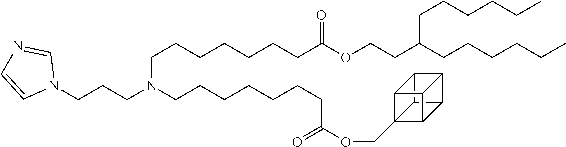 [SMILES: CCCCCCC(CCCCCC)CCOC(=O)CCCCCCCN(CCCCCCCC(=O)OCC12C3C4C5C3C1C5C42)CCCn1ccnc1] |
| C36 | 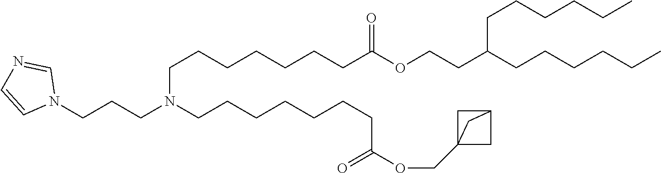 [SMILES: CCCCCCC(CCCCCC)CCOC(=O)CCCCCCCN(CCCCCCCC(=O)OCC12CC(C1)C2)CCCn1ccnc1] |
| C37 | 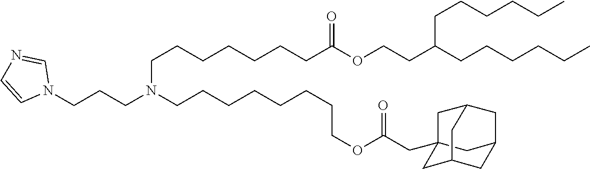 [SMILES: CCCCCCC(CCCCCC)CCOC(=O)CCCCCCCN(CCCCCCCCOC(=O)CC12CC3CC(CC(C3)C1)C2)CCCn1ccnc1] |
| C38 | 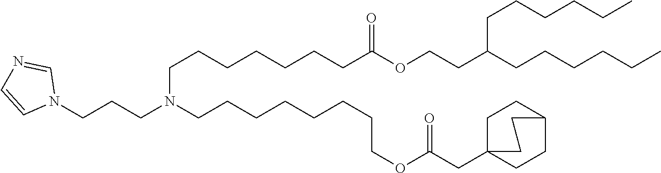 [SMILES: CCCCCCC(CCCCCC)CCOC(=O)CCCCCCCN(CCCCCCCCOC(=O)CC12CCC(CC1)CC2)CCCn1ccnc1] |
| C39 | 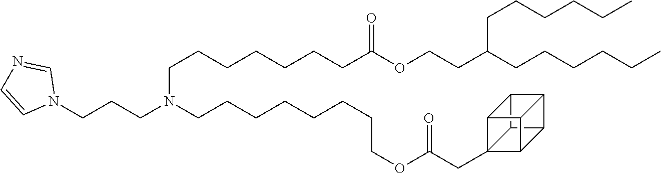 [SMILES: CCCCCCC(CCCCCC)CCOC(=O)CCCCCCCN(CCCCCCCCOC(=O)CC12C3C4C5C3C1C5C42)CCCn1ccnc1] |
| C40 | 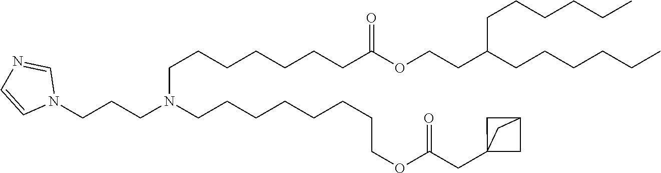 [SMILES: CCCCCCC(CCCCCC)CCOC(=O)CCCCCCCN(CCCCCCCCOC(=O)CC12CC(C1)C2)CCCn1ccnc1] |
| C41 | 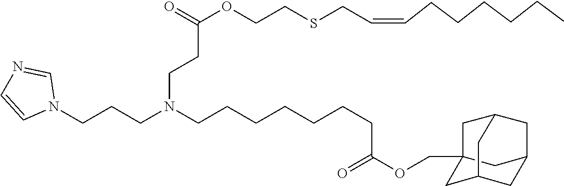 [SMILES: CCCCCC/C=C\CSCCOC(=O)CCN(CCCCCCCC(=O)OCC12CC3CC(CC(C3)C1)C2)CCCn1ccnc1] |
| C42 | 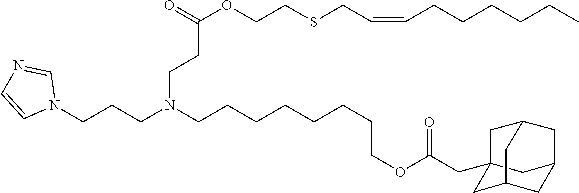 [SMILES: CCCCCC/C=C\CSCCOC(=O)CCN(CCCCCCCCOC(=O)CC12CC3CC(CC(C3)C1)C2)CCCn1ccnc1] |
| C43 | 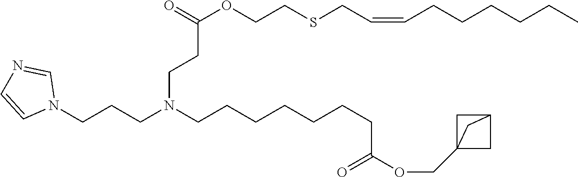 [SMILES: CCCCCC/C=C\CSCCOC(=O)CCN(CCCCCCCC(=O)OCC12CC(C1)C2)CCCn1ccnc1] |
| C44 | 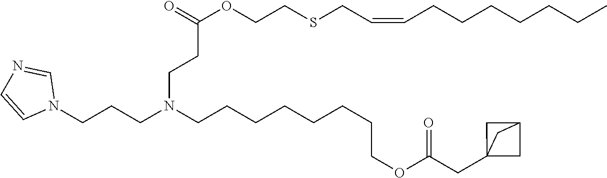 [SMILES: CCCCCCC/C=C\CSCCOC(=O)CCN(CCCCCCCCOC(=O)CC12CC(C1)C2)CCCn1ccnc1] |
| C45 | 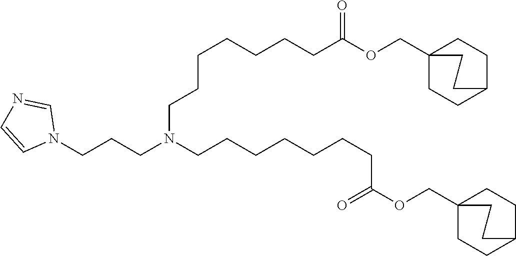 [SMILES: O=C(CCCCCCCN(CCCCCCCC(=O)OCC12CCC(CC1)CC2)CCCn1ccnc1)OCC12CCC(CC1)CC2] |
| C46 | 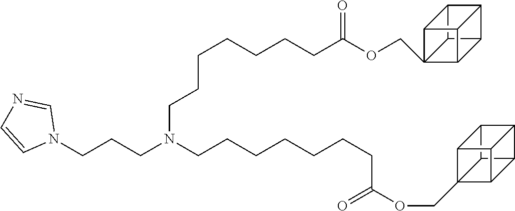 [SMILES: O=C(CCCCCCCN(CCCCCCCC(=O)OCC12C3C4C5C3C1C5C42)CCCn1ccnc1)OCC12C3C4C5C3C1C5C42] |
| C47 | 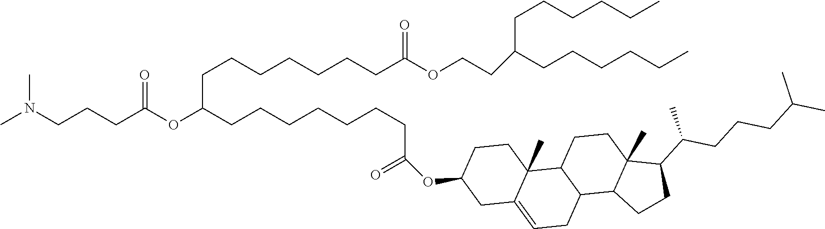 |
| C48 | 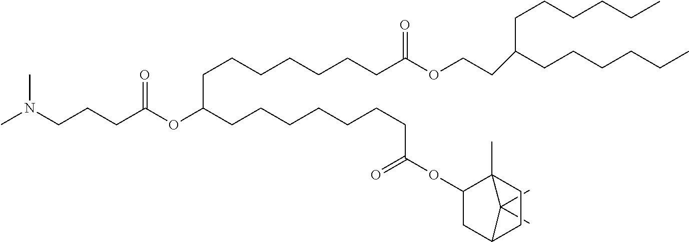 [SMILES: CCCCCCC(CCCCCC)CCOC(=O)CCCCCCCC(CCCCCCCC(=O)OC1CC2CCC1(C)C2(C)C)OC(=O)CCCN(C)C] |
| C49 | 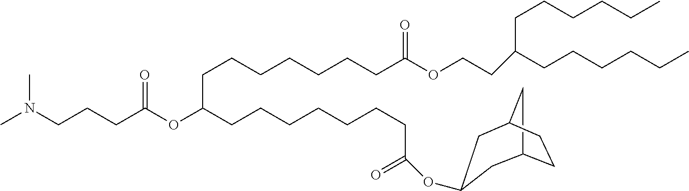 [SMILES: CCCCCCC(CCCCCC)CCOC(=O)CCCCCCCC(CCCCCCCC(=O)OC1CC2CCC(C2)C1)OC(=O)CCCN(C)C] |
| C50 | 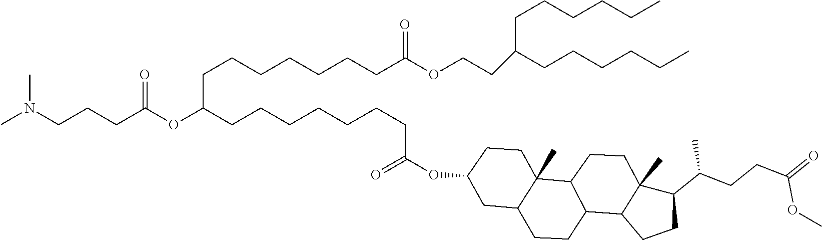 |
| C51 | 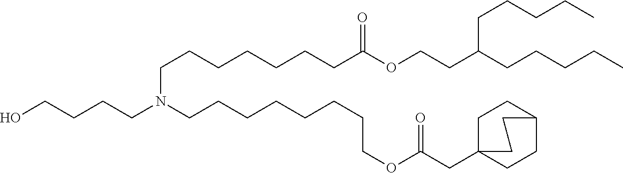 [SMILES: CCCCCC(CCCCC)CCOC(=O)CCCCCCCN(CCCCO)CCCCCCCCOC(=O)CC12CCC(CC1)CC2] |
| C52 | 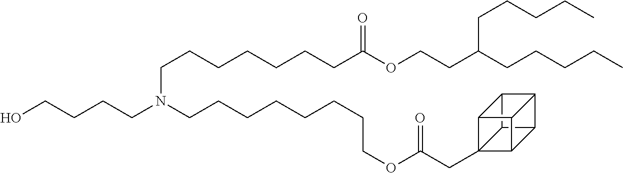 [SMILES: CCCCCC(CCCCC)CCOC(=O)CCCCCCCN(CCCCO)CCCCCCCCOC(=O)CC12C3C4C5C3C1C5C42] |
| C53 | 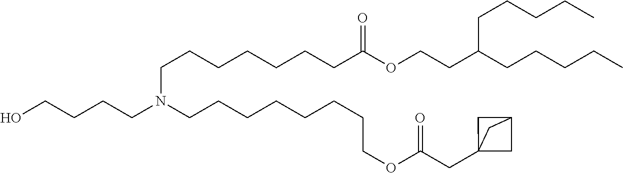 [SMILES: CCCCCC(CCCCC)CCOC(=O)CCCCCCCN(CCCCO)CCCCCCCCOC(=O)CC12CC(C1)C2] |
| C54 | 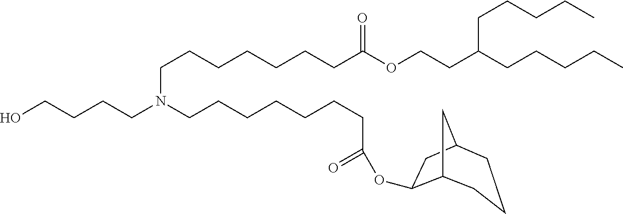 [SMILES: CCCCCC(CCCCC)CCOC(=O)CCCCCCCN(CCCCO)CCCCCCCC(=O)OC1CC2CCCC1C2] |
| C55 | 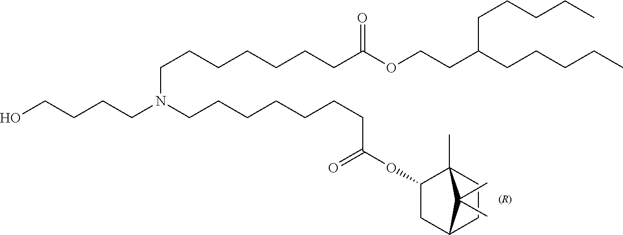 |
| C56 | 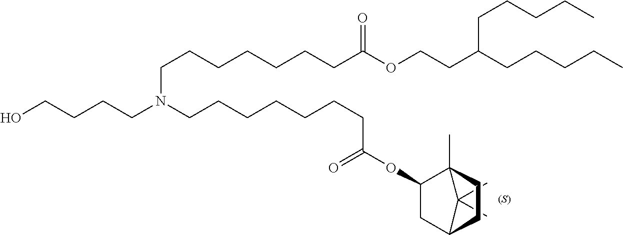 |
| C57 | 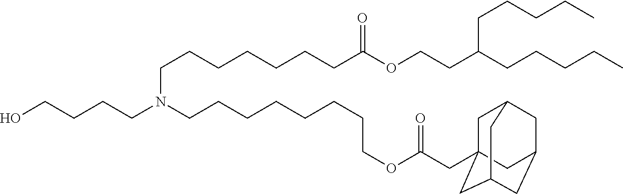 [SMILES: CCCCCC(CCCCC)CCOC(=O)CCCCCCCN(CCCCO)CCCCCCCCOC(=O)CC12CC3CC(CC(C3)C1)C2] |
| C58 | 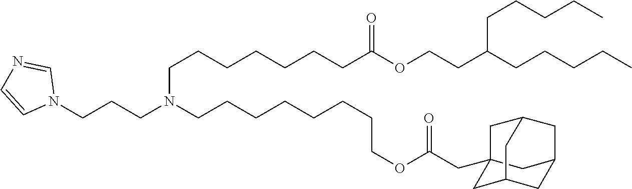 [SMILES: CCCCCC(CCCCC)CCOC(=O)CCCCCCCN(CCCCCCCCOC(=O)CC12CC3CC(CC(C3)C1)C2)CCCn1ccnc1] |
| C59 | 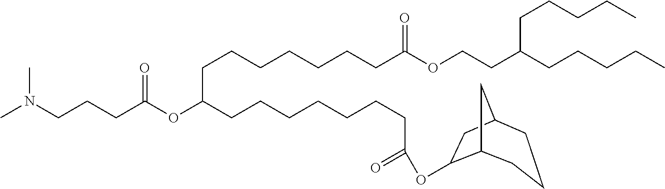 [SMILES: CCCCCC(CCCCC)CCOC(=O)CCCCCCCC(CCCCCCCC(=O)OC1CC2CCCC1C2)OC(=O)CCCN(C)C] |
| C60 | 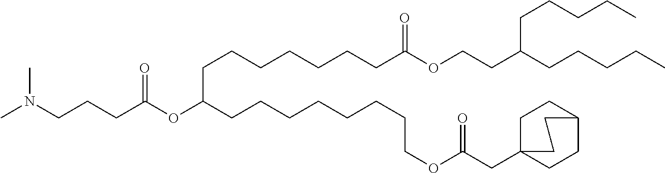 [SMILES: CCCCCC(CCCCC)CCOC(=O)CCCCCCCC(CCCCCCCCOC(=O)CC12CCC(CC1)CC2)OC(=O)CCCN(C)C] |
| C61 | 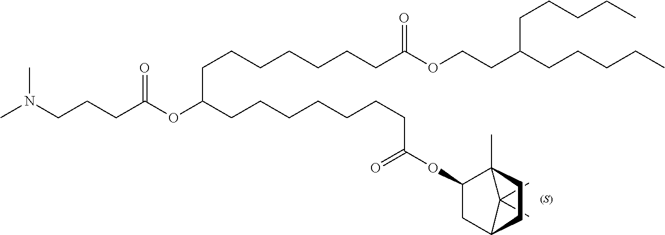 |
| C62 | 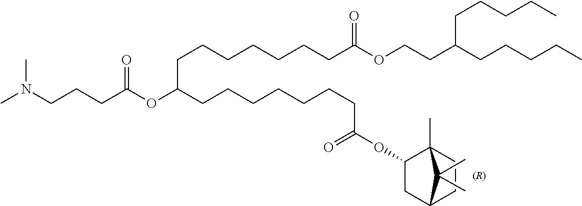 |
| C63 | 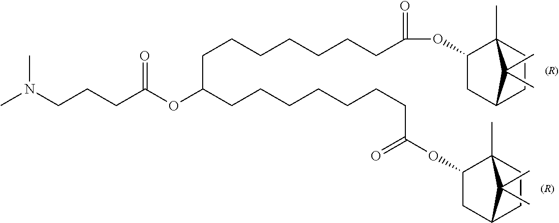 |
| C64 | 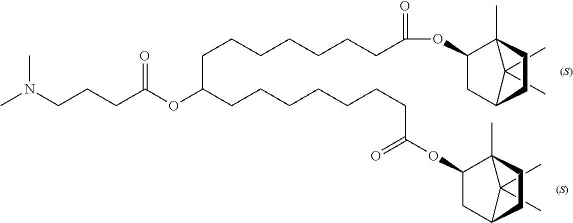 |
| C65 | 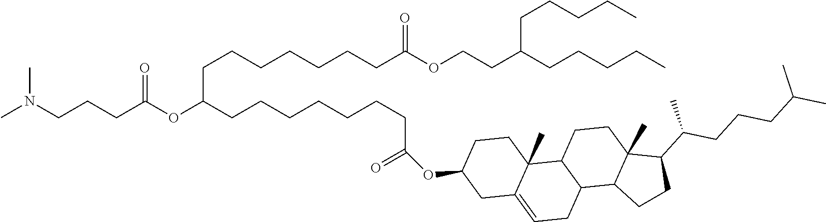 |
| C66 | 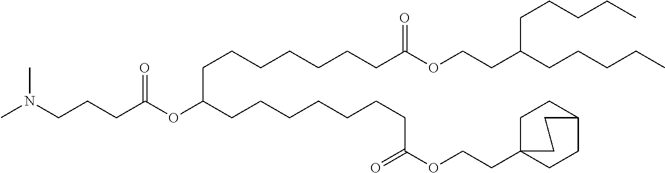 [SMILES: CCCCCC(CCCCC)CCOC(=O)CCCCCCCC(CCCCCCCC(=O)OCCC12CCC(CC1)CC2)OC(=O)CCCN(C)C] |
| C67 | 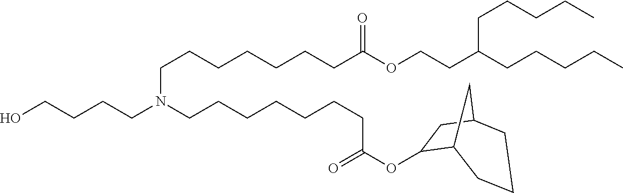 [SMILES: CCCCCC(CCCCC)CCOC(=O)CCCCCCCN(CCCCO)CCCCCCCC(=O)OC1CC2CCCC1C2] |
| C68 | 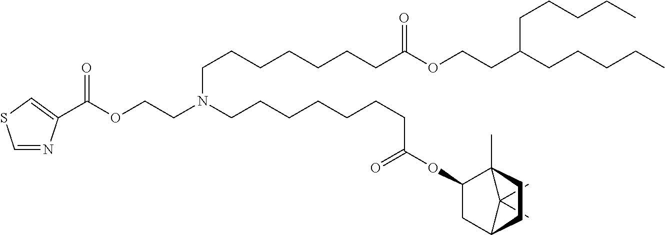 |
| C69 | 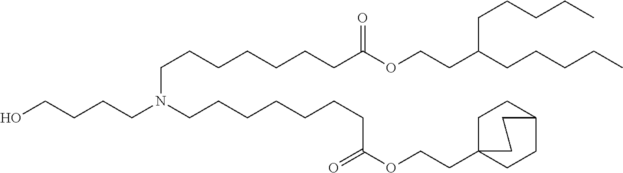 [SMILES: CCCCCC(CCCCC)CCOC(=O)CCCCCCCN(CCCCO)CCCCCCCC(=O)OCCC12CCC(CC1)CC2] |
| C70 | 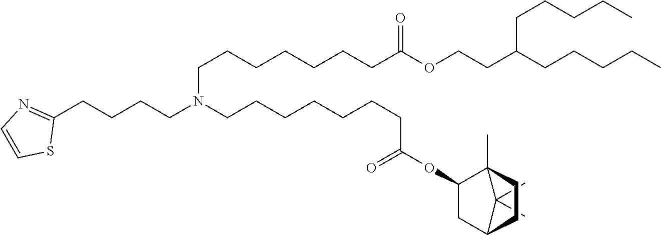 |
| C71 | 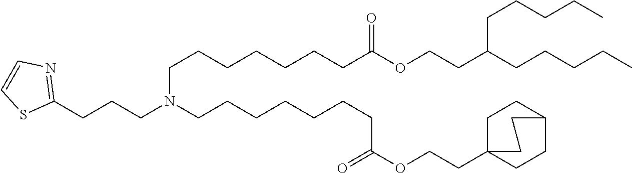 [SMILES: CCCCCC(CCCCC)CCOC(=O)CCCCCCCN(CCCCCCCC(=O)OCCC12CCC(CC1)CC2)CCCc1nccs1] |
| C72 | 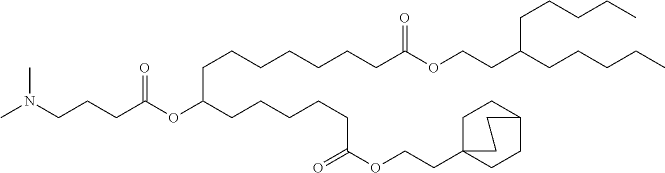 [SMILES: CCCCCC(CCCCC)CCOC(=O)CCCCCCCC(CCCCCC(=O)OCCC12CCC(CC1)CC2)OC(=O)CCCN(C)C] |
| C73 | 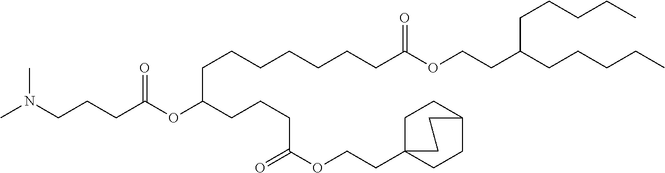 [SMILES: CCCCCC(CCCCC)CCOC(=O)CCCCCCCC(CCCC(=O)OCCC12CCC(CC1)CC2)OC(=O)CCCN(C)C] |
| C74 | 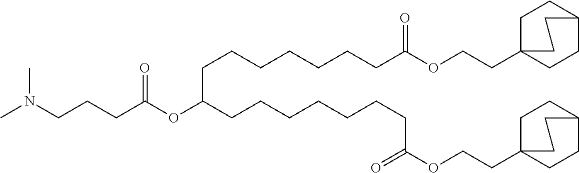 [SMILES: CN(C)CCCC(=O)OC(CCCCCCCC(=O)OCCC12CCC(CC1)CC2)CCCCCCCC(=O)OCCC12CCC(CC1)CC2] |
| C75 | 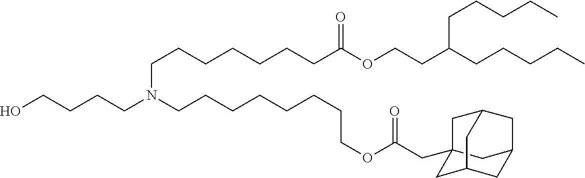 [SMILES: CCCCCC(CCCCC)CCOC(=O)CCCCCCCN(CCCCO)CCCCCCCCOC(=O)CC12CC3CC(CC(C3)C1)C2] |
| C76 | 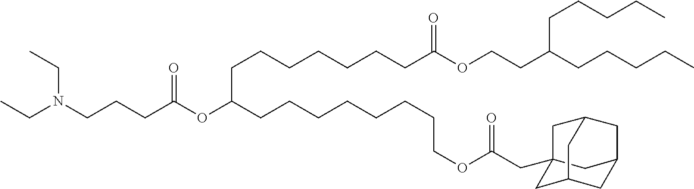 [SMILES: CCCCCC(CCCCC)CCOC(=O)CCCCCCCC(CCCCCCCCOC(=O)CC12CC3CC(CC(C3)C1)C2)OC(=O)CCCN(CC)CC] |
| C77 | 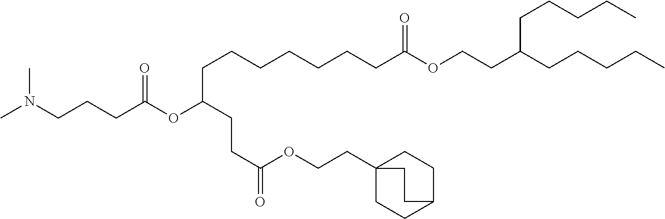 [SMILES: CCCCCC(CCCCC)CCOC(=O)CCCCCCCC(CCC(=O)OCCC12CCC(CC1)CC2)OC(=O)CCCN(C)C] |
| C78 | 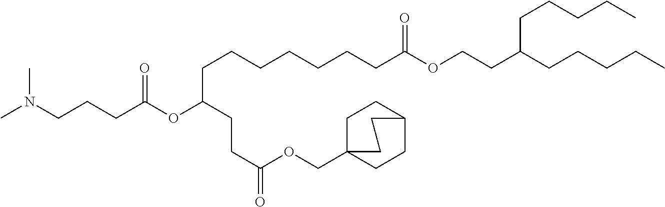 [SMILES: CCCCCC(CCCCC)CCOC(=O)CCCCCCCC(CCC(=O)OCC12CCC(CC1)CC2)OC(=O)CCCN(C)C] |
| C79 | 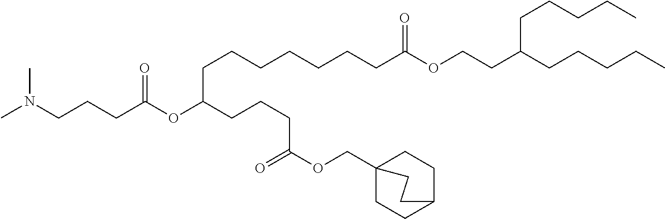 [SMILES: CCCCCC(CCCCC)CCOC(=O)CCCCCCCC(CCCC(=O)OCC12CCC(CC1)CC2)OC(=O)CCCN(C)C] |
| C80 | 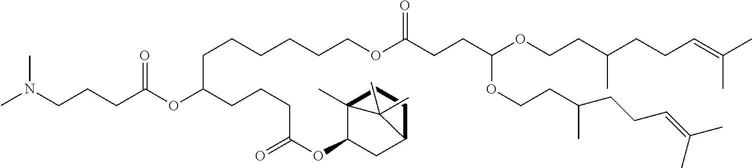 |
| C81 | 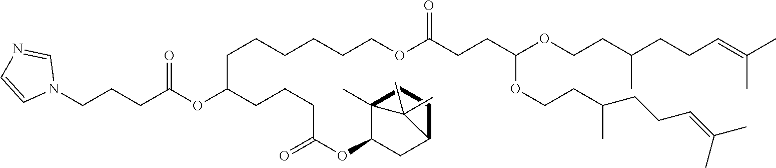 |
| C82 | 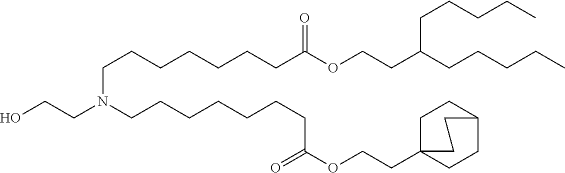 [SMILES: CCCCCC(CCCCC)CCOC(=O)CCCCCCCN(CCO)CCCCCCCC(=O)OCCC12CCC(CC1)CC2] |
| C83 | 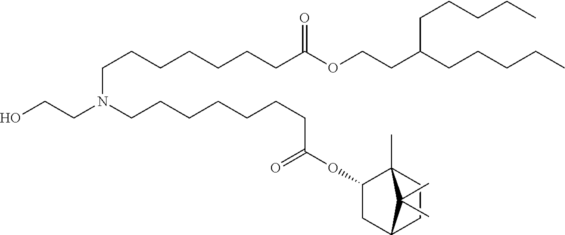 [SMILES: CCCCCC(CCCCC)CCOC(=O)CCCCCCCN(CCO)CCCCCCCC(=O)O[C@H]1C[C@H]2CC[C@]1(C)C2(C)C] |
| C84 | 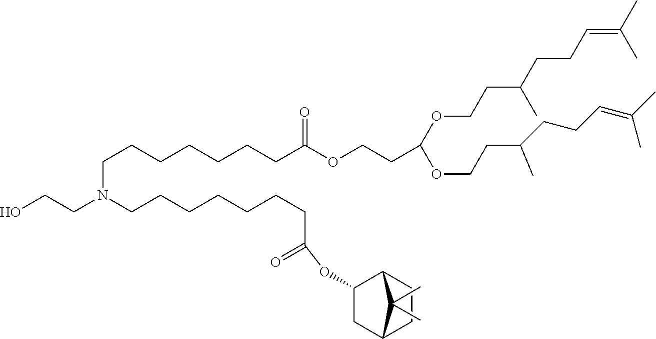 [SMILES: CC(C)=CCCC(C)CCOC(CCOC(=O)CCCCCCCN(CCO)CCCCCCCC(=O)O[C@H]1C[C@H]2CC[C@@H]1C2(C)C)OCCC(C)CCC=C(C)C] |
| C85 | 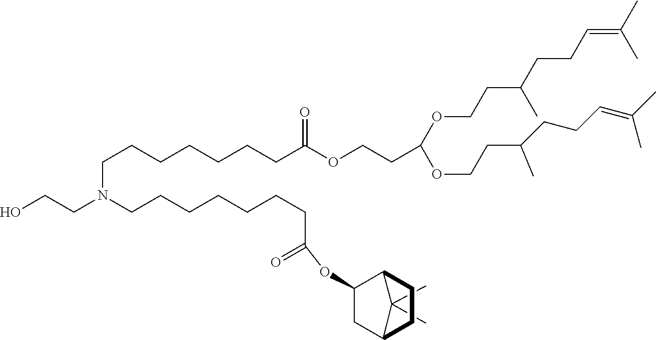 |
| C86 | 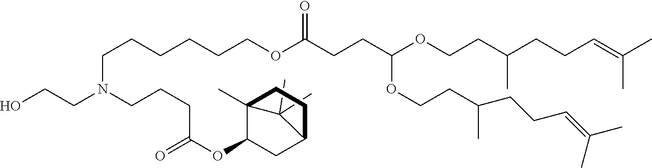 |
| C87 | 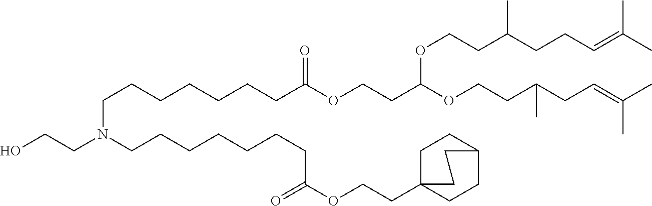 [SMILES: CC(C)=CCCC(C)CCOC(CCOC(=O)CCCCCCCN(CCO)CCCCCCCC(=O)OCCC12CCC(CC1)CC2)OCCC(C)CC=C(C)C] |
| C88 | 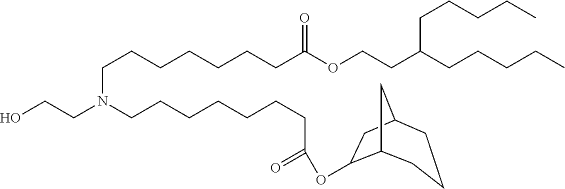 [SMILES: CCCCCC(CCCCC)CCOC(=O)CCCCCCCN(CCO)CCCCCCCC(=O)OC1CC2CCCC1C2] |
| C89 | 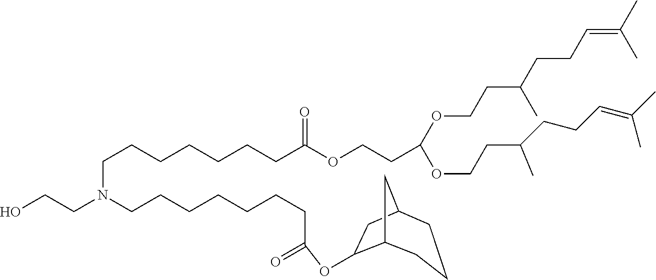 [SMILES: CC(C)=CCCC(C)CCOC(CCOC(=O)CCCCCCCN(CCO)CCCCCCCC(=O)OC1CC2CCCC1C2)OCCC(C)CCC=C(C)C] |
| C90 | 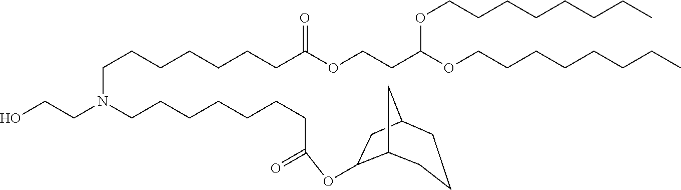 [SMILES: CCCCCCCCOC(CCOC(=O)CCCCCCCN(CCO)CCCCCCCC(=O)OC1CC2CCCC1C2)OCCCCCCCC] |
| C91 | 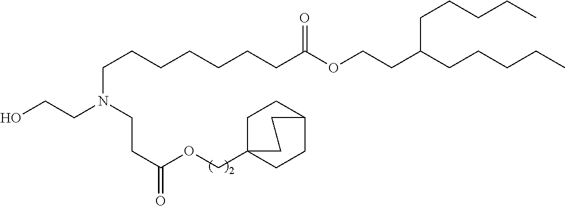 |
| C92 | 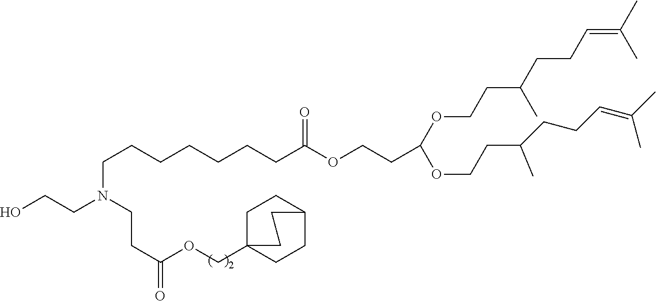 |
| C93 | 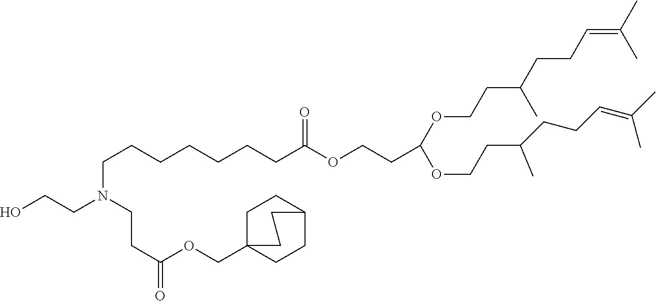 [SMILES: CC(C)=CCCC(C)CCOC(CCOC(=O)CCCCCCCN(CCO)CCC(=O)OCC12CCC(CC1)CC2)OCCC(C)CCC=C(C)C] |

### Series “CX”

In some embodiments, an LNP of the present disclosure comprises an ionizable lipid disclosed in PCT Publication WO2023196931A1, which is incorporated by reference herein, in its entirety.

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, a compound of the present disclosure is represented by Formula (CX-I):

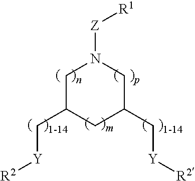

or a pharmaceutically acceptable salt thereof, wherein
Z is selected from the group consisting of a bond,

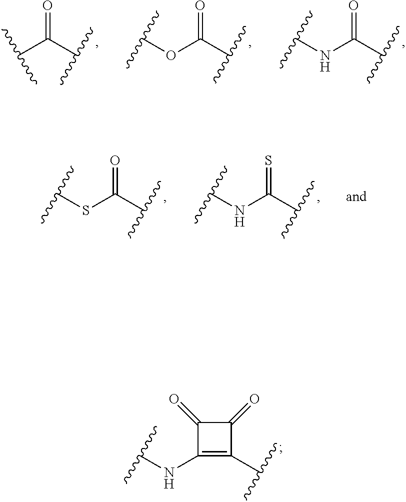

each Y is independently selected from the group consisting of

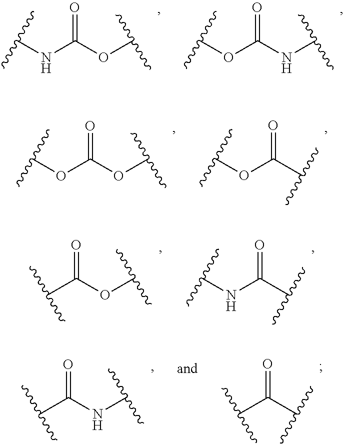

R¹ is —(CH₂)₁₋₆N(R^{a})₂ or —(CH₂)₁₋₆OH;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
R^{2′} is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—; each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-i)

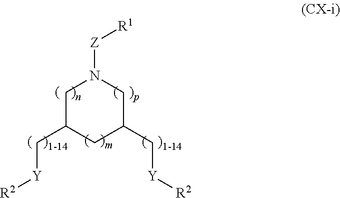

or a pharmaceutically acceptable salt thereof, wherein
Z is selected from the group consisting of a bond,

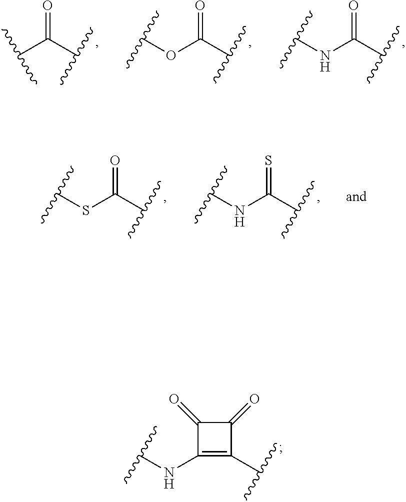

each Y is independently selected from the group consisting of

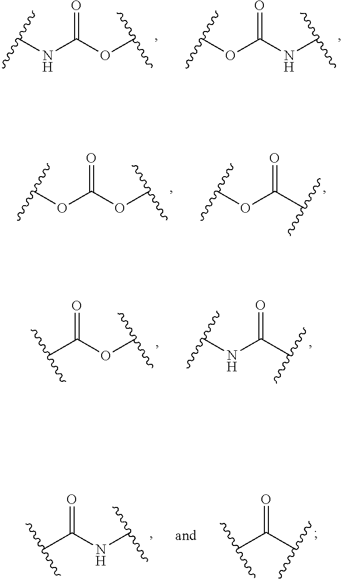

R¹ is —(CH₂)₁₋₆N(R^{a})₂;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, the present disclosure comprises a compound selected from any lipid in Table (IV) below or a pharmaceutically acceptable salt thereof:

TABLE IV
Non-Limiting Examples of Ionizable Lipids

| Structure | # |
|---|---|
| 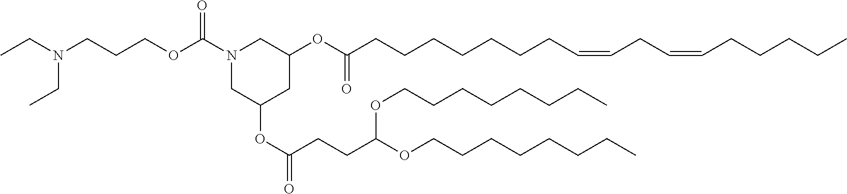 [SMILES: CCCCC/C=C\C/C=C\CCCCCCCC(=O)OC1CC(OC(=O)CCC(OCCCCCCCC)OCCCCCCCC)CN(C(=O)OCCCN(CC)CC)C1] | CX-1 |
| 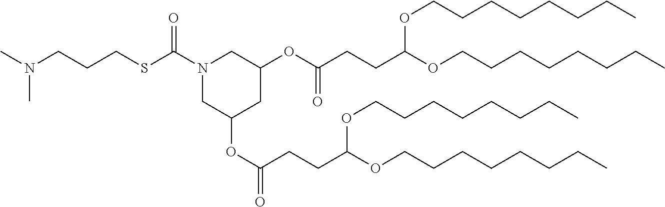 [SMILES: CCCCCCCCOC(CCC(=O)OC1CC(OC(=O)CCC(OCCCCCCCC)OCCCCCCCC)CN(C(=O)SCCCN(C)C)C1)OCCCCCCCC] | CX-2 |
| 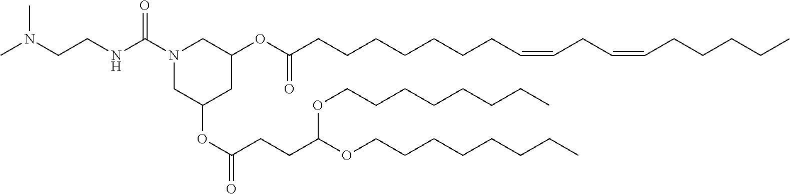 [SMILES: CCCCC/C=C\C/C=C\CCCCCCCC(=O)OC1CC(OC(=O)CCC(OCCCCCCCC)OCCCCCCCC)CN(C(=O)NCCN(C)C)C1] | CX-3 |
| 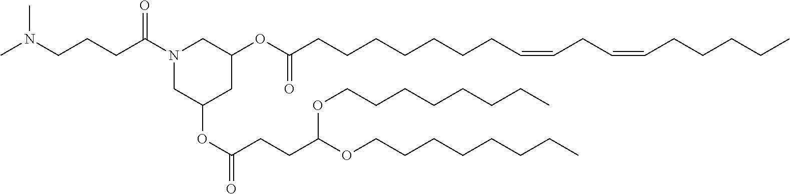 [SMILES: CCCCC/C=C\C/C=C\CCCCCCCC(=O)OC1CC(OC(=O)CCC(OCCCCCCCC)OCCCCCCCC)CN(C(=O)CCCN(C)C)C1] | CX-4 |
| 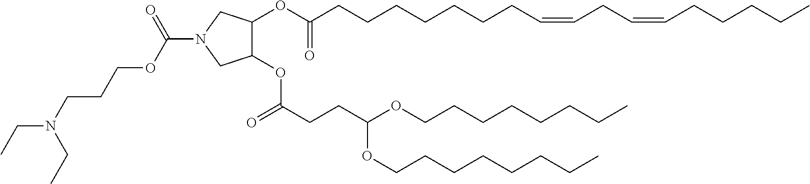 [SMILES: CCCCC/C=C\C/C=C\CCCCCCCC(=O)OC1CN(C(=O)OCCCN(CC)CC)CC1OC(=O)CCC(OCCCCCCCC)OCCCCCCCC] | CX-5 |
| 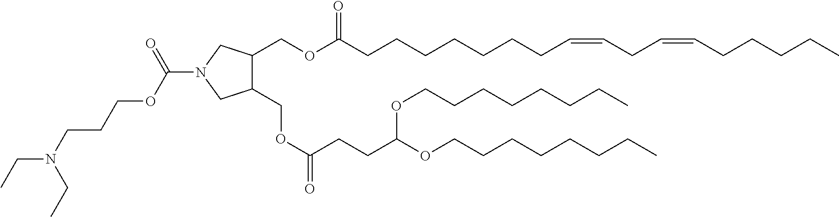 [SMILES: CCCCC/C=C\C/C=C\CCCCCCCC(=O)OCC1CN(C(=O)OCCCN(CC)CC)CC1COC(=O)CCC(OCCCCCCCC)OCCCCCCCC] | CX-6 |
| 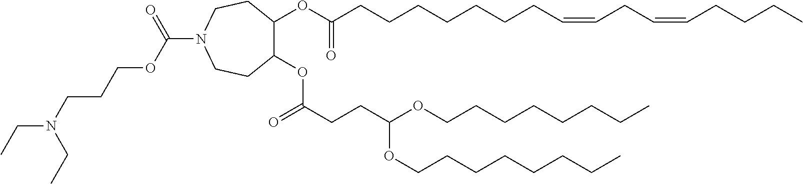 [SMILES: CCCC/C=C\C/C=C\CCCCCCCC(=O)OC1CCN(C(=O)OCCCN(CC)CC)CCC1OC(=O)CCC(OCCCCCCCC)OCCCCCCCC] | CX-7 |
| 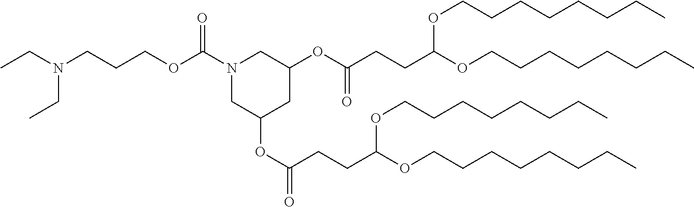 [SMILES: CCCCCCCCOC(CCC(=O)OC1CC(OC(=O)CCC(OCCCCCCCC)OCCCCCCCC)CN(C(=O)OCCCN(CC)CC)C1)OCCCCCCCC] | CX-8 |
| 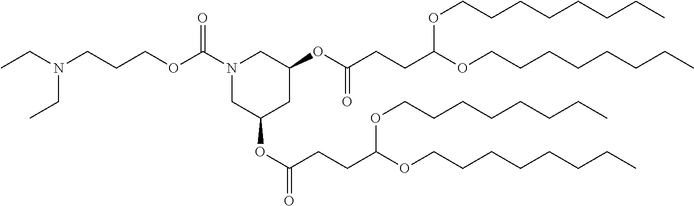 [SMILES: CCCCCCCCOC(CCC(=O)O[C@@H]1C[C@H](OC(=O)CCC(OCCCCCCCC)OCCCCCCCC)CN(C(=O)OCCCN(CC)CC)C1)OCCCCCCCC] | CX-8a |
| 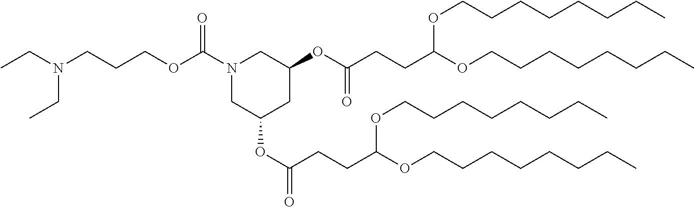 [SMILES: CCCCCCCCOC(CCC(=O)O[C@H]1C[C@H](OC(=O)CCC(OCCCCCCCC)OCCCCCCCC)CN(C(=O)OCCCN(CC)CC)C1)OCCCCCCCC] | CX-8b |
| 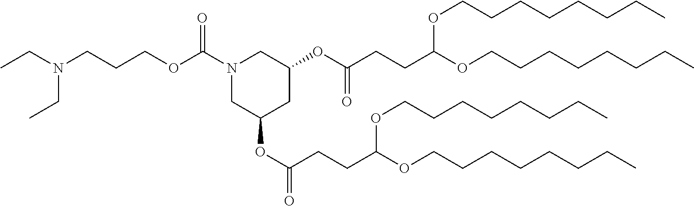 [SMILES: CCCCCCCCOC(CCC(=O)O[C@@H]1C[C@@H](OC(=O)CCC(OCCCCCCCC)OCCCCCCCC)CN(C(=O)OCCCN(CC)CC)C1)OCCCCCCCC] | CX-8c |
| 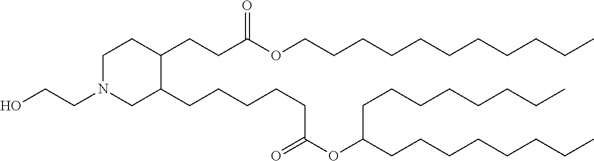 [SMILES: CCCCCCCCCCCOC(=O)CCC1CCN(CCO)CC1CCCCCC(=O)OC(CCCCCCCC)CCCCCCCC] | CX-9 |
| 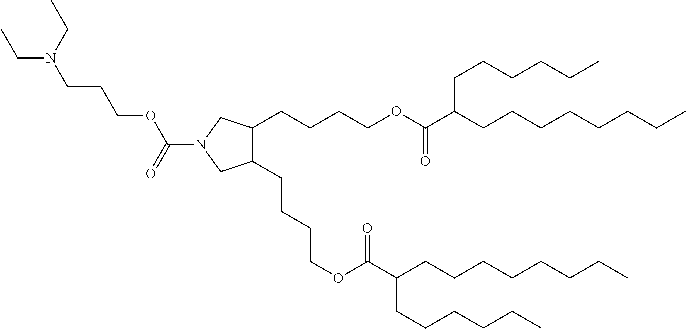 [SMILES: CCCCCCCCC(CCCCCC)C(=O)OCCCCC1CN(C(=O)OCCCN(CC)CC)CC1CCCCOC(=O)C(CCCCCC)CCCCCCCC] | CX-10 |
| 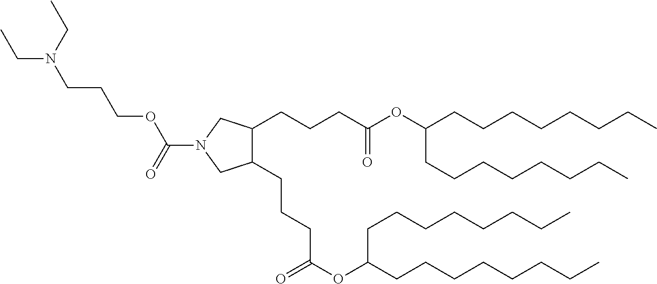 [SMILES: CCCCCCCCC(CCCCCCCC)OC(=O)CCCC1CN(C(=O)OCCCN(CC)CC)CC1CCCC(=O)OC(CCCCCCCC)CCCCCCCC] | CX-11 |
| 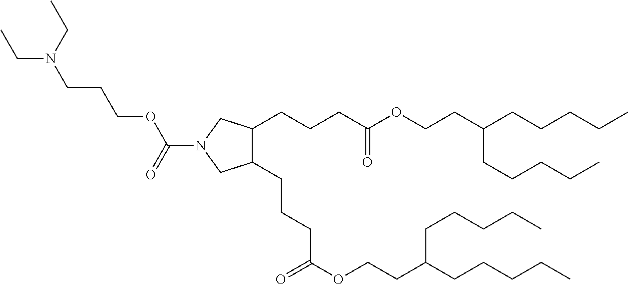 [SMILES: CCCCCC(CCCCC)CCOC(=O)CCCC1CN(C(=O)OCCCN(CC)CC)CC1CCCC(=O)OCCC(CCCCC)CCCCC] | CX-12 |
| 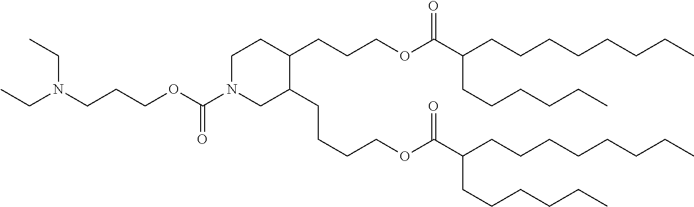 [SMILES: CCCCCCCCC(CCCCCC)C(=O)OCCCCC1CN(C(=O)OCCCN(CC)CC)CCC1CCCOC(=O)C(CCCCCC)CCCCCCCC] | CX-13 |
| 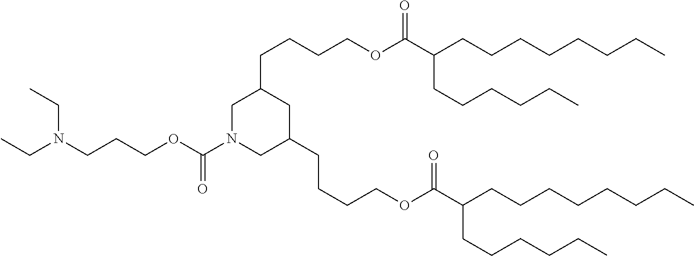 [SMILES: CCCCCCCCC(CCCCCC)C(=O)OCCCCC1CC(CCCCOC(=O)C(CCCCCC)CCCCCCCC)CN(C(=O)OCCCN(CC)CC)C1] | CX-14 |
| 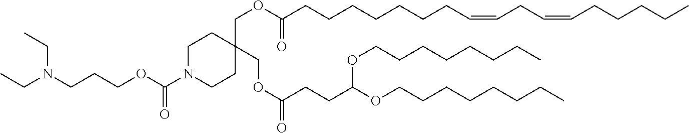 [SMILES: CCCCC/C=C\C/C=C\CCCCCCCC(=O)OCC1(COC(=O)CCC(OCCCCCCCC)OCCCCCCCC)CCN(C(=O)OCCCN(CC)CC)CC1] | CX-15 |
| 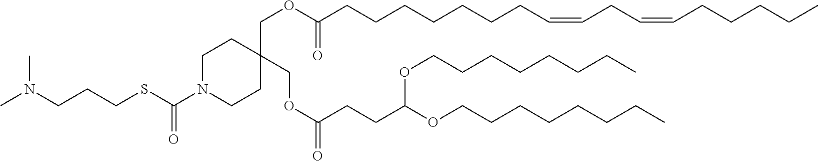 [SMILES: CCCCC/C=C\C/C=C\CCCCCCCC(=O)OCC1(COC(=O)CCC(OCCCCCCCC)OCCCCCCCC)CCN(C(=O)SCCCN(C)C)CC1] | CX-16 |
| 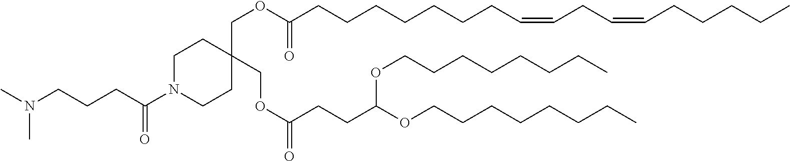 [SMILES: CCCCC/C=C\C/C=C\CCCCCCCC(=O)OCC1(COC(=O)CCC(OCCCCCCCC)OCCCCCCCC)CCN(C(=O)CCCN(C)C)CC1] | CX-17 |
| 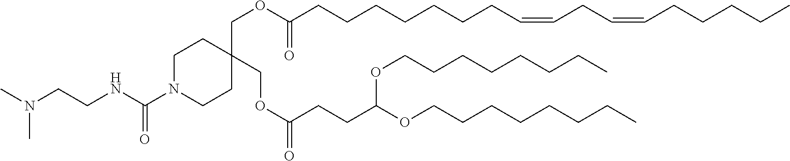 [SMILES: CCCCC/C=C\C/C=C\CCCCCCCC(=O)OCC1(COC(=O)CCC(OCCCCCCCC)OCCCCCCCC)CCN(C(=O)NCCN(C)C)CC1] | CX-18 |
| 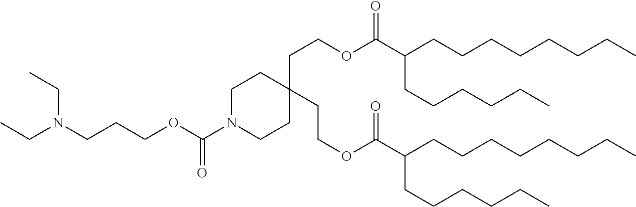 [SMILES: CCCCCCCCC(CCCCCC)C(=O)OCCC1(CCOC(=O)C(CCCCCC)CCCCCCCC)CCN(C(=O)OCCCN(CC)CC)CC1] | CX-19 |
| 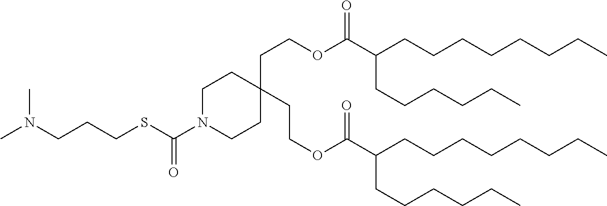 [SMILES: CCCCCCCCC(CCCCCC)C(=O)OCCC1(CCOC(=O)C(CCCCCC)CCCCCCCC)CCN(C(=O)SCCCN(C)C)CC1] | CX-20 |
| 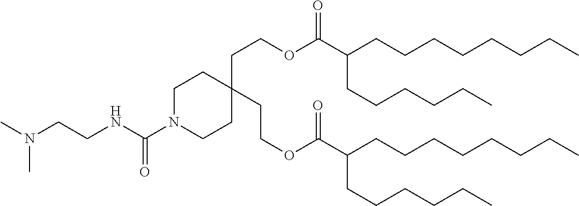 [SMILES: CCCCCCCCC(CCCCCC)C(=O)OCCC1(CCOC(=O)C(CCCCCC)CCCCCCCC)CCN(C(=O)NCCN(C)C)CC1] | CX-21 |
| 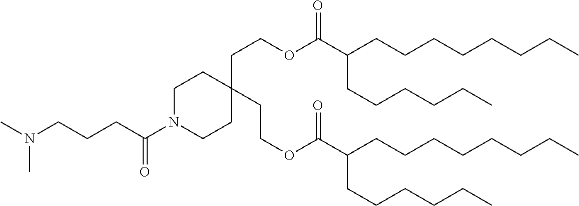 [SMILES: CCCCCCCCC(CCCCCC)C(=O)OCCC1(CCOC(=O)C(CCCCCC)CCCCCCCC)CCN(C(=O)CCCN(C)C)CC1] | CX-22 |
| 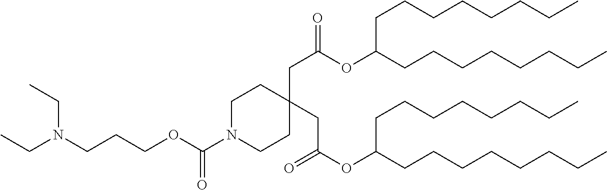 [SMILES: CCCCCCCCC(CCCCCCCC)OC(=O)CC1(CC(=O)OC(CCCCCCCC)CCCCCCCC)CCN(C(=O)OCCCN(CC)CC)CC1] | CX-23 |
| 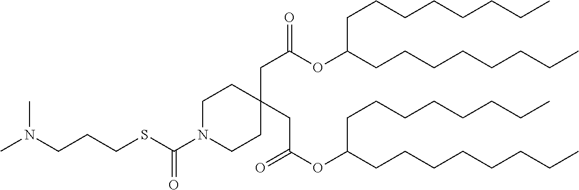 [SMILES: CCCCCCCCC(CCCCCCCC)OC(=O)CC1(CC(=O)OC(CCCCCCCC)CCCCCCCC)CCN(C(=O)SCCCN(C)C)CC1] | CX-24 |
| 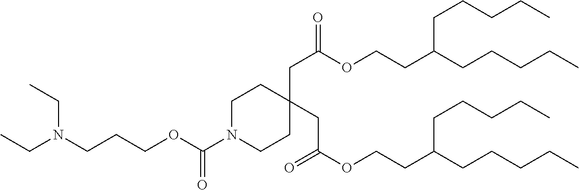 [SMILES: CCCCCC(CCCCC)CCOC(=O)CC1(CC(=O)OCCC(CCCCC)CCCCC)CCN(C(=O)OCCCN(CC)CC)CC1] | CX-25 |
| 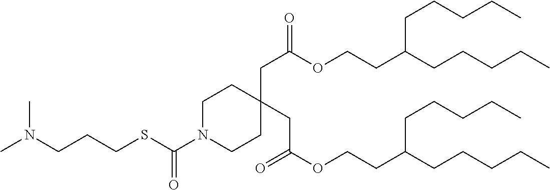 [SMILES: CCCCCC(CCCCC)CCOC(=O)CC1(CC(=O)OCCC(CCCCC)CCCCC)CCN(C(=O)SCCCN(C)C)CC1] | CX-26 |
| 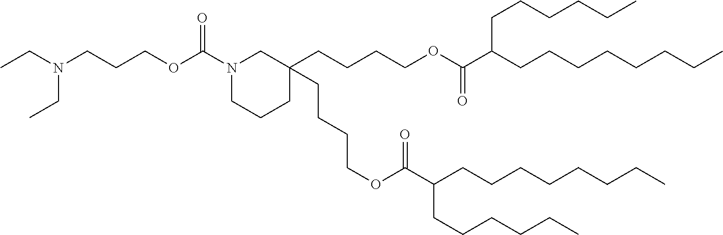 [SMILES: CCCCCCCCC(CCCCCC)C(=O)OCCCCC1(CCCCOC(=O)C(CCCCCC)CCCCCCCC)CCCN(C(=O)OCCCN(CC)CC)C1] | CX-27 |
| 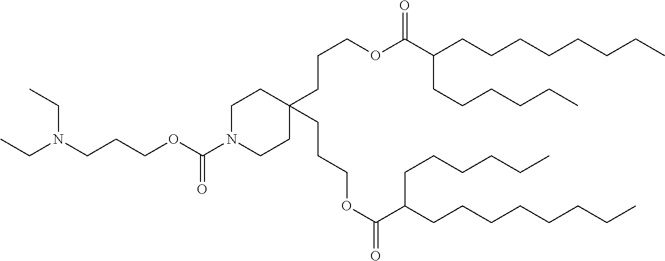 [SMILES: CCCCCCCCC(CCCCCC)C(=O)OCCCC1(CCCOC(=O)C(CCCCCC)CCCCCCCC)CCN(C(=O)OCCCN(CC)CC)CC1] | CX-28 |
| 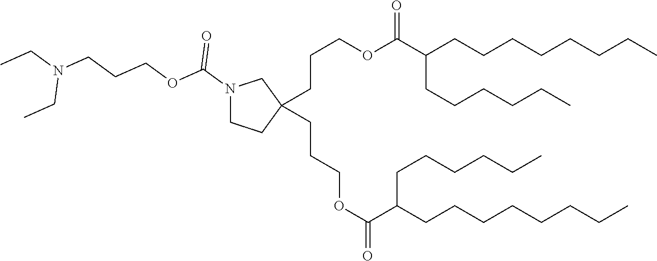 [SMILES: CCCCCCCCC(CCCCCC)C(=O)OCCCC1(CCCOC(=O)C(CCCCCC)CCCCCCCC)CCN(C(=O)OCCCN(CC)CC)C1] | CX-29 |
| 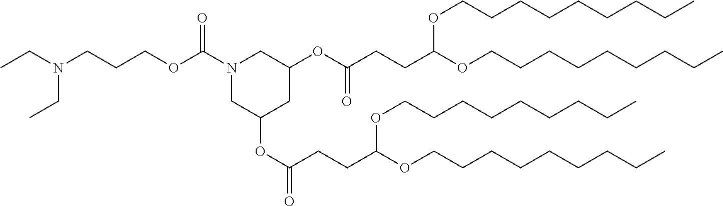 [SMILES: CCCCCCCCCOC(CCC(=O)OC1CC(OC(=O)CCC(OCCCCCCCCC)OCCCCCCCCC)CN(C(=O)OCCCN(CC)CC)C1)OCCCCCCCCC] | CX-30 |
| 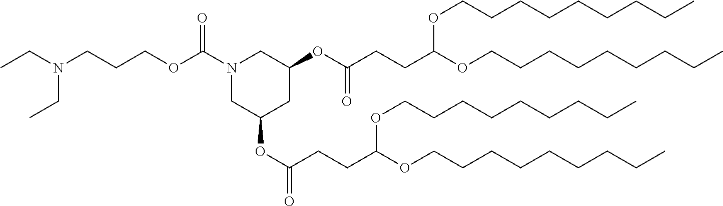 [SMILES: CCCCCCCCCOC(CCC(=O)O[C@@H]1C[C@H](OC(=O)CCC(OCCCCCCCCC)OCCCCCCCCC)CN(C(=O)OCCCN(CC)CC)C1)OCCCCCCCCC] | CX-30a |
| 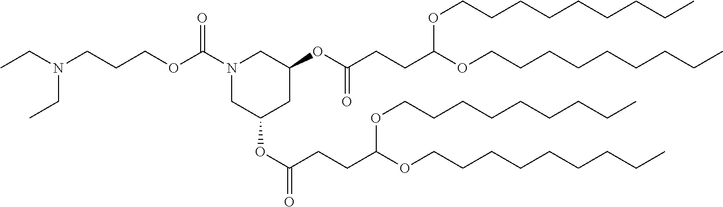 [SMILES: CCCCCCCCCOC(CCC(=O)O[C@H]1C[C@H](OC(=O)CCC(OCCCCCCCCC)OCCCCCCCCC)CN(C(=O)OCCCN(CC)CC)C1)OCCCCCCCCC] | CX-30b |
| 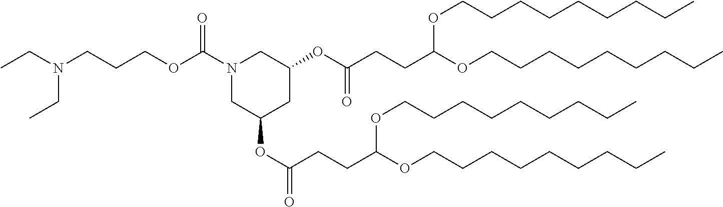 [SMILES: CCCCCCCCCOC(CCC(=O)O[C@@H]1C[C@@H](OC(=O)CCC(OCCCCCCCCC)OCCCCCCCCC)CN(C(=O)OCCCN(CC)CC)C1)OCCCCCCCCC] | CX-30c |

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

### Series “CZ”

In some embodiments, an LNP of the present disclosure comprises an ionizable lipid disclosed in PCT Publication WO2023196931A1, which is incorporated by reference herein, in its entirety.

In some embodiments, a compound of the present disclosure is represented by Formula (CZ-I)

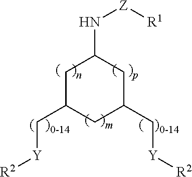

or a pharmaceutically acceptable salt thereof, wherein
Z is selected from the group consisting of a bond,

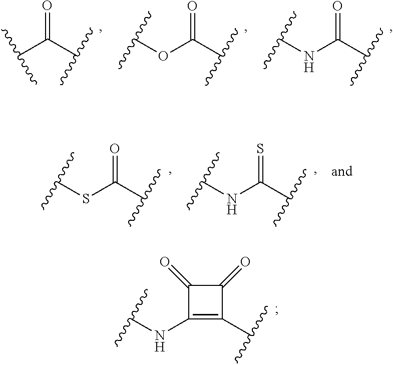

each Y is independently selected from the group consisting of,

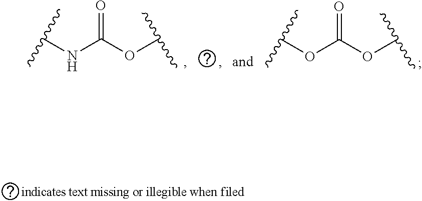 [SMILES: *NC(=O)O* ; *OC(=O)O*]

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, the present disclosure comprises a compound selected from any lipid in Table (V) below or a pharmaceutically acceptable salt thereof:

TABLE V
Non-Limiting Examples of Ionizable Lipids

| Structure | # |
|---|---|
| 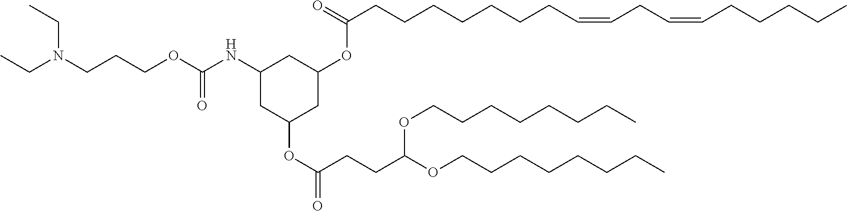 [SMILES: CCCCC/C=C\C/C=C\CCCCCCCC(=O)OC1CC(NC(=O)OCCCN(CC)CC)CC(OC(=O)CCC(OCCCCCCCC)OCCCCCCCC)C1] | CZ-1 |
| 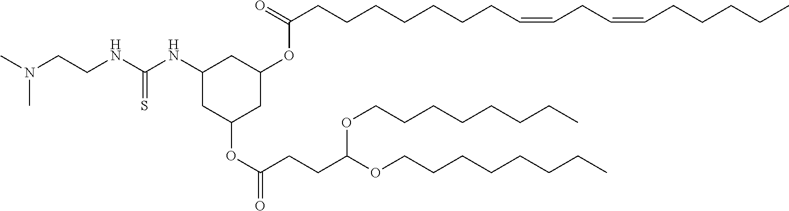 [SMILES: CCCCC/C=C\C/C=C\CCCCCCCC(=O)OC1CC(NC(=S)NCCN(C)C)CC(OC(=O)CCC(OCCCCCCCC)OCCCCCCCC)C1] | CZ-2 |
| 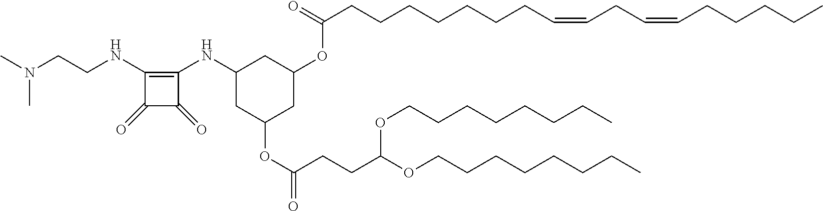 [SMILES: CCCCC/C=C\C/C=C\CCCCCCCC(=O)OC1CC(Nc2c(NCCN(C)C)c(=O)c2=O)CC(OC(=O)CCC(OCCCCCCCC)OCCCCCCCC)C1] | CZ-3 |
| 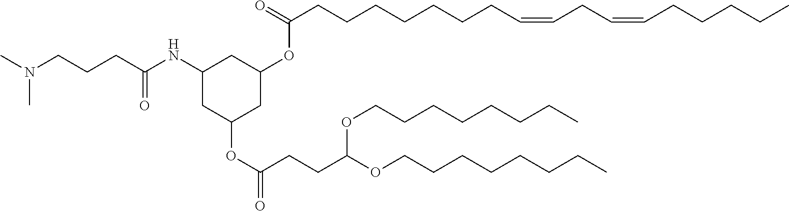 [SMILES: CCCCC/C=C\C/C=C\CCCCCCCC(=O)OC1CC(NC(=O)CCCN(C)C)CC(OC(=O)CCC(OCCCCCCCC)OCCCCCCCC)C1] | CZ-4 |
| 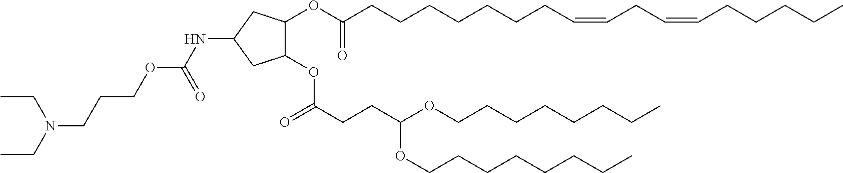 [SMILES: CCCCC/C=C\C/C=C\CCCCCCCC(=O)OC1CC(NC(=O)OCCCN(CC)CC)CC1OC(=O)CCC(OCCCCCCCC)OCCCCCCCC] | CZ-5 |
| 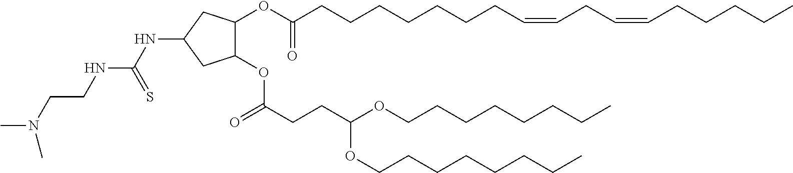 [SMILES: CCCCC/C=C\C/C=C\CCCCCCCC(=O)OC1CC(NC(=S)NCCN(C)C)CC1OC(=O)CCC(OCCCCCCCC)OCCCCCCCC] | CZ-6 |
| 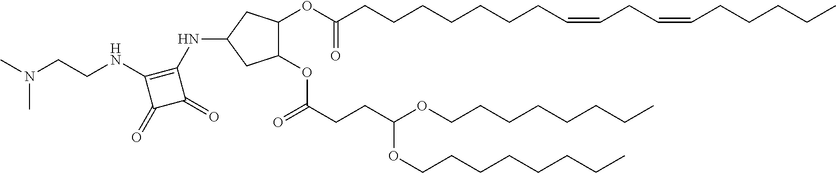 [SMILES: CCCCC/C=C\C/C=C\CCCCCCCC(=O)OC1CC(Nc2c(NCCN(C)C)c(=O)c2=O)CC1OC(=O)CCC(OCCCCCCCC)OCCCCCCCC] | CZ-7 |
| 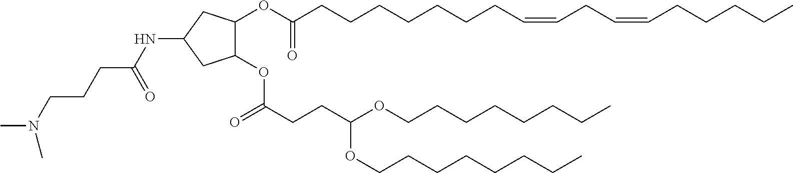 [SMILES: CCCCC/C=C\C/C=C\CCCCCCCC(=O)OC1CC(NC(=O)CCCN(C)C)CC1OC(=O)CCC(OCCCCCCCC)OCCCCCCCC] | CZ-8 |
| 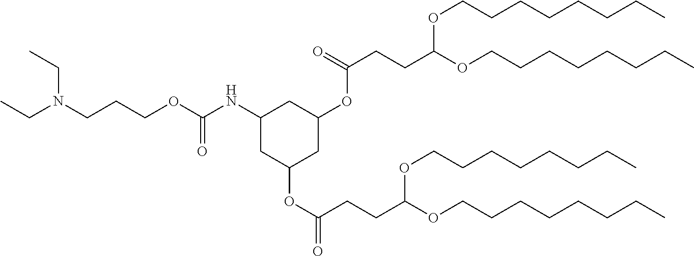 [SMILES: CCCCCCCCOC(CCC(=O)OC1CC(NC(=O)OCCCN(CC)CC)CC(OC(=O)CCC(OCCCCCCCC)OCCCCCCCC)C1)OCCCCCCCC] | CZ-9 |
| 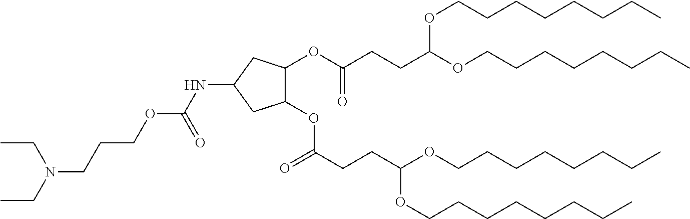 [SMILES: CCCCCCCCOC(CCC(=O)OC1CC(NC(=O)OCCCN(CC)CC)CC1OC(=O)CCC(OCCCCCCCC)OCCCCCCCC)OCCCCCCCC] | CZ-10 |
| 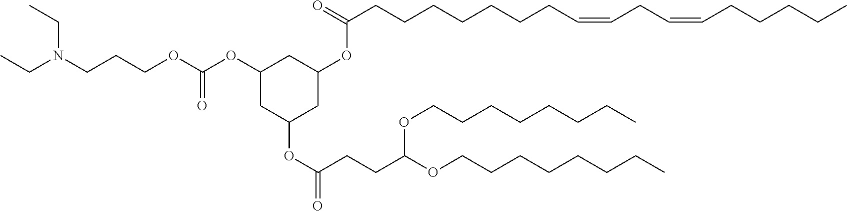 [SMILES: CCCCC/C=C\C/C=C\CCCCCCCC(=O)OC1CC(OC(=O)CCC(OCCCCCCCC)OCCCCCCCC)CC(OC(=O)OCCCN(CC)CC)C1] | CZ-11 |
| 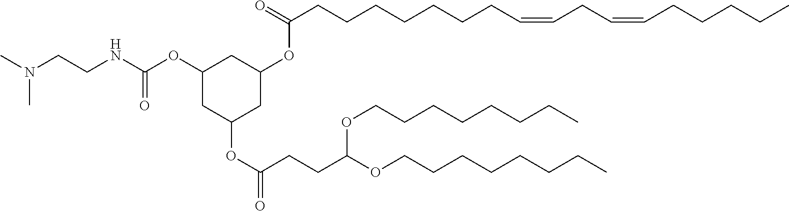 [SMILES: CCCCC/C=C\C/C=C\CCCCCCCC(=O)OC1CC(OC(=O)CCC(OCCCCCCCC)OCCCCCCCC)CC(OC(=O)NCCN(C)C)C1] | CZ-12 |
| 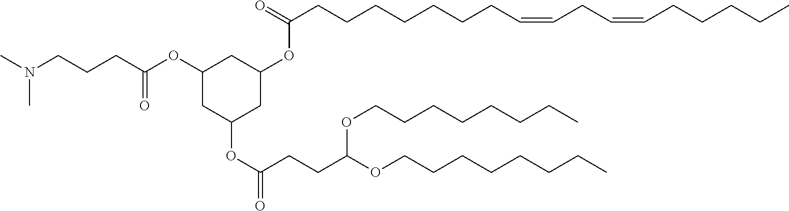 [SMILES: CCCCC/C=C\C/C=C\CCCCCCCC(=O)OC1CC(OC(=O)CCCN(C)C)CC(OC(=O)CCC(OCCCCCCCC)OCCCCCCCC)C1] | CZ-13 |
| 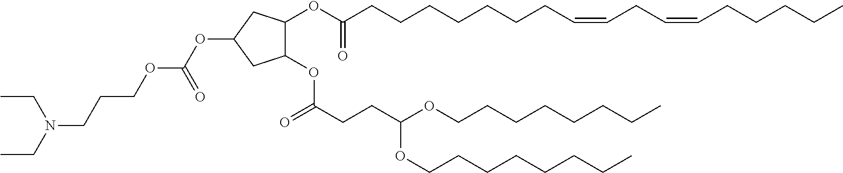 [SMILES: CCCCC/C=C\C/C=C\CCCCCCCC(=O)OC1CC(OC(=O)OCCCN(CC)CC)CC1OC(=O)CCC(OCCCCCCCC)OCCCCCCCC] | CZ-14 |
| 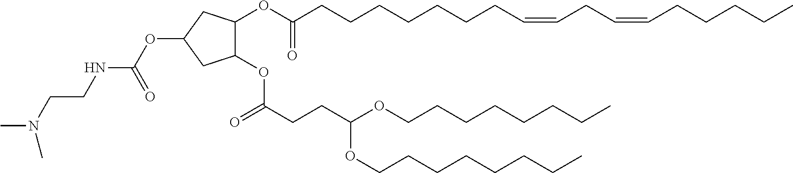 [SMILES: CCCCC/C=C\C/C=C\CCCCCCCC(=O)OC1CC(OC(=O)NCCN(C)C)CC1OC(=O)CCC(OCCCCCCCC)OCCCCCCCC] | CZ-15 |
| 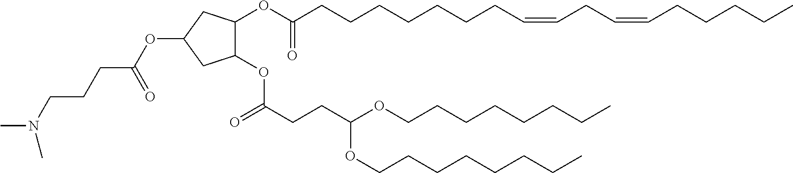 [SMILES: CCCCC/C=C\C/C=C\CCCCCCCC(=O)OC1CC(OC(=O)CCCN(C)C)CC1OC(=O)CCC(OCCCCCCCC)OCCCCCCCC] | CZ-16 |
| 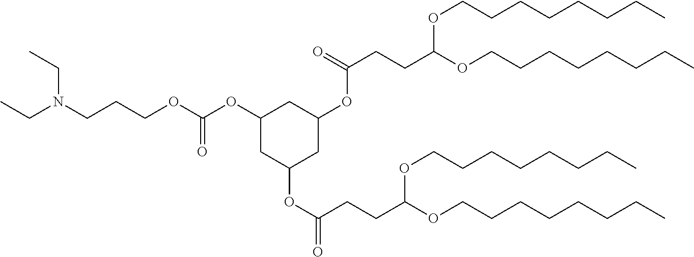 [SMILES: CCCCCCCCOC(CCC(=O)OC1CC(OC(=O)CCC(OCCCCCCCC)OCCCCCCCC)CC(OC(=O)OCCCN(CC)CC)C1)OCCCCCCCC] | CZ-17 |
| 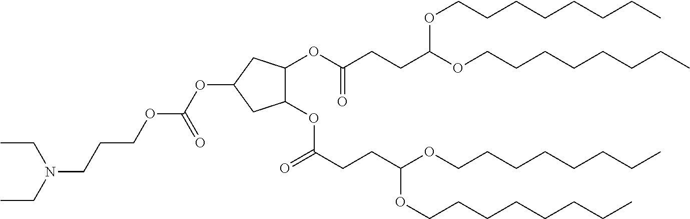 [SMILES: CCCCCCCCOC(CCC(=O)OC1CC(OC(=O)OCCCN(CC)CC)CC1OC(=O)CCC(OCCCCCCCC)OCCCCCCCC)OCCCCCCCC] | CZ-18 |

### Series “S”

In some embodiments, an LNP of the present disclosure comprises an ionizable lipid disclosed in PCT Application PCT/US2024/019990, which is incorporated by reference herein, in its entirety.

In some embodiments, ionizable lipids of the present disclosure have a structure of Formula (S-I):

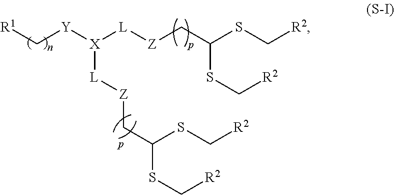

or a pharmaceutically acceptable salt thereof, wherein:
X is N or CH;
Y is a bond,

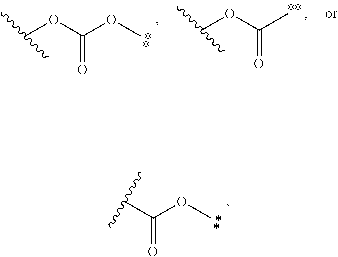

wherein bond marked with an “**” is attached to X;
each Z is independently selected from the group consisting of:

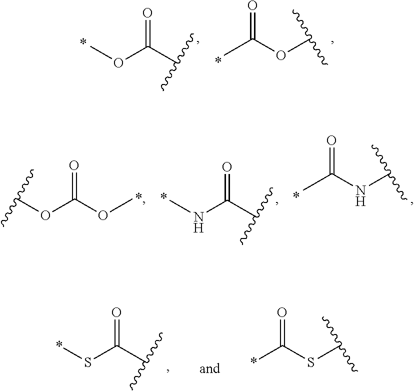

wherein the bond marked with an “*” is attached to L;
each L is independently C₂-C₁₀ alkylenyl;

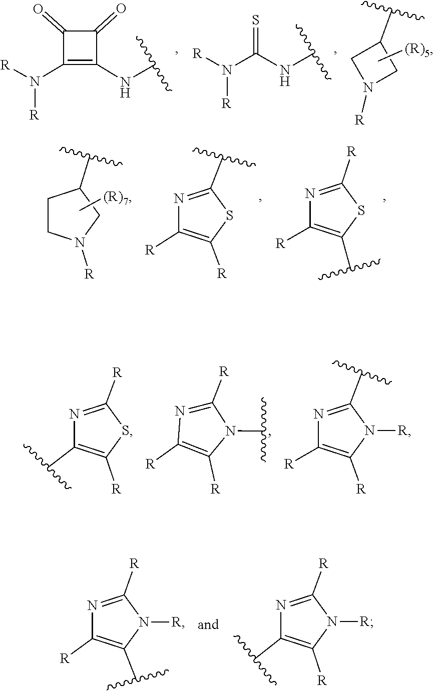

R¹ is OH, N(R³) 2
each R is independently —H or C₁-C₆ aliphatic;
each R² is independently selected from optionally substituted C₂₋₁₄alkyl and C₂₋₁₄alkenyl, wherein any —(CH₂)₂— of the C₂-C₁₄ alkyl can be optionally replaced with C₃-C₆ cycloalkylenyl;
each R³ independently selected from is H and C₁₋₆ alkyl;
n is selected from 1 to 6; and
each p is independently selected from 1 to 6.

### X

In some embodiments, ionizable lipids of the present disclosure have a structure of Formula (S-I), wherein X is N. In some embodiments, ionizable lipids of the present disclosure have a structure of Formula (S-I), wherein X is CH.

### Y

In some embodiments, ionizable lipids of the present disclosure have a structure of Formula (S-I), wherein Y is a bond. In some embodiments, ionizable lipids of the present disclosure have a structure of Formula (S-I), wherein Y is

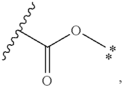 [SMILES: *C(=O)OC]

wherein bond marked with an “**” is attached to X. In some embodiments, ionizable lipids of the present disclosure have a structure of Formula (S-I), wherein Y is

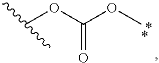 [SMILES: *OC(=O)OC]

wherein bond marked with an “**” is attached to X. In some embodiments, ionizable lipids of the present disclosure have a structure of Formula (S-I), wherein Y is

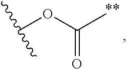 [SMILES: *OC(C)=O]

wherein bond marked with an “**” is attached to X.

### Z

In some embodiments, ionizable lipids of the present disclosure have a structure of Formula (S-I), wherein Z is

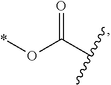 [SMILES: *OC(*)=O]

wherein bond marked with an “*” is attached to X. In some embodiments, ionizable lipids of the present disclosure have a structure of Formula (S-I), wherein Z is

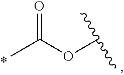 [SMILES: *OC(C)=O]

wherein bond marked with an “*” is attached to X. In some embodiments, ionizable lipids of the present disclosure have a structure of Formula (S-I), wherein Z is

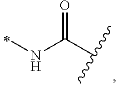

wherein bond marked with an “*” is attached to X. In some embodiments, ionizable lipids of the present disclosure have a structure of Formula (S-I), wherein Z is

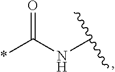 [SMILES: *NC(*)=O]

wherein bond marked with an “*” is attached to X. In some embodiments, ionizable lipids of the present disclosure have a structure of Formula (S-I), wherein Z is

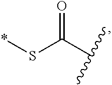 [SMILES: *SC(*)=O]

wherein bond marked with an “*” is attached to X. In some embodiments, ionizable lipids of the present disclosure have a structure of Formula (S-I), wherein Z is

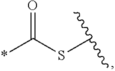 [SMILES: *SC(*)=O]

wherein bond marked with an “*” is attached to X. In some embodiments, ionizable lipids of the present disclosure have a structure of Formula (S-I), wherein Z is

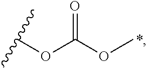

wherein bond marked with an “*” is attached to X.

### L

In some embodiments, ionizable lipids of the present disclosure have a structure of Formula (S-I), wherein L is C₂-C₁₀ alkylenyl. In some embodiments, ionizable lipids of the present disclosure have a structure of Formula (S-I), wherein L is C₅-C₈ alkylenyl. In some embodiments, ionizable lipids of the present disclosure have a structure of Formula (S-I), wherein L is C₅ alkylenyl. In some embodiments, ionizable lipids of the present disclosure have a structure of Formula (S-I), wherein L is C₆ alkylenyl. In some embodiments, ionizable lipids of the present disclosure have a structure of Formula (S-I), wherein L is C₇ alkylenyl. In some embodiments, ionizable lipids of the present disclosure have a structure of Formula (S-I), wherein L is C₈ alkylenyl.

### R¹

In some embodiments, ionizable lipids of the present disclosure have a structure of Formula (S-I), wherein R¹ is OH. In some embodiments, ionizable lipids of the present disclosure have a structure of Formula (S-I), wherein R¹ is N(R³)₂. In some embodiments, ionizable lipids of the present disclosure have a structure of Formula (S-I), wherein R¹ is

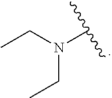 [SMILES: *N(CC)CC]

In some embodiments, ionizable lipids of the present disclosure have a structure of Formula (S-I), wherein R¹ is

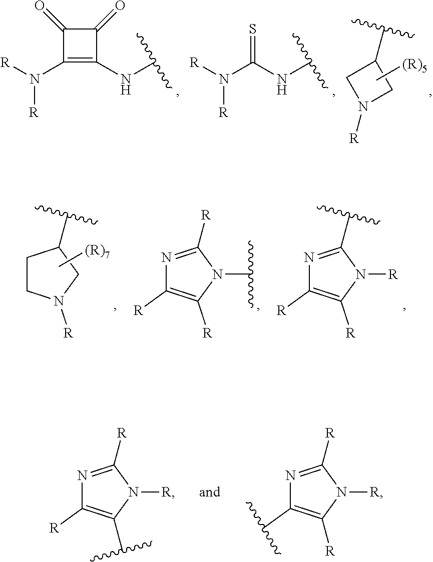

wherein each R is independently —H or C₁-C₆ aliphatic. In certain embodiments, R¹ is

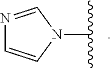 [SMILES: *n1ccnc1]

In some embodiments, ionizable lipids of the present disclosure have a structure of Formula (S-Ia):

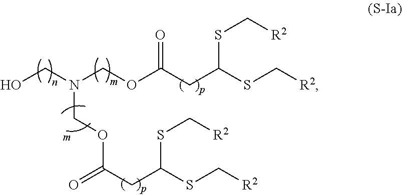

or a pharmaceutically acceptable salt thereof, wherein:
each R² is independently selected from optionally substituted C₂₋₁₄alkyl and C₂₋₁₄alkenyl, wherein any —(CH₂)₂— of the C₂-C₁₄ alkyl can be optionally replaced with C₃-C₆ cycloalkylenyl;
n is selected from 1 to 4;
each m is independently selected from 2 to 10; and
each p is independently selected from 2 to 6.

In some embodiments, ionizable lipids of the present disclosure have a structure of Formula (S-Ib):

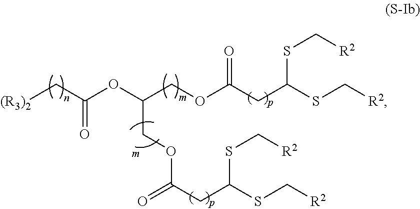

or a pharmaceutically acceptable salt thereof, wherein:
each R² is independently selected from optionally substituted C₂₋₁₄alkyl and C₂₋₁₄alkenyl, wherein any —(CH₂)₂— of the C₂-C₁₄ alkyl can be optionally replaced with C₃-C₆ cycloalkylenyl;
each R³ independently selected from is H and C₁₋₆alkylene;
n is selected from 1 to 4;
each m is independently selected from 2 to 10; and
each p is independently selected from 2 to 6.

R²

In some embodiments, ionizable lipids of the present disclosure have a structure of Formula (S-I), Formula (S-Ia), or Formula (S-Ib), wherein R² is optionally substituted C₂₋₁₄alkyl. In some embodiments, ionizable lipids of the present disclosure have a structure of Formula (S-I), Formula (S-Ia), or Formula (S-Ib), wherein R² is optionally substituted C₇-12alkyl. In some embodiments, ionizable lipids of the present disclosure have a structure of Formula (S-I), Formula (S-Ia), or Formula (S-Ib), wherein R² is independently selected from the group consisting of:

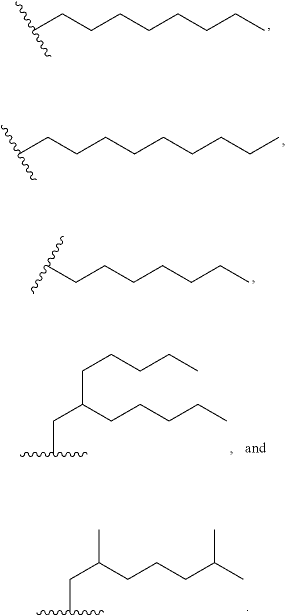

In some embodiments, ionizable lipids of the present disclosure have a structure of Formula (S-I), Formula (S-Ia), or Formula (S-Ib), wherein R² is

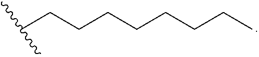 [SMILES: *CCCCCCCC]

In some embodiments, ionizable lipids of the present disclosure have a structure of Formula (S-I), Formula (S-Ia), or Formula (S-Ib), wherein R² is

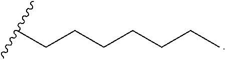 [SMILES: *CCCCCCC]

In some embodiments, ionizable lipids of the present disclosure have a structure of Formula (S-I), Formula (S-Ia), or Formula (S-Ib), wherein

### R² is

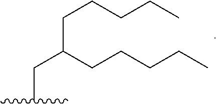 [SMILES: *CC(CCCCC)CCCCC ; "."]

In some embodiments, ionizable lipids of the present disclosure have a structure of Formula (S-I), Formula (S-Ia), or Formula (S-Ib), wherein R² is

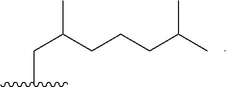 [SMILES: *CC(C)CCCC(C)C]

In some embodiments, ionizable lipids of the present disclosure have a structure of Formula (S-I), Formula (S-Ia), or Formula (S-Ib), wherein R² is optionally substituted C₂₋₁₄alkenyl. In some embodiments, ionizable lipids of the present disclosure have a structure of Formula (S-I), Formula (S-Ia), or Formula (S-Ib), wherein R² is independently selected from:

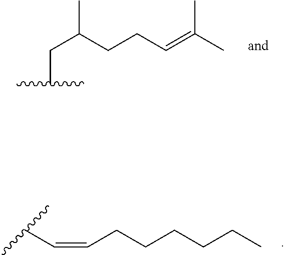

In some embodiments, ionizable lipids of the present disclosure have a structure of Formula (S-I), Formula (S-Ia), or Formula (S-Ib), wherein R² is

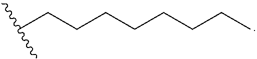 [SMILES: *CCCCCCCC]

In some embodiments, ionizable lipids of the present disclosure have a structure of Formula (S-I), Formula (S-Ia), or Formula (S-Ib), wherein R² is optionally substituted C₈-9alkenyl. In some embodiments, ionizable lipids of the present disclosure have a structure of Formula (S-I), Formula (S-Ia), or Formula (S-Ib), wherein R² is

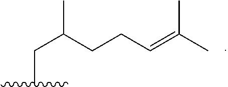 [SMILES: *CC(C)CCC=C(C)C]

In some embodiments, ionizable lipids of the present disclosure have a structure of Formula (S-I), Formula (S-Ia), or Formula (S-Ib), wherein R² is

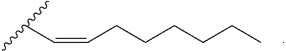 [SMILES: */C=C\CCCCCC]

In some embodiments, ionizable lipids of the present disclosure have a structure of Formula (S-I), Formula (S-Ia), or Formula (S-Ib), wherein R² is

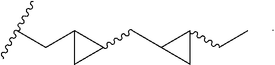 [SMILES: *CC1CC1CC1CC1CC]

### R³

In some embodiments, ionizable lipids of the present disclosure have a structure of Formula (S-I) or Formula (S-Ib), wherein R³ is hydrogen.

In some embodiments, ionizable lipids of the present disclosure have a structure of Formula (S-I), Formula (S-Ia), or Formula (S-Ib), wherein R³ is C₁₋₆alkylene. In some embodiments, ionizable lipids of the present disclosure have a structure of Formula (S-I), wherein each R³ is C₁alkyl, C₂alkyl, C₃alkyl, C₄alkyl, C₅alkyl, or C₆alkyl.

n

In some embodiments, ionizable lipids of the present disclosure have a structure of Formula (S-I), Formula (S-Ia), or Formula (S-Ib), wherein n is 3. In some embodiments, ionizable lipids of the present disclosure have a structure of Formula (S-I), Formula (S-Ia), or Formula (S-Ib), wherein n is 4. In some embodiments, ionizable lipids of the present disclosure have a structure of Formula (S-I), Formula (S-Ia), or Formula (S-Ib), wherein n is 1, 2, 5, or 6.

m

In some embodiments, ionizable lipids of the present disclosure have a structure of Formula (S-Ia), or Formula (S-Ib), wherein m is selected from 5 to 8. In some embodiments, ionizable lipids of the present disclosure have a structure of Formula (S-Ia), or Formula (S-Ib), wherein m is 5. In some embodiments, ionizable lipids of the present disclosure have a structure of Formula (S-Ia) or Formula (S-Ib), wherein m is 6. In some embodiments, ionizable lipids of the present disclosure have a structure of Formula (S-Ia) or Formula (S-Ib), wherein m is 7. In some embodiments, ionizable lipids of the present disclosure have a structure of Formula (S-Ia) or Formula (S-Ib), wherein m is 8. In some embodiments, ionizable lipids of the present disclosure have a structure of Formula (S-Ia), or Formula (S-Ib), wherein m is 2, 3, 4, 9, or 10.

p

In some embodiments, ionizable lipids of the present disclosure have a structure of Formula (S-I), Formula (S-Ia), or Formula (S-Ib), wherein p is independently selected from 2 to 4. In some embodiments, ionizable lipids of the present disclosure have a structure of Formula (S-I), Formula (S-Ia), or Formula (S-Ib), wherein p is 2. In some embodiments, ionizable lipids of the present disclosure have a structure of Formula (S-I), Formula (S-Ia), or Formula (S-Ib), wherein p is 3. In some embodiments, ionizable lipids of the present disclosure have a structure of Formula (S-I), Formula (S-Ia), or Formula (S-Ib), wherein p is 4. In some embodiments, ionizable lipids of the present disclosure have a structure of Formula (S-I), Formula (S-Ia), or Formula (S-Ib), wherein p is 5 or 6. In some embodiments, ionizable lipids of the present disclosure have a structure of Formula (S-I), wherein p is 1.

In some embodiments, ionizable lipids of the present disclosure have a structure of Formula (S-M):

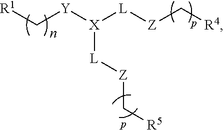

or a pharmaceutically acceptable salt thereof, wherein:

X is N or CH;
Y is a bond,

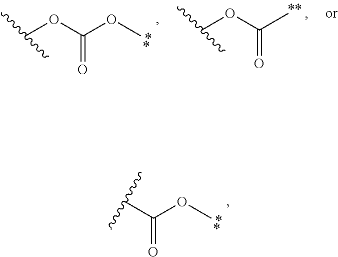

wherein bond marked with an “**” is attached to X;
each Z is independently selected from the group consisting of:

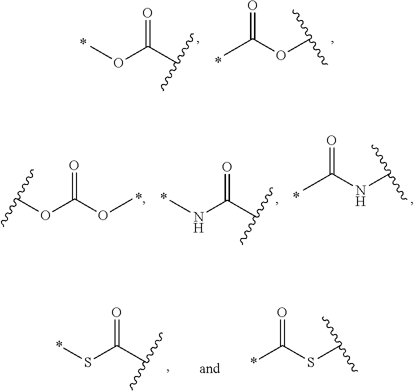

wherein the bond marked with an “*” is attached to L;
each L is independently C₂-C₁₀ alkylenyl;
R¹ is OH, N(R³)₂,

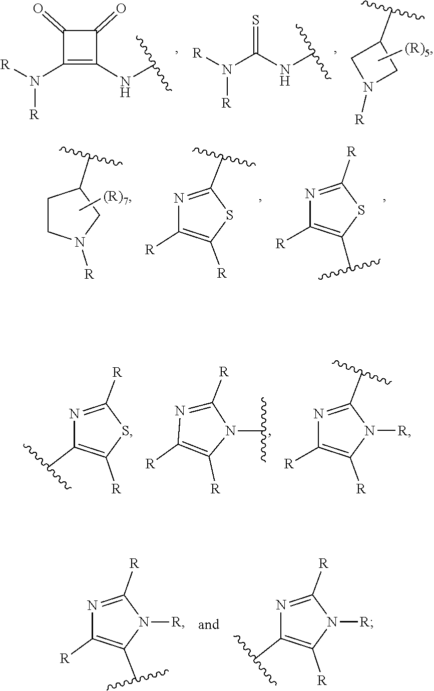

each R is independently —H or C₁-C₆ aliphatic;
each R³ independently selected from is H and C₁₋₆alkyl;
R⁴ is —CH(SR⁶)(SR⁷);
R⁵ is —CH(OR⁸)(OR⁹); —CH(SR⁸)(SR⁹); —CH(R⁸)(R⁹) or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—; and
R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
n is selected from 1 to 6; and
each p is independently selected from 1 to 6.

In some embodiments, ionizable lipids of the present disclosure have a structure of Formula (S-Ma)

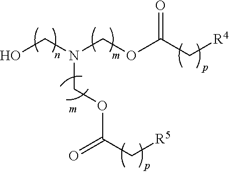

or a pharmaceutically acceptable salt thereof, wherein:
n is selected from 1 to 4;
each m is independently selected from 2 to 10; and
each p is independently selected from 2 to 6.

In some embodiments, ionizable lipids of the present disclosure have a structure of Formula (S-Mb)

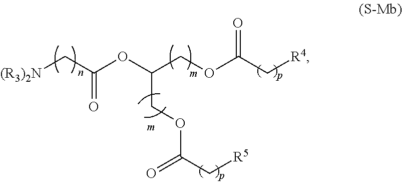

or a pharmaceutically acceptable salt thereof, wherein:
each R³ independently selected from is H and C₁₋₆ alkyl;
n is selected from 1 to 4;
each m is independently selected from 2 to 10; and
each p is independently selected from 2 to 6.

### R¹

In some embodiments, ionizable lipids of the present disclosure have a structure of Formula (S-M), wherein R¹ is OH. In some embodiments, ionizable lipids of the present disclosure have a structure of Formula (S-I), wherein R¹ is N(R³)₂. In some embodiments, ionizable lipids of the present disclosure have a structure of Formula (S-M), wherein R¹ is

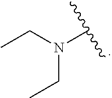 [SMILES: *N(CC)CC]

In some embodiments, ionizable lipids of the present disclosure have a structure of Formula (S-M), wherein R¹ is

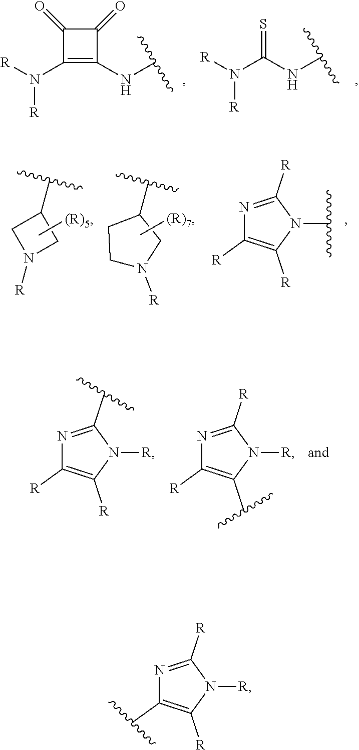

wherein each R is independently —H or C₁-C₆ aliphatic. In certain embodiments, R¹ is

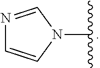 [SMILES: *n1ccnc1]

n

In some embodiments, ionizable lipids of the present disclosure have a structure of Formula (S-M), Formula (S-Ma), or Formula (S-Mb), wherein n is 3. In some embodiments, ionizable lipids of the present disclosure have a structure of Formula (S-M), Formula (S-Ma), or Formula (S-Mb), wherein n is 4. In some embodiments, ionizable lipids of the present disclosure have a structure of Formula (S-M), Formula (S-Ma), or Formula (S-Mb), wherein n is 1, 2, 5, or 6.

p

In some embodiments, ionizable lipids of the present disclosure have a structure of Formula (S-M), Formula (S-Ma), or Formula (S-Mb), wherein p is independently selected from 2 to 4. In some embodiments, ionizable lipids of the present disclosure have a structure of Formula (S-M), Formula (S-Ma), or Formula (S-Mb), wherein p is 2. In some embodiments, ionizable lipids of the present disclosure have a structure of Formula (S-M), Formula (S-Ma), or Formula (S-Mb), wherein p is 3. In some embodiments, ionizable lipids of the present disclosure have a structure of Formula (S-M), Formula (S-Ma), or Formula (S-Mb), wherein p is 4. In some embodiments, ionizable lipids of the present disclosure have a structure of Formula (S-M), Formula (S-Ma), or Formula (S-Mb), wherein p is 5 or 6. In some embodiments, ionizable lipids of the present disclosure have a structure of Formula (S-M), wherein p is 1.

### R⁵

As disclosed in Formula (S-M), in certain embodiments, R⁵ is —CH(OR⁸)(OR⁹); —CH(SR⁸)(SR⁹); —CH(R⁸)(R⁹) or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SR⁸)(SR⁹). In certain embodiments, R⁴ and R⁵ are the same. In certain embodiments, R⁴ and R⁵ are different.

In certain embodiments, R⁵ is selected from

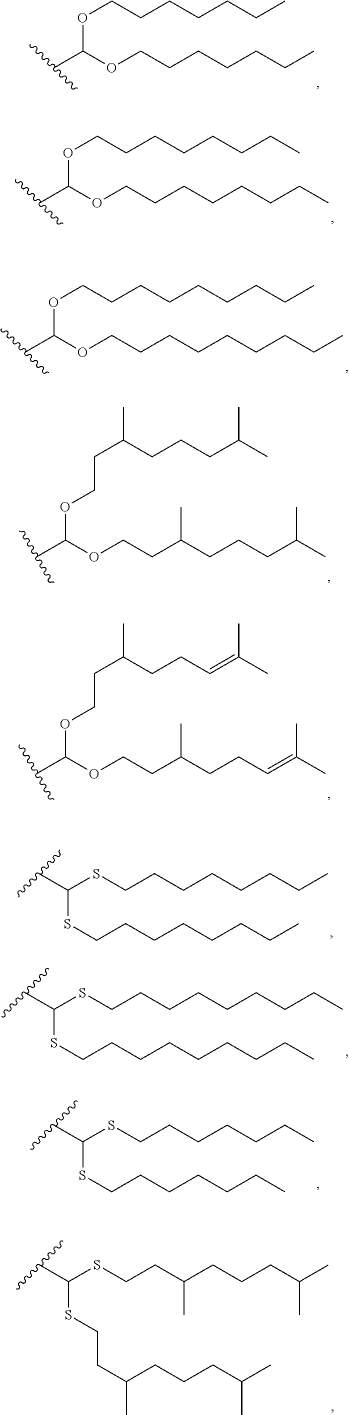

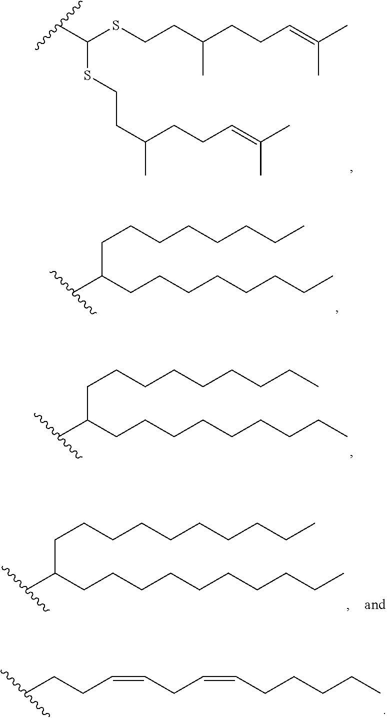 [SMILES: *C(SCCC(C)CCC=C(C)C)SCCC(C)CCC=C(C)C ; *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

R⁶ and R⁷

As disclosed in Formula (S-M), in certain embodiments, R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkylene. In certain embodiments, R⁶ is optionally substituted C₁-C₁₄ branched alkylene. In certain embodiments, R⁶ is optionally substituted C₁-C₁₄ straight chain alkylene. In certain embodiments, R⁶ is optionally substituted C₁-C₁₄ alkenylene. In certain embodiments, R⁶ is optionally substituted C₁-C₁₄ branched alkenylene. In certain embodiments, R⁶ is optionally substituted C₁-C₁₄ straight chain alkenylene. In certain embodiments, R⁶ is optionally substituted C₆-C₁₀ alkylene. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)₇CH₃. In certain embodiments, R⁶ is optionally substituted —(CH₂)₈CH₃. In certain embodiments, R⁶ is optionally substituted —(CH₂)₉CH₃.

In certain embodiments, one of the methylene linkages of R⁶ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

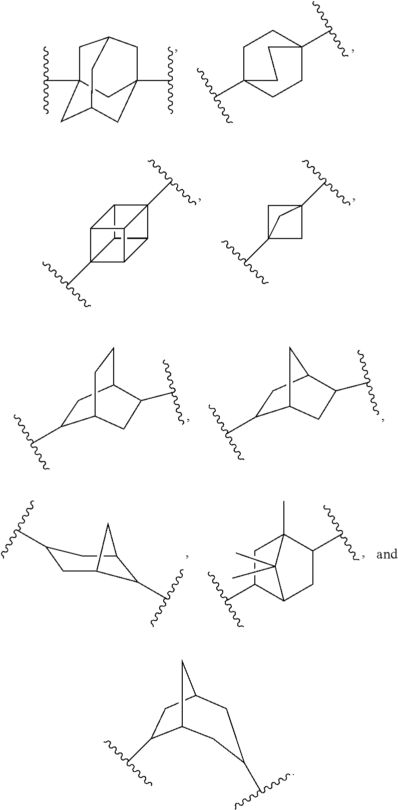

In certain embodiments, R⁷ is optionally substituted C₁-C₁₄ aliphatic. In certain embodiments, R⁷ is optionally substituted C₁-C₁₄ alkylene. In certain embodiments, R⁷ is optionally substituted C₁-C₁₄ branched alkylene. In certain embodiments, R⁷ is optionally substituted C₁-C₁₄ straight chain alkylene. In certain embodiments, R⁷ is optionally substituted C₁-C₁₄ alkenylene. In certain embodiments, R⁷ is optionally substituted C₁-C₁₄ branched alkenylene. In certain embodiments, R⁷ is optionally substituted C₁-C₁₄ straight chain alkenylene. In certain embodiments, R⁷ is optionally substituted C₆-C₁₀ alkylene. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁶ is optionally substituted —(CH₂)₉CH₃.

In certain embodiments, one of the methylene linkages of R⁷ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

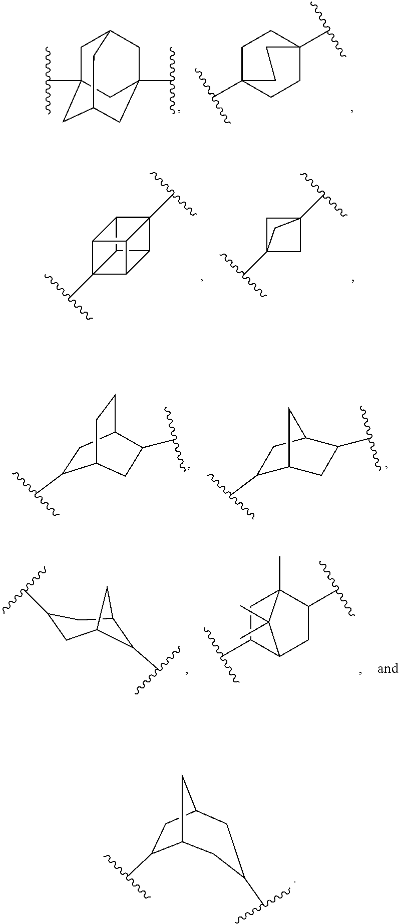

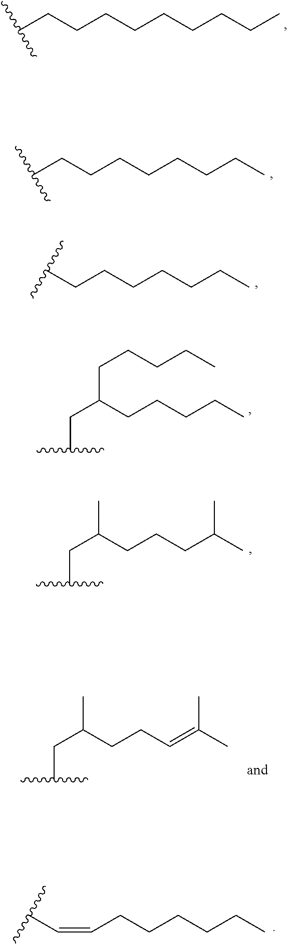

In certain embodiments, each R⁶ and R⁷ are each independently selected from an optionally substituted bridged bicyclic C₅-C₁₂ cycloalkylenyl. In certain embodiments, R⁶ is an optionally substituted bridged multicyclic C₅-C₁₂ cycloalkylenyl. In certain embodiments, R⁷ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

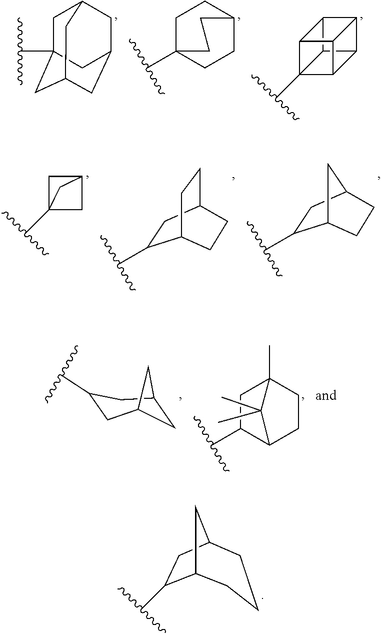

In certain embodiments, the substituted bridged bicyclic or multicyclic C₅-C₁₂ cycloalkylenyl is a structure selected from

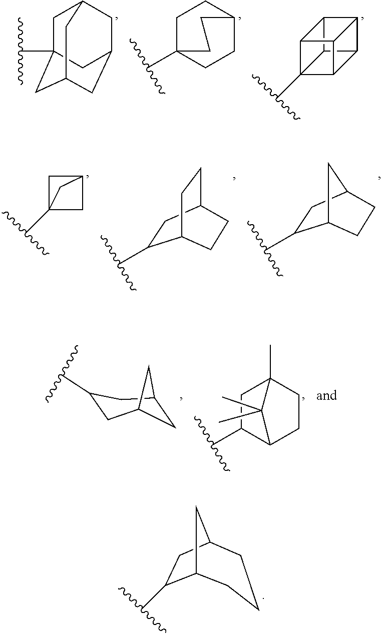

wherein one or more C—H bonds are substituted.

In certain embodiments, R⁶ and R⁷ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

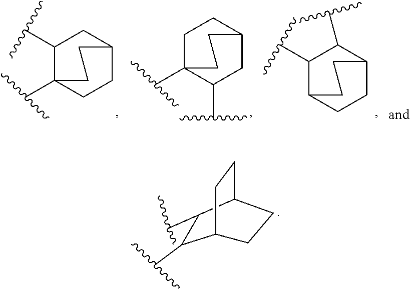

R⁸ and R⁹

As disclosed in Formula (S-M), in certain embodiments, R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is optionally substituted C₁-C₁₄ aliphatic. In certain embodiments, R⁸ is optionally substituted C₁-C₁₄ alkylene. In certain embodiments, R⁸ is optionally substituted C₁-C₁₄ branched alkylene. In certain embodiments, R⁸ is optionally substituted C₁-C₁₄ straight chain alkylene. In certain embodiments, R⁸ is optionally substituted C₁-C₁₄ alkenylene. In certain embodiments, R⁸ is optionally substituted C₁-C₁₄ branched alkenylene. In certain embodiments, R⁸ is optionally substituted C₁-C₁₄ straight chain alkenylene. In certain embodiments, R⁸ is optionally substituted C₆-C₁₀ alkylene. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, one of the methylene linkages of R⁸ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

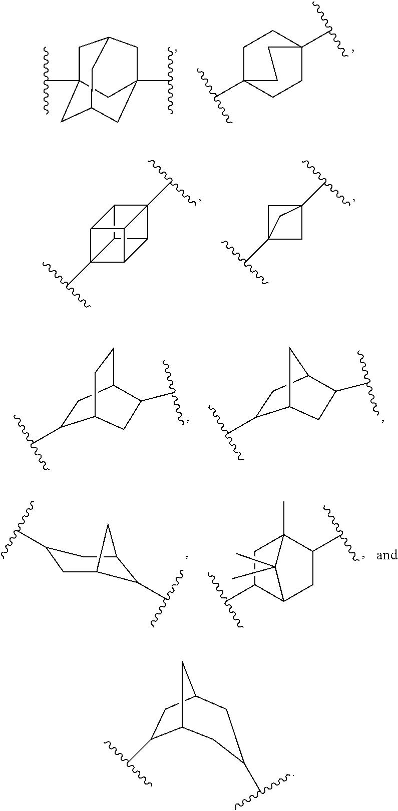

In certain embodiments, R⁹ is optionally substituted C₁-C₁₄ aliphatic. In certain embodiments, R⁹ is optionally substituted C₁-C₁₄ alkylene. In certain embodiments, R⁹ is optionally substituted C₁-C₁₄ branched alkylene. In certain embodiments, R⁹ is optionally substituted C₁-C₁₄ straight chain alkylene. In certain embodiments, R⁹ is optionally substituted C₁-C₁₄ alkenylene. In certain embodiments, R⁹ is optionally substituted C₁-C₁₄ branched alkenylene. In certain embodiments, R⁹ is optionally substituted C₁-C₁₄ straight chain alkenylene. In certain embodiments, R⁹ is optionally substituted C₆-C₁₀ alkylene. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, one of the methylene linkages of R⁹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

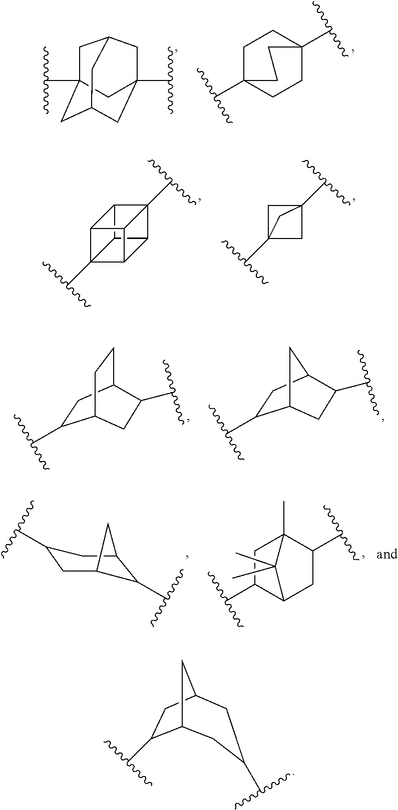

In certain embodiments, R⁸ and R⁹ are selected from

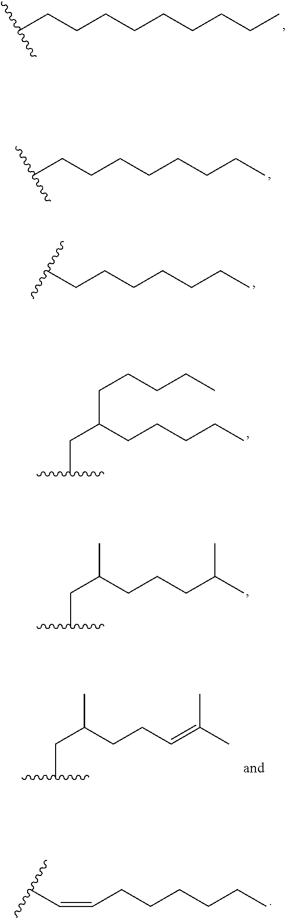

In some embodiments, R⁸ and R⁹ taken together form an optionally substituted bridged bicyclic or multicyclic C₄-C₁₄ cycloalkyl or optionally substituted bridged bicyclic or multicyclic 4-14 membered heterocyclyl.

In certain embodiments, each R⁸ and R⁹ are each independently selected from an optionally substituted bridged bicyclic C₅-C₁₂ cycloalkylenyl. In certain embodiments, R⁸ is an optionally substituted bridged multicyclic C₅-C₁₂ cycloalkylenyl. In certain embodiments, R⁹ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

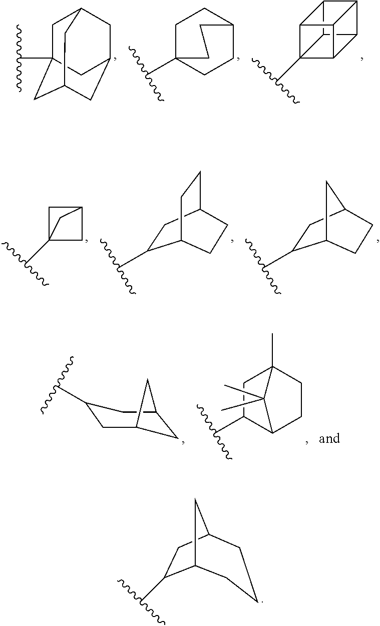

In certain embodiments, the substituted bridged bicyclic or multicyclic C₅-C₁₂ cycloalkylenyl is a structure selected from

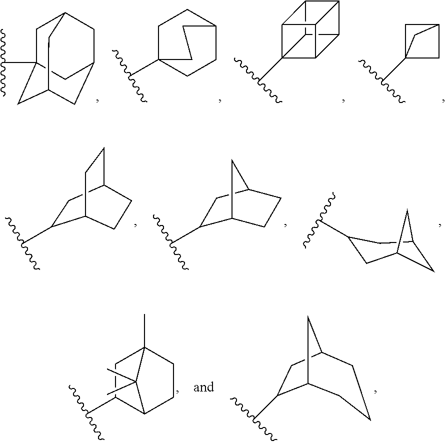

wherein one or more C—H bonds are substituted.

In certain embodiments, R⁸ and R⁹ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

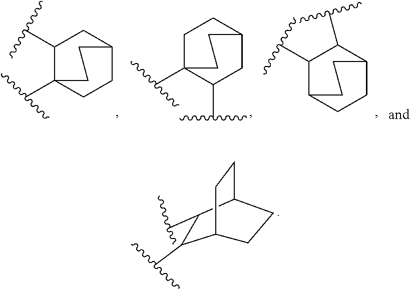

In some embodiments, ionizable lipids of the present disclosure comprise an acyclic core. In some embodiments, ionizable lipids of the present disclosure are selected from any lipid in Table (VI) below or a pharmaceutically acceptable salt thereof:

TABLE VI
Non-Limiting Examples of Ionizable Lipids

| # | Structure |
|---|---|
| S-1 | 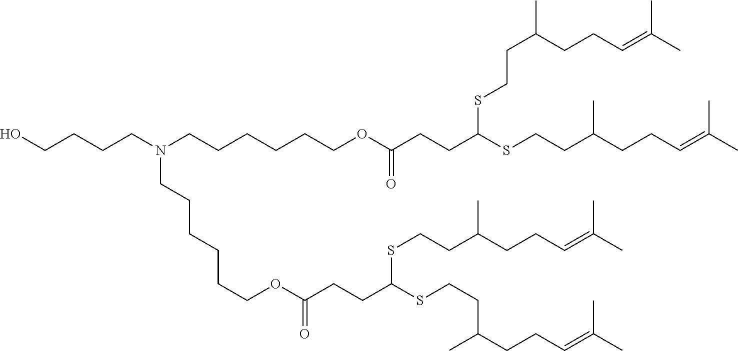 [SMILES: CC(C)=CCCC(C)CCSC(CCC(=O)OCCCCCCN(CCCCO)CCCCCCOC(=O)CCC(SCCC(C)CCC=C(C)C)SCCC(C)CCC=C(C)C)SCCC(C)CCC=C(C)C] |
| S-2 | 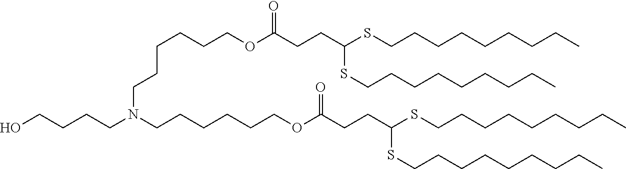 [SMILES: CCCCCCCCCSC(CCC(=O)OCCCCCCN(CCCCO)CCCCCCOC(=O)CCC(SCCCCCCCCC)SCCCCCCCCC)SCCCCCCCCC] |
| S-3 | 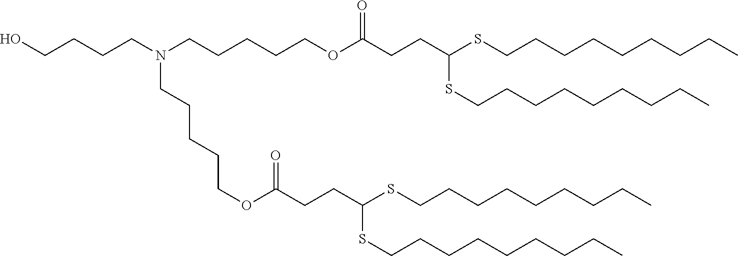 [SMILES: CCCCCCCCCSC(CCC(=O)OCCCCCN(CCCCO)CCCCCOC(=O)CCC(SCCCCCCCCC)SCCCCCCCCC)SCCCCCCCCC] |
| S-4 | 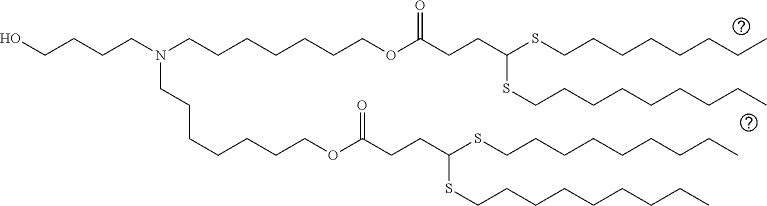 [SMILES: CCCCCCCCCSC(CCC(=O)OCCCCCCCN(CCCCO)CCCCCCCOC(=O)CCC(SCCCCCCCCC)SCCCCCCCCC)SCCCCCCCC] |
| S-5 | 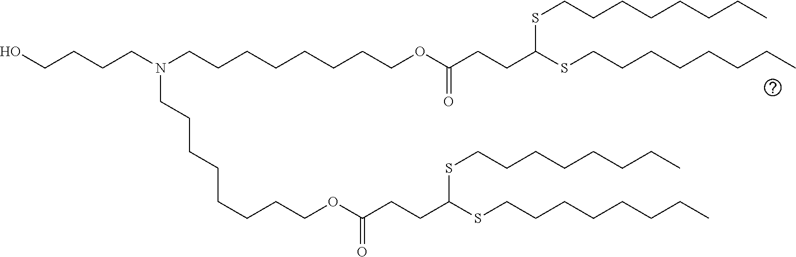 [SMILES: CCCCCCCCSC(CCC(=O)OCCCCCCCCN(CCCCO)CCCCCCCCOC(=O)CCC(SCCCCCCCC)SCCCCCCCC)SCCCCCCCC] |
| S-6 | 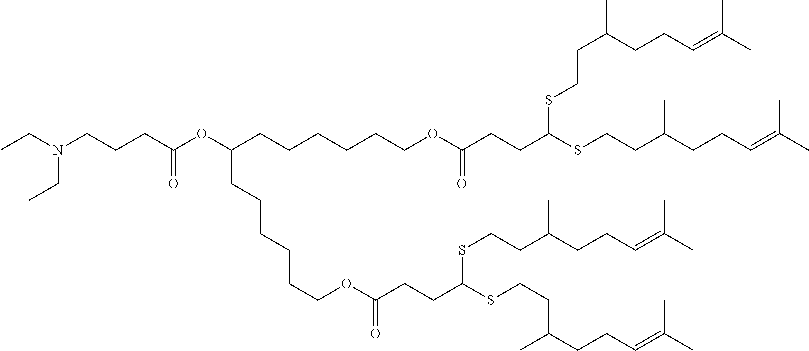 [SMILES: CCN(CC)CCCC(=O)OC(CCCCCCOC(=O)CCC(SCCC(C)CCC=C(C)C)SCCC(C)CCC=C(C)C)CCCCCCOC(=O)CCC(SCCC(C)CCC=C(C)C)SCCC(C)CCC=C(C)C] |
| S-7 | 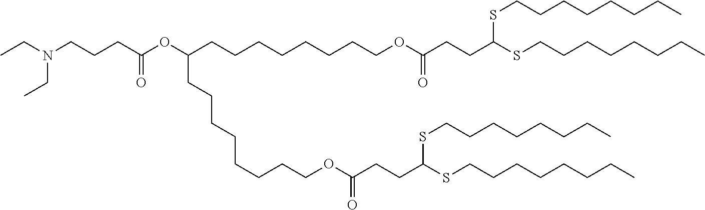 [SMILES: CCCCCCCCSC(CCC(=O)OCCCCCCCCC(CCCCCCCCOC(=O)CCC(SCCCCCCCC)SCCCCCCCC)OC(=O)CCCN(CC)CC)SCCCCCCCC] |
| S-8 | 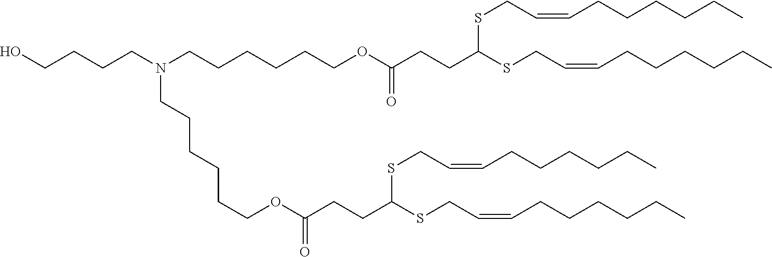 [SMILES: CCCCCC/C=C\CSC(CCC(=O)OCCCCCCN(CCCCO)CCCCCCOC(=O)CCC(SC/C=C\CCCCCC)SC/C=C\CCCCCC)SC/C=C\CCCCCC] |
| S-9 | 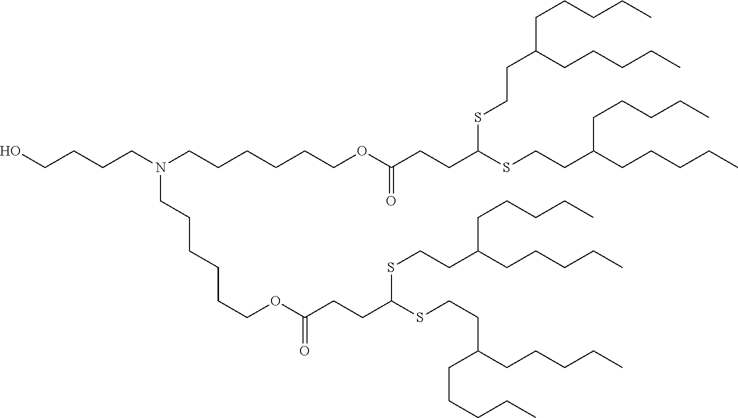 [SMILES: CCCCCC(CCCCC)CCSC(CCC(=O)OCCCCCCN(CCCCO)CCCCCCOC(=O)CCC(SCCC(CCCCC)CCCCC)SCCC(CCCCC)CCCCC)SCCC(CCCCC)CCCCC] |
| S-10 | 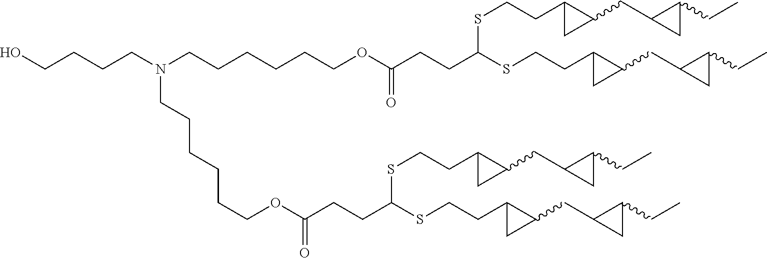 [SMILES: CCC1CC1CC1CC1CCSC(CCC(=O)OCCCCCCN(CCCCO)CCCCCCOC(=O)CCC(SCCC1CC1CC1CC1CC)SCCC1CC1CC1CC1CC)SCCC1CC1CC1CC1CC] |
| S-11 | 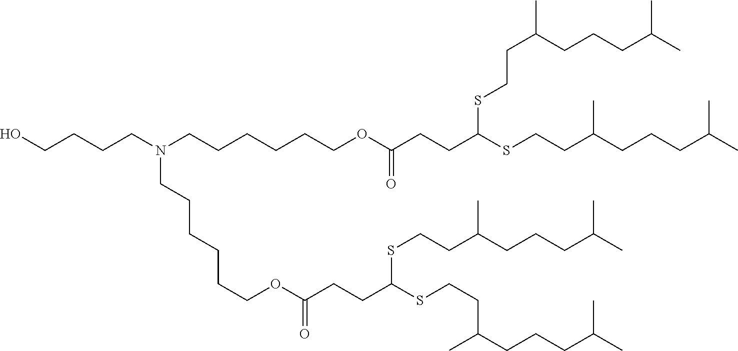 [SMILES: CC(C)CCCC(C)CCSC(CCC(=O)OCCCCCCN(CCCCO)CCCCCCOC(=O)CCC(SCCC(C)CCCC(C)C)SCCC(C)CCCC(C)C)SCCC(C)CCCC(C)C] |

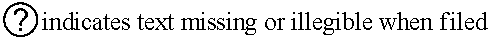

### Series “AT”

In some embodiments, an LNP of the present disclosure comprises an ionizable lipid disclosed in PCT Application PCT/US2024/019990, which is incorporated by reference herein, in its entirety.

In some embodiments, ionizable lipids of the present disclosure have a structure of Formula (AT)

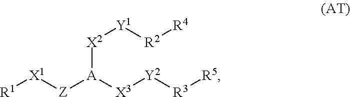

or a pharmaceutically acceptable salt thereof,
wherein:
i) A is N; Z is a bond; X¹ is optionally substituted C₁-C₆ aliphatic, wherein the optional substituent is not oxo when X¹ is C₁ aliphatic; and R¹ is selected from the group consisting of:

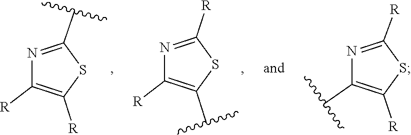

or
ii) A is CH;
Z is

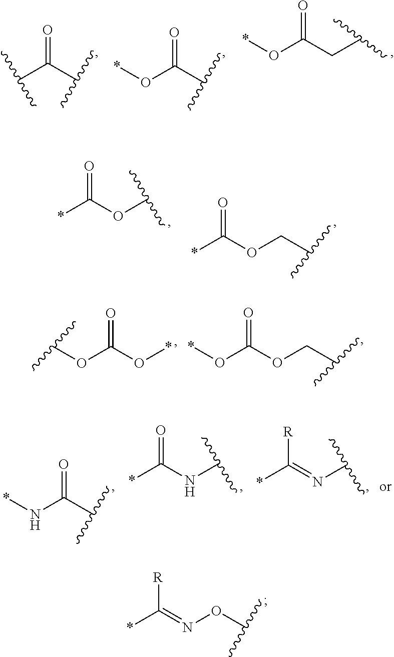

wherein the bond marked with an “*” is attached to X¹;
X¹ is a bond or optionally substituted C₁-C₆ aliphatic;
R¹ is selected from the group consisting of:

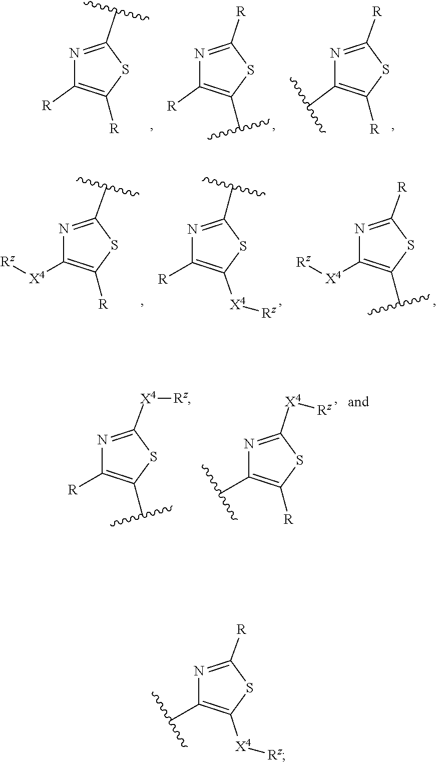

X⁴ is a bond or optionally substituted C₁-C₆ aliphatic;
R^{Z} is NR₂ or OH;
each R is independently —H or C₁-C₆ aliphatic;
X² and X³ are each independently optionally substituted C₁-C₁₂ aliphatic;
Y¹ and Y² are independently selected from the group consisting of

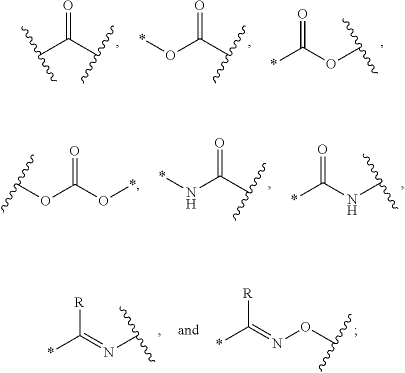

wherein the bond marked with an “*” is attached to X² for Y¹ or X³ for Y²;
R² is optionally substituted C₁-C₆ aliphatic;
R³ is optionally substituted C₁-C₆ aliphatic;
R⁴ is —CH(OR⁶)(OR⁷), —CH(SR⁶)(SR⁷), —CH(R⁶)(R⁷), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁵ is —CH(OR⁸)(OR⁹), —CH(SR⁸)(SR⁹), —CH(R⁸)(R⁹), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—; and
R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, ionizable lipids of the present disclosure have a structure of Formula (AT), wherein the ionizable lipids of the present disclosure have a structure of Formula (AT-E′):

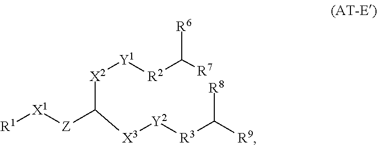

or a pharmaceutically acceptable salt thereof, wherein R¹, R, X¹, Z, X², X³, X⁴, R^{Z}, Y¹, Y², R², R³, R⁶, R⁷, R⁸, and R⁹ are as described in Formula (AT) or as otherwise described in any embodiments below.

In certain embodiments, ionizable lipids of the present disclosure have a structure of Formula (AT), wherein the ionizable lipids of the present disclosure have a structure of Formula (AT-F′″):

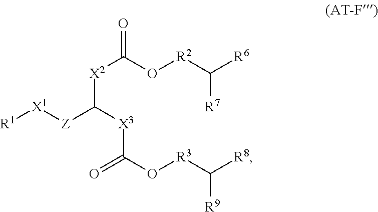

or a pharmaceutically acceptable salt thereof, wherein R¹, R, X¹, Z, X², X³, X⁴, R^{Z}, R², R³, R⁶, R⁷, R⁸, and R⁹ are as described in Formula (AT) or as otherwise described in any embodiments below.

In certain embodiments, ionizable lipids of the present disclosure have a structure of Formula (AT), wherein the ionizable lipids of the present disclosure have a structure of Formula (AT-M):

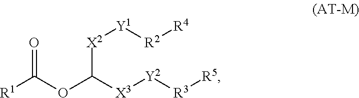

or a pharmaceutically acceptable salt thereof, wherein R¹, R, X², X³, X⁴, R^{Z}, Y¹, Y², R², R³, R⁴, R⁵, R⁶, R⁷, R⁸, and R⁹ are as described in Formula (AT) or as otherwise described in any embodiments below.

In certain embodiments, ionizable lipids of the present disclosure have a structure of Formula (AT), wherein the ionizable lipids of the present disclosure have a structure of Formula (AT-N′):

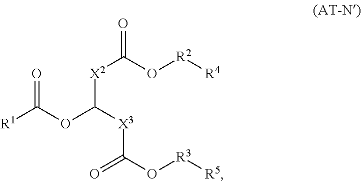

or a pharmaceutically acceptable salt thereof, wherein R¹, R, X¹, X², X³, X⁴, R^{Z}, R², R³, R⁴, R⁵, R⁶, R⁷, R⁸, and R⁹ are as described in Formula (AT) or as otherwise described in any embodiments below.

In certain embodiments, ionizable lipids of the present disclosure have a structure of Formula (AT), wherein the ionizable lipids of the present disclosure have a structure of Formula (AT-O′):

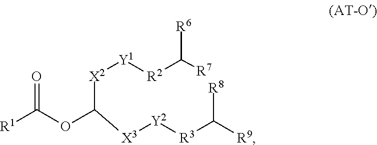

or a pharmaceutically acceptable salt thereof, wherein R¹, R, X¹, X², X³, Y¹, Y², X⁴, R^{Z}, R², R³, R⁶, R⁷, R⁸, and R⁹ are as described in Formula (AT) or as otherwise described in any embodiments below.

In certain embodiments, ionizable lipids of the present disclosure have a structure of Formula (AT), wherein the ionizable lipids of the present disclosure have a structure of Formula (AT-P′″):

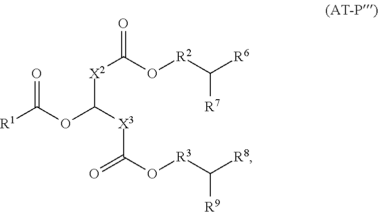

or a pharmaceutically acceptable salt thereof, wherein R¹, R, X¹, X², X³, R², R³, X⁴, R^{Z}, R⁶, R⁷, R⁸, and R⁹ are as described in Formula (AT) or as otherwise described in any embodiments below.

### A

As disclosed in Formula (AT), in certain embodiments, A is CH or N. In certain embodiments, A is CH. In certain embodiments, A is N.

### Z

As disclosed in Formula (AT), in certain embodiments wherein A is CH, Z is

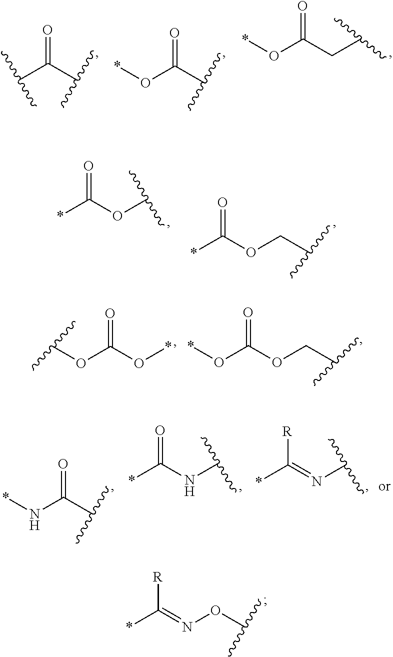

wherein the bond marked with an “*” is attached to X¹. In certain embodiments wherein A is CH, Z is

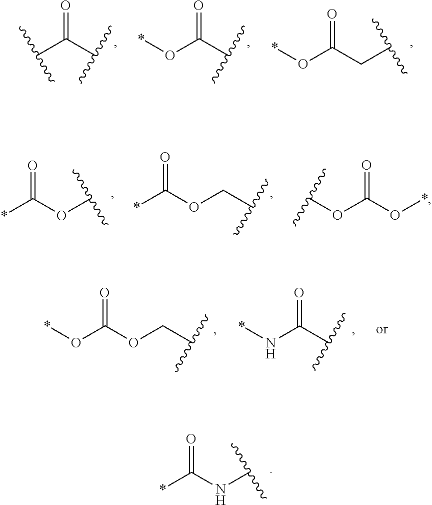

In certain embodiments wherein A is CH, Z is

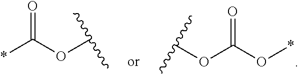 [SMILES: *OC(*)=O ; *OC(=O)O*]

In certain embodiments, Z is

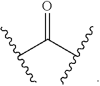 [SMILES: *C(*)=O]

In certain embodiments, Z is

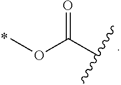 [SMILES: *C(=O)OC]

In certain embodiments, Z is

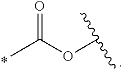 [SMILES: *OC(C)=O]

In certain embodiments, Z is

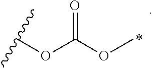

In certain embodiments, Z is

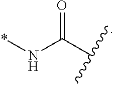 [SMILES: *NC(*)=O]

In certain embodiments, Z is

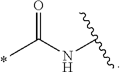

In certain embodiments, Z is

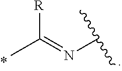

In certain embodiments, Z is

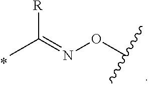

In certain embodiments, Z is

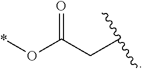 [SMILES: *CC(=O)O*]

In certain embodiments, Z is

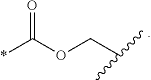 [SMILES: *COC(*)=O]

In certain embodiments, Z is

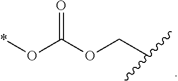 [SMILES: *COC(=O)O*]

As disclosed in Formula (AT), in certain embodiments wherein A is N, Z is a bond.

### X¹

As disclosed in Formula (AT), in certain embodiments wherein A is N, X¹ is optionally substituted C₁-C₆ aliphatic. In certain embodiments wherein A is N, X¹ is unsubstituted C₁-C₆ aliphatic. In certain embodiments, X¹ is optionally substituted C₁-C₆ alkylene. In certain embodiments, X¹ is unsubstituted C₁-C₆ alkylene. In certain embodiments, X¹ is unsubstituted C₂-C₆ alkylene. In certain embodiments, X¹ is optionally substituted methylene. In certain embodiments, R² is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)—. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

As disclosed in Formula (AT), in certain embodiments wherein A is CH, X¹ is a bond or optionally substituted C₁-C₆ aliphatic. In certain embodiments, X¹ is a bond. In certain embodiments, X¹ is optionally substituted C₁-C₆ alkylene. In certain embodiments, X¹ is unsubstituted C₁-C₆ alkylene. In certain embodiments, X¹ is unsubstituted C₂-C₆ alkylene. In certain embodiments, X¹ is optionally substituted methylene. In certain embodiments, R² is optionally substituted C₂ alkylene. In certain embodiments, X¹ is optionally substituted C₃ alkylene. In certain embodiments, X is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)—. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

### R¹

As disclosed in Formula (AT), in certain embodiments wherein A is N, R¹ is selected from the group consisting of

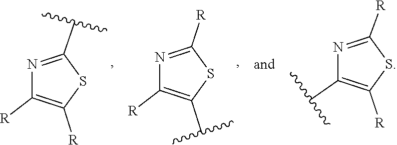

As disclosed in Formula (AT), in certain embodiments wherein A is CH, R¹ is selected from the group consisting of

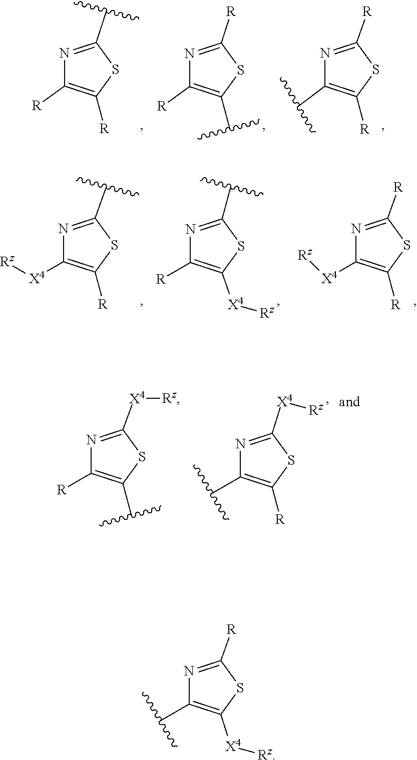

In certain embodiments, R¹ is

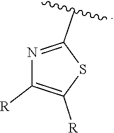

In certain embodiments, R¹ is

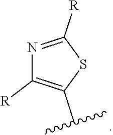

In certain embodiments, R¹ is

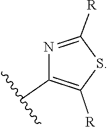

In certain embodiments, R¹ is

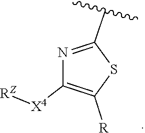

In certain embodiments, R¹ is

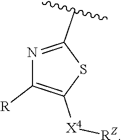

In certain embodiments, R¹ is

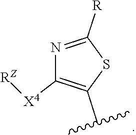

In certain embodiments, R¹ is

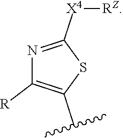

In certain embodiments, R¹ is

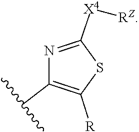

In certain embodiments, R¹ is

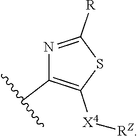

In certain embodiments, R¹ is

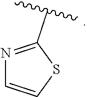 [SMILES: *c1nccs1]

In certain embodiments, R¹ is

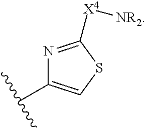

In certain embodiments, R¹ is

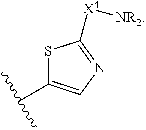

In certain embodiments, R¹ is

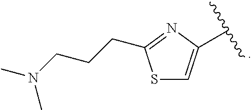 [SMILES: *c1csc(CCCN(C)C)n1]

X² and X³

As disclosed in Formula (AT), in certain embodiments, X² and X³ are each independently optionally substituted C₁-C₁₂ aliphatic. In certain embodiments, X² and X³ are the same. In certain embodiments, X² and X³ are different.

In certain embodiments, X² is an optionally substituted C₁-C₁₂ alkylene. In certain embodiments, X² is an optionally substituted C₁-C₁₂ alkenylene. In certain embodiments, X² is an optionally substituted C₁-C₁₀ aliphatic. In certain embodiments, X² is an optionally substituted C₁-C₁₀ alkylene. In certain embodiments, X² is an optionally substituted C₁-C₁₀ alkenylene. In certain embodiments, X² is an optionally substituted C₁-C₈ aliphatic. In certain embodiments, X² is an optionally substituted C₁-C₈ alkylene. In certain embodiments, X² is an optionally substituted C₁-C₈ alkenylene. In certain embodiments, X² is an optionally substituted C₁-C₆ aliphatic. In certain embodiments, X² is an optionally substituted C₁-C₆ alkylene. In certain embodiments, X² is an optionally substituted C₁-C₆ alkenylene. In certain embodiments, X² is an optionally substituted C₂-C₁₂ aliphatic. In certain embodiments, X² is an optionally substituted C₂-C₁₂ alkylene. In certain embodiments, X² is an optionally substituted C₂-C₁₂ alkenylene. In certain embodiments, X² is an optionally substituted C₄-C₁₂ aliphatic. In certain embodiments, X² is an optionally substituted C₄-C₁₂ alkylene. In certain embodiments, X² is an optionally substituted C₄-C₁₂ alkenylene. In certain embodiments, X² is an optionally substituted C₄-C₁₀ aliphatic. In certain embodiments, X² is an optionally substituted C₄-C₁₀ alkylene. In certain embodiments, X² is an optionally substituted C₄-C₁₀ alkenylene. In certain embodiments, X² is an optionally substituted C₆-C₈ aliphatic. In certain embodiments, X² is an optionally substituted C₆-C₈ alkylene. In certain embodiments, X² is an optionally substituted C₆-C₈ alkenylene. In certain embodiments, X² is —(CH₂)—. In certain embodiments, X² is —(CH₂)₂—. In certain embodiments, X² is —(CH₂)₃—. In certain embodiments, X² is —(CH₂)₄—. In certain embodiments, X² is —(CH₂)₅—. In certain embodiments, X² is —(CH₂)₆—. In certain embodiments, X² is —(CH₂)₇—. In certain embodiments, X² is —(CH₂)₈—. In certain embodiments, X² is —(CH₂)₉—. In certain embodiments, X² is —(CH₂)₁₀—.

In certain embodiments, X³ is an optionally substituted C₁-C₁₂ alkylene. In certain embodiments, X³ is an optionally substituted C₁-C₁₂ alkenylene. In certain embodiments, X³ is an optionally substituted C₁-C₁₀ aliphatic. In certain embodiments, X³ is an optionally substituted C₁-C₁₀ alkylene. In certain embodiments, X³ is an optionally substituted C₁-C₁₀ alkenylene. In certain embodiments, X³ is an optionally substituted C₁-C₈ aliphatic. In certain embodiments, X³ is an optionally substituted C₁-C₈ alkylene. In certain embodiments, X³ is an optionally substituted C₁-C₈ alkenylene. In certain embodiments, X³ is an optionally substituted C₁-C₆ aliphatic. In certain embodiments, X³ is an optionally substituted C₁-C₆ alkylene. In certain embodiments, X³ is an optionally substituted C₁-C₆ alkenylene. In certain embodiments, X³ is an optionally substituted C₂-C₁₂ aliphatic. In certain embodiments, X³ is an optionally substituted C₂-C₁₂ alkylene. In certain embodiments, X³ is an optionally substituted C₂-C₁₂ alkenylene. In certain embodiments, X³ is an optionally substituted C₄-C₁₂ aliphatic. In certain embodiments, X³ is an optionally substituted C₄-C₁₂ alkylene. In certain embodiments, X³ is an optionally substituted C₄-C₁₂ alkenylene. In certain embodiments, X³ is an optionally substituted C₄-C₁₀ aliphatic. In certain embodiments, X³ is an optionally substituted C₄-C₁₀ alkylene. In certain embodiments, X³ is an optionally substituted C₄-C₁₀ alkenylene. In certain embodiments, X³ is an optionally substituted C₆-C₈ aliphatic. In certain embodiments, X³ is an optionally substituted C₆-C₈ alkylene. In certain embodiments, X³ is an optionally substituted C₆-C₈ alkenylene. In certain embodiments, X³ is —(CH₂)—. In certain embodiments, X³ is —(CH₂)₂—. In certain embodiments, X³ is —(CH₂)₃—. In certain embodiments, X³ is —(CH₂)₄—. In certain embodiments, X³ is —(CH₂)₅—. In certain embodiments, X³ is —(CH₂)₆—. In certain embodiments, X³ is —(CH₂)₇—. In certain embodiments, X³ is —(CH₂)₈—. In certain embodiments, X³ is —(CH₂)₉—. In certain embodiments, X³ is —(CH₂)₁₀—.

In certain embodiments, X² and X³ are both —(CH₂)₈—. In certain embodiments, X² and X³ are both —(CH₂)₆—.

### X⁴

As disclosed in Formula (AT), in certain embodiments, X⁴ is a bond or C₂-C₆ aliphatic. In certain embodiments, X⁴ is a bond. In certain embodiments, X⁴ is C₂-C₆ aliphatic. In certain embodiments, X⁴ is C₂ aliphatic. In certain embodiments, X⁴ is C₃ aliphatic. In certain embodiments, X⁴ is C₄ aliphatic. In certain embodiments, X⁴ is C₅ aliphatic. In certain embodiments, X⁴ is C₆ aliphatic.

Y¹ and Y²

As disclosed in Formula (AT), in certain embodiments, Y¹ and Y² are each independently

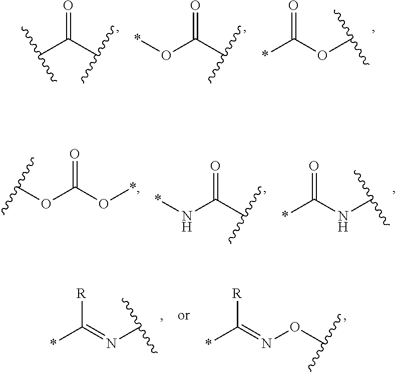

wherein the bond marked with an “*” is attached to X² for Y¹ or X³ for Y². In certain embodiments, Y¹ and Y² are the same. In certain embodiments, Y¹ and Y² are different.

In certain embodiments, Y¹ and Y² are each independently

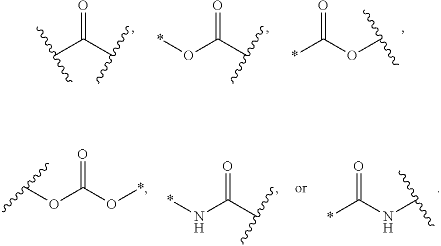

In certain embodiments, Y¹ and Y² are each independently

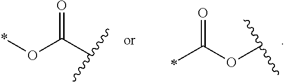

In certain embodiments, Y¹ is

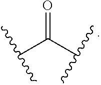 [SMILES: *C(*)=O]

In certain embodiments, Y¹ is

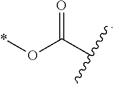 [SMILES: *OC(*)=O]

In certain embodiments, Y¹ is

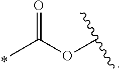

In certain embodiments, Y¹ is

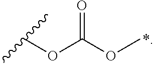

In certain embodiments, Y¹ is

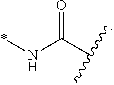 [SMILES: *NC(*)=O]

In certain embodiments, Y¹ is

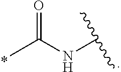

In certain embodiments, Y¹ is

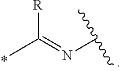

In certain embodiments, Y¹ is

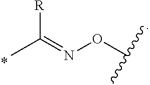

In certain embodiments, Y² is

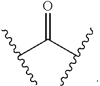 [SMILES: *C(*)=O]

In certain embodiments, Y² is

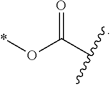 [SMILES: *OC(*)=O]

In certain embodiments, Y² is

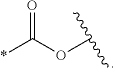 [SMILES: *OC(*)=O]

In certain embodiments, Y² is

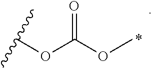

In certain embodiments, Y² is

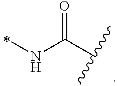

In certain embodiments, Y² is

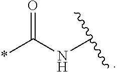 [SMILES: *NC(*)=O]

In certain embodiments, Y² is

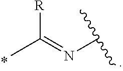

In certain embodiments, Y² is

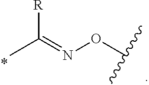

In certain embodiments, Y¹ and Y² are both

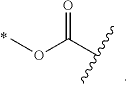 [SMILES: *C(=O)OC]

In certain embodiments, Y¹ and Y² are both

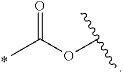 [SMILES: *OC(C)=O]

### R²

As disclosed in Formula (AT), in certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

As disclosed in Formula (AT), in certain embodiments, R³ is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different.

### R⁴

As disclosed in Formula (AT), in certain embodiments, R⁴ is —CH(OR⁶)(OR⁷), —CH(SR⁶)(SR⁷), —CH(R⁶)(R⁷), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic. In certain embodiments, R⁴ is —CH(OR⁶)(OR⁷). In certain embodiments, R⁴ is —CH(R⁶)(R⁷). In certain embodiments, R⁴ is —CH(SR⁶)(SR⁷).

In certain embodiments, one of the methylene linkages of R⁴ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

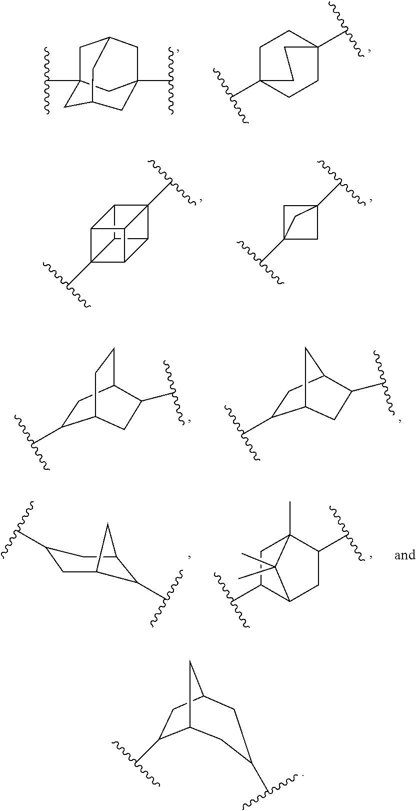

In certain embodiments, R⁴ is selected from is selected from

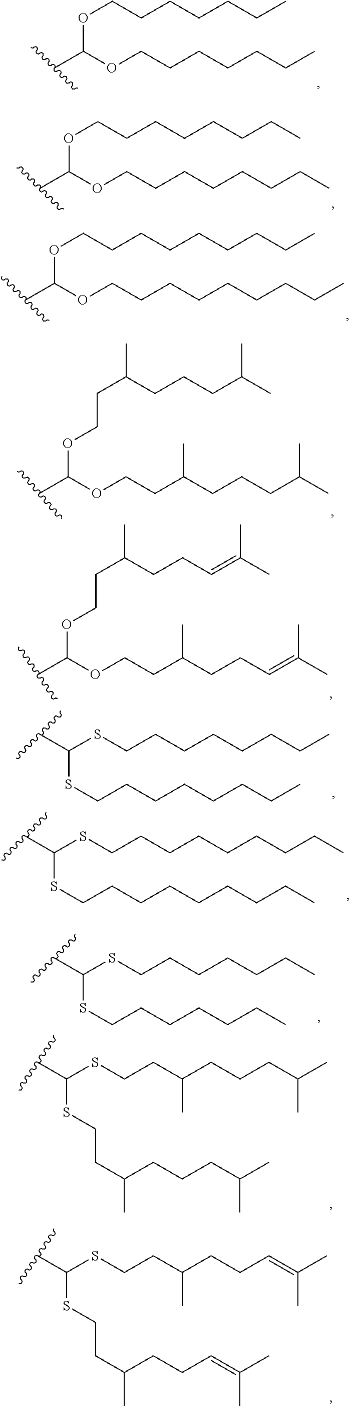

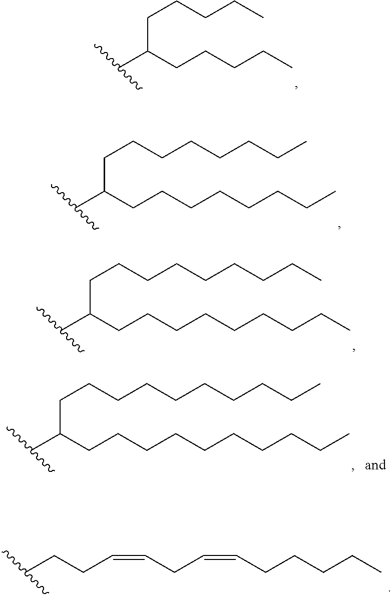 [SMILES: *C(CCCCC)CCCCC ; *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

In certain embodiments, R⁴ is selected from is selected from

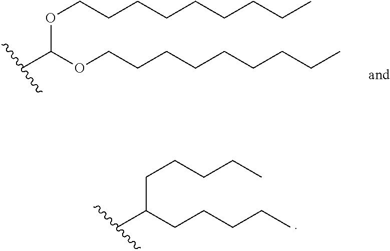 [SMILES: *C(OCCCCCCCCC)OCCCCCCCCC ; *C(CCCCC)CCCCC]

### R⁵

As disclosed in Formula (AT), in certain embodiments, R⁵ is —CH(OR⁸)(OR⁹), —CH(SR⁸)(SR⁹), —CH(R⁸)(R⁹), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SR⁸)(SR⁹).

In certain embodiments, one of the methylene linkages of R⁵ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

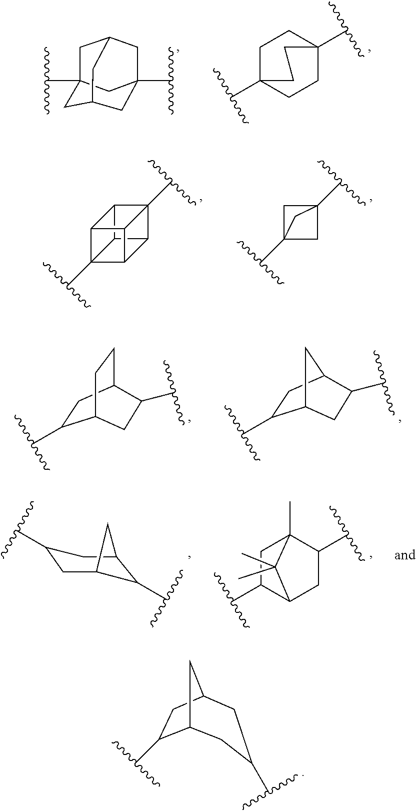

In certain embodiments, R⁴ and R⁵ are the same. In certain embodiments, R⁴ and R⁵ are different.

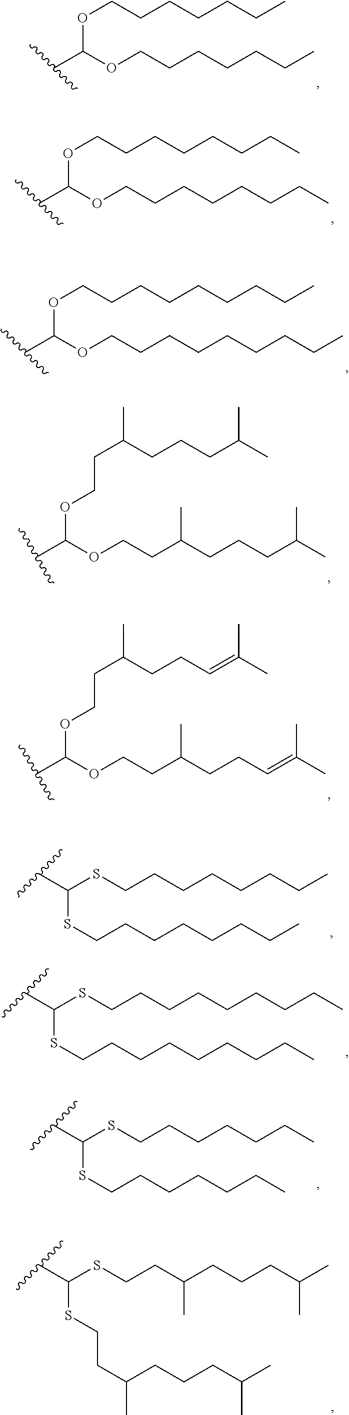

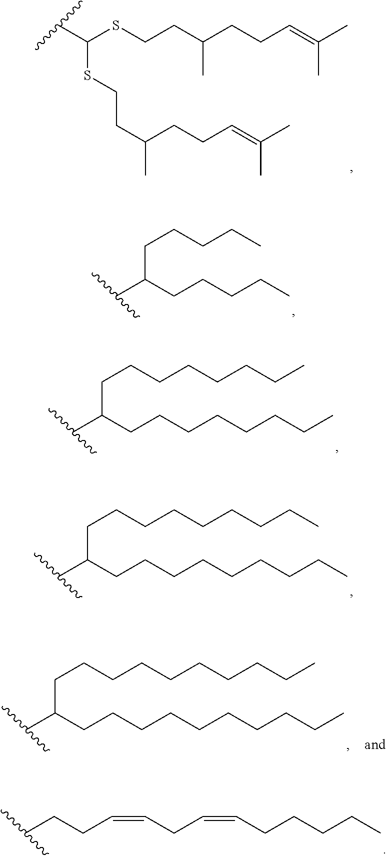 [SMILES: *C(SCCC(C)CCC=C(C)C)SCCC(C)CCC=C(C)C ; *C(CCCCC)CCCCC ; *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

In certain embodiments, R⁵ is selected from

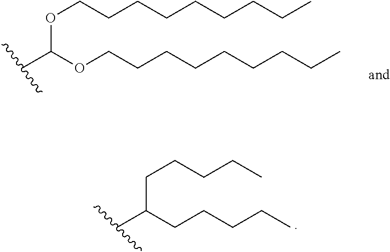 [SMILES: *C(OCCCCCCCCC)OCCCCCCCCC ; *C(CCCCC)CCCCC]

In certain embodiments, R⁵ is selected from is selected from R⁶ and R⁷

As disclosed in Formula (AT), in certain embodiments, R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkyl. In certain embodiments, R⁶ is optionally substituted C₁-C₁₄ branched alkyl. In certain embodiments, R⁶ is optionally substituted C₁-C₁₄ straight chain alkyl. In certain embodiments, R⁶ is optionally substituted C₁-C₁₄ alkenylene. In certain embodiments, R⁶ is optionally substituted C₁-C₁₄ branched alkenyl. In certain embodiments, R⁶ is optionally substituted C₁-C₁₄ straight chain alkenyl. In certain embodiments, R⁶ is optionally substituted C₆-C₁₀ alkyl. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)₇CH₃. In certain embodiments, R⁶ is optionally substituted —(CH₂)₈CH₃. In certain embodiments, R⁶ is optionally substituted —(CH₂)₉CH₃.

In certain embodiments, one of the methylene linkages of R⁶ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

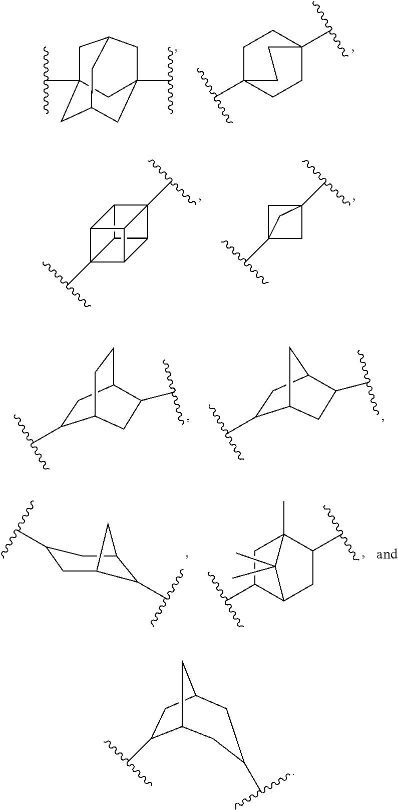

In certain embodiments, R⁷ is optionally substituted C₁-C₁₄ aliphatic. In certain embodiments, R⁷ is optionally substituted C₁-C₁₄ alkyl. In certain embodiments, R⁷ is optionally substituted C₁-C₁₄ branched alkyl. In certain embodiments, R⁷ is optionally substituted C₁-C₁₄ straight chain alkyl. In certain embodiments, R⁷ is optionally substituted C₁-C₁₄ alkenylene. In certain embodiments, R⁷ is optionally substituted C₁-C₁₄ branched alkenyl. In certain embodiments, R⁷ is optionally substituted C₁-C₁₄ straight chain alkenyl. In certain embodiments, R⁷ is optionally substituted C₆-C₁₀ alkyl. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁶ is optionally substituted —(CH₂)₉CH₃.

In certain embodiments, one of the methylene linkages of R⁷ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

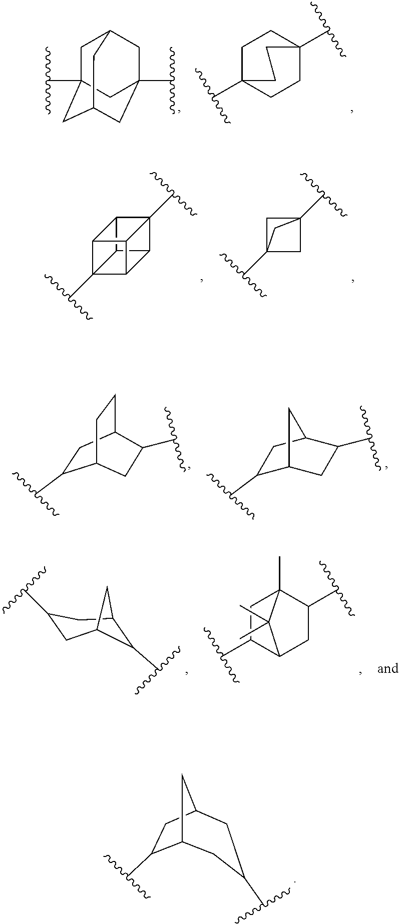

In certain embodiments, each R⁶ and R⁷ are selected from

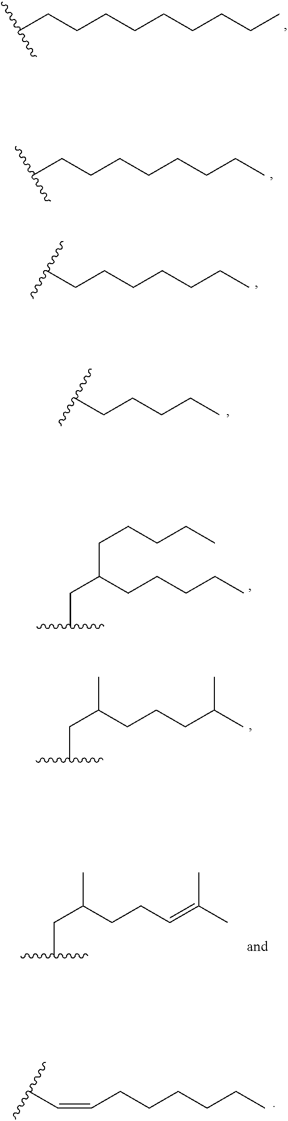

In certain embodiments, each R⁶ and R⁷ are each independently selected from an optionally substituted bridged bicyclic C₅-C₁₂ cycloalkylenyl. In certain embodiments, R⁶ is an optionally substituted bridged multicyclic C₅-C₁₂ cycloalkylenyl. In certain embodiments, R⁷ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

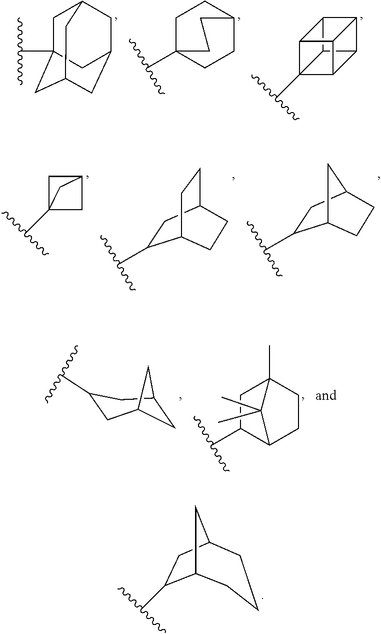

In certain embodiments, the substituted bridged bicyclic or multicyclic C₅-C₁₂ cycloalkylenyl is a structure selected from

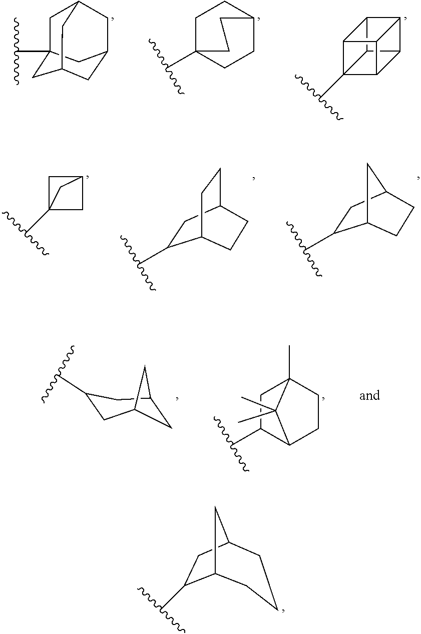

wherein one or more C—H bonds are substituted.

In certain embodiments, R⁶ and R⁷ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

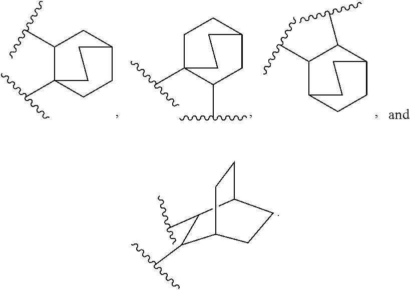

R⁸ and R⁹

As disclosed in Formula (AT), in certain embodiments, R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is optionally substituted C₁-C₁₄ aliphatic. In certain embodiments, R⁸ is optionally substituted C₁-C₁₄ alkyl. In certain embodiments, R⁸ is optionally substituted C₁-C₁₄ branched alkyl. In certain embodiments, R⁸ is optionally substituted C₁-C₁₄ straight chain alkyl. In certain embodiments, R⁸ is optionally substituted C₁-C₁₄ alkenyl. In certain embodiments, R⁸ is optionally substituted C₁-C₁₄ branched alkenyl. In certain embodiments, R⁸ is optionally substituted C₁-C₁₄ straight chain alkenyl. In certain embodiments, R⁸ is optionally substituted C₆-C₁₀ alkyl. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, one of the methylene linkages of R⁸ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

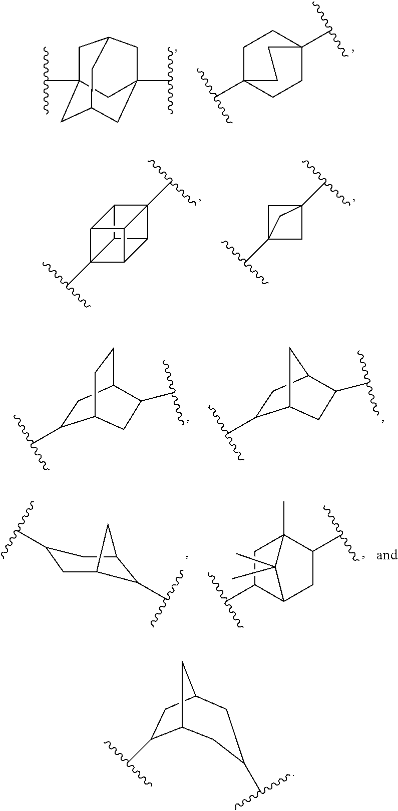

In certain embodiments, R⁹ is optionally substituted C₁-C₁₄ aliphatic. In certain embodiments, R⁹ is optionally substituted C₁-C₁₄ alkyl. In certain embodiments, R⁹ is optionally substituted C₁-C₁₄ branched alkyl. In certain embodiments, R⁹ is optionally substituted C₁-C₁₄ straight chain alkyl. In certain embodiments, R⁹ is optionally substituted C₁-C₁₄ alkenyl. In certain embodiments, R⁹ is optionally substituted C₁-C₁₄ branched alkenyl. In certain embodiments, R⁹ is optionally substituted C₁-C₁₄ straight chain alkenyl. In certain embodiments, R⁹ is optionally substituted C₆-C₁₀ alkyl. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, one of the methylene linkages of R⁹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

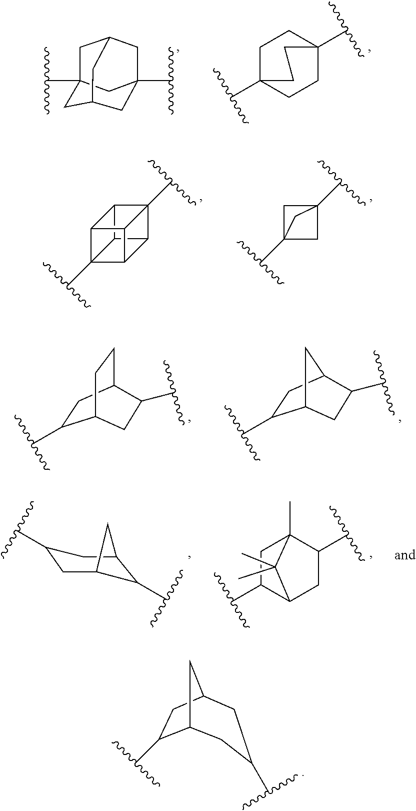

In certain embodiments, each R⁸ and R⁹ are selected from

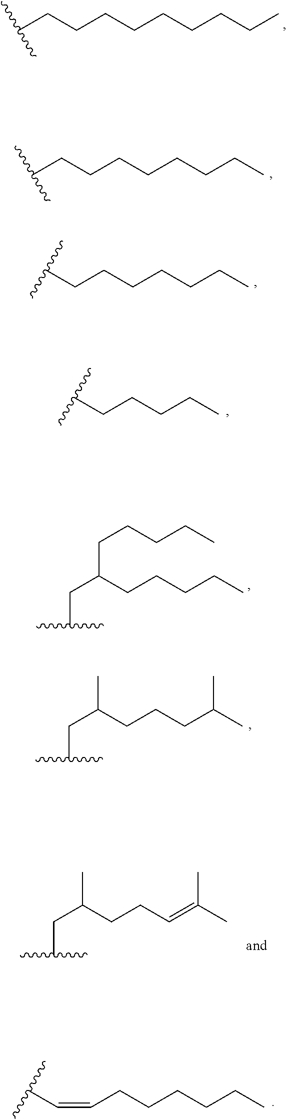

In some embodiments, R⁸ and R⁹ taken together form an optionally substituted bridged bicyclic or multicyclic C₄-C₁₄ cycloalkyl or optionally substituted bridged bicyclic or multicyclic 4-14 membered heterocyclyl.

In certain embodiments, each R⁸ and R⁹ are each independently selected from an optionally substituted bridged bicyclic C₅-C₁₂ cycloalkylenyl. In certain embodiments, R⁸ is an optionally substituted bridged multicyclic C₅-C₁₂ cycloalkylenyl. In certain embodiments, R⁹ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

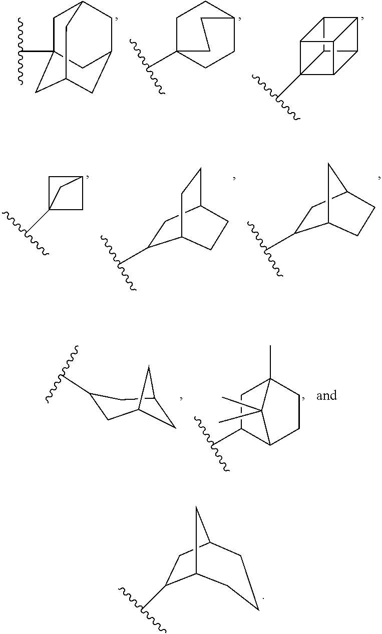

In certain embodiments, the substituted bridged bicyclic or multicyclic C₅-C₁₂ cycloalkylenyl is a structure selected from

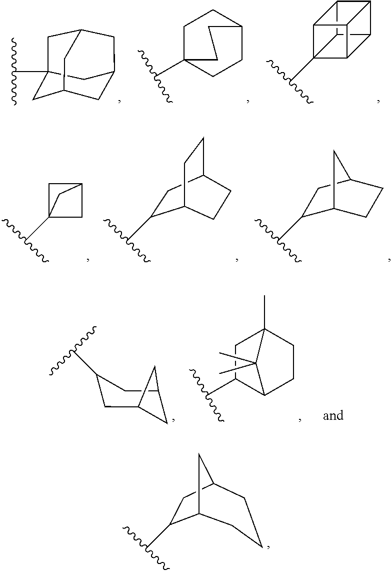

wherein one or more C—H bonds are substituted.

In certain embodiments, R⁸ and R⁹ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

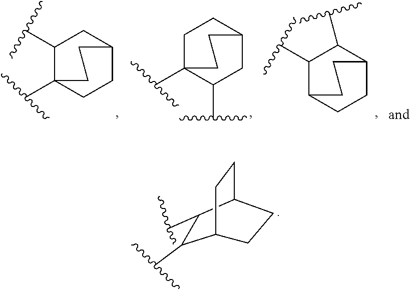

In some embodiments, Lipids of the Present Disclosure are selected from any lipid in Table (VII) below or a pharmaceutically acceptable salt thereof:

TABLE (VII)
Non-Limiting Examples of Ionizable Lipids of the Present Disclosure

| Cmpd | Structure |
|---|---|
| AT-1 | 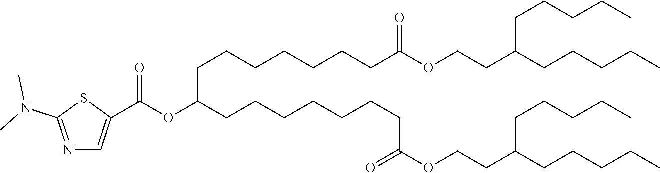 [SMILES: CCCCCC(CCCCC)CCOC(=O)CCCCCCCC(CCCCCCCC(=O)OCCC(CCCCC)CCCCC)OC(=O)c1cnc(N(C)C)s1] |
| AT-2 | 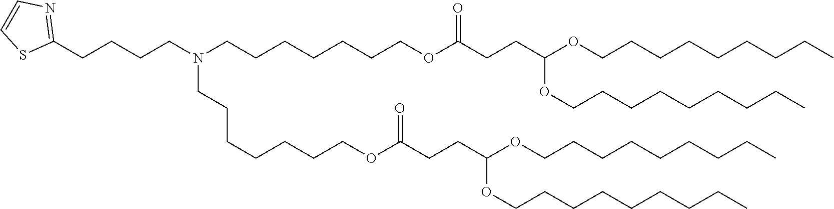 [SMILES: CCCCCCCCCOC(CCC(=O)OCCCCCCCN(CCCCCCCOC(=O)CCC(OCCCCCCCCC)OCCCCCCCCC)CCCCc1nccs1)OCCCCCCCCC] |
| AT-3 | 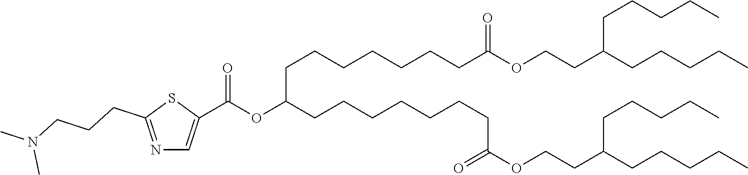 [SMILES: CCCCCC(CCCCC)CCOC(=O)CCCCCCCC(CCCCCCCC(=O)OCCC(CCCCC)CCCCC)OC(=O)c1cnc(CCCN(C)C)s1] |
| AT-4 | 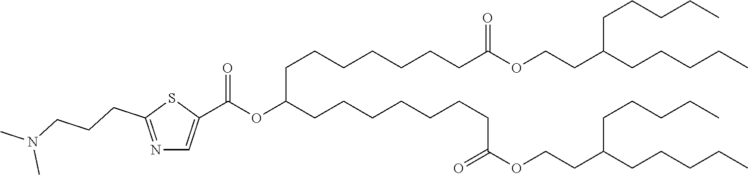 [SMILES: CCCCCC(CCCCC)CCOC(=O)CCCCCCCC(CCCCCCCC(=O)OCCC(CCCCC)CCCCC)OC(=O)c1cnc(CCCN(C)C)s1] |
| AT-5 | 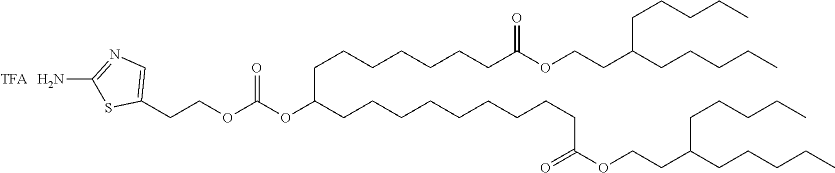 [SMILES: CCCCCC(CCCCC)CCOC(=O)CCCCCCCCCC(CCCCCCCC(=O)OCCC(CCCCC)CCCCC)OC(=O)OCCc1cnc(N)s1 ; "TFA"] |
| AT-6 | 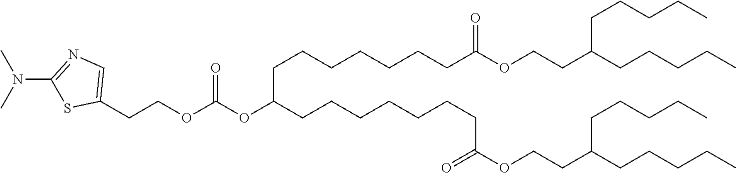 [SMILES: CCCCCC(CCCCC)CCOC(=O)CCCCCCCC(CCCCCCCC(=O)OCCC(CCCCC)CCCCC)OC(=O)OCCc1cnc(N(C)C)s1] |
| AT-7 | 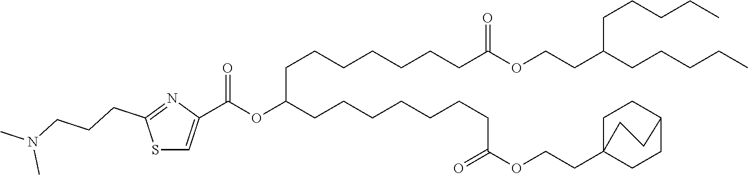 [SMILES: CCCCCC(CCCCC)CCOC(=O)CCCCCCCC(CCCCCCCC(=O)OCCC12CCC(CC1)CC2)OC(=O)c1csc(CCCN(C)C)n1] |
| AT-8 | 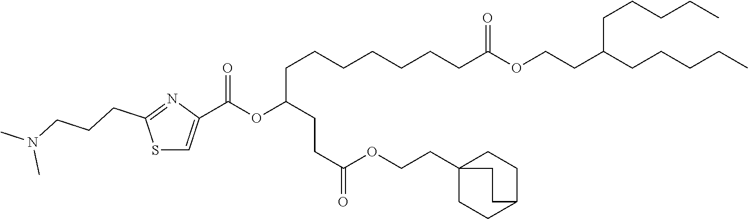 [SMILES: CCCCCC(CCCCC)CCOC(=O)CCCCCCCC(CCC(=O)OCCC12CCC(CC1)CC2)OC(=O)c1csc(CCCN(C)C)n1] |

### Series “AC”

In some embodiments, an LNP of the present disclosure comprises an ionizable lipid disclosed in PCT Application PCT/US2024/019990, which is incorporated by reference herein, in its entirety.

In some embodiments, ionizable lipids of the present disclosure have a structure of Formula (AC)

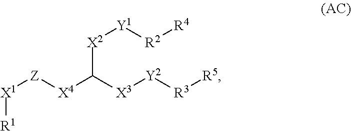

or a pharmaceutically acceptable salt thereof, wherein:
R¹ is selected from the group consisting of —NR₂,

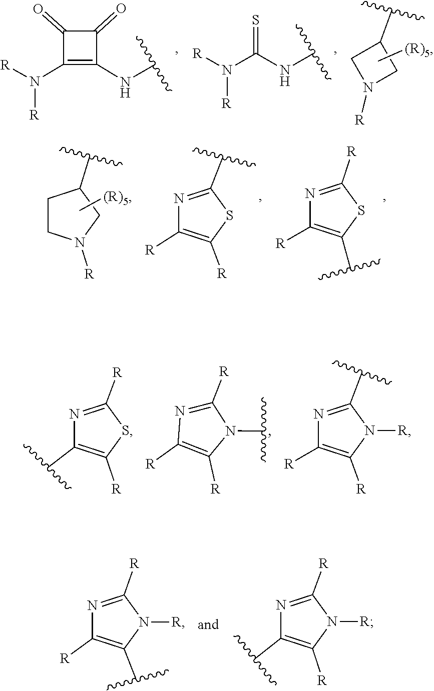

each R is independently —H or C₁-C₆ aliphatic;
X¹ is a bond or optionally substituted C₂-C₆ aliphatic;
Z is

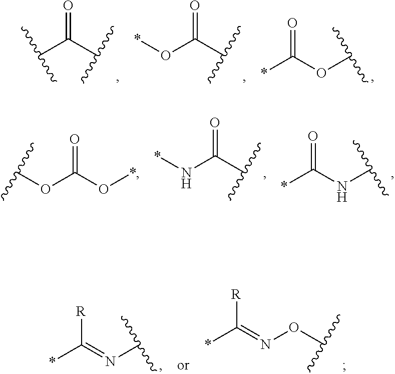

wherein the bond marked with an “*” is attached to X¹;
X² and X³ are each independently optionally substituted C₁-C₁₂ aliphatic;
X⁴ is a bond or C₂-C₆ aliphatic;
Y¹ and Y² are independently selected from the group consisting of

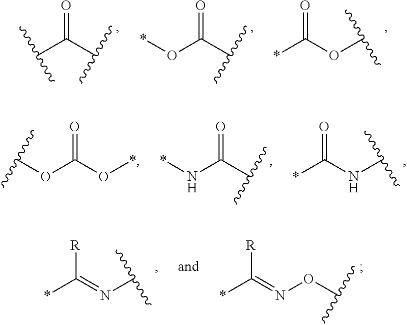

wherein the bond marked with an “*” is attached to X² for Y¹ or X³ for Y²;
R² is optionally substituted C₁-C₆ aliphatic;
R³ is optionally substituted C₁-C₆ aliphatic;
R⁴ is —CH(OR⁶)(OR⁷);
R⁵ is —CH(OR⁸)(OR⁹), —CH(R⁸)(R⁹), or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—; and
R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

### Additional Formulae

In certain embodiments, ionizable lipids of the present disclosure have a structure of Formula (AC), wherein the ionizable lipids of the present disclosure have a structure of Formula (AC-A), (AC-B), (AC-C), (AC-D), (AC-D1), (AC-D2), (AC-E), (AC-F), (AC-G), (AC-H), or (AC-I):

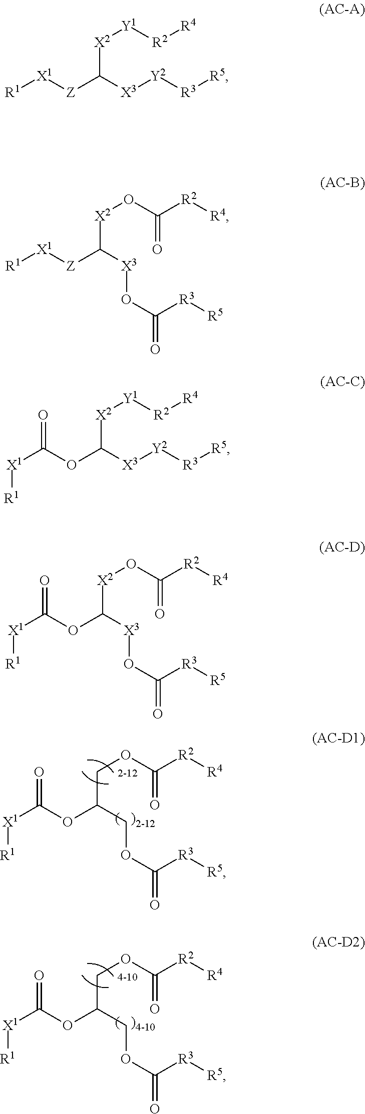

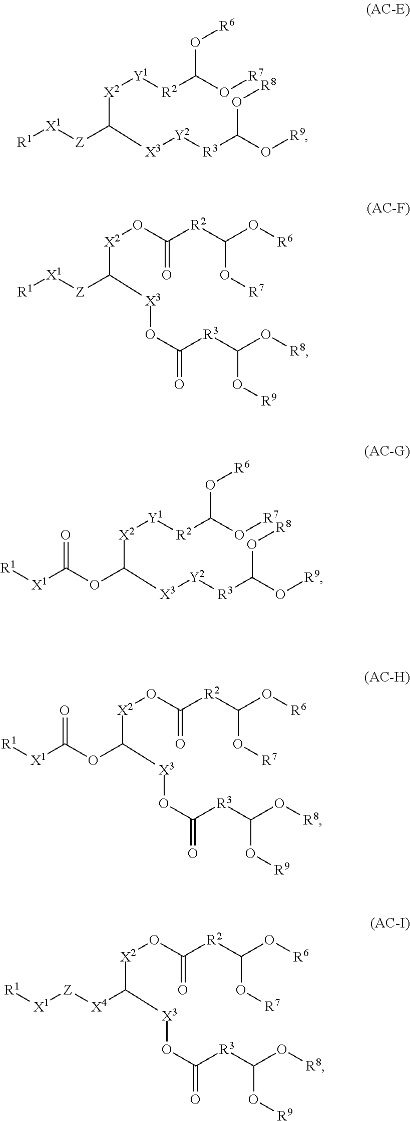

or a pharmaceutically acceptable salt thereof, wherein R¹, R, X¹, Z, X², X³, Y¹, Y², R², R³, R¹, R⁵, R⁶, R⁷, R⁸, and R⁹ are as described in Formula (AC) or as otherwise described in any embodiments below.

### R¹

In certain embodiments, R¹ is selected from the group consisting of —NR₂,

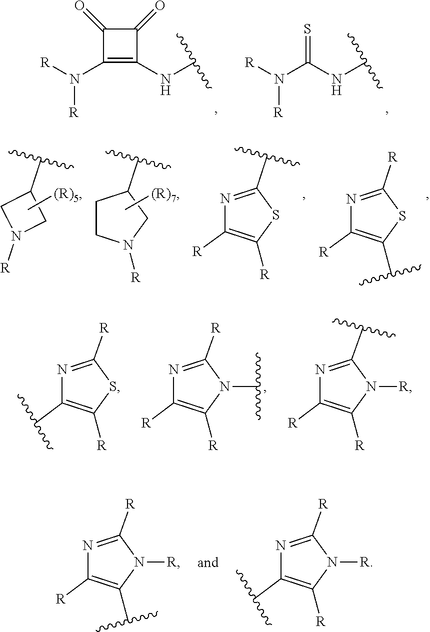

In certain embodiments, R¹ is selected from the group consisting of —NR₂,

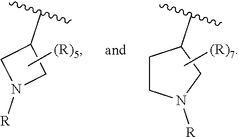

In certain embodiments, R¹ is —NR². In certain embodiments, R¹ is

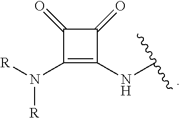

In certain embodiments, R¹ is

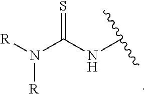

In certain embodiments, R¹ is

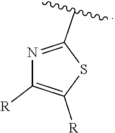

In certain embodiments, R¹ is

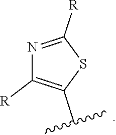

In certain embodiments, R¹ is

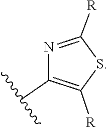

In certain embodiments, R¹ is

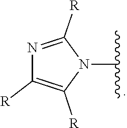

In certain embodiments, R¹ is

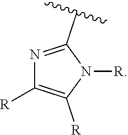

In certain embodiments, R¹ is

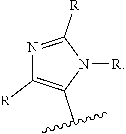

In certain embodiments, R¹ is

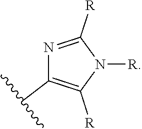

In certain embodiments, R¹ is

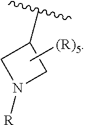

In certain embodiments, R¹ is

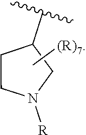

In certain embodiments, R¹ is selected from the group consisting of —N(Et)₂, —N(Me)(Et)

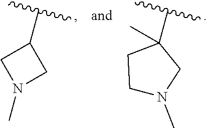

In certain embodiments, R¹ is —N(Et)₂. In certain embodiments, R¹ is —N(Me)₂. In certain embodiments, R¹ is —N(Me)(Et In certain embodiments, R¹ is —NH₂. In certain embodiments, R¹ is —N(nPr)₂. In certain embodiments, R¹ is —N(iPr)₂. In certain embodiments, R¹ is —N(Me)(Et). In certain embodiments, R¹ is

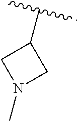 [SMILES: *C1CN(C)C1]

In certain embodiments, R¹ is

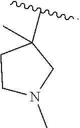 [SMILES: *C1(C)CCN(C)C1]

In certain embodiments, R¹ is

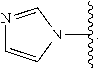 [SMILES: *n1ccnc1]

### X¹

In certain embodiments, X¹ is optionally substituted C₂-C₆ aliphatic. In certain embodiments, X¹ is optionally substituted C₂-C₆ alkylene. In certain embodiments, R² is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—. In certain embodiments, X¹ is a bond.

### Z

In certain embodiments, Lipids of the Disclosure have a structure of Formula (AC), (AC-A), (AC-B), (AC-E), (AC-F), or (AC-I), wherein Z is

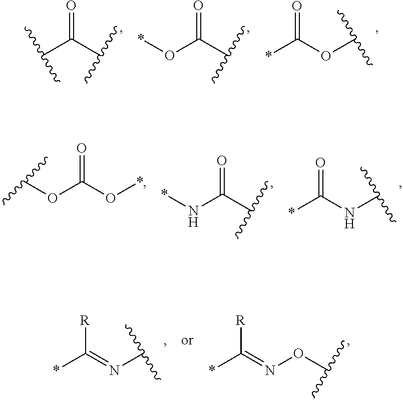

wherein the bond marked with an “*” is attached to X¹. In certain embodiments, Lipids of the Disclosure have a structure of Formula (AC), (AC-A), (AC-B), (AC-E), (AC-F), or (AC-I), Z is

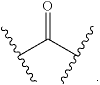 [SMILES: *C(*)=O]

In certain embodiments, Lipids of the Disclosure have a structure of Formula (AC), (AC-A), (AC-B), (AC-E), (AC-F), or (AC-I), Z is

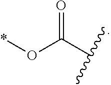 [SMILES: *OC(*)=O]

In certain embodiments, Lipids of the Disclosure have a structure of Formula (AC), (AC-A), (AC-B), (AC-E), (AC-F), or (AC-I), Z is

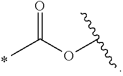 [SMILES: *OC(C)=O]

In certain embodiments, Lipids of the Disclosure have a structure of Formula (AC), (AC-A), (AC-B), (AC-E), (AC-F), or (AC-I), Z is

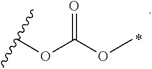

In certain embodiments, Lipids of the Disclosure have a structure of Formula (AC), (AC-A), (AC-B), (AC-E), (AC-F), or (AC-I), Z is

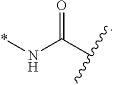 [SMILES: *NC(*)=O]

In certain embodiments, Lipids of the Disclosure have a structure of Formula (AC), (AC-A), (AC-B), (AC-E), (AC-F), or (AC-I), Y¹ is

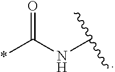 [SMILES: *NC(*)=O]

In certain embodiments, Lipids of the Disclosure have a structure of Formula (AC), (AC-A), (AC-B), (AC-E), (AC-F), or (AC-I), Z is

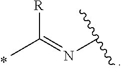

In certain embodiments, Lipids of the Disclosure have a structure of Formula (AC), (AC-A), (AC-B), (AC-E), (AC-F), or (AC-I), Z is

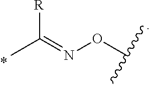

X² and X³

In certain embodiments, X² and X³ are each independently optionally substituted C₁-C₁₂ aliphatic. In certain embodiments, X² and X³ are the same. In certain embodiments, X² and X³ are different.

In certain embodiments, X² is an optionally substituted C₁-C₁₂ alkylene. In certain embodiments, X² is an optionally substituted C₁-C₁₂ alkenylene. I In certain embodiments, X² is an optionally substituted C₁-C₁₀ aliphatic. In certain embodiments, X² is an optionally substituted C₁-C₁₀ alkylene. In certain embodiments, X² is an optionally substituted C₁-C₁₀ alkenylene. In certain embodiments, X² is an optionally substituted C₁-C₈ aliphatic. In certain embodiments, X² is an optionally substituted C₁-C₈ alkylene. In certain embodiments, X² is an optionally substituted C₁-C₈ alkenylene. In certain embodiments, X² is an optionally substituted C₁-C₆ aliphatic. In certain embodiments, X² is an optionally substituted C₁-C₆ alkylene. In certain embodiments, X² is an optionally substituted C₁-C₆ alkenylene. In certain embodiments, X² is an optionally substituted C₂-C₁₂ aliphatic. In certain embodiments, X² is an optionally substituted C₂-C₁₂ alkylene. In certain embodiments, X² is an optionally substituted C₂-C₁₂ alkenylene. In certain embodiments, X² is an optionally substituted C₄-C₁₂ aliphatic. In certain embodiments, X² is an optionally substituted C₄-C₁₂ alkylene. In certain embodiments, X² is an optionally substituted C₄-C₁₂ alkenylene. In certain embodiments, X² is an optionally substituted C₄-C₁₀ aliphatic. In certain embodiments, X² is an optionally substituted C₄-C₁₀ alkylene. In certain embodiments, X² is an optionally substituted C₄-C₁₀ alkenylene. In certain embodiments, X² is an optionally substituted C₆-C₈ aliphatic. In certain embodiments, X² is an optionally substituted C₆-C₈ alkylene. In certain embodiments, X² is an optionally substituted C₆-C₈ alkenylene. In certain embodiments, X² is —(CH₂)—. In certain embodiments, X² is —(CH₂)₂—. In certain embodiments, X² is —(CH₂)₃—. In certain embodiments, X² is —(CH₂)₄—. In certain embodiments, X² is —(CH₂)₅—. In certain embodiments, X² is —(CH₂)₆—. In certain embodiments, X² is —(CH₂)₇—. In certain embodiments, X² is —(CH₂)₈—. In certain embodiments, X² is —(CH₂)₉—. In certain embodiments, X² is —(CH₂)₁₀—.

In certain embodiments, X³ is an optionally substituted C₁-C₁₂ alkylene. In certain embodiments, X³ is an optionally substituted C₁-C₁₂ alkenylene. In certain embodiments, X³ is an optionally substituted C₁-C₁₀ aliphatic. In certain embodiments, X³ is an optionally substituted C₁-C₁₀ alkylene. In certain embodiments, X³ is an optionally substituted C₁-C₁₀ alkenylene. In certain embodiments, X³ is an optionally substituted C₁-C₈ aliphatic. In certain embodiments, X³ is an optionally substituted C₁-C₈ alkylene. In certain embodiments, X³ is an optionally substituted C₁-C₈ alkenylene. In certain embodiments, X³ is an optionally substituted C₁-C₆ aliphatic. In certain embodiments, X³ is an optionally substituted C₁-C₆ alkylene. In certain embodiments, X³ is an optionally substituted C₁-C₆ alkenylene. In certain embodiments, X³ is an optionally substituted C₂-C₁₂ aliphatic. In certain embodiments, X³ is an optionally substituted C₂-C₁₂ alkylene. In certain embodiments, X³ is an optionally substituted C₂-C₁₂ alkenylene. In certain embodiments, X³ is an optionally substituted C₄-C₁₂ aliphatic. In certain embodiments, X³ is an optionally substituted C₄-C₁₂ alkylene. In certain embodiments, X³ is an optionally substituted C₄-C₁₂ alkenylene. In certain embodiments, X³ is an optionally substituted C₄-C₁₀ aliphatic. In certain embodiments, X³ is an optionally substituted C₄-C₁₀ alkylene. In certain embodiments, X³ is an optionally substituted C₄-C₁₀ alkenylene. In certain embodiments, X³ is an optionally substituted C₆-C₈ aliphatic. In certain embodiments, X³ is an optionally substituted C₆-C₈ alkylene. In certain embodiments, X³ is an optionally substituted C₆-C₈ alkenylene. In certain embodiments, X³ is —(CH₂)—. In certain embodiments, X³ is —(CH₂)₂—. In certain embodiments, X³ is —(CH₂)₃—. In certain embodiments, X³ is —(CH₂)₄—. In certain embodiments, X³ is —(CH₂)₅—. In certain embodiments, X³ is —(CH₂)₆—. In certain embodiments, X³ is —(CH₂)₇—. In certain embodiments, X³ is —(CH₂)₈—. In certain embodiments, X³ is —(CH₂)₉—. In certain embodiments, X³ is —(CH₂)₁₀—.

In certain embodiments, X² and X³ are both —(CH₂)₈—. In certain embodiments, X² and X³ are both —(CH₂)₆—.

### X⁴

In certain embodiments, Lipids of the Disclosure have a structure of Formula (AC) or (AC-I), wherein X⁴ is a bond or C₂-C₆ aliphatic. In certain embodiments, Lipids of the Disclosure have a structure of Formula (AC) or (AC-I), wherein X⁴ is a bond. In certain embodiments, Lipids of the Disclosure have a structure of Formula (AC) or (AC-I), wherein X⁴ is C₂-C₆ aliphatic. In certain embodiments, Lipids of the Disclosure have a structure of Formula (AC) or (AC-I), wherein X⁴ is C₂ aliphatic. In certain embodiments, Lipids of the Disclosure have a structure of Formula (AC) or (AC-I), wherein X⁴ is C₃ aliphatic. In certain embodiments, Lipids of the Disclosure have a structure of Formula (AC) or (AC-I), wherein X⁴ is C₄ aliphatic. In certain embodiments, Lipids of the Disclosure have a structure of Formula (AC) or (AC-I), wherein X⁴ is C₅ aliphatic. In certain embodiments, Lipids of the Disclosure have a structure of Formula (AC) or (AC-I), wherein X⁴ is C₆ aliphatic.

Y¹ and Y²

In certain embodiments, Y¹ and Y² are each independently

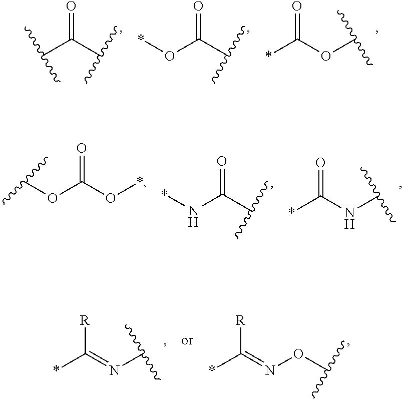

wherein the bond marked with an “*” is attached to X² for Y¹ or X³ for Y². In certain embodiments, Y¹ and Y² are the same. In certain embodiments, Y¹ and Y² are different.

In certain embodiments, Y¹ is

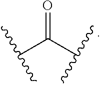 [SMILES: *C(*)=O]

In certain embodiments, Y¹ is

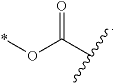 [SMILES: *OC(*)=O]

In certain embodiments, Y¹ is

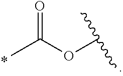 [SMILES: *OC(C)=O]

In certain embodiments, Y¹ is

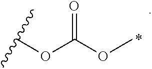

In certain embodiments, Y¹ is

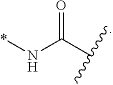 [SMILES: *NC(*)=O]

In certain embodiments, Y¹ is

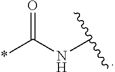 [SMILES: *NC(*)=O]

In certain embodiments, Y¹ is

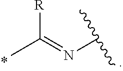

In certain embodiments, Y¹ is

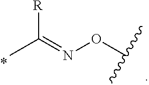

In certain embodiments, Y² is

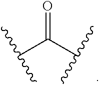 [SMILES: *C(*)=O]

In certain embodiments, Y² is

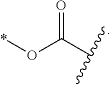 [SMILES: *OC(*)=O]

In certain embodiments, Y² is

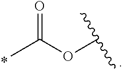 [SMILES: *OC(C)=O]

In certain embodiments, Y² is

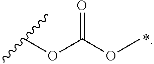

In certain embodiments, Y² is

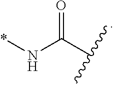 [SMILES: *NC(*)=O]

In certain embodiments, Y² is

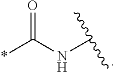 [SMILES: *NC(*)=O]

In certain embodiments, Y² is

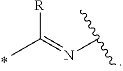

In certain embodiments, Y² is

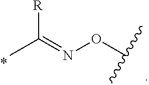

In certain embodiments, Y¹ and Y² are both

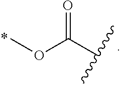 [SMILES: *C(=O)OC]

In certain embodiments, Y¹ and Y² are both

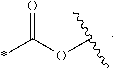 [SMILES: *OC(*)=O]

### R²

In certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

In certain embodiments, R³ is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different.

### R⁴

In certain embodiments, R⁴ is —CH(OR⁶)(OR⁷).

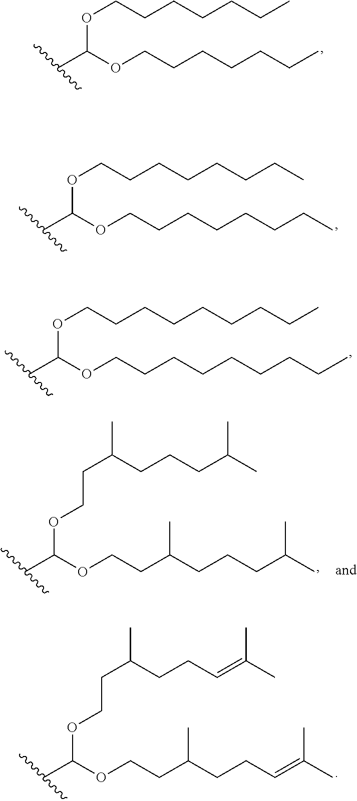 [SMILES: *C(OCCCCCCC)OCCCCCCC ; *C(OCCCCCCCC)OCCCCCCCC ; *C(OCCCCCCCCC)OCCCCCCCCC ; *C(OCCC(C)CCCC(C)C)OCCC(C)CCCC(C)C ; *C(OCCC(C)CCC=C(C)C)OCCC(C)CCC=C(C)C]

In certain embodiments, R⁴ is selected from

### R⁵

In certain embodiments, R⁵ is optionally substituted C₁-C₁₄ aliphatic, —CHI (OR⁸)(OR⁹); or —CH(R⁸)(R⁹). In certain embodiments, R⁵ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹).

In certain embodiments, one of the methylene linkages of R⁵ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

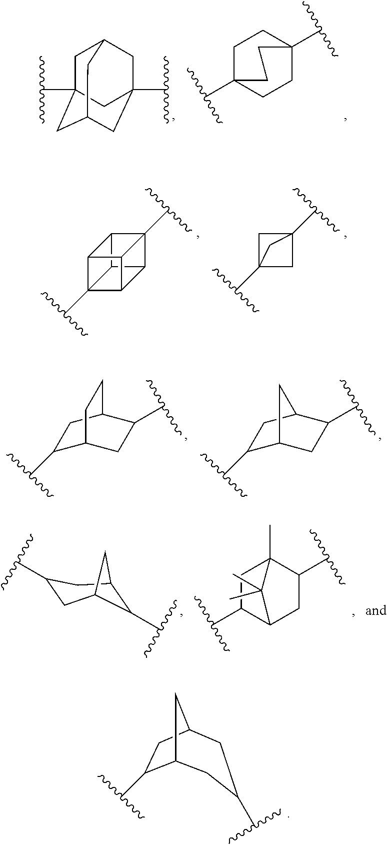

In certain embodiments, R⁴ and R⁵ are the same. In certain embodiments, R⁴ and R⁵ are different.

In certain embodiments, R⁵ is selected from

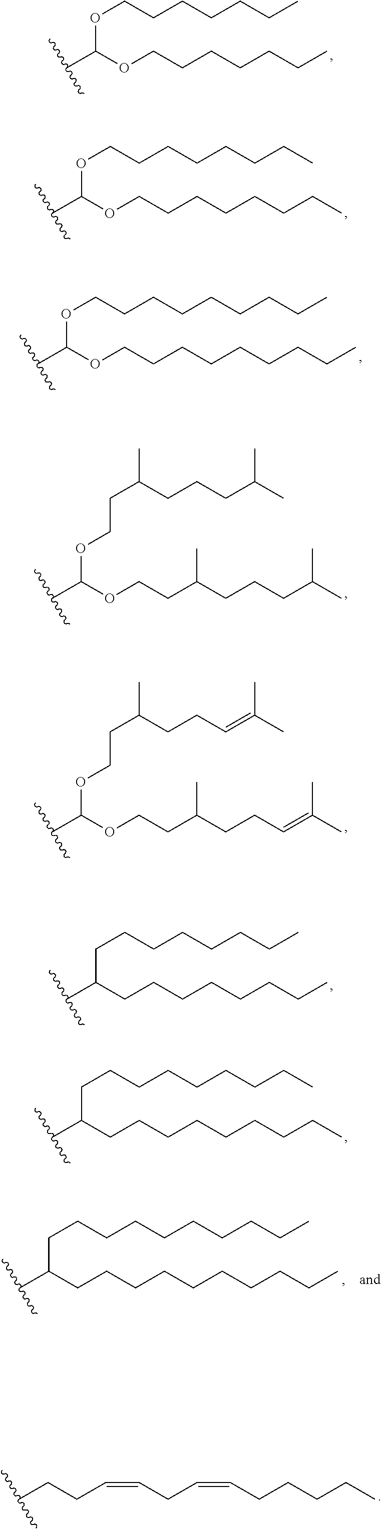

In certain embodiments, R⁵ is selected from

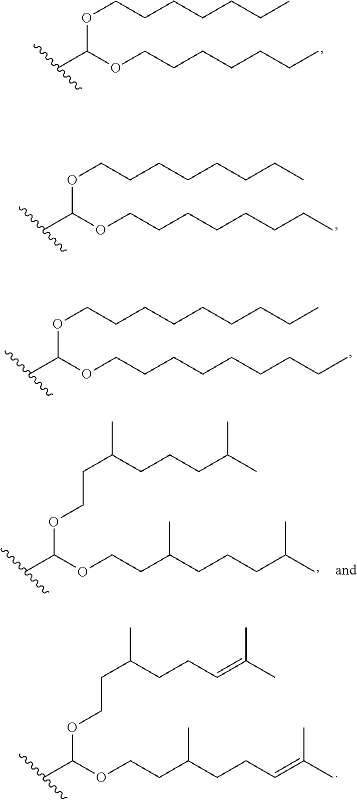 [SMILES: *C(OCCCCCCC)OCCCCCCC ; *C(OCCCCCCCC)OCCCCCCCC ; *C(OCCCCCCCCC)OCCCCCCCCC ; *C(OCCC(C)CCCC(C)C)OCCC(C)CCCC(C)C ; *C(OCCC(C)CCC=C(C)C)OCCC(C)CCC=C(C)C]

R⁶ and R⁷

In certain embodiments, R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkylene. In certain embodiments, R⁶ is optionally substituted C₁-C₁₄ branched alkylene. In certain embodiments, R⁶ is optionally substituted C₁-C₁₄ straight chain alkylene. In certain embodiments, R⁶ is optionally substituted C₁-C₁₄ alkenylene. In certain embodiments, R⁶ is optionally substituted C₁-C₁₄ branched alkenylene. In certain embodiments, R⁶ is optionally substituted C₁-C₁₄ straight chain alkenylene. In certain embodiments, R⁶ is optionally substituted C₆-C₁₀ alkylene. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)—CH₃. In certain embodiments, R⁶ is optionally substituted —(CH₂)₈CH₃. In certain embodiments, R⁶ is optionally substituted —(CH₂)₉CH₃.

In certain embodiments, one of the methylene linkages of R⁶ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

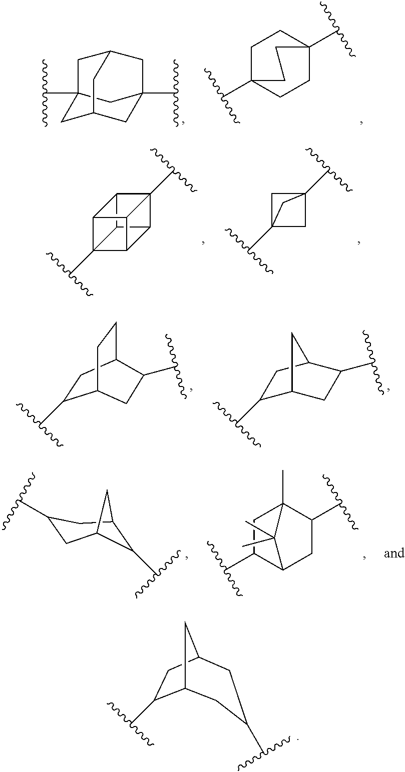

In certain embodiments, R⁷ is optionally substituted C₁-C₁₄ aliphatic. In certain embodiments, R⁷ is optionally substituted C₁-C₁₄ alkylene. In certain embodiments, R⁷ is optionally substituted C₁-C₁₄ branched alkylene. In certain embodiments, R⁷ is optionally substituted C₁-C₁₄ straight chain alkylene. In certain embodiments, R⁷ is optionally substituted C₁-C₁₄ alkenylene. In certain embodiments, R⁷ is optionally substituted C₁-C₁₄ branched alkenylene. In certain embodiments, R⁷ is optionally substituted C₁-C₁₄ straight chain alkenylene. In certain embodiments, R⁷ is optionally substituted C₆-C₁₀ alkylene. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁶ is optionally substituted —(CH₂)₉CH₃.

In certain embodiments, one of the methylene linkages of R⁷ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

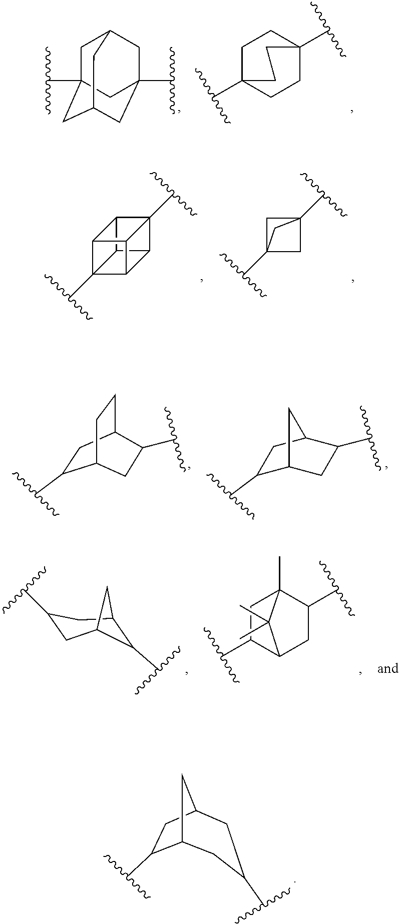

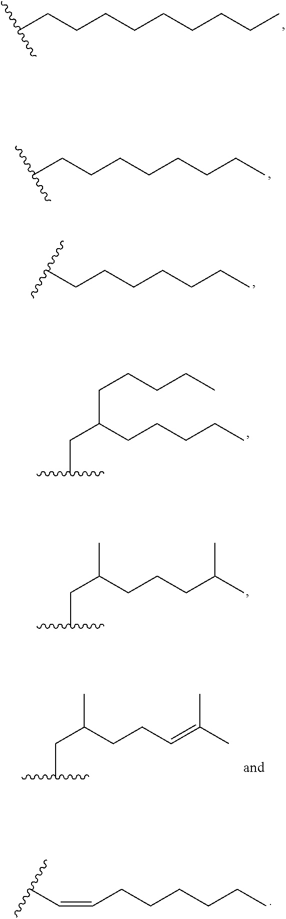

R⁸ and R⁹

In certain embodiments, R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is optionally substituted C₁-C₁₄ aliphatic. In certain embodiments, R⁸ is optionally substituted C₁-C₁₄ alkylene. In certain embodiments, R⁸ is optionally substituted C₁-C₁₄ branched alkylene. In certain embodiments, R⁸ is optionally substituted C₁-C₁₄ straight chain alkylene. In certain embodiments, R⁸ is optionally substituted C₁-C₁₄ alkenylene. In certain embodiments, R⁸ is optionally substituted C₁-C₁₄ branched alkenylene. In certain embodiments, R⁸ is optionally substituted C₁-C₁₄ straight chain alkenylene. In certain embodiments, R⁸ is optionally substituted C₆-C₁₀ alkylene. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, one of the methylene linkages of R⁸ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

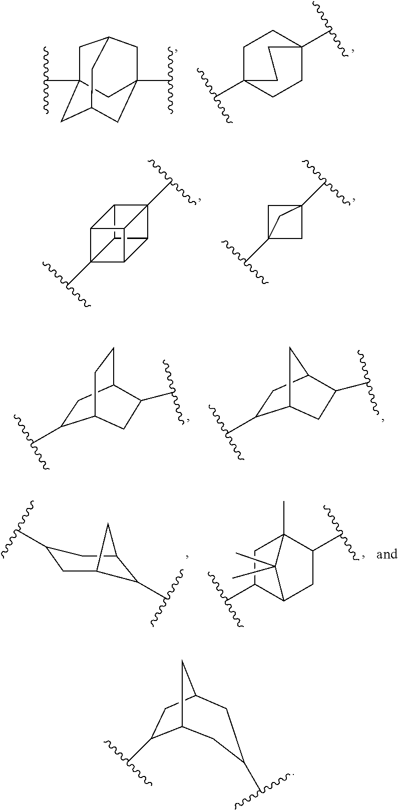

In certain embodiments, R⁹ is optionally substituted C₁-C₁₄ aliphatic. In certain embodiments, R⁹ is optionally substituted C₁-C₁₄ alkylene. In certain embodiments, R⁹ is optionally substituted C₁-C₁₄ branched alkylene. In certain embodiments, R⁹ is optionally substituted C₁-C₁₄ straight chain alkylene. In certain embodiments, R⁹ is optionally substituted C₁-C₁₄ alkenylene. In certain embodiments, R⁹ is optionally substituted C₁-C₁₄ branched alkenylene. In certain embodiments, R⁹ is optionally substituted C₁-C₁₄ straight chain alkenylene. In certain embodiments, R⁹ is optionally substituted C₆-C₁₀ alkylene. In certain embodiments, R⁹ is optionally substituted —(CH₂)₅CH₃. In certain embodiments, R⁹ is optionally substituted —(CH₂)₆CH₃. In certain embodiments, Rois optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, one of the methylene linkages of R⁹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

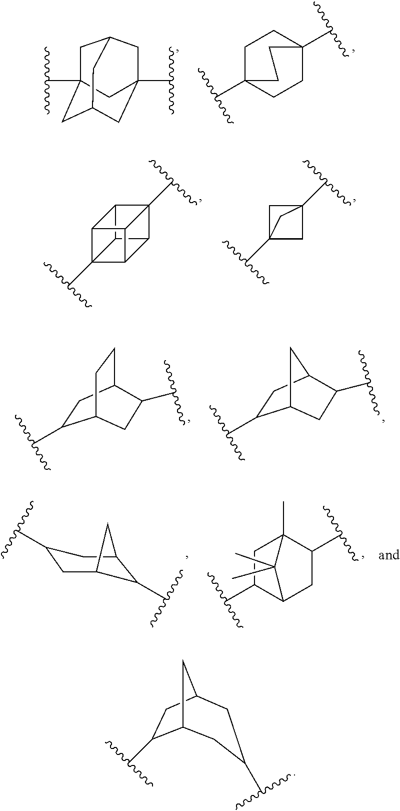

In certain embodiments, R⁸ and R⁹ are selected from

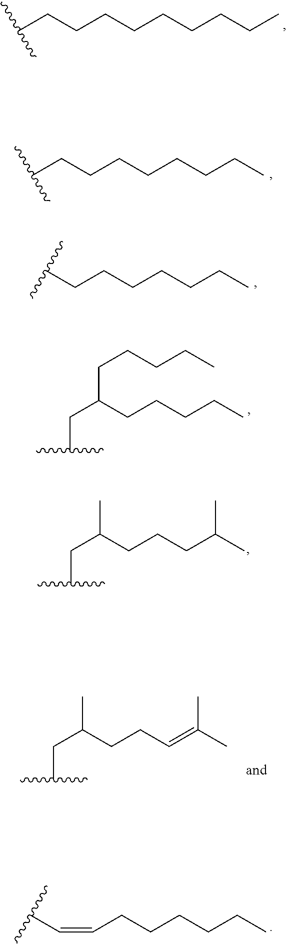

In some embodiments, Lipids of the Present Disclosure are selected from any lipid in Table (VIII) below or a pharmaceutically acceptable salt thereof:

TABLE (VIII)
Non-Limiting Examples of Ionizable Lipids of the Present Disclosure

| Cmpd | Structure |
|---|---|
| AC-1 | 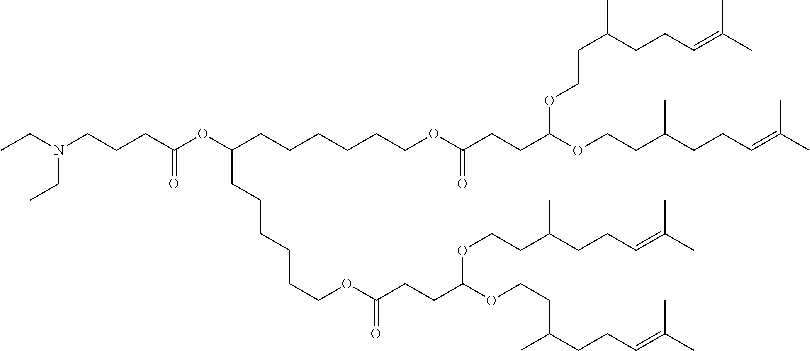 [SMILES: CCN(CC)CCCC(=O)OC(CCCCCCOC(=O)CCC(OCCC(C)CCC=C(C)C)OCCC(C)CCC=C(C)C)CCCCCCOC(=O)CCC(OCCC(C)CCC=C(C)C)OCCC(C)CCC=C(C)C] |
| AC-2 | 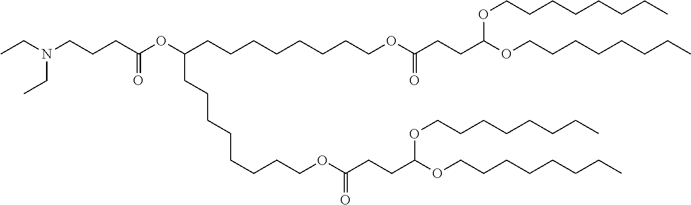 [SMILES: CCCCCCCCOC(CCC(=O)OCCCCCCCCC(CCCCCCCCOC(=O)CCC(OCCCCCCCC)OCCCCCCCC)OC(=O)CCCN(CC)CC)OCCCCCCCC] |
| AC-3 | 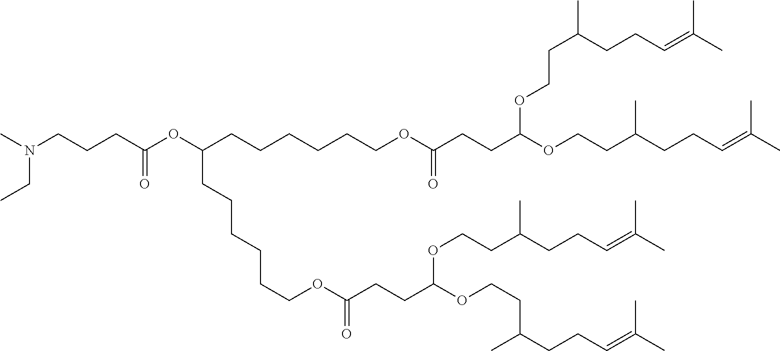 [SMILES: CCN(C)CCCC(=O)OC(CCCCCCOC(=O)CCC(OCCC(C)CCC=C(C)C)OCCC(C)CCC=C(C)C)CCCCCCOC(=O)CCC(OCCC(C)CCC=C(C)C)OCCC(C)CCC=C(C)C] |
| AC-4 | 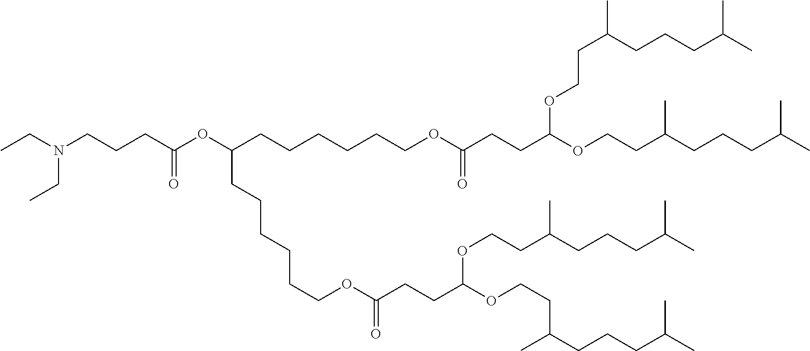 [SMILES: CCN(CC)CCCC(=O)OC(CCCCCCOC(=O)CCC(OCCC(C)CCCC(C)C)OCCC(C)CCCC(C)C)CCCCCCOC(=O)CCC(OCCC(C)CCCC(C)C)OCCC(C)CCCC(C)C] |
| AC-5 | 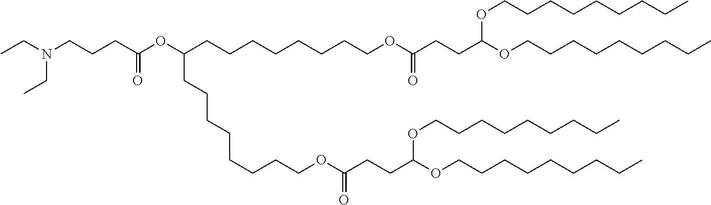 [SMILES: CCCCCCCCCOC(CCC(=O)OCCCCCCCCC(CCCCCCCCOC(=O)CCC(OCCCCCCCCC)OCCCCCCCCC)OC(=O)CCCN(CC)CC)OCCCCCCCCC] |
| AC-6 | 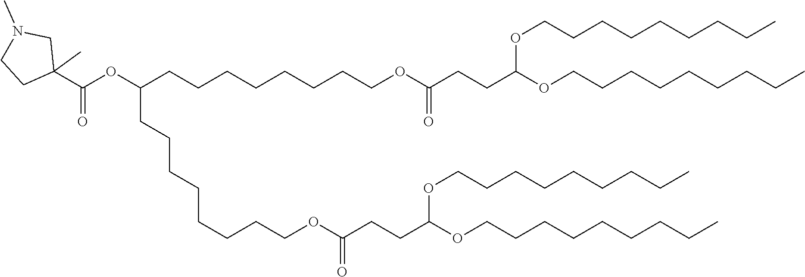 [SMILES: CCCCCCCCCOC(CCC(=O)OCCCCCCCCC(CCCCCCCCOC(=O)CCC(OCCCCCCCCC)OCCCCCCCCC)OC(=O)C1(C)CCN(C)C1)OCCCCCCCCC] |
| AC-7 | 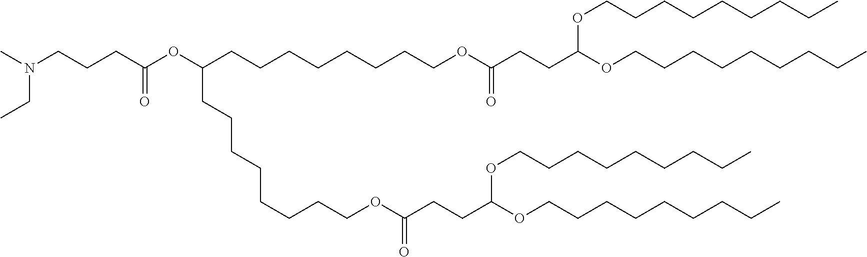 [SMILES: CCCCCCCCCOC(CCC(=O)OCCCCCCCCC(CCCCCCCCOC(=O)CCC(OCCCCCCCCC)OCCCCCCCCC)OC(=O)CCCN(C)CC)OCCCCCCCCC] |
| AC-8 | 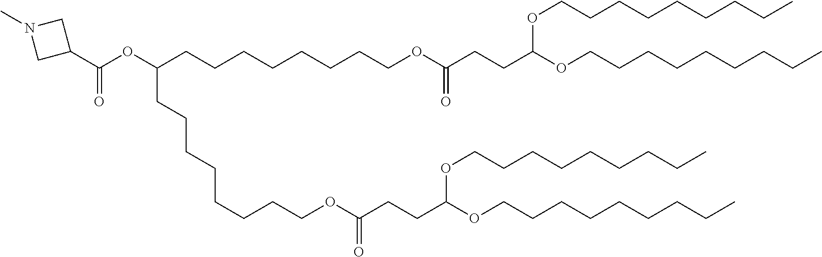 [SMILES: CCCCCCCCCOC(CCC(=O)OCCCCCCCCC(CCCCCCCCOC(=O)CCC(OCCCCCCCCC)OCCCCCCCCC)OC(=O)C1CN(C)C1)OCCCCCCCCC] |
| AC-9 | 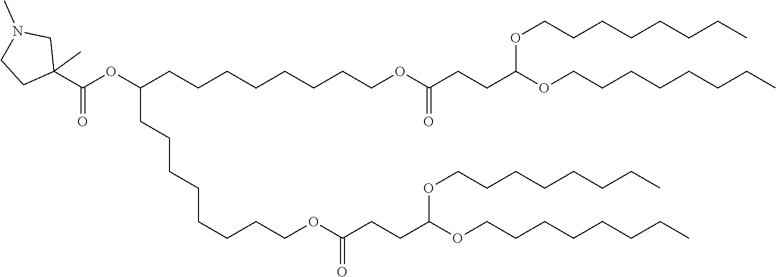 [SMILES: CCCCCCCCOC(CCC(=O)OCCCCCCCCC(CCCCCCCCOC(=O)CCC(OCCCCCCCC)OCCCCCCCC)OC(=O)C1(C)CCN(C)C1)OCCCCCCCC] |
| AC- 10 | 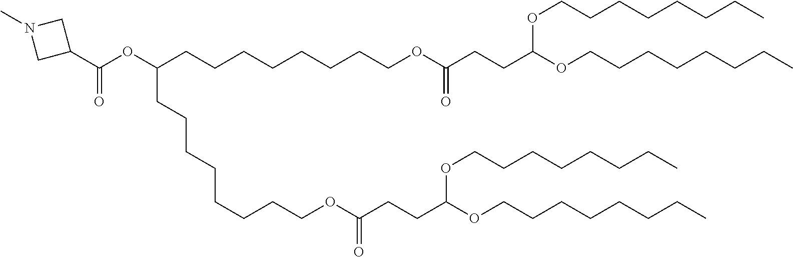 [SMILES: CCCCCCCCOC(CCC(=O)OCCCCCCCCC(CCCCCCCCOC(=O)CCC(OCCCCCCCC)OCCCCCCCC)OC(=O)C1CN(C)C1)OCCCCCCCC] |

### Series “CO”

In some embodiments, an LNP of the present disclosure comprises an ionizable lipid disclosed in PCT Application PCT/US2024/019990, which is incorporated by reference herein, in its entirety.

The present disclosure provides compound of Formula (CO):

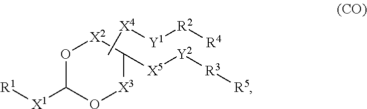

or a pharmaceutically acceptable salt thereof,
wherein:
R¹ is selected from the group consisting of —NR₂,

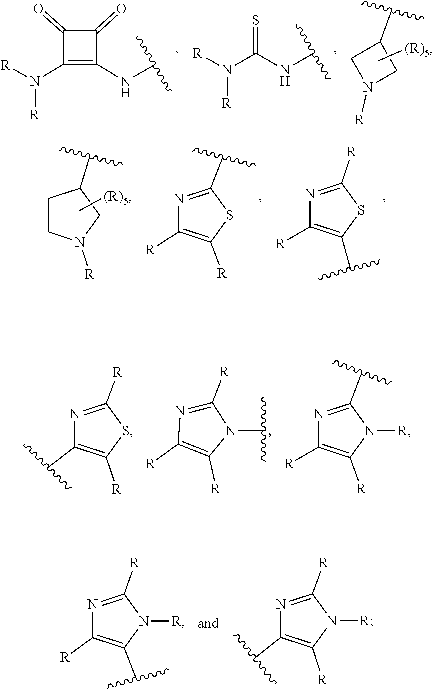

each R is independently —H or C₁-C₆ aliphatic;
X¹ is optionally substituted C₂-C₆ aliphatic, wherein one or more methylene linkages are each optionally and independently replaced with —O—, —NH—, —S—, —SS—, —C(O)—, —OC(O)O—, —OC(O)—, —NHC(O)— or —C(O)O—;
X² is selected from the group consisting of a bond, —CH₂— and —CH₂CH₂—;
X³ is selected from the group consisting of a bond, —CH₂— and —CH₂CH₂—;
X⁴ and X⁵ are each independently optionally substituted C₁-C₁₀ aliphatic;
Y¹ and Y² are each independently

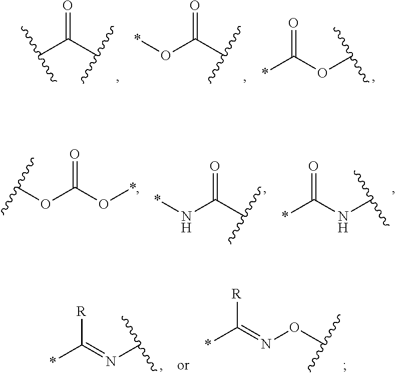

wherein the bond marked with an “*” is attached to X⁴ or X⁵;
R² is optionally substituted C₁-C₆ aliphatic;
R³ is optionally substituted C₁-C₆ aliphatic;
R⁴ is —CH(OR⁶)(OR⁷); —CH(SR⁶)(SR⁷); —CH(R⁶)(R⁷); or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁵ is —CH(OR⁸)(OR⁹); —CH(SR⁸)(SR⁹); —CH(R⁸)(R⁹) or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—;
R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—; and
R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

### Additional Formulae

In certain embodiments, the compound of Formula (CO) is a compound of any of the below Formulae:

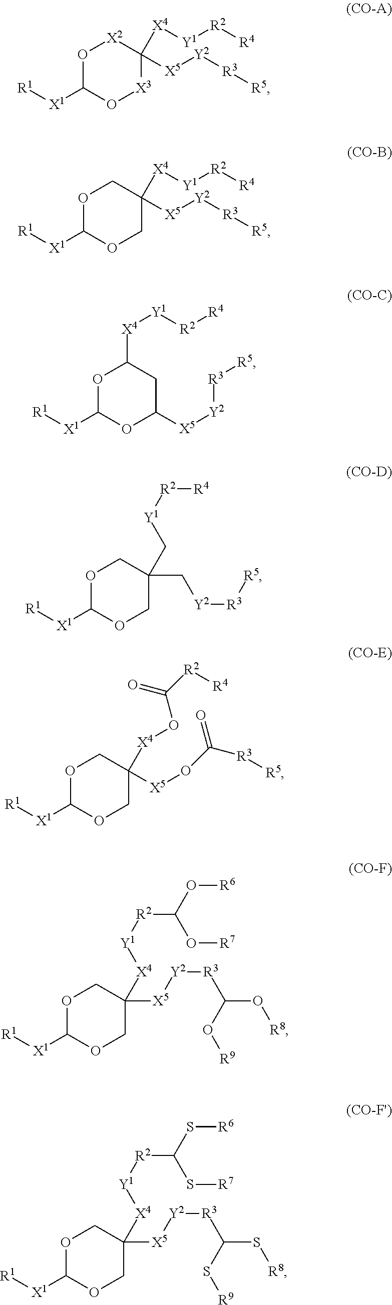

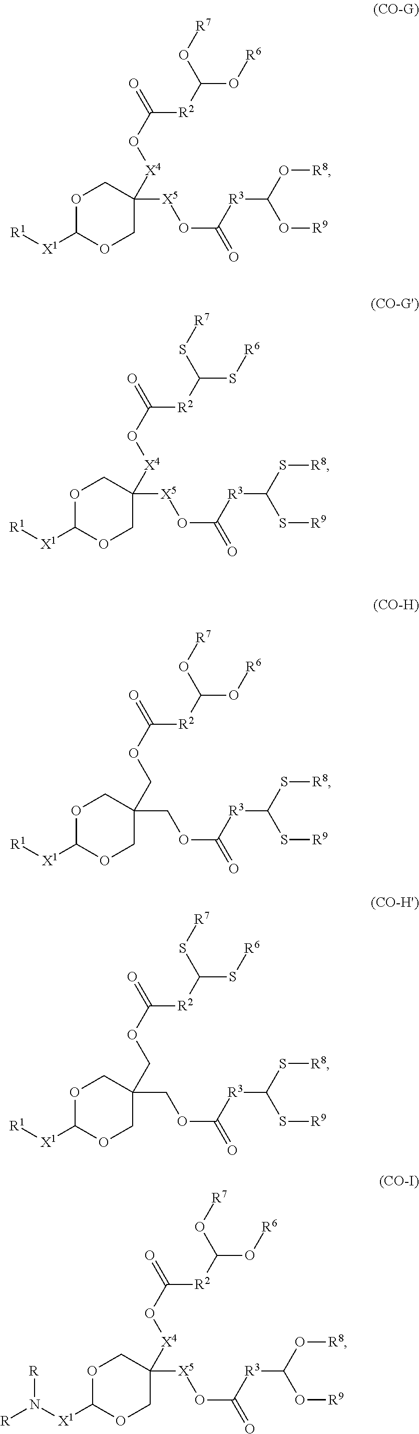

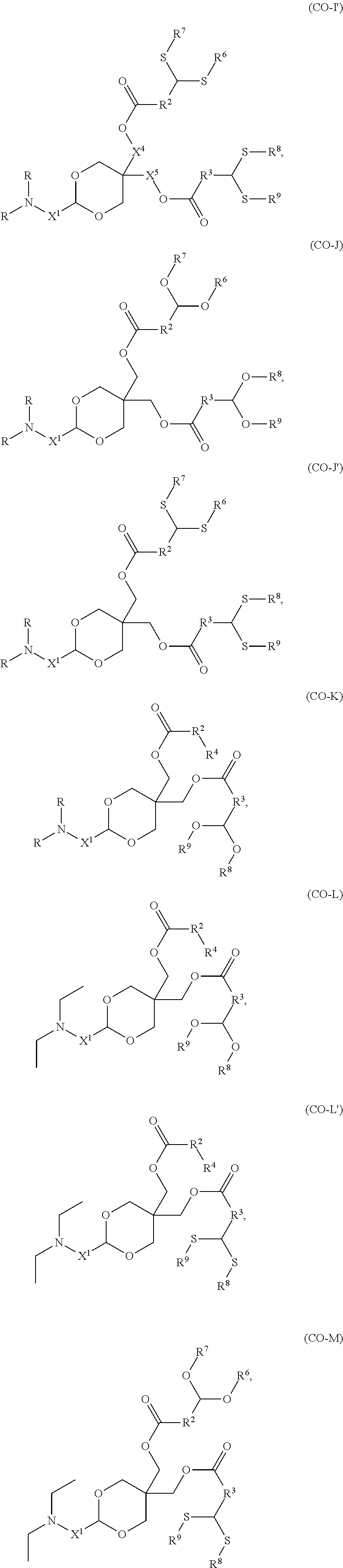

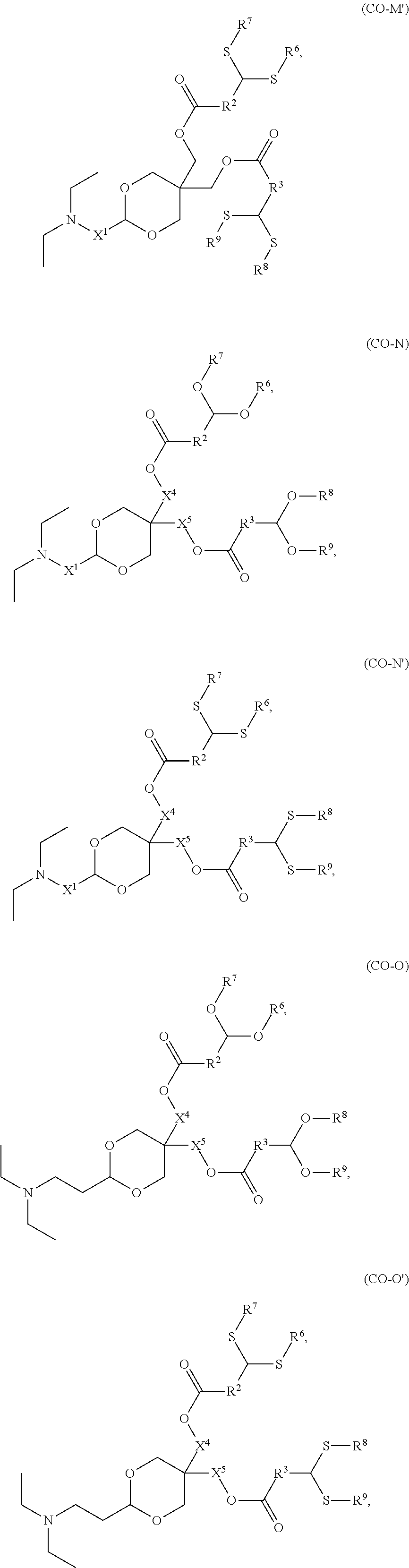

or a pharmaceutically acceptable salt thereof, wherein R¹, R, X¹, X², X³, X⁴, X⁵, Y¹, Y², R², R³, R⁴, R⁵, R⁶, R⁷, R⁸, and R⁹ are as described in Formula (CO) or as otherwise described in any embodiments below.

### R¹

As disclosed in Formula (CO), in certain embodiments, R¹ is selected from the group consisting of —NR₂,

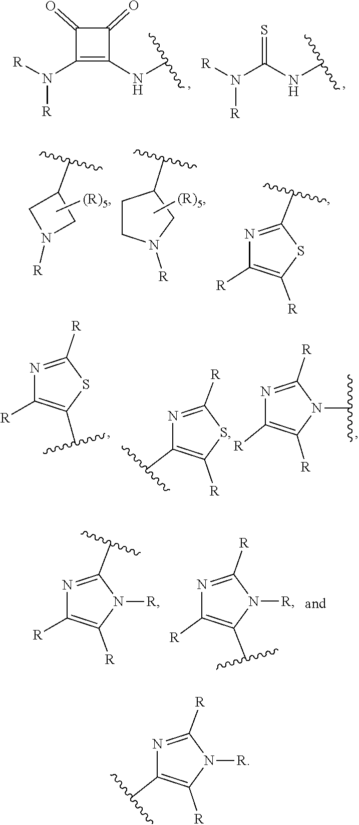

In certain embodiments, R¹ is —NR². In certain embodiments, R¹ is

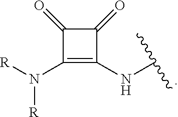

In certain embodiments, R¹ is

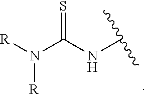

In certain embodiments, R¹ is

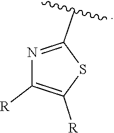

In certain embodiments, R¹ is

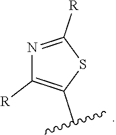

In certain embodiments, R¹ is

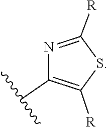

In certain embodiments, R¹ is

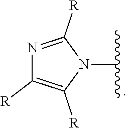

In certain embodiments, R¹ is

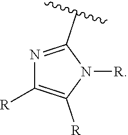

In certain embodiments, R¹ is

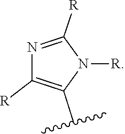

In certain embodiments, R¹ is

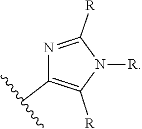

In certain embodiments, R¹ is

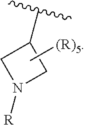

In certain embodiments, R¹ is

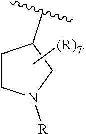

In certain embodiments, R¹ is —N(Et)₂. In certain embodiments, R¹ is —N(Me)₂. In certain embodiments, R¹ is —NH₂. In certain embodiments, R¹ is —N(nPr)₂. In certain embodiments, R¹ is —N(iPr)₂. In certain embodiments, R¹ is —N(Me)(Et). In certain embodiments, R¹ is

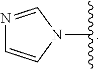 [SMILES: *n1ccnc1]

### X¹

As disclosed in Formula (CO), in certain embodiments, X¹ is optionally substituted C₂-C₆ aliphatic, wherein one or more methylene linkages are each optionally and independently replaced with —O—, —NH—, —S—, —SS—, —C(O)—, —OC(O)O—, —NHC(O)— or —C(O)O—. In certain embodiments, X¹ is optionally substituted C₂-C₆ aliphatic. In certain embodiments, X¹ is optionally substituted C₂-C₆ alkylene. In certain embodiments, X¹ is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X′ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

### X²

As disclosed in Formula (CO), in certain embodiments, X² is selected from the group consisting of a bond, —CH₂— and —CH₂CH₂—. In certain embodiments, X² is a bond. In certain embodiments, X² is —CH₂—. In certain embodiments, X² is —CH₂CH₂—.

### X³

As disclosed in Formula (CO), in certain embodiments, X³ is selected from the group consisting of a bond, —CH₂— and —CH₂CH₂—. In certain embodiments, X³ is a bond. In certain embodiments, X³ is —CH₂—. In certain embodiments, X³ is —CH₂CH₂—. In certain embodiments, both X² and X³ are —CH₂—. In certain embodiments, both X² and X³ are —CH₂CH₂—. In certain embodiments, X² is a bond and X³ is —CH₂—. In certain embodiments, X² is a bond and X³ is —CH₂CH₂—. In certain embodiments, X³ is a bond and X² is —CH₂—. In certain embodiments, X³ is a bond and X² is —CH₂CH₂—.

X⁴ and X⁵

As disclosed in Formula (CO), in certain embodiments, X⁴ and X⁵ are each independently optionally substituted C₁-C₁₀ aliphatic. In certain embodiments, X⁴ and X⁵ are the same. In certain embodiments, X⁴ and X⁵ are different.

In certain embodiments, X⁴ is an optionally substituted C₁-C₁₀ alkylene. In certain embodiments, X⁴ is an optionally substituted C₁-C₁₀ alkenylene. In certain embodiments, X⁴ is an optionally substituted C₁-C₆ alkylene. In certain embodiments, X⁴ is an optionally substituted C₁-C₆ alkenylene. In certain embodiments, X⁴ is —(CH₂)—. In certain embodiments, X⁴ is —(CH₂)₂—. In certain embodiments, X⁴ is —(CH₂)₃—. In certain embodiments, X⁴ is —(CH₂)₄—. In certain embodiments, X⁴ is —(CH₂)₅—. In certain embodiments, X⁴ is —(CH₂)₆—.

In certain embodiments, X⁵ is an optionally substituted C₁-C₁₀ alkylene. In certain embodiments, X⁵ is an optionally substituted C₁-C₁₀ alkenylene. In certain embodiments, X⁵ is an optionally substituted C₁-C₆ alkylene. In certain embodiments, X⁵ is an optionally substituted C₁-C₆ alkenylene. In certain embodiments, X⁵ is —(CH₂)—. In certain embodiments, X⁵ is —(CH₂)₂—. In certain embodiments, X⁵ is —(CH₂)₃—. In certain embodiments, X⁵ is —(CH₂)₄—. In certain embodiments, X⁵ is —(CH₂)₅—. In certain embodiments, X⁵ is —(CH₂)₆—.

In certain embodiments, X⁴ and X⁵ are both —(CH₂)—. In certain embodiments, X⁴ and X⁵ are both —(CH₂)₂—.

Y¹ and Y²

As disclosed in Formula (CO), in certain embodiments, Y¹ and Y² are each independently

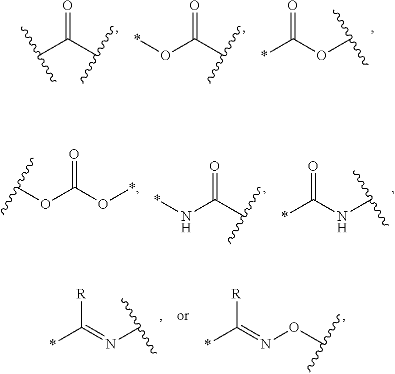

wherein the bond marked with an “*” is attached to X⁴ or X⁵. In certain embodiments, Y¹ and Y² are the same. In certain embodiments, Y¹ and Y² are different.

In certain embodiments, Y¹ is

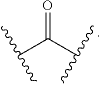 [SMILES: *C(*)=O]

In certain embodiments, Y¹ is

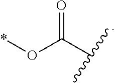 [SMILES: *OC(*)=O]

In certain embodiments, Y¹ is

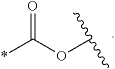 [SMILES: *OC(*)=O]

In certain embodiments, Y¹ is

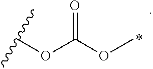

In certain embodiments, Y¹ is

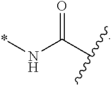 [SMILES: *NC(*)=O ; "."]

In certain embodiments, Y¹ is

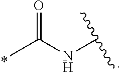

In certain embodiments, Y¹ is

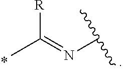

In certain embodiments, Y¹ is

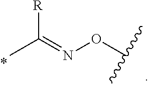

In certain embodiments, Y² is

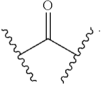 [SMILES: *C(*)=O]

In certain embodiments, Y² is

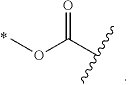 [SMILES: *C(=O)OC]

In certain embodiments, Y² is

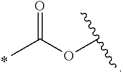 [SMILES: *OC(C)=O]

In certain embodiments, Y² is

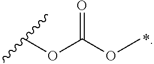

In certain embodiments, Y² is

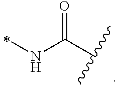

In certain embodiments, Y² is

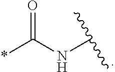 [SMILES: *NC(*)=O]

In certain embodiments, Y² is

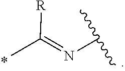

In certain embodiments, Y² is

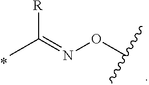

In certain embodiments, Y¹ and Y² are both

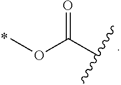 [SMILES: *C(=O)OC]

In certain embodiments, Y¹ and Y² are both

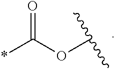 [SMILES: *OC(*)=O]

### R²

As disclosed in Formula (CO), in certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

As disclosed in Formula (CO), in certain embodiments, R³ is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different. In certain embodiments, R² and R³ are both —(CH₂)₂—.

### R⁴

As disclosed in Formula (CO), in certain embodiments, R⁴ is —CH(OR⁶)(OR⁷); —CH(SR⁶)(SR⁷); —CH(R⁶)(R⁷); or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic. In certain embodiments, R⁴ is —CH(OR⁶)(OR⁷). In certain embodiments, R⁴ is —CH(R⁶)(R⁷). In certain embodiments, R⁴ is —CH(SR⁶)(SR⁷).

In certain embodiments, R⁴ is selected from

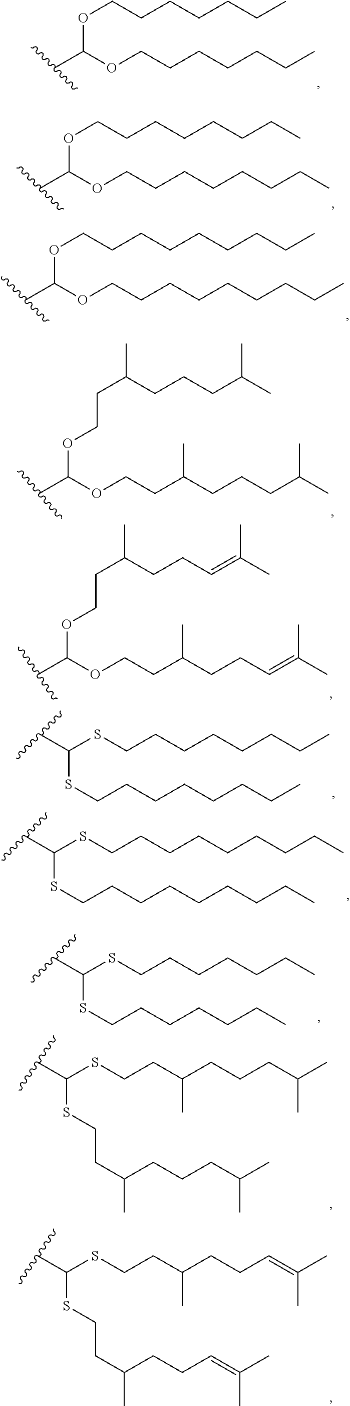

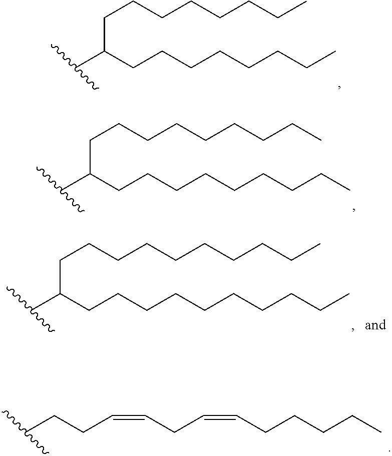 [SMILES: *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

In certain embodiments, R⁴ is selected from

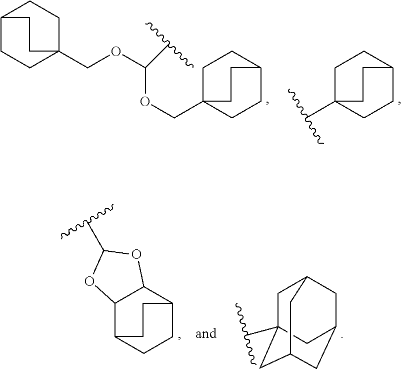 [SMILES: *C(OCC12CCC(CC1)CC2)OCC12CCC(CC1)CC2 ; *C12CCC(CC1)CC2 ; *C1OC2C3CCC(CC3)C2O1 ; *C12CC3CC(CC(C3)C1)C2]

### R⁵

As disclosed in Formula (CO), in certain embodiments, R⁵ is —CH(OR⁸)(OR⁹); —CH(SR⁸)(SR⁹); —CH(R⁸)(R⁹) or optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SR⁸)(SR⁹).

In certain embodiments, R⁴ and R⁵ are the same. In certain embodiments, R⁴ and R⁵ are different.

In certain embodiments, R⁵ is selected from

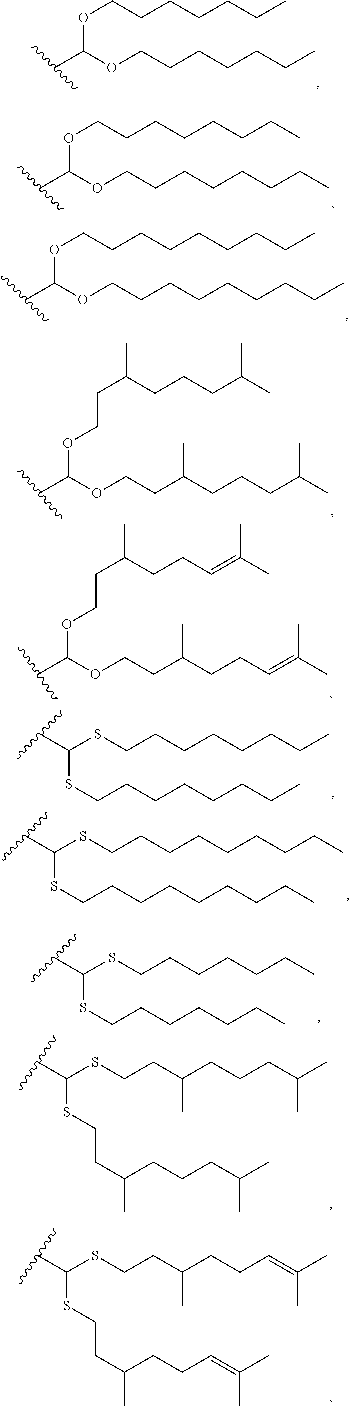

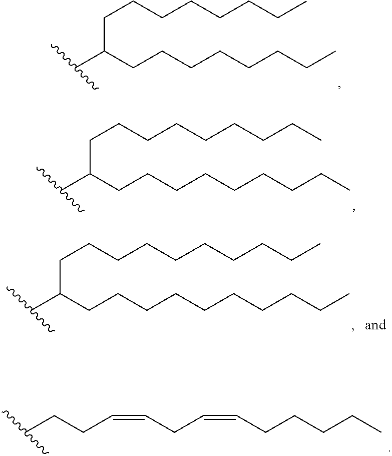 [SMILES: *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

In certain embodiments, R⁵ is selected from

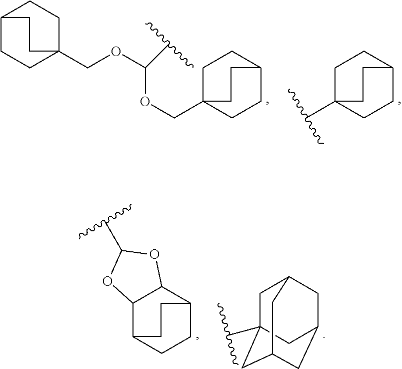

R⁶ and R⁷

As disclosed in Formula (CO), in certain embodiments, R⁶ and R⁷ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is optionally substituted C₁-C₁₄ aliphatic. In certain embodiments, R⁶ is optionally substituted C₁-C₁₄ alkyl. In certain embodiments, R⁶ is optionally substituted C₁-C₁₄ branched alkyl. In certain embodiments, R⁶ is optionally substituted C₁-C₁₄ straight chain alkyl. In certain embodiments, R⁶ is optionally substituted C₁-C₁₄ alkenyl. In certain embodiments, R⁶ is optionally substituted C₁-C₁₄ branched alkenyl. In certain embodiments, R⁶ is optionally substituted C₁-C₁₄ straight chain alkenyl. In certain embodiments, R⁶ is optionally substituted C₆-C₁₀ alkyl. In certain embodiments, R⁶ is optionally substituted —(CH₂)₅CH₃. In certain embodiments, R⁶ is optionally substituted —(CH₂)₆CH₃. In certain embodiments, R⁶ is optionally substituted —(CH₂)₇CH₃. In certain embodiments, R⁶ is optionally substituted —(CH₂) &CH₃. In certain embodiments, R⁶ is optionally substituted —(CH₂)₉CH₃.

In certain embodiments, one of the methylene linkages of R⁶ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

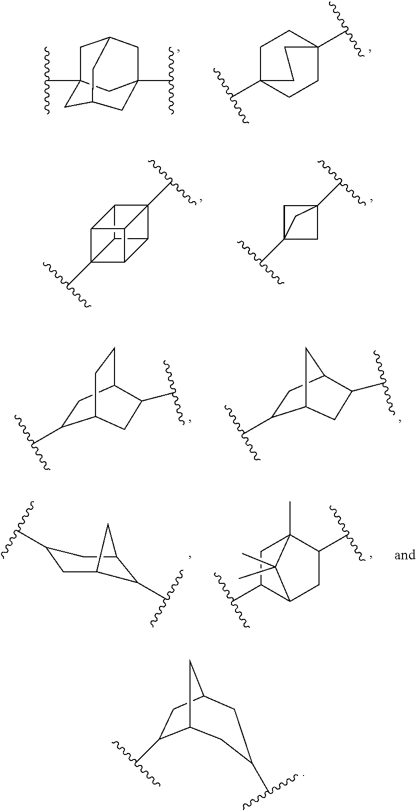

In certain embodiments, R⁷ is optionally substituted C₁-C₁₄ aliphatic. In certain embodiments, R⁷ is optionally substituted C₁-C₁₄ alkyl. In certain embodiments, R⁷ is optionally substituted C₁-C₁₄ branched alkyl. In certain embodiments, R⁷ is optionally substituted C₁-C₁₄ straight chain alkyl. In certain embodiments, R⁷ is optionally substituted C₁-C₁₄ alkenyl. In certain embodiments, R⁷ is optionally substituted C₁-C₁₄ branched alkenyl. In certain embodiments, R⁷ is optionally substituted C₁-C₁₄ straight chain alkenyl. In certain embodiments, R⁷ is optionally substituted C₆-C₁₀ alkyl. In certain embodiments, R⁷ is optionally substituted —(CH₂)₅CH₃. In certain embodiments, R⁷ is optionally substituted —(CH₂)₆CH₃. In certain embodiments, R⁷ is optionally substituted —(CH₂)₇CH₃. In certain embodiments, R⁷ is optionally substituted —(CH₂)₈CH₃. In certain embodiments, R⁶ is optionally substituted —(CH₂)₉CH₃.

In certain embodiments, one of the methylene linkages of R⁷ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

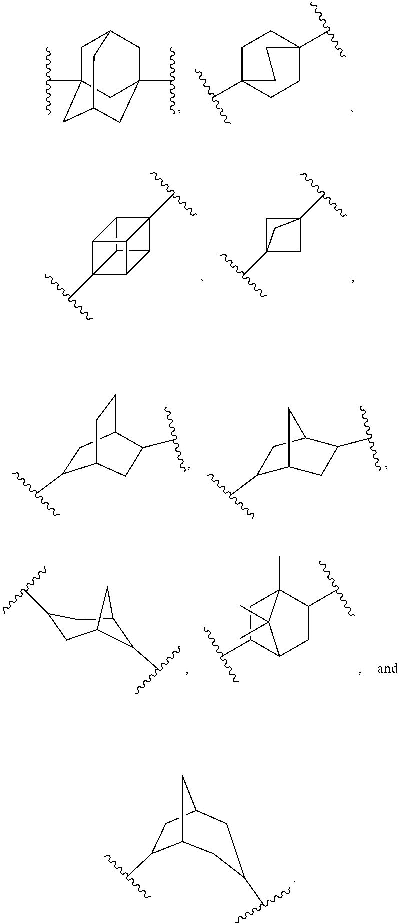

In certain embodiments, each R⁶ and R⁷ are selected from

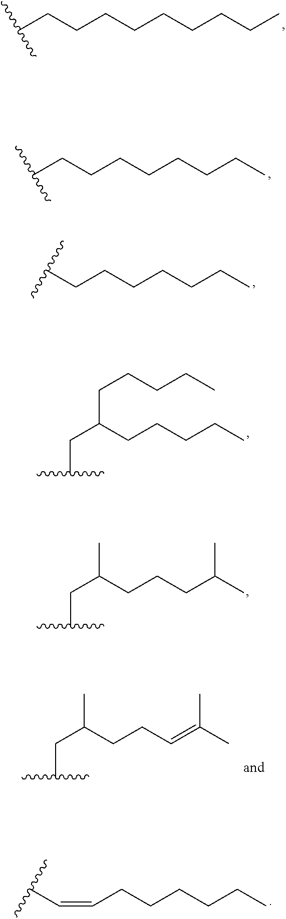

In certain embodiments, each R⁶ and R⁷ are each independently selected from an optionally substituted bridged bicyclic C₅-C₁₂ cycloalkylenyl. In certain embodiments, R⁶ is an optionally substituted bridged multicyclic C₅-C₁₂ cycloalkylenyl. In certain embodiments, R⁷ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

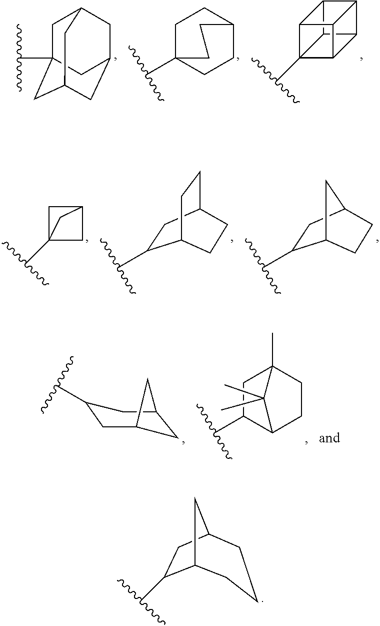

In certain embodiments, the substituted bridged bicyclic or multicyclic C₅-C₁₂ cycloalkylenyl is a structure selected from

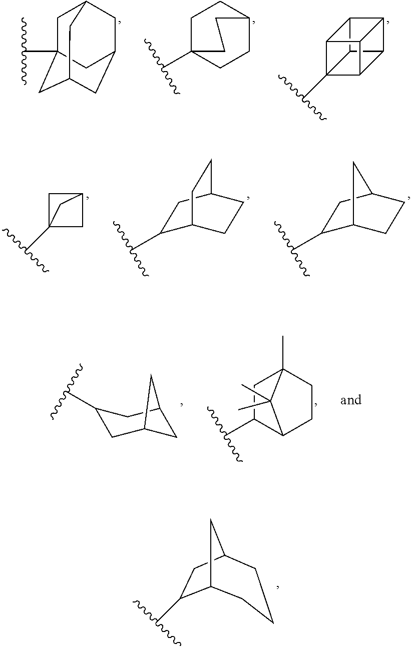

wherein one or more C—H bonds are substituted.

In certain embodiments, R⁶ and R⁷ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

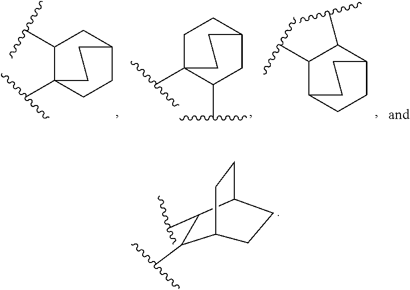

R⁸ and R⁹

As disclosed in Formula (CO), in certain embodiments, R⁸ and R⁹ are each independently optionally substituted C₁-C₁₄ aliphatic, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is optionally substituted C₁-C₁₄ aliphatic. In certain embodiments, R⁸ is optionally substituted C₁-C₁₄ alkyl. In certain embodiments, R⁸ is optionally substituted C₁-C₁₄ branched alkyl. In certain embodiments, R⁸ is optionally substituted C₁-C₁₄ straight chain alkyl. In certain embodiments, R⁸ is optionally substituted C₁-C₁₄ alkenyl. In certain embodiments, R⁸ is optionally substituted C₁-C₁₄ branched alkenyl. In certain embodiments, R⁸ is optionally substituted C₁-C₁₄ straight chain alkenyl. In certain embodiments, R⁸ is optionally substituted C₆-C₁₀ alkyl. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, one of the methylene linkages of R⁸ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

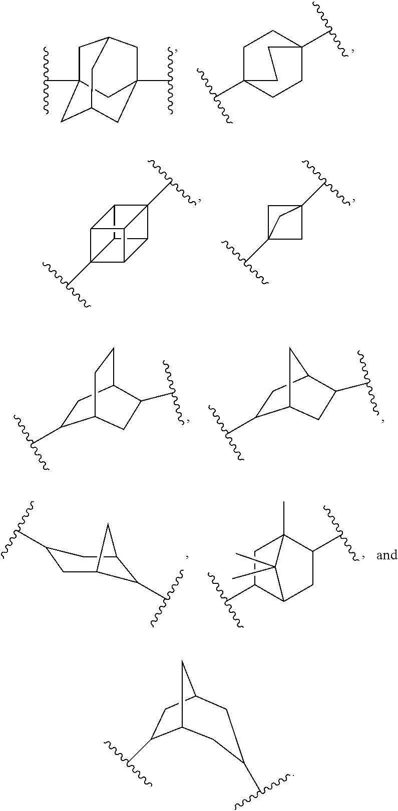

In certain embodiments, R⁹ is optionally substituted C₁-C₁₄ aliphatic. In certain embodiments, R⁹ is optionally substituted C₁-C₁₄ alkyl. In certain embodiments, R⁹ is optionally substituted C₁-C₁₄ branched alkyl. In certain embodiments, R⁹ is optionally substituted C₁-C₁₄ straight chain alkyl. In certain embodiments, R⁹ is optionally substituted C₁-C₁₄ alkenyl. In certain embodiments, R⁹ is optionally substituted C₁-C₁₄ branched alkenyl. In certain embodiments, R⁹ is optionally substituted C₁-C₁₄ straight chain alkenyl. In certain embodiments, R⁹ is optionally substituted C₆-C₁₀ alkyl. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)₇CH₃. In certain embodiments, R⁹ is optionally substituted —(CH₂)₈CH₃. In certain embodiments, R⁹ is optionally substituted —(CH₂)₉CH₃.

In certain embodiments, one of the methylene linkages of R⁹ is replaced with an optionally substituted C₃-C₈ cycloalkylenyl, an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

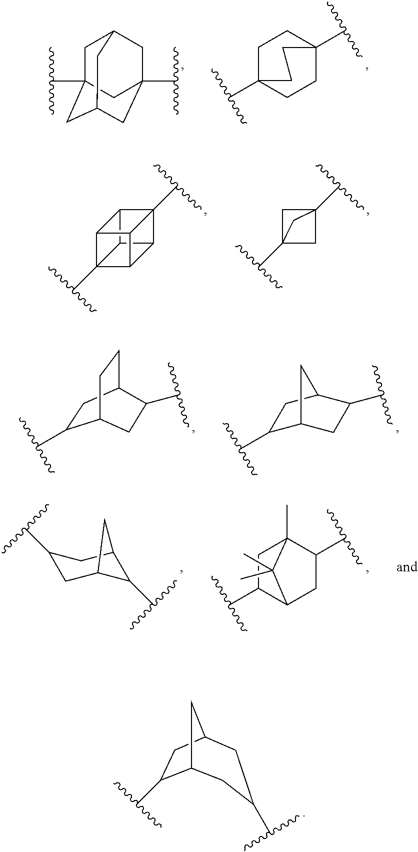

In certain embodiments, R⁸ and R⁹ are selected from

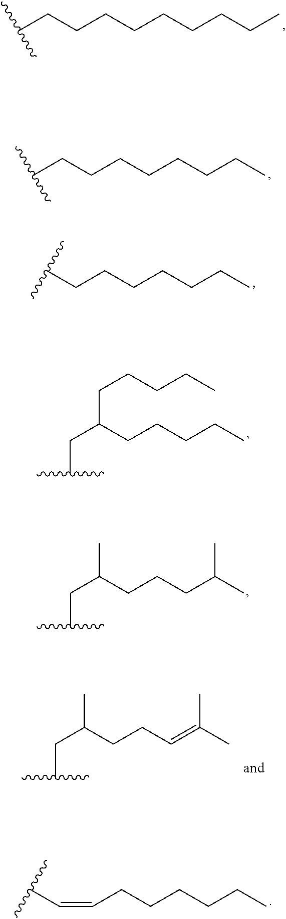

In some embodiments, R⁸ and R⁹ taken together form an optionally substituted bridged bicyclic or multicyclic C₄-C₁₄ cycloalkyl or optionally substituted bridged bicyclic or multicyclic 4-14 membered heterocyclyl.

In certain embodiments, each R⁸ and R⁹ are each independently selected from an optionally substituted bridged bicyclic C₅-C₁₂ cycloalkylenyl. In certain embodiments, R⁸ is an optionally substituted bridged multicyclic C₅-C₁₂ cycloalkylenyl. In certain embodiments, R⁹ is an optionally substituted bridged bicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

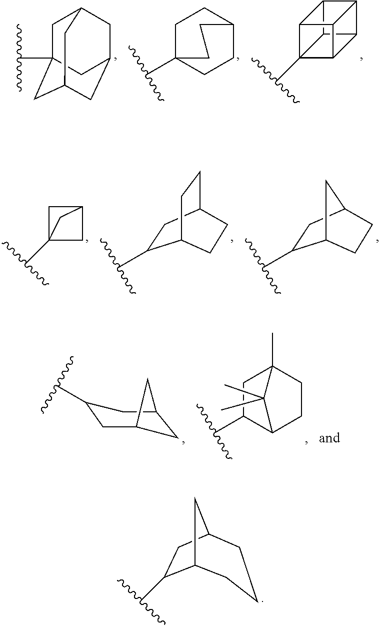

In certain embodiments, the substituted bridged bicyclic or multicyclic C₅-C₁₂ cycloalkylenyl is a structure selected from

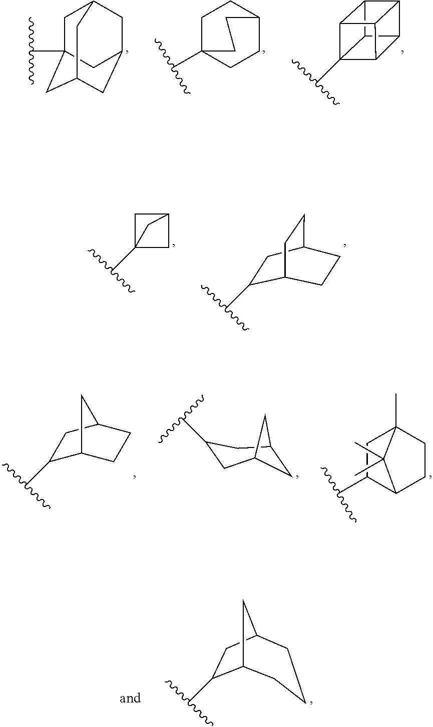

wherein one or more C—H bonds are substituted.

In certain embodiments, R⁸ and R⁹ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

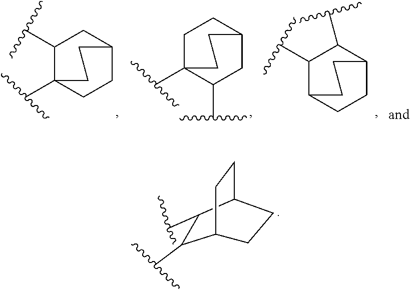

In some embodiments, Lipids of the Present Disclosure are selected from any lipid in Table (IX) below or a pharmaceutically acceptable salt thereof:

TABLE (IX)
Non-Limiting Examples of Ionizable Lipids of the Present Disclosure

| Cmpd | Structure |
|---|---|
| CO-1 | 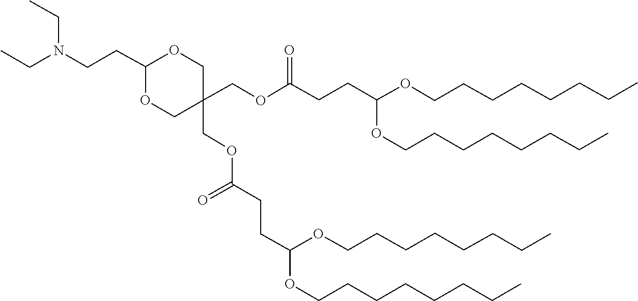 [SMILES: CCCCCCCCOC(CCC(=O)OCC1(COC(=O)CCC(OCCCCCCCC)OCCCCCCCC)COC(CCN(CC)CC)OC1)OCCCCCCCC] |
| CO-2 | 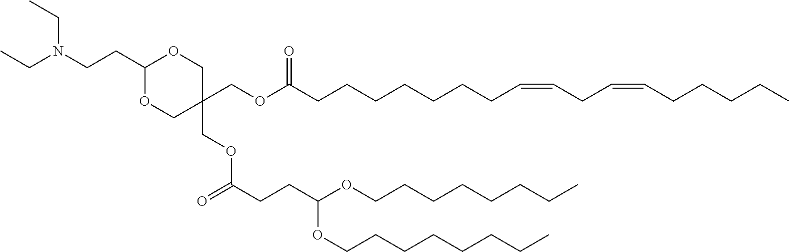 [SMILES: CCCCC/C=C\C/C=C\CCCCCCCC(=O)OCC1(COC(=O)CCC(OCCCCCCCC)OCCCCCCCC)COC(CCN(CC)CC)OC1] |
| CO-3 | 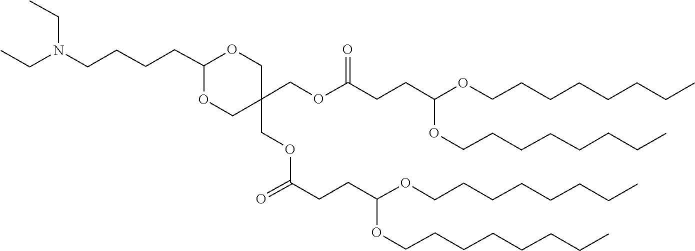 [SMILES: CCCCCCCCOC(CCC(=O)OCC1(COC(=O)CCC(OCCCCCCCC)OCCCCCCCC)COC(CCCCN(CC)CC)OC1)OCCCCCCCC] |
| CO-4 | 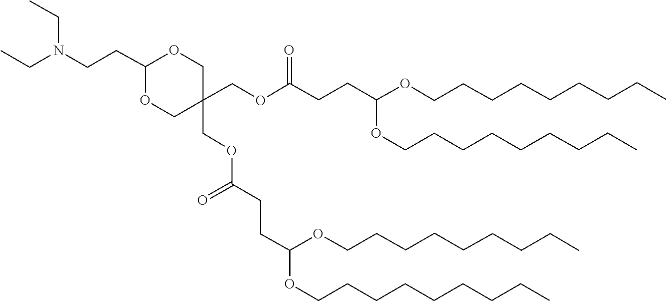 [SMILES: CCCCCCCCCOC(CCC(=O)OCC1(COC(=O)CCC(OCCCCCCCCC)OCCCCCCCCC)COC(CCN(CC)CC)OC1)OCCCCCCCCC] |
| CO-5 | 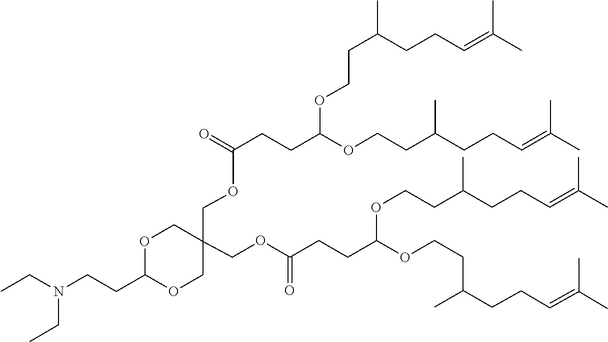 [SMILES: CCN(CC)CCC1OCC(COC(=O)CCC(OCCC(C)CCC=C(C)C)OCCC(C)CCC=C(C)C)(COC(=O)CCC(OCCC(C)CCC=C(C)C)OCCC(C)CCC=C(C)C)CO1] |
| CO-6 | 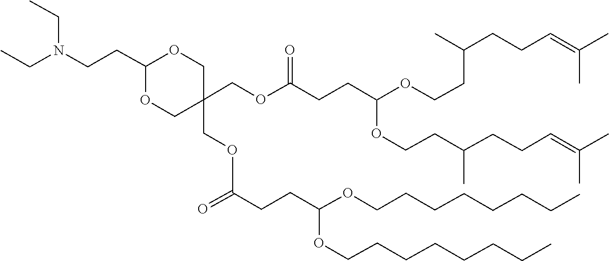 [SMILES: CCCCCCCCOC(CCC(=O)OCC1(COC(=O)CCC(OCCC(C)CCC=C(C)C)OCCC(C)CCC=C(C)C)COC(CCN(CC)CC)OC1)OCCCCCCCC] |
| CO-7 | 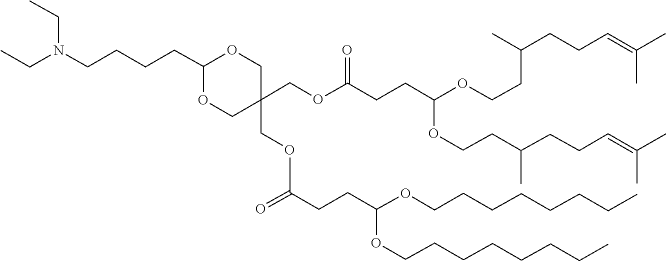 [SMILES: CCCCCCCCOC(CCC(=O)OCC1(COC(=O)CCC(OCCC(C)CCC=C(C)C)OCCC(C)CCC=C(C)C)COC(CCCCN(CC)CC)OC1)OCCCCCCCC] |
| CO-8 | 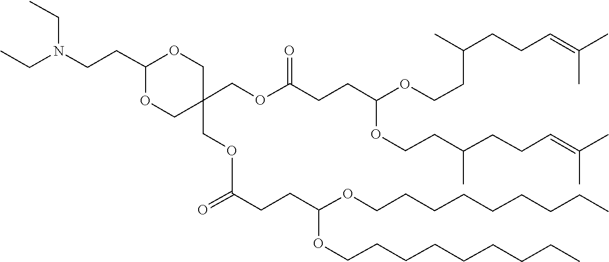 [SMILES: CCCCCCCCCOC(CCC(=O)OCC1(COC(=O)CCC(OCCC(C)CCC=C(C)C)OCCC(C)CCC=C(C)C)COC(CCN(CC)CC)OC1)OCCCCCCCCC] |
| CO-9 | 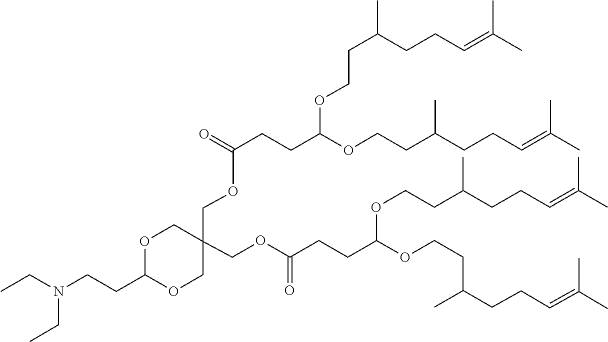 [SMILES: CCN(CC)CCC1OCC(COC(=O)CCC(OCCC(C)CCC=C(C)C)OCCC(C)CCC=C(C)C)(COC(=O)CCC(OCCC(C)CCC=C(C)C)OCCC(C)CCC=C(C)C)CO1] |
| CO-10 | 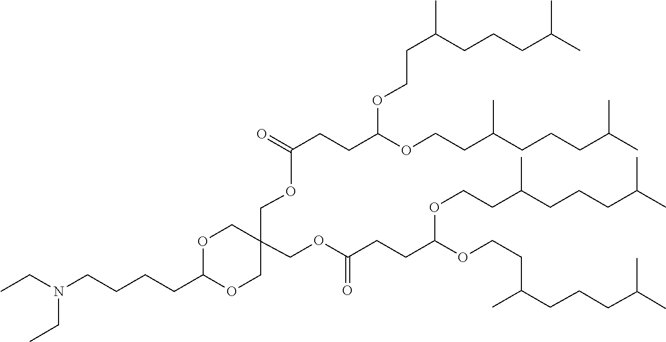 [SMILES: CCN(CC)CCCCC1OCC(COC(=O)CCC(OCCC(C)CCCC(C)C)OCCC(C)CCCC(C)C)(COC(=O)CCC(OCCC(C)CCCC(C)C)OCCC(C)CCCC(C)C)CO1] |
| CO-11 | 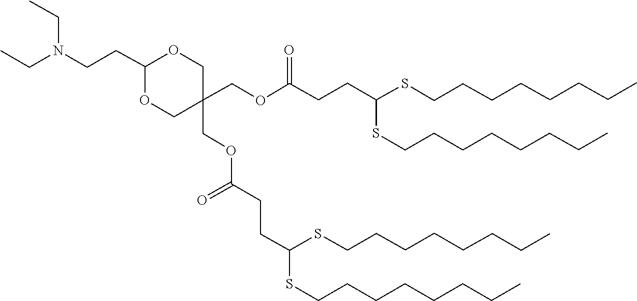 [SMILES: CCCCCCCCSC(CCC(=O)OCC1(COC(=O)CCC(SCCCCCCCC)SCCCCCCCC)COC(CCN(CC)CC)OC1)SCCCCCCCC] |

### Series “CC”

In some embodiments, an LNP of the present disclosure comprises an ionizable lipid disclosed in PCT Application PCT/US2024/019990, which is incorporated by reference herein, in its entirety.

The present disclosure provides compound of Formula (CC)

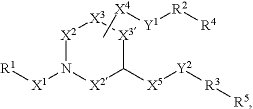

or a pharmaceutically acceptable salt thereof, wherein:
R¹ is selected from the group consisting of —OH, —OAc, —NR₂,

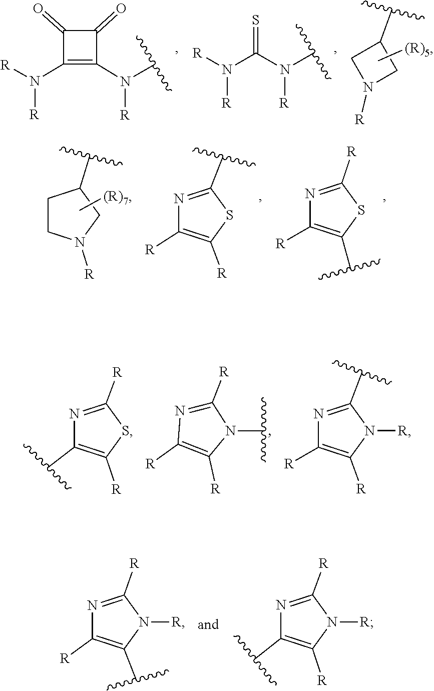

each R is independently —H or C₁-C₆ aliphatic;
X¹ is optionally substituted C₂-C₆ aliphatic, wherein one or more methylene linkages are each optionally and independently replaced with —O—, —NH—, —S—, —SS—, —C(O)—, —OC(O)O—, —OC(O)—, —NHC(O)— or —C(O)O—;
X² is selected from the group consisting of a bond, —CH₂— and —CH₂CH₂—;
X^{2′} is selected from the group consisting of a bond, —CH₂— and —CH₂CH₂—;
X³ is selected from the group consisting of a bond, —CH₂— and —CH₂CH₂—;
X^{3′} is selected from the group consisting of a bond, —CH₂— and —CH₂CH₂—;
X⁴ and X⁵ are independently optionally substituted C₁-C₁₀ aliphatic;
Y¹ and Y² are independently selected from the group consisting of

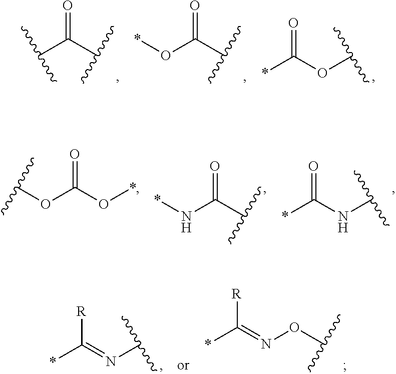

wherein the bond marked with an “*” is attached to X⁴ or X⁵;
R² is optionally substituted C₁-C₆ aliphatic;
R³ is optionally substituted C₁-C₆ aliphatic;
R⁴ is —CH(OR⁶)(OR⁷); —CH(SR⁶)(SR⁷); —CH(SR⁸)(SR⁹); —CH(R⁶)(R⁷); —R¹⁰; or optionally substituted C₁-C₁₄ aliphatic-R¹⁰ wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—;
R⁵ is —CH(OR⁸)(OR⁹); —CH(SR⁸)(SR⁹); —CH(R⁸)(R⁹); optionally substituted C₁-C₁₄ aliphatic, wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; —R¹¹; or optionally substituted C₁-C₁₄ aliphatic-R¹¹, wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—;
R⁶ and R⁷ are each independently —R¹⁰; optionally substituted-C₁-C₁₄ aliphatic-R¹⁰; wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—;
R⁸ and R⁹ are each independently —R¹¹; optionally substituted-C₁-C₁₄ aliphatic wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; or optionally substituted-C₁-C₁₄ aliphatic-R¹¹ wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; and
each R¹⁰ and R¹¹ is independently an optionally substituted bridged bicyclic or multicyclic C₅-C₁₂ cycloalkylenyl, or two R¹⁰ or two R¹¹ taken together form an optionally substituted bridged bicyclic or multicyclic C₅-C₁₂ cycloalkylenyl; or
each R¹⁰ and R¹¹ is independently an optionally substituted cyclic, bicyclic, bridged bicyclic, multicyclic or bridged multicyclic C₄-C₁₄ cycloalkyl or optionally substituted cyclic, bicyclic, bridged bicyclic, multicyclic or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl.

### Additional Formulae

In certain embodiments, the compound of Formula (CC) is a compound of any one of the Formulae below:

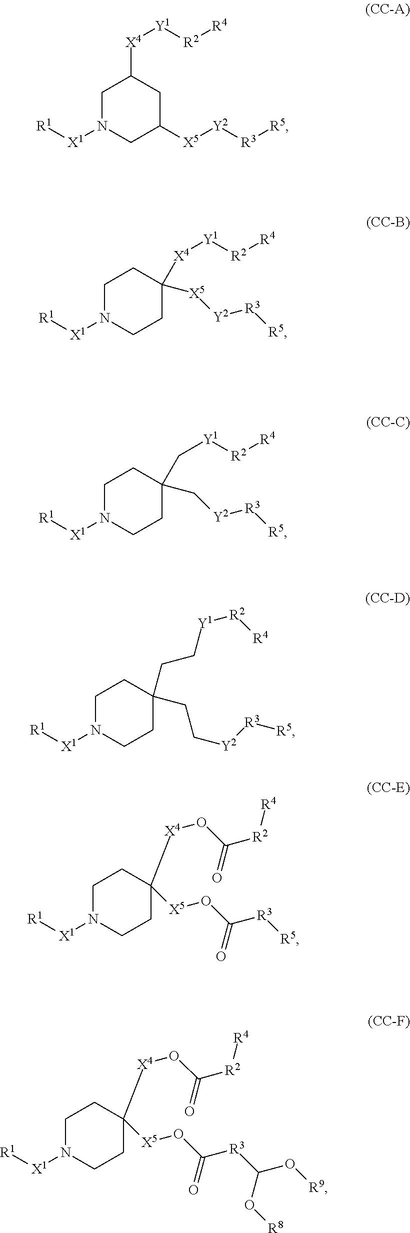

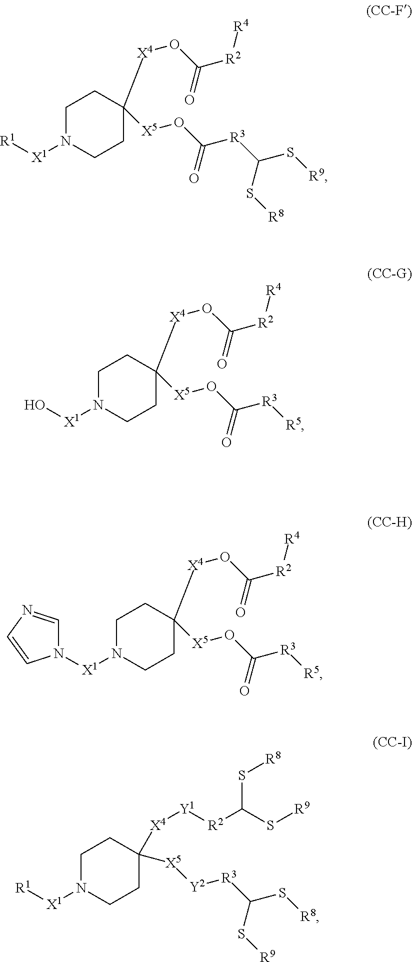

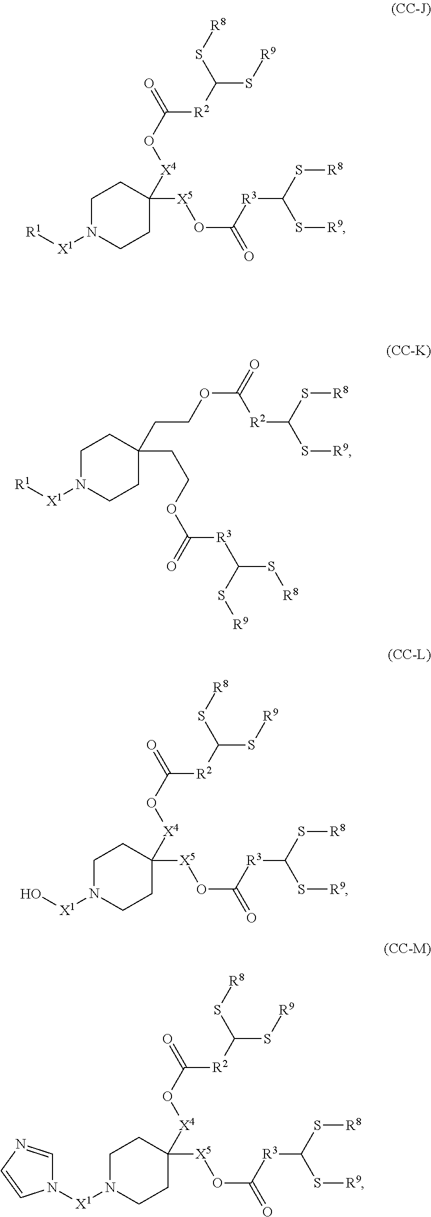

or a pharmaceutically acceptable salt thereof, wherein R¹, R, X¹, X⁴, X⁵, Y¹, Y², R², R³, R⁴, R⁵, R⁶, R⁷, R⁸, R⁹, R¹⁰, R¹¹ are as described in Formula (CC) or (CC′) or as otherwise described in any embodiments below.

### R¹

As disclosed in Formula (CC), in certain embodiments, R¹ is selected from the group consisting of —OH, —OAc, —NR₂,

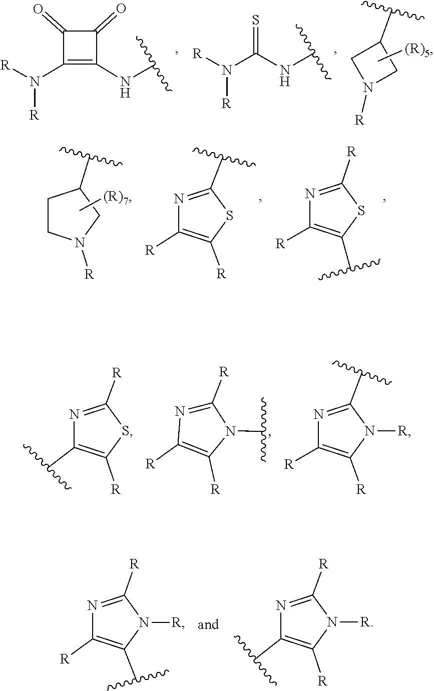

In certain embodiments, R¹ is —OH. In certain embodiments, R¹ is —OAc. In certain embodiments, R¹ is —NR². In certain embodiments, R¹ is

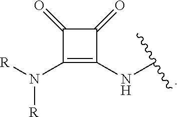

In certain embodiments, R¹ is

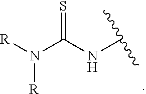

In certain embodiments, R¹ is

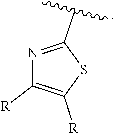

In certain embodiments, R¹ is

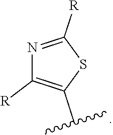

In certain embodiments, R¹ is

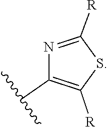

In certain embodiments, R¹ is

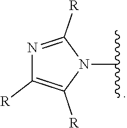

In certain embodiments, R¹ is

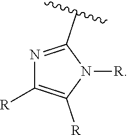

In certain embodiments, R¹ is

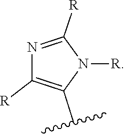

In certain embodiments, R¹ is

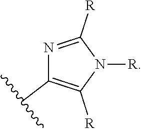

In certain embodiments, R¹ is

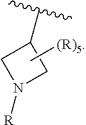

In certain embodiments, R¹ is

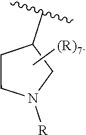

In certain embodiments, R¹ is —N(Et)₂. In certain embodiments, R¹ is —N(Me)₂. In certain embodiments, R¹ is —NH₂. In certain embodiments, R¹ is —N(nPr)₂. In certain embodiments, R¹ is —N(iPr)₂. In certain embodiments, R¹ is —N(Me)(Et). In certain embodiments, R¹ is OH. In certain embodiments, R¹ is

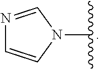 [SMILES: *n1ccnc1]

### X¹

As disclosed in Formula (CC), in certain embodiments, X¹ is optionally substituted C₂-C₆ aliphatic, wherein one or more methylene linkages are each optionally and independently replaced with —O—, —NH—, —S—, —SS—, —C(O)—, —OC(O)O—, —NHC(O)— or —C(O)O—. In certain embodiments, X¹ is optionally substituted C₂-C₆ aliphatic. In certain embodiments, X¹ is optionally substituted C₂-C₆ alkylene. In certain embodiments, X¹ is optionally substituted C₂ alkylene. In certain embodiments, X¹ is optionally substituted C₃ alkylene. In certain embodiments, X¹ is optionally substituted C₄ alkylene. In certain embodiments, X¹ is optionally substituted C₅ alkylene. In certain embodiments, X¹ is optionally substituted C₆ alkylene. In certain embodiments, X¹ is —(CH₂)₂—. In certain embodiments, X¹ is —(CH₂)₃—. In certain embodiments, X¹ is —(CH₂)₄—. In certain embodiments, X¹ is —(CH₂)₅—. In certain embodiments, X¹ is —(CH₂)₆—.

### X²

As disclosed in Formula (CC), in certain embodiments, X² is selected from the group consisting of a bond, —CH₂— and —CH₂CH₂—. In certain embodiments, X² is a bond. In certain embodiments, X² is —CH₂—. In certain embodiments, X² is —CH₂CH₂—.

### X^{2′}

As disclosed in Formula (CC), in certain embodiments, X² is selected from the group consisting of a bond, —CH₂— and —CH₂CH₂—. In certain embodiments, X^{2′} is a bond. In certain embodiments, X^{2′} is —CH₂—. In certain embodiments, X² is —CH₂CH₂—.

### X³

As disclosed in Formula (CC), in certain embodiments, X³ is selected from the group consisting of a bond, —CH₂— and —CH₂CH₂—. In certain embodiments, X³ is a bond. In certain embodiments, X³ is —CH₂—. In certain embodiments, X³ is —CH₂CH₂—.

### X^{3′}

As disclosed in Formula (CC), in certain embodiments, X³ is selected from the group consisting of a bond, —CH₂— and —CH₂CH₂—. In certain embodiments, X^{3′} is a bond. In certain embodiments, X^{3′} is —CH₂—. In certain embodiments, X^{3′} is —CH₂CH₂—.

In certain embodiments, each of X², X^{2′}, X³ and X^{3′} are each —CH₂—. In certain embodiments, both X² and X³ are each —CH₂—; X^{3′} is a bond, and X^{2′} is —CH₂CH₂—.

X⁴ and X⁵

As disclosed in Formula (CC), in certain embodiments, X⁴ and X⁵ are each independently optionally substituted C₁-C₁₀ aliphatic. In certain embodiments, X⁴ and X⁵ are the same. In certain embodiments, X⁴ and X⁵ are different.

In certain embodiments, X⁴ is an optionally substituted C₁-C₁₀ alkylene. In certain embodiments, X⁴ is an optionally substituted C₁-C₁₀ alkenylene. In certain embodiments, X⁴ is an optionally substituted C₁-C₆ alkylene. In certain embodiments, X⁴ is an optionally substituted C₁-C₆ alkenylene. In certain embodiments, X⁴ is —(CH₂)—. In certain embodiments, X⁴ is —(CH₂)₂—. In certain embodiments, X⁴ is —(CH₂)₃—. In certain embodiments, X⁴ is —(CH₂)₄—. In certain embodiments, X⁴ is —(CH₂)₅—. In certain embodiments, X⁴ is —(CH₂)₆—.

In certain embodiments, X⁵ is an optionally substituted C₁-C₁₀ alkylene. In certain embodiments, X⁵ is an optionally substituted C₁-C₁₀ alkenylene. In certain embodiments, X⁵ is an optionally substituted C₁-C₆ alkylene. In certain embodiments, X⁵ is an optionally substituted C₁-C₆ alkenylene. In certain embodiments, X⁵ is —(CH₂)—. In certain embodiments, X⁵ is —(CH₂)₂—. In certain embodiments, X⁵ is —(CH₂)₃—. In certain embodiments, X⁵ is —(CH₂)₄—. In certain embodiments, X⁵ is —(CH₂)₅—. In certain embodiments, X⁵ is —(CH₂)₆—.

In certain embodiments, X⁴ and X⁵ are both —(CH₂)—. In certain embodiments, X⁴ and X⁵ are both —(CH₂)₂—.

Y¹ and Y²

As disclosed in Formula (CC), in certain embodiments, Y¹ and Y² are each independently

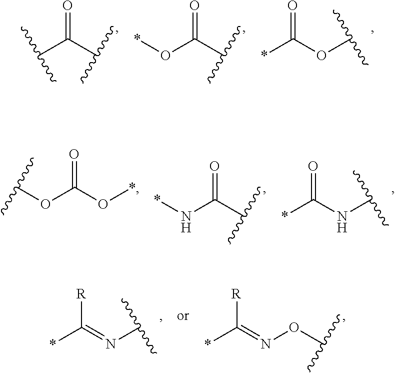

wherein the bond marked with an “*” is attached to X⁴ or X⁵. In certain embodiments, Y¹ and Y² are the same. In certain embodiments, Y¹ and Y² are different.

In certain embodiments, Y¹ is

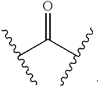 [SMILES: *C(*)=O]

In certain embodiments, Y¹ is

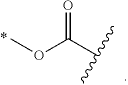 [SMILES: *C(=O)OC]

In certain embodiments, Y¹ is

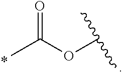 [SMILES: *OC(C)=O]

In certain embodiments, Y¹ is

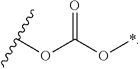

In certain embodiments, Y¹ is

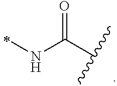

In certain embodiments, Y¹ is

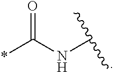 [SMILES: *NC(*)=O]

In certain embodiments, Y¹ is

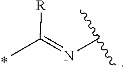

In certain embodiments, Y¹ is

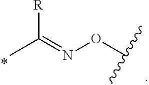

In certain embodiments, Y² is

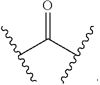 [SMILES: *C(*)=O]

In certain embodiments, Y² is

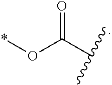 [SMILES: *OC(*)=O]

In certain embodiments, Y² is

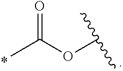 [SMILES: *OC(C)=O]

In certain embodiments, Y² is

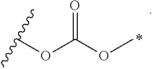

In certain embodiments, Y² is

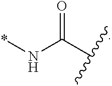 [SMILES: *NC(*)=O ; "."]

In certain embodiments, Y² is

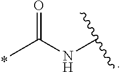

In certain embodiments, Y² is

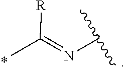

In certain embodiments, Y² is

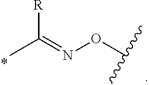

In certain embodiments, Y¹ and Y² are both

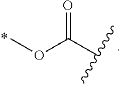 [SMILES: *C(=O)OC]

In certain embodiments, Y¹ and Y² are both

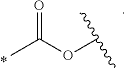 [SMILES: *OC(C)=O]

### R²

As disclosed in Formula (CC), in certain embodiments, R² is optionally substituted C₁-C₆ aliphatic. In certain embodiments, R² is optionally substituted C₁-C₆ alkylene. In certain embodiments, R² is optionally substituted methylene. In certain embodiments, R² is optionally substituted C₂ alkylene. In certain embodiments, R² is optionally substituted C₃ alkylene. In certain embodiments, R² is optionally substituted C₄ alkylene. In certain embodiments, R² is optionally substituted C₅ alkylene. In certain embodiments, R² is optionally substituted C₆ alkylene. In certain embodiments, R² is —(CH₂)—. In certain embodiments, R² is —(CH₂)₂—. In certain embodiments, R² is —(CH₂)₃—. In certain embodiments, R² is —(CH₂)₄—. In certain embodiments, R² is —(CH₂)₅—. In certain embodiments, R² is —(CH₂)₆—.

### R³

As disclosed in Formula (CC), in certain embodiments, R³ is optionally substituted C₁-C₆ aliphatic. In certain embodiments, R³ is optionally substituted C₁-C₆ alkylene. In certain embodiments, R³ is optionally substituted methylene. In certain embodiments, R³ is optionally substituted C₂ alkylene. In certain embodiments, R³ is optionally substituted C₃ alkylene. In certain embodiments, R³ is optionally substituted C₄ alkylene. In certain embodiments, R³ is optionally substituted C₅ alkylene. In certain embodiments, R³ is optionally substituted C₆ alkylene. In certain embodiments, R³ is —(CH₂)—. In certain embodiments, R³ is —(CH₂)₂—. In certain embodiments, R³ is —(CH₂)₃—. In certain embodiments, R³ is —(CH₂)₄—. In certain embodiments, R³ is —(CH₂)₅—. In certain embodiments, R³ is —(CH₂)₆—.

In certain embodiments, R² and R³ are the same. In certain embodiments, R² and R³ are different. In certain embodiments, R² and R³ are both —(CH₂)₂—.

### R⁴

As disclosed in Formula (CC), in certain embodiments, R⁴ is —CH(OR⁶)(OR⁷); —CH(SR⁶)(SR⁷); —CH(SR⁸)(SR⁹); —CH(R⁶)(R⁷); —R¹⁰; or optionally substituted C₁-C₁₄ aliphatic-R¹⁰ wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic-R¹⁰, wherein one or more methylene linkages are each optionally and independently replaced with an optionally substituted C₃-C₈ cycloalkylenyl, an optionally substituted bridged bicyclic or multicyclic C₅-C₁₂ cycloalkylenyl, phenyl, —O—, —NH—, —S—, —SS—, —C(O)—, —OC(O)O—, —OC(O)—, —NHC(O)— or —C(O)O—. In certain embodiments, R⁴ is optionally substituted C₁-C₁₄ aliphatic-R¹⁰. In certain embodiments, R⁴ is —CH(OR⁶)(OR⁷). In certain embodiments, R⁴ is —CH(R⁶)(R⁷). In certain embodiments, R⁴ is —CH(SR⁶)(SR⁷). In certain embodiments, R⁴ is —CH(SR⁸)(SR⁹). In certain embodiments, R⁴ is R¹⁰.

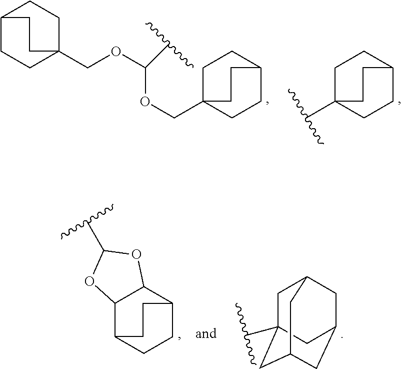 [SMILES: *C(OCC12CCC(CC1)CC2)OCC12CCC(CC1)CC2 ; *C12CCC(CC1)CC2 ; *C1OC2C3CCC(CC3)C2O1 ; *C12CC3CC(CC(C3)C1)C2]

In certain embodiments, R⁴ is selected from

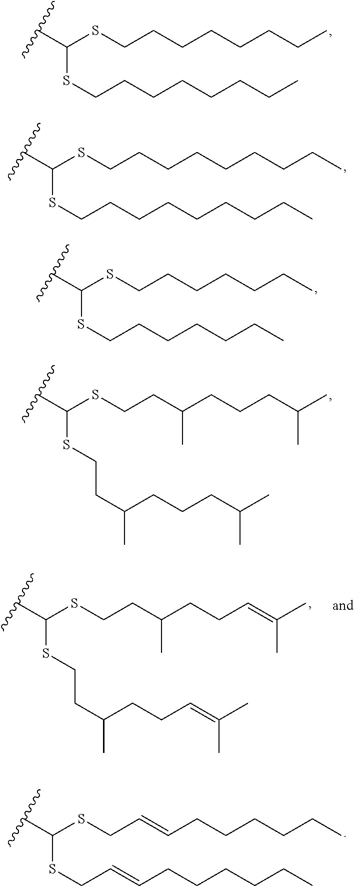 [SMILES: *C(SCCCCCCCC)SCCCCCCCC ; *C(SCCCCCCCCC)SCCCCCCCCC ; *C(SCCCCCCC)SCCCCCCC ; *C(SCCC(C)CCCC(C)C)SCCC(C)CCCC(C)C ; *C(SCCC(C)CCC=C(C)C)SCCC(C)CCC=C(C)C ; *C(SC/C=C/CCCCCC)SC/C=C/CCCCCC]

In certain embodiments, R⁴ is selected from

### R⁵

As disclosed in Formula (CC), in certain embodiments, R⁵ is —CH(OR⁸)(OR⁹); —CH(SR⁸)(SR⁹); —CH(R⁸)(R⁹); optionally substituted C₁-C₁₄ aliphatic, wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; —R¹¹; or optionally substituted C₁-C₁₄ aliphatic-R¹¹, wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—. In certain embodiments, R⁵ is optionally substituted C₁-C₁₄ aliphatic. In certain embodiments, R⁵ is —CH(OR⁸)(OR⁹). In certain embodiments, R⁵ is —CH(R⁸)(R⁹). In certain embodiments, R⁵ is —CH(SR⁸)(SR⁹). In certain embodiments, R⁵ is R¹¹.

In certain embodiments, R⁴ and R⁵ are the same. In certain embodiments, R⁴ and R⁵ are different.

In certain embodiments, R⁵ is selected from

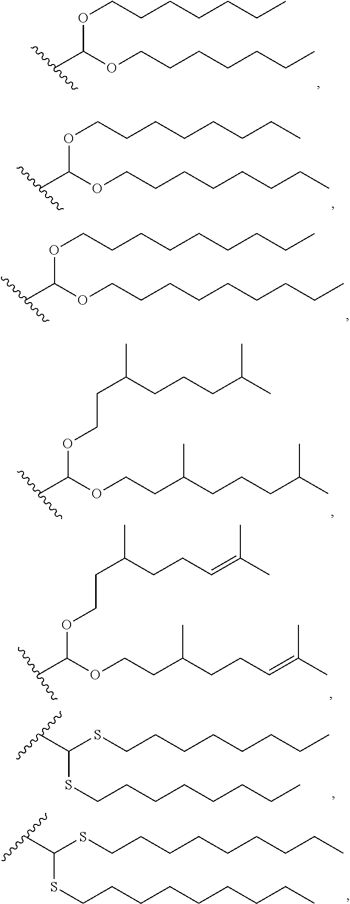

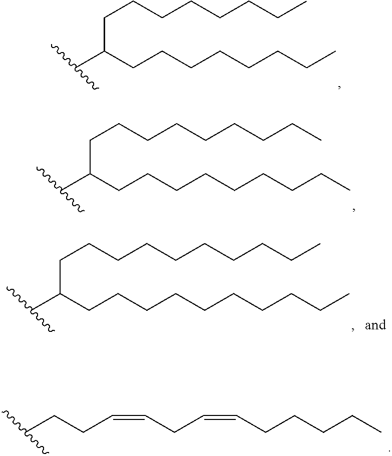 [SMILES: *C(CCCCCCCC)CCCCCCCC ; *C(CCCCCCCCC)CCCCCCCCC ; *C(CCCCCCCCCC)CCCCCCCCCC ; *CC/C=C\C/C=C\CCCCC]

In certain embodiments, R⁵ is selected from

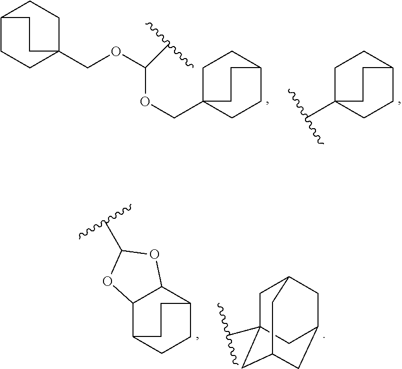

R⁶ and R⁷

As disclosed in Formula (CC), in certain embodiments, R⁶ and R⁷ are each independently —R¹⁰; optionally substituted-C₁-C₁ aliphatic-R¹⁰; wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—.

In certain embodiments, R⁶ and R⁷ are the same. In certain embodiments, R⁶ and R⁷ are different.

In certain embodiments, R⁶ is R¹⁰. In certain embodiments, R⁶ is optionally substituted C₁-C₁₄ aliphatic-R¹⁰. In certain embodiments, R⁶ is optionally substituted C₁-C₁₄ alkyl-R¹⁰. In certain embodiments, R⁶ is optionally substituted C₁-C₁₄ branched alkyl-R¹⁰. In certain embodiments, R⁶ is optionally substituted C₁-C₁₄ straight chain alkyl-R¹⁰. In certain embodiments, R⁶ is optionally substituted C₁-C₁₄ alkenyl-R¹⁰. In certain embodiments, R⁶ is optionally substituted C₁-C₁₄ branched alkenyl-R¹⁰. In certain embodiments, R⁶ is optionally substituted C₁-C₁₄ straight chain alkenyl-R¹⁰. In certain embodiments, R⁶ is optionally substituted C₁-C₅ alkyl-R¹⁰. In certain embodiments, R⁶ is optionally substituted —(CH₂)—R¹⁰. In certain embodiments, R⁶ is optionally substituted —(CH₂)₂—R¹⁰. In certain embodiments, R⁶ is optionally substituted —(CH₂)₃—R¹⁰. In certain embodiments, R⁶ is optionally substituted —(CH₂)₄—R¹⁰. In certain embodiments, R⁶ is optionally substituted —(CH₂)₅—R¹⁰.

In certain embodiments, R⁷ is R¹⁰. In certain embodiments, R⁷ is optionally substituted C₁-C₁₄ aliphatic-R¹⁰. In certain embodiments, R⁷ is optionally substituted C₁-C₁₄ alkyl-R¹⁰. In certain embodiments, R⁷ is optionally substituted C₁-C₁₄ branched alkyl-R¹⁰. In certain embodiments, R⁷ is optionally substituted C₁-C₁₄ straight chain alkyl-R¹⁰. In certain embodiments, R⁷ is optionally substituted C₁-C₁₄ alkenyl-R¹⁰. In certain embodiments, R⁷ is optionally substituted C₁-C₁₄ branched alkenyl-R¹⁰. In certain embodiments, R⁷ is optionally substituted C₁-C₁₄ straight chain alkenyl-R¹⁰. In certain embodiments, R⁷ is optionally substituted C₁-C₅ alkyl-R¹⁰. In certain embodiments, R⁷ is optionally substituted —(CH₂)—R¹⁰. In certain embodiments, R⁷ is optionally substituted —(CH₂)₂—R¹⁰. In certain embodiments, R⁷ is optionally substituted —(CH₂)₃—R¹⁰. In certain embodiments, R⁷ is optionally substituted —(CH₂)₄—R¹⁰. In certain embodiments, R⁷ is optionally substituted —(CH₂)₅—R¹⁰.

In certain embodiments, R⁶ and R⁷ are selected from

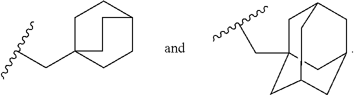 [SMILES: *CC12CCC(CC1)CC2 ; *CC12CC3CC(CC(C3)C1)C2]

R⁸ and R⁹

As disclosed in Formula (CC), in certain embodiments, R⁸ and R⁹ are each independently R¹¹; optionally substituted-C₁-C₁₄ aliphatic wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—; or optionally substituted-C₁-C₁₄ aliphatic-R¹¹ wherein one or more methylene linkages are each optionally and independently replaced with an optionally substituted C₃-C₈ cycloalkylenyl, phenyl, —O—, —NH—, —S—, —SS—, —C(O)—, —OC(O)O—, —OC(O)—, —NHC(O)— or —C(O)O—.

In certain embodiments, R⁸ and R⁹ are the same. In certain embodiments, R⁸ and R⁹ are different.

In certain embodiments, R⁸ is R¹¹. In certain embodiments, R⁸ is optionally substituted C₁-C₁₄ aliphatic. In certain embodiments, R⁸ is optionally substituted C₁-C₁₄ alkyl. In certain embodiments, R⁸ is optionally substituted C₁-C₁₄ branched alkyl. In certain embodiments, R⁸ is optionally substituted C₁-C₁₄ straight chain alkyl. In certain embodiments, R⁸ is optionally substituted C₁-C₁₄ alkenyl. In certain embodiments, R⁸ is optionally substituted C₁-C₁₄ branched alkenyl. In certain embodiments, R⁸ is optionally substituted C₁-C₁₄ straight chain alkenyl. In certain embodiments, R⁸ is optionally substituted C₆-C₁₀ alkyl. In certain embodiments, R⁸ is optionally substituted —(CH₂)₅CH₃. In certain embodiments, R⁸ is optionally substituted —(CH₂)₆CH₃. In certain embodiments, R⁸ is optionally substituted —(CH₂)₇CH₃. In certain embodiments, R⁸ is optionally substituted —(CH₂)₈CH₃. In certain embodiments, R⁸ is optionally substituted —(CH₂)₉CH₃.

In certain embodiments, R⁸ is optionally substituted C₁-C₁₄ aliphatic-R¹¹. In certain embodiments, R⁸ is optionally substituted C₁-C₁₄ alkylene-R¹¹. In certain embodiments, R⁸ is optionally substituted C₁-C₁₄ branched alkylene-R¹¹. In certain embodiments, R⁸ is optionally substituted C₁-C₁₄ straight chain alkylene-R¹¹. In certain embodiments, R⁸ is optionally substituted C₁-C₁₄ alkenylene-R¹¹. In certain embodiments, R⁸ is optionally substituted C₁-C₁₄ branched alkenylene-R¹¹. In certain embodiments, R⁸ is optionally substituted C₁-C₁₄ straight chain alkenylene-R¹¹. In certain embodiments, R⁸ is optionally substituted C₁-C₅ alkylene-R¹¹. In certain embodiments, R⁸ is optionally substituted —(CH₂)—R¹¹. In certain embodiments, R⁸ is optionally substituted —(CH₂)₂—R¹¹. In certain embodiments, R⁸ is optionally substituted —(CH₂)₃—R¹¹. In certain embodiments, R⁸ is optionally substituted —(CH₂)₄—R¹¹. In certain embodiments, R⁸ is optionally substituted —(CH₂)₅—R¹¹.

In certain embodiments, R⁹ is R¹¹. In certain embodiments, R⁹ is optionally substituted C₁-C₁₄ aliphatic. In certain embodiments, R⁹ is optionally substituted C₁-C₁₄ alkyl. In certain embodiments, R⁹ is optionally substituted C₁-C₁₄ branched alkyl. In certain embodiments, R⁹ is optionally substituted C₁-C₁₄ straight chain alkyl. In certain embodiments, R⁹ is optionally substituted C₁-C₁₄ alkenyl. In certain embodiments, R⁹ is optionally substituted C₁-C₁₄ branched alkenyl. In certain embodiments, R⁹ is optionally substituted C₁-C₁₄ straight chain alkenyl. In certain embodiments, R⁹ is optionally substituted C₆-C₁₀ alkyl. In certain embodiments, R⁹ is optionally substituted —(CH₂)₅CH₃. In certain embodiments, R⁹ is optionally substituted —(CH₂)₆CH₃. In certain embodiments, R⁹ is optionally substituted —(CH₂)—CH₃. In certain embodiments, R⁹ is optionally substituted —(CH₂)₈CH₃. In certain embodiments, R⁹ is optionally substituted —(CH₂)₉CH₃.

In certain embodiments, R⁹ is optionally substituted C₁-C₁₄ aliphatic-R¹¹. In certain embodiments, R⁹ is optionally substituted C₁-C₁₄ alkylene-R¹¹. In certain embodiments, R⁹ is optionally substituted C₁-C₁₄ branched alkylene-R¹¹. In certain embodiments, R⁹ is optionally substituted C₁-C₁₄ straight chain alkylene-R¹¹. In certain embodiments, R⁹ is optionally substituted C₁-C₁₄ alkenylene-R¹¹. In certain embodiments, R⁹ is optionally substituted C₁-C₁₄ branched alkenylene-R¹¹. In certain embodiments, R⁹ is optionally substituted C₁-C₁₄ straight chain alkenylene-R¹¹. In certain embodiments, R⁹ is optionally substituted C₁-C₅ alkylene-R¹¹. In certain embodiments, R⁹ is optionally substituted —(CH₂)—R¹¹. In certain embodiments, R⁹ is optionally substituted —(CH₂)₂—R¹¹. In certain embodiments, R⁹ is optionally substituted —(CH₂)₃—R¹¹. In certain embodiments, Rois optionally substituted —(CH₂)₄—R¹¹. In certain embodiments, R⁹ is optionally substituted —(CH₂)₅—R¹¹.

In certain embodiments, R⁸ and R⁹ are selected from

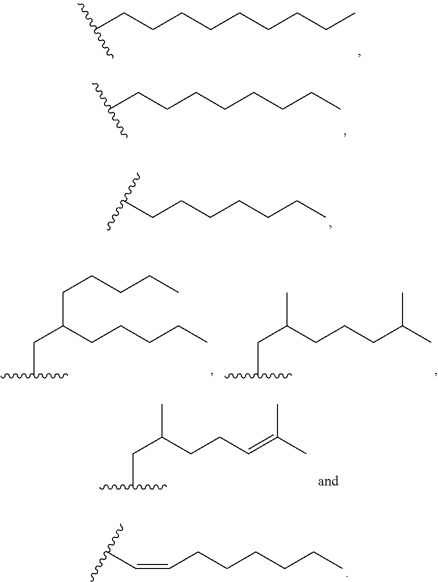

In certain embodiments, R⁸ and R⁹ are selected from

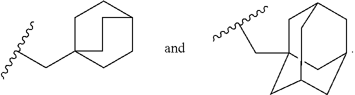 [SMILES: *CC12CCC(CC1)CC2 ; *CC12CC3CC(CC(C3)C1)C2]

R¹⁰ and R¹¹

As disclosed in Formula (CC), in certain embodiments, each R¹⁰ and R¹¹ are an optionally substituted bridged bicyclic or multicyclic C₅-C₁₂ cycloalkylenyl, or two R¹⁰ or two R¹¹ taken together form an optionally substituted bridged bicyclic or multicyclic C₅-C₁₂ cycloalkylenyl.

In certain embodiments, each R¹⁰ and R¹¹ are the same. In certain embodiments, each R¹⁰ and R¹¹ are different.

In some embodiments, each R¹⁰ and R¹¹ is independently an optionally substituted cyclic, bicyclic, bridged bicyclic, multicyclic or bridged multicyclic C₄-C₁₄ cycloalkyl or optionally substituted cyclic, bicyclic, bridged bicyclic, multicyclic or bridged multicyclic 4-14 membered heterocyclyl, or two R¹⁰ or two R¹¹ taken together form an optionally substituted bridged bicyclic or multicyclic C₄-C₁₄ cycloalkyl or optionally substituted bridged bicyclic or multicyclic 4-14 membered heterocyclyl.

In certain embodiments, each R¹⁰ is an optionally substituted bridged bicyclic C₅-C₁₂ cycloalkylenyl. In certain embodiments, each R¹⁰ is an optionally substituted bridged multicyclic C₅-C₁₂ cycloalkylenyl. In certain embodiments, each R¹¹ is an optionally substituted bridged bicyclic C₅-C₁₂ cycloalkylenyl. In certain embodiments, each R¹¹ is an optionally substituted bridged multicyclic C₅-C₁₂ cycloalkylenyl. In certain embodiments, the optionally substituted bridged bicyclic or multicyclic C₅-C₁₂ cycloalkylenyl is selected from adamantyl, bicyclo[2.2.2]octyl, cubanyl, bicyclo[1.1.1]pentyl, bicyclo[2.2.1]heptyl, bicyclo[3.1.1]heptyl, and bicyclo[3.2.1]octyl. In certain embodiments, the optionally substituted bridged bicyclic or multicyclic C₅-C₁₂ cycloalkylenyl is selected from:

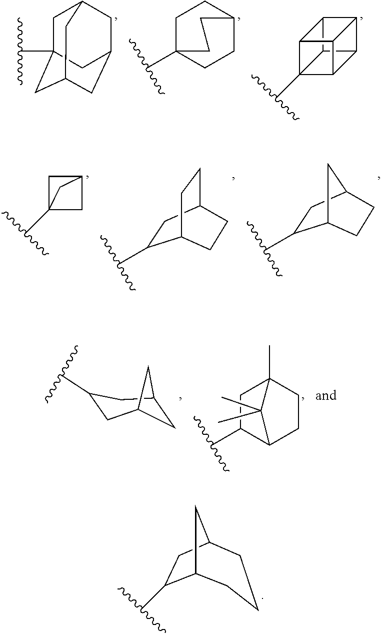

In certain embodiments, the substituted bridged bicyclic or multicyclic C₅-C₁₂ cycloalkylenyl is a structure selected from

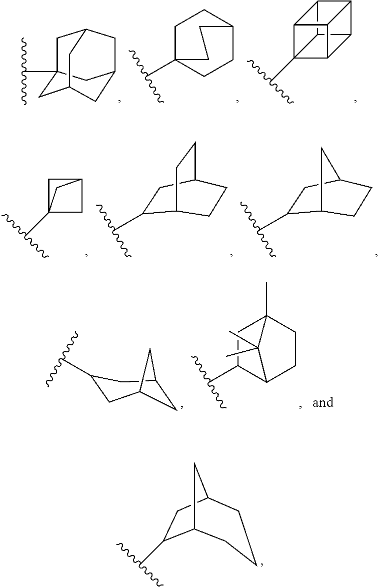

wherein one or more C—H bonds are substituted.

In certain embodiments, two R¹⁰ taken together form an optionally substituted bridged bicyclic or multicyclic C₅-C₁₂ cycloalkylenyl. In certain embodiments, two R¹¹ taken together form an optionally substituted bridged bicyclic or multicyclic C₅-C₁₂ cycloalkylenyl. In certain embodiments, the optionally substituted bridged bicyclic or multicyclic C₅-C₁₂ cycloalkylenyl is selected from:

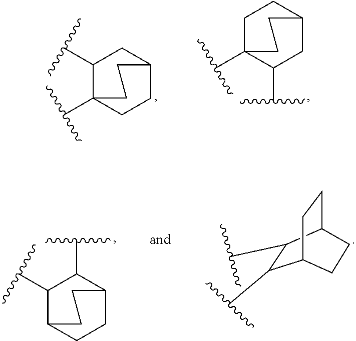 [SMILES: *C1CC2CCC1(*)CC2 ; *C1CC2CCC1(*)CC2 ; *C1C2CCC(CC2)C1* ; *C1C2CCC(CC2)C1*]

In some embodiments, Lipids of the Present Disclosure are selected from any lipid in Table (X) below or a pharmaceutically acceptable salt thereof:

TABLE (X)
Non-Limiting Examples of Ionizable Lipids of the Present Disclosure

| Cmpd | Structure |
|---|---|
| CC-1 | 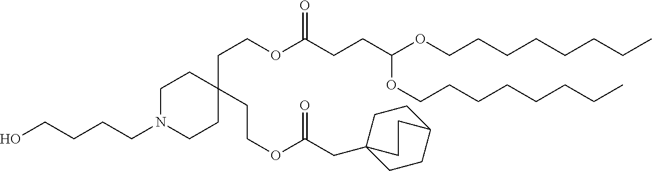 [SMILES: CCCCCCCCOC(CCC(=O)OCCC1(CCOC(=O)CC23CCC(CC2)CC3)CCN(CCCCO)CC1)OCCCCCCCC] |
| CC-2 | 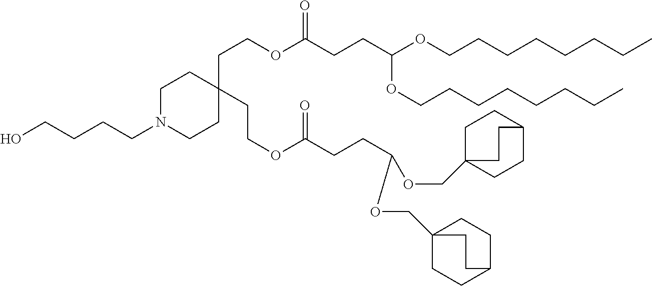 [SMILES: CCCCCCCCOC(CCC(=O)OCCC1(CCOC(=O)CCC(OCC23CCC(CC2)CC3)OCC23CCC(CC2)CC3)CCN(CCCCO)CC1)OCCCCCCCC] |
| CC-3 | 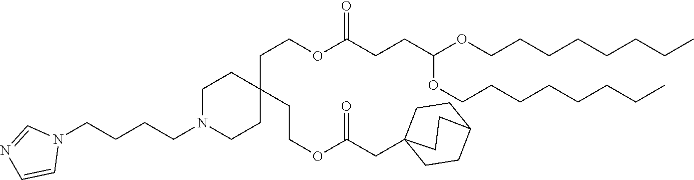 [SMILES: CCCCCCCCOC(CCC(=O)OCCC1(CCOC(=O)CC23CCC(CC2)CC3)CCN(CCCCn2ccnc2)CC1)OCCCCCCCC] |
| CC-4 | 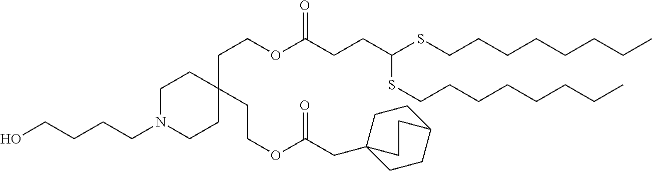 [SMILES: CCCCCCCCSC(CCC(=O)OCCC1(CCOC(=O)CC23CCC(CC2)CC3)CCN(CCCCO)CC1)SCCCCCCCC] |
| CC-5 | 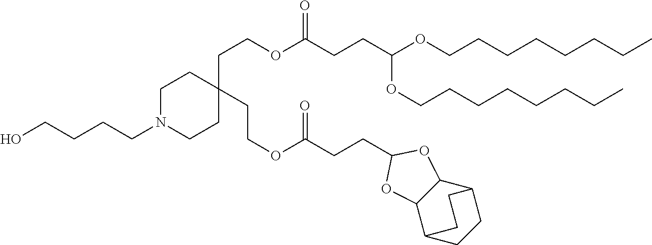 [SMILES: CCCCCCCCOC(CCC(=O)OCCC1(CCOC(=O)CCC2OC3C4CCC(CC4)C3O2)CCN(CCCCO)CC1)OCCCCCCCC] |
| CC-6 | 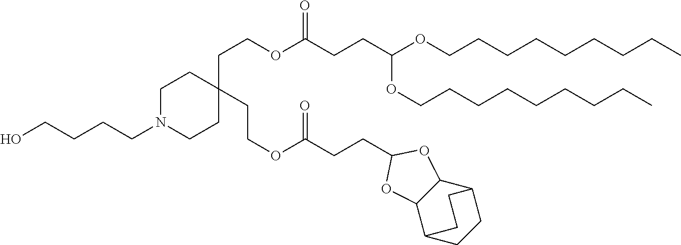 [SMILES: CCCCCCCCCOC(CCC(=O)OCCC1(CCOC(=O)CCC2OC3C4CCC(CC4)C3O2)CCN(CCCCO)CC1)OCCCCCCCCC] |
| CC-7 | 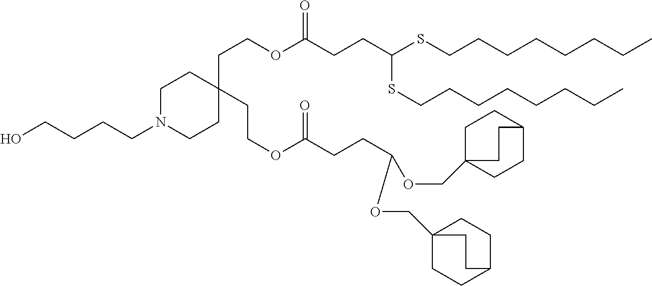 [SMILES: CCCCCCCCSC(CCC(=O)OCCC1(CCOC(=O)CCC(OCC23CCC(CC2)CC3)OCC23CCC(CC2)CC3)CCN(CCCCO)CC1)SCCCCCCCC] |
| CC-8 | 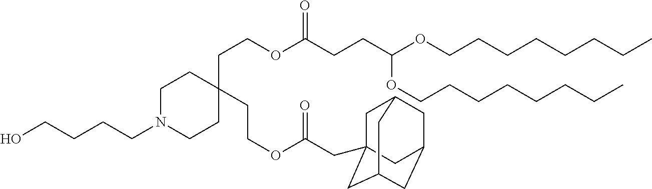 [SMILES: CCCCCCCCOC(CCC(=O)OCCC1(CCOC(=O)CC23CC4CC(CC(C4)C2)C3)CCN(CCCCO)CC1)OCCCCCCCC] |
| CC-9 | 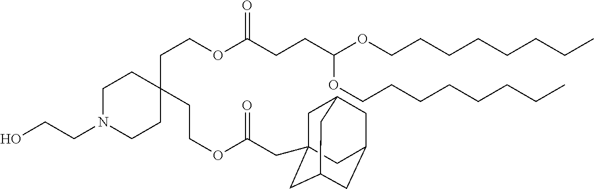 [SMILES: CCCCCCCCOC(CCC(=O)OCCC1(CCOC(=O)CC23CC4CC(CC(C4)C2)C3)CCN(CCO)CC1)OCCCCCCCC] |
| CC-10 | 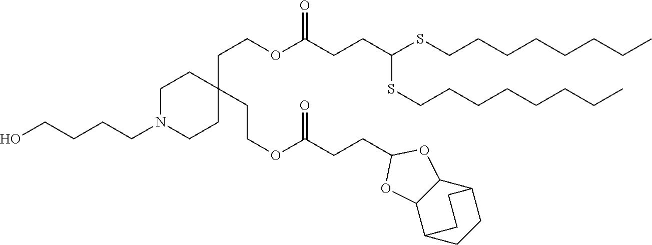 [SMILES: CCCCCCCCSC(CCC(=O)OCCC1(CCOC(=O)CCC2OC3C4CCC(CC4)C3O2)CCN(CCCCO)CC1)SCCCCCCCC] |
| CC-11 | 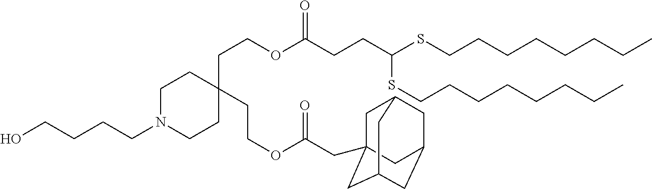 [SMILES: CCCCCCCCSC(CCC(=O)OCCC1(CCOC(=O)CC23CC4CC(CC(C4)C2)C3)CCN(CCCCO)CC1)SCCCCCCCC] |
| CC-12 | 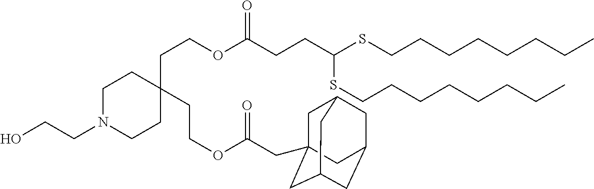 [SMILES: CCCCCCCCSC(CCC(=O)OCCC1(CCOC(=O)CC23CC4CC(CC(C4)C2)C3)CCN(CCO)CC1)SCCCCCCCC] |
| CC-13 | 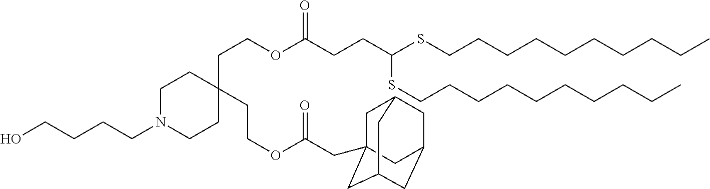 [SMILES: CCCCCCCCCCSC(CCC(=O)OCCC1(CCOC(=O)CC23CC4CC(CC(C4)C2)C3)CCN(CCCCO)CC1)SCCCCCCCCCC] |
| CC-14 | 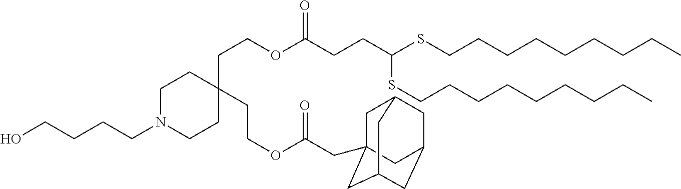 [SMILES: CCCCCCCCCSC(CCC(=O)OCCC1(CCOC(=O)CC23CC4CC(CC(C4)C2)C3)CCN(CCCCO)CC1)SCCCCCCCCC] |
| CC-15 | 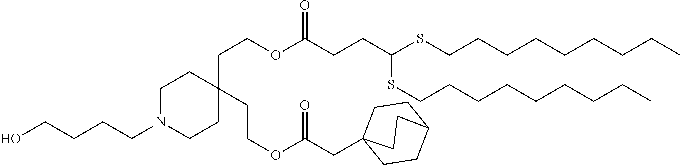 [SMILES: CCCCCCCCCSC(CCC(=O)OCCC1(CCOC(=O)CC23CCC(CC2)CC3)CCN(CCCCO)CC1)SCCCCCCCCC] |
| CC-16 | 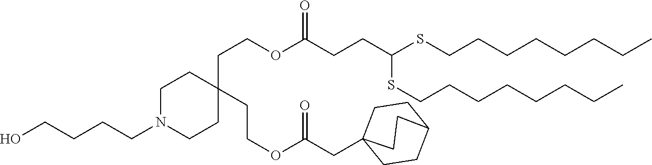 [SMILES: CCCCCCCCSC(CCC(=O)OCCC1(CCOC(=O)CC23CCC(CC2)CC3)CCN(CCCCO)CC1)SCCCCCCCC] |
| CC-17 | 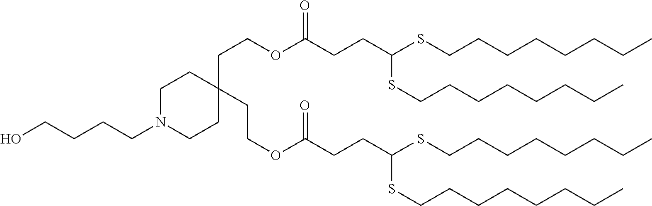 [SMILES: CCCCCCCCSC(CCC(=O)OCCC1(CCOC(=O)CCC(SCCCCCCCC)SCCCCCCCC)CCN(CCCCO)CC1)SCCCCCCCC] |
| CC-18 | 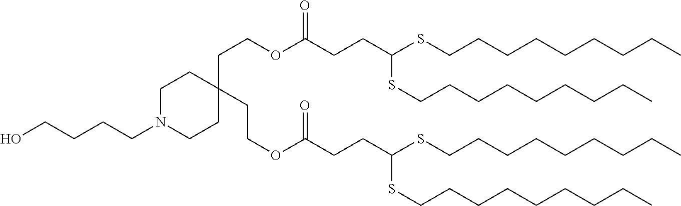 [SMILES: CCCCCCCCCSC(CCC(=O)OCCC1(CCOC(=O)CCC(SCCCCCCCCC)SCCCCCCCCC)CCN(CCCCO)CC1)SCCCCCCCCC] |
| CC-19 | 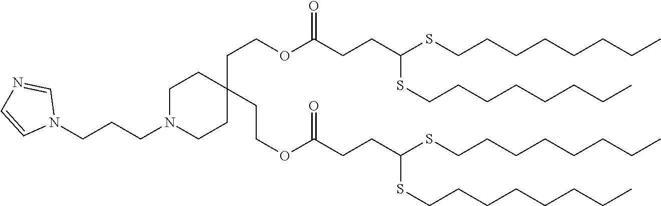 [SMILES: CCCCCCCCSC(CCC(=O)OCCC1(CCOC(=O)CCC(SCCCCCCCC)SCCCCCCCC)CCN(CCCn2ccnc2)CC1)SCCCCCCCC] |
| CC-20 | 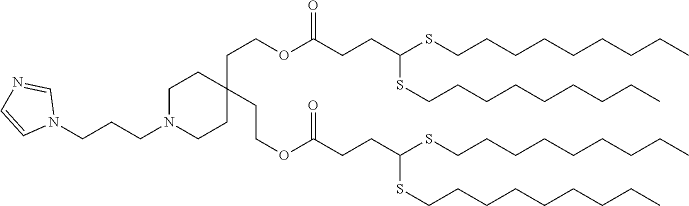 [SMILES: CCCCCCCCCSC(CCC(=O)OCCC1(CCOC(=O)CCC(SCCCCCCCCC)SCCCCCCCCC)CCN(CCCn2ccnc2)CC1)SCCCCCCCCC] |
| CC-21 | 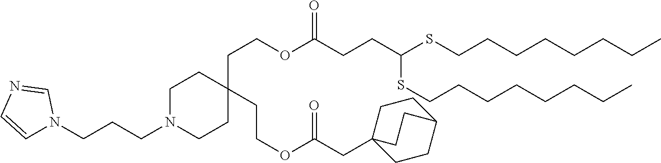 [SMILES: CCCCCCCCSC(CCC(=O)OCCC1(CCOC(=O)CC23CCC(CC2)CC3)CCN(CCCn2ccnc2)CC1)SCCCCCCCC] |
| CC-22 | 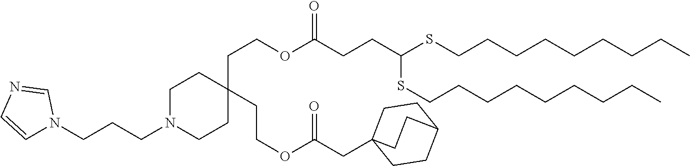 [SMILES: CCCCCCCCCSC(CCC(=O)OCCC1(CCOC(=O)CC23CCC(CC2)CC3)CCN(CCCn2ccnc2)CC1)SCCCCCCCCC] |
| CC-23 | 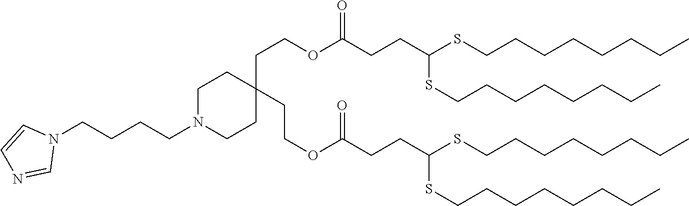 [SMILES: CCCCCCCCSC(CCC(=O)OCCC1(CCOC(=O)CCC(SCCCCCCCC)SCCCCCCCC)CCN(CCCCn2ccnc2)CC1)SCCCCCCCC] |
| CC-24 | 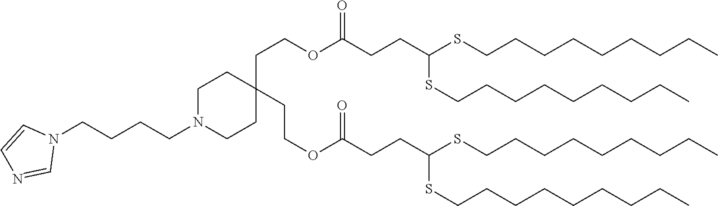 [SMILES: CCCCCCCCCSC(CCC(=O)OCCC1(CCOC(=O)CCC(SCCCCCCCCC)SCCCCCCCCC)CCN(CCCCn2ccnc2)CC1)SCCCCCCCCC] |
| CC-25 | 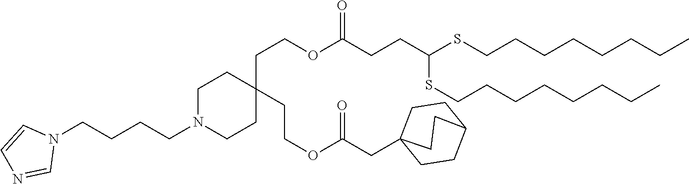 [SMILES: CCCCCCCCSC(CCC(=O)OCCC1(CCOC(=O)CC23CCC(CC2)CC3)CCN(CCCCn2ccnc2)CC1)SCCCCCCCC] |
| CC-26 | 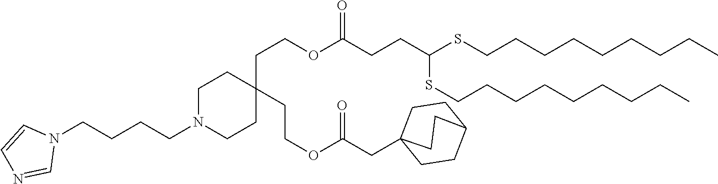 [SMILES: CCCCCCCCCSC(CCC(=O)OCCC1(CCOC(=O)CC23CCC(CC2)CC3)CCN(CCCCn2ccnc2)CC1)SCCCCCCCCC] |
| CC-27 | 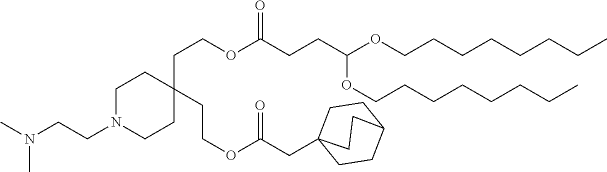 [SMILES: CCCCCCCCOC(CCC(=O)OCCC1(CCOC(=O)CC23CCC(CC2)CC3)CCN(CCN(C)C)CC1)OCCCCCCCC] |
| CC-100 | 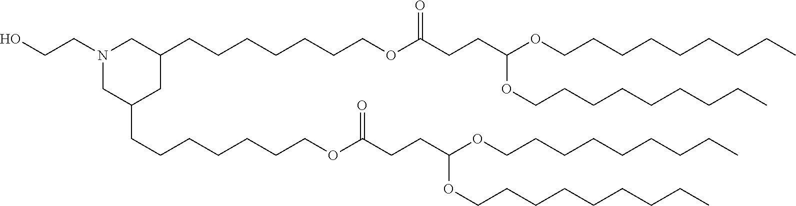 [SMILES: CCCCCCCCCOC(CCC(=O)OCCCCCCCC1CC(CCCCCCCOC(=O)CCC(OCCCCCCCCC)OCCCCCCCCC)CN(CCO)C1)OCCCCCCCCC] |
| CC-101 | 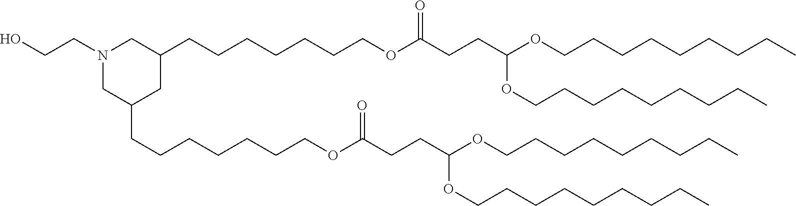 [SMILES: CCCCCCCCCOC(CCC(=O)OCCCCCCCC1CC(CCCCCCCOC(=O)CCC(OCCCCCCCCC)OCCCCCCCCC)CN(CCO)C1)OCCCCCCCCC] |
| CC-102 | 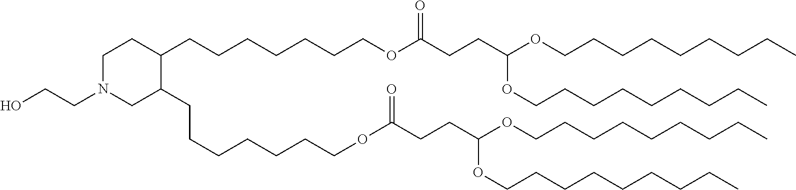 [SMILES: CCCCCCCCCOC(CCC(=O)OCCCCCCCC1CCN(CCO)CC1CCCCCCCOC(=O)CCC(OCCCCCCCCC)OCCCCCCCCC)OCCCCCCCCC] |
| CC-103 | 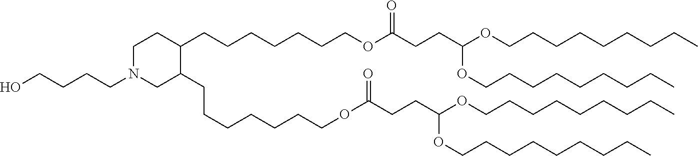 [SMILES: CCCCCCCCCOC(CCC(=O)OCCCCCCCC1CCN(CCCCO)CC1CCCCCCCOC(=O)CCC(OCCCCCCCCC)OCCCCCCCCC)OCCCCCCCCC] |
| CC-104 | 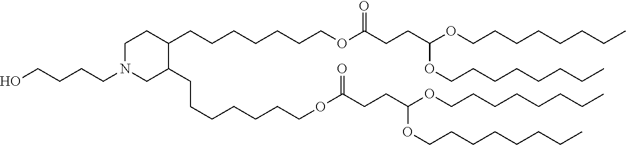 [SMILES: CCCCCCCCOC(CCC(=O)OCCCCCCCC1CCN(CCCCO)CC1CCCCCCCOC(=O)CCC(OCCCCCCCC)OCCCCCCCC)OCCCCCCCC] |
| CC-105 | 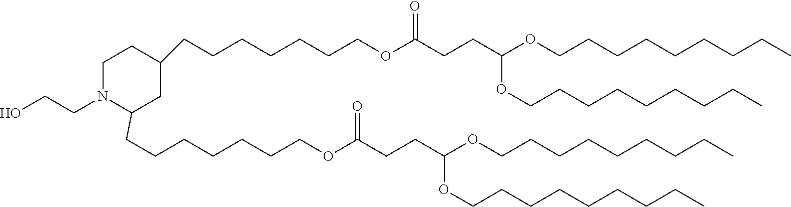 [SMILES: CCCCCCCCCOC(CCC(=O)OCCCCCCCC1CCN(CCO)C(CCCCCCCOC(=O)CCC(OCCCCCCCCC)OCCCCCCCCC)C1)OCCCCCCCCC] |
| CC-106 | 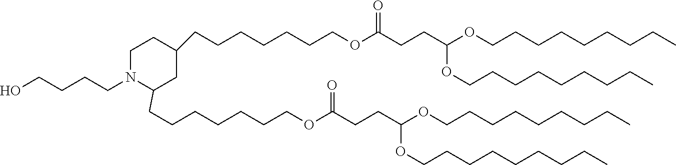 [SMILES: CCCCCCCCCOC(CCC(=O)OCCCCCCCC1CCN(CCCCO)C(CCCCCCCOC(=O)CCC(OCCCCCCCCC)OCCCCCCCCC)C1)OCCCCCCCCC] |
| CC-107 | 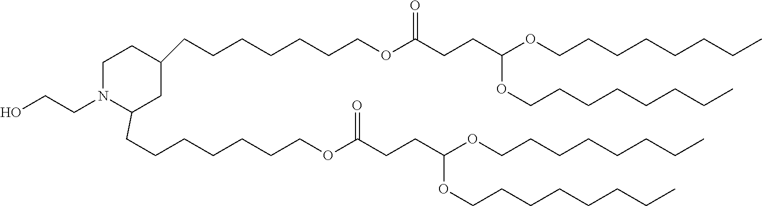 [SMILES: CCCCCCCCOC(CCC(=O)OCCCCCCCC1CCN(CCO)C(CCCCCCCOC(=O)CCC(OCCCCCCCC)OCCCCCCCC)C1)OCCCCCCCC] |
| C-108 | 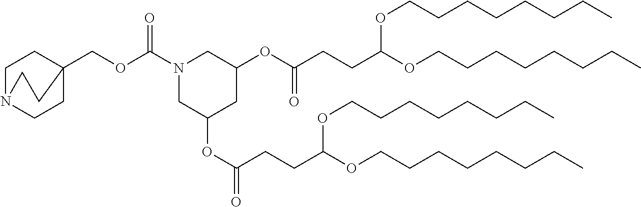 [SMILES: CCCCCCCCOC(CCC(=O)OC1CC(OC(=O)CCC(OCCCCCCCC)OCCCCCCCC)CN(C(=O)OCC23CCN(CC2)CC3)C1)OCCCCCCCC] |

ii. Structural Lipids

In some embodiments, an LNP comprises a structural lipid. Structural lipids can be selected from the group consisting of, but are not limited to, cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupcol, oleanolic acid, beta-sitosterol-acetate and mixtures thereof. In some embodiments, the structural lipid is cholesterol. In some embodiments, the structural lipid is a cholesterol analogue disclosed by Patel, et al., Nat Commun., 11, 983 (2020), which is incorporated herein by reference in its entirety. In some embodiments, the structural lipid includes cholesterol and a corticosteroid (such as prednisolone, dexamethasone, prednisone, and hydrocortisone), or any combinations thereof. In some embodiments, a structural lipid is described in international patent application WO2019152557A1, which is incorporated herein by reference in its entirety.

In some embodiments, a structural lipid is a cholesterol analog. Using a cholesterol analog may enhance endosomal escape as described in Patel et al., Naturally-occurring cholesterol analogues in lipid nanoparticles induce polymorphic shape and enhance intracellular delivery of mRNA, Nature Communications (2020), which is incorporated herein by reference.

In some embodiments, a structural lipid is a phytosterol. Using a phytosterol may enhance endosomal escape as described in Herrera et al., Illuminating endosomal escape of polymorphic lipid nanoparticles that boost mRNA delivery, Biomaterials Science (2020), which is incorporated herein by reference.

In some embodiments, a structural lipid contains plant sterol mimetics for enhanced endosomal release.

iii. PEGylated Lipids

A PEGylated lipid is a lipid modified with polyethylene glycol. The term “PEGylated lipid” is used interchangeably herein with the shortened term “PEG lipid”.

In some embodiments, an LNP comprises one, two or more PEGylated lipid or PEG-modified lipid. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the PEGylated lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-CDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG (2k)-DMG, PEG DSPE C18, PEG DMPE C14, PEG DLPE C12, PEG Click DMG C14, PEG Click C12, PEG Click C10, N (Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.

In some embodiments, the LNP comprises a PEGylated lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; or WO 2019/089828A1, each of which is incorporated by reference herein in their entirety.

In some embodiments, the LNP comprises a PEGylated lipid substitute in place of the PEGylated lipid. All embodiments disclosed herein that contemplate a PEGylated lipid should be understood to also apply to PEGylated lipid substitutes. In some embodiments, the LNP comprises a polysarcosine-lipid conjugate, such as those disclosed in US 2022/0001025 A1, which is incorporated by reference herein in its entirety. In some embodiments the LNP comprises a polyoxazoline-lipid conjugate, such as those disclosed in US 2022/0249695 A1, which is incorporated by reference herein in its entirety.

In some embodiments, the LNP comprises a PEGylated lipid disclosed and described in PCT Application WO2024044728A1, which is incorporated by reference herein, in its entirety. In certain embodiments, the PEGylated lipid is a lipid of any one of formulas PL-I′, PL-I″, PL-I, PL-Ia, PL-Ib, PL-Iaa, PL-Iab, PL-Iac, PL-Iad, PL-Iae, PL-Iaf, PL-Iag, PL-Iah, PL-Iba, PL-Ibb, PL-Ibc, PL-Ibd, PL-Ibe, PL-Ibf, PL-Ibg, PL-Ibh, PL-Ica, PL-Icb, PL-Icc, PL-Icd, PL-Id PL-Ie, PL-If, PL-Ig, PL-Ih, PL-Ii, PL-Iha, PL-Ihb, PL-Ihc, PL-Ihd, PL-Iia, PL-Iib, PL-Iic, PL-Iid, PL-Ij, PL-Ik, L-Il, PL-Im, PL-In, PL-Io, PL-Ip, PL-Iq, PL-Ioa, PL-Iob, PL-Ioc, PL-Iod, PL-Ioe, PL-Iof, PL-Iog, PL-Ioh, PL-Ipa, PL-Ipb, PL-Ipc, PL-Ipd, PL-Ipe, PL-Ipf, PL-Ipg, PL-Iph, PL-Iqa, PL-Iqb, PL-Iqc, PL-Iqd, PL-Ir, PL-Is, PL-It, PL-Iu, PL-Iv, PL-Iw, PL-Iva, PL-Ivb, PL-Ivc, PL-Ivd, PL-Iwa, PL-Iwb, PL-Iwc, PL-Iwd, PL-Ix, PL-Ixx, PL-Iy, PL-Iyy, PL-Iyyy, PL-Iz, PL-Izz, PL-Izzz, PL-II′, PL-II″, PL-II, PL-IIc, PL-IId, PL-IIe, PL-IIf, PL-IIg, PL-IIh, PL-IIa, PL-IIb, PL-IIk, PL-IIm or PL-IIn.

In some embodiments, the PEGylated lipid is a compound of formula PL-I′:

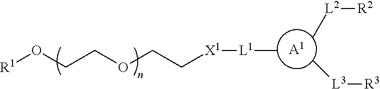

or a pharmaceutically acceptable salt thereof, wherein:
A¹ is a saturated 5-6 membered carbocyclic ring or a saturated 5-6 membered heterocyclic ring containing 1 or 2 heteroatoms independently selected from nitrogen, oxygen, and sulfur, wherein the carbocyclic ring and heterocyclic ring are substituted with t occurrences of R⁴;
X¹ is —N(H)—, —N(C₁₋₆ alkyl)-, —C₁₋₆ aliphatic-N(H)—, —C₁₋₆ aliphatic-N(C₁₋₆ alkyl)-, —O— or —C₁₋₆ aliphatic-O—;
L¹ is —C(O)(C₁₋₆ aliphatic) C(O)—N(R)—, —C(O)(C₁₋₆ aliphatic)-N(R)C(O)—, —C(O)(C₁₋₆ aliphatic) C(O)O—, —C(O)(C₁₋₆ aliphatic) C(O)—, —C(O)(C₁₋₆ aliphatic) C(O)OCH₂—, —C(O)(C₁₋₆ aliphatic)-, —C(O)(C₁₋₆ aliphatic)-N(R)—, or —C(O)—;
L² and L³ are independently a covalent bond or C₁₋₆ alkylene wherein one methylene unit of the C₁₋₆ alkylene is optionally replaced with —O—, —NR—, —S—, —S—S—, —S(O)—, —S(O)₂—, —C(O)—, —C(O)O—, —OC(O)—, —OC(O)O—, —OC(O)N(R)—, —N(R)C(O)O—, —C(O)N(R)—, —N(R)C(O)—, —N(R)C(O)N(R)—, —C(R⁵)═N—, or —C(R⁵)═N—O—;
R¹ is H, C₁₋₆ alkyl, —(C₁₋₆ alkyl)-N₃, —(C₁₋₆ alkyl)-SH, or C₃₋₈ alkynyl;
R² and R³ are independently a straight or branched C₆₋₃₀ alkyl, straight or branched C₆₋₃₀ alkenyl, or straight or branched C₆₋₃₀ alkynyl; wherein 1, 2, or 3 methylene units are independently and optionally replaced by a saturated or partially unsaturated C₃₋₆ carbocyclic ring or phenylene; wherein the alkyl, alkenyl, and alkynyl and any carbocyclic ring or phenylene is substituted with m instances of R^{x};
R⁴ is C₁₋₄ alkyl;
R⁵ is C₁₋₆ alkyl or C₂₋₁₄ alkenyl;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, or sulfur;
each R^{X} is independently halogen, —CN, —OR, —SR, —C(O)R, —C(O)OR, or —OC(O)OR;
n is an integer from 10-75, inclusive;
m is 0, 1, 2, 3, or 4; and
t is 0, 1, or 2.

In some embodiments, the PEGylated lipid is a compound of formula PL-II′:

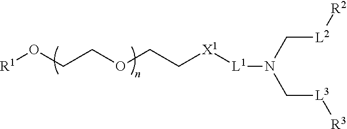

or a pharmaceutically acceptable salt thereof, wherein:
X¹ is —N(H)—, —N(C₁₋₆ alkyl)-, —C₁₋₆ aliphatic-N(H)—, —C₁₋₆ aliphatic-N(C₁₋₆ alkyl)-, —O— or —C₁₋₆ aliphatic-O—;
L¹ is —C(O)(C₁₋₆ aliphatic) C(O)—, —C(O)(C₁₋₆ aliphatic)-, or —C(O)—;
L² and L³ are a covalent bond or C₁₋₆ alkylene wherein one methylene unit of the C₁₋₆ alkylene is optionally replaced with —O—, —NR—, —S—, —S—S—, —S(O)—, —S(O)₂—, —C(O)—, —C(O)O—, —OC(O)—, —OC(O)O—, —OC(O)N(R)—, —N(R)C(O)O—, —C(O)N(R)—, —N(R)C(O)—, —N(R)C(O)N(R)—, —C(R⁶)═N—, or —C(R⁶)═N—O—;
R¹ is H, C₁₋₆ alkyl, —(C₁₋₆ alkyl)-N₃, —(C₁₋₆ alkyl)-SH, or C₃₋₈ alkynyl;
R² and R³ are independently straight or branched C₆₋₃₀ alkyl, straight or branched C₆₋₃₀ alkenyl, or straight or branched C₆₋₃₀ alkynyl; wherein 1, 2, or 3 methylene units are independently and optionally replaced by a saturated or partially unsaturated C₃₋₆ carbocyclic ring or phenylene; wherein the alkyl, alkenyl, and alkynyl and any carbocyclic ring or phenylene is substituted with m instances of R^{x};
R⁶ is C₁₋₆ alkyl or C₂₋₁₄ alkenyl;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, and sulfur, a 5-6 membered monocyclic heteroaromatic ring having 1-4 heteroatoms independently selected from nitrogen, oxygen, and sulfur, or an 8-10 membered bicyclic heteroaromatic ring having 1-5 heteroatoms independently selected from nitrogen, oxygen, or sulfur;
each R^{x} is independently halogen, —CN, —OR, —SR, —C(O)R, —C(O)OR, or OC(O)OR;
n is an integer from 10-75, inclusive; and
m is 0, 1, 2, 3, or 4.

In some embodiments, the PEGylated lipid compound is one of those shown in Table XI, or a pharmaceutically acceptable salt thereof.

TABLE XI
Exemplary PEGylated Compounds

| Compound | |
|---|---|
| No. | Structure |
| PL-1 | 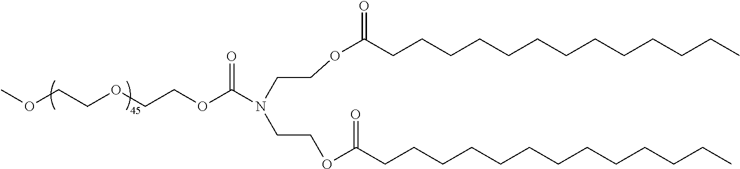 |
| PL-1A | 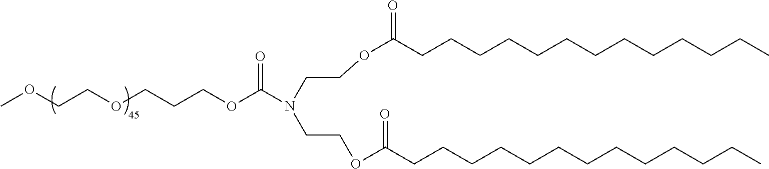 |
| PL-2 | 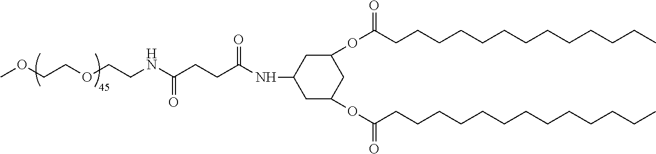 |
| PL-2A | 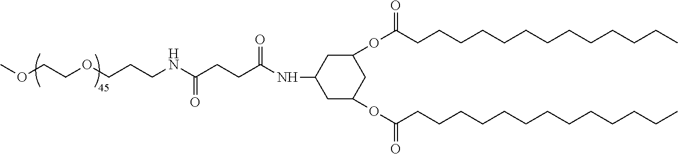 |
| PL-3 | 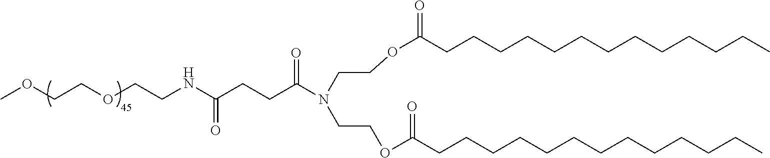 |
| PL-3A | 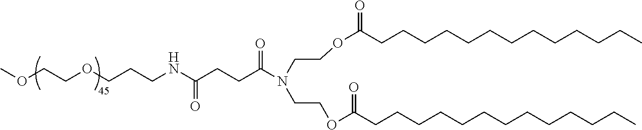 |
| PL-4 | 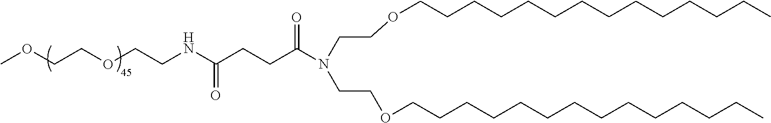 |
| PL-4A | 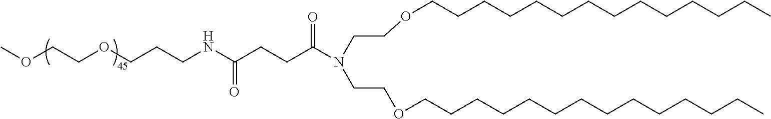 |
| PL-5 | 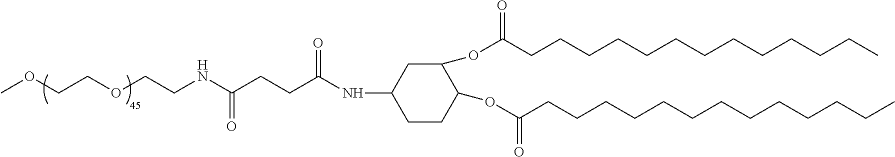 |
| PL-5A | 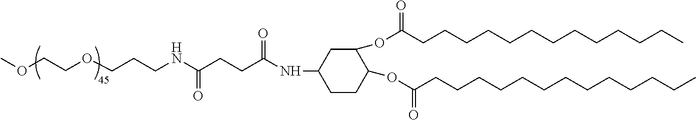 |
| PL-6 | 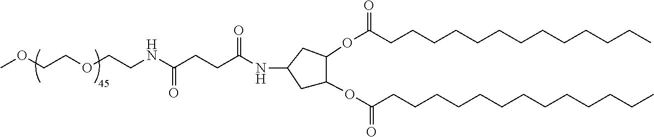 |
| PL-6A | 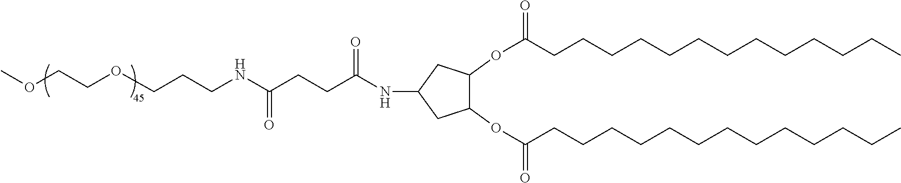 |
| PL-7 | 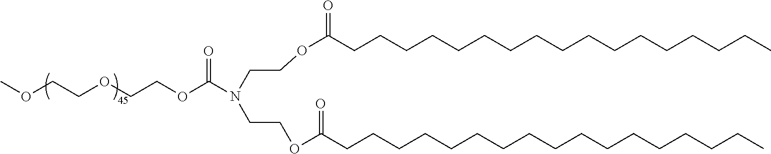 |
| PL-7A | 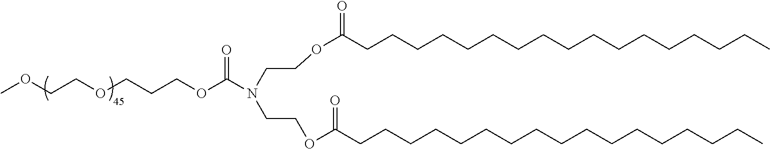 |
| PL-8 | 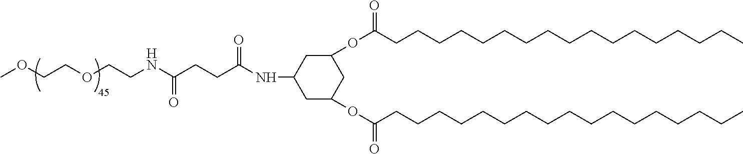 |
| PL-8A | 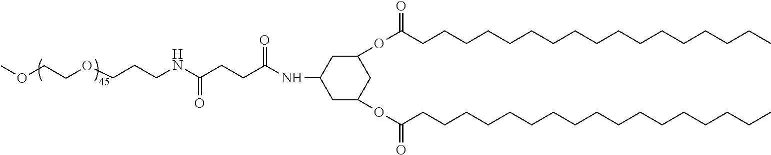 |
| PL-9 | 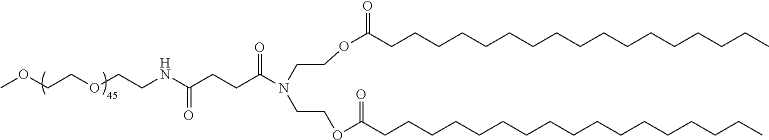 |
| PL-9A | 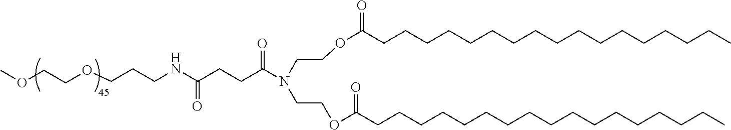 |
| PL-10 | 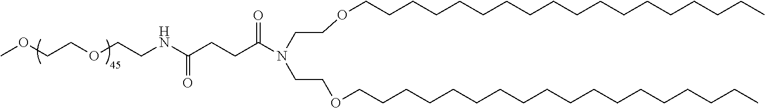 |
| PL-10A | 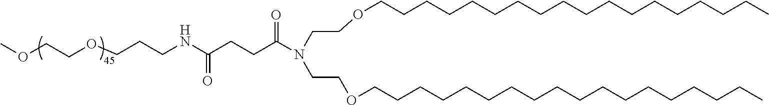 |
| PL-11 | 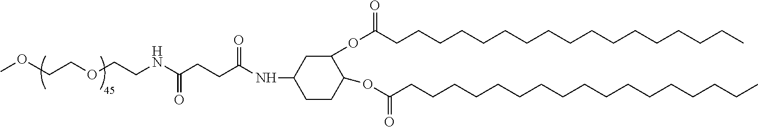 |
| PL-11A | 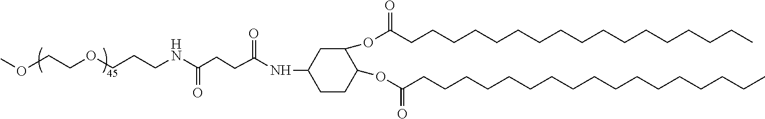 |
| PL-12 | 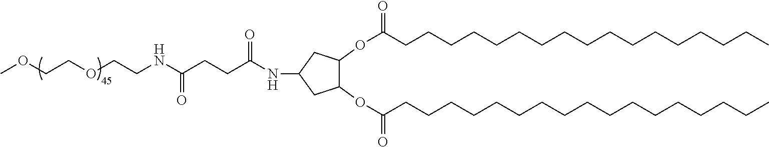 |
| PL-12A | 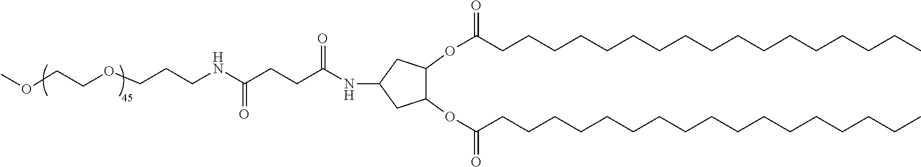 |
| PL-12B | 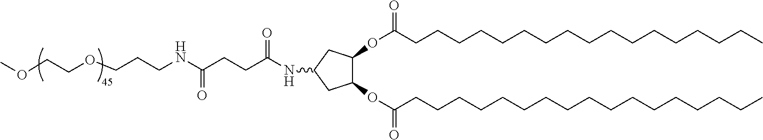 |
| PL-13 | 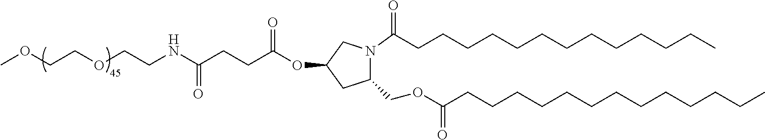 |
| PL-13A | 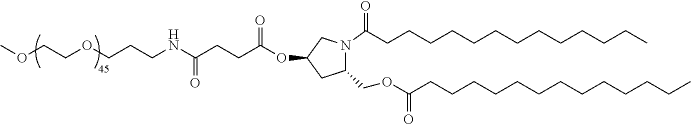 |
| PL-14 | 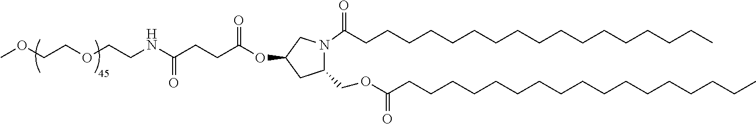 |
| PL-14A | 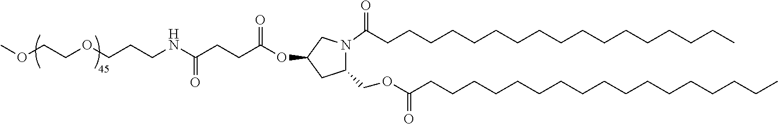 |
| PL-15 | 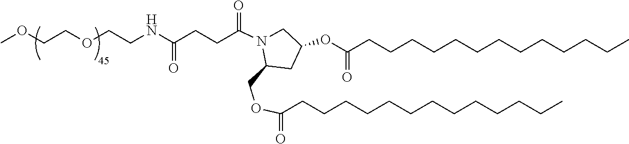 |
| PL-15A | 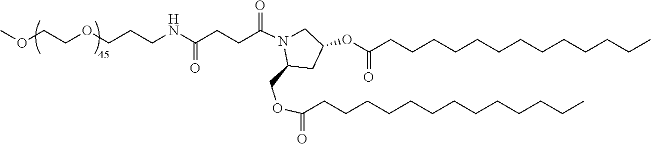 |
| PL-16 | 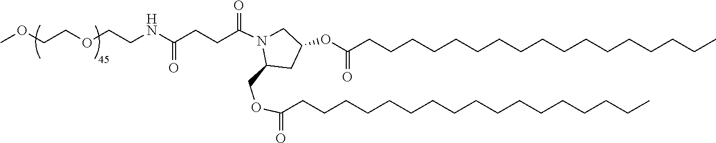 |
| PL-16A | 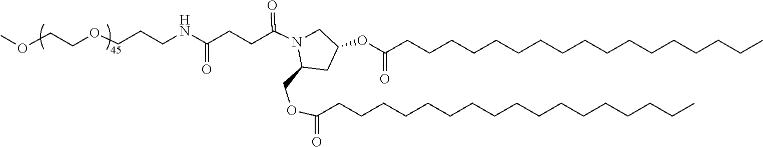 |

iv. Phospholipids

In some embodiments, an LNP of the present disclosure comprises a phospholipid. Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium(S)-2-ammonio-3-((((R)-2-(oleoyloxy)-3-(stearoyloxy)propoxy)oxidophosphoryl)oxy)propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin. In some embodiments, an LNP includes DSPC. In certain embodiments, an LNP includes DOPE. In some embodiments, an LNP includes both DSPC and DOPE.

In some embodiments, an LNP comprises a phospholipid selected from 1-pentadecanoyl-2-oleoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-palmitoyl-2-oleoyl-glycero-3-phosphocholine, 1-palmitoyl-2-linoleoyl-sn-glycero-3-phosphocholine, 1-palmitoyl-2-arachidonoyl-sn-glycero-3-phosphocholine, 1-palmitoyl-2-docosahexaenoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-stearoyl-2-oleoyl-sn-glycero-3-phosphocholine, 1-stearoyl-2-linoleoyl-sn-glycero-3-phosphocholine, 1-stearoyl-2-arachidonoyl-sn-glycero-3-phosphocholine, 1-stearoyl-2-docosahexaenoyl-sn-glycero-3-phosphocholine, 1-oleoyl-2-myristoyl-sn-glycero-3-phosphocholine, 1-oleoyl-2-palmitoyl-sn-glycero-3-phosphocholine, 1-oleoyl-2-stearoyl-sn-glycero-3-phosphocholine, 1-palmitoyl-2-acetyl-sn-glycero-3-phosphocholine, 1,2-diolcoyl-sn-glycero-3-phospho-(1′-myo-inositol-3′,4′-bisphosphate), 1,2-dioleoyl-sn-glycero-3-phospho-(1′-myo-inositol-3′,5′-bisphosphate), 1,2-dioleoyl-sn-glycero-3-phospho-(1′-myo-inositol-4′,5′-bisphosphate), 1,2-dioleoyl-sn-glycero-3-phospho-(1′-myo-inositol-3′,4′,5′-trisphosphate), 1,2-dioleoyl-sn-glycero-3-phospho-(1′-myo-inositol-3′-phosphate), 1,2-dioleoyl-sn-glycero-3-phospho-(1′-myo-inositol-4′-phosphate), 1,2-dioleoyl-sn-glycero-3-phospho-(1′-myo-inositol-5′-phosphate), 1,2-dioleoyl-sn-glycero-3-phospho-(1′-myo-inositol), 1,2-dioleoyl-sn-glycero-3-phospho-L-serine, and 1-(8Z-octadecenoyl)-2-palmitoyl-sn-glycero-3-phosphocholine.

In some embodiments, the LNP comprises a phospholipid selected from DSPS (Distearoylphosphatidylserine), DSPG (1,2-distearoyl-sn-glycero-3-phospho-(1′-rac-glycerol)), DSPA (1,2-Distearoyl-sn-glycero-3-phosphate), diPhyPC (1,2-diphytanoyl-sn-glycero-3-phosphocholine), diPhy-diether-PC (1,2-di-O-phytanyl-sn-glycero-3-phosphocholine), diPhyPE (1,2-diphytanoyl-sn-glycero-3-phosphoethanolamine), diPhy-diether-PE (1,2-di-O-phytanyl-sn-glycero-3-phosphoethanolamine), diPhyPS (1,2-diphytanoyl-sn-glycero-3-phospho-L-serine), diPhyPG (1,2-diphytanoyl-sn-glycero-3-phospho-(1′-rac-glycerol)), diPhyPA (1,2-diphytanoyl-sn-glycero-3-phosphate), Egg PA (L-α-phosphatidic acid), and Soy PA (L-α-phosphatidic acid).

In some embodiments, the LNP comprises a phospholipid selected from 18:1 (49-Cis) PE (DOPE), 18:0-18:1 PE (SOPE), C16-18:1 PE, 16:0-18:1 PE (POPE), 18:1 BMP (S,R), 18:0-18:1 PC (SOPC), 16:0-18:1 PC (POPC), 4ME 16:0 Diether PE (4Me), 18:1 (49-Trans) PE (DEPE), 16:1 PE (DPPE), and CL. In certain embodiments, the LNP comprises a phospholipid described or disclosed in Alvarez-Benedicto, et al. (Biomater. Sci., 2022, 10, 549) and Li, et al. (Asian Journal of Pharmaceutical Sciences, 2015, 10, 81-98).

In certain embodiments, the phospholipid is a sphingoid lipid or sphingolipid, such as, but not limited to sphingomyelin. As used herein, the terms “sphingoid lipid” and “sphingolipid” are meant to refer to a class of lipids containing a backbone comprising a sphingoid base. An exemplary sphingoid base is sphingosine. In certain embodiments, the LNP comprises a sphingolipid selected from Egg Sphingomyelin (Egg SM/ESM/(2S,3R,E)-3-hydroxy-2-palmitamidooctadec-4-en-1-yl (2-(trimethylammonio)ethyl)phosphate), Brain or Porcine Sphingomyelin (Brain SM/(2S,3R,E)-3-hydroxy-2-stearamidooctadec-4-en-1-yl (2-(trimethylammonio)ethyl)phosphate), Milk or Bovine Sphingomyelin (Milk SM/(2S,3R,E)-3-hydroxy-2-tricosanamidooctadec-4-en-1-yl (2-(trimethylammonio)ethyl)phosphate), 28:0 SM (N-octacosanoyl-D-erythro-sphingosylphosphorylcholine), 14:0 SM (N-myristoyl-D-erythro-sphingosylphosphorylcholine), 16:1 SM (N-palmitoleoyl-D-erythro-sphingosylphosphorylcholine), 12:0 Dihydro SM (N-lauroyl-D-erythro-sphinganylphosphorylcholine), Lyso SM (Sphingosylphosphorylcholine), Lyso SM (Sphingosylphosphorylcholine), Lyso SM (dihydro) (Sphinganine Phosphorylcholine), 24:1 SM (N-nervonoyl-D-erythro-sphingosylphosphorylcholine), 24:0 SM (N-lignoceroyl-D-erythro-sphingosylphosphorylcholine), 18:1 SM (N-oleoyl-D-erythro-sphingosylphosphorylcholine), 18:0 SM (N-stearoyl-D-erythro-sphingosylphosphorylcholine), 17:0 SM (N-heptadecanoyl-D-crythro-sphingosylphosphorylcholine), 16:0 SM (N-palmitoyl-D-erythro-sphingosylphosphorylcholine), 12:0 SM (N-lauroyl-D-erythro-sphingosylphosphorylcholine), 06:0 SM (N-hexanoyl-D-erythro-sphingosylphosphorylcholine), 02:0 SM (N-acetyl-D-erythro-sphingosylphosphorylcholine), 3-O-methyl Lyso SM (3-O-methyl-spingosylphosphorylcholine), 3-O-methyl-N-methyl Lyso SM (3-O-methyl-N-methyl-spingosylphosphorylcholine), and 3-N-methyl Lyso SM (3-N-methyl-spingosylphosphorylcholine).

In some embodiments, the LNP comprises a phospholipid comprising at least one constrained tail, such as those described by Gan, et al. (Bioeng Transl Med. 2020 September; 5(3):e10161.). In certain embodiments, the phospholipid is one selected from:

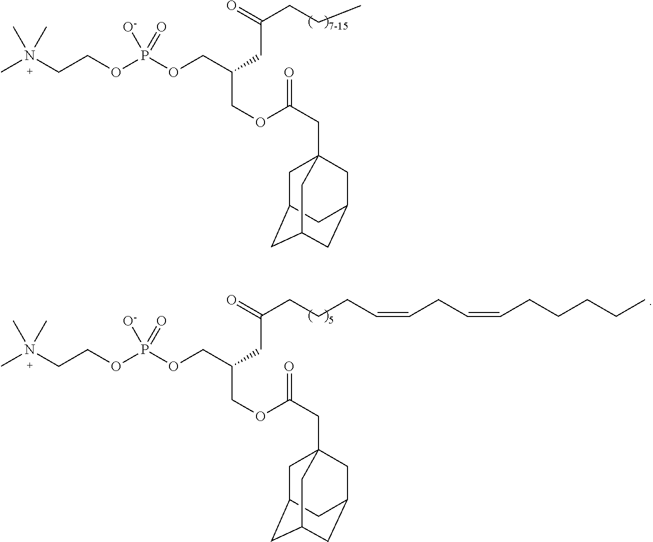

In some embodiments, the LNP comprises a phospholipid comprising a ceramide analogue having a triazole linkage, such as those described by Kim et al., Bioorg. Med. Chem. Lett., 17(16), 2007, 4584-4587.

In some embodiments, the LNP comprises a phospholipid disclosed in WO 2023/141470, which is incorporated by reference herein, in its entirety. In certain embodiments, the phospholipid is

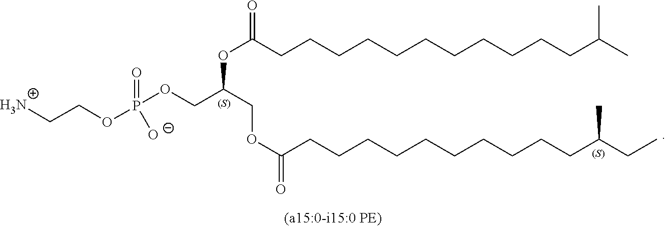

In some embodiments, the LNP comprises a phospholipid disclosed in WO2022040641A2, which is incorporated by reference herein, in its entirety.

In some embodiments, a phospholipid tail may be modified in order to promote endosomal escape as described in U.S. Application Publication 2021/0121411, which is incorporated herein by reference.

In some embodiments, the LNP comprises a phospholipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, phospholipids disclosed in US 2020/0121809 have the following structure:

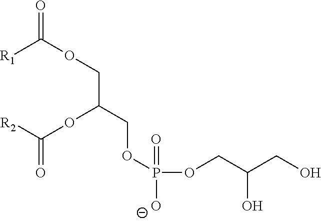

wherein R¹ and R² are each independently a branched or straight, saturated or unsaturated carbon chain (e.g., alkyl, alkenyl, alkynyl).
vi. Targeting Moieties

In some embodiments, the lipid nanoparticle further comprises a targeting moiety. The targeting moiety may be an antibody or a fragment thereof. The targeting moiety may be capable of binding to a target antigen. In certain embodiments, the lipid nanoparticle comprises more than one targeting moiety. In certain embodiments, the lipid nanoparticle comprises more than one targeting moiety, wherein the targeting moieties target at least two different receptors, and in some embodiments, the at least two different receptors are prevalent on different types of cells or tissues.

In some embodiments, the pharmaceutical composition comprises a targeting moiety that is operably connected to a lipid nanoparticle. In some embodiments, the targeting moiety is capable of binding to a target antigen. In some embodiments, the target antigen is expressed in a target organ. In some embodiments, the target antigen is expressed more in the target organ than it is in the liver.

In some embodiments, the targeting moiety is an antibody as described in WO2016189532A1, which is incorporated herein by reference. For example, in some embodiments, the targeted particles are conjugated to a specific anti-CD38 monoclonal antibody (mAb), which allows specific delivery of the siRNAs encapsulated within the particles at a greater percentage to B-cell lymphocytes malignancies (such as MCL) than to other subtypes of leukocytes.

In some embodiments, the targeting moiety targets a receptor selected from CD20, CCR7, CD3, CD4, CD5, CD8, CD16, CD19, CD20, CD21, CD22, CD25, CD28, CD35, CD40, CD45RA, CD45RO, CD52, CD62L, CD80, CD95, CD127, and CD137. In some embodiments, the targeting moiety targets a receptor selected from CD1, CD2, CD3, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, 0X40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7. In some embodiments, the targeting moiety targets a receptor selected from CD2, CD3, CD5 and CD7. In some embodiments, the targeting moiety targets a receptor selected from CD2, CD3, CD5, CD7, CD8, CD4, beta 7 integrin, beta 2 integrin, and C1q. In some embodiments, the targeting moiety targets CD117. In some embodiments, the targeting moiety targets CD90. In some embodiments, the targeting moiety targets a receptor selected from a mannose receptor, CD206 and C1q. In some embodiments, the targeting moiety is selected from T-cell receptor motif antibodies, T-cell α chain antibodies, T-cell β chain antibodies, T-cell γ chain antibodies, T-cell δ chain antibodies, CCR7 antibodies, CD3 antibodies, CD4 antibodies, CD5 antibodies, CD7 antibodies, CD8 antibodies, CD11b antibodies, CD11c antibodies, CD16 antibodies, CD19 antibodies, CD20 antibodies, CD21 antibodies, CD22 antibodies, CD25 antibodies, CD28 antibodies, CD34 antibodies, CD35 antibodies, CD40 antibodies, CD45RA antibodies, CD45RO antibodies, CD52 antibodies, CD56 antibodies, CD62L antibodies, CD68 antibodies, CD80 antibodies, CD95 antibodies, CD117 antibodies, CD127 antibodies, CD133 antibodies, CD137 (4-1BB) antibodies, CD163 antibodies, F4/80 antibodies, IL-4Rα antibodies, Sca-1 antibodies, CTLA-4 antibodies, GITR antibodies GARP antibodies, LAP antibodies, granzyme B antibodies, LFA-1 antibodies, transferrin receptor antibodies, and fragments thereof. In certain embodiments, the targeting moiety is any one described or contemplated in US20230312713A1, US20230203538A1, US20230320995A1, US20160145348, and US20110038941, each of which is incorporated by reference herein in its entirety.

In some embodiments, the targeting moiety is a small molecule. In some embodiments, the small molecule binds to an ectoenzyme on an immune cell, wherein the ectoenzyme is selected from the group consisting of CD38, CD73, adenosine 2a receptor, and adenosine 2b receptor. In some embodiments, the small molecule is mannose, a lectin, acivicin, biotin, or digoxigenin.

In some embodiments, the lipid nanoparticles may be targeted when conjugated/attached/associated with a targeting moiety such as an antibody, or a fragment thereof.

vi. Zwitterionic Amino Lipids

In some embodiments, an LNP comprises a zwitterionic lipid. In some embodiments, an LNP comprising a zwitterionic lipid does not comprise a phospholipid.

Zwitterionic amino lipids have been shown to be able to self-assemble into LNPs without phospholipids to load, stabilize, and release mRNAs intracellularly as described in U.S. patent application No. 20210121411, which is incorporated herein by reference in its entirety. Zwitterionic, ionizable cationic and permanently cationic helper lipids enable tissue-selective mRNA delivery and CRISPR-Cas9 gene editing in spleen, liver and lungs as described in Liu et al., Membrane-destablizing ionizable phospholipids for organ-selective mRNA delivery and CRISPR-Cas gene editing, Nat Mater. (2021), which is incorporated herein by reference in its entirety.

The zwitterionic lipids may have head groups containing a cationic amine and an anionic carboxylate as described in Walsh et al., Synthesis, Characterization and Evaluation of Ionizable Lysine-Based Lipids for siRNA Delivery, Bioconjug Chem. (2013), which is incorporated herein by reference in its entirety. Ionizable lysine-based lipids containing a lysine head group linked to a long-chain dialkylamine through an amide linkage at the lysine α-amine may reduce immunogenicity as described in Walsh et al., Synthesis, Characterization and Evaluation of Ionizable Lysine-Based Lipids for siRNA Delivery, Bioconjug Chem. (2013).

viii. Additional Lipid Components

In some embodiments, the LNP compositions of the present disclosure further comprise one or more additional lipid components capable of influencing the tropism of the LNP. In some embodiments, the LNP further comprises at least one lipid selected from DDAB, EPC, 14PA, 18BMP, DODAP, DOTAP, and C12-200 (see Cheng, et al. Nat Nanotechnol. 2020 April; 15(4): 313-320; Dillard, et al. PNAS 2021 Vol. 118 No. 52.).

In some embodiments, the LNP compositions of the present disclosure comprise, or further comprise one or more lipids selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4αMan), diolcoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Nα-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-α-histidinyl-Nα-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-malcimidomethyl)cyclohexane-carboxamide] (MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g. 1,2-dioleoyl-sn-glycero-3-phosphoethanolamine-N-[4-(p-maleimidophenyl) but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol (PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamine B-phosphatidylethanolamine lipid (Rh-PE), purified soy-derived mixture of phospholipids (SIOO), phosphatidylcholine (SM), 18-1-trans-PE, 1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy) methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.

### B. Exemplary LNP Compositions

In some embodiments, provided herein are LNPs comprising (a): at least one ionizable lipid; (b) at least one PEG lipid; (c) at least one structural lipid; and (d) at least one non-ionizable lipid and/or a zwitterionic lipid. In some embodiments, the LNPs further comprise an additional ionizable lipid, besides a compound disclosed herein. In some embodiments, the LNPs further comprise an additional lipid component, of any class, besides a compound disclosed herein.

In some embodiments, the PEG-lipid is selected from the group consisting of PEG-c-DOMG, PEG-DMG, PEG-DLPE, PEG-DMPE, PEG-DPPC, and PEG-DSPE.

In some embodiments, the structural lipid is selected from the group consisting of cholesterol, fecosterol, sitosterol, ergosterol, campesterol, stigmasterol, brassicasterol, tomatidine, ursolic acid, an alpha-tocopherol.

In some embodiments, the non-ionizable lipid is a phospholipid selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium(S)-2-ammonio-3-((((R)-2-(oleoyloxy)-3-(stearoyloxy)propoxy)oxidophosphoryl)oxy)propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(olcoyloxy)propyl)phosphate (18:1 DMP; LBPA), 1,2-diolcoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin.

In some embodiments, the non-ionizable lipid is a phospholipid selected from the group consisting of Egg Sphingomyelin (Egg SM/ESM/(2S,3R,E)-3-hydroxy-2-palmitamidooctadec-4-en-1-yl (2-(trimethylammonio)ethyl)phosphate), Brain or Porcine Sphingomyelin (Brain SM/(2S,3R,E)-3-hydroxy-2-stearamidooctadec-4-en-1-yl (2-(trimethylammonio)ethyl)phosphate), Milk or Bovine Sphingomyelin (Milk SM/(2S,3R,E)-3-hydroxy-2-tricosanamidooctadec-4-en-1-yl (2-(trimethylammonio)ethyl)phosphate), 28:0 SM (N-octacosanoyl-D-erythro-sphingosylphosphorylcholine), 14:0 SM (N-myristoyl-D-erythro-sphingosylphosphorylcholine), 16:1 SM (N-palmitoleoyl-D-erythro-sphingosylphosphorylcholine), 12:0 Dihydro SM (N-lauroyl-D-erythro-sphinganylphosphorylcholine), Lyso SM (Sphingosylphosphorylcholine), Lyso SM (Sphingosylphosphorylcholine), Lyso SM (dihydro) (Sphinganine Phosphorylcholine), 24:1 SM (N-nervonoyl-D-erythro-sphingosylphosphorylcholine), 24:0 SM (N-lignoceroyl-D-erythro-sphingosylphosphorylcholine), 18:1 SM (N-oleoyl-D-erythro-sphingosylphosphorylcholine), 18:0 SM (N-stearoyl-D-erythro-sphingosylphosphorylcholine), 17:0 SM (N-heptadecanoyl-D-erythro-sphingosylphosphorylcholine), 16:0 SM (N-palmitoyl-D-erythro-sphingosylphosphorylcholine), 12:0 SM (N-lauroyl-D-erythro-sphingosylphosphorylcholine), 06:0 SM (N-hexanoyl-D-erythro-sphingosylphosphorylcholine), 02:0 SM (N-acetyl-D-erythro-sphingosylphosphorylcholine), 3-O-methyl Lyso SM (3-O-methyl-spingosylphosphorylcholine), 3-O-methyl-N-methyl Lyso SM (3-O-methyl-N-methyl-spingosylphosphorylcholine), and 3-N-methyl Lyso SM (3-N-methyl-spingosylphosphorylcholine).

In some embodiments, (a) the PEG lipid is PEG2k-DMG or PEG2k-DSPE or a mixture thereof; (b) the structural lipid is cholesterol; and (c) the phospholipid, non-ionizable lipid or zwitterionic lipid is a sphingolipid or DSPC or a mixture thereof.

In some embodiments, the lipid component of the nanoparticle comprises: (a) about 0 mol % to about 10 mol % of PEG lipid; (b) about 0 mol % to about 30 mol % structural lipid; (c) about 20 mol % to about 45 mol % phospholipid, non-ionizable lipid or zwitterionic lipid; and (d) about 30 mol % to about 60 mol % of an ionizable lipid.

In some embodiments, the lipid component of the nanoparticle comprises: (a) about 1 mol % to about 2 mol % of PEG lipid; (b) about 25 mol % to about 40 mol % structural lipid; (c) about 20 mol % to about 45 mol % phospholipid, non-ionizable lipid or zwitterionic lipid; and (d) about 30 mol % to about 60 mol % of an ionizable lipid.

In some embodiments, the lipid component of the nanoparticle comprises: (a) about 2 mol % of PEG lipid; (b) about 25 mol % structural lipid; (c) about 40 mol % phospholipid, non-ionizable lipid or zwitterionic lipid; and (d) about 33 mol % of an ionizable lipid.

In some embodiments, the lipid component of the nanoparticle comprises: (a) about 2.5 mol % of PEG lipid; (b) about 39 mol % structural lipid; (c) about 10 mol % phospholipid, non-ionizable lipid or zwitterionic lipid; and (d) about 48.5 mol % of an ionizable lipid.

In some embodiments, the lipid component of the nanoparticle comprises: (a) about 1.5 mol % of PEG lipid; (b) about 40 mol % structural lipid; (c) about 10 mol % phospholipid, non-ionizable lipid or zwitterionic lipid; and (d) about 48.5 mol % of an ionizable lipid.

In some embodiments, the lipid component of the nanoparticle comprises: (a) about 1 mol % to about 3 mol % of PEG lipid; (b) about 15 mol % to about 35 mol % structural lipid; (c) about 30 mol % to about 60 mol % phospholipid, non-ionizable lipid or zwitterionic lipid; and (d) about 25 mol % to about 45 mol % of an ionizable lipid. In some embodiments, the lipid component of the nanoparticle comprises: (a) about 1 mol % to about 3 mol % of PEG lipid; (b) about 20 mol % to about 30 mol % structural lipid; (c) about 35 mol % to about 45 mol % phospholipid, non-ionizable lipid or zwitterionic lipid; and (d) about 28 mol % to about 40 mol % of an ionizable lipid. In some embodiments, the lipid component of the nanoparticle comprises: (a) about 1.5 mol % to about 2.5 mol % of PEG lipid; (b) about 20 mol % to about 30 mol % structural lipid; (c) about 35 mol % to about 45 mol % phospholipid, non-ionizable lipid or zwitterionic lipid; and (d) about 28 mol % to about 40 mol % of an ionizable lipid. In some embodiments, the lipid component of the nanoparticle comprises: (a) about 1 mol % to about 3 mol % of PEG lipid; (b) about 20 mol % to about 30 mol % structural lipid; (c) about 35 mol % to about 45 mol % phospholipid, non-ionizable lipid or zwitterionic lipid; and (d) about 28 mol % to about 40 mol % of an ionizable lipid; wherein the about 35 mol % to about 45 mol % phospholipid, non-ionizable lipid or zwitterionic lipid comprises two or more phospholipids, non-ionizable lipids or zwitterionic lipids. In some embodiments, the two or more phospholipids, non-ionizable lipids or zwitterionic lipids comprise at least one sphingolipid and at least one phosphatidycholine lipid. In some embodiments, the lipid component of the nanoparticle comprises: (a) about 1 mol % to about 3 mol % of PEG lipid; (b) about 20 mol % to about 30 mol % structural lipid; (c) about 35 mol % to about 45 mol % phospholipid; and (d) about 28 mol % to about 40 mol % of an ionizable lipid; wherein the about 35 mol % to about 45 mol % phospholipid comprises a mixture of phosphatidylcholine, phosphatidylserine, phosphoethanolamine, and sphingoid lipids. In some embodiments, the lipid component of the nanoparticle comprises: (a) about 1 mol % to about 3 mol % of PEG lipid; (b) about 20 mol % to about 30 mol % structural lipid; (c) about 35 mol % to about 45 mol % phospholipid; and (d) about 28 mol % to about 40 mol % of an ionizable lipid; wherein the about 35 mol % to about 45 mol % phospholipid comprises a mixture of phosphatidylcholine, phosphatidylserine, phosphoethanolamine, and sphingoid lipids, such that no single phospholipid makes up more than 25 mol % of the total lipid content of the nanoparticle. In some embodiments, the lipid component of the nanoparticle comprises: (a) about 1 mol % to about 3 mol % of PEG lipid; (b) about 20 mol % to about 30 mol % structural lipid; (c) about 35 mol % to about 45 mol % phospholipid; and (d) about 28 mol % to about 40 mol % of an ionizable lipid; wherein the about 35 mol % to about 45 mol % phospholipid comprises a mixture of phosphatidylcholine and sphingoid lipids, such that no single phospholipid makes up more than 25 mol % of the total lipid content of the nanoparticle. In some embodiments, the lipid component of the nanoparticle comprises: (a) about 1 mol % to about 3 mol % of PEG lipid; (b) about 15 mol % to about 35 mol % structural lipid; (c) about 30 mol % to about 60 mol % phospholipid; and (d) about 25 mol % to about 45 mol % of an ionizable lipid; wherein the about 30 mol % to about 60 mol % phospholipid comprises a mixture of phosphatidylcholine and sphingoid lipids, such that no single phospholipid makes up more than 30 mol % of the total lipid content of the nanoparticle. In some embodiments, the lipid component of the nanoparticle comprises: (a) about 1 mol % to about 3 mol % of PEG2k-DMG; (b) about 15 mol % to about 35 mol % cholesterol; (c) about 30 mol % to about 60 mol % phospholipid; and (d) about 25 mol % to about 45 mol % of an ionizable lipid; wherein the about 30 mol % to about 60 mol % phospholipid comprises a mixture of phosphatidylcholine and sphingoid lipids, such that no single phospholipid makes up more than 30 mol % of the total lipid content of the nanoparticle. In some embodiments, the lipid component of the nanoparticle comprises: (a) about 1 mol % to about 3 mol % of PEG2k-DMG; (b) about 15 mol % to about 35 mol % cholesterol; (c) about 30 mol % to about 60 mol % phospholipid; and (d) about 25 mol % to about 45 mol % of an ionizable lipid; wherein the about 30 mol % to about 60 mol % phospholipid comprises a mixture of DSPC and sphingomyelin, such that the mol % of DPSC is not greater than 30 mol % of the total lipid content and the mol % of sphingomyelin is not greater than 30 mol % of the total lipid content. In some embodiments, the lipid component of the nanoparticle comprises: (a) about 1 mol % to about 3 mol % of PEG2k-DMG; (b) about 20 mol % to about 30 mol % cholesterol; (c) about 35 mol % to about 45 mol % phospholipid; and (d) about 28 mol % to about 40 mol % of an ionizable lipid; wherein the about 35 mol % to about 45 mol % phospholipid comprises a mixture of DSPC and sphingomyelin, such that the mol % of DPSC is not greater than 25 mol % of the total lipid content and the mol % of sphingomyelin is not greater than 25 mol % of the total lipid content.

In some embodiments, the lipid component of the nanoparticle comprises: (a) about 2 mol % PEG lipid; (b) about 25 mol % structural lipid; (c) about 40 mol % phospholipid; and (d) about 33 mol % of an ionizable lipid. In some embodiments, the lipid component of the nanoparticle comprises: (a) about 1 mol % PEG lipid; (b) about 26 mol % structural lipid; (c) about 40 mol % phospholipid; and (d) about 33 mol % of an ionizable lipid. In some embodiments, the lipid component of the nanoparticle comprises: (a) about 1.5 mol % PEG lipid; (b) about 25.5 mol % structural lipid; (c) about 40 mol % phospholipid; and (d) about 33 mol % of an ionizable lipid. In some embodiments, the lipid component of the nanoparticle comprises: (a) about 2 mol % PEG lipid; (b) about 20 mol % structural lipid; (c) about 35 mol % phospholipid; and (d) about 43 mol % of an ionizable lipid. In some embodiments, the lipid component of the nanoparticle comprises: (a) about 2 mol % PEG lipid; (b) about 15 mol % structural lipid; (c) about 34.5 mol % phospholipid; and (d) about 48.5 mol % of an ionizable lipid. In some embodiments, the lipid component of the nanoparticle comprises: (a) about 2 mol % PEG lipid; (b) about 10 mol % structural lipid; (c) about 39.5 mol % phospholipid; and (d) about 48.5 mol % of an ionizable lipid. In some embodiments, the lipid component of the nanoparticle comprises: (a) about 2 mol % PEG lipid; (b) about 30 mol % structural lipid; (c) about 35 mol % phospholipid; and (d) about 33 mol % of an ionizable lipid. In some embodiments, the lipid component of the nanoparticle comprises: (a) about 2.3 mol % PEG lipid; (b) about 25 mol % structural lipid; (c) about 38.2 mol % phospholipid; and (d) about 34.5 mol % of an ionizable lipid. In some embodiments, the lipid nanoparticle is any one of the aforementioned embodiments in this paragraph, wherein the PEG lipid is PEG2k-DMG. In some embodiments, the lipid nanoparticle is any one of the aforementioned embodiments in this paragraph, wherein the structural lipid is cholesterol. In some embodiments, the lipid nanoparticle is any one of the aforementioned embodiments in this paragraph, wherein the phospholipid content comprises a phosphatidylcholine lipid, a sphingoid lipid or combinations thereof.

In some embodiments, the payloads are encapsulated in nanoparticles (e.g., LNPs) for delivery. In embodiments, a nanoparticle can comprise an ionizable lipid, a phospholipid, a PEG lipid, and a structural lipid. In certain embodiments, the lipid component of the nanoparticle composition comprises about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In certain embodiments, the lipid component of the nanoparticle composition comprises about 20 mol % to about 45 mol % ionizable lipid, about 30 mol % to about 60 mol % phospholipid, about 10 mol % to about 30 mol % structural lipid, and about 0 mol % to about 10 mol % of PEG lipid, provided that the total mol % does not exceed 100%. In some embodiments, the lipid component of the nanoparticle composition comprises about 35 mol % to about 55 mol % ionizable lipid, about 5 mol % to about 25 mol % phospholipid, about 30 mol % to about 40 mol % structural lipid, and about 0 mol % to about 10 mol % of PEG lipid, provided that the total mol % does not exceed 100%. In some embodiments, the lipid component of the nanoparticle composition comprises about 30 mol % to about 40 mol % ionizable lipid, about 35 mol % to about 45 mol % phospholipid, about 20 mol % to about 30 mol % structural lipid, and about 0.5 mol % to about 5 mol % of PEG lipid, provided that the total mol % does not exceed 100%. In a particular embodiment, the lipid component comprises about 50 mol % ionizable lipid, about 10 mol % phospholipid, about 38.5 mol % structural lipid, and about 1.5 mol % of PEG lipid. In another particular embodiment, the lipid component comprises about 40 mol % ionizable lipid, about 20 mol % phospholipid, about 38.5 mol % structural lipid, and about 1.5 mol % of PEG lipid. In another particular embodiment, the lipid component comprises about 48.5 mol % ionizable lipid, about 10 mol % phospholipid, about 40 mol % structural lipid, and about 1.5 mol % of PEG lipid. In another particular embodiment, the lipid component comprises about 48.5 mol % ionizable lipid, about 10 mol % phospholipid, about 39 mol % structural lipid, and about 2.5 mol % of PEG lipid. In another particular embodiment, the lipid component comprises about 48.5 mol % ionizable lipid, about 10 mol % phospholipid, about 38.5 mol % structural lipid, and about 3 mol % of PEG lipid. In another particular embodiment, the lipid component comprises about 48.5 mol % ionizable lipid, about 10 mol % phospholipid, about 38 mol % structural lipid, and about 3.5 mol % of PEG lipid. In another particular embodiment, the lipid component comprises about 33 mol % ionizable lipid, about 40 mol % phospholipid, about 25 mol % structural lipid, and about 2 mol % of PEG lipid. In some embodiments, the phospholipid is DOPE or DSPC. In some embodiments, the phospholipid is DSPC. In some embodiments, the phospholipid is a sphingolipid. In some embodiments, the phospholipid is a sphingomyelin. In other embodiments, the PEG lipid is PEG-DMG (eg. PEG2K-DMG). In other embodiments, the PEG lipid is PEG-DSPE (eg. PEG2K-DSPE). In other embodiments, the PEG lipid is PEG-DMPE (eg. PEG2K-DMPE). In other embodiments, the PEG lipid is PEG-DPPE (eg. PEG2K-DPPE). In other embodiments, the structural lipid is cholesterol. In other embodiments, the PEG lipid is PEG-DMG and/or the structural lipid is cholesterol. In some embodiments, the PEG lipids is PEG2K-DMG, the structural lipid is cholesterol, and the phospholipid is DSPC. In some embodiments, the PEG lipids is PEG2K-DMG, the structural lipid is cholesterol, and the phospholipid is sphingomyelin.

In some embodiments, the PEG lipids is PEG-DMG, the structural lipid is cholesterol, and the phospholipid is a mixture of DSPC and sphingomyelin. In certain embodiments, the LNP comprises about 33 mol % ionizable lipid (eg. at least one ionizable lipid of a Formula described herein), about 40 mol % of a sphingolipid, about 25 mol % cholesterol and about 2 mol % PEG2K-DMG. In some embodiments, the PEG lipids is PEG2K-DSPE, the structural lipid is cholesterol, and the phospholipid is DSPC. In some embodiments, the PEG lipids is PEG2K-DSPE, the structural lipid is cholesterol, and the phospholipid is sphingomyelin. In some embodiments, the PEG lipids is PEG-DSPE, the structural lipid is cholesterol, and the phospholipid is a mixture of DSPC and sphingomyelin. In some embodiments, the PEG lipids is PEG2K-DMG, the structural lipid is cholesterol, and the phospholipid is DOPE. In some embodiments, the PEG lipids is PEG2K-DMG, the structural lipid is cholesterol, and the phospholipid is DOPC. In some embodiments, the PEG lipids is PEG2K-DMG, the structural lipid is cholesterol, and the phospholipid is DLPC. In some embodiments, the PEG lipids is PEG2K-DMG, the structural lipid is cholesterol, and the phospholipid is DOPS. In some embodiments, the PEG lipids is PEG-DMG, the structural lipid is cholesterol, and the phospholipid is a mixture of a phosphatidylcholine lipid and a sphingolipid. In some embodiments, the PEG lipids is PEG-DMG, the structural lipid is cholesterol, and the phospholipid is a mixture of a phosphatidylcholine lipid and phosphatidylserine lipid. In some embodiments, the PEG lipids is PEG-DMG, the structural lipid is cholesterol, and the phospholipid is a mixture of a phosphatidylcholine lipid and a phosphoethanolamine lipid. In some embodiments, the PEG lipids is PEG-DMG, the structural lipid is cholesterol, and the phospholipid is a mixture of a sphingolipid and phosphatidylserine lipid. In some embodiments, the PEG lipids is PEG-DMG, the structural lipid is cholesterol, and the phospholipid is a mixture of a sphingolipid and a phosphoethanolamine lipid. In certain embodiments, the LNP comprises about 33 mol % ionizable lipid, about 20 mol % of a sphingolipid, about 20 mol % of a non-sphingolipid phospholipid, about 25 mol % cholesterol and about 2 mol % of a PEGylated lipid. In certain embodiments, the LNP comprises about 33 mol % ionizable lipid, about 10 mol % of a sphingolipid, about 30 mol % of a non-sphingolipid phospholipid, about 25 mol % cholesterol and about 2 mol % of a PEGylated lipid. In certain embodiments, the LNP comprises about 33 mol % ionizable lipid, about 30 mol % of a sphingolipid, about 10 mol % of a non-sphingolipid phospholipid, about 25 mol % cholesterol and about 2 mol % of a PEGylated lipid. In certain embodiments, the LNP comprises about 33 mol % ionizable lipid, about 20 mol % sphingomyelin, about 20 mol % of a DSPC, about 25 mol % cholesterol and about 2 mol % of a PEGylated lipid. In certain embodiments, the LNP comprises about 33 mol % ionizable lipid, about 10 mol % sphingomyelin, about 30 mol % of a DSPC, about 25 mol % cholesterol and about 2 mol % of a PEGylated lipid. In certain embodiments, the LNP comprises about 33 mol % ionizable lipid, about 30 mol % sphingomyelin, about 10 mol % of a DSPC, about 25 mol % cholesterol and about 2 mol % of a PEGylated lipid. In certain embodiments, the LNP comprises about 33 mol % ionizable lipid, about 25 mol % cholesterol, about 2 mol % of a PEGylated lipid, and about 40% of a mixture of phosphatidylcholine, phosphatidylserine, phosphoethanolamine, and sphingoid lipids. In certain embodiments, the LNP comprises about 33 mol % ionizable lipid, about 25 mol % cholesterol, about 2 mol % of a PEGylated lipid, and about 40% of a mixture of phosphatidylcholine, phosphatidylserine, phosphoethanolamine, and sphingoid lipids, wherein each of the phosphatidylcholine, phosphatidylserine, phosphoethanolamine, and sphingoid lipids is present in an amount less than 30 mol % of the total lipid component of the LNP. In certain embodiments, the LNP comprises about 33 mol % ionizable lipid, about 25 mol % cholesterol, about 2 mol % of a PEGylated lipid, and about 40% of a mixture of phosphatidylcholine, phosphatidylserine, phosphoethanolamine, and sphingoid lipids, wherein each of the phosphatidylcholine, phosphatidylserine, phosphoethanolamine, and sphingoid lipids is present in an amount less than 25 mol % of the total lipid component of the LNP. In certain embodiments, LNP is any one of the aforementioned in this paragraph wherein the PEG lipid is PEG2k-DMG. In certain embodiments, LNP is any one of the aforementioned in this paragraph wherein the PEG lipid is PEG2k-DSPE.

In another particular embodiment, LNP comprises about 33 mol % ionizable lipid, about 40 mol % DSPC, about 25 mol % cholesterol, and about 2 mol % of PEG lipid. In another particular embodiment, LNP comprises about 33 mol % ionizable lipid, about 40 mol % sphingomyelin, about 25 mol % cholesterol, and about 2 mol % of PEG lipid. In another particular embodiment, LNP comprises about 33 mol % ionizable lipid, about 40 mol % DOPE, about 25 mol % cholesterol, and about 2 mol % of PEG lipid. In another particular embodiment, LNP comprises about 33 mol % ionizable lipid, about 40 mol % DOPC, about 25 mol % cholesterol, and about 2 mol % of PEG lipid. In another particular embodiment, LNP comprises about 33 mol % ionizable lipid, about 40 mol % DLPC, about 25 mol % cholesterol, and about 2 mol % of PEG lipid. In another particular embodiment, LNP comprises about 33 mol % ionizable lipid, about 40 mol % DOPS, about 25 mol % cholesterol, and about 2 mol % of PEG lipid. In another particular embodiment, LNP comprises about 33 mol % ionizable lipid, about 40 mol % phospholipid, about 25 mol % cholesterol, and about 2 mol % of PEG lipid. In another particular embodiment, LNP comprises about 33 mol % ionizable lipid, about 20 mol % sphingomyelin, about 20 mol % DSPC, about 25 mol % cholesterol, and about 2 mol % of PEG lipid. In certain embodiments, LNP is any one of the aforementioned in this paragraph wherein the PEG lipid is PEG2k-DMG. In certain embodiments, LNP is any one of the aforementioned in this paragraph wherein the PEG lipid is PEG2k-DSPE.

In certain embodiments, the LNP comprises about 43 mol % ionizable lipid, about 15 mol % of a sphingolipid, about 15 mol % of a non-sphingolipid phospholipid, about 25 mol % cholesterol and about 2 mol % of a PEGylated lipid. In certain embodiments, the LNP comprises about 33 mol % ionizable lipid, about 25 mol % of a sphingolipid, about 15 mol % of a non-sphingolipid phospholipid, about 25 mol % cholesterol and about 2 mol % of a PEGylated lipid. In certain embodiments, the LNP comprises about 33 mol % ionizable lipid, about 15 mol % of a sphingolipid, about 25 mol % of a non-sphingolipid phospholipid, about 25 mol % cholesterol and about 2 mol % of a PEGylated lipid. In some embodiments, the PEG lipid is PEG2K-DSPE, the structural lipid is cholesterol, and the phospholipid is a mixture of DSPC and sphingomyelin. In some embodiments, the PEG lipid is PEG2K-DMG, the structural lipid is cholesterol, and the phospholipid is a mixture of DSPC and sphingomyelin. In certain embodiments, the LNP comprises about 48.5 mol % ionizable lipid, about 10 mol % of a phospholipid (such as DSPC), about 40 mol % cholesterol and about 1.5 mol % PEG2K-DSPE. In certain embodiments, the LNP comprises about 48.5 mol % ionizable lipid, about 10 mol % of a phospholipid (such as DSPC), about 40 mol % cholesterol and about 1.5 mol % PEG2K-DMG. In certain embodiments, the LNP comprises about 48.5 mol % ionizable lipid, about 10 mol % of a phospholipid (such as DSPC), about 39 mol % cholesterol and about 2.5 mol % PEG2K-DSPE.

In some embodiments, the PEG lipid is PEG2K-DPPE, the structural lipid is cholesterol, and the phospholipid is a DSPC or a mixture of DSPC and sphingomyelin. In some embodiments, the PEG lipid is PEG2K-DPPE, the structural lipid is cholesterol, and the phospholipid is a mixture of DSPC and sphingomyelin. In certain embodiments, the LNP comprises about 48.5 mol % ionizable lipid, about 10 mol % of a phospholipid (such as DSPC), about 40 mol % cholesterol and about 1.5 mol % PEG2K-DPPE. In certain embodiments, the LNP comprises about 48.5 mol % ionizable lipid, about 10 mol % of a phospholipid (such as DSPC), about 39.5 mol % cholesterol and about 2 mol % PEG2K-DPPE. In certain embodiments, the LNP comprises about 48.5 mol % ionizable lipid, about 10 mol % of a phospholipid (such as DSPC), about 39 mol % cholesterol and about 2.5 mol % PEG2K-DPPE. In certain embodiments, the LNP comprises about 48.5 mol % ionizable lipid, about 10 mol % of a phospholipid (such as DSPC), about 38.5 mol % cholesterol and about 3 mol % PEG2K-DPPE. In certain embodiments, the LNP comprises about 48.5 mol % ionizable lipid, about 10 mol % of a phospholipid (such as DSPC), about 38 mol % cholesterol and about 3.5 mol % PEG2K-DPPE.

In some embodiments, the mRNA payloads are encapsulated in nanoparticles (e.g., LNPs) for delivery. In embodiments, a nanoparticle can comprise an ionizable lipid, a phospholipid, a PEG lipid, and a structural lipid. In certain embodiments, the lipid component of the nanoparticle composition comprises about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition comprises about 35 mol % to about 55 mol % ionizable lipid, about 5 mol % to about 25 mol % phospholipid, about 30 mol % to about 40 mol % structural lipid, and about 0 mol % to about 10 mol % of PEG lipid. In a particular embodiment, the lipid component comprises about 50 mol % ionizable lipid, about 10 mol % phospholipid, about 38.5 mol % structural lipid, and about 1.5 mol % of PEG lipid. In another particular embodiment, the lipid component comprises about 40 mol % ionizable lipid, about 20 mol % phospholipid, about 38.5 mol % structural lipid, and about 1.5 mol % of PEG lipid. In another particular embodiment, the lipid component comprises about 48.5 mol % ionizable lipid, about 10 mol % phospholipid, about 40 mol % structural lipid, and about 1.5 mol % of PEG lipid. In another particular embodiment, the lipid component comprises about 48.5 mol % ionizable lipid, about 10 mol % phospholipid, about 39 mol % structural lipid, and about 2.5 mol % of PEG lipid. In some embodiments, the phospholipid is DOPE or DSPC. In other embodiments, the PEG lipid is PEG-DMG (eg. PEG2K-DMG). In other embodiments, the PEG lipid is PEG-DSPE (eg. PEG2K-DSPE). In other embodiments, the PEG lipid is PEG-DMPE (eg. PEG2K-DMPE). In other embodiments, the structural lipid is cholesterol. In other embodiments, the PEG lipid is PEG-DMG and/or the structural lipid is cholesterol.

In some embodiments, the LNP further comprises a targeting moiety. In some embodiments, the targeting moiety is an antibody or a fragment thereof.

The characteristics of a nanoparticle composition may depend on the components thereof. For example, a nanoparticle composition including cholesterol as a structural lipid may have different characteristics than a nanoparticle composition that comprises a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

In some embodiments, the mean size of a nanoparticle composition is between 10 s of nm and 100 s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition is from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition is from about 70 nm to about 100 nm. In a particular embodiment, the mean size is about 80 nm. In other embodiments, the mean size is about 100 nm.

In some embodiments, the LNPs of the present disclosure can be characterized by their shape. In some embodiments, the LNPs are essentially spherical. In some embodiments, the LNPs are essentially rod-shaped (i.e., cylindrical). In some embodiments, the LNPs are essentially disk shaped.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

The Zeta potential of a nanoparticle composition may be used to indicate the electrokinetic potential of the composition. For example, the Zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the Zeta potential of a nanoparticle composition is from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV, to about +20 mV, from about 0 mV to about +15 mV, from about 0 mV to about +10 mV, from about 0 mV to about +5 mV, from about +5 mV to about +20 mV, from about +5 mV, to about +15 mV, or from about +5 mV to about +10 mV.

The efficiency of encapsulation of a payload describes the amount of payload that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of payload in a solution containing the nanoparticle composition before and after breaking up the nanoparticle composition with one or more organic solvents or detergents. Fluorescence may be used to measure the amount of free payload in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency is at least 80%. In certain embodiments, the encapsulation efficiency is at least 90%.

The amount of active agent in a nanoparticle composition may depend on the size, composition, desired target and/or application, or other properties of the nanoparticle composition as well as on the properties of the active agent. For example, the amount of active agent useful in a nanoparticle composition may depend on the size, sequence, and other characteristics of the active agent. The relative amounts of active agent and other elements (e.g., lipids) in a nanoparticle composition may also vary. In some embodiments, the wt/wt ratio of the lipid component to payload in a nanoparticle composition is from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a payload in a nanoparticle composition may, for example, be measured using absorption spectroscopy (e.g., ultraviolet-visible spectroscopy).

In some embodiments, a nanoparticle composition of the present disclosure is formulated to provide a specific N:P ratio. The N:P ratio of the composition refers to the molar ratio of nitrogen atoms in one or more lipids to the number of phosphate groups in an RNA active agent (e.g., a linear or circular mRNA payload). In general, a lower N:P ratio is preferred. The one or more enzymes, lipids, and amounts thereof is selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio is from about 2:1 to about 8:1. In other embodiments, the N:P ratio is from about 5:1 to about 8:1. For example, the N:P ratio is about 5.0:1, about 5.5:1, about 5.67:1, about 6.0:1, about 6.5:1, or about 7.0:1.

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

Lipids and their method of preparation are disclosed in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548 and Semple et. al, Nature Biotechnology, 2010, 28, 172-176, the full disclosures of which are herein incorporated by reference in their entirety for all purposes.

A nanoparticle composition may comprise any substance useful in pharmaceutical compositions. For example, the nanoparticle composition may comprise one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be comprised. Pharmaceutically acceptable excipients are well known in the art (see for example Remington's The Science and Practice of Pharmacy, 21st Edition, A. R. Gennaro: Lippincott, Williams & Wilkins, Baltimore, Md., 2006).

### III. LNP Payload

In various aspects, the LNPs described herein may be used to deliver a payload of interest to a biological target, e.g., to a cell or a bodily tissue. The term “payload” refers to an active substance, such as a small molecule, polypeptide, peptide, carbohydrate, or nucleic acid molecule, and includes, without limitation, mRNA molecules (including linear and circular mRNA) and non-coding RNAs (e.g., guide RNAs) which are encapsulated within the LNPs described herein. In various embodiments, the payload is an RNA molecule, which may be linear or circular and may comprise one or more functional nucleotide sequences of interest, which may include, but are not limited to coding and non-coding nucleotide sequences. In various embodiments, the non-coding nucleotide sequences may comprise regulatory elements that influence RNA post-transcriptional processing, nuclear translation control sequences, and sequences which encode one or more biological products of interest, e.g., a therapeutic protein or nucleobase editing system, among other sequence elements that may impact the functioning of the RNA or its encoded products. As used herein, the term “coding region of interest” or “product coding region” or the like may be used to refer to the encoded one or more biological products of interest. Equivalently, a product coding region may be referred to as a “product expression sequence.”

### A. Gene Editing Systems and Components for Treatment of Hemoglobinopathies

In certain embodiments, the present disclosure provides compositions and methods of delivering gene editing tools. Genome editing tools encompass a diverse set of technologies that can make many types of genomic alterations in various contexts. These technologies have evolved over the last couple of decades to provide a range of user-programmable editing tools that include ZFN (zinc finger) nuclease editing systems, meganuclease editing systems, and TALENS (transcription activator-like effector nucleases). The past decade has seen an explosive growth in a new generation of genome editing systems based on components from bacterial immune pathways, including CRISPR (clustered regularly interspaced short palindromic repeats) and the associated CRISPR-associated proteins (e.g., CRISPR-Cas9) (Jinek et al., “A programmable dual-RNA-guided DNA endonuclease in adaptive bacterial immunity,” Science, Vol. 337 (6096), pp. 816-821), meganuclease editors (Boissel et al., “megaTALs: a rare-cleaving nuclease architecture for therapeutic genome engineering,” Nucleic Acids Research 42: pp. 2591-2601) and bacterial retron systems (Schubert et al., “High-throughput functional variant screens via in vivo production of single-stranded DNA,” PNAS, Apr. 27, 2021, Vol. 118(18), pp. 1-10). In particular, CRISPR-Cas9 has been derivatized in numerous ways to expand upon its guide RNA-based programmable double-strand cutting activity to form systems ranging from finding alternative CRISPR Cas nuclease enzymes having different PAM requirements and cutting properties (e.g., Cas12a, Cas12f, Cas13a, and Cas13b) to base editing (Komor et al., “Programmable editing of a target base in genomic DNA without double-stranded DNA cleavage,” Nature, May 19, 2016, 533 (7603); pp. 420-424 [cytosine base editors or CBEs] and Gaudelli et al., “Programmable base editing of A-T to G-C in genomic DNA without DNA cleavage,” Nature, Vol. 551, pp. 464-471 [adenine base editors or ABEs]) to prime editing (Anzalone et al., “Search-and-replace genome editing without double-strand breaks or donor DNA,” Nature, December 2019, 576 (7789): pp. 149-157) to twin prime editing (Anzalone et al., “Programmable deletion, replacement, integration and inversion of large DNA sequences with twin prime editing,” Nature Biotechnology, Dec. 9, 2021, vol. 40, pp. 731-740) to epigenetic editing (Kungulovski and Jeltsch, “Epigenome Editing: State of the Art, Concepts, and Perspective,” Trends in Genetics, Vol. 32, 206, pp. 101-113) to CRISPR-directed integrase editing (Yarnell et al., “Drag-and-drop genome insertion of large sequences without double-stranded DNA cleavage using CRISPR-directed integrases,” Nature Biotechnology, Nov. 24, 2022, (“PASTE”)), among others. Such editing systems may be implemented for treating diseases, such as hemoglobinopathies.

While the expansion of genome editing tools has exploded, the development of safe and effective gene editing tool delivery systems has lagged behind. There remain numerous challenges associated with the delivery of gene editing tools—including, but not limited to, CRISPR-Cas9 and alternative Cas nuclease editors, retron editors, base editors, prime editors, twin prime editors, epigenetic editors, and integrase editors—to achieve safe and effective therapeutic application of such tools in cells and patients for treating disease and/or otherwise modifying the nucleotide sequence of a target nucleic acid molecule (e.g., a gene or genome). That said, the use of lipid nanoparticles (LNPs) has emerged as a leading delivery option for the safe, effective, and targeted delivery of gene editing tools to target tissues and cells. However, there remains a need for improved LNPs, including better performing ionizable lipids, that will enhance the targeted delivery of LNP-based gene editing tools. Preferably, such improved LNPs would protect payloads from degradation and clearance while achieving targeted delivery, be suitable for systemic or local delivery, and provide delivery of a wide variety of gene editing tools, such as those mentioned above. In addition, such improved LNP-based therapeutics should exhibit low toxicity and provide an adequate therapeutic index, such that patient treatment at an effective dose of the LNP minimizes risk to the patient while maximizing therapeutic benefit. The present disclosure provides these and related advantages.

In one embodiment, the present disclosure provides pharmaceutical compositions comprising lipid nanoparticles comprising one or more gene editing systems, or components thereof, capable of editing, modifying, or altering a polynucleotide sequence that results in treatment of a hemoglobinopathy.

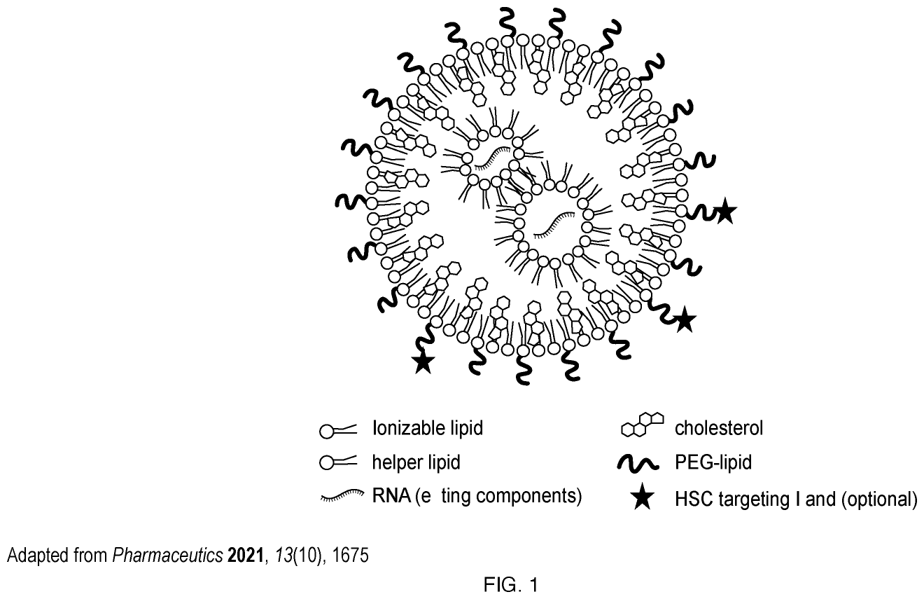

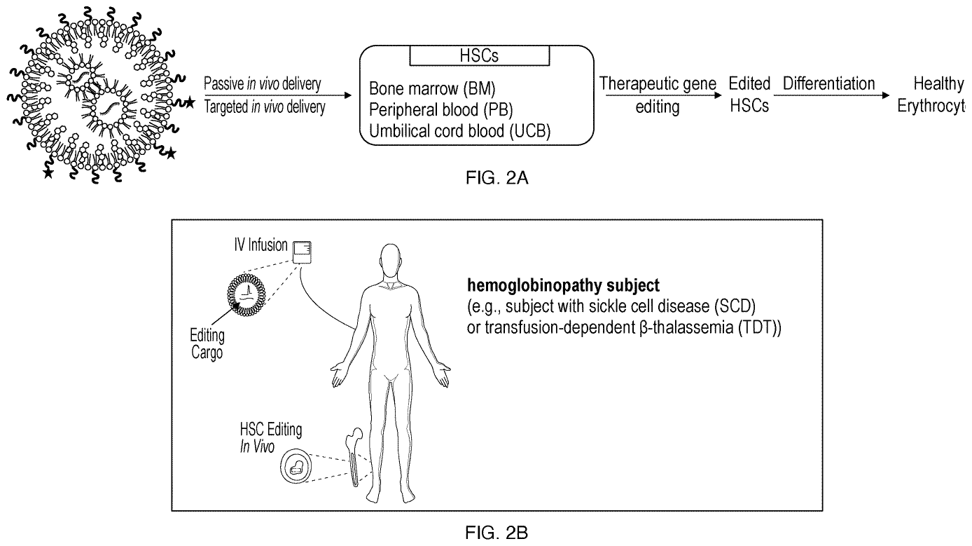

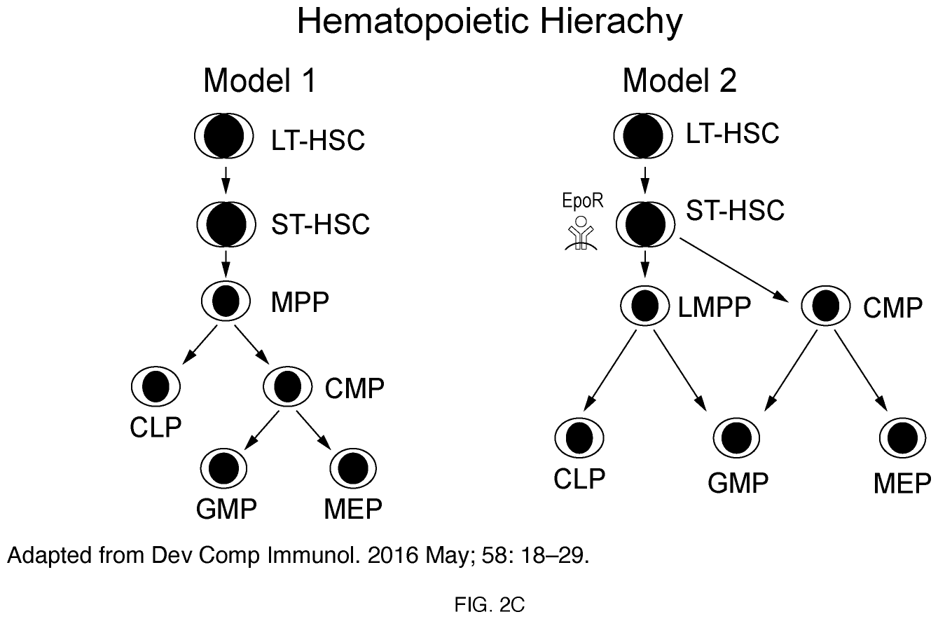

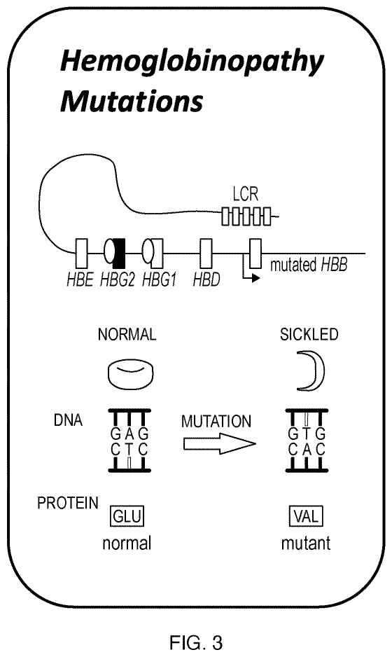

In certain embodiments, the gene editing system is a system that corrects the sickle cell mutation by repairing the β-globin gene. In general, strategies of repairing the β-globin gene in order to address a β-hemoglobinopathy are illustrated in the central panel of FIG. 1. In certain embodiments, the gene editing system corrects the sickle cell mutation through homology directed repair. In certain embodiments, the gene editing system corrects the sickle cell mutation through non-homologous end-joining (NHEJ), by targeting aberrant splicing sites in introns I and II, resulting in restoration of functional β-globin transcripts. In certain embodiments, the gene editing system corrects the sickle cell mutation through a single base pair gene correction. In certain embodiments, the gene editing system is a prime editing or base editing system capable of executing a single base pair edit. In certain embodiments, the gene editing system executes a single base pair correction, converting the aberrant thymine to the normal adenine, thereby converting the aberrant valine amino acid in the mutant hemoglobin to the normal glutamic acid. In certain embodiments, the gene editing system executes a single base pair correction, converting the aberrant thymine to cytosine, thereby converting the aberrant valine amino acid in the mutant hemoglobin to alanine, which results in a functional, nonpathogenic hemoglobin variant (HbG Makassar).

In certain embodiments, the gene editing system is a system that reactivates γ-globin production. In general, strategies of reactivating γ-globin production in order to address a β-hemoglobinopathy are illustrated in the right panel of FIG. 1. One method of treating SCD and beta-Thalassemia is to reactivate production of functional fetal hemoglobin (γ-globin) to compensate for the pathogenic mutant β-globin. In certain embodiments, the gene editing system reactivates γ-globin production by deleting inhibitory sequences, or portions thereof. In certain embodiments, the gene editing system eliminates an HbF inhibitory sequence, or a portion thereof, thereby reactivating γ-globin production. In certain embodiments, the gene editing system juxtaposes the γ-globin promoters to remote enhancer regions. In certain embodiments, the gene editing system targets the HBG1 and/or HBG2 promoters, resulting in disruption of binding sites for γ-globin transcriptional repressors, leading to HbF expression. In certain embodiments, the gene editing system comprises base editors that reproduce HPFH mutations that create binding sites for transcriptional activators (such as KLF1) or disrupt repressor binding sites (such as BCL11A). In certain embodiments, the gene editing system disrupts BCL11A binding sites. In certain embodiments, the gene editing system disrupts the GATA1 motif in the erythroid-specific intronic enhancer of BCL11A. Disruption of BCL11A can be achieved using nucleases or base editors, and BCL11A knock-down results in reactivation of γ-globin expression.

In certain embodiments, an LNP of the present disclosure comprises a gene editing system, or a polynucleotide encoding the same, disclosed in any one of the documents cited in Table X1. In certain embodiments, an LNP of the present disclosure comprises a nuclease, or a polynucleotide encoding the same, disclosed in any one of the documents cited in Table X1. In certain embodiments, an LNP of the present disclosure comprises a Cas9 or ortholog thereof, or a polynucleotide encoding the same, disclosed in any one of the documents cited in Table X1. In certain embodiments, an LNP of the present disclosure comprises a Cas12a (formerly known as Cpf1), or Type V protein, or a polynucleotide encoding the same, disclosed in any one of the documents cited in Table X1. In certain embodiments, an LNP of the present disclosure comprises a Cas12a, Type V or Cas9 ortholog protein, or a polynucleotide encoding the same, disclosed in any one of the documents cited in Table X1. In certain embodiments, an LNP of the present disclosure comprises a nickase, or a polynucleotide encoding the same, disclosed in any one of the documents cited in Table X1. In certain embodiments, an LNP of the present disclosure comprises a zinc finger protein, or a polynucleotide encoding the same, disclosed in any one of the documents cited in Table X1. In certain embodiments, an LNP of the present disclosure comprises a TALEN, or a polynucleotide encoding the same, disclosed in any one of the documents cited in Table X1. In certain embodiments, an LNP of the present disclosure comprises a guide RNA, or a portion or segment thereof, disclosed in any one of the documents cited in Table X1. In certain embodiments, an LNP of the present disclosure comprises a spacer sequence disclosed in any one of the documents cited in Table X1.

TABLE X1
Patent and Patent Application References
US Patents

| | | | | | |
|---|---|---|---|---|---|
| 11,242,525 | 11,572,543 | 11,268,077 | 11,466,271 | 9,957,501 | 10,808,020 |
| 11,021,696 | 10,563,184 | 11,155,803 | 10,738,305 | 11,344,609 | 10,435,677 |
| 11,466,271 | | | | | |

US Patent Application Publications

| | | | |
|---|---|---|---|
| US20200339979A1 | US20210180091A1 | US20200140896A1 | US20190010495A1 |
| US20210254061A1 | US20210040506A1 | US20230002760A1 | US20230018543A1 |
| US20180273609A1 | US20220047637A1 | US20190184035A1 | US20200255857A1 |
| US20200399626A1 | US20220228142A1 | US20180200387A1 | US20180030438A1 |
| US20220401530A1 | US20210371858A1 | US20190010495A1 | US20210180091A1 |
| US20210008161A1 | US20210155927A1 | US20200330609A1 | US20200384033A1 |

PCT Publications

| | | | |
|---|---|---|---|
| WO2022240787A1 | WO2021119040A1 | WO2023014839A1 | WO2023039440A2 |
| WO2022120094A2 | WO2022155458A1 | WO2023019227A1 | WO2023019226A1 |
| WO2022067122A1 | WO2023019225A2 | WO2021163587A1 | WO2024020346A1 |
| WO2017191503A1 | WO2021144692A1 | | |

Each reference of Table X1 is incorporated by reference herein in its entirety.

In certain embodiments, an LNP of the present disclosure comprises a nuclease, or a polynucleotide encoding said nuclease, described or disclosed in PCT Application Publication WO2024020346A1, filed Jul. 17, 2023, which is incorporated by reference herein in its entirety. In certain embodiments, the LNP further comprises a guide RNA that enables an edit for treating sickle cell anemia or beta-thalassemia.

In certain embodiments, an LNP of the present disclosure comprises (a) a base editor, or a polynucleotide encoding the base editor, wherein the base editor comprises a polynucleotide-programmable nucleotide-binding domain and a deaminase domain; (b) a guide polynucleotide, wherein the guide polynucleotide targets the base editor to a target nucleotide sequence. In certain embodiments, the target nucleotide sequence is a protein coding region responsible for sickle cell anemia or beta-thalassemia. In certain embodiments, the base editor and the guide RNA are any of those described and disclosed in US Application Publication US20210371858A1, which is incorporated by reference herein, in its entirety.

In certain embodiments, an LNP of the present disclosure comprises a system for changing a cytosine to a thymine in the genome of a cell, comprising a first fusion protein and a second fusion protein, or first and second expression constructs for expressing the first and second fusion proteins, respectively, wherein a) the first fusion protein comprises: i) a first zinc finger protein (ZFP) domain that binds to a first sequence in a target genomic region in the cell, and ii) a first portion of a cytidine deaminase polypeptide, wherein the cytidine deaminase is a toxin-derived deaminase (TDD) comprising an amino acid sequence at least 90% identical to SEQ ID NO: 49, 81, 92, 95, 98, 101, 104, 107, 134, 143, 152, 157, 162, 167, 172, 177, 184, 189, 194, 199, 204, 209, 214, or 219 of PCT Publication WO2022067122A1; b) the second fusion protein comprises: i) a second ZFP domain that binds to a second sequence in the target genomic region, and ii) a second portion of the cytidine deaminase polypeptide; c) the first and second portions lack cytidine deaminase activity on their own; and d) binding of the first fusion protein and the second fusion protein to the target genomic region results in dimerization of the first and second portions, wherein the dimerized portions form an active cytidine deaminase capable of changing a cytosine to a thymine in the target genomic region, optionally wherein the cell is a eukaryotic cell, optionally wherein the eukaryotic cell is a mammalian cell or a plant cell, further optionally wherein the mammalian cell is a human cell.

In certain embodiments, an LNP of the present disclosure comprises a pair of zinc finger nucleases (ZFNs), the pair comprising the zinc finger proteins (ZFPs) designated 47773 and 47817, wherein 47773 binds to a target site within SEQ ID NO:23 and 47817 binds to a SEQ ID NO:24 of an endogenous human beta-hemoglobin (Hbb) gene comprising a sickle cell mutation, wherein the pair of ZENs makes a genomic modification in the mutant endogenous Hbb gene and the genomic modification comprises insertion of a donor sequence such that the sickle cell mutation is corrected to wild-type Hbb, wherein the referenced SEQ ID NOs are those disclosed in U.S. Pat. No. 10,435,677.

In certain embodiments, an LNP of the present disclosure comprises a guide RNA and a fusion protein comprising a polynucleotide programmable DNA binding domain and an adenosine deaminase domain, wherein the adenosine deaminase domain comprises an arginine (R) or a threonine (T) at amino acid position 147 of the amino acid sequence, wherein the adenosine deaminase domain has at least 95% sequence identity to the amino acid sequence of SEQ ID NO: 2 of U.S. Pat. No. 11,344,609, and wherein said guide RNA targets said fusion protein to effect a deamination of a nucleobase of the HIBG1/2 promoter in the cell. In certain embodiments, an LNP of the present disclosure comprises a guide RNA and a fusion protein comprising a polynucleotide programmable DNA binding domain and an adenosine deaminase domain, comprising a serine(S) at amino acid position 82, wherein the adenosine deaminase domain has at least 85% sequence identity to the amino acid sequence of SEQ ID NO: 2 of U.S. Pat. No. 11,142,760. In certain embodiments, the polynucleotide programmable DNA binding domain comprises a Cas9 domain. In certain embodiments, the Cas9 domain comprises a dead Cas9 (dCas9) or a nickase Cas9 (nCas9). In certain embodiments, the Cas9 domain is capable of programmable DNA binding and is selected from the group consisting of a Streptococcus pyogenes Cas9 (SpCas9), Staphylococcus aureus Cas9 (SaCas9), and a Streptococcus thermophilus 1 Cas9 (St1Cas9). In certain embodiments, the Cas9 domain comprises an amino acid sequence having at least 85% amino acid sequence identity to the amino acid sequence of SEQ ID NO: 1 of U.S. Pat. No. 11,142,760. In certain embodiments, the fusion protein is selected from ABE8.1-m, ABE8.2-m, ABE8.8-m, ABE8.9-m, ABE8.10-m, ABE8.11-m, ABE8.12-m, ABE8.13-m, ABE8.15-m, ABE8.16-m, ABE8.20-m, ABE.21-m, ABE8.24-m, ABE8.1-d, ABE8.2-d, ABE8.8-d, ABE8.9-d, ABE8.10-d, ABE8.11-d, ABE8.12-d, ABE8.13-d, ABE8.15-d, ABE8. 16-d, ABE8.20-d, ABE.21-d, and ABE8.24-d. In certain embodiments, an LNP of the present disclosure comprises a guide RNA and a base editor comprising a polynucleotide programmable DNA binding domain and a deaminase domain, or a polynucleotide encoding the base editor, wherein the guide RNA is capable of targeting the polynucleotide programmable DNA binding domain to induce a 10 nucleobase change in a target hemoglobin (HBB) gene or in the promoter region of HBG1/2. In certain embodiments, the base editor and the guide RNA are any of those described and disclosed in PCT Publication WO2021163587A1, which is incorporated by reference herein, in its entirety.

In certain embodiments, an LNP of the present disclosure comprises a base editor in complex with one or more guide polynucleotides, wherein the base editor comprises a polynucleotide programmable DNA binding domain and an adenosine deaminase domain, and wherein the one or more guide polynucleotides target the base editor to effect an A·T to G·C alteration of a single nucleotide polymorphism (SNP) associated with sickle cell disease. In certain embodiments, the polynucleotide programmable DNA binding domain is a modified Staphylococcus aureus Cas9 (SaCas9), Streptococcus thermophilus 1 Cas9 (St1Cas9), a modified Streptococcus pyogenes Cas9 (SpCas9), or variants thereof. In certain embodiments, the adenosine deaminase is a TadA deaminase. In certain embodiments, the one or more guide RNAs comprises a CRISPR RNA (crRNA) and a trans-encoded small RNA (tracrRNA), wherein the crRNA comprises a nucleic acid sequence complementary to an HBB nucleic acid sequence comprising the SNP associated with sickle cell disease; or wherein the base editor is in complex with a single guide RNA (sgRNA) comprising a nucleic acid sequence complementary to an HBB nucleic acid sequence comprising the SNP associated with sickle cell disease. In certain embodiments, the base editor and the guide RNA are any of those described and disclosed in US Application Publication US20220401530A1, which is incorporated by reference herein, in its entirety.

In certain embodiments, an LNP of the present disclosure comprises a system for modifying DNA comprising: (a) a polypeptide or a nucleic acid encoding the polypeptide, wherein the polypeptide comprises (i) a reverse transcriptase (RT) domain, (ii) a DNA-binding domain (DBD); and (iii) an endonuclease domain, e.g., a nickase domain; and (b) a template RNA (or DNA encoding the template RNA) comprising (e.g., from 5′ to 3′) (i) optionally a sequence that binds a target site (e.g., a second strand of a site in a target genome), (ii) optionally a sequence that binds the polypeptide, (iii) a heterologous object sequence, and (iv) a 3′ target homology domain, wherein the RT domain comprises a sequence of Table 1 or 3 or a sequence of a reverse transcriptase domain of Table 2, or a sequence having at least 70%, 75%, 80%, 85%, 90%, 95%, 96%, 97%, 98%, or 99% identity thereto, or wherein the RT domain further comprises a number of substitutions relative to the natural sequence, e.g., at least 1, 2, 3, 4, 5, 10, 20, 30, 40, 50, 60, 70, 80, 90, or 100 substitutions (referenced tables being those in PCT Publication WO2021178720A2, which is incorporated by reference herein in its entirety). In certain embodiments, an LNP of the present disclosure comprises a system for modifying DNA comprising: (a) a polypeptide or a nucleic acid encoding the polypeptide, wherein the polypeptide comprises (i) a reverse transcriptase (RT) domain, (ii) a DNA-binding domain (DBD); and (iii) an endonuclease domain, e.g., a nickase domain; and (b) a template RNA (etRNA) (or DNA encoding the template RNA) comprising (e.g., from 5′ to 3′) (i) optionally a sequence that binds a target site (e.g., a second strand of a site in a target genome), (ii) optionally a sequence that binds the polypeptide, (iii) a heterologous object sequence, and (iv) a 3′ target homology domain, wherein the system is capable of producing an insertion into the target site of at least 45, 50, 55, 60, 65, 70, 75, 80, 85, 90, 95, or 100 nucleotides or wherein the heterologous object sequence is at least 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, or 1,000 nts in length. In certain embodiments, the LNP comprises one or more of the systems for modifying DNA disclosed and described in PCT Publication WO2021178720A2, which is incorporated by reference herein in its entirety.

In certain embodiments, an LNP of the present disclosure comprises a system for correcting an E6V mutation in human beta-globin (HBB) in a cell or population of cells, the system comprising: (a) a Cas9 endonuclease, an mRNA encoding the Cas9 endonuclease, or a recombinant expression vector comprising a nucleotide sequence encoding the Cas9 endonuclease; (b) a single guide RNA (sgRNA) comprising a spacer sequence corresponding to a target sequence adjacent to a PAM, the target sequence comprising a target site within intron 1 of HBB; and (c) a recombinant vector comprising a nucleic acid for correcting the E6V mutation, the nucleic acid comprising a nucleotide sequence homologous with a region of the HBB gene encoding the E6V mutation, wherein the nucleotide sequence comprises a codon encoding E6. In certain embodiments, the target sequence comprises a nucleotide sequence selected from SEQ ID NO: 1 or SEQ ID NO: 49 of US Application Publication US20220228142A1. In certain embodiments, the codon encoding E6 is selected from GAA and GAG, and wherein the nucleotide sequence of (c) comprises one or more silent mutations relative to the HBB gene.

In certain embodiments, the nucleotide sequence of (c) comprises a nucleotide sequence having at least 90% sequence identity to a nucleotide sequence selected from SEQ ID NO: 6 or SEQ ID NO: 19 of US Application Publication US20220228142A1. In certain embodiments, the nucleotide sequence of (c) comprises a mutation to delete the PAM. In certain embodiments, the Cas9 endonuclease is a S. pyogenes Cas9 (SpCas9) endonuclease. In certain embodiments, the SpCas9 endonuclease is a high fidelity SpCas9 endonuclease. In certain embodiments, the system comprises the Cas9 endonuclease as a polypeptide, and wherein the system comprises a ribonucleoprotein complex of the sgRNA and the Cas9 endonuclease. In certain embodiments, the system comprises an mRNA encoding the Cas9 endonuclease.

In certain embodiments, an LNP of the present disclosure comprises:
(a) a S. pyogenes Cas9 endonuclease, an mRNA encoding the S. pyogenes Cas9 endonuclease, or a recombinant expression vector comprising a nucleotide sequence encoding the S. pyogenes Cas9 endonuclease, wherein the S. pyogenes Cas9 endonuclease is a high fidelity Cas9;
(b) a single guide RNA (sgRNA) targeting a target site in an HBB gene, the sgRNA comprising a spacer sequence corresponding to a target sequence consisting of SEQ ID NO: 15 of US Patent Application Publication US20210008161A1; and (c) a recombinant vector comprising a nucleic acid, the nucleic acid comprising from 5′ to 3′ (i) a nucleotide sequence homologous with a region located upstream of the target region in the HBB gene, (ii) a nucleotide sequence homologous with a region of the HBB gene comprising the target region, and (iii) a nucleotide sequence homologous with a region located downstream of the target region in the HBB gene; wherein the contents of the LNP are capable of executing a double-strand break (DSB) at the target site in the HBB gene and the nucleic acid is exchanged with a homologous nucleotide sequence of the HBB gene. In certain embodiments, the nucleotide sequence of (c) (ii) corrects an E6V mutation in the HBB gene and is homologous with a region of the HBB gene encoding the E6V mutation. In certain embodiments, the nucleotide sequence of (c) (ii) comprises the sequence of SEQ ID NO: 102 of US Patent Application Publication US20210008161A1.

In certain embodiments, an LNP of the present disclosure comprises a gene editing system disclosed in any one of PCT Publications WO2023019227A1, WO2023019226A1, and WO2023019225A2 which are incorporated by reference herein in their entirety. In certain embodiments the gene editing system is capable of executing a modification in a cell selected from (i) increased expression of one or more tolerogenic factors, (ii) increased expression of CD46, (iii) increased expression of CD59, and (iv) reduced expression of one or more MHC class I molecules and/or one or more MHC class II molecules, wherein the increased expression of (i), (ii), and (iii) and the reduced expression of (iv) is relative to a cell of the same cell type that does not comprise modifications. In certain embodiments, the gene editing system is capable of executing a modification in a cell selected from (a) reduced expression of a MIIC class I chain-related protein A (MICA) and/or a MHC class I chain-related protein B (MICB); (b) increased expression of one or more tolerogenic factors; and (c) reduced expression of one or more major histocompatibility complex class I molecules (MHC class I molecules) and/or one or more major histocompatibility complex class II molecules (MHC class II molecules), wherein the change in expression is relative to a cell of the same cell type that does not comprise modifications. In certain embodiments the gene editing system is capable of executing a modification in a cell selected from (i) increased expression of one or more tolerogenic factor, (ii) reduced expression of CD 142, and (iii) reduced expression of one or more MHC class I molecules and/or one or more MHC class II molecules, wherein the increased expression of (i) and the reduced expression of (ii) and (iii) is relative to a cell of the same cell type that does not comprise modifications.

In certain embodiments, an LNP of the present disclosure comprises a template RNA comprising, from 5′ to 3′: (i) a gRNA spacer that is complementary to a first portion of the human HBB gene, wherein the gRNA spacer has a sequence comprising the core nucleotides of a gRNA spacer sequence of Table 1, and optionally comprises one or more consecutive nucleotides starting with the 3′ end of the flanking nucleotides of the gRNA spacer, or wherein the gRNA spacer has a sequence of a spacer chosen from Table A, Table AA, Table B, Table BI, Tables 5A-5D, Table X4, or Table X4A (referenced tables being those in PCT Publication WO2023039440A2, which is incorporated by reference herein in its entirety); (ii) a gRNA scaffold that binds a gene modifying polypeptide (e.g., binds the Cas domain of the gene modifying polypeptide), (iii) a heterologous object sequence comprising a mutation region to introduce a mutation into (e.g., to correct a mutation in) a second portion of the human HBB gene (wherein optionally the heterologous object sequence comprises, from 5′ to 3′, a post-edit homology region, a mutation region, and a pre-edit homology region), and (iv) a primer binding site (PBS) sequence comprising at least 5, 6, 7, or 8 bases with 100% identity to a third portion of the human HBB gene. In certain embodiments, an LNP of the present disclosure comprises a template RNA comprising, from 5′ to 3′: (i) a gRNA spacer that is complementary to a first portion of the human HBB gene, (ii) a gRNA scaffold that binds a gene modifying polypeptide (e.g., binds the Cas domain of the gene modifying polypeptide), (iii) a heterologous object sequence comprising a mutation region to introduce a mutation into a second portion of the human HBB gene, wherein the heterologous object sequence comprises the core nucleotides of an RT template sequence of Table 3, and optionally comprises one or more consecutive nucleotides starting with the 3′ end of the flanking nucleotides of the RT template sequence, or wherein the heterologous object sequence comprises an RT template sequence of Table A, Table AA, Table B, Table BI, Tables 5A-5D, Table X4, or Table X4A (referenced tables being those in PCT Publication WO2023039440A2, which is incorporated by reference herein in its entirety); and (iv) a PBS sequence comprising at least 5, 6, 7, or 8 bases of 100% identity to a third portion of the human IIBB gene.

In certain embodiments, an LNP of the present disclosure comprises a guide RNA (gRNA) comprises, from 5′ to 3′, [targeting domain]-(SEQ ID NO: 195 or 231), wherein the targeting domain comprises SEQ ID NO: 67, wherein the referred to SEQ ID NOs are those described in U.S. Pat. No. 11,466,271. In certain embodiments, said SEQ ID NO: 195 or 231 is disposed immediately 3′ to said targeting domain. In certain embodiments, the gRNA comprises at the 3′ end, 1, 2, 3, 4, 5, 6 or 7 uracil (U) nucleotides. In certain embodiments, the LNP further comprises a Cas9 or a polynucleotide encoding the same. In certain embodiments, the LNP further comprises a template nucleic acid. In certain embodiments, the Cas9 comprises a sequence of SEQ ID NO: 205 of U.S. Pat. No. 11,466,271, or a sequence with at least 95% homology thereto. In certain embodiments, the Cas9 comprises a sequence selected from SEQ ID NOs: 233-244 of U.S. Pat. No. 11,466,271. In certain embodiments, the gRNA comprises or consists of the sequence of SEQ ID NOs: 174-176 of U.S. Pat. No. 11,466,271. In certain embodiments, an LNP of the present disclosure comprises any gRNA, nuclease and/or template nucleic acid (or a polynucleotide encoding any of the aforementioned) disclosed in U.S. Pat. No. 11,466,271, which is incorporated by reference herein, in its entirety.

In certain embodiments, an LNP of the present disclosure comprises a gRNA comprising a tracr and crRNA, wherein the crRNA comprises a targeting domain that is complementary with a target sequence of a BCL11A gene (e.g., a human BCL11a gene), a BCL11a enhancer (e.g., a human BCL11a enhancer), or a HFPH region (e.g., a human HPFH region). In certain embodiments, the target sequence is of the BCL11A gene, and the targeting domain comprises, e.g., consists of, any one of SEQ ID NO: 1 to SEQ ID NO: 85 or SEQ ID NO: 400 to SEQ ID NO: 1231 of US Application Publication US20190010495A1. In certain embodiments, the target sequence is of a BCL11a enhancer, and the targeting domain comprises, e.g., consists of, any one of SEQ ID NO: 1232 to SEQ ID NO: 1499 of US Application Publication US20190010495A1. In certain embodiments, the target sequence is of a BCL11a enhancer, and the targeting domain comprises, e.g., consists of, any one of SEQ ID NO: 182 to SEQ ID NO: 277 or SEQ ID NO: 334 to SEQ ID NO: 341 of US Application Publication US20190010495A1. In certain embodiments, the targeting domain comprises, e.g., consists of, any one of SEQ ID NO: 341, SEQ ID NO: 246, SEQ ID NO: 248, SEQ ID NO: 247, SEQ ID NO: 245, SEQ ID NO: 249, SEQ ID NO: 244, SEQ ID NO: 199, SEQ ID NO: 251, SEQ ID NO: 250, SEQ ID NO: 334, SEQ ID NO: 185, SEQ ID NO: 186, SEQ ID NO: 336, or SEQ ID NO: 337 of US Application Publication US20190010495A1. In certain embodiments, the targeting domain comprises, e.g., consists of, any one of SEQ ID NO: 247, SEQ ID NO: 248, SEQ ID NO: 335, SEQ ID NO: 336, SEQ ID NO: 337, or SEQ ID NO: 338 of US Application Publication US20190010495A1. In certain embodiments, the targeting domain comprises, e.g., consists of, any one of SEQ ID NO: 248 or SEQ ID NO: 338 of US Application Publication US20190010495A1. In certain embodiments, the targeting domain comprises, e.g., consists of, SEQ ID NO: 248 of US Application Publication US20190010495A1. In certain embodiments, the targeting domain comprises, e.g., consists of, SEQ ID NO: 338 of US Application Publication US20190010495A1. In certain embodiments, the target sequence is of a BCL11a enhancer, and the targeting domain comprises, e.g., consists of, any one of SEQ ID NO: 278 to SEQ ID NO: 333 of US Application Publication US20190010495A1. In certain embodiments, the targeting domain comprises, e.g., consists of, any one of SEQ ID NO: 318, SEQ ID NO: 312, SEQ ID NO: 313, SEQ ID NO: 294, SEQ ID NO: 310, SEQ ID NO: 319, SEQ ID NO: 298, SEQ ID NO: 322, SEQ ID NO: 311, SEQ ID NO: 315, SEQ ID NO: 290, SEQ ID NO: 317, SEQ ID NO: 309, SEQ ID NO: 289, or SEQ ID NO: 281 of US Application Publication US20190010495A1. In certain embodiments, the targeting domain comprises, e.g., consists of, SEQ ID NO: 318 of US Application Publication US20190010495A1. In certain embodiments, the target sequence is of a BCL11a enhancer, and the targeting domain comprises, e.g., consists of, any one of SEQ ID NO: 1596 to SEQ ID NO: 1691 of US Application Publication US20190010495A1. In certain embodiments, the targeting domain comprises, e.g., consists of, any one of SEQ ID NO: 1683, SEQ ID NO: 1638, SEQ ID NO: 1647, SEQ ID NO: 1609, SEQ ID NO: 1621, SEQ ID NO: 1617, SEQ ID NO: 1654, SEQ ID NO: 1631, SEQ ID NO: 1620, SEQ ID NO: 1637, SEQ ID NO: 1612, SEQ ID NO: 1656, SEQ ID NO: 1619, SEQ ID NO: 1675, SEQ ID NO: 1645, SEQ ID NO: 1598, SEQ ID NO: 1599, SEQ ID NO: 1663, SEQ ID NO: 1677, or SEQ ID NO: 1626 of US Application Publication US20190010495A1. In certain embodiments, the target sequence is of a HFPH region (e.g., a French HPFH region), and the targeting domain comprises, e.g., consists of, any one of SEQ ID NO: 86 to SEQ ID NO: 181, SEQ ID NO: 1500 to SEQ ID NO: 1595, or SEQ ID NO: 1692 to SEQ ID NO: 1761 of US Application Publication US20190010495A1. In certain embodiments, the targeting domain comprises, e.g., consists of, any one of SEQ ID NO: 100, SEQ ID NO: 165, SEQ ID NO: 113, SEQ ID NO: 99, SEQ ID NO: 112, SEQ ID NO: 98, SEQ ID NO: 1580, SEQ ID NO: 106, SEQ ID NO: 1503, SEQ ID NO: 1589, SEQ ID NO: 160, SEQ ID NO: 1537, SEQ ID NO: 159, SEQ ID NO: 101, SEQ ID NO: 162, SEQ ID NO: 104, SEQ ID NO: 138, SEQ ID NO: 1536, SEQ ID NO: 1539, SEQ ID NO: 1585 of US Application Publication US20190010495A1. In certain embodiments, the targeting domain comprises, e.g., consists of, any one of SEQ ID NO: 98, SEQ ID NO: 100, SEQ ID NO: 1505, SEQ ID NO: 1589, SEQ ID NO: 1700, or SEQ ID NO: 1750 of US Application Publication US20190010495A1. In certain embodiments, the targeting domain comprises, e.g., consists of, any one of SEQ ID NO: 100, SEQ ID NO: 165, or SEQ ID NO: 113. In certain embodiments, an LNP of the present disclosure comprises any gRNA, nuclease and/or template nucleic acid (or a polynucleotide encoding any of the aforementioned) disclosed in US Application Publication US20190010495A1, which is incorporated by reference herein, in its entirety.

In certain embodiments, an LNP of the present disclosure comprises one or more S. pyogenes Cas9 deoxyribonucleic acid (DNA) endonucleases and one or two ribonucleic acid (RNA) guides capable of effecting a pair of double-strand breaks (DSBs), the first at a 5′ DSB locus and the second at a 3′ DSB locus within the 8 3-globin region of human chromosome 11, causing a deletion or inversion of the chromosomal DNA between the 5′ DSB locus and the 3′ DSB locus that results in increased expression of γ-globin. In certain embodiments, an LNP of the present disclosure comprises one or more S. pyogenes Cas9 deoxyribonucleic acid (DNA) endonucleases and one or two ribonucleic acid (RNA) guides capable of effecting a pair of double-strand breaks (DSBs), the first at a 5′ DSB locus and the second at a 3′ DSB locus within the δβ-globin region of human chromosome 11, causing a deletion or inversion of the chromosomal DNA between the 5′ DSB locus and the 3′ DSB locus. In certain embodiments, the first RNA guide comprises a spacer sequence that is complementary to a segment of the 5′ DSB locus, and the second RNA guide comprises a spacer sequence that is complementary to a segment of the 3′ DSB locus. In certain embodiments, the 5′ DSB locus is proximal to the 5′ boundary of an HPFH deletion. In certain embodiments, the 5′ DSB locus is proximal to the 5′ boundary of an HPFH deletion selected from the group consisting of the HPFH-4 deletion, the HPFH-5 deletion, the HPFH-Kenya deletion, the HPFH-Black deletion, the long Corfu deletion, and the short Corfu deletion. In certain embodiments, the 3′ DSB locus is proximal to the 3′ boundary of an HPFH deletion. In certain embodiments, the 3′ DSB locus is proximal to the 3′ boundary of an HPFH deletion selected from the group consisting of the HPFH-4 deletion, the HPFH-5 deletion, the HPFH-Kenya deletion, the HPFH-Black deletion, the long Corfu deletion, and the short Corfu deletion. In certain embodiments, the endonuclease and the guide RNA are any of those described and disclosed in US20180030438A1 and/or U.S. Pat. No. 10,738,305, both of which are incorporated by reference herein, in their entirety. In certain embodiments, the LNP comprises a polynucleotide encoding the endonuclease. In certain embodiments, an LNP of the present disclosure comprises a DNA endonuclease capable of effecting a double-strand break (DSB) at one or more loci within the δβ-globin region of human chromosome 11, causing deletions or insertions of chromosomal DNA at the one or more loci that results in increased expression of γ-globin, thereby increasing the level of HbF in the cell, wherein one of the loci comprises a sequence that is complementary to a nucleic acid sequence of SEQ ID NO: 139 of U.S. Pat. No. 10,738,305. In certain embodiments, the DNA endonuclease is a Cas9 endonuclease and the method comprises introducing into the cell one or more polynucleotides encoding Cas9 and one or more guide RNAs, each comprising a spacer sequence that is complementary to the one or more loci within the β-globin region of human chromosome 11. In certain embodiments, the one or more guide RNA comprises a spacer sequence that is complementary to positions 51-69 of the nucleic acid sequence of SEQ ID NO: 180 of U.S. Pat. No. 10,738,305. In certain embodiments, the one or more guide RNA comprises a spacer sequence that hybridizes to the same target sequence of the δβ-globin region of human chromosome 11 as a nucleic acid having the nucleic acid sequence of SEQ ID NO: 139 of U.S. Pat. No. 10,738,305.

In certain embodiments, an LNP of the present disclosure comprises a DNA endonuclease capable of effecting a pair of double-strand breaks (DSBs), the first at a 5′ DSB locus and the second at a 3′ DSB locus within the δβ-globin region of human chromosome 11, causing a deletion or inversion of the chromosomal DNA between the 5′ DSB locus and the 3′ DSB locus. In certain embodiments, the DNA endonuclease is a Cas9 endonuclease, a zinc finger nuclease, a transcription activator-like effector nuclease, a homing endonuclease, a dCas9-FokI nuclease or a MegaTal nuclease. In certain embodiments, the LNP comprises a polynucleotide encoding the endonuclease. In certain embodiments, the LNP comprises two guide RNAs, the first guide RNA comprising a spacer sequence that is complementary to a segment of the 5′ DSB locus, and the second guide RNA comprising a spacer sequence that is complementary to a segment of the 3′ DSB locus. In certain embodiments, both guide RNAs are single-molecule gRNAs. In certain embodiments, the endonuclease and the guide RNA are any of those described and disclosed in US20180200387A1, which is incorporated by reference herein, in its entirety. In certain embodiments, the endonuclease and the guide RNA are any of those described and disclosed in US20180273609A1, which is incorporated by reference herein, in its entirety.

In certain embodiments, an LNP of the present disclosure comprises one or more base editors, or a polynucleotide encoding the same, and a guide RNA bound to the base editor, wherein the guide RNA (gRNA) comprises a guide sequence that is complementary to a target nucleic acid sequence in the promoter of the HBG1 and/or HBG2 gene. In certain embodiments, the guide sequence comprises at least 15, 16, 17, 18, 19, 20, 21, 22, 23, 24, or 25 contiguous nucleobases that are 100% complementary to the target nucleic acid sequence of the promoter. In certain embodiments, the target nucleic acid comprises one of SEQ ID NOs: 838-845 of US20200399626A1. In certain embodiments, the target nucleic acid sequence further comprises 5′-CCT-3′ at the 5′ end. In certain embodiments, the target nucleic acid sequence comprises SEQ ID NO: 842 of US20200399626A1. In certain embodiments, the guide sequence comprises one of SEQ ID NOs: 846-853 of US20200399626A1. In certain embodiments, the guide sequence comprises one of SEQ ID NOs: 254-291 of US20200399626A1. In certain embodiments, the target nucleic acid comprises one of SEQ ID NOs: 297-302 of US20200399626A1. In certain embodiments, the guide sequence comprises SEQ ID NO: 260 of US20200399626A1. In certain embodiments, the target sequence comprises SEQ ID NO: 303 of US20200399626A1. In certain embodiments, the guide sequence comprises one of SEQ ID NOs: 261-266 of US20200399626A1. In certain embodiments, the target nucleic acid comprises one of SEQ ID NOs: 304-309 of US20200399626A1. In certain embodiments, the guide sequence comprises SEQ ID NO: 267 of US20200399626A1. In certain embodiments, the target sequence comprises SEQ ID NO: 310 of US20200399626A1. In certain embodiments, the target nucleic acid comprises one of SEQ ID NOs: 297-323 of US20200399626A1. In certain embodiments, the LNP comprises a second guide RNA.

In certain embodiments, an LNP of the present disclosure comprises one or more deoxyribonucleic acid (DNA) endonucleases capable of effecting two or more single-strand breaks (SSB) or double-strand breaks (DSB), a first SSB or DSB within or near a human beta globin locus on chromosome 11, and a second SSB or DSB within or near one or more of: a BCL11A gene on chromosome 2, and a DNA sequence that encodes a transcriptional control region of the BCL11A gene on chromosome 2; wherein the first SSB or DSB results in one or more of: a permanent deletion or inversion within or near the human beta globin locus on chromosome 11, and a mutation within or near the human beta globin locus on chromosome 11; wherein the second SSB or DSB results in one or more of: a permanent deletion or inversion within or near the BCL11A gene on chromosome 2, a mutation within or near the BCL11A gene on chromosome 2, a permanent deletion or inversion within or near the DNA sequence that encodes a transcriptional control region of the BCL11A gene on chromosome 2, and a mutation within or near the DNA sequence that encodes a transcriptional control region of the BCL11A gene on chromosome 2; and wherein the two or more permanent deletions, inversions, or mutations result in an increased expression of γ-globin and an increase of fetal hemoglobin (HbF) in the human cell. In certain embodiments, the permanent deletion within or near the human beta globin locus on chromosome 11 is a hereditary persistence of fetal hemoglobin (HPFH) deletion. In certain embodiments, the HPFH deletion is selected from the group consisting of: a HPFH-4 deletion, a HPFH-5 deletion, a HPFH-Kenya deletion, a HPFH-Black deletion, a large Corfu deletion, a small Corfu deletion, a small deletion, and combinations thereof. In certain embodiments, the HPFH deletion is a 7.2 kb, 3.5 kb, 12.9 kb, 22 kb, or a 13 bp deletion. In certain embodiments, the one or more DNA endonuclease is selected from the group consisting of a zinc finger nuclease, TAL effector nuclease, meganuclease, MegaTAL, Tev-m TALEN, MegaTev, homing endonuclease, FokI, StsI, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, a homolog thereof, a recombination of the naturally occurring molecule thereof, a codon-optimized thereof, modified versions thereof, and combinations thereof. In certain embodiments, the LNP further comprises one or more guide ribonucleic acid (gRNA), optionally comprising one or more of the gRNA sequences provided in Table 2 of US20210180091A1, optionally wherein the one or more gRNAs comprise the sequence of SPY101 in combination with the sequence of any one or more of HPFH5-1, HPFH5-D, HPFH5-T5, HPFH5-T7, Kenya-K5, or Kenya-K1; or optionally wherein the one or more gRNAs comprise the sequence of 1450 in combination with the sequence of any one or more of HPFH5-1, HPFH5-D, HPFH5-T5, HPFH5-T7, Kenya-K5, or Kenya-K1; or optionally wherein the one or more gRNAs comprise the sequence of SPY101 in combination with the sequence of SD2; or optionally wherein the one or more gRNAs comprise the sequence of 1450 in combination with the sequence of SD2; or optionally wherein the one or more gRNAs comprise the sequence of HPFH5-1, HPFH5-D, HPFH5-T5, HPFH5-T7, Kenya-K5, or Kenya-K1 in combination with the sequence of any one or more of HPFH5-1, HPFH5-D, HPFH5-T5, HPFH5-T7, Kenya-K5, or Kenya-K1. In certain embodiments, the one or more DNA endonucleases and/or gRNAs are selected from those disclosed in US20210180091A1, which is incorporated by reference herein, in its entirety.

In certain embodiments, an LNP of the present disclosure comprises a genome editing system comprising an RNA-guided nuclease; a first guide RNA; and a second guide RNA, wherein the first and second guide RNAs comprise first and second targeting domains complimentary to first and second sequences on opposite sides of positions of a 13 nt target region of a human HBG1 or HBG2 gene, wherein one or both of the first and second sequences optionally overlaps the 13 nt target region of the human HBG1 or HBG2 gene. In certain embodiments, the genome editing system further comprises a nucleic acid template encoding a deletion of the 13 nt region of a human HBG1 or HBG2 gene. In certain embodiments, the RNA-guided nuclease is an S. pyogenes Cas9. In certain embodiments, the first and second targeting domains are complimentary to sequences immediately adjacent to a protospacer adjacent motif recognized by S. pyogenes Cas9. In certain embodiments, the RNA-guided nuclease is a nickase, and optionally lacks RuvC activity. In certain embodiments, the first targeting domain is complimentary to a sequence within positions c. −1,114 to −114 of a human HBG1 or HBG2 gene. In certain embodiments, the first targeting domain is complimentary to a sequence within positions c. −214 to −114 of a human HIBG1 or IIBG2 gene. In certain embodiments, one of the first and second targeting domains is complimentary to a sequence within positions c. −102 to −52 of a human HBG1 or HBG2 gene. In certain embodiments, the second targeting domain is complimentary to a sequence within positions c. −102 to −2 of a human HBG1 or HBG2 gene. In certain embodiments, at least one of the first and second targeting domains differ by no more than 3 nucleotides from a targeting domain listed in Table 7 of US Patent Application Publication US20220047637A1. In certain embodiments, the genome editing system comprises first and second RNA-guided nucleases. In certain embodiments, the first and second RNA-guided nucleases are complexed with the first and second guide RNAs, respectively, forming first and second ribonucleoprotein complexes. In certain embodiments, the genome editing system further comprises: a third guide RNA; and optionally a fourth guide RNA, wherein the third and fourth guide RNAs comprise third and fourth targeting domains complimentary to third and fourth sequences on opposite sides of positions of a GATA1 binding motif in BCL11A erythroid enhancer (BCL11Ae) of a human BCL11A gene, wherein one or both of the third and fourth sequences optionally overlaps the GATA1 binding motif in BCL11Ae of the human BCL11A gene. In certain embodiments, the genome editing system further comprises a nucleic acid template encoding a deletion of the GATA1 binding motif in BCL11Ae. In certain embodiments, the third targeting domain is complimentary to a sequence within 1000 nucleotides upstream of the GATA1 binding motif in BCL11Ae. In certain embodiments, the third targeting domain is complimentary to a sequence within 100 nucleotides upstream of the GATA1 binding motif in BCL11Ae. In certain embodiments, one of the third and fourth targeting domains is complimentary to a sequence within 100 nucleotides downstream of the GATA1 binding motif in BCL11Ae. In certain embodiments, the fourth targeting domain is complimentary to a sequence within 50 nucleotides downstream of the GATA1 binding motif in BCL11Ae. In certain embodiments, at least one of the third and fourth targeting domains differ by no more than 3 nucleotides from a targeting domain listed in Table 9 of US Patent Application Publication US20220047637A1.

In certain embodiments, an LNP of the present disclosure comprises a guide RNA (gRNA) comprising: a 5′ end and a 3′ end, a DNA extension at the 5′ end, a 2′-O-methyl-3′-phosphorothioate modification at the 3′ end, a targeting domain that is complementary to a target site in a promoter of an HBG gene, and an RNA segment capable of associating with a Cpf1 RNA-guided nuclease. In certain embodiments, the DNA extension comprises a sequence selected from the group consisting of SEQ ID NOs: 1235-1250 as disclosed in US Patent Application Publication US20210254061A1. In certain embodiments, the targeting domain comprises a sequence set forth in Table 13, Table 18, or Table 19 of US Patent Application Publication US20210254061A1. In certain embodiments, the target site comprises nucleotides located between Chr 11 (NC_000011.10) 5,249,904-5,249,927 (Table 17, Region 6, of US Patent Application Publication US20210254061A1); Chr 11 (NC_000011.10) 5,254,879-5,254,909 (Table 17, Region 16, of US Patent Application Publication US20210254061A1); or a combination thereof. In certain embodiments, an LNP of the present disclosure comprises a ribonucleoprotein (RNP) complex comprising a Cpf1 variant and a gRNA as described above. In certain embodiments, the Cpf1 variant contains one or more modifications selected from the group consisting of one or more mutations in a wild-type Cpf1 amino acid sequence, one or more mutations in a wild-type Cpf1 nucleic acid sequence, one or more nuclear localization signals, one or more purification tags, and a combination thereof. In certain embodiments, the Cpf1 variant is encoded by a sequence selected from the group consisting of SEQ ID NOs: 1000, 1001, 1008-1018, 1032, 1035-39, 1094-1097, and 1107-09 of US Patent Application Publication US20210254061A1. In certain embodiments, the Cpf1 variant is encoded by a sequence selected from the group consisting of SEQ ID NOs: 1019-1021 and 1110-1117 of US Patent Application Publication US20210254061A1.

In certain embodiments, an LNP of the present disclosure comprises a fusion protein comprising a polynucleotide programmable DNA binding domain and at least one adenosine deaminase variant comprising a serine(S) at amino acid position 82 of the following sequence and having at least 85% identity to SEQ ID NO: 4 of U.S. Pat. No. 11,155,803. In certain embodiments, the adenosine deaminase variant further comprises an alteration at amino acid position 166. In certain embodiments, the adenosine deaminase variant further comprises a T166R alteration. In certain embodiments, the adenosine deaminase variant further comprises one or more of the following alterations: Y147T, Y147R, Q154S, Y123H, and Q154R. In certain embodiments, the adenosine deaminase variant further comprises an alteration or combination of alterations selected from the group consisting of: Y147T and Q154R; Y147T and Q154S; Y147R and Q154S; Q154S; Y147R; Q154R; Y123H; I76Y; Y123H, and Y147T; Y123H, and Y147R; Y123H, and Q154R; Y147R, Q154R, and Y123H; Y147R, Q154R, and I76Y; Y147R, Q154R, and T166R; Y123H, Y147R, Q154R, and I76Y; Y123H, Y147R, and Q154R; and I76Y, Y123H, Y147R, and Q154R. In certain embodiments, the fusion protein further comprises a second adenosine deaminase variant or a second adenosine deaminase. In certain embodiments, the fusion protein is ABE8.5-m, ABE8.14-m, ABE8.15-m, ABE8.16-m, ABE8.17-m, ABE8.18-m, ABE8.19-m, ABE8.20-m, ABE8.22-m, ABE8.23-m, or ABE8.24-m. In certain embodiments, the second adenosine deaminase comprises a wild-type adenosine deaminase. In certain embodiments, the fusion protein is ABE8.5-d, ABE8.14-d, ABE8.15-d, ABE8.16-d, ABE8.17-d, ABE8.18-d, ABE8.19-d, ABE8.20-d, ABE8.22-d, ABE8.23-d, or ABE8.24-d. In certain embodiments, the adenosine deaminase variant is TadA*8.14, TadA*8.15, TadA*8.16, TadA*8.17, TadA*8.18, TadA*8.19, TadA*8.20, TadA*8.22, TadA*8.23, or TadA*8.24. In certain embodiments, the adenosine deaminase variant comprises a deletion of 1, 2, 3, 4, 5, 6, 7, or 8 N-terminal or C-terminal amino acid residues relative to the full-length adenosine deaminase. In certain embodiments, the polynucleotide programmable DNA binding domain comprises SEQ ID NO: 12 of U.S. Pat. No. 11,155,803. In certain embodiments, the polynucleotide programmable DNA binding domain comprises a Cas9 domain. In certain embodiments, the polynucleotide programmable DNA binding domain comprises the SEQ ID NO: 1 of U.S. Pat. No. 11,155,803. In certain embodiments, the Cas9 domain is a Staphylococcus aureus Cas9 (SaCas9), Streptococcus thermophilus 1 Cas9 (St1Cas9), a Streptococcus pyogenes Cas9 (SpCas9), or variants thereof. In certain embodiments, the polynucleotide programmable DNA binding domain is a nuclease inactive or nickase variant. In certain embodiments, the fusion protein comprises a linker between the polynucleotide programmable DNA binding domain and the adenosine deaminase variant. In certain embodiments, the fusion protein comprises one or more nuclear localization signals. In certain embodiments, the adenosine deaminase variant has at least 90% identity to the sequence. In certain embodiments, the adenosine deaminase variant has at least 95% identity to the sequence. In certain embodiments, an LNP of the present disclosure comprises a polynucleotide encoding the fusion protein of any of the above described embodiments.

In certain embodiments, an LNP of the present disclosure comprises a guide RNA molecule comprising a targeting domain comprising a nucleotide sequence complementary or partially complementary with a target sequence encompassing or proximal to position −175 of a 5′ UTR and/or −228 of a transcription start site of HBG1 or HBG2. In certain embodiments, the target sequence is between −150 and −250 of the proximal promoter regions. In certain embodiments, the targeting domain is configured to guide a nucleobase modification at position −175. In certain embodiments, the nucleobase modification results in a T->C mutation. In certain embodiments, an LNP of the present disclosure comprises a nucleic acid composition, comprising: a nucleotide sequence encoding a guide RNA molecule comprising a targeting domain comprising a nucleotide sequence complementary or partially complementary with a target sequence encompassing or proximal to position −175 of a proximal promoter region of HBG1 or HBG2. In certain embodiments, the nucleic acid composition further comprises a nucleotide sequence encoding a base editing protein or peptide. In certain embodiments, the base editing protein or peptide is an adenine base editor (ABE). In certain embodiments, the ABE is selected from the group consisting of ABE7.10, ABE8e, and an ABE comprising SpRY. In certain embodiments, the base editing protein or peptide comprises an adenosine deaminase, optionally an AB Emax protein or peptide. In certain embodiments, the base editing protein or peptide is configured to introduce a T->C mutation in or near the target sequence of the guide RNA. In certain embodiments, the nucleic acid is a viral vector genome or an mRNA. In certain embodiments, an LNP of the present disclosure comprises an isolated ribonucleoprotein complex, comprising: a guide RNA molecule comprising a targeting domain comprising a nucleotide sequence complementary or partially complementary with a target sequence encompassing or proximal to position −175 of a proximal promoter region of HBG1 or HBG2, and a base editor protein or peptide. In any of the above embodiments, the base editing system is configured to introduce a T->C mutation at one or more of: HBG2: GRCh/hg19; chrl 1:5,276,186.; and HBG1: GRCh/hg19; chrl 1:5,271,262. In certain embodiments, the gRNA and the base editing protein or peptide, or nucleotide sequence encoding the same, is any one disclosed in PCT Application Publication WO2022155458A1, which is incorporated by reference herein, in its entirety.

In certain embodiments, an LNP of the present disclosure comprises a ribonucleoprotein (RNP) complex comprising:
(a) a Cas9 endonuclease protein; and
(b) a guide RNA (gRNA) comprising a targeting domain comprising a first nucleotide sequence that is complementary or partially complementary with a target domain comprising a second nucleotide sequence that is:
(i) within nucleotides 1-4758 of SEQ ID NO:902, nucleotides 1-4773 of SEQ ID NO:903, or both; or
(ii) complementary to a sequence within nucleotides 1-4758 of SEQ ID NO:902, nucleotides 1-4773 of SEQ ID NO:903, or both;
wherein any of the sequences referenced are those disclosed in US Patent Application Publication US20200255857A1.

In certain embodiments, the second nucleotide sequence is: (i) within nucleotides 1-2990 of SEQ ID NO:902, nucleotides 1-2914 of SEQ ID NO:903, or both; or (ii) complementary to a sequence within nucleotides 1-2990 of SEQ ID NO:902, nucleotides 1-2914 of SEQ ID NO:903, or both, wherein any of the sequences referenced are those disclosed in US Patent Application Publication US20200255857A1. In certain embodiments, the second nucleotide sequence is: (i) within nucleotides 2424-3236 of SEQ ID NO:902, nucleotides 2348-3160 of SEQ ID NO:903, or a combination thereof or (ii) complementary to a sequence within nucleotides 2424-3236 of SEQ ID NO: 902, nucleotides 2348-3160 of SEQ ID NO:903, or a combination thereof, wherein any of the sequences referenced are those disclosed in US Patent Application Publication US20200255857A1. In certain embodiments, the targeting domain comprises a nucleotide sequence that is identical to, or differs by no more than 1, 2, 3, 4, or 5 nucleotides, from a nucleotide sequence set forth in any of SEQ ID NOs: 251-901, wherein any of the sequences referenced are those disclosed in US Patent Application Publication US20200255857A1. In certain embodiments, the gRNA comprises one or more modifications selected from the group consisting of a 2′-acetylation, a 2′-methylation, and a phosphorothioate modification. In certain embodiments, the Cas9 endonuclease protein is selected from the group consisting of a S. pyogenes, S. aureus, and S. thermophilus Cas9 endonuclease protein

In certain embodiments, an LNP of the present disclosure comprises a gRNA molecule comprising a targeting domain which is complementary with a target domain from the HBB or BCL11A gene. In certain embodiments, an LNP of the present disclosure comprises a nucleotide sequence that encodes a gRNA molecule comprising a targeting domain which is complementary with an SCD target domain in the HBB or BCL11A gene. In certain embodiments, the targeting domain is configured to provide a cleavage event selected from a double strand break and a single strand break, within 500, 400, 300, 200, 100, 50, 25, or 10 nucleotides of an SCD target point position or an SCD target knockout position. In certain embodiments, an LNP of the present disclosure comprises:
(a) a first gRNA molecule comprising a first targeting domain which is complementary with a first target domain located in, or within, a BCL11A gene, wherein the first targeting domain is configured to provide a first cleavage event in a region of the BCL11A gene which is complementary to a sequence that is the same as, or differs by no more than 3 nucleotides from, a sequence selected from the group consisting of SEQ ID NOs: 16261 to 16279 of U.S. Pat. No. 11,242,525; and
(b) a Cas9 molecule, or a polynucleotide encoding the same.

In certain embodiments, the LNP further comprises a second gRNA. In certain embodiments, the second gRNA molecule comprises a second targeting domain which is complementary with a second target domain located in a BCL11A gene, wherein the second targeting domain is configured to provide a second cleavage event in a region of the BCL11A gene, and wherein the second targeting domain comprises a sequence that is the same as, or differs by no more than 3 nucleotides from, a sequence selected from the group consisting of SEQ ID NOs: 16261 to 16279 of U.S. Pat. No. 11,242,525. In certain embodiments, the LNP further comprises a third gRNA, and optionally a fourth gRNA.

In certain embodiments, an LNP of the present disclosure comprises:
1a) a gRNA molecule comprising a targeting domain which is complementary with a target sequence from a target nucleic acid;
1b) one or more Cas9 molecules or one or more nucleic acids encoding one or more Cas9 molecules; and
1c) optionally, a template nucleic acid; or
2a) a nucleic acid encoding the gRNA molecule;
2b) one or more Cas9 molecules or one or more nucleic acids encoding one or more Cas9 molecules; and
2c) optionally, a template nucleic acid.

In certain embodiments, the one or more Cas9 molecules comprise a first caCas9 and a second eaCas9. In certain embodiments, the one or more nucleic acids encoding one or more Cas9 molecules comprise one or more nucleic acids encoding a first eaCas9 molecule and a second eaCas9 molecule. In certain embodiments, the composition comprises a first nucleic acid comprising a first promoter operably linked to a sequence encoding the gRNA. In certain embodiments, the LNP comprises a second nucleic acid comprising a second promoter operably linked to a sequence encoding a second gRNA. In certain embodiments, the LNP comprises a first nucleic acid comprising a first promoter operably linked to a sequence encoding the first eaCas9 and a second nucleic acid comprises a second promoter operably linked to a sequence encoding the second eaCas9. In certain embodiments, the targeting domain comprises a core domain and is 15 to 50 nucleotides in length. In certain embodiments, the gRNA has a structure of 5′-targeting domain-first complementarity domain-linking domain-second complementarity domain-proximal domain-3′. In certain embodiments, the first complementarity domain is 5 to 25 nucleotides in length. In certain embodiments, the proximal domain is 5 to 20 nucleotides in length. In certain embodiments, the linking domain is 1 to 5 nucleotides in length. In certain embodiments, the second complementarity domain is complementary to the first complementarity domain. In certain embodiments, any of the above embodiment are formulated such that the gRNA and the one or more Cas9 molecules, or one or more nucleic acids encoding one or more Cas9 molecules, are formulated in separate LNP formulations. In certain embodiments, the contents of the LNP(s) induce exon skipping. In certain embodiments, the gRNA comprises or consists of any guide RNA sequence, or any portion thereof, disclosed in US Application Publication US20210040506A1, which is incorporated by reference herein in its entirety. In certain embodiments, the LNP further comprises a CRISPR Cas9, or a polynucleotide encoding the same, disclosed in US Application Publication US20210040506A1, which is incorporated by reference herein in its entirety.

In certain embodiments, an LNP of the present disclosure comprises a polypeptide comprising a homing endonuclease (HE) variant that cleaves a target site in the human BCL11A gene, or a polynucleotide encoding such a polypeptide. In certain embodiments, the HE variant is an LAGLIDADG homing endonuclease (LHE) variant. In certain embodiments, the HE variant is a variant of an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NerII, I-NerI, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdil41I. In certain embodiments, HE variant comprises one or more amino acid substitutions at amino acid positions selected from the group consisting of: 19, 24, 26, 28, 30, 32, 34, 35, 36, 37, 38, 40, 42, 44, 46, 48, 68, 70, 72, 75, 76, 77, 78, 80, 82, 168, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5 of US Patent Application Publication US20190184035A1, or a biologically active fragment thereof. In certain embodiments, the IIE variant comprises the amino acid sequence set forth in any one of SEQ ID NOs: 6-19 of US Patent Application Publication US20190184035A1. In certain embodiments, the LNP further comprises a donor repair template.

In certain embodiments, an LNP of the present disclosure comprises one or more polynucleotides encoding a zinc finger nuclease (ZFN) or a TAL-effector domain nuclease (TALEN) that binds to a target site in a BCL11A gene as shown in any of SEQ ID Nos: 58-61, 65, 74 or 77 of U.S. Pat. No. 10,619,154. In certain embodiments, the encoded nuclease cleaves and genetically modifies the BCL11A gene and expression of the globin gene is altered. In certain embodiments, an LNP of the present disclosure comprises a DNA binding molecule comprising a TALE-effector protein (TALE), or a polynucleotide encoding said TALE, or a single guide RNA that binds to a target site as shown in any of SEQ ID Nos: 58-62, 65 or 74-77 of U.S. Pat. No. 10,619,154.

In certain embodiments, an LNP of the present disclosure comprises SB-mRENH1 mRNAs and SB-mRENH2 mRNAs, which encode a ZFN pair; wherein the ZFN pair is capable of making a genomic modification within an endogenous BCL11A enhancer sequence. In certain embodiments, the SB-mRENH1 mRNA, SB-mRENH2 mRNA, and ZFN pair are one or more disclosed in PCT Application Publication WO2023014839A1, which is incorporated by reference herein in its entirety.

In certain embodiments, an LNP of the present disclosure comprises:
(i) an endogenous BCL11a enhancer sequence; and
(ii) a pair of TALENs, each TALEN of the pair comprising a FokI cleavage half-domain and a DNA-binding domain comprising a plurality of TALE repeat units, each repeat unit comprising a hypervariable di-residue region (RVD), wherein the RVDs of the TALE repeats units are shown in a single row of as shown in Table 1, Table 2 or Table 4 of U.S. Pat. No. 11,021,696, and further wherein the pair of TALENs is selected from the group consisting of: 102740 and 102741; 102736 and 102737; 102756 and 102757; 102752 and 102753; 102750 and 102751; 102775 and 102774; 102795 and 102794; 102832 and 102833; 102834 and 102835; 102836 and 102837; 102838 and 102839; 102840 and 102841; 102842 and 102843; 102844 and 102845; 102846 and 102847; 102848 and 102849; 102850 and 102851; 102852 and 102853; 102854 and 102855; 102856 and 102857; 102858 and 102859; 102860 and 102861; 102862 and 102863; 102864 and 102865; 102866 and 102867; 102868 and 102869; 102870 and 102871; 102872 and 102873; 102874 and 102875; 102756 and 102757; 102752 and 102753; 102759 and 102751; 102876 and 102877; 102878 and 102879; 102880 and 102881; 102882 and 102883; 102884 and 102885; 102886 and 102887; 102888 and 102889; 102890 and 102891; 102892 and 102893; 102894 and 102895; 102896 and 102897; 102898 and 102899; 102902 and 102903; 102904 and 102905; 102906 and 102907; 102912 and 102913; 102914 and 102915; 102916 and 102917; 102918 and 102919; 102920 and 102921; or 102922 and 102923, wherein the pair of TALENs cleave and genetically modify the BCL11a enhancer sequence. In certain embodiments, the LNP comprises a polynucleotide encoding any of the TALEN pairs described above. In certain embodiments, an LNP of the present disclosure comprises a DNA binding protein comprising a TALE-effector protein (TALE), wherein the TALE protein comprises a plurality of TALE repeat units, each repeat unit comprising a hypervariable diresidue region (RVD), wherein the RVDs of the TALE repeats units are shown in a single row of Table 1, Table 2 or Table 4 of U.S. Pat. No. 11,021,696. In certain embodiments, the LNP comprises a fusion protein comprising the TALE protein and a wild-type or engineered cleavage domain or cleavage half-domain. In certain embodiments, the LNP comprises a polynucleotide encoding the DNA binding protein described above. In certain embodiments, an LNP of the present disclosure comprises a nuclease, or a polynucleotide encoding said nuclease, capable of making genomic modification selected from an insertion, deletion or combination thereof, within an endogenous BCL11a enhancer sequence. In certain embodiments, the genomic modification is within one or more sequences selected from SEQ ID NO: 1, 2 and 3 of U.S. Pat. No. 11,021,696. In certain embodiments, the genomic modification is at or near one or more sequences selected from SEQ ID NOs: 143 to 184 and 232-251 of U.S. Pat. No. 11,021,696. In certain embodiments, the genomic modification is at or near one or more sequences selected from SEQ ID NOs: 4 to 80 and 362 of U.S. Pat. No. 11,021,696. In certain embodiments, the nuclease comprises at least one ZFN or TALEN. In certain embodiments, the nuclease comprises a zinc finger nuclease, the zinc finger nuclease comprising 4, 5, or 6 zinc finger domains comprising a recognition helix and further wherein the zinc finger proteins comprise the recognition helix regions in the order shown in a single row of Table 3 or Table 6 of U.S. Pat. No. 11,021,696.

In certain embodiments, an LNP of the present disclosure comprises a zinc finger nuclease, or a polynucleotide encoding the same, comprising the amino acid sequence as shown in SEQ ID NO:29 or SEQ ID NO:31 of U.S. Pat. No. 10,563,184. In certain embodiments, the LNP comprises an isolated zinc finger nuclease (ZFN), or a polynucleotide encoding the same, comprising left and right ZFNs, the left ZFN comprising the amino acid sequence as shown in SEQ ID NO:29 and the right ZEN comprising the amino acid sequence as shown in SEQ ID NO:31, both sequences being those disclosed in U.S. Pat. No. 10,563,184. In certain embodiments, the left ZFN is encoded a polynucleotide according to SEQ ID NO:28 and the right ZFN is encoded by a polynucleotide according to SEQ ID NO: 30, both sequences being those disclosed in U.S. Pat. No. 10,563,184. In certain embodiments, the zinc finger nuclease is capable of executing an indel in a BCL11A locus. In certain embodiments, the indel occurs in a BCL11A enhancer sequence. In certain embodiments, the indel comprises inserting an exogenous sequence into an endogenous BCL11A enhancer sequence.

In certain embodiments, an LNP of the present disclosure comprises a zinc finger protein comprising 4, 5 or 6 fingers designated F1 to F4, F1 to F5 or F1 to F6, each finger comprising a recognition helix region that recognizes a target subsite wherein the protein is selected from the group consisting of:

```
(i) a protein comprising the recognition helix
regions as follows:
F1:
  (SEQ ID NO: 7)
STGNLTN;
F2:
(SEQ ID NO: 5)
TSGSLTR;
F3:
(SEQ ID NO: 2)
DQSNLRA;
and
F4:
(SEQ ID NO: 6)
AQCCLFII;
(ii) a protein comprising the recognition helix
regions as follows:
F1:
  (SEQ ID NO: 2)
DQSNLRA;
F2:
  (SEQ ID NO: 3)
RPYTLRL;
F3:
  (SEQ ID NO: 8)
SRGALKT;
F4:
  (SEQ ID NO: 5)
TSGSLTR;
F5:
  (SEQ ID NO: 2)
DQSNLRA;
and
F6:
  (SEQ ID NO: 6)
AQCCLFH;
(iii) a protein comprising the recognition helix
regions as follows:
F1:
  (SEQ ID NO: 2)
DQSNLRA;
F2:
  (SEQ ID NO: 9)
RNFSLTM;
F3:
  (SEQ ID NO: 10)
SNGNLRN
or
(SEQ ID NO: 11)
SSYNLAN;
F4:
(SEQ ID NO: 5)
TSGSLTR;
F5:
  (SEQ ID NO: 2)
DQSNLRA;
and
F6:
  (SEQ ID NO: 6)
AQCCLFH;
(iv) a protein comprising the recognition helix
regions as follows:
F1:
  (SEQ ID NO: 13)
RSDHLTQ;
F2:
  (SEQ ID NO: 14)
QSGHLAR;
F3:
  (SEQ ID NO: 15)
QKGTLGE;
F4:
  (SEQ ID NO: 18)
RHRDLSR;
and
F5:
  (SEQ ID NO: 17)
RRDNLHS;
(v) a protein comprising the recognition helix
regions as follows:
F1:
  (SEQ ID NO: 19)
RNDIIRTT;
F2:
  (SEQ ID NO: 20)
QKAIILIR;
F3:
  (SEQ ID NO: 15)
QKGTLGE;
F4:
  (SEQ ID NO: 25)
LKRTLKR;
and
F5:
(SEQ ID NO: 17)
RRDNLHS;
(vi) a protein comprising the recognition helix
regions as follows:
F1:
  (SEQ ID NO: 13)
RSDHLTQ;
F2:
  (SEQ ID NO: 22)
QRAHLTR;
F3:
  (SEQ ID NO: 15)
QKGTLGE
or
  (SEQ ID NO:24)
QSGTRNH;
F4:
  (SEQ ID NO: 23)
HRNTLVR;
and
F5:
  (SEQ ID NO: 17)
RRDNLHS;
(vii) a protein comprising the recognition helix
regions as follows:
F1:
  (SEQ ID NO: 13)
RSDHLTQ;
F2:
  (SEQ ID NO: 20)
QKAHLIR;
F3:
  (SEQ ID NO: 15)
QKGTLGE
or
(SEQ ID NO: 24)
QSGTRNH;
F4:
(SEQ ID NO: 21)
RGRDLSR;
and
F5:
  (SEQ ID NO: 17)
RRDNLHS;
and
(viii) a protein comprising the recognition helix
regions as follows:
F1:
  (SEQ ID NO: 13)
RSDHLTQ;
F2:
  (SEQ ID NO: 14)
QSGHLAR;
F3:
  (SEQ ID NO: 24)
QSGTRNH;
F4:
  (SEQ ID NO: 16)
QSSDLSR;
and
F5:
  (SEQ ID NO: 17)
RRDNLHS;
```

wherein each of the above SEQ ID NOs correspond with those of U.S. Pat. No. 10,808,020.

In certain embodiments, the LNP comprises a polynucleotide encoding said zinc finger protein. In certain embodiments, the LNP comprises a fusion protein comprising the zinc finger protein and a functional domain, or a polynucleotide encoding the same. In certain embodiments, the functional domain is a transcriptional activation domain, a transcriptional repression domain, or a cleavage domain.

In certain embodiments, an LNP of the present disclosure comprises:
a) a protospacer adjacent motif (PAM) sequence;
b) a trans-activating CRISPR RNA (tracrRNA) sequence; and
c) a single guide RNA selected from the group consisting of SEQ ID NOS: 1-94 disclosed in U.S. Pat. No. 11,572,543.

In certain embodiments, an LNP of the present disclosure further comprises a DNA-targeting endonuclease, or a polynucleotide encoding the same. In certain embodiments, the DNA-targeting endonuclease is a Cas protein. In certain embodiments, the Cas protein is a Cas9.

In certain embodiments, an LNP of the present disclosure comprises a single guide RNA that is:
a. complementary to the plus or minus strand of the human chromosome 2 at location 60725424 to 60/725,688 (+55 functional region);
b. complementary to the plus or minus strand of the human chromosome 2 at location 60722238 to 60/722,466 (+58 functional region); or
c. complementary to the plus or minus strand of the human chromosome 2 at location 60718042 to 60/718,186 (+62 functional region)
wherein the human chromosome 2 is that according to UCSC Genome Browser hg 19 human genome assembly, and
wherein the nucleic acid sequence excludes the entire human chromosome 2 and also excludes the genomic DNA sequence on the human chromosome 2 from location 60,716,189 to 60,728,612.

In certain embodiments, an LNP of the present disclosure comprises an sgRNA targeting a BCL11A genomic region from 0-15 nucleotides upstream of the CTTCCT sequence to 0-15 nucleotides downstream of the CTTCCT sequence in the chromosome 2 in the hematopoietic stem cell is introduced into said hematopoietic stem cells, wherein the BCL11A genomic region is located in a region from chr2:60495236 to chr2:60495271, as described in US Patent Application Publication US20200339979A1. In certain embodiments, the sgRNA construct comprises a nucleotide sequence selected from any one of SEQ ID NOs: 3-25, as disclosed in US Patent Application Publication US20200339979A1. In certain embodiments, the sgRNA comprise a 2′-OMe and or an internucleotide 3′-thio modification. In certain embodiments, the sgRNA comprises a chemical modification comprising a 2′-O-methyl modification of the first one, two and/or three bases at the 5′ end and/or the last base at the 3′ end of a nucleotide sequence. In certain embodiments, the LNP further comprises a gene editing technology. In certain embodiments, the LNP further comprises a zinc finger nuclease-based gene editing technology, a TALEN gene editing technology, or a CRISPR/Cas gene editing technology. In certain embodiments, the LNP further comprises one or more of a basal medium, a growth factor, an antagonist or inhibitor of a progesterone receptor or a glucocorticoid receptor.

In certain embodiments, an LNP of the present disclosure comprises a CRISPR/Cas genome modification system and a BCL11A specific guide RNA, as disclosed in PCT Application Publication No. WO2022240787A1. In certain embodiments, the BCL11A specific guide RNA comprises BCL11A sgRNA 1617 having a sequence of: CTAACAGTTGCTTTTATCAC. In certain embodiments, LNPs comprising the aforementioned payload are suitable for treating subjects having an SNP polymorphism at site rsl 14518452. In certain embodiments, the SNP polymorphism is guanine (G) on the 5′ to 3′ strand.

In certain embodiments, an LNP of the present disclosure comprises a Class 2 Type V CRISPR protein, or a polynucleotide encoding the same, and a first guide ribonucleic acid (gRNA), wherein the gRNA comprises a targeting sequence complementary to a target nucleic acid sequence comprising a polypyrimidine tract-binding protein 1 (BCL11 A) gene. In certain embodiments, the gRNA comprises a targeting sequence complementary to a target nucleic acid sequence selected from the group consisting of a. a BCL11A intron; b. a BCLUA exon; c. a BCL11 A intron-exon junction; d. a BCL11 A regulatory element; and e. an intergenic region. In certain embodiments, the gRNA is a single-molecule gRNA (sgRNA) or a dual-molecule gRNA (dgRNA). In certain embodiments, the targeting sequence of the gRNA comprises a sequence selected from the group consisting of SEQ ID NOS: 272-2100 and 2286-26789 of PCT Application Publication No. WO2022120094A2. In certain embodiments, the targeting sequence has 1, 2, 3, 4, or 5 nucleotides removed from the 3′ end of the sequence. In certain embodiments, the targeting sequence of the gRNA is complementary to a sequence of a BCL11A exon. In certain embodiments, the targeting sequence of the gRNA is complementary to a sequence selected from the group consisting of a BCL11A exon 1 sequence, BCL11A exon 2 sequence, BCL11A exon 3 sequence, BCL11A exon 4 sequence, BCL11A exon 5 sequence, BCL11A exon 6 sequence, BCL11A exon 7 sequence, BCL11A exon 8 sequence, and a BCL11A exon 9 sequence. In certain embodiments, the targeting sequence of the gRNA is complementary to a sequence of a BCL11 A regulatory element. In certain embodiments, the targeting sequence of the gRNA is complementary to a sequence of a promoter of the BCL11 A gene. In certain embodiments, the targeting sequence of the gRNA is complementary to a sequence of an enhancer regulatory element. In certain embodiments, the targeting sequence of the gRNA is complementary to a sequence that comprises a GATA1 erythroid-specific enhancer binding site (GATA1) of the BCL11A gene. In certain embodiments, the targeting sequence of the gRNA is complementary to a sequence that is 5′ to the GATA1 binding site of the BCL11 A gene. In certain embodiments, the targeting sequence of the gRNA comprises a sequence of selected from SEQ ID NOs: 22, 23, 2949, 2948, 15747, or 15748 of PCT Application Publication No. WO2022120094A2 or a sequence having at least 90% or 95% sequence identity thereto. In certain embodiments, the LNP further comprises a second gRNA, wherein the second gRNA has a targeting sequence complementary to a different or overlapping portion of the BCL11 A target nucleic acid compared to the targeting sequence of the gRNA of the first gRNA. In certain embodiments, the targeting sequence of the second gRNA is complementary to a sequence of the target nucleic acid that is 5′ or 3′ to the GATA1 binding site sequence. In certain embodiments, the first and the second gRNA each have a targeting sequence complementary to a sequence within the promoter of the BCL11 A gene. In certain embodiments, the first or second gRNA has a scaffold comprising a sequence selected from the group consisting of SEQ ID NOS: 2238-2285, 26794-26839 and 27219-27265 of PCT Application Publication No. WO2022120094A2, or a sequence having at least about 50%, at least about 60%, at least about 70%, at least about 80%, at least about 90%, at least about 95%, at least about 96%, at least about 97%, at least about 98%, at least about 99% sequence identity thereto. In certain embodiments, the first or second gRNA has a scaffold comprising or consisting of a sequence selected from the group consisting of SEQ ID NOs: 2238-2285, 26794-26839 and 27219-27265 of PCT Application Publication No. WO2022120094A2. In certain embodiments, the Class 2 Type V CRISPR protein is a CasX variant protein comprising a sequence selected from the group consisting of SEQ ID NOS: 59, 72-99, 101-148, and 26908-27154 of PCT Application Publication No. WO2022120094A2, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto. In certain embodiments, the CasX variant protein consists of a sequence selected from the group consisting of SEQ ID NOS: 126, 27043, 27046, 27050 of PCT Application Publication No. WO2022120094A2. In certain embodiments, the CasX variant protein comprises at least one modification relative to a reference CasX protein having a sequence selected from SEQ ID NOS: 1-3 of PCT Application Publication No. WO2022120094A2. In certain embodiments, the at least one modification comprises at least one amino acid substitution, deletion, or substitution in a domain of the CasX variant protein relative to the reference CasX protein.

In certain embodiments, an LNP of the present disclosure comprises one or more single-molecule guide RNA (sgRNAs), wherein at least one of the one or more sgRNAs comprises (a) the nucleic acid sequence of any one of SEQ ID NOs: 13-18 of U.S. Pat. No. 11,268,077 or (b) a spacer sequence comprising an RNA sequence corresponding to any one of SEQ ID NOs: 6-8 of U.S. Pat. No. 11,268,077 or the nucleic acid sequence of any one of SEQ ID NOs: 19-21 of U.S. Pat. No. 11,268,077. In certain embodiments, the LNP further comprises one or more DNA endonucleases, or polynucleotides encoding the same. In certain embodiments, the one or more DNA endonucleases comprises a Cas9 endonuclease, or polynucleotides encoding the same. In certain embodiments, the one or more DNA endonucleases each comprise, at the N-terminus, the C-terminus, or both the N-terminus and C-terminus, one or more nuclear localization signals (NLSs), optionally wherein the one or more DNA endonucleases each comprise two NLSs, one NLS located at the N-terminus and the second NLS located at the C-terminus, further optionally wherein the one or more NLSs is a SV40 NLS. In certain embodiments, the sgRNA comprises the nucleic acid sequence of any one of SEQ ID NO: 13-18 of U.S. Pat. No. 11,268,077 and the DNA endonuclease is a S. pyogenes Cas9 comprising a N-terminus SV40 NLS and a C-terminus SV40 NLS.

In certain embodiments, an LNP of the present disclosure comprises one or more deoxyribonucleic acid (DNA) endonucleases capable of effecting two or more single-strand breaks (SSB) or double-strand breaks (DSB), a first SSB or DSB within or near a human beta globin locus on chromosome 11, and a second SSB or DSB within or near one or more of: a BCL11A gene on chromosome 2, and a DNA sequence that encodes a transcriptional control region of the BCL11A gene on chromosome 2. In certain embodiments, the first SSB or DSB results in one or more of: a permanent deletion or inversion within or near the human beta globin locus on chromosome 11, and a mutation within or near the human beta globin locus on chromosome 11. In certain embodiments, the second SSB or DSB results in one or more of: a permanent deletion or inversion within or near the BCL11A gene on chromosome 2, a mutation within or near the BCL11A gene on chromosome 2, a permanent deletion or inversion within or near the DNA sequence that encodes a transcriptional control region of the BCL11A gene on chromosome 2, and a mutation within or near the DNA sequence that encodes a transcriptional control region of the BCL11A gene on chromosome 2. In certain embodiments, the two or more permanent deletions, inversions, or mutations result in an increased expression of γ-globin and an increase of fetal hemoglobin (HbF) in the human cell. Such endonucleases are generally described in US Patent Application Publication US20210180091A1, which is incorporated by reference herein in its entirety. In certain embodiments, the LNP further comprises one or more guide RNA (gRNA). In certain embodiments, the one or more gRNA sequences are selected from those provided in US Patent Application Publication US20210180091A1 Table 2, optionally wherein the one or more gRNAs comprise the sequence of SPY101 in combination with the sequence of any one or more of HPFH5-1, HPFH5-D, HPFH5-T5, HPFH5-T7, Kenya-K5, or Kenya-K1; or optionally wherein the one or more gRNAs comprise the sequence of 1450 in combination with the sequence of any one or more of HPFH5-1, HPFH5-D, HPFH5-T5, HPFH5-T7, Kenya-K5, or Kenya-K1; or optionally wherein the one or more gRNAs comprise the sequence of SPY101 in combination with the sequence of SD2; or optionally wherein the one or more gRNAs comprise the sequence of 1450 in combination with the sequence of SD2; or optionally wherein the one or more gRNAs comprise the sequence of HPFH5-1, HPFH5-D, HPFH5-T5, HPFH5-T7, Kenya-K5, or Kenya-K1 in combination with the sequence of any one or more of HPFH5-1, HPFH5-D, HPFH5-T5, HPFH5-T7, Kenya-K5, or Kenya-K1.

In certain embodiments, an LNP of the present disclosure comprises a guide RNA (gRNA) molecule comprising, from 5′ to 3′, [targeting domain]-SEQ ID NO: 195, wherein the targeting domain comprises SEQ ID NO: 67, wherein the referenced sequences are those disclosed in U.S. Pat. No. 11,466,271. In certain embodiments, said SEQ ID NO: 195 is disposed immediately 3′ to said targeting domain. In certain embodiments, an LNP of the present disclosure comprises a guide RNA (gRNA) molecule comprising, from 5′ to 3′, [targeting domain]-SEQ ID NO: 231, wherein the targeting domain comprises SEQ ID NO: 67, wherein the referenced sequences are those disclosed in U.S. Pat. No. 11,466,271. In certain embodiments, said SEQ ID NO: 231 is disposed immediately 3′ to said targeting domain. In certain embodiments, the gRNA further comprises, at the 3′ end, 1, 2, 3, 4, 5, 6 or 7 uracil (U) nucleotides. In certain embodiments, the LNP further comprises a CRISPR system capable of utilizing the gRNA to form an indel at or near a target sequence complementary to the targeting domain of the gRNA. In certain embodiments, the indel is formed in a region selected from the IIBG1 promoter region, the HIBG2 promoter region. In certain embodiments the CRISPR system comprises a Cas9. In certain embodiments, the gRNA comprises a sequence selected from SEQ ID NOs: 174, 175 and 176 of U.S. Pat. No. 11,466,271. In certain embodiments, the Cas9 molecule comprises a sequence selected from SEQ ID NOs: 233, 234, 235, 236, 237, 238, 239, 240, 241, 242, 243, and 244.

In certain embodiments, the LNP comprises a gRNA comprising a tracr and crRNA, wherein the crRNA comprises a targeting domain that:
a) is complementary with a target sequence within the genomic nucleic acid sequence at Chrl 1:5,250,094-5,250,237, − strand, hg38;
b) is complementary with a target sequence within the genomic nucleic acid sequence at Chrl 1:5,255,022-5,255,164, − strand, hg38;
c) is complementary with a target sequence of a non-deletional HFPH region (e.g., a human nondeletional HPFH region);
d) is complementary with a target sequence within the genomic nucleic acid sequence at Chrl 1:5,249,833 to Chrl 1:5,250,237, − strand, hg38;
e) is complementary with a target sequence within the genomic nucleic acid sequence at Chrl 1:5,254,738 to Chrl 1:5,255, 164, − strand, hg38;
f) is complementary with a target sequence within the genomic nucleic acid sequence at Chrl 1:5, 249,833-5,249,927, − strand, hg38;
g) is complementary with a target sequence within the genomic nucleic acid sequence at Chrl 1:5, 254,738-5,254,851, − strand, hg38;
h) is complementary with a target sequence within the genomic nucleic acid sequence at Chrl 1:5,250, 139-5,250,237, − strand, hg38; or
i) combinations thereof.

In certain embodiments, the gRNA comprises a sequence of any one of SEQ ID NOs. 1-72 of U.S. Pat. No. 11,466,271, or a fragment thereof.

In certain embodiments, an LNP of the present disclosure comprises a gene editing system capable of targeting the HBG1 promoter (Chrl 1:5,249,833-5,250,237 according to hg38) and/or HBG2 promoter (Chrl 1:5,254,738-5,255,164 according to hg38). In certain embodiments, the gene editing system targets a targeting locus selected from the HPFH region, the AAVS1 locus, the BCL11a gene, and the BCL11a enhancer region. In certain embodiments, the target locus is: a) the +55 region of the BCL11a enhancer (Chr2: 60497676-60498941 according to hg38); b) the +58 region of the BCL11a enhancer (Chr2: 60494251-60495546 according to hg38); or c) the +62 region of the BCL11a enhancer (Chr2: 60490409-60491734 according to hg38). In certain embodiments, the gene editing system comprises a) a zinc finger nuclease (ZFN) system; b) a TALEN system; c) a meganuclease system; or d) a CRISPR system. In certain embodiments, the gene editing system comprises a CRISPR system comprising a gRNA molecule comprising a targeting domain complementary to any one of SEQ ID NO: 1 to 161, and 197 of PCT Publication WO2017/077394, or any one of SEQ ID NO: 1 to 135 of PCT Publication WO2016/182917. In certain embodiments, the gene editing system comprises a ZFN system comprising a targeting domain complementary to any one of SEQ ID NO: 63-80 and 232-251 of PCT Publication WO2015/073683. In certain embodiments, the gene editing system comprises a TALEN system comprising a targeting domain complementary to any one of SEQ ID NO: 7-11, 16-62, and 143-184 of PCT Publication WO2015/073683. In certain embodiments, the target sequence is a target sequence identified in Table 6 of US Application Publication US20200140896A1, and the gene editing system is a CRISPPv gene editing system comprising a gRNA molecule comprising a targeting domain listed in Table 6 of US Application Publication US20200140896A1. All patent application publication references in the above paragraph are incorporated by reference herein in their entirety.

In certain embodiments, an LNP of the present disclosure comprises a gRNA molecule comprising a tracr and crRNA, wherein the crRNA comprises a targeting domain that is complementary with a target sequence of a BCL11A gene (e.g., a human BCL11a gene), a BCL11a enhancer (e.g., a human BCL11a enhancer), or a HFPH region (e.g., a human HPFH region). In certain embodiments, the target sequence is of the BCL11A gene, and the targeting domain comprises, e.g., consists of, any one of SEQ ID NO: 1 to SEQ ID NO: 85 or SEQ ID NO: 400 to SEQ ID NO: 1231 of US Application Publication US20190010495A1. In certain embodiments, the target sequence is of a BCL11a enhancer, and the targeting domain comprises, e.g., consists of, any one of SEQ ID NO: 1232 to SEQ ID NO: 1499 of US Application Publication US20190010495A1. In certain embodiments, the target sequence is of a BCL11a enhancer, and the targeting domain comprises, e.g., consists of, any one of SEQ ID NO: 182 to SEQ ID NO: 277 or SEQ ID NO: 334 to SEQ ID NO: 341 of US Application Publication US20190010495A1. In certain embodiments, the targeting domain comprises, e.g., consists of, any one of SEQ ID NO: 341, SEQ ID NO: 246, SEQ ID NO: 248, SEQ ID NO: 247, SEQ ID NO: 245, SEQ ID NO: 249, SEQ ID NO: 244, SEQ ID NO: 199, SEQ ID NO: 251, SEQ ID NO: 250, SEQ ID NO: 334, SEQ ID NO: 185, SEQ ID NO: 186, SEQ ID NO: 336, or SEQ ID NO: 337 of US Application Publication US20190010495A1. In certain embodiments, the targeting domain comprises, e.g., consists of, any one of SEQ ID NO: 247, SEQ ID NO: 248, SEQ ID NO: 335, SEQ ID NO: 336, SEQ ID NO: 337, or SEQ ID NO: 338 of US Application Publication US20190010495A1. In certain embodiments, the targeting domain comprises, e.g., consists of, any one of SEQ ID NO: 248 or SEQ ID NO: 338 of US Application Publication US20190010495A1. In certain embodiments, the target sequence is a BCL11a enhancer, and the targeting domain comprises, e.g., consists of, any one of SEQ ID NO: 278 to SEQ ID NO: 333.

In certain embodiments, the targeting domain comprises, e.g., consists of, any one of SEQ ID NO: 318, SEQ ID NO: 312, SEQ ID NO: 313, SEQ ID NO: 294, SEQ ID NO: 310, SEQ ID NO: 319, SEQ ID NO: 298, SEQ ID NO: 322, SEQ ID NO: 311, SEQ ID NO: 315, SEQ ID NO: 290, SEQ ID NO: 317, SEQ ID NO: 309, SEQ ID NO: 289, or SEQ ID NO: 281 of US Application Publication US20190010495A1. In certain embodiments, the targeting domain comprises, e.g., consists of, SEQ ID NO: 318 of US Application Publication US20190010495A1. In certain embodiments, the target sequence is of a BCL11a enhancer, and the targeting domain comprises, e.g., consists of, any one of SEQ ID NO: 1596 to SEQ ID NO: 1691 of US Application Publication US20190010495A1. In certain embodiments, the targeting domain comprises, e.g., consists of, any one of SEQ ID NO: 1683, SEQ ID NO: 1638, SEQ ID NO: 1647, SEQ ID NO: 1609, SEQ ID NO: 1621, SEQ ID NO: 1617, SEQ ID NO: 1654, SEQ ID NO: 1631, SEQ ID NO: 1620, SEQ ID NO: 1637, SEQ ID NO: 1612, SEQ ID NO: 1656, SEQ ID NO: 1619, SEQ ID NO: 1675, SEQ ID NO: 1645, SEQ ID NO: 1598, SEQ ID NO: 1599, SEQ ID NO: 1663, SEQ ID NO: 1677, or SEQ ID NO: 1626 of US Application Publication US20190010495A1. In certain embodiments, the target sequence is of a HFPH region (e.g., a French HPFH region), and the targeting domain comprises, e.g., consists of, any one of SEQ ID NO: 86 to SEQ ID NO: 181, SEQ ID NO: 1500 to SEQ ID NO: 1595, or SEQ ID NO: 1692 to SEQ ID NO: 1761 of US Application Publication US20190010495A1. In certain embodiments, the targeting domain comprises, e.g., consists of, any one of SEQ ID NO: 100, SEQ ID NO: 165, SEQ ID NO: 113, SEQ ID NO: 99, SEQ ID NO: 112, SEQ ID NO: 98, SEQ ID NO: 1580, SEQ ID NO: 106, SEQ ID NO: 1503, SEQ ID NO: 1589, SEQ ID NO: 160, SEQ ID NO: 1537, SEQ ID NO: 159, SEQ ID NO: 101, SEQ ID NO: 162, SEQ ID NO: 104, SEQ ID NO: 138, SEQ ID NO: 1536, SEQ ID NO: 1539, or SEQ ID NO: 1585 of US Application Publication US20190010495A1. In certain embodiments, the targeting domain comprises, e.g., consists of, any one of SEQ ID NO: 98, SEQ ID NO: 100, SEQ ID NO: 1505, SEQ ID NO: 1589, SEQ ID NO: 1700, or SEQ ID NO: 1750 of US Application Publication US20190010495A1. In certain embodiments, the targeting domain comprises, e.g., consists of, any one of SEQ ID NO: 100, SEQ ID NO: 165, or SEQ ID NO: 113 of US Application Publication US20190010495A1. In certain embodiments, the gRNA comprises or consists of any guide RNA sequence, or any portion thereof, disclosed in US Application Publication US20190010495A1, which is incorporated by reference herein in its entirety.

In certain embodiments, an LNP of the present disclosure comprises an RNP complex comprising a first gRNA and a Cpf1 RNA-guided nuclease or a modified Cpf1 RNA-guided nuclease, wherein RNP complex is capable of generating indels in a population of unmodified CD34+ or hematopoietic stem cells. In certain embodiments, the first gRNA comprises a 5′ end and a 3′ end, a DNA extension at the 5′ end, and a 2′-O-methyl-3′-phosphorothioate modification at the 3′ end. In certain embodiments, the DNA extension comprises a sequence set forth in SEQ ID NOs: 1235-1250 of PCT Publication WO2021119040A1. In certain embodiments, the first gRNA comprises a targeting domain comprising SEQ ID NO:1254 of PCT Publication WO2021119040A1. In certain embodiments, the gRNA comprises SEQ ID NO:1051 of PCT Publication WO2021119040A1. In certain embodiments, the modified Cpf1 comprises SEQ ID NO: 1097 of PCT Publication WO2021119040A1.

In certain embodiments, an LNP of the present disclosure comprises a gRNA molecule comprising a targeting domain that is complementary to a target domain from a gene expressed in a eukaryotic cell, wherein the gRNA molecule comprises a 3′ polyA tail comprising fewer than 30 adenine nucleotides. In certain embodiments, the polyA tail comprises fewer than 25 adenine nucleotides. In certain embodiments, the gRNA molecule lacks a 5′ triphosphate group. In certain embodiments, the gRNA molecule includes a 5′ cap. In certain embodiments, the gRNA molecule comprises a targeting domain and the 5′ end of the targeting domain includes a 5′ cap. In certain embodiments, the gRNA molecule contains one or more nucleotides that stabilize the gRNA molecule against nuclease degradation. In certain embodiments, the gRNA molecule contains one or more modified uridines. In certain embodiments, the gRNA molecule contains one or more modified adenosines. In certain embodiments, the gRNA molecule contains one or more modified cytidines. In certain embodiments, the gRNA molecule contains one or more modified guanosines. In certain embodiments, the phosphate backbone of the gRNA is modified. In certain embodiments, the phosphate backbone of the gRNA is modified with a phosphorothioate group. In certain embodiments, the gRNA comprises or consists of any guide RNA sequence, or any portion thereof, disclosed in US Application Publication US20230002760A1, which is incorporated by reference herein in its entirety. In certain embodiments, the LNP further comprises a CRISPR Cas9, or a polynucleotide encoding the same, disclosed in US Application Publication US20230002760A1, which is incorporated by reference herein in its entirety.

In certain embodiments, an LNP of the present disclosure comprises a first gRNA, a first enzymatically active Cas9 (eaCas9) (or a polynucleotide encoding the same), a second gRNA and a second eaCas9 (or a polynucleotide encoding the same). In certain embodiments, the first gRNA molecule and the first eaCas9 molecule associate with a target gene and generate a first single strand cleavage event on a first strand of the target gene, and the second gRNA molecule and the second eaCas9 molecule associate with the target gene and generate a second single strand cleavage event on a second strand of the target gene, thereby forming a double strand break having a first overhang and a second overhang. In certain embodiments, the first and second overhang are repaired by gene conversion using an endogenous homologous region. In certain embodiments, the first eaCas9 and/or the second eaCas9 are a nickase, optionally the same nickase or a different nickase. In certain embodiments, the first gRNA, the first eaCas9 (or a polynucleotide encoding the same), the second gRNA and the second eaCas9 (or a polynucleotide encoding the same) are capable of correcting a mutation, such as but not limited to a mutation in an endogenous hemoglobin beta (HBB) target gene. In certain embodiments, the first gRNA molecule is a gRNA molecule comprising any one of SEQ ID NOs: 387-485, 6803-6871, or 16010-16256, and wherein the second gRNA molecule is a gRNA molecule comprising any one of SEQ ID NOs: 387-485, 6803-6871, or 16010-16256, wherein the sequences are those disclosed in US Patent Application Publication US20230018543A1, which is incorporated by reference herein in its entirety.

In certain embodiments, an LNP of the present disclosure comprises a gene editing system described and disclosed in US Patent Application Publication US20210155927A1. In certain embodiments, the LNP comprises:
a) a ribonucleoprotein (RNP) complex comprising:
i) a class 2 CRISPR/Cas effector polypeptide, or a nucleic acid comprising a nucleotide sequence encoding the class 2 CRISPR/Cas effector polypeptide; and
ii) a guide RNA; and
b) a donor DNA template comprising a nucleotide sequence that provides for correction of the SCD-associated SNP in the globin gene.

In certain embodiments, the class 2 CRISPR/Cas effector polypeptide is selected from a type II, type V, or type VI CRISPR/Cas effector polypeptide, a Cpf1 protein, a C2c1 protein, a C2c3 protein, a C2c2 protein, a Cas12 enzyme, or a Cas13 enzyme or nucleic acid encoding any of the aforementioned proteins. In certain embodiments, the CRISPR/Cas guide RNA is a Cas9 guide RNA.

In certain embodiments, an LNP of the present disclosure comprises a gene editing system described and disclosed in US Patent Application Publication US20200330609A1, which is incorporated by reference herein, in its entirety. In certain embodiments, the LNP comprises: a) a S. pyogenes Cas9 endonuclease, or a nucleotide sequence encoding the same; and b) one or more gRNAs comprising a single molecule gRNA (sgRNA) comprising the nucleic acid sequence of SEQ ID NO: 71,959 of US Application Publication US20200330609A1.

In certain embodiments, the LNP comprises an sgRNA comprising the nucleic acid sequence of SEQ ID NO: 71,959 of US Application Publication US20200330609A1, wherein the sgRNA comprises three 2′O-methyl-phosphorothioate residues at each of its 5′ and 3′ ends. In certain embodiments, the LNP comprises an sgRNA comprising the nucleic acid sequence of SEQ ID NO: 71,959 of US Application Publication US20200330609A1, wherein bases 1-3 and 97-99 are modified 2′-O-methyl phosphorothioate nucleotides.

In certain embodiments, an LNP of the present disclosure comprises a gene editing system described and disclosed in US Patent Application Publication US20200384033A1, which is incorporated by reference herein, in its entirety. In certain embodiments, the LNP comprises a Cas9 endonuclease and a guide RNA (gRNA) that targets the +58 DHS of a human BCL11A gene wherein the gRNA comprises a nucleotide sequence of SEQ ID NO:1 or SEQ ID NO:2 of US Patent Application Publication US20200384033A1. In certain embodiments, the gRNA comprises three 2′-O-methyl-phosphorothioate residues at or near each of its 5′ and 3′ ends.

In certain embodiments, an LNP of the present disclosure comprises a gene editing system described and disclosed in PCT Publication WO2017191503A1, which is incorporated by reference herein, in its entirety. In certain embodiments, the LNP comprises one or more deoxyribonucleic acid (DNA) endonucleases capable of affecting one or more single-strand breaks (SSBs) or double-strand breaks (DSBs) within or near the human beta globin locus on chromosome 11 that results in at least one permanent deletion, at least one mutation, or at least one permanent deletion and at least one mutation within or near the human beta globin locus and results in an increase of fetal hemoglobin (HbF) in the human cell. In certain embodiments, the permanent deletion and/or mutation within or near the human beta globin locus on chromosome is a 4.9 kb deletion upstream of the HBG1 gene, a 13-base pair deletion upstream of the HBG1 gene or both of the aforementioned. In certain embodiments, the deletion includes the HBG2 gene.

In certain embodiments, an LNP of the present disclosure comprises a gene editing system described and disclosed in PCT Publication WO2021144692A1, which is incorporated by reference herein, in its entirety. In certain embodiments, the LNP comprises at least one CRISPR system or nucleic acid encoding at least one CRISPR system to generate at least one double stranded break at a target locus. In certain embodiments, the Cas9 nuclease or Cpf1 nuclease is chosen from S. pyogenes Cas9, S. aureus Cas9, N. meningitides Cas9, S. thermophilus CRISPR1 Cas9, S. thermophilus CRISPR 3 Cas9, T. denticola Cas9, L. bacterium ND2006 Cpf1, Acidaminococcus sp. BV3L6 Cpf1, or the variant thereof has at least 90% sequence identity to said nuclease. In certain embodiments, the CRISPR nuclease further comprises at least one nuclear localization signal (NLS) at or within 50 amino acids of the amino-terminus and/or the carboxy-terminus. In certain embodiments, the donor template comprises a donor sequence that is flanked by sequence homologous to the target locus. In certain embodiments, the donor sequence comprises at least one exon and/or intron of a target gene and wherein the target locus is located within the target gene. In certain embodiments, the target locus is a RAGI target locus, an IL7R target locus, or a HBB target locus.

In certain embodiments, an LNP of the present disclosure comprises a gene editing system described and disclosed in PCT Publication WO2023248147A1, which is incorporated by reference herein, in its entirety. In certain embodiments, the LNP comprises at least one guide RNA (gRNA) that targets a genomic region of interest or a nucleic acid encoding the at least one gRNA, and a nucleic acid encoding a RNA-guided endonuclease. In certain embodiments, the at least one gRNA is a single-guide RNA (sgRNA). In certain embodiments, the at least one gRNA targets the erythroid specific enhancer of BCL11 A gene. In certain embodiments, the Cas9 endonuclease is S. pyogenes Cas9 (SpCas9), S. aureus Cas9 (SaCas9), S. lugdunensis Cas9 (SluCas9), N meningitidis Cas9, S. thermophilus Cas9, S. thermophilus 3 Cas9, T. denticola Cas9, C. jejuni Cas9 (CjCas9), or a variant thereof.

### B. Nucleic Acid Payloads

In various embodiments, the LNP compositions described herein can be used to deliver a nucleic acid or polynucleotide payload, e.g., a linear or circular mRNA.

In some embodiments, a LNP is capable of delivering a polynucleotide to a target cell, tissue, or organ. A polynucleotide, in its broadest sense of the term, includes any compound and/or substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. RNAs useful in the compositions and methods described herein can be selected from the group consisting of but are not limited to, shortimers, antagomirs, antisense, ribozymes, short interfering RNA (siRNA), asymmetrical interfering RNA (aiRNA), microRNA (miRNA), Dicer substrate RNA (dsRNA), short hairpin RNA (shRNA), transfer RNA (tRNA), messenger RNA (mRNA), and mixtures thereof. In some embodiments, a polynucleotide is mRNA. In some embodiments, a polynucleotide is circular RNA. In some embodiments, a polynucleotide encodes a protein, e.g., a nucleobase editing enzyme. A polynucleotide may encode any polypeptide of interest, including any naturally or non-naturally occurring or otherwise modified polypeptide. A polypeptide may be of any size and may have any secondary structure or activity. In some embodiments, a polypeptide encoded by an mRNA may have a therapeutic effect when expressed in a cell.

In other embodiments, a polynucleotide is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In some embodiments, a polynucleotide is an shRNA or a vector or plasmid encoding the same. An shRNA may be produced inside a target cell upon delivery of an appropriate construct to the nucleus. Constructs and mechanisms relating to shRNA are well known in the relevant arts.

A polynucleotide may include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, a polynucleotide further includes a poly-A region or a Kozak sequence (e.g., in the 5′-UTR). In some cases, polynucleotides may contain one or more intronic nucleotide sequences capable of being excised from the polynucleotide. In some embodiments, a polynucleotide (e.g., an mRNA) may include a 5′cap structure, a chain terminating nucleotide, a stem loop, a polyA sequence, and/or a polyadenylation signal.

In various embodiments, the nucleic acid payloads may contain one or more modifications. Such modifications include various chemical and/or structural modifications. For example, in the case of RNA, the RNA may comprise one or more modifications, including chemical modifications (e.g., ribonucleotide analogs, alternative phosphate chain linkers), sequence modification (e.g., relative to a wild type sequence), and/or structural modification (e.g., secondary-folded structures, such as, but not limited to, stem-loops, hairpins, and G-quadruplexes, and tertiary structural elements, such as, but not limited to, helical duplexes and triple-stranded structures). To date, hundreds of different RNA modifications have been characterized. Among them, several RNA modifications, including N⁶-methyladenosine (m⁶A), N⁶,2′-O-dimethyladenosine (m⁶Am), 8-oxo-7,8-dihydroguanosine (8-oxoG), pseudouridine (Ψ), 5-methylcytidine (m⁵C), and N-acetylcytidine (ac⁴C), have been shown to regulate mRNA stability, consequently affecting diverse cellular and biological processes. Any known modification to RNA or DNA is contemplated herein.

In some embodiments, a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl pseudouridine or 1-ethyl-pseudouridine), and/or a 5-substituted cytidine (e.g., 5-methyl-cytidine). In some embodiments, a polynucleotide contains only naturally occurring nucleosides.

Nucleic acid modifications are well known in the art and are further discussed in the following references: (1) Crooke S T, Witztum J L, Bennett C F, Baker B F. RNA-Targeted Therapeutics. Cell Metab. 2018 Apr. 3; 27(4):714-739. doi: 10.1016/j.cmet.2018.03.004. Erratum in: Cell Metab. 2019 Feb. 5; 29(2):501. PMID: 29617640; (2) JP, Wen W, Zhang F, Oberg K C, Zhang L, Cheng T, Zhang X B. Dynamics and competition of CRISPR-Cas9 ribonucleoproteins and AAV donor-mediated NHEJ, MMEJ and HDR editing. Nucleic Acids Res. 2021 Jan. 25; 49(2):969-985. doi: 10.1093/nar/gkaa1251. PMID: 33398341; PMCID: PMC7826255; (3) Pradeep S P, Malik S, Slack F J, Bahal R. Unlocking the potential of chemically modified peptide nucleic acids for RNA-based therapeutics. RNA. 2023 April; 29(4):434-445. doi: 10.1261/rna.079498.122. Epub 2023 Jan. 18. PMID: 36653113; PMCID: PMC10019372; (4) Haruehanroengra P, Zheng Y Y, Zhou Y, Huang Y, Sheng J. RNA modifications and cancer. RNA Biol. 2020 November; 17(11):1560-1575. doi: 10.1080/15476286.2020.1722449. Epub 2020 Feb. 7. PMID: 31994439; PMCID: PMC7567502; (5) Heidenreich O, Pieken W, Eckstein F. Chemically modified RNA: approaches and applications. FASEB J. 1993 January; 7(1):90-6. doi: 10.1096/fasebj.7.1.7678566. PMID: 7678566; (6) Zhang H Y, Du Q, Wahlestedt C, Liang Z. RNA Interference with chemically modified siRNA. Curr Top Med Chem. 2006; 6(9):893-900. doi: 10.2174/156802606777303676. PMID: 16787282; (7) Jin G, Xu M, Zou M, Duan S. The Processing, Gene Regulation, Biological Functions, and Clinical Relevance of N4-Acetylcytidine on RNA: A Systematic Review. Mol Ther Nucleic Acids. 2020 Jun. 5; 20:13-24. doi: 10.1016/j.omto.2020.01.037. Epub 2020 Feb. 8. PMID: 32171170; PMCID: PMC7068197; (8) Gao M, Zhang Q, Feng X H, Liu J. Synthetic modified messenger RNA for therapeutic applications. Acta Biomater. 2021 Sep. 1; 131:1-15. doi: 10.1016/j.actbio.2021.06.020. Epub 2021 Jun. 13. PMID: 34133982; PMCID: PMC8198544; (9) Filippova J A, Somenov D V, Juravlov E S, Komissarov A B, Richter V A, Stepanov G A. Modern Approaches for Identification of Modified Nucleotides in RNA. Biochemistry (Mosc). 2017 November; 82(11):1217-1233. doi: 10.1134/80006297917110013. PMID: 29223150; (10) Rothlisberger P, Berk C, Hall J. RNA Chemistry for RNA Biology. Chimia (Aarau). 2019 May 29; 73(6):368-373. doi: 10.2533/chimia.2019.368. PMID: 31118118; and (11) Elkhalifa D, Rayan M, Negmeldin A T, Elhissi A, Khalil A. Chemically modified mRNA beyond COVID-19: Potential preventive and therapeutic applications for targeting chronic diseases. Biomed Pharmacother. 2022 January; 145:112385. doi: 10.1016/j.biopha.2021.112385. Epub 2021 Oct. 28. PMID: 34915673; PMCID: PMC8552589; (12) Boo S H, Kim Y K. The emerging role of RNA modifications in the regulation of mRNA stability. Exp Mol Med. 2020 March; 52(3):400-408. doi: 10.1038/s12276-020-0407-z. Epub 2020 Mar. 24. PMID: 32210357; PMCID: PMC7156397; (13) Varshney D, Spiegel J. Zyner K, Tannahill D, Balasubramanian S. The regulation and functions of DNA and RNA G-quadruplexes. Nat Rev Mol Cell Biol. 2020 August; 21(8):459-474. doi: 10.1038/s41580-020-0236-x. Epub 2020 Apr. 20. PMID: 32313204; PMCID: PMC7115845; each of which are incorporated herein by reference in their entireties. In some cases, a polynucleotide is greater than 30 nucleotides in length. In another embodiment, the poly nucleotide molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 50 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

In some embodiments, a polynucleotide molecule, formula, composition or method associated therewith comprises one or more polynucleotides comprising features as described in WO2002/098443, WO2003/051401, WO2008/052770, WO2009/127230, WO2006/122828, WO2008/083949, WO2010/088927, WO2010/037539, WO2004/004743, WO2005/016376, WO2006/024518, WO2007/095976, WO2008/014979, WO2008/077592, WO2009/030481, WO2009/095226, WO2011/069586, WO2011/026641, WO2011/144358, WO2012/019780, WO2012/013326, WO2012/089338, WO2012/113513, WO2012/116811, WO2012/116810, WO2013/113502, WO2013/113501, WO2013/113736, WO2013/143698, WO2013/143699, WO2013/143700, WO2013/120626, WO2013/120627, WO2013/120628, WO2013/120629, WO2013/174409, WO2014/127917, WO2015/024669, WO2015/024668, WO2015/024667, WO2015/024665, WO2015/024666, WO2015/024664, WO2015/101415, WO2015/101414, WO2015/024667, WO2015/062738, WO2015/101416, all of which are incorporated by reference herein.

In some embodiments, a polynucleotide comprises one or more microRNA binding sites. In some embodiments, a microRNA binding site is recognized by a microRNA in a non-target organ. In some embodiments, a microRNA binding site is recognized by a microRNA in the liver. In some embodiments, a microRNA binding site is recognized by a microRNA in hepatic cells.

In certain embodiments, an RNA of the present disclosure comprises one or more phosphonate modifications selected from a phosphorothioate linkage (PS), phosphorodithioate linkage (PS2), methylphosphonate linkage (MP), methoxypropylphosphonate linkage (MOP), 5′-(E)-vinylphosphonate linkage (5′-(E)-VP), 5′-Methyl Phosphonate linkage (5′-MP), (S)-5′-C-methyl with phosphate linkage, 5′-phosphorothioate linkage (5′-PS), and a peptide nucleic acid linkage (PNA). In certain embodiments, an RNA of the present disclosure comprises one or more ribose modifications selected from a 2′-O-methyl(2′-OMe), 2′-O-methoxyethyl (2′-O-MOE), 2′-deoxy-2′-fluoro (2′-F), 2′-arabino-fluoro (2′-Ara-F), 2′-O-benzyl, 2′-O-methyl-4-pyridine (2′-O-CH₂Py(4)), Locked nucleic acid (LNA), (S)-cET-BNA, tricyclo-DNA (tcDNA), PMO, Unlocked Nucleic Acid (UNA) and glycol nucleic acid (GNA). In certain embodiments, the RNA comprises a Locked Nucleic Acid (LNA) comprising a methyl bridge, an ethyl bridge, a propyl bridge, a butyl bridge or an optionally substituted variant of any of the aforementioned. In certain embodiments, an RNA of the present disclosure comprises one or more modified bases selected from a pseudouridine (ψ), 2′thiouridine (s2U), N6′-methyladenosine (m⁶A), 5′methylcytidine (m⁵C), 5′fluoro2′-deoxyuridine, N-ethylpiperidine 7′-EAA triazole modified adenine, N-ethylpiperidine 6′triazole modified adenine, 6′pheynlpyrrolo-cytosine (PhpC), 2′,4′-difluorotoluyl ribonucleoside (rF), and 5′-nitroindole.

C. Linear mRNA Payloads

In various embodiments, the LNP-based nucleobase editing systems, RNA therapeutics and pharmaceutical compositions thereof described herein can be used to deliver an RNA payload that is a linear mRNA molecule.

In various embodiments, the LNP-based pharmaceutical compositions described herein, e.g., LNP-based gene editing systems, may include one or more linear mRNA molecules or linear mRNA payloads. In various embodiments, the mRNA payloads may encode one or more components of the herein described gene editing systems. For example, an mRNA payload may encode an amino acid sequence-programmable DNA binding domain (e.g., TALENS and zinc finger-binding domains) or a nucleic acid sequence-programmable DNA binding domain (e.g., CRISPR Cas9, CRISPR Cas12a, CRISPR Cas12f, CRISPR Cas13a, CRISPR Cas13b, or TnpB).

mRNA payloads may also encode, depending upon the nature of the gene editing system, one or more effector domains that provide various functionalities that facilitate changes in nucleotide sequence and/or gene expression, such as, but not limited to, single-strand DNA binding proteins, nucleases, endonucleases, exonucleases, deaminases (e.g., cytidine deaminases or adenosine deaminases), polymerases (e.g., reverse transcriptases), integrases, recombinases, etc., and fusion proteins comprising one or more functional domains linked together.

Ribonucleic acid (RNA) is a molecule that is made up of nucleotides, which are ribose sugars attached to nitrogenous bases and phosphate groups. The nitrogenous bases include adenine (A), guanine (G), uracil (U), and cytosine (C). Generally, RNA mostly exists in the single-stranded form but can also exists double-stranded in certain circumstances. The length, form and structure of RNA is diverse depending on the purpose of the RNA. For example, the length of an RNA can vary from a short sequence (e.g., siRNA) to a long sequences (e.g., lncRNA), can be linear (e.g., mRNA) or circular (e.g., oRNA), and can either be a coding (e.g., mRNA) or a non-coding (e.g., lncRNA) sequence.

In various embodiments, the LNP-based nucleobase editing systems, RNA therapeutics and pharmaceutical compositions thereof described herein can be used to deliver a mRNA payload that is a linear mRNA molecule. In embodiments, the mRNA payload may comprise one or more nucleotide sequences that encode a product of interest, such as, but not limited to a component of a gene editing system (e.g. an endonuclease, a prime editor, etc.) and/or a therapeutic protein.

In some embodiments, the RNA payload may be a linear mRNA. As used herein, the term “messenger RNA” (mRNA) refers to any polynucleotide which encodes a protein of interest and which is capable of being translated to produce the encoded protein of interest in vitro, in vivo, in situ or ex vivo.

Generally, a mRNA molecule comprises at least a coding region, a 5′ untranslated region (UTR), a 3′ UTR, a 5′ cap and a poly-A tail. In some aspects, one or more structural and/or chemical modifications or alterations may be included in the RNA which can reduce the innate immune response of a cell in which the mRNA is introduced. As used herein, a “structural” feature or modification is one in which two or more linked nucleotides are inserted, deleted, duplicated, inverted or randomized in a nucleic acid without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”.

Generally, a coding region of interest in an mRNA used herein may encode a dipeptide, a tripeptide, a tetrapeptide, a pentapeptide, a hexapeptide, a heptapeptide, an octapeptide, a nonapeptide, or a decapeptide. In another embodiment, the mRNA may encode a peptide of 2-30 amino acids, e.g. 5-30, 10-30, 2-25, 5-25, 10-25, or 10-20 amino acids. The mRNA may encode a peptide of at least 10, 11, 12, 13, 14, 15, 17, 20, 25, 26, 27, 28, 29, 30, 31, 32, 33, 34, 35, 36, 37, 38, 39, or 40 amino acids, or a peptide that is no longer than 10, 11, 12, 13, 14, 15, 17, 20, 25, 26, 27, 28, 29, 30, 31, 32, 33, 34, 35, 36, 37, 38, 39, or 40 amino acids.

Generally, the length of the region of the mRNA encoding a product of interest is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the mRNA has a total length that spans from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some embodiments, the region or regions flanking the region encoding the product of interest may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

In some embodiments, the mRNA comprises a tailing sequence which can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

In some embodiments, the mRNA comprises a capping sequence which comprises a single cap or a series of nucleotides forming the cap. The capping sequence may be from 1 to 10, e.g. 2-9, 3-8, 4-7, 1-5, 5-10, or at least 2, or 10 or fewer nucleotides in length. In some embodiments, the caping sequence is absent.

In some embodiments, the mRNA comprises a region comprising a start codon. The region comprising the start codon may range from 3 to 40, e.g., 5-30, 10-20, 15, or at least 4, or 30 or fewer nucleotides in length.

In some embodiments, the mRNA comprises a region comprising a stop codon. The region comprising the stop codon may range from 3 to 40, e.g., 5-30, 10-20, 15, or at least 4, or 30 or fewer nucleotides in length.

In some embodiments, the mRNA comprises a region comprising a restriction sequence. The region comprising the restriction sequence may range from 3 to 40, e.g., 5-30, 10-20, 15, or at least 4, or 30 or fewer nucleotides in length.

### Untranslated Regions (UTRs)

In various embodiments, the mRNA payloads of the LNP-based nucleobase editing systems, RNA therapeutics and pharmaceutical compositions thereof described herein, may comprise at least one untranslated region (UTR) which flanks the region encoding the product of interest and/or is incorporated within the mRNA molecule. UTRs are transcribed by not translated. The mRNA payloads can include 5′ UTR sequences and 3′ UTR sequences, as well as internal UTRs.

The RNA payloads of the present disclosure may comprise one or more regions or parts which act or function as an untranslated region. Where nucleic acids are designed to encode at least one polypeptide of interest, the nucleic acid may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the RNA payload molecules (e.g., linear and circular mRNA molecules) of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

In various embodiments, the mRNA payloads of the LNP-based nucleobase editing systems, RNA therapeutics and pharmaceutical compositions thereof described herein, may comprise at least one UTR that may be selected from any UTR sequence listed in Tables 19 or 20 of U.S. Pat. No. 10,709,779, which is incorporated herein by reference.

### 5′ UTR Regions

In various embodiments, the mRNA payloads of the LNP-based nucleobase editing systems, RNA therapeutics and pharmaceutical compositions thereof described herein, may comprise at least one 5′ UTR.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR (A/G) CCAUGG, where R is a purine (adenine or guanine) three bases upstream of the start codon (AUG), which is followed by another ‘G’. 5′UTR also have been known to form secondary structures which are involved in elongation factor binding. 5′ UTR sequences are also known to be important for ribosome recruitment to the mRNA and have been reported to play a role in translation (Hinnebusch A, et al., (2016) Science, 352:6292: 1413-6). In addition, 5′ UTR sequences may confer increased half-life, increased expression and/or increased activity of a polypeptide encoded by the RNA payload described herein.

In various embodiments, the RNA payload constructs contemplated herein may include 5′UTRs that are found in nature and those that are not. For example, the 5′UTRs can be synthetic and/or can be altered in sequence with respect to a naturally occurring 5′UTR. Such altered 5′UTRs can include one or more modifications relative to a naturally occurring 5′UTR, such as, for example, an insertion, deletion, or an altered sequence, or the substitution of one or more nucleotide analogs in place of a naturally occurring nucleotide.

The 5′ UTR starts at the transcription start site and continues to the start codon but does not include the start codon; whereas, the 3′ UTR starts immediately following the stop codon and continues until the transcriptional termination signal. While not wishing to be bound by theory, the UTRs may have a regulatory role in terms of translation and stability of the nucleic acid.

Natural 5′ UTRs usually include features which have a role in translation initiation as they tend to include Kozak sequences which are commonly known to be involved in the process by which the ribosome initiates translation of many genes. Kozak sequences have the consensus CCR (A/G) CCAUGG, where R is a purine (adenine or guanine) three bases upstream of the start codon (AUG), which is followed by another ‘G’. 5′UTR also have been known to form secondary structures which are involved in elongation factor binding.

In an embodiment, the 5′ UTR comprises a sequence provided in Table X or a sequence having at least 80%, 85%, 90%, 95%, 96%, 97%, 98%, 99% or 100% sequence identity to a 5′ UTR sequence provided in Table X, or a variant or a fragment thereof (e.g., a fragment that lacks the first one, two, three, four, five, or six nucleotides of the 5′ UTR sequence provided in Table Z). In an embodiment, the 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

TABLE Z
Exemplary nucleotide sequences of 5′ UTRs

| | Sequence |
|---|---|
| 5′ UTR Nucleotide Sequence | Identifier |
| ggaaaucgca aaauuugcuc uucgcguuag auuucuuuua guuuucucgc aacuagcaag | SEQ ID NO: 1 |
| cuuuuuguue ucgccgccge c | |
| ggaaaucgca aaauuuucuu uucgcguuag auuucuuuua guuuucuuuc aacuagcaag | SEQ ID NO: 2 |
| cuuuuuguue ucgccgccgc c | |
| ggaaaucgca aaauuuuugc ucuuuuucgc guuagauuuc uuuuaguuuu cuykcaacua | SEQ ID NO: 3 |
| gcaagcuuuu uguucucgce rcc | |
| ggaaaucccc acaaccgccu cauauccagg cucaagaaua gagcucagug uuuuguuguu | SEQ ID NO: 4 |
| uaaucauucc gacguguuuu gcgauauucg cgcaaagcag ccagucgcgc gcuugcuuuu | |
| aaguagaguu guuuuuccac ccguuugcca ggcaucuuua auuuaacaua uuuuuauuuu | |
| ucaggcuaac cuacgccgcc acc | |
| ggaaauaaga gagaaaagaa gaguaagaag aaauauaaga ucucccugag cuucagggag | SEQ ID NO: 5 |
| ccccggegcc gccacc | |
| ggaaaccccc cacccccgua agagagaaaa gaagaguaag aagaaauaua agaucucccu | SEQ ID NO: 6 |
| gagcuucagg gagccccggc gccgccacc | |
| ggagaacuuc cgcuuccguu ggcgcaageg cuuucauuuu uucugcuacc gugacuaag | SEQ ID NO: 7 |
| ggaaauaaga gagaaaagaa gaguaagaag aaauauaaga gccacc | SEQ ID NO: 8 |
| ggaaauaaga gagaaaagaa gaguaagaag aaauauaaga ccccggcgcc gccacc | SEQ ID NO: 9 |
| ggaaacuuua uuuaguguua cuuuauuuuc uguuuauuug uguuucuuca | SEQ ID NO: 10 |
| guggguuugu ucuaauuucc uuggccgcc | |
| ggaaaaucug uauuagguug gcguguuuu uggugguug | SEQ ID NO: 11 |
| uuaguauugu uguugauucg uuuguggucqg guugccgcc | |
| ggaaaauuau uaacaucuug guauucucga uaaccauucg uuggauuuua uuguauucgu | SEQ ID NO: 12 |
| aguuuggguu ccugccgcc | |
| ggaaauuauu auuauuucua guacaauuu aucauuguau uuuuuagcu auucaucauu | SEQ ID NO: 13 |
| auuuacuugg ugaucaaca | |
| ggaaauaggu uguuaaccaa guucaagccu aauaagcuug gauucuggug acuugcuuca | SEQ ID NO: 14 |
| ccguuggcgg gcaccgauc | |
| ggaaaucgua gagagucgua cuuaguacau aucgacuauc gguggacacc aucaagauua | SEQ ID NO: 15 |
| uaaaccaggc caga | |
| ggaaacccgc ccaagcgacc ccaacauauc agcaguugcc caaucccaac ucccaacaca | SEQ ID NO: 16 |
| auccccaagc aacgccgcc | |
| ggaaagcgau ugaaggcguc uuuucaacua cucgauuaag guuggguauc gucgugggac | SEQ ID NO: 17 |
| uuggaaauuu guuguuucc | |
| ggaaacuaau cgaaauaaaa gagccccgua cucuuuuauu ucuauuaggu uaggagccuu | SEQ ID NO: 18 |
| agcauuugua ucuuaggua | |
| ggaaauguga uuuccagcaa cuucuuuuga auauauugaa uuccuaauuc aaagcgaaca | SEQ ID NO: 19 |
| aaucuacaag ccauauacc | |
| ggaaaucgua gagagucgua cuuacguggu cgccauugca uagcgcgcga aagcaacagg | SEQ ID NO: 20 |
| aacaagaacg cgcc | |
| ggaaaucgua gagagucgua cuuagaauaa acagagucgg gucgacuugu cucugauacu | SEQ ID NO: 21 |
| acgacgucac aauc | |
| ggaaaauuug ccuucggagu ugcguauccu gaacugccca gccuccugau auacaacugu | SEQ ID NO: 22 |
| uccgcuuauu cgggccgcc | |
| ggaaaucuga gcaggaaucc uuugugcauu gaagacuuua gauuccucuc ugcgguagac | SEQ ID NO: 23 |
| gugcacuuau aaguauuug | |
| ggaaagcgau ugaaggcguc uuuucaacua cucgauuaag guuggguauc gucgugggac | SEQ ID NO: 24 |
| uuggaaauuu guugccacc | |
| ggaaaauuuu agccuggaac guuagauaac uguccuguug ucuuuauaua cuuggucccc | SEQ ID NO: 25 |
| aaguaguuug ucuuccaaa | |
| ggaaauuuuu uuuugauauu auaagaguuu uuuuuugaua uuaagaaaau | SEQ ID NO: 26 |
| uuuuuuuuga uauuagaaga guaagaagaa auauaagacc ccggcgccgc cacc | |
| ggaaauaaga gagaaaagaa gaguaagaag aaauauaaga gccaaaaaaa aaaaacc | SEQ ID NO: 27 |
| ggaaaucucc cugagcuuca gggaguaaga gagaaaagaa gaguaagaag aaauauaaga | SEQ ID NO: 28 |
| ccccggcgcc gccacc | |

In some embodiments of the disclosure, a 5′ UTR is a heterologous UTR, i.e., is a UTR found in nature associated with a different mRNA. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived alpha-globin or beta-globin (e.g., U.S. Pat. Nos. 8,278,063 and 9,012,219), human cytochrome b-245 polypeptide, and hydroxysteroid (17b) dehydrogenase, and Tobacco etch virus. CMV immediate-early 1 (IE1) gene (scc US20140206753 and WO2013/185069), the sequence GGGAUCCUACC (SEQ ID NO: 29) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738)), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In one embodiment, an internal ribosome entry site (IRES) is used as a substitute for a 5′ UTR.

In some embodiments, a 5′ UTR of the present disclosure comprises SEQ ID NO: 30 (GGGAAAUAAG AGAGAAAAGA AGAGUAAGAA GAAAUAUAAG AGCCACC).

### 3′ UTR Regions

In various embodiments, the mRNA payloads of the LNP-based nucleobase editing systems, RNA therapeutics and pharmaceutical compositions thereof described herein, may comprise at least one 3′ UTR. 3′ UTRs may be heterologous or synthetic.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al., 1995): Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of the mRNA payloads described herein. For example, one or more copies of an ARE can be introduced to make mRNA less stable and thereby curtail translation and decrease production of the resultant protein. Alternatively, AREs can be identified and removed or mutated to increase the intracellular stability and thus increase translation and production of the resultant protein.

In some embodiments, the introduction of features often expressed in genes of target organs the stability and protein production of the mRNA can be enhanced in a specific organ and/or tissue. As a non-limiting example, the feature can be a UTR. As another example, the feature can be introns or portions of introns sequences.

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within an RNA payload construct. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the polypeptide coding region of interest in an mRNA payload may be flanked by a 5′ UTR which may contain a strong Kozak translational initiation signal and/or a 3′ UTR which may include an oligo (dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety

It should be understood that any UTR from any gene may be incorporated into the regions of an RNA payload molecule (e.g., a linear mRNA). Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in U.S. application No. 20090226470, herein incorporated by reference in its entirety, and those known in the art.

### 5′ Capping

In various embodiments, the mRNA payloads of the LNP-based nucleobase editing systems, RNA therapeutics and pharmaceutical compositions thereof described herein, may comprise a 5′ cap structure.

The 5′ cap structure of an mRNA is involved in nuclear export, increasing mRNA stability and binds the mRNA Cap Binding Protein (CBP), which is responsible for mRNA stability in the cell and translation competency through the association of CBP with poly (A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to mRNA may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-0-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-0 atom of the other, unmodified, guanine becomes linked to the 5′-terminal nucleotide of the capped nucleic acid molecule (e.g. an mRNA). The N7- and 3′-0-methlyated guanine provides the terminal moiety of the capped nucleic acid molecule (e.g. mRNA).

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

mRNA may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures are those which, among other things, have enhanced binding of cap binding proteins, increased half-life, reduced susceptibility to 5′ endonucleases and/or reduced 5′decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the CapI structure. This cap results in a higher translational-competency and cellular stability and a reduced activation of cellular pro-inflammatory cytokines, as compared, e.g., to other 5′ cap analog structures known in the art. Cap structures include, but are not limited to, 7mG(5*)ppp(5*)N,pN2p (cap 0), 7mG(5*)ppp(5*)NlmpNp (cap 1), and 7mG(5*)-ppp(5′)NlmpN2mp (cap 2).

In some embodiments, the 5′ terminal caps may include endogenous caps or cap analogs.

In some embodiments, a 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### IRES Sequences

In various embodiments, the mRNA payloads of the LNP-based nucleobase editing systems, RNA therapeutics and pharmaceutical compositions thereof described herein, may comprise one or more IRES sequences.

In some embodiments, the mRNA may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. An mRNA that contains more than one functional ribosome binding site may encode several peptides or polypeptides that are translated independently by the ribosomes. Non-limiting examples of IRES sequences that can be used include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In some embodiments, the IRES is from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human cIF4G, Mouse NDST4L, Human LEF1, Mouse IIIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BNS, Salivirus A BN2, Salivirus A 02394, Salivirus A GUT, Salivirus A CH, Salivirus A SZ1, Salivirus FHB, CVB3, CVB1, Echovirus 7, CVBS, EVA71, CVA3, CVA12, EV24 or an aptamer to eIF4G.

### Poly-A Tails and 3′ Stabilizing Region

In various embodiments, the mRNA payloads of the LNP-based nucleobase editing systems, RNA therapeutics and pharmaceutical compositions thereof described herein, may comprise a poly-A tail.

During RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the free 3′ hydroxyl end. The process, called polyadenylation, adds a poly-A tail of a certain length.

In some embodiments, the length of a poly-A tail is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides) and no more than about 50, 100, 200, 300, 400, 500, 600, 700, 800, 900, 1000, 2000, or 3000 nucleotides in length. In some embodiments, the mRNA includes a poly-A tail from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall mRNA. This design may be based on the length of the region coding for a target of interest, the length of a particular feature or region (such as a flanking region), or based on the length of the ultimate product expressed from the mRNA.

In this context the poly-A tail may be 10, 20, 30, 40, 50, 60, 70, 80, 90, or 100% greater in length than the mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for poly-A binding protein may enhance expression.

Additionally, multiple distinct mRNA may be linked together to the PABP (Poly-A binding protein) through the 3′-end using modified nucleotides at the 3′-terminus of the poly-A tail. Transfection experiments can be conducted in relevant cell lines and protein production can be assayed by ELISA at 12 hr, 24 hr, 48 hr, 72 hr and day 7 post-transfection.

In some embodiments, the mRNA are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail.

### Stop Codons

In various embodiments, the mRNA payloads of the LNP-based nucleobase editing systems, RNA therapeutics and pharmaceutical compositions thereof described herein, may comprise one or more translation stop codons. Translational stop codons, UAA, UAG, and UGA, are an important component of the genetic code and signal the termination of translation of an mRNA. During protein synthesis, stop codons interact with protein release factors and this interaction can modulate ribosomal activity thus having an impact translation (Tate W P, et al., (2018) Biochem Soc Trans, 46(6):1615-162).

A stop element as used herein, refers to a nucleic acid sequence comprising a stop codon. The stop codon can be selected from TGA, TAA and TAG in the case of DNA, or from UGA, UAA and UAG in the case of RNA. In an embodiment, a stop element comprises two consecutive stop codons. In an embodiment, a stop element comprises three consecutive stop codons. In an embodiment, a stop element comprises four consecutive stop codons. In an embodiment, a stop element comprises five consecutive stop codons.

In some embodiments, the mRNA may include one stop codon. In some embodiments, the mRNA may include two stop codons. In some embodiments, the mRNA may include three stop codons. In some embodiments, the mRNA may include at least one stop codon. In some embodiments, the mRNA may include at least two stop codons. In some embodiments, the mRNA may include at least three stop codons. As non-limiting examples, the stop codon may be selected from TGA, TAA and TAG.

In other embodiments, the stop codon may be selected from one or more of the following stop elements of Table Y:

TABLE Y
Additional stop elements
Nucleotide sequence (5' to 3')

| | |
|---|---|
| UGAUAAUAG | UAAAGCUCC |
| UAAUAGUAA | UAAAGCUCC |
| UAAGUCUAA | UAGGGUUAA |
| UAAAGCUAA | UAAGCACCC |
| UAAGUCUCC | UGAUAGUAA |
| UAAGGCUAA | UAAAGCGCU |
| UAAGCCCCU | |

UAAAGCUCCCCGGGG (SEQ ID NO: 31)
UAAGCCCCUCCGGGG (SEQ ID NO: 32)

In some embodiments, the mRNA includes the stop codon TGA and one additional stop codon. In a further embodiment the addition stop codon may be TAA.

### MicroRNA Binding Sites and Other Regulatory Elements

In various embodiments, the mRNA payloads of the LNP-based nucleobase editing systems, RNA therapeutics and pharmaceutical compositions thereof described herein, may comprise one or more regulatory elements, including, but not limited to microRNA (miRNA) binding sites, structured mRNA sequences and/or motifs, artificial binding sites to bind to endogenous nucleic acid binding molecules, and combinations thereof.

### Chemically Unmodified Nucleotides

In some embodiments, the mRNA payloads of the LNP-based nucleobase editing systems, RNA therapeutics and pharmaceutical compositions thereof described herein are not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemically Modified Nucleotides

In some embodiments, the mRNA payloads of the LNP-based nucleobase editing systems, RNA therapeutics and pharmaceutical compositions thereof described herein comprise, in some embodiments, comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m¹ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m¹ψ), 5-methoxy-uridine (mo⁵U), 5-methyl-cytidine (m⁵C), pseudouridine (ψ), α-thio-guanosine and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (ψ) and 5-methyl-cytidine (m⁵C). In some embodiments, polynucleotides (e.g., RNA polynucleotides, such as mRNA polynucleotides) comprise 1-methyl-pseudouridine (m1w). In some embodiments, polynucleotides (e.g., RNA polynucleotides, such as mRNA polynucleotides) comprise 1-methyl-pseudouridine (m¹ψ) and 5-methyl-cytidine (m⁵C). In some embodiments, polynucleotides (e.g., RNA polynucleotides, such as mRNA polynucleotides) comprise 2-thiouridine (s²U). In some embodiments, polynucleotides (e.g., RNA polynucleotides, such as mRNA polynucleotides) comprise 2-thiouridine and 5-methyl-cytidine (m⁵C). In some embodiments, polynucleotides (e.g., RNA polynucleotides, such as mRNA polynucleotides) comprise methoxy-uridine (mo⁵U). In some embodiments, polynucleotides (e.g., RNA polynucleotides, such as mRNA polynucleotides) comprise 5-methoxy-uridine (mo⁵U) and 5-methyl-cytidine (m⁵C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m⁵C). In some embodiments, polynucleotides (e.g., RNA polynucleotides, such as mRNA polynucleotides) comprise N6-methyl-adenosine (m⁶A). In some embodiments, polynucleotides (e.g., RNA polynucleotides, such as mRNA polynucleotides) comprise N6-methyl-adenosine (m⁶A) and 5-methyl-cytidine (mC).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m⁵C), meaning that all cytosine residues in the mRNA sequence are replaced with 5-methyl-cytidine (m⁵C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and In some embodiments, a modified nucleobase is a modified cytosine. nucleosides having a modified uridine include 5-cyano uridine, and 4′-thio uridine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the present disclosure, or in a given predetermined sequence region thereof (e.g., in the mRNA including or excluding the poly A tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

D. Circular mRNA Payloads

In various embodiments, the LNP-based nucleobase editing systems, RNA therapeutics and pharmaceutical compositions thereof described herein can be used to deliver an RNA payload that is a circular mRNA molecule or “ORNA.” The circular mRNA molecule may encode a CROI, such as a nucleobase editing system, or therapeutic protein as described in this specification.

In various embodiments, the LNP-based pharmaceutical compositions described herein, e.g., LNP-based gene editing systems, may include one or more circular mRNA molecules or “ORNAs.” In various embodiments, the circular mRNA payloads may encode one or more components of the herein described gene editing systems or other therapeutic protein of interest. For example, a circular mRNA payload may encode an amino acid sequence-programmable DNA binding domain (e.g., TALENS and zinc finger-binding domains) or a nucleic acid sequence-programmable DNA binding domain (e.g., CRISPR Cas9, CRISPR Cas12a, CRISPR Cas12f, CRISPR Cas13a, CRISPR Cas13b, or TnpB).

The circular mRNA payloads may also encode, depending upon the nature of the gene editing system, one or more effector domains that provide various functionalities that facilitate changes in nucleotide sequence and/or gene expression, such as, but not limited to, single-strand DNA binding proteins, nucleases, endonucleases, exonucleases, deaminases (e.g., cytidine deaminases or adenosine deaminases), polymerases (e.g., reverse transcriptases), integrases, recombinases, etc., and fusion proteins comprising one or more functional domains linked together.

In some embodiments, the RNA payload is a circular RNA (ORNA). As used herein, the terms “ORNA” or “circular RNA” are used interchangeably and can refer to a RNA that forms a circular structure through covalent or non-covalent bonds.

Circular RNA described herein are polyribonucleotides that form a continuous structure through covalent or non-covalent bonds. Due to the circular structure, oRNAs have improved stability, increased half-life, reduced immunogenicity, and/or improved functionality (e.g., of a function described herein) compared to a corresponding linear RNA.

In some embodiments, an oRNA binds a target. In some embodiments, an oRNA binds a substrate. In some embodiments, an oRNA binds a target and binds a substrate of the target. In some embodiments, an oRNA binds a target and mediates modulation of a substrate of the target. In some embodiments, an oRNA brings together a target and its substrate to mediate modification of the substrate, e.g., post-translational modification. In some embodiments, an oRNA brings together a target and its substrate to mediate a cellular process (e.g., alters protein degradation or signal transduction) involving the substrate. In some embodiments, a target is a target protein and a substrate is a substrate protein.

In some embodiments, an oRNA comprises a conjugation moiety for binding to chemical compound. The conjugation moiety can be a modified polyribonucleotide. The chemical compound can be conjugated to the oRNA by the conjugation moiety. In some embodiments, the chemical compound binds to a target and mediates modulation of a substrate of the target. In some embodiments, an oRNA binds a substrate of a target and a chemical compound conjugated to the oRNA by the conjugation moiety binds the target to bring together the target and its substrate to mediate modification of the substrate, e.g., post-translational modification. In some embodiments, an ORNA binds a substrate of a target and a chemical compound conjugated to the oRNA by the conjugation moiety binds the target to bring together the target and its substrate to mediate modification of the substrate to mediate a cellular process (e.g., alters protein degradation or signal transduction) involving the substrate. In some embodiments, a target is a target protein and a substrate is a substrate protein.

In some embodiments, the oRNA may be non-immunogenic in a mammal (e.g., a human, non-human primate, rabbit, rat, and mouse).

In some embodiments, the oRNA may be capable of replicating or replicates in a cell from an aquaculture animal (e.g., fish, crabs, shrimp, oysters etc.), a mammalian cell, a cell from a pet or zoo animal (e.g., cats, dogs, lizards, birds, lions, tigers and bears etc.), a cell from a farm or working animal (e.g., horses, cows, pigs, chickens etc.), a human cell, cultured cells, primary cells or cell lines, stem cells, progenitor cells, differentiated cells, germ cells, cancer cells (e.g., tumorigenic, metastatic), non-tumorigenic cells (e.g., normal cells), fetal cells, embryonic cells, adult cells, mitotic cells, non-mitotic cells, or any combination thereof.

In one aspect, provided herein is a pharmaceutical composition comprising: a circular RNA comprising, in the following order, a 3′ group I intron fragment, an Internal Ribosome Entry Site (IRES), an expression sequence encoding a polypeptide (e.g., a nucleobase editing system, therapeutic protein), and a 5′ group I intron fragment, and a transfer vehicle comprising at least one of (i) an ionizable lipid, (ii) a structural lipid, and (iii) a PEG-modified lipid, wherein the transfer vehicle is capable of delivering the circular RNA polynucleotide to a cell (e.g., a human cell, such as an immune cell present in a human subject), such that the polypeptide is translated in the cell.

In some embodiments, the pharmaceutical composition is formulated for intravenous administration to the human subject in need thereof. In some embodiments, the 3′ group I intron fragment and 5′ group I intron fragment are Anabaena group I intron fragments.

In certain embodiments, the 3′ intron fragment and 5′ intron fragment are defined by the L9a-5 permutation site in the intact intron. In certain embodiments, the 3′ intron fragment and 5′ intron fragment are defined by the L8-2 permutation site in the intact intron.

In some embodiments, the IRES is from Taura syndrome virus, Tiiatoma virus, Theiler's encephalomyelitis virus, Simian Virus 40, Solenopsis invicta virus 1, Rhopalosiphum padi virus, Reticuloendotheliosis virus, Human poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picoma-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIFI alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SHI, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA 16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistravirus, Hubei Picoma-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the IRES comprises a CVB3 IRES or a fragment or variant thereof. In some embodiments, the pharmaceutical composition comprises a first internal spacer between the 3′ group I intron fragment and the IRES, and a second internal spacer between the expression sequence and the 5′ group I intron fragment. In certain embodiments, the first and second internal spacers each have a length of about 10 to about 60 nucleotides.

In some embodiments, the circular mRNA comprises a nucleotide sequence encoding a polypeptide of interest, such as a nucleobase editing system or therapeutic protein.

In some embodiments, the LNP-based nucleobase editing systems, RNA therapeutics and pharmaceutical compositions described herein are administered in an amount effective to treat a disease in the human subject (e.g., wherein the disease can be cancer, muscle disorder, or CNS disorder, etc.). In some embodiments, the LNP-based nucleobase editing systems, RNA therapeutics and pharmaceutical compositions have an enhanced safety profile when compared to a pharmaceutical composition comprising T cells or vectors comprising exogenous DNA encoding the same polypeptide, e.g., a CAR complex protein.

In some embodiments, the LNP-based nucleobase editing systems and pharmaceutical compositions thereof are administered in an amount effective to mount an immunogenic response in a human subject for the vaccination against an infectious agent and/or cancer. In some embodiments, the LNP-based nucleobase editing systems and pharmaceutical compositions have an enhanced safety profile when compared to state of the art gene editing delivery compositions.

In another aspect, the present disclosure provides a circular RNA comprising, in the following order, a 3′ group I intron fragment, an Internal Ribosome Entry Site (IRES), an expression sequence encoding a polypeptide (e.g., a nucleobase editing system, therapeutic protein, such as a chimeric antigen receptor (CAR) or T cell receptor (TCR) complex protein), and a 5′ group I intron fragment.

In some embodiments, the 3′ group I intron fragment and 5′ group I intron fragment are Anabaena group I intron fragments. In certain embodiments, the 3′ intron fragment and 5′ intron fragment are defined by the L9a-5 permutation site in the intact intron. In certain embodiments, the 3′ intron fragment and 5′ intron fragment are defined by the L8-2 permutation site in the intact intron. In certain embodiments, the IRES comprises a CVB3 IRES or a fragment or variant thereof.

In some embodiments, the circular RNA comprises a first internal spacer between the 3′ group I intron fragment and the IRES, and a second internal spacer between the expression sequence and the 5′ group I intron fragment.

In certain embodiments, the first and second internal spacers each have a length of about 10 to about 60 nucleotides.

In some embodiments, the circular RNA payload of the LNP-based nucleobase editing systems, RNA therapeutics and pharmaceutical compositions described herein consists of natural nucleotides. In some embodiments, the circular RNA further comprises a second expression sequence encoding a therapeutic protein. In some embodiments, the therapeutic protein comprises a checkpoint inhibitor. In certain embodiments, the therapeutic protein comprises a cytokine.

In some embodiments, the circular RNA payload of the LNP-based nucleobase editing systems, RNA therapeutics and pharmaceutical compositions described herein consists of natural nucleotides.

In some embodiments, the circular RNA payload LNP-based nucleobase editing systems, RNA therapeutics and pharmaceutical compositions described herein comprises a nucleotide sequence that is codon optimized, either partially or fully. In some embodiments, the circular RNA is optimized to lack at least one microRNA binding site present in an equivalent pre-optimized polynucleotide. In some embodiments, the circular RNA is optimized to lack at least one endonuclease susceptible site present in an equivalent pre-optimized polynucleotide. In some embodiments, the circular RNA is optimized to lack at least one RNA-editing susceptible site present in an equivalent pre-optimized polynucleotide.

In some embodiments, the circular RNA payload of the LNP-based nucleobase editing systems, RNA therapeutics and pharmaceutical compositions described herein has an in vivo functional half-life in humans greater than that of an equivalent linear RNA having the same expression sequence. In some embodiments, the circular RNA has a length of about 100 nucleotides to about 10 kilobases. In some embodiments, the circular RNA has a functional half-life of at least about 20 hours. In some embodiments, the circular RNA has a duration of therapeutic effect in a human cell of at least about 20 hours. In some embodiments, the circular RNA has a duration of therapeutic effect in a human cell greater than or equal to that of an equivalent linear RNA comprising the same expression sequence. In some embodiments, the circular RNA has a functional half-life in a human cell greater than or equal to that of an equivalent linear RNA comprising the same expression sequence.

In some embodiments, the circular RNA payload of the LNP-based nucleobase editing systems, RNA therapeutics and pharmaceutical compositions described herein has a half-life of at least that of a linear counterpart. In some embodiments, the oRNA has a half-life that is increased over that of a linear counterpart. In some embodiments, the half-life is increased by about 5%, 10%, 15%, 20%, 25%, 30%, 35%, 40%, 45%, 50%, or greater. In some embodiments, the oRNA has a half-life or persistence in a cell for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In some embodiments, the oRNA has a half-life or persistence in a cell for no more than about 10 mins to about 7 days, or no more than about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 24 hours (1 day), 36 hours (1.5 days), 48 hours (2 days), 60 hours (2.5 days), 72 hours (3 days), 4 days, 5 days, 6 days, or 7 days.

In some embodiments, the circular RNA payload of the LNP-based nucleobase editing systems, RNA therapeutics and pharmaceutical compositions described herein has a half-life or persistence in a cell while the cell is dividing. In some embodiments, the oRNA has a half-life or persistence in a cell post division.

In certain embodiments, the circular RNA payload of the LNP-based nucleobase editing systems, RNA therapeutics and pharmaceutical compositions described herein has a half-life or persistence in a dividing cell for greater than about 10 minutes to about 30 days, or at least about 10 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the circular RNA payload of the LNP-based nucleobase editing systems, RNA therapeutics and pharmaceutical compositions described herein modulates a cellular function, e.g., transiently or long term. In certain embodiments, the cellular function is stably altered, such as a modulation that persists for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer. In certain embodiments, the cellular function is transiently altered, e.g., such as a modulation that persists for no more than about 30 mins to about 7 days, or no more than about 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours (1 day), 36 hours (1.5 days), 48 hours (2 days), 60 hours (2.5 days), 72 hours (3 days), 4 days, 5 days, 6 days, or 7 days.

In some embodiments, the circular RNA payload of the LNP-based nucleobase editing systems, RNA therapeutics and pharmaceutical compositions described herein is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the oRNA may be of a sufficient size to accommodate a binding site for a ribosome.

In some embodiments, the maximum size of the circular RNA payload of the LNP-based nucleobase editing systems, RNA therapeutics and pharmaceutical compositions described herein may be limited by the ability of packaging and delivering the RNA to a target. In some embodiments, the size of the oRNA is a length sufficient to encode polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides may be useful.

In some embodiments, the circular RNA payload of the LNP-based nucleobase editing systems, RNA therapeutics and pharmaceutical compositions described herein comprises one or more elements described elsewhere herein. In some embodiments, the elements may be separated from one another by a spacer sequence or linker. In some embodiments, the elements may be separated from one another by 1 nucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides.

In some embodiments, one or more elements are contiguous with one another, e.g., lacking a spacer element.

In some embodiments, one or more elements is conformationally flexible. In some embodiments, the conformational flexibility is due to the sequence being substantially free of a secondary structure.

In some embodiments, the circular RNA payload of the LNP-based nucleobase editing systems, RNA therapeutics and pharmaceutical compositions described herein comprises a secondary or tertiary structure that accommodates a binding site for a ribosome, translation, or rolling circle translation.

In some embodiments, the circular RNA payload of the LNP-based nucleobase editing systems, RNA therapeutics and pharmaceutical compositions described herein comprises particular sequence characteristics. For example, the oRNA may comprise a particular nucleotide composition. In some such embodiments, the oRNA may include one or more purine rich regions (adenine or guanosine). In some such embodiments, the oRNA may include one or more purine rich regions (adenine or guanosine). In some embodiments, the oRNA may include one or more AU rich regions or elements (AREs). In some embodiments, the oRNA may include one or more adenine rich regions.

In some embodiments, the circular RNA payload of the LNP-based nucleobase editing systems, RNA therapeutics and pharmaceutical compositions described herein comprises one or more modifications described elsewhere herein.

In some embodiments, the circular RNA payload of the LNP-based nucleobase editing systems, RNA therapeutics and pharmaceutical compositions described herein comprises one or more expression sequences and is configured for persistent expression in a cell of a subject in vivo. In some embodiments, the oRNA is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Regulatory Elements

In some embodiments, the circular RNA payload of the LNP-based nucleobase editing systems, RNA therapeutics and pharmaceutical compositions described herein comprises one or more regulatory elements. As used herein, a “regulatory element” is a sequence that modifies expression of an expression sequence, e.g., a nucleotide sequence encoding a nucleobase editing system or a therapeutic protein, i.e., a coding region of interest (CROI). The regulatory element may include a sequence that is located adjacent to a coding region of interest encoded on the circular RNA payload. The regulatory element may be operatively linked to a nucleotide sequence of the circular RNA that encodes a coding region of interest (e.g., a nucleobase editing system or therapeutic polypeptide).

In some embodiments, a regulatory element may increase an amount of expression of a coding region of interest encoded on the circular RNA payload as compared to an amount expressed when no regulatory element exists.

In some embodiments, a regulatory element may comprise a sequence to selectively initiates or activates translation of a coding sequence of interest encoded on the circular RNA payload.

In some embodiments, a regulatory element may comprise a sequence to initiate degradation of the oRNA or the payload or cargo. Non-limiting examples of the sequence to initiate degradation includes, but is not limited to, riboswitch aptazyme and miRNA binding sites.

In some embodiments, a regulatory element can modulate translation of a coding region of interest encoded on the oRNA. The modulation can create an increase (enhancer) or decrease (suppressor) in the expression of the coding region of interest. The regulatory element may be located adjacent to the CROI (e.g., on one side or both sides of the CROI).

### Translation Initiation Sequence

In some embodiments, a translation initiation sequence functions as a regulatory element. In some embodiments, the translation initiation sequence comprises an AUG/ATG codon. In some embodiments, a translation initiation sequence comprises any eukaryotic start codon such as, but not limited to, AUG/ATG, CUG/CTG, GUG/GTG, UUG/TTG, ACG, AUC/ATC, AUU, AAG, AUA/ATA, or AGG. In some embodiments, a translation initiation sequence comprises a Kozak sequence. In some embodiments, translation begins at an alternative translation initiation sequence, e.g., translation initiation sequence other than AUG/ATG codon, under selective conditions, e.g., stress induced conditions. As a non-limiting example, the translation of the circular polyribonucleotide may begin at alternative translation initiation sequence, such as ACG. As another non-limiting example, the circular polyribonucleotide translation may begin at alternative translation initiation sequence, CUG/CTG. As another non-limiting example, the translation may begin at alternative translation initiation sequence, GUG/GTG. As yet another non-limiting example, the translation may begin at a repeat-associated non-AUG (RAN) sequence, such as an alternative translation initiation sequence that includes short stretches of repetitive RNA e.g. CGG, GGGGCC, CAG, CTG.

In some embodiments, the oRNA encodes a polypeptide or peptide and may comprise a translation initiation sequence. The translation initiation sequence may comprise, but is not limited to a start codon, a non-coding start codon, a Kozak sequence or a Shine-Dalgarno sequence. The translation initiation sequence may be located adjacent to the payload or cargo (e.g., on one side or both sides of the coding region of interest).

In some embodiments, the translation initiation sequence provides conformational flexibility to the oRNA. In some embodiments, the translation initiation sequence is within a substantially single stranded region of the oRNA.

The ORNA may include more than 1 start codon such as, but not limited to, at least 2, at least 3, at least 4, at least 5, at least 6, at least 7, at least 8, at least 9, at least 10, at least 11, at least 12, at least 13, at least 14, at least 15 or more than 15 start codons. Translation may initiate on the first start codon or may initiate downstream of the first start codon.

In some embodiments, the oRNA may initiate at a codon which is not the first start codon, e.g., AUG. Translation of the circular polyribonucleotide may initiate at an alternative translation initiation sequence, such as, but not limited to, ACG, AGG, AAG, CUG/CTG, GUG/GTG, AUA/ATA, AUU/ATT, UUG/TTG. In some embodiments, translation begins at an alternative translation initiation sequence under selective conditions, e.g., stress induced conditions. As a non-limiting example, the translation of the oRNA may begin at alternative translation initiation sequence, such as ACG. As another non-limiting example, the oRNA translation may begin at alternative translation initiation sequence, CUG/CTG. As yet another non-limiting example, the oRNA translation may begin at alternative translation initiation sequence, GTG/GUG. As yet another non-limiting example, the oRNA may begin translation at a repeat-associated non-AUG (RAN) sequence, such as an alternative translation initiation sequence that includes short stretches of repetitive RNA e.g. CGG, GGGGCC, CAG, CTG.

### IRES Sequences

In some embodiments, the oRNA described herein comprises an internal ribosome entry site (IRES) element capable of engaging an eukaryotic ribosome. In some embodiments, the IRES element is at least about 5 nucleotides, at least about 8 nucleotides, at least about 9 nucleotides, at least about 10 nucleotides, at least about 15 nucleotides, at least about 20 nucleotides, at least about 25 nucleotides, at least about 30 nucleotides, at least about 40 nucleotides, at least about 50 nucleotides, at least about 100 nucleotides, at least about 200 nucleotides, at least about 250 nucleotides, at least about 350 nucleotides, or at least about 500 nucleotides. In one embodiment, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES element is at least partially derived from a virus, for instance, it can be derived from a viral IRES element, such as ABPV_IGRpred, AEV, ALPV_IGRpred, BQCV_IGRpred, BVDV1_1-385, BVDV1_29-391, CrPV_5NCR, CrPV_IGR, crTMV_IREScp, crTMV_IRESmp75, crTMV_IRESmp228, crTMV_IREScp, crTMV_IREScp, CSFV, CVB3, DCV_IGR, EMCV-R, EoPV_5NTR, ERAV 245-961, ERBV 162-920, EV71_1-748, FeLV-Notch2, FMDV_type_C, GBV-A, GBV-B, GBV-C, gypsy_env, gypsyD5, gypsyD2, HAV_HM175, HCV_type_1a, HiPV_IGRpred, HIV-1, HoCV1_IGRpred, HRV-2, IAPV_IGRpred, idefix, KBV_IGRpred, LINE-1_ORF1_-101_to_-1, LINE-1_ORF1-302_to_-202, LINE-1_ORF2-138_to_-86, LINE-1_ORF1_-44to_-1, PSIV_IGR, PV_type1_Mahoney, PV_type3_Leon, REV-A, RhPV_5NCR, RhPV_IGR, SINV1_IGRpred, SV40_661-830, TMEV, TMV_UI_IRESmp228, TRV_5NTR, TrV_IGR, or TSV_IGR. In some embodiments, the IRES element is at least partially derived from a cellular IRES, such as AML1/RUNX1, Antp-D, Antp-DE, Antp-CDE, Apaf-1, Apaf-1, AQP4, ATIR_var1, ATIR_var2, ATIR_var3, AT1R_var4, BAG1_p36delta236 nt, BAG1_p36, BCL2, BiP_-222_-3, c-IAP1_285-1399, c-IAP1_1313-1462, c-jun, c-myc, Cat-1224, CCND1, DAPS, cIF4G, cIF4GI-ext, cIF4GII, cIF4GII-long, ELG1, ELH, FGF1A, FMR1, Gtx-133-141, Gtx-1-166, Gtx-1-120, Gtx-1-196, hairless, IIAP4, IIIF1a, hSNM1, HIsp101, hsp70, hsp70, HIsp90, IGF2_leader2, Kv1.4_1.2, L-myc, LamB1_-335_-1, LEF1, MNT_75-267, MNT_36-160, MTG8a, MYB, MYT2_997-1152, n-MYC, NDST1, NDST2, NDST3, NDST4L, NDST4S, NRF_-653_-17, NtHSF1, ODC1, p27kip1, 03_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A-133-1, XIAP_5-464, XIAP_305-466, or YAP1.

In another embodiment, the IRES is an IRES sequence from Coxsackievirus B3 (CVB3), the protein coding region encodes Guassia luciferase (Gluc) and the spacer sequences are polyA-C.

In some embodiments, the IRES, if present, is at least about 50 nucleotides in length. In one embodiment, the vector comprises an IRES that comprises a natural sequence. In one embodiment, the vector comprises an IRES that comprises a synthetic sequence.

An IRES may act as the sole ribosome binding site, or may serve as one of multiple ribosome binding sites of an mRNA. A polynucleotide containing more than one functional ribosome binding site may encode several peptides or polypeptides that are translated independently by the ribosomes (e.g., multicistronic mRNA). When polynucleotides are provided with an IRES, further optionally provided is a second translatable region. Examples of IRES sequences that can be used according to the present disclosure include without limitation, those from picornaviruses (e.g., FMDV), pest viruses (CFFV), polio viruses (PV), encephalomyocarditis viruses (ECMV), foot- and mouth disease viruses (FMDV), hepatitis C viruses (HCV), classical Swine fever viruses (CSFV), murine leukemia virus (MLV), simian immune deficiency viruses (SIV) or cricket paralysis viruses (CrPV).

### Termination Element

In some embodiments, the oRNA includes one or more coding regions of interest (i.e., also referred to as product expression sequences) which encode polypeptides of interest, including but not limited to nucleobase editing system and therapeutic proteins. In various embodiments, the product expression sequences may or may not have a termination element.

In some embodiments, the oRNA includes one or more product expression sequences that lack a termination element, such that the oRNA is continuously translated.

Exclusion of a termination element may result in rolling circle translation or continuous expression of the encoded peptides or polypeptides as the ribosome will not stall or fall-off. In such an embodiment, rolling circle translation expresses continuously through the product expression sequence.

In some embodiments, one or more product expression sequences in the oRNA comprise a termination element.

In some embodiments, not all of the product expression sequences in the oRNA comprise a termination element. In such instances, the product expression sequence may fall off the ribosome when the ribosome encounters the termination element and terminates translation.

### Rolling Circle Translation

In some embodiments, once translation of the oRNA is initiated, the ribosome bound to the ORNA does not disengage from the oRNA before finishing at least one round of translation of the ORNA. In some embodiments, the oRNA as described herein is competent for rolling circle translation. In some embodiments, during rolling circle translation, once translation of the oRNA is initiated, the ribosome bound to the oRNA does not disengage from the oRNA before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1,000 rounds, at least 1,500 rounds, at least 2,000 rounds, at least 5,000 rounds, at least 10,000 rounds, at least 103 rounds, or at least 106 rounds of translation of the oRNA.

In some embodiments, the rolling circle translation of the oRNA leads to generation of polypeptide that is translated from more than one round of translation of the oRNA. In some embodiments, the oRNA comprises a stagger element, and rolling circle translation of the oRNA leads to generation of polypeptide product that is generated from a single round of translation or less than a single round of translation of the oRNA.

### Circularization

In one embodiment, a linear RNA may be cyclized, or concatemerized. In some embodiments, the linear RNA may be cyclized in vitro prior to formulation and/or delivery. In some embodiments, the linear RNA may be cyclized within a cell.

In some embodiments, the mechanism of cyclization or concatemerization may occur through at least 3 different routes: 1) chemical, 2) enzymatic, and 3) ribozyme catalyzed. The newly formed 5′-/3′-linkage may be intramolecular or intermolecular.

In the first route, the 5′-end and the 3′-end of the nucleic acid contain chemically reactive groups that, when close together, form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a synthetic mRNA molecule will undergo a nucleophilic attack on the 5′-NHS-ester moiety forming a new 5′-/3′-amide bond.

In the second route, T4 RNA ligase may be used to enzymatically link a 5′-phosphorylated nucleic acid molecule to the 3′-hydroxyl group of a nucleic acid forming a new phosphorodiester linkage. In an example reaction, \hat{ }g of a nucleic acid molecule is incubated at 37° C. for 1 hour with 1-10 units of T4 RNA ligase (New England Biolabs, Ipswich, MA) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of the cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme may be derived from the Group I Intron, Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, the oRNA is made via circularization of a linear RNA.

In some embodiments, the following elements are operably connected to each other and, in some embodiments, arranged in the following sequence: a.) a 5′ homology arm, b.) a 3′ group I intron fragment containing a 3′ splice site dinucleotide, c.) a protein coding or noncoding region, d.) a 5′ group I intron fragment containing a 5′ splice site dinucleotide, and e.) a 3′ homology arm. In certain embodiments said vector allows production of a circular RNA that is translatable and/or biologically active inside eukaryotic cells. In some embodiments, the biologically active RNA is, for example, an miRNA sponge, or long noncoding RNA.

In some embodiments, the following elements are operably connected to each other and arranged in the following sequence: a.) a 5′ homology arm, b.) a 3′ group I intron fragment containing a 3′ splice site dinucleotide, c.) optionally, a 5′ spacer sequence, d.) optionally, an internal ribosome entry site (IRES), e.) a protein coding or noncoding region, f.) optionally, a 3′ spacer sequence, g.) a 5′ group I intron fragment containing a 5′ splice site dinucleotide, and h.) a 3′ homology arm. In certain embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) an internal ribosome entry site (IRES), c.) a protein coding or noncoding region, f.) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) a protein coding or noncoding region, e.) a 3′ spacer sequence, f.) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) an internal ribosome entry site (IRES), d.) a protein coding or noncoding region, c.) a 3′ spacer sequence, f) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a protein coding or noncoding region, d.) a 3′ spacer sequence, e.) a 5′ group I intron fragment containing a 5′ splice site dinucleotide, and f.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) a protein coding or noncoding region, e.) a 5′ group I intron fragment containing a 5′ splice site dinucleotide, and f.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) an internal ribosome entry site (IRES), d.) a protein coding or noncoding region, e.) a 5′ group I intron fragment containing a 5′ splice site dinucleotide, and f) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) an internal ribosome entry site (IRES), e.) a protein coding or noncoding region, f) a 3′ spacer sequence, g.) a 5′ group I intron fragment containing a 5′ splice site dinucleotide, and h.) a 3′ homology arm. In some embodiments, said vector allowing production of a circular RNA that is translatable and/or biologically active inside eukaryotic cells.

In one embodiment, the 3′ group I intron fragment and/or the 5′ group I intron fragment is from a Cyanobacterium Anabaena sp. pre-tRNA-Leu gene or T4 phage Td gene.

In one embodiment, the 3′ group I intron fragment and/or the 5′ group I intron fragment is from a Cyanobacterium Anabaena sp. pre-tRNA-Leu gene.

In one embodiment, the protein coding region encodes a protein of eukaryotic or prokaryotic origin. In another embodiment, the protein coding region encodes human protein or non-human protein. In some embodiments, the protein coding region encodes one or more antibodies. For example, in some embodiments, the protein coding region encodes human antibodies. In one embodiment, the protein coding region encodes a protein selected from hFIX, SP-B, VEGF-A, human methylmalonyl-CoA mutase (hMUT), CFTR, cancer self-antigens, and additional gene editing enzymes like Cpf1, zinc finger nucleases (ZFNs) and transcription activator-like effector nucleases (TALENs). In another embodiment, the protein coding region encodes a protein for therapeutic use. In one embodiment, the human antibody encoded by the protein coding region is an anti-HIV antibody. In one embodiment, the antibody encoded by the protein coding region is a bispecific antibody. In one embodiment, the bispecific antibody is specific for CD19 and CD22. In another embodiment, the bispecific antibody is specific for CD3 and CLDN6. In one embodiment, the protein coding region encodes a protein for diagnostic use. In one embodiment, the protein coding region encodes Gaussia luciferase (Gluc), Firefly luciferase (Fluc), enhanced green fluorescent protein (eGFP), human erythropoietin (hEPO), or Cas9 endonuclease.

In one embodiment, the 5′ homology arm is about 5-50 nucleotides in length. In another embodiment, the 5′ homology arm is about 9-19 nucleotides in length. In some embodiments, the 5′ homology arm is at least 5, 6, 7, 8, 9, 10, 11, 12, 13, 14, 15, 16, 17, 18 or 19 nucleotides in length. In some embodiments, the 5′ homology arm is no more than 50, 45, 40, 35, 30, 25 or 20 nucleotides in length. In some embodiments, the 5′ homology arm is 5, 6, 7, 8, 9, 10, 11, 12, 13, 14, 15, 16, 17, 18 or 19 nucleotides in length.

In one embodiment, the 3′ homology arm is about 5-50 nucleotides in length. In another embodiment, the 3′ homology arm is about 9-19 nucleotides in length. In some embodiments, the 3′ homology arm is at least 5, 6, 7, 8, 9, 10, 11, 12, 13, 14, 15, 16, 17, 18 or 19 nucleotides in length. In some embodiments, the 3′ homology arm is no more than 50, 45, 40, 35, 30, 25 or 20 nucleotides in length. In some embodiments, the 3′ homology arm is 5, 6, 7, 8, 9, 10, 11, 12, 13, 14, 15, 16, 17, 18 or 19 nucleotides in length.

In one embodiment, the 5′ spacer sequence is at least 10 nucleotides in length. In another embodiment, the 5′ spacer sequence is at least 15 nucleotides in length. In a further embodiment, the 5′ spacer sequence is at least 30 nucleotides in length. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyA-C sequence.

In one embodiment, the 3′ spacer sequence is at least 10 nucleotides in length. In another embodiment, the 3′ spacer sequence is at least 15 nucleotides in length. In a further embodiment, the 3′ spacer sequence is at least 30 nucleotides in length. In some embodiments, the 3′ spacer sequence is at least 7, 8, 9, 10, 11, 12, 13, 14, 15, 16, 17, 18, 19, 20, 25 or 30 nucleotides in length. In some embodiments, the 3′ spacer sequence is no more than 100, 90, 80, 70, 60, 50, 45, 40, 35 or 30 nucleotides in length. In some embodiments the 3′ spacer sequence is between 20 and 50 nucleotides in length. In certain embodiments, the 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 3′ spacer sequence is a polyA sequence. In another embodiment, the 5′ spacer sequence is a polyA-C sequence.

### Extracellular Circularization

In some embodiments, the linear RNA is cyclized, or concatemerized using a chemical method to form an oRNA. In some chemical methods, the 5′-end and the 3′-end of the nucleic acid (e.g., a linear RNA) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA will undergo a nucleophilic attack on the 5′-NHS-ester moiety forming a new 5′-/3′-amide bond.

In one embodiment, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphorodiester linkage. In an example reaction, a linear RNA is incubated at 37 C for 1 hour with 1-10 units of T4 RNA ligase according to the manufacturer's protocol. The ligation reaction may occur in the presence of a linear nucleic acid capable of base-pairing with both the 5′- and 3′-region in juxtaposition to assist the enzymatic ligation reaction. In one embodiment, the ligation is splint ligation where a single stranded polynucleotide (splint), like a single stranded RNA, can be designed to hybridize with both termini of a linear RNA, so that the two termini can be juxtaposed upon hybridization with the single-stranded splint. Splint ligase can thus catalyze the ligation of the juxtaposed two termini of the linear RNA, generating an oRNA.

In one embodiment, a DNA or RNA ligase may be used in the synthesis of the oRNA. As a non-limiting example, the ligase may be a circ ligase or circular ligasc.

In one embodiment, either the 5′- or 3′-end of the linear RNA can encode a ligase ribozyme sequence such that during in vitro transcription, the resultant linear RNA includes an active ribozyme sequence capable of ligating the 5′-end of the linear RNA to the 3′-end of the linear RNA. The ligase ribozyme may be derived from the Group I Intron, Hepatitis Delta Virus, Hairpin ribozyme or may be selected by SELEX (systematic evolution of ligands by exponential enrichment).

In one embodiment, a linear RNA may be cyclized or concatemerized by using at least one non-nucleic acid moiety. In one aspect, the at least one non-nucleic acid moiety may react with regions or features near the 5′ terminus and/or near the 3′ terminus of the linear RNA in order to cyclize or concatermerize the linear RNA. In another aspect, the at least one non-nucleic acid moiety may be located in or linked to or near the 5′ terminus and/or the 3′ terminus of the linear RNA. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In one embodiment, a linear RNA may be cyclized or concatemerized due to a non-nucleic acid moiety that causes an attraction between atoms, molecular surfaces at, near or linked to the 5′ and 3′ ends of the linear RNA. As a non-limiting example, one or more linear RNA may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In one embodiment, the linear RNA may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear RNA to cyclize or concatemerize. In another aspect, the peptides covalently linked to the ribozyme RNA near the 5′ terminus and the 3′ terminus may cause the linear RNA to cyclize or concatemerize after being subjected to ligated using various methods known in the art such as, but not limited to, protein ligation.

In some embodiments, the linear RNA may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear RNA into a 5′ monophosphate may occur by a two-step reaction comprising: (a) contacting the 5′ nucleotide of the linear RNA with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, RNA may be circularized using the methods described in WO2017222911 and WO2016197121, the contents of each of which are herein incorporated by reference in their entirety.

In some embodiments, RNA may be circularized, for example, by back splicing of a non-mammalian exogenous intron or splint ligation of the 5′ and 3′ ends of a linear RNA. In one embodiment, the circular RNA is produced from a recombinant nucleic acid encoding the target RNA to be made circular. As a non-limiting example, the method comprises: a) producing a recombinant nucleic acid encoding the target RNA to be made circular, wherein the recombinant nucleic acid comprises in 5′ to 3′ order: i) a 3′ portion of an exogenous intron comprising a 3′ splice site, ii) a nucleic acid sequence encoding the target RNA, and iii) a 5′ portion of an exogenous intron comprising a 5′ splice site; b) performing transcription, whereby RNA is produced from the recombinant nucleic acid; and c) performing splicing of the RNA, whereby the RNA circularizes to produce a oRNA.

While not wishing to be bound by theory, circular RNAs generated with exogenous introns are recognized by the immune system as “non-self” and trigger an innate immune response. On the other hand, circular RNAs generated with endogenous introns are recognized by the immune system as “self” and generally do not provoke an innate immune response, even if carrying an exon comprising foreign RNA.

Accordingly, circular RNAs can be generated with either an endogenous or exogenous intron to control immunological self/non-self discrimination as desired. Numerous intron sequences from a wide variety of organisms and viruses are known and include sequences derived from genes encoding proteins, ribosomal RNA (rRNA), or transfer RNA (tRNA).

Circular RNAs can be produced from linear RNAs in a number of ways. In some embodiments, circular RNAs are produced from a linear RNA by backsplicing of a downstream 5′ splice site (splice donor) to an upstream 3′ splice site (splice acceptor). Circular RNAs can be generated in this manner by any nonmammalian splicing method. For example, linear RNAs containing various types of introns, including self-splicing group I introns, self-splicing group II introns, spliceosomal introns, and tRNA introns can be circularized. In particular, group I and group II introns have the advantage that they can be readily used for production of circular RNAs in vitro as well as in vivo because of their ability to undergo self-splicing due to their autocatalytic ribozyme activity.

In some embodiments, circular RNAs can be produced in vitro from a linear RNA by chemical or enzymatic ligation of the 5′ and 3′ ends of the RNA. Chemical ligation can be performed, for example, using cyanogen bromide (BrCN) or ethyl-3-(3′-dimethylaminopropyl) carbodiimide (EDC) for activation of a nucleotide phosphomonoester group to allow phosphodiester bond formation. See e.g., Sokolova (1988) FEBS Lett 232:153-155; Dolinnaya et al. (1991) Nucleic Acids Res., 19:3067-3072; Fedorova (1996) Nucleosides Nucleotides Nucleic Acids 15:1 137-1 147; herein incorporated by reference. Alternatively, enzymatic ligation can be used to circularize RNA. Exemplary ligases that can be used include T4 DNA ligase (T4 Dnl), T4 RNA ligase 1 (T4 Rnl 1), and T4 RNA ligase 2 (T4 Rnl 2).

In some embodiments, splint ligation using an oligonucleotide splint that hybridizes with the two ends of a linear RNA can be used to bring the ends of the linear RNA together for ligation. Hybridization of the splint, which can be either a DNA or a RNA, orientates the 5′-phosphate and 3′-OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, in some cases is more efficient than enzymatic ligation if the structure of the hybridized splint-RNA complex interferes with enzymatic activity.

In some embodiments, the oRNA may further comprise an internal ribosome entry site (IRES) operably linked to an RNA sequence encoding a polypeptide. Inclusion of an IRES permits the translation of one or more open reading frames from a circular RNA. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques 1997 22 150-161).

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

### Splicing Element

In some embodiments, the oRNA includes at least one splicing element. The splicing element can be a complete splicing element that can mediate splicing of the oRNA or the spicing element can be a residual splicing element from a completed splicing event. For instance, in some cases, a splicing element of a linear RNA can mediate a splicing event that results in circularization of the linear RNA, thereby the resultant oRNA comprises a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the oRNA includes a splicing element adjacent each expression sequence. In some embodiments, the splicing element is on one or both sides of each expression sequence, leading to separation of the expression products, e.g., peptide(s) and or polypeptide(s).

In some embodiments, the oRNA includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the oRNA includes at least one repetitive nucleotide sequence described elsewhere herein as an internal splicing element. In such embodiments, the repetitive nucleotide sequence may include repeated sequences from the Alu family of introns. Sec, e.g., U.S. Pat. No. 11,058,706.

In some embodiments, the oRNA may include canonical splice sites that flank head-to-tail junctions of the oRNA.

In some embodiments, the oRNA may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′,3′-cyclic phosphate. Circularization proceeds by nucleophilic attack of the 5′-OH group onto the 2′,3′-cyclic phosphate of the same molecule forming a 3′,5′-phosphodiester bridge.

In some embodiments, the oRNA may include a sequence that mediates self-ligation. Non-limiting examples of sequences that can mediate self-ligation include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear RNA may include complementary sequences, including either repetitive or nonrepetitive nucleic acid sequences within individual introns or across flanking introns. In some embodiments, the oRNA includes a repetitive nucleic acid sequence. In some embodiments, the repetitive nucleotide sequence includes poly CA or poly UG sequences. In some embodiments, the oRNA includes at least one repetitive nucleic acid sequence that hybridizes to a complementary repetitive nucleic acid sequence in another segment of the oRNA, with the hybridized segment forming an internal double strand. In some embodiments, repetitive nucleic acid sequences and complementary repetitive nucleic acid sequences from two separate oRNA that hybridize to generate a single oRNA, with the hybridized segments forming internal double strands. In some embodiments, the complementary sequences are found at the 5′ and 3′ ends of the linear RNA. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the oRNA. Such methods may include, but are not limited to click chemistry (e.g., alkyne- and azide-based methods, or clickable bases), olefin metathesis, phosphoramidate ligation, hemiaminal-imine crosslinking, base modification, and any combination thereof. In some embodiments, enzymatic methods of circularization may be used to generate the oRNA. In some embodiments, a ligation enzyme, e.g., DNA or RNA ligase, may be used to generate a template of the oRNA or complement, a complementary strand of the oRNA, or the oRNA. Any of the circular polynucleotides as taught in for example U.S. Provisional Application No. 61/873,010 filed Sep. 3, 2013 or U.S. Pat. No. 10,709,779, may be used herein. The contents of these references are incorporated herein by reference in their entirety. In addition, any of the circular RNAs, methods for making circular RNAs, circular RNA compositions that are described in the following publications are contemplated herein and are incorporated by reference in their entireties are part of the instant specification: U.S. Pat. Nos. 11,352,640, 11,352,641, 11,203,767, 10,683,498, 5,773,244, and 5,766,903; US Application Publications US 2022/0177540, US 2021/0371494, US 2022/0090137, US 2019/0345503, and US 2015/0299702; and PCT Application Publications WO 2021/226597, WO 2019/236673, WO 2017/222911, WO2016/187583, WO2014/082644, WO 1997/007825, and WO 2023/182948.

Reference is also made to the following scientific publications that provide information and details on the production of circular RNA molecules: (1) Chen X, Lu Y. Circular RNA: Biosynthesis in vitro. Front Bioeng Biotechnol. 2021 Nov. 30; 9:787881. doi: 10.3389/fbioe.2021.787881. PMID: 34917603; PMCID: PMC8670002; (2) Lee K H, Kim S, Lee S W. Pros and Cons of In Vitro Methods for Circular RNA Preparation. Int J Mol Sci. 2022 Oct. 31; 23(21):13247. doi: 10.3390/ijms232113247. PMID: 36362032; PMCID: PMC9654983; (3) Liu X, Zhang Y, Zhou S, Dain L, Mei L, Zhu G. Circular RNA: An emerging frontier in RNA therapeutic targets, RNA therapeutics, and mRNA vaccines. J Control Release. 2022 August; 348:84-94. doi: 10.1016/j.jconrel.2022.05.043. Epub 2022 Jun. 2. PMID: 35649485; PMCID: PMC9644292; (4) Müller S, Appel B. In vitro circularization of RNA. RNA Biol. 2017 Aug. 3; 14(8):1018-1027. doi: 10.1080/15476286.2016.1239009. Epub 2016 Sep. 26. PMID: 27668458; PMCID: PMC5680678; (5) Petkovic S, Müller S. Synthesis and Engineering of Circular RNAs. Methods Mol Biol. 2018; 1724:167-180. doi: 10.1007/978-1-4939-7562-4_14. PMID: 29322449; (6) Welden J R, Stamm S. Pre-mRNA structures forming circular RNAs. Biochim Biophys Acta Gene Regul Mech. 2019 November-December; 1862(11-12):194410. doi: 10.1016/j.bbagrm.2019.194410. Epub 2019 Aug. 14. PMID: 31421281; and (7) Prats A C, David F, Diallo L H, Roussel E, Tatin F, Garmy-Susini B, Lacazette E. Circular RNA, the Key for Translation. Int J Mol Sci. 2020 Nov. 14; 21(22):8591. doi: 10.3390/ijms21228591. PMID: 33202605; PMCID: PMC7697609; each of which are incorporated herein by reference in their entireties.

### E. Gene Editing Systems

As described herein in various embodiments, the LNPs of the present disclosure comprise a gene editing system. As used herein, the term “gene editing system” (used interchangeably herein with the term “nucleobase editing system”) generally refers to a composition having one or more gene editing system components which are capable of independently or co-dependently editing, modifying, or altering a target polynucleotide sequence or a target transcript comprising a nucleic acid sequence and/or modifying the epigenome to effect a change in gene regulation. In certain embodiments, gene editing systems of the present disclosure include any editing systems known in the art. Gene editing systems, nucleobase editing systems, nucleobase editing tools and similar terms throughout should be understood to refer to interchangeable concepts. As described herein, all references to LNPs comprising gene editing systems should be understood to also contemplate LNPs comprising polynucleotides encoding said gene editing systems.

For example, the LNP compositions herein may be used to deliver any gene editing system including CRISPR (clustered regularly interspaced short palindromic repeats) and the associated CRISPR-associated proteins (e.g., CRISPR-Cas9) (Jinek et al., “A programmable dual-RNA-guided DNA endonuclease in adaptive bacterial immunity,” Science, Vol. 337 (6096), pp. 816-821), meganuclease editors (Boissel et al., “megaTALs: a rare-cleaving nuclease architecture for therapeutic genome engineering,” Nucleic Acids Research 42: pp. 2591-2601) and bacterial retron systems (Schubert et al., “High-throughput functional variant screens via in vivo production of single-stranded DNA,” PNAS, Apr. 27, 2021, Vol. 118(18), pp. 1-10). In particular, CRISPR-Cas9 has been derivatized in numerous ways to expand upon its guide RNA-based programmable double-strand cutting activity to form systems ranging from finding alternative CRISPR Cas nuclease enzymes having different PAM requirements and cutting properties (e.g., Cas12a, Cas12f, Cas13a, and Cas13b) to base editing (Komor et al., “Programmable editing of a target base in genomic DNA without double-stranded DNA cleavage,” Nature, May 19, 2016, 533(7603); pp. 420-424 [cytosine base editors or CBEs] and Gaudelli et al., “Programmable base editing of A-T to G-C in genomic DNA without DNA cleavage,” Nature, Vol. 551, pp. 464-471 [adenine base editors or ABEs]) to prime editing (Anzalone et al., “Search-and-replace genome editing without double-strand breaks or donor DNA,” Nature, December 2019, 576(7789): pp. 149-157) to twin prime editing (Anzalone et al., “Programmable deletion, replacement, integration and inversion of large DNA sequences with twin prime editing,” Nature Biotechnology, Dec. 9, 2021, vol. 40, pp. 731-740) to epigenetic editing (Kungulovski and Jeltsch, “Epigenome Editing: State of the Art, Concepts, and Perspective,” Trends in Genetics, Vol. 32, 206, pp. 101-113) to CRISPR-directed integrase editing (Yarnell et al., “Drag-and-drop genome insertion of large sequences without double-stranded DNA cleavage using CRISPR-directed integrases,” Nature Biotechnology, Nov. 24, 2022, (“PASTE”). Each of these gene editing systems may be packaged up in the LNP compositions described herein and delivered to target organs, tissues, and cells to bring about the modification of a target sequence or the expression of a target gene.

The gene editing systems deliverable by the herein disclosed LNPs can be any gene editing system. In certain embodiments, the LNPs of the present disclosure are used to delivery gene editing systems capable of editing, modifying or altering a target polynucleotide sequence that results in treatment of a hemoglobinopathy. In still other embodiments, the gene editing systems are preferably, but not limited to, those disclosed herein. The gene editing systems contemplated herein can include (A) nucleobase gene editing systems which result in one or more the modifications to the sequence of target nucleic acid molecule, (B) an epigenetic editing system which results in one or more modifications to the epigenome to bring about an effect on gene expression without altering the sequence of a nucleic acid molecule, and (C) gene editing systems that combine the features of nucleobase editing systems and epigenetic editing systems.

Nucleobase editing systems include a wide array of configurations with various combinations of protein functionalities and/or nucleic acid molecule components, all of which are contemplated herein. In general, nucleobase editing systems comprise at least a (i) DNA binding domain that is user-programmable to target a specific sequence in a nucleic acid molecule and optionally (ii) one or more effector domains that facilitate the modification of the sequence of the nucleic acid molecule. User-programmability may comprise amino acid sequence-programmable DNA binding domains (e.g., TALENS and zinc finger-binding domains) or nucleic acid sequence-programmable DNA binding domains (e.g., CRISPR Cas9, CRISPR Cas12a, CRISPR Cas12f, CRISPR Cas13a, CRISPR Cas13b, IscB, IsrB, or TnpB). Similarly, epigenetic editing systems comprise at least a (i) DNA binding domain that targets a specific sequence in a nucleic acid molecule and (ii) one or more effector domains that facilitates the modification of one or more epigenomic features of the nucleic acid molecule.

In certain embodiments, gene editing systems further comprise one or more effector domains that provide various functionalities that facilitate changes in nucleotide sequence and/or gene expression, such as, but not limited to, single-strand DNA binding proteins, nucleases, endonucleases, exonucleases, deaminases (e.g., cytidine deaminases or adenosine deaminases), polymerases (e.g., reverse transcriptases), integrases, recombinases, etc., and fusion proteins comprising one or more functional domains linked together). In certain embodiments, the nucleobase editing systems include, but are not limited to, systems comprising a clustered regularly interspaced short palindromic repeats (“CRISPR”)-associated (“Cas”) protein, a zinc finger nuclease (“ZFN”), a transcription activator-like effector nuclease (“TALEN”), an adenosine deaminase acting on RNA (“ADAR”) enzyme, an adenosine deaminase acting on transfer RNA (“ADAT”) enzyme, an activation induced cytidine deaminase (“AID”)/apolipoprotein B editing complex (“APOBEC”) enzyme, a meganuclease, IscB, IsrB, TnpB, or a restriction enzyme.

In some embodiments, the nucleobase editing system edits, modifies, or alters the target polynucleotide sequence ex vivo. In some embodiments, the nucleobase editing system edits, modifies, or alters the target polynucleotide sequence in vivo. In some embodiments, the nucleobase editing system edits, modifies, or alters the target polynucleotide sequence in a cell. In some embodiments, the cell is a mammalian cell. In some embodiments, the cell is a human cell.

In some embodiments, the target polynucleotide sequence is a gene or a regulatory sequence that controls transcription of a gene (e.g., a promoter, transcription binding site, enhancer sequence, etc.) or a sequence which controls the translation of a messenger RNA. In some embodiments, the target transcript comprising a nucleic acid sequence is a product of gene transcription. In some embodiments, the target transcript comprising a nucleic acid sequence is an RNA transcript such as a messenger RNA transcript, microRNA transcript or transfer RNA transcript.

The originator constructs and benchmark constructs of the present disclosure may comprise, encode or be conjugated to a cargo which is a nucleobase editing tool. As used herein, the term “nucleobase editing tool” is used interchangeably with “nucleobase editing system component” and generally refers to a compound or substance which is capable of independently or co-dependently editing, modifying, or altering a target polynucleotide sequence or a target transcript comprising a nucleic acid sequence. Nucleobase editing tools for the present disclosure include all nucleobase editing tools known in the art. In certain embodiments, the nucleobase editing tools include, but not limited to, effector proteins which modify DNA or RNA, guide elements which guide effector proteins to specific DNA or RNA sequence, repair elements which encode a nucleic acid sequence template, and supportive elements which activate or modulate the activity of another nucleobase editing tool, or activates or modulates host DNA repair enzymes.

In some embodiments, the cargo may comprise a nucleobase editing tool or a polynucleotide encoding a nucleobase editing tool. In some embodiments, the cargo may comprise one or more polynucleotides encoding a nucleobase editing tool. In some embodiments, the cargo may comprise a polynucleotide encoding one or more nucleobase editing tools. In some embodiments, the cargo may comprise a polynucleotide that is a component of the nucleobase editing tool. In some embodiments, the cargo may comprise a polynucleotide encoding one or more protein or peptide components in the nucleobase editing tool.

In some embodiments, the cargo may comprise an effector protein capable of modifying a target DNA or RNA sequence. In some embodiments, the cargo may comprise a polynucleotide encoding an effector protein. In certain embodiments, the effector proteins include polymerases, nucleases, mutator enzymes, reverse transcriptases, recombinases, integrases, endonucleases, exonucleases, transposases, and deaminases. As used herein, the term “polymerases,” includes enzymes which catalyze the synthesis of DNA or RNA polymers. As used herein, the term “nucleases,” includes enzymes which cleave nucleobases. In certain embodiments, nucleases include enzymes which create single-stranded breaks (“SSB”) or double-stranded breaks (“DSB”) in nucleic acid sequences. As used herein, the term “mutator enzymes,” in its broadest sense, includes enzymes which mutate nucleic acid sequences. In certain embodiments, the cargo may comprise nucleases such as effector proteins include clustered regularly interspaced short palindromic repeats (“CRISPR”)-associated (“Cas”) proteins, zinc finger nucleases (“ZFNs”), transcription activator-like effector nucleases (“TALENs”), adenosine deaminase acting on RNA (“ADAR”) enzymes, adenosine deaminase acting on transfer RNA (“ADAT”) enzymes, activation induced cytidine deaminase (“AID”)/apolipoprotein B editing complex (“APOBEC”) enzymes, meganucleases, IscB, IsrB, TnpB, or restriction enzymes.

In some embodiments, the cargo may comprise a guide element which guide effector proteins to target a DNA or RNA sequence. In some embodiments, the cargo may comprise a polynucleotide encoding a guide element. In certain embodiments, guide elements include guide RNAs (“gRNAs”), CRISPR RNAs (“crRNAs”), prime editing guide RNAs (“pegRNAs”), transcription activator-like effectors (TALEs), or antisense oligomers.

In some embodiments, the cargo may further comprise a repair element which encodes a sequence repair template. In some embodiments, the cargo may further comprise a polynucleotide encoding a repair element or sequence repair template.

In some embodiments, the cargo may further comprise a supportive element which activates or modulates the editing system. In some embodiments, the cargo may further comprise a supportive element which activates or modulates the effector protein. In some embodiments, the cargo may further comprise a polynucleotide encoding a supportive element. Non-limiting categories of supportive elements include trans-activating RNA (“tracrRNA”).

### CRISPR-Cas Editors

In some embodiments, the LNPs may be used to deliver a CRISPR-Cas gene editing system comprising a CRISPR-Cas programmable nuclease, such as a CRISPR-type II nuclease (e.g., Cas9) or CRISPR-type V nuclease (e.g., Cas12a) or a homolog or variant thereof, and a suitable guide RNA. The programmable nuclease and the guide RNA form a complex. The complex then scans a target DNA for an appropriate PAM sequence (protospacer adjacent motif) recognized by the programmable nuclease and a protospacer sequence in one strand of the DNA which matches the spacer sequence of the guide RNA. The guide RNA spacer sequence then hybridizes to the sequence on the opposite strand that is complementary to the protospacer sequence (the target sequence since it the sequence that interacts directly with the spacer of the guide RNA).

In general, nucleobase editing systems comprise at least a (i) DNA binding domain that is user-programmable to target a specific sequence in a nucleic acid molecule and optionally (ii) one or more effector domains that facilitate the modification of the sequence of the nucleic acid molecule. User-programmability may comprise amino acid sequence-programmable DNA binding domains (e.g., TALENS and zinc finger-binding domains) or nucleic acid sequence-programmable DNA binding domains (e.g., CRISPR Cas9, CRISPR Cas12a, CRISPR Cas12f, CRISPR Cas13a, CRISPR Cas13b, or TnpB), and including a guide RNA which targets the programmable DNA binding protein to target sequence.

In some embodiments, the CRISPR-Cas system comprises a Cas or Cas-derived protein.

In other embodiments, the sequence-programmable DNA binding domains (e.g., RNA-guided nuclease) used for genome modification is a clustered regularly interspersed short palindromic repeats (CRISPR) system Cas nuclease. Any RNA-guided Cas nuclease capable of catalyzing site-directed cleavage of DNA to allow integration of donor polynucleotides by the HDR mechanism can be used in genome editing, including CRISPR system Class 1, Type I, II, or III Cas nucleases; Class 2, Type II nuclease (such as Cas9); a Class 2, Type V nuclease (such as Cpf1), or a Class 2, Type VI nuclease (such as C2c2). Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), CasIO, Cas1Od, CasF, CasG, CasH, Csy1, Csy2, Csy3, Csc1 (CasA), Csc2 (CasB), Csc3 (CasE), Csc4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cu1966, and homologs or modified versions thereof. In some embodiments, an LNP of the present disclosure comprises gene editing system is or comprises a Type V nuclease editing system described in International Application Publication WO2024020346A1, which is incorporated by reference herein in its entirety.

In some embodiments, the CRISPR-Cas gene editing system can be a CRISPR-type II gene editing system. As defined herein, the term “CRISPR-type II gene editing system” refers to a set of components capable of conducting nucleic acid programmable gene editing that minimally comprises a CRISPR type II nucleic acid programmable nuclease (e.g., Cas9 or homolog or variant thereof, e.g., a dead Cas9 or Cas9 nickase) and a guide RNA which complexes with said nuclease. The guide RNA is capable of complexing with a CRISPR type II nuclease and directing the complex to a target DNA site that is complementary to the spacer sequence of the guide. This term also embraces systems comprising additional accessory proteins, such as another nuclease, reverse transcriptase, deaminase, recombinase, transposase, integrase, DNA binding protein, transcription factor, a CRISPR Cas protein, or an anti-CRISPR inhibitor protein. In one embodiment, a CRISPR-type II gene editing system can be a base editor comprising CRISPR type II nucleic acid programmable nuclease (e.g., Cas9 or homolog or variant thereof, e.g., a dead Cas9 or Cas9 nickase), a guide RNA, and one or more deaminases, wherein the one or more deaminases is optionally fused to the nuclease via a linker. In another embodiment, a CRISPR-type II gene editing system can be a prime editor comprising CRISPR type II nucleic acid programmable nuclease (e.g., Cas9 or homolog or variant thereof, e.g., a dead Cas9 or Cas9 nickase), a prime editing guide RNA (pegRNA), and a reverse transcriptase, wherein the reverse transcriptase is optionally fused to the nuclease via a linker. In various embodiments, the CRISPR-type II gene editing system components, such as the nuclease, guide RNAs, or accessory proteins, may comprise one or more NLSs to facilitate their transfer into the nucleus. The gene editing system may be in the form of DNA (encoding the protein components and the guide RNA), RNA (encoding the protein components and constituting a guide RNA), or a mixture of protein components (e.g., a nuclease) and RNA (e.g., a guide RNA).

In other embodiments, the CRISPR-Cas gene editing system can be a CRISPR-type V gene editing system. As defined herein the term “CRISPR-type V gene editing system” refers to a set of components capable of conducting nucleic acid programmable gene editing that minimally comprises a CRISPR type V nucleic acid programmable nuclease (e.g., Cas12a or homolog or variant, e.g., a dead Cas12a or Cas12a nickase) and a guide RNA which complexes with said nuclease. The guide RNA is capable of complexing with a CRISPR type V nuclease and directing the complex to a target DNA site that is complementary to the spacer sequence of the guide. In one embodiment, a CRISPR-type V gene editing system can be a base editor comprising CRISPR type V nucleic acid programmable nuclease (e.g., Cas12a or homolog or variant thereof, e.g., a dead Cas12a or Cas12a nickase), a guide RNA, and one or more deaminases, wherein the one or more deaminases is optionally fused to the nuclease via a linker. In another embodiment, a CRISPR-type V gene editing system can be a prime editor comprising CRISPR type V nucleic acid programmable nuclease (e.g., Cas12a or homolog or variant thereof, e.g., a dead Cas12a or Cas12a nickase), a prime editing guide RNA (pegRNA), and a reverse transcriptase, wherein the reverse transcriptase is optionally fused to the nuclease via a linker. In various embodiments, the CRISPR-type V gene editing system components, such as the nuclease, guide RNAs, or accessory proteins, may comprise one or more NLSs to facilitate their transfer into the nucleus. The gene editing system may be in the form of DNA (encoding the protein components and the guide RNA), RNA (encoding the protein components and constituting a guide RNA), or a mixture of protein components (e.g., a nuclease) and RNA (e.g., a guide RNA). In various embodiments, the type V nuclease may comprise any amino acid sequence

In some embodiments, a Class 1, type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP 002989955, WP_038434062, WP_011528583); Campylobacter jejuni (WP_022552435, YP 002344900), Campylobacter coli (WP 060786116); Campylobacter fetus (WP 059434633); Corynebacterium ulcerans (NC_015683, NC_017317); Corynebacterium diphtheria (NC_016782, NC_016786); Enterococcus faecalis (WP 033919308); Spiroplasma syrphidicola (NC 021284); Prevotella intermedia (NC 017861); Spiroplasma taiwanense (NC 021846); Streptococcus iniae (NC 021314); Belliella baltica (NC 018010); Psychroflexus torquisl (NC O 18721); Streptococcus thermophilus (YP 820832), Streptococcus mutans (WP 061046374, WP 024786433); Listeria innocua (NP 472073); Listeria monocytogenes (WP 061665472); Legionella pneumophila (WP 062726656); Staphylococcus aureus (WP_001573634); Francisella tularensis (WP_032729892, WP_014548420), Enterococcus faecalis (WP 033919308); Lactobacillus rhamnosus (WP 048482595, WP_032965177); and Neisseria meningitidis (WP_061704949, YP_002342100); all of which sequences (as entered by the date of filing of this application) are herein incorporated by reference in their entireties. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacterid. 198(5):797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

In another embodiment, the gene editing system delivered by the LNP-based RNA medicines described herein may comprise a CRISPR-Cas12a (Cpf1) nuclease. Cpf1 was first identified from Prevotella and Francisella 1 (Cpf1, or Cas12a) and published in Zetsche et al., “Cpf1 is a single RNA-guided endonuclease of a class 2 CRISPR-Cas system,” Cell, Oct. 22, 2015, 163, pp. 759-711, which is incorporated herein by reference. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163(3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

The gene editing systems described herein can include any known Cas12a nuclease, or any variant thereof, such as any Cas12a ortholog described in U.S. patent application Ser. No. 18/297,346, U.S. patent application Ser. No. 18/481,393, or Intern International Application Publication WO2024020346A1, or an amino acid sequence having at least 75%, 80%, 85%, 90%, 95%, 99%, or up to 100% sequence identity with any of the Cas12a orthologs described in said patent applications.

The gene editing systems described herein can include any known Cas12a nuclease, or any variant thereof, such as any Cas12a ortholog described in (1) Wu J, Gao P, Shi Y, Zhang C, Tong X, Fan H, Zhou X, Zhang Y, Yin H. Characterization of a thermostable Cas12a ortholog. Cell Insight. 2023 Oct. 11; 2(6):100126. doi: 10.1016/j.cellin.2023.100126. PMID: 38047138; PMCID: PMC10692460; (2) Swarts D C, Jinek M. Cas9 versus Cas12a/Cpf1: Structure-function comparisons and implications for genome editing. Wiley Interdiscip Rev RNA. 2018 September; 9(5):e1481. doi: 10.1002/wrna.1481. Epub 2018 May 22. PMID: 29790280; (3) Kleinstiver B P, Sousa A A, Walton R T, Tak Y E, Hsu J Y, Clement K, Welch M M, Horng J E, Malagon-Lopez J, Scarfó I, Maus M V, Pinello L, Aryee M J, Joung J K. Engineered CRISPR-Cas12a variants with increased activities and improved targeting ranges for gene, epigenetic and base editing. Nat Biotechnol. 2019 March; 37(3):276-282. doi: 10.1038/s41587-018-0011-0. Epub 2019 Feb. 11. Erratum in: Nat Biotechnol. 2020 July; 38(7):901. PMID: 30742127; PMCID: PMC6401248; (4) Ma E, Chen K, Shi H, Stahl E C, Adler B, Trinidad M, Liu J, Zhou K, Ye J, Doudna J A. Improved genome editing by an engineered CRISPR-Cas12a. Nucleic Acids Res. 2022 Dec. 9; 50(22):12689-12701. doi: 10.1093/nar/gkac1192. PMID: 36537251; PMCID: PMC9825149; (5) Li T, Zhu L, Xiao B, Gong Z, Liao Q, Guo J. CRISPR-Cpf1-mediated genome editing and gene regulation in human cells. Biotechnol Adv. 2019 January-February; 37(1):21-27. doi: 10.1016/j.biotechadv.2018.10.013. Epub 2018 Nov. 3. PMID: 30399413 (6); Jacobsen T, Liao C, Beisel C L. The Acidaminococcus sp. Cas12a nuclease recognizes GTTV and GCTV as non-canonical PAMs. FEMS Microbiol Lett. 2019 Apr. 1; 366(8):fnz085. doi: 10.1093/femsle/fnz085. PMID: 31004485; PMCID: PMC6604746; (7) Huang H, Huang G, Tan Z, Hu Y, Shan L, Zhou J, Zhang X, Ma S, Lv W, Huang T, Liu Y, Wang D, Zhao X, Lin Y, Rong Z. Engineered Cas12a-Plus nuclease enables gene editing with enhanced activity and specificity. BMC Biol. 2022 Apr. 25; 20(1):91. doi: 10.1186/s12915-022-01296-1. PMID: 35468792; PMCID: PMC9040236; (8) Zhu D, Wang J, Yang D, Xi J, Li J. High-Throughput Profiling of Cas12a Orthologues and Engineered Variants for Enhanced Genome Editing Activity. Int J Mol Sci. 2021 Dec. 10; 22(24):13301. doi: 10.3390/ijms222413301. PMID: 34948095; PMCID: PMC8706968; (9) Gao L, Cox D B T, Yan W X, Manteiga J C, Schneider M W, Yamano T, Nishimasu H, Nureki O, Crosetto N, Zhang F. Engineered Cpf1 variants with altered PAM specificities. Nat Biotechnol. 2017 August; 35(8):789-792. doi: 10.1038/nbt.3900. Epub 2017 Jun. 5. PMID: 28581492; PMCID: PMC5548640; (10) Zhang L, Zuris J A, Viswanathan R, Edelstein J N, Turk R, Thommandru B, Rube H T, Glenn S E, Collingwood M A, Bode N M, Beaudoin S F, Lele S, Scott S N, Wasko K M, Sexton S, Borges C M, Schubert M S, Kurgan G L, McNeill M S, Fernandez C A, Myer V E, Morgan R A, Behlke M A, Vakulskas C A. AsCas12a ultra nuclease facilitates the rapid generation of therapeutic cell medicines. Nat Commun. 2021 Jun. 23; 12(1):3908. doi: 10.1038/s41467-021-24017-8. Erratum in: Nat Commun. 2021 Jul. 19; 12(1):4500. PMID: 34162850; PMCID: PMC8222333; (11) Ling X, Chang L, Chen H, Gao X, Yin J, Zuo Y, Huang Y, Zhang B, Hu J, Liu T. Improving the efficiency of CRISPR-Cas12a-based genome editing with site-specific covalent Cas12a-crRNA conjugates. Mol Cell. 2021 Nov. 18; 81(22):4747-4756.e7. doi: 10.1016/j.molcel.2021.09.021. Epub 2021 Oct. 13. PMID: 34648747; and (12) Zhou J, Chen P, Wang H, Liu H, Li Y, Zhang Y, Wu Y, Paek C, Sun Z, Lci J, Yin L. Cas12a variants designed for lower genome-wide off-target effect through stringent PAM recognition. Mol Ther. 2022 Jan. 5; 30(1):244-255. doi: 10.1016/j.ymthe.2021.10.010. Epub 2021 Oct. 20. PMID: 34687846; PMCID: PMC8753454; each of which are incorporated by reference in their entireties.

Any publicly known Cas12a/Cpf1 may be used as a component of the gene editing systems described herein, including but not limited to, the following publicly available amino acid sequences: GenBank Accession No. QOE76068.1; GenBank Accession No. WKU83685.1; GenBank Accession No. WBC51234.1; GenBank Accession No. QOL02411.1; GenBank Accession No. UVJ64960.1; GenBank Accession No. UVJ64958.1; GenBank Accession No. UVJ64957.1; GenBank Accession No. UVJ64956.1; GenBank Accession No. UVJ64954.1; GenBank Accession No. UVJ64953.1; GenBank Accession No. UVJ64952.1; GenBank Accession No. UVJ64951.1; GenBank Accession No. UVJ64949.1; GenBank Accession No. UVJ64947.1; GenBank Accession No. UVJ64946.1; GenBank Accession No. UVJ64945.1; GenBank Accession No. WP_320869194.1; GenBank Accession No. WOZ85592.1; GenBank Accession No. WOF96262.1; and any amino acid sequence having at least 75%, 80%, 85%, 90%, 95%, 99%, or up to 100% sequence identity with any of the aforementioned sequence, or any other publicly available Cas12a sequence.

C2c1 (Cas12b) is another class II CRISPR/Cas system RNA-guided nuclease that may be used. C2c1, similarly to Cas9, depends on both a crRNA and tracrRNA for guidance to target sites. See, e.g., Shmakov et al. (2015) Mol Cell. 60(3):385-397, Zhang et al. (2017) Front Plant Sci. 8:177; herein incorporated by reference.

In one aspect, a nucleic acid sequence-programmable DNA binding domain can be associated with or complexed with at least one guide nucleic acid (e.g., guide RNA or a pegRNA), which localizes the DNA binding domain to a DNA sequence that comprises a DNA strand (i.e., a target strand) that is complementary to the guide nucleic acid, or a portion thereof (e.g., the spacer of a guide RNA which anneals to the protospacer of the DNA target). In other words, the guide nucleic-acid “programs” the DNA binding domain (e.g., Cas9 or equivalent) to localize and bind to complementary sequence of the protospacer in the DNA.

Any suitable nucleic acid sequence-programmable DNA binding domain may be used in the prime editors described herein. In various embodiments, the nucleic acid sequence-programmable DNA binding domain may be any Class 2 CRISPR-Cas system, including any type II, type V, or type VI CRISPR-Cas enzyme. Given the rapid development of CRISPR-Cas as a tool for genome editing, there have been constant developments in the nomenclature used to describe and/or identify CRISPR-Cas enzymes, such as Cas9 and Cas9 orthologs. CRISPR-Cas nomenclature is extensively discussed in Makarova et al., “Classification and Nomenclature of CRISPR-Cas Systems: Where from Here?,” The CRISPR Journal, Vol. 1. No. 5, 2018, the entire contents of which are incorporated herein by reference.

Without being bound by theory, the mechanism of action of certain CRISPR Cas enzymes contemplated herein includes the step of forming an R-loop whereby the Cas protein induces the unwinding of a double-strand DNA target, thereby separating the strands in the region bound by the Cas protein. The guide RNA spacer then hybridizes to the “target strand” at a region that is complementary to the protospacer sequence of the DNA. In some embodiments, the Cas protein may include one or more nuclease activities, which then cut the DNA leaving various types of lesions. For example, the Cas protein may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary Cas proteins with different nuclease activities include “Cas9 nickase” (“nCas9”) and a deactivated Cas9 having no nuclease activities (“dead Cas9” or “dCas9”).

The below description of various Cas proteins which can be used in connection with the presently disclosed LNP-delivered gene editing systems is not meant to be limiting in any way. The gene editing systems may comprise the canonical SpCas9 or Cas12a, or any ortholog Cas9 protein or Cas12a protein, or any variant Cas9 or Cas12a protein—including any naturally occurring variant, mutant, or otherwise engineered version of Cas9—that is known or which can be made or evolved through a directed evolutionary or otherwise mutagenic process. In various embodiments, the Cas9 or Cas9 variants have a nickase activity, i.e., only cleave one strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure.

The gene editing systems described herein may also comprise Cas9 equivalents, including Cas12a (Cpf1) and Cas12b1 proteins. The Cas proteins usable herein (e.g., SpCas9, Cas9 variant, or Cas9 equivalents) may also contain various modifications that alter/enhance their PAM specificities. The present disclosure contemplates any Cas9, Cas9 variant, or Cas9 equivalent which has at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity to a reference Cas9 sequence, such as a reference SpCas9 canonical sequence of Streptococcus pyogenes M1 (Accession No. Q99ZW2).

The Cas proteins contemplated herein embrace CRISPR Cas 9 proteins, as well as Cas9 equivalents, variants (e.g., Cas9 nickase (nCas9) or nuclease inactive Cas9 (dCas9)) homologs, orthologs, or paralogs, whether naturally occurring or non-naturally occurring (e.g., engineered or recombinant), and may include a Cas9 equivalent from any Class 2 CRISPR system (e.g., type II, V, VI), including Cas12a (Cpf1), Cas12e (CasX), Cas12b1 (C2c1), Cas12b2, Cas12c (C2c3), C2c4, C2c8, C2c5, C2c10, C2c9 Cas13a (C2c2), Cas13d, Cas13c (C2c7), Cas13b (C2c6), and Cas13b. Further Cas-equivalents are described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018, the contents of which are incorporated herein by reference.

The terms “Cas9” or “Cas9 nuclease” or “Cas9 moiety” or “Cas9 domain” embrace any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. The term Cas9 is not meant to be particularly limiting and may be referred to as a “Cas9 or equivalent.” Exemplary Cas9 proteins are further described in the art and are incorporated herein by reference. As noted herein, Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

In certain embodiments, a polynucleotide programmable nucleotide binding domain of a nucleobase editor itself comprises one or more domains. In one embodiment, a polynucleotide programmable nucleotide binding domain comprises one or more nuclease domains. In some embodiments, the nuclease domain of a polynucleotide programmable nucleotide binding domain comprises an endonuclease or an exonuclease. In some embodiments, the endonuclease cleaves a single strand of a double-stranded nucleobase. In some embodiments, the endonuclease cleaves both strands of a double-stranded nucleobase molecule. In some embodiments, the polynucleotide programmable nucleotide binding domain is a deoxyribonuclease. In some embodiments, the polynucleotide programmable nucleotide binding domain is a ribonuclease.

In some embodiments, the nuclease domain of a polynucleotide programmable nucleotide binding domain can cut zero, one, or two strands of a target polynucleotide. In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleobase molecule (e.g., DNA). In some embodiments, the nickase is derived from a fully catalytically active (e.g., natural) form of a polynucleotide programmable nucleotide binding domain by introducing one or more mutations into the active polynucleotide programmable nucleotide binding domain. In certain embodiments, where a polynucleotide programmable nucleotide binding domain comprises a nickase domain derived from Cas9. Cas12a and Cas9 nickases are known in the art and contemplated for use herein, for example as discussed in (1) Schubert M S, Thommandru B, Woodley J, Turk R, Yan S, Kurgan G, McNeill M S, Rettig G R. Optimized design parameters for CRISPR Cas9 and Cas12a homology-directed repair. Sci Rep. 2021 Sep. 30; 11(1):19482. doi: 10.1038/s41598-021-98965-y. PMID: 34593942; PMCID: PMC8484621; (2) Fu B X H, Smith J D, Fuchs R T, Mabuchi M, Curcuru J, Robb G B, Fire A Z. Target-dependent nickase activities of the CRISPR-Cas nucleases Cpf1 and Cas9. Nat Microbiol. 2019 May; 4(5):888-897. doi: 10.1038/s41564-019-0382-0. Epub 2019 Mar. 4. PMID: 30833733; PMCID: PMC6512873; (3) Xu T, Tao X, Kempher M L, Zhou J. Cas9 Nickase-Based Genome Editing in Clostridium cellulolyticum. Methods Mol Biol. 2022; 2479:227-243. doi: 10.1007/978-1-0716-2233-9_15. PMID: 35583742; (4) Wu W H, Ma X M, Huang J Q, Lai Q, Jiang F N, Zou C Y, Chen L T, Yu L. CRISPR/Cas9 (D10A) nickase-mediated Hb CS gene editing and genetically modified fibroblast identification. Bioengineered. 2022 May; 13(5):13398-13406. doi: 10.1080/21655979.2022.2069940. PMID: 36700476; PMCID: PMC9276056; each of which are incorporated herein by reference in their entireties.

In some embodiments, the Cas9-derived nickase has one or more mutations in the RuvC-1 domain. In one embodiment, the Cas9-derived nickase has a D10A mutation in the RuvC-1 domain. In some embodiments, the Cas9-derived nickase has one or more mutations in the REC Lobe domain. In one embodiment, the Cas9-derived nickase has a N497A, R661A, and/or Q695A mutation in the REC Lobe domain. In some embodiment, the Cas9-derived nickase has one or more mutations in the HNH domain. In one embodiment, the Cas9-derived nickase has H840A, N863A, and/or D839A in the HNH domain.

In certain embodiments, in the SpCas9-derived nickase, the residue H840 retains catalytic activity and can thereby cleave a single strand of the nucleobase duplex. In certain embodiments, a Cas9-derived nickase domain can comprise an H840A mutation, while the amino acid residue at position 10 remains a D. In certain embodiments, a Cas9-derived nickase domain can comprise an N863A mutation, while the amino acid residue at position 10 remains a D. In some embodiments, the nickase is derived from a fully catalytically active (e.g., natural) form of a polynucleotide programmable nucleotide binding domain by removing all or a portion of a nuclease domain that is not required for the nickase activity. In certain embodiments, where a polynucleotide programmable nucleotide binding domain comprises a nickase domain derived from Cas9, the Cas9-derived nickase domain comprises a deletion of all or a portion of the RuvC domain or the HNH domain.

In certain embodiments, the nucleobase editing system is or comprises a CRISPR-Cas editor or Cas9 disclosed and described in one or more of US Application Publications US2015/0045546A1, US2019/0264232A1, US2018/0258417A1, and PCT Publications WO2013141680A1 and WO2021173359A1, each of which is incorporated by reference herein in their entirety.

In some embodiments, the nucleobase editing system is or comprises a Cas type V-based gene editing system or any component thereof as disclosed in WO2024/020346 (PCT/US23/70339 filed Jul. 17, 2023 and published Jan. 25, 2024), the entire contents of which are incorporated herein by reference. In particular, the Cas type V-based gene editing system may comprise a Cas type V polypeptide (or a nucleic acid sequence encoding said Cas type V polypeptide) which are listed in Appendix A of WO2024/020346, or any sequence having at least 70%, 75%, 80%, 85%, 90%, 95%, 99%, or 100% sequence identity with a sequence of Appendix A, including, but not limited to the sequences designated as ID400, ID401, ID402, ID403, ID404, ID405, ID406, ID407, ID408, ID409, ID411, ID412, ID413, ID414, ID415, ID416, ID417, ID418, ID419, ID420, ID421, ID422, ID423, ID424, ID425, ID426, ID427, ID428, ID429, ID432, and ID433 (SEQ ID NOs: 436, 333, 386, 403, 387, 334, 335, 32, 401, 399, 331, 440, 118, 58, 20, 442, 336, 564, 445, 446, 119, 1, 404, 3, 447, 448, 33, 400, 59, 57, and 402 of WO2024/020346).

In other embodiments, the Cas type V-based gene editing system may comprise a Cas type V polypeptide (or a nucleic acid sequence encoding said Cas type V polypeptide) which are listed in Appendix A, Section P (mutant type V enzymes based on wildtype ID405, ID414, and ID418) of WO2024/020346, or any sequence having at least 70%, 75%, 80%, 85%, 90%, 95%, 99%, or 100% sequence identity with a sequence of Appendix A, Section P, including, but not limited to the following sequences designated as ID405-1 (which comprises a D169R substitution relative to the ID405 wildtype sequence), ID405-2 (which comprises D169R/R950K/R954A substitutions relative to the ID405 wildtype sequence), ID405-3 (which comprises D169R/N559R/Q565R substitutions relative to the ID405 wildtype sequence), ID405-4 (which comprises C554R substitution relative to the ID405 wildtype sequence), ID405-5 (which comprises C554N substitution relative to the ID405 wildtype sequence), ID405-6 (which comprises L860Q substitution relative to the ID405 wildtype sequence), ID414-1 (which comprises T154R substitution relative to the ID414 wildtype sequence), ID414-2 (which comprises T154R/R887K/R891A substitutions relative to the ID414 wildtype sequence), ID414-3 (which comprises T154R/G536R/K542R substitutions relative to the ID414 wildtype sequence), ID 414-4 (which comprises TN531R/S802L substitutions relative to the ID414 wildtype sequence), ID414-5 (which comprises N531R substitution relative to the ID414 wildtype sequence), ID414-6 (which comprises S802L substitution relative to the ID414 wildtype sequence), ID418-1 (which comprises D161R substitution relative to the ID418 wildtype sequence), ID418-2 (which comprises D161R/R888K/R892A substitutions relative to the ID418 wildtype sequence), ID418-3 (which comprises D161R/T532R/K538R substitutions relative to the ID418 wildtype sequence), ID418-4 (which comprises N527R/Q799L substitutions relative to the ID418 wildtype sequence), ID418-5 (which comprises N527R substitution relative to the ID418 wildtype sequence), ID418-6 (which comprises Q799L substitution relative to the ID418 wildtype sequence) (SEQ ID NOs: 566-602 of WO2024/020346).

Any of the above CRISPR-Cas editor embodiments or any variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

### Base Editors

In other embodiments, the LNPs may be used to deliver a base editing system. Base editors are generally composed of an engineered deaminase and a catalytically impaired CRISPR-Cas9 variant and enzymatically convert one base to another base at a specific target site with the assistance of endogenous DNA repair systems in the cell. However, base editors may also comprise Cas12a enzymes and/or other programmable nucleases. For example, Cas12a-configured base editors are described in (1) Chen F, Lian M, Ma B, Gou S, Luo X, Yang K, Shi H, Xie J, Ge W, Ouyang Z, Lai C, Li N. Zhang Q, Jin Q, Liang Y, Chen T, Wang J, Zhao X, Li L, Yu M, Ye Y, Wang K, Wa H, Lai L. Multiplexed base editing through Cas12a variant-mediated cytosine and adenine base editors. Commun Biol. 2022 Nov. 2; 5(1):1163. doi: 10.1038/s42003-022-04152-8. PMID: 36323848; PMCID: PMC9630288; (2) Wang X, Ding C, Yu W, Wang Y, He S, Yang B, Xiong Y C, Wei J, Li J, Liang J, Lu Z, Zhu W, Wu J, Zhou Z, Huang X, Liu Z, Yang L, Chen J. Cas12a Base Editors Induce Efficient and Specific Editing with Low DNA Damage Response. Cell Rep. 2020 Jun. 2; 31(9):107723. doi: 10.1016/j. celrep.2020.107723. PMID: 32492431; and (3) Swartjes T, Staals R H J, van der Oost J. Editor's cut: DNA cleavage by CRISPR RNA-guided nucleases Cas9 and Cas12a. Biochem Soc Trans. 2020 Feb. 28; 48(1):207-219. doi: 10.1042/BST20190563. PMID: 31872209; PMCID: PMC7054755; (4) Gaillochet C. Peña Fernández A, Goossens V, D'Halluin K, Drozdzecki A, Shafie M, Van Duyse J, Van Isterdael G, Gonzalez C, Vermeersch M, De Saeger J, Develtere W, Audenaert D, De Vleesschauwer D, Meulewaeter F, Jacobs T B. Systematic optimization of Cas12a base editors in wheat and maize using the ITER platform. Genome Biol. 2023 Jan. 13; 24(1):6. doi: 10.1186/s13059-022-02836-2. PMID: 36639800; PMCID: PMC9838060; each of which are incorporated herein by reference in their entireties.

Base editing was first described in Komor et al., “Programmable editing of a target base in genomic DNA without double-stranded DNA cleavage,” Nature, May 19, 2016, 533(7603); pp. 420-424 in the form of cytosine base editors or CBEs followed by the disclosure of Gaudelli et al., “Programmable base editing of A-T to G-C in genomic DNA without DNA cleavage,” Nature, Vol. 551, pp. 464-471 describing adenine base editors or ABEs. Subsequently, base editing has been described in numerous scientific publications, including, but not limited to (i) Kim J S. Precision genome engineering through adenine and cytosine base editing. Nat Plants. 2018 March; 4(3):148-151. doi: 10.1038/s41477-018-0115-z. Epub 2018 Feb. 26. PMID: 29483683.; (ii) Wei Y, Zhang X H, Li D L. The “new favorite” of gene editing technology-single base editors. Yi Chuan. 2017 Dec. 20; 39(12):1115-1121. doi: 10.16288/j.yczz.17-389. PMID: 29258982; (iii) Tang J, Lee T, Sun T. Single-nucleotide editing: From principle, optimization to application. Hum Mutat. 2019 December; 40(12):2171-2183. doi: 10.1002/humu.23819. Epub 2019 Sep. 15. PMID: 31131955; PMCID: PMC6874907; (iv) Grünewald J, Zhou R, Lareau C A, Garcia S P, Iyer S, Miller B R, Langner L M, Hsu J Y, Aryce M J, Joung J K. A dual-deaminase CRISPR base editor enables concurrent adenine and cytosine editing. Nat Biotechnol. 2020 July; 38(7):861-864. doi: 10.1038/s41587-020-0535-y. Epub 2020 Jun. 1. PMID: 32483364; PMCID: PMC7723518; (v) Sakata R C, Ishiguro S, Mori H, Tanaka M, Tatsuno K, Ueda H, Yamamoto S, Seki M, Masuyama N, Nishida K, Nishimasu H, Arakawa K, Kondo A, Nureki O, Tomita M, Aburatani H, Yachie N. Base editors for simultaneous introduction of C-to-T and A-to-G mutations. Nat Biotechnol. 2020 July; 38(7):865-869. doi: 10.1038/s41587-020-0509-0. Epub 2020 Jun. 2. Erratum in: Nat Biotechnol. 2020 Jun. 5; PMID: 32483365; (vi) Fan J, Ding Y, Ren C, Song Z, Yuan J, Chen Q, Du C, Li C, Wang X, Shu W. Cytosine and adenine deaminase base-editors induce broad and nonspecific changes in gene expression and splicing. Commun Biol. 2021 Jul. 16; 4(1):882. doi: 10.1038/s42003-021-02406-5. PMID: 34272468; PMCID: PMC8285404; (vii) Zhang S, Yuan B, Cao J, Song L, Chen J, Qiu J, Qiu Z, Zhao X M, Chen J, Cheng T L. TadA orthologs enable both cytosine and adenine editing of base editors. Nat Commun. 2023 Jan. 26; 14(1):414. doi: 10.1038/s41467-023-36003-3. PMID: 36702837; PMCID: PMC988000; and (viii) Zhang S, Song L, Yuan B, Zhang C, Cao J, Chen J, Qiu J, Tai Y, Chen J, Qiu Z, Zhao X M, Cheng T L. TadA reprogramming to generate potent miniature base editors with high precision. Nat Commun. 2023 Jan. 26; 14(1):413. doi: 10.1038/s41467-023-36004-2. PMID: 36702845; PMCID: PMC987999, each of which are incorporated herein by reference in their entireties.

Amino acid and nucleotide sequences of base editors, including adenosine base editors, cytidine base editors, and others are readily available in the art. For example, exemplary base editors that may be delivered using the LNP compositions described herein can be found in the following published patent applications, each of their contents (including any and all biological sequences) are incorporated herein by reference:

| | |
|---|---|
| US 2023/0021641 A1 | CAS9 VARIANTS HAVING NON-CANONICAL |
| | PAM SPECIFICITIES AND USES THEREOF |
| U.S. Pat. No. 11,542,496 B2 | Cytosine to guanine base editor |
| U.S. Pat. No. 11,542,509 B2 | Incorporation of unnatural amino acids |
| | into proteins using base editing |
| US 2022/0315906 A1 | BASE EDITORS WITH DIVERSIFIED TARGETING SCOPE |
| US 2022/0282275 A1 | G-TO-T BASE EDITORS AND USES THEREOF |
| US 2022/0249697 A1 | AAV DELIVERY OF NUCLEOBASE EDITORS |

In some embodiments, the LNP cargo comprises a base editing system or a polynucleotide encoding a CRISPR-Cas base editing system. In some embodiments, the cargo comprises a component of a base editing system or a polynucleotide encoding a component of a base editing system.

Base editing does not require double-stranded DNA breaks or a DNA donor template. In some embodiments, base editing comprises creating an SSB in a target double-stranded DNA sequence and then converting a nucleobase. In some embodiments, the nucleobase conversion is an adenosine to a guanine. In some embodiments, the nucleobase conversion is a thymine to a cytosine. In some embodiments, the nucleobase conversion is a cytosine to a thymine. In some embodiments, the nucleobase conversion is a guanine to an adenosine. In some embodiments, the nucleobase conversion is an adenosine to inosine. In some embodiments, the nucleobase conversion is a cytosine to uracil.

A base editing system comprises a base editor which can convert a nucleobase. The base editor (“BE”) comprises a partially inactive Cas protein which is connected to a deaminase that precisely and permanently edits a target nucleobase in a polynucleotide sequence. A base editor comprises a polynucleotide programmable nucleotide binding domain and a nucleobase editing domain (e.g., adenosine deaminase or cytosine deaminase). In some embodiments, the partially inactive Cas protein is a Cas nickase. In some embodiments, the partially inactive Cas protein is a Cas9 nickase (also referred to as “nCas9”).

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleobase and bases of the target polynucleotide sequence) and thereby localize the nucleobase editor to the target polynucleotide sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In certain embodiments, polynucleotide programmable nucleotide binding domains also include nucleobase programmable proteins that bind RNA. In certain embodiments, the polynucleotide programmable nucleotide binding domain can be associated with a nucleobase that guides the polynucleotide programmable nucleotide binding domain to an RNA.

In some embodiments, the LNP-deliverable base editors may comprise a deaminase domain that is a cytidine deaminase domain. A cytidine deaminase domain may also be referred to interchangeably as a cytosine deaminase domain. In some embodiments, the cytidine deaminase catalyzes the hydrolytic deamination of cytidine (C) or deoxycytidine (dC) to uridine (U) or deoxyuridine (dU), respectively. In some embodiments, the cytidine deaminase domain catalyzes the hydrolytic deamination of cytosine (C) to uracil (U). In some embodiments, the cytidine deaminase catalyzes the hydrolytic deamination of cytidine or cytosine in deoxyribonucleic acid (DNA). Without wishing to be bound by any particular theory, fusion proteins comprising a cytidine deaminase are useful inter alia for targeted editing, referred to herein as “base editing,” of nucleic acid sequences in vitro and in vivo.

One exemplary suitable type of cytidine deaminase is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner (see, e.g., Conticello S G. The AID/APOBEC family of nucleic acid mutators. Genome Biol. 2008; 9(6):229). One family member, activation-induced cytidine deaminase (AID), is responsible for the maturation of antibodies by converting cytosines in ssDNA to uracils in a transcription-dependent, strand-biased fashion (see, e.g., Reynaud C A, et al. What role for AID: mutator, or assembler of the immunoglobulin mutasome, Nat Immunol. 2003; 4(7):631-638). The apolipoprotein B editing complex 3 (APOBEC3) enzyme provides protection to human cells against a certain HIV-1 strain via the deamination of cytosines in reverse-transcribed viral ssDNA (see, e.g., Bhagwat A S. DNA-cytosine deaminases: from antibody maturation to antiviral defense. DNA Repair (Amst). 2004; 3(1):85-89).

Some aspects of this disclosure relate to the recognition that the activity of cytidine deaminase enzymes such as APOBEC enzymes can be directed to a specific site in genomic DNA. Without wishing to be bound by any particular theory, advantages of using a nucleic acid programmable binding protein (e.g., a Cas9 domain) as a recognition agent include (1) the sequence specificity of nucleic acid programmable binding protein (e.g., a Cas9 domain) can be easily altered by simply changing the sgRNA sequence; and (2) the nucleic acid programmable binding protein (e.g., a Cas9 domain) may bind to its target sequence by denaturing the dsDNA, resulting in a stretch of DNA that is single-stranded and therefore a viable substrate for the deaminase. It should be understood that other catalytic domains of napDNAbps, or catalytic domains from other nucleic acid editing proteins, can also be used to generate fusion proteins with Cas9, and that the disclosure is not limited in this regard.

In some embodiments, the cytidine deaminase is an apolipoprotein B mRNA-editing complex (APOBEC) family deaminase. In some embodiments, the cytidine deaminase is an APOBEC1 deaminase. In some embodiments, the cytidine deaminase is an APOBEC2 deaminase. In some embodiments, the cytidine deaminase is an APOBEC3 deaminase. In some embodiments, the cytidine deaminase is an APOBEC3A deaminase. In some embodiments, the cytidine deaminase is an APOBEC3B deaminase. In some embodiments, the cytidine deaminase is an APOBEC3C deaminase. In some embodiments, the cytidine deaminase is an APOBEC3D deaminase. In some embodiments, the cytidine deaminase is an APOBEC3E deaminase. In some embodiments, the cytidine deaminase is an APOBEC3F deaminase. In some embodiments, the cytidine deaminase is an APOBEC3G deaminase. In some embodiments, the cytidine deaminase is an APOBEC3H deaminase. In some embodiments, the cytidine deaminase is an APOBEC4 deaminase. In some embodiments, the cytidine deaminase is an activation-induced deaminase (AID). In some embodiments, the cytidine deaminase is a vertebrate cytidine deaminase. In some embodiments, the cytidine deaminase is an invertebrate cytidine deaminase. In some embodiments, the cytidine deaminase is a human, chimpanzee, gorilla, monkey, cow, dog, rat, or mouse deaminase. In some embodiments, the cytidine deaminase is a human cytidine deaminase. In some embodiments, the cytidine deaminase is a rat cytidine deaminase, e.g., rAPOBEC1.

In some embodiments, the nucleic acid editing domain is at least 80%, at least 85%, at least 90%, at least 92%, at least 95%, at least 96%, at least 97%, at least 98%, at least 99%, or at least 99.5% identical to any of the cytidine deaminase domain examples above.

In other embodiments, the LNP-deliverable base editors may comprise a deaminase domain that is an adenosine deaminase domain.

The disclosure provides fusion proteins that comprise one or more adenosine deaminases. In some aspects, such fusion proteins are capable of deaminating adenosine in a nucleic acid sequence (e.g., DNA or RNA). As one example, any of the fusion proteins provided herein may be base editors, (e.g., adenine base editors). Without wishing to be bound by any particular theory, dimerization of adenosine deaminases (e.g., in cis or in trans) may improve the ability (e.g., efficiency) of the fusion protein to modify a nucleic acid base, for example to deaminate adenine. In some embodiments, any of the fusion proteins may comprise 2, 3, 4 or 5 adenosine deaminases. In some embodiments, any of the fusion proteins provided herein comprise two adenosine deaminases. Exemplary, non-limiting, embodiments of adenosine deaminases are provided herein. It should be appreciated that the mutations provided herein (e.g., mutations in ecTadA) may be applied to adenosine deaminases in other adenosine base editors, for example those provided in U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, all of which are incorporated herein by reference in their entireties.

In some embodiments, any of the adenosine deaminases provided herein is capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aurcus, Salmonella typhi, Shewanella putrefaciens, Haemophilus influenzae, Caulobacter crescentus, or Bacillus subtilis. In some embodiments, the adenosine deaminase is from E. coli.

Any two or more of the adenosine deaminases described herein may be connected to one another (e.g. by a linker) within an adenosine deaminase domain of the fusion proteins provided herein. For instance, the fusion proteins provided herein may contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase. In some embodiments, the fusion protein comprises two adenosine deaminases (e.g., a first adenosine deaminase and a second adenosine deaminase). In some embodiments, the fusion protein comprises a first adenosine deaminase and a second adenosine deaminase. In some embodiments, the first adenosine deaminase is N-terminal to the second adenosine deaminase in the fusion protein. In some embodiments, the first adenosine deaminase is C-terminal to the second adenosine deaminase in the fusion protein. In some embodiments, the first adenosine deaminase and the second deaminase are fused directly or via a linker.

In some embodiments, the base editor comprises a deaminase enzyme. In some embodiments, the base editor comprises a cytidine deaminase. In some embodiments, the base editor comprises a Cas9 protein fused to a cytidine deaminase enzyme. In some embodiments, the base editor comprises an adenosine deaminase. In some embodiments, the base editor comprises a Cas9 protein fused to an adenosine deaminase enzyme.

In some embodiments, the base editing system comprises an uracil glycosylase inhibitor. In some embodiments, the base editing system comprises a Cas9 protein fused to an uracil glycosylase inhibitor. In some embodiments, the cargo comprises an uracil glycosylase inhibitor or a polynucleotide encoding an uracil glycosylase inhibitor. In some embodiments, the cargo comprises a Cas9 protein fused to an uracil glycosylase inhibitor or a polynucleotide encoding a Cas9 protein fused to an uracil glycosylase inhibitor.

A variety of nucleobase modifying enzymes are suitable for use in the nucleobase systems disclosed herein. In some embodiments, the nucleobase modifying enzyme is a RNA base editor. In some embodiments, the RNA base editor can be a cytidine deaminase, which converts cytidine into uridine. Non-limiting examples of cytidine deaminases include cytidine deaminase 1 (CDA1), cytidine deaminase 2 (CDA2), activation-induced cytidine deaminase (AICDA), apolipoprotein B mRNA-editing complex (APOBEC) family cytidine deaminase (e.g., APOBEC1, APOBEC2, APOBEC3A, APOBEC3B, APOBEC3C, APOBEC3D/E, APOBEC3F, APOBEC3G, APOBEC3H, APOBEC4), APOBEC1 complementation factor/APOBEC1 stimulating factor (ACF1/ASF) cytidine deaminase, cytosine deaminase acting on RNA (CDAR), bacterial long isoform cytidine deaminase (CDDL), and cytosine deaminase acting on tRNA (CDAT). In other embodiments, the RNA base editor can be an adenosine deaminase, which converts adenosine into inosine, which is read by polymerase enzymes as guanosine. In certain embodiments, adenosine deaminases include tRNA adenine deaminase, adenosine deaminase, adenosine deaminase acting on RNA (ADAR), and adenosine deaminase acting on tRNA (ADAT).

In some embodiments, in the nucleobase editing systems disclosed herein, the Cas effector may associate with one or more functional domains (e.g., via fusion protein or suitable linkers). In some embodiments, the effector domain comprises one or more cytidine or nucleotide deaminases that mediate editing of via hydrolytic deamination. In certain embodiments, the effector domain comprises the adenosine deaminase acting on RNA (ADAR) family of enzymes. In certain embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu) ADAR1 or (hu) ADAR2, preferably huADAR2 or catalytic domain thereof.

In some embodiments, the cytidine deaminase is a human, rat or lamprey cytidine deaminase. In some embodiments, the cytidine deaminase is an apolipoprotein B mRNA-editing complex (APOBEC) family deaminase, an activation-induced deaminase (AID), or a cytidine deaminase 1 (CDA1).

In certain embodiments, the adenosine deaminase is adenosine deaminase acting on RNA (ADAR). In certain embodiments, the ADAR is ADAR (ADAR1), ADARB1 (ADAR2) or ADARB2 (ADAR3) (see, e.g., Savva et al. Genon. Biol. 2012, 13(12):252).

In some embodiments, the gene editing system comprises AID/APOBEC (apolipoprotein B editing complex) family of enzymes deaminates cytidine to uridine, leading to mutations in RNA and DNA.

In some embodiments, the nucleobase editing system comprises ADAR and an antisense oligonucleotide. In certain embodiments, the antisense oligonucleotide is chemically optimized antisense oligonucleotide. In certain embodiments, the antisense oligonucleotide is administered for the nucleobase editing, wherein the antisense oligonucleotide activates human endogenous ADAR for nucleobase editing. Such ADAR and antisense oligonucleotide editing system provides a safer site-directed RNA editing with low off-target effect. See, e.g., Merkle et al., Nature Biotechnology, 2019, 37, 133-138.

Any of the above base editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

### Prime Editors

In various embodiments, the herein disclosed LNPs may contain a prime editing system or components thereof and which may be used to conduct prime editing of target nucleic acid sequences in cells, tissues, and organs in an ex vivo or in vivo manner.

Prime editing technology is a gene editing technology that can make targeted insertions, deletions, and all transversion and transition point mutations in a target genome. Without wishing to be bound by any particular theory, the prime editing process may search and replace endogenous sequences in a target polynucleotide. The spacer sequence of a prime editing guide RNA (“PERNA” or “pegRNA”) recognizes and anneals with a search target sequence in a target strand of a double stranded target polynucleotide, e.g., a double stranded target DNA. A prime editing complex may generate a nick in the target DNA on the edit strand which is the complementary strand of the target strand. The prime editing complex may then use a free 3′ end formed at the nick site of the edit strand to initiate DNA synthesis, where a “primer binding site sequence” (PBS) of the PEgRNA complexes with the free 3′ end, and a single stranded DNA is synthesized (by reverse transcriptase) using an editing template of the PERNA as a template. As used herein, a “primer binding site” is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site.

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

The editing template may comprise one or more intended nucleotide edits compared to the endogenous double stranded target DNA sequence. Accordingly, the newly synthesized single stranded DNA also comprises the nucleotide edit(s) encoded by the editing template. Through removal of the editing target sequence on the edit strand of the double stranded target DNA and DNA repair mechanism, the newly synthesized single stranded DNA replaces the editing target sequence, and the desired nucleotide edit(s) are incorporated into the double stranded target DNA.

Prime editing was first described in Anzalone et al., “Search-and-replace genome editing without double-strand breaks or donor DNA,” Nature, December 2019, 576(7789): pp. 149-157, which is incorporated herein in its entirety. Prime editing has subsequently been described and detailed in numerous follow-on publications, including, for example, (i) Liu et al., “Prime editing: a search and replace tool with versatile base changes,” Yi Chuan, Nov. 20, 2022, 44(11):993-1008; (ii) Lu C et al., “Prime Editing: An All-Rounder for Genome Editing. Int J Mol Sci. 2022 Aug. 30; 23(17):9862; (iii) Velimirovic M, Zanetti L C, Shen M W, Fife J D, Lin L, Cha M, Akinci E, Barnum D, Yu T, Sherwood R I. Peptide fusion improves prime editing efficiency. Nat Commun. 2022 Jun. 18; 13(1):3512. doi: 10.1038/s41467-022-31270-y. PMID: 35717416; PMCID: PMC9206660; (iv) Velimirovic M, Zanetti L C, Shen M W, Fife J D, Lin L, Cha M, Akinci E, Barnum D, Yu T, Sherwood R I. Peptide fusion improves prime editing efficiency. Nat Commun. 2022 Jun. 18; 13(1):3512. doi: 10.1038/s41467-022-31270-y. PMID: 35717416; PMCID: PMC9206660; (v) Habib O, Habib G, Hwang G H, Bae S. Comprehensive analysis of prime editing outcomes in human embryonic stem cells. Nucleic Acids Res. 2022 Jan. 25; 50(2):1187-1197. doi: 10.1093/nar/gkab1295. PMID: 35018468; PMCID: PMC8789035; (vi) Marzec M, Brąszewska-Zalewska A, Hensel G. Prime Editing: A New Way for Genome Editing. Trends Cell Biol. 2020 April; 30(4):257-259. doi: 10.1016/j.tcb.2020.01.004. Epub 2020 Jan. 27. PMID: 32001098; (vii) Tao R, Wang Y, Jiao Y, Hu Y, Li L, Jiang L, Zhou L, Qu J, Chen Q, Yao S. Bi-PE: bi-directional priming improves CRISPR/Cas9 prime editing in mammalian cells. Nucleic Acids Res. 2022 Jun. 24; 50(11):6423-6434. doi: 10.1093/nar/gkac506. PMID: 35687127; PMCID: PMC9226529; (viii) Nelson J W, Randolph P B, Shen S P, Everette K A, Chen P J, Anzalone A V, An M, Newby G A, Chen J C, Hsu A, Liu D R. Engineered pegRNAs improve prime editing efficiency. Nat Biotechnol. 2022 March; 40(3):402-410. doi: 10.1038/s41587-021-01039-7. Epub 2021 Oct. 4. Erratum in: Nat Biotechnol. 2021 Dec. 8; PMID: 34608327; PMCID: PMC8930418; (ix) Doman J L, Sousa A A, Randolph P B, Chen P J, Liu D R. Designing and executing prime editing experiments in mammalian cells. Nat Protoc. 2022 November; 17(11):2431-2468. doi: 10.1038/s41596-022-00724-4. Epub 2022 Aug. 8. PMID: 35941224; PMCID: PMC9799714; (x) Jiao Y, Zhou L, Tao R, Wang Y, Hu Y, Jiang L, Li L, Yao S. Random-PE: an efficient integration of random sequences into mammalian genome by prime editing. Mol Biomed. 2021 Nov. 18; 2(1):36. doi: 10.1186/s43556-021-00057-w. PMID: 35006470; PMCID: PMC8607425; and (xi) Awan M J A, Ali Z, Amin I, Mansoor S. Twin prime editor: seamless repair without damage. Trends Biotechnol. 2022 April; 40(4):374-376. doi: 10.1016/j.tibtech.2022.01.013. Epub 2022 Feb. 10. PMID: 35153078, all of which are incorporated herein by reference.

In addition, prime editing has been described and disclosed in numerous published patent applications, each of which their entire contents, amino acid sequences, nucleotide sequences, and all disclosures therein are incorporated herein by reference in their entireties:

| Publication No. | Publication Date | Title |
|---|---|---|
| WO 2023/015309 A2 | Feb. 9, 2023 | IMPROVED PRIME EDITORS AND METHODS |
| | | OF USE |
| WO 2023/004439 A2 | Jan. 26, 2023 | GENOME EDITING COMPOSITIONS AND |
| | | METHODS FOR TREATMENT OF CHRONIC |
| | | GRANULOMATOUS DISEASE |
| WO 2023/288332 A2 | Jan. 19, 2023 | GENOME EDITING COMPOSITIONS AND |
| | | METHODS FOR TREATMENT OF WILSON'S |
| | | DISEASE |
| WO 2023/283092 A1 | Jan. 12, 2023 | COMPOSITIONS AND METHODS FOR |
| | | EFFICIENT GENOME EDITING |
| WO 2023/283246 A1 | Jan. 12, 2023 | MODULAR PRIME EDITOR SYSTEMS FOR |
| | | GENOME ENGINEERING |
| WO 2022/256714 A3 | Jan. 12, 2023 | GENOME EDITING COMPOSITIONS AND |
| | | METHODS FOR TREATMENT OF WILSON'S |
| | | DISEASE |
| EP 4107273 A1 | Dec. 28, 2022 | PRIME EDITING TECHNOLOGY FOR PLANT |
| | | GENOME ENGINEERING |
| WO 2022/256714 A2 | Dec. 8, 2022 | GENOME EDITING COMPOSITIONS AND |
| | | METHODS FOR TREATMENT OF WILSON'S |
| | | DISEASE |
| WO 2022/234051 A1 | Nov. 10, 2022 | SPLIT PRIME EDITING ENZYME |
| US 2022/0356469 A1 | Nov. 10, 2022 | METHODS AND COMPOSITIONS FOR EDITING |
| | | NUCLEOTIDE SEQUENCES METHODS AND |
| | | COMPOSITIONS FOR EDITING NUCLEOTIDE |
| | | SEQUENCES |
| WO 2022/206352 A1 | Oct. 6, 2022 | PRIME EDITING TOOL, FUSION RNA, AND |
| | | USE THEREOF |
| WO 2022/212926 A1 | Oct. 6, 2022 | METHODS AND COMPOSITIONS FOR EDITING |
| | | NUCLEOTIDE SEQUENCES |
| WO 2022/204476 A1 | Sep. 29, 2022 | NUCLEOTIDE EDITING TO REFRAME DMD |
| | | TRANSCRIPTS BY BASE EDITING AND PRIME |
| | | EDITING |
| WO 2022/203905 A1 | Sep. 29, 2022 | PRIME EDITING-BASED SIMULTANEOUS |
| | | GENOMIC DELETION AND INSERTION |
| U.S. Pat. No. 11,447,770 B1 | Sep. 20, 2022 | Methods and compositions for prime editing |
| | | nucleotide sequences |
| WO 2022/174829 A1 | Aug. 25, 2022 | EDITING OF DOUBLE-STRANDED DNA WITH |
| | | RELAXED PAM REQUIREMENT FIELD OF THE |
| | | DISCLOSURE |
| WO 2022/170058 A1 | Aug. 11, 2022 | PRIME EDITOR SYSTEM FOR IN VIVO |
| | | GENOME EDITING |
| WO 2022/169235 A1 | Aug. 11, 2022 | PRIME EDITING COMPOSITION WITH |
| | | IMPROVED EDITING EFFICIENCY |
| WO 2022/150790 A3 | Aug. 11, 2022 | PRIME EDITOR VARIANTS, CONSTRUCTS, |
| | | AND METHODS FOR ENHANCING PRIME |
| | | EDITING EFFICIENCY AND PRECISION |
| WO 2022/149166 A1 | Jul. 14, 2022 | A COCKTAIL FORMULATION FOR SELECTIVE |
| | | ENRICHMENT OF GENE-MODIFIED CELLS |
| WO 2022/150790 A2 | Jul. 14, 2022 | PRIME EDITOR VARIANTS, CONSTRUCTS, |
| | | AND METHODS FOR ENHANCING PRIME |
| | | EDITING EFFICIENCY AND PRECISION |
| U.S. Pat. No. 11,384,353 B2 | Jul. 12, 2022 | Inhibition of unintended mutations in gene editing |
| WO 2022/067130 A3 | Jun. 23, 2022 | PRIME EDITING GUIDE RNAS, |
| | | COMPOSITIONS THEREOF, AND METHODS OF |
| | | USING THE SAME |
| WO 2022/114815 A1 | Jun. 2, 2022 | COMPOSITION FOR PRIME EDITING |
| | | COMPRISING TRANS-SPLICING ADENO- |
| | | ASSOCIATED VIRUS VECTOR |
| WO 2022/100662 A1 | May 19, 2022 | GENOMIC EDITING OF IMPROVED |
| | | EFFICIENCY AND ACCURACY |
| WO 2022/098765 A1 | May 12, 2022 | SPLIT PRIME EDITING PLATFORMS |
| WO 2022/098885 A1 | May 12, 2022 | PRECISE GENOME DELETION AND |
| | | REPLACEMENT METHOD BASED ON PRIME |
| | | EDITING |
| WO 2022/071745 A1 | Apr. 7, 2022 | PRIME EDITING USING HIV REVERSE |
| | | TRANSCRIPTASE AND CAS9 OR VARIANT |
| | | THEREOF |
| WO 2022/067130 A2 | Mar. 31, 2022 | PRIME EDITING GUIDE RNAS, |
| | | COMPOSITIONS THEREOF, AND METHODS OF |
| | | USING THE SAME |
| WO 2022/065689 A1 | Mar. 31, 2022 | PRIME EDITING-BASED GENE EDITING |
| | | COMPOSITION WITH ENHANCED EDITING |
| | | EFFICIENCY AND USE THEREOF |
| US 2022/0064626 A1 | Mar. 3, 2022 | INHIBITION OF UNINTENDED MUTATIONS IN |
| | | GENE EDITING |
| WO 2022/032085 A1 | Feb. 10, 2022 | TARGETED SEQUENCE INSERTION |
| | | COMPOSITIONS AND METHODS |
| WO 2022/025623 A1 | Feb. 3, 2022 | SYSTEM AND METHOD FOR PRIME EDITING |
| | | EFFICIENCY PREDICTION USING DEEP |
| | | LEARNING |
| WO 2021/226558 A8 | Jan. 13, 2022 | METHODS AND COMPOSITIONS FOR |
| | | SIMULTANEOUS EDITING OF BOTH STRANDS |
| | | OF A TARGET DOUBLE-STRANDED |
| | | NUCLEOTIDE SEQUENCE |
| WO 2021/243289 A1 | Dec. 2, 2021 | SYSTEMS AND METHODS FOR STABLE AND |
| | | HERITABLE ALTERATION BY PRECISION |
| | | EDITING (SHAPE) |
| WO 2021/226558 A1 | Nov. 11, 2021 | METHODS AND COMPOSITIONS FOR |
| | | SIMULTANEOUS EDITING OF BOTH STRANDS |
| | | OF A TARGET DOUBLE-STRANDED |
| | | NUCLEOTIDE SEQUENCE |
| WO 2021/215897 A1 | Oct. 28, 2021 | GENOME EDITION USING CAS9 OR CAS9 |
| | | VARIANT |
| WO 2021/215827 A1 | Oct. 28, 2021 | GENOME EDITING USING CAS9 OR CAS9 |
| | | VARIANT |
| WO 2020/191248 A8 | Oct. 21, 2021 | METHODS AND COMPOSITIONS FOR EDITING |
| | | NUCLEOTIDE SEQUENCES |
| WO 2020/191234 A8 | Oct. 21, 2021 | METHODS AND COMPOSITIONS FOR EDITING |
| | | NUCLEOTIDE SEQUENCES |
| WO 2021/165508 A1 | Aug. 26, 2021 | PRIME EDITING TECHNOLOGY FOR PLANT |
| | | GENOME ENGINEERING |
| WO 2021/138469 A1 | Jul. 8, 2021 | GENOME EDITING USING REVERSE |
| | | TRANSCRIPTASE ENABLED AND FULLY |
| | | ACTIVE CRISPR COMPLEXES |
| WO 2021/092204 A1 | May 14, 2021 | METHODS AND COMPOSITIONS FOR |
| | | NUCLEIC ACID-GUIDED NUCLEASE CELL |
| | | TARGETING SCREEN |
| WO 2021/076876 A1 | Apr. 22, 2021 | GENOTYPING EDITED MICROBIAL STRAINS |
| WO 2021/072328 A1 | Apr. 15, 2021 | METHODS AND COMPOSITIONS FOR PRIME |
| | | EDITING RNA |
| WO 2020/191153 A8 | Dec. 30, 2020 | METHODS AND COMPOSITIONS FOR EDITING |
| | | NUCLEOTIDE SEQUENCES |
| WO 2020/191153 A3 | Dec. 10, 2020 | METHODS AND COMPOSITIONS FOR EDITING |
| | | NUCLEOTIDE SEQUENCES |
| WO 2020/191153 A9 | Nov. 12, 2020 | METHODS AND COMPOSITIONS FOR EDITING |
| | | NUCLEOTIDE SEQUENCES |
| WO 2020/191171 A9 | Oct. 29, 2020 | METHODS AND COMPOSITIONS FOR EDITING |
| | | NUCLEOTIDE SEQUENCES |
| WO 2020/191248 A1 | Sep. 24, 2020 | METHOD AND COMPOSITIONS FOR EDITING |
| | | NUCLEOTIDE SEQUENCES |
| WO 2020/191239 A1 | Sep. 24, 2020 | METHODS AND COMPOSITIONS FOR EDITING |
| | | NUCLEOTIDE SEQUENCES |
| WO 2020/191153 A2 | Sep. 24, 2020 | METHODS AND COMPOSITIONS FOR EDITING |
| | | NUCLEOTIDE SEQUENCES |
| WO 2020/191246 A1 | Sep. 24, 2020 | METHODS AND COMPOSITIONS FOR EDITING |
| | | NUCLEOTIDE SEQUENCES |
| WO 2020/191249 A1 | Sep. 24, 2020 | METHODS AND COMPOSITIONS FOR EDITING |
| | | NUCLEOTIDE SEQUENCES |
| WO 2020/191233 A1 | Sep. 24, 2020 | METHODS AND COMPOSITIONS FOR EDITING |
| | | NUCLEOTIDE SEQUENCES |
| WO 2020/191243 A1 | Sep. 24, 2020 | METHODS AND COMPOSITIONS FOR EDITING |
| | | NUCLEOTIDE SEQUENCES |
| WO 2020/191234 A1 | Sep. 24, 2020 | METHODS AND COMPOSITIONS FOR EDITING |
| | | NUCLEOTIDE SEQUENCES |
| WO 2020/191245 A1 | Sep. 24, 2020 | METHODS AND COMPOSITIONS FOR EDITING |
| | | NUCLEOTIDE SEQUENCES |
| WO 2020/191242 A1 | Sep. 24, 2020 | METHODS AND COMPOSITIONS FOR EDITING |
| | | NUCLEOTIDE SEQUENCES |
| WO 2020/191171 A1 | Sep. 24, 2020 | METHODS AND COMPOSITIONS FOR EDITING |
| | | NUCLEOTIDE SEQUENCES |
| WO 2020/191241 A1 | Sep. 24, 2020 | METHODS AND COMPOSITIONS FOR EDITING |
| | | NUCLEOTIDE SEQUENCES |
| WO 2020/156575 A1 | Aug. 6, 2020 | INHIBITION OF UNINTENDED MUTATIONS IN |
| | | GENE EDITING |
| U.S. Pat. No. 10,189,831 B2 | Jan. 29, 2019 | Non-nucleoside reverse transcriptase inhibitors |
| WO 2019/014564 A1 | Jan. 17, 2019 | SYSTEMS AND METHODS FOR TARGETED |
| | | INTEGRATION AND GENOME EDITING AND |
| | | DETECTION THEREOF USING INTEGRATED |
| | | PRIMING SITES |
| U.S. Pat. No. 10,150,955 B2 | Dec. 11, 2018 | Stabilized reverse transcriptase fusion proteins |
| WO 2018/049168 A1 | Mar. 15, 2018 | HIGH-THROUGHPUT PRECISION GENOME |
| | | EDITING |
| U.S. Pat. No. 9,783,791 B2 | Oct. 10, 2017 | Mutant reverse transcriptase and methods of use |
| U.S. Pat. No. 9,458,484 B2 | Oct. 4, 2016 | Reverse transcriptase mixtures with improved |
| | | storage stability |
| WO 2023/070110 A2 | Apr. 27, 2023 | GENOME EDITING COMPOSITIONS AND |
| | | METHODS FOR TREATMENT OF RETINITIS |
| | | PIGMENTOSA |
| US 2023/0059368 A1 | Feb. 23, 2023 | POLYNUCLEOTIDE EDITORS AND METHODS |
| | | OF USING THE SAME |
| WO 2023/086558 A1 | May 19, 2023 | GENOME EDITING COMPOSITIONS AND |
| | | METHODS FOR TREATMENT OF FRAGILE X |
| | | SYNDROME |
| WO 2023/288332 A3 | Feb. 23, 2023 | GENOME EDITING COMPOSITIONS AND |
| | | METHODS FOR TREATMENT OF WILSON'S |
| | | DISEASE |
| WO 2023/192655 A2 | Oct. 5, 2023 | METHODS AND COMPOSITIONS FOR EDITING |
| | | NUCLEOTIDE SEQUENCES |
| WO 2022/256714 A2 | Dec. 8, 2022 | GENOME EDITING COMPOSITIONS AND |
| | | METHODS FOR TREATMENT OF WILSON'S |
| | | DISEASE |
| WO 2023/283092 A1 | Jan. 12, 2023 | COMPOSITIONS AND METHODS FOR |
| | | EFFICIENT GENOME EDITING |
| WO 2023/070062 A3 | May 25, 2023 | GENOME EDITING COMPOSITIONS AND |
| | | METHODS FOR TREATMENT OF USHER |
| | | SYNDROME TYPE 3 |
| WO 2023/081787 A3 | Sep. 28, 2023 | GENOME EDITING COMPOSITIONS AND |
| | | METHODS FOR TREATMENT OF FANCONI |
| | | ANEMIA |
| WO 2023/086389 A1 | May 19, 2023 | GENOME EDITING COMPOSITIONS AND |
| | | METHODS FOR TREATMENT OF |
| | | AMYOTROPHIC LATERAL SCLEROSIS |
| WO 2023/096847 A2 | Jun. 1, 2023 | METHODS AND COMPOSITIONS FOR |
| | | INHIBITING MISMATCH REPAIR |
| WO 2023/081426 A1 | May 11, 2023 | GENOME EDITING COMPOSITIONS AND |
| | | METHODS FOR TREATMENT OF |
| | | FRIEDREICH'S ATAXIA |
| WO 2023/015318 A2 | Feb. 9, 2023 | GENOME EDITING COMPOSITIONS AND |
| | | METHODS FOR TREATMENT OF CYSTIC |
| | | FIBROSIS |
| WO 2022/212926 A1 | Oct. 6, 2022 | METHODS AND COMPOSITIONS FOR EDITING |
| | | NUCLEOTIDE SEQUENCES |
| WO 2023/028180 A3 | Aug. 24, 2023 | GENOME EDITING COMPOSITIONS AND |
| | | METHODS FOR TREATMENT OF |
| | | RETINOPATHY |
| WO 2023/015014 A1 | Feb. 9, 2023 | GENOME EDITING COMPOSITIONS AND |
| | | METHODS FOR TREATMENT OF MYOTONIC |
| | | DYSTROPHY |
| WO 2023/086842 A1 | May 19, 2023 | GENOME EDITING COMPOSITIONS AND |
| | | METHODS FOR TREATMENT OF FUCHS |
| | | ENDOTHELIAL CORNEAL DYSTROPHY |
| WO 2023/096992 A1 | Jun. 1, 2023 | GENOME EDITING COMPOSITIONS AND |
| | | METHODS FOR TREATMENT OF GLYCOGEN |
| | | STORAGE DISEASE TYPE 1B |
| WO 2023/096977 A2 | Jun. 1, 2023 | MODIFIED PRIME EDITING GUIDE RNAS |

In some embodiments, the cargo comprises a prime editing system or a polynucleotide encoding a prime editing system. In some embodiments, the cargo comprises a component of a prime editing system or a polynucleotide encoding a component of a prime editing system.

Prime editing is a versatile and precise genome editing method that directly writes new genetic information into a specified DNA site using a catalytically impaired Cas fused to an engineered reverse transcriptase, also referred to as a prime editor, which is programmable using a prime editing guide RNA (“pegRNA”) that both specifies the target site and encodes the desired edit (see, e.g., Anzalone et al., Nature 2019). Prime editing bypasses the need for DNA donor templates by using a prime editor having nickase or catalytically impaired enzymatic activity.

A prime editing system comprises a prime editor. The prime editor (“PE”) comprises a catalytically impaired Cas protein fused to an engineered reverse transcriptase which can precisely and permanently edit one or more target nucleobases in a target polynucleotide.

In some embodiments, the prime editor comprises an engineered Moloney murine leukemia virus (“M-MLV”) reverse transcriptase (“RT”) fused to a Cas-H840A nickase (called “PE2”). In some embodiments, the prime editor comprises an engineered M-MLV RT fused to a Cas9-H840A nickase. In some embodiments, the prime editor comprises an engineered M-MLV RT fused to a Streptococcus pyogenes Cas9 (spCas9)-H840A nickase. PE modifications include increased PAM flexibility to increase the utility of PE2 editing, expanding the coverage of targetable pathogenic variants in the Clin Var database that can now be prime edited to 94.4%.

In some embodiments, the prime editing system further comprises a prime editing guide RNA (“pegRNA”). In some embodiments, the cargo comprises a pegRNA or a polynucleotide encoding a pegRNA.

pegRNAs may be designed and synthesized using methods, software, and commercial sources which are well known to those having ordinary skill in the art such that guide RNAs for any given naspDBP or prime editor may be obtained without undue experimentation.

Reference may be made to the following references providing information and tools for the design, synthesis, modification, and structural configuration of pegRNAs, and guide RNAs in general: (1) Mohr S E, Hu Y, Ewen-Campen B, Housden B E, Viswanatha R, Perrimon N. CRISPR guide RNA design for research applications. FEBS J. 2016 September; 283(17):3232-8. doi: 10.1111/febs.13777. Epub 2016 Jun. 22. PMID: 27276584; PMCID: PMC5014588; (2) Hoberecht L, Perampalam P, Lun A, Fortin J P. A comprehensive Bioconductor ecosystem for the design of CRISPR guide RNAs across nucleases and technologies. Nat Commun. 2022 Nov. 2; 13(1):6568. doi: 10.1038/s41467-022-34320-7. PMID: 36323688; PMCID: PMC9630310; (3) Cram D, Kulkarni M, Buchwaldt M, Rajagopalan N, Bhowmik P, Rozwadowski K, Parkin I A P, Sharpe A G, Kagale S. WheatCRISPR: a web-based guide RNA design tool for CRISPR/Cas9-mediated genome editing in wheat. BMC Plant Biol. 2019 Nov. 6; 19(1):474. doi: 10.1186/s12870-019-2097-z. PMID: 31694550; PMCID: PMC6836449; (4) Pliatsika V, Rigoutsos I. “Off-Spotter”: very fast and exhaustive enumeration of genomic lookalikes for designing CRISPR/Cas guide RNAs. Biol Direct. 2015 Jan. 29; 10:4. doi: 10.1186/s13062-015-0035-z. PMID: 25630343; PMCID: PMC4326336; (5) Hoof J B, Nødvig CS, Mortensen U H. Genome Editing: CRISPR-Cas9. Methods Mol Biol. 2018; 1775:119-132. doi: 10.1007/978-1-4939-7804-5_11. PMID: 29876814; (6) Labun K, Krause M, Torres Cleuren Y, Valen E. CRISPR Genome Editing Made Easy Through the CHOPCHOP Website. Curr Protoc. 2021 April; 1(4):e46. doi: 10.1002/cpz1.46. PMID: 33905612; (7) Lee C M, Davis T H, Bao G. Examination of CRISPR/Cas9 design tools and the effect of target site accessibility on Cas9 activity. Exp Physiol. 2018 Apr. 1; 103(4):456-460. doi: 10.1113/EP086043. Epub 2017 Apr. 12. PMID: 28303677; PMCID: PMC7266697; (8) Ma S, Lv J, Feng Z, Rong Z, Lin Y. Get ready for the CRISPR/Cas system: A beginner's guide to the engineering and design of guide RNAs. J Gene Med. 2021 November; 23(11):e3377. doi: 10.1002/jgm.3377. Epub 2021 Jul. 28. PMID: 34270141; (9) Hiranniramol K, Chen Y, Wang X. CRISPR/Cas9 Guide RNA Design Rules for Predicting Activity. Methods Mol Biol. 2020; 2115:351-364. doi: 10.1007/978-1-0716-0290-4_19. PMID: 32006410; (10) Wiles M V, Qin W, Cheng A W, Wang H. CRISPR-Cas9-mediated genome editing and guide RNA design. Mamm Genome. 2015 October; 26(9-10):501-10. doi: 10.1007/s00335-015-9565-z. Epub 2015 May 20. PMID: 25991564; PMCID: PMC4602062; (11) Creutzburg S C A, Wu W Y, Mohanraju P, Swartjes T, Alkan F, Gorodkin J, Staals R H J, van der Oost J. Good guide, bad guide: spacer sequence-dependent cleavage efficiency of Cas12a. Nucleic Acids Res. 2020 Apr. 6; 48(6):3228-3243. doi: 10.1093/nar/gkz1240. PMID: 31989168; PMCID: PMC7102956; (12) Heigwer F, Boutros M. Cloud-Based Design of Short Guide RNA (sgRNA) Libraries for CRISPR Experiments. Methods Mol Biol. 2021; 2162:3-22. doi: 10.1007/978-1-0716-0687-2_1. PMID: 32926374; (13) Dronina J, Samukaite-Bubniene U, Ramanavicius A. Towards application of CRISPR-Cas12a in the design of modern viral DNA detection tools (Review). J Nanobiotechnology. 2022 Jan. 21; 20(1):41. doi: 10.1186/s12951-022-01246-7. PMID: 35062978; PMCID: PMC8777428; (14) Krysler A R, Cromwell C R, Tu T, Jovel J, Hubbard B P. Guide RNAs containing universal bases enable Cas9/Cas12a recognition of polymorphic sequences. Nat Commun. 2022 Mar. 25; 13(1):1617. doi: 10.1038/s41467-022-29202-x. PMID: 35338140; PMCID: PMC8956631; (15) Shin H R, Kweon J, Kim Y. Gene Manipulation Using Fusion Guide RNAs for Cas9 and Cas12a. Methods Mol Biol. 2021; 2162:185-193. doi: 10.1007/978-1-0716-0687-2_10. PMID: 32926383; (16) Schubert M S, Thommandru B, Woodley J, Turk R, Yan S, Kurgan G, McNeill M S, Rettig G R. Optimized design parameters for CRISPR Cas9 and Cas12a homology-directed repair. Sci Rep. 2021 Sep. 30; 11(1):19482. doi: 10.1038/s41598-021-98965-y. PMID: 34593942; PMCID: PMC8484621; (17) Crone M A, MacDonald JT, Freemont P S, Siciliano V. gDesigner: computational design of synthetic gRNAs for Cas12a-based transcriptional repression in mammalian cells. NPJ Syst Biol Appl. 2022 Sep. 16; 8(1):34. doi: 10.1038/s41540-022-00241-w. PMID: 36114193; PMCID: PMC9481559; (18) Konstantakos V, Nentidis A, Krithara A, Paliouras G. CRISPR-Cas9 gRNA efficiency prediction: an overview of predictive tools and the role of deep learning. Nucleic Acids Res. 2022 Apr. 22; 50(7):3616-3637. doi: 10.1093/nar/gkac192. PMID: 35349718; PMCID: PMC9023298; (19) Wang J, Zhang X, Cheng L, Luo Y. An overview and metanalysis of machine and deep learning-based CRISPR gRNA design tools. RNA Biol. 2020 January; 17(1):13-22. doi: 10.1080/15476286.2019.1669406. Epub 2019 Sep. 27. PMID: 31533522; PMCID: PMC6948960; and (20) Cram D, Kulkarni M, Buchwaldt M, Rajagopalan N, Bhowmik P, Rozwadowski K, Parkin I A P, Sharpe A G, Kagale S. WheatCRISPR: a web-based guide RNA design tool for CRISPR/Cas9-mediated genome editing in wheat. BMC Plant Biol. 2019 Nov. 6; 19(1):474. doi: 10.1186/s12870-019-2097-z. PMID: 31694550; PMCID: PMC6836449; each of which are incorporated herein by reference in their entireties.

In the specific case of prime editing, in particular, further reference may be made to the following references providing information and tools for the design, synthesis, modification, and structural configuration of pegRNAs: (1) Hsu J Y, Grünewald J, Szalay R, Shih J, Anzalone A V, Lam K C, Shen M W, Petri K, Liu D R, Joung J K, Pinello L. PrimeDesign software for rapid and simplified design of prime editing guide RNAs. Nat Commun. 2021 Feb. 15; 12(1):1034. doi: 10.1038/s41467-021-21337-7. PMID: 33589617; PMCID: PMC7884779; (2) Li Y, Chen J, Tsai S Q, Cheng Y. Easy-Prime: a machine learning-based prime editor design tool. Genome Biol. 2021 Aug. 19; 22(1):235. doi: 10.1186/s13059-021-02458-0. PMID: 34412673; PMCID: PMC8377858; (3) Zhang W, Petri K, Ma J, Lee H, Tsai C L, Joung J K, Yeh J J. Enhancing CRISPR prime editing by reducing misfolded pegRNA interactions. bioRxiv [Preprint]. 2023 Aug. 15:2023.08.14.553324. doi: 10.1101/2023.08.14.553324. PMID: 37645936; PMCID: PMC10462064; (4) Jin S, Lin Q, Gao Q, Gao C. Optimized prime editing in monocot plants using PlantPegDesigner and engineered plant prime editors (ePPEs). Nat Protoc. 2023 March; 18(3):831-853. doi: 10.1038/s41596-022-00773-9. Epub 2022 Nov. 25. PMID: 36434096; (5) Lin Q, Jin S, Zong Y, Yu H, Zhu Z, Liu G, Kou L, Wang Y, Qiu J L, Li J, Gao C. High-efficiency prime editing with optimized, paired pegRNAs in plants. Nat Biotechnol. 2021 August; 39(8):923-927. doi: 10.1038/s41587-021-00868-w. Epub 2021 Mar. 25. PMID: 33767395; (6) Standage-Beier K, Tekel S J, Brafman D A, Wang X. Prime Editing Guide RNA Design Automation Using PINE-CONE. ACS Synth Biol. 2021 Feb. 19; 10(2):422-427. doi: 10.1021/acssynbio.0c00445. Epub 2021 Jan. 19. PMID: 33464043; PMCID: PMC7901017; (7) Zhang W, Petri K, Ma J, Lee H, Tsai C L, Joung J K, Yeh J J. Enhancing CRISPR prime editing by reducing misfolded pegRNA interactions. bioRxiv [Preprint]. 2023 Aug. 15:2023.08.14.553324. doi: 10.1101/2023.08.14.553324. PMID: 37645936; PMCID: PMC10462064; (8) Chow R D, Chen J S, Shen J, Chen S. A web tool for the design of prime-editing guide RNAs. Nat Biomed Eng. 2021 February; 5(2):190-194. doi: 10.1038/s41551-020-00622-8. Epub 2020 Sep. 28. PMID: 32989284; PMCID: PMC7882013; each of which are incorporated herein by reference in their entireties.

Reference may also be made to the following commercial vendors which synthesize pegRNAs for prime editing applications and provide various tools and instruction for the ordering, design, synthesis, modification, and structural configuration of pegRNAs: GENSCRIPT, SYNTHEGO, TAKARA BIO, INTEGRATED DNA TECHNOLOGIES, LC SCIENCES, HORIZON DISCOVERY; SIGMA-ALDRICH; ORIGENE, and TWIST BIOSCIENCES, among others.

In addition, pegRNAs may be modified with chemical modifications and/or structural modifications for enhancing various properties thereof, including specificity, stability, and limiting off-target activity. One of ordinary skill in the art will be able to modify a guide RNA with any known modification without undue experimentation. Guide modifications are discussed in the following references: (1) Ke Y, Ghalandari B, Huang S, Li S, Huang C, Zhi X, Cui D, Ding X. 2′-O-Methyl modified guide RNA promotes the single nucleotide polymorphism (SNP) discrimination ability of CRISPR-Cas12a systems. Chem Sci. 2022 Feb. 1; 13(7):2050-2061. doi: 10.1039/d1sc06832f. PMID: 35308857; PMCID: PMC8848812; (2) Allen D, Rosenberg M, Hendel A. Using Synthetically Engineered Guide RNAs to Enhance CRISPR Genome Editing Systems in Mammalian Cells. Front Genome Ed. 2021 Jan. 28; 2:617910. doi: 10.3389/fgeed.2020.617910. PMID: 34713240; PMCID: PMC8525374; (3) Basila M, Kelley M L, Smith A V B. Minimal 2′-O-methyl phosphorothioate linkage modification pattern of synthetic guide RNAs for increased stability and efficient CRISPR-Cas9 gene editing avoiding cellular toxicity. PLOS One. 2017 Nov. 27; 12(11):e0188593. doi: 10.1371/journal.pone.0188593. PMID: 29176845; PMCID: PMC5703482; (4) Sakovina L, Vokhtantsev I, Vorobyeva M, Vorobyev P, Novopashina D. Improving Stability and Specificity of CRISPR/Cas9 System by Selective Modification of Guide RNAs with 2′-fluoro and Locked Nucleic Acid Nucleotides. Int J Mol Sci. 2022 Nov. 3; 23(21):13460. doi: 10.3390/ijms232113460. PMID: 36362256; PMCID: PMC9655745; (5) Shapiro J, Tovin A, Iancu O, Allen D, Hendel A. Chemical Modification of Guide RNAs for Improved CRISPR Activity in CD34+Human Hematopoietic Stem and Progenitor Cells. Methods Mol Biol. 2021; 2162:37-48. doi: 10.1007/978-1-0716-0687-2_3. PMID: 32926376; (6) Filippova J, Matveeva A, Zhuravlev E, Stepanov G. Guide RNA modification as a way to improve CRISPR/Cas9-based genome-editing systems. Biochimie. 2019 December; 167:49-60. doi: 10.1016/j.biochi.2019.09.003. Epub 2019 Sep. 4. PMID: 31493470; (7) Hendel A, Bak R O, Clark J T, Kennedy A B, Ryan D E, Roy S, Steinfeld I, Lunstad B D, Kaiser R J, Wilkens A B, Bacchetta R, Tsalenko A, Dellinger D, Bruhn L, Porteus M H. Chemically modified guide RNAs enhance CRISPR-Cas genome editing in human primary cells. Nat Biotechnol. 2015 September; 33(9):985-989. doi: 10.1038/nbt.3290. Epub 2015 Jun. 29. PMID: 26121415; PMCID: PMC4729442; (8) Ryan D E, Taussig D, Steinfeld I, Phadnis S M, Lunstad B D, Singh M, Vuong X, Okochi K D, McCaffrey R, Olesiak M, Roy S, Yung C W, Curry B, Sampson J R, Bruhn L, Dellinger D J. Improving CRISPR-Cas specificity with chemical modifications in single-guide RNAs. Nucleic Acids Res. 2018 Jan. 25; 46(2):792-803. doi: 10.1093/nar/gkx1199. Erratum in: Nucleic Acids Res. 2022 Mar. 21; 50(5):2986. PMID: 29216382; PMCID: PMC5778453; (9) Palumbo C M, Gutierrez-Bujari J M, O'Geen H, Segal D J, Beal P A. Versatile 3′ Functionalization of CRISPR Single Guide RNA. Chembiochem. 2020 Jun. 2; 21(11):1633-1640. doi: 10.1002/cbic.201900736. Epub 2020 Mar. 5. PMID: 31943634; PMCID: PMC7323579; (10) Mullally G, van Aelst K, Naqvi M M, Diffin F M, Karvelis T, Gasiunas G, Siksnys V, Szczelkun M D. 5′ modifications to CRISPR-Cas9 gRNA can change the dynamics and size of R-loops and inhibit DNA cleavage. Nucleic Acids Res. 2020 Jul. 9; 48(12):6811-6823. doi: 10.1093/nar/gkaa477. PMID: 32496535; PMCID: PMC7337959; (12) Lu S, Zhang Y, Yin H. Chimeric DNA-RNA Guide RNA Designs. Methods Mol Biol. 2021; 2162:79-85. doi: 10.1007/978-1-0716-0687-2_6. PMID: 32926379; each of which are incorporated by reference herein in their entireties.

In the specific case of prime editing, pegRNAs may be further modified with chemical modifications and/or structural modifications for enhancing various properties thereof, including specificity, stability, and limiting off-target activity. One of ordinary skill in the art will be able to modify a pegRNA for prime editing with any known modification without undue experimentation. pegRNA modifications are discussed in the following references: (1) Nelson J W, Randolph P B, Shen S P, Everette K A, Chen P J, Anzalone A V, An M, Newby G A, Chen J C, Hsu A, Liu D R. Engineered pegRNAs improve prime editing efficiency. Nat Biotechnol. 2022 March; 40(3):402-410. doi: 10.1038/s41587-021-01039-7. Epub 2021 Oct. 4. Erratum in: Nat Biotechnol. 2021 Dec. 8; PMID: 34608327; PMCID: PMC8930418; (2) Liu B, Dong X, Cheng H, Zheng C, Chen Z, Rodríguez TC, Liang S Q, Xue W, Sontheimer E J. A split prime editor with untethered reverse transcriptase and circular RNA template. Nat Biotechnol. 2022 September; 40(9):1388-1393. doi: 10.1038/s41587-022-01255-9. Epub 2022 Apr. 4. PMID: 35379962; each of which are incorporated by reference herein in their entireties. In some embodiments, the prime editing system further comprises a second guide RNA targeting the complementary strand, allowing the Cas9 nickase to also nick the non-edited strand (called “PE3”), which biases mismatch DNA repair in favor of the edited sequence. In some embodiments, the second guide RNA is designed to recognize the complementary strand of DNA only after the PE3 edit has occurred (called “PE3b”), which reduces indel formation.

In some embodiments, the prime editing system comprises an uracil glycosylase inhibitor. In some embodiments, the prime editing system comprises a Cas9 protein fused to an uracil glycosylase inhibitor. In some embodiments, the cargo comprises an uracil glycosylase inhibitor or a polynucleotide encoding an uracil glycosylase inhibitor. In some embodiments, the cargo comprises a Cas9 protein fused to an uracil glycosylase inhibitor or a polynucleotide encoding a Cas9 protein fused to an uracil glycosylase inhibitor.

Any of the above prime editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

In embodiments, the reverse transcriptase component of the prime editor can be any reverse transcriptase known in the art, or any variant thereof, such as those described in the above published prime editing application or in the scientific literature, such as in: (1) Gao Z, Ravendran S, Mikkelsen NS, Haldrup J, Cai H, Ding X, Paludan S R, Thomsen M K, Mikkelsen J G, Bak R O. A truncated reverse transcriptase enhances prime editing by split AAV vectors. Mol Ther. 2022 Sep. 7; 30(9):2942-2951. doi: 10.1016/j.ymthe.2022.07.001. Epub 2022 Jul. 8. PMID: 35808824; PMCID: PMC9481986; (2) Lan T, Chen H, Tang C, Wei Y, Liu Y, Zhou J, Zhuang Z, Zhang Q, Chen M, Zhou X, Chi Y, Wang J, He Y, Lai L, Zou Q. Mini-PE, a prime editor with compact Cas9 and truncated reverse transcriptase. Mol Ther Nucleic Acids. 2023 Aug. 18; 33:890-897. doi: 10.1016/j.omtn.2023.08.018. PMID: 37680986; PMCID: PMC10480570; (3) or available biological sequence databases, all of which are incorporated herein by reference.

In addition, the reverse transcriptase may be a retron reverse transcriptase (retron RT), such as any of those described in: (1) U.S. patent application Ser. No. 18/087,673; (2) International PCT Application No. PCT/US2023/061038; (3) International Application No. PCT/US2023/072872; (4) Mestre et al., Nucleic Acids Research, Volume 48, Issue 22, 16 Dec. 2020, Pages 12632-12647; (5) Mestre et al., UG/Abi: “A Highly Diverse Family of Prokaryotic Reverse Transcriptases Associated With Defense Functions,” doi.org/10.1101/2021.12.02.470933; (6) International Application No. PCT/US2023/016262; (7) International Application No. PCT/US2023/016263; (8) International Application No. PCT/US23/72799; (9) International Application NO. PCT/US2022/079220; (10) International Application No. PCT/US2023/016317; and (10) may particularly be any retron selected from Table A of International Application No. PCT/US2023/072872, or any amino acid sequence having at having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A of International Application No. PCT/US2023/072872. The contents of each of the documents in this paragraph are incorporated herein by reference in their entireties.

### Retron Editors

In still other embodiments, the herein disclosed LNPs may be used to encapsulate and deliver a retron editing system. A retron editing system in various embodiments may comprise (a) a retron reverse transcriptase, or a nucleic acid molecule encoding a retron reverse transcriptase, (b) a retron ncRNA (or a nucleic acid molecule encoding same) comprising a modified msd region to include a sequence that is reverse transcribed to form a single strand template DNA sequence (RT-DNA), (c) a nucleic acid programmable nuclease (e.g., a CRISPR Cas9 or Cas12a), and (d) a guide RNA to target the nuclease to a desired target site.

Retrons are defined by their unique ability to produce an unusual satellite DNA known as msDNA (multicopy single-stranded DNA). DNA encoding retrons includes a reverse trancriptase (RT)-coding gene (ret) and a nucleic acid sequence encoding the non-coding RNA (ncRNA), which contains two contiguous and inverted non-coding sequences referred to as the msr and msd. The ret gene and the non-coding RNA (including the msr and msd) are transcribed as a single RNA transcript, which becomes folded into a specific secondary structure following post-transcriptional processing. Once translated, the RT binds the RNA template downstream from the msd locus, initiating reverse transcription of the RNA towards its 5′ end, assisted by the 2′OH group present in a conserved branching guanosine residue that acts as a primer. Reverse transcription halts before reaching the msr locus, and the resulting DNA, the msDNA, remains covalently attached to the RNA template via a 2′-5′ phosphodiester bond and base-pairing between the 3′ ends of the msDNA and the RNA template. The external regions, at the 5′ and 3′ ends of the msd/msr transcript (a1 and a2, respectively) are complementary and can hybridize, leaving the structures located in the msr and msd regions in internal positions. The msr locus, which is not reverse transcribed, forms one to three short stem-loops of variable size, ranging from 3 to 10 base pairs, whereas the msd locus folds into a single/double long hairpin with a highly variable long stem of 10-50 bp in length that is also present in the final msDNA form.

It has recently been reported that retrons may be utilized as a means to provide donor DNA template for HDR-dependent genome editing (e.g., see Lopez et al., “Precise genome editing across kingdoms of life using retron-derived DNA,” Nature Chemical Biology, Dec. 12, 2021, 18, pages 199-206 (2022)), however, producing sufficient levels of donor DNA template intracellularly to sufficiently support efficient HDR-dependent editing remains a significant challenge.

Retrons have previously been described in the scientific literature, including in the context of retron editing. For example, retrons have been described in the following references, each of which are incorporated herein by reference:

| Title | Pub. | Journal | Author/s | V | Ps |
|---|---|---|---|---|---|
| Recording gene expression | Jul. 27, 2022 | Nature | Bhattarai- | 608 | 217-225 |
| order in DNA by CRISPR | | | Kline; etal. | | |
| addition of retron barcodes. | | | | | |
| Retrons Display Genome | Jun. 1, 2021 | Genetic | | 41 | 15-15 |
| Editing Strengths Even | | Engineering & | | | |
| CRISPR Might Envy | | Biotechnology | | | |
| | | News | | | |
| Retron reverse transcriptase | Mar. 16, 2022 | Nucleic acids | Palka; et al. | 50 | 3490-3504 |
| termination and phage | | research | | | |
| defense are dependent on host | | | | | |
| RNase H1. | | | | | |
| Retrons: Complementing | Aug. 1, 2020 | The CRISPR | Maxwell | 3 | 226-227 |
| CRISPR in Phage Defense. | | journal | | | |
| Systematic prediction of | Dec. 4, 2020 | Nucleic acids | Rodríguez | 48 | 12632-12647 |
| genes functionally associated | | research | Mestre; et al. | | |
| with bacterial retrons and | | | | | |
| classification of the encoded | | | | | |
| tripartite systems. | | | | | |
| Precise genome editing | Aug. 17, 2021 | Protein & cell | Kong; et al. | 12 | 899-902 |
| without exogenous donor | | | | | |
| DNA via retron editing | | | | | |
| system in human cells. | | | | | |
| Precise genome editing | Dec. 23, 2021 | Nature chemical | Lopez; et al. | 18 | 199-206 |
| across kingdoms of life using | | biology | | | |
| retron-derived DNA. | | | | | |
| Prokaryotic reverse | May 13, 2021 | FEMS | González- | 45 | |
| transcriptases: from | | microbiology | Delgado, et al. | | |
| retroelements to specialized | | reviews | | | |
| defense systems | | | | | |
| A function for retrons. | Nov. 17, 2020 | Nature reviews. | Grant Otto | 19 | 3-3 |
| | | Microbiology | | | |
| Retrons and their applications | Oct. 10, 2019 | Nucleic acids | Simon, et al. | 47 | 11007-11019 |
| in genome engineering. | | research | | | |
| High-efficiency retron- | Jan. 1, 2022 | Synthetic | Jiang, et al. | 7 | |
| mediated single-stranded | | biology | | | |
| DNA production in plants. | | (Oxford, | | | |
| | | England) | | | |
| Bacterial retrons enable | Jan. 24, 2022 | The CRISPR | Zhao B, et al. | 5 | 31-39 |
| precise gene editing in human | | journal | | | |
| cells | | | | | |
| Like CRISPR, mystery gene | Nov. 20, 2020 | Science (New | Pennisi | 370 | 898-899 |
| editor began as a virus | | York, N.Y.) | | | |
| fighter. | | | | | |
| Bacterial Retrons Function In | Nov. 5, 2020 | Cell | Millman, et al. | 183 | 1551-1561 |
| Anti-Phage Defense. | | | | | |
| Multiplex generation, | May 4, 2020 | ACS synthetic | Lim, et al. | 9 | 1003-1009 |
| tracking, and functional | | biology | | | |
| screening of substitution | | | | | |
| mutants using a crispr/retron | | | | | |
| system | | | | | |
| Multiple origins of reverse | Jul. 11, 2019 | RNA biology | Toro, et al. | 16 | 1486-1493 |
| transcriptases linked to | | | | | |
| CRISPR-Cas systems. | | | | | |
| Retroelement-Based Genome | Oct. 26, 2018 | ACS synthetic | Simon, et al. | 7 | 2600-2611 |
| Editing and Evolution. | | biology | | | |

In addition, retrons have previously been described in the patent literature, including in the context of retron editing. For example, retrons have been described in the following references, each of which are incorporated herein by reference:

| Publication No. | TITLE |
|---|---|
| WO 2024/044723 | ENGINEERED RETRONS AND METHOD OF USE |
| U.S. Pat. No. 11,866,728 | ENGINEERED RETRONS AND METHOD OF USE |
| WO2023/141602 | ENGINEERED RETRONS AND METHOD OF USE |
| US 2020/0115706 A1 | METHOD OF RECORDING MULTIPLEXED BIOLOGICAL |
| | INFORMATION INTO A CRISPR ARRAY USING A RETRON |
| EP 3510151 A4 | HIGH-THROUGHPUT PRECISION GENOME EDITING |
| US 2019/0330619 A1 | HIGH-THROUGHPUT PRECISION GENOME EDITING |
| EP 3510151 A1 | HIGH-THROUGHPUT PRECISION GENOME EDITING |
| WO 2018/191525 A1 | METHOD OF RECORDING MULTIPLEXED BIOLOGICAL |
| | INFORMATION INTO A CRISPR ARRAY USING A RETRON |
| US 2018/0127759 A1 | DYNAMIC GENOME ENGINEERING |
| WO 2018/049168 A1 | HIGH-THROUGHPUT PRECISION GENOME EDITING |
| US 2017/0204399 A1 | GENOMICALLY-ENCODED MEMORY IN LIVE CELLS |
| EP 3180430 A1 | GENOMICALLY-ENCODED MEMORY IN LIVE CELLS |
| CA 2488328 C | RETRONS FOR GENE TARGETING |
| WO 2016/025719 A1 | GENOMICALLY-ENCODED MEMORY IN LIVE CELLS |
| U.S. Pat. No. 8,932,860 B2 | RETRONS FOR GENE TARGETING |
| EP 1517992 B1 | RETRONS FOR GENE TARGETING |
| AU 2003/233734 C1 | RETRONS FOR GENE TARGETING |
| AU 2003/233734 B2 | RETRONS FOR GENE TARGETING |
| US 2009/0123991 A1 | RETRONS FOR GENE TARGETING |
| US 2005/0250207 A1 | RETRONS FOR GENE TARGETING |
| EP 1517992 A2 | RETRONS FOR GENE TARGETING |
| WO 2003/104470 A3 | RETRONS FOR GENE TARGETING |
| AU 2003/233734 A1 | RETRONS FOR GENE TARGETING |
| CA 2488328 A1 | RETRONS FOR GENE TARGETING |
| WO 2003/104470 A2 | RETRONS FOR GENE TARGETING |

In some embodiments, the LNP-based retron editing system can be used for genome editing a desired site. A retron is engineered with a heterologous nucleic acid sequence encoding a donor polynucleotide (“template or donor nucleotide sequence” or “template DNA”) suitable for use with nuclease genome editing system. The nuclease is designed to specifically target a location proximal to the desired edit (the nuclease should be designed such that it will not cut the target once the edit is properly installed). The nuclease (e.g., CAS or non-CAS) is linked to the retron, either by direct fusion to the RT or by fusion of the msDNA to the gRNA (only applicable for RNA-guided nucleases). A heterologous nucleic acid sequence is inserted into the retron msd.

In some embodiments, the heterologous nucleic acid sequence has 10-100 or more bp of homologous nucleic acid sequence to the genome on both sides of the desired edit. The desired edit (insertion, deletion, or mutation) is in between the homologous sequence.

In some embodiments, donor polynucleotides comprise a sequence comprising an intended genome edit flanked by a pair of homology arms responsible for targeting the donor polynucleotide to the target locus to be edited in a cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relate to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit can be integrated into the genomic DNA by HDR at the genomic target locus recognized (i.e., having sufficient complementary for hybridization) by the 5′ and 3′ homology arms.

In some embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In some embodiments, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g. 10 nucleotides or more, 15 nucleotides or more, 20 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another. However, in some instances the 5′ and 3′ homology arms are not necessarily equal in length to one another. For example, one homology arm may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g. one may be 40% shorter or more, 50% shorter or more, sometimes 60% shorter or more, 70% shorter or more, 80% shorter or more, 90% shorter or more, or 95% shorter or more than the other homology arm.

The donor polynucleotide may be used in combination with an RNA-guided nuclease, which is targeted to a particular genomic sequence (i.e., genomic target sequence to be modified) by a guide RNA. A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to the sequence of a minor allele to target the nuclease-gRNA complex to the site of a mutation. The mutation may comprise an insertion, a deletion, or a substitution. For example, the mutation may include a single nucleotide variation, gene fusion, translocation, inversion, duplication, frameshift, missense, nonsense, or other mutation associated with a phenotype or disease of interest. The targeted minor allele may be a common genetic variant or a rare genetic variant. In some embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to remove the mutation from a gene. Alternatively, the gRNA can be designed with a sequence complementary to the sequence of a major or wild-type allele to target the nuclease-gRNA complex to the allele for the purpose of genome editing to introduces a mutation into a gene in the genomic DNA of the cell, such as an insertion, deletion, or substitution. Such genetically modified cells can be used, for example, to alter phenotype, confer new properties, or produce disease models for drug screening.

In some embodiments, the RNA-guided nuclease used for genome modification is a clustered regularly interspersed short palindromic repeats (CRISPR) system Cas nuclease. Any RNA-guided Cas nuclease capable of catalyzing site-directed cleavage of DNA to allow integration of donor polynucleotides by the HDR mechanism can be used in genome editing, including CRISPR system Class 1, Type I, II, or III Cas nucleases; Class 2, Type II nuclease (such as Cas9); a Class 2, Type V nuclease (such as Cpf1), or a Class 2, Type VI nuclease (such as C2c2). Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), CasIO, Cas1Od, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cu1966, and homologs or modified versions thereof.

In some embodiments, a Class 1, type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP 002989955, WP_038434062, WP_011528583); Campylobacter jejuni (WP_022552435, YP 002344900), Campylobacter coli (WP 060786116); Campylobacter fetus (WP 059434633); Corynebacterium ulcerans (NC_015683, NC_017317); Corynebacterium diphtheria (NC_016782, NC_016786); Enterococcus faecalis (WP 033919308); Spiroplasma syrphidicola (NC 021284); Prevotella intermedia (NC 017861); Spiroplasma taiwanense (NC 021846); Streptococcus iniae (NC 021314); Belliella baltica (NC 018010); Psychroflexus torquisl (NC O 18721); Streptococcus thermophilus (YP 820832), Streptococcus mutans (WP 061046374, WP 024786433); Listeria innocua (NP 472073); Listeria monocytogenes (WP 061665472); Legionella pneumophila (WP 062726656); Staphylococcus aureus (WP_001573634); Francisella tularensis (WP_032729892, WP_014548420), Enterococcus faecalis (WP 033919308); Lactobacillus rhamnosus (WP 048482595, WP_032965177); and Neisseria meningitidis (WP_061704949, YP_002342100); all of which sequences (as entered by the date of filing of this application) are herein incorporated by reference in their entireties. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacterid. 198(5):797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The genomic target site will typically comprise a nucleotide sequence that is complementary to the gRNA and may further comprise a protospacer adjacent motif (PAM). In some embodiments, the target site comprises 20-30 base pairs in addition to a 3 or more base pair PAM. Typically, the first nucleotide of a PAM can be any nucleotide, while the two or more other nucleotides will depend on the specific Cas9 protein that is chosen. Exemplary PAM sequences are known to those of skill in the art and include, without limitation, NNG, NGN, NAG, and NGG, wherein N represents any nucleotide. In some embodiments, the allele targeted by a gRNA comprises a mutation that creates a PAM within the allele, wherein the PAM promotes binding of the Cas9-gRNA complex to the allele.

In some embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, or Cas12a) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

C2c1 (Cas12b) is another class II CRISPR/Cas system RNA-guided nuclease that may be used. C2c1, similarly to Cas9, depends on both a crRNA and tracrRNA for guidance to target sites. See, e.g., Shmakov et al. (2015) Mol Cell. 60(3):385-397, Zhang et al. (2017) Front Plant Sci. 8:177; herein incorporated by reference.

In yet another embodiment, an engineered RNA-guided FokI nuclease may be used. RNA-guided FokI nucleases comprise fusions of inactive Cas9 (dCas9) and the FokI endonuclease (FokI-dCas9), wherein the dCas9 portion confers guide RNA-dependent targeting on FokI. For a description of engineered RNA-guided Fold nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

In other embodiments, any other Cas enzymes and variants described in other sections of the application (all incorporated herein) can be used similarly.

In some embodiments, the RNA-guided nuclease is provided in the form of a protein, optionally where the nuclease is complexed with a gRNA to form a ribonucleoprotein (RNP) complex. In some embodiments, the RNA-guided nuclease is provided by a nucleic acid encoding the RNA-guided nuclease, such as an RNA (e.g., messenger RNA) or DNA (expression vector). In some embodiments, the RNA-guided nuclease and the gRNA are both provided by vectors, such as the vectors and the vector system described in other parts of the application (all incorporated herein by reference). Both can be expressed by a single vector or separately on different vectors. The vectors encoding the RNA-guided nuclease and gRNA may be included in the vector system comprising the engineered retron msr gene, msd gene and ret gene sequences. In some embodiments, the RNA-guided nuclease is fused to the RT and/or the msDNA.

The RNP complex may be administered to a subject or delivered into a cell by methods known in the art, such as those described in U.S. Pat. No. 11,390,884, which is incorporated by reference herein in its entirety. In some embodiments, the endonuclease/gRNA ribonucleoprotein (RNP) complexes are delivered to cells by electroporation. Direct delivery of the RNP complex to a subject or cell eliminates the need for expression from nucleic acids (e.g., transfection of plasmids encoding Cas9 and gRNA). It also eliminates unwanted integration of DNA segments derived from nucleic acid delivery (e.g., transfection of plasmids encoding Cas9 and gRNA). An endonuclease/gRNA ribonucleoprotein (RNP) complex usually is formed prior to administration.

Codon usage may be optimized to further improve production of an RNA-guided nuclease and/or reverse transcriptase (RT) in a particular cell or organism. For example, a nucleic acid encoding an RNA-guided nuclease or reverse transcriptase can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease or reverse transcriptase is introduced into cells, the protein can be transiently, conditionally, or constitutively expressed in the cell.

In some embodiments, the engineered retron used for genome editing with nuclease genome editing systems can further include accessory or enhancer proteins for recombination. Examples of recombination enhancers can include nonhomologous end joining (NHEJ) inhibitors (e.g., inhibitor of DNA ligase IV, a KU inhibitor (e.g., KU70 or KU80), a DNA-PKc inhibitor, or an artemis inhibitor) and homologous directed repair (HDR) promoters, or both, that can enhance or improve more precise genome editing and/or the efficiency of homologous recombination. In some embodiments, the recombination accessory or enhancers can comprise C-terminal binding protein interacting protein (CIP), cyclinB2, Rad family members (e.g. Rad50, Rad51, Rad52, etc).

CtIP is a transcription factor containing C2H2 zinc fingers that are involved in early steps of homologous recombination. Mammalian CtIP and its orthologs in other eukaryotes promote the resection of DNA double-strand breaks and are essential for meiotic recombination. HDR may be enhanced by using Cas9 nuclease associated (e.g. fused) to an N-terminal domain of CtIP, an approach that forces CtIP to the cleavage site and increases transgene integration by HDR. In some embodiments, an N-terminal fragment of CtIP, called HE for HDR enhancer, may be sufficient for HDR stimulation and requires the CtIP multimerization domain and CDK phosphorylation sites to be active. HDR stimulation by the Cas9-HE fusion depends on the guide RNA used, and therefore the guide RNA will be designed accordingly.

Using the gene editing system described herein, any target gene or sequence in a host cell can be edited or modified for a desired trait, including but not limited to: Myostatin (e.g., GDF8) to increase muscle growth; Pc POLLED to induce hairlessness; KISS1R to induce bore taint; Dead end protein (dnd) to induce sterility; Nano2 and DDX to induce sterility; CD163 to induce PRRSV resistance; RELA to induce ASFV resilience; CD18 to induce Mannheimia (Pasteurella) haemolytica resilience; NRAMPI to induce tuberculosis resilience; Negative regulators of muscle mass (e.g., Myostatin) to increase muscle mass.

Any of the above retron editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

### TnpB Editors

In other embodiments, the herein disclosed LNPs may be used to encapsulate and deliver a TnpB editing system and/or components thereof. A TnpB editing system in various embodiments may comprise (a) a TnpB protein, or a nucleic acid molecule encoding a TnpB protein, (b) a TnpB guide RNA known as an “reRNA” or “right end RNA”, and optionally one or more additional components, including (c) an effector domain or otherwise accessory protein, and (d) a DNA template (e.g., a DNA donor for HDR-dependent repair at the TnpB-cut target site.

In various embodiments, the TnpB protein can be naturally occurring or the TnpB can be an engineered variant thereof and can be used in various applications, including precision gene editing in cells, tissues, organs, or organisms. The TnpB-based gene editing systems comprise a TnpB polypeptide and a nucleic acid component capable of forming a complex with the TnpB polypeptide which directs the complex to a target nucleotide sequence (e.g., a genomic target sequence such as a disease-associated gene). The TnpB gene editing systems contemplated herein may also be modified with one or more additional effector or accessory functions, such as a nuclease, recombinase, ligase, reverse transcriptase, polymerase, deaminase, etc. to provide additional genome editing functionality. In addition, the TnpB gene editing systems contemplated herein can utilize a nuclease-limited or nuclease-deficiently TnpB variant. Normal TnpB nuclease activity cuts both strands of a target DNA, however, TnpB nickases (having only the ability to cut one of the two strands but not both strands) and nuclease-inactive or “dead” TnpB (which does not cut either strand) may also be used into the TnpB systems described herein, particularly when combined with at least another genome editing functionality, such as a deaminase (for base editing functionality) or a reverse transcriptase (for prime editing functionality). Thus, disclosed herein are TnpB systems that may function as nuclease, nickases, or catalytically inactive polynucleotide binding proteins that can be coupled with other functional domains, such as deaminases, recombinase, ligases, polymerases (e.g., reverse transcriptase), nucleases, or reverse transcriptases.

In one embodiment, the TnpB systems and related compositions may specifically target single-strand or double-strand DNA. In one embodiment, the TnpB system may bind and cleave double-strand DNA. In one embodiment, the TnpB system may bind to double-stranded DNA without introducing a break to either of the strands. In one embodiment, the TnpB polypeptides or nuclease/nucleic acid component complexes may open, disrupting the continuity of one of the two DNA strands, thereby introducing a nick of the double stranded DNA. In an embodiment, and without being bound by theory, the size and configuration of the TnpB systems allows exposure to the non-targeting strand, which may be in single-stranded form, to allow for for the ability to modify, edit, delete or insert polynucleotides on the non-target strand. In an embodiment, this accessibility further allows for enhanced editing outcomes on the target and/or non-target strand, e.g., increased specificity, enhanced editing efficiency.

In one aspect, embodiments disclosed herein are directed to compositions comprising a TnpB and a reRNA capable of forming a complex with the TnpB and directing site-specific binding of the TnpB to a target sequence on a target polynucleotide.

### A. TnpB Polypeptides

Any TnpB polypeptide may be utilized with the compositions described herein. The below description of various TnpBs which can be used in connection with the presently disclose TnpB editing systems is not meant to be limiting in any way. The TnpB editing systems disclosed herein may comprise a canonical or naturally-occurring TnpB, or any ortholog TnpB protein, or any variant TnpB protein—including any naturally occurring variant, mutant, or otherwise engineered version of TnpB—that is known or which can be made or evolved through a directed evolutionary or otherwise mutagenic process. In various embodiments, the TnpB or TnpB variants can have a nickase activity, i.e., only cleave of strand of the target DNA sequence. In other embodiments, the TnpB or TnpB variants have inactive nucleases, i.e., are “dead” TnpB proteins. Other variant TnpB proteins that may be used are those having a smaller molecular weight than the canonical TnpB (e.g., for easier delivery) or having modified amino acid sequences or substitutions.

Examples of TnpB proteins are provided as follows; however, these specific examples are not meant to be limiting. The TnpB editing systems of the present disclosure may use any suitable TnpB protein. In various embodiments, the TnpB editing systems of the present disclosure may include one or more TnpB polypeptides selected from those disclosed in WO 2023/240261A1, or a polypeptide having at least 70%, 75%, 80%, 85%, 90%, 95%, 99%, or 100% sequence identity with one or more of the TnpB polypeptides of WO 2023/240261A1, which is incorporated by reference herein, in its entirety.

In various other embodiments, the TnpB editing systems of the present disclosure may include one or more TnpB polypeptides and reRNAs disclosed in any of the following published applications, or a polypeptide (or reRNA as the case may be) having at least 70%, 75%, 80%, 85%, 90%, 95%, 99%, or 100% sequence identity with one or more of the TnpB polypeptides or reRNAs disclosed therein: US2023/0056577; US2023/0051396 A1; U.S. Pat. No. 11,578,313 B2; US2023/0040216 A1; WO2023/015259 A2; US2023/0032369 A1; US2023/0033866 A1; WO2023/004430 A1; U.S. Pat. No. 11,560,555 B2; WO2023/275601 A1; WO2022/253903 A1; WO2022/248607 A2; US2022/0372525 A1; US2022/0348929 A1; US2022/0348925 A1; U.S. Pat. No. 11,453,866 B2; WO2022/173830 A1; WO2022/174144 A1; WO2022/159892 A1; WO2022/150651 A1; U.S. Pat. No. 11,384,344 B2; WO2022/140572 A1; US2022/0195503 A1; WO2022/098923 A1; WO2022/087494 A1; WO2022/086846 A2; WO2022/076425 A1; WO2022/076890 A1; WO2021/257997 A2; WO2021/247924 A1; US2021/0380956 A1; U.S. Pat. No. 11,180,751 B2; WO2021/188729 A1; WO2021/188286 A2; WO2021/183807 A1; WO2021/159020 A2; US2021/0214697 A1; US2021/0166783 A1; WO2021/050601 A1; EP3009511 B2; US2020/0291395 A1; US2020/0239896 A1; WO2019/178428 A1; US2012/0178668 A1; U.S. Pat. No. 7,608,450 B2; US2004/0091856 A1; US2004/0009477 A1; US2003/0134302 A1; U.S. Pat. No. 6,562,958 B1; and WO1999/051766 A1, each of which are incorporated in their entireties by reference.

In certain example embodiments, the TnpB polypeptides are between 175 and 800 amino acids in size, between 200 and 790 amino acids in size, between 200 and 780 amino acids in size, between 200 and 770 amino acids in size, between 200 and 760 amino acids in size, between 200 and 750 amino acids in size, between 200 and 740 amino acids in size, between 200 and 730 amino acids in size, between 200 and 720 amino acids in size, between 200 and 720 amino acids in size, between 200 and 710 amino acids in size, between 200 and 700 amino acids in size, between 200 and 690 amino acids in size, between 200 and 680 amino acids in size, between 200 and 670 amino acids in size, between 200 and 660 amino acids in size, between 200 and 650 amino acids in size, between 200 and 640 amino acids in size, between 200 and 630 amino acids in size, between 200 and 620 amino acids in size, between 200 and 610 amino acids in size, between 200 and 600 amino acids in size, between 200 and 590 amino acids in size, between 200 and 580 amino acids in size, between 200 and 570 amino acids in size, between 200 and 560 amino acid, between 200 between 550 amino acids, between 200 and 540 amino acids, between 200 and 530 amino acids, between 200 and 520 amino acids, between 200 and 510 amino acids, between 200 and 500 amino acids, between 200 and 490 amino acids, between 200 and 480 amino acids, between 200 and 470 amino acids, between 200 and 460 amino acids, between 200 and 450 amino acids, between 200 and 440 amino acids, between 200 and 430 amino acids, between 200 and 420 amino acids, between 200 and 410 amino acids, between 210 and 500 amino acids, between 220 and 500 amino acids. Between 230 and 500 amino acids, between 240 and 500 amino acids, between 250 and 500 amino acids, between 260 and 500 amino acids, between 270 and 500 amino acids, between 280 and 500 amino acids, between 290 and 500 amino acids, between 300 and 500 amino acids, between 250 and 490 amino acids, between 250 and 480 amino acids, between 250 and 490 amino acids, or between 250 and 600 amino acids. In one embodiment, the TnpB polypeptide is between 300 and 500 amino acids, or between 350 and 450 amino acids.

In one embodiment, the TnpB polypeptides may comprise a modified naturally occurring protein, functional fragment or truncated version thereof, or a non-naturally occurring protein. In one embodiment, the TnpB polypeptide comprises one or more domains originating from other TnpB polypeptides, more particularly originating from different organisms. In one embodiment, the TnpB polypeptides may be designed by in silico approaches. Examples of in silico protein design have been described in the art and are therefore known to a skilled person.

The TnpB polypeptides also encompass homologs or orthologs of TnpB polypeptides whose sequences are specifically described herein (such as the sequences of Table A). The terms “ortholog” and “homolog” are well known in the art. By means of further guidance, a “homolog” of a protein as used herein is a protein of the same species which performs the same or a similar function as the protein it is a homolog of. Homologous proteins may but need not be structurally related, or are only partially structurally related. An “ortholog” of a protein as used herein is a protein of a different species which performs the same or a similar function as the protein it is an orthologue of. Orthologous proteins may be, but may not always be, structurally related or are only partially structurally related. In particular embodiments, the homolog or ortholog of a TnpB polypeptide such as referred to herein has a sequence homology or identity of at least 80%, at least 81%, at least 82%, at least 83%, at least 84% at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with a TnpB polypeptide, more specifically with a TnpB sequence identified in Table A. In particular embodiments, a homolog or ortholog is identified according to its domain structure and/or function. Sequence alignments conducted as described herein, as well as folding studies and domain predictions can aid in the identification of a homolog or ortholog with the structural and functional characteristics identifying TnpB polypeptides, particularly those with conserved residues, including catalytic residues, and domains of TnpB polypeptides.

In one embodiment, the TnpB polypeptide comprises at least at least one RuvC-like nuclease domain. The RuvC domain may comprise conserved catalytic amino acids indicative of the RuvC catalytic residue. In an example embodiment, the RuvC catalytic residue may be referenced relative to D191, E278, and D361 of the TnpB of D. radiodurans or a corresponding amino acid in an aligned sequence. In an aspect, the RuvC domain may comprise multiple subdomains, e.g., RuvC-I, RuvC-II and RuvC-III. The subdomains may be separated by intervening amino acid sequence of the protein.

In one embodiment, examples of the RuvC domain include any polypeptides a structural similarity and/or sequence similarity to a RuvC domain described in the art. In some examples, the RuvC domain may have an amino acid sequence that share at least 50%, at least 55%, at least 60%, at least 65%, at least 70%, at least 75%, at least 80%, at least 85%, at least 90%, at least 95%, at least 99%, or 100% sequence identity with RuvC domains known in the art. One of ordinary skill in the art can modify, substitute, or otherwise alter the activity of the RuvC domain to alter the nuclease activity, such as whether and/or where the nuclease cuts the DNA.

In embodiments, the TnpB polypeptide has a nuclease activity. In one embodiment, the TnpB and the targeting RNA (e.g., the reRNA) can direct sequence-specific nuclease activity. The cleavage may result in a 5′ overhang. The cleavage may occur distal to a target-adjacent motif (TAM), and may occur at the site of the spacer (i.e., the spacer of the reRNA which is complementary to the target sequences) annealing site or 3′ of the target sequence. In an aspect, the TnpB cleaves at multiple positions within and beyond the nucleic acid component annealing site. In an aspect, DNA cleavage occurs 15, 16, 17, 18, 19, 20, 21, 22, 23, 24, 25 or more base pairs distal to the TAM and results in a 5′ overhang. In various embodiments, the TnpB has a nuclease activity against single-stranded DNA. In other embodiments, the TnpB has a nuclease activity against double-stranded DNA.

### B. TnpB Modifications

In various aspects, the present disclosure provides one or more modifications of TnpB comprising TnpB fusions, TnpB mutations to increase sufficiency and/or efficiency and modification of TnpB reRNA. In some embodiments, one or more domains of the TnpB are modified, e.g., wedge domain, corresponding to the β-barrel, REC—helical bundle, RuvC—RuvC domain with the inserted helical hairpin (HH) and the zinc-finger domain (ZnF).

Without intending to be limited to any particular theory, TnpB operates as a homodimer with one DNA molecule and for some orthologs, its ability to form this conformation may be efficacy limiting. Takeda, Satoru N et al. “Structure of the miniature type V-F CRISPR-Cas effector enzyme.” Molecular cell vol. 81,3 (2021): 558-570.e3.

Karvelis et al. demonstrated Deinococcus radiodurans ISDra2 TnpB to be an RNA-directed nuclease guided by RE-derived RNA (reRNA) to cleave DNA next to the 5′ TTGAT transposon associated motif (TAM). Karvelis, T., Druteika, G., Bigclyte, G. et al. Transposon-associated TnpB is a programmable RNA-guided DNA endonuclease. Nature 599, 692-696 (2021).

Without being bound by theory, it is contemplated that TnpB likely operates as a homodimer. Recent studies show that Cas9-Cas9 fusions displayed higher levels of genome modification and a higher proportion of these editing events were precise deletions than are observed for two independent Cas9 nucleases. Bolukbasi, M. F., Liu, P., Luk, K. et al. Orthogonal Cas9-Cas9 chimeras provide a versatile platform for genome editing. Nat Commun 9, 4856 (2018).

Accordingly, in one embodiment, a TnpB is fused to a second TnpB or the like, for example TnpB-TnpB or TnpB-Cas9. Such dual-nuclease formats comprise one TnpB component displaying expanded targeting and/or enhanced specificity and the second TnpB component having nuclease activity. In other preferred embodiments, a TnpB is fused to two or more nuclease proteins.

The TnpB polypeptide may comprise one or more modifications. As used herein, the term “modified” with regard to a TnpB polypeptide generally refers to a TnpB polypeptide having one or more modifications or mutations (including point mutations, truncations, insertions, deletions, chimeras, fusion proteins, etc.) compared to the wild type counterpart from which it is derived (e.g., from a TnpB sequence from Tables B or C). By derived is meant that the derived enzyme is largely based, in the sense of having a high degree of sequence or structural homology with, a wildtype enzyme, but that it has been mutated (modified) in some way as known in the art or as described herein.

The modified proteins, e.g., modified TnpB polypeptide may be catalytically inactive (dead). As used herein, a catalytically inactive or dead nuclease may have reduced, or no nuclease activity compared to a wildtype counterpart nuclease. In some cases, a catalytically inactive or dead nuclease may have nickase activity. In some cases, a catalytically inactive or dead nuclease may not have nickase activity. Such a catalytically inactive or dead nuclease may not make either double-strand or single-strand break on a target polynucleotide but may still bind or otherwise form complex with the target polynucleotide.

In an embodiment, eukaryotic homologues of bacterial TnpB may be utilized in the present disclosure. These TnpB-like proteins, Fanzor 1 and Fanzor 2, while having a shared amino acid motif in their C-terminal half regions, are variable in their N terminal regions.

In one embodiment, the modifications of the TnpB polypeptide may or may not cause an altered functionality. By means of example, modifications which do not result in an altered functionality include for instance codon optimization for expression into a particular host, or providing the nuclease with a particular marker (e.g. for visualization). Modifications with may result in altered functionality may also include mutations, including point mutations, insertions, deletions, truncations (including split nucleases), etc., as well as chimeric nucleases (e.g., comprising domains from different orthologues or homologues) or fusion proteins. Fusion proteins may without limitation include, for instance, fusions with heterologous domains or functional accessory domains (e.g., localization signals, catalytic domains, etc.). In one embodiment, various different modifications may be combined (e.g., a mutated nuclease which is catalytically inactive and which further is fused to a functional domain, such as for instance to induce DNA methylation or another nucleic acid modification, such as including without limitation, a break (e.g. by a different nuclease (domain)), a mutation, a deletion, an insertion, a replacement, a ligation, a digestion, a break or a recombination). As used herein, “altered functionality” includes without limitation an altered specificity (e.g., altered target recognition, increased (e.g., “enhanced” TnpB polypeptide) or decreased specificity, or altered TAM recognition), altered activity (e.g., increased or decreased catalytic activity, including catalytically inactive nucleases or nickases), and/or altered stability (e.g., fusions with destabilization domains).

Examples of all these modifications are known in the art. It will be understood that a “modified” nuclease as referred to herein, and in particular a “modified” TnpB polypeptide or system or complex preferably still has the capacity to interact with or bind to the polynucleic acid (e.g., in complex with the nucleic acid component molecule). Such modified TnpB polypeptide can be combined with the deaminase protein or active domain thereof as described herein.

In one embodiment, an unmodified TnpB polypeptides may have cleavage activity. In one embodiment, the TnpB polypeptides may direct cleavage of one or both nucleic acid (DNA or RNA) strands at the location of or near a target sequence, such as within the target sequence and/or within the complement of the target sequence or at sequences associated with the target sequence. In one embodiment, the TnpB polypeptides may direct cleavage of one or both DNA or RNA strands within about 1, 2, 3, 4, 5, 6, 7, 8, 9, 10, 15, 20, 25, 50, 100, 200, 500, or more base pairs or nucleotides from the first or last nucleotide of a target sequence. In one embodiment, the cleavage may be staggered, i.e., generating sticky ends. In one embodiment, the cleavage is a staggered cut with a 5′ overhang. In one embodiment, the cleavage is a staggered cut with a 5′ overhang of 1 to 5 or up to 10 nucleotides. In particular embodiments, the TnpB polypeptides cleave DNA strands.

In one embodiment, a TnpB polypeptide may be mutated with respect to a corresponding wild-type enzyme (e.g., the TnpB polypeptides of Tables B and C) such that the mutated TnpB lacks the ability to cleave one or both strands of a target polynucleotide containing a target sequence. As a further example, two or more catalytic domains of a TnpB polypeptide (e.g., RuvC) may be mutated to produce a mutated TnpB polypeptide substantially lacking all DNA cleavage activity. In one embodiment, a TnpB polypeptide may be considered to substantially lack all polynucleotide cleavage activity when the polynucleotide cleavage activity of the mutated enzyme is no more than 25%, no more than 10%, no more than 5%, no more than 1%, no more than 0.1%, no more than 0.01% of the nucleic acid cleavage activity of the non-mutated form of the enzyme; an example can be when the nucleic acid cleavage activity of the mutated form is nil or negligible as compared with the non-mutated form.

In one embodiment, the TnpB polypeptide may comprise one or more modifications resulting in enhanced activity and/or specificity, such as including mutating residues that stabilize the targeted or non-targeted strand. In one embodiment, the altered or modified activity of the engineered TnpB polypeptide comprises increased targeting efficiency or decreased off-target binding. In one embodiment, the altered activity of the engineered TnpB polypeptide comprises modified cleavage activity. In one embodiment, the altered activity comprises increased cleavage activity as to the target polynucleotide loci. In one embodiment, the altered activity comprises decreased cleavage activity as to the target polynucleotide loci. In one embodiment, the altered activity comprises decreased cleavage activity as to off-target polynucleotide loci. In one embodiment, the modified nuclease comprises a modification that alters association of the protein with the nucleic acid molecule comprising RNA, or a strand of the target polynucleotide loci, or a strand of off-target polynucleotide loci.

In an aspect of the present disclosure, the engineered TnpB polypeptide comprises a modification that alters formation of the TnpB polypeptide and related complex. In one embodiment, the altered activity comprises increased cleavage activity as to off-target polynucleotide loci. Accordingly, in one embodiment, there is increased specificity for target polynucleotide loci as compared to off-target polynucleotide loci. In other embodiments, there is reduced specificity for target polynucleotide loci as compared to off-target polynucleotide loci. In one embodiment, the mutations result in decreased off-target effects (e.g. cleavage or binding properties, activity, or kinetics), such as in case for TnpB polypeptide for instance resulting in a lower tolerance for mismatches between target and the reRNA. Other mutations may lead to increased off-target effects (e.g., cleavage or binding properties, activity, or kinetics). Other mutations may lead to increased or decreased on-target effects (e.g., cleavage or binding properties, activity, or kinetics). In one embodiment, the mutations result in altered (e.g., increased or decreased) activity, association or formation of the functional nuclease complex. Examples mutations include mutation of negative or neutral residues to positively charged residues, or positively charged residues to neutral or neutral residues to negative residues and/or (evolutionary) conserved residues, such as conserved positively charged residues, in order to enhance specificity. In one embodiment, such residues may be mutated to uncharged residues, such as alanine. Because the TnpB polypeptide interacts with guide or bound DNA over the length of the TnpB polypeptide, mutation of residues across the TnpB polypeptide may be utilized for altered activity. In an aspect, the TnpB polypeptide residues for mutation are altered based on amino acid sequence positions of Deinococcus radiodurans ISDra2, see, e.g. Karvelis et al., Nature 599, 692-696 (2021).

Preferably, one or more TnpB comprises one or more mutated residues in the Rec domain and optionally these mutated residues are hydrophobic. Alternatively, one or more TnpB comprises mutated residues in the RuvC domain. Preferably, one or more of the mutated residues typically form a hydrogen bond with another TnpB monomer. More preferably, a combination of the two sets of mutations as described above.

In yet other embodiments, the TnpB-nuclease fusions are linked using a polypeptide comprising glycine and serine residues or unstructured XTEN protein polymer.

In other exemplary embodiments, the TnpB-nuclease fusions are linked using an RNA wherein the RNA comprises a guide RNA or a reRNA.

In further embodiments, the TnpB-nuclease fusions comprise one or more nuclear localization signals selected from but not limited to SV40, c-Myc, NLP-1.

Also described herein are methods and compositions for increasing the TnpB-mediated editing efficiency. In some aspects, the editing effiency is greater than 70%, at least 70.5%, at least 71%, at least 72%, at least 73%, at least 74%, at least 75%, at least 76%, at least 77%, at least 78%, at least 79%, at least 80%, at least 85%, at least 90%, at least 95%, or at least 99%.

Additionally described herein are methods and compositions for increasing the TnpB-mediated editing specificity. In some aspects, the editing specificity is greater than 70%, at least 70.5%, at least 71%, at least 72%, at least 73%, at least 74%, at least 75%, at least 76%, at least 77%, at least 78%, at least 79%, at least 80%, at least 85%, at least 90%, at least 95%, or at least 99%.

### C. TnpB Accessory Domains/Proteins

In other aspect, the TnpB-based genome perturbation systems may comprise one or more additional accessory proteins having genome modifying functions, including recombinases, invertases, nucleases, polymerases, ligases, deaminases, or reverse transcriptases. In various embodiments, the accessory proteins may be provided separately. In other embodiments, the accessory proteins may be fused to TnpB, optionally with a linker.

Liu et al. has recently developed base editing as a technology that edits target nucleotides without creating DSBs or relying on HDR. Direct modification of DNA bases by Cas-fused deaminase enzymes allows for C·G to T·A, or A·T to G·C, base pair conversions in a short target window (~5-7 bases) with very high efficiency. Komor, A. C., Kim, Y. B., Packer, M. S., Zuris, J. A. & Liu, D. R. Programmable editing of a target base in genomic DNA without double-stranded DNA cleavage. Nature 533, 420-424 (2016). Nishida, K. et al. Targeted nucleotide editing using hybrid prokaryotic and vertebrate adaptive immune systems. Science 353, aaf8729 (2016). 6. Gaudelli, N. M. et al. Programmable base editing of A·T to G·C in genomic DNA without DNA cleavage. Nature 551, 464-471 (2017). Kim, Y. B. et al. Increasing the genome-targeting scope and precision of base editing with engineered Cas9-cytidine deaminase fusions. Nat. Biotechnol. 35, 371-376 (2017). 25. Li, X. et al. Base editing with a Cpf1-cytidine deaminase fusion. Nat. Biotechnol. 36, 324-327 (2018). Gehrke, J. M. et al. An APOBEC3A-Cas9 base editor with minimized bystander and off-target activities. Nat. Biotechnol. (2018). doi: 10.1038/nbt.4199. Rees, H. A. & Liu, D. R. Base editing: precision chemistry on the genome and transcriptome of living cells. Nat. Rev. Genet. 1 (2018). doi: 10.1038/s41576-018-0059-1.

Accordingly, in various aspects of the present disclosure, the TnpB is fused to a deaminase suitable for base editing. In some embodiments, the deaminase is selected from an adenosine deaminase, E. coli tRNA adenosine, or TadA deaminase wherein TadA is engineered for higher efficiency in human cells in comparison to pWT TadA base editor. In certain embodiments, TadA is engineered through directed evolution.

In certain other embodiments, the deaminase comprises a cytidine deaminase. Preferably, the cytidine deaminase is engineered for higher efficiency in human cells in comparison to wild type cytidine deaminase base editor. In further embodiments, the TnpB genome editing system contains one or more uracil glycosylase inhibitor.

In yet other embodiments, the TnpB-deaminase fusions are linked using a polypeptide comprising glycine and serine residues or unstructured XTEN protein polymer.

In further embodiments, the TnpB RuvC domain is mutated wherein the mutation slows cleavage of the target strand or slows the cleavage of the non-target strand. In other embodiments, the TnpB is mutated to be catalytically inactive.

In certain preferred embodiments one or more deaminase is fused to a TnpB dimer. In certain embodiments, the deaminase is fused to the N-terminus of TnpB. In other embodiments, the deaminase is fused to the C-terminus of TnpB. In further embodiments, the deaminase is placed in various locations of the TnpB including without limitations: inside the Rec-domain of the TnpB, after the Rec-domain of the TnpB, in the Wedge domain of TnpB, after the Wedge domain of TnpB, in the RuvC domain of TnpB, after the RuvC domain of TnpB, in the Helical hairpin domain of TnpB, after the Helical hairpin domain of TnpB, in the ZnF domain of TnpB, after the Znf domain of TnpB. The present disclosure contemplates placement of the deaminase in and around or near or adjacent to the aforementioned domains.

In certain alternative embodiments, the TnpB fusion protein is co-expressed with one or more TnpB not fused to a deaminase. In other embodiments, the unfused TnpB is mutated to be catalytically inactive. In other examples, the TnpB fusion contains one or more nuclear localization signals selected or derived from SV40, c-Myc or NLP-1.

In other exemplary embodiments, the TnpB-deaminase fusions bind to a guide RNA or a reRNA. In instances where the TnpB system is fused to a polypeptide that modulates host-repair. In some examples, the polypeptide is a uracil glycosylase inhibitor. In other examples, the polypeptide inhibits mismatch repair wherein the MMR inhibiting polypeptide is a dominant negative MLH1.

In various other aspects, one or more TnpB is fused to a reverse transcriptase suitable for prime editing. In some embodiments, the reverse transcriptase comprises M-MLV. In certain embodiments, the M-MLV is an engineered reverse transcriptase variant designed to improve processivity, efficiency, and/or fidelity. In various embodiments, the reverse transcriptase is derived from the human genome or derived from a human endogenous retrovirus.

In one embodiment, the accessory function that is added or otherwise coupled or attached to a TnpB polypeptide (e.g., deaminase or reverse transcriptase) provides for a TnpB-based system that is capable of performing a specialized function or activity (e.g., base editing or prime editing). For example, the TnpB protein may be fused, operably coupled to, or otherwise associated with one or more heterologous functionals domains. In certain example embodiments, the TnpB protein may be a catalytically dead TnpB protein and/or have nickase activity. A nickase is an TnpB protein that cuts only one strand of a double stranded target. In such embodiments, the catalytically inactive TnpB or nickase provide a sequence specific targeting functionality via the coRNA that delivers the functional domain to or proximate a target sequence.

It is also contemplated that the TnpB complex as a whole may be associated with two or more functional domains. For example, there may be two or more functional domains associated with the TnpB polypeptide, or there may be two or more functional domains associated with the reRNA component (via one or more adaptor proteins or aptamers), or there may be one or more functional domains associated with the TnpB polypeptide and one or more functional domains associated with the reRNA component.

In one embodiment, one or more functional domains are associated with a TnpB polypeptide via an adaptor protein, for example as used with the modified guides of Konnerman et al. (Nature 517, 583-588, 29 Jan. 2015). In one embodiment, the one or more functional domains is attached to the adaptor protein so that upon binding of the TnpB polypeptide to reRNA and target, the functional domain is in a spatial orientation allowing for the functional domain to function in its attributed function.

Exemplary functional accessory domains that may be fused to, operably coupled to, or otherwise associated with an TnpB protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, a ligase domain, a topoisomerase domain, a deaminase domain, a polymerase domain (e.g., reverse transcriptase), an integrase domain, and combinations thereof. In an embodiment, the functional domain is an HNH domain, and may be used with a naturally catalytically inactive TnpB protein to engineer a nickase. Methods for generating catalytically dead TnpB or a nickase TnpB can be adapted from approaches in Cas9 proteins, see, for example, WO 2014/204725, Ran et al. Cell. 2013 Sep. 12; 154(6): 1380-1389, known in the art and incorporated herein by reference. Briefly, one or more mutations in the catalytic domain of the RuvC domain and/or the HNH domain of the TnpB protein can be introduced that may reduce or abolish NHEJ activity. In an aspect, at least one mutation in the RuvC domain and at least one mutation in the HNH domain is provided. In an embodiment, the TnpB polypeptide comprises a mutation at D191 and/or E278 based on amino acid sequence positions of Deinococcus radiodurans ISDra2. In an aspect, the amino acid mutations comprise D191A and/or E278A based on amino acid sequence positions of Deinococcus radiodurans ISDra2.

In one embodiment, the functional domains can have one or more of the following activities: nucleobase deaminase activity, reverse transcriptase activity, retrotransposase activity, transposase activity, integrase activity, recombinase activity, topoisomerase activity, ligase activity, polymerase activity, helicase activity, methylase activity, demethylase activity, translation activation activity, translation initiation activity, translation repression activity, transcription activation activity, transcription repression activity, transcription release factor activity, histone modification activity, nuclease activity (e.g. VirD2), single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In one embodiment, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) betagalactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the TnpB protein. In embodiments having two or more functional domains, each of the two can be positioned at or near or in proximity to a terminus of the TnpB protein. In one embodiment, such as those where the functional domain is operably coupled to the effector protein, the one or more functional domains can be tethered or linked via a suitable linker (including, but not limited to, GlySer linkers) to the TnpB protein. When there is more than one functional domain, the functional domains can be same or different. In one embodiment, all the functional domains are the same. In one embodiment, all of the functional domains are different from each other. In one embodiment, at least two of the functional domains are different from each other. In one embodiment, at least two of the functional domains are the same as each other.

In additional embodiments, the TnpB-deaminase fusion protein is co-expressed with a TnpB not fused to a reverse transcriptase. Preferably, the unfused TnpB is mutated to be catalytically inactive, however, fused TnpB may also be mutated to be catalytically inactive, either or both. Various TnpB-RT fusion protein binds to a truncated reRNA or to a truncated guide RNA. In some embodiments, this maintains DNA binding activity but slows cleavage kinetics or deactivates DNA cleavage partially or entirely. Additional embodiments, include the reverse transcriptase fused to the N-terminus of TnpB or to the C-terminus of TnpB. In further embodiments, the reverse transcriptase is placed inside the Rec-domain of the TnpB, after the Rec-domain of the TnpB, in the Wedge domain of TnpB, after the Wedge domain of TnpB, in the RuvC domain of TnpB, after the RuvC domain of TnpB, in the Helical hairpin domain of TnpBafter the Helical hairpin domain of TnpB, in the ZnF domain of TnpB, after the Znf domain of TnpB.

Preferably, the TnpB-RT fusion protein is bound to an engineered reRNA wherein the engineered reRNA contains a 5′ extension, the engineered reRNA contains a 3′ extension, the extensions contain a template for a desired edit, the extension contains homology to the target site, the extension contains homology to the human genome, the extension contains sequence encoding a landing-pad for a homing integrase and/or recombinase. In preferred embodiments, the TnpB-RT fusion protein is fused or cleaved. In certain embodiments, the TnpB-RT system is fused to a polypeptide that modulates host-repair, wherein the polypeptide is a uracil glycosylase inhibitor, wherein the polypeptide inhibits mismatch repair, wherein the MMR inhibiting polypeptide is a dominant negative MLH1.

In various aspects of the present disclosure, the TnpB fused to a transcriptional modulating polypeptide suitable for transcriptional interference, activation or epigenetic editing.

In some embodiments, the TnpB-transcriptional modulating polypeptide fusions comprise one or more nuclear localization signals selected or derived from SV40, c-Myc or NLP-1.

In other embodiments, the TnpB-transcriptional modulating polypeptide fusion proteins bind to a truncated guide RNA. In further embodiments, the TnpB-transcriptional modulating polypeptide comprises glycine and serine residues. In yet other embodiments, the TnpB-transcriptional modulating polypeptide are linked to one or more unstructured XTEN protein polymers.

In various embodiments, the transcriptional modulating polypeptide of the TnpB-transcriptional modulating polypeptide fusion performs histone acetylation or comprises histone acetyltransferase (HAT) p300 activity.

In other embodiments, the transcriptional modulating polypeptide of the TnpB-transcriptional modulating polypeptide fusion performs histone demethylation or comprises lysine-specific demethylase (LSD1) activity.

In further embodiments, the transcriptional modulating polypeptide of the TnpB-transcriptional modulating polypeptide fusion performs cystine methylation or comprises one or more activities selected from DNA (cytosine-5)-methyltransferase (DNMT3A), DNA-methyltransferase 3-like (DNMT3L) and MQ1.

In other embodiments, the transcriptional modulating polypeptide of the TnpB-transcriptional modulating polypeptide fusion performs cystine demethylation or comprises TET1 activity.

In additional embodiments, the transcriptional modulating peptide of the TnpB-transcriptional modulating polypeptide fusion is a transcriptional repressor or comprises a KRAB domain. Alternatively, the transcriptional modulating peptide of the TnpB-transcriptional modulating polypeptide fusion is a transcriptional activator or comprises one or more activators including without limitation, for example, HS1, VP64 and p65.

In other embodiments, Where the the transcriptional modulating peptide of the TnpB-transcriptional modulating polypeptide fusion is a repressor or comprises multiple transcriptional modulating peptides. In yet other embodiments, the TnpB of the TnpB-transcriptional modulating polypeptide fusion is mutated to be catalytically inactive.

In further embodiments, the transcriptional modulating peptides of the TnpB-transcriptional modulating polypeptide fusion are physically coupled through an engineered reRNA wherein the reRNA comprises one or more aptamers.

In additional embodiments, the transcriptional modulating peptides of the TnpB-transcriptional modulating polypeptide fusion are physically coupled through an engineered guide RNA, wherein the guide RNA contains one or more aptamers.

D. reRNA

The TnpB systems herein may further comprise one or more nucleic acid components, which are also referred to herein as reRNA. As reported in Karvelis et al., “Transposon-associated TnpB is a programmable RNA-guided DNA endonuclease,” Nature, Nov. 25, 2021, Vol. 599, pp. 692-700 (incorporated herein by reference), TnpB is an RNA-guided dsDNA nuclease that forms a complex with a non-coding RNA called “reRNA.” The reRNA is a transcript that is generated from the transcription of the IS DNA sequence beginning at a transcription initiation site located within the 3′ end of the TnpB coding region and ending at a transcription termination site located in the flanking genomic DNA region that is immediately downstream of the RE of the Insertion Sequence. Thus, the reRNA comprises three regions: (a) a region corresponding to the 3′ end of the TnpB coding region, (b) a region corresponding to the RE, and (c) a region corresponding to the flanking genomic DNA immediately downstream of the 3′ end of the RE. Regions (a) and (b) generally form a folded scaffold that appears to bind to the TnpB protein. Region (c) functions as a spacer or targeting sequence which allows for the targeting of a TnpB-reRNA complex to a target site to which the region (c) has complementarity to and anneals. Region (c), in various embodiments, can be engineered to be any desired target sequence such that the TnpB-reRNA complex is targeted to a desired target sequence.

Thus, the reRNA sequence may be predicted from the sequence of the region spanning the 3′ end of the TnpB coding region through a flanking region downstream of the RE.

Computational methods can be used to predict the reRNA sequences for identified TnpB and TnpB-like proteins. As reported in Karvelis et al., “Transposon-associated TnpB is a programmable RNA-guided DNA endonuclease,” Nature, Nov. 25, 2021, Vol. 599, pp. 692-700, the TnpB protein co-purified with an RNA molecule of about 150 nucleotides long which had a sequence that was derived from the IS and a sequence downstream of the IS.

In various embodiments, reRNA may be engineered to include RNA, DNA, or combinations of both and include modified and non-canonical nucleotides as described further below. The reRNA can comprise a reprogrammable spacer sequence and a scaffold that interacts with the TnpB polypeptide. reRNA may form a complex with a TnpB polypeptide, and direct sequence-specific binding of the complex to a target sequence of a target polynucleotide. In one example embodiment, the reRNA is a single molecule comprising a scaffold sequence and a spacer sequence. In certain example embodiments, the spacer is 5′ of the scaffold sequence. In one example embodiment, the reRNA may further comprise a conserved nucleic acid sequence between the scaffold and spacer portions.

In embodiments, the reRNA comprises a spacer sequence and a scaffold sequence, e.g. a conserved nucleotide sequence. In embodiments, the reRNA comprises about 45 to about 350 nucleotides, or about 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 17, 138, 19, 140, 141, 142, 143, 144, 145, 146, 147, 148, 149, 150, 151, 152, 153, 154, 155, 156, 157, 158, 159, 160, 11, 162, 163, 164, 165, 166, 167, 168, 169, 170, 171, 172, 173, 174, 175, 176, 177, 178, 179, 180. 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 2340, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 272, 273, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 333, 334, 335, 336, 337, 338, 339, 340, 341, 342, 343, 344, 345, 346, 347, 348, 349, or 350 nucleotides.

In embodiments, the reRNA comprises a scaffold sequence, e.g. a conserved nucleotide sequence that binds to the TnpB protein. The scaffold sequence therefore typically comprises conserved regions, with the scaffold comprising about 30 to 200 nucleotides, about 50 to 180, about 80 to 175 nucleotides, or about 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or more nucleotides.

The reRNA may further comprise a spacer, which can be re-programmed to direct site specific binding to a target sequence of a target polynucleotide. The spacer may also be referred to herein as part of the reRNA scaffold or reRNA, and may comprise an engineered heterologous sequence.

In one embodiment, the spacer length or targeting sequence length of the reRNA is from 10 to 50 nt. In one embodiment, the spacer length of the oRNA is at least 10, 11, 12, 13, 14, or 15 nucleotides. In one embodiment, the spacer length is from 10 to 40 nucleotides, from 15 to 30 nt, 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In example embodiments, the spacer sequence is 14, 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, or 50 nt.

As used herein, the term “spacer” may also be referred to as a “guide sequence” or “targeting sequence” which has complementarity to a target sequence (e.g., a desired target gene in a genome which is desired to be edited). In one embodiment, the degree of complementarity of the spacer sequence to a given target sequence, when optimally aligned using a suitable alignment algorithm, is about or more than 50%, 60%, 75%, 80%, 85%, 90%, 95%, 97.5%, 99%, or more. In certain example embodiments, the reRNA molecule comprises a spacer sequence that may be designed to have at least one mismatch with the target sequence, such that a RNA duplex formed between the sequence and the target sequence. Accordingly, the degree of complementarity is less than 99%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies), ELAND (Illumina, San Diego, CA), SOAP (available at soap.genomics.org.cn), and Maq (available at maq.sourceforge.net).

The ability of a sequence (within a nucleic acid-targeting reRNA molecule) to direct sequence-specific binding of a nucleic acid-targeting complex to a target nucleic acid sequence may be assessed by any suitable assay. For example, the components of a reRNA system sufficient to form a TnpB-targeting complex, including the reRNA molecule sequence to be tested, may be provided to a host cell having the corresponding target nucleic acid sequence, such as by transfection with vectors encoding the components of the TnpB-targeting complex, followed by an assessment of preferential targeting (e.g., cleavage) within the target nucleic acid sequence. Similarly, cleavage of a target nucleic acid sequence (or a sequence in the vicinity thereof) may be evaluated in a test tube by providing the target nucleic acid sequence, components of a TnpB-targeting complex, including the sequence to be tested and a control sequence different from the test coRNA, and comparing binding or rate of cleavage at or in the vicinity of the target sequence between the test and control reRNA molecule sequence reactions. Other assays are possible, and will occur to those skilled in the art. A spacer sequence, and hence a nucleic acid targeting reRNA may be selected to target any target nucleic acid sequence.

E. reRNA Modifications

In one embodiment, the reRNA comprises non-naturally occurring nucleic acids and/or non-naturally occurring nucleotides and/or nucleotide analogs, and/or chemical modifications. Preferably, these non-naturally occurring nucleic acids and non-naturally occurring nucleotides are located outside the reRNA sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the present disclosure, a reRNA component nucleic acid comprises ribonucleotides and non-ribonucleotides. In one such embodiment, a reRNA component comprises one or more ribonucleotides and one or more deoxyribonucleotides. In an embodiment of the present disclosure, the reRNA component comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA).

Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of coRNA chemical modifications include, without limitation, incorporation of 2′-O-methyl (M), 2′-O-methyl 3′phosphorothioate (MS), S-constrained ethyl (cEt), or 2′-O-methyl 3′ thioPACE (MSP) at one or more terminal nucleotides. Such chemically modified oRNA components can comprise increased stability and increased activity as compared to unmodified oRNA components, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 D01: 10.1038/s41551-017-0066). In one embodiment, the 5′ and/or 3′ end of a reRNA component is modified by a variety of functional moieties including fluorescent dyes, polyethylene glycol, cholesterol, proteins, or detection tags. (See Kelly et al., 2016, J. Biotech. 233:74-83). In one embodiment, a reRNA component comprises ribonucleotides in a region that binds to a target sequence and one or more deoxyribonucletides and/or nucleotide analogs in a region that binds to the TnpB polypeptide.

In an embodiment, deoxyribonucleotides and/or nucleotide analogs are incorporated in engineered reRNA component structures. In one embodiment, 3-5 nucleotides at either the 3′ or the 5′ end of a reRNA component is chemically modified. In one embodiment, only minor modifications are introduced in the seed region, such as 2′-F modifications. In one embodiment, 2′-F modification is introduced at the 3′ end of a reRNA component. In one embodiment, three to five nucleotides at the 5′ and/or the 3′ end of the reRNA component are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33(9): 985-989). In one embodiment, all of the phosphodiester bonds of a reRNA component are substituted with phosphorothioates (PS) for enhancing levels of gene disruption. In one embodiment, more than five nucleotides at the 5′ and/or the 3′ end of the reRNA component are chemically modified with 2′-O-Me, 2′-F or S-constrained ethyl (cEt). Such chemically modified reRNA component can mediate enhanced levels of gene disruption (see Ragdarm et al., 0215, PNAS, E7110-E7111). In an embodiment of the present disclosure, a reRNA component is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the reRNA component by a linker, such as an alkyl chain. In one embodiment, the chemical moiety of the modified nucleic acid component can be used to attach the reRNA component to another molecule, such as DNA, RNA, protein, or nanoparticles. Such chemically modified reRNA component can be used to identify or enrich cells generically edited by a TnpB polypeptide and related systems (see Lee et al., eLife, 2017, 6:e25312, DOI: 10.7554).

Other reRNA modifications are described in Kim, D. Y., Lee, J. M., Moon, S. B. et al. Efficient CRISPR editing with a hypercompact Cas12f1 and engineered guide RNAs delivered by adeno-associated virus. Nat Biotechnol 40, 94-102 (2022).

Accordingly, in various aspects of the present disclosure, the reRNA are modified in one or more TnpB reRNA. MS1, an internal penta (uridinylate) (UUUUU) sequence in the tracrRNA; MS2, the 3′ terminus of the crRNA; MS3, the ‘stem 1’ region of the tracrRNA; MS4, the tracrRNA-crRNA complementary region; and MS5, the ‘stem 2’ region of the tracrRNA.

Various aspects of the present disclosure provide methods and compositions for improved reRNA stability via chemical modifications. Braasch, D. A., Jensen, S., Liu, Y., Kaur, K., Arar, K., White, M. A., et al. (2003). RNA interference in mammalian cells by chemically-modified RNA. Biochemistry 42, 7967-7975. Chiu, Y. L., and Rana, T. M. (2003). siRNA function in RNAi: a chemical modification analysis. RNA 9, 1034-1048. Behlke, M. A. (2008). Chemical modification of siRNAs for in vivo use. Oligonucleotides 18, 305-319. Bennett, C. F., and Swayze, E. E. (2010). RNA targeting therapeutics: molecular mechanisms of antisense oligonucleotides as a therapeutic platform. Annu. Rev. Pharmacol. Toxicol. 50, 259-293. Deleavey, G. F., and Damha, M. J. (2012). Designing chemically modified oligonucleotides for targeted gene silencing. Chem. Biol. 19, 937-954. Lennox, K. A., and Behlke, M. A. (2020). Chemical modifications in RNA interference and CRISPR/Cas genome editing reagents. Methods Mol. Biol. 2115, 23-55.

For instance, Hendel et al. improved guideRNA stability by chemically modifying gRNA ends to reduce degradation by exonucleases, RNA nuclease. Hendel, A., Bak, R. O., Clark, J. T., Kennedy, A. B., Ryan, D. E., Roy, S., et al. (2015a). Chemically modified guide RNAs enhance CRISPR-Cas genome editing in human primary cells. Nat. Biotechnol. 33, 985-989. Chemical modifications of gRNAs may enable more efficient and safer gene-editing in primary cells suitable for clinical applications.

A review of types of chemical modifications are provided in the table below. Allen, Daniel et al. “Using Synthetically Engineered Guide RNAs to Enhance CRISPR Genome Editing Systems in Mammalian Cells.” Frontiers in genome editing vol. 2 617910. 28 Jan. 2021.

| | | Effect on |
|---|---|---|
| | | genome editing |
| Modification(s) | Modification location | efficiency |
| M | Terminal residues | ↑# |
| MS | Terminal residues | ↑# |
| | Spacer (PAM-distal region) | ↑* |
| | Spacer (tracrRNA-binding region) | ↑* |
| | Spacer (Seed region) | ↓ |
| MSP | Terminal residues | ↑# |
| cEt | Spacer (PAM-distal region) | ↑ |
| | Spacer (tracrRNA-binding) | ↑ |
| | Spacer (Seed region) | ↓ |
| 2′-F | Spacer (PAM-distal region) | ↑ |
| | Spacer (tracrRNA-binding) | ↑ |
| | Spacer (Seed region) | ↓ |
| 2′-F + PS | Spacer (PAM-distal region) | ↑ |
| | Spacer (tracrRNA-binding) | ↑ |
| | Spacer (Seed region) | ↓ |
| | Spacer (Seed region, Cas9- | ↑* |
| | non-interacting residues) | |
| PS | Whole crRNA | ↑ |

*additionally validated in vivo;
#additionally validated in human primary cells
2′-O-methyl (M or 2′-O—Me);
2′-O-methyl 3′phosphorothioate (MS);
2′-O-methyl-3′-thioPACE (MSP);
S-constrained ethyl (cET);
2′-fluoro (2′-F);
phosphorothioate (PS)

Accordingly, in various embodiments of the present disclosure, the genome editing system comprising TnpB and further comprises one or more chemical modifications selected from, but not limited to the modifications in the above table.

In exemplary embodiments, chemical modifications to the reRNA include modifications on the ribose rings and phosphate backbone of reRNAs and modifications at the 2′OH include 2′-O-Me, 2′-F, and 2′F-ANA. More extensive ribose modifications include 2′F-4′-Cα—OMe and 2′,4′-di-Cα-OMc combine modification at both the 2′ and 4′ carbons. Phosphodiester modifications include sulfide-based Phosphorothioate (PS) or acetate-based phosphonoacetate alterations. Combinations of the ribose and phosphodiester modifications have given way to formulations such as 2′-O-methyl 3′phosphorothioate (MS), or 2′-O-methyl-3′-thioPACE (MSP), and 2′-O-methyl-3′-phosphonoacetate (MP) RNAs. Locked and unlocked nucleotides such as locked nucleic acid (LNA), bridged nucleic acids (BNA), S-constrained ethyl (cEt), and unlocked nucleic acid (UNA) are examples of sterically hindered nucleotide modifications. Modifications to make a phosphodiester bond between the 2′ and 5′ carbons (2′,5′-RNA) of adjacent RNAs as well as a butane 4-carbon chain link between adjacent RNAs have been described.

Any of the above TnpB editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

### Integrase Editors (e.g., PASTE)

In some embodiments, the gene editing system comprises one or more integrase editors. In certain embodiments, the gene editing system comprises a construct enabling programmable addition via site-specific targeting elements (PASTE). In certain embodiments, the gene editing system comprises one or more integrase editors and/or gene editing systems described and disclosed in PCT Publications WO2022087235A1, WO2020191245A1, WO2022060749A1, WO2021188840A1, WO2021138469A1, US Patent Application Publications US20140349400A1, US20210222164A1 or US20150071898A1, each of which is incorporated by reference herein in their entirety. In certain embodiments, the one or more integrase editors comprise CRISPR directed integrases disclosed in Yarnall, M. T. N., Ioannidi, E. I., Schmitt-Ulms, C. et al. Drag-and-drop genome insertion of large sequences without double-strand DNA cleavage using CRISPR-directed integrases. Nat Biotechnol (2022).

### Epigenetic Editors

In still other embodiments, the LNPs may be used to deliver an epigenetic editing system. Epigenetic editors are generally composed of an epigenetic enzyme or their catalytic domain fused with a user-programmable DNA-binding protein, such as a CRISPR-Cas enzyme or TnpB enzyme. The user-programmable DNA-binding protein (plus a guide RNA in the case of a nucleic acid programmable DNA binding protein) guides the epigenetic enzyme (e.g., a DNA methyltransferase or DNMT) to a specific site (e.g., a CpG island in a promoter region of a gene) in order to induce a change in promoter activity.

Epigenetic modifications of DNA and histones are known for their multifaceted contributions to transcriptional regulation. As these modifications are faithfully propagated throughout DNA replication, they are considered central players in cellular memory of transcriptional states. Many efforts in the last decade have generated a vast understanding of individual epigenetic modifications and their contribution to transcriptional regulation. Epigenetic editing offers powerful tools to selectively induce epigenetic changes in a genome without altering the sequence of a nucleotide sequence as a means to regulate gene activity. The foundation of epigenetic editing is formed by the ability to generate fusion proteins of epigenetic enzymes or their catalytic domains with programmable DNA-binding platforms such as the clustered regularly interspaced short palindromic repeat (e.g., CRISPR Cas9 or Cas12a) to target these to an endogenous locus of choice. The enzymatic fusion protein then dictates the initial deposited modification while subsequent cross-talk within the local chromatin environment likely influences epigenetic and transcriptional output.

The following published literature discussing epigenetic editing is incorporated herein by reference each in their entireties.
Gjaltema R A F, Rots M G. Advances of epigenetic editing. Curr Opin Chem Biol. 2020 August; 57:75-81. Epub 2020 Jun. 30. PMID: 32619853.
Kleinstiver B P, Sousa A A, Walton R T, Tak Y E, Hsu J Y, Clement K, Welch M M, Horng J E, Malagon-Lopez J, Scarfó I, Maus M V, Pinello L, Aryee M J, Joung J K. Engineered CRISPR-Cas12a variants with increased activities and improved targeting ranges for gene, epigenetic and base editing. Nat Biotechnol. 2019 March; 37(3):276-282. Epub 2019 Feb. 11. Erratum in: Nat Biotechnol. 2020 July; 38(7):901. PMID: 30742127; PMCID: PMC6401248.
Rots M G, Jeltsch A. Editing the Epigenome: Overview, Open Questions, and Directions of Future Development. Methods Mol Biol. 2018; 1767:3-18. PMID: 29524127.
Liu X S, Jaenisch R. Editing the Epigenome to Tackle Brain Disorders. Trends Neurosci. 2019 December; 42(12):861-870. Epub 2019 Nov. 7. PMID: 31706628.
Waryah C B, Moses C, Arooj M, Blancafort P. Zinc Fingers, TALEs, and CRISPR Systems: A Comparison of Tools for Epigenome Editing. Methods Mol Biol. 2018; 1767:19-63. PMID: 29524128.
Xu X, Hulshoff M S, Tan X, Zeisberg M, Zeisberg E M. CRISPR/Cas Derivatives as Novel Gene Modulating Tools: Possibilities and In Vivo Applications. Int J Mol Sci. 2020 Apr. 25; 21(9):3038. PMID: 32344896; PMCID: PMC7246536.

In addition, the following published patent literature relating to epigenetic editing is incorporated herein by reference each in their entireties.

| Pub Number | Title |
|---|---|
| WO2023283359A2 | COMPOSITIONS AND METHODS FOR MODULATING SECRETED |
| | FRIZZLED RECEPTOR PROTEIN 1 (SFRP1) GENE EXPRESSION |
| WO2022226139A1 | TISSUE-SPECIFIC NUCLEIC ACID DELIVERY BY MIXED CATIONIC |
| | LIPID PARTICLES |
| WO2022132926A1 | TISSUE-SPECIFIC NUCLEIC ACID DELIVERY BY 1,2-DIOLEOYL-3- |
| | TRIMETHYLAMMONIUM-PROPANE (DOTAP) LIPID NANOPARTICLES |
| WO2021183720A1 | COMPOSITIONS AND METHODS FOR MODULATING FORKHEAD BOX |
| | P3 (FOXP3) GENE EXPRESSION |
| WO2021061815A1 | COMPOSITIONS AND METHODS FOR MODULATING HEPATOCYTE |
| | NUCLEAR FACTOR 4-ALPHA (HNF4α) GENE EXPRESSION |
| WO2021061707A1 | COMPOSITIONS AND METHODS FOR MODULATING APOLIPOPROTEIN |
| | B (APOB) GENE EXPRESSION |
| WO2021061698A1 | METHODS AND COMPOSITIONS FOR MODULATING FRATAXIN |
| | EXPRESSION AND TREATING FRIEDRICH'S ATAXIA |

### Gene Writing

In some embodiments, the gene editing system is a gene writing system. In certain embodiments, the gene editing system is one described and disclosed in US Patent Application Publications US2022039681A1 or US20200109398A1, each of which is incorporated by reference herein in their entirety.

In certain embodiments, the gene editing system is a system for modifying DNA comprising a polypeptide or a nucleic acid encoding a polypeptide capable of target primed reverse transcription, wherein the polypeptide comprises (a) a reverse transcriptase domain and (b) an endonuclease domain, wherein at least one of (a) or (b) is heterologous; and a template RNA comprising (i) a sequence that binds the polypeptide and (ii) a heterologous object sequence. In certain embodiments, the gene editing system is a system for modifying DNA comprising a polypeptide or a nucleic acid encoding a polypeptide capable of target primed reverse transcription, wherein the polypeptide comprises (a) a target DNA binding domain, (b) a reverse transcriptase domain and (c) an endonuclease domain, wherein at least one of (a), (b) or (c) is heterologous, and a template RNA comprising (i) a sequence that binds the polypeptide and (ii) a heterologous object sequence. In certain embodiments, the polypeptide comprises a sequence of at least 50 amino acids having at least 80% identity to a reverse transcriptase domain of a sequence of a polypeptide listed in TABLE 1, TABLE 2, or TABLE 3 of US Patent Application Publication US20200109398A1, which is incorporated by reference in its entirety, including the aforementioned sequence tables.

In certain embodiments, the reverse transcriptase domain is from a retrovirus or a retrotransposon, such as a LTR-retrotransposon, or a non-LTR retrotransposon. In certain embodiments, the reverse transcriptase is from a non-LTR retrotransposon, wherein the non-LTR retrotransposon is a RLE-type non-LTR retrotransposon from the R2, NeSL, HERO, R4, or CRE clade, or an APE-type non-LTR retrotransposon from the R1, or Tx1 clade. In certain embodiments, the reverse transcriptase domain is from an avian retrotransposase of column 8 of Table 3 of US20200109398A1, or a sequence having at least 70%, identity thereto. In certain embodiments, the reverse transcriptase domain does not comprise an RNA binding domain and the polypeptide comprises an RNA binding domain heterologous to the reverse transcriptase domain, wherein the RNA binding domain is a B-box protein, a MS2 coat protein, a dCas protein, or a UTR binding protein, or a fragment or variant of any of the foregoing.

In certain embodiments, the endonuclease domain is heterologous to the reverse transcriptase domain, and wherein the endonuclease is a Fok1 nuclease (or a functional fragment thereof), a type-II restriction 1-like endonuclease (RLE-type nuclease), another RLE-type endonuclease, or a Prp8 nuclease. In certain embodiments, the endonuclease domain is heterologous to the reverse transcriptase domain, wherein endonuclease domain contains DNA binding functionality. In certain embodiments, the endonuclease domain is heterologous to the reverse transcriptase domain, and wherein the endonuclease has nickase activity and does not form double stranded breaks.

In certain embodiments, the polypeptide comprises a DNA binding domain heterologous to the reverse transcriptase domain, and wherein the DNA binding domain is: a zinc-finger element, or a functional fragment thereof; or a TAL effector element, or a functional fragment thereof; a Myb domain; or a sequence-guided DNA binding element. In certain embodiments, the polypeptide comprises a DNA binding domain heterologous to the reverse transcriptase domain, and wherein the DNA binding element is a sequence-guided DNA binding element, further wherein the sequence-guided DNA binding element is Cas9, Cpf1, or other CRISPR-related protein. In certain embodiments, the polypeptide comprises a DNA binding domain heterologous to the reverse transcriptase domain, and wherein the DNA binding domain is a transcription factor.

In certain embodiments, the sequence-guided DNA binding element has been altered to have no endonuclease activity. In certain embodiments, the sequence-guided DNA binding element replaces the endonuclease element of the polypeptide. In certain embodiments, the editing system is capable of modifying DNA using reverse transcriptase activity, optionally in the absence of homologous recombination activity.

In certain embodiments, the gene editing system is a system for modifying DNA comprising:
a) a recombinase polypeptide selected from Rec27 (WP_021170377.1, SEQ ID NO: 1241 of US20220396813A1), Rec35 (WP_134161939.1, SEQ ID NO: 1249 of US20220396813A1), or comprising an amino acid sequence of Table 1 or 2 of US20220396813A1, or an amino acid sequence having at least 70%, 75%, 80%, 85%, 90%, 95%, 96%, 97%, 98%, or 99% identity thereto, or a nucleic acid encoding the recombinase polypeptide; and
b) a double-stranded insert DNA comprising:
(i) a DNA recognition sequence that binds to the recombinase polypeptide of (a), said DNA recognition sequence having a first parapalindromic sequence and a second parapalindromic sequence, wherein each parapalindromic sequence is about 10-30, 12-27, or 10-15 nucleotides, e.g., about 13 nucleotides, and the first and second parapalindromic sequences together comprise the parapalindromic region of a nucleotide sequence of Table 1 of US20220396813A1, or a nucleotide sequence having at least 70%, 75%, 80%, 85%, 90%, 95%, 96%, 97%, 98%, or 99% identity thereto, or having no more than 1, 2, 3, 4, 5, 6, 7, 8 sequence alterations (e.g., substitutions, insertions, or deletions) relative thereto, and said DNA recognition sequence further comprises a core sequence of about 5-10 nucleotides, e.g., about 8 nucleotides, wherein the core sequence is situated between the first and second parapalindromic sequences, and
(ii) a heterologous object sequence.

### Gene Inactivating Systems

In some embodiments, the gene editing system comprises a polypeptide or an RNA encoding a polypeptide capable of inducing a double-stranded or single-stranded break in a desired gene, thereby inactivating said gene. In certain embodiments, the gene editing system is one described and disclosed in PCT Publications WO2020028327A1, WO2020069296A1 or WO2020118041A1, each of which is incorporated by reference herein in their entirety. In certain embodiments, the gene editing system is one described and disclosed in a patent application publication disclosed below, each of which is incorporated by reference herein in their entirety:

| Pub Number | Title |
|---|---|
| WO2022271780A1 | METHODS FOR IN VIVO EDITING OF A LIVER GENE |
| WO2022261292A1 | MODIFIED GUIDE RNAS COMPRISING AN INTERNAL LINKER FOR |
| | GENE EDITING |
| WO2022221697A1 | LIPID NANOPARTICLE COMPOSITIONS |
| WO2022221696A1 | INHIBITORS OF DNA-DEPENDENT PROTEIN KINASE AND |
| | COMPOSITIONS AND USES THEREOF |
| WO2022221695A1 | LIPID NANOPARTICLE COMPOSITIONS |
| WO2022170194A2 | LYMPHOCYTE ACTIVATION GENE 3 (LAG3) COMPOSITIONS AND |
| | METHODS FOR IMMUNOTHERAPY |
| WO2022170193A2 | T-CELL IMMUNOGLOBULIN AND MUCIN DOMAIN 3 (TIM3) |
| | COMPOSITIONS AND METHODS FOR IMMUNOTHERAPY |
| WO2022170172A1 | NATURAL KILLER CELL RECEPTOR 2B4 COMPOSITIONS AND |
| | METHODS FOR IMMUNOTHERAPY |
| WO2022147133A1 | ENGINEERED T CELLS |
| WO2022140587A1 | COMPOSITIONS AND METHODS FOR GENETICALLY MODIFYING |
| | CIITA IN A CELL |
| WO2022140586A2 | COMPOSITIONS AND METHODS FOR REDUCING IILA-A IN A CELL |
| WO2022125982A1 | COMPOSITIONS AND METHODS FOR REDUCING MHC CLASS II IN |
| | A CELL |
| WO2022125968A1 | POLYNUCLEOTIDES, COMPOSITIONS, AND METHODS FOR |
| | GENOME EDITING INVOLVING DEAMINATION |
| WO2021222287A2 | METHODS OF IN VITRO CELL DELIVERY |
| WO2021220132A1 | METHODS AND COMPOSITIONS FOR OCULAR CELL THERAPY |
| WO2021188840A1 | METHODS AND COMPOSITIONS FOR DIRECTED GENOME EDITING |
| WO2021158858A1 | COMPOSITIONS AND METHODS FOR KALLIKREIN (KLKB1) GENE |
| | EDITING |
| WO2021119275A1 | MODIFIED GUIDE RNAS FOR GENE EDITING |
| WO2020219876A1 | IONIZABLE AMINE LIPIDS AND LIPID NANOPARTICLES |
| WO2020198706A1 | COMPOSITIONS AND METHODS FOR TTR GENE EDITING AND |
| | TREATING ATTR AMYLOIDOSIS COMPRISING A |
| | CORTICOSTEROID OR USE THEREOF |
| WO2020198697A1 | COMPOSITIONS AND METHODS COMPRISING A TTR GUIDE RNA |
| | AND A POLYNUCLEOTIDE ENCODING AN RNA-GUIDED DNA |
| | BINDING AGENT |
| WO2020198641A2 | POLYNUCLEOTIDES, COMPOSITIONS, AND METHODS FOR |
| | POLYPEPTIDE EXPRESSION |
| WO2020118041A1 | MODIFIED AMINE LIPIDS |
| WO2020084580A1 | METHODS AND COMPOSITIONS FOR OCULAR CELL THERAPY |
| WO2020082047A1 | COMPOSITIONS AND METHODS FOR TREATING ALPHA-1 |
| | ANTITRYPSIN DEFICIENCEY |
| WO2020082046A2 | COMPOSITIONS AND METHODS FOR EXPRESSING FACTOR IX |
| WO2020082042A2 | COMPOSITIONS AND METHODS FOR TRANSGENE EXPRESSION |
| | FROM AN ALBUMIN LOCUS |
| WO2020082041A1 | NUCLEIC ACID CONSTRUCTS AND METHODS OF USE |
| WO2020081613A1 | COMPOSITIONS AND METHODS FOR IMMUNOTHERAPY |
| WO2020072605A1 | IONIZABLE AMINE LIPIDS |
| WO2020069296A1 | COMPOSITIONS AND METHODS FOR LACTATE DEHYDROGENASE |
| | (LDHA) GENE EDITING |
| WO2020028327A1 | COMPOSITIONS AND METHODS FOR HYDROXYACID OXIDASE 1 |
| | (HAO1) GENE EDITING FOR TREATING PRIMARY |
| | HYPEROXALURIA TYPE 1 (PH1) |
| WO2019237069A1 | MODIFIED GUIDE RNAS FOR GENE EDITING |
| WO2019237035A1 | COMPOSITIONS AND METHODS FOR IMMUNOONCOLOGY |
| WO2019067999A1 | IN VITRO METHOD OF MRNA DELIVERY USING LIPID |
| | NANOPARTICLES |
| WO2019067992A1 | FORMULATIONS |
| WO2019067910A1 | POLYNUCLEOTIDES, COMPOSITIONS, AND METHODS FOR |
| | GENOME EDITING |
| WO2019067872A1 | COMPOSITIONS AND METHODS FOR TTR GENE EDITING AND |
| | TREATING ATTR AMYLOIDOSIS |
| WO2019036513A1 | STABILIZED NUCLEIC ACIDS ENCODING MESSENGER |
| | RIBONUCLEIC ACID (MRNA) |
| WO2019003193A1 | METHODS FOR THE TREATMENT OF DISEASE WITH GENE |
| | EDITING SYSTEMS |
| WO2018175636A2 | COMPOSITIONS AND METHODS FOR IMMUNOONCOLOGY |
| WO2018142364A1 | COMPOSITIONS AND METHODS FOR THE TREATMENT OF |
| | HEMOGLOBINOPATHIES |
| WO2018119182A1 | COMPOSITIONS AND METHODS FOR TREATING ALPHA-1 |
| | ANTITRYPSIN DEFICIENCY |
| WO2018107028A1 | MODIFIED GUIDE RNAS |
| WO2018053070A1 | IMPROVED METHODS FOR ANALYZING EDITED DNA |
| WO2017185054A1 | COMPOSITIONS AND METHODS FOR TREATMENT OF DISEASES |
| | ASSOCIATED WITH TRINUCLEOTIDE REPEATS IN |
| | TRANSCRIPTION FACTOR FOUR |
| WO2017173054A1 | LIPID NANOPARTICLE FORMULATIONS FOR CRISPR/CAS |
| | COMPONENTS |
| WO2017160752A1 | METHODS AND COMPOSITIONS FOR GENE EDITING |
| WO2017115268A1 | COMPOSITIONS AND METHODS FOR THE TREATMENT OF |
| | HEMOGLOBINOPATHIES |
| WO2017093969A1 | COMPOSITIONS AND METHODS FOR IMMUNOONCOLOGY |

Compositions that Increase Gene Editing Efficiency

In some embodiments, the gene editing system comprises a polypeptide, or a nucleic acid that encodes a polypeptide, that increases gene editing efficiency. In some embodiments, the gene editing system comprises a composition described and disclosed in US Application Publication US20220090064A1, which is incorporated by reference herein in its entirety. In some embodiments, the composition comprises a guide nucleic acid, a Cas9 nickase, and/or a reverse transcriptase. The reverse transcriptase may be fused to the Cas9 nickase. The reverse transcriptase may heterodimerize with the Cas9 nickase. The reverse transcriptase may bind to a guide nucleic acid. The reverse transcriptase may be engineered to increase processivity. The guide nucleic acid may be engineered to facilitate synthesis or editing of a sequence. The guide nucleic acid may comprise a region that binds to another region on the guide nucleic acid to improve gene editing.

In some embodiments, the composition comprises a Cas 9 nickase and a reverse transcriptase, or one or two polynucleotides encoding the Cas 9 nickase and reverse transcriptase, wherein:
(i) the composition comprises a first polypeptide chain comprising the Cas nickase or a segment of the Cas nickase, and a second polypeptide chain comprising the reverse transcriptase, or the one or two polynucleotides encoding the polypeptide chains, wherein the polypeptide chains comprise leucine zippers that bind one another, or
(ii) the composition comprises a first polypeptide chain comprising a first segment of the Cas nickase, and a second polypeptide chain comprising a second segment of the Cas nickase and the reverse transcriptase, or the one or two polynucleotides encoding the polypeptide chains, wherein the polypeptide chains comprise inteins that bind one another, the Cas nickase comprises an amino acid sequence at least 80% identical to SEQ ID NO: 32, the first and second polypeptide chains respectively comprise amino acids 1-1124 and 1125-1368 of the Cas nickase, 1-1129 and 1130-1368 of the Cas nickase, 1-1139 and 1140-1368 of the Cas nickase, 1-1167 and 1168-1368 of the Cas nickase, 1-1172 and 1173-1368 of the Cas nickase, or 1-1202 and 1203-1368 of the Cas nickase, and the Cas nickase comprises a mutation at amino acid position 1030 or after amino acid position 1030 with regard to SEQ ID NO: 32, the mutation comprising a point mutation to a cysteine, threonine, alanine, or serine, or an insertion of a cysteine, threonine, alanine, or serine at the C-terminal half of the Cas9 nickase or
(iii) the reverse transcriptase comprises a Moloney leukemia virus reverse transcriptase (mlvRT) comprising an amino acid sequence at least 80% identical to SEQ ID NO: 13 or at least 80% identical to a functional fragment thereof comprising at least 400 amino acids, and a point mutation at amino acid position Q84, L139, Q221, V223, T664, or L671 with regard to SEQ ID NO: 13; wherein the respective SEQ ID NOs are those disclosed in US Application Publication US20220090064A1.

In certain embodiments, the composition comprises a guide nucleic acid comprising:
optionally, a spacer reverse complementary to a first region of a target nucleic acid, wherein the spacer is included in the guide nucleic acid, or the spacer is included in a second, different guide nucleic acid when not included in the guide nucleic acid; a scaffold configured to bind to a Cas nuclease; a reverse transcriptase template encoding a sequence to be reverse transcribed into a first synthesized strand to be inserted into the target nucleic acid; a first strand primer binding site reverse complementary to a second region of the target nucleic acid; and at least one of:
(i) a guide nucleic acid positioning system (GPS) region and a GPS binding site that hybridizes to the GPS region, wherein the GPS region and the GPS binding site are at least 10 nucleotides in length and are at least 60% reverse complementary to each other, and wherein hybridization of the GPS region and the GPS binding site positions the first strand primer binding site closer to the second region of the target nucleic acid,
(ii) a GPS region that hybridizes to a GPS binding site on the second guide nucleic acid, wherein the GPS region and the GPS binding site are at least 10 nucleotides in length and are at least 60% reverse complementary to each other, wherein the second region of the target nucleic acid does not include any part of the first region of the target nucleic acid, and wherein the second region of the target nucleic acid does not include any part of a reverse complement of the first region of the target nucleic acid, and wherein hybridization of the GPS region and the GPS binding site positions the first strand primer binding site closer to the second region of the target nucleic acid, or
(iii) a modification in the reverse transcriptase template that disrupts a track of at least 4 consecutive nucleotides of the same base in the target nucleic acid.

### Zinc Finger Nucleases, TALENS, and Meganucleases

In some embodiments, the gene editing systems contemplated herein may comprise user-programmable DNA binding proteins that bind DNA through a specific amino acid sequence (i.e., are not reliant upon a guide RNA or nucleic acid programmability). Such enzymes include zinc finger nucleases and TALENS.

In some embodiments, the user-programmable nuclease is or comprises a TALE Nuclease, a TALE nickase, Zinc Finger (ZF) Nuclease, ZF Nickase, meganuclease, or a combination thereof. In some embodiments, the non-CRISPR/Cas sequence-specific nuclease is or includes two, three, four, or more of an independently selected TALE Nuclease, TALE nickase, Zinc Finger (ZF) Nuclease, ZF Nickase, Meganuclease, restriction enzymes or a combination thereof. In some embodiments, the combination is or comprises a TALE Nuclease/a ZF Nuclease; a TALE Nickase/a ZF nickase.

### F. TALENs

In some embodiments, the non-CRISPR/Cas sequence-specific nuclease is or comprises a TALE Nuclease (Transcription Activator-Like Effector Nucleases (TALEN)). TALENS are restriction enzymes engineered to cut specific target DNA sequences. TALENs comprise a TAL effector (TALE) DNA-binding domain (which binds at or close to the target DNA), fused to a DNA cleavage domain which cuts target DNA. TALEs are engineered to bind to practically any desired DNA sequence. Thus in some embodiments, the TALEN comprises an N-terminal capping region, a DNA binding domain which may comprise at least one or more TALE monomers or half-monomers specifically ordered to target the genomic locus of interest, and a C-terminal capping region, wherein these three parts are arranged in a predetermined N-terminus to C-terminus orientation. Optionally, the TALEN includes at least one or more regulatory or functional protein domains.

In some embodiments, the TALE monomers or half monomers may be variant TALE monomers derived from natural or wild type TALE monomers but with altered amino acids at positions usually highly conserved in nature, and in particular have a combination of amino acids as RVDs that do not occur in nature, and which may recognize a nucleotide with a higher activity, specificity, and/or affinity than a naturally occurring RVD. The variants may include deletions, insertions and substitutions at the amino acid level, and transversions, transitions and inversions at the nucleic acid level at one or more locations. The variants may also include truncations.

In some embodiments, the TALE monomer/half monomer variants include homologous and functional derivatives of the parent molecules. In some embodiments, the variants are encoded by polynucleotides capable of hybridizing under high stringency conditions to the parent molecule-encoding wild-type nucleotide sequences.

In some embodiments, the DNA binding domain of the TALE has at least 5 of more TALE monomers and at least one or more half-monomers specifically ordered or arranged to target a genomic locus of interest. The construction and generation of TALEs or polypeptides of the present disclosure may involve any of the methods known in the art.

Naturally occurring TALEs or “wild type TALEs” are nucleic acid binding proteins secreted by numerous species of proteobacteria. TALEs contain a nucleic acid binding domain composed of tandem repeats of highly conserved monomer polypeptides that are predominantly 33, 34 or 35 amino acids in length and that differ from each other mainly in amino acid positions 12 and 13. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain may comprise several repeats of TALE monomers and this may be represented as (X1-11-(X12X13)-X14-33 or 34 or 35) z, where z is optionally at least 5-40, such as 10-26.

The TALE monomers have a nucleotide binding affinity that is determined by the identity of the amino acids in its RVD. Polypeptide monomers with an RVD of NI preferentially bind to adenine (A), monomers with an RVD of NG preferentially bind to thymine (T), monomers with an RVD of HD preferentially bind to cytosine (C), monomers with an RVD of NN preferentially bind to both adenine (A) and guanine (G), monomers with an RVD of IG preferentially bind to T, monomers with an RVD of NS recognize all four base pairs and may bind to A, T, G or C. Thus, the number and order of the polypeptide monomer repeats in the nucleic acid binding domain of a TALE determines its nucleic acid target specificity. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In some embodiments, the TALE is a dTALE (or designerTALE), scc Zhang et al., Nature Biotechnology 29:149-153 (2011), incorporated herein by reference.

In some embodiments, the TALE monomer comprises an RVD of HN or NH that preferentially binds to guanine, and the TALEs have high binding specificity for guanine containing target nucleic acid sequences. In come embodiments, polypeptide monomers having RVDs RN, NN, NK, SN, NH, KN, HN, NQ, HH, RG, KH, RH and SS preferentially bind to guanine. In some embodiments, polypeptide monomers having RVDs RN, NK, NQ, HH, KH, RH, SS and SN preferentially bind to guanine. In some embodiments, polypeptide monomers having RVDs HH, KH, NH, NK, NQ, RH, RN and SS preferentially bind to guanine. In some embodiments, the RVDs that have high binding specificity for guanine are RN, NH RH and KH. In some embodiments, polypeptide monomers having an RVD of NV preferentially bind to adenine and guanine as do monomers having the RVD HN. Monomers having an RVD of NC preferentially bind to adenine, guanine and cytosine, and monomers having an RVD of S (or S*), bind to adenine, guanine, cytosine and thymine with comparable affinity. In more embodiments, monomers having RVDs of H*, HA, KA, N*, NA, NC, NS, RA, and S* bind to adenine, guanine, cytosine and thymine with comparable affinity. Such polypeptide monomers allow for the generation of degenerative TALEs able to bind to a repertoire of related, but not identical, target nucleic acid sequences.

In certain embodiments, the TALE polypeptide has a nucleic acid binding domain containing polypeptide monomers arranged in a predetermined N-terminus to C-terminus order such that each polypeptide monomer binds to a nucleotide of a predetermined target nucleic acid sequence, and where at least one of the polypeptide monomers has an RVD of HN or NH and preferentially binds to guanine, an RVD of NV and preferentially binds to adenine and guanine, an RVD of NC and preferentially binds to adenine, guanine and cytosine or an RVD of S and binds to adenine, guanine, cytosine and thymine.

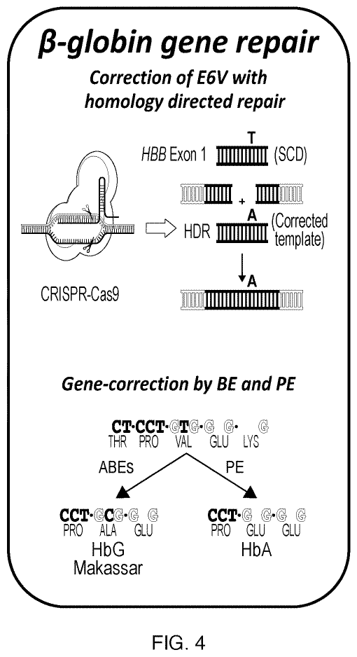

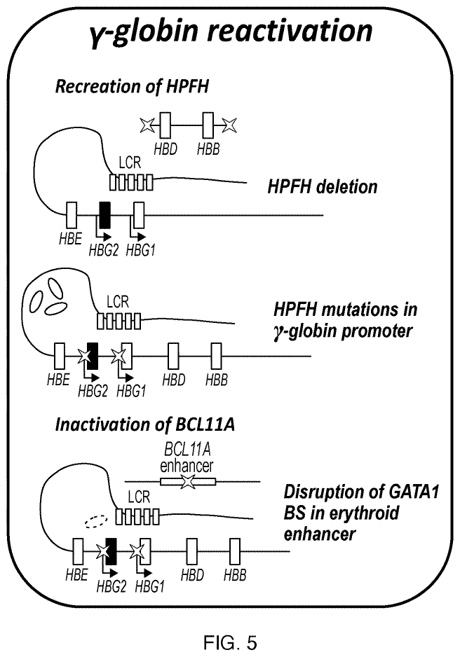

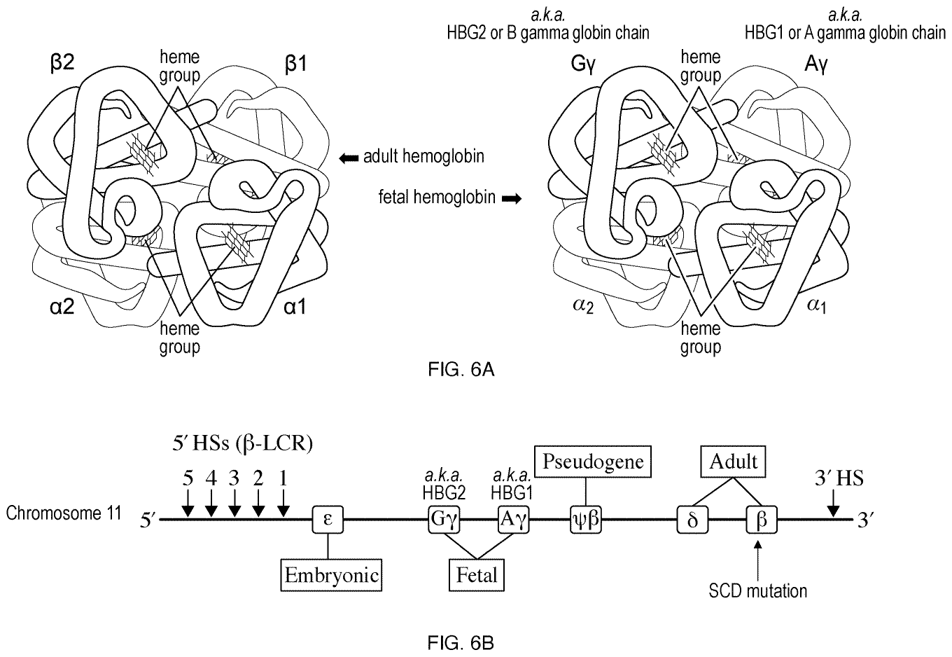

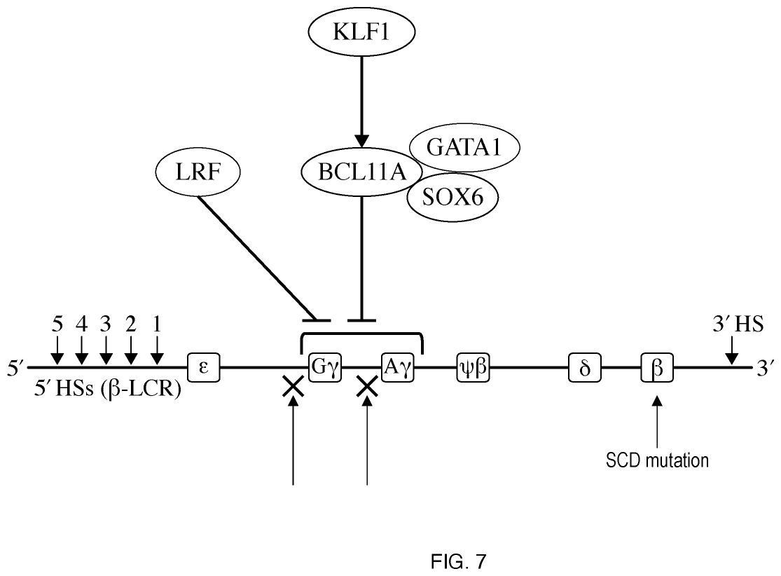

In some embodiments, each polypeptide monomer of the nucleic acid binding domain that binds to adenine has an RVD of NI, NN, NV, NC or S. In certain embodiments, each polypeptide monomer of the nucleic acid binding domain that binds to guanine has an RVD of HN, NH, NN, NV, NC or S. In certain embodiments, each polypeptide monomer of the nucleic acid binding domain that binds to cytosine has an RVD of HD, NC or S. In some embodiments, each polypeptide monomer that binds to thymine has an RVD of NG or S. In some embodiments, each polypeptide monomer of the nucleic acid binding domain that binds to adenine has an RVD of NI. In certain embodiments, each polypeptide monomer of the nucleic acid binding domain that binds to guanine has an RVD of HN or NH. In certain embodiments, each polypeptide monomer of the nucleic acid binding domain that binds to cytosine has an RVD of HD. In some embodiments, each polypeptide monomer that binds to thymine has an RVD of NG. In certain embodiments, the RVDs that have a specificity for adenine are NI, RI, KI, HI, and SI. In certain embodiments, the RVDs that have a specificity for adenine are HN, SI and RI, most preferably the RVD for adenine specificity is SI. In certain embodiments, the RVDs that have a specificity for thymine are NG, HG, RG and KG. In certain embodiments, the RVDs that have a specificity for thymine are KG, IIG and RG, most preferably the RVD for thymine specificity is KG or RG. In certain embodiments, the RVDs that have a specificity for cytosine are HD, ND, KD, RD, HH, YG and SD. In certain embodiments, the RVDs that have a specificity for cytosine are SD and RD. FIG. 4B of WO 2012/067428 provides representative RVDs and the nucleotides they target, the entire content of which is hereby incorporated herein by reference. In certain embodiments, the variant TALE monomers may comprise any of the RVDs that exhibit specificity for a nucleotide as depicted in FIG. 4A of WO2012/067428. All such TALE monomers allow for the generation of degenerative TALEs able to bind to a repertoire of related, but not identical, target nucleic acid sequences.

In certain embodiments, the RVD SH may have a specificity for G, the RVD IS may have a specificity for A, and the RVD IG may have a specificity for T.

In certain embodiments, the RVD NT may bind to G and A. In certain embodiments, the RVD NP may bind to A, T and C. In certain embodiments, at least one selected RVD may be NI, HD, NG, NN, KN, RN, NH, NQ, SS, SN, NK, KH, RH, HH, KI, HI, RI, SI, KG, HG, RG, SD, ND, KD, RD, YG, HN, NV, NS, HA, S*, N*, KA, H*, RA, NA or NC.

The predetermined N-terminal to C-terminal order of the one or more polypeptide monomers of the nucleic acid or DNA binding domain determines the corresponding predetermined target nucleic acid sequence to which the TALE or polypeptides of the present disclosure may bind. As used herein the monomers and at least one or more half monomers are

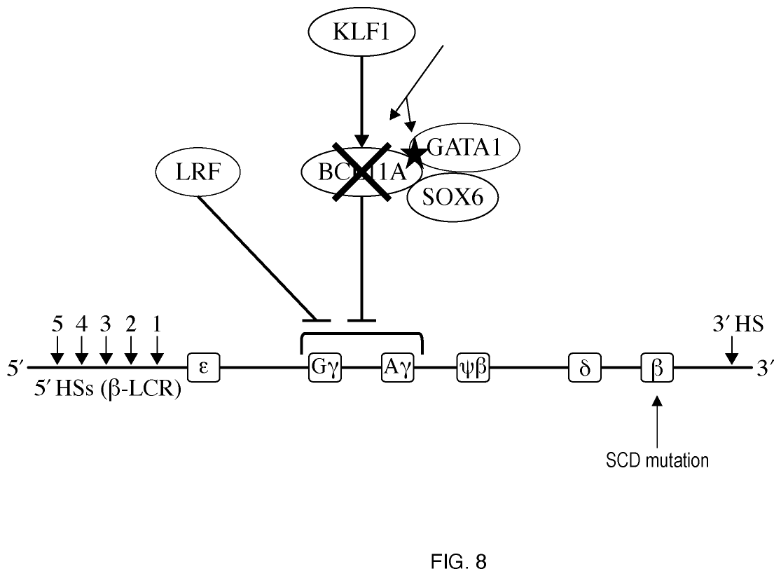

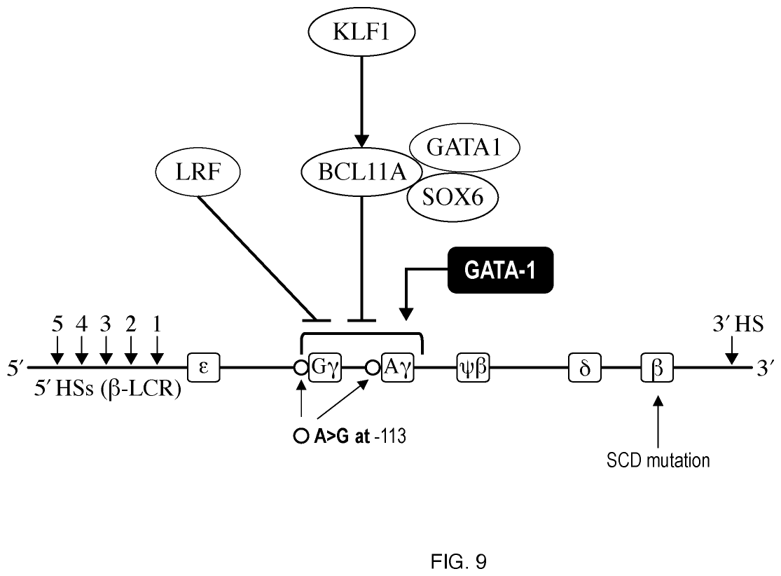

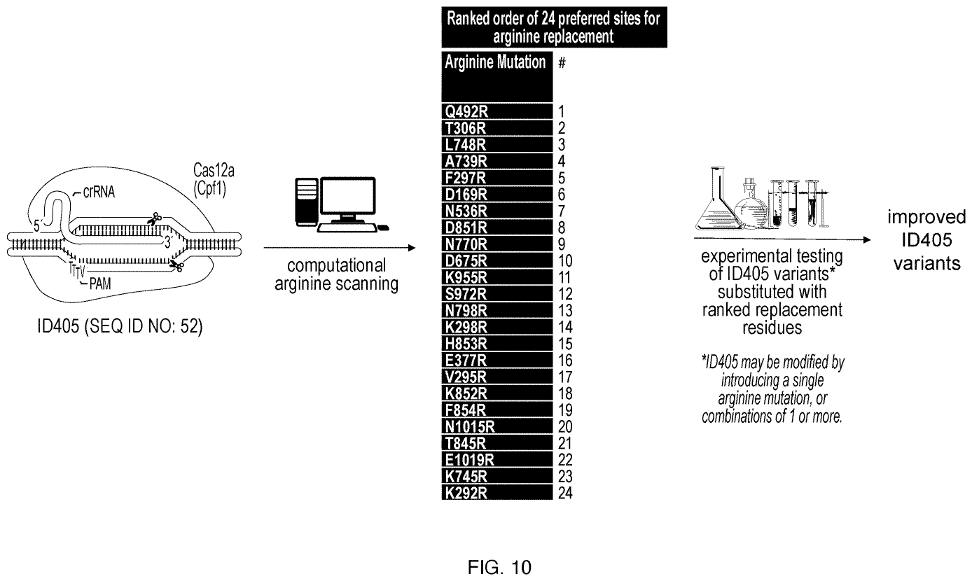

“specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thyminc (T) and polypeptides of the present disclosure may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8 of WO 2012/067428). Therefore, it follows that the length of the nucleic acid or DNA being targeted is equal to the number of full monomers plus two (see FIG. 44 of WO 2012/067428).

In certain embodiments, nucleic acid binding domains are engineered to contain 5, 6, 7, 8, 9, 10, 11, 12, 13, 14, 15, 16, 17, 18, 19, 20, 21, 22, 23, 24, 25, or more polypeptide monomers arranged in a N-terminal to C-terminal direction to bind to a predetermined 5, 6, 7, 8, 9, 10, 11, 12, 13, 14, 15, 16, 17, 18, 19, 20, 21, 22, 23, 24, 25 nucleotide length nucleic acid sequence.

In certain embodiments, nucleic acid binding domains are engineered to contain 5, 6, 7, 8, 9, 10, 11, 12, 13, 14, 15, 16, 17, 18, 19, 20, 21, 22, 23, 24, 25, 26 or more full length polypeptide monomers that are specifically ordered or arranged to target nucleic acid sequences of length 7, 8, 9, 10, 11, 12, 13, 14, 15, 16, 17, 18, 19, 20, 21, 22, 23, 24, 25, 26, 27 and 28 nucleotides, respectively. In certain embodiments, the polypeptide monomers are contiguous. In some embodiments, half-monomers may be used in the place of one or more monomers, particularly if they are present at the C-terminus of the TALE.

Polypeptide monomers are generally 33, 34 or 35 amino acids in length. With the exception of the RVD, the amino acid sequences of polypeptide monomers are highly conserved or as described herein, the amino acids in a polypeptide monomer, with the exception of the RVD, exhibit patterns that effect TALE activity, the identification of which may be used in preferred embodiments of the present disclosure.

In certain embodiments, when the DNA binding domain may comprise (X1-11-X12X13-X14-33 or 34 or 35) z, wherein XI-11 is a chain of 11 contiguous amino acids, wherein X12X13 is a repeat variable di-residue (RVD), wherein X14-33 or 34 or 35 is a chain of 21, 22 or 23 contiguous amino acids, wherein z is at least 5 to 26, then the preferred combinations of amino acids are LTLD or LTLA or LTQV at X1-4, or EQHG or RDHG at positions X30-33 or X31-34 or X32-35. Furthermore, other amino acid combinations of interest in the monomers are LTPD at X1-4 and NQALE at X16-20 and DHG at X32-34 when the monomer is 34 amino acids in length. When the monomer is 33 or 35 amino acids long, then the corresponding shift occurs in the positions of the contiguous amino acids NQALE and DHG. In certain embodiments, NOALE is at X15-19 or X17-21 and DHG is at X31-33 or X33-35.

In certain embodiments, amino acid combinations of interest in the monomers, are LTPD at X1-4 and KRALE at X16-20 and AHG at X32-34 or LTPE at X1-4 and KRALE at X16-20 and DHG at X32-34 when the monomer is 34 amino acids in length. When the monomer is 33 or 35 amino acids long, the corresponding shift occurs in the positions of the contiguous amino acids KRALE, AHG and DHG. In certain embodiments, the positions of the contiguous amino acids may be (LTPD at X1-4 and KRALE at X15-19 and AHG at X31-33) or (LTPE at X1-4 and KRALE at X15-19 and DIIG at X31-33) or (LTPD at X1-4 and KRALE at X17-21 and AIIG at X33-35) or (LTPE at X1-4 and KRALE at X17-21 and DHG at X33-35).

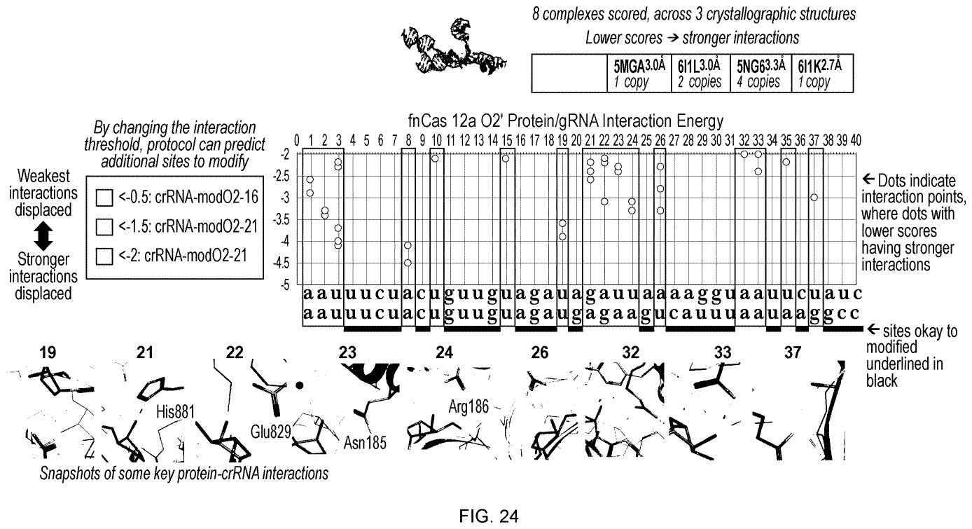

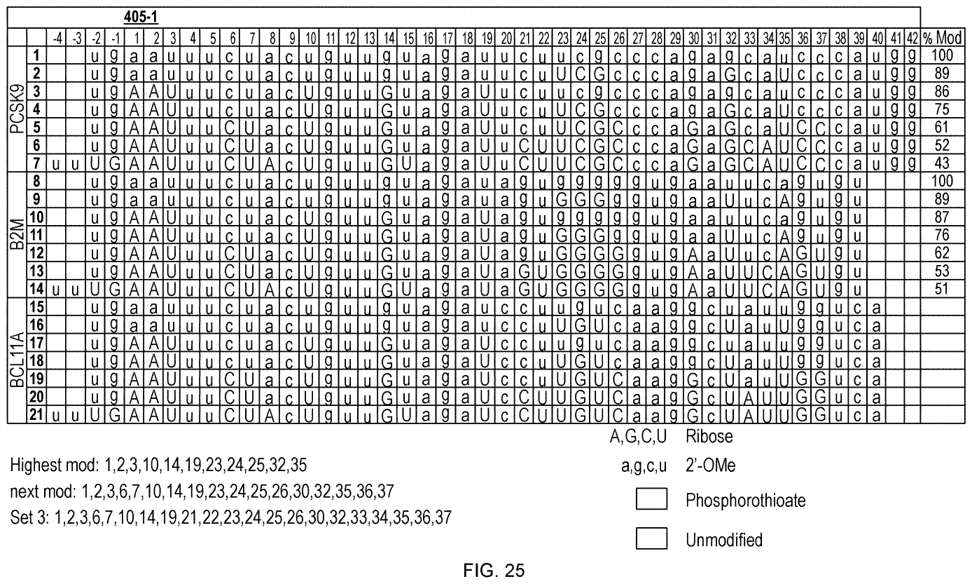

In certain embodiments, contiguous amino acids [NGKQALE] are present at positions X14-20 or X13-19 or X15-21. These representative positions put forward various embodiments of the present disclosure and provide guidance to identify additional amino acids of interest or combinations of amino acids of interest in all the TALE monomers (see FIGS. 24A-24F, and 25 of WO 2012/067428, which is incorporated by reference herein, in its entirety).

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the present disclosure.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE (including TALEs) polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. In certain embodiments, C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result. % homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Additional sequences for the conserved portions of polypeptide monomers and for N-terminal and C-terminal capping regions are included in the sequences with the following gene accession numbers: AAW59491.1, AAQ79773.2, YP_450163.1, YP_001912778.1, ZP_02242672.1, AAW59493.1, AAY54170.1, ZP_02245314.1, ZP_02243372.1, AAT46123.1, AAW59492.1, YP_451030.1, YP_001915105.1, ZP_02242534.1, AAW77510.1, ACD11364.1, ZP_02245056.1, ZP_02245055.1, ZP_02242539.1, ZP_02241531.1, ZP_02243779.1, AAN01357.1, ZP_02245177.1, ZP_02243366.1, ZP_02241530.1, AAS58130.3, ZP_02242537.1, YP_200918.1, YP_200770.1, YP_451187.1, YP_451156.1, AAS58127.2, YP_451027.1, UR_451025.1, AAA92974.1, UR_001913755.1, ABB70183.1, UR_451893.1, UR_450167.1, ABY60855.1, UR_200767.1, ZR_02245186.1, ZR_02242931.1, ZR_02242535.1, AAU54169.1, UR_450165.1, UR_001913452.1, AAS58129.3, ACM44927.1, ZR_02244836.1, AAT46125.1, UR_450161.1, ZR_02242546.1, AAT46122.1, UR_451897.1, AAF98343.1, UR_001913484.1, AAY54166.1, UR_001915093.1, UR_001913457.1, ZR_02242538.1, UR_200766.1, UR_453043.1, UR_001915089.1, UR_001912981.1, ZR_02242929.1, UR_001911730.1, UR_201654.1, UR_199877.1, ABB70129.1, UR_451696.1, UR_199876.1, AAS75145.1, AAT46124.1, UR_200914.1, UR 001915101.1, ZR_02242540.1, AAG02079.2, UR_451895.1, YP 451189.1, UR_200915.1, AAS46027.1, UR_001913759.1, UR_001912987.1, AAS58128.2, AAS46026.1, UR_201653.1, UR_202894.1, UR_001913480.1, ZR_02242666.1, R_001912775.1, ZR_02242662.1, AAS46025.1, AAC43587.1, BAA37119.1, NPJ544725.1, AB077779.1, BAA37120.1, ACZ62652.1, BAF46271.1, ACZ62653.1, NPJ544793.1, ABO77780.1, ZR_02243740.1, ZR_02242930.1, AAB69865.1, AAY54168.1, ZR_02245191.1, UR_001915097.1, ZR_02241539.1, UR_451158.1, BAA37121.1, UR_001913182.1, UR_200903.1, ZR_02242528.1, ZR_06705357.1, ZR_06706392.1, ADI48328.1, ZR_06731493.1, ADI48327.1, AB077782.1, ZR 06731656.1, NR_942641.1, AAY43360.1, ZR_06730254.1, ACN39605.1, UR_451894.1, UR_201652.1, UR_001965982.1, BAF46269.1, NPJ544708.1, ACN82432.1, AB077781.1, P14727.2, BAF46272.1, AAY43359.1, BAF46270.1, NR_644743.1, ABG37631.1, AAB00675.1, YP 199878.1, ZR_02242536.1, CAA48680.1, ADM80412.1, AAA27592.1, ABG37632.1, ABP97430.1, ZR_06733167.1, AAY43358.1, 2KQ5_A, BAD42396.1, ABO27075.1, UR_002253357.1, UR_002252977.1, ABO27074.1, ABO27067.1, ABO27072.1, ABO27068.1, UR_003750492.1, ABO27073.1, NR_519936.1, ABO27071.1, AB027070.1, and ABO27069.1, each of which is hereby incorporated by reference.

In some embodiments, the TALEs described herein also include a nuclear localization signal and/or cellular uptake signal. Such signals are known in the art and may target a TALE to the nucleus and/or intracellular compartment of a cell. Such cellular uptake signals include, but are not limited to, the minimal Tat protein transduction domain which spans residues 47-57 of the human immunodeficiency virus Tat protein.

In some embodiments, the TALEs described herein include a nucleic acid or DNA binding domain that is a non-TALE nucleic acid or a non-TALE DNA binding domain.

As used herein the term “non-TALE DNA binding domain” refers to a DNA binding domain that has a nucleic acid sequence corresponding to a nucleic acid sequence which is not substantially homologous to a nucleic acid that encodes for a TALE protein or fragment thereof, e.g., a nucleic acid sequence which is different from a nucleic acid that encodes for a TALE protein and which is derived from the same or a different organism.

In certain embodiments, the TALEs described herein include a nucleic acid or DNA binding domain that is linked to a non-TALE polypeptide.

A “non-TALE polypeptide” refers to a polypeptide having an amino acid sequence corresponding to a protein which is not substantially homologous to a TALE protein or fragment thereof, e.g., a protein which is different from a TALE protein and which is derived from the same or a different organism. In this context, the term “linked” is intended include any manner by which the nucleic acid binding domain and the non-TALE polypeptide could be connected to each other, including, for example, through peptide bonds by being part of the same polypeptide chain or through other covalent interactions, such as a chemical linker. The non-TALE polypeptide may be linked, for example to the N-terminus and/or C-terminus of the nucleic acid binding domain, may be linked to a C-terminal or N-terminal cap region, or may be connected to the nucleic acid binding domain indirectly.

In certain embodiments, the TALEs or polypeptides of the present disclosure comprise chimeric DNA binding domains. Chimeric DNA binding domains may be generated by fusing a full TALE (including the N- and C-terminal capping regions) with another TALE or non-TALE DNA binding domain such as zinc finger (ZF), helix-loop-helix, or catalytically-inactivated DNA endonucleases (e.g., EcoRI, meganucleases, etc.), or parts of TALE may be fused to other DNA binding domains. The chimeric domain may have novel DNA binding specificity that combines the specificity of both domains.

In certain embodiments, the TALE polypeptides of the present disclosure include a nucleic acid binding domain linked to the one or more effector domains. In certain embodiments, the effector domain is a nickase or nuclease.

### G. ZFNs

In certain embodiments, the sequence-specific nuclease is a zinc finger nuclease (ZFN), such as an artificial zinc-finger nuclease having arrays of zinc-finger (ZF) modules to target new DNA-binding sites in a target sequence (e.g., target sequence or target site in the genome). Each zinc finger module in a ZF array targets three DNA bases. A customized array of individual zinc finger domains is assembled into a ZF protein (ZFP). The resulting ZFP can be linked to a functional domain such as a nuclease.

ZF nucleases (ZFN) may be used as alternative programmable nucleases for use in retron-based editing in place of RNA-guide nucleases. ZFN proteins have been extensively described in the art, for example, in Carroll et al., “Genome Engineering with Zinc-Finger Nucleases,” Genetics, August 2011, Vol. 188:773-782; Durai et al., “Zinc finger nucleases: custom-designed molecular scissors for genome engineering of plant and mammalian cells,” Nucleic Acids Res, 2005, Vol. 33:5978-90; and Gaj et al., “ZFN, TALEN, and CRISPR/Cas-based methods for genome engineering,” Trends Biotechnol. 2013, Vol. 31:397-405, each of which are incorporated herein by reference in their entireties.

In certain embodiments, the ZF-linked nuclease is a catalytic domain of the Type IIS restriction enzyme FokI (see Kim et al., PNAS U.S.A. 91:883-887, 1994; Kim et al., PNAS U.S.A. 93:1156-1160, 1996, both incorporated herein by reference).

In certain embodiments, the ZFN comprises paired ZFN heterodimers, resulting in increased cleavage specificity and/or decreased off-target activity. In this embodiment, each ZFN in the heterodimer targets different nucleotide sequences separated by a short spacer (see Doyon et al., Nat. Methods 8:74-79, 2011, incorporated herein by reference).

In certain embodiments, the ZFN comprises a polynucleotide-binding domain (comprising multiple sequence-specific ZF modules) and a polynucleotide cleavage nickase domain.

In certain embodiments, the ZFs are engineered using libraries of two finger modules.

In certain embodiments, strings of two-finger units are used in ZFNs to improve DNA binding specificity from polyzinc finger peptides (see PNAS USA 98:1437-1441, incorporated herein by reference).

In certain embodiments, the ZFN has more than 3 fingers. In certain embodiments, the ZFN has 4, 5, or 6 fingers. In certain embodiments, the ZF modules in the ZFN are separated by one or more linkers to improve specificity.

In certain embodiments, the ZF of the ZFN includes substitutions in the dimer interface of the cleavage domain that prevent homodimerization between ZFs, but allow heterodimers to form.

In certain embodiments, the ZF of the ZFN has a design that retains activity while suppressing homodimerization.

In certain embodiments, the ZFN is any one of the ZF nucleases in Table 1 of Carroll et al., Genetics 188(4):773-782, 2011, incorporated herein by reference.

General principles and guidance for generating ZF, ZF arrays, and ZFN can be found in the art, such as the modular design (where the different modules can be rearranged and assembled into new combinations for new targets) of the ZF or ZF arrays in the ZFN as taught in Carroll et al., Nat. Protoc. 1:1329-1341, 2006 (incorporated herein by reference); the new three-finger sets for engineered ZFs generated by using partially randomized libraries; profiling the DNA-binding specificities of engineered Cys2His2 zinc finger domains using a rapid cell-based method (see Nucleic Acids Res. 35: e81, incorporated by reference). ZFs for certain DNA triplets that work well in neighbor combination are described in Sander et al., 2011. Selection-free zinc-finger-nuclease engineering by context-dependent assembly (CoDA) is taught in Nat. Methods 8:67-69). ToolGen describes the individual fingers in their collection that are best behaved in modular assembly (Kim et al., 2011). Preassembled zinc-finger arrays for rapid construction of ZFNs are taught in Nat. Methods 8:7.

Additional, non-limiting ZFs and AFNz that can be adapted for use in the instant disclosure include those described in WO2010/065123, WO2000/041566, WO2003/080809, WO2015/143046, WO2016/183298, WO2013/044008, WO2015/031619, WO2017/136049, WO2016/014794, WO2017/091512, WO1995/009233, WO2000/023464, WO2000/042219, WO2002/026960, WO2001/083793; U.S. Pat. Nos. 9,428,756, 9,145,565, 8,846,578, 8,524,874, 6,777,185, 6,599,692, 7,235,354, 6,503,717, 7,491,531, 7,943,553, 7,262,054, 8,680,021, 7,705,139, 7,273,923, 6,780,590, 6,785,613, 7,788,044, 7,177,766, 6,453,242, 6,794,136, 7,358,085, 8,383,766, 7,030,215, 7,013,219, 7,361,635, 7,939,327, 8,772,453, 9,163,245, 7,045,304, 8,313,925, 9,260,726, 6,689,558, 8,466,267, 7,253,273, 7,947,873, 9,388,426, 8,153,399, 8,569,253, 8,524,221, 7,951,925, 9,115,409, 8,772,008, 9,121,072, 9,624,498, 6,979,539, 9,491,934, 6,933,113, 9,567,609, 7,070,934, 9,624,509, 8,735,153, 9,567,573, 6,919,204, US2002-0081614, US2004-0203064, US2006-0166263, US2006-0292621, US2003-0134318, US2006-0294617, US2007-0287189, US2007-0065931, US2003-0105593, US2003-0108880, US2009-0305402, US2008-0209587, US2013-0123484, US2004-0091991, US2009-0305977, US2008-0233641, US2014-0287500, US2011-0287512, US2009-0258363, US2013-0244332, US2007-0134796, US2010-0256221, US2005-0267061, US2012-0204282, US2012-0252122, US2010-0311124, US2016-0215298, US2008-0031109, US2014-0017214, US2015-0267205, US2004-0235002, US2004-0204345, US2015-0064789, US2006-0063231, US2011-0265198, US2017-0218349, all incorporated herein by reference.

Polynucleotides and vectors capable of expressing one or more of the ZFNs are also provided herein, which can be part of the vector system of the present disclosure. The polynucleotides and vectors can be expressed in a cell, such as a eukaryotic cell, a mammalian cell, or a human cell. Suitable vectors, cells and expression systems are described in greater detail elsewhere herein, and can be suitable for use with the TALEs, the meganucleases, and the CRISPR-Cas nucleases.

### H. Meganucleases

In some embodiments, the gene editing system comprises meganucleases. Meganucleases are homing endonucleases discovered in yeast that recognize fairly long DNA sequences, and create double-strand breaks that are mended via stimulation of homologous recombination. Meganucleases are sequence-specific endonucleases that use large (recognition sites to generate accurate double-strand breaks (DSBs), promoting efficient gene targeting through homologous recombination (HR).

Meganuclease enzymes and editing systems comprising meganucleases have been described in the literature, including the following references, each of which are incorporated herein in their entireties by reference.
Khalil A M. The genome editing revolution: review. J Genet Eng Biotechnol. 2020 Oct. 29; 18(1):68. doi: 10.1186/s43141-020-00078-y. PMID: 33123803; PMCID: PMC7596157.
Lanigan T M, Kopera H C, Saunders T L. Principles of Genetic Engineering. Genes (Basel). 2020 Mar. 10; 11(3):291. doi: 10.3390/genes11030291. PMID: 32164255; PMCID: PMC7140808.
Arnould S, Delenda C, Grizot S, Desseaux C, Pâques F, Silva G H, Smith J. The I-Crel meganuclease and its engineered derivatives: applications from cell modification to gene therapy. Protein Eng Des Sel. 2011 January; 24(1-2):27-31. doi: 10.1093/protein/gzq083. Epub 2010 Nov. 3. PMID: 21047873.
Pâques F, Duchateau P. Meganucleases and DNA double-strand break-induced recombination: perspectives for gene therapy. Curr Gene Ther. 2007 February; 7(1):49-66. doi: 10.2174/156652307779940216. PMID: 17305528.
Zekonyte U, Bacman S R, Smith J, Shoop W, Pereira C V, Tomberlin G, Stewart J, Jantz D, Moraes C T. Mitochondrial targeted meganuclease as a platform to eliminate mutant mtDNA in vivo. Nat Commun. 2021 May 28; 12(1):3210. doi: 10.1038/s41467-021-23561-7. PMID: 34050192; PMCID: PMC8163834.

Meganuclease enzymes and editing systems comprising meganucleases have also been described in the patent literature, including the following references, each of which are incorporated herein in their entireties by reference.

| Publication | |
|---|---|
| Number | Title |
| WO2022226316A1 | COMPOSITIONS AND METHODS FOR GENERATING MALE STERILE |
| | PLANTS |
| WO2022226305A1 | ENGINEERED MEGANUCLEASES THAT TARGET HUMAN |
| | MITOCHONDRIAL GENOMES |
| WO2022226303A1 | ENGINEERED MEGANUCLEASES THAT TARGET HUMAN |
| | MITOCHONDRIAL GENOMES |
| WO2022165111A1 | MODULATION OF TGF BETA SIGNALING IN GENETICALLY-MODIFIED |
| | EUKARYOTIC CELLS |
| WO2022104062A1 | ENGINEERED MEGANUCLEASES HAVING SPECIFICITY FOR |
| | RECOGNITION SEQUENCES IN THE DYSTROPHIN GENE |
| WO2022076547A1 | LIPID NANOPARTICLE COMPOSITIONS |
| WO2022040582A1 | ENGINEERED MEGANUCLEASES HAVING SPECIFICITY FOR A |
| | RECOGNITION SEQUENCE IN THE TRANSTHYRETIN GENE |
| WO2022035793A1 | ANTIBODIES AND FRAGMENTS SPECIFIC FOR B-CELL MATURATION |
| | ANTIGEN AND USES THEREOF |
| WO2021231959A1 | METHODS FOR IMMUNOTHERAPY |
| WO2021231954A1 | METHODS FOR IMMUNOTHERAPY |
| WO2021231495A1 | TREATMENT OF RETINITIS PIGMENTOSA USING IMPROVED |
| | ENGINEERED MEGANUCLEASES |
| WO2021231259A1 | SELF-LIMITING VIRAL VECTORS ENCODING NUCLEASES |
| WO2021158915A1 | RECOMBINANT ADENO-ASSOCIATED VIRUS COMPOSITIONS AND |
| | METHODS FOR PRODUCING AND USING THE SAME |
| WO2021113765A1 | OPTIMIZED ENGINEERED MEGANUCLEASES HAVING SPECIFICITY |
| | FOR A RECOGNITION SEQUENCE IN THE HEPATITIS B VIRUS |
| | GENOME |
| WO2021113543A1 | METHODS FOR CANCER IMMUNOTHERAPY, USING |
| | LYMPHODEPLETION REGIMENS AND CD19, CD20 OR BCMA |
| | ALLOGENEIC CAR T CELLS |
| WO2021087305A1 | CD20 CHIMERIC ANTIGEN RECEPTORS AND METHODS OF USE FOR |
| | IMMUNOTHERAPY |
| WO2021035170A1 | COMPOSITIONS AND METHODS FOR TCR REPROGRAMMING USING |
| | FUSION PROTEINS |
| WO2021035054A1 | LYMPHODEPLETION DOSING REGIMENS FOR CELLULAR |
| | IMMUNOTHERAPIES |
| WO2021016608A1 | COMPOSITIONS AND METHODS FOR SEQUENTIAL STACKING OF |
| | NUCLEIC ACID SEQUENCES INTO A GENOMIC LOCUS |
| WO2020227534A1 | OPTIMIZATION OF ENGINEERED MEGANUCLEASES FOR |
| | RECOGNITION SEQUENCES |
| WO2020206248A1 | GENETICALLY-MODIFIED IMMUNE CELLS COMPRISING A |
| | MICRORNA-ADAPTED SHRNA (SHRNAMIR) |
| WO2020206231A1 | METHODS OF PREPARING POPULATIONS OF GENETICALLY- |
| | MODIFIED IMMUNE CELLS |
| WO2020146807A1 | GENETICALLY-MODIFIED CELLS COMPRISING A MODIFIED |
| | TRANSFERRIN GENE |
| WO2020132659A1 | GENETIC MODIFICATION OF THE HYDROXYACID OXIDASE 1 GENE |
| | FOR TREATMENT OF PRIMARY HYPEROXALURIA |
| WO2019200247A1 | OPTIMIZED ENGINEERED MEGANUCLEASES HAVING SPECIFICITY |
| | FOR A RECOGNITION SEQUENCE IN THE HEPATITIS B VIRUS |
| | GENOME |
| WO2019200122A1 | OPTIMIZED ENGINEERED NUCLEASES HAVING SPECIFICITY FOR |
| | THE HUMAN T CELL RECEPTOR ALPHA CONSTANT REGION GENE |
| WO2019089913A1 | ENGINEERED NUCLEASES THAT TARGET HUMAN AND CANINE |
| | FACTOR VIII GENES AS A TREATMENT FOR HEMOPHILIA A |
| WO2019070856A1 | MODIFIED EPIDERMAL GROWTH FACTOR RECEPTOR PEPTIDES FOR |
| | USE IN GENETICALLY-MODIFIED CELLS |
| WO2019005957A1 | GENETICALLY-MODIFIED T CELLS COMPRISING A MODIFIED |
| | INTRON IN THE T CELL RECEPTOR ALPHA GENE |
| WO2018208837A1 | NUCLEIC ACID MOLECULES ENCODING AN ENGINEERED ANTIGEN |
| | RECEPTOR AND AN INHIBITORY NUCLEIC ACID MOLECULE AND |
| | METHODS OF USE THEREOF |
| WO2018201144A1 | METHODS FOR REDUCING DNA-INDUCED CYTOTOXICITY AND |
| | ENHANCING GENE EDITING IN PRIMARY CELLS |
| WO2018195449A1 | ENGINEERED MEGANUCLEASES SPECIFIC FOR RECOGNITION |
| | SEQUENCES IN THE PCSK9 GENE |
| WO2018071849A2 | ENGINEERED MEGANUCLEASES SPECIFIC FOR RECOGNITION |
| | SEQUENCES IN THE HEPATITIS B VIRUS GENOME |
| WO2018067697A1 | CO-STIMULATORY DOMAINS FOR USE IN GENETICALLY-MODIFIED |
| | CELLS |
| WO2017192741A1 | ENGINEERED NUCLEASES USEFUL FOR TREATMENT OF |
| | HEMOPHILIA A |
| WO2017112859A1 | ENGINEERED MEGANUCLEASES WITH RECOGNITION SEQUENCES |
| | FOUND IN THE HUMAN BETA-2 MICROGLOBULIN GENE |
| WO2017062451A1 | GENETICALLY-MODIFIED CELLS COMPRISING A MODIFIED HUMAN |
| | T CELL RECEPTOR ALPHA CONSTANT REGION GENE |
| WO2017062439A1 | ENGINEERED MEGANUCLEASES WITH RECOGNITION SEQUENCES |
| | FOUND IN THE HUMAN T CELL RECEPTOR ALPHA CONSTANT |
| | REGION GENE |
| WO2017044649A1 | TREATMENT OF RETINITIS PIGMENTOSA USING ENGINEERED |
| | MEGANUCLEASES |
| WO2016205825A1 | SELF-LIMITING VIRAL VECTORS ENCODING NUCLEASES |
| WO2016179112A1 | PRECISE DELETION OF CHROMOSCOMAL SEQUENCES IN VIVO AND |
| | TREATMENT OF NUCLEOTIDE REPEAT EXPANSION DISORDERS |
| | USING ENGINEERED NUCLEASES |
| WO2015138739A2 | DYSTROPHIN GENE OXON DELETION USING ENGINEERED |
| | NUCLEASES |
| WO2012167192A2 | METHODS AND PRODUCTS FOR PRODUCING ENGINEERED |
| | MAMMALIAN CELL LINES WITH AMPLIFIED TRANSGENES |
| WO2010009147A1 | RECOGNITION SEQUENCES FOR I-CREI-DERIVED MEGANUCLEASES |
| | AND USES THEREOF |
| WO2009134714A2 | FUSION MOLECULES OF RATIONALLY-DESIGNED DNA-BINDING |
| | PROTEINS AND EFFECTOR DOMAINS |
| WO2009114321A2 | RATIONALLY-DESIGNED MEGANUCLEASES FOR MAIZE GENOME |
| | ENGINEERING |
| WO2009076292A2 | RATIONALLY-DESIGNED MEGANUCLEASES WITH RECOGNITION |
| | SEQUENCES FOUND IN DNASE HYPERSENSITIVE REGIONS OF THE |
| | HUMAN GENOME |
| WO2009059195A2 | RATIONALLY-DESIGNED SINGLE-CHAIN MEGANUCLEASES WITH |
| | NON-PALINDROMIC RECOGNITION SEQUENCES |
| WO2007047859A2 | RATIONALLY-DESIGNED MEGANUCLEASES WITH ALTERED |
| | SEQUENCE SPECIFICITY AND DNA-BINDING AFFINITY |
| US20130340121A1 | Temperature-Dependent Meganuclease Activity |

### Gene Editor Accessory Proteins

In other aspects, the gene editing systems described herein may comprise one or more additional accessory proteins having genome modifying functions, including recombinases, invertases, nucleases, polymerases (e.g., reverse transcriptases), ligases, deaminases, transposases, or DNA binding domains. In various embodiments, the accessory proteins may be provided separately.

In other embodiments, the accessory proteins may be fused to another component of a given gene editing system, such as a CRISPR-Cas9, through a linker.

### Guide RNA Components

### I. Guide RNAs

The present disclosure further provides guide RNAs for use in accordance with the disclosed nucleic acid programmable DNA binding proteins (e.g., Cas9) for use in methods of editing. The disclosure provides guide RNAs that are designed to recognize target sequences. Such gRNAs may be designed to have guide sequences (or “spacers”) having complementarity to a target sequence. Such gRNAs may be designed to have not only a guide sequences having complementarity to a target sequence to be edited, but also to have a backbone sequence that interacts specifically with the nucleic acid programmable DNA binding protein.

In some embodiments, the guide RNA may be 15-100 nucleotides in length and comprise a sequence of at least 10, at least 15, or at least 20 contiguous nucleotides that is complementary to a target nucleotide sequence. The guide RNA may comprise a spacer sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target nucleotide sequence. In some cases, the guide sequence has a length in a range of from 17-30 nucleotides (nt) (e.g., from 17-25, 17-22, 17-20, 19-30, 19-25, 19-22, 19-20, 20-30, 20-25, or 20-22 nt). In some cases, the guide sequence has a length in a range of from 17-25 nucleotides (nt) (e.g., from 17-22, 17-20, 19-25, 19-22, 19-20, 20-25, or 20-22 nt). In some cases, the guide sequence has a length of 17 or more nt (e.g., 18 or more, 19 or more, 20 or more, 21 or more, or 22 or more nt; 19 nt, 20 nt, 21 nt, 22 nt, 23 nt, 24 nt, 25 nt, etc.). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt.

In some cases, the spacer sequence has a length of from 15 to 50 nucleotides (e.g., from 15 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt).

A subject guide RNA can interact with a target nucleic acid (e.g., double stranded DNA (dsDNA), single stranded DNA (ssDNA), single stranded RNA (ssRNA), or double stranded RNA (dsRNA)) in a sequence-specific manner via hybridization (i.e., base pairing).

The guide RNA can be modified to hybridize to any desired target sequence (e.g., while taking the PAM into account, e.g., when targeting a dsDNA target) within a target nucleic acid (e.g., a eukaryotic target nucleic acid such as genomic DNA). In some cases, the percent complementarity between the spacer sequence of the guide and the target site of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the spacer and the target site of the target nucleic acid is 80% or more (e.g., 85% or more, 90% or more, 95% or more, 97% or more, 98% or more, 99% or more, or 100%). In some cases, the percent complementarity between the spacer and the target site of the target nucleic acid is 90% or more (e.g., 95% or more, 97% or more, 98% or more, 99% or more, or 100%). In some cases, the percent complementarity between the spacer and the target site of the target nucleic acid is 100%.

In some cases, the percent complementarity between the spacer sequence and the target site of the target nucleic acid is 100% over an at least 5-nucleotide contiguous region of the spacer. In some cases, the percent complementarity between the guide sequence and the target site of the target nucleic acid over an at least 6-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 7-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 8-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 9-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 10-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 11-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 12-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 13-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 14-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 15-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 16-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 17-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 18-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 19-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 20-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 21-nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 22-nucleotide contiguous region of the spacer is 60% or more (e.g., 70% or more, 75% or more, 80% or more, 85% or more, 90% or more, 95% or more, 97% or more, 98% or more, 99% or more, or 100%).

In some cases, the percent complementarity between the spacer sequence and the target site of the target nucleic acid is 100% over an at least 5-10 nucleotide contiguous region of the spacer. In some cases, the percent complementarity between the guide sequence and the target site of the target nucleic acid over an at least 6-11 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 7-12 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 8-13 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 9-14 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 10-15 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 11-16 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 12-17 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 13-18 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 14-19 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 15-20 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 16-21 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 17-22 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 18-23 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 19-24 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 20-25 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 21-26 nucleotide contiguous region of the spacer is 60% or more (e.g., 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid over an at least 22-27 nucleotide contiguous region of the spacer is 60% or more (e.g., 70% or more, 75% or more, 80% or more, 85% or more, 90% or more, 95% or more, 97% or more, 98% or more, 99% or more, or 100%).

In various embodiments, the guide RNAs may have a scaffold or core region that complexes with a cognate nucleic acid programmable DNA binding protein (e.g., CRISPR Cas9 or Cas12a). In some cases, a guide scaffold can have two stretches of nucleotides that are complementary to one another and hybridize to form a double stranded RNA duplex (dsRNA duplex). Thus, in some cases, the protein binding segment of a guide RNA includes a dsRNA duplex. In some embodiments, the dsRNA duplex region includes a range of from 5-25 base pairs (bp) (e.g., from 5-22, 5-20, 5-18, 5-15, 5-12, 5-10, 5-8, 8-25, 8-22, 8-18, 8-15, 8-12, 12-25, 12-22, 12-18, 12-15, 13-25, 13-22, 13-18, 13-15, 14-25, 14-22, 14-18, 14-15, 15-25, 15-22, 15-18, 17-25, 17-22, or 17-18 bp, e.g., 5 bp, 6 bp, 7 bp, 8 bp, 9 bp, 10 bp, etc.). In some cases, the dsRNA duplex region includes a range of from 6-15 base pairs (bp) (e.g., from 6-12, 6-10, or 6-8 bp, e.g., 6 bp, 7 bp, 8 bp, 9 bp, 10 bp, etc.). In some cases, the duplex region includes 5 or more bp (e.g., 6 or more, 7 or more, or 8 or more bp). In some cases, the duplex region includes 6 or more bp (e.g., 7 or more, or 8 or more bp). In some cases, not all nucleotides of the duplex region are paired, and therefore the duplex forming region can include a bulge. The term “bulge” herein is used to mean a stretch of nucleotides (which can be one nucleotide) that do not contribute to a double stranded duplex, but which are surround 5′ and 3′ by nucleotides that do contribute, and as such a bulge is considered part of the duplex region. In some cases, the dsRNA includes 1 or more bulges (e.g., 2 or more, 3 or more, 4 or more bulges). In some cases, the dsRNA duplex includes 2 or more bulges (e.g., 3 or more, 4 or more bulges). In some cases, the dsRNA duplex includes 1-5 bulges (e.g., 1-4, 1-3, 2-5, 2-4, or 2-3 bulges).

Thus, in some cases, the stretches of nucleotides that hybridize to one another to form the dsRNA duplex in a guide scaffold region have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 85%-100% complementarity (e.g., 90%-100%, 95%-100% complementarity) with one another. In some cases, the stretches of nucleotides that hybridize to one another to form the dsRNA duplex have 70%-95% complementarity (e.g., 75%-95%, 80%-95%, 85%-95%, 90%-95% complementarity) with one another. In other words, in some cases, the dsRNA duplex includes two stretches of nucleotides that have 70%-100% complementarity (e.g., 75%-100%, 80%-10%, 85%-100%, 90%-100%, 95%-100% complementarity) with one another. In some cases, the dsRNA duplex includes two stretches of nucleotides that have 85%-100% complementarity (e.g., 90%-100%, 95%-100% complementarity) with one another. In some cases, the dsRNA duplex includes two stretches of nucleotides that have 70%-95% complementarity (e.g., 75%-95%, 80%-95%, 85%-95%, 90%-95% complementarity) with one another.

In various embodiments, the scaffold region of a guide RNA can also include one or more (1, 2, 3, 4, 5, etc.) mutations relative to a naturally occurring scaffold region. For example, in some cases a base pair can be maintained while the nucleotides contributing to the base pair from each segment can be different. In some cases, the duplex region of a subject guide RNA includes more paired bases, less paired bases, a smaller bulge, a larger bulge, fewer bulges, more bulges, or any convenient combination thereof, as compared to a naturally occurring duplex region (of a naturally occurring guide RNA).

Examples of various guide RNAs can be found in the art, and in some cases variations similar to those introduced into Cas9 guide RNAs can also be introduced into guide RNAs of the present disclosure (e.g., mutations to the dsRNA duplex region, extension of the 5′ or 3′ end for added stability for to provide for interaction with another protein, and the like). For example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et. al, Nucleic Acids Rcs. 2013 Nov. 1; 41(20):c187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et. at., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

### J. Guide RNA Modifications

In one embodiment, the guide RNAs (including pegRNAs) contemplated herein comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the present disclosure, a guide RNA (including pegRNA) component nucleic acid comprises ribonucleotides and non-ribonucleotides. In one such embodiment, a guide RNA (including pegRNA) component comprises one or more ribonucleotides and one or more deoxyribonucleotides. In an embodiment of the present disclosure, the guide RNA (including pegRNA) component comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA).

Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of coRNA chemical modifications include, without limitation, incorporation of 2′-O-methyl (M), 2′-O-methyl 3′phosphorothioate (MS), S-constrained ethyl (cEt), or 2′-O-methyl 3′ thioPACE (MSP) at one or more terminal nucleotides. Such chemically modified oRNA components can comprise increased stability and increased activity as compared to unmodified oRNA components, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9):985-989; Li et al., Nature Biomedical Engineering, 2017, 1, 0066 D01: 10.1038/s41551-017-0066). In one embodiment, the 5′ and/or 3′ end of a guide RNA (including pegRNA) component is modified by a variety of functional moieties including fluorescent dyes, polyethylene glycol, cholesterol, proteins, or detection tags. (See Kelly et al., 2016, J. Biotech. 233:74-83). In one embodiment, a guide RNA (including pegRNA) component comprises ribonucleotides in a region that binds to a target sequence and one or more deoxyribonucletides and/or nucleotide analogs in a region that binds to a nucleic acid programmable DNA binding protein (e.g., Cas9 nickase).

In an embodiment, deoxyribonucleotides and/or nucleotide analogs are incorporated in engineered guide RNA (including pegRNA) component structures. In one embodiment, 3-5 nucleotides at either the 3′ or the 5′ end of a guide RNA (including pegRNA) component is chemically modified. In one embodiment, only minor modifications are introduced in the seed region, such as 2′-F modifications. In one embodiment, 2′-F modification is introduced at the 3′ end of a guide RNA (including pegRNA) component. In one embodiment, three to five nucleotides at the 5′ and/or the 3′ end of the reRNA component are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33(9): 985-989). In one embodiment, all of the phosphodiester bonds of a guide RNA (including pegRNA) component are substituted with phosphorothioates (PS) for enhancing levels of gene disruption. In one embodiment, more than five nucleotides at the 5′ and/or the 3′ end of the guide RNA (including pegRNA) component are chemically modified with 2′-O-Me, 2′-F or S-constrained ethyl (cEt). Such chemically modified guide RNA (including pegRNA) component can mediate enhanced levels of gene disruption (see Ragdarm et al., 0215, PNAS, E7110-E7111). In an embodiment of the present disclosure, a guide RNA (including pegRNA) component is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide RNA (including pegRNA) component by a linker, such as an alkyl chain. In one embodiment, the chemical moiety of the modified nucleic acid component can be used to attach the guide RNA (including pegRNA) component to another molecule, such as DNA, RNA, protein, or nanoparticles. Such chemically modified guide RNA (including pegRNA) component can be used to identify or enrich cells generically edited by a gene editing system described herein.

Other guide RNA (including pegRNA) modifications are described in Kim, D. Y., Lee, J. M., Moon, S. B. et al. Efficient CRISPR editing with a hypercompact Cas12f1 and engineered guide RNAs delivered by adeno-associated virus. Nat Biotechnol 40, 94-102 (2022).

Accordingly, in various aspects of the present disclosure, the guide RNA (including pegRNA) are modified in one or more locations within the molecule. MS1, an internal penta (uridinylate) (UUUUU) sequence in the tracrRNA; MS2, the 3′ terminus of the crRNA; MS3, the ‘stem 1’ region of the tracrRNA; MS4, the tracrRNA-crRNA complementary region; and MS5, the ‘stem 2’ region of the tracrRNA.

Various aspects of the present disclosure provide methods and compositions for improved guide RNA (including pegRNA) stability via chemical modifications. Braasch, D. A., Jensen, S., Liu, Y., Kaur, K., Arar, K., White, M. A., et al. (2003). RNA interference in mammalian cells by chemically-modified RNA. Biochemistry 42, 7967-7975. doi: 10.1021/bi0343774. Chiu, Y. L., and Rana, T. M. (2003). siRNA function in RNAi: a chemical modification analysis. RNA 9, 1034-1048. doi: 10.1261/rna.5103703. Behlke, M. A. (2008). Chemical modification of siRNAs for in vivo use. Oligonucleotides 18, 305-319. doi: 10.1089/oli.2008.0164. Bennett, C. F., and Swayze, E. E. (2010). RNA targeting therapeutics: molecular mechanisms of antisense oligonucleotides as a therapeutic platform. Annu. Rev. Pharmacol. Toxicol. 50, 259-293. doi: 10.1146/annurev.pharmtox.010909.105654. Deleavey, G. F., and Damha, M. J. (2012). Designing chemically modified oligonucleotides for targeted gene silencing. Chem. Biol. 19, 937-954. doi: 10.1016/j.chembiol.2012.07.011. Lennox, K. A., and Behlke, M. A. (2020). Chemical modifications in RNA interference and CRISPR/Cas genome editing reagents. Methods Mol. Biol. 2115, 23-55. doi: 10.1007/978-1-0716-0290-4_2.

For instance, Hendel et al. improved guide RNA stability by chemically modifying gRNA ends to reduce degradation by exonucleases, RNA nuclease. Hendel, A., Bak, R. O., Clark, J. T., Kennedy, A. B., Ryan, D. E., Roy, S., et al. (2015a). Chemically modified guide RNAs enhance CRISPR-Cas genome editing in human primary cells. Nat. Biotechnol. 33, 985-989. doi: 10.1038/nbt.3290. Chemical modifications of gRNAs may enable more efficient and safer gene-editing in primary cells suitable for clinical applications.

A review of types of chemical modifications are provided in Allen, Daniel et al. “Using Synthetically Engineered Guide RNAs to Enhance CRISPR Genome Editing Systems in Mammalian Cells.” Frontiers in genome editing vol. 2 617910. 28 Jan. 2021, doi: 10.3389/fgeed.2020.617910.

Accordingly, in various embodiments of the present disclosure, the genome editing system comprising a guide RNA (including pegRNA) and further comprises one or more chemical modifications selected from, but not limited to the modifications in the above table.

In exemplary embodiments, chemical modifications to the guide RNA (including pegRNA) include modifications on the ribose rings and phosphate backbone of guide RNA (including pegRNA) and modifications at the 2′OH include 2′-O-Me, 2′-F, and 2′F-ANA. More extensive ribose modifications include 2′F-4′-Cα-OMe and 2′,4′-di-Cα-OMe combine modification at both the 2′ and 4′ carbons. Phosphodiester modifications include sulfide-based Phosphorothioate (PS) or acetate-based phosphonoacetate alterations. Combinations of the ribose and phosphodiester modifications have given way to formulations such as 2′-O-methyl 3′phosphorothioate (MS), or 2′-O-methyl-3′-thioPACE (MSP), and 2′-O-methyl-3′-phosphonoacetate (MP) RNAs. Locked and unlocked nucleotides such as locked nucleic acid (LNA), bridged nucleic acids (BNA), S-constrained ethyl (cEt), and unlocked nucleic acid (UNA) are examples of sterically hindered nucleotide modifications. Modifications to make a phosphodiester bond between the 2′ and 5′ carbons (2′,5′-RNA) of adjacent RNAs as well as a butane 4-carbon chain link between adjacent RNAs have been described.

### F. Additional Components and Aspects

In addition to the above LNPs and cargoes, including (A) nucleic acid payloads, (B) linear mRNA payloads, circular mRNA payloads, and (D) gene editing systems, the present disclosure provides additional optional LNP cargo components and tools that may be included as appropriate in the LNP gene editing systems described herein. The following optional components and tools may be combined in any combination as appropriate depending upon the particular gene editing system being delivered by the herein disclosed LNP-based gene editing systems.

### Polypeptides, Peptides, and Proteins

The LNP-based nucleobase editing systems and therapeutics described herein comprise one or more RNA payloads (e.g., linear or circular mRNA) which may comprise one or more coding regions that encode one or more products of interest. The one or more coding regions may encode a polypeptide, peptide and/or protein. As used herein, the term “polypeptide” generally refers to polymers of amino acids linked by peptide bonds and embraces “protein” and “peptides.” Polypeptides for the present disclosure include all polypeptides, proteins and/or peptides known in the art. Non-limiting categories of polypeptides include antigens, antibodies, antibody fragments, cytokines, peptides, hormones, enzymes, oxidants, antioxidants, synthetic polypeptides, and chimeric polypeptides, receptor, enzymes, hormones, transcription factors, ligands, membrane transporters, structural proteins, nucleases, or a component, variant or fragment (e.g., a biologically active fragment) thereof.

As used herein, the term “peptide” generally refers to shorter polypeptides of about 50 amino acids or less. Peptides with only two amino acids may be referred to as “dipeptides.” Peptides with only three amino acids may be referred to as “tripeptides.” Polypeptides generally refer to polypeptides with from about 4 to about 50 amino acids. Peptides may be obtained via any method known to those skilled in the art. In some embodiments, peptides may be expressed in culture. In some embodiments, peptides may be obtained via chemical synthesis (e.g., solid phase peptide synthesis).

In some embodiments, the RNA payloads (e.g., linear and/or circular mRNA payloads encoding one or more encoded products of interest), e.g., the originator constructs and benchmark constructs described herein, may encode a simple protein which upon hydrolysis yields the amino acids and occasionally small carbohydrate compounds. Non-limiting examples of simple proteins include albumins, albuminoids, globulins, glutelins, histones and protamines.

In some embodiments, the RNA payloads (e.g., linear and/or circular mRNA payloads encoding one or more encoded products of interest), e.g., the originator constructs and benchmark constructs described herein, may encode a simple protein associated with a non-protein. Non-limiting examples of conjugated proteins include, glycoproteins, hemoglobins, lecithoproteins, nucleoproteins, and phosphoproteins.

In some embodiments, the RNA payloads (e.g., linear and/or circular mRNA payloads encoding one or more encoded products of interest), e.g., the originator constructs and benchmark constructs described herein, may encode a protein that is derived from a simple or conjugated protein by chemical or physical means. Non-limiting examples of derived proteins include denatured proteins and peptides.

In some embodiments, the polypeptide, protein or peptide may be unmodified.

In some embodiments, the polypeptide, protein or peptide may be modified. Types of modifications include, but are not limited to, phosphorylation, glycosylation, acetylation, ubiquitylation/sumoylation, methylation, palmitoylation, quinone, amidation, myristoylation, pyrrolidone carboxylic acid, hydroxylation, phosphopantetheine, prenylation, GPI anchoring, oxidation, ADP-ribosylation, sulfation, S-nitrosylation, citrullination, nitration, gamma-carboxyglutamic acid, formylation, hypusine, topaquinone (TPQ), bromination, lysine topaquinone (LTQ), tryptophan tryptophylquinone (TTQ), iodination, and cysteine tryptophylquinone (CTQ). In some aspects, the polypeptide, protein or peptide may be modified by a post-transcriptional modification which can affect its structure, subcellular localization, and/or function.

In some embodiments, the polypeptide, protein or peptide may be modified using phosphorylation. Phosphorylation, or the addition of a phosphate group to serine, threonine, or tyrosine residues, is one of most common forms of protein modification. Protein phosphorylation plays an important role in fine tuning the signal in the intracellular signaling cascades.

In some embodiments, the polypeptide, protein or peptide may be modified using ubiquitination which is the covalent attachment of ubiquitin to target proteins. Ubiquitination-mediated protein turnover has been shown to play a role in driving the cell cycle as well as in protein-degradation-independent intracellular signaling pathways.

In some embodiments, the polypeptide, protein or peptide may be modified using acetylation and methylation which can play a role in regulating gene expression. As a non-limiting example, the acetylation and methylation could mediate the formation of chromatin domains (e.g., euchromatin and heterochromatin) which could have an impact on mediating gene silencing.

In some embodiments, the polypeptide, protein or peptide may be modified using glycosylation. Glycosylation is the attachment of one of a large number of glycan groups and is a modification that occurs in about half of all proteins and plays a role in biological processes including, but not limited to, embryonic development, cell division, and regulation of protein structure. The two main types of protein glycosylation are N-glycosylation and O-glycosylation. For N-glycosylation the glycan is attached to an asparagine and for O-glycosylation the glycan is attached to a serine or threonine.

In some embodiments, the polypeptide, protein or peptide may be modified using sumoylation. Sumoylation is the addition of SUMOs (small ubiquitin-like modifiers) to proteins and is a post-translational modification similar to ubiquitination.

In other embodiments, the RNA payloads (e.g., linear and/or circular mRNA payloads encoding one or more encoded products of interest), e.g., the originator constructs and benchmark constructs described herein, may encode a therapeutic protein, such as those exemplified below.

In other embodiments, the RNA payloads (e.g., linear and/or circular mRNA payloads encoding one or more products of interest), e.g., the originator constructs and benchmark constructs described herein, may encode a gene editing system, such as those exemplified herein. As used herein, a “nucleobase editing system” or “gene editing system” (used interchangeably herein) is a protein, DNA, or RNA composition capable of making edits, modifications or alterations to one or more targeted genes of interest. According to the present disclosure, one or more nucleobase editing system currently being marketed or in development may be encoded by the RNA payloads (e.g., linear and/or circular mRNA payloads encoding one or more encoded products of interest) described herein of the present disclosure.

### Fusion Proteins

In some embodiments, the polypeptide products (e.g., nucleobase editing systems and/or therapeutic proteins) of the RNA payload disclosed herein may be in the form of a fusion protein. Thus, the encoded polypeptides may include two or more proteins (e.g., protein and/or protein fragment) joined together, e.g., by a linker. In some embodiments, the fusion partner can provide an additional function to the encode polypeptide product, such as, but not limited to intracellular targeting, signaling, enzymatic function, stability, scaffolds, enhanced immunogenicity (in the case where the polypeptide encoded by the RNA payload is a nucleobase editing system). The disclosure contemplates that the polypeptide products (e.g., nucleobase editing systems and/or therapeutic proteins) of the RNA payload disclosed herein may be fused to any useful fusion partner known in the art.

### Functional Domains

In some embodiments, the polypeptides encoded by the RNA payloads described herein may further comprise additional sequences or functional domains. For example, the nucleobase editing system polypeptides of the present disclosure may comprise one or more linker sequences. In some embodiments, the nucleobase editing system polypeptide may comprise a polypeptide tag, such as an affinity tag (chitin binding protein (CBP), maltose binding protein (MBP), glutathione-S-transferase (GST), SBP-tag, Strep-tag, AviTag, Calmodulin-tag); solubilization tag; chromatography tag (polyanionic amino acid tag, such as FLAG-tag); epitope tag (short peptide sequences that bind to high-affinity antibodies, such as V5-tag, Myc-tag, VSV-tag, Xpress tag, E-tag, S-tag, and HA-tag); fluorescence tag (e.g., GFP). In some embodiments, the nucleobase editing system peptide may comprise an amino acid tag, such as one or more lysines, histidines, or glutamates, which can be added to the polypeptide sequences (e.g., at the N-terminal or C-terminal ends). Lysines can be used to increase peptide solubility or to allow for biotinylation. Protein and amino acid tags are peptide sequences genetically grafted onto a recombinant protein. Sequence tags are attached to proteins for various purposes, such as peptide purification, identification, or localization, for use in various applications including, for example, affinity purification, protein array, western blotting, immunofluorescence, and immunoprecipitation. Such tags are subsequently removable by chemical agents or by enzymatic means, such as by specific proteolysis or intein splicing.

Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

### Codon Optimization

The LNP-based nucleobase editing systems and therapeutics described herein may comprise one or more RNA payloads (e.g., linear or circular mRNA) having nucleotide sequences which may be codon optimized.

For example, a nucleotide sequence (e.g., as part of an RNA payload) encoding a nucleobase editing system of the disclosure is codon optimized. Codon optimization methods are known in the art. For example, a protein encoding sequence of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In some embodiments, the protein encoding sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding a nucleobase editing enzyme). In some embodiments, a codon optimized sequence shares less than 90% sequence identity to a naturally-occurring or wild-type sequence (e.g., a naturally-occurring or wild-type mRNA sequence encoding a nucleobase editing enzyme). In some embodiments, a codon optimized sequence shares less than 85% sequence identity to a naturally-occurring or wild-type sequence (e.g., a naturally-occurring or wild-type mRNA sequence encoding a nucleobase editing enzyme). In some embodiments, a codon optimized sequence shares less than 80% sequence identity to a naturally-occurring or wild-type sequence (e.g., a naturally-occurring or wild-type mRNA sequence encoding a nucleobase editing enzyme). In some embodiments, a codon optimized sequence shares less than 75% sequence identity to a naturally-occurring or wild-type sequence (e.g., a naturally-occurring or wild-type mRNA sequence encoding a nucleobase editing enzyme).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a nucleobase editing enzyme). In some embodiments, a codon optimized sequence shares between 65% and 75% or about 80% sequence identity to a naturally-occurring or wild-type sequence (e.g., a naturally-occurring or wild-type mRNA sequence encoding a nucleobase editing enzyme).

When transfected into mammalian cells, the modified mRNA payloads have a stability of between 12-18 hours, or greater than 18 hours, e.g., 24, 36, 48, 60, 72, or greater than 72 hours.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Donor Templates

In one embodiment, the compositions and systems herein may further comprise one or more donor templates for use in editing. In some cases, the donor template may comprise one or more polynucleotides. In certain cases, the donor template may comprise coding sequences for one or more polynucleotides. The donor template may be a DNA template. It may be single stranded or double stranded. It may also be circular single or double stranded. It may also be linear single stranded or double stranded. Without being bound by theory, the donor template may become integrated into the genome after a targeted cut by the Cas12a gene editing system described herein through cellular repair machinery including HDR and NHEJ.

The donor template may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor template alters a stop codon in the target polynucleotide. For example, the donor template may correct a premature stop codon. The correction may be achieved by deleting the stop codon or introduces one or more mutations to the stop codon. In other example embodiments, the donor template addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor templates that encode gene or gene fragments that compensate for or override defective gene expression such that cell phenotypes associated with defective gene expression are eliminated or changed to a different or desired cellular phenotype.

In an embodiment of the present disclosure, the donor template may include, but not be limited to, genes or gene fragments, encoding proteins or RNA transcripts to be expressed, regulatory elements, repair templates, and the like. According to the present disclosure, the donor templates may comprise left end and right end sequence elements that function with transposition components that mediate insertion.

In certain cases, the donor template manipulates a splicing site on the target polynucleotide. In some examples, the donor template disrupts a splicing site. The disruption may be achieved by inserting the polynucleotide to a splicing site and/or introducing one or more mutations to the splicing site. In certain examples, the donor template may restore a splicing site. For example, the polynucleotide may comprise a splicing site sequence.

The donor template to be inserted may has a size from 10 base pair or nucleotides to 50 kb in length, e.g., from 50 to 40k, from 100 and 30 k, from 100 to 10000, from 100 to 300, from 200 to 400, from 300 to 500, from 400 to 600, from 500 to 700, from 600 to 800, from 700 to 900, from 800 to 1000, from 900 to from 1100, from 1000 to 1200, from 1100 to 1300, from 1200 to 1400, from 1300 to 1500, from 1400 to 1600, from 1500 to 1700, from 600 to 1800, from 1700 to 1900, from 1800 to 2000 base pairs (bp) or nucleotides in length.

In some embodiments, the heterologous nucleic acid sequence is a donor DNA template that can be integrated into a host genome via HDR. In other embodiments, the heterologous nucleic acid sequence is a donor DNA template that can be integrated into a host genome via NHEJ.

In certain embodiments, the heterologous nucleic acid comprises or encodes a donor/template sequence, wherein the donor/template corrects/repairs/removes a mutation at the target genome site. For example, the mutation may be a mutated exon in a disease gene.

In certain embodiments, the donor/template may encode or comprises a functional DNA element, such as a promoter, an enhancer, a protein binding sequence, a methylation site, or a homology region for assisting gene editing, etc.

By “donor DNA” or “donor DNA template” it is meant a DNA segment (can be single stranded or double stranded DNA) to be inserted at a site cleaved by a gene-editing nuclease (e.g., a Cas12a nuclease) (e.g., after dsDNA cleavage, after nicking a target DNA, after dual nicking a target DNA, and the like). The donor DNA template can contain sufficient homology to a genomic sequence at the target site, e.g., 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. In the case of repair by NHEJ, no homology is needed on the donor DNA template against the site to which it targets editing.

Approximately 25, 50, 100, or 200 nucleotides, or more than 200 nucleotides, of sequence homology between a donor DNA template and a genomic sequence (or any integral value between 10 and 200 nucleotides, or more) can support homology-directed repair. Donor DNA template can be of any length, e.g., 50 nucleotides or more, 100 nucleotides or more, 250 nucleotides or more, 500 nucleotides or more, 1000 nucleotides or more, 5000 nucleotides or more, etc. A suitable donor DNA template can be from 50 nucleotides to 100 nucleotides, from 100 nucleotides to 500 nucleotides, from 500 nucleotides to 1000 nucleotides, from 1000 nucleotides to 5000 nucleotides, or from 5000 nucleotides to 10,000 nucleotides, or more than 10,000 nucleotides, in length.

As noted above, in some embodiments, the donor DNA template comprises a first homology arm and a second homology arm. The first homology arm is at or near the 5′ end of the donor DNA; and comprises a nucleotide sequence that is at least partially complementary to a first nucleotide sequence in a target nucleic acid. The second homology arm is at or near the 3′ end of the donor DNA; and comprises a nucleotide sequence that is at least partially complementary to a second nucleotide sequence in the target nucleic acid. The first and second homology arms can each independently have a length of from about 10 nucleotides to 400 nucleotides; e.g., from 10 nucleotides (nt) to 15 nt, from 15 nt to 20 nt, from 20 nt to 25 nt, from 25 nt to 30 nt, from 30 nt to 35 nt, from 35 nt to 40 nt, from 40 nt to 45 nt, from 45 nt to 50 nt, from 50 nt to 75 nt, from 75 nt to 100 nt, from 100 nt to 125 nt, from 125 nt to 150 nt, from 150 nt to 175 nt, from 175 nt to 200 nt, from 200 nt to 225 nt, from 225 nt to 250 nt, from 250 nt to 275 nt, from 275 nt to 300 nt, from 325 nt to 350 nt, from 350 nt to 375 nt, or from 375 nt to 400 nt.

In certain embodiments, the donor DNA template is used for editing the target nucleotide sequence. In certain embodiments, the donor DNA template comprises one or more mutations to be introduced into the target polynucleotide. Examples of such mutations include substitutions, deletions, insertions, or a combination thereof. In certain embodiments, the mutation causes a shift in an open reading frame on the target polynucleotide. In certain embodiments, the donor polynucleotide alters a stop codon in the target polynucleotide. In certain embodiments, the donor polynucleotide corrects a premature stop codon. The correction can be achieved by deleting the stop codon, or by introducing one or more sequence changes to alter the stop codon to a codon. In certain embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment includes a fragment less than the entire copy of a gene but otherwise provides sufficient nucleotide sequence to restore the functionality of a wild type gene or non-coding regulatory sequence (e.g., sequences encoding long non-coding RNA).

In certain embodiments, the donor DNA template may be used to replace a single allele of a defective gene or defective fragment thereof. In another embodiment, the donor DNA template is used to replace both alleles of a defective gene or defective gene fragment. A “defective gene” or “defective gene fragment” is a gene or portion of a gene that when expressed, fails to generate a functioning protein or non-coding RNA with functionality of the corresponding wild-type gene.

In certain example embodiments, these defective genes may be associated with one or more disease phenotypes. In certain example embodiments, the defective gene or gene fragment is not replaced but the heterologous nucleic acid is used to insert donor polynucleotides that encode gene or gene fragments that compensate for or override defective gene expression such that cell phenotypes associated with defective gene expression are eliminated or changed to a different or desired cellular phenotype. This can be achieved by including the coding sequence of a therapeutic protein, such as a therapeutic antibody or functional fragment thereof, or a wild-type version of a defective protein associated with one or more disease phenotypes.

In certain embodiments, the donor may include, but not be limited to, genes or gene fragments, encoding proteins or RNA transcripts to be expressed, regulatory elements, repair templates, and the like. According to the present disclosure, the donor polynucleotides may comprise left end and right end sequence elements that function with transposition components that mediate insertion.

In certain embodiments, the donor DNA template manipulates a splicing site on the target polynucleotide. In certain embodiments, the donor DNA template disrupts a splicing site. The disruption may be achieved by inserting the polynucleotide to a splicing site and/or introducing one or more mutations to the splicing site. In certain embodiments, the donor polynucleotide may restore a splicing site. For example, the polynucleotide may comprise a splicing site sequence.

In certain embodiments, the donor DNA template to be inserted has a size from 10 bp to 50 kb in length, e.g., from 50 bp to ~40 kb, from 100 bp to ~30 kb, from 100 bp to ~10 kb, from 100 bp to 300 bp, from 200 bp to 400 bp, from 300 bp to 500 bp, from 400 bp to 600 bp, from 500 bp to 700 bp, from 600 bp to 800 bp, from 700 bp to 900 bp, from 800 bp to 1000 bp, from 900 bp to 1100 bp, from 1000 bp to 1200 bp, from 1100 bp to 1300 bp, from 1200 bp to 1400 bp, from 1300 bp to 1500 bp, from 1400 bp to 1600 bp, from 1500 bp to 1700 bp, from 1600 bp to 1800 bp, from 1700 bp to 1900 bp, from 1800 bp to 2000 bp nucleotides in length.

In certain embodiments, the homologous arm on one or both ends of the sequence to be inserted is independently about 20 bp, 40 bp, 60 bp, 80 bp, 100 bp, 120 bp, or 150 bp.

The first homology arm and the second homology arm of the donor DNA flank a nucleotide sequence (“a nucleotide sequence of interest” or “an intervening nucleotide sequence”) that is to be introduced into a target nucleic acid. The nucleotide sequence of interest can comprise: i) a nucleotide sequence encoding a polypeptide of interest; ii) a nucleotide sequence encoding an exon of a gene; iii) a promoter sequence; iv) an enhancer sequence; v) a nucleotide sequence encoding a non-coding RNA; or vi) any combination of the foregoing.

The donor DNA can provide for gene correction, gene replacement, gene tagging, transgene insertion, nucleotide deletion, gene disruption, gene mutation, etc. For example, the donor DNA can be used to add, e.g., insert or replace, nucleic acid material to a target DNA (e.g. to “knock in” a nucleic acid that encodes a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, enhancer, etc.), to modify a nucleic acid sequence (e.g., introduce a mutation), and the like. For example, the donor DNA can be used to modify DNA in a site-specific, i.e. “targeted”, way; for example gene knock-out, gene knock-in, gene editing, gene tagging, etc., as used in, for example, gene therapy, e.g. to treat a disease; or as an antiviral, antipathogenic, or anticancer therapeutic, the production of genetically modified organisms in agriculture, the large scale production of proteins by cells for therapeutic, diagnostic, or research purposes, the induction of pluripotent stem cells, biological research, the targeting of genes of pathogens for deletion or replacement, etc.

In some cases, the donor DNA comprises a nucleotide sequence encoding a polypeptide of interest. Polypeptides of interest include, e.g., a) functional versions of a polypeptide that comprises one or more amino acid substitutions, insertions, and/or deletions and that exhibits reduced function, e.g., where the reduced function is associated with or causes a pathological condition; b) fluorescent polypeptides; c) hormones; d) receptors for ligands; e) ion channels; f) neurotransmitters; g) and the like.

In some cases, the donor DNA comprises a nucleotide sequence that encodes a wild-type protein that is lacking in the recipient cell. In some cases, the donor DNA encodes a wild type factor (e.g. Factor VII, Factor VIII, Factor IX and the like) involved in coagulation. In some cases, the donor DNA comprises a nucleotide sequence that encodes a therapeutic antibody. In some cases, the donor DNA comprises a nucleotide sequence that encodes an engineered protein or receptor. In some cases, the engineered receptor is a T cell receptor (TCR), a natural killer (NK) receptor (NKR), or a B cell receptor (BCR). In some cases, the engineered TCR or NKR targets a cancer marker (e.g., a polypeptide that is expressed (e.g., over-expressed) on the surface of a cancer cell). In some cases, the donor DNA comprises a nucleotide sequence that encodes a chimeric antigen receptor (CAR). In some cases, the CAR targets a cancer marker. Donor DNAs encoding CAR, TCR, and/or NCR proteins may be folded into DNA origami structures (DNA nanostructures) and delivered into T cells or NK cells in vitro or in vivo.

Non-limiting examples of polypeptides that can be encoded by a donor DNA include, e.g., IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WIIITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag (x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C—C motif) ligand 2), LPL (lipoprotein lipase), vWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp (a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric Golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), IIMOX1 (heme oxygenase (decycling) 1), APOC3 (apolipoprotein C—III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member lib), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMAI (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C—C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C—X—C motif) receptor 4), PROC (protein C (inactivator of coagulation factors Va and Villa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid A1), KCNH2 (potassium voltage-gated channel, subfamily II (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomer ase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CAB INI (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp 1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member A1), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IE17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, A1 polypeptide), TTR (transthyretin), FABP4 (fatty acid binding protein 4, adipocyte), PON3 (paraoxonase 3), APOC1 (apolipoprotein C—I), INSR (insulin receptor), TNFRSF1B (tumor necrosis factor receptor superfamily, member IB), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THIBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide A1), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member A1), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac1)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin LI), PCNA (proliferating cell nuclear antigen), IGF2 (insulin like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C—X—C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C—C motif) ligand 5), IRF1 (interferon regulatory factor 1), CFLAR (CASP8 and FADD-like apoptosis regulator), CALC A (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C—C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PEC AMI (platelet/endothelial cell adhesion molecule), CCL4 (chemokine (C—C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YMEl-like 1 (S. cerevisiae)), CAMP (cathelicidin antimicrobial peptide), ZC3II12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-b-methyltransf erase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C—C motif) ligand 11), PGF (placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHC1 (SHC (Src homology 2 domain containing) transforming protein 1), LRP1 (low density lipoprotein receptor-related protein 1), SOCS3 (suppressor of cytokine signaling 3), ADH1B (alcohol dehydrogenase IB (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor HI), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQOI (NAD (P) H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity IIa, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C—X—C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), F11 (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCF1E (butyrylcholinesterase), LIPE (lipase, hormone-sensitive), PRDX5 (peroxiredoxin 5), ADORA1 (adenosine A1 receptor), WRN (Werner syndrome, RecQ helicase-like), CXCR3 (chemokine (C—X—C motif) receptor 3), CD81 (CD81 molecule), SMAD7 (SMAD family member 7), LAMC2 (laminin, gamma 2), MAP3K5 (mitogen-activated protein kinase kinase kinase 5), CF1GA (chromogranin A (parathyroid secretory protein 1)), IAPP (islet amyloid polypeptide), RFIO (rhodopsin), ENPP1 (ectonucleotide pyrophosphatase/phosphodiesterase 1), PTF1LF1 (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISFI (cytokine inducible SF12-containing protein), GAST (gastrin), MYOC (myocilin, trabecular mesh work inducible glucocorticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), F1SF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTF1 (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOF1 (apolipoprotein FI (beta-2-glycoprotein I)), S100A8 (S100 calcium binding protein A8), IL11 (interleukin 11), ALOX15 (arachidonate 15-lipoxygenase), FBLN1 (fibulin 1), NR1F13 (nuclear receptor subfamily 1, group FI, member 3), SCD (stearoyl-CoA desaturase (delta-9-desaturase)), GIP (gastric inhibitory polypeptide), CF1 GB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), F1SD11B2 (hydroxy steroid (11-beta) dehydrogenase 2), CALCRL (calcitonin receptor-like), GALNT2 (UDP-N-acetyl-alpha-D-galactosamine: polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adeno virus E1B 19 kDa interacting protein 3), GCKR (glucokinase (hexokinase 4) regulator), S100A12 (S100 calcium binding protein A 12), PADI4 (peptidyl arginine deaminase, type IV), HSPA14 (heat shock 70 kDa protein 14), CXCR1 (chemokine (C—X—C motif) receptor 1), H19 (H19, imprinted maternally expressed transcript (non-protein coding)), KRTAP19-3 (keratin associated protein 19-3), insulin, RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNA1C (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11 A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol (myo)-1 (or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member B1), PARP2 (poly(ADP-ribose) polymerase 2), STAR (steroidogenic acute regulatory protein), LBP (lipopolysaccharide binding protein), ABCC6 (ATP-binding cassette, sub-family C (CFTR/MRP), member 6), RGS2 (regulator of G-protein signaling 2, 24 kDa), EFNB2 (ephrin-B2), cystic fibrosis transmembrane conductance regulator (CFTR), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C—C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (Fll receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), K′HK (ketohexokinase (fructokinase)), SAT1 (spermidine/spermine N1-acetyltransferase 1), GGFI (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2 A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RFIOB (ras homolog gene family, member B), LY96 (lymphocyte antigen 96), FOXO1 (forkhead box 01), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPR1 (nuclear protein, transcriptional regulator, 1), 15-Sep (15 kDa selenoprotein), CILP2 (cartilage intermediate layer protein 2), TERC (telomerase RNA component), GGT2 (gamma-glutamyltransf erase 2), MT-COI (mitochondrially encoded cytochrome c oxidase I), UOX (urate oxidase, pseudogene), a CRISPR/Cas effector polypeptide, an enzymatically active CRISPR/Cas effector polypeptide (e.g., is capable of cleaving a target nucleic acid) and a CRISPR/Cas effector polypeptide that is not enzymatically active (e.g., does not cleave a target nucleic acid, but retains binding to the target nucleic acid). In some cases, the donor DNA encodes a wild-type version of any of the foregoing polypeptides; i.e., the donor DNA can encode a “normal” version that does not include a mutation(s) that results in reduced function, lack of function, or pathogenesis.

In some cases, the donor DNA comprises a nucleotide sequence encoding a fluorescent polypeptide. Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, m PI urn (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, can be encoded.

In some cases, the donor DNA encodes an RNA, e.g., an siRNA, a microRNA, a short hairpin RNA (shRNA), an anti-sense RNA, a riboswitch, a ribozyme, an aptamer, a ribosomal RNA, a transfer RNA, and the like.

A donor DNA can include, in addition to a nucleotide sequence encoding one or more gene products (e.g., an RNA and/or a polypeptide), one or more transcriptional control elements, e.g., a promoter, an enhancer, and the like. In some cases, the transcriptional control element is inducible. In some cases, the promoter is reversible. In some cases, the transcriptional control element is constitutive. In some cases, the promoter is functional in a eukaryotic cell. In some cases, the promoter is a cell type-specific promoter. In some cases, the promoter is a tissue-specific promoter.

The nucleotide sequence of the donor DNA is typically not identical to the target nucleic acid (e.g., genomic sequence) that it replaces. Rather, the donor DNA may contain at least one or more single base changes, insertions, deletions, inversions or rearrangements with respect to the target nucleic acid (e.g., genomic sequence), so long as sufficient homology is present to support homology-directed repair (e.g., for gene correction, e.g., to convert a disease-causing base pair or a non-disease-causing base pair). In some cases, the donor DNA comprises a non-homologous sequence flanked by two regions of homology, such that homology-directed repair between the target DNA region and the two flanking sequences results in insertion of the non-homologous sequence at the target region. Donor DNA may also comprise a vector backbone containing sequences that are not homologous to the DNA region of interest (the target nucleic acid) and that are not intended for insertion into the DNA region of interest (the target nucleic acid). Generally, the homologous region(s) of a donor sequence will have at least 50% sequence identity to a target nucleic acid (e.g., a genomic sequence) with which recombination is desired. In certain cases, 60%, 70%, 80%, 90%, 95%, 98%, 99%, or 99.9% sequence identity is present. Any value between 1% and 100% sequence identity can be present, depending upon the length of the donor polynucleotide.

The donor DNA may comprise certain nucleotide sequence differences as compared to the target nucleic acid (e.g., genomic sequence), where such difference include, e.g. restriction sites, nucleotide polymorphisms, selectable markers (e.g., drug resistance genes, fluorescent proteins, enzymes etc.), etc., which may be used to assess for successful insertion of the donor DNA at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence. In some cases, the donor DNA will include one or more nucleotide sequences to aid in localization of the donor to the nucleus of the recipient cell or to aid in the integration of the donor DNA into the target nucleic acid. For example, in some case, the donor DNA may comprise one or more nucleotide sequences encoding one or more nuclear localization signals (e.g. PKKKRKV, VSRKRPRP, QRKRKQ, and the like (Frietas et al (2009) Cun-Genomics 10:550-7). In some cases, the donor DNA will include nucleotide sequences to recruit DNA repair enzymes to increase insertion efficiency. Fiuman enzymes involved in homology directed repair include MRN-CtIP, BLM-DNA2, Exo1, ERCC1, Rad51, Rad52, Ligase 1, RoIQ, PARP1, Ligase 3, BRCA2, RecQ/BLM-ToroIIIa, RTEL, Roïd, and Roth (Verma and Greenburg (2016) Genes Dev. 30 (10): 1138-1154). In some cases, the donor DNA is delivered as reconstituted chromatin (Cruz-Becerra and Kadonaga (2020) eLife 2020; 9: e55780 DOI: 10.7554/eLife.55780).

In some cases, the ends of the donor DNA are protected (e.g., from exonucleolytic degradation) by any convenient method and such methods are known to those of skill in the art. For example, one or more dideoxynucleotide residues can be added to the 3′ terminus of a linear molecule and/or self complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor DNA, additional lengths of sequence may be included outside of the regions of homology that can be degraded without impacting recombination.

### Linkers and Cleavable Peptides

In some embodiments, the gene editing systems may comprise two or more polypeptides that are coupled together by a linker. For example, a nuclease may be coupled or fused to an accessory protein by a linker. Such accessory functions can include deaminases, nucleases, reverse transcriptases, and recombinases. One or more gRNAs directed to such promoters or enhancers may also be provided to direct the binding of the nucleic acid programmable nuclease to such promoters or enhancers. The term linker as used in reference to a fusion protein refers to a molecule which joins the proteins to form a fusion protein. Generally, such molecules have no specific biological activity other than to join or to preserve some minimum distance or other spatial relationship between the proteins. However, in one embodiment, the linker may be selected to influence some property of the linker and/or the fusion protein such as the folding, net charge, or hydrophobicity of the linker.

In some embodiments, the mRNA payloads of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLOS ONE 6: e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one nucleobase editing system component/polypeptide separately within the same molecule) may be suitable for use as provided herein.

Suitable linkers for use in the methods of the present disclosure are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate a nuclease polypeptide and an accessory protein (e.g., a nucleotide deaminase) by a distance sufficient to ensure that each protein retains its required functional property. Preferred peptide linker sequences adopt a flexible extended conformation and do not exhibit a propensity for developing an ordered secondary structure. In one embodiment, the linker can be a chemical moiety which can be monomeric, dimeric, multimeric or polymeric. Preferably, the linker comprises amino acids. Typical amino acids in flexible linkers include Gly, Asn and Ser.

Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40:39-46; Murphy et al. (1986) Proc. Nat'l. Acad. Sci. USA 83:8258-62; U.S. Pat. Nos. 4,935,233; and 4,751,180. For example, GlySer linkers may be based on repeating units of GGS, i.e., up to 2, 3, 4, 5, 6, 7, 8, 9, 10, 11 or even 12 or more repeating units. In another example, GlySer linkers may be based on repeating units of GSG, i.e., up to 2, 3, 4, 5, 6, 7, 8, 9, 10, 11 or even 12 or more repeating units. In yet another example, GlySer linkers may be based on repeating units of GGGS, i.e., up to 2, 3, 4, 5, 6, 7, 8, 9, 10, 11 or even 12 or more repeating units. In still another example, GlySer linkers may be based on repeating units of GGGGS, i.e., up to 2, 3, 4, 5, 6, 7, 8, 9, 10, 11 or even 12 or more repeating units.

In yet a further embodiment, LEPGEKPYKCPECGKSFSQSGALTRHQRTHTR (SEQ ID NO: 33) is used as a linker.

In yet an additional embodiment, the linker is an XTEN linker, which is TCGGGATCTGAGACGCCTGGGACCTCGGAATCGGCTACGCCCGAAAGT (SEQ ID NO: 34). In particular embodiments, the Cas12a polypeptide is linked to the deaminase protein or its catalytic domain by means of an LEPGEKPYKCPECGKSFSQSGALTRHQRTHTRLEPGEKPYKCPECGKSFSQSGALTRHQRTHT R (SEQ ID NO: 35) linker. In further particular embodiments, Cas12a polypeptide is linked C-terminally to the N-terminus of a deaminase protein or its catalytic domain by means of an LEPGEKPYKCPECGKSFSQSGALTRHQRTHTRLEPGEKPYKCPECGKSFSQSGALTRHQRTHT RLEPGEKPYKCPECGKSFSQSGALTRHQRTHTR (SEQ ID NO: 36) linker. In addition, N- and C-terminal NLSs can also function as linker (e.g., PKKKRKVEASSPKKRKVEAS (SEQ ID NO: 37)).

The above description of linkers is intended to be non-limiting and includes any combinations of the above linkers or heterologous combinations of repeating GlySer linkers.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminoHEXAnoic acid (Ahx). In certain embodiments, the linker is based on a carbocyclic moiety (e.g., cyclopentane, cycloHEXAne). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLOS ONE 6: e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure.

Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one nucleobase editing system component/polypeptide separately within the same molecule) may be suitable for use as provided herein.

### Nuclear Localization Domains

In various embodiments, the gene editing systems or any of the components thereof may fused to one or more nuclear localization sequences (NLSs), such as about or more than about 1, 2, 3, 4, 5, 6, 7, 8, 9, 10, or more NLSs. In one embodiment, a gene editor component (e.g., a nucleic acid programmable DNA binding protein or an editing accessory protein) comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In an embodiment of the present disclosure, an editor component polypeptide comprises at most 6 NLSs. In one embodiment, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Nonlimiting examples of NLSs include an NLS sequence derived from: the NLS of the SV40 virus large T-antigen, having the amino acid sequence PKKKRKV, the NLS from nucleoplasmin (e.g. the nucleoplasmin bipartite NLS with the sequence KRPAATKKAGQAKKKK (SEQ ID NO: 38); the c-myc NLS having the amino acid sequence PAAKRVKLD or RQRRNELKRSP (SEQ ID NO: 39); the hRNPA1 M9 NLS having the sequence NQSSNFGPMKGGNFGGRSSGPYGGGGQYFAKPRNQGGY (SEQ ID NO: 40); the sequence RMRIZFKNKGKDTAELRRRRVEVSVELRKAKKDEQILKRRNV (SEQ ID NO: 41) of the IBB domain from importin-alpha; the sequences VSRKRPRP and PPKKARED of the myoma T protein; the sequence PQPKKKPL of human p53; the sequence SALIKKKKKMAP (SEQ ID NO: 42) of mouse c-abl IV; the sequences DRLRR and PKQKKRK of the influenza virus NS 1; the sequence RKLKKKIKKL (SEQ ID NO: 43) of the Hepatitis virus delta antigen; the sequence REKKKFLKRR (SEQ ID NO: 44) of the mouse Mxl protein; the sequence KRKGDEVDGVDEVAKKKSKK (SEQ ID NO: 45) of the human poly(ADP-ribose) polymerase; and the sequence RKCLQAGMNLEARKTKK (SEQ ID NO: 46) of the steroid hormone receptors (human) glucocorticoid.

In general, the one or more NLSs are of sufficient strength to drive accumulation of a gene editing component (e.g., a nuclease polypeptide) in a detectable amount in the nucleus of a eukaryotic cell. In general, strength of nuclear localization activity may derive from the number of NLSs in the NLS-modified polypeptide, the particular NLS(s) used, or a combination of these factors. Detection of accumulation in the nucleus may be performed by any suitable technique.

For example, a detectable marker may be fused to a gene editing component polypeptide, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of complex formation (e.g., assay for DNA cleavage or mutation at the target sequence, or assay for altered gene expression activity affected by complex formation and/or Cas 12a polypeptide activity), as compared to a control no exposed to the nuclease polypeptide or complex, or exposed to a nuclease polypeptide lacking the one or more NLSs.

In one embodiment of the present disclosure, at least one nuclear localization signal (NLS) is attached to the nucleic acid sequences encoding a component of a gene editing system described herein. In preferred embodiments at least one or more C-terminal or N-terminal NLSs are attached (and hence nucleic acid molecule(s) coding for an NLS-modified polypeptide can include coding for NLS(s) so that the expressed product has the NLS(s) attached or connected). In a preferred embodiment a C-terminal NLS is attached for optimal expression and nuclear targeting in eukaryotic cells, preferably human cells. The present disclosure also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

In other examples, the one or more nuclear localization signals is selected or derived from SV40, c-Myc or NLP-1.

The NLS examples above are non-limiting. The proteins contemplated herein may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

### Tag Domains

In some embodiments, the herein disclosed editing systems or components thereof may comprise a polypeptide tag, such as an affinity tag (chitin binding protein (CBP), maltose binding protein (MBP), glutathione-S-transferase (GST), SBP-tag, Strep-tag, AviTag, Calmodulin-tag); solubilization tag; chromatography tag (polyanionic amino acid tag, such as FLAG-tag); epitope tag (short peptide sequences that bind to high-affinity antibodies, such as V5-tag, Myc-tag, VSV-tag, Xpress tag, E-tag, S-tag, and HA-tag); fluorescence tag (e.g., GFP). In some embodiments, an editing system peptide may comprise an amino acid tag, such as one or more lysines, histidines, or glutamates, which can be added to the polypeptide sequences (e.g., at the N-terminal or C-terminal ends). Lysines can be used to increase peptide solubility or to allow for biotinylation. Protein and amino acid tags are peptide sequences genetically grafted onto a recombinant protein. Sequence tags are attached to proteins for various purposes, such as peptide purification, identification, or localization, for use in various applications including, for example, affinity purification, protein array, western blotting, immunofluorescence, and immunoprecipitation. Such tags are subsequently removable by chemical agents or by enzymatic means, such as by specific proteolysis or intein splicing.

Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

### Aptamers

In particular embodiments, the nucleic acid components (e.g., guide RNA) of the herein disclosed editing systems may further comprise a functional structure designed to improve nucleic acid component molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Sarnie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, a gene editing nucleic acid component is modified, e.g., by one or more aptamer(s) designed to improve RNA or DNA component molecule delivery, including delivery across the cellular membrane, to intracellular compartments, or into the nucleus. Such a structure can include, either in addition to the one or more aptamer(s) or without such one or more aptamer(s), moiety(ies) so as to render the nucleic acid component molecule deliverable, inducible or responsive to a selected effector. The present disclosure accordingly comprehends a reRNA component molecule that responds to normal or pathological physiological conditions, including without limitation pH, hypoxia, oxygen concentration, temperature, protein concentration, enzymatic concentration, lipid structure, light exposure, mechanical disruption (e.g. ultrasound waves), magnetic fields, electric fields, or electromagnetic radiation.

Agents that Modulate DNA-Repair

In certain embodiments, the gene editing systems described herein (e.g., an engineered nucleic acid construct or engineered nucleic acid-enzyme construct described herein) further comprises or encodes a DNA-repair modulating biomolecule, which may further enhance the efficiency of integration of a transgene on the heterologous nucleic acid by homology dependent repair (HDR).

In certain embodiments, the DNA-repair modulating biomolecule comprises a Nonhomologous end joining (NHEJ) inhibitor.

In certain embodiments, the DNA-repair modulating biomolecule comprises a homologous directed repair (HDR) promoter.

In certain embodiments, the DNA-repair modulating biomolecule comprises a NHEJ inhibitor and an HDR promoter.

In certain embodiments, the DNA-repair modulating biomolecule enhances or improves more precise genome editing and/or the efficiency of homologous recombination, compared to the otherwise identical embodiment without the DNA-repair modulating biomolecule.

HDR promoters and/or NHEJ inhibitors can, in some embodiments, comprise one or more small molecules. Systems bearing recombination enhancers such as small molecules that activate HDR and suppress NHEJ locally at the genomic site of the DNA damage can be tailored in their placement on the engineered systems to further enhance their efficiency. In general, the small molecule recombination enhancers can be synthesized to bear linkers and a functional group, such as maleimide for reacting with a thiol group on a Cys residue of a protein, for chemical conjugation to the engineered systems. Use of commercially available functionalized PEG linkers (alkyne, azide, cyclooctyne etc.) can also be employed for conjugation, and orthogonal conjugation chemistries can be utilized for the multivalent display.

Conjugation sites can be readily identified where modifications do not affect the potency of the recombination enhancers selected.

In certain embodiments, multivalent display of one or more DNA-repair modulating biomolecule can be affected, including multiple moieties of NHEJ inhibitors, HDR promoters, or a combination thereof. See, for example, “Genomic targeting of epigenetic probes using a chemically tailored Cas9 system” by Liszczak et al., Proc Natl Acad Sci U.S.A. 114:681-686, 2017 (incorporated herein by reference). In certain embodiments, multivalent display of small molecule compounds can be achieved through sortase loop proteins used as a scaffold for their display.

In some embodiments, the DNA-repair modulating biomolecule may comprise an HDR promoter. The HDR promoter may comprise small molecules, such as RSI or analogs thereof. In certain embodiments, the HDR promoter stimulates RAD51 activity or RAD52 motif protein 1 (RDM1) activity. In certain embodiments, the HDR promoter comprises Nocodazole, which can result in higher HDR selection.

In certain embodiments, the HDR promoter may be administered prior to the delivery of the engineered systems described herein.

In certain embodiments, the HDR promoter locally enhances HDR without NHEJ inhibition. For example, RAD51 is a protein involved in strand exchange and the search for homology regions during HDR repair. In certain embodiments, the HDR promoter is phenylbenzamide RSI, identified as a small-molecule RAD51-stimulator (see WO2019/135816 at [0200]-[0204], specifically incorporated herein by reference).

In certain embodiments, the DNA-repair modulating biomolecule comprises C-terminal binding protein interacting protein (CtIP) or a functional fragment or homolog thereof. CtIP is a key protein in early steps of homologous recombination. According to this embodiment, the CtIP or the functional fragment or homolog thereof can be linked (e.g., fused) to the RT or the sequence-specific nuclease (e.g., a CRISPR/Cas effector enzyme, a ZFN, a TALEN, a meganuclease, TnpB, IscB, or a restriction endonuclease (RE)), and stimulates transgene integration by HDR.

In certain embodiments, the CtIP fragment is a minimal N-terminal fragment of the wild-type CtIP, such as the N-terminal fragment comprising residues 1-296 of the full-length CtIP (the HE for HDR enhancer), as described in Charpentier et al. (Nature Comm., DOI: 10.1038/s41467-018-03475-7, incorporated herein by reference), shown to be sufficient to stimulate HDR. The activity of the fragment depends on CDK phosphorylation sites (e.g., S233, T245, and S276) and the multimerization domain essential for CtIP activity in homologous recombination. Thus alternative fragments comprising the CDK phosphorylation sites and the multimerization domain essential for CtIP activity are also within the scope of the present disclosure.

In certain embodiments, the DNA-repair modulating biomolecule comprises a dominant negative 53BP1.

In certain embodiments, the DNA-repair modulating biomolecule comprises a cell cycle-specific degradation tag, such as the degradation domain of the (human) Geminin, and the (murine) CyclinB2.

In certain embodiments, the DNA-repair modulating biomolecule comprises CyclinB2, a member of the B-type cyclins that associate with p34cdc2, and an essential component of the cell cycle regulatory machinery. CRISPR-mediated knock-in efficiency may be increased by promoting the relative increase in Cas9 activity in G2 phase of the cell cycle, when HDR is more active. In certain embodiments, the degradation domains of the (human) Geminin and (murine) CyclinB2 can be used as either N- or C-terminal fusion to serve as the DNA-repair modulating biomolecule. These domains are known to determine a cell-cycle specific profile of chimeric proteins, namely an increase in their relative concentration in S and G2 compared to G1, high-jacking the conventional CyclinB2 and Geminin degradation pathways. This produces active Geminin-Cas9 and CyclinB2-Cas9 chimeric proteins, which are degraded in a cell-cycle-dependent manner. Such chimeras shift the repair of the DSBs to the HDR repair pathway compared to the commonly used Cas9.

While not wishing to be bound by particular theory, it is believed that the application of such cell cycle-specific degradation tags permits/promotes more efficient/secure gene editing.

In certain embodiments, the DNA-repair modulating biomolecule comprises a Rad family member protein, such as Rad50, Rad51, Rad52, etc., which functions to promote foreign DNA integration into a host chromosome. Specifically, Rad52 is an important homologous recombinant protein, and its complex with Rad51 plays a key role in HDR, mainly involved in the regulation of foreign DNA in eukaryotes. Key steps in the process of HR include repair mediated by Rad51 and strand exchange. Co-expression of Rad52 as a DNA-repair modulating biomolecule significantly enhances the likelihood of HDR by, e.g., three-fold.

In certain embodiments, the DNA-repair modulating biomolecule comprises a RAD52 protein as, e.g., either an N- or a C-terminal fusion.

In certain embodiments, the DNA-repair modulating biomolecule comprises a RAD52 motif protein 1 (RDM1) that functions similarly as RAD52. RDM1 has been shown to be able to repair DSBs caused by DNA replication, prevent G2 or M cell cycle arrest, and improve HDR selection.

In certain embodiments, the DNA-repair modulating biomolecule comprises a dominant negative version of the tumor suppressor p53-binding protein 1 (53BP1). The wild-type protein 53BP1 is a key regulator of the choice between NHEJ and HDR—it is a pro-NHEJ factor which limits HDR by blocking DNA end resection, and also by inhibiting BRCA1 recruitment to DSB sites. It has been shown that global inhibition of 53BP1 by a ubiquitin variant significantly improves Cas9-mediated HDR frequency in non-hematopoietic and hematopoietic cells with single-strand oligonucleotide delivery or double-strand donor in AAV.

In certain embodiments, the dominant negative (DN) version of the 53BP1 comprises the minimal focus forming region, but lacks domains outside this region, e.g., towards the N-terminus and tandem C-terminal BRCT repeats that recruit key effectors involved in NHEJ, such as RIFI-PTIP and EXPAND, respectively. The 53BP1 adapter protein is recruited to specific histone marks at sites of DSBs via this minimal focus forming region, which comprises several conserved domains including an oligomerization domain (OD), a glycine-arginine rich (GAR) motif, a Tudor domain, and an adjacent ubiquitin-dependent recruitment (UDR) motif. The Tudor domain mediates interactions with histone H4 dimethylated at K2023.

In certain embodiments, a dominant negative version of 53BP1 (DN1S) suppresses the accumulation of endogenous 53BP1 and downstream NHEJ proteins at sites of DNA damage, while upregulating the recruitment of the BRCA1 HDR protein. Such a DN version of the 53BP1 can be used as the DNA-repair modulating biomolecule, either as an N- or a C-terminal fusion (such as a Cas9 fusion, to locally inhibit NHEJ at the Cas9-target site defined by its gRNA, while promoting an increase in HDR, and does not globally affect NHEJ, thereby improving cell viability).

In certain embodiments, the DNA-repair modulating biomolecule comprises an NHEJ inhibitor, such as an inhibitor of DNA ligase IV, a KU inhibitor (e.g., KU70 or KU80), a DNA-PKc inhibitor, or an artemis inhibitor.

In certain embodiments, the NHEJ inhibitor inhibits the NHEJ pathway, enhances HDR, or modulates both. In certain embodiments, the NHEJ inhibitor is a small molecule inhibitor.

In certain embodiments, the small molecule inhibitor of the NHEJ pathway comprises an SCR7 analog, for example, PK66, PK76, PK409.

In certain embodiments, the NHEJ inhibitor comprises a KU inhibitor, for example, KU5788, and KU0060648.

In certain embodiments, a small molecule NHEJ inhibitor is linked to a polyglycine tripeptide through PEG for sortase-mediated ligation, as described in WO2019/135816, Guimaraes et al., Nat Protoc 8:1787-99, 2013; Theile et al., Nat Protoc 8:1800-7, 2013; and Schmohl et al., Curr Opin Chem Biol 22:122-8, 2014 (all incorporated herein by reference). The same means can also be used for attaching small molecule HDR enhancers to protein.

An exemplary method for conjugating a small molecule DNA-repair modulating biomolecule without loss of activity is described in WO2019135816, where SCR-7 conjugation of a poly-glycine peptide with the para-carboxylic moiety at ring 4 retained activity of the inhibitor, with rings 1, 2 and 3 of the molecule having involvement in the target-engagement, providing a simple and effective strategy to ligate a small molecule NHEJ inhibitor to the system described herein (e.g., to the sequence-specific nuclease including Cas enzymes, or to the RT) to precisely enhance HDR pathway near a nucleic acid target site.

In certain embodiments, a nucleic acid targeting moiety conjugates based on small molecule inhibitor of DNA-dependent protein kinase (DNA-PK) or heterodimeric Ku (KU70/KU80) can be utilized. KU-0060648 is one potent KU-inhibitors, which can also be functionalized with poly-glycine and used for recombination enhancement.

In certain embodiments, the DNA-repair modulating biomolecule comprises the Tumor Suppressor p53. p53 plays a direct role in DNA repair, including HR regulation, where it affects the extension of new DNA, thereby affecting HDR selection. In vivo, p53 binds to the nuclear matrix and is a rate-limiting factor in repairing DNA structure. p53 regulates DNA repair processes in almost all eukaryotes via transactivation-dependent and -independent pathways, but only the transactivation-independent function of p53 is involved in HR regulation. Wild-type p53 protein can link double stranded breaks to form intact DNA, as well as also playing a role in inhibiting NHEJ. p53 interacts with HR-related proteins, including Rad51, where it controls HR through direct interaction with Rad51.

### Accessory Domains

In other aspects, the gene editing systems may comprise one or more additional accessory proteins having genome modifying functions, including recombinases, invertases, nucleases, polymerases, ligases, deaminases, reverse transcriptases, or epigenetic modifying functions. In various embodiments, the accessory proteins may be provided separately. In other embodiments, the accessory proteins may be fused to Cas12a, optionally with a linker.

The gene editing systems may further comprise additional polypeptides polypeptides, proteins and/or peptides known in the art. Non-limiting categories of polypeptides include antigens, antibodies, antibody fragments, cytokines, peptides, hormones, enzymes, oxidants, antioxidants, synthetic polypeptides, and chimeric polypeptides, receptor, enzymes, hormones, transcription factors, ligands, membrane transporters, structural proteins, nucleases, or a component, variant or fragment (e.g., a biologically active fragment) thereof.

As used herein, the term “peptide” generally refers to shorter polypeptides of about 50 amino acids or less. Peptides with only two amino acids may be referred to as “dipeptides.” Peptides with only three amino acids may be referred to as “tripeptides.” Polypeptides generally refer to polypeptides with from about 4 to about 50 amino acids. Peptides may be obtained via any method known to those skilled in the art. In some embodiments, peptides may be expressed in culture. In some embodiments, peptides may be obtained via chemical synthesis (e.g., solid phase peptide synthesis).

In some embodiments, the RNA payloads (e.g., linear and/or circular mRNA payloads encoding one or more encoded products of interest or the non-coding RNAs such as guide RNAs) may encode a user-programmable DNA binding protein, or a gene editor accessory proteins, such as, but not limited to a deaminases, nucleases, transposases, polymerases, and reverse transcriptases, etc.

In some embodiments, the RNA payloads (e.g., linear and/or circular mRNA payloads encoding one or more encoded products of interest), e.g., the originator constructs and benchmark constructs described herein, may encode a simple protein associated with a non-protein. Non-limiting examples of conjugated proteins include, glycoproteins, hemoglobins, lecithoproteins, nucleoproteins, and phosphoproteins.

In some embodiments, the RNA payloads (e.g., linear and/or circular mRNA payloads encoding one or more encoded products of interest), e.g., the originator constructs and benchmark constructs described herein, may encode a protein that is derived from a simple or conjugated protein by chemical or physical means. Non-limiting examples of derived proteins include denatured proteins and peptides.

In some embodiments, the polypeptide, protein or peptide may be unmodified.

In some embodiments, the polypeptide, protein or peptide may be modified. Types of modifications include, but are not limited to, phosphorylation, glycosylation, acetylation, ubiquitylation/sumoylation, methylation, palmitoylation, quinone, amidation, myristoylation, pyrrolidone carboxylic acid, hydroxylation, phosphopantetheine, prenylation, GPI anchoring, oxidation, ADP-ribosylation, sulfation, S-nitrosylation, citrullination, nitration, gamma-carboxyglutamic acid, formylation, hypusine, topaquinone (TPQ), bromination, lysine topaquinone (LTQ), tryptophan tryptophylquinone (TTQ), iodination, and cysteine tryptophylquinone (CTQ). In some aspects, the polypeptide, protein or peptide may be modified by a post-transcriptional modification which can affect its structure, subcellular localization, and/or function.

In some embodiments, the polypeptide, protein or peptide may be modified using phosphorylation. Phosphorylation, or the addition of a phosphate group to serine, threonine, or tyrosine residues, is one of most common forms of protein modification. Protein phosphorylation plays an important role in fine tuning the signal in the intracellular signaling cascades.

In some embodiments, the polypeptide, protein or peptide may be modified using ubiquitination which is the covalent attachment of ubiquitin to target proteins. Ubiquitination-mediated protein turnover has been shown to play a role in driving the cell cycle as well as in protein-degradation-independent intracellular signaling pathways.

In some embodiments, the polypeptide, protein or peptide may be modified using acetylation and methylation which can play a role in regulating gene expression. As a non-limiting example, the acetylation and methylation could mediate the formation of chromatin domains (e.g., euchromatin and heterochromatin) which could have an impact on mediating gene silencing.

In some embodiments, the polypeptide, protein or peptide may be modified using glycosylation. Glycosylation is the attachment of one of a large number of glycan groups and is a modification that occurs in about half of all proteins and plays a role in biological processes including, but not limited to, embryonic development, cell division, and regulation of protein structure. The two main types of protein glycosylation are N-glycosylation and O-glycosylation. For N-glycosylation the glycan is attached to an asparagine and for O-glycosylation the glycan is attached to a serine or threonine.

In some embodiments, the polypeptide, protein or peptide may be modified using sumoylation. Sumoylation is the addition of SUMOs (small ubiquitin-like modifiers) to proteins and is a post-translational modification similar to ubiquitination.

In other embodiments, the RNA payloads (e.g., linear and/or circular mRNA payloads encoding one or more encoded products of interest), e.g., the originator constructs and benchmark constructs described herein, may encode a therapeutic protein, such as those exemplified below.

In other embodiments, the RNA payloads (e.g., linear and/or circular mRNA payloads encoding one or more products of interest), e.g., the originator constructs and benchmark constructs described herein, may encode a gene editing system, such as those exemplified herein. As used herein, a “nucleobase editing system” is a protein, DNA, or RNA composition capable of making edits, modifications or alterations to one or more targeted genes of interest. According to the present disclosure, one or more nucleobase editing system currently being marketed or in development may be encoded by the RNA payloads (e.g., linear and/or circular mRNA payloads encoding one or more encoded products of interest) described herein of the present disclosure.

### Inducibility Modifications

In one embodiment, a gene editing system or component thereof may be inducible. The inducible nature of a system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the TnpB polypeptide may be a part of a Light Inducible Transcriptional Effector (LITE) to direct changes in transcriptional activity in a sequence-specific manner. The components of a light may include a TnpB polypeptide, a light-responsive cytochrome heterodimer (e.g. from Arabidopsis thaliana), and a transcriptional activation/repression domain. Further examples of inducible DNA binding proteins and methods for their use are provided in U.S. Provisional Application Nos. 61/736,465 and U.S. 61/721,283, and International Patent Publication No. WO 2014/018423 A2 which is hereby incorporated by reference in its entirety.

Once all copies of a gene in the genome of a cell have been edited, continued expression of the system in that cell is no longer necessary. Indeed, sustained expression would be undesirable in case of off-target effects at unintended genomic sites, etc. Thus time-limited expression would be useful. Inducible expression offers one approach, but in addition Applicants have engineered a self-inactivating system that relies on the use of a non-coding nucleic acid component molecule target sequence within the vector itself. Thus, after expression begins, the system will lead to its own destruction, but before destruction is complete it will have time to edit the genomic copies of the target gene (which, with a normal point mutation in a diploid cell, requires at most two edits). Simply, the self-inactivating system includes additional RNA (e.g., nucleic acid component molecule) that targets the coding sequence for the Cas12a polypeptide itself or that targets one or more non-coding nucleic acid component molecule target sequences complementary to unique sequences present in one or more of the following: (a) within the promoter driving expression of the non-coding RNA elements, (b) within the promoter driving expression of the Cas12a polypeptide gene, (c) within 100 bp of the ATG translational start codon in the Cas12a polypeptide coding sequence, (d) within the inverted terminal repeat (iTR) of a viral delivery vector, e.g., in the AAV genome.

In some aspects, a single nucleic acid component molecule is provided that is capable of hybridization to a sequence downstream of a nuclease polypeptide start codon, whereby after a period of time there is a loss of the nuclease polypeptide expression. In some aspects, one or more nucleic acid component molecule(s) are provided that are capable of hybridization to one or more coding or non-coding regions of the polynucleotide encoding the system, whereby after a period of time there is a inactivation of one or more, or in some cases all, of the system. In some aspects of the system, and not to be limited by theory, the cell may comprise a plurality of complexes, wherein a first subset of complexes comprise a first nucleic acid component molecule capable of targeting a genomic locus or loci to be edited, and a second subset of complexes comprise at least one second nucleic acid component molecule capable of targeting the polynucleotide encoding the system, wherein the first subset of complexes mediate editing of the targeted genomic locus or loci and the second subset of complexes eventually inactivate the system, thereby inactivating further expression in the cell.

The various coding sequences (e.g., a Cas12a polypeptide and nucleic acid component molecule) can be included on a single vector or on multiple vectors. For instance, it is possible to encode the enzyme on one vector and the various RNA sequences on another vector, or to encode the enzyme and one nucleic acid component molecule on one vector, and the remaining nucleic acid component molecule on another vector, or any other permutation. In general, a system using a total of one or two different vectors is preferred.

### IV. LNP Pharmaceutical Compositions

The LNP-based nucleobase editing systems, RNA therapeutics and pharmaceutical compositions thereof described herein can be formulate using one or more excipients to: (1) increase stability; (2) increase cell transfection or transduction; (3) permit the sustained or delayed expression of the payload; (4) alter the biodistribution (e.g., target the nucleobase editing systems to specific tissues or cell types); (5) increase the translation of encoded protein; (6) alter the release profile of encoded protein; and/or (7) allow for regulatable expression of an RNA payload expression product.

Formulations can include, without limitation, saline, liposomes, lipid nanoparticles, polymers, peptides, proteins, cells transfected with viral vectors (e.g., for transfer or transplantation into a subject) and combinations thereof.

Formulations of the LNP-based nucleobase editing systems, RNA therapeutics and pharmaceutical compositions thereof described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient (e.g., encapsulated LNP with an mRNA payload expressing a protein of interest) with an excipient and/or one or more other accessory ingredients. As used herein, the phrase “active ingredient” can refer to an LNP encapsulated with a payload mRNA, as well as to the mRNA payload construct itself, including originator constructs and benchmark construct as described herein.

Formulations of the encapsulated LNPs, the payload mRNA constructs, and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopocia (USP), the European Pharmacopocia (EP), the British Pharmacopoeia, and/or the International Pharmacopoeia.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, formulations described herein may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

In one embodiment, the formulations described herein comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl.Alpha.-Ionone; Almond Oil; Alpha-Terpineol; Alpha-Tocopherol; Alpha-Tocopherol Acetate, Dl-; Alpha-Tocopherol, Dl-; Aluminum Acetate; Aluminum Chlorhydroxy Allantoinate; Aluminum Hydroxide; Aluminum Hydroxide-Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Dicthanolamide; Coco Monocthanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, Dl-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, Dl-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Olcate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor Fig 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O FI-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, Dl-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa·S); Hypromellose 2910 (15000 Mpa·S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol-Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystalline Wax; Mineral Oil; Mono and Diglyceride; Monostearyl Citrate; Monothioglycerol; Multisterol Extract; Myristyl Alcohol; Myristyl Lactate; Myristyl-. Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90 g; Phosphoric Acid; Pine Needle Oil (Pinus sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy) Propane Anhydride): Sebacic Acid; Poly(Dimethylsiloxane/Methylvinylsiloxane/Methylhydrogensiloxane) Dimethylvinyl Or Dimethylhydroxy Or Trimethyl Endblocked; Poly(Dl-Lactic-Co-Glycolic Acid), (50:50; Poly(Dl-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether.Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, Dl-; Tenox; Tenox-2; Tert-Butyl Alcohol; Tert-Butyl Hydroperoxide; Tert-Butylhydroquinone; Tetrakis(2-Methoxyisobutylisocyanide) Copper (I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

In some embodiments, formulations disclosed herein may include cations or anions. The formulations include metal cations such as, but not limited to, Zn²⁺, Ca²⁺, Cu²⁺, Mn²⁺, Mg²⁺, and combinations thereof. As a non-limiting example, formulations may include polymers and complexes with a metal cation.

Formulations of the disclosure may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

A nanoparticle composition may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro: Lippincott, Williams & Wilkins, Baltimore, Md., 2006).

### V. Routes of Administration

The LNP-based nucleobase editing systems, RNA therapeutics and pharmaceutical compositions thereof described herein may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions may be administered in a way which allows them to cross the blood-brain barrier, vascular barrier, or other epithelial barrier. The originator constructs, benchmark constructs, and targeting systems may be administered in any suitable form, either as a liquid solution or suspension, as a solid form suitable for liquid solution or suspension in a liquid solution. The originator constructs, benchmark constructs, and targeting systems may be formulated with any appropriate and pharmaceutically acceptable excipient.

In some embodiments, the originator constructs, benchmark constructs, and targeting systems may be delivered to a subject via a single route administration.

In some embodiments, the originator constructs, benchmark constructs, and targeting systems may be delivered to a subject via a multi-site route of administration. A subject may be administered at 2, 3, 4, 5, or more than 5 sites.

In some embodiments, a subject may be administered the originator constructs, benchmark constructs, and targeting systems using a bolus infusion.

In some embodiments, a subject may be administered originator constructs, benchmark constructs, and targeting systems using sustained delivery over a period of minutes, hours, or days. The infusion rate may be changed depending on the subject, distribution, formulation or another delivery parameter.

In some embodiments, the originator constructs, benchmark constructs, and targeting systems may be delivered by intramuscular delivery route. Non-limiting examples of intramuscular administration include an intravenous injection or a subcutaneous injection.

In some embodiments, the originator constructs, benchmark constructs, and targeting systems may be delivered by oral administration. Non-limiting examples of oral delivery include a digestive tract administration and a buccal administration.

In some embodiments, the originator constructs, benchmark constructs, and targeting systems may be delivered by intraocular delivery route. A non-limiting example of intraocular delivery include an intravitreal injection.

In some embodiments, the originator constructs, benchmark constructs, and targeting systems may be delivered by intranasal delivery route. Non-limiting examples of intranasal delivery include nasal drops or nasal sprays.

In some embodiments, the originator constructs, benchmark constructs, and targeting systems may be administered to a subject by peripheral injections. Non-limiting examples of peripheral injections include intraperitoneal, intramuscular, intravenous, conjunctival, or joint injection.

In some embodiments, the originator constructs, benchmark constructs, and targeting systems may be delivered by injection into the cerebrospinal fluid. Non-limiting examples of delivery to the cerebrospinal fluid include intrathecal and intracerebroventricular administration.

In some embodiments, the originator constructs, benchmark constructs, and targeting systems may be delivered by systemic delivery. As a non-limiting example, the systemic delivery may be by intravascular administration.

In some embodiments, the originator constructs, benchmark constructs, and targeting systems may be administered to a subject by intracranial delivery.

In some embodiments, the originator constructs, benchmark constructs, and targeting systems may be administered to a subject by intraparenchymal administration.

In some embodiments, the originator constructs, benchmark constructs, and targeting systems may be administered to a subject by intramuscular administration.

In some embodiments, the originator constructs, benchmark constructs, and targeting systems are administered to a subject and transduce muscle of a subject. As a non-limiting example, the originator constructs, benchmark constructs, and targeting systems are administered by intramuscular administration.

In some embodiments, the originator constructs, benchmark constructs, and targeting systems may be administered to a subject by intravenous administration.

In some embodiments, the originator constructs, benchmark constructs, and targeting systems may be administered to a subject by subcutaneous administration.

In some embodiments, the originator constructs, benchmark constructs, and targeting systems may be administered to a subject by topical administration.

In some embodiments, the originator constructs, benchmark constructs, and targeting systems may be delivered by more than one route of administration.

The originator constructs, benchmark constructs, and targeting systems described herein may be co-administered in conjunction with one or more originator constructs, benchmark constructs, targeting systems, or therapeutic agents or moieties.

In some embodiments, pharmaceutical compositions and/or formulations described herein may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

In some embodiments, pharmaceutical compositions and/or formulations described herein may be formulated for administration topically. The skin may be an ideal target site for delivery as it is readily accessible. Three routes are commonly considered to deliver pharmaceutical compositions and/or formulations described herein to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions).

In some embodiments, pharmaceutical compositions and/or formulations described herein may be delivered using a variety of dressings (e.g., wound dressings) or bandages (e.g., adhesive bandages) for conveniently and/or effectively carrying out methods described herein. Typically dressing or bandages may comprise sufficient amounts of pharmaceutical compositions and/or formulations described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, contemplated herein is the use of transdermal patches, which often have the added advantage of providing controlled delivery of pharmaceutical compositions and/or formulations described herein to the body. Such dosage forms may be prepared, for example, by dissolving and/or dispensing pharmaceutical compositions and/or formulations described herein in the proper medium. Alternatively, or additionally, rates may be controlled by either providing rate controlling membranes and/or by dispersing pharmaceutical compositions and/or formulations described herein in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

In some embodiments, pharmaceutical compositions and/or formulations described herein may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

In some embodiments, pharmaceutical compositions and/or formulations described herein may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

In some embodiments, pharmaceutical compositions and/or formulations described herein are formulated in depots for extended release.

In some embodiments, pharmaceutical compositions and/or formulations described herein are spatially retained within or proximal to target tissues. Provided are methods of providing pharmaceutical compositions and/or formulations described herein to target tissues of mammalian subjects by contacting target tissues (which comprise one or more target cells) with pharmaceutical compositions and/or formulations described herein under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions and/or formulations described herein that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of pharmaceutical compositions and/or formulations described herein administered to subjects are present intracellularly at a period of time following administration. For example, intramuscular injection to mammalian subjects may be performed using aqueous compositions comprising an active ingredient and one or more transfection reagents, and retention is determined by measuring the amount of active ingredient present in muscle cells.

In some embodiments, provided are methods for delivering pharmaceutical compositions and/or formulations described herein to target tissues of mammalian subjects, by contacting target tissues (comprising one or more target cells) with pharmaceutical compositions and/or formulations described herein under conditions such that they are substantially retained in such target tissues. Pharmaceutical compositions and/or formulations described herein comprise enough active ingredient such that the effect of interest is produced in at least one target cell. In some embodiments, pharmaceutical compositions and/or formulations described herein generally comprise one or more cell penetration agents, although “naked” formulations (such as without cell penetration agents or other agents) are also contemplated, with or without pharmaceutically acceptable carriers.

In some embodiments, pharmaceutical compositions and/or formulations described herein may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

In some embodiments, pharmaceutical compositions and/or formulations described herein may be administered nasally and/or intranasal. In some embodiments, formulations described herein useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

In some embodiments, pharmaceutical compositions and/or formulations described herein may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### VI. Methods of Use

The LNP-based compositions described herein may be used to deliver a nucleobase editing system to a cell or tissue of interest. In certain embodiments, the LNP-based compositions described herein are useful for executing one or more edits, modifications or alterations to one or more targeted genes of interest. In certain embodiments, the one or more edits, modifications or alterations to the one or more targeted genes of interest are capable of treating a disease or disorder in a patient in need thereof. In certain embodiments, the cells of interest are red blood cell progenitor cells, including hematopoietic stem cells. In certain embodiments, the disease or disorder to be treated is a hemoglobinopathy, such as sickle cell disease or beta-thalassemia.

### A. Methods of Producing Polypeptides in Cells

The present disclosure provides methods of producing a polypeptide of interest in a mammalian cell. Methods of producing polypeptides involve contacting a cell with an LNP comprising a nucleobase editing system comprising an mRNA encoding a component of said nucleobase editing system and/or a formulation or composition thereof as described herein. Upon contacting the cell with the lipid nanoparticle, the mRNA may be taken up and translated in the cell to produce a polypeptide of interest, e.g., an enzyme component of the nucleobase editing system.

In general, the step of contacting a mammalian cell with a LNP including an mRNA encoding a polypeptide of interest may be performed in vivo, ex vivo, in culture, or in vitro. In certain preferred embodiments, the step of contacting occurs in vivo. In certain alternative embodiments, the step of contacting occurs ex vivo. The amount of lipid nanoparticle contacted with a cell, and/or the amount of mRNA therein, may depend on the type of cell or tissue being contacted, the means of administration, the physiochemical characteristics of the lipid nanoparticle and the mRNA (e.g., size, charge, and chemical composition) therein, and other factors. In general, an effective amount of the lipid nanoparticle will allow for efficient polypeptide production in the cell. Metrics for efficiency may include polypeptide translation (indicated by polypeptide expression), level of mRNA degradation, and immune response indicators.

The step of contacting an LNP including an mRNA with a cell may involve or cause transfection. A phospholipid including in the lipid component of a LNP may facilitate transfection and/or increase transfection efficiency, for example, by interacting and/or fusing with a cellular or intracellular membrane. Transfection may allow for the translation of the mRNA within the cell.

In some embodiments, the lipid nanoparticles described herein is used therapeutically. For example, an mRNA included in an LNP may encode a polypeptide (e.g., in a translatable region) that enables editing, modifying, or altering a target polynucleotide sequence and therefore produces a therapeutic effect upon entry (e.g., transfection) into a cell.

### B. Therapeutic Methods Using LNPs Described Herein

Provided herein are therapeutic methods for treating a disease or disorder by using the LNP-based RNA compositions described herein to deliver one or more therapeutic gene editing systems to a cell, organ, or tissue. In preferential embodiments, delivery occurs in vivo in a living patient's body.

Delivery of a therapeutic and/or prophylactic to a cell involves administering a composition of the disclosure that comprises a LNP encapsulated with a payload, where administration of the composition involves contacting the cell with the composition. Upon contacting a cell with the lipid nanoparticle, a translatable mRNA may be translated in the cell to produce a polypeptide of interest. However, RNAs that are substantially not translatable may also be delivered to cells. Substantially non-translatable RNAs may be useful as components of nucleobase editing systems and/or may sequester translational components of a cell to reduce expression of other species in the cell.

In some embodiments, an LNP of the present disclosure may target a particular type or class of cells (e.g., cells of a particular organ or system thereof), preferentially or selectively delivering the payload to that particular type or class of cells. In some embodiments, a LNP including an RNA payload of interest is specifically delivered to red blood cell progenitor cells. “Specific delivery” to a particular class of cells, an organ, or a system or group thereof implies that a higher proportion of lipid nanoparticles including a therapeutic and/or prophylactic are delivered to the destination (e.g., tissue or cell) of interest relative to other destinations upon administration of an LNP to a mammal. In some embodiments, specific delivery may result in a greater than 2-fold, 5-fold, 10-fold, 15-fold, or 20-fold increase in the amount of therapeutic and/or prophylactic per 1 g of tissue of the targeted destination (e.g., tissue of interest, such as a bone marrow) as compared to another destination (e.g., the liver). In some embodiments, the cell type of interest is selected from the group consisting of erythroid progenitor cells, myeloid progenitor cells, hematopoietic stem cells (HSCs), and erythrocytes. In certain embodiments, the cell type of interest is long-term HSCs (LT-HSC). In certain embodiments, the cell type of interest is short-term HSCs (ST-HSC). In certain embodiments, the cell type of interest is multipotent progenitors (MPP). In certain embodiments, the cell type of interest is common myeloid progenitors (CMP). In certain embodiments, the cell type of interest is megakaryocyte erythroid progenitors (MEP).

Lipid nanoparticles described herein are useful for treating a disease, disorder, or condition. In particular, such compositions are useful in treating a disease, disorder, or condition characterized by missing or aberrant protein or polypeptide activity. In some embodiments, a formulation of the disclosure comprises an LNP including a gene editing system capable of correcting the genes encoding the aberrant protein is administered or delivered to a cell. In some embodiments, a formulation of the disclosure comprises an LNP including a gene editing system capable of activating genes encoding the missing protein is administered or delivered to a cell. Administration of gene editing system to a progenitor cell may lead to permanent correction of the aberrant protein and/or production of the missing protein, thereby reducing or eliminating an issue caused by the absence of or aberrant activity caused by the protein. Because translation of a payload and subsequent gene editing may occur rapidly (over the course of days or weeks), the methods and compositions may be useful in the treatment of acute diseases, disorders, or conditions. A therapeutic and/or prophylactic included in an LNP may also be capable of altering the rate of transcription of a given species, thereby affecting gene expression.

Diseases, disorders, and/or conditions characterized by dysfunctional or aberrant protein or polypeptide activity for which a composition may be administered include but are not limited to hemoglobinopathies. Multiple such diseases, disorders, and/or conditions may be characterized by missing or aberrant (or substantially diminished such that proper protein function does not occur) protein activity. Such proteins may not be present, or they may be essentially non-functional.

### C. Hemoglobinopathies

In various embodiments, the LNP-based therapeutics and pharmaceutical compositions thereof described herein may be used to treat hemoglobinopathies. In certain embodiments, the LNPs of the present disclosure can be used to treat sickle cell disease (SCD). In certain embodiments, the LNPs of the present disclosure can be used to treat beta thalassemia (β-Thal). As discussed elsewhere herein, the LNPs of the present disclosure can be used to treat hemoglobinopathies by delivering a gene editing system capable of correcting the HBB mutation or reactivating expression of fetal hemoglobin.

Provided herein are methods of treating a hemoglobinopathy in a patient in need thereof, the method comprising administering to the patient a therapeutically effective amount of a pharmaceutical composition of the present disclosure, comprising an LNP comprising a gene editing system of the present disclosure. In certain embodiments, the pharmaceutical composition is administered intravenously.

In certain embodiments, a therapeutically effective amount of a pharmaceutical composition of the present disclosure is a dosage regimen capable of affecting an edit to the hemoglobin or hemoglobin-associated gene or regulatory region in at least a portion of long-term hematopoietic stem cells in a bone marrow sample collected from the subject at a time point after administration. In certain embodiments, the portion of long-term hematopoietic stem cells in the sample possessing the edit is at least about 10%, about 15%, about 20%, about 25%, about 30%, about 40%, about 50%, about 60%, about 70%, about 80% or about 90%. In certain embodiments, the time point when the bone marrow sample is collected from the subject is about 7 days, about 14 days, about 21 days, about 28 days, about 50 days, about 100 days, about 1 year, about 2 years, about 5 years, or about 10 years. In certain embodiments, a therapeutically effective amount of a pharmaceutical composition of the present disclosure is a dosage regimen capable of affecting an edit to the hemoglobin or hemoglobin-associated gene or regulatory region in at least 25% of long-term hematopoietic stem cells in a bone marrow sample collected from the subject 28 days after administration.

Also provided herein are uses of pharmaceutical compositions of the present disclosure for the manufacture of a medicament for treating hemoglobinopathies. Further provided herein are pharmaceutical compositions for use in treating hemoglobinopathies. Additionally provided herein are uses of pharmaceutical compositions of the present disclosure for the manufacture of a medicament for treating hemoglobinopathies, wherein the medicament is prepared to be administered in a dosage regime effective to treat the hemoglobinopathy.

### Correction of HBB Mutation

In certain embodiments, the gene editing system treats the hemoglobinopathy by repairing the mutant β-globin gene. In general, strategies of repairing the β-globin gene in order to address a β-hemoglobinopathy are illustrated in the central panel of FIG. 1.

### Homology Directed Repair (HDR)

In certain embodiments, the gene editing system corrects the β-globin mutation through homology directed repair.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system that corrects a β-globin mutation through homology directed repair.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV), (V), (VI), (VII), (VIII), (IX), or (X); and
b) at least one gene editing system that corrects a β-globin mutation through homology directed repair.

In certain embodiments, the gene editing system comprises a CRISPR-Cas9 capable of removing the mutant HBB exon. In certain embodiments, the gene editing system comprises a nickase capable of removing the mutant HBB exon. In certain embodiments, the gene editing system comprises a TALEN capable of removing the mutant HBB exon. In certain embodiments, the gene editing system comprises a ZFN capable of removing the mutant HBB exon. In certain embodiments, the gene editing system comprises a donor template comprising a corrected or wild type HBB exon. In certain embodiments, the at least one gene editing system that corrects a β-globin mutation through homology directed repair is one described in a reference in Table X1.

### Non-Homologous End-Joining (NHEJ)

In certain embodiments, the gene editing system corrects the β-globin mutation through non-homologous end-joining (NHEJ), by targeting aberrant splicing sites in introns I and II, resulting in restoration of functional β-globin transcripts.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system that corrects a β-globin mutation through non-homologous end-joining (NHEJ).

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV), (V), (VI), (VII), (VIII), (IX), or (X); and
b) at least one gene editing system that corrects a β-globin mutation through non-homologous end-joining (NHEJ).

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising about 20 mol % to about 60 mol % phospholipid; and
b) at least one gene editing system that corrects a β-globin mutation through non-homologous end-joining (NHEJ).

In certain embodiments, the gene editing system comprises a CRISPR-Cas9 capable of targeting aberrant splicing sites in an intron of a mutated HBB. In certain embodiments, the gene editing system comprises a TALEN capable of targeting aberrant splicing sites in an intron of a mutated HBB. In certain embodiments, the at least one gene editing system that corrects a β-globin mutation through non-homologous end-joining (NHEJ) is one described in a reference in Table X1.

### Single Base Pair Correction

In certain embodiments, the gene editing system corrects the β-globin mutation through a single base pair gene correction. In certain embodiments, the gene editing system is a prime editing or base editing system capable of executing a single base pair edit. In certain embodiments, the gene editing system executes a single base pair correction in a SCD mutation, converting the aberrant thymine to the normal adenine, thereby converting the aberrant valine amino acid in the mutant hemoglobin to the wild-type glutamic acid. In certain embodiments, the gene editing system executes a single base pair correction, converting the aberrant thymine to cytosine, thereby converting the aberrant valine amino acid in the mutant hemoglobin to alanine, which results in a functional, nonpathogenic hemoglobin variant (HbG Makassar).

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system comprising a base editor capable of executing a single base pair edit in a mutant HBB gene.

In one aspect, the present disclosure provides a method of treating a β-hemoglobinopathy, the method comprising administering to a patient in need thereof a therapeutically effective amount of a pharmaceutical composition comprising:

a) at least one lipid nanoparticle (LNP) comprising at least one ionizable lipid selected from those listed in Tables (I), (II), (III), (IV), (V), (VI), (VII), (VIII), (IX), or (X); and

b) at least one gene editing system comprising a base editor capable of executing a single base pair edit in a mutant HBB gene.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising about 20 mol % to about 60 mol % phospholipid; and
b) at least one gene editing system comprising a base editor capable of executing a single base pair edit in a mutant HBB gene.

In certain embodiments, the gene editing system comprises an adenine base editor. In certain embodiments, the gene editing system comprises a base editor that converts aberrant thymine to the normal adenine, thereby correcting a mutant HBB to wild-type HBB. In certain embodiments, the gene editing system comprises a base editor that converts an aberrant thymine to cytosine resulting in HbG Makassar. In certain embodiments, the gene editing system comprises a base editor described in a reference in Table X1.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system comprising a prime editor capable of executing a single base pair edit in a mutant HBB gene.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV), (V), (VI), (VII), (VIII), (IX), or (X); and
b) at least one gene editing system comprising a prime editor capable of executing a single base pair edit in a mutant HBB gene.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising about 20 mol % to about 60 mol % phospholipid; and
b) at least one gene editing system comprising a prime editor capable of executing a single base pair edit in a mutant HBB gene.

In certain embodiments, the gene editing system comprises a prime editor that converts aberrant thymine to the normal adenine, thereby correcting a mutant HBB to wild-type HBB. In certain embodiments, the gene editing system comprises a prime editor that converts an aberrant thymine to cytosine resulting in HbG Makassar. In certain embodiments, the gene editing system comprises a prime editor described in a reference in Table X1.

### Reactivation of γ-Globin Production

In certain embodiments, the gene editing system reactivates γ-globin production. One method of treating SCD and beta-Thalassemia is to reactivate production of functional fetal hemoglobin (γ-globin) to compensate for the pathogenic mutant β-globin. This mutation occurs naturally and is known as hereditary persistence of fetal hemoglobin (HPFH). β-globin mutations can be treated by artificially inducing a second mutation that mimics HPFH in a patient. In general, strategies of reactivating production of functional fetal hemoglobin (γ-globin) in order to address a β-hemoglobinopathy are illustrated in the right panel of FIG. 1.

### Disruption of HbF Inhibitory Sequences

In certain embodiments, the gene editing system reactivates γ-globin production by deleting or disrupting inhibitory sequences, or portions thereof. In certain embodiments, the gene editing system eliminates an HbF inhibitory sequence, or a portion thereof, thereby reactivating γ-globin production.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system comprising at least one nuclease and one guide RNA, capable of deleting at least a portion of the HbF inhibitory sequence.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV), (V), (VI), (VII), (VIII), (IX), or (X); and
b) at least one gene editing system comprising at least one nuclease and one guide RNA, capable of deleting at least a portion of the HbF inhibitory sequence.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising about 20 mol % to about 60 mol % phospholipid; and
b) at least one gene editing system comprising at least one nuclease and one guide RNA, capable of deleting at least a portion of the HbF inhibitory sequence.

In certain embodiments, the gene editing system comprises a CRISPR/Cas9. In certain embodiments, the gene editing system comprises a CRISPR/Cas12a. In certain embodiments, the gene editing system comprises multiple guide RNAs targeting multiple points on the HbF inhibitor sequence, thereby enabling a large deletion. In certain embodiments, the gene editing system juxtaposes the γ-globin promoters to remote enhancer regions. In certain embodiments, the gene editing system comprises a gene editing system for deleting at least a portion of the HbF inhibitor sequence described in a reference in Table X1.

Disruption of HBG1 and/or HBG2 Promoters

In certain embodiments, the gene editing system targets the HBG1 and/or HBG2 promoters, resulting in disruption of binding sites for γ-globin transcriptional repressors, leading to HbF expression.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system comprising at least one nuclease and one guide RNA, capable of deleting or disrupting at least a portion of the HBG1 and/or HBG2 promoter sequences.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV), (V), (VI), (VII), (VIII), (IX), or (X); and
b) at least one gene editing system comprising at least one nuclease and one guide RNA, capable of deleting or disrupting at least a portion of the HBG1 and/or HBG2 promoter sequences.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising about 20 mol % to about 60 mol % phospholipid; and
b) at least one gene editing system comprising at least one nuclease and one guide RNA, capable of deleting or disrupting at least a portion of the HBG1 and/or HBG2 promoter sequences.

In certain embodiments, the gene editing system comprises a CRISPR/Cas9. In certain embodiments, the gene editing system comprises a CRISPR/Cas12a. In certain embodiments, the gene editing system comprises one or more base editors capable of disrupting the HBG1/2 promoters. In certain embodiments, the gene editing system comprises multiple guide RNAs targeting multiple points on the HBG1 and/or HBG2 promoters. In certain embodiments, the gene editing system comprises a gene editing system for deleting or disrupting at least a portion of the HBG1 and/or IIBG2 promoter sequences described in a reference in Table X1. In certain embodiments, the gene editing system is one disclosed or described in one of U.S. Pat. No. 11,466,271; US Application Publications US20200140896A1; US20220047637A1; and US20200399626A1; and PCT Publications WO2017/077394; WO2016/182917; WO2015/073683; WO2022/155458A1; and WO2021/163587A1.

### Disruption of GATA1 Binding Site

In certain embodiments, the gene editing system disrupts the GATA1 motif in the erythroid-specific intronic enhancer of BCL11A.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system comprising at least one nuclease and one guide RNA, capable of deleting or disrupting at least a portion of the GATA1 binding site in the BCL11A enhancer.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV), (V), (VI), (VII), (VIII), (IX), or (X); and
b) at least one gene editing system comprising at least one nuclease and one guide RNA, capable of deleting or disrupting at least a portion of the GATA1 binding site in the BCL11A enhancer.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising about 20 mol % to about 60 mol % phospholipid; and
b) at least one gene editing system comprising at least one nuclease and one guide RNA, capable of deleting or disrupting at least a portion of the GATA1 binding site in the BCL11A enhancer.

In certain embodiments, the gene editing system comprises a CRISPR/Cas9. In certain embodiments, the gene editing system comprises a CRISPR/Cas12a. In certain embodiments, the gene editing system comprises one or more base editors. In certain embodiments, the gene editing system comprises a gene editing system for deleting or disrupting at least a portion of the GATA1 binding site described in a reference in Table X1.

### Disruption of BCL11A Binding Site

In certain embodiments, the gene editing system comprises base editors that reproduce HPFH mutations by disrupting repressor binding sites (such as BCL11A). In certain embodiments, the gene editing system disrupts BCL11A binding sites.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system comprising at least one nuclease and one guide RNA, capable of deleting or disrupting at least a portion of the BCL11A binding site.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV), (V), (VI), (VII), (VIII), (IX), or (X); and
b) at least one gene editing system comprising at least one nuclease and one guide RNA, capable of deleting or disrupting at least a portion of the BCL11A binding site.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising about 20 mol % to about 60 mol % phospholipid; and
b) at least one gene editing system comprising at least one nuclease and one guide RNA, capable of deleting or disrupting at least a portion of the BCL11A binding site.

In certain embodiments, the gene editing system comprises a CRISPR/Cas9. In certain embodiments, the gene editing system comprises a CRISPR/Cas12a. In certain embodiments, the gene editing system comprises one or more base editors. In certain embodiments, the gene editing system comprises a gene editing system for deleting or disrupting at least a portion of the BCL11A binding site described in a reference in Table X1.

### Creation of Transcriptional Activator Binding Site

In certain embodiments, the gene editing system creates transcriptional activator binding sites. In certain embodiments, the gene editing system comprises base editors that reproduce HPFH mutations by creating binding sites for transcriptional activators (such as KLF1).

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV) or (V); and
b) at least one gene editing system comprising at least one nuclease and one guide RNA, capable of creating or activating a binding site for a transcriptional activator that promotes fetal hemoglobin production.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising at least one ionizable lipid selected from those listed in Tables (I), (II), (III), (IV), (V), (VI), (VII), (VIII), (IX), or (X); and
b) at least one gene editing system comprising at least one nuclease and one guide RNA, capable of creating or activating a binding site for a transcriptional activator that promotes fetal hemoglobin production.

In one aspect, the present disclosure provides a method of treating a β-hemoglobinopathy, the method comprising administering to a patient in need thereof a therapeutically effective amount of a pharmaceutical composition comprising:
a) at least one lipid nanoparticle (LNP) comprising about 20 mol % to about 60 mol % phospholipid; and
b) at least one gene editing system comprising at least one nuclease and one guide RNA, capable of creating or activating a binding site for a transcriptional activator that promotes fetal hemoglobin production.

In certain embodiments, the gene editing system comprises a CRISPR/Cas9. In certain embodiments, the gene editing system comprises a CRISPR/Cas12a. In certain embodiments, the gene editing system comprises one or more base editors. In certain embodiments, the gene editing system comprises a gene editing system capable of creating or activating a binding site for a transcriptional activator that promotes fetal hemoglobin production described in a reference in Table X1. In certain embodiments, the gene editing system comprises one or more components disclosed in Zhou, et al. KLF1 regulates BCL11A expression and γ- to β-globin gene switching. Nat. Genet. 2010, 42, 742-744.

D. Co-Therapy with Another Therapeutic Agent

In other embodiments, LNP-based therapeutics and pharmaceutical compositions thereof described herein may be co-administered in conjunction with another agent for treating a disease (e.g., a hemoglobinopathy). In certain embodiments, the LNP therapeutics disclosed herein are co-administered in conjunction with a standard of treatment medication normally administered to patients suffering from sickle cell disease. In certain embodiments, the co-administered medication is one or more selected from Hydroxyurea (DROXIA, HYDREA, SIKLOS), L-glutamine oral powder (ENDARI), Crizanlizumab (ADAKVEO), Voxelotor (OXBRYTA) and pain-relieving medications.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

As used herein, the following terms and phrases are intended to have the following meanings:

### A/an

The articles “a” and “an” are used herein to refer to one or to more than one (i.e., to at least one) of the grammatical object of the article. By way of example, “an element” means one element or more than one element.

### About

As used herein, the term “about” means acceptable variations within 20%, within 10% and within 5% of the stated value. In certain embodiments, “about” can mean a variation of +/−1%, 2%, 3%, 4%, 5%, 10% or 20%. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

### Adjuvant

As used herein, “adjuvant” means an agent that does not constitute a specific antigen, but modifies (Th1/Th2), boosts the strength and longevity of an immune response, and/or broadens the immune response to a concomitantly administered antigen.

### Administration

The term “administration” or “administering” as used herein includes all means of introducing the compounds or the pharmaceutical compositions to the subject in need thereof, including but not limited to, oral, intravenous, intramuscular, intraperitoneal, subcutaneous, transdermal, inhalation, buccal, ocular, sublingual, vaginal, rectal and the like. Administration of the compound or the composition is suitably parenteral. For example, the compounds or the composition can be preferentially administered intravenously, but can also be administered intraperitoneally or via inhalation like is currently used in the clinic for liposomal amikacin in the treatment of Mycobacterium avium (see Shirley et al., Amikacin Liposome Inhalation Suspension: A Review in Mycobacterium avium Complex Lung Disease. Drugs. 2019 April; 79(5):555-562).

### Aliphatic

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule, or two points of attachment to the rest of the molecule, as would be apparent to a person of ordinary skill in the art based on the context of the relevant molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

### Alkenyl

As used herein, “alkenyl” means a straight chain, cyclic or branched aliphatic hydrocarbon having the specified number of carbon atoms and one or more double bonds including but not limited to diene, triene and tetraene unsaturated aliphatic hydrocarbons. The terms “alkenyl” and “alkynyl”, refer to unsaturated aliphatic groups analogous in length and possible substitution to the alkyls described above, but that contain at least one double or triple bond respectively. In one embodiment, the alkenyl contains one double bond. In another embodiment, the alkenyl contains two double bonds. In another embodiment, the alkenyl contains three double bonds.

### Alkenylenyl

The term “alkenylenyl” as used herein refers to a divalent radical of an alkenyl group. In one embodiment, the alkenylenyl is a divalent form of a C₂₋₁₂ alkenyl, i.e., a C₂-C₁₂ alkenylenyl. In one embodiment, the alkenylenyl is a divalent form of a C₂₋₆ alkenyl, i.e., a C₂-C₁₀ alkenylenyl. In one embodiment, the alkenylenyl is a divalent form of a C₂₋₁₄ alkenyl, i.e., a C₂-C₈ alkenylenyl. In one embodiment, the alkenylenyl is a divalent form of an unsubstituted C₂₋₆ alkenyl, i.e., a C₂-C₆ alkenylenyl. In another embodiment, the alkylenyl is a divalent form of an unsubstituted C₂₋₄ alkenyl, i.e., a C₂-C₄ alkenylenyl. Nonlimiting exemplary alkenylenyl groups include CH═CH—, CH₂CH═CH—, CH₂CH₂CH═CHCH₂—, and CH₂CH═CHCH₂CH═CHCH₂CH₂—.

### Alkoxyl

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

### Alkyl

As used herein, “alkyl” means a straight chain, cyclic or branched saturated aliphatic hydrocarbon having the specified number of carbon atoms.

### Alkylenyl

The term “alkylenyl” as used herein refers to a divalent radical of a straight-chain or branched-chain alkyl group. In one embodiment, the alkylenyl is a divalent form of a C₁₋₁₂ alkyl, i.e., a C₁-C₁₂ alkylenyl. In one embodiment, the alkylenyl is a divalent form of a C₂₋₆ alkyl, i.e., a C₁-C₁₀ alkylenyl. In one embodiment, the alkylenyl is a divalent form of a C₂₋₁₄ alkyl, i.e., a C₁-C₈ alkylenyl. In one embodiment, the alkylenyl is a divalent form of an unsubstituted C₁₋₆ alkyl, i.e., a C₁-C₆ alkylenyl. In another embodiment, the alkylenyl is a divalent form of an unsubstituted C₁₋₄ alkyl, i.e., a C₁-C₄ alkylenyl. Nonlimiting exemplary alkylenyl groups include CH₂—, CH₂CH₂—, CH₂CH₂CH₂—, CH₂CH(CH₃)CH₂—, —CH₂ (CH₂)₂CH₂—, CH(CH₂)₃CH₂—, and CH₂(CH₂)₄CH₂—.

### Alkylthio

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

### Aralkyl

The term “aralkyl,” as used herein, refers to an alkyl group substituted with an aryl group (e.g., an aromatic or heteroaromatic group).

### Aryl

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

### Analogs

As used herein, “analogs” is meant to include polypeptide variants which differ by one or more amino acid alterations, for example, substitutions, additions or deletions of amino acid residues that still maintain one or more of the properties of the parent or starting polypeptide.

### Antibodies

As used herein, the term “antibody” is referred to in the broadest sense and specifically covers various embodiments including, but not limited to monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include VH and VL domains, scFvs, Fab, Fab′, F(ab′)2, Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

### Antigen

As defined herein, the term “antigen” or “antibody generator” (“Ag”) refers to a composition, for example, a substance or agent which causes an immune response in an organism, e.g., causes the immune response of the organism to produce antibodies against the substance or agent, in particular, which provokes an adaptive immune response in an organism. Antigens can be any immunogenic substance including, in particular, proteins, polypeptides, polysaccharides, nucleic acids, lipids and the like. Exemplary antigens are derived from infectious agents. Such agents can include parts or subunits of infectious agents, for example, coats, coat components, e.g., coat protein or polypeptides, surface components, e.g., surface proteins or polypeptides, capsule components, cell wall components, flagella, fimbrae, and/or toxins or toxoids) of infectious agents, for example, bacteria, viruses, and other microorganisms. Certain antigens, for example, lipids and/or nucleic acids are antigenic, preferably, when combined with proteins and/or polysaccharides.

### Approximately

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

### Associated

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

### Base Editor

As used herein the term “base editor” refers to a gene editing system comprising the following minimal components: a nucleic acid programmable nuclease (e.g., a CRISPR type II nuclease), one or more deaminases, and a guide RNA. The nucleic acid programmable nuclease may

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

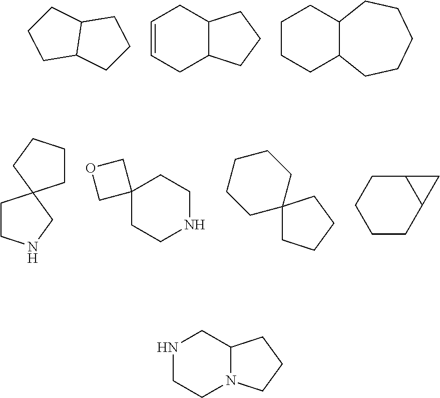

Exemplary bridged bicyclics include:

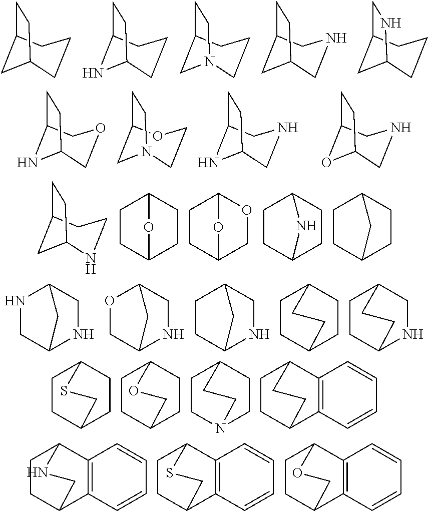

### Biologically Active

As used herein, the phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present disclosure may be considered biologically active if even a portion of the polynucleotides is biologically active or mimics an activity considered biologically relevant.

### Binding Domain

By “binding domain” it is meant a protein domain that is able to bind non-covalently to another molecule. A binding domain can bind to, for example, a DNA molecule (a DNA-binding domain), an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

### Bulge

As used herein, the term “bulge” refers to a small region of unpaired base(s) that interrupts a “stem” of base-paired nucleotides. The bulge may comprise one or two single-stranded or unbase-paired nucleotides joined at both ends by base-paired nucleotides of the stem. The bulge can be symmetrical (viz., the two unbase-paired single-stranded regions have the same number of nucleotides), or asymmetrical (viz., the unbase-paired single stranded region(s) have different or unequal numbers of nucleotides), or there is only one unbase-paired nucleotide on one strand. A bulge can be described as A/B (such as a “2/2 bulge,” or a “1/0 bulge”) wherein A represents the number of unpaired nucleotides on the upstream strand of the stem, and B represents the number of unpaired nucleotides on the downstream strand of the stem. An upstream strand of a bulge is more 5′ to a downstream strand of the bulge in the primary nucleotide sequence.

### CARs

As used herein, the term “chimeric antigen receptor” or “CAR” refers to an artificial chimeric protein comprising at least one antigen specific targeting region (ASTR), a transmembrane domain and an intracellular signaling domain, wherein the antigen specific targeting region comprises a full-length antibody or a fragment thereof. Any molecule that is capable of binding a target antigen with high affinity can be used in the ASTR of a CAR. The CAR may optionally have an extracellular spacer domain and/or a co-stimulatory domain. A CAR may also be used to generate a cytotoxic cell carrying the CAR.

### Carbocycle

The term “carbocycle,” as used herein, refers to an aromatic or non-aromatic ring in which each atom of the ring is carbon.

### Carbonyl

The term “carbonyl” is art-recognized and includes such moieties as can be represented by the general formula:

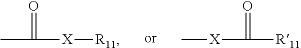

wherein X is a bond or represents an oxygen or a sulfur, and R₁₁ represents a hydrogen, an alkyl, a cycloalkyl, an alkenyl, a cycloalkenyl, or an alkynyl, R′₁₁ represents a hydrogen, an alkyl, a cycloalkyl, an alkenyl, a cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R¹¹ is hydrogen, the above formula represents an “aldehyde” group.

### Cargo or Payload

As used herein, the term “cargo” or “payload” can refer to one or more molecules or structures encompassed in a delivery vehicle for delivery to or into a cell or tissue. Non-limiting examples of cargo can include a nucleic acid (e.g., mRNA, such as a linear or a circular mRNA), a polypeptide, a peptide, a protein, a liposome, a label, a tag, a small chemical molecule, a large biological molecule, and any combinations thereof.

### Cationic Lipid

As used herein, “cationic lipid” refers to any of a number of lipid species which carry a net positive charge at a selective pH, such as physiological pH.

### Cas12a or Cas12a Polypeptide

As used herein, the “Cas12a polypeptide”, “Cas12a protein” or “Cas12a nuclease” refers to a RNA-binding site-directed CRISPR Cas TypeV polypeptide that recognizes and/or binds RNA and is targeted to a specific DNA sequence. An Cas12a system as described herein refers to a specific DNA sequence by the RNA molecule to which the Cas12a polypeptide or Cas12a protein is bound. The RNA molecule comprises a sequence that binds, hybridizes to, or is complementary to a target sequence within the targeted polynucleotide sequence, thus targeting the bound polypeptide to a specific location within the targeted polynucleotide sequence (the target sequence). “Cas12a” is a type of CRISPR Class II Type V nuclease. The specification may describe the polypeptides contemplated in the scope of this application as Cas12a polypeptides or alternatively as Cas TypeV polypeptides, or the like.

cDNA

As used herein, the term “cDNA” refers to a strand of DNA copied from an RNA template, e.g., by a reverse transcriptase.

### Circular RNA

As used herein, the terms “circular RNA” or “circRNA” or “ORNA” equivalently refer to a RNA that forms a circular structure through covalent or non-covalent bonds.

### Cleavage

As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends.

### Cognate

The term “cognate” refers to two biomolecules that normally interact or co-exist in nature.

### CRISPR-Type II Gene Editing System

As used herein the term “CRISPR-type II gene editing system” refers to a set of components capable of conducting nucleic acid programmable gene editing that minimally comprises a CRISPR type II nucleic acid programmable nuclease (e.g., Cas9 or homolog or variant thereof, e.g., a dead Cas9 or Cas9 nickase) and a guide RNA which complexes with said nuclease. The guide RNA is capable of complexing with a CRISPR type II nuclease and directing the complex to a target DNA site that is complementary to the spacer sequence of the guide. This term also embraces systems comprising additional accessory proteins, such as another nuclease, reverse transcriptase, deaminase, recombinase, transposase, integrase, DNA binding protein, transcription factor, a CRISPR Cas protein, or an anti-CRISPR inhibitor protein (Hwang and Maxwell, The CRISPR Journal; Volume 2, Number 1, 2019, which is incorporated by reference herein in its entirety). In one embodiment, a CRISPR-type II gene editing system can be a base editor comprising CRISPR type II nucleic acid programmable nuclease (e.g., Cas9 or homolog or variant thereof, e.g., a dead Cas9 or Cas9 nickase), a guide RNA, and one or more deaminases, wherein the one or more deaminases is optionally fused to the nuclease via a linker. In another embodiment, a CRISPR-type II gene editing system can be a prime editor comprising CRISPR type II nucleic acid programmable nuclease (e.g., Cas9 or homolog or variant thereof, e.g., a dead Cas9 or Cas9 nickase), a prime editing guide RNA (pegRNA), and a reverse transcriptase, wherein the reverse transcriptase is optionally fused to the nuclease via a linker. In various embodiments, the CRISPR-type II gene editing system components, such as the nuclease, guide RNAs, or accessory proteins, may comprise one or more NLSs to facilitate their transfer into the nucleus. The gene editing system may be in the form of DNA (encoding the protein components and the guide RNA), RNA (encoding the protein components and constituting a guide RNA), or a mixture of protein components (e.g., a nuclease) and RNA (e.g., a guide RNA).

### CRISPR-Type V Gene Editing System

As used herein the term “CRISPR-type V gene editing system” refers to a set of components capable of conducting nucleic acid programmable gene editing that minimally comprises a CRISPR type V nucleic acid programmable nuclease (e.g., Cas12a or homolog or variant, e.g., a dead Cas12a or Cas12a nickase) and a guide RNA which complexes with said nuclease. The guide RNA is capable of complexing with a CRISPR type V nuclease and directing the complex to a target DNA site that is complementary to the spacer sequence of the guide. In one embodiment, a CRISPR-type V gene editing system can be a base editor comprising CRISPR type V nucleic acid programmable nuclease (e.g., Cas12a or homolog or variant thereof, e.g., a dead Cas12a or Cas12a nickase), a guide RNA, and one or more deaminases, wherein the one or more deaminases is optionally fused to the nuclease via a linker. In another embodiment, a CRISPR-type V gene editing system can be a prime editor comprising CRISPR type V nucleic acid programmable nuclease (e.g., Cas12a or homolog or variant thereof, e.g., a dead Cas12a or Cas12a nickase), a prime editing guide RNA (pegRNA), and a reverse transcriptase, wherein the reverse transcriptase is optionally fused to the nuclease via a linker. In various embodiments, the CRISPR-type V gene editing system components, such as the nuclease, guide RNAs, or accessory proteins, may comprise one or more NLSs to facilitate their transfer into the nucleus. The gene editing system may be in the form of DNA (encoding the protein components and the guide RNA), RNA (encoding the protein components and constituting a guide RNA), or a mixture of protein components (e.g., a nuclease) and RNA (e.g., a guide RNA).

### Co-Administration

As used herein the term “co-administration” or “co-administering” refers to administration of the LNP adjuvant and an agonist or antigen concurrently, i.e., simultaneously in time, or sequentially, i.e., administration of an LNP adjuvant, followed by administration of the agonist or antigen. That is, after administration of the LNP adjuvant, the agonist or antigen can be administered substantially immediately after the LNP adjuvant or the agonist or antigen can be administered after an effective time period after the LNP adjuvant; the effective time period is the amount of time given for realization of maximum benefit from the administration of the LNP adjuvant. An effective time period can be determined experimentally and can be generally within 1, 2, 3, 5, 10, 15, 20, 25, 30, 45 or 60 minutes.

### Complementary

As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

### Compound or Structure

The term “compound” or “structure,” as used herein, is meant to include all stereoisomers, geometric isomers, tautomers, and isotopes of the structures depicted. The compounds or structures described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds or structures of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds or structures of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g., have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. In some embodiments, alkyl groups are lower alkyls. In some embodiments, a substituent designated herein as alkyl is a lower alkyl.

### Comprising/Comprises

As used herein the term “comprising” or “comprises” is used in reference to compositions, methods, and respective component(s) thereof, that are present in a given embodiment, yet open to the inclusion of unspecified elements.

### Conservative Amino Acid Substitution

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

### Consisting Essentially of

As used herein the term “consisting essentially of” refers to those elements required for a given embodiment. The term permits the presence of additional elements that do not materially affect the basic and novel or functional characteristic(s) of that embodiment of the disclosure.

### Consisting of

The term “consisting of” refers to compositions, methods, and respective components thereof as described herein, which are exclusive of any element not recited in that description of the embodiment.

### Cycloalkylenyl

The term “cycloalkylenyl” as used herein refers to a divalent radical of a cycloalkyl group. In one embodiment, the cycloalkylenyl is a divalent form of a C₃₋₈ cycloalkyl, i.e., a C₃-C₈ cycloalkylenyl. Nonlimiting exemplary cycloalkylenyl groups include:

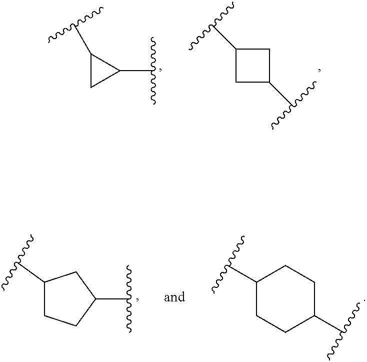

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

### Delivery

As used herein, “delivery” refers to the act or manner of delivering a compound, substance, entity, moiety, cargo or payload.

### Derivative

The present disclosure provides several types of compositions that are polynucleotide or polypeptide based, including variants and derivatives. These include, for example, substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

### DNA/RNA

As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

### DNA-Guided Nuclease or Nucleic Acid Programmable Nuclease

As used herein, an “DNA-guided nuclease” is a type of “programmable nuclease,” and a specific type of “nucleic acid-guided nuclease.” Equivalent terms for purposes of this disclosure include “nucleic acid programmable nuclease.” An example of a DNA-guided nuclease is reported in Varshney et al., DNA-guided genome editing using structure-guided endonucleases, Genome Biology, 2016, 17(1), 187, which may be used in the context of the present disclosure and is incorporated herein by reference. As used herein, the term “DNA-guided nuclease” or “DNA-guided endonuclease” refers to a nuclease that associates covalently or non-covalently with a guide RNA thereby forming a complex between the guide RNA and the DNA-guided nuclease. The guide RNA comprises a spacer sequence which comprises a nucleotide sequence having complementarity with a strand of a target DNA sequence. Thus, the DNA-guided nuclease is indirectly guided or programmed to localize to a specific site in a DNA molecule through its association with the guide RNA, which directly binds or anneals to a strand of the target DNA through its complementarity region via Watson-Crick base-pairing. A “nucleic acid programmable

### DNA-Guided DNA Binding Protein or Nucleic Acid Programmable DNA Binding Protein

As used herein, an “DNA-guided DNA binding protein” or “nucleic acid programmable DNA binding protein” has a similar meaning as a “DNA-guided nuclease” except that a “DNA binding protein” may include a nuclease but is not required to have a nuclease activity. By contrast to a nuclease, a nuclease domain (e.g., FokI) could be fused to a DNA binding protein top give rise to a DNA-guided nuclease as a fusion protein.

### DNA Regulatory Sequences

As used herein, the terms “DNA regulatory sequences,” “control elements,” and “regulatory elements,” can be used interchangeably herein to refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence and/or regulate translation of a mRNA into an encoded polypeptide.

### Domain

As used herein when referring to polypeptides the term “domain” refers to a motif of a polypeptide having one or more identifiable structural or functional characteristics or properties (e.g., binding capacity, serving as a site for protein-protein interactions).

### Donor Template DNA or Template DNA

By a “donor template DNA” or “donor DNA” or “template DNA” it is meant a single-stranded or double-stranded DNA to be incorporated at a site cleaved by a programmable nuclease (e.g., a CRISPR/Cas effector protein; a TALEN; a ZEN; a meganuclease) (e.g., after dsDNA cleavage, after nicking a target DNA, after dual nicking a target DNA, and the like). The donor DNA can contain sufficient homology to a genomic sequence at the target site, e.g. 70%, 80%, 85%, 90%, 95%, or 100% homology with the nucleotide sequences flanking the target site, e.g., within about 200 bases or less of the target site, e.g., within about 190 bases or less of the target site, e.g., within about 180 bases or less of the target site, e.g., within about 170 bases or less of the target site, e.g., within about 160 bases or less of the target site, e.g., within about 150 bases or less of the target site, e.g., within about 140 bases or less of the target site, e.g., within about 130 bases or less of the target site, e.g., within about 120 bases or less of the target site, e.g., within about 110 bases or less of the target site, e.g., within about 100 bases or less of the target site, e.g., within about 90 bases or less of the target site, e.g., within about 80 bases or less of the target site, e.g., within about 70 bases or less of the target site, e.g., within about 60 bases or less of the target site, e.g., 50 bases or less of the target site, e.g., within about 30 bases, within about 15 bases, within about 10 bases, within about 5 bases, or immediately flanking the target site, to support integration into the cut site. In certain embodiments, integration of the donor template DNA occurs by way of homology-directed repair between the donor and the genomic sequence to which it bears homology.

### Encapsulate

The terms “encapsulation” and “entrapped,” as used herein, refer to the incorporation or association of the mRNA, DNA, siRNA or other nucleic acid pharmaceutical agent in or with a lipidic nanoparticle. As used herein, the term “encapsulated” refers to complete encapsulation or partial encapsulation. A siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. A siRNA may comprise a sequence that is complementary to an mRNA sequence that encodes a gene or protein of interest.

### Encapsulation Efficiency

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of a polynucleotide are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Throughout the disclosure, chemical substituents described in Markush structures are represented by variables. Where a variable is given multiple definitions as applied to different Markush formulas in different sections of the disclosure, it is to be understood that each definition should only apply to the applicable formula in the appropriate section of the disclosure.

### Encode

As used herein the term “encode” refers broadly to any process whereby the information in a polymeric macromolecule is used to direct the production of a second molecule that is different from the first. The second molecule may have a chemical structure that is different from the chemical nature of the first molecule.

### Exosome

As used herein, the term “exosomes” refer to small membrane bound vesicles with an endocytic origin. Without wishing to be bound by theory, exosomes are generally released into an extracellular environment from host/progenitor cells post fusion of multivesicular bodies the cellular plasma membrane. As such, exosomes can include components of the progenitor membrane in addition to designed components (e.g. engineered TnpB editing system). Exosome membranes are generally lamellar, composed of a bilayer of lipids, with an aqueous inter-nanoparticle space.

### Features

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini or any combination thereof.

### Fusion

The term “fusion” as used herein as applied to a nucleic acid or polypeptide refers to two components that are defined by structures derived from different sources. For example, where “fusion” is used in the context of a fusion polypeptide (e.g., a fusion Cas9-RT protein), the fusion polypeptide includes amino acid sequences that are derived from different polypeptides. A fusion polypeptide may comprise either modified or naturally-occurring polypeptide sequences (e.g., a first amino acid sequence from a modified or unmodified Cas9-RT protein; and a second amino acid sequence from a modified or unmodified protein other than a Cas9-RT protein, etc.). Similarly, “fusion” in the context of a polynucleotide encoding a fusion polypeptide includes nucleotide sequences derived from different coding regions (e.g., a first nucleotide sequence encoding a modified or unmodified Cas9-RT protein; and a second nucleotide sequence encoding a polypeptide other than a Cas9-RT protein).

### Fusion Polypeptide

The term “fusion polypeptide” refers to a polypeptide which is made by the combination (i.e., “fusion”) of two otherwise separated segments of amino acid sequence, usually through human intervention.

### Formulation

As used herein, a “formulation” includes at least one compound, substance, entity, moiety, cargo or payload and a delivery agent.

### Fragment

A “fragment,” as used herein, refers to a portion. For example, fragments of proteins may comprise polypeptides obtained by digesting full-length protein isolated from cultured cells.

### Halogen

As used herein, “halogen” means Br, Cl, F and I.

### Hemaglobinopathy

As used herein, the term hemoglobinopathy is a medical term for a group of inherited blood disorders that affect hemoglobin, the protein in red blood cells. Hemoglobinopathies are caused by genetic mutations that cause abnormal hemoglobin production or structure. Hemoglobinopathies are usually inherited as autosomal co-dominant traits. This group of disorders includes hemoglobin C disease, hemoglobin S—C disease, sickle cell disease (SCD), and beta-thalassemias, congenital sideroblastic anemia, and congenital dyserythropoietic anemia. The molecular mechanisms of hemaglobinopathies are well known and described in the medical and scientific literature, such as, Harteveld et al., “The hemoglobinopathies, molecular disease mechanisms and diagnostics,” Int. J. Lab. Hematol., September 2022; 44(Suppl. 1): 28-36, the entire contents of which are incorporated herein by reference.

### Hematopoietic Stem Cell (HSC)

As used in this application, the term “hematopoietic stem cell” or HSC is a multipotent stem cell type that is a progenitor of blood cells, including red blood cells (RBCs). HSCs differentiate through the process of erythropoiesis—a tightly-regulated and complex process originating in the bone marrow from an HSC and terminating in a mature, enucleated erythrocyte—which involves the formation of numerous distinct cell types. In human adults, erythropoiesis largely takes place in the bone marrow. Without wishing to be bound by theory, HSC first differentiate to form BFU-E cells, which differentiate to form CFU-E cells, which then differentiate to form proerythroblast cells, which differentiate to form basophilic erythroblast cells, which differentiate to form polychromatic erythroblast cells, which differentiate to form orthochromatic erythroblast cells, which differentiate to form reticulocytes, which are released into the blood stream and further mature by losing its internal organelles and remodeling its plasma membrane to form a red blood cell. Erythropoiesis is one type of hematopoiesis, which refers more broadly to the body's process of the differentiation of HSCs in the bone marrow to form common myeloid progenitor cells (which then differentiation into red blood cells (erythropoiesis) and thrombocytes/platelets (thrombopoiesis) and common lymphoid progenitors (which then differentiate into all types of white blood cells, including natural killer cells, T lymphocytes, and B lymphocytes). It should be noted that the LNPs described herein, in some embodiments, may be specifically delivered to any of the cell types formed during erythropoiesis, including but not limited to, HSCs, BFU-E cells, CFU-E cells, proerythroblast cells, basophilic crythroblast cells, polychromatic erythroblast cells, crythroblast cells, and reticulocytes. In addition, it is noted that the process and bodily location of erythropoiesis changes from fetus (yolk to liver then to bone marrow) to adult (bone marrow in a changing set of bones with time). The LNPs described herein may also be specifically delivered to any of the cell types that are formed during erythropoiesis during any stage of life, including early fetal erythropoiesis to later fetal erythropoiesis to adult erythropoiesis. These cells types including, but are not limited to HSCs, BFU-E cells, CFU-E cells, proerythroblast cells, basophilic erythroblast cells, polychromatic erythroblast cells, erythroblast cells, and reticulocytes, as well as erythroid progenitor cells, myeloid progenitor cells, hematopoietic stem cells (HSCs), erythrocytes, long-term HSCs (LT-HSC), short-term HSCs (ST-HSC), multipotent progenitors (MPP). In certain embodiments, the cell type of interest is common myeloid progenitors (CMP), and megakaryocyte erythroid progenitors (MEP). \

### Heteroalkyl

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

### Heteroatom

The term “heteroatom” as used herein means an atom of any element other than carbon or hydrogen. Examples of heteroatoms are boron, nitrogen, oxygen, phosphorus, sulfur and selenium. Other useful heteroatoms include silicon and arsenic.

### Heterocyclyl

As used herein, “heterocyclyl” or “heterocycle” means a 4- to 10-membered aromatic or nonaromatic heterocycle containing from 1 to 4 heteroatoms selected from the group consisting of O, N and S, and includes bicyclic groups. “Heterocyclyl” therefore includes, the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazctidinyl, methylenedioxybenzoyl, tetrahydrofuranyl, and tetrahydrothienyl, and N-oxides thereof all of which are optionally substituted with one to three substituents selected from R″.

### Heterocycle

“Heterocycle” or “heterocyclic,” as used herein, refers to a cyclic radical attached via a ring carbon or nitrogen of a monocyclic or bicyclic ring containing 3-10 ring atoms, for example, from 5-6 ring atoms, consisting of carbon and one to four heteroatoms each selected from the group consisting of non-peroxide oxygen, sulfur, and N (Y) wherein Y is absent or is H, O, (C1-C10) alkyl, phenyl or benzyl, and optionally containing 1-3 double bonds and optionally substituted with one or more substituents. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, and —CN.

### Homology

As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids. Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” arc genes (or proteins) related by duplication within a genome. Orthologs retain the same function in the course of evolution, whereas paralogs evolve new functions, even if these are related to the original one.

### Homology-Directed Repair

As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the targeted polynucleotide sequence.

### Identity

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

### Immunogenic

As used herein, the term “immunogenic” refers to a potential to induce an immune response to a substance. An immune response may be induced when an immune system of an organism or a certain type of immune cell is exposed to an immunogenic substance. The term “non-immunogenic” refers to a lack of or absence of an immune response above a detectable threshold to a substance. No immune response is detected when an immune system of an organism or a certain type of immune cell is exposed to a non-immunogenic substance. In some embodiments, a non-immunogenic circular polyribonucleotide as provided herein, does not induce an immune response above a pre-determined threshold when measured by an immunogenicity assay. In some embodiments, no innate immune response is detected when an immune system of an organism or a certain type of immune cell is exposed to a non-immunogenic circular polyribonucleotide as provided herein. In some embodiments, no adaptive immune response is detected when an immune system of an organism or a certain type of immune cell is exposed to a non-immunogenic circular polyribonucleotide as provided herein.

### Ionizable Lipid

As used herein “ionizable lipid” refers to any of a number of lipid species that carry a net positive charge at a selected pH.

### IRES

As used herein, the term “internal ribosome entry site” or “IRES” refers to an RNA sequence or structural element ranging in size form 10 nucleotides to 1,000 nucleotides or more which is capable of initiating translation of a polypeptide in the absence of a normal RNA cap structure. In other words, IRES are sequences that can recruit ribosomes and allow cap-independent translation, which can link two coding sequences in one bicistronic vector and allow the translation of both proteins. See Kozak M, “A second look at cellular mRNA sequences said to function as internal ribosome entry sites,” Nucleic Acids Research, 2005, Vol. 33: pp. 6593-6602 (incorporated herein by reference).

### Linker

As used herein, the term “linker” refers to a molecule linking or joining two other molecules or moieties. The linker can be an amino acid sequence in the case of a linker joining two fusion proteins. For example, a TnpB protein can be fused to an accessory protein (e.g., a deaminase, nuclease, ligase, reverse transcriptase, recombinase, etc.) by an amino acid linker sequence. The linker can also be a nucleotide sequence in the case of joining two nucleotide sequences together. For example, in the instant case, a reRNA at its 5′ and/or 3′ ends may be linked by a nucleotide sequence linker to one or more other functional nucleic acid molecules, such as guide RNAs or HDR donor molecules. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

### Lipid Conjugate

The term “lipid conjugate” refers to a conjugated lipid that inhibits aggregation of lipid particles. Such lipid conjugates include, but are not limited to, polysarcosine (see e.g. WO2021191265A1 which is herein incorporated by reference in its entirety for all purposes), polyamide oligomers (e.g., ATTA-lipid conjugates), PEG-lipid conjugates, such as PEG coupled to di alkyl oxy propyls, PEG coupled to diacylglycerols, PEG coupled to cholesterol, PEG coupled to phosphatidylethanolamines, PEG conjugated to ceramides (see, e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

### Lipid Nanoparticle (LNP)

The term “lipid nanoparticle”, or “LNP”, refers to particles having a diameter of from about 5 to 500 nm. In some embodiments, lipid nanoparticle refers to any lipid composition that can be used to deliver a prophylactic product, preferably vaccine antigens, including, but not limited to, liposomes or vesicles, wherein an aqueous volume is encapsulated by amphipathic lipid bilayers (e.g., single; unilamellar or multiple; multilamellar), or, in other embodiments, wherein the lipids coat an interior comprising a prophylactic product, or lipid aggregates or micelles, wherein the lipid encapsulated therapeutic product is contained within a relatively disordered lipid mixture. Except where noted, the lipid nanoparticle does not need to have antigen incorporated therein and may be used to deliver a prophylactic product when in the same formulation.

In some embodiments, the active agent (e.g., RNA payload encoding a polypeptide, such as an antigen or therapeutic protein) is encapsulated into the LNP. In some embodiments, the active agent can be an anionic compound, for example, but not limited to DNA, RNA, natural and synthetic oligonucleotides (including antisense oligonucleotides, interfering RNA and small interfering RNA), nucleoprotein, peptide, nucleic acid, ribozyme, DNA-containing nucleoprotein, such as an intact or partially deproteinated viral particles (virions), oligomeric and polymeric anionic compounds other than DNA (for example, acid polysaccharides and glycoproteins)). In some embodiments, the active agent can be intermixed with an adjuvant.

In a LNP vaccine product described herein, the active agent is generally contained in the interior of the LNP. In some embodiments, the active agent comprises a nucleic acid (e.g., a circular or linear mRNA). Typically, water soluble nucleic acids are condensed with cationic lipids or polycationic polymers in the interior of the particle and the surface of the particle is enriched in neutral lipids or PEG-lipid derivatives. Additional ionizable cationic lipid may also be at the surface and respond to acidification in the environment by becoming positively charged, facilitating endosomal escape. Lipid components of the herein disclosed LNPs are described herein.

Release of nucleic acids from LNP formulations, among other characteristics such as liposomal clearance and circulation half-life, can be modified by the presence of polyethylene glycol and/or sterols (e.g., cholesterol) or other potential additives in the LNP, as well as the overall chemical structure, including pKa of any ionizable cationic lipid included as part of the formulation.

### Liposome

As used herein “liposome” generally refers to a vesicle composed of lipids (e.g., amphiphilic lipids) arranged in one or more spherical bilayers or bilayers.

### Modified

As used herein “modified” refers to a changed state or structure of a molecule. Molecules may be modified in many ways including chemically, structurally, and functionally.

### Modulating

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

### Naturally-Occurring

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism that can be isolated from a source in nature is naturally occurring.

### Non-Homologous End Joining

As used herein, “non-homologous end joining (NHEJ)” refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

### Nuclear Localization Sequence (NLS)

As used herein, the term “nuclear localization sequence” or “NLS” refers to an amino acid sequence that promotes import of a protein (e.g., a RNA-guided nuclease) into the cell nucleus, for example, by nuclear transport. Nuclear localization sequences are known in the art. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences.

### Nuclease

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

### Nucleic Acid

As used herein, the term “nucleic acid” or “nucleic acid molecule” or “nucleic acid sequence” or “polynucleotide” generally refer to deoxyribonucleic or ribonucleic oligonucleotides in either single- or double-stranded form. The term may (or may not) encompass oligonucleotides containing known analogues of natural nucleotides. The term also may (or may not) encompass nucleic acid-like structures with synthetic backbones, see, e.g., Eckstein, 1991; Baserga et ah, 1992; Milligan, 1993; WO 97/03211; WO 96/39154; Mata, 1997; Strauss-Soukup, 1997; and Samstag, 1996. The term encompasses both ribonucleic acid (RNA) and DNA, including cDNA, genomic DNA, synthetic, synthesized (e.g., chemically synthesized) DNA, and/or DNA (or RNA) containing nucleic acid analogs. The nucleotides Adenine (A), Thymine (T), Guanine (G) and Cytosine (C) also may (or may not) encompass nucleotide modifications, e.g., methylated and/or hydroxylated nucleotides, e.g., Cytosine (C) encompasses 5-methylcytosine and 5-hydroxymethylcytosine.

### Nucleic Acid Loop

As used herein, the term “loop” in the polynucleotide refers to a single stranded stretch of one or more nucleotides, such as 2, 3, 4, 5, 6, 7, 8, 9, or 10 nucleotides, wherein the most 5′ nucleotide and the most 3′ nucleotide of the loop are each linked to a base-paired nucleotide in a stem.

### Nucleic Acid Stem

As used herein, the term “stem” refers to two or more base pairs, such as 3, 4, 5, 6, 7, 8, 9, 10, 11, 12, 13, 14, 15, 16, 17, 18, 19, 20 or more base pairs, formed by inverted repeat sequences connected at a “tip,” where the more 5′ or “upstream” strand of the stem bends to allows the more 3′ or “downstream” strand to base-pair with the upstream strand. The number of base pairs in a stem is the “length” of the stem. The tip of the stem is typically at least 3 nucleotides, but can be 4, 5, 6, 7, 8, 9, 10, 11, 12, 13, 14, 15 or more nucleotides. Larger tips with more than 5 nucleotides are also referred to as a “loop.” An otherwise continuous stem may be interrupted by one or more bulges as defined herein. The number of unpaired nucleotides in the bulge(s) are not included in the length of the stem. The position of a bulge closest to the tip can be described by the number of base pairs between the bulge and the tip (e.g., the bulge is 4 bps from the tip). The position of the other bulges (if any) further away from the tip can be described by the number of base pairs in the stem between the bulge in question and the tip, excluding any unpaired bases of other bulges in between.

### Nitro

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

### Non-Cationic Lipid

As used herein “non-cationic lipid” refers to any neutral, zwitterionic or anionic lipid.

### Open Reading Frame

An “open reading frame” is a continuous stretch of DNA beginning with a start codon (e.g., methionine (ATG)), and ending with a stop codon (e.g., TAA, TAG or TGA) and encodes a polypeptide.

### Orthologs

As used herein, “orthologs” refers to genes in different species that evolved from a common ancestral gene by speciation. Normally, orthologs retain the same function in the course of evolution. Identification of orthologs is critical for reliable prediction of gene function in newly sequenced genomes.

pegRNA

As used herein, the terms “prime editing guide RNA” or “pegRNA” or “PEgRNA” or “extended guide RNA” refer to a specialized form of a guide RNA that has been modified to include one or more additional sequences for implementing the prime editing methods and compositions described herein. As described herein, the prime editing guide RNA comprise one or more “extended regions” of nucleic acid sequence. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toeloop), or an RNA-protein recruitment domain (e.g., MS2 hairpin). As used herein the “primer binding site” comprises a sequence that hybridizes to a single-strand DNA sequence having a 3′ end generated from the nicked DNA of the R-loop.

### Pharmaceutically Acceptable Salt

The term “pharmaceutically acceptable salt” refers to a relatively non-toxic, inorganic or organic acid addition salt of a compound of the present disclosure which salt possesses the desired pharmacological activity.

### Pharmaceutically Acceptable Carrier, Diluent, or Excipient

As used herein, the term “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Various aspects and embodiments are described in further detail in the following subsections.

### Pharmaceutical Composition

As used herein the term “pharmaceutical composition” refers to compositions comprising at least one active ingredient (e.g., an LNP encapsulated with a mRNA payload) and optionally one or more pharmaceutically acceptable excipients.

### PEG

As used herein “PEG” means any polyethylene glycol or other polyalkylene ether polymer.

### Peptide

As used herein, “peptide” is less than or equal to 50 amino acids long, e.g., about 5, 10, 15, 20, 25, 30, 35, 40, 45, or 50 amino acids long.

### PolyA Tail

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

### Polyamine

As used herein, “polyamine” means compounds having two or more amino groups. Examples include putrescine, cadaverine, spermidine, and spermine.

### Polypeptide Variant

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants possess at least 50% identity to a native or reference sequence. In some embodiments, variants share at least 80%, or at least 90% identity with a native or reference sequence.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity.

### Protein Fragment, Function Protein Domains, Homologous Proteins

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided or referenced herein.

### Retron Gene Editing System

As used herein the term “retron gene editing system” refers to a set of components capable of conducting nucleic acid programmable gene editing that minimally comprises a programmable nuclease (e.g., a CRISPR type V nuclease, CRISPR type II nuclease, or a TnpB, and homologs and variants thereof) and a guide RNA which complexes with said nuclease. The guide RNA is capable of complexing with the programmable nuclease and directing the complex to a target DNA site that is complementary to the spacer sequence of the guide. A retron gene editing system also minimally comprises a retron non-coding RNA (retron ncRNA) comprising a template donor insert and a retron reverse transcriptase which converts the template donor insert into a single strand DNA template, which contains the desired edit. In certain embodiments, the ncRNA and the guide RNA are incorporated into a singular RNA molecule. In other embodiments, the ncRNA and guide RNA are provided in trans to one another. This term also embraces systems comprising additional accessory proteins, such as another nuclease, reverse transcriptase, deaminase, recombinase, transposase, integrase, DNA binding protein, transcription factor, a CRISPR Cas protein, or an anti-CRISPR inhibitor protein (Hwang and Maxwell, The CRISPR Journal; Volume 2, Number 1, 2019, which is incorporated by reference herein in its entirety). In various embodiments, the gene editing system components, such as the nuclease, guide RNAs, ncRNAs, or RTs or accessory proteins, may comprise one or more NLSs to facilitate their transfer into the nucleus. The gene editing system may be in the form of DNA (encoding the protein components and the guide RNA and the ncRNA), RNA (encoding the protein components and constituting a guide RNA and a ncRNA), or a mixture of protein components (e.g., a nuclease) and RNA (e.g., a guide RNA or ncRNA).

### RNA

The term “RNA” is a well-known term of art that refers to ribonucleic acid.

### RNA-Guided Nuclease

As used herein, an “RNA-guided nuclease” is a type of “programmable nuclease,” and a specific type of “nucleic acid-guided nuclease.” As used herein, the term “RNA-guided nuclease” or “RNA-guided endonuclease” refers to a nuclease that associates covalently or non-covalently with a guide RNA thereby forming a complex between the guide RNA and the RNA-guided nuclease. The guide RNA comprises a spacer sequence which comprises a nucleotide sequence having complementarity with a strand of a target DNA sequence. Thus, the RNA-guided nuclease is indirectly guided or programmed to localize to a specific site in a DNA molecule through its association with the guide RNA, which directly binds or anneals to a strand of the target DNA through its complementarity region via Watson-Crick base-pairing.

### Sequence Identity

As used herein, the term “sequence identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). For example, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna. CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990).

### Spacer

As used herein the term “spacer” refers to a region of a polynucleotide or polypeptide ranging from 1 residue to hundreds or thousands of residues separating two other elements in a sequence. The sequence of the spacer can be defined or random. A spacer sequence is typically non-coding but may be a coding sequence.

### Stem and Loop

As used herein, the term “stem” refers to two or more base pairs, such as 3, 4, 5, 6, 7, 8, 9, 10, 11, 12, 13, 14, 15, 16, 17, 18, 19, 20 or more base pairs, formed by inverted repeat sequences connected at a “tip,” where the more 5′ or “upstream” strand of the stem bends to allows the more 3′ or “downstream” strand to base-pair with the upstream strand. The number of base pairs in a stem is the “length” of the stem. The tip of the stem is typically at least 3 nucleotides, but can be 4, 5, 6, 7, 8, 9, 10, 11, 12, 13, 14, 15 or more nucleotides.

Larger tips with more than 5 nucleotides are also referred to as a “loop.” An otherwise continuous stem may be interrupted by one or more bulges as defined herein. The number of unpaired nucleotides in the bulge(s) are not included in the length of the stem. The position of a bulge closest to the tip can be described by the number of base pairs between the bulge and the tip (e.g., the bulge is 4 bps from the tip). The position of the other bulges (if any) further away from the tip can be described by the number of base pairs in the stem between the bulge in question and the tip, excluding any unpaired bases of other bulges in between. As used herein, the term “loop” in the polynucleotide refers to a single stranded stretch of one or more nucleotides, such as 2, 3, 4, 5, 6, 7, 8, 9, or 10 nucleotides, wherein the most 5′ nucleotide and the most 3′ nucleotide of the loop are each linked to a base-paired nucleotide in a stem.

A “stem-loop structure” or a “hairpin” refers to a nucleic acid having a secondary structure that includes a region of nucleotides which are known or predicted to form a double strand (stem portion) that is linked on one side by a region of predominantly single-stranded nucleotides (loop portion). Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e., not include any mismatches

### Specific Delivery

As used herein, the term “specific delivery” to a particular class of cells, an organ, or a system or group thereof implies that a higher proportion of lipid nanoparticles including a therapeutic and/or prophylactic (e.g., a gene editing system defined herein) are delivered to the destination (e.g., tissue or cell) of interest relative to other destinations upon administration of an LNP to a mammal. In some embodiments, specific delivery may result in a greater than 2-fold, 5-fold, 10-fold, 15-fold, or 20-fold increase in the amount of therapeutic and/or prophylactic per 1 g of tissue of the targeted destination (e.g., tissue of interest, such as a bone marrow) as compared to another destination (e.g., the liver).

### Subject

As used herein, the term “subject” refers to an individual organism, for example, an individual mammal or plant. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. The terms “individual,” “subject,” “host,” and “patient,” used interchangeably herein.

### Substituted

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, for example, 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

As described herein, compounds of the present disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; C(O)C(O)R^{∘}; —C(O)CH₂C(O)R^{∘}; —C(NOR)R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene) C(O)O—N(R^{∘})₂, wherein each R^{∘} may be substituted as defined below and is independently hydrogen, C1-6 aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{•} include ═O and ═S.

Suitable divalent substituents on a saturated carbon atom of an “optionally substituted” group include the following: ═O, ═S, ═NNR*2, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, C(O)R^{†}, —C(O)OR^{†}, —C(O)C(O)R^{†}, —C(O)CH₂C(O)R^{†}, —S(O)₂R^{†}, —S(O)₂NR^{†}₂, C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of RT, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation, for example, by rearrangement, cyclization, or elimination.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

### Structural Lipid

As used herein “structural lipid” refers to sterols and lipids containing sterol moieties.

### Substantially

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### Target or Target Nucleic Acid

A “target” or “target nucleic acid” as used herein is a polynucleotide (e.g., DNA such as genomic DNA) that includes a site (“target site” or “target sequence”) targeted by a nucleic acid programmable DNA binding protein of the present disclosure. The target sequence is the sequence to which the guide sequence of a subject nucleic acid programmable DNA binding protein will hybridize. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.”

### Terminus

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH₂)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

### Treating

The terms “treat,” “treating,” and “treatment,” as used herein, refer to therapeutic or preventative measures such as those described herein.

### Unmodified

As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Upstream and Downstream

As used herein, the terms “upstream” and “downstream” are terms of relativity that define the linear position of at least two elements located in a nucleic acid molecule (whether single or double-stranded) that is orientated in a 5′-to-3′ direction. A first element is said to be upstream of a second element in a nucleic acid molecule where the first element is positioned somewhere that is 5′ to the second element. Conversely, a first element is downstream of a second element in a nucleic acid molecule where the first element is positioned somewhere that is 3′ to the second element.

### Vaccine

As used herein, the phrase “vaccine” refers to a biological preparation that improves immunity in the context of a particular disease, disorder or condition.

### Vector

As used herein, a “vector” is any molecule or moiety which transports, transduces or otherwise acts as a carrier of a heterologous molecule. Vectors of the present disclosure may be produced recombinantly and may be based on and/or may comprise viral parent or reference sequence. Such parent or reference viral sequences may serve as an original, second, third or subsequent sequence for engineering vectors. In non-limiting examples, such parent or reference viral sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These viral sequences may serve as either the “donor” sequence of one or more codons (at the nucleic acid level) or amino acids (at the polypeptide level) or “acceptor” sequences of one or more codons (at the nucleic acid level) or amino acids (at the polypeptide level). The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### 3′ Untranslated Region

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

### 5′ Untranslated Region

A “5′ untranslated region” (UTR) refers to a region of an mRNA that is directly upstream (i.e., 5′) from the start codon (i.e., the first codon of an mRNA transcript translated by a ribosome) that does not encode a polypeptide.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### Abbreviations and Initialisms

As used herein, the following abbreviations and initialisms have the indicated meanings:

| | |
|---|---|
| MC3 | 4-(dimethylamino)-butanoic acid, (10Z,13Z)-1-(9Z,12Z)-9,12-octadecadien-1-yl- |
| | 10,13-nonadecadien-1-yl ester |
| DSPC | 1,2-distearoyl-sn-glycero-3-phosphocholine |
| DMG | 1,2-Dimyristoyl-rac-glycero-3-methanol |
| DOMG-PEG | R-3-[(ω-methoxy-poly(ethyleneglycol))carbamoyl)]-1,2-dimyristyloxypropyl-3-amine |
| DLPE | 1,2-Dilauroyl-sn-Glycero-3-Phosphoethanolamine |
| DMPE | 1,2-Dimyristoyl-sn-Glycero-3-Phosphoethanolamine |
| DPPC | 1,2-dipalmitoyl-sn-glycero-3-phosphocholine |
| DSPE | 1,2-distearoyl-sn-glycero-3-phosphoethanolamine |
| DDAB | Didodecyldimethylammonium bromide |
| EPC | 1,2-dioleoyl-sn-glycero-3-ethylphosphocholine |
| 14PA | 1,2-dimyristoyl-sn-glycero-3-phosphate |
| 18BMP | bis(monooleoylglycero)phosphate |
| DODAP | 1,2-dioleoyl-3-dimethylammonium-propane |
| DOTAP | 1,2-dioleoyl-3-trimethylammonium-propane |
| C12-200 | 1,1′-((2-(4-(2-((2-(bis(2-hydroxydodecyl)amino)ethyl)(2- |
| | hydroxydodecyl)amino)ethyl)piperazin-1-yl)ethyl)azanediyl)bis(dodecan-2-ol) |
| MLP | Multi-Lymphoid Progenitor |
| CMP | Common Myeloid Progenitor |
| GMP | Granulocyte/Macrophage Progenitor |
| MEP | Megakaryocyte/Erythroid Progenitor |
| HSC | Hematopoietic Stem Cells |
| MPP | Multipotent Progenitor |
| CLP | Common Lymphoid Progenitor |
| LSK | Hematopoietic Stem and Progenitor Cells |
| HSC | Hematopoietic Stem Cell |
| LT-HSC | Long Term Hematopoietic Stem Cell |
| ST-IISC | Short Term Hematopoietic Stem Cell |
| TDT | Transfusion-dependent β-thalassemia |
| SCD | Sickle-cell disease |
| HBB | Refers to hemoglobin subunit beta or gene encoding same |
| HBA1 | Refers to hemoglobin subunit alpha 1 or gene encoding same |
| HBA2 | Refers to hemoglobin subunit alpha 2 or gene encoding same |
| HBG1 | Refers to hemoglobin subunit gamma 1 or gene encoding same |
| HBG2 | Refers to hemoglobin subunit gamma 2 or gene encoding same |
| HBD | Refers to hemoglobin subunit delta or gene encoding same |
| HBE | Refers to hemoglobin subunit epsilon or gene encoding same |
| HBG1/2 | Refers to the pair of gene promoters for the genes encoding hemoglobin subunit |
| promoter | gamma 1 and hemoglobin subunit gamma 2. The HIBG1/2 promoters have the same |
| | sequence. |
| LCR | Refers to the betaglobin locus control region, which functions in the regulation of the |
| | expression of hemoglobin subunit genes, including HBB and HBG1/2 genes |
| BE | Base editing |
| PE | Prime editing |
| pegRNA | Prime editing guide RNA |
| HDR | Homology-directed repair |
| HPFH | Refers to a set of mutations in the human population which are associated with |
| | hereditary persistence of fetal hemoglobin |

### Equivalents and Scope

Those skilled in the art will recognize, or be able to ascertain using no more than routine experimentation, many equivalents to the specific embodiments in accordance with the present disclosure. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

### Sequences

The following sequences form part of the specification, each of which are part of the instant Specification and can be found in Appendix A (which itself is part of the Specification) of the Sequence Listing which accompanies and forms part of this disclosure as originally filed.

| SEQ ID NO: | Description |
|---|---|
| 1-30 | Exemplary 5′ UT sequences - Table Z |
| 31-32 | Exemplary Stop Elements - Table Y |
| 33-37 | Exemplary linkers |
| 38-46 | Exemplary Nuclear Localization Domains |
| 47 | Cre Recombinase mRNA |
| 48 | VHH mRNA |
| 49-50 | BCL11a targeting gRNA and Cas9 mRNA |
| 51 | Exemplary modified gRNA sequence |
| 52-80 | Type V proteins and arginine scanning mutagenesis and |
| | primers - Example 13 |
| 81-93 | NLS and linker sequences - Example 14 |
| 96-126 | Directed Evolution of Type V editor (AA Sequences) - |
| | Example 15 |
| 127-157 | Directed Evolution of Type V editor (Nucleotide |
| | Sequences) - Example 15 |
| 158-191 | Primer Sequences used in Directed Evolution of Type V |
| | editor |
| 192 | gRNA direct repeat sequence - Example 16 |
| 193-213 | Exemplary Modified PCSK9, B2M and Edit 301 guide |
| | sequences - Example 16 |
| 214-218 | Exemplary modified guide sequences |
| 219 | Exemplary portion of PNA-NLS probe - Example 18 |
| 220-537 | Exemplary gRNA sequences |
| 538-679 | Exemplary spacer sequences |
| 680-683 | Exemplary modified guide RNAs - Example 19 |
| 684-720 | Exemplary modified guide RNAs - Example 19 |
| 721-735 | Exemplary miRNA sequences - Example 20 |
| 736-750 | Exemplary miRNA Target Sequences - Example 20 |
| 751-753 | Exemplary gRNA targeting TTR - Example 18 |
| 754-792 | Directed Evolution of Type V editor (AA Sequences) - |
| | Example 15 - continued |
| 793-831 | Directed Evolution of Type V editor (Nucleotide |
| | Sequences) - Example 15 - continued |

### Numbered Paragraphs

Without limitation, the following numbered paragraphs (or embodiments) are contemplated embodiments within the scope of the instant specification.
1. A pharmaceutical composition comprising:
   a) at least one lipid nanoparticle (LNP) comprising at least one ionizable lipid selected from those listed in Tables (I), (II), (III), (IV) and (V); and
   b) at least one gene editing system capable of editing, modifying or altering a polynucleotide sequence that results in treatment of a hemoglobinopathy.
   —or—
1. A pharmaceutical composition comprising:
   a) at least one lipid nanoparticle (LNP) comprising at least one ionizable lipid selected from those listed in Tables (I), (II), (III), (IV), (V), (VI), (VII), (VIII), (IX), and (X); and
   b) at least one gene editing system capable of editing, modifying or altering a polynucleotide sequence that results in treatment of a hemoglobinopathy.
2. The pharmaceutical composition of embodiment 1, wherein the at least one gene editing system comprises a gene editing system that corrects a sickle cell mutation by repairing the β-globin gene.
3. The pharmaceutical composition of any one of embodiments 1-2, wherein the at least one gene editing system comprises a gene editing system that corrects a sickle cell mutation through homology directed repair.
4. The pharmaceutical composition of any one of embodiments 1-2, wherein the at least one gene editing system comprises a gene editing system that corrects a sickle cell mutation through non-homologous end-joining (NHEJ), targeting aberrant splicing sites in introns I and II, resulting in restoration of functional β-globin transcripts.
5. The pharmaceutical composition of any one of embodiments 1-2, wherein the at least one gene editing system comprises a gene editing system that corrects a sickle cell mutation through a single base pair gene correction.
6. The pharmaceutical composition of any one of embodiments 1-2, wherein the at least one gene editing system comprises a gene editing system that corrects a sickle cell mutation by executing a single base pair correction, converting the aberrant thymine to adenine or cytosine.
7. The pharmaceutical composition of claim 1, wherein the at least one gene editing system comprises a gene editing system that reactivates γ-globin production.
8. The pharmaceutical composition of any one of embodiments 1 and 7, wherein the at least one gene editing system comprises a gene editing system that eliminates an HbF inhibitory sequence, or a portion thereof.
9. The pharmaceutical composition of any one of embodiments 1 and 7, wherein the at least one gene editing system comprises a gene editing system that targets HBG1 and/or HBG2 promoters, resulting in disruption of binding sites for γ-globin transcriptional repressors.
10. The pharmaceutical composition of any one of embodiments 1 and 7, wherein the at least one gene editing system comprises a gene editing system that disrupts BCL11A binding sites.
11. The pharmaceutical composition of any one of embodiments 1 and 7, wherein the at least one gene editing system comprises a gene editing system that disrupts the GATA1 motif in the erythroid-specific intronic enhancer of BCL11A.
12. The pharmaceutical composition of any one of the previous embodiments, wherein the at least one gene editing system comprises a gene editing system, or components thereof, disclosed in any one of:

US Patents

| | | | | | |
|---|---|---|---|---|---|
| 11,242,525 | 11,572,543 | 11,268,077 | 11,466,271 | 9,957,501 | 10,808,020 |
| 11,021,696 | 10,563,184 | 11,155,803 | 10,738,305 | 11,344,609 | 10,435,677 |
| 11,466,271 | | | | | |

US Patent Application Publications

| | | | |
|---|---|---|---|
| US20200339979A1 | US20210180091A1 | US20200140896A1 | US20190010495A1 |
| US20210254061A1 | US20210040506A1 | US20230002760A1 | US20230018543A1 |
| US20180273609A1 | US20220047637A1 | US20190184035A1 | US20200255857A1 |
| US20200399626A1 | US20220228142A1 | US20180200387A1 | US20180030438A1 |
| US20220401530A1 | US20210371858A1 | US20190010495A1 | US20210180091A1 |
| US20210008161A1 | US20210155927A1 | US20200330609A1 | US20200384033A1 |

PCT Publications

| | | | |
|---|---|---|---|
| WO2022240787A1 | WO2021119040A1 | WO2023014839A1 | WO2023039440A2 |
| WO2022120094A2 | WO2022155458A1 | WO2023019227A1 | WO2023019226A1 |
| WO2022067122A1 | WO2023019225A2 | WO2021163587A1 | WO2024020346A1 |
| WO2017191503A1 | WO2021144692A1 | | |

13. The pharmaceutical composition of any one of the preceding embodiments, wherein the gene editing system comprises a CRISPR-Cas gene editing system or components thereof, or a polynucleotide encoding the same.
14. The pharmaceutical composition of any one of the preceding embodiments, wherein the gene editing system comprises a Cas9 nuclease, or a polynucleotide encoding the same.
15. The pharmaceutical composition of any one of the preceding embodiments, wherein the gene editing system comprises a Cas12a nuclease, or a polynucleotide encoding the same.
16. The pharmaceutical composition of any one of the preceding embodiments, wherein the gene editing system comprises a prime editing system or components thereof, or a polynucleotide encoding the same.
17. The pharmaceutical composition of any one of the preceding embodiments, wherein the gene editing system comprises a retron editing system or components thereof, or a polynucleotide encoding the same.
18. The pharmaceutical composition of any one of the preceding embodiments, wherein the gene editing system comprises a TnpB editing system or components thereof, or a polynucleotide encoding the same.
19. The pharmaceutical composition of any one of the preceding embodiments, wherein the gene editing system comprises an integrase editing system or components thereof, or a polynucleotide encoding the same.
20. The pharmaceutical composition of any one of the preceding embodiments, wherein the gene editing system comprises a base editing system or components thereof, or a polynucleotide encoding the same.
21. The pharmaceutical composition of any one of the preceding embodiments, wherein the gene editing system comprises an epigenetic editing system or components thereof, or a polynucleotide encoding the same.
22. The pharmaceutical composition of any one of the preceding embodiments, wherein the gene editing system comprises a gene writing system or components thereof, or a polynucleotide encoding the same.
23. The pharmaceutical composition of any one of the preceding embodiments, wherein the gene editing system comprises a gene inactivating system or components thereof, or a polynucleotide encoding the same.
24. The pharmaceutical composition of any one of the preceding embodiments, wherein the gene editing system comprises zinc finger nuclease or components thereof, or a polynucleotide encoding the same.
25. The pharmaceutical composition of any one of the preceding embodiments, wherein the gene editing system comprises a TALE Nuclease, a TALE nickase, Zinc Finger (ZF) Nuclease, ZF Nickase, meganuclease, or a combination thereof, or components thereof, or a polynucleotide encoding any of the same.
26. The pharmaceutical composition of any one of the preceding embodiments, wherein the gene editing system comprises a meganuclease, or a polynucleotide encoding the same.
27. The pharmaceutical composition of any one of the preceding embodiments, wherein the gene editing system comprises at least one guide RNA (gRNA).
28. The pharmaceutical composition of any one of the preceding embodiments, wherein the gene editing system comprises at least one guide RNA, or a portion thereof, disclosed in any one of:

US Patents

| | | | | | |
|---|---|---|---|---|---|
| 11,242,525 | 11,572,543 | 11,268,077 | 11,466,271 | 9,957,501 | 10,808,020 |
| 11,021,696 | 10,563,184 | 11,155,803 | 10,738,305 | 11,344,609 | 10,435,677 |
| 11,466,271 | | | | | |

US Patent Application Publications

| | | | |
|---|---|---|---|
| US20200339979A1 | US20210180091A1 | US20200140896A1 | US20190010495A1 |
| US20210254061A1 | US20210040506A1 | US20230002760A1 | US20230018543A1 |
| US20180273609A1 | US20220047637A1 | US20190184035A1 | US20200255857A1 |
| US20200399626A1 | US20220228142A1 | US20180200387A1 | US20180030438A1 |
| US20220401530A1 | US20210371858A1 | US20190010495A1 | US20210180091A1 |
| US20210008161A1 | US20210155927A1 | US20200330609A1 | US20200384033A1 |

PCT Publications

| | | | |
|---|---|---|---|
| WO2022240787A1 | WO2021119040A1 | WO2023014839A1 | WO2023039440A2 |
| WO2022120094A2 | WO2022155458A1 | WO2023019227A1 | WO2023019226A1 |
| WO2022067122A1 | WO2023019225A2 | WO2021163587A1 | WO2024020346A1 |
| WO2017191503A1 | WO2021144692A1 | | |

29. The pharmaceutical composition of any one of the preceding embodiments, wherein the at least one lipid nanoparticle further comprises:
i) at least one structural lipid;
ii) at least one phospholipid; and
iii) at least one PEGylated lipid.
30. The pharmaceutical composition of any one of the preceding embodiments, wherein the at least one structural lipid is selected from cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and any combinations thereof.
31. The pharmaceutical composition of any one of the preceding embodiments, wherein the at least one phospholipid is selected from 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium(S)-2-ammonio-3-((((R)-2-(oleoyloxy)-3-(stearoyloxy) propoxy) oxidophosphoryl)oxy) propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), diolcoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl)phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin.
32. The pharmaceutical composition of any one of the preceding embodiments, wherein the at least one PEGylated lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-CDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N (Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.
33. The pharmaceutical composition of any one of the preceding embodiments, wherein the LNP further comprises at least one additional lipid component selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Na-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-α-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g. 1,2-dioleoyl-sn-glycero-3-phosphoethanolamine-N-[4-(p-maleimidophenyl) but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol (PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoylolcoylphosphatidylcholine (POPC), phosphatidylethanolamine (POPE), palmitoyloleyolphosphatidylglycerol (POPG), phosphatidylserine (PS), lissamine rhodamineB-phosphatidylethanolamine lipid (Rh-PE), purifiedsoy-derivedmixtureofphospholipids (SIOO), phosphatidylcholine (SM), 18-1-trans-PE, 1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.
34. The pharmaceutical composition of any one of the preceding embodiments, wherein the LNP further comprises one or more targeting moieties.
35. A method of treating a hemoglobinopathy in a patient in need thereof, the method comprising administering to the subject a pharmaceutical composition of any one of the preceding embodiments.
36. The pharmaceutical composition of any of the preceding embodiments for use as a medicament in the treatment of a hemoglobinopathy.
37. Use of a pharmaceutical composition of any of the preceding embodiments for the manufacture of a medicament for delivery of a gene editing system capable of treating a hemoglobinopathy.
38. The pharmaceutical composition of any one of the preceding embodiments, wherein the lipid nanoparticle comprises:
   (a) a PEG lipid selected from PEG2k-DMG or PEG2k-DSPE or a mixture thereof;
   (b) cholesterol;
   (c) a phospholipid selected from sphingolipid or DSPC or a mixture thereof.
39. The pharmaceutical composition of any one of the preceding embodiments, wherein the lipid nanoparticle comprises:
   (a) about 0 mol % to about 10 mol % of PEG lipid;
   (b) about 0 mol % to about 30 mol % structural lipid;
   (c) about 20 mol % to about 45 mol % phospholipid; and
   (d) about 30 mol % to about 60 mol % of at least one ionizable lipid selected from those listed in Tables (I), (II), (III), (IV), (V), (VI), (VII), (VIII), (IX), and (X).
40. The pharmaceutical composition of any one of the preceding embodiments, wherein the lipid nanoparticle comprises:
   (a) about 1 mol % to about 2 mol % of PEG lipid;
   (b) about 25 mol % to about 40 mol % structural lipid;
   (c) about 20 mol % to about 45 mol % phospholipid; and
   (d) about 30 mol % to about 60 mol % of at least one ionizable lipid selected from those listed in Tables (I), (II), (III), (IV), (V), (VI), (VII), (VIII), (IX), and (X).
41. The pharmaceutical composition of any one of the preceding embodiments, wherein the lipid nanoparticle comprises:
   (a) about 2 mol % of PEG lipid;
   (b) about 25 mol % structural lipid;
   (c) about 40 mol % phospholipid; and
   (d) about 33 mol % of at least one ionizable lipid selected from those listed in Tables (I), (II), (III), (IV), (V), (VI), (VII), (VIII), (IX), and (X).
42. The pharmaceutical composition of any one of the preceding embodiments, wherein the lipid nanoparticle comprises:
   (a) about 2.5 mol % of PEG lipid;
   (b) about 39 mol % structural lipid;
   (c) about 10 mol % phospholipid; and
   (d) about 48.5 mol % of at least one ionizable lipid selected from those listed in Tables (I), (II), (III), (IV), (V), (VI), (VII), (VIII), (IX), and (X).
43. The pharmaceutical composition of any one of the preceding embodiments, wherein the lipid nanoparticle comprises:
   (a) about 1.5 mol % of PEG lipid;
   (b) about 40 mol % structural lipid;
   (c) about 10 mol % phospholipid; and
   (d) about 48.5 mol % of at least one ionizable lipid selected from those listed in Tables (I), (II), (III), (IV), (V), (VI), (VII), (VIII), (IX), and (X).
44. The pharmaceutical composition of any one of the preceding embodiments, wherein the lipid nanoparticle comprises an mRNA or a circRNA encoding a CRISPR nuclease and a guide RNA capable of enacting a genetic edit that disrupts the GATA1 motif in the erythroid-specific intronic enhancer of BCL11A.
45. The pharmaceutical composition of any one of the preceding embodiments, wherein the lipid nanoparticle comprises an mRNA or a circRNA encoding a CRISPR nuclease and a guide RNA capable of enacting a genetic edit that disrupts BCL11A binding sites.

Without limitation, the present disclosure further contemplates the following numbered paragraphs (or embodiments) within the scope of the instant specification.
1. A lipid nanoparticle comprising:
a) a cargo comprising one or more nucleic acid molecules encoding and/or constituting a gene editing system capable of installing an edit in a hemoglobin gene or regulatory region thereof;
b one or more ionizable lipids;
c) one or more phospholipids, in an amount of about 20 mol % to about 60 mol % of the total lipid content of the lipid nanoparticle;
d) one or more structural lipids; and
e) one or more PEG lipids.
2. The lipid nanoparticle of embodiment 1, wherein the lipid nanoparticle does not comprise a targeting moiety.
3. The lipid nanoparticle of any one of embodiments 1-2, wherein the one or more nucleic acid molecules are DNA and/or RNA molecules.
4. The lipid nanoparticle of any one of embodiments 1-3, wherein the one or more nucleic acid molecules are RNA molecules.
5. The lipid nanoparticle of any one of embodiments 1-4, wherein the gene editing system comprises (i) a zinc finger protein, (ii) a TALEN protein, or (iii) a nucleic acid programmable nuclease.
6. The lipid nanoparticle of any one of embodiments 1-4, wherein the gene editing system is a CRISPR-type II gene editing system, a CRISPR-type V gene editing system, or a retron gene editing system.
7. The lipid nanoparticle of embodiment 6, wherein the CRISPR-type II gene editing system comprises a CRISPR-type II nuclease or functional variant or ortholog thereof and a guide RNA.
8. The lipid nanoparticle of embodiment 6, wherein the CRISPR-type V gene editing system comprises a CRISPR-type V nuclease or functional variant or ortholog thereof and a guide RNA.
9. The lipid nanoparticle of embodiment 6, wherein the retron gene editing system comprises a CRISPR-type II or type V nuclease or functional variant or ortholog thereof, a retron non-coding RNA (ncRNA), and a guide RNA.
10. The lipid nanoparticle of any one of embodiments 1-4, wherein the gene editing system is a base editor comprising a CRISPR-type II or type V nuclease or functional variant or ortholog thereof, a deaminase or functional variant or ortholog thereof, and a guide RNA.
11. The lipid nanoparticle of any one of embodiments 1-4, wherein the gene editing system is a prime editor comprising a CRISPR-type II or type V nuclease or functional variant or ortholog thereof, a reverse transcriptase or functional variant or ortholog thereof, and a prime editing guide RNA (pegRNA).
12. The lipid nanoparticle of any one of embodiments 7-11, wherein the guide RNA or pegRNA comprises a spacer sequence that is complementary to a target sequence.
13. The lipid nanoparticle of embodiment 12, wherein the target sequence is a hemoglobin gene or regulatory region thereof.
14. The lipid nanoparticle of embodiment 13, wherein the hemoglobin gene or regulatory region thereof is the gene encoding hemoglobin beta subunit.
15. The lipid nanoparticle of embodiment 13, wherein the hemoglobin gene or regulatory region thereof is the BCL11A binding site in the HBG1/2 promoter.
16. The lipid nanoparticle of embodiment 13, wherein the hemoglobin gene or regulatory region thereof is the enhancer sequence of the gene encoding BCL11A.
17. The lipid nanoparticle of embodiment 13, wherein the hemoglobin gene or regulatory region thereof is the binding site of the GATA-1 activator in the HBG1/2 promoter.
18. The lipid nanoparticle of any one of embodiments 6 and 8-17, wherein the guide RNA comprises a sequence selected from:
   (a) SEQ ID NO: 51;
   (b) SEQ ID NOs: 207-218;
   (c) SEQ ID NOs: 220-537; and
   (d) SEQ ID NOs: 680-720;
      or any portion thereof or a sequence having at least 80%, or 85% or 90%, or 95%, or 99%, or up to 100% sequence identity with any of the aforementioned sequences.
19. The lipid nanoparticle of any one of embodiments 6 and 8-17, wherein the guide RNA comprises miRNA sequences selected from:
   (a) SEQ ID NO: 721-735;
   (b) SEQ ID NOs: 721; and
   (c) SEQ ID NOs: 723;
      or any portion thereof or a sequence having at least 80%, or 85% or 90%, or 95%, or 99%, or up to 100% sequence identity with any of the aforementioned sequences.
20. The lipid nanoparticle of any one of embodiments 6 and 8-17, wherein the CRISPR-type V nuclease comprises SEQ ID NOs: 52, 53, 57-80, 84-93, 96-126, 754-792 or any amino acid sequence having at least 80%, or 85% or 90%, or 95%, or 99%, or up to 100% sequence identity with any of the aforementioned sequences.
21. The lipid nanoparticle of any one of embodiments 1-20, wherein the gene editing system comprises one or more additional accessory proteins.
22. The lipid nanoparticle of embodiment 21, wherein the one or more accessory proteins comprises a nuclease, reverse transcriptase, recombinase, transposase, integrase, DNA binding protein, transcription factor, a CRISPR Cas protein, or an anti-CRISPR inhibitor protein.
23. The lipid nanoparticle of embodiment 22, wherein the CRISPR Cas protein is Cas1, Cas2, Cas3, Cas4, Cas5, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas13, Cas13a (C2c2), Cas13b, Cas13c, Cas13d, Cas13x.1, Csc1, Csc2, Csy1, Csy2, Csy3, Csm2, Cmr5, Csx10, Csf1, Csn2, C2c4, C2c8, or C2c9.
24. The lipid nanoparticle of any one of embodiments 21-23, wherein the one or more accessory proteins may be provided in trans or may be coupled to one or more components of the gene editing system.
25. The lipid nanoparticle of any one of embodiments 21-23, wherein the one or more accessory proteins is coupled to one or more components of the gene editing system by a linker.
26. The lipid nanoparticle of embodiment 25, wherein the linker has an amino acid sequence of any one of SEQ ID NOs: 33-37 and 81-83.
27. The lipid nanoparticle of any one of embodiments 1-26, wherein the N:P ratio is from about 5:1 to about 8:1.
28. The lipid nanoparticle of any one of embodiments 1-27, wherein the lipid nanoparticle comprises about 25 mol % to about 45 mol % of the one or more ionizable lipids, as a proportion of the total lipid content of the lipid nanoparticle.
29. The lipid nanoparticle of any one of embodiments 1-28, wherein the lipid nanoparticle comprises about 15 mol % to about 35 mol % of the one or more structural lipids, as a proportion of the total lipid content of the lipid nanoparticle.
30. The lipid nanoparticle of any one of embodiments 1-29, wherein the lipid nanoparticle comprises about 1 mol % to about 3 mol % of the one or more PEG lipids, as a proportion of the total lipid content of the lipid nanoparticle.
31. The lipid nanoparticle of any one of embodiments 1-27, wherein the lipid nanoparticle comprises (a) about 1 mol % to about 3 mol % of the one or more PEG lipids; (b) about 15 mol % to about 35 mol % of the one or more structural lipids; (c) about 30 mol % to about 60 mol % of the one or more phospholipids; and (d) about 25 mol % to about 45 mol % of the one or more ionizable lipids.
32. The lipid nanoparticle of any one of embodiments 1-27, wherein the lipid nanoparticle comprises (a) about 1.5 mol % to about 2.5 mol % of the one or more PEG lipids; (b) about 20 mol % to about 30 mol % of the one or more structural lipids; (c) about 35 mol % to about 45 mol % of the one or more phospholipids; and (d) about 28 mol % to about 40 mol % of the one or more ionizable lipids.
33. The lipid nanoparticle of any one of embodiments 1-32, wherein the ionizable lipid is selected from any one of the compounds described in Tables I-X.
34. The lipid nanoparticle of any one of embodiments 1-33, wherein the phospholipid is selected from 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium(S)-2-ammonio-3-((((R)-2-(oleoyloxy)-3-(stearoyloxy) propoxy) oxidophosphoryl)oxy) propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), diolcoylphosphatidylglycerol (DOPG), 1,2-diolcoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin, or combinations of 2, 3, 4, 5, 6, 7, 8, 9, 10, 11, or 12 of the above phospholipids.
35. The lipid nanoparticle of any one of embodiments 1-34, wherein the structural lipid is selected from cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and any combinations thereof.
36. The lipid nanoparticle of any one of embodiments 1-35, wherein the PEG lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-CDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N (Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X, and any combinations thereof.
37. The lipid nanoparticle of any one of embodiments 1-36, wherein the one or more phospholipids comprises one or more selected from phosphatidylcholine, phosphatidylserine, phosphoethanolamine, and sphingoid lipids or a combination thereof.
38. The lipid nanoparticle of any one of embodiments 1-37, wherein the one or more phospholipids comprises 1,2-distearoyl-sn-glycero-3-phosphocholine (DSPC), sphingomyelin or a combination thereof.
39. The lipid nanoparticle of any one of embodiments 1-38, wherein the one or more phospholipids comprises two or more phospholipids, such that no single phospholipid makes up more than 30 mol % of the total lipid content of the nanoparticle.
40. The lipid nanoparticle of any one of embodiments 1-38, wherein the lipid nanoparticle comprises about 40 mol % 1,2-distearoyl-sn-glycero-3-phosphocholine (DSPC).
41. The lipid nanoparticle of any one of embodiments 1-38, wherein the lipid nanoparticle comprises about 40 mol % sphingomyelin.
42. The lipid nanoparticle of any one of embodiments 1-38, wherein the phospholipid is sphingomyelin, the structural lipid is cholesterol, and the PEG lipid is DMG-PEG2k.
43. The lipid nanoparticle of any one of embodiments 1-38, wherein the ionizable lipid is any compound from Tables I-X, the phospholipid is sphingomyelin, the structural lipid is cholesterol, and the PEG lipid is DMG-PEG2k.
44. The lipid nanoparticle of any one of embodiments 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % sphingomyelin; and (d) about 25 mol % to about 45 mol % of the one or more ionizable lipids.
45. The lipid nanoparticle of any one of embodiments 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % sphingomyelin; and (d) about 25 mol % to about 45 mol % of an ionizable lipid selected from those in Tables I-X, or any combinations thereof.
46. The lipid nanoparticle of any one of embodiments 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % sphingomyelin; and (d) about 28 mol % to about 40 mol % of the one or more ionizable lipids.
47. The lipid nanoparticle of any one of embodiments 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % sphingomyelin; and (d) about 28 mol % to about 40 mol % of an ionizable lipid selected from those in Tables I-X, or any combinations thereof.
48. The lipid nanoparticle of any one of embodiments 1-38, wherein the lipid nanoparticle comprises (a) about 2 mol % of DMG-PEG2k; (b) about 25 mol % cholesterol; (c) about 40 mol % sphingomyelin; and (d) about 33 mol % of an ionizable lipid selected from those in Tables I-X, or any combinations thereof.
49. The lipid nanoparticle of any one of embodiments 1-38, wherein the lipid nanoparticle comprises (a) about 2 mol % of DMG-PEG2k; (b) about 25 mol % cholesterol; (c) about 40 mol % sphingomyelin; and (d) about 33 mol % of the one or more ionizable lipids.
50. The lipid nanoparticle of any one of embodiments 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % DSPC; and (d) about 25 mol % to about 45 mol % of the one or more ionizable lipids.
51. The lipid nanoparticle of any one of embodiments 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % DSPC; and (d) about 25 mol % to about 45 mol % of an ionizable lipid selected from those in Tables I-X, or any combinations thereof.
52. The lipid nanoparticle of any one of embodiments 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % DSPC; and (d) about 28 mol % to about 40 mol % of the one or more ionizable lipids.
53. The lipid nanoparticle of any one of embodiments 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % DSPC; and (d) about 28 mol % to about 40 mol % of an ionizable lipid selected from those in Tables I-X, or any combinations thereof.
54. The lipid nanoparticle of any one of embodiments 1-38, wherein the lipid nanoparticle comprises (a) about 2 mol % of DMG-PEG2k; (b) about 25 mol % cholesterol; (c) about 40 mol % DSPC; and (d) about 33 mol % of an ionizable lipid selected from those in Tables I-X, or any combinations thereof.
55. The lipid nanoparticle of any one of embodiments 1-38, wherein the one or more phospholipids comprises a mixture of sphingomyelin and DSPC, the structural lipid is cholesterol, and the PEG lipid is DMG-PEG2k.
56. The lipid nanoparticle of any one of embodiments 1-38, wherein the ionizable lipid is any compound from Tables I-X, the one or more phospholipids comprise a mixture of sphingomyelin and DSPC, the structural lipid is cholesterol, and the PEG lipid is DMG-PEG2k.
57. The lipid nanoparticle of any one of embodiments 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % of a combination of sphingomyelin and DSPC; and (d) about 25 mol % to about 45 mol % of the one or more ionizable lipids.
58. The lipid nanoparticle of any one of embodiments 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % of a combination of sphingomyelin and DSPC; and (d) about 25 mol % to about 45 mol % of an ionizable lipid selected from those in Tables I-X, or any combinations thereof.
59. The lipid nanoparticle of any one of embodiments 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % of a combination of sphingomyelin and DSPC; and (d) about 28 mol % to about 40 mol % of the one or more ionizable lipids.
60. The lipid nanoparticle of any one of embodiments 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % of a combination of sphingomyelin and DSPC; and (d) about 28 mol % to about 40 mol % of an ionizable lipid selected from those in Tables I-X, or any combinations thereof.
61. The lipid nanoparticle of any one of embodiments 1-38, wherein the lipid nanoparticle comprises (a) about 2 mol % of DMG-PEG2k; (b) about 25 mol % cholesterol; (c) about 40 mol % of a combination of sphingomyelin and DSPC; and (d) about 33 mol % of an ionizable lipid selected from those in Tables I-X, or any combinations thereof.
62. The lipid nanoparticle of any one of embodiments 1-38, wherein the lipid nanoparticle comprises (a) about 2 mol % of DMG-PEG2k; (b) about 25 mol % cholesterol; (c) about 40 mol % of a combination of sphingomyelin and DSPC; and (d) about 33 mol % of the one or more ionizable lipids.
63. The lipid nanoparticle of any one of embodiments 1-38, wherein the lipid nanoparticle comprises about 20 mol % 1,2-distearoyl-sn-glycero-3-phosphocholine (DSPC) and about 20 mol % sphingomyelin.
64. The lipid nanoparticle of any one of embodiments 1-63, wherein the lipid nanoparticle further comprises at least one additional lipid component selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-diolcoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Na-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-α-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g. 1,2-dioleoyl-sn-glycero-3-phosphoethanolamine-N-[4-(p-maleimidophenyl) but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol (PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamineB-phosphatidylethanolamine lipid (Rh-PE), purifiedsoy-derivedmixtureofphospholipids (SIOO), phosphatidylcholine (SM), 18-1-trans-PE,1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.
65. The lipid nanoparticle of any one of embodiments 1-66, wherein the lipid nanoparticle further comprising a targeting moiety.
66. The lipid nanoparticle of embodiment 65, wherein the targeting moiety has affinity for an HSC or surface protein thereof.
67. The lipid nanoparticle of embodiment 66, wherein the HSC surface protein is selected from the group consisting of: CD2; 2B4/CD244/SLAMF4; ABCG2; Aldehyde Dehydrogenase 1-A1/ALDH1A1; BMI-1; C1qR1/CD93; CD34; CD38; CD44; CD45; CD48/SLAMF2; CD90/Thy1; CD117/c-kit; CD133; CDCP1; CXCR4; Endoglin/CD105; EPCR; Erythropoietin R; ESAM; EVI-1; Flt-3/Flk-2; GATA-2; GFI-1; Hematopoietic Lineage Marker; Hematopoietic Stem Cells; Integrin alpha 6/CD49f; Mcl-1; MYB; PLZF; Podocalyxin; Prominin 2; PTEN; PU.1/Spi-1; Sca-1/Ly6; SLAM/CD150; Spi-B; STAT5a/b; STAT5a; STAT5b; VCAM-1/CD106; and VEGFR2/KDR/Flk-1.
68. The lipid nanoparticle of embodiment 66, wherein the HSC surface protein is selected from the group consisting of: CD2; CD90; and CD117.
69. The lipid nanoparticle of embodiment 65, wherein the targeting moiety comprises an antibody or antigen-binding fragment thereof selected from IgG2k clone A3C6E2 antibody and IgG2k clone 104D2 antibody.
70. A pharmaceutical composition comprising a lipid nanoparticle of any one of embodiments 1-69 and one or more pharmaceutically acceptable excipients.
71. A cell comprising a lipid nanoparticle or pharmaceutical composition of any one of embodiments 1-70.
72. A cell comprising an edit in a hemoglobin gene or regulatory region thereof installed by a gene editing system of any lipid nanoparticle or pharmaceutical composition of any one of embodiments 1-70.
73. The pharmaceutical composition of embodiment 70 for use as a medicament in treatment of a hemoglobinopathy.
74. The pharmaceutical composition of embodiment 73, wherein the hemoglobinopathy is sickle cell disease (SCD) of transfusion-dependent β-thalassemia (TDT).
75. The pharmaceutical composition of embodiment 73, wherein the hemoglobinopathy is treated by increasing the production of fetal hemoglobin.
76. The pharmaceutical composition of embodiment 75, wherein the production of fetal hemoglobin is increased by disrupting the BCL11A binding site in the HBG1/2 promoters.
77. The pharmaceutical composition of embodiment 75, wherein the production of fetal hemoglobin is increased by disrupting the enhancer sequence of the gene encoding BCL11A.
78. A method of treating a hemoglobinopathy in a subject in need thereof, the method comprising administering to the subject a therapeutically effective amount of a pharmaceutical composition of any one of embodiments 73-77.
79. A method of treating a hemoglobinopathy in a subject in need thereof, the method comprising administering to the subject a therapeutically effective amount of a pharmaceutical composition comprising a lipid nanoparticle comprising:
a) a cargo comprising one or more nucleic acid molecules encoding a type V gene editing system which is capable of installing an edit in a hemoglobin or hemoglobin-associated gene or regulatory region which results in an increased production of fetal hemoglobin;
b) one or more ionizable lipids;
c) one or more phospholipids, in an amount of about 20 mol % to about 60 mol % of the total lipid content of the lipid nanoparticle;
d) one or more structural lipids; and
e) one or more PEG lipids.
80. The method of embodiment any one of embodiments 78 and 79, wherein the therapeutically effective amount of the pharmaceutical composition is a dosage regimen capable of affecting the edit to the hemoglobin or hemoglobin-associated gene or regulatory region in at least 25% of long-term hematopoietic stem cells in a bone marrow sample collected from the subject 28 days after administration.
81. The method of embodiment 79, wherein the one or more nucleic acid molecules are DNA and/or RNA molecules.
82. The method of embodiment 79, wherein the one or more nucleic acid molecules are RNA molecules.
83. The method of embodiment 79, wherein the gene editing system comprises (i) a zinc finger protein, (ii) a TALEN protein, or (iii) a nucleic acid programmable nuclease and a guide RNA.
84. The method of embodiment 79, wherein the gene editing system is a CRISPR-type II gene editing system, a CRISPR-type V gene editing system, or a retron gene editing system.
85. The method of embodiment 84, wherein the retron gene editing system comprises a CRISPR-type II or type V nuclease or functional variant or ortholog thereof, a retron non-coding RNA (ncRNA), and a guide RNA.
86. The method of embodiment 83 or 85, wherein the guide RNA comprises a spacer sequence that is complementary to a target sequence.
87. The method of embodiment 86, wherein the target sequence is a hemoglobin gene or regulatory region thereof.
88. The method of embodiment 87, wherein the hemoglobin gene or regulatory region thereof is the gene encoding hemoglobin beta subunit.
89. The method of embodiment 87, wherein the hemoglobin gene or regulatory region thereof is the BCL11A binding site in the HBG1/2 promoter.
90. The method of embodiment 87, wherein the hemoglobin gene or regulatory region thereof is the enhancer sequence of the gene encoding BCL11A.
91. The method of embodiment 87, wherein the hemoglobin gene or regulatory region thereof is the binding site of the GATA-1 activator in the HBG1/2 promoter.
92. The method of embodiments 83 or 85, wherein the guide RNA comprises a sequence selected from:
   (a) SEQ ID NO: 51;
   (b) SEQ ID NOs: 207-218;
   (c) SEQ ID NOs: 220-537; and
   (d) SEQ ID NOs: 680-720;
      or any portion thereof or a sequence having at least 80%, or 85% or 90%, or 95%, or 99%, or up to 100% sequence identity with any of the aforementioned sequences.
93. The method of embodiments 83 or 85, wherein the guide RNA comprises miRNA sequences selected from:
   (d) SEQ ID NO: 721-735;
   (e) SEQ ID NOs: 721; and
   (f) SEQ ID NOs: 723;
      or any portion thereof or a sequence having at least 80%, or 85% or 90%, or 95%, or 99%, or up to 100% sequence identity with any of the aforementioned sequences.
94. The method of embodiment 84, wherein the CRISPR-type V nuclease comprises SEQ ID NOs: 52, 53, 57-80, 84-93, 96-126, 754-792 or any amino acid sequence having at least 80%, or 85% or 90%, or 95%, or 99%, or up to 100% sequence identity with any of the aforementioned sequences.
95. A lipid nanoparticle comprising:
a) a cargo comprising one or more nucleic acid molecules encoding and/or constituting a gene editing system capable of installing an edit in a hemoglobin gene or regulatory region thereof which results in an increased production of fetal hemoglobin;
b) one or more ionizable lipids in an amount of about 25 mol % to about 45 mol %;
c) one or more phospholipids in an amount of about 30 mol % to about 60 mol %;
d) one or more structural lipids in an amount of about 15 mol % to about 35 mol %; and
e) one or more PEG lipids in an amount of about 1 mol % to about 3 mol %;
   wherein the amount of each lipid component is as a proportion of the total lipid content of the lipid nanoparticle.
96. The lipid nanoparticle of embodiment 95, wherein the lipid nanoparticle does not comprise a targeting moiety.
97. The lipid nanoparticle of any one of embodiments 95-96, wherein the one or more nucleic acid molecules are DNA and/or RNA molecules.
98. The lipid nanoparticle of any one of embodiments 95-97, wherein the one or more nucleic acid molecules are RNA molecules.
99. The lipid nanoparticle of any one of embodiments 95-98, wherein the gene editing system is a CRISPR-type II gene editing system, a CRISPR-type V gene editing system, or a retron gene editing system.
100. The lipid nanoparticle of embodiment 99, wherein the CRISPR-type II gene editing system comprises a CRISPR-type II nuclease or functional variant or ortholog thereof and a guide RNA.
101. The lipid nanoparticle of embodiment 99, where the CRISPR-type V gene editing system comprises a CRISPR-type V nuclease or functional variant or ortholog thereof and a guide RNA.
102. The lipid nanoparticle of any one of embodiments 100 and 101, wherein the guide RNA comprises a spacer sequence that is complementary to a target sequence.
103. The lipid nanoparticle of embodiment 102, wherein the target sequence is a hemoglobin gene or regulatory region thereof.
104. The lipid nanoparticle of any one of embodiments 95-103, wherein the hemoglobin gene or regulatory region thereof is the gene encoding hemoglobin beta subunit.
105. The lipid nanoparticle of any one of embodiments 95-103, wherein the hemoglobin gene or regulatory region thereof is the BCL11A binding site in the HBG1/2 promoter.
106. The lipid nanoparticle of any one of embodiments 95-103, wherein the hemoglobin gene or regulatory region thereof is the enhancer sequence of the gene encoding BCL11A.
107. The lipid nanoparticle of any one of embodiments 95-103, wherein the hemoglobin gene or regulatory region thereof is the binding site of the GATA-1 activator in the HBG1/2 promoter.
108. The lipid nanoparticle any one of embodiments 101-107, wherein the type V guide RNA comprises a sequence selected from SEQ ID NOs: 51, 207-218, or any portion thereof or a sequence having at least 80%, or 85% or 90%, or 95%, or 99%, or up to 100% sequence identity with any of the aforementioned sequences.
109. The lipid nanoparticle of any one of embodiments 101-107, wherein the guide RNA comprises SEQ ID NOs: 220-537 and 680-720, or any portion thereof or a sequence having at least 80%, or 85% or 90%, or 95%, or 99%, or up to 100% sequence identity with any of the aforementioned sequences.
110. The lipid nanoparticle of any one of embodiments 101-107, wherein the CRISPR-type V nuclease comprises SEQ ID NOs: 52, 53, 57-80, 84-93, 96-126, 754-792 or any amino acid sequence having at least 80%, or 85% or 90%, or 95%, or 99%, or up to 100% sequence identity with any of the aforementioned sequences.
111. The lipid nanoparticle of any one of embodiments 95-110, wherein the gene editing system comprises one or more additional accessory proteins.
112. The lipid nanoparticle of embodiment 111, wherein the one or more accessory proteins comprises a nuclease, reverse transcriptase, recombinase, transposase, integrase, DNA binding protein, transcription factor, a CRISPR Cas protein, or an anti-CRISPR inhibitor protein.
113. The lipid nanoparticle of embodiment 112, wherein the CRISPR Cas protein is Cas1, Cas2, Cas3, Cas4, Cas5, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas13, Cas13a (C2c2), Cas13b, Cas13c, Cas13d, Cas13x.1, Cse1, Cse2, Csy1, Csy2, Csy3, Csm2, Cmr5, Csx10, Csf1, Csn2, C2c4, C2c8, or C2c9.
114. The lipid nanoparticle of any one of embodiments 111-113, wherein the one or more accessory proteins may be provided in trans or may be coupled to one or more components of the gene editing system.
115. The lipid nanoparticle of any one of embodiments 111-113, wherein the one or more accessory proteins is coupled to one or more components of the gene editing system by a linker.
116. The lipid nanoparticle of embodiment 115, wherein the linker has an amino acid sequence of SEQ ID NOs: 33-37 and 81-83.
117. The lipid nanoparticle of any one of embodiments 95-117, wherein the N:P ratio is from about 5:1 to about 8:1.
118. A pharmaceutical composition comprising a lipid nanoparticle of any one of embodiments 111-113, and one or more pharmaceutical excipients.
119. A cell comprising a lipid nanoparticle of embodiments 95-117 or a pharmaceutical composition of embodiment 118.
120. A cell comprising an edit in a hemoglobin gene or regulatory region thereof installed by a gene editing system of any lipid nanoparticle of embodiments 95-117 or a pharmaceutical composition of embodiment 118.
121. The pharmaceutical composition of embodiment 118 for use as a medicament in treatment of a hemoglobinopathy.
122. The pharmaceutical composition of embodiment 121, wherein the hemoglobinopathy is sickle cell disease (SCD) of transfusion-dependent β-thalassemia (TDT).
123. The pharmaceutical composition of embodiment 121, wherein the hemoglobinopathy is treated by increasing the production of fetal hemoglobin.
124. The pharmaceutical composition of embodiment 123, wherein the production of fetal hemoglobin is increased by disrupting the BCL11A binding site in the HBG1/2 promoters.
125. The pharmaceutical composition of embodiment 123, wherein the production of fetal hemoglobin is increased by disrupting the enhancer sequence of the gene encoding BCL11A.
126. A method of treating a hemoglobinopathy in a subject in need thereof, the method comprising administering to a subject a therapeutically effective amount of a pharmaceutical composition of any one of embodiments 121-125.
127. A lipid nanoparticle for delivery of a nucleic acid cargo to a hematopoietic stem cell in vivo to treat a hemoglobinopathy, the lipid nanoparticle comprising:
a) a cargo comprising one or more nucleic acid molecules encoding a type V gene editing system which is capable of installing an edit in a hemoglobin or hemoglobin-associated gene or regulatory region which results in an increased production of fetal hemoglobin;
b) one or more ionizable lipids selected from any of the compounds of Tables (I)-(X) in an amount of about 33 mol %;
c) one or more phospholipids in an amount of about 40 mol %;
d) one or more structural lipids in an amount of about 25 mol %; and
e) one or more PEG lipids in an amount of about 2 mol %;
   wherein the amount of each lipid component is as a proportion of the total lipid content of the lipid nanoparticle.
128. The lipid nanoparticle of embodiment 127, wherein the lipid nanoparticle does not comprise a targeting moiety.
129. The lipid nanoparticle of embodiment any one of embodiments 127-128, wherein the lipid nanoparticle passively delivers to hematopoietic stem cells in vivo.
130. The lipid nanoparticle of any one of embodiments 127-129, wherein the one or more nucleic acid molecules are DNA and/or RNA molecules.
131. The lipid nanoparticle of any one of embodiments 127-130, wherein the one or more nucleic acid molecules are RNA molecules.
132. The lipid nanoparticle of any one of embodiments 127-131, wherein the CRISPR-type V gene editing system comprises a CRISPR-type V nuclease or functional variant or ortholog thereof and a guide RNA.
133. The lipid nanoparticle of embodiment 132, wherein the guide RNA comprises a spacer sequence that is complementary to a target sequence.
134. The lipid nanoparticle of any one of embodiments 127-133, wherein the target sequence is a hemoglobin gene or regulatory region thereof.
135. The lipid nanoparticle of any one of embodiments 127-134, wherein the hemoglobin gene or regulatory region thereof is the BCL11A binding site in the HBG1/2 promoter.
136. The lipid nanoparticle of any one of embodiments 127-134, wherein the hemoglobin gene or regulatory region thereof is the enhancer sequence of the gene encoding BCL11A.
137. The lipid nanoparticle of any one of embodiments 132-136, wherein the type V guide RNA comprises a sequence selected from:
   (a) SEQ ID NO: 51;
   (b) SEQ ID NOs: 207-218;
   (c) SEQ ID NOs: 220-537; and
   (d) SEQ ID NOs: 680-720;
      or any portion thereof or a sequence having at least 80%, or 85% or 90%, or 95%, or 99%, or up to 100% sequence identity with any of the aforementioned sequences.
138. The lipid nanoparticle of any one of embodiments 132-136, wherein the guide RNA comprises miRNA sequences selected from:
   (a) SEQ ID NO: 721-735;
   (b) SEQ ID NOs: 721; and
   (c) SEQ ID NOs: 723;
      or any portion thereof a sequence having at least 80%, or 85% or 90%, or 95%, or 99%, or up to 100% sequence identity with any of the aforementioned sequences
139. The lipid nanoparticle of any one of embodiments 132-136, wherein the CRISPR-type V nuclease comprises SEQ ID NOs: 52, 53, 57-80, 84-93, 96-126, 754-792 or any amino acid sequence having at least 80%, or 85% or 90%, or 95%, or 99%, or up to 100% sequence identity with any of the aforementioned sequences.
140. The lipid nanoparticle of any one of embodiments 127-139, wherein the N:P ratio is from about 5:1 to about 8:1.
141. The lipid nanoparticle of any one of embodiments 127-140, further comprising a targeting moiety.
142. The lipid nanoparticle of embodiment 141, wherein the targeting moiety has affinity for an HSC or surface protein thereof.
143. The lipid nanoparticle of embodiment 142, wherein the HSC surface protein is selected from the group consisting of: CD2; 2B4/CD244/SLAMF4; ABCG2; Aldehyde Dehydrogenase 1-A1/ALDH1A1; BMI-1; C1qR1/CD93; CD34; CD38; CD44; CD45; CD48/SLAMF2; CD90/Thy1; CD117/c-kit; CD133; CDCP1; CXCR4; Endoglin/CD105; EPCR; Erythropoietin R; ESAM; EVI-1; Flt-3/Flk-2; GATA-2; GFI-1; Hematopoietic Lineage Marker; Hematopoietic Stem Cells; Integrin alpha 6/CD49f; Mcl-1; MYB; PLZF; Podocalyxin; Prominin 2; PTEN; PU.1/Spi-1; Sca-1/Ly6; SLAM/CD150; Spi-B; STAT5a/b; STAT5a; STAT5b; VCAM-1/CD106; and VEGFR2/KDR/Flk-1.
144. The lipid nanoparticle of embodiment 142, wherein the HSC surface protein is selected from the group consisting of: CD2; CD90; and CD117.
145. A pharmaceutical composition comprising a lipid nanoparticle of any one of embodiments 127-144 and one or more pharmaceutical excipients.
146. A cell comprising a lipid nanoparticle of embodiments 127-145 or a pharmaceutical composition of embodiment 146.
147. A cell comprising an edit in a hemoglobin gene or regulatory region thereof installed by a gene editing system of any lipid nanoparticle of embodiments 127-145 or a pharmaceutical composition of embodiment 146.
148. The pharmaceutical composition of embodiment 146 for use as a medicament in treatment of a hemoglobinopathy.
149. The pharmaceutical composition of embodiment 148, wherein the hemoglobinopathy is sickle cell disease (SCD) of transfusion-dependent β-thalassemia (TDT).
150. The pharmaceutical composition of any one of embodiments 148-149, wherein the hemoglobinopathy is treated by increasing the production of fetal hemoglobin.
151. The pharmaceutical composition of embodiment 150, wherein the production of fetal hemoglobin is increased by disrupting the BCL11A binding site in the HBG1/2 promoters.
152. The pharmaceutical composition of embodiment 150, wherein the production of fetal hemoglobin is increased by disrupting the enhancer sequence of the gene encoding BCL11A.
153. A method of treating a hemoglobinopathy, the method comprising administering to a subject in need thereof a therapeutically effective amount of a pharmaceutical composition of any one of embodiments 148-152.

### EXAMPLES

The present disclosure will be more fully understood by reference to the following examples, which provide illustrative non-limiting aspects of the present disclosure.

### Example 1: Synthesis of Lipids

Ionizable lipids and PEG lipids of Series “A”, “CY”, “C”, “CX”, “CZ”, “AT”, “AC”, “CC”, “CO”, “S” and “PL” were prepared as reported in PCT Publications WO2023044343A1, WO2023044333A1, WO2023122752A1, WO2024044728A1 and WO2023196931A1 and PCT Application PCT/US2024/019990 using appropriate starting materials and procedures as would be apparent to a person of ordinary skill in the art. Additional lipids of the present disclosure were made by methods and procedures as would be apparent to a person of ordinary skill in the art. Exemplary additional synthetic procedures and schema are included below.

### Synthesis of Selected Intermediates

### Synthesis of 4,4-bis(3,7-dimethyloctyl)oxy) butane nitrile (L4L-2) [Procedure A]

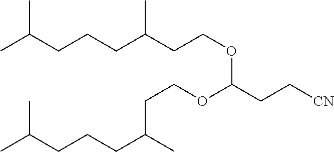 [SMILES: CC(C)CCCC(C)CCOC(CCC#N)OCCC(C)CCCC(C)C]

To a 100 mL round bottom flask, 4,4-dimethoxybutanenitrile (3.0 g, 23.2 mmol), 3,7-dimethyloctan-1-ol (11.0 g, 69.7 mmol) and pyridinium p-toluenesulfonate (0.29 g 1.2 mmol) were added. The resulting mixture was stirred at 120° C. for 4 h and cooled to room temperature. EtOAc (50 mL) and H₂O (20 mL) were added in, and the resulting phases were separated. The aqueous phase was extracted with EtOAc (50 mL). Combined organic extracts were washed with H₂O (20 mL) and dried over anhydrous MgSO₄. Filtration and concentration provided crude material which was purified by flash column chromatography (SiO₂: 0 to 10% ethyl acetate in hexanes gradient) to yield L4L-2 as colorless oil (6.6 g, 74%); ¹HNMR (CDCl₃) δ 4.50-4.53 (t, 1H), 3.58-3.60 (m, 2H), 3.41-3.49 (m, 2H), 2.39-2.44 (t, 2H), 1.92-1.94 (q, 2H), 1.50-1.55 (m, 6H), 1.38-1.42 (m, 2H), 1.11-1.14 (m, 14H) 0.88-0.84 (t, 18H); CIMS m/z [M+H]⁺ 381.

### Synthesis of 4,4-bis((3,7-dimethyloctyl)oxy) butanoic acid (L4L-3) [Procedure B]

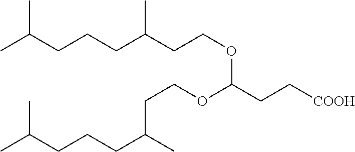 [SMILES: CC(C)CCCC(C)CCOC(CCC(=O)O)OCCC(C)CCCC(C)C]

To a 100 mL round bottom flask containing a solution of L4L-2 (8.2 g, 21 mmol) in ethanol (50 mL) was added a solution of KOH (3.6 g, 64 mmol) in water (50 mL). After completion of addition, the mixture was stirred at 120° C. for 20 h. The volatiles were removed, and the reaction pH was adjusted to 5. EtOAc (150 mL) and H₂O (60 mL) were added, and the resulting phases were separated. The aqueous phase was extracted with EtOAc (50 mL). Combined organic extracts were washed with H₂O (60 mL×2) and dried over anhydrous MgSO₄. Filtration and concentration provided L4L-3 (6.4 g, 74%) which was used for the next step without further purification. ¹HNMR (CDCl₃) δ 4.54 (t, 1H), 3.60-3.65 (m, 2H), 3.45-3.49 (m, 2H), 2.39-2.44 (t, 2H), 1.92-1.94 (m, 2H), 1.50-1.95 (m, 6H), 1.26-1.55 (m, 8H), 1.11-1.14 (m, 6H). 0.84-0.88 (d, 18H); CIMS m/z [M−H]⁻ 399.

### Synthesis of 4,4-bis(octyloxy) butanoic acid (L4L-4)

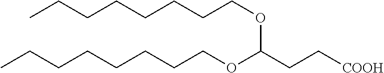 [SMILES: CCCCCCCCOC(CCC(=O)O)OCCCCCCCC]

Prepared following Procedures A & B described in Compound L4L-3 synthesis, replacing 3,7-dimethyloctan-1-ol with octan-1-ol. Compound L4L-4 was isolated as light-yellow oil in a yield of 11.8 g (98%). ¹HNMR (CDCl₃) δ: 4.53-4.56 (t, 1H), 3.57-3.60 (m, 2H), 3.40-3.43 (m, 2H), 2.39-2.41 (t, 2H), 1.90-1.95 (m, 2H), 1.54-1.56 (M, 4H), 1.26 (bs, 28H), 0.85-0.87 (t, 6H); CIMS m/z [M−H]⁻ 371.

### Synthesis of 2,2′-(1-(4-(benzyloxy)butyl) piperidine-4,4-diyl)bis(ethan-1-ol) (L27-3)

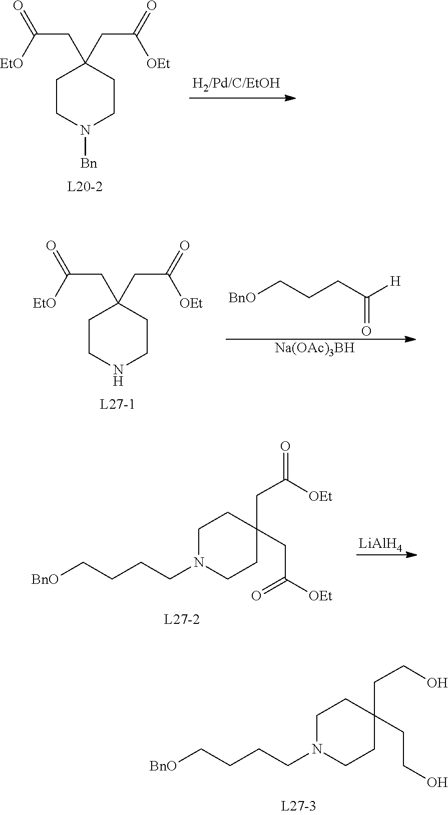

### Synthesis of diethyl 2,2′-(piperidine-4,4-diyl)diacetate (L27-1)

A solution of L20-2 (5.4 g, 15.4 mmol) in ethanol (107 ml) at room temperature was treated with 10% Pd/C (1.1 g) under nitrogen atmosphere. The reaction mixture was evacuated and flushed with II2 gas (3×) and then stirred vigorously under an atmosphere of II2 (1 atm, II2-balloon) at room temperature. After 24 h, the reaction mixture was filtered through Celite and the filtrate was concentrated in vacuo to give the crude product, L27-1 (4 g) which was used for the next step without further purification. APCI MS m/z [M+H]⁺ 257.16.

### Synthesis of diethyl 2,2′-(1-(4-(benzyloxy)butyl) piperidine-4,4-diyl)diacetate (L27-2)

To a mixture of L27-1 (4 g, 15.5 mmol) and 4-(benzyloxy) butanal (5.5 g, 31.1 mmol) in 1,2-dichloroethane (180 mL) was added Na(OAc)₃BH (9.9 g, 46.6 mmol) and acetic acid (1 mL). The reaction mixture was subjected to vacuum/N₂ cycle (3×) and stirred at room temperature for 18 h. The reaction was quenched by slow addition of saturated NaHCO₃ (100 mL) at 0° C. The aqueous phase was extracted using ethyl acetate (100 mL, 3×) and the combined organic phases were dried over anhydrous Na₂SO₄. Filtration followed by concentration provided crude material, which was dissolved in DCM. Silica gel (40 g) and triethyl amine (40 mL) were added to the crude material and shaken for 10-15 min and the solvent was removed under vacuum. The residue was loaded on to an empty flash cartridge, which was then attached to flash purification system loaded with 80 g flash silica column and was purified by flash chromatography (SiO₂: 0 to 10% ethyl acetate in hexane (10% triethylamine)) to yield ethyl L27-2 as slightly yellow oil (3.7 g, 57%). ¹H-NMR (300 MHz, CDCl₃) δ 7.31-7.30 (m, 5H), 4.46 (s, 2H), 4.09-4.04 (m, 4H), 3.47-3.43 (m, 2H), 2.52-2.31 (m, 10H), 1.68-1.57 (m, 8H), 1.22 (t, 6H); APCI MS m/z [M+H]⁺ 420.3.

### Synthesis of 2,2′-(1-(4-(benzyloxy)butyl) piperidine-4,4-diyl)bis(ethan-1-ol) (L27-3)

A solution of L27-2 (0.75 g, 1.78 mmol) in THF (14 mL) was cooled in an ice bath (0° C.) and to this was added 2M LiAlH₄ in THF (3.56 mL, 7.14 mmol), dropwise. The ice bath was removed, and the reaction mixture was stirred for 18 h at room temperature. The mixture was diluted with Et₂O (50 mL), cooled in an ice bath, and carefully quenched with water (10 mL), 20% NaOH (10 mL) and water (30 mL). After stirring for 30 min, the aqueous phase was extracted with 20 mL DCM (3×), then the combined organic phase was dried (Na₂SO₄), filtered and concentrated to give L27-3 (0.54 g, 91% yield) as a white solid. APCI MS m/z [M+H]⁺ 336.3.

### Synthesis of S-2

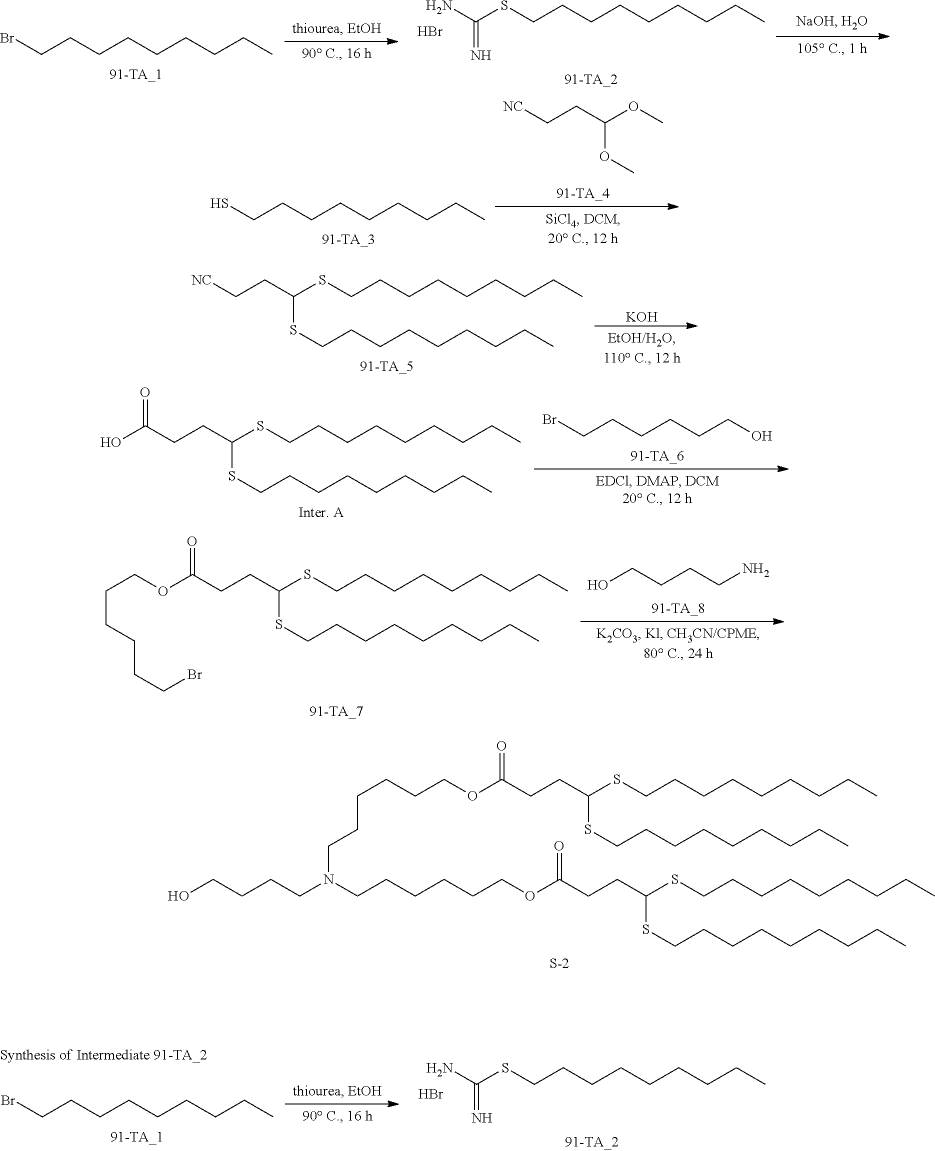

To a solution of 91-TA_1 (25 g, 120.69 mmol) in EtOH (100 mL) was added a solution of isothiourea (11 g, 144.82 mmol) in EtOH (100 mL) at 25° C. The mixture was stirred at 90° C. for 12 h. TLC (eluted with petroleum ether:ethyl acetate=50:1, PMA, R_{f}=0.3) indicated 20% of 91-TA_1 was consumed, and one new spot formed. The mixture was filtered and the filter cake was concentrated under reduced pressure to give 91-TA_2 (55 g, crude) as white solid. ¹H NMR: (400 MHz CDCl₃-d) δ=3.16 (t, J=7.2 Hz, 2H), 1.72 (m, 2H), 1.52-1.24 (m, 12H), 0.97-0.84 (m, 3H).

### Synthesis of Intermediate 91-TA_3

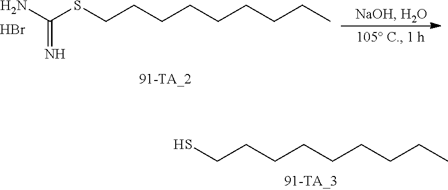

To a solution of NaOH (23.92 g, 598.08 mmol) in H₂O (2400 mL) was added 91-TA_2 (51.5 g, 254.5 mmol) at 20° C. The mixture was stirred at 105° C. for 1 h. TLC (eluted with petroleum ether:ethyl acetate=10:1, PMA, R_{f}=0.97) showed 2-nonylisothiourea was consumed and one new spot formed. The residue was extracted with methyl tert-butyl ether (2000 mL×3). The combined organic layers were dried over Na₂SO₄, filtered and concentrated under reduced pressure to give 91-TA_3 (40 g, crude) as a yellow oil. ¹H NMR: (400 MHz CDCl₃-d) δ=4.98 (s, 2H), 2.59 (t, J=7.2 Hz, 2H), 1.69 (m, 2H), 1.53-1.39 (m, 11H), 1.05-0.97 (m, 3H)

### Synthesis of Intermediate 91-TA_5

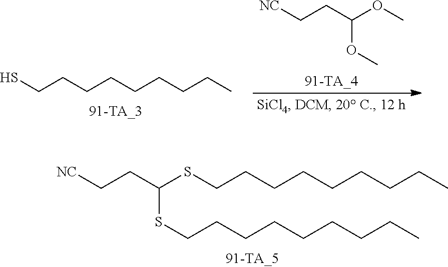

To a solution of 91-TA_4 (6.5 g, 50.33 mmol) and 91-TA_3 (16.14 g, 100.65 mmol) in DCM (130 mL) was added SiCl₄ (1.71 g, 10.07 mmol, 1.16 mL) at 20° C. The mixture was stirred at 20° C. for 12 h. TLC (eluted with petroleum ether:ethyl acetate=20:1, PMA, R_{f}=0.58) showed 91-TA_4 was consumed, 91-TA_3 was remained and one new spot formed. The residue was diluted with H2O 50 mL and extracted with dichloromethane (50 mL×3). The combined organic layers were dried over Na₂SO₄, filtered and concentrated under reduced pressure to give a residue. The resulting mixture was concentrated to give a dry flowing solid, and then it was loaded to an 80 g Agela flash silica gel column (Biotage) eluted with 0% to 5% ethyl acetate in petroleum ether gradient to give 91-TA_5 (14 g, 36.06% yield) as a yellow oil. ¹H NMR: (400 MHz CDCl₃-d) δ=3.84 (t, J=7.2 Hz, 1H), 2.73-2.50 (m, 6H), 2.17-2.05 (m, 2H), 1.63-1.54 (m, 4H), 1.45-1.25 (m, 24H), 0.88 (t, J=6.4 Hz, 6H)

### Synthesis of Intermediate Inter. A

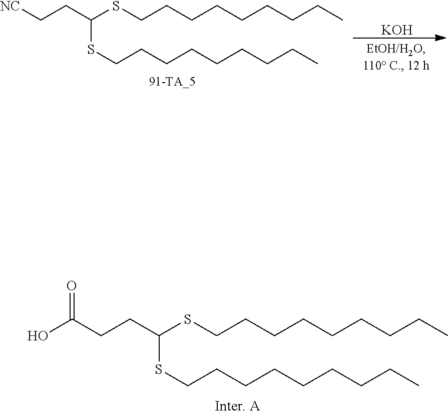

To a solution of 91-TA_5 (5 g, 12.96 mmol) in EtOH (50 mL) and H₂O (50 mL) was added KOH (5.82 g, 103.70 mmol) at 0° C. The mixture was stirred at 110° C. for 12 h. TLC (eluted with petroleum ether:ethyl acetate=10:1, PMA, R_{f}=0.29) showed 91-TA_5 was consumed and one new spot formed. The reaction mixture was concentrated under reduced pressure to remove solvent. The mixture was adjusted to pH=5 by HCl (6 M, 30 mL). The residue was extracted with ethyl acetate (50 mL×3). The combined organic layers were dried over Na₂SO₄, filtered and concentrated under reduced pressure to give Inter. A (6.2 g, crude) as a yellow oil. ¹H NMR: (400 MHz CDCl₃-d) δ=3.79 (t, J=6.8 Hz, 1H), 2.72-2.50 (m, 6H), 2.11-2.01 (m, 2H), 1.58 (m, 4H), 1.47-1.26 (m, 24H), 0.89 (t, J=6.4 Hz, 6H)

### Synthesis of Intermediate 91-TA_7

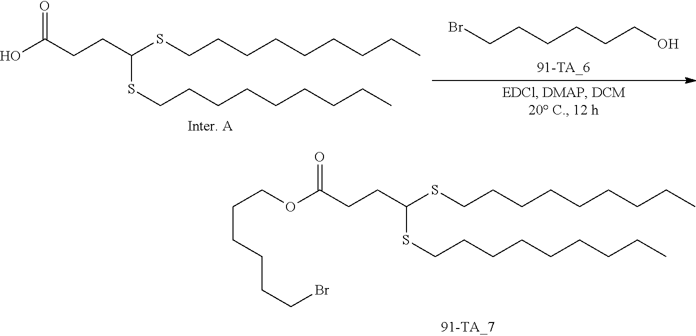 [SMILES: CCCCCCCCCSC(CCC(=O)O)SCCCCCCCCC ; OCCCCCCBr ; CCCCCCCCCSC(CCC(=O)OCCCCCCBr)SCCCCCCCCC]

To a solution of Inter. A (5 g, 12.35 mmol) in DCM (50 mL) was added EDCI (2.84 g, 14.83 mmol) and DMAP (754 mg, 6.18 mmol) at 20° C. 91-TA_6 (4.47 g, 24.71 mmol) in DCM (30 mL) was added at 20° C. The mixture was stirred at 20° C. for 12 h. LCMS showed Inter. A was consumed completely and 50% peak with desired ms (RT=4.637) was detected. TLC (eluted with petroleum ether:ethyl acetate=10:1, PMA, R_{f}=0.77) showed Inter. A was

consumed and one new spot formed. The residue was diluted with H₂O 30 mL and extracted with dichloromethane (50 mL×3). The combined organic layers were dried over Na₂SO₄, filtered and concentrated under reduced pressure to give a residue. The resulting mixture was concentrated to give a dry flowing solid, and then it was loaded to a 40 g Agela flash silica gel column (Biotage), eluted with 0% to 15% ethyl acetate in petroleum ether gradient to give 91-TA_7 (3.9 g, 55.60% yield) as a yellow oil. ¹H NMR: (400 MHz CDCl₃-d) δ=4.08 (t, J=6.8 Hz, 2H), 3.81 (t, J=7.2 Hz, 1H), 3.42 (t, J=6.0 Hz, 2H), 2.71-2.51 (m, 6H), 2.10 (q, J=7.2 Hz, 2H), 1.88 (m, 2H), 1.70-1.27 (m, 34H), 0.92-0.85 (t, J=6.4 Hz, 6H)

### General Procedure for the Preparation of S-2

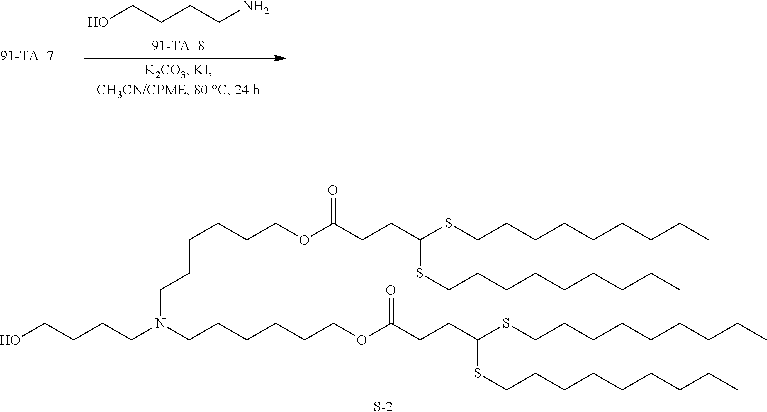

To a solution of 91-TA_7 (3.9 g, 6.87 mmol) in MeCN (20 mL) and CPME (20 mL) was added KI (3.42 g, 20.61 mmol) and K₂CO₃ (3.32 g, 24.04 mmol). And then 4-aminobutan-1-ol (306 mg, 3.43 mmol) was added at 20° C. The mixture was stirred at 80° C. for 16 h. LCMS showed 91-TA_7 was consumed completely and 50% peak with desired ms (RT=4.256) was detected. The resultant mixture was filtered and the filter cake was rinsed with ethyl acetate (30 mL×3). Then the combined filtrates were concentrated under reduced pressure. The resulting mixture was concentrated to give a dry flowing solid, and then it was loaded to Biotage using a 40 g Agela flash silica gel column, eluted with 0% to 100% ethyl acetate in petroleum ether gradient to give S-2 (1.96 g, 26.87% yield) as a yellow oil. LCMS Method: (The column used for chromatography was a XSelect CSH phenyl hexyl 2.1×50 mm, 3.5 μm. Detection methods are diode array (DAD) and evaporative light scattering (ELSD). MS mode was positive electrospray ionization. MS range was 100-1500. Mobile Phase A: 0.04% TFA in water and mobile phase B was 0.02% TFA in HPLC grade acetonitrile. The gradient 50%-100% B in 4.0 minutes and holding at 100% for 1.5 minutes. The flow rate was 1 mL/min. r.t.=4.232 min. ¹H NMR: (400 MHz CDCl₃-d) δ=4.07 (t, J=6.8 Hz, 4H), 3.81 (t, J=7.2 Hz, 2H), 3.57 (br s, 2H), 2.71-2.43 (m, 18H), 2.10 (q, J=7.6 Hz, 4H), 1.73-1.51 (m, 20H), 1.44-1.25 (m, 56H), 0.89 (t, J=6.4 Hz, 12H). The following compounds were prepared using the synthetic procedures described for S-2 except that the starting material was replaced with the starting material as shown in the table.

### Synthesis of S-3 and S-4

| Compound | Starting materials | Characterization |
|---|---|---|
| S-3 | 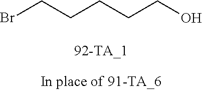 | ¹H NMR: (400 MHz CDCl₃) δ = 4.00 (t, J = 6.8 Hz, 4H), 3.73 (t, J = 7.2 Hz, 2H), 3.49 (br s, 2H), 2.63-2.34 (m, 18H), 2.03 (q, J = 7.2 Hz, 4H), 1.63-1.42 (m, 20H), 1.37-1.17 (m, 52H), 0.81 (t, J = 6.4 Hz, 12H); r.t. = 4.112 min, m/z = 1034.6 (M + H)⁺ |
| S-4 | 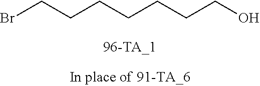 | ¹H NMR: (400 MHz CDCl₃) δ = 4.06 (t, J = 6.8 Hz, 4H), 3.80 (t, J = 7.2 Hz, 2H), 3.56 (br s, 2H), 2.69-2.52 (m, 12H), 2.45 (br t, J = 7.6 Hz, 6H), 2.10 (q, J = 7.2 Hz, 4H), 1.74-1.25 (m, 80H), 0.88 (t, J = 6.4 Hz, 12H); r.t. = 4.357 min, m/z = 1090.7 (M + H)⁺ |

### Synthesis of S-1

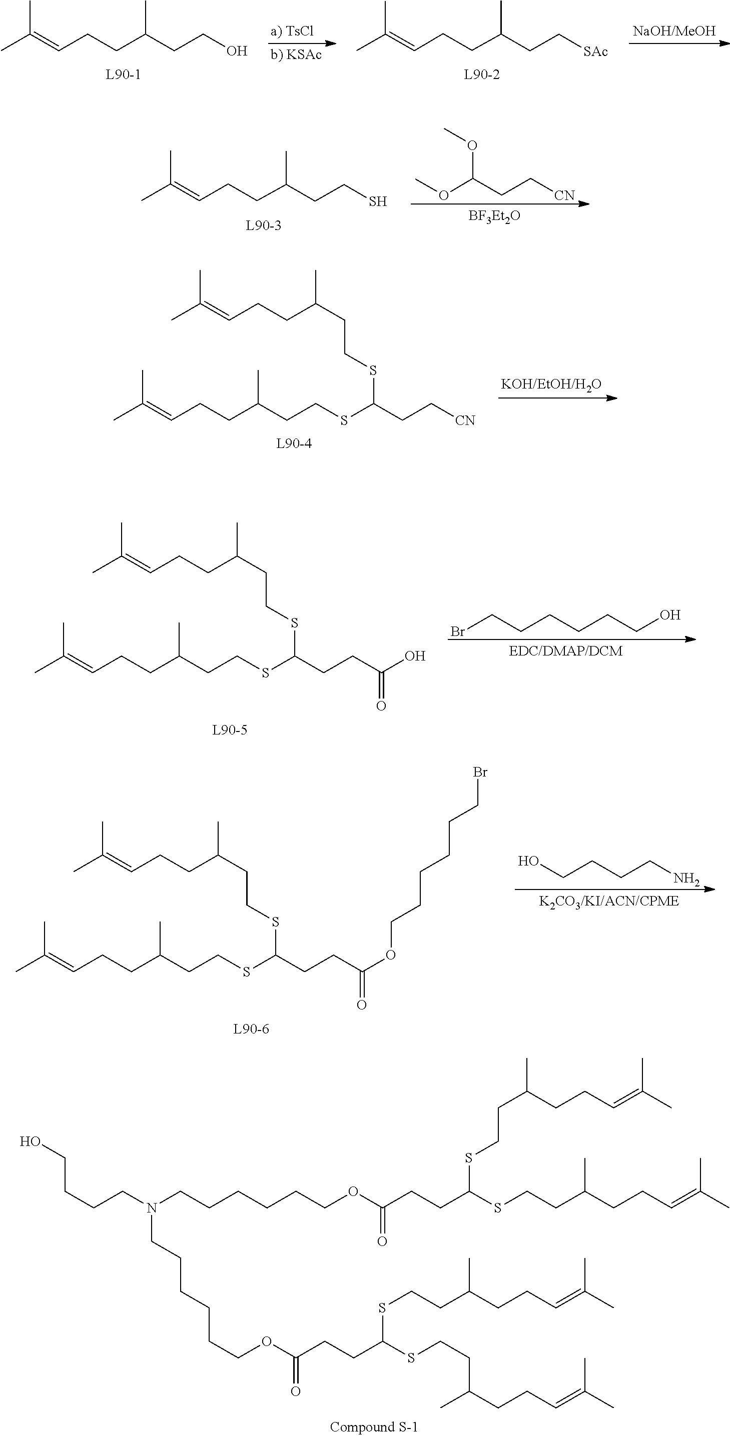

### Synthesis of S-(3,7-dimethyloct-6-en-1-yl) ethanethioate Intermediate L90-2

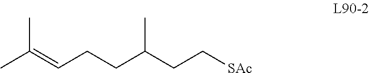

To an ice bath cooled solution of L90-1 (10 g, 64.1 mmol) in anhydrous DCM (100 mL), p-toluenesulfonyl chloride (13.4 g, 70.5 mmol) and DIPEA (24.7 mL, 192.3 mmol) were added, followed by the addition of DMAP (780 mg, 6.4 mmol). The reaction mixture was stirred at room temperature overnight and TLC showed the reaction was incomplete. p-Toluenesulfonyl chloride (12.1 g, 64.1 mmol) and DMAP (780 mg, 6.4 mmol) were added, and reaction mixture was stirred at room temperature for 2 h. Water was added to the reaction mixture and DCM layer separated, aqueous layer was extracted with DCM (100 mL). Combined organic layers were dried over anhydrous Na₂SO₄ and concentrated under reduced pressure to provide crude product which was purified by flash chromatography (SiO₂: 0-10% EtOAc in hexane gradient) to afford the tosylate intermediate 3,7-dimethyloct-6-en-1-yl 4-methylbenzenesulfonate (12.8 g, 64%). ¹H-NMR (300 MHz, CDCl₃) δ: 7.78 (d, J=6.6, 2H), 7.35 (d, J=7.9, 2H), 5.01 (m, 1H), 4.05 (m, 2H), 2.44 (s, 3H), 1.82-1.98 (m, 2H), 1.41-1.68 (m, 9H), 1.07-1.25 (m, 2H), 0.81 (d, J=6.3, 3H). The tosylate intermediate (12.8 g, 41.2 mmol) was dissolved in anhydrous DMF (70 mL) and potassium thioacetate (9.41 g, 82.5 mmol) was added to the reaction mixture. The reaction mixture was heated at 80° C. for 2 h and then diluted with EtOAc (300 mL). The organic layer was washed with water (2×200 mL) and brine (200 mL), dried over Na₂SO₄, concentrated under reduced pressure to give crude product which was purified by flash chromatography (SiO₂: 0-10% EtOAc in hexane gradient) to afford L90-2 (8.1 g, 95%). ¹H-NMR (300 MHz, CDCl₃) δ: 5.07 (m, 1H), 2.82-2.89 (m, 2H), 2.31 (s, 3H), 1.91-1.98 (m, 2H) 1.67 (s, 3H), 1.59 (s, 3H), 1.13-1.54 (m, 5H), 0.89 (d, J=6.6, 3H).

### Synthesis of 3,7-dimethyloct-6-ene-1-thiol Intermediate L90-3

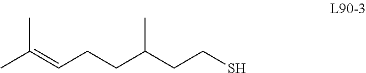

To a solution of L90-2 (8.1 g, 37.85 mmol) in methanol (120 mL) was added 2.0 N NaOH solution (80 mL) and the reaction mixture was stirred at room temperature for 2 h. The reaction mixture was cooled to ice-bath temperature and neutralized by addition of 3.0 N HCl solution. After extraction three times with EtOAc (200 mL and 100 mL×2), the combined organic layers were washed with brine (100 mL), dried over anhydrous Na₂SO₄ and filtered. The filtrate was concentrated under reduced pressure to afford L90-3 as yellowish oil (5.8 g, 89%). The crude product was used for next step without further purification. ¹H-NMR (300 MHz, CDCl₃) δ: 5.08 (m, 1H), 2.49-2.54 (m, 2H), 1.91-1.99 (m, 2H), 1.68 (s, 3H), 1.59 (s, 3H), 1.08-1.49 (m, 5H), 0.88 (d, J=6.3, 3H).

### Synthesis of 4,4-bis((3,7-dimethyloct-6-en-1-yl)thio) butanenitrile Intermediate L90-4

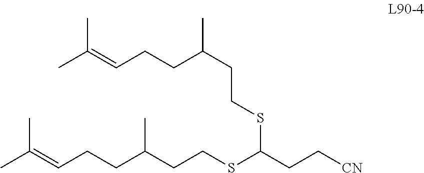 [SMILES: CC(C)=CCCC(C)CCSC(CCC#N)SCCC(C)CCC=C(C)C]

BF₃·OEt₂ (235 μL, 1.92 mmol) was added to a mixture of L90-3 (5.8 g, 33.72 mmol) and cyanopropionaldehyde dimethyl acetal (1.24 g, 9.63 mmol) in anhydrous DCM (60 mL) at 0° C. under N₂ atmosphere. The reaction mixture was stirred at 0° C. for 30 min and then at room temperature overnight. Formation of product was confirmed by LC-MS. The reaction mixture was diluted with DCM (150 mL), and then washed with water (100 mL) and brine (100 mL). The organic layer was dried over anhydrous Na₂SO₄ and concentrated under reduced pressure to get crude product, which was purified by flash chromatography (SiO₂: 0-5% EtOAc in hexane gradient) to afford L90-4 as light-yellow oil (2.8 g, 71%). ¹H-NMR (300 MHz, CDCl₃) δ: 5.08 (m, 2H), 3.83 (t, J=7.26, 1H) 2.58-2.65 (m, 6H), 2.1 (q, J=7.14, 2H), 1.94-1.99 (m, 4H), 1.67 (s, 6H), 1.6 (s, 6H), 1.13-1.58 (m, 10H), 0.89 (m, 6H); CIMS m/z [M+H]⁺ 410.3.

### Synthesis of 4,4-bis((3,7-dimethyloct-6-en-1-yl)thio) butanoic acid Intermediate L90-5

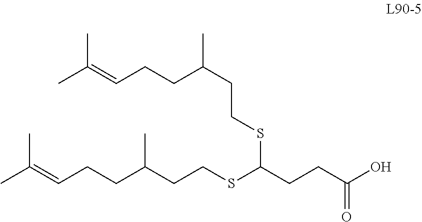 [SMILES: CC(C)=CCCC(C)CCSC(CCC(=O)O)SCCC(C)CCC=C(C)C]

To a mixture of L90-4 (2.7 g, 6.58 mmol) in ethanol (25 mL) was added a solution of KOH (1.1 g, 19.74 mmol) in water (25 mL) in a high pressure tube. The tube was sealed and heated to 110° C. and stirred for 48 h. The flask was cooled to room temperature, transferred to round bottom flask and ethanol was evaporated under reduced pressure. The reaction mixture was cooled in an ice-water bath and acidified to pH=4.5 by slow addition of 1.0 N HCl with addition funnel. The mixture was extracted with EtOAc, the organic layer was washed with water, brine and dried over anhydrous Na₂SO₄, concentrated under reduced pressure to get crude product, which was purified by flash chromatography (SiO₂: 0-20% EtOAc in hexane gradient) to yield L90-5 as colorless oil (2.25 g, 80% yield). ¹H-NMR (300 MHz, CDCl₃) δ: 5.08 (m, 2H), 3.8 (t, J=7.14, 1H) 2.58-2.69 (m, 6H), 2.09 (q, J=7.14, 2H), 1.8-1.99 (m, 4H), 1.67 (s, 6H), 1.59 (s, 6H), 1.13-1.56 (m, 10H), 0.88 (m, 6H); CIMS m/z [M−H]⁺ 427.0.

### Synthesis of 6-bromohexyl 4,4-bis((3,7-dimethyloct-6-en-1-yl)thio)butanoate Intermediate L90-6

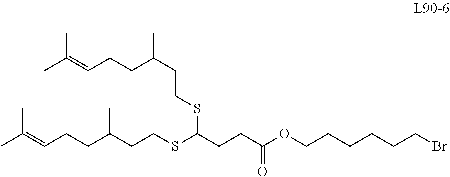 [SMILES: CC(C)=CCCC(C)CCSC(CCC(=O)OCCCCCCBr)SCCC(C)CCC=C(C)C]

To a solution of L90-5 (2.25 g, 5.24 mmol) in DCM (50 mL) were added DMAP (639 mg, 5.24 mmol) and EDC (4.0 g, 20.9 mmol) under nitrogen. The reaction mixture was stirred at room temperature for 10 min, and then 6-bromohexan-1-ol in DCM was added and the reaction mixture was stirred at room temperature overnight. TLC analysis showed formation of product. The reaction mixture was diluted with DCM, washed with water and brine. The organic layer was dried over anhydrous Na₂SO₄ and concentrated under reduced pressure to give crude product, which was purified by flash chromatography (SiO₂: 0-5% EtOAc in hexane gradient) to yield L90-6 as colorless oil (2.16 g, 70%). ¹H-NMR (300 MHz, CDCl₃) δ: 5.08 (m, 2H), 4.07 (t, J=6.6, 2H), 3.79 (t, J=7.14, 1H), 3.4 (t, J=6.84, 2H), 2.54-2.7 (m, 6H), 1.83-2.1 (m, 10H), 1.1-1.67 (m, 26H), 0.88 (m, 6H).

### Synthesis of ((4-hydroxybutyl)azanediyl)bis(hexane-6,1-diyl)bis(4,4-bis((3,7-dimethyloct-6-en-1-yl)thio)butanoate) (Compound S-1)

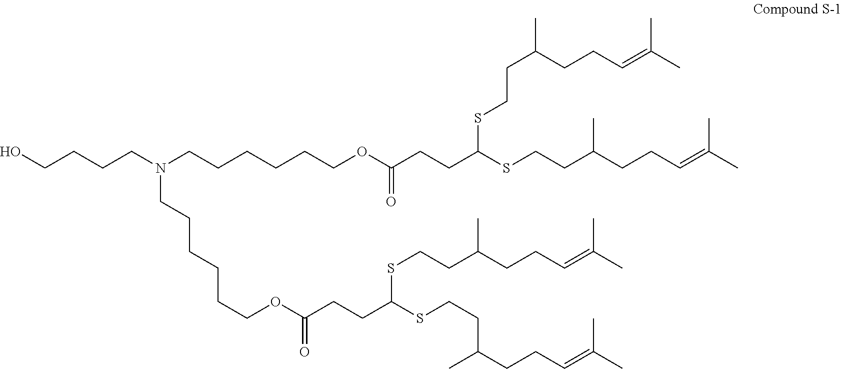 [SMILES: CC(C)=CCCC(C)CCSC(CCC(=O)OCCCCCCN(CCCCO)CCCCCCOC(=O)CCC(SCCC(C)CCC=C(C)C)SCCC(C)CCC=C(C)C)SCCC(C)CCC=C(C)C]

To a mixture of L90-6 (2.16 g, 3.65 mmol) and 4-aminobutanol (135 mg, 1.52 mmol) in ACN/CPME (1:1, 30 mL) under nitrogen was added K₂CO₃ (1.25 g, 9.12 mmol) followed by KI (504 mg, 3.04 mmol). The reaction mixture was heated at 100° C. for 40 h under nitrogen atmosphere, LC-MS analysis showed completion of the reaction. After cooling to room temperature, the reaction mixture was filtered through celite, celite cake was washed with EtOAc (2×50 mL). The filtrate was concentrated under reduced pressure to give crude product, which was purified by flash chromatography (SiO₂: 0-10% MeOH in DCM gradient) to yield Compound S-1 as colorless oil (1.06 g, 63%). ¹H-NMR (300 MHz, CDCl₃) δ: 5.08 (m, 4H), 4.04 (t, J=6.6, 4H), 3.79 (t, J=7.14, 4H), 3.55 (brs, 2H), 2.43-2.61 (m, 16H), 1.93-2.1 (m, 12H), 1.1-1.67 (s, 66H), 0.88 (m, 12H); CIMS m/z [M+H]⁺ 1110.8. Analytical HPLC column: Agela Durashell C18, 4.6×50 mmol, 3 μm (Catalog No. DC930505-0), mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=8.03 min, purity: >99.9%; UPLC column: Waters Aquity UPLC® CSH™, C18, 1.7 μm, 3.0×150 mmol, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=15.1 min, purity: 85.2%.

### Synthesis of S-5

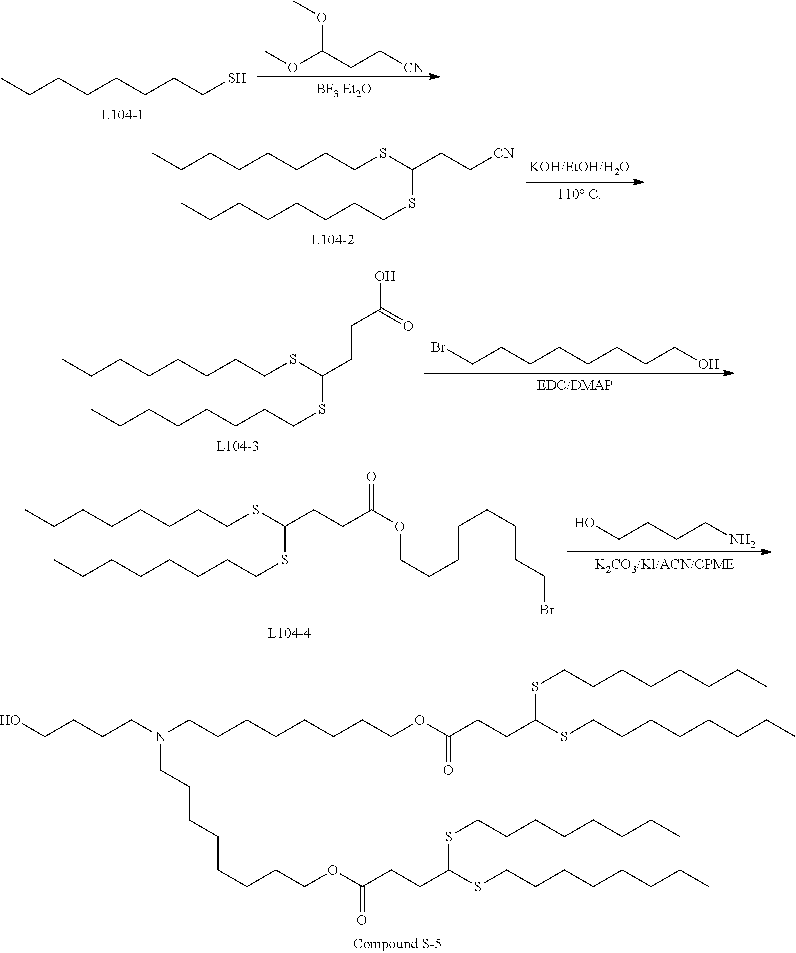

### Synthesis of 4,4-bis(octylthio) butanenitrile Intermediate L104-2

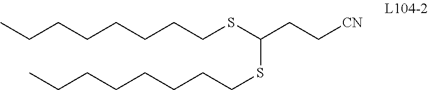 [SMILES: CCCCCCCCSC(CCC#N)SCCCCCCCC]

To a 100 mL round bottom flask 4,4-dimethoxybutanenitrile (1.0 g, 7.7 mmol) and L104-1 (3.4 g, 23.2 mmol) were taken in anhydrous dichloromethane (10 mL). The reaction was cooled in an ice water bath for 30 min. To this was added BF₃·Et₂O (219.8 mg, 1.5 mmol) and the reaction mixture was stirred at 0° C. for 2 h. The reaction was allowed to warm to room temperature and stirred for 18 h. After completion of the reaction the contents were transferred to a separatory funnel and washed twice with aqueous NaOH solution (1M, 100 mL). The organic layer was washed with water (100 mL) and brine (100 mL) and dried over anhydrous magnesium sulfate. The organic layer was concentrated to give crude product mixture. To this were added about 20 g of flash silica and dichloromethane (20 mL) and the contents were stirred well to get a uniform mixture. Solvent was removed from this mixture under vacuum. The residue was loaded on to an empty flash cartridge, which was then attached to flash purification system loaded with 40 g flash silica column and was purified by flash chromatography (SiO₂: ethyl acetate/hexane 0-10%) to get Compound L104-2 as a clear oil (2.4 g, 87%). ¹HNMR (CDCl₃) δ 3.83 (t, J=7 Hz, 1H), 2.66-2.50 (m, 6H), 2.14-2.09 (m, 2H), 1.60-1.54 (m, 4H), 1.38-1.24 (m, 20H), 0.87 (t, J=7 Hz, 6H); CIMS m/z [M+H]⁺ 358.74.

### Synthesis of 4,4-bis(octylthio) butanoic acid Intermediate L104-3

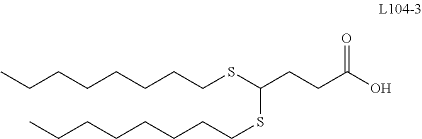 [SMILES: CCCCCCCCSC(CCC(=O)O)SCCCCCCCC]

To a 250 mL round bottom flask was added L104-2 (9.5 g, 26.56 mmol) and KOH (11.93 g, 212.5 mmol). To this was added a mixture of (1:1) Ethanol:Water (40 mL) and the reaction mixture was refluxed at 110° C. for 48 h. After completion of the reaction, the solvent was removed under vacuum. The residual paste was mixed with 100 g ice and acidified to pH 5 using 1M HCl. This mixture was partitioned in ethyl acetate:water. To this was added brine (100 mL). The ethyl acetate layer was separated and collected and the aqueous phase was extracted with ethyl acetate a further three times. The combined ethyl acetate layers were dried over anhydrous sodium sulphate. The ethyl acetate was removed under vacuum to give crude product. This crude was loaded on to a 40 g flash silica column and was purified by flash chromatography (SiO₂: ethyl acetate/hexane 0-50%) to get L104-3 as colorless oil (9.8 g, 98%). ¹HNMR (CDCl₃) δ 3.80 (t, J=7 Hz, 1H), 2.65-2.56 (m, 6H), 2.12-2.07 (m, 2H), 1.60-1.53 (m, 4H), 1.39-1.24 (m, 20H), 0.87 (t, J=7 Hz, 6H); CIMS m/z [M+H]-377.76.

### Synthesis of 8-bromooctyl 4,4-bis(octylthio)butanoate Intermediate L104-4

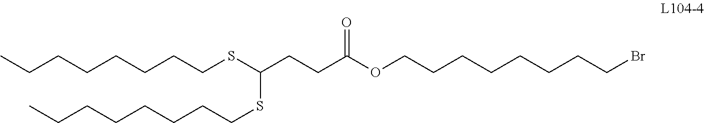 [SMILES: CCCCCCCCSC(CCC(=O)OCCCCCCCCBr)SCCCCCCCC]

To a 250 mL round bottom flask was added L104-3 (1.65 g, 4.38 mmol) and EDC (1.85 g, 9.63 mmol) in anhydrous dichloromethane (30 mL) and the reaction was stirred for 15 min. To this was added DMAP (536 mg, 4.38 mmol) and 8-bromo-1-octanol (1.37 g, 6.57 mmol) in anhydrous dichloromethane (10 mL) and the reaction mixture was stirred under nitrogen at room temperature for 18 h. After completion of the reaction, about 20 g of flash silica was added and the contents were stirred well to get a uniform mixture. Solvent was removed from this mixture under vacuum. The residue was loaded on to an empty flash cartridge, which was then attached to flash purification system loaded with 40 g flash silica column and was purified by flash chromatography (SiO₂: ethyl acetate/hexane 0-10%) to get L104-4 as a clear oil (1.3 g, 35%). ¹HNMR (CDCl₃) δ 4.08 (t, J=8 Hz, 2H), 3.79 (t, J=7 Hz, 1H), 2.65-2.24 (m, 10H), 2.12-2.07 (m, 2H), 1.65-1.25 (m, 34H), 0.89 (t, J=7 Hz, 6H); CIMS m/z [M+H]⁻ 567.76.

### Synthesis of ((4-hydroxybutyl)azanediyl)bis(octane-8,1-diyl)bis(4,4-bis(octylthio)butanoate) (S-5)

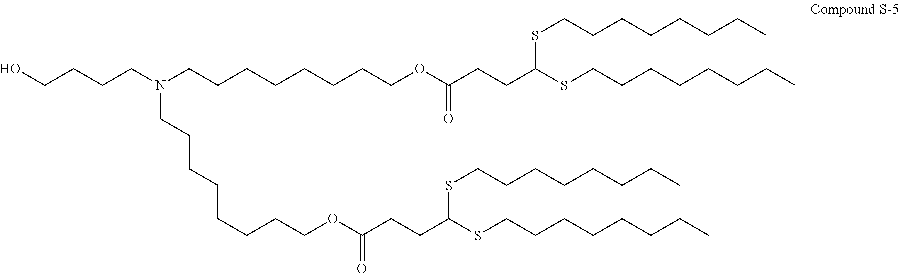 [SMILES: CCCCCCCCSC(CCC(=O)OCCCCCCCCN(CCCCO)CCCCCCCCOC(=O)CCC(SCCCCCCCC)SCCCCCCCC)SCCCCCCCC]

To a 25 mL round bottom flask was added 4-aminobutanol (160 mg, 1.79 mmol), L104-4 (2.35 g, 4.13 mmol), KI (447 mg, 2.69 mmol) and potassium carbonate (993 mg, 7.18 mmol). To this was added anhydrous ACN (2.5 mL) along with cyclopentylmethyl ether (CPME) (2.5 mL) and the reaction mixture was refluxed under nitrogen at 120° C. for 48 h. After completion of the reaction, about 20 g of silica gel (this silica was stirred in 5% triethylamine in hexane (100 mL) for 10 min before use) was added and the contents were stirred well to yield a uniform mixture. Solvent was removed from this mixture under vacuum. The residue was loaded on to an empty flash cartridge, which was then attached to a flash purification system loaded with 40 g size prepacked flash silica column (the flash column was equilibrated with 1% triethylamine in hexane for 15 min at a flow rate of 40 mL per min before use) and was purified by flash chromatography (SiO₂: ethyl acetate/hexane 0-50%) to get Compound S-5 (895 mg, 46%). ¹HNMR (CDCl₃) δ 4.06 (t, J=8 Hz, 4H), 3.79 (t, J=7 Hz, 2H), 3.56 (t, J=7 Hz, 2H), 2.65-2.39 (m, 20H), 2.12-2.07 (m, 4H), 1.65-1.25 (m, 74H), 0.87 (t, J=7 Hz, 12H); CIMS m/z [M+]⁺ 1062.9; Analytical HPLC column: Agela Durashell C18, 4.6×50 mmol, 3 μm (Catalog No. DC930505-0), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.8 min, purity: >99%; UPLC column: Waters Aquity UPLC® CSH™, C18, 1.7 μm, 3.0×150 mmol, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=16.48 min, purity: 90.2%.

### Synthesis of S-6

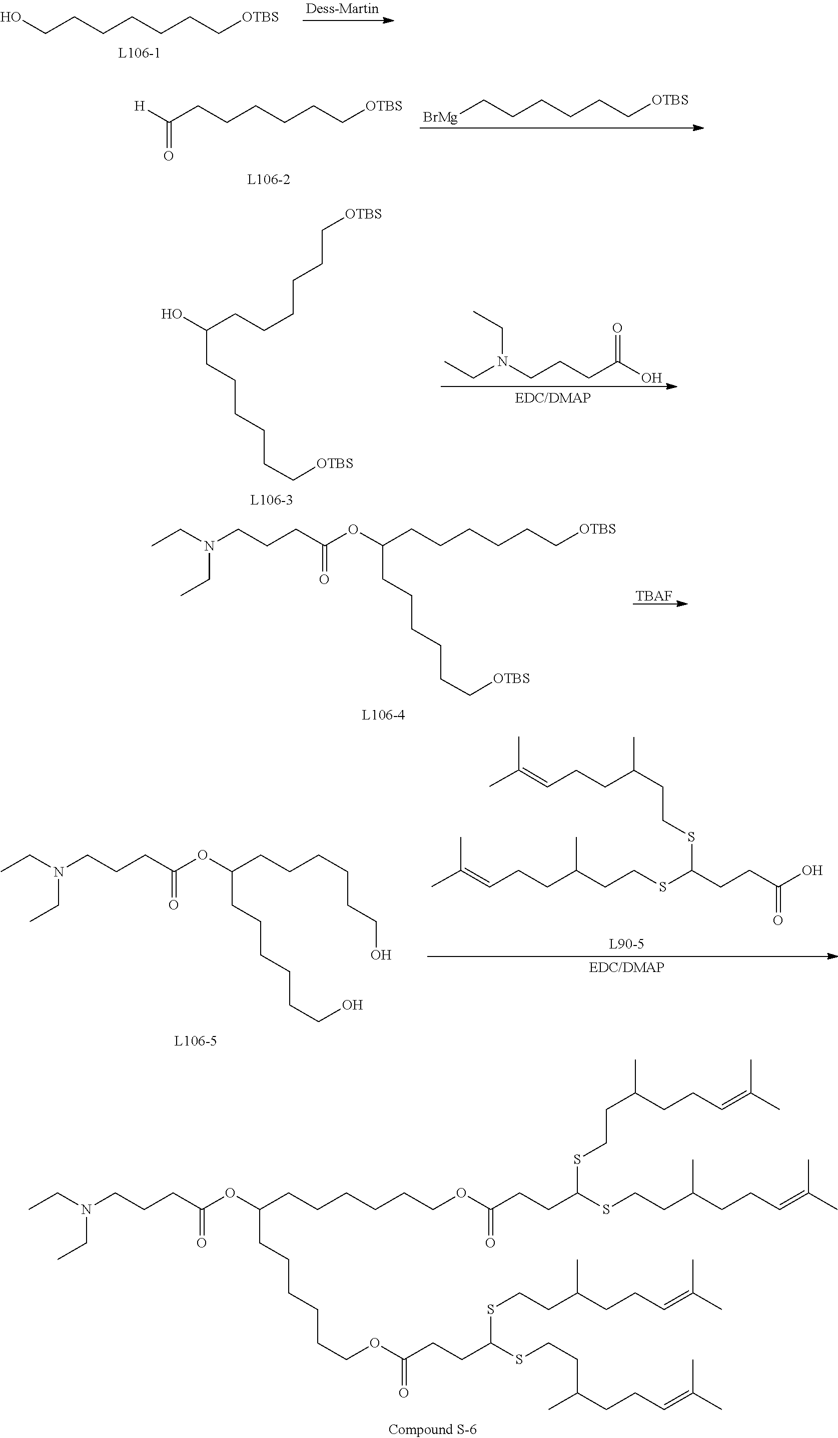

### Synthesis of 7-((tert-butyldimethylsilyl)oxy) heptanal (L106-2)

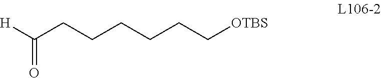 [SMILES: CC(C)(C)[Si](C)(C)OCCCCCCC=O]

L106-1 (7.6 g, 31 mmol) was dissolved in DCM (135 mL) and cooled to 0° C., followed by adding Dess-Martin periodinane (13.1 g, 31 mmol). The reaction mixture was then stirred at room temperature for 2 h. When TLC (20% EA in hexane, R_{f}=0.7) showed the completion of reaction, aq. NaOH (1N, 10 mL) was added. The quenched reaction mixture was then diluted by DCM and extracted by water twice. The organic fraction was evaporated to obtain crude product which was then subjected to silica gel column using 0-10% EA in hexane as eluent to afford L106-2 as colorless liquid (4.4 g, 60%). ¹H-NMR (300 MHz, CDCl₃) δ 9.76 (s, 1H), 3.59 (t, 2H), 2.51-2.34 (m, 2H), 1.75-1.43 (m, 4H), 1.32-1.28 (m, 4H), 0.90 (s, 9H), 0.04 (s, 6H). CIMS m/z [M+H]⁺ 245.1.

### Synthesis of 2,2,3,3,19,19,20,20-octamethyl-4,18-dioxa-3,19-disilahenicosan-11-ol (L106-3)

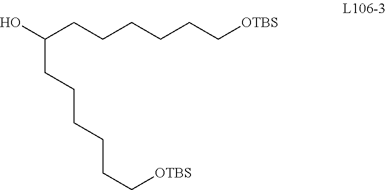 [SMILES: CC(C)(C)[Si](C)(C)OCCCCCCC(O)CCCCCCO[Si](C)(C)C(C)(C)C]

Preparation of Grignard reagent (6-((tert-butyldimethylsilyl)oxy) hexyl) magnesium bromide: Magnesium turnings (714 mg, 30 mmol) was put in a 50 mL round bottom flask and purged with N₂ for 15 min. THF (7 mL), 1,2-dibromoethane (130 μL, 1.5 mmol) were added in sequence under N₂. The reaction was then sealed and stirred for 3 min. ((6-bromohexyl)oxy) (tert-butyl)dimethylsilane (2.17 g, 7.5 mmol) was dissolved in THF (10 mL) and dropwise to the magnesium suspension. The mixture was then stirred at room temperature for 1 h and 60° C. for another hour. The dark grey solution was then cooled down to room temperature and used immediately.

To the above Grignard reagent (2.35 g, 7.5 mmol) was added L106-2 (600 mg, 2.5 mmol) in THF (7 mL). The reaction mixture was then stirred at room temperature for 2 h. When TLC (20% EA in hexane, R_{f}=0.65) showed the completion of reaction, water (10 mL) was added. The solvent was evaporated, the mixture was dissolved in EA and extracted by aq. sat. NH₄Cl. The organic fraction was evaporated to obtain crude product which was then subjected to silica gel column using 0-20% EA in hexane as eluent to afford L106-3 as colorless oil (780 mg, 69%). ¹H-NMR (300 MHz, CDCl₃) δ 3.59 (t, 3H), 1.57-1.20 (m, 20H), 0.90 (s, 18H), 0.04 (s, 12H). CIMS m/z [M+H]⁺ 461.4.

### Synthesis of 2,2,3,3,19,19,20,20-octamethyl-4,18-dioxa-3,19-disilahenicosan-11-yl 4-(diethylamino)butanoate (L106-4)

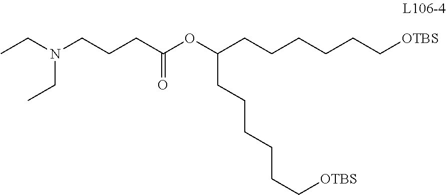 [SMILES: CCN(CC)CCCC(=O)OC(CCCCCCO[Si](C)(C)C(C)(C)C)CCCCCCO[Si](C)(C)C(C)(C)C]

The starting acid (458 mg, 2.9 mmol) was dissolved with the help of DIPEA (480 μL) in DCM (7 mL), followed by adding EDC/DMAP (1.2 g, 6.0 mmol/210 mg, 1.7 mmol). After stirring until the solution became clear, L106-3 (720 mg, 1.5 mmol) was added slowly to the above solution. The reaction mixture was stirred at room temperature for 3 h. TLC (10% MeOH in DCM with 1% NH₄OH, R_{f}=0.7) showed the completion of reaction. The solvent was evaporated to obtain crude product which was then subjected to silica gel column using 0-10% MeOH in DCM with 1% NH₄OH as eluent to afford L106-4 as colorless oil (800 mg, 85%). ¹H-NMR (300 MHz, CDCl₃) δ 4.91-4.79 (m, 1H), 3.58 (t, 4H), 2.58-2.38 (m, 6H), 2.30 (t, 2H), 1.71-1.69 (m, 2H), 1.54-1.40 (m, 8H), 1.38-1.13 (m, 12H), 1.01 (t, 6H), 0.89 (s, 18H), 0.04 (s, 12H). CIMS m/z [M+H]⁺ 602.0.

### Synthesis 1,13-dihydroxytridecan-7-yl 4-(diethylamino)butanoate (L106-5)

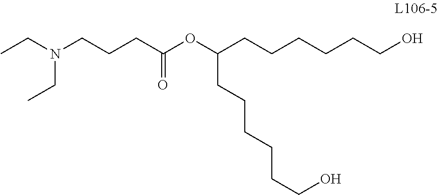 [SMILES: CCN(CC)CCCC(=O)OC(CCCCCCO)CCCCCCO]

The starting material L106-4 (800 mg, 1.3 mmol) was added to TBAF (1 M in THE, 2.5 mL). The resulting mixture was then stirred at room temperature for 20 h. When TLC (15% MeOH in DCM with 1% NH₄OH, R_{f}=0.5) showed the completion of reaction, the solvent was evaporated to obtain crude product which was then subjected to silica gel column using 0-15% MeOH in DCM with 1% NH₄OH as eluent to afford L106-5 as light yellow oil (470 mg, 95%). ¹H-NMR (300 MHz, CDCl₃) δ 4.91-4.79 (m, 1H), 3.62 (t, 4H), 2.63-2.39 (m, 6H), 2.31 (t, 2H), 1.88-1.68 (m, 2H), 1.62-1.41 (m, 8H), 1.40-1.19 (m, 12H), 1.05 (t, 6H). CIMS m/z [M+H]⁺ 374.3.

### Synthesis of 7-((4-(diethylamino)butanoyl)oxy)tridecane-1,13-diyl bis(4,4-bis((3,7-dimethyloct-6-en-1-yl)thio)butanoate) (Compound S-6)

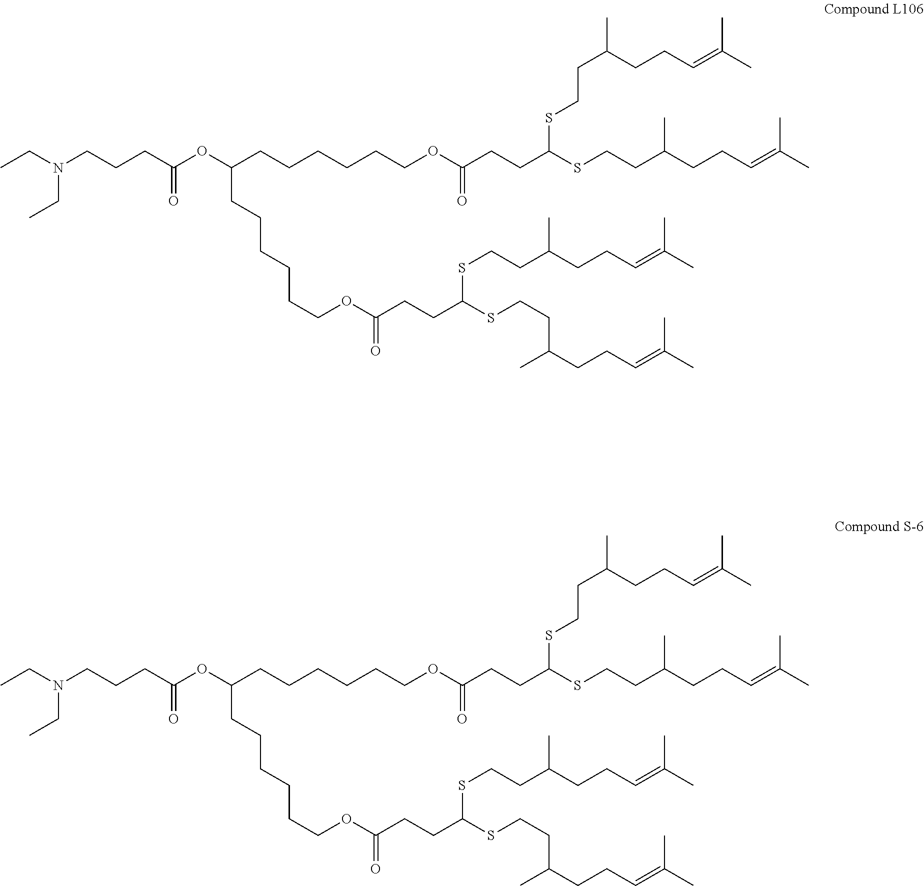

The starting material acid (934 mg, 2.2 mmol) was dissolved in DCM (30 mL), followed by adding EDC/DMAP (1.53 g, 8 mmol/266 mg, 2.2 mmol). After stirring 5 minutes until the solution was clear then added L106-5 (370 mg, 1.0 mmol) slowly to the above solution, the reaction mixture was stirred for 3 h at room temperature till L106-5 disappeared on TLC. When TLC (10% MeOH in DCM with 1% NH₄OH, R_{f}=0.6) showed the completion of reaction, and the organic fraction was evaporated to obtain crude product which was then subjected to silica gel column using 0-10% MeOH in DCM with 1% NH₄OH as eluent to afford Compound S-6 as light-yellow oil (1.0 g, 85%). ¹H-NMR (300 MHz, CDCl₃) δ 5.15-5.01 (m, 4H), 4.85 (quint, 1H), 4.04 (t, 4H), 3.79 (t, 2H), 2.76-2.42 (m, 18H), 2.32 (t, 2H), 2.10 (q, 4H), 1.96 (quint, 8H), 1.72-1.46 (m, 40H), 1.45-1.24 (m, 22H), 1.20-1.08 (m, 4H), 1.04 (bs, 6H), 0.88 (d, 12H). CIMS m/z [M+H]⁺ 1194.8. Analytical HPLC column: Agela Durashell C18, 4.6×50 mmol, 3 μm (Catalog No. DC930505-0), mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=7.06 min, purity: 98.7%; UPLC column: Waters Aquity UPLC® CSH™, C18, 1.7 μm, 3.0×150 mmol, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=7.50 min, purity: 90.2%.

### Synthesis of S-7

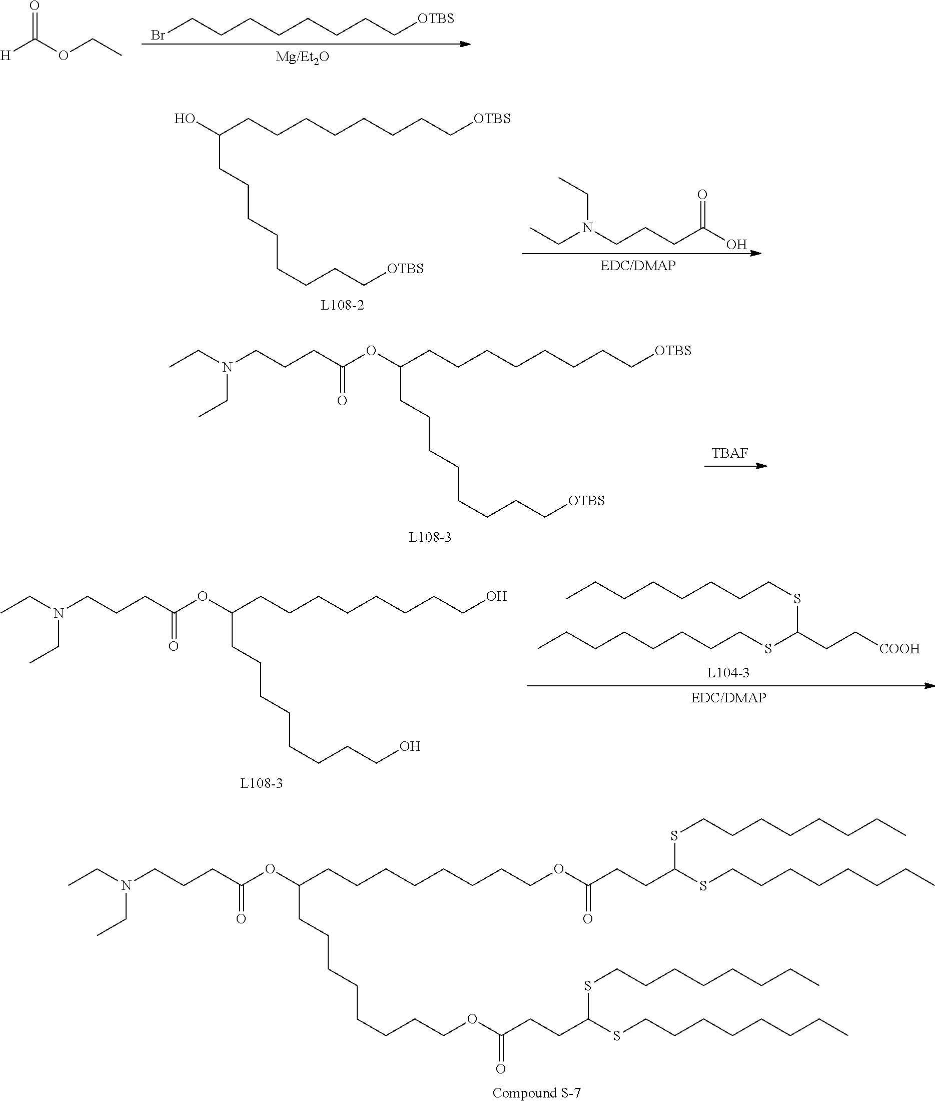

### Synthesis of 2,2,3,3,23,23,24,24-octamethyl-4,22-dioxa-3,23-disilapentacosan-13-ol (L108-2)

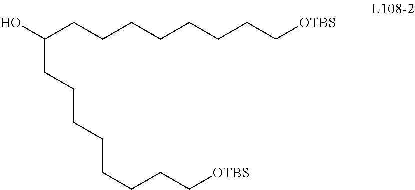 [SMILES: CC(C)(C)[Si](C)(C)OCCCCCCCCC(O)CCCCCCCCO[Si](C)(C)C(C)(C)C]

To an oven dried 1000 mL three neck round bottom flask equipped with magnetic stir bar and fitted with a reflux condenser, magnesium turnings (4.84 g, 202.48 mmol) and one tiny crystal of iodine were added, followed by addition of anhydrous THF (50 mL). The reaction mixture was allowed to stir for 30 min till iodine color faded. To this was added 1,2-dibromoethane (0.6 mL) and stirring was continued for 60 min. (8-bromooctyl)oxy) (tert-butyl)dimethylsilane (8.72 g, 26.97 mmol) was dissolved in anhydrous THF (50 mL) and added dropwise to the reaction. After completion of addition, the reaction was refluxed for 2 h then allowed to cool to room temperature, followed by cooling in ice-water bath for 30 min. To this reaction was added L108-1 (600 mg, 8.09 mmol) and the reaction was stirred at room temperature for 18 h. The reaction was quenched using aq. sat. ammonium chloride solution (300 mL) followed by partitioning in ethyl acetate: water. Brine (200 mL) was added, and the mixture was filtered through celite to break the emulsion formation. The aqueous phase was extracted with ethyl acetate three times. The combined ethyl acetate layers were dried over anhydrous sodium sulphate. The ethyl acetate was removed under vacuum to give crude product. This crude was loaded on to 220 g flash silica column and was purified by flash chromatography (SiO₂: ethyl acetate/hexane 0-50%) to get Compound L108-2 as colorless oil (4.18 g, 87%). ¹H-NMR (300 MHz, CDCl₃) δ: 3.58 (t, J=8.0 Hz, 5H), 1.57-1.25 (m, with sharp singlet at 1.29, 29H), 0.89 (s, 18H), 0.04 (s, 12H); CIMS m/z [M+H]⁺ 518.0.

### Synthesis of 2,2,3,3,23,23,24,24-octamethyl-4,22-dioxa-3,23-disilapentacosan-13-yl 4-(diethylamino)butanoate (L108-3)

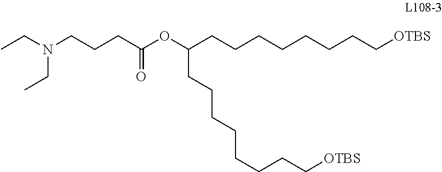 [SMILES: CCN(CC)CCCC(=O)OC(CCCCCCCCO[Si](C)(C)C(C)(C)C)CCCCCCCCO[Si](C)(C)C(C)(C)C]

To a 250 mL round bottom flask the starting acid (2.63 g, 13.4 mmol), EDC (5.45 g, 28.43 mmol) and DMAP (1.98 g, 16.25 mmol) were taken in anhydrous dichloromethane (30 mL) and the reaction was stirred for 15 min. To this was added N, N-Diisopropylethylamine (6.01 mL, 34.53 mmol) along with L108-2 (2.1 g, 4.06 mmol) in anhydrous dichloromethane (10 mL) and the reaction mixture was stirred under nitrogen at room temperature for 48 h. After completion of reaction about 20 g of flash silica (neutralized with triethylamine) was added and the contents were stirred well to get a uniform mixture. Solvent was removed from this mixture under vacuum. The residue was loaded on to an empty flash cartridge, which was then attached to flash purification system loaded with 40 g flash silica column (equilibrated with 1% triethylamine in hexane) and was purified by flash chromatography (SiO₂: ethyl acetate/hexane (with 1% triethylamine) 0-10%) to get L108-3 as a clear oil (2.67 g, 96%). ¹H-NMR (300 MHz, CDCl₃) δ: 4.85 (t, J=7.0 Hz, 1H), 3.58 (t, J=7.0 Hz, 4H), 2.61-2.40 (m, 8H), 1.81-1.71 (m, 2H), 1.62-1.25 (m, with sharp singlet at 1.26, 28H), 0.98 (t, J=7.0 Hz, 6H), 0.89 (s, 18H), 0.04 (s, 12H); CIMS m/z [M+H]⁺ 659.29.

### Synthesis of 1,17-dihydroxyheptadecan-9-yl 4-(diethylamino)butanoate (Compound L108-4)

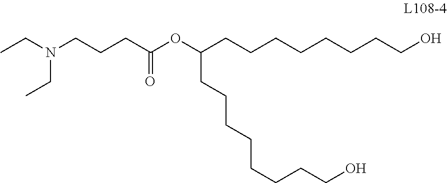 [SMILES: CCN(CC)CCCC(=O)OC(CCCCCCCCO)CCCCCCCCO]

To a 100 mL round bottom flask L108-3 (2.5 g, 3.66 mmol) and TBAF (3.67 g, 13.5 mmol) were taken. To this was added anhydrous THF (10 mL) and the reaction mixture was stirred at room temperature under inert conditions for 24 h. After completion of the reaction the solvent was removed under vacuum. Residue paste was loaded on to a prepacked flash cartridge, which was then attached to flash purification system loaded with 40 g flash silica column and was purified by flash chromatography (0-10% dichloromethane:methanol) to get L108-4 as a clear oil (1.34 g, 82%). ¹H-NMR (300 MHz, CDCl₃) δ: 4.87 (t, J=7.0 Hz, 1H), 3.62 (t, J=7.0 Hz, 4H), 3.41-3.29 (m, 6H), 2.61-2.40 (bs, 2H), 1.94 (t, J=7.0 Hz, 2H), 1.82-1.25 (m, with sharp singlet at 1.27, 24H), 0.98-0.96 (m, 6H), 0.92 (t, J=7.0 Hz, 6H); CIMS m/z [M+H]⁺ 430.6.

### Synthesis of 9-((4-(diethylamino)butanoyl)oxy) heptadecane-1,17-diyl bis(4,4-bis(octylthio) butanoate) (Compound S-7)

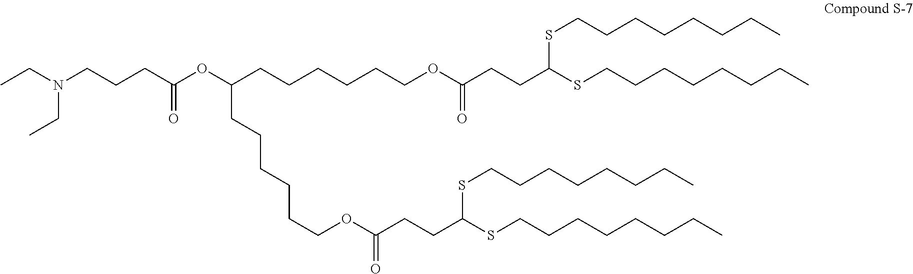 [SMILES: CCCCCCCCSC(CCC(=O)OCCCCCCC(CCCCCCOC(=O)CCC(SCCCCCCCC)SCCCCCCCC)OC(=O)CCCN(CC)CC)SCCCCCCCC]

To a 250 mL round bottom flask the starting acetal acid L104-3 (2.3 g, 6.19 mmol), EDC (3.12 g, 16.29 mmol) and DMAP (995 mg, 8.14 mmol) were taken in anhydrous dichloromethane (20 mL) and the reaction was stirred for 5 min. To this was added L108-4 (700 mg, 1.63 mmol) in anhydrous dichloromethane (10 mL) and the reaction mixture was stirred under nitrogen at room temperature for 48 h. After completion of reaction about 30 g of flash silica (neutralized with triethylamine) was added and the contents were stirred well to get a uniform mixture. Solvent was removed under vacuum. The residue was loaded on to an empty flash cartridge, which was then attached to flash purification system loaded with 80 g flash silica column (equilibrated with 1% triethylamine in hexane) and was purified by flash chromatography (SiO₂; ethyl acetate/hexane (with 1% triethylamine) 0-20%) to get Compound S-7 as a clear oil (1.1 g, 63%). ¹H-NMR (300 MHz, CDCl₃) δ: 4.87 (t, J=7.0 Hz, 1H), 4.04 (t, J=7.0 Hz, 4H), 3.62 (1, J=7.0 Hz, 2H), 2.60-2.35 (m, 20H), 2.30 (t, J=7.0 Hz, 2H), 2.09 (t, J=7.0 Hz, 4H), 1.79-1.65 (m, 2H), 1.59-1.25 (m, with sharp singlet at 1.27, 74H), 1.02 (t, J=7.0 Hz, 6H), 0.86 (t, J=7.0 Hz, 12H); CIMS m/z [M+H]⁺ 1147.98 Analytical HPLC column: Agela Durashell C18, 4.6×50 mmol, 3 μm (Catalog No. DC930505-0), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.4 min, purity: >99%; UPLC column: Waters Aquity UPLC® CSH™, C18, 1.7 μm, 3.0×150 mmol, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=16.6 min, purity: >99%.

### Synthesis of CC-1

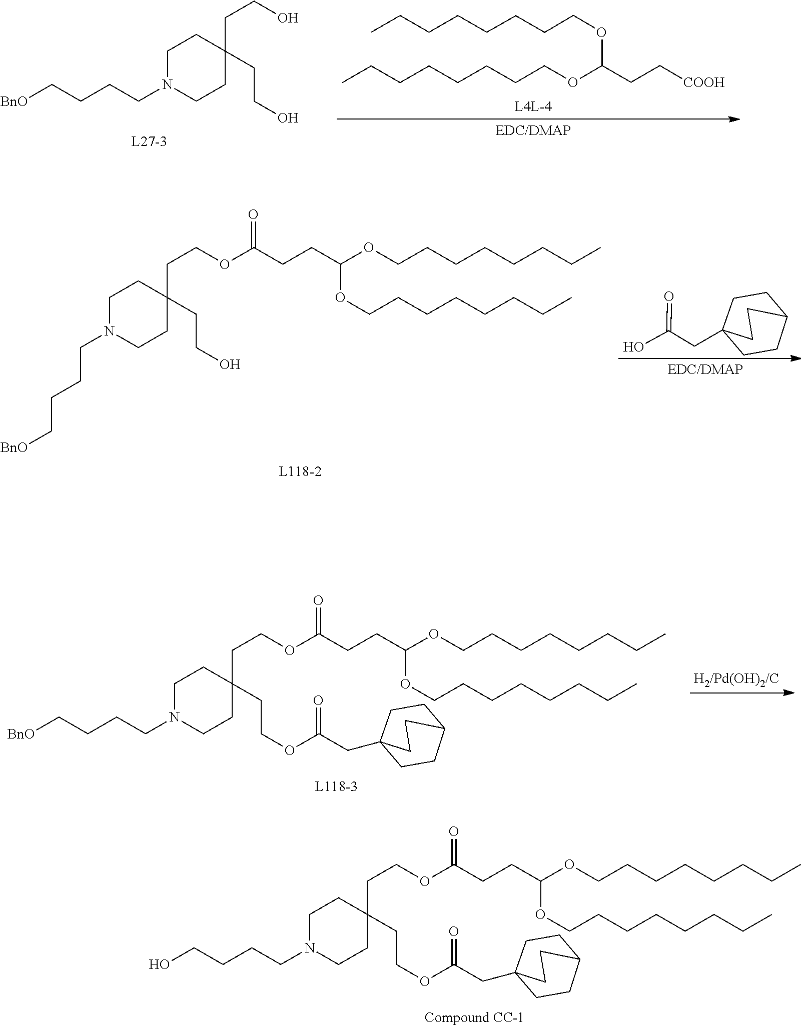 [SMILES: OCCC1(CCO)CCN(CCCCOCc2ccccc2)CC1 ; CCCCCCCCOC(CCC(=O)O)OCCCCCCCC ; CCCCCCCCOC(CCC(=O)OCCC1(CCO)CCN(CCCCOCc2ccccc2)CC1)OCCCCCCCC ; O=C(O)CC12CCC(CC1)CC2 ; CCCCCCCCOC(CCC(=O)OCCC1(CCOC(=O)CC23CCC(CC2)CC3)CCN(CCCCOCc2ccccc2)CC1)OCCCCCCCC ; CCCCCCCCOC(CCC(=O)OCCC1(CCOC(=O)CC23CCC(CC2)CC3)CCN(CCCCO)CC1)OCCCCCCCC]

### Synthesis of 2-(1-(4-(benzyloxy)butyl)-4-(2-hydroxyethyl) piperidin-4-yl)ethyl 4,4-bis(octyloxy)butanoate (L118-2)

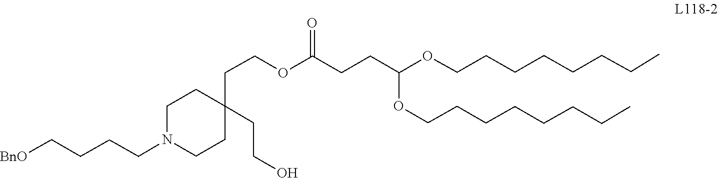 [SMILES: CCCCCCCCOC(CCC(=O)OCCC1(CCO)CCN(CCCCOCc2ccccc2)CC1)OCCCCCCCC]

A solution of L4L-4 (1 g, 4.38 mmol) and EDC/DMAP (2.23 g, 17.53 mmol/390 mg, 4.82 mmol) in DCM (74 mL) was stirred for 5 minutes at room temperature to form a clear solution. The L4L-4 solution was then added dropwise over 1 hour to a solution of L27-3 (1.47 g, 4.38 mmol) in DCM (147 mL), and the reaction mixture was stirred for 2 an additional hours at room temperature. When TLC (15% MeOH in DCM with 1% NH₄OH, R_{f}=0.6) showed the completion of reaction, the solvent was evaporated. The obtained crude product was then subjected to silica gel column twice using 0-15% MeOH in DCM with 1% NH₄OH as eluent to afford L118-2 as light-yellow oil (1.07 g, 56%). ¹H-NMR (300 MHz, CDCl₃) δ 7.34 (m, 5H), 4.45 (m, 3H), 4.12 (t, 2H), 3.70 (t, 2H), 3.61-3.34 (m, 6H), 2.50-2.29 (m, 4H), 1.92 (q, 2H), 1.72-1.41 (m, 12H), 1.38-1.12 (m, 24H), 0.85 (t, 6H).

### Synthesis of 2-(1-(4-(benzyloxy)butyl)-4-(2-(2-(bicyclo[2.2.2]octan-1-yl) acetoxy)ethyl)piperidin-4-yl)ethyl 4,4-bis(octyloxy)butanoate (L118-3)

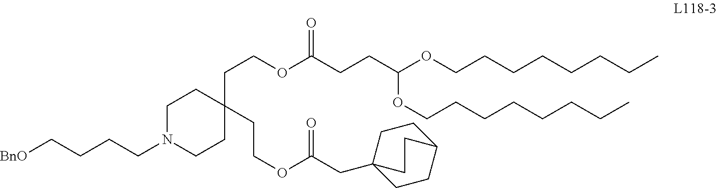 [SMILES: CCCCCCCCOC(CCC(=O)OCCC1(CCOC(=O)CC23CCC(CC2)CC3)CCN(CCCCOCc2ccccc2)CC1)OCCCCCCCC]

2-(bicyclo[2.2.2]octan-1-yl) acetic acid (153 mg, 0.91 mmol) and EDC/DMAP (696 mg, 3.64 mmol/120 mg, 0.27 mmol) were dissolved in DCM (20 mL). After stirring for 5 minutes the solution was clear. L118-2 (500 mg, 0.208 mmol) was then added to the above solution, and the reaction mixture was stirred for 3 hours at room temperature. When TLC (12% MeOH in DCM, R_{f}=0.8) showed the completion of reaction, the solvent was evaporated. The obtained crude product was then subjected to silica gel column twice using 0-15% MeOH in DCM as eluent to afford L118-3 as light-yellow oil (570 mg, 93%). ¹H-NMR (300 MHz, CDCl₃) δ 7.34 (m, 5H), 4.48 (m, 3H), 4.14-4.01 (m, 4H), 3.62-3.31 (m, 6H), 2.50-2.29 (m, 6H), 2.01 (s, 2H), 1.92 (q, 2H), 1.78-1.41 (m, 25H), 1.38-1.02 (m, 20H), 0.85 (t, 6H). CIMS m/z [M+H]⁺ 812.5.

### Synthesis of 2-(4-(2-(2-(bicyclo[2.2.2]octan-1-yl)acetoxy)ethyl)-1-(4-hydroxybutyl) piperidin-4-yl)ethyl 4,4-bis(octyloxy)butanoate (Compound CC-1)

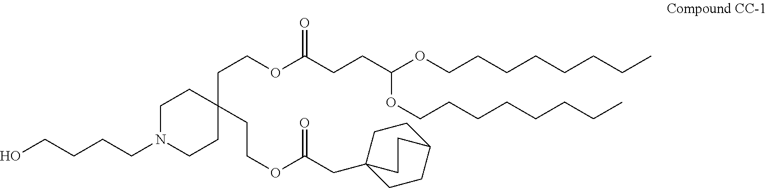 [SMILES: CCCCCCCCOC(CCC(=O)OCCC1(CCOC(=O)CC23CCC(CC2)CC3)CCN(CCCCO)CC1)OCCCCCCCC]

L118-3 (570 mg, 0.86 mmol) was dissolved in MeOH (20 mL), followed by addition of 20 wt % Pd (OH) 2/C (300 mg). The mixture was stirred under H₂ atmosphere at room temperature for 3 hours. When TLC (15% MeOH in DCM with 1% NH₄OH, R_{f}=0.8) showed the completion of reaction, the solvent was evaporated to obtain crude product which was then subjected to silica gel column using 0-15% MeOH in DCM with 1% NH₄OH as eluent to afford Compound CC-1 as yellow oil (330 mg, 65%). ¹H-NMR (300 MHz, CDCl₃) δ 4.48 (1, 1H), 4.14-4.01 (m, 4H), 3.62-3.48 (m, 4H), 3.44-3.32 (m, 2H), 2.62-2.29 (m, 8H), 2.01 (s, 2H), 1.92 (q, 2H), 1.78-1.41 (m, 30H), 1.38-1.02 (m, 24H), 0.85 (t, 6H); CIMS m/z [M+H]⁺ 722.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=5.75 min, purity: >99.9%; UPLC column: Waters Aquity UPLC® CSH™, C18, 1.7 μm, 3.0×150 mmol, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=7.74 min, purity: 97.5%.

### Synthesis of CC-2

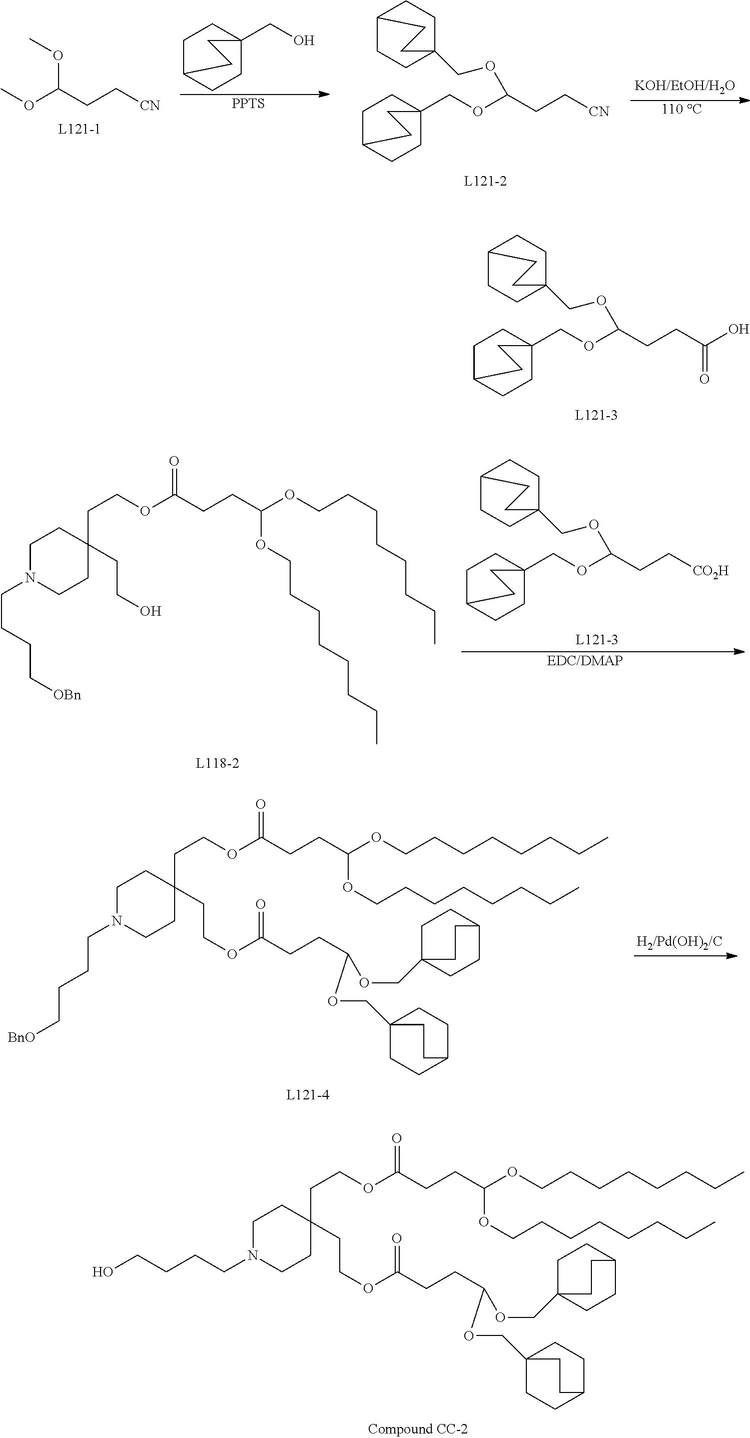

### Synthesis of 4,4-bis(bicyclo[2.2.2]octan-1-ylmethoxy) butanenitrile (L121-2)

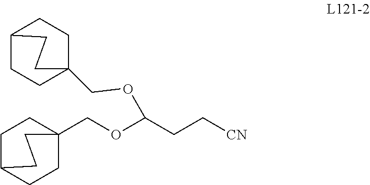 [SMILES: N#CCCC(OCC12CCC(CC1)CC2)OCC12CCC(CC1)CC2]

The starting materials L121-1 (170 mg, 1.32 mmol), bicyclo[2.2.2]octan-1-ylmethanol (500 mg, 2.90 mmol) and p-toluenesulfonic acid (50 mg, 0.13 mmol) were dissolved in DMF (1 mL). The reaction mixture was then heated to 110° C. and stirred for 3 hours. When TLC (20% EA in hexane, R_{f}=0.8) showed the completion of reaction, the reaction was cooled to room temperature. Solvent was evaporated to obtain crude product which was subjected to silica gel column using 0-20% EA in hexane as eluent to afford L121-2 as white solid (377 mg, 84%). ¹H-NMR (300 MHz, CDCl₃) δ 4.43 (t, 1H), 3.28-3.04 (m, 2H), 2.98-2.73 (m, 2H), 2.39 (t, 2H), 1.92 (t, 2H), 1.68-1.41 (m, 13H), 1.40-1.02 (m, 13H).

### Synthesis of 4,4-bis(bicyclo[2.2.2]octan-1-ylmethoxy) butanoic acid (L121-3)

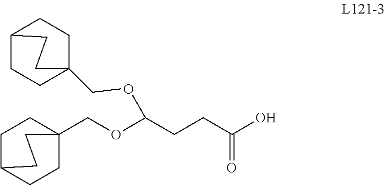 [SMILES: O=C(O)CCC(OCC12CCC(CC1)CC2)OCC12CCC(CC1)CC2]

L121-2 (377 mg, 1.09 mmol) was dissolved in EtOH (3.5 mL) and added to a 30 mL-pressure vial. KOH (196 mg, 3.49 mmol) was dissolved in water (3.5 mL) and then added to the above solution. This mixture was then sealed and stirred at 110° C. for 16 hours while the reaction turned from a cloudy to clear solution. EtOH was then evaporated. The remaining aqueous portion was diluted by water (10 mL), acidified with 1 M HCl until pH=4 and extracted by EA (20 mL×2). The organic fraction was evaporated to obtain product L121-3 as white solid (360 mg, 91%). ¹H-NMR (300 MHz, CDCl₃) δ 4.40 (t, 1H), 3.21-3.04 (m, 2H), 2.98-2.80 (m, 2H), 2.42 (t, 2H), 1.98-1.81 (m, 2H), 1.68-1.41 (m, 13H), 1.40-1.02 (m, 13H).

### Synthesis of 2-(1-(4-(benzyloxy)butyl)-4-(2-((4,4-bis(bicyclo[2.2.2]octan-1-ylmethoxy) butanoyl)oxy)ethyl)piperidin-4-yl)ethyl 4,4-bis(octyloxy)butanoate (L121-4)

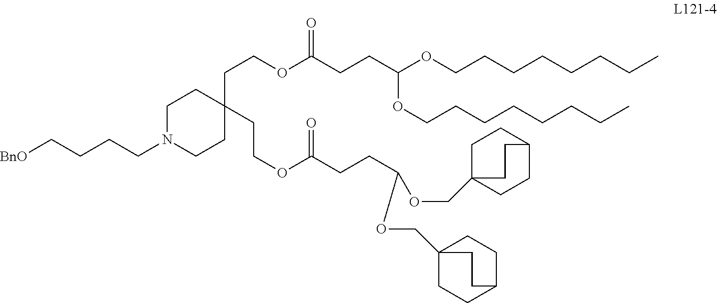 [SMILES: CCCCCCCCOC(CCC(=O)OCCC1(CCOC(=O)CCC(OCC23CCC(CC2)CC3)OCC23CCC(CC2)CC3)CCN(CCCCOCc2ccccc2)CC1)OCCCCCCCC]

L121-3 (360 mg, 0.99 mmol) and EDC/DMAP (760 mg, 3.95 mmol/130 mg, 1.09 mmol) were dissolved in DCM (20 mL). After stirring for 5 minutes the solution became clear. L118-2 (440 mg, 0.66 mmol; see synthesis of CC-1) was then added to the above solution. The reaction mixture was stirred for 3 hours at room temperature. When TLC (12% MeOH in DCM, R_{f}=0.8) showed the completion of reaction, the solvent was evaporated. The crude product was subjected to silica gel column twice using 0-15% MeOH in DCM as eluent to afford L121-4 as light-yellow oil (650 mg, 97%). ¹H-NMR (300 MHz, CDCl₃) δ 7.37-7.28 (m, 5H), 4.50-4.48 (m, 2H), 4.38 (t, 1H), 4.11 (t, 4H), 3.58-3.31 (m, 6H), 3.21-3.04 (m, 2H), 2.98-2.80 (m, 2H), 2.51-2.20 (m, 8H), 1.98-1.77 (m, 4H), 1.73-1.32 (m, 30H), 1.32-1.05 (m, 33H), 0.87 (t, 6H); CIMS m/z [M+H]⁺ 1008.7.

### Synthesis of 2-(4-(2-((4,4-bis(bicyclo[2.2.2]octan-1-ylmethoxy) butanoyl)oxy)ethyl)-1-(4-hydroxybutyl) piperidin-4-yl)ethyl 4,4-bis(octyloxy)butanoate (Compound CC-2)

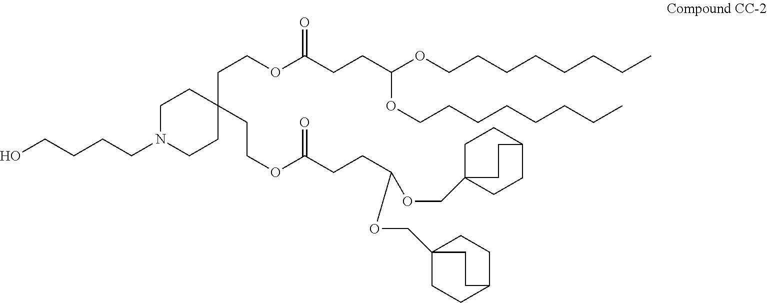 [SMILES: CCCCCCCCOC(CCC(=O)OCCC1(CCOC(=O)CCC(OCC23CCC(CC2)CC3)OCC23CCC(CC2)CC3)CCN(CCCCO)CC1)OCCCCCCCC]

L121-4 (650 mg, 0.64 mmol) was dissolved in MeOH/DCM (20/5 mL), followed by addition of 20 wt % Pd (OH) 2/C (350 mg). The mixture was stirred under H₂ atmosphere at room temperature for 3 hours. When TLC (15% MeOH in DCM with 1% NH₄OH, R_{f}=0.8) showed the completion of reaction, the solvent was evaporated to obtain crude product which was then subjected to silica gel column using 0-15% MeOH in DCM with 1% NH₄OH as eluent to give Compound CC-2 as yellow oil (360 mg, 61%). ¹H-NMR (300 MHz, CDCl₃) δ 4.48 (t, 1H), 4.37 (t, 1H), 4.11 (t, 4H), 3.64-3.48 (m, 4H), 3.44-3.26 (m, 3H), 3.18-3.08 (m, 2H), 2.94-2.86 (m, 2H), 2.74-2.46 (m, 6H), 2.40-2.28 (m, 5H), 1.96-1.82 (m, 4H), 1.78-1.60 (m, 10H), 1.60-1.42 (m, 22H), 1.38-1.04 (m, 40H), 0.87 (t, 6H). CIMS m/z [M+H]⁺ 918.7. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=6.96 min, purity: 97.7%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=6.72 min, purity: 98.7%.

### Synthesis of CC-100

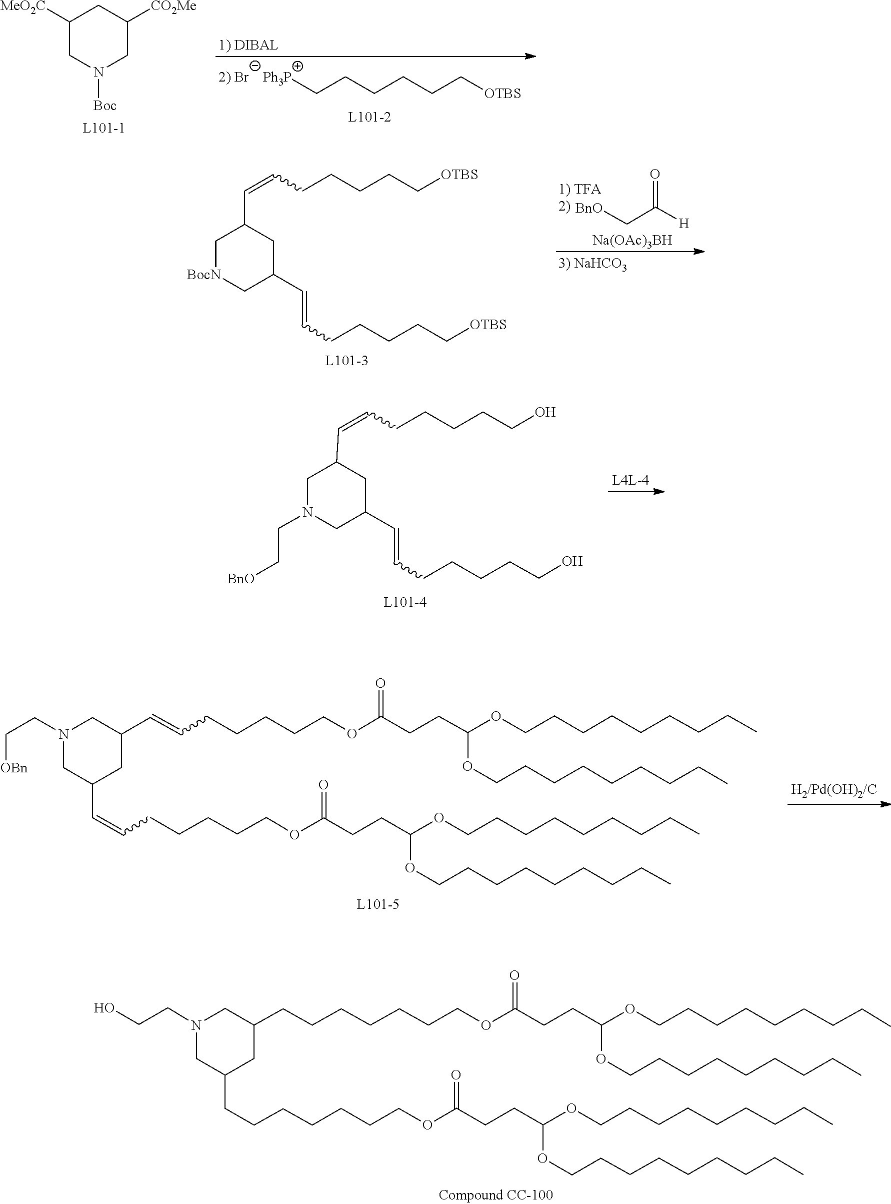

### Synthesis of tert-butyl 3,5-bis(7-((tert-butyldimethylsilyl)oxy) hept-1-en-1-yl) piperidine-1-carboxylate (L101-3)

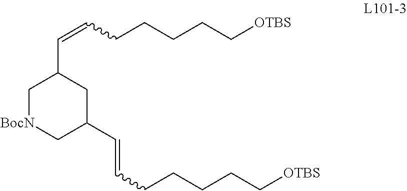 [SMILES: CC(C)(C)OC(=O)N1CC(C=CCCCCCO[Si](C)(C)C(C)(C)C)CC(C=CCCCCCO[Si](C)(C)C(C)(C)C)C1]

To a dry ice-acetone bath cooled solution of L101-1 (500 mg, 1.6 mmol) in anhydrous toluene (20 mL) was added 1.0 M diisobutylaluminum hydride in toluene (3.4 mL, 3.4 mmol) under nitrogen atmosphere. The resulting mixture was stirred at −72° C. for 2 h. About half of a pre-cooled (−72° C.) solution of benzyloxypropylidene triphenylphosphorane (obtained by adding potassium tert-butoxide (1.05 g, 10 mmol) to a solution of (3-benzyloxypropyl)tripheny phosphonium bromide L101-2 (5.5 g, 11 mmol) in anhydrous toluene (8 mL) at 0° C., and then stirred at room temperature for 2 h). The reaction mixture was warmed to room temperature and stirred for 16 h. The rest of the solution of Wittig reagent was added, and the reaction was stirred at room temperature for another 30 h. The reaction was then quenched by adding water (15 mL) and extracted with ethyl acetate (25 mL×3). Combined organic extracts were washed with water (25 mL×3) and dried over anhydrous Na₂SO₄. Filtration and concentration provided crude material which was purified by flash column chromatography (SiO₂: ethyl acetate/hexane 0-100) to yield L101-3 as colorless oil (329 mg, 30%). ¹H-NMR (300 MHz, CDCl₃) δ 5.42-5.37 (m, 2H), 5.09-5.05 (m, 2H), 3.97 (s, br, 2H), 3.59 (t, J=6.6 Hz, 4H), 2.48-1.95 (m, 8H), 1.79-1.23 (m, 14H), 1.45 (s, 9H), 0.89 (s, 18H), 0.05 (s, 12H); CIMS m/z [M-Boc+H]⁺ 538.

### Synthesis of 7,7′-(1-(2-(benzyloxy)ethyl) piperidine-3,5-diyl)bis(hept-6-en-1-ol) (L101-4)

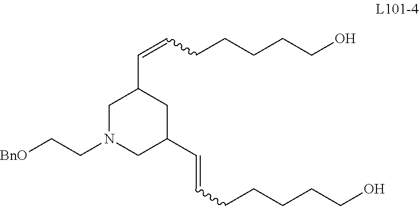 [SMILES: OCCCCCC=CC1CC(C=CCCCCCO)CN(CCOCc2ccccc2)C1]

To a solution of L101-3 (200 mg, 0.31 mmol) in dichloromethane (1.5 mL) was added TFA (1.5 mL) at 0° C. and the reaction mixture was stirred at room temperature for 4 h. The volatile components were removed under reduced pressure. The crude was dissolved in 1,2-dichloroethane (8 mL) and mixed with a solution of 4-benzyloxyacetal (94 mg, 0.62 mmol) in 1,2-dichloroethane (8 mL). Sodium triacetoxyborohydride (198 mg, 0.93 mmol) was added followed by acetic acid (40 μL, 0.62 mmol). The resulting mixture was stirred at room temperature under nitrogen atmosphere for 20 h and then concentrated. To the residue was added sat. aq. sodium bicarbonate solution (2 mL) and methanol (6 mL). The reaction mixture was stirred at room temperature for 12 h. After neutralizing with 0.1 N HCl, the reaction mixture was concentrated under reduced pressure. The residue was mixed with water (5 mL) and extracted with DCM (10 mL×3). Combined organic extracts were dried over anhydrous Na₂SO₄. Filtration and concentration provided crude material which was purified by flash column chromatography (SiO₂: ethyl acetate/hexane 0-100% with 1% triethylamine in the eluent) to yield L101-4 as slightly yellow oil (70 mg, 40%). ¹H-NMR (300 MHz, CDCl₃) δ 7.36-7.18 (m, 5H), 5.46-5.32 (m, 2H), 5.15-4.98 (m, 2H), 4.53 (s, 2H), 3.65-3.59 (m, 6H), 2.90-2.55 (m, 6H), 2.12-1.90 (m, 6H), 1.79-1.45 (m, 6H), 1.44-1.20 (m, 8H); CIMS m/z [M+H]⁺ 444.

### Synthesis of (1-(2-(benzyloxy)ethyl) piperidine-3,5-diyl)bis(hept-6-ene-7,1-diyl)bis(4,4-bis(nonyloxy)butanoate) (L101-5)

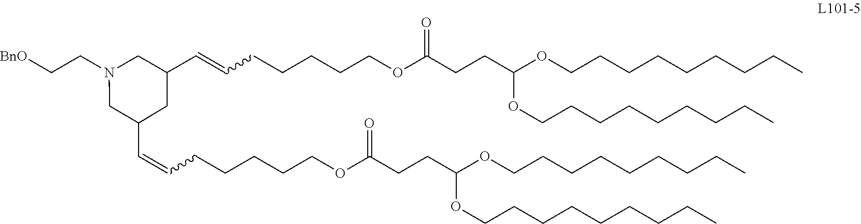 [SMILES: CCCCCCCCCOC(CCC(=O)OCCCCCC=CC1CC(C=CCCCCCOC(=O)CCC(OCCCCCCCCC)OCCCCCCCCC)CN(CCOCc2ccccc2)C1)OCCCCCCCCC]

To a solution of L101-4 (70 mg, 0.16 mmol) in DCM (5 mL) was added L4L-4 (155 mg, 0.39 mmol) followed by DMAP (22 mg, 0.18 mmol) and EDC (126 mg, 0.64 mmol). The resulting mixture was stirred at room temperature under nitrogen atmosphere for 20 h. The reaction mixture was diluted with DCM (10 mL) and washed with brine (10 mL). The organic phase was dried over anhydrous Na₂SO₄. Filtration and concentration provided crude material which was purified by flash column chromatography (SiO₂: ethyl acetate/hexane 0-100%) to yield L101-5 as colorless oil (101 mg, 56%). 1H-NMR (300 MHz, CDCl₃) δ 7.36-7.18 (m, 5H), 5.38-5.28 (m, 2H), 5.15-5.06 (m, 2H), 4.53 (s, 2H), 4.49 (t, J=5.5 Hz, 2H), 4.04 (t, J=6.6 Hz, 4H), 3.61-3.32 (m, 10H), 2.80-2.56 (m, 6H), 2.34 (t, J=7.4 Hz, 4H), 2.12-1.88 (m, 6H), 1.70-1.43 (m, 16H), 1.43-1.12 (m, 60H), 0.88 (t, J=7.1 Hz, 12H); CIMS m/z [M-Boc+H]⁺ 1152.9.

### Synthesis of (1-(2-hydroxyethyl) piperidine-3,5-diyl)bis(heptane-7,1-diyl)bis(4,4-bis(nonyloxy)butanoate) (Compound CC-100)

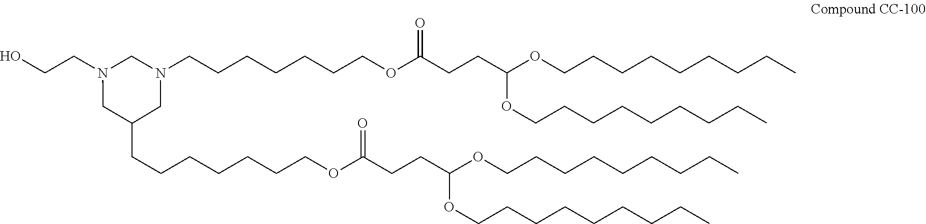 [SMILES: CCCCCCCCCOC(CCC(=O)OCCCCCCCC1CN(CCO)CN(CCCCCCCOC(=O)CCC(OCCCCCCCCC)OCCCCCCCCC)C1)OCCCCCCCCC]

A mixture of L101-5 (100 mg, 0.087 mmol) and 10% Pd (OH) 2/C (35 mg) in EtOAc (2.5 mL) was stirred under a hydrogen balloon for 70 h. It was then filtered through Celite. The Celite was rinsed with EtOAc (5 mL×3). Concentration of the filtrate provided crude material which was purified by flash column chromatography (SiO₂: ethyl acetate/hexane 0-100% with 1% triethylamine in the eluent) to yield Compound CC-100 as a light-yellow oil (78 mg, 56%). ¹H-NMR (300 MHz, CDCl₃) δ 4.49 (t, J=5.5 Hz, 2H), 4.04 (1, J=6.8 Hz, 4H), 3.61-3.32 (m, 10H), 2.92-2.49 (m, 6H), 2.38 (t, J=7.7 Hz, 4H), 2.00-1.43 (m, 16H), 1.42-1.12 (m, 72H), 0.88 (t, J=7.1 Hz, 12H); MS (CI): m/z [M+H]⁺ 1066.9; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.68 min, purity: >99%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=16.3 min, purity: 91.0%.

### Synthesis of CO-2

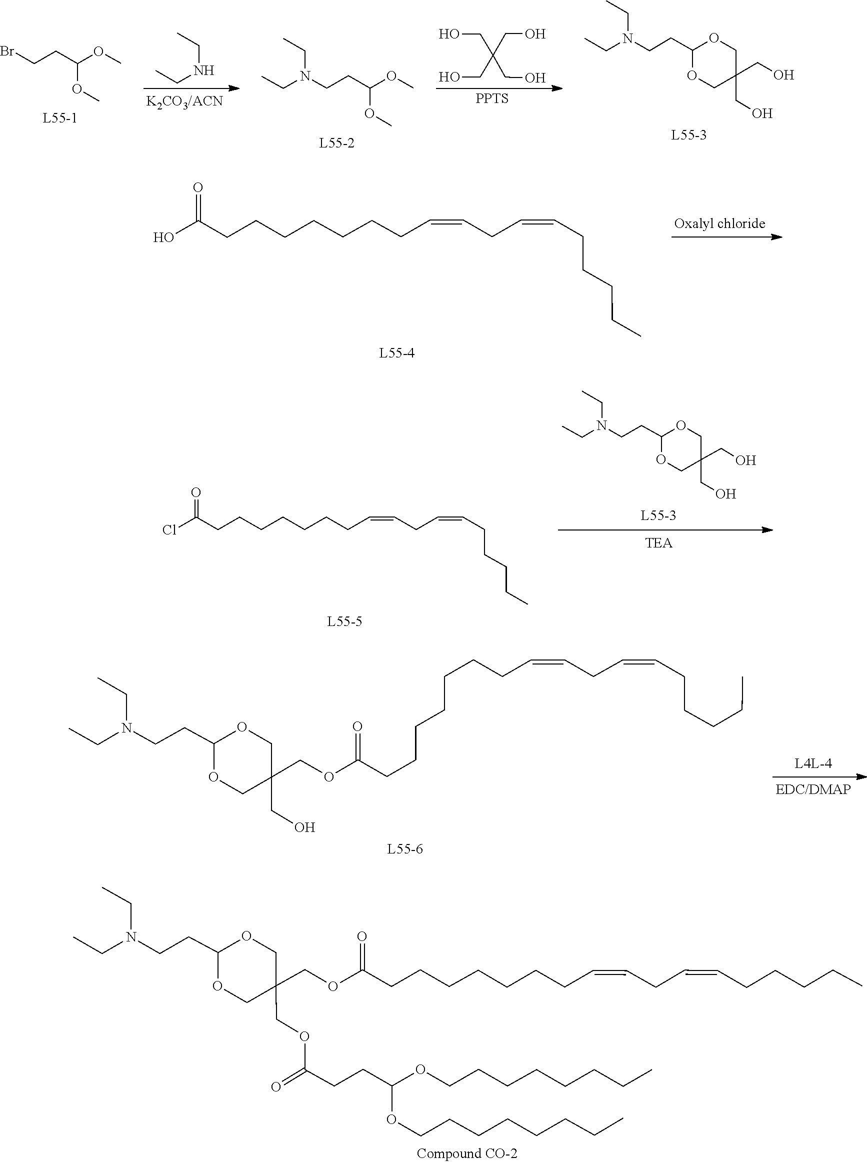

### Synthesis of N, N-diethyl-3,3-dimethoxypropan-1-amine (L55-2)

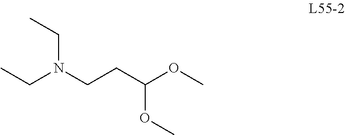

L55-1 (3 g, 16 mmol), diethyl amine (3.39 mL, 32 mmol) and potassium carbonate (4.5 g, 32 mmol) were added to CH₃CN (15 mL) in a pressure vial. The vial was sealed and stirred at 70° C. for 18 hours. The reaction mixture was then diluted with water (200 mL), and extracted by ethyl acetate (200 mL×2). The combined organic fractions were dried over Na₂SO₄ and concentrated to obtain product N, N-diethyl-3,3-dimethoxypropan-1-amine L55-2 as brown oil (2.26 g, 80%); ¹H-NMR (300 MHz, CDCl₃) δ 4.43 (t, 1H), 3.32 (s, 6H), 2.58-2.36 (m, 6H), 1.84-1.65 (m, 2H), 1.01 (t, 6H).

### Synthesis of (2-(2-(diethylamino)ethyl)-1,3-dioxane-5,5-diyl)dimethanol (L55-3)

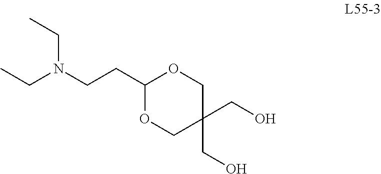 [SMILES: CCN(CC)CCC1OCC(CO)(CO)CO1]

Pentaerythritol (1.482 g, 11 mmol) was dissolved in DMF (20 mL) at 100° C. To this clear solution was added L55-2 (1.9 g, 11 mmol) and Pyridinium p-toluenesulfonate (PPTS) (2.993 g, 12 mmol). To allow MeOH to escape, the reaction was stirred in an open round bottom flask at 100° C. for 20 hours. DMF was evaporated, and the crude product was then dissolved in a small amount of DCM and subjected to silica gel column using 0-40% MeOH in DCM with 2% NH₄OH as eluent to afford L55-3 as yellow oil (2.3 g, 86%). ¹H-NMR (300 MHz, CDCl₃) δ 4.58 (t, 1H), 4.00 (dd, 4H), 3.49 (dd, 4H), 2.68-2.52 (m, 6H), 1.84-1.65 (m, 2H), 1.06 (t, 6H). CIMS m/z [M+H]⁺ 248.3.

### Synthesis of (9Z,12Z)-octadeca-9,12-dienoyl chloride (L55-5)

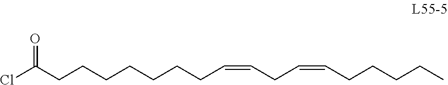 [SMILES: CCCCC/C=C\C/C=C\CCCCCCCC(=O)Cl]

L55-4 (283 mg, 1.01 mmol) was dissolved in DCM (5 mL) at 0° C. After adding DMF (40 μL, 1%, wt %), a solution of oxalyl chloride (128 μL, 1.51 mmol) in DCM (2 ml) was added dropwise through a syringe. After the addition was complete, the reaction mixture was allowed to warm to room temperature and stirred for 2 hours. After evaporation of the solvent, the crude product L55-5 was used for the next reaction step without further purification.

### Synthesis of (2-(2-(diethylamino)ethyl)-5-(hydroxymethyl)-1,3-dioxan-5-yl)methyl (9Z,12Z)-octadeca-9,12-dienoate (L55-6)

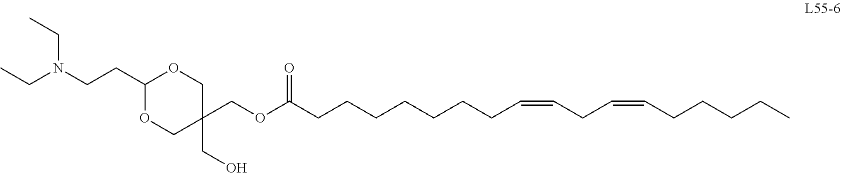 [SMILES: CCCCC/C=C\C/C=C\CCCCCCCC(=O)OCC1(CO)COC(CCN(CC)CC)OC1]

L55-5 (from last step) was dissolved in DMF (2 mL) then added to a stirred solution of L55-3 (300 mg, 1.21 mmol) and triethyl amine (280 μL, 2.01 mmol) in DMF (8 mL). The reaction mixture was stirred for 20 hours at room temperature. When TLC (10% MeOH in DCM with 1% NH₄OH, R_{f}=0.4) showed the completion of reaction, it was then diluted by EA (10 mL) and washed by water (10 mL×2). The organic fraction was evaporated to obtain crude product which was then subjected to silica gel column using 0-15% MeOH in DCM with 1% NH₄OH as eluent to afford L55-6 as light-yellow oil (125 mg, 25%). ¹H-NMR (300 MHz, CDCl₃) δ 5.42-5.24 (m, 4H), 4.51-4.57 (m, 1H), 4.48 (s, 1H), 3.99-3.76 (m, 4H), 3.59-3.44 (m, 2H), 3.19 (s, 1H), 2.76 (dd, 2H), 2.68-2.44 (m, 6H), 2.39-2.24 (m, 2H), 2.10-1.94 (m, 4H), 1.85-1.70 (m, 3H), 1.70-1.46 (m, 5H), 1.40-1.19 (m, 16H), 1.10-0.94 (m, 6H), 0.88 (t, 3H). CIMS m/z [M+H]⁺ 510.4.

### Synthesis of (5-(((4,4-bis(octyloxy) butanoyl)oxy)methyl)-2-(2-(diethylamino)ethyl)-1,3-dioxan-5-yl)methyl (9Z,12Z)-octadeca-9,12-dienoate (Compound CO-2)

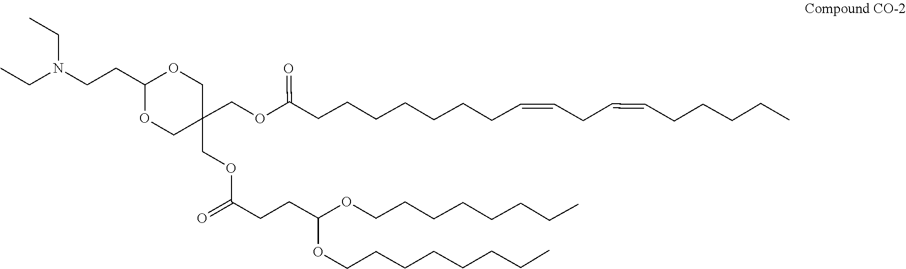 [SMILES: CCCCC/C=C\C/C=C\CCCCCCCC(=O)OCC1(COC(=O)CCC(OCCCCCCCC)OCCCCCCCC)COC(CCN(CC)CC)OC1]

L4L-4 (121 mg, 0.24 mmol) and EDC/DMAP (225 mg, 0.8 mmol/39 mg, 0.22 mmol) were dissolved in DCM (2 mL). After stirring for 5 minutes the solution became clear. L55-6 (150 mg, 0.2 mmol) was then added to the above solution, and the reaction mixture was stirred for 2 hours at room temperature. When TLC (10% MeOH in DCM with 1% NH₄OH, R_{f}=0.6) showed the completion of reaction, the solvent was evaporated. The obtained crude product was then subjected to silica gel column using 0-10% MeOH in DCM with 1% NH₄OH as eluent to afford Compound CO-2 as yellow oil (151 mg, 62%). ¹H-NMR (300 MHz, CDCl₃) δ 5.42-5.24 (m, 4H), 4.58-4.51 (m, 1H), 4.50-4.43 (m, 1H), 4.35 (s, 1H), 4.34 (s, 1H), 3.99-3.89 (m, 2H), 3.82 (s, 2H), 3.68-3.50 (m, 4H), 3.44-3.32 (m, 2H), 2.76 (dd, 2H), 2.57 (bs, 6H), 2.38 (q, 2H), 2.29 (q, 2H), 2.01 (q, 4H), 1.96-1.85 (m, 2H), 1.80 (bs, 2H), 1.67-1.46 (m, 10H), 1.40-1.16 (m, 36H), 1.09-0.95 (m, 6H), 0.92-0.76 (m, 9H); CIMS m/z [M+H]⁺ 837.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=6.51 min, purity: >99.9%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 2θ±2° C., detector: CAD, t_{R}=6.43 min, purity: 98.1%.

### Synthesis of CO-1

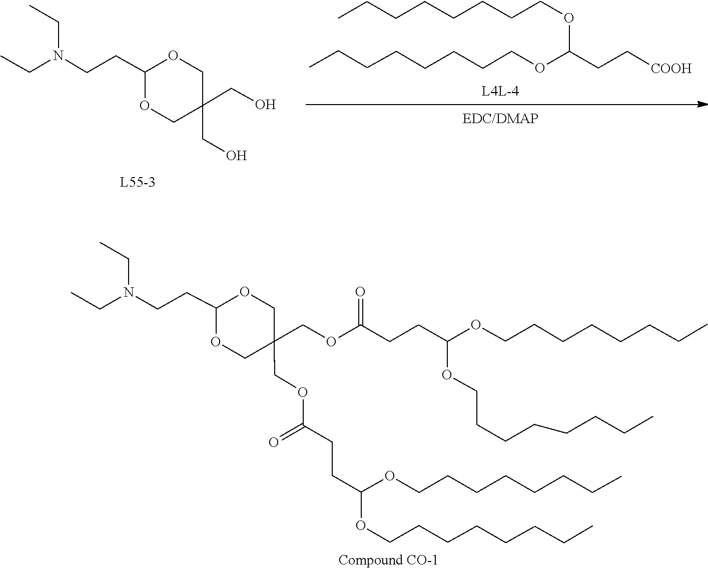 [SMILES: CCN(CC)CCC1OCC(CO)(CO)CO1 ; CCCCCCCCOC(CCC(=O)O)OCCCCCCCC ; CCCCCCCCOC(CCC(=O)OCC1(COC(=O)CCC(OCCCCCCCC)OCCCCCCCC)COC(CCN(CC)CC)OC1)OCCCCCCCC]

### Synthesis of (2-(2-(diethylamino)ethyl)-1,3-dioxane-5,5-diyl)bis(methylene)bis(4,4-bis(octyloxy)butanoate) (Compound CO-1)

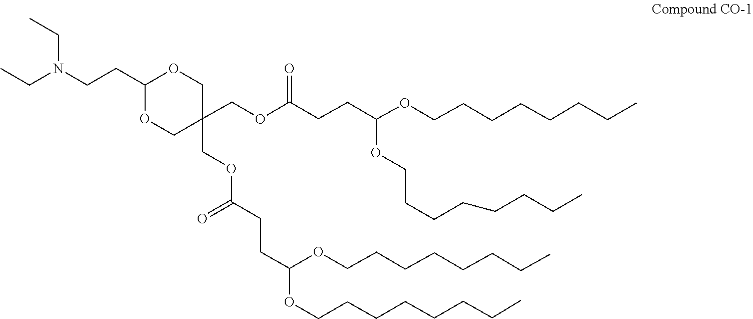 [SMILES: CCCCCCCCOC(CCC(=O)OCC1(COC(=O)CCC(OCCCCCCCC)OCCCCCCCC)COC(CCN(CC)CC)OC1)OCCCCCCCC]

L4L-4 (368 mg, 1.06 mmol) and EDC/DMAP (373 mg, 1.9 mmol/24 mg, 0.19 mmol) were dissolved in DCM (5 mL). After stirring for 5 minutes, the solution became clear. The starting material L55-3 (120 mg, 0.48 mmol) was then added to the above solution. The reaction mixture was stirred for 4 hours at room temperature. When TLC (10% MeOH in DCM with 1% NH₄OH, R_{f}=0.6) showed the completion of reaction, the solvent was evaporated. The obtained crude product was then subjected to silica gel column using 0-20% MeOH in DCM with 1% NH₄OH to afford Compound L56 as yellow oil (240 mg, 56%). ¹H-NMR (300 MHz, CDCl₃) δ 4.53 (t, 1H), 4.51-4.43 (m, 2H), 4.35 (s, 2H), 3.95 (s, 1H), 3.91 (s, 1H), 3.82 (s, 2H), 3.66-3.48 (m, 6H), 3.45-3.34 (m, 4H), 2.61-2.44 (m, 6H), 2.38 (q, 4H), 1.98-1.85 (m, 4H), 1.82-1.70 (m, 2H), 1.66-1.44 (m, 12H), 1.38-1.14 (m, 41H), 1.02 (t, 6H), 0.87 (t, 12H); CIMS m/z [M+H]⁺ 901.7. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=6.57 min, purity: >99.9%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=6.34 min, purity: 99.6%.

### Synthesis of CO-3

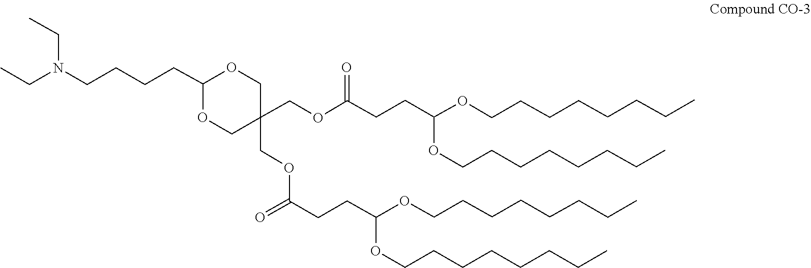 [SMILES: CCCCCCCCOC(CCC(=O)OCC1(COC(=O)CCC(OCCCCCCCC)OCCCCCCCC)COC(CCCCN(CC)CC)OC1)OCCCCCCCC]

Compound CO-3 was made by an analogous method as described for Compound CO-1, starting with (2-(4-(diethylamino)butyl)-1,3-dioxane-5,5-diyl)dimethanol in place of L55-3. (2-(4-(diethylamino)butyl)-1,3-dioxane-5,5-diyl)dimethanol was made by an analogous method as described for L55-3, starting with 5-bromo-1,1-dimethoxypentane in place of L55-1. Compound CO-3 was obtained as colorless oil (0.55 g, 44%); ¹H-NMR (300 MHz, CDCl₃) δ 4.52-4.43 (m, 3H), 4.34 (s, 2H), 4.15 (s, 1H), 3.94-3.90 (m, 2H), 3.81 (s, 2H), 3.59-3.52 (m, 5H), 3.42-3.36 (m, 4H), 2.51-2.46 (m, 3H), 2.40-2.35 (m, 6H), 1.98-1.88 (m, 5H), 1.66-1.49 (m, 11H), 1.44-1.08 (m, 43H), 0.99 (t, 6H), 0.86 (t, 12H); CIMS m/z 928.7 [M+H]+. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=7.80 min, purity: >99%; UPLC column: Waters Aquity UPLC® CSH™, C18, 1.7 μm, 3.0×150 mmol, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=14.34 min, purity: 93.4%.

### Synthesis of AC-1

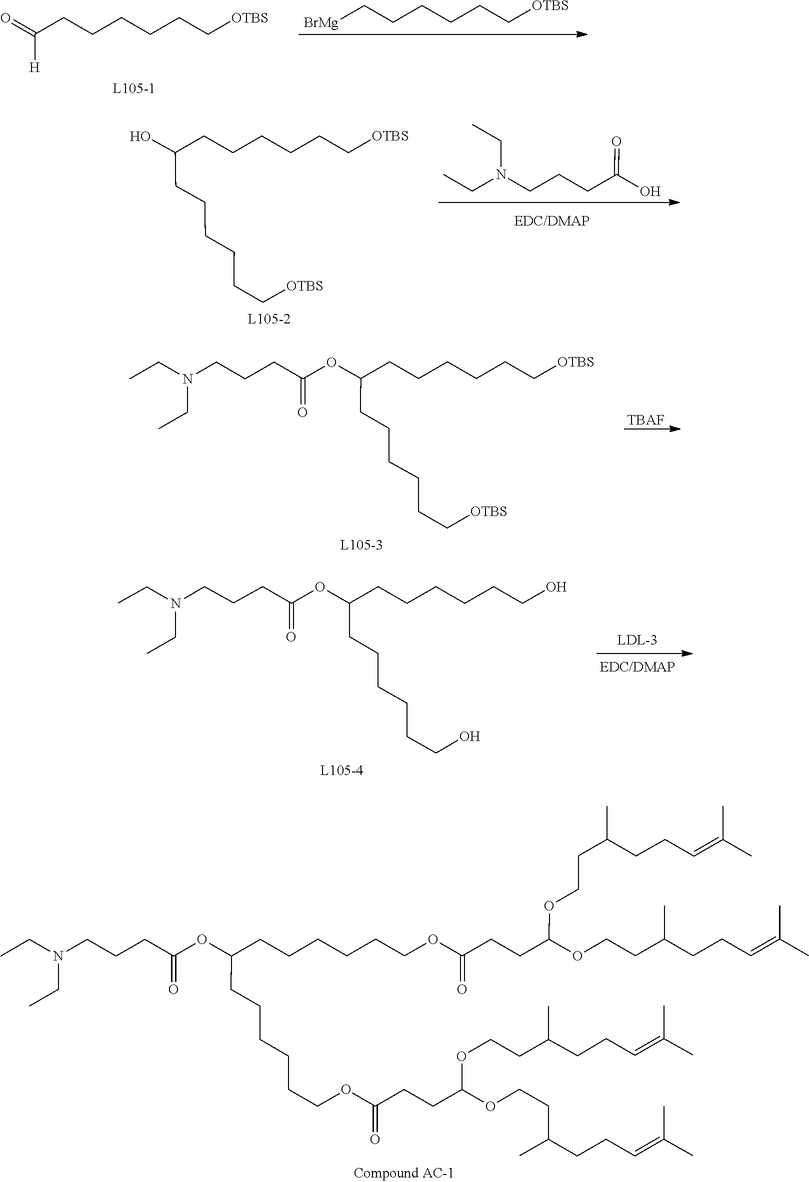

### Synthesis of 2,2,3,3,19,19,20,20-octamethyl-4,18-dioxa-3,19-disilahenicosan-11-ol (L105-2)

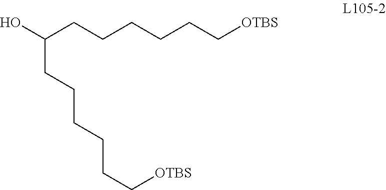 [SMILES: CC(C)(C)[Si](C)(C)OCCCCCCC(O)CCCCCCO[Si](C)(C)C(C)(C)C]

A solution of L105-1 (600 mg, 2.45 mmol) in THF (7 mL) was added to Grignard reagent (2.35 g, 7.35 mmol) under nitrogen atmosphere and the reaction mixture was stirred at room temperature for 2 hours. When TLC (20% EtOAc in hexane, R_{f}=0.8) showed the completion of reaction, it was then filtered and extracted by EtOAc/H₂O. The organic fraction was evaporated to obtain crude product which was then subjected to silica gel column using 0-20% EtOAc in hexane as eluent to afford L105-2 as light-yellow oil (780 mg, 69%). ¹HNMR (300 MHz, CDCl₃) δ 3.59 (t, 3H), 1.57-1.20 (m, 20H), 0.90 (s, 18H), 0.04 (s, 12H). CIMS m/z [M+H]⁺ 261.4.

### Synthesis of 2,2,3,3,19,19,20,20-octamethyl-4,18-dioxa-3,19-disilahenicosan-11-yl 4-(diethylamino)butanoate (L105-3)

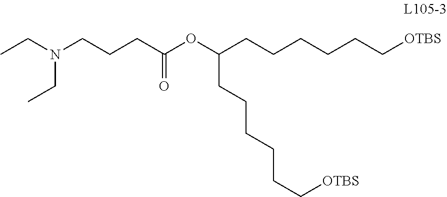 [SMILES: CCN(CC)CCCC(=O)OC(CCCCCCO[Si](C)(C)C(C)(C)C)CCCCCCO[Si](C)(C)C(C)(C)C]

4-(diethylamino)butanoic acid (458 mg, 2.88 mmol) and EDC/DMAP (1.2 g, 7.67 mmol/210 mg, 2.11 mmol) were dissolved in DCM/DIPEA (7/0.408 mL). After stirring for 5 minutes the solution turned clear. The starting material L105-2 (720 mg, 1.92 mmol) was then added to the above solution, the reaction mixture was stirred for 20 hours at room temperature. When TLC (10% MeOH in DCM with 1% NH₄OH, R_{f}=0.6) showed the completion of reaction, the solvent was evaporated. The obtained crude product was then subjected to silica gel column using 0-10% MeOH in DCM with 1% NH₄OH as eluent to afford L105-3 as yellow oil (800 mg, 85%). ¹HNMR (300 MHz, CDCl₃) δ 4.91-4.79 (m, 1H), 3.58 (t, 4H), 2.58-2.38 (m, 6H), 2.30 (t, 2H), 1.71-1.69 (m, 2H), 1.54-1.40 (m, 8H), 1.38-1.13 (m, 12H), 1.01 (t, 6H), 0.89 (s, 18H), 0.04 (s, 12H). CIMS m/z [M+H]⁺ 602.0.

### Synthesis of 1,13-dihydroxytridecan-7-yl 4-(diethylamino)butanoate (L105-4)

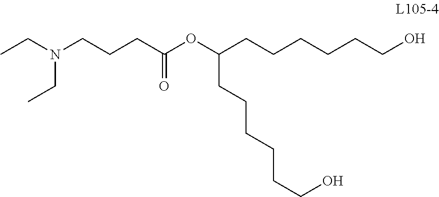 [SMILES: CCN(CC)CCCC(=O)OC(CCCCCCO)CCCCCCO]

The starting material L105-3 (800 mg, 1.33 mmol) was dissolved in THF (5 mL) followed by adding 1.0 M TBAF solution (in THF, 2.5 mL, 1.99 mmol). The reaction mixture was then stirred at room temperature for 6 hours. When TLC (10% MeOH in DCM with 1% NH₄OH, R_{f}=0.6) showed the completion of reaction, the solvent was evaporated and the crude product was purified by column chromatography (0-15% MeOH in DCM with 1% NH₄OH) to yield L105-4 as colorless oil (470 mg, 95%). ¹HNMR (300 MHz, CDCl₃) δ 4.91-4.79 (m, 1H), 3.62 (t, 4H), 2.63-2.39 (m, 6H), 2.31 (t, 2H), 1.88-1.68 (m, 2H), 1.62-1.41 (m, 8H), 1.40-1.19 (m, 12H), 1.05 (t, 6H). CIMS m/z [M+H]⁺ 374.3.

### Synthesis of 7-((4-(diethylamino)butanoyl)oxy)tridecane-1,13-diyl bis(4,4-bis((3,7-dimethyloct-6-en-1-yl)oxy)butanoate) (Compound AC-1)

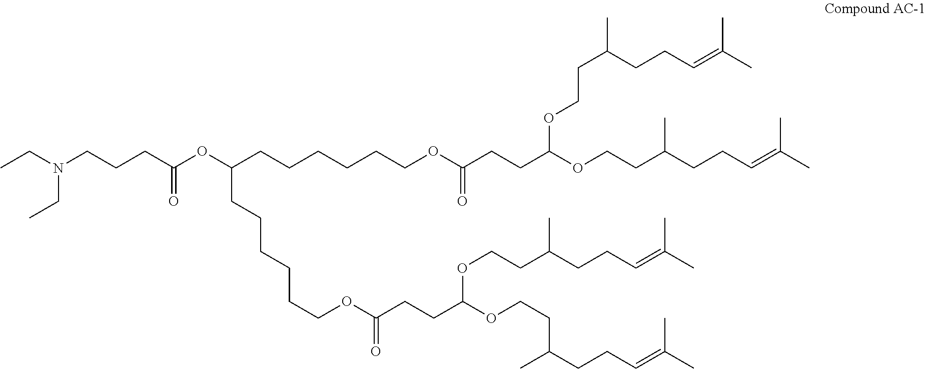 [SMILES: CCN(CC)CCCC(=O)OC(CCCCCCOC(=O)CCC(OCCC(C)CCC=C(C)C)OCCC(C)CCC=C(C)C)CCCCCCOC(=O)CCC(OCCC(C)CCC=C(C)C)OCCC(C)CCC=C(C)C]

L4L-3 (1.1 g, 2.77 mmol) and EDC/DMAP (1.94 g, 10.09 mmol/338 mg, 2.77 mmol) were dissolved in DCM (30 mL). After stirring for 5 minutes the solution turned clear. L105-4 (470 mg, 1.26 mmol) was then added to the above solution, and the reaction mixture was stirred for 20 hours at room temperature. When TLC (10% MeOH in DCM with 1% NH₄OH, R_{f}=0.6) showed the completion of reaction, the solvent was evaporated. The obtained crude product was then subjected to silica gel column using 0-10% MeOH in DCM with 1% NH₄OH as eluent to afford Compound AC-1 as light-yellow oil (1.1 g, 81%). ¹HNMR (300 MHz, CDCl₃) δ 5.15-5.01 (m, 4H), 4.83 (quint, 1H), 4.48 (t, 2H), 4.04 (1, 4H), 3.68-3.54 (m, 4H), 3.49-3.36 (m, 4H), 2.70-2.42 (m, 4H), 2.42-2.26 (m, 6H), 2.08-1.86 (m, 12H), 1.86-1.72 (m, 2H), 1.71-1.42 (m, 40H), 1.42-1.24 (m, 20H), 1.22-0.98 (m, 12H), 0.88 (d, 12H). CIMS m/z [M+H]⁺ 1130.9. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 70% to 100% in 5 min, then 100% for 10 min. Flow rate: 1 mL/min, column temperature: 20±2° C., detector: ELSD, t_{R}=6.93 min, purity: >99%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 70% to 100% in 5 min, then 100% for 15 min. Flow rate: 1 mL/min, column temperature: 20±2° C., detector: CAD, t_{R}=7.11 min, purity: 95.1%.

### Synthesis of AC-2

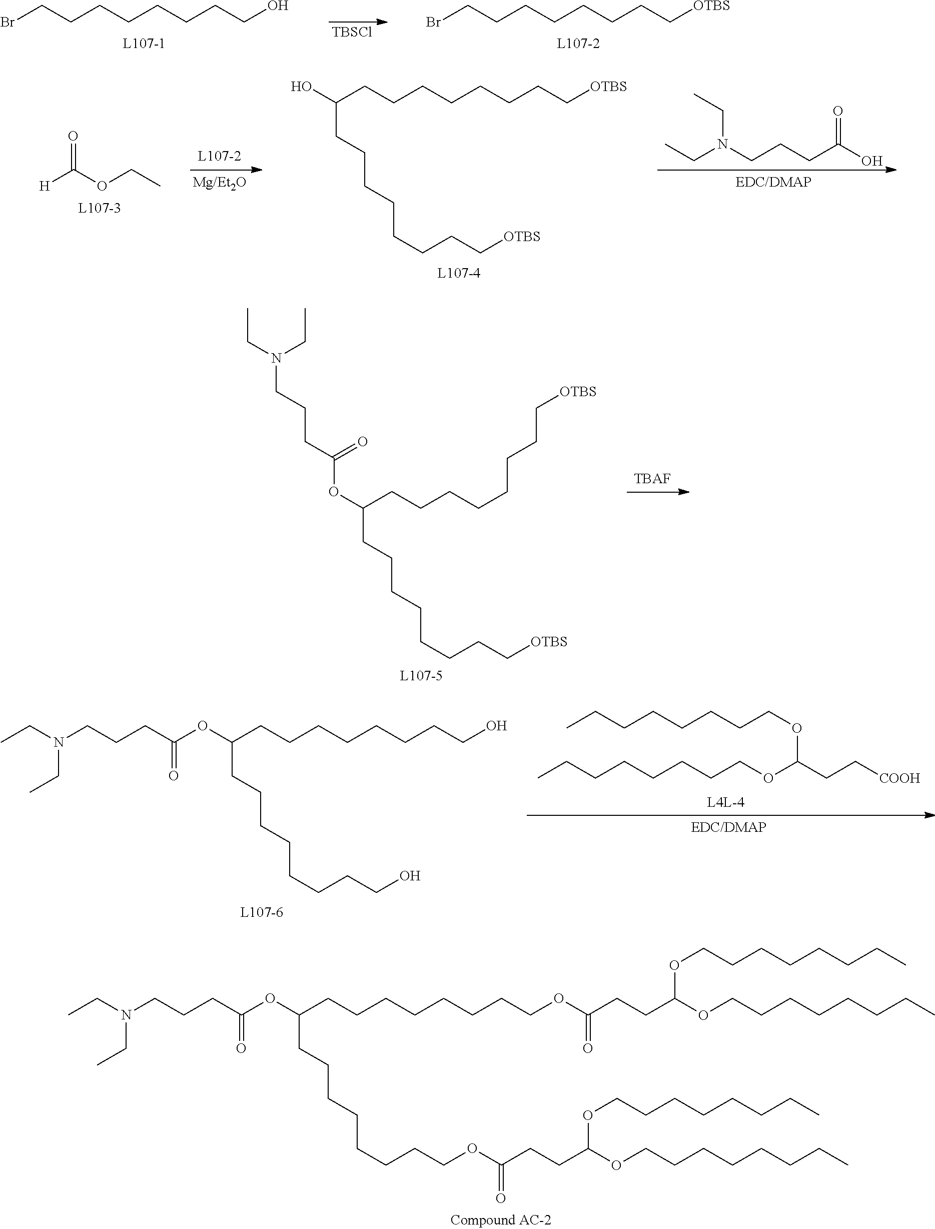

### Synthesis of ((8-bromooctyl)oxy) (tert-butyl)dimethylsilane (L107-2)

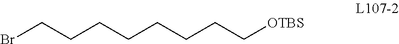 [SMILES: CC(C)(C)[Si](C)(C)OCCCCCCCCBr]

To a 500 mL round bottom flask L107-1 (25.0 g, 119.54 mmol) and TBSCI (36.04 g, 239.09 mmol) were taken. To this was added anhydrous dichloromethane (70 mL) and the reaction mixture was stirred for 15 min at room temperature. Imidazole (32.55 g, 487.17 mmol) was added, and the reaction was stirred at room temperature for 18 h. After completion of the reaction the solvent was removed under vacuum. Residual paste was partitioned between ethyl acetate and water. The aqueous phase was extracted with ethyl acetate (100 mL×3). The combined ethyl acetate fraction was washed with water (100 mL) twice and with brine (100 mL) once. The ethyl acetate layer was separated and dried over anhydrous sodium sulphate. The ethyl acetate was removed under vacuum to give crude product. This crude was loaded on to a 330 g flash silica column and was purified by flash chromatography (SiO₂: ethyl acetate/hexane 0-50%) to yield L107-2 (29.5 g, 76%) as colorless oil. ¹HNMR (CDCl₃) δ 3.59 (t, J=8 Hz, 2H), 3.39 (t, J=8 Hz, 2H), 1.90-1.84 (m, 2H), 1.62-1.37 (m, 4H), 1.35-1.25 (m, with sharp singlet at 1.30, 6H), 0.89 (s, 9H), 0.04 (s, 6H); MS (CI): m/z [M+]⁺ 324.41.

### Synthesis of 2,2,3,3,23,23,24,24-octamethyl-4,22-dioxa-3,23-disilapentacosan-13-ol (L107-4)

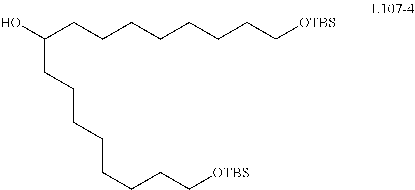 [SMILES: CC(C)(C)[Si](C)(C)OCCCCCCCCC(O)CCCCCCCCO[Si](C)(C)C(C)(C)C]

To a dry 1 L three-neck round bottom flask equipped with magnetic stir bar and fitted with a reflux condenser and rubber septa was added magnesium turnings (4.84 g, 202.48 mmol) and one tiny crystal of iodine under inert conditions, followed by addition of anhydrous THF (50 mL). The reaction was allowed to stir for 30 min, until the iodine color faded. To this reaction was added 1,2-dibromoethane (0.6 mL) and stirring was continued for 60 min. L107-2 (8.72 g, 26.97 mmol) was dissolved in anhydrous THF (50 mL) and added dropwise to the reaction. After completion of the addition, the reaction was refluxed for 2 h then allowed to cool to room temperature, followed by cooling in ice-water bath for 30 min. To this reaction was added L107-3 (600 mg, 8.09 mmol) and the reaction was stirred at room temperature for 18 h. The reaction was quenched using 300 mL saturated ammonium chloride solution followed by partitioning in ethyl acetate: water. Brine (200 mL) was added and the mixture was filtered through celite to break the emulsion formation. The product was extracted three times with ethyl acetate. The combined ethyl acetate layers were dried over anhydrous sodium sulphate. The ethyl acetate was removed under vacuum to give crude product. This crude was loaded on to 220 g flash silica column and was purified by flash chromatography (SiO₂: ethyl acetate/hexane 0-50%) to yield L107-4 (4.18 g, 87%) as colorless oil. ¹HNMR (300 MHz, CDCl₃) δ: 3.58 (t, J=8.0 Hz, 5H), 1.57-1.25 (m, with sharp singlet at 1.29, 29H), 0.89 (s, 18H), 0.04 (s, 12H); CIMS m/z [M+H]⁺ 518.0.

### Synthesis of 2,2,3,3,23,23,24,24-octamethyl-4,22-dioxa-3,23-disilapentacosan-13-yl (diethylamino)butanoate (L107-5)

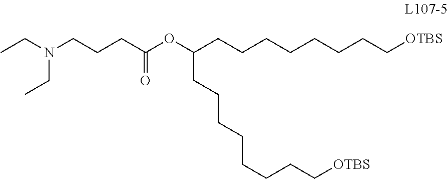 [SMILES: CCN(CC)CCCC(=O)OC(CCCCCCCCO[Si](C)(C)C(C)(C)C)CCCCCCCCO[Si](C)(C)C(C)(C)C]

To a 250 mL round bottom flask was added diethylamino butyric acid (2.63 g, 13.4 mmol), EDC (5.45 g, 28.43 mmol) and DMAP (1.98 g, 16.25 mmol) in anhydrous dichloromethane (30 mL), and the reaction was stirred for 15 min. To this was added N, N-Diisopropylethylamine (6.01 mL, 34.53 mmol) along with L107-4 (2.1 g, 4.06 mmol) in anhydrous dichloromethane (10 mL) and the reaction mixture was stirred under nitrogen at room temperature for 48 h. After completion of reaction, about 20 g of silica gel (neutralized with triethylamine) was added and the contents were stirred well to yield a uniform mixture. Solvent was removed from this mixture under vacuum. The residue was loaded on to an empty column cartridge, which was then attached to flash purification system loaded with 40 g flash silica column (equilibrated with 1% triethylamine in hexane) and purified by flash chromatography (SiO₂: ethyl acetate/hexane (with 1% triethylamine) 0-10%) to yield L107-5 (2.67 g, 96%) as a clear oil. ¹HNMR (300 MHz, CDCl₃) δ: 4.85 (t, J=7.0 Hz, 1H), 3.58 (t, J=7.0 Hz, 4H), 2.61-2.40 (m, 8H), 1.81-1.71 (m, 2H), 1.62-1.25 (m, with sharp singlet at 1.26, 28H), 0.98 (t, J=7.0 Hz, 6H), 0.89 (s, 18H), 0.04 (s, 12H); CIMS m/z [M+H]⁺ 659.29.

### Synthesis of 1,17-dihydroxyheptadecan-9-yl 4-(diethylamino)butanoate (Compound L107-6)

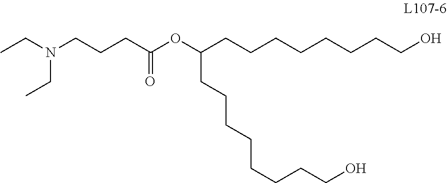 [SMILES: CCN(CC)CCCC(=O)OC(CCCCCCCCO)CCCCCCCCO]

To a 100 mL round bottom flask was added L107-5 (2.5 g, 1.0 eq) and TBAF (3.67 g, 3.7 eq). To this flask was added anhydrous THF (10 mL) and the reaction mixture was stirred at room temperature under inert conditions for 24 h. After completion of the reaction the solvent was removed under vacuum. Residue paste was loaded on to a prepacked column cartridge, which was then attached to flash purification system loaded with 40 g flash silica column and was purified by flash chromatography (0-10% Dichloromethane:Methanol) to yield L107-6 (1.34 g, 82%) as a clear oil. ¹H-NMR (300 MHz, CDCl₃) δ: 4.87 (t, J=7.0 Hz, 1H), 3.62 (t, J=7.0 Hz, 4H), 3.41-3.29 (m, 6H), 2.61-2.40 (bs, 2H), 1.94 (t, J=7.0 Hz, 2H), 1.82-1.25 (m, with sharp singlet at 1.27, 24H), 0.98-0.96 (m, 6H), 0.92 (t, J=7.0 Hz, 6H); CIMS m/z [M+H]⁺ 430.6.

### Synthesis of 9-((4-(diethylamino)butanoyl)oxy) heptadecane-1,17-diyl bis(4,4-bis(octyloxy) butanoate) (Compound AC-2)

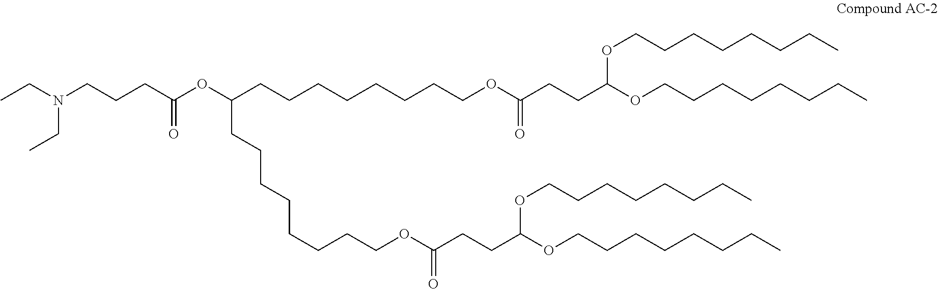 [SMILES: CCCCCCCCOC(CCC(=O)OCCCCCCCCC(CCCCCCCCOC(=O)CCC(OCCCCCCCC)OCCCCCCCC)OC(=O)CCCN(CC)CC)OCCCCCCCC]

To a 250 mL round bottom flask was added L4L-4 (2.24 g, 6.51 mmol), EDC (3.12 g, 16.29 mmol) and DMAP (995 mg, 8.15 mmol) in anhydrous dichloromethane (30 mL) and the reaction was stirred for 15 min. To this was added L107-6 (700 mg, 1.63 mmol) in anhydrous dichloromethane (10 mL) and the reaction mixture was stirred under nitrogen at room temperature for 48 h. After completion of reaction about 20 g of flash silica (neutralized with triethylamine) was added and the contents were stirred well to get a uniform mixture. Solvent was removed from this mixture under vacuum. The residue was loaded on to an empty flash cartridge, which was then attached to flash purification system loaded with 40 g flash silica column (equilibrated with 1% triethylamine in hexane) and was purified by flash chromatography (SiO₂: ethyl acetate/hexane (with 5% triethylamine) 0-10%) to yield Compound AC-2 (1.70 g, 96%) as a clear oil. ¹HNMR (300 MHz, CDCl₃) δ: 4.87 (t, J=7.0 Hz, 1H), 4.48 (t, J=7.0 Hz, 2H), 4.04 (t, J=7.0 Hz, 4H), 3.58-3.53 (m, 4H), 3.43-3.33 (m, 4H), 2.60-2.30 (m, 10H), 2.00-1.91 (m, 3H), 1.81 (t, J=7.0 Hz, 1H), 1.79-1.50 (m, 18H), 1.45-1.24 (m, with sharp singlet at 1.26, 62H), 1.02 (t, J=7.0 Hz, 6H), 0.87 (t, J=7.0 Hz, 12H); CIMS m/z [M+H]⁺ 1083.74 Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.4 min, purity: >99%; UPLC column: Waters Aquity UPLC® CSH™, C18, 1.7 μm, 3.0×150 mmol, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=16.6 min, purity: 93.85%.

### Synthesis of AT-1

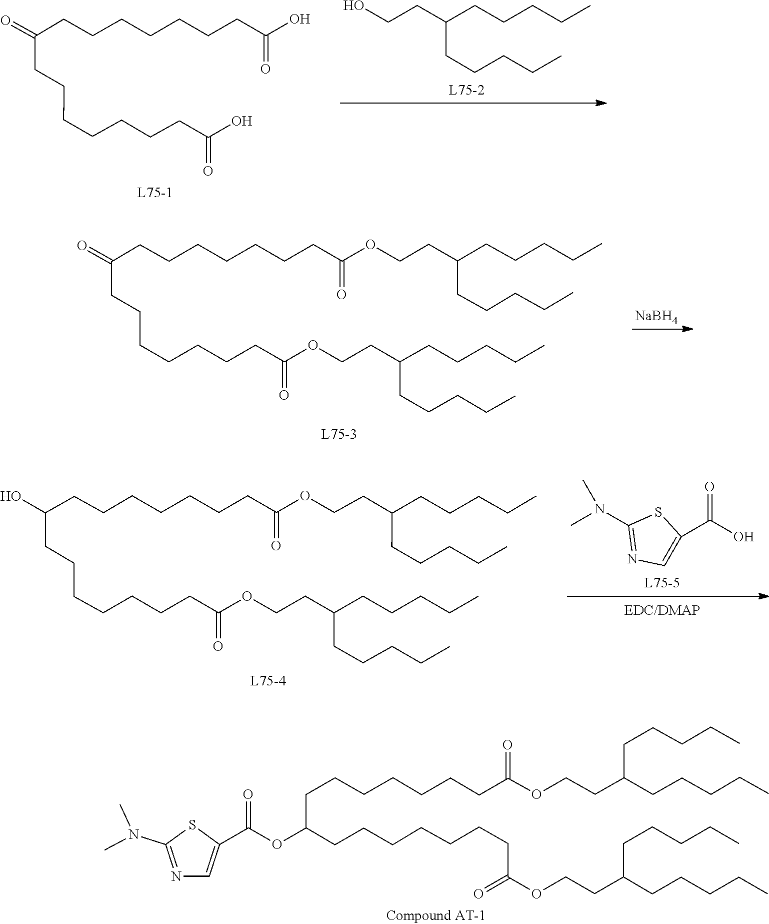

### Synthesis of bis(3-pentyloctyl) 9-oxoheptadecanedioate (L75-3)

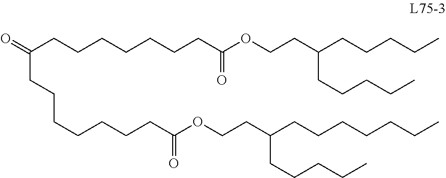 [SMILES: CCCCCCCC(CCCCC)CCOC(=O)CCCCCCCC(=O)CCCCCCCC(=O)OCCC(CCCCC)CCCCC]

To a solution of L75-1 (1.8 g, 5.99 mmol) and L75-2 (2.52 g, 12.58 mmol) in DCM (20 mL) at room temperature was added DMAP (70 mg) and the reaction was stirred for 10 min, followed by addition of EDC (2.45 g, 12.76 mmol). The reaction was stirred for 48 h at RT under nitrogen atmosphere. The reaction mixture was diluted with DCM (100 mL) and washed with saturated NaHCO₃ solution (2×25 mL), water (25 mL), and brine (25 mL). Organic layer dried over anhydrous Na₂SO₄. Filtration and concentration yielded crude product which was purified by flash column chromatography (SiO₂: 5 to 8% ethyl acetate in hexane gradient) to yield L75-3 as colorless oil (2.1 g, 54%). ¹H-NMR (300 MHz, CDCl₃) δ 4.09-4.04 (t, 4H), 2.39-2.34 (t, 4H), 2.29-2.24 (t, 4H), 1.69-1.52 (m, 12H), 1.49-1.25 (m, 46H), 0.89-0.85 (1, 12H). CIMS m/z [M+H]⁺ 680.6.

### Synthesis of bis(3-pentyloctyl) 9-hydroxyheptadecanedioate (L75-4)

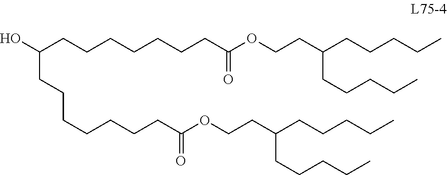 [SMILES: CCCCCC(CCCCC)CCOC(=O)CCCCCCCC(O)CCCCCCCC(=O)OCCC(CCCCC)CCCCC]

To a solution of L75-3 (2.1 g, 3 mmol) in THF (12 mL) and methanol (6 mL) was added sodium borohydride (105 mg, 3.6 mmol) at room temperature. The reaction mixture was stirred under nitrogen atmosphere until TLC (20% EA in hexane, R_{f}=0.5) showed the completion of reaction. The reaction was quenched with HCl (1 M), and all the volatile components were evaporated. The residue was dissolved in diethyl ether and washed with H₂O and brine. The organic phase was dried over Na₂SO₄ and evaporated to obtain crude material which was purified by silica gel column using 0-50% EA in hexane as eluent to yield L75-4 (1.5 g, 70%). ¹H-NMR (300 MHz, CDCl₃) δ 4.07 (t, J=7.2 Hz, 4H), 3.65-3.59 (m, 1H), 3.28 (t, J=7.6 Hz, 4H), 1.58 (m, 10H), 1.53-1.37 (m, 8H), 1.36-1.15 (m, 46H), 0.88 (t, J=7.1 Hz, 12H).

### Synthesis of bis(3-pentyloctyl) 9-((2-(dimethylamino)thiazole-5-carbonyl)oxy) heptadecanedioate (Compound AT-1)

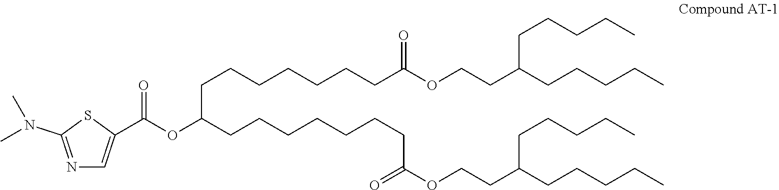 [SMILES: CCCCCC(CCCCC)CCOC(=O)CCCCCCCC(CCCCCCCC(=O)OCCC(CCCCC)CCCCC)OC(=O)c1cnc(N(C)C)s1]

To a solution of L75-4 (500 mg, 0.73 mmol) in DCM (10 mL) was added thiazole carboxylic acid L75-5 (140 mg, 0.8 mmol) and DIPEA (0.4 mL), followed by DMAP (18 mg, 0.15 mmol) and EDC (280 mg, 1.46 mmol). The resulting mixture was stirred at room temperature under nitrogen atmosphere for 18 h. The reaction mixture was diluted with DCM (15 mL) and washed with brine (10 mL). The organic phase was dried over anhydrous Na₂SO₄. Filtration and concentration provided crude material which was purified by flash column chromatography (SiO₂: ethyl acetate/hexane 0-100%) to yield Compound AT-1 as colorless oil (245 mg, 40%); ¹H-NMR (300 MHz, CDCl₃) δ 7.86 (s, 1H), 4.99 (m, 1H), 4.06 (t, J=7.0 Hz, 4H), 3.16 (s, 6H), 2.26 (t, J=7.4 Hz, 4H), 1.70-1.50 (m, 12H), 1.49-1.15 (m, 50H), 0.88 (t, J=7.1 Hz, 12H); CIMS m/z [M+H]⁺ 835; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=9.6 min, purity: >99%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=14.3 min, purity: >99%.

### Synthesis of AT-2

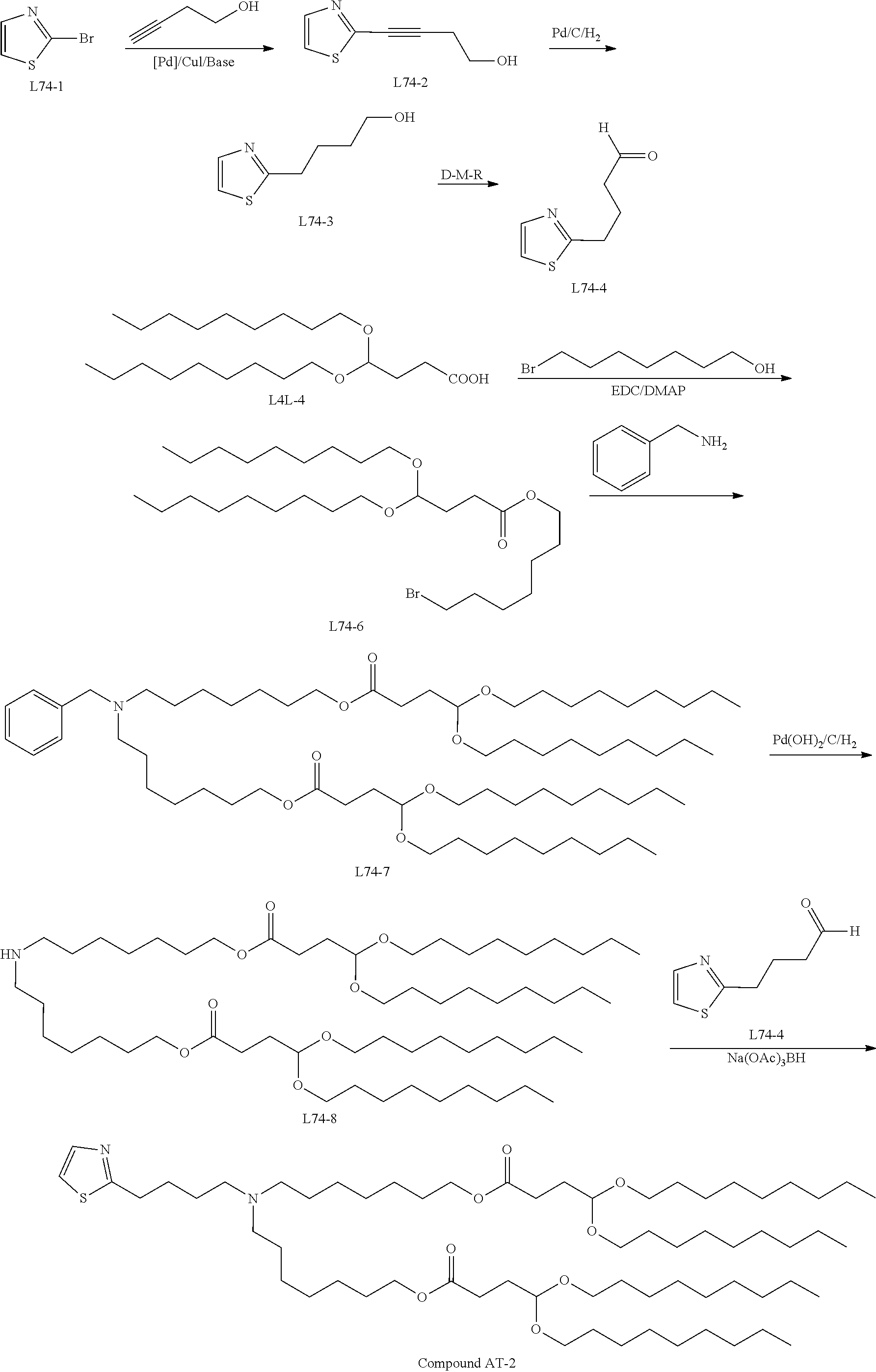

### Synthesis of 4-(thiazol-2-yl) but-3-yn-1-ol (L74-2)

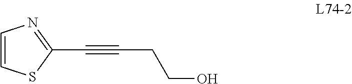

2-Bromothiazole (L74-1, 2 g, 12.18 mmol) was dissolved under nitrogen atmosphere in triethylamine (50 mL). CuI (70 mg, 0.36 mmol, 0.03 eq), Pd(PPh₃)₄ (281.5 mg, 0.24 mmol) and but-3-yn-1-ol (1.28 g, 18.2 mmol) were added and the solution was heated to reflux for 18 h. The solvent was removed under reduced pressure and the residue was purified by flash column chromatography (SiO₂: Hexane/EtOAc 0-100%) to get L74-2 (1.44 g, 77%) as orange oil. ¹H-NMR (300 MHz, CDCl₃) δ 7.76 (d, 1H), 7.28 (d, 1H), 3.87 (t, 2H), 2.76 (t, 2H); APCI-MS: m/z [M+H]⁺ 154.

### Synthesis of 4-(thiazol-2-yl) butan-1-ol (L74-3)

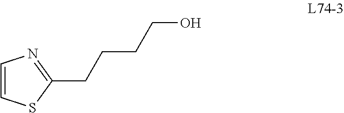

To a solution of L74-2 (1.44 g, 9.35 mmol) in methanol (10 mL) was added 10% Pd/C (100 mg) and 1M HCl in 1,4-dioxane (1 mL) at room temperature. The mixture was subjected to parr-shaker hydrogenator under hydrogen atmosphere for 18 h. The reaction mixture was filtered through a celite pad, concentrated under reduced pressure and the crude was purified by column chromatography (SiO₂: DCM/DCM:MeOH (9:1)) to get L74-3 (1.22 g, 76%) as orange oil. ¹H-NMR (300 MHz, CDCl₃) δ 7.66 (d, 1H), 7.19 (d, 1H), 3.68-3.64 (m, 2H), 3.09-3.04 (m, 2H), 1.90-1.81 (m, 2H), 1.69-1.66 (m, 2H); APCI-MS: m/z [M+H]⁺ 158.

### Synthesis of 4-(thiazol-2-yl) butanal (L74-4)

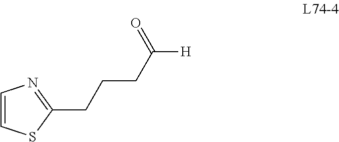

To an ice bath cooled solution of L74-3 (0.61 g, 3.88 mmol) in anhydrous DCM (15 mL) under nitrogen was added Dess-Martin periodinane (1.97 g, 4.65 mmol) in portions. The reaction mixture was warmed to room temperature and stirred for 2 h. The reaction was quenched by slow addition of saturated Na₂SO₃ (2 mL) and NaHCO₃ (2 mL) and stirred for a few minutes, then acetic acid (1 mL) was added. The reaction mixture was concentrated under reduced pressure to dryness and the crude was purified by column chromatography (SiO₂: DCM/DCM:MeOH (9:1)) to get L74-4 (0.3 g, 50%) as light yellow oil. ¹H-NMR (300 MHz, CDCl₃) δ 9.76 (s, 1H), 7.66 (d, 1H), 7.19 (d, 1H), 3.06 (t, 2H), 2.56 (t, 2H), 2.15 (t, 2H); APCI-MS: m/z [M+H]⁺ 156.

### Synthesis of 6-bromohexyl 4,4-bis((3,7-dimethyloctyl)oxy)butanoate (L74-6)

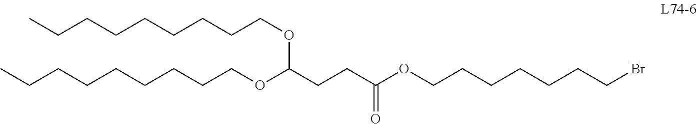 [SMILES: CCCCCCCCCOC(CCC(=O)OCCCCCCCBr)OCCCCCCCCC]

To an oven dried 100 mL round bottom flask containing a solution of acid L74-5 (1.0 g, 2.69 mmol) in dichloromethane (20 mL) under nitrogen was added EDC (1.08 g, 5.38 mmol), DMAP (163 mg, 1.35 mmol). After completion of the addition, the mixture was stirred at room temperature for 15 min. L4L-4 (0.63 g, 3.22 mmol) was then added, and the reaction mixture was stirred at room temperature for 20 h. The reaction mixture was diluted with dichloromethane and water and the resulting phases were separated. The aqueous phase was extracted with EtOAc (50 mL). Combined organic extracts were washed with H₂O (60 mL×2) and dried over anhydrous MgSO₄. Filtration and concentration provided crude product which was purified by flash column chromatography (SiO₂: 0 to 10% ethyl acetate in hexanes gradient) to yield L74-6 as colorless oil (960 mg, 65%); ¹H-NMR (300 MHz, CDCl₃) δ: 4.48 (t, J=5.49 Hz, 1H), 4.05 (t, J=6.6 Hz, 2H), 3.56 (m, 2H), 3.4 (m, 4H), 2.37 (t, J=7.41 Hz, 2H), 1.82-1.95 (m, 4H), 1.52-1.62 (m, 6H), 1.43 (m, 2H), 1.25-1.35 (m, 28H), 0.87 (m, 6H).

### Synthesis of (benzylazanediyl)bis(heptane-7,1-diyl)bis(4,4-bis(nonyloxy)butanoate) (L74-7)

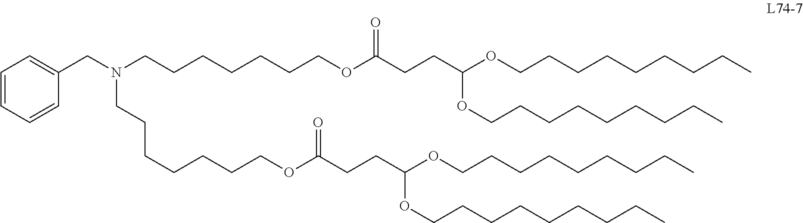 [SMILES: CCCCCCCCCOC(CCC(=O)OCCCCCCCN(CCCCCCCOC(=O)CCC(OCCCCCCCCC)OCCCCCCCCC)Cc1ccccc1)OCCCCCCCCC]

To a 100 mL round bottom flask containing L74-6 (1.69 g, 3.08 mmol), benzyl amine (1.28 mg, 1.23 mmol), K₂CO₃ (1.27 g, 9.24 mmol), and KI (100 mg, 0.60 mmol) was added anhydrous acetonitrile (10 mL) along with cyclopentyl methyl ether (CPME) (10 mL) and the reaction mixture was stirred under reflux at 90° C. for 48 h. After completion of the reaction the solvent was removed under vacuum and purified by flash chromatography (SiO₂: hexane (1% TEA)/ethyl acetate (0-100%)) to get L74-7 (580 mg, 45%). ¹H-NMR (300 MHz, CDCl₃) δ 7.30-7.25 (m, 5H), 4.48 (dd, 2H), 4.05 (t, 4H), 3.57-3.40 (m, 8H), 2.39-2.37 (m, 8H), 1.87-1.95 (m, 6H), 1.58-1.52 (m, 24H), 1.45-1.05 (m, 56H), 0.89-0.87 (m, 12H); APCI-MS: m/z [M+H]⁺ 1045.

### Synthesis of azanediylbis(heptane-7,1-diyl)bis(4,4-bis(nonyloxy)butanoate) (L74-8)

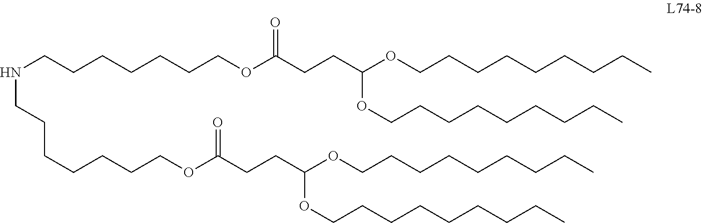 [SMILES: CCCCCCCCCOC(CCC(=O)OCCCCCCCNCCCCCCCOC(=O)CCC(OCCCCCCCCC)OCCCCCCCCC)OCCCCCCCCC]

A mixture of L74-7 (395 mg, 0.37 mmol) and 20% Pd (OH) 2/C (132 mg) in ethyl acetate (10 mL) was hydrogenated at room temperature until all starting material was consumed. The reaction mixture was diluted with ethyl acetate (100 mL) and then filtered through Celite, washed with ethyl acetate and methanol. The solvent was removed under vacuum to dryness and the crude was purified via flash column chromatography (SiO₂: hexane (10% triethyl amine)/ethyl acetate 0-38%) to get L74-8 (310 mg, 86%) as light-yellow oil. ¹H-NMR (300 MHz, CDCl₃) δ 4.48 (t, 2H), 4.04 (t, 4H), 3.57-3.38 (m, 8H), 2.0-1.85 (m, 4H), 1.57-1.50 (m, 8H), 1.40-1.0 (m, 42H), 0.9-0.7 (12); APCI-MS: m/z [M+H]⁺ 954.3.

### Synthesis of ((4-(thiazol-2-yl)butyl)azanediyl)bis(heptane-7,1-diyl)bis(4,4-bis(nonyloxy) butanoate) (Compound AT-2)

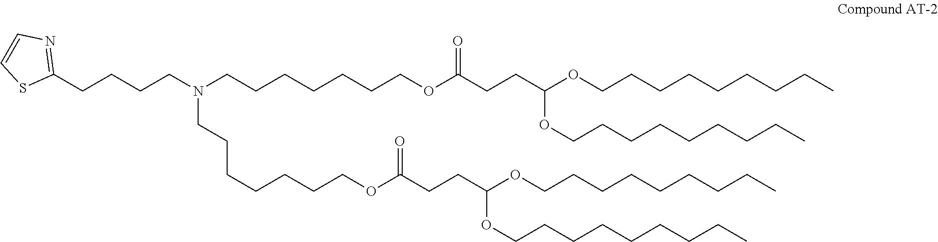 [SMILES: CCCCCCCCCOC(CCC(=O)OCCCCCCCN(CCCCCCCOC(=O)CCC(OCCCCCCCCC)OCCCCCCCCC)CCCCc1nccs1)OCCCCCCCCC]

To a mixture of L74-8 (310 mg, 0.32 mmol) and L74-4 (100.8 mg, 0.65 mmol) in 1,2-dichloroethane (10 mL) was added Na(OAc)₃BH (206.6 mg, 0.97 mmol) and acetic acid (0.02 mL, 0.32 mmol). The reaction mixture was subjected to vacuum/N₂ cycle (×3) and stirred under nitrogen at room temperature for 18 h. The reaction was quenched by slow addition of saturated NaHCO₃ (100 mL) at 0° C. The aqueous phase was extracted using DCM (100 mL×3) and the combined organic phases were dried over anhydrous Na₂SO₄. Filtration followed by concentration provided crude material, which was loaded on 20 g flash silica column and was purified by flash chromatography (SiO₂: hexane/ethyl acetate (0 to 100%)) followed by another column using (DCM:MeOH:NH₄OH (9:1:0.1)) to yield Compound AT-2 (192 mg, 54%) as colorless oil. ¹H-NMR (300 MHz, CDCl₃) δ 7.65 (d, 1H), 7.18 (d, 1H), 4.48 (t, 2H), 4.04 (t, 4H), 3.57-3.37 (m, 8H), 3.06 (t, 2H), 3.39-3.34 (m, 8H), 1.92-1.70 (1, 6H), 1.70-1.44 (m, 12H), 1.44-1.0 (m, 68H), 0.89-0.84 (m, 12H); APCI-MS: m/z [M+H]⁺ 1093.9; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=11.6 min, purity: >99%; UPLC column: Waters Aquity UPLC® CSH™, C18, 1.7 μm, 3.0×150 mmol, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=13.8 min, purity: >99%.

### Synthesis of AT-3

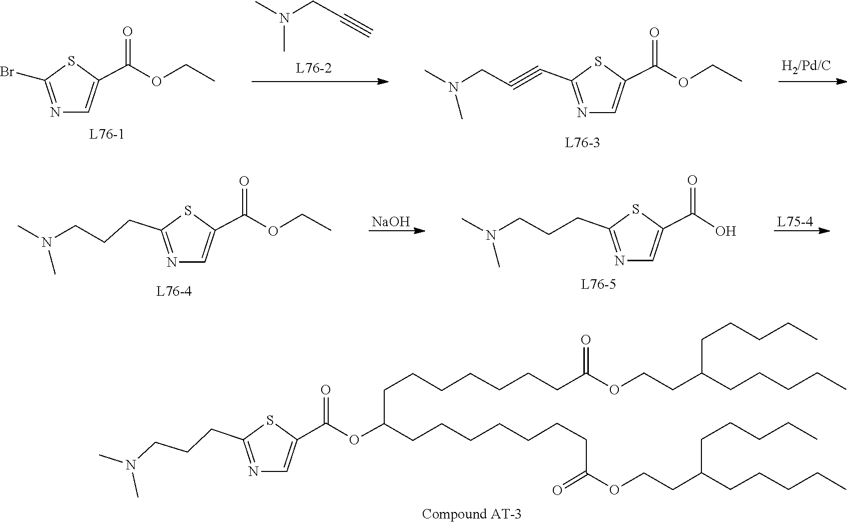

### Synthesis of ethyl 2-(3-(dimethylamino) prop-1-yn-1-yl)thiazole-5-carboxylate (L76-3)

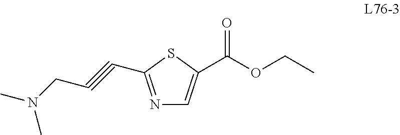 [SMILES: CCOC(=O)c1cnc(C#CCN(C)C)s1]

To the mixture of starting materials L76-1 (3.78 g, 16.01 mmol), L76-2 (2 g, 24.02 mmol), copper (I) iodide (100 mg, 0.48 mmol) and tetrakis(triphenylphosphine) palladium (380 mg, 0.32 mmol) was added triethylamine and THF (60/20 mL). The reaction mixture was then stirred at 90° C. for 20 hours. When TLC (15% MeOH in DCM, R_{f}=0.6) showed the completion of reaction, the solvent was evaporated to obtain crude product which was then subjected to silica gel column using 0-15% MeOH in DCM as eluent to afford L76-3 as red oil (2.84 g, 75%). ¹H-NMR (300 MHz, CDCl₃) δ 8.34 (s, 1H), 4.36 (q, 2H), 3.57 (s, 2H), 2.38 (s, 6H), 1.38 (t, 3H). CIMS m/z [M+H]⁺ 239.1.

### Synthesis of ethyl 2-(3-(dimethylamino)propyl)thiazole-5-carboxylate (L76-4)

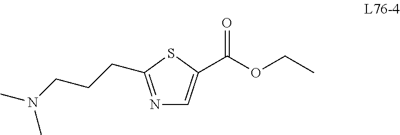 [SMILES: CCOC(=O)c1cnc(CCCN(C)C)s1]

L76-3 (2.84 g, 11.92 mmol) was dissolved in MeOH (100 mL), followed by addition of 20 wt % Pd/C (1 g). The mixture was stirred under H₂ atmosphere at 45° C. for 2 hours. When TLC (10% MeOH in DCM, R_{f}=0.3) showed the completion of reaction, the solvent was evaporated to obtain crude product which was then subjected to silica gel column using 0-10% MeOH in DCM as eluent to afford L76-4 as yellow oil (870 mg, 30%). ¹H-NMR (300 MHz, CDCl₃) δ 8.20 (s, 1H), 4.29 (q, 2H), 3.01 (t, 2H), 2.35 (t, 2H), 2.22 (s, 6H), 2.04-1.88 (m, 2H), 1.32 (t, 3H). CIMS m/z [M+H]⁺ 243.1.

### Synthesis of 2-(3-(dimethylamino)propyl)thiazole-5-carboxylic acid (L76-5)

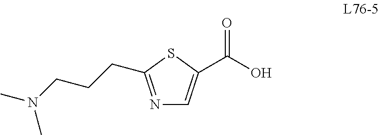

L76-4 (800 mg, 3.30 mmol) and sodium hydroxide (99 mg, 9.90 mmol) were dissolved in MeOH/H₂O (3/3 mL). The reaction mixture was then stirred at room temperature for 20 hours. Solvent was then evaporated. To the crude product was added anhydrous MeOH (5 mL) and insoluble salt was removed by filtration. The organic fraction was evaporated to obtain product L76-5 as yellow semisolid (500 mg, 94%). ¹H-NMR (300 MHz, CD₃OD) δ 8.07 (s, 1H), 3.32-3.28 (m, 2H), 3.16 (t, 2H), 2.95 (s, 6H), 2.37-2.20 (m, 2H). CIMS m/z [M+H]⁺ 215.0.

### Synthesis of bis(3-pentyloctyl) 9-((2-(3-(dimethylamino)propyl)thiazole-5-carbonyl)oxy) heptadecanedioate (Compound AT-3)

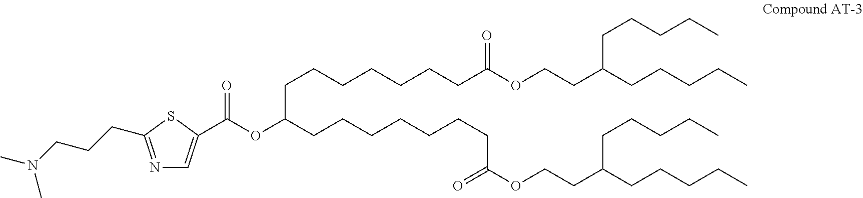 [SMILES: CCCCCC(CCCCC)CCOC(=O)CCCCCCCC(CCCCCCCC(=O)OCCC(CCCCC)CCCCC)OC(=O)c1cnc(CCCN(C)C)s1]

L76-5 (63 mg, 0.26 mmol), EDC (113 mg, 0.52 mmol) and DMAP (20 mg, 0.14 mmol) were dissolved in DCM/DIPEA (5/0.1 mL). After stirring for 5 minutes the solution became clear. L75-4 (100 mg, 0.147 mmol) was then added to the above solution. The reaction mixture was stirred for 20 hours at room temperature. When TLC (15% MeOH in DCM with 1% NH₄OH, R_{f}=0.4) showed the completion of reaction, the solvent was evaporated. The obtained crude product was then subjected to silica gel column using 0-15% MeOH in DCM with 1% NH₄OH as eluent to afford Compound AT-3 as light-yellow oil (71 mg, 56%). ¹H-NMR (300 MHz, CDCl₃) δ 8.24 (s, 1H), 5.04 (quint, 1H), 4.07 (t, 4H), 3.07 (t, 2H), 2.61-2.39 (t, 2H), 2.38-2.19 (m, 10H), 2.02 (quint, 2H), 1.70-1.49 (m, 12H), 1.45-1.05 (m, 50H), 0.87 (t, 12H). CIMS m/z [M+H]⁺ 877.6. Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 40% to 100% in 5 min, then 100% for 15 min. Flow rate: 1 mL/min, column temperature: 20±2° C., detector: ELSD, t_{R}=6.7 min, purity: >99%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=7.38 min, purity: 99.7%.

### Synthesis of AT-4

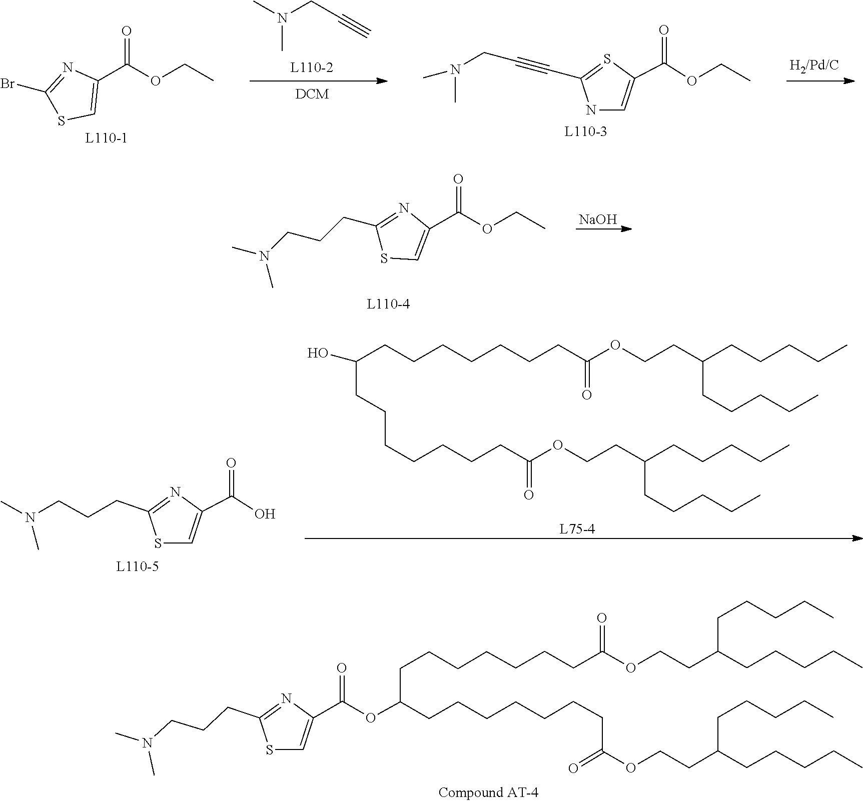

### Synthesis of ethyl 2-(3-(dimethylamino) prop-1-yn-1-yl)thiazole-4-carboxylate (L110-3)

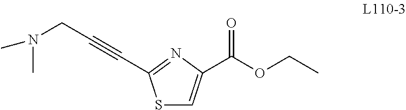 [SMILES: CCOC(=O)c1csc(C#CCN(C)C)n1]

To a solution of L110-1 (5.16 g, 21.86 mmol) in THF (20 mL) and triethylamine (60 mL), was added L110-2 (2.47 g, 29.71 mmol), CuI (1.24 g, 0.66 mmol) and Pd(PPh₃)₄ (505 mg, 0.44 mmol). The mixture was stirred at 90° C. overnight and concentrated under reduced pressure. The crude material was purified using flash chromatography (SiO₂: 0-10% methanol in dichloromethane gradient) to afford 1.1 g of L110-3. The impure fractions were further purified using flash chromatography (SiO₂: 0-100% ethyl acetate in hexanes gradient) to afford 1.8 g of L110-3. Both batches of the product were combined to afford L110-3 as brown solid (2.9 g, 56%). ¹HNMR (300 MHz, CDCl₃) δ 8.14 (s, 1H), 4.43 (q, J=7.2 Hz, 2H), 3.54 (s, 2H), 2.37 (s, 6H), 1.41 (1, J=7.0 Hz, 3H). CIMS m/z [M+H]⁺ 239.

### Synthesis of ethyl 2-(3-(dimethylamino)propyl)thiazole-4-carboxylate (L110-4)

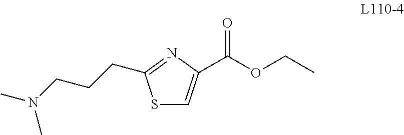 [SMILES: CCOC(=O)c1csc(CCCN(C)C)n1]

A mixture of L110-3 (2.25 g, 9.44 mmol) and 10% Pd/C (2.25 g) in methanol (10 mL) was stirred at room temperature under a hydrogen balloon for 1 h. The reaction mixture was filtered through celite and concentrated under reduced pressure. The crude material was purified using column chromatography (SiO₂: 0-20% methanol, 0-1% ammonium hydroxide in dichloromethane gradient) to afford L110-4 as a yellow oil (1.4 g, 64%). ¹HNMR (300 MHz, CDCl₃) δ 8.05 (s, 1H), 4.41 (d, J=7.2 Hz, 2H), 3.09 (t, J=7.8 Hz, 2H), 2.33 (t, J=7.3 Hz, 2H), 2.22 (s, 6H), 1.97 (d, J=7.4 Hz, 2H), 1.39 (t, J=7.2 Hz, 3H). CIMS m/z [M+H]⁺ 243.1.

### Synthesis of 2-(3-(dimethylamino)propyl)thiazole-4-carboxylic acid (L110-5)

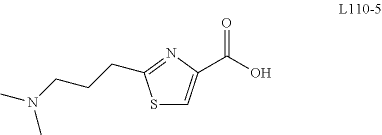

A mixture of L110-4 (1.46 g, 6.02 mmol) and 1N NaOH (6.5 mL) in methanol (20 mL) was stirred at room temperature overnight. The pH of the reaction mixture was adjusted to pH 3 by adding 1N HCl dropwise with ice bath cooling. Concentration under reduced pressure gave a residue which was further dried by co-evaporation with toluene (20 mL×4) under reduced pressure. Crude L110-5 (1.80 g) was obtained as a brownish solid which was used for the next step reaction without further purification. ¹HNMR (300 MHz, DMSO-d₆) δ 8.16 (s, 1H), 3.07 (t, J=7.3 Hz, 2H), 2.90 (d, J=8.0 Hz, 2H), 2.62 (s, 6H), 2.51 (t, J=1.7 Hz, 2H), 2.09 (m, J=8.0 Hz, 2H).

### Synthesis of bis(3-pentyloctyl) 9-((2-(3-(dimethylamino)propyl)thiazole-4-carbonyl)oxy) heptadecanedioate (Compound AT-4)

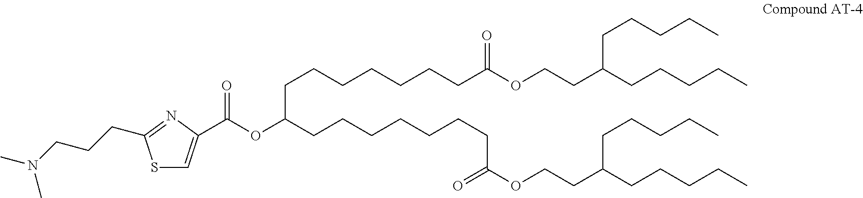 [SMILES: CCCCCC(CCCCC)CCOC(=O)CCCCCCCC(CCCCCCCC(=O)OCCC(CCCCC)CCCCC)OC(=O)c1csc(CCCN(C)C)n1]

To a solution of L110-5 (189 mg, 0.88 mmol) in DCM (6 mL), DMAP (36 mg, 0.29 mmol), DIPEA (76 mg, 0.59 mmol) and EDC (338 mg, 1.76 mmol) were added. The mixture was left to stir for 15 min before L75-4 (200 mg, 0.29 mmol) was added. The reaction mixture was stirred at room temperature overnight. Concentration gave a residue which was purified using flash chromatography (SiO₂: 0-5% methanol, 0-0.5% ammonium hydroxide in dichloromethane gradient) to afford Compound AT-4 as a yellow oil (218 mg, 84%); ¹HNMR (300 MHz, CDCl₃) δ 8.03 (s, 1H), 5.18-5.14 (m, 1H), 4.10 (t, J=7.0 Hz, 4H), 3.13 (t, J=7.7 Hz, 2H), 2.48 (t, J=2.9 Hz, 2H), 2.36-2.26 (m, 8H), 2.10-2.03 (m, 2H), 1.69-1.55 (m, 14H), 1.36-1.27 (m, 50H), 0.90 (t, J=6.7 Hz, 12H); CIMS m/z [M+H]⁺ 877.7; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.6 min, purity: >99%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: CAD, t_{R}=14.5 min, purity: >99%.

### Synthesis of AT-5

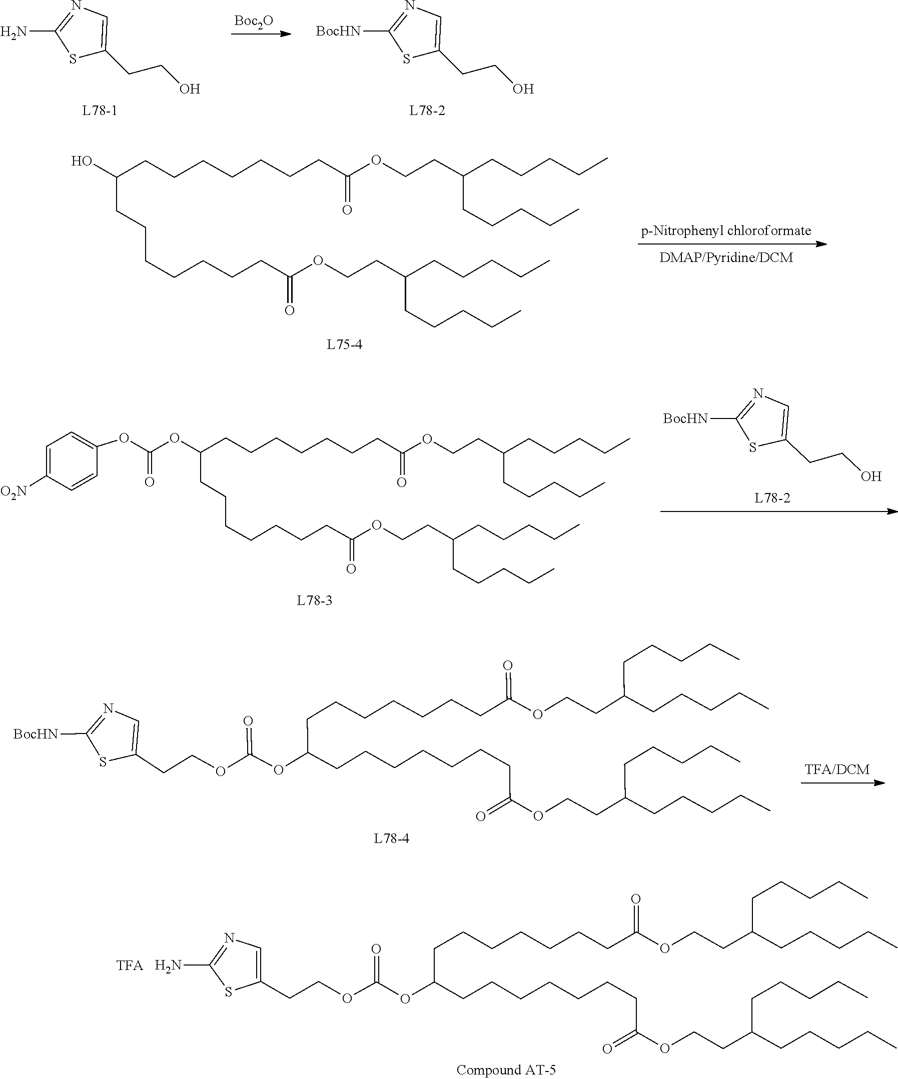

### Synthesis of tert-butyl (5-(2-hydroxyethyl) thiazol-2-yl) carbamate (L78-2)

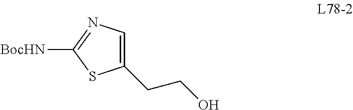 [SMILES: CC(C)(C)OC(=O)Nc1ncc(CCO)s1]

A solution of L78-1 (288 mg, 2.0 mmol) in ethyl acetate (5 mL) was cooled to 0° C. under nitrogen. Di-tert-butyl decarbonate (470 mg, 2.2 mmol) were added and the reaction mixture was stirred at room temperature for 72 h. Concentration provided crude material which was purified by flash column chromatography (SiO₂: ethyl acetate/hexane 0-80%) to yield L78-2 as light-yellow solid (380 mg, 78%); ¹H-NMR (300 MHz, CDCl₃) δ 10.76 (s, br, 1H), 7.11 (s, 1H), 3.83 (m, 2H), 2.98 (t, J=5.1 Hz, 2H), 1.56 (s, 9H); CIMS m/z [M+H]⁺ 245.

### Synthesis of bis(3-pentyloctyl) 9-(((4-nitrophenoxy) carbonyl)oxy) heptadecanedioate (L78-3)

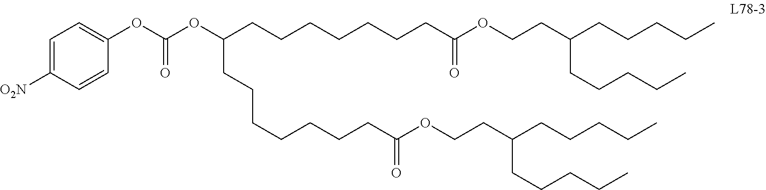 [SMILES: CCCCCC(CCCCC)CCOC(=O)CCCCCCCC(CCCCCCCC(=O)OCCC(CCCCC)CCCCC)OC(=O)Oc1ccc([N+](=O)[O-])cc1]

A solution of L75-4 (800 mg, 1.2 mmol), pyridine (0.3 mL) and DMAP (70 mg, 0.57 mmol) in anhydrous dichloromethane (10 mL) was cooled to 0° C. under nitrogen. 4-nitrophenyl-chloroformate (472 mg, 2.4 mmol) was added. The reaction mixture was stirred at room temperature for 16 h to form a colorless clear solution of L78-3 which was used for the next step without further purification; CIMS m/z [M+H]⁺ 846.6.

### Synthesis of bis(3-pentyloctyl) 9-(((2-(2-((tert-butoxycarbonyl)amino) thiazol-5-yl) ethoxy) carbonyl)oxy) heptadecanedioate (L78-4)

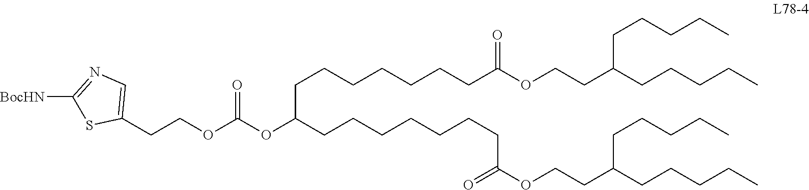 [SMILES: CCCCCC(CCCCC)CCOC(=O)CCCCCCCC(CCCCCCCC(=O)OCCC(CCCCC)CCCCC)OC(=O)OCCc1cnc(NC(=O)OC(C)(C)C)s1]

To half of the above solution of L78-3 in dichloromethane was added DIPEA (120 mg, 0.5 mmol) and a solution of thiazole alcohol L78-2 (120 mg, 0.5 mmol) in dichloromethane (3 mL) under nitrogen atmosphere. The resulting mixture was stirred at room temperature for 18 h. After concentration, the residue was purified by flash column chromatography (SiO₂: ethyl acetate/hexane 0-100%) to yield L78-4 as colorless oil (100 mg, 21%); ¹H NMR (300 MHz, CDCl₃) δ 10.13 (s, br, 1H), 7.10 (s, 1H), 4.65 (m, 1H), 4.29 (t, J=7.0 Hz, 2H), 4.07 (t, J=7.1 Hz, 4H), 3.09 (t, J=6.8 Hz, 2H), 2.27 (t, J=7.4 Hz, 4H), 1.69-1.43 (m, 21H), 1.42-1.05 (m, 50H), 0.88 (t, J=7.1 Hz, 12H); CIMS m/z [M+H]⁺ 951.

### Synthesis of bis(3-pentyloctyl) 9-(((2-(2-aminothiazol-5-yl) ethoxy) carbonyl)oxy) heptadecanedioate trifluoroacetate (Compound AT-5)

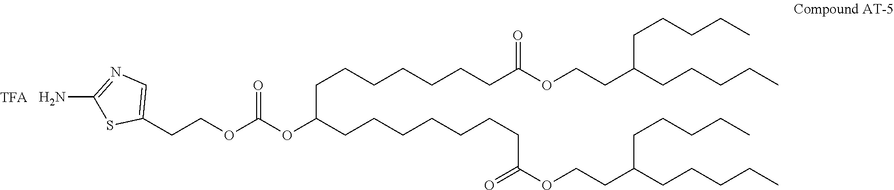 [SMILES: CCCCCC(CCCCC)CCOC(=O)CCCCCCCC(CCCCCCCC(=O)OCCC(CCCCC)CCCCC)OC(=O)OCCc1cnc(N)s1]

To a solution of L78-4 (100 mg, 0.105 mmol) in dichloromethane (1.5 mL) was added TFA (1.5 mL) at 0° C. and the reaction mixture was stirred at room temperature for 12 h. The volatile components were removed under reduced pressure and the residue was co-evaporated three times with methanol and toluene. Drying in high vacuum oven overnight yielded Compound AT-5 as colorless oil (98 mg, 99%); ¹H-NMR (300 MHz, CDCl₃) δ 9.09 (s, br, 2H), 6.80 (s, 1H), 4.80 (m, 1H), 4.28 (t, J=7.0 Hz, 2H), 4.07 (t, J=7.1 Hz, 4H), 2.98 (t, J=6.8 Hz, 2H), 2.28 (t, J=7.4 Hz, 4H), 1.69-1.48 (m, 12H), 1.49-1.15 (m, 50H), 0.88 (t, J=7.1 Hz, 12H); CIMS m/z [M+H]⁺ 851.6; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=8.4 min, purity: >99%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=15.8 min, purity: >99%.

### Synthesis of AT-6

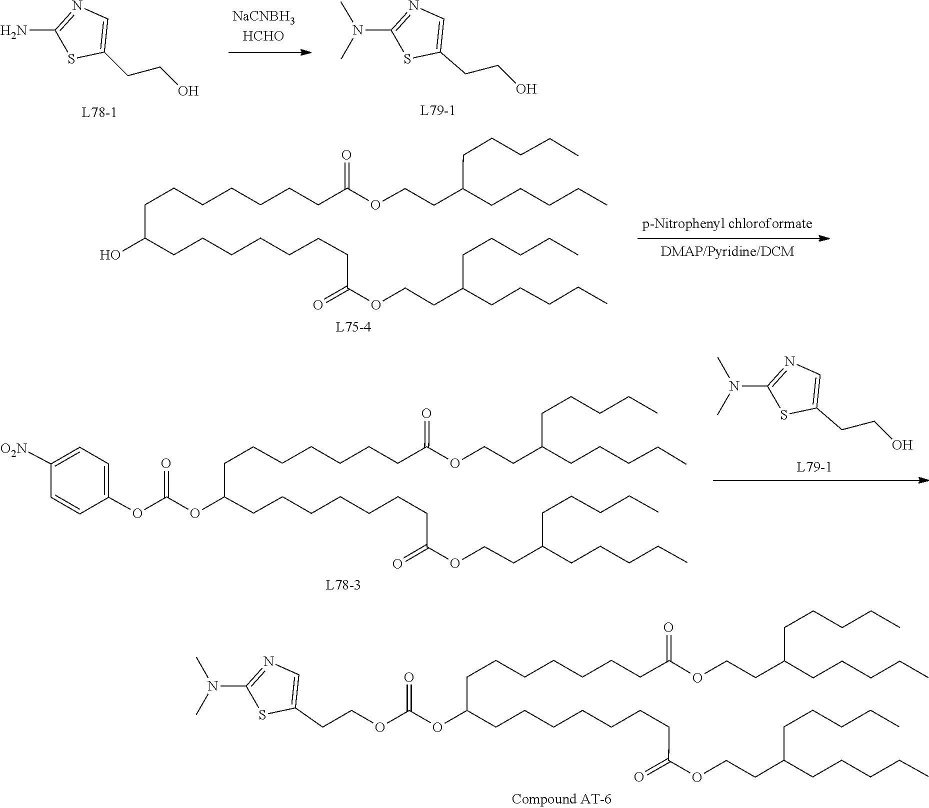

### Synthesis of 2-(2-(dimethylamino) thiazol-5-yl) ethan-1-ol (L79-1)

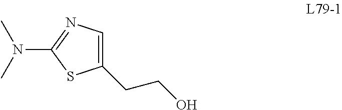

A solution of L78-1 (1.2 g, 8.3 mmol) and paraformaldehyde (9.98 g, 332.9 mmol) in THF (40 mL) was heated to weak reflux for 1 h. After cooling to room temperature, sodium cyanoborohydride (10.5 g, 166.4 mmol) was added in. The resulting mixture was refluxed under nitrogen for 48 h and then concentrated under reduced pressure. To the residue was added acetone (30 mL) and HCl (1M, 10 mL) and the reaction mixture was stirred at room temperature for 48 h. With ice-bath cooling, the reaction mixture was neutralized with saturated aq. sodium bicarbonate solution. Concentration under reduced pressure yielded crude product which was purified by flash chromatography (SiO₂: DCM/MeOH 0-10%) to give L79-1 (300 mg, 21%); ¹HNMR (CDCl₃) δ 3.90 (s, 1H), 3.77 (t, J=7.0 Hz, 2H), 3.05 (s, 6H), 2.88 (t, J=7.0 Hz, 2H), 1.38-1.24 (m, 20H), 0.87 (t, J=7.0 Hz, 6H); CIMS m/z [M+H]⁺ 173.

### Synthesis of bis(3-pentyloctyl) 9-(((4-nitrophenoxy) carbonyl)oxy) heptadecanedioate (L78-3)

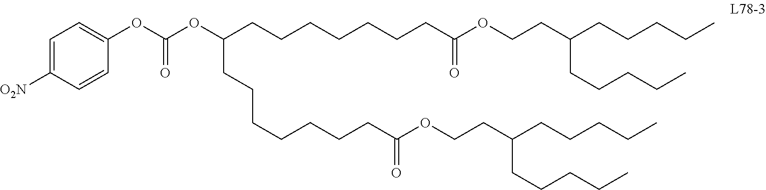 [SMILES: CCCCCC(CCCCC)CCOC(=O)CCCCCCCC(CCCCCCCC(=O)OCCC(CCCCC)CCCCC)OC(=O)Oc1ccc([N+](=O)[O-])cc1]

A solution of L75-4 (500 mg, 0.73 mmol), pyridine (232 mg, 2.9 mmol) and DMAP (45 mg, 0.36 mmol) in dichloromethane (10 mL) was cooled to 0° C. under nitrogen. 4-Nitrophenyl-chloroformate (295 mg, 1.46 mmol) was added, and the mixture was stirred at room temperature for 40 h. The reaction mixture was diluted with DCM (15 mL) and washed with water (15 mL) and brine (15 mL). The organic phase was dried over anhydrous Na₂SO₄. Filtration and concentration provided crude material which was purified by flash column chromatography (SiO₂: ethyl acetate/hexane 0-100%) to yield L78-3 as colorless oil (505 mg, 81%); ¹H-NMR (300 MHz, CDCl₃) δ 8.27 (d, 2H), 7.38 (d, 2H), 4.82 (m, 1H), 4.07 (t, J=7.1 Hz, 4H), 2.28 (t, J=7.7 Hz, 4H), 1.73-1.48 (m, 12H), 1.47-1.11 (m, 50H), 0.88 (t, J=7.1 Hz, 12H); CIMS m/z [M+H]⁺ 846.

### Synthesis of bis(3-pentyloctyl) 9-(((2-(2-(dimethylamino) thiazol-5-yl) ethoxy) carbonyl)oxy) heptadecanedioate (Compound AT-6)

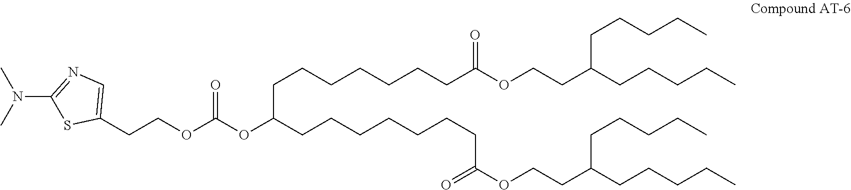 [SMILES: CCCCCC(CCCCC)CCOC(=O)CCCCCCCC(CCCCCCCC(=O)OCCC(CCCCC)CCCCC)OC(=O)OCCc1cnc(N(C)C)s1]

To a solution of L78-3 (500 mg, 0.59 mmol) in dichloromethane (7 mL) was added a solution of L79-1 (120 mg, 0.70 mmol) in dichloromethane (3 mL), DMAP (60 mg, 0.5 mmol) and DIPEA (380 mg, 2.9 mmol). The resulting mixture was stirred at room temperature for 20 h. After concentration, the residue was purified by flash column chromatography (SiO₂: ethyl acetate/hexane 0-100%) to yield Compound AT-6 as colorless oil (410 mg, 79%); ¹H-NMR (300 MHz, CDCl₃) δ 6.90 (s, 1H), 4.66 (m, 1H), 4.25 (t, J=7.0 Hz, 2H), 4.07 (t, J=7.1 Hz, 4H), 3.06 (s, 6H), 3.01 (t, J=6.9 Hz, 2H), 2.27 (t, J=7.4 Hz, 4H), 1.69-1.48 (m, 12H), 1.46-1.15 (m, 50H), 0.88 (t, J=7.1 Hz, 12H); CIMS m/z [M+H]⁺ 879.6; Analytical HPLC column: Agela Durashell C18, 3 μm (Catalog No. DC930505-0), 4.6×150 mm, mobile phase A: acetonitrile with 0.1% trifluoroacetic acid, mobile phase B: water with 0.1% trifluoroacetic acid, use gradient: A in B 5% to 95% in 15 min, flow rate: 1 mL/min, column temperature: 20±2° C., detector: ELSD, t_{R}=6.9 min, purity: >99%; UPLC column: Waters Aquity UPLC® CSH™, C18, 1.7 μm, 3.0×150 mm, (Part No. 186005302), mobile phase A: acetonitrile with 0.1% trifluoroacetic acid, mobile phase B: water with 0.1% trifluoroacetic acid, use gradient: A in B 60% to 100% in 15 min, flow rate: 0.5 mL/min, column temperature: 20±2° C., detector: CAD, t_{R}=11.0 min, purity: >99%.

### Example 2A: LNP Formulations A-Cre mRNA Formulations

Ionizable lipids, DSPC, cholesterol, and a PEG lipid were dissolved in pure ethanol at the specified mol % ratios with a total lipid concentration of 10.8 mM. A 0.10 mg/mL mRNA solution was prepared using acidic buffer (pH 4.0-5.0) containing mRNA encoding Cre recombinase (SEQ ID NO: 47). The nucleotide and lipid solutions were mixed at a 3:1 volume ratio using the NANOASSEMBLR microfluidic system at a 12 mL/min total flow rate resulting in rapid mixing and self-assembly of LNPs. Formulations were further dialyzed against PBS (pH 7.4) overnight at 4° C., concentrated using centrifugal filtration and filtered (0.2 μm pore size). The particle size and polydispersity index (PDI) of formulations was measured by dynamic light scattering (DLS) using a ZETASIZER ULTRA (Malvern Panalytical). RNA encapsulation efficiency (EE %) was determined by Ribogreen assay.

TABLE 2A
LNP Formulations

| | | | | | Size (Z- | | | |
|---|---|---|---|---|---|---|---|---|
| | IL | PL | SL | PEG-L | avg | | EE | |
| Form | (mol %) | (mol %) | (mol %) | (mol %) | nm) | PDI | (%) | Buffer |
| F-1 | C66 | DSPC | Chol | DSPE-PEG2k | 178.9 | 0.09 | 89.7 | Y |
| | (48.5%) | (10%) | (40.5%) | (1.0%) | | | | |
| F-2 | C66 | DSPC | Chol | DSPE-PEG2k | 173.9 | 0.05 | 88.7 | Y |
| | (48.5%) | (10%) | (40.5%) | (1.0%) | | | | |
| F-3 | CY63 | DSPC | Chol | DSPE-PEG2k | 170.2 | 0.22 | 97.2 | Y |
| | (48.5%) | (10%) | (40.5%) | (1.0%) | | | | |
| F-4 | CY63 | DSPC | Chol | DSPE-PEG2k | 132.3 | 0.04 | 94.2 | Y |
| | (48.5%) | (10%) | (40.5%) | (1.0%) | | | | |
| F-5 | CY70 | DSPC | Chol | DSPE-PEG2k | 133.2 | 0.002 | 94.9 | Y |
| | (48.5%) | (10%) | (40.5%) | (1.0%) | | | | |
| F-6 | CY70 | DSPC | Chol | DSPE-PEG2k | 129.3 | 0.05 | 96.3 | Y |
| | (48.5%) | (10%) | (40.5%) | (1.0%) | | | | |
| F-7 | C66 | DSPC | Chol | DSPE-PEG2k | 102.3 | 0.02 | 96.0 | Y |
| | (48.5%) | (10%) | (40%) | (1.5%) | | | | |
| F-8 | C72 | DSPC | Chol | DSPE-PEG2k | 90.1 | 0.02 | 95.4 | X |
| | (48.5%) | (10%) | (40%) | (1.5%) | | | | |
| F-9 | C73 | DSPC | Chol | DSPE-PEG2k | 86.1 | 0.01 | 96.2 | X |
| | (48.5%) | (10%) | (40%) | (1.5%) | | | | |
| F-10 | CY63 | DSPC | Chol | DSPE-PEG2k | 113.4 | 0.07 | 95.6 | Y |
| | (48.5%) | (10%) | (40.5%) | (1.0%) | | | | |
| F-11 | CY67 | DSPC | Chol | DSPE-PEG2k | 128 | 0.08 | 96.6 | Y |
| | (48.5%) | (10%) | (40.5%) | (1.0%) | | | | |
| F-12 | CY71 | DSPC | Chol | DSPE-PEG2k | 116.5 | 0.06 | 98.2 | Y |
| | (48.5%) | (10%) | (40.5%) | (1.0%) | | | | |
| F-13 | C66 | DSPC | Chol | DSPE-PEG2k | 95.7 | 0.02 | 95.7 | Y |
| | (48.5%) | (10%) | (40%) | (1.5%) | | | | |
| F-14 | C61 | DSPC | Chol | DSPE-PEG2k | 97.5 | 0.02 | 95.3 | Y |
| | (48.5%) | (10%) | (40.5%) | (1.0%) | | | | |
| F-15 | C74 | DSPC | Chol | DSPE-PEG2k | 164.7 | 0.04 | 92.7 | Y |
| | (48.5%) | (10%) | (40.5%) | (1.0%) | | | | |
| F-16 | C66 | DSPC | Chol | DSPE-PEG2k | 92.5 | 0.03 | 91.4 | Y |
| | (48.5%) | (10%) | (40%) | (1.5%) | | | | |
| F-17^{a} | C66 | DSPC | Chol | DSPE-PEG2k | 119.8 | 0.06 | 92.7 | Y |
| | (48.5%) | (10%) | (40%) | (1.5%) | | | | |
| F-18 | C66 | DSPC | Chol | DSPE-PEG2k | 108.6 | 0.004 | 92.9 | Y |
| | (48.5%) | (10%) | (39.5%) | (2.0%) | | | | |
| F-19 | C66 | DSPC | Chol | DSPE-PEG2k:DMG- | 92.5 | 0.01 | 96.9 | Y |
| | (48.5%) | (10%) | (39%) | PEG2k | | | | |
| | | | | (1.0%:1.5%) | | | | |
| F-20 | C66 | DSPC | Chol | DSPE-PEG2k:DMG- | 70.8 | 0.03 | 95.4 | Y |
| | | | | PEG2k | | | | |
| | (48.5%) | (10%) | (39%) | (1.5%:1.0%) | | | | |
| F-21 | C66 | DSPC | Chol | DSPE-PEG2k | 79.1 | 0.02 | 95.4 | X |
| | (48.5%) | (10%) | (40%) | (1.5%) | | | | |
| F-22^{b} | C57 | DSPC | Chol | DSPE-PEG2k | 79.4 | 0.03 | 92.3 | X |
| | (48.5%) | (10%) | (40%) | (1.5%) | | | | |
| F-23 | C78 | DSPC | Chol | DSPE-PEG2k | 84.8 | 0.04 | 82.4 | X |
| | (48.5%) | (10%) | (40%) | (1.5%) | | | | |
| F-27 | C66 | DSPC | Chol | DSPE-PEG2k | 95.71 | 0.078 | 97.3 | Y |
| | (48.5%) | (10%) | (39.5%) | (2.0%) | | | | |
| F-28 | C66 | DSPC | Chol | DSG-PEG2k | 84.53 | 0.137 | 92 | X |
| | (48.5%) | (10%) | (39.5%) | (2.0%) | | | | |
| F-29 | C66 | DSPC | Chol | PL-12A | 108.2 | 0.153 | 82.1 | X |
| | (48.5%) | (10%) | (39.5%) | (2.0%) | | | | |
| F-30 | C66 | DSPC | Chol | PL-10A | 88.8 | 0.138 | 93.8 | X |
| | (48.5%) | (10%) | (39.5%) | (2.0%) | | | | |
| F-31 | C66 | DSPC | Chol | PL-9A | 103.8 | 0.092 | 82.6 | X |
| | (48.5%) | (10%) | (39.5%) | (2.0%) | | | | |
| F-32 | C66 | DOPE | Chol | DSPE-PEG2k | 87.03 | 0.33 | 93.9 | Y |
| | (48.5%) | (10%) | (39.5%) | (2.0%) | | | | |
| F-33 | C66 | DOPC | Chol | DSPE-PEG2k | 83.07 | 0.107 | 95.7 | Y |
| | (48.5%) | (10%) | (39.5%) | (2.0%) | | | | |
| F-34^{b} | C57 | DSPC | Chol | DSPE-PEG2k | 84.61 | 0.056 | 97.9 | Y |
| | (48.5%) | (10%) | (39.5%) | (2.0%) | | | | |
| F-35 | C66 | DLPC | Chol | DSPE-PEG2k | 74.85 | 0.043 | 95.7 | Y |
| | (48.5%) | (10%) | (39.5%) | (2.0%) | | | | |
| F-36 | C62 | DSPC | Chol | DSPE-PEG2k | 90.79 | 0.053 | 98.4 | Y |
| | (48.5%) | (10%) | (39.5%) | (2.0%) | | | | |
| F-37 | C56 | DSPC | Chol | DSPE-PEG2k | 80.31 | 0.082 | 95.3 | X |
| | (48.5%) | (10%) | (39.5%) | (2.0%) | | | | |
| F-38 | C59 | DSPC | Chol | DSPE-PEG2k | 111.5 | 0.014 | 96.2 | X |
| | (48.5%) | (10%) | (39.5%) | (2.0%) | | | | |
| F-39 | C54 | DSPC | Chol | DSPE-PEG2k | 104.6 | 0.126 | 90.2 | X |
| | (48.5%) | (10%) | (39.5%) | (2.0%) | | | | |
| F-40 | C80 | DSPC | Chol | DSPE-PEG2k | 97.81 | 0.057 | 96.6 | X |
| | (48.5%) | (10%) | (39.5%) | (2.0%) | | | | |
| F-41 | C79 | DSPC | Chol | DSPE-PEG2k | 80.47 | 0.076 | 97.1 | X |
| | (48.5%) | (10%) | (39.5%) | (2.0%) | | | | |
| F-42 | C76 | DSPC | Chol | DSPE-PEG2k | 85.23 | 0.071 | 94.8 | X |
| | (48.5%) | (10%) | (39.5%) | (2.0%) | | | | |
| F-43 | C77 | DSPC | Chol | DSPE-PEG2k | 82.1 | 0.065 | 96.4 | X |
| | (48.5%) | (10%) | (39.5%) | (2.0%) | | | | |
| F-44 | C78 | DSPC | Chol | DSPE-PEG2k | 91.27 | 0.058 | 93.5 | Y |
| | (48.5%) | (10%) | (39.5%) | (2.0%) | | | | |
| F-70 | C66 | DSPC | Chol | DSPE-PEG2k | 83.03 | 0.043 | 97.4 | X |
| | (48.5%) | (10%) | (40%) | (1.5%) | | | | |
| F-71# | C66 | DSPC | Chol | DSPE-PEG2k | 90.04 | 0.057 | 96.8 | X |
| | (48.5%) | (10%) | (40%) | (1.5%) | | | | |

^{a}N:P = 9;
^{b}N:P = 8;
unless otherwise specified all other formulations N:P = 6
Buffer X: 25 mM Sodium Acetate, pH 5.0;
Buffer Y: 50 mM Citrate, pH 4.0
#Payload = fLUC mRNA instead of cre Recombinase; otherwise identical procedure

### Example 2B: LNP Formulations B—VHH Formulations

Ionizable lipids, DSPC, cholesterol, and a PEG lipid were dissolved in pure ethanol at the specified mol % ratios with a total lipid concentration of 10.8 mM. A 0.10 mg/mL mRNA solution was prepared using acidic buffer (pH 4.0-5.0) containing mRNA encoding VHH (SEQ ID NO: 48). The nucleotide and lipid solutions were mixed at a 3:1 volume ratio using the NANOASSEMBLR microfluidic system at a 12 mL/min total flow rate resulting in rapid mixing and self-assembly of LNPs. Formulations were further dialyzed against PBS (pH 7.4) overnight at 4° C., concentrated using centrifugal filtration and filtered (0.2 μm pore size). The particle size and polydispersity index (PDI) of formulations was measured by dynamic light scattering (DLS) using a ZETASIZER ULTRA (Malvern Panalytical). RNA encapsulation efficiency (EE %) was determined by Ribogreen assay.

TABLE 2B
LNP Formulations

| | IL | PL | SL | PEG-L | Size (Z- | | EE | |
|---|---|---|---|---|---|---|---|---|
| Form | (mol %) | (mol %) | (mol %) | (mol %) | avg nm) | PDI | (%) | Buffer |
| F-24 | C66 | DSPC | Chol | DSPE-PEG2k | 93.0 | 0.09 | 98.4 | Y |
| | (48.5%) | (10%) | (39.5%) | (2.0%) | | | | |
| F-25 | C66 | DSPC | Chol | DSPE-PEG2k:DMG- | 107.9 | 0.08 | 88.3 | Y |
| | (48.5%) | (10%) | (39%) | PEG2k | | | | |
| | | | | (1.0%:1.5%) | | | | |
| F-26 | C78 | DSPC | Chol | DSPE-PEG2k | 85.23 | 0.07 | 93.2 | X |
| | (48.5%) | (10%) | (39.5%) | (2.0%) | | | | |
| F-45 | C66 | DOPC | Chol | DSG-PEG2k | 94.02 | 0.062 | 94.2 | Y |
| | (48.5%) | (10%) | (39.5%) | (2.0%) | | | | |
| F-46 | C66 | DOPC | Chol | PL-9A | 122.0 | 0.075 | 92.4 | Y |
| | (48.5%) | (10%) | (39.5%) | (2.0%) | | | | |
| F-47^{b} | C57 | DSPC | Chol | PL-9A | 101.9 | 0.141 | 91.4 | X |
| | (48.5%) | (10%) | (39.5%) | (2.0%) | | | | |
| F-48 | C62 | DSPC | Chol | PL-9A | 82.22 | 0.078 | 93.6 | X |
| | (48.5%) | (10%) | (39.5%) | (2.0%) | | | | |
| F-49 | C59 | DSPC | Chol | PL-9A | 105.4 | 0.057 | 92 | X |
| | (48.5%) | (10%) | (39.5%) | (2.0%) | | | | |
| F-50 | C80 | DSPC | Chol | PL-9A | 96.11 | 0.241 | 96.9 | X |
| | (48.5%) | (10%) | (39.5%) | (2.0%) | | | | |
| F-51 | C76 | DSPC | Chol | PL-9A | 78.51 | 0.057 | 96.9 | X |
| | (48.5%) | (10%) | (39.5%) | (2.0%) | | | | |
| F-72 | C66 | DSPC | Chol | DSPE-PEG2k | 96.3 | 0.035 | 95.3 | X |
| | (48.5%) | (10%) | (39.5%) | (2.0%) | | | | |
| F-73 | C62 | DSPC | Chol | DSPE-PEG2k | 98.9 | 0.09 | 95.4 | X |
| | (48.5%) | (10%) | (39.5%) | (2.0%) | | | | |
| F-74 | C59 | DSPC | Chol | DSPE-PEG2k | 123.5 | 0.047 | 95.4 | Y |
| | (48.5%) | (10%) | (39.5%) | (2.0%) | | | | |
| F-75 | C66 | DSPC | Chol | PL-9A | 101.5 | 0.04 | 96 | X |
| | (48.5%) | (10%) | (39.5%) | (2.0%) | | | | |
| F-85^{b} | AX-6 | ESM | Chol | DMG-PEG2k | 78.7 | 0.046 | 95.2 | Y |
| | (33%) | (40%) | (25%) | (2.0%) | | | | |
| F-86^{b} | AX-6 | ESM + DSPC | Chol | DMG-PEG2k | 79.0 | 0.010 | 97.7 | Y |
| | (33%) | (20 + 20%) | (25%) | (2.0%) | | | | |

Buffer X: 25 mM Sodium Acetate, pH 5.0;
Buffer Y: 50 mM Citrate, pH 4.0
a - N:P = 9;
^{b}N:P = 8;
unless otherwise specified all other formulations N:P = 6; AX-6: an ionizable lipid comprising a protonatable tertiary amine head group and at least two biodegradable linkers

### Example 2C: LNP Formulations C—General Editing Formulations

Ionizable lipids, phospholipids, cholesterol, and PEG-lipids are dissolved in pure ethanol at specified molar ratios (exemplary formulations shown below as Formulation Molar Ratios A, B, C, and D), with a total lipid concentration of about 7.2 mM. A polynucleotide solution (exemplary concentration of 0.067 mg/mL) is prepared using acidic buffer (pH 4.0-5.0) containing a gene editing system, such as a 1:1 ratio of a Cas9 mRNA/gRNA or Cas12a mRNA/gRNA system as disclosed in any one of the references in Table X1 (including but not limited to those described in US20210180091A1 or US20210254061A1). The nucleotide and lipid solutions are mixed at a 3:1 volume ratio using a NANOASSEMBLR microfluidic system at a 12 mL/min total flow rate resulting in rapid mixing and self-assembly of LNPs. Formulations are further dialyzed against PBS (pH 7.4) overnight at 4° C., concentrated using centrifugal filtration and filtered (0.2 μm pore size). The particle size and polydispersity index (PDI) of formulations was measured by dynamic light scattering (DLS) using a ZETASIZER ULTRA (Malvern Panalytical). RNA encapsulation efficiency (EE %) was determined by Ribogreen assay.

### Formulation Molar Ratio A:

| Ionizable Lipid | DSPC | Cholesterol | DMG PEG-2k |
|---|---|---|---|
| 48.5 | 10 | 39 | 2.5 |

### Formulation Molar Ratio B:

| Ionizable Lipid | DSPC | Cholesterol | DMG PEG-2k |
|---|---|---|---|
| 55 | 10 | 32.5 | 2.5 |

### Formulation Molar Ratio C:

| Ionizable Lipid | DSPC | Cholesterol | DMPE PEG-2k |
|---|---|---|---|
| 55 | 10 | 32.5 | 2.5 |

### Formulation Molar Ratio D:

| Ionizable Lipid | DSPC | Cholesterol | DSPE PEG-2k |
|---|---|---|---|
| 48.5 | 10 | 40 | 1.5 |

### Example 2D: LNP Formulations D-Cas9+BCL11A gRNA

Ionizable lipids, DSPC, cholesterol, and a PEG lipid were dissolved in pure ethanol at the specified mol % ratios with a total lipid concentration of 10.8 mM. A 0.10 mg/mL mRNA solution was prepared using acidic buffer (pH 4.0-5.0) containing mRNA encoding Cas9 mRNA (SEQ ID NO: 50) and gRNA targeting BCL11a erythroid enhancer (SEQ ID NO: 49) in a 1:1 molar ratio. The nucleotide and lipid solutions were mixed at a 3:1 volume ratio using the NANOASSEMBLR microfluidic system at a 12 mL/min total flow rate resulting in rapid mixing and self-assembly of LNPs. Formulations were further dialyzed against PBS (pH 7.4) overnight at 4° C., concentrated using centrifugal filtration and filtered (0.2 μm pore size). The particle size and polydispersity index (PDI) of formulations was measured by dynamic light scattering (DLS) using a ZETASIZER ULTRA (Malvern Panalytical). RNA encapsulation efficiency (EE %) was determined by Ribogreen assay.

TABLE 2D
LNP Formulations

| | | | | | Size (Z- | | | |
|---|---|---|---|---|---|---|---|---|
| | IL | PL | SL | PEG-L | avg | | EE | |
| Form | (mol %) | (mol %) | (mol %) | (mol %) | nm) | PDI | (%) | Buffer |
| F-52 | C66 | DSPC | Chol | DSPE- | 113.80 | 0.22 | 96.5 | X |
| | (48.5%) | (10%) | (40%) | PEG2k | | | | |
| | | | | (1.5%) | | | | |
| F-52* | C66 | DSPC | Chol | DSPE- | 86.9 | 0.029 | 99.6 | X |
| | (48.5%) | (10%) | (40%) | PEG2k | | | | |
| | | | | (1.5%) | | | | |
| F-53 | C66 | DSPC | Chol | DMG-PEG2k | 99.95 | 0.057 | 95.8 | X |
| | (48.5%) | (10%) | (41%) | (0.5%) | | | | |
| F-54 | C66 | DSPC | Chol | DMG-PEG2k | 89.13 | 0.082 | 93.9 | X |
| | (48.5%) | (10%) | (40.5%) | (1.0%) | | | | |
| F-55 | C66 | DSPC | Chol | DPPE- | 91.73 | 0.064 | 93.4 | X |
| | (48.5%) | (10%) | (40.5%) | PEG2k | | | | |
| | | | | (1.0%) | | | | |
| F-56 | C66 | DSPC | Chol | DSPE- | 91.10 | 0.064 | 96.4 | X |
| | (48.5%) | (10%) | (40.5%) | PEG2k | | | | |
| | | | | (1.0%) | | | | |
| F-57 | C66 | DSPC | Chol | DPPE- | 89.9 | 0.044 | 92.8 | X |
| | (48.5%) | (10%) | (40%) | PEG2k | | | | |
| | | | | (1.5%) | | | | |
| F-Q1* | S-1 | DSPC | Chol | DPPE- | 80.1 | 0.146 | 97.7 | X |
| | (48.5%) | (10%) | (39.5%) | PEG2k | | | | |
| | | | | (2%) | | | | |
| F-Q2 | C66 | DSPC | Chol | DSPE- | 80 | 0.074 | 97 | X |
| | (48.5%) | (10%) | (39.5%) | PEG2k | | | | |
| | | | | (2%) | | | | |
| F-Q4 | S-1 | DSPC | Chol | DPPE- | 64.3 | 0.095 | 96 | X |
| | (48.5%) | (10%) | (39.5%) | PEG2k | | | | |
| | | | | (2%) | | | | |
| F-Q6 | S-1 | DSPC | Chol | PL-9A | 66.8 | 0.056 | 98.4 | X |
| | (48.5%) | (10%) | (39.5%) | (2%) | | | | |

Buffer X: 25 mM Sodium Acetate, pH 5.0;
Buffer Y: 50 mM Citrate, pH 4.0
a - N:P = 9;
b - N:P = 8;
unless otherwise specified all other formulations N:P = 6
F-52* and FQ1* - Formulated without gRNA as a negative control

### Example 2E: LNP Formulations E—cGFP Formulations

Ionizable lipids, DSPC, cholesterol, and a PEG lipid were dissolved in pure ethanol at the specified mol % ratios with a total lipid concentration of 10.8 mM. A 0.10 mg/mL mRNA solution was prepared using acidic buffer (pH 4.0-5.0) containing mRNA encoding eGFP. The nucleotide and lipid solutions were mixed at a 3:1 volume ratio using the NANOASSEMBLR microfluidic system at a 12 mL/min total flow rate resulting in rapid mixing and self-assembly of LNPs. Formulations were further dialyzed against PBS (pH 7.4) overnight at 4° C., concentrated using centrifugal filtration and filtered (0.2 μm pore size). The particle size and polydispersity index (PDI) of formulations was measured by dynamic light scattering (DLS) using a ZETASIZER ULTRA (Malvern Panalytical). RNA encapsulation efficiency (EE %) was determined by Ribogreen assay.

TABLE 2E
LNP Formulations

| | IL | PL | SL | PEG-L | Size (Z- | | EE | |
|---|---|---|---|---|---|---|---|---|
| Form | (mol %) | (mol %) | (mol %) | (mol %) | avg nm) | PDI | (%) | Buffer |
| F-58 | C66 | DSPC | Chol | DMG-PEG2k | 92.80 | 0.046 | 91 | X |
| | (48.5%) | (10%) | (41%) | (0.5%) | | | | |
| F-59 | C66 | DSPC | Chol | DMG-PEG2k | 94.10 | 0.028 | 80.8 | X |
| | (48.5%) | (10%) | (40.5%) | (1.0%) | | | | |
| F-60 | C66 | DSPC | Chol | DMG-PEG2k | 94.40 | 0.034 | 79 | X |
| | (48.5%) | (10%) | (40%) | (1.5%) | | | | |
| F-61 | C66 | DSPC | Chol | DMG-PEG2k | 93.80 | 0.029 | 72.4 | X |
| | (48.5%) | (10%) | (39.5%) | (2.0%) | | | | |
| F-62 | C66 | DSPC | Chol | DPPE-PEG2k | 91.10 | 0.034 | 94.7 | X |
| | (48.5%) | (10%) | (41%) | (0.5%) | | | | |
| F-63 | C66 | DSPC | Chol | DPPE -PEG2k | 94.70 | 0.034 | 86.1 | X |
| | (48.5%) | (10%) | (40.5%) | (1.0%) | | | | |
| F-64 | C66 | DSPC | Chol | DPPE -PEG2k | 92.60 | 0.026 | 76.2 | X |
| | (48.5%) | (10%) | (40%) | (1.5%) | | | | |
| F-65 | C66 | DSPC | Chol | DPPE -PEG2k | 93.70 | 0.044 | 75.2 | X |
| | (48.5%) | (10%) | (39.5%) | (2.0%) | | | | |
| F-66 | C66 | DSPC | Chol | DSPE-PEG2k | 89.50 | 0.041 | 95.9 | X |
| | (48.5%) | (10%) | (41%) | (0.5%) | | | | |
| F-67 | C66 | DSPC | Chol | DSPE-PEG2k | 88.80 | 0.043 | 81.7 | X |
| | (48.5%) | (10%) | (40.5%) | (1.0%) | | | | |
| F-68 | C66 | DSPC | Chol | DSPE-PEG2k | 90.20 | 0.029 | 77.6 | X |
| | (48.5%) | (10%) | (40%) | (1.5%) | | | | |
| F-69 | C66 | DSPC | Chol | DSPE-PEG2k | 86.90 | 0.029 | 73.3 | X |
| | (48.5%) | (10%) | (39.5%) | (2.0%) | | | | |

Buffer X: 25 mM Sodium Acetate, pH 5.0;
Buffer Y: 50 mM Citrate, pH 4.0
a - N:P = 9;
b - N:P = 8;
unless otherwise specified all other formulations N:P = 6

### Example 2F: LNP Formulation F-Bone Marrow Distribution

Ionizable lipids, phospholipid, cholesterol, PEG-lipid were dissolved in pure ethanol at the specified mol % ratio (Table 2F) with a total lipid concentration of ~10.8 mM. 0.10 mg/mL mRNA solution was prepared using acidic buffer (pH 4.0-5.0) containing mRNAs encoding VHH and Cas9 in a 2:1 ratio. The nucleotide and lipid solutions were mixed at a 3:1 volume ratio using the NANOASSEMBLR microfluidic system at a 135 mL/min total flow rate resulting in rapid mixing and self-assembly of LNPs. Formulations were further dialyzed against PBS (pH 7.4) overnight at 4° C., and buffer exchanged into a sucrose-containing Tris-HCl cryoprotectant buffer for subsequent storage at −80° C. The individual particle sizes of formulations was measured by dynamic light scattering (DLS) using a ZETASIZER ULTRA (Malvern Panalytical). RNA encapsulation efficiency was determined by Ribogreen assay.

TABLE 2F
LNP Formulations

| Form. | IL | PL | PEG | Sterol | N:P/Buffer | Size (nm) | PDI | ee % |
|---|---|---|---|---|---|---|---|---|
| F-Z1 | C66 | DSPC | DSPE-PEG2k | Chol | 6 | 96.45 | 0.07 | 87.6 |
| | 48.5 | 10 | 2 | 39.5 | X | | | |
| F-Z5 | AT-7 | DSPC | DSPE-PEG2k | Chol | 6 | 75.7 | 0.21 | 99.3 |
| | 48.5 | 10 | 2 | 39.5 | X | | | |
| F-Z10 | CX-8 | DSPC | DSPE-PEG2k | Chol | 6 | 96.8 | 0.23 | 95.7 |
| | 48.5 | 10 | 2 | 39.5 | X | | | |
| F-Z11 | S-1 | DSPC | DSPE-PEG2k | Chol | 6 | 79.9 | 0.05 | 92.1 |
| | 48.5 | 10 | 2 | 39.5 | Y | | | |
| F-Z12 | AT-4 | DSPC | DSPE-PEG2k | Chol | 6 | 80.6 | 0.24 | 96.1 |
| | 48.5 | 10 | 2 | 39.5 | X | | | |
| F-Z14 | C66 | DSPC | PL-9A | Chol | 6 | 92.4 | 0.07 | 91.3 |
| | 48.5 | 10 | 2 | 39.5 | X | | | |
| F-Z16 | S-1 | DSPC | PL-9A | Chol | 6 | 80.6 | 0.24 | 96.1 |
| | 48.5 | 10 | 2 | 39.5 | X | | | |
| F-Z17 | AT-4 | DSPC | PL-9A | Chol | 6 | 78.28 | 0.05 | 98.1 |
| | 48.5 | 10 | 2 | 39.5 | X | | | |
| F-Z20 | C66 | DSPC | DPPE-PEG2k | Chol | 6 | 82.3 | 0.12 | 92.0 |
| | 48.5 | 10 | 2 | 39.5 | X | | | |
| F-Z22 | S-1 | DSPC | DPPE-PEG2k | Chol | 6 | 79.5 | 0.02 | 93.5 |
| | 48.5 | 10 | 2 | 39.5 | Y | | | |
| F-Z23 | AT-4 | DSPC | DPPE-PEG2k | Chol | 6 | 82.6 | 0.10 | 97.4 |
| | 48.5 | 10 | 2 | 39.5 | Y | | | |
| F-Z24 | AX-6 | ESM | DMG-PEG2k | Chol | Y | 65.79 | 0.058 | 94.7 |
| | 33 | 40 | 2 | 25 | 8 | | | |
| F-Z25 | AX-6 | ESM + | DMG-PEG2k | Chol | Y | 64.01 | 0.068 | 96.2 |
| | | DSPC | | | | | | |
| | 33 | 20 + 20 | 2 | 25 | 8 | | | |

Buffer X: 25 mM Sodium Acetate, pH 5.0;
Buffer Y: 50 mM Citrate, pH 4.0

### Example 2G: LNP Formulation G-Targeted LNPs

Untargeted LNP were prepared substantially as described in Example 2B. Targeted LNPs were generated using DSPE-PEG2000-mal to modify the LNP using a post-insertion technique, resulting in approximately 0.5% of the LNP content being modified to incorporate the unfunctionalized maleimide PEG lipids (referred to as Mal-LNP). To enable conjugation to maleimide, antibodies were functionalized with SATP (N-succinimidyl-S-acetylthiopropionate). The conjugation took place in phosphate buffered saline (pH 7.4), using 10 equivalents of SATP per antibody. The reaction was allowed to proceed at room temperature for 30 minutes. Deacetylation of SATP was performed using hydroxylamine hydrochloride (NH₂OH·HCl). A 10× stock of NH₂OH·HCl (500 mM, PBS) was prepared, and an appropriate volume was added to the reaction mixture to achieve a final concentration of 1×NH₂OH·HCl. The reaction mixture was shaken for 2 hours at room temperature. Unreacted components were removed at each step using Zeba spin desalting columns (7K MWCO) from THERMOFISHER Scientific. The concentration of the antibody after each step was quantified using absorbance at 280 nm via NANODROP from THERMOFISHER.

The maleimide moieties on the LNP were then conjugated to the reactive sulfhydryl groups on the antibody using thioether conjugation chemistry. The sulfhydryl modified antibody was reacted with 40 equivalents (based on Maleimide content) of Mal-LNP in PBS for 1 hour at room temperature, followed by overnight reaction at 4° C. Excess maleimide on the LNP was capped with 10×N-Acetyl Cysteine (10 mM) for 1 hour at room temperature. The Ab-LNPs were purified using size exclusion chromatography with bench-top qEV Gen2 columns from Izon Sciences, employing PBS as the mobile phase. The first four fractions were collected and pooled together, and then concentrated using 100 KDa MWCO Amicon filters. All targeted and non-targeted LNP preparations were stored at 4° C. and used within three days. The mRNA content was determined using a modified Quant-iT RiboGreen RNA assay from THERMOFISHER Scientific. The size and polydispersity index (PDI) of the modified LNPs were measured using a Malvern Zetasizer.

TABLE 2G
Targeted LNP Formulation

| Form. | IL | PL | Target PEG | PEG | Sterol | Size (nm) | PDI | ee % |
|---|---|---|---|---|---|---|---|---|
| F-76 | C66 | DSPC | — | DSPE- | Chol | 83.84 | 0.033 | 99.3 |
| | | | | PEG2k | | | | |
| | 48.5 | 10 | — | 2 | 39.5 | | | |
| F-77 | C66 | DSPC | DSPE-PEG2k- | DSPE- | Chol | 82.88 | 0.03 | 98.7 |
| | | | Mal-IgG | PEG2k | | | | |
| | 48.5 | 10 | 0.5 | 1.5 | 39.5 | | | |
| F-78 | C66 | DSPC | DSPE-PEG2k- | DSPE- | Chol | 86.29 | 0.07 | 98.4 |
| | | | Mal-ab-1 | PEG2k | | | | |
| | 48.5 | 10 | 0.5 | 1.5 | 39.5 | | | |
| F-79 | C66 | DSPC | DSPE-PEG2k- | DSPE- | Chol | 85.69 | 0.07 | 98.2 |
| | | | Mal-ab-2 | PEG2k | | | | |
| | 48.5 | 10 | 0.5 | 1.5 | 39.5 | | | |
| F-80** | C66 | DSPC | — | DSPE- | Chol | 83.88 | 0.02 | 98.4 |
| | | | | PEG2k | | | | |
| | 48.5 | 10 | — | 2 | 39.5 | | | |

*All formulations made with N:P = 6 in Buffer X: 25 mM Sodium Acetate and VHH payload, as described in Example 2B, unless otherwise indicated.
**Formulated with fLuc mRNA payload DSPE-PEG2k-Mal (Capped) = DSPE-PEG2K malemide,
with all of the malemide functionalities capped with N-acetyl cysteine
DSPE-PEG2k-Mal-IgG = DSPE-PEG2K malemide functionalized with immunoglobulin G
DSPE-PEG2k-Mal-ab-1 = DSPE-PEG2K malemide functionalized with full length monoclonal Rat IgG2k clone A3C6E2 antibody
DSPE-PEG2k-Mal-ab-2 = DSPE-PEG2K malemide functionalized with full length monoclonal Rat IgG2k clone 104D2 antibody

### Example 3: LNP Delivery to HSCs-Ai14 Mice

LNP/mRNA formulations were prepared as described in Example 2A. Ai14 mice (B6.Cg-Gt (ROSA) 26Sortm14 (CAG-tdTomato) Hze/J (JACKSON LABORATORY 007914)), were injected via tail vein with 0.5 or 1.0 mg/kg cre mRNA (TRILINK BIOTECHNOLOGIES) formulated in LNP formulations F-1 through F-24 in a total volume of 5 mL/kg. Each formulation was dosed in 2-3 mice and an additional 1-2 mice were dosed with either PBS or fLuc mRNA LNP to serve as a control. 72 h post injection, animals were euthanized by CO₂ inhalation, and spleens, femurs, and tibiae/fibulae were harvested. Harvested spleens were dissociated into single cell suspension of splenocytes using the gentleMACS Octo Dissociator with Heaters (MILTENYI 130-096-427) with the Mouse Spleen Dissociation Kit (MILTENYI 130-095-926) per manufacturer's instructions. Dissociated splenocytes were then passed through a 70 μm filter (MILTENYI 130-098-462) and washed with 1×PBS (THERMOFISHER 10010049) containing 2 mM EDTA (THERMOFISHER 15575-020) and 0.5% BSA (MILTENYI 130-091-376). Red blood cells were lysed using ACK Lysing Buffer (THERMOFISHER A1049201) and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA and counted (VICELL XR, BECKMAN COULTER 731196). Cells were diluted, plated (5,000,000 per well) in a 96-well round bottom plate (COSTAR 3799), and stained for flow cytometry. Bone marrow (BM) was harvested from the bones by using a 25 G needle (BD 305122) to flush the marrow cavity with 1×PBS+2 mM EDTA+0.5% BSA. Cells were collected, passed through a 70 μm filter, and washed with 1×PBS+2 mM EDTA+0.5% BSA. Red blood cells were lysed using ACK Lysing Buffer and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA and counted. Cells were diluted, plated (~10 million per well) in a 96-well bottom plate, and stained for flow cytometry. Briefly, cells were stained in 1×PBS with Live/Dead Fixable Aqua (INVITROGEN L34966) at 1:1000 for 20 min at room temperature. Cells were then washed twice with Cell Staining Buffer (BIOLEGEND 420201) and incubated with Fc block (splenocytes) or labeled CD16/32 antibody (bone marrow) for 5 min at 4° C. and surface antibody stains either in full or FMO master mixes (panel and dilutions shown below in Table 3A) added on top of the Fc Block for an additional 30 min at 4° C. Cells were then washed three times with Cell Staining Buffer and fixed with CYTOFIX (BD 554655) at 4° C. for 30 min. Cells were washed twice with 1×PBS and filtered through a 30-40 μm filter (Pall 8027) and acquired on cytometer (THERMOFISHER Attune NXT with a laser configuration of Blue (3)/Red (3)/Violet (4)/Yellow (4)) equipped with a high-throughput autosampler (THERMOFISHER CYTKICK). Compensation was performed using ULTRACOMP eBeads (THERMOFISHER 01-3333-41), ArC Amine Reactive Compensation Bead Kit (THERMOFISHER A10346), and Luciferase/tdTomato Dual-Reporter HEK293 cells (Alstem LRL01). Analysis performed using FLOWJO software (BD V10.8.1). Cells were identified with markers in Tables 3C and 3D and tdTomato gates placed so that the negative control would be ≤0.5%⁺.

The percentage of cells of the noted varieties expressing cre mRNA is reported below in Table 3E. Several LNP formulations demonstrated high delivery (>40%) to HSPCs with minimal off-target expression. Specifically, high delivery by many of the LNP formulations to LT-HSCs strongly suggests that delivery of gene editing systems in vivo can be used to treat hemoglobinopathies in a subject in need thereof.

TABLE 3A
Splenocyte surface antibody stains used in Example 3

| | | | | | | Usage |
|---|---|---|---|---|---|---|
| | | | | | | per test |
| Staining | | | | | | (dilution; |
| Step | Target | Fluor | Clone | Source | Cat No. | 1:X) |
| Live/Dead | Viability | Aqua | N/A | INVITROGEN | L34966 | 1000 |
| Surface | Super Bright Staining Buffer | | | eBioscience | SB-4401-75 | 20 |
| | Fc Block | N/A | S17011E | BIOLEGEND | 156604 | 200 |
| | CD49b | AF488 | DX5 | BIOLEGEND | 108913 | 200 |
| | CD11b | PerCP/Cy5.5 | M1/70 | BIOLEGEND | 101228 | 200 |
| | F4/80 | APC | BM8 | BIOLEGEND | 123116 | 200 |
| | NKp46 | AF700 | 29A1.4 | BD | 561169 | 500 |
| | CD11c | APC/Fire750 | N418 | BIOLEGEND | 117352 | 200 |
| | CD19 | BV421 | 6D5 | BIOLEGEND | 115538 | 400 |
| | IA/IE | BV605 | M5/114.15.2 | BIOLEGEND | 107639 | 500 |
| | CD8a | BV711 | 53-6.7 | BIOLEGEND | 100748 | 200 |
| | TCRb | PE/Cy7 | H57-597 | BIOLEGEND | 109222 | 200 |
| Reporter | tdTomato | tdTomato | N/A | N/A | N/A | N/A |
| (not stained) | | | | | | |

For clarity, in later panels NKp46 was replaced with AF700 NK-1.1 clone S17016D used at a 1:500 dilution (BIOLEGEND 156512)

TABLE 3B
Bone marrow surface antibody stains used in Example 3

| | | | | | | Usage |
|---|---|---|---|---|---|---|
| | | | | | | per test |
| Staining | | | | | | (dilution; |
| Step | Target | Fluor | Clone | Source | Cat No. | 1:X) |
| Live/Dead | Viability | Aqua | N/A | INVITROGEN | L34966 | 1000 |
| Surface | Super Bright Staining Buffer | | | eBioscience | SB-4401-75 | 20 |
| | CD16/32 | AF700 | S17011E | BIOLEGEND | 156620 | 200 |
| | cKit | AF488 | 2B8 | BIOLEGEND | 105816 | 200 |
| | CD135 | PerCP-eFluor710 | A2F10 | Thermo | 46-1351-82 | 100 |
| | CD34 | AF647 | RAM34 | BD | 560230 | 100 |
| | CD48 | APC/Fire750 | HM48-1 | BIOLEGEND | 103446 | 200 |
| | Sca1 | BV421 | D7 | BIOLEGEND | 108128 | 200 |
| | CD127 | BV605 | A7R34 | BIOLEGEND | 135041 | 100 |
| | CD150 | BV711 | TC15-12F12.2 | BIOLEGEND | 115941 | 200 |
| | CD4 | PE/Cy7 | GK1.5 | BIOLEGEND | 100422 | 200 |
| | CD8a | PE/Cy7 | 53-6.7 | BIOLEGEND | 100722 | 200 |
| | CD11b | PE/Cy7 | M1/70 | BIOLEGEND | 101216 | 200 |
| | B220 | PE/Cy7 | RA3-6B2 | BIOLEGEND | 103222 | 200 |
| | Gr1 | PE/Cy7 | RB6-8C5 | BIOLEGEND | 108416 | 200 |
| | Ter119 | PE/Cy7 | TER119 | BIOLEGEND | 116222 | 200 |
| Reporter | tdTomato | tdTomato | N/A | N/A | N/A | N/A |
| (not stained) | | | | | | |

TABLE 3C
Definitions of Cell Subsets in Mouse Spleen
by Flow Cytometry used in Example 3

| Cell Type: | Surface Marker Phenotype: |
|---|---|
| B cells | L/DAqua−TCRb−CD19+ |
| T cells | L/DAqua−CD19−TCRb3+ |
| CD4 T cells | L/DAqua−CD19−TCRb+CD8− |
| CD8 T cells | L/DAqua−CD19−TCRb+CD8+ |
| NK cells* | L/DAqua−CD19−TCRb−F4/80−CD11b+CD49b+ |
| Red Pulp Macrophages | L/DAqua−CD19−TCRb−F4/80^{hi}CD11b^{lo} |
| Dendritic Cells | L/DAqua−CD19−TCRb−F4/80−IAIE+CD11c+ |

TABLE 3D
Definition of Cell Subsets in Mouse BM
by Flow Cytometry used in Example 3

| Cell Type: | Surface Marker Phenotype: |
|---|---|
| CLP | L/DAqua−Lineage−Sca1+cKit^{lo}CD135+CD127+ |
| CMP | L/DAqua−Lineage−Sca1−cKit+CD16/32^{mid}CD34+ |
| GMP | L/DAqua−Lineage−Sca1−cKit+CD16/32^{hi}CD34+ |
| MEP | L/DAqua−Lineage−Sca1−cKit+CD16/32−CD34− |
| LSK | L/DAqua−Lineage−Sca1+cKit+ |
| LT-HSC | L/DAqua−Lineage−Sca1+cKit+CD34−CD135− |
| ST-HSC | L/DAqua−Lineage−Sca1+cKit+CD34+CD135− |
| MPP | L/DAqua−Lineage−Sca1+cKit+CD34+CD135+ |
| LT-HSC | L/DAqua−Lineage−Sca1+cKit+CD150+CD48− |
| (alternative)‡ | |
| ST-HSC | L/DAqua−Lineage−Sca1+cKit+CD150−CD48− |
| (alternative) | |
| MPP | L/DAqua−Lineage−Sca1+cKit+CD150+CD48+ |
| (alternative) | |

*Lineage is defined as including CD4, CD8a, CD11b, B220, GR1, Ter119.
‡HSCs gated two different ways, alternative denotes second strategy for gating specific HSC population.

TABLE 3E
Percentage of cells of various types expressing cre mRNA

| | | HSPCs (%) | | | Spleen (%) | | | | | | |
|---|---|---|---|---|---|---|---|---|---|---|---|
| | Dose | LT- | ST- | | | | | RP | Lung (%) | | |
| Form | (mg/kg) | HSC | HSC | MPP | T | NK | DC | mac | T | DC | EC |
| F-1 | 1.0 | 28.3 | — | — | 1 | 5.6 | 24.3 | 68.3 | 0.34 | 3.2 | 11.2 |
| F-2 | 1.0 | 40.3 | 50.0 | 65.9 | 2.2 | 8.6 | 30.2 | 83.2 | 1.7 | 9.3 | 17.1 |
| F-3 | 1.0 | 48.2 | — | — | 1 | 4.6 | 33.4 | 95.5 | 0.75 | 3.8 | 19.7 |
| F-4 | 1.0 | 39.6 | 47.5 | 49.9 | 2.5 | 6.6 | 28.9 | 94.4 | 1.5 | 4.2 | 19.9 |
| F-5 | 1.0 | 21.4 | — | — | 0.87 | 3.9 | 18.5 | 81.8 | 0.3 | 1.8 | 10.1 |
| F-6 | 1.0 | 20.1 | 24.2 | 24.6 | 1.85 | 4.2 | 16.1 | 89.0 | 0.5 | 1.7 | 3.0 |
| F-7 | 1.0 | 66 | 82 | 73.4 | 1.4 | 4.5 | 12.1 | 98.2 | 1 | 5.6 | 5.1 |
| F-8 | 1.0 | 61 | 82 | 57.1 | 1.4 | 4.2 | 11.9 | 99.4 | 1 | 4.5 | 3.7 |
| F-9 | 1.0 | 37.8 | 62.8 | 32.7 | 1.2 | 2.9 | 7.7 | 98.1 | 0.3 | 0.8 | 0.61 |
| F-10 | 0.5 | 33.8 | 32.8 | 17.9 | 3 | 8.4 | 28.5 | 96.6 | 3.5 | 1.3 | 24.7 |
| F-11 | 0.5 | 33.5 | 28 | 18.8 | 3 | 8.5 | 26.4 | 96.1 | 1.4 | 1.0 | 5.2 |
| F-12 | 0.5 | 33.2 | 28.3 | 17.2 | 2 | 9.8 | 38.0 | 92.8 | 1.0 | 1.5 | 5.4 |
| F-13 | 0.5 | 53.8 | 54 | 51.5 | 3.9 | 4.7 | 12.0 | 96.9 | 1.3 | 13.5 | 8.2 |
| F-14 | 0.5 | 45.6 | 44.5 | 43.7 | 5.5 | 6.3 | 19.1 | 97.0 | 2.5 | 23.5 | 18.5 |
| F-15 | 0.5 | 44.3 | 42.7 | 41.1 | 3.2 | 5.5 | 12.7 | 96 | 0.7 | 2.3 | 4.4 |
| F-16 | 0.5 | 46.7 | 38.3 | 20.5 | 2.4 | 5.5 | 24 | 95.7 | 1.63 | 15.1 | 7.42 |
| F-17 | 0.5 | 44.2 | 40 | 31.2 | 2.3 | 4.2 | 9.7 | 96 | 0.73 | 7.6 | 7.7 |
| F-18 | 0.5 | 50 | 44.5 | 27.7 | 2.2 | 3.4 | 7.9 | 95 | 1.4 | 10.2 | 8.6 |
| F-19 | 0.5 | 63.8 | 50.2 | 29.5 | 3.8 | 6.6 | 49 | 97.7 | 4.09 | 26.3 | 11.8 |
| F-20 | 0.5 | 41.6 | 57.5 | 22.6 | 2.1 | 3.5 | 41 | 90.8 | 1.3 | 10.3 | 3.39 |
| F-21 | 0.5 | 52.8 | 80.7 | 51.35 | 2.2 | 2.5 | 14.25 | 92.7 | 2.2 | 11.3 | 4.53 |
| F-22 | 0.5 | 73.5 | 88.8 | 64.6 | 2.75 | 17 | 19.9 | 92.2 | 1.45 | 10.2 | 7.7 |
| F-23 | 0.5 | 31 | 54.1 | 37.5 | 1.82 | 2.2 | 6.5 | 95.2 | 0.97 | 6.17 | 2.05 |

T—T Cells;
NK—Natural Killer Cells;
RP Mac—red pulp macrophages;
DC—dendritic cells;
EC—endothelial cells

TABLE 3F
Percentage of cells of various types expressing cre mRNA

| | Dose | Bulk | LT-HSC | ST-HSC | MPP |
|---|---|---|---|---|---|
| Form | (mg/kg) | LSK (%) | (%) | (%) | (%) |
| F-27 | 0.5 | 55.32 | 46.93 | 53.30 | 56.30 |
| F-28 | 0.5 | 47.8 | 48.0 | 54.2 | 45.93 |
| F-29 | 0.5 | 22.4 | 22.7 | 27.13 | 23.9 |
| F-30 | 0.5 | 39.33 | 40.03 | 44.27 | 38.37 |
| F-31 | 0.5 | 46.7 | 44.83 | 52.33 | 47.17 |
| F-32 | 0.5 | 7.61 | 7.14 | 11.78 | 7.58 |
| F-33 | 0.5 | 32.1 | 29.1 | 48.23 | 28.2 |
| F-34 | 0.5 | 40.07 | 46.7 | 52.33 | 29.83 |
| F-35 | 0.5 | 75.3 | 39.2 | 45.3 | 61 |
| F-36 | 0.5 | 88.5 | 68.93 | 71.5 | 82.8 |
| F-37 | 0.5 | 60.53 | 20.4 | 31.23 | 44.5 |
| F-38 | 0.5 | 84.2 | 56.43 | 56.9 | 76.73 |
| F-39 | 0.5 | 30.93 | 11.24 | 12.6 | 22.33 |
| F-40 | 0.5 | 82.23 | 52.5 | 55.6 | 73.3 |
| F-41 | 0.5 | 18.9 | 18.4 | 23.1 | 15.7 |
| F-42 | 0.5 | 35.3 | 32 | 39.6 | 32.4 |
| F-43 | 0.5 | 12 | 13.4 | 14.83 | 9.8 |
| F-44 | 0.5 | 20.5 | 20.84 | 24 | 17.33 |

### Example 4: LNP Delivery to HSCs-Non-Human Primates

LNP formulations were prepared as described in Example 2B. Each formulation was administered to 3 Cynomolgus monkeys on Day 1 via 60-min intravenous infusion into an appropriate peripheral vein using an infusion pump at a dose level of 2.0 mg/kg mRNA (dose volume of 5 mL/kg; concentration 0.4 mg/mL). Blood samples were collected at 24 hours after dosing for peripheral blood mononuclear cells, and at 24 hours the test subjects were terminated. Test subject femurs were flushed for collection of bone marrow and spleens were collected for analysis by flow cytometry. Up to six grams of harvested spleen per animal was dissociated into single cell suspension of splenocytes using the gentleMACS Octo Dissociator with Heaters (MILTENYI 130-096-427) with the Multi Tissue Dissociation Kit I (MILTENYI 130-110-201) per manufacturer's instructions. Dissociated splenocytes were then passed through a 70 μm filter (MILTENYI 130-098-462) and washed with 1×PBS (THERMOFISHER 10010049) containing 2 mM EDTA (THERMOFISHER 15575-020) and 0.5% BSA (MILTENYI 130-091-376). Red blood cells were lysed using ACK Lysing Buffer (THERMOFISHER A1049201) and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA and counted (VICELL XR, BECKMAN COULTER 731196). Cells were diluted, plated (5,000,000 per well) in a 96-well round bottom plate (COSTAR 3799), and stained for flow cytometry. Flushed bone marrow was centrifuged and cells were collected, passed through a 70 μm filter, and washed with 1×PBS+2 mM EDTA+0.5% BSA. Red blood cells were lysed using ACK Lysing Buffer and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA and counted. Cells were diluted, plated (10 million per well) in a 96-well bottom plate, and stained for flow cytometry. Briefly, cells were stained in 1×PBS with Live/Dead Fixable Aqua (INVITROGEN L34966) at 1:1000 for 20 min at room temperature. Cells were then washed twice with Cell Staining Buffer (BIOLEGEND 420201) and incubated with Fc block for 5 min at 4° C. and surface antibody stains either in full or FMO (fluorescence minus one) master mixes (panel and dilutions shown below in Table 4A) added on top of the Fc Block for an additional 30 min at 4° C. Cells were then washed three times with Cell Staining Buffer and fixed with CYTOFIX (BD 554655) at 4° C. for 30 min. Cells were washed twice with 1×PBS and filtered through a 30-40 μm filter (Pall 8027) and acquired on cytometer (THERMOFISHER Attune NXT with a laser configuration of Blue (3)/Red (3)/Violet (4)/Yellow (4)) equipped with a high-throughput autosampler (THERMOFISHER CytKick). Compensation was performed using UltraComp eBeads (THERMOFISHER 01-3333-41) and ArC Amine Reactive Compensation Bead Kit (THERMOFISHER A10346). Analysis performed using FLOWJO software (BD V10.8.1). Cells were identified with markers in Tables 4C and 4D and VHH reporter+ gates placed so that the negative control would be ≤0.5%⁺.

The percentage of cells of the noted varieties expressing VHH mRNA is reported below in Table 4E. Robust VIIII expression was detected in many organs, including bone marrow HSPCs. Formulation F-24 performed especially well for delivery to LT-HSCs.

TABLE 4A
Splenocyte surface antibody stains used in Example 4

| | | | | | | Usage |
|---|---|---|---|---|---|---|
| Staining | | | | | | per test |
| Step: | Target | Fluorophore | Clone | Manufacturer | Cat No. | (dilution) |
| Live/Dead | Viability | Aqua | N/A | INVITROGEN | L34966 | 1000 |
| Surface | Super Bright Staining Buffer | | | EBIOSCIENCE | SB-4401-75 | 20 |
| | VHH | iFluor 488 | 96A3F5 | GENSCRIPT | A01862 | 100 |
| | CD159a | APC | Z199 | BECKMAN COULTER | A60797 | 10 |
| | CD4 | AF700 | OKT4 | BIOLEGEND | 317426 | 20 |
| | CD20 | APC/Fire 750 | 2H7 | BIOLEGEND | 302358 | 20 |
| | HLA-DR | BV605 | G46-6 | BD | 562845 | 100 |
| | CD8 | BV711 | SK1 | BIOLEGEND | 344734 | 20 |
| | CD11c | PE | 3.9 | BIOLEGEND | 301606 | 20 |
| | CD3 | PE-CF594 | SP34-2 | BD | 562406 | 20 |
| | CD163 | PE-Cy7 | GHI/61 | BIOLEGEND | 326514 | 20 |

TABLE 4B
NHP Bone Marrow antibodies used in Example 4

| | | | | | | Usage |
|---|---|---|---|---|---|---|
| Staining | | | | | | per test |
| Step: | Target | Fluorophore | Clone | Manufacturer | Cat No. | (dilution) |
| Live/Dead | Viability | Aqua | N/A | INVITROGEN | L34966 | 1000 |
| Surface | Super Bright Staining Buffer | | | EBIOSCIENCE | SB-4401-75 | 20 |
| | VHH | iFluor 488 | 96A3F5 | GENSCRIPT | A01862 | 100 |
| | CD38 | APC | OKT10 | CAPRICO | 100845 | 50 |
| | CD3 | AF700 | SP34-2 | BD | 557917 | 20 |
| | CD20 | AF700 | 2H7 | BIOLEGEND | 302322 | 20 |
| | CD11c | AF700 | 3.9 | BIOLEGEND | 301648 | 20 |
| | CD14 | AF700 | M5E2 | BIOLEGEND | 301822 | 50 |
| | CD8 | AF700 | SK1 | BIOLEGEND | 344724 | 20 |
| | CD34 | BV421 | 561 | BIOLEGEND | 343610 | 20 |
| | CD45RA | BV605 | 5H9 | BD | 740424 | 20 |
| | CD49f | PE | GoH3 | BIOLEGEND | 313612 | 20 |
| | CD90 | PE-Cy7 | 5E10 | BIOLEGEND | 328124 | 20 |

TABLE 4C
Definition of Cell Subsets in NHP Spleen
by Flow Cytometry used in Example 4

| Cell Type: | Surface Marker Phenotype: |
|---|---|
| B cells | L/DAqua−CD20+CD3− |
| T cells | L/DAqua−CD20−CD3+ |
| CD4 T cells | L/DAqua−CD20−CD3+CD4+CD8− |
| CD8 T cells | L/DAqua−CD20−CD3+CD4−CD8+ |
| NK cells | L/DAqua−CD20−CD3−HLADR−CD159a+ |
| Red Pulp Macrophages | L/DAqua−CD20−CD3−HLADR+CD163+ |
| Dendritic Cells | L/DAqua−CD20−CD3−HLADR+CD11c+ |

TABLE 4D
Definition of Cell Subsets in NHP BM
by Flow Cytometry used in Example 4

| Cell Type: | Surface Marker Phenotype: |
|---|---|
| MLP | L/DAqua−Lineage−CD34+CD45RA+CD38−CD90− |
| CMP | L/DAqua−Lineage−CD34+CD45RA−CD38^{hi} |
| GMP | L/DAqua−Lineage−CD34+CD45RA+CD38+ |
| MEP | L/DAqua−Lineage−CD34+CD45RA+CD38^{mid} |
| HSC | L/DAqua−Lineage−CD34+CD45RA−CD38−CD90+CD49f+ |
| MPP | L/DAqua−Lineage−CD34+CD45RA−CD38−CD90−CD49f− |

*Lineage is defined as including CD3, CD8, CD14, CD20, and CD11c.

TABLE 4E
Percentage of cells of various types expressing VHH mRNA

| | | HSPCs (%) | | | Spleen (%) | | | | | | |
|---|---|---|---|---|---|---|---|---|---|---|---|
| | Dose | LT- | ST- | | | | | RP | Lung (%) | | |
| Form | (mg/kg) | HSC | HSC | MPP | T | NK | DC | mac | T | DC | EC |
| F-24 | 2.0 | 26.7 | 32 | 5.4 | 9.7 | 29 | 83.9 | 89.7 | 1.5 | 70 | 5.25 |
| F-25 | 2.0 | 8.15 | 13 | 1.9 | 4.6 | 29 | 84.9 | 94 | 1.81 | 39.8 | 1.57 |
| F-26 | 2.0 | 2.27 | 11.6 | 0.14 | 2.22 | 30 | 65 | 78.6 | 0.41 | 4.34 | 0.42 |

TABLE 4F
Percentage of cells of various types expressing VHH mRNA

| | Dose | Bone Marrow (%) | | | Lung (%) | | | | |
|---|---|---|---|---|---|---|---|---|---|
| Form | (mg/kg) | Bulk CD34+ | All HSC | MPP | B | T | DC | EC | Alv Macs |
| F-72 | 2.0 | 10.69 | 6.22 | 6.43 | 1.33 | 0.98 | 75.1 | 0.93 | 7.04 |
| F-73 | 2.0 | 6.86 | 7.20 | 5.36 | 10.89 | 1.09 | 65.0 | 1.92 | 3.01 |
| F-74 | 2.0 | 7.16 | 8.13 | 3.58 | 2.16 | 0.94 | 58.5 | 0.92 | 6.17 |
| F-75 | 2.0 | 22.93 | 12.83 | 26.5 | 5.69 | 6.17 | 83.8 | 3.16 | 8.38 |

| | Dose | Spleen (%) | | | | | | |
|---|---|---|---|---|---|---|---|---|
| Form | (mg/kg) | B | T | CD4+ T | CD8+ T | NK | RP mac | DC |
| F-72 | 2.0 | 12.6 | 7.6 | 6.7 | 7.2 | 38.3 | 73.4 | 26.1 |
| F-73 | 2.0 | 36.0 | 10.0 | 7.9 | 11.2 | 48.6 | 87.4 | 53.3 |
| F-74 | 2.0 | 29.6 | 10.2 | 6.5 | 10.7 | 54.0 | 63.3 | 47.1 |
| F-75 | 2.0 | 15.6 | 10.7 | 10.1 | 10.2 | 43.2 | 69.2 | 36.3 |

*Alv Macs = Alveolar Macrophages

### Example 4B: Colony Forming Assay for CD34+ Lineage− Bone Marrow Cells from NHP

LNP formulations were prepared as described in Example 2B. Each formulation was administered to 3 Cynomolgus monkeys on Day 1 via 60-min intravenous infusion into an appropriate peripheral vein using an infusion pump at a dose level of 2 mg/kg mRNA (dose volume of 5 mL/kg; concentration 0.4 mg/mL). Bone marrow aspirate samples were collected 24 hours after dosing. Bone marrow aspirate was centrifuged, and red blood cells lysed using ACK Lysing Buffer, the remaining cells were then washed and stained with surface antibodies in full master mix (panels and dilutions shown below in Tables 4G-4I). Cells were then washed and acquired on SORTER (SONY MA900). Single stain controls were made using UltraComp eBeads (THERMOFISHER 01-3333-41) and ArC Amine Reactive Compensation Bead Kit (THERMOFISHER A10346). Cells were sorted into VHH+ and VHH-fractions as defined in Table 4H. Fractions were resuspended in METHOCULT™ H4434 Classic (STEMCELL 04464) at concentrations of 1000 cells/ml and plated in SMARTDISH™ (STEMCELL 273171). Wells were imaged on D12 using the STEMVISION™ plate reader (SEMCELL 22007). Analysis was performed using COLONY MARKER analysis software. Both VHH+ and VHH-CD34+ cells showed colony formation indicating that CD34+Lin− cells that were delivered to by the LNPs maintained the stem cell potential.

TABLE 4G
NHP Bone Marrow antibodies used in Example 4B

| | | | | | | Usage per |
|---|---|---|---|---|---|---|
| Staining | | | | | | test |
| Step: | Target | Fluorophore | Clone | Manufacturer | Cat No. | (dilution) |
| Live/Dead | Viability | Aqua | N/A | INVITROGEN | L34966 | 1000 |
| Surface | Super Bright Staining Buffer | | | EBIOSCIENCE | SB-4401-75 | 20 |
| | VHH | iFluor 488 | 96A3F5 | GENSCRIPT | A01862 | 100 |
| | CD3 | AF700 | SP34-2 | BD | 557917 | 20 |
| | CD20 | AF700 | 2H7 | BIOLEGEND | 302322 | 20 |
| | CD11b | AF700 | ICRF44 | BIOLEGEND | 301356 | 20 |
| | CD11c | AF700 | 3.9 | BIOLEGEND | 301648 | 20 |
| | CD14 | AF700 | M5E2 | BIOLEGEND | 301822 | 20 |
| | CD16 | AF700 | 3G8 | BIOLEGEND | 302026 | 20 |
| | CD4 | AF700 | OKT4 | BIOLEGEND | 317426 | 20 |
| | CD8 | AF700 | SK1 | BIOLEGEND | 344724 | 20 |
| | CD34 | BV421 | 561 | BIOLEGEND | 343610 | 20 |

TABLE 4H
Definition of Cell Subsets in NHP BM aspirate
by Flow Cytometry used in Example 4B

| Cell Type: | Surface Marker Phenotype: |
|---|---|
| VHH+ | L/DAqua−CD34+Lineage−VHH+ |
| VHH− | L/DAqua−CD34+Lineage−VHH− |

*Lineage is defined as including CD3, CD4, CD8, CD14, CD16, CD20, CD11b, and CD11c.

TABLE 4I
Average colony counts at day 12 CFU plated samples
Average number of colonies from three animals in duplicated wells.

| Formulation | VHH− | VHH+ |
|---|---|---|
| F-85 | 115 | 136 |
| F-86 | 80 | 159 |

### Example 5: In Vivo Editing Delivery in Humanized Mice

### In Vivo Protocol

Humanized mice, ranging from 6-10 weeks of age are used in each study. LNPs prepared as described in Example 2C are dosed via the lateral tail vein in a volume of approximately 5 mL per kilogram (mpk) body weight. The animals are periodically observed for adverse effects throughout the study. Mice are dosed at about 0.2-0.5 mpk. Animals are euthanized at 7 days by exsanguination via cardiac puncture under isoflurane anesthesia. Tissue samples, including bone marrow from at least one major bone (femur, pelvis, sternum), are collected from each animal for DNA extraction and analysis. Blood is collected into serum separator tubes or into tubes containing buffered sodium citrate for plasma. Cohorts of mice are measured for gene editing by Next-Generation Sequencing (NGS). Overall health and wellbeing of animals is recorded to determine whether delivery of gene editing payloads resulted in deleterious off-target editing.

### NGS Sequencing

In brief, to quantitatively determine the efficiency of editing at the target location in the genome, genomic DNA is isolated and deep sequencing is utilized to identify the presence of insertions and deletions (“indels”) introduced by gene editing. PCR primers are designed around the target site (e.g., BCL11A), and the genomic area of interest is amplified. Additional PCR is performed according to the manufacturer's protocols (e.g., ILLUMINA) to add the necessary chemistry for sequencing. The amplicons are sequenced on a sequencer (e.g., ILLUMINA NEXTSEQ 2000 instrument). The reads are aligned to the relevant reference genome after eliminating those having low quality scores. The resulting files containing the reads are mapped to the reference genome (BAM files), where reads that overlap the target region of interest are selected and the number of wild type reads versus the number of reads which contain an insertion, substitution, or deletion are calculated. The editing percentage (e.g., the “editing efficiency” or “percent editing”) is defined as the total number of sequence reads with insertions or deletions over the total number of sequence reads, including wild type.

### Example 6: LNP Delivery of VHH mRNA to HSCs-BALB/c Mice

LNP formulations were prepared as described in Example 2B. Each formulation was administered to 3 BALB/c mice on Day 1 injected via tail vein with 2.0 mg/kg VHH mRNA formulated in LNP formulations F-45 through F-51 in a total volume of 5 mL/kg. Each formulation was dosed in 2-3 mice and an additional 1-2 mice were dosed with fLuc mRNA LNP to serve as a control. 24 h post injection, animals were euthanized by CO₂ inhalation, and spleens, femurs, and tibiae/fibulae were harvested. Organs were processed as described in Example 3. Cells were prepared, plated and stained as described in Example 3.

Flow cytometry was used to define long-term hematopoietic stem cells (LT-HSC) based on the following: Viable, lineage negative, Sca-1+, c-Kit+, CD150+, CD48−.

The percentage of cells of the noted varieties expressing VHH is reported below in Table 6A. Several LNP formulations demonstrated high delivery (>40%) to HSPCs with minimal off-target expression. Specifically, high delivery by many of the LNP formulations to LT-HSCs strongly suggests that delivery of gene editing systems in vivo can be used to treat hemoglobinopathies in a subject in need thereof.

TABLE 6A
Percentage of cells of various types expressing VHH mRNA

| Formulation | Bulk LSK (%) | LT-HSC (%) |
|---|---|---|
| F-24 | 9.4 | 32.6 |
| F-45 | 9.2 | 31.9 |
| F-46 | 10.2 | 35.9 |
| F-47 | 12.5 | 44.6 |
| F-48 | 25.9 | 61.2 |
| F-49 | 24.3 | 53.0 |
| F-50 | 25.0 | 53.6 |
| F-51 | 9.9 | 31.7 |

In certain embodiments, the protocol of Example 6 is translatable to additional animal models, including but not limited to humanized strains. In certain embodiments, in vivo LNP delivery of VHH mRNA is tested according to the above protocol, or one substantially similar as would be appreciated by a person of ordinary skill in the art, in huCD34+ NSG mice.

### Example 7: Adoptive Transfer of Bone Marrow after In Vivo LNP Delivery of Cre mRNA

### Primary Bone Marrow Transplantation

Female Ai14 mice (B6.Cg-Gt (ROSA) 26Sortm14 (CAG-tdTomato) Hze/J (Jackson Laboratory 007914) CD45.2 background) are dosed with an LNP formulation prepared as described in Example 2A at a dosage of 0.5 mg/kg i.v. (eg. with Formulation F-7 or similar), serving as primary donors. At 2 weeks post dose, Ai14 donors are euthanized, and the femurs and tibiae are harvested, flushed out with RPMI (Gibco 72400-047) containing 0.5% BSA (MILTENYI 130-091-376), filtered through a 70 μm cell strainer (MILTENYI 130-098-462) and then centrifuged (500 g, 3 min). The resulting cell pellet is subjected to RBC lysis using ACK buffer (Thermo Fisher A1049201) for 5 min, diluted with RPMI containing 0.5% BSA (cRPMI), filtered, centrifuged, and resuspended in cRPMI and counted. A fraction of BM cells are stained for flow cytometry (see Flow Cytometry section below) to determine the frequency of tdTomato in donor cell fractions. The remaining bone marrow cells are washed with PBS, re-counted, and resuspended in sterile PBS (for example, at 5×10⁶/200 μl PBS, 5×10⁶ total BM cells) and are adoptively transferred into immunodeficient NBSGW primary recipient mice (NOD.Cg-Kit^{W-41J}Tyr⁺Prkdc^{scid}II2rg^{tm1Wj}1/ThomJ, CD45.1 background). Primary recipient mice are survival bled bi-weekly/monthly for up to 12-16 weeks and analyzed for myeloid and lymphoid cell chimerism and frequency of tdTomato by flow cytometry. Leukocytes are also collected for analysis of tdTomato frequency in immune cells (B cells, T cells, NK cells, neutrophils, etc.)

### Secondary Bone Marrow Transplantation

For a serial secondary bone marrow transplantation, a cohort of the primary recipient mice from the first bone marrow transplantation are euthanized and serve as donors for a pool of secondary recipient mice. Bone marrow from the primary recipient mice is harvested as described above and depleted of lineage-positive cells by magnetic columns (Stem cell technologies) to enrich for HSPCs. Enriched cells are stained for flow cytometry to determine the frequency of tdTomato in the donor cell fraction, and the remaining cells from each donor are transplanted to secondary recipient NBSGW mice (cell numbers ranging from 300,000-800,000 in 200 μl of PBS). Secondary recipient mice are survival bled bi-weekly/monthly for up to 16 weeks and analyzed for myeloid and lymphoid cell chimerism and frequency for tdTomato by flow cytometry.

### Flow Cytometry Staining

To determine the frequency of tdTomato in the donor bone marrow cells, ~10 million cells per well are plated in a 96-well bottom plate, stained in 1×PBS with Live/Dead Fixable Aqua (INVITROGEN L34966) at 1:1000 for 20 min at room temperature. Cells are then washed twice with Cell Staining Buffer (BIOLEGEND 420201) and incubated with labeled CD16/32 antibody (bone marrow) for 5 min at 4° C. and surface antibody stains is added on top of the Fc Block for an additional 30 min at 4° C. Cells are then washed three times with Cell Staining Buffer and fixed with CYTOFIX (BD 554655) at 4° C. for 30 min. Cells are washed twice with 1×PBS and filtered through a 30-40 μm filter (Pall 8027) and acquired on a cytometer (THERMOFISHER Attune NXT with a laser configuration of Blue (3)/Red (3)/Violet (4)/Yellow (4)) equipped with a high-throughput autosampler (THERMOFISHER CytKick). Compensation is performed using UltraComp eBeads (THERMOFISHER 01-3333-41), ArC Amine Reactive Compensation Bead Kit (THERMOFISHER A10346), and Luciferase/tdTomato Dual-Reporter HEK293 cells (Alstem LRL01). Analysis is performed using FLOWJO software (BD V10.8.1). Cells are identified with markers in Table 7A and tdTomato gates placed so that the negative control would be ≤0.5%⁺.

To determine the cell chimerism and frequency of tdTomato in the recipients, whole blood samples 1.5 μL are incubated in 96-well U-bottom plate which contains 50 μL/well Fc block for 5 mins at RT and then 50 μL/well antibody mastermix/FMOs on top of Fc block are added. Samples are incubated for 30 mins at RT in the dark prior to washing, then centrifuged at 1000×g for 5 mins with 100 μl of staining buffer. Samples are washed again with wash buffer (200 μl) and the centrifugation step is repeated before acquiring on flow cytometer.

TABLE 7A
Definition of Cell Subsets in Mouse BM by Flow Cytometry

| Cell Type: | Surface Marker Phenotype: |
|---|---|
| CLP | L/DAqua−Lineage−Sca1+cKit^{lo}CD135+CD127+ |
| CMP | L/DAqua−Lineage−Sca1−cKit+CD16/32^{mid}CD34+ |
| GMP | L/DAqua−Lineage−Sca1−cKit+CD16/32^{hi}CD34+ |
| MEP | L/DAqua−Lineage−Sca1−cKit+CD16/32−CD34− |
| LSK | L/DAqua−Lineage−Sca1+cKit+ |
| LT-HSC | L/DAqua−Lineage−Sca1+cKit+CD34−CD135− |
| ST-HSC | L/DAqua−Lineage−Sca1+cKit+CD34+CD135− |
| MPP | L/DAqua−Lineage−Sca1+cKit+CD34+CD135+ |
| LT-HSC | L/DAqua−Lineage−Sca1+cKit+CD150+CD48− |
| (alternative)‡ | |
| ST-HSC | L/DAqua−Lineage−Sca1+cKit+CD150−CD48− |
| (alternative) | |
| MPP | L/DAqua−Lineage−Sca1+cKit+CD150+CD48+ |
| (alternative) | |

*Lineage is defined as including CD4, CD8a, CD11b, B220, GR1, Ter119.
‡HSCs gated two different ways, alternative denotes second strategy for gating specific HSC population.

TABLE 7B
Leukocyte surface antibody stains used for flow cytometry

| | | | | | | Usage |
|---|---|---|---|---|---|---|
| | | | | | | per test |
| Staining | | | | | | (dilution; |
| Step | Target | Fluor | Clone | Source | Cat No. | 1:X) |
| Live/Dead | Viability | Aqua | N/A | INVITROGEN | L34966 | 1000 |
| Surface | Super Bright Staining Buffer | | | eBioscience | SB-4401-75 | 20 |
| | Fc Block | N/A | S17011E | BIOLEGEND | 156604 | 200 |
| | CD49b | AF488 | DX5 | BIOLEGEND | 108913 | 200 |
| | CD11b | PerCP/Cy5.5 | M1/70 | BIOLEGEND | 101228 | 200 |
| | CD45.2 | APC | 104 | BIOLEGEND | 109814 | 100 |
| | Ly6G | AF700 | 1A8 | BIOLEGEND | 127622 | 200 |
| | CD11c | APC/Fire750 | N418 | BIOLEGEND | 117352 | 200 |
| | CD19 | BV421 | 6D5 | BIOLEGEND | 115538 | 200 |
| | TCRb | PE/Cy7 | H57-597 | BIOLEGEND | 109222 | 200 |
| | IA/IE | BV605 | M5/114.15.2 | BIOLEGEND | 107639 | 500 |
| | CD45.1 | BV711 | A20 | BIOLEGEND | 110739 | 200 |
| Reporter | tdTomato | tdTomato | N/A | N/A | N/A | N/A |
| (not stained) | | | | | | |

TABLE 7C
Whole blood surface antibody stains used for flow cytometry

| | | | | | | Usage |
|---|---|---|---|---|---|---|
| | | | | | | per test |
| Staining | | | | | | (dilution; |
| Step | Target | Fluor | Clone | Source | Cat No. | 1:X) |
| Live/Dead | Viability | Aqua | N/A | INVITROGEN | L34966 | 1000 |
| Surface | Super Bright Staining Buffer | | | eBioscience | SB-4401-75 | 20 |
| | Fc Block | N/A | S17011E | BIOLEGEND | 156604 | 200 |
| | CD45.2 | APC | 104 | BIOLEGEND | 109814 | 100 |
| | CD71 | APC/Fire750 | RI7217 | BIOLEGEND | 113828 | 100 |
| | CD45.1 | BV421 | A20 | BIOLEGEND | 110732 | 100 |
| | CD41 | BV605 | MWReg30 | BIOLEGEND | 133921 | 100 |
| | CD44 | BV711 | IM7 | BIOLEGEND | 103057 | 100 |
| | Ter119 | PE/Cy7 | TER119 | BIOLEGEND | 116222 | 100 |
| Reporter | tdTomato | tdTomato | N/A | N/A | N/A | N/A |
| (not stained) | | | | | | |

### Example 7B: Adoptive Transfer of Bone Marrow after In Vivo LNP Delivery of Cre mRNA

### Primary Bone Marrow Transplantation

Female Ai14 mice (B6.Cg-Gt (ROSA) 26Sortm14 (CAG-tdTomato) Hze/J (Jackson Laboratory 007914) CD45.2 background) were dosed with LNP formulations (PBS control, F-70 and F-71) prepared as described in Example 2A at a dosage of 0.5 mg/kg i.v., serving as primary donors. At 2 weeks post dose, Ai14 donors were euthanized, and the femurs and tibiae were harvested, flushed out with RPMI (Gibco 72400-047) containing 0.5% BSA (MILTENYI 130-091-376), filtered through a 70 μm cell strainer (MILTENYI 130-098-462) and then centrifuged (500 g, 3 min). The resulting cell pellet was subjected to RBC lysis using ACK buffer (Thermo Fisher A1049201) for 5 min, diluted with RPMI containing 0.5% BSA (CRPMI), filtered, centrifuged, and resuspended in cRPMI and counted. A fraction of BM cells were stained for flow cytometry (see Flow Cytometry section in Example 7 for appropriate procedure) to determine the frequency of tdTomato in donor cell fractions. The remaining bone marrow cells were washed with PBS, re-counted, and resuspended in sterile PBS (for example, at 5×10⁶/200 μl PBS, 5×10⁶ total BM cells) and were adoptively transferred into immunodeficient NBSGW primary recipient mice (NOD.Cg-Kit^{W-41J}Tyr⁺Prkdc^{scid}[II2rgtm] Wj1/ThomJ, CD45.1 background). Primary recipient mice were survival bled bi-weekly/monthly for up to 12 weeks and analyzed for myeloid and lymphoid cell chimerism and frequency of tdTomato by flow cytometry. Leukocytes were also collected for analysis of tdTomato frequency in immune cells (B cells, T cells, NK cells, neutrophils, etc.)

### Secondary Bone Marrow Transplantation

The above procedure was repeated after 16 weeks, utilizing the primary bone marrow transplant recipients as donors. Lineage negative cells from the bone marrow of the primary recipient mice were magnetically isolated and then transferred to a second set of recipient mice (immunodeficient NBSGW mice). Secondary recipient mice were survival bled bi-weekly/monthly for up to 16 weeks and analyzed for myeloid and lymphoid cell chimerism and frequency of tdTomato by flow cytometry. At the end of 16 weeks, the secondary recipient mice were terminated and bone marrow cells were collected. Very low tdTomato expression was measured in the secondary recipient mice after 16 weeks. Without intending to be limited to any particular theory, it is believed that the low amount of observed expression in the bone marrow of the secondary recipients was likely due to the low yield of lineage negative cells from the primary recipient mice, and engraftment in the secondary transplantation was not optimal. Repeated experiments with a large pool of isolated lineage negative cells for secondary transplantation is expected to demonstrate greater engraftment and expression.

### Flow Cytometry Staining

To determine the frequency of tdTomato in the donor bone marrow cells, ~10 million cells per well are plated in a 96-well bottom plate, stained in 1×PBS with Live/Dead Fixable Aqua (INVITROGEN L34966) at 1:1000 for 20 min at room temperature. Cells are then washed twice with Cell Staining Buffer (BIOLEGEND 420201) and incubated with labeled CD16/32 antibody (bone marrow) for 5 min at 4° C. and surface antibody stains is added on top of the Fc Block for an additional 30 min at 4° C. Cells are then washed three times with Cell Staining Buffer and fixed with CYTOFIX (BD 554655) at 4° C. for 30 min. Cells are washed twice with 1×PBS and filtered through a 30-40 μm filter (Pall 8027) and acquired on a cytometer (THERMOFISHER Attune NXT with a laser configuration of Blue (3)/Red (3)/Violet (4)/Yellow (4)) equipped with a high-throughput autosampler (THERMOFISHER CytKick). Compensation is performed using UltraComp eBeads (THERMOFISHER 01-3333-41), ArC Amine Reactive Compensation Bead Kit (THERMOFISHER A10346), and Luciferase/tdTomato Dual-Reporter HEK293 cells (Alstem LRL01). Analysis is performed using FLOWJO software (BD V10.8.1). Cells are identified with markers in Table 7A and tdTomato gates placed so that the negative control would be ≤0.5%⁺.

To determine the cell chimerism and frequency of tdTomato in the recipients, whole blood samples (1.5 μL) are incubated in 96-well U-bottom plate containing 50 μL/well Fc block for 5 mins at RT and then 50 μL/well antibody mastermix/FMOs on top of Fc block were added. Samples were incubated for 30 mins at RT in the dark prior to washing, then centrifuged at 1000×g for 5 mins with 100 μl of staining buffer. Samples were washed again with wash buffer (200 μl) and the centrifugation step was repeated before acquiring on flow cytometer.

Results: Increased tdTomato Frequency Over Time in Blood Lineage Cells

As described above, primary recipient mice were survival bled bi-weekly/monthly for up to 12 weeks and analyzed for myeloid and lymphoid cell chimerism and frequency of tdTomato by flow cytometry. A small portion of whole blood samples was used for evaluating tdTomato signals in red blood cell (RBC)/platelets compartments as described above. Cells were identified with markers in Tables 7D. The rest of the blood samples were lysed with RBC lysis buffer at RT for 10 mins and then washed with buffer prior to Live/Dead staining, Fc blocking and surface antibody staining. Lymphocytes (B and T cells) and neutrophils were identified with markers in Tables 7D. Different lineage cell types including RBC, platelets, B, T cells and neutrophils showed a significant increase in tdTomato positive cell percentage over time (Table 7E), suggesting that the engrafted donor hematopoietic stem cells maintained progenitor cell potential and were able to differentiate to downstream lineage cells after transplantation.

TABLE 7D
Definition of Cell Subsets in Mouse whole blood
and spleen by Flow Cytometry used in Example 7B

| Cell type | Surface marker phenotype: |
|---|---|
| Erythrocyte | L/D Aqua− CD45− FSC^{lo} CD41− |
| progenitors/ | Ter119+ CD71+ Ter119− |
| Immature red | |
| blood cells | |
| Red blood cells | L/D Aqua− CD45− FSC^{hi} CD41− |
| | Ter119+ CD71− Ter119+ |
| Platelets | L/D Aqua− CD45− FSC^{hi} CD41+ Ter119− |
| Engraftment (CD45.2) | L/D Aqua− Ter119− CD45.2+ |
| T cell | L/D Aqua− Ter119− CD45.2+ CD19− TCRb+ |
| B cell | L/D Aqua− Ter119− CD45.2+ CD19+ TCRb− |
| Neutrophils | L/D Aqua− TCRb− CD19− Ly6g+ CD11b+ |
| NK cells | L/D Aqua− TCRb− CD19− Ly6G− CD49b+ |
| Monocytes | L/D Aqua− TCRb− CD19− Ly6G− CD11b+ |

TABLE 7E
tdTomato frequency (%) in different cell types over time
post primary transplantation (Average from 10 animals)

| | | | |
|---|---|---|---|
| Ctrl C57/Bl6 | Platelets | Immature RBCs | RBCs |

Whole blood

| | | | | |
|---|---|---|---|---|
| Week 2 | 0 | 1.8 | 12.3 | 2.4 |
| Week 4 | 0 | 5.7 | 18.2 | 10.6 |
| Week 8 | 0 | 9.7 | 15.6 | 14.8 |
| Weck 12 | 0 | 14.2 | 24.9 | 18.14 |

Leukocytes

| | | | | |
|---|---|---|---|---|
| Week 2 | 0 | 7.5 | 7.7 | 17.8 |
| Week 4 | 0 | 15 | 12 | 44.2 |
| Week 8 | 0 | 17.9 | 16.2 | 38.8 |
| Week 12 | 0 | 22.7 | 19.7 | 58.6 |

### Full Hematopoietic Reconstitution Upon Primary BM Transplantation

Multiple tissues from primary recipients were harvested 12-week post transplantation for evaluating hematopoietic reconstitution and donor cell engraftment. Harvested spleens were dissociated into single cell suspension of splenocytes using the gentleMACS Octo Dissociator with Heaters with a Mouse Spleen Dissociation Kit per manufacturer's instructions. Dissociated splenocytes were then passed through a 70 μm filter and washed with 1×PBS containing 2 mM EDTA and 0.5% BSA. Red blood cells were lysed using ACK Lysing Buffer and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA and counted. Cells were diluted, plated, and stained for flow cytometry. Bone marrow (BM) cells were harvested, passed through a 70 μm filter, and washed with 1×PBS+2 mM EDTA+0.5% BSA. Red blood cells were lysed using ACK Lysing Buffer and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA and counted. Cells were diluted, plated, and stained for flow cytometry. Blood samples were harvested and RBC lysed using ACK lysing buffer and washed twice with 1×PBS+2 mM EDTA+0.5% BSA, passing the cell suspension through 70 μm filter prior to flow staining. Briefly, cells were stained with Live/Dead Fixable Aqua, incubated with Fc block for 5 min at 4° C. and surface antibody stains (panel shown below in Table 7C). Cells were then washed and filtered and acquired on cytometer (THERMOFISHER Attune NXT or Sony ID7000 Cytometer) equipped with a high-throughput autosampler. Analysis performed using FLOWJO software (BD V10.8.1). Cells were identified with markers in Tables 7D. Flow cytometry was used to define donor cells based on the congenic marker CD45.2 expression. In Spleen and Blood, very high engraftment was observed (Table 7F) indicating high efficiency of CD45.2 donor cell reconstitution to blood lineage cells. Some variabilities were observed in bone marrow samples with an average of 70-80% engraftment across all three test groups.

TABLE 7F
CD45.2 congenic marker frequency (%) within each organ

| Test Article | Bone marrow | Spleen | Blood |
|---|---|---|---|
| Control | 86.8 | 99.2 | 99.7 |
| F-71 | 83 | 99 | 98.4 |
| F-70 | 68.6 | 89.3 | 96.6 |

tdTomato Frequency in Spleen and Bone Marrow 12 Week Post Primary Transplantation

To evaluate the frequency of tdTomato positive cells in spleen and bone marrow of the primary recipients, tissues were harvested and processed as described above. 10 million bone marrow cells and 5 million splenocytes were subjected to flow cytometry running analysis. tdTomato positive cells in T, B, Monocyte, and neutrophils were analyzed. 10-20% of these populations showed tdTomato positive, indicating that these cells were differentiated from tdTomato positive progenitors. Similarly, 10-20% of different bone marrow subsets showed tdTomato signals for mice receiving donations from test subject administered with F-70 (Table 7G-H). No tdTomato positive cells were observed in the F-71 or control groups.

TABLE 7G
Frequency of tdTomato positive cells (%) in
spleen 12 week post primary transplantation

| Test Article | T cells | B cells | Monocytes | Neutrophils |
|---|---|---|---|---|
| F-70 | 12.9 | 14.13 | 21.5 | 21 |

TABLE 7H
Frequency of tdTomato positive cells (%) in bone
marrow 12 week post primary transplantation

| | Bulk LSK | LT-HSC | ST-HSC | MPP | CMP | GMP | MEP |
|---|---|---|---|---|---|---|---|
| F-70 | 11.7 | 7.9 | 2.9 | 28.8 | 9.2 | 14.8 | 16.4 |

### Example 8: In Vitro Editing of CD34+ Peripheral Blood Cells Using LNPs Carrying Cas9+gRNA

LNP formulations containing editing cargo (Cas9 mRNA+/−gRNA targeting BCL11a erythroid enhancer) were prepared as described in Example 2D. LNPs were pre-incubated with 20 μg/ml ApoE3 at 4° C. overnight. CD34+ mobilized peripheral blood cells were co-cultured with pre-treated LNPs in X-VIVO media containing 100 ng/ml TPO, 100 ng/ml FLT3L and 100 ng/ml SCF. Cells with LNP were incubated at 37° C. in 5% CO₂. For samples containing editing cargo, one day after co-culture, LNPs were washed out. Four days after co-culture, cell pellets were collected for genomic DNA isolation (Microgem PrepGem Universal kit following manufacturer's protocol) and downstream NGS analysis (performed as described in Example 5). The percentage of indels at the BCL11a locus for each test article are shown in Table 8A.

TABLE 8A
Percentage of Indels at the BCL11a erythroid enhancer location

| | | |
|---|---|---|
| Form. | LNP amount | Indel % |

| | | | |
|---|---|---|---|
| F-52* | 5 | μg | 0.01 |
| F-52 | 5 | μg | 64.99 |
| F-52 | 9.5 | μg | 72.07 |

Values shown are mean representing n=2 replicates.

### Example 9: In Vitro Editing of HSPC Cells Using LNPs Carrying Cas9+gRNA

LNP formulations were prepared as described in Example 2D and 2E. In order to optimize lipid nanoparticle (LNP) formulations for hematopoietic stem and progenitor cells (HSPCs), a reporter-based (eGFP) positive screen using an exemplary ionizable lipid, formulated with either PEG-DMG, PEG-DPPE, and PEG-DSPE at varying percent (%) molarity (Formulations F-58 through F-69 of Example 2E). Briefly, HSPCs from three donors (AllCells Inc., Donor A, Donor B, and Donor C) in HSPC media (X-VIVO 15 (Lonza Inc, Cat. No. 02-053Q), supplemented with 100 ng/ml Stem Cell Factor (SCF, Peprotech, Cat. No. 300-07), Thrombopoietin (TPO, Peprotech, Cat. No. 300-18), and Flt3-Ligan (Flt3, Peprotech, Cat. No. 300-19) were plated at a density of 1×10⁶ HSPCs/ml for two days in a T-75 cell culture flask. Subsequently, HSPC viability was assessed using the Vi-CELL BLU (Beckman Coulter Inc.) and normalized to 2×10⁶ HSPCs/ml in HSPC transfection media (HSPC media, supplemented with 1 μg/ml ApoE3 (Peprotech, Cat. No. 350-02). Next, 1×10⁵ HSPCs were transferred to a round bottom 96-well tissue culture plate containing 50, 100 or 200 ng of LNP and incubated overnight (16 hours). The following morning, HSPCs were assessed for eGFP positivity (% cGFP) and mean-fluorescence intensity (MFI), on an Attune NxT Flow Cytometer.

Based on reporter expression data from the assays reported above, five LNP formulations (Table 9A) were selected for in vitro BCL11A-targeting gene editing cargo formulation. Cell culture procedures of HSPC gene editing LNP transfections were analogous to the procedures described above for the reporter assay, with the modification of a 2-day incubation period after transfection, in order to account for the kinetics of in vitro InDel formation. Without intending to be limited to any particular theory, the data demonstrate that LNP formulations of the present disclosure are capable of delivering a payload for editing at the clinically relevant BCL11A locus. Additionally, and further without intending to be limited to any particular theory, LNP formulations demonstrating superior efficacy for delivery to cells in vitro may not be the optimal formulation for in vivo methods of treatment. In certain embodiments, LNP formulations with a higher mol % of PEG lipid (2-5 mol %) are better suited for in vivo delivery to HSPCs.

TABLE 9A
Percentage of Indels at the BCL11a erythroid enhancer location

| Formulation | Donor A | Donor B | Donor C |
|---|---|---|---|
| F-53 | 12.2 | 23.6 | 20.9 |
| F-54 | 28.8 | 46.9 | 37.3 |
| F-55 | 41.8 | 58.2 | 53.1 |
| F-56 | 55.0 | 67.9 | 67.9 |
| F-57 | 13.1 | 20.02 | 12.4 |

### Example 10: LNP Delivery of VHH mRNA to HSCs-BALB/c Mice

LNP formulations were prepared as described in Example 2F. Each formulation was administered to BALB/c mice on Day 1, injected via tail vein. 16-24 h post injection, animals were euthanized by CO₂ inhalation, and spleens, femurs, and tibiae/fibulae were harvested. Harvested spleens were dissociated into single cell suspension of splenocytes using the gentleMACS Octo Dissociator with Heaters with the Mouse Spleen Dissociation Kit per manufacturer's instructions. Dissociated splenocytes were then passed through a 70 μm filter and washed with 1×PBS containing 2 mM EDTA and 0.5% BSA. Red blood cells were lysed using ACK Lysing Buffer and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA and counted. Cells were diluted, plated, and stained for flow cytometry. Bone marrow (BM) cells were harvested, passed through a 70 μm filter, and washed with 1×PBS+2 mM EDTA+0.5% BSA. Red blood cells were lysed using ACK Lysing Buffer and washed twice with 1×PBS+2 mM EDTA+0.5% BSA, passing the cell suspension through an additional 70 μm filter prior to the last wash. Following final wash, cells were resuspended in 1×PBS+2 mM EDTA+0.5% BSA and counted. Cells were diluted, plated, and stained for flow cytometry. Briefly, cells were stained with Live/Dead Fixable Aqua, incubated with Fc block (splenocytes) or labeled CD16/32 antibody (bone marrow) for 5 min at 4° C. and surface antibody stains (panel shown below in Table 10B). Cells were then washed and filtered and acquired on cytometer (THERMOFISHER Attune NXT or Sony ID7000 Cytometer) equipped with a high-throughput autosampler. Analysis performed using FLOWJO software (BD V10.8.1). Cells were identified with markers in Tables 8A. Flow cytometry was used to define long-term hematopoietic stem cells (LT-HSC) based on the following: Viable, lineage negative, Sca-1+, c-Kit+, CD150+, CD48−. The percentage of LT-HSCs and LSK cells expressing VHH is reported below in Table 10C. Several LNP formulations demonstrated high delivery (>40%) to HSPCs. Specifically, high delivery by many of the LNP formulations to LT-HSCs strongly suggests that delivery of gene editing systems in vivo can be used to treat diseases related to hematopoietic stem cells, such as hemoglobinopathies, in a subject in need thereof.

TABLE 10A
Definition of Cell Subsets in Mouse BM
by Flow Cytometry used in Example 10

| Cell Type: | Surface Marker Phenotype: |
|---|---|
| LSK | L/DAqua−Lineage−Sca1+cKit+ |
| LT-HSC | L/DAqua−Lineage−Sca1+cKit+CD150+CD48− |

*Lineage is defined as including CD4, CD8a, CD11b, B220, GR1, Ter119.

TABLE 10B
Bone marrow surface antibody stains used in Example 10

| Staining Step | Target | Clone | Target | Clone |
|---|---|---|---|---|
| Live/Dead | Viability | N/A | | |
| Surface Marker | CD16/32 | S17011E | CD150 | TC15-12F12.2 |
| | cKit | 2B8 | CD4 | GK1.5 |
| | CD135 | A2F10 | CD8a | 53-6.7 |
| | CD34 | RAM34 | CD11b | M1/70 |
| | CD48 | HM48-1 | B220 | RA3-6B2 |
| | Sca1 | D7 | Gr1 | RB6-8C5 |
| | CD127 | A7R34 | Ter119 | TER119 |

TABLE 10C
Percentage of cells expressing VHH mRNA

| Form | LSK | LT-HSC |
|---|---|---|
| F-Z1 | ** | *** |
| F-Z5 | * | * |
| F-Z10 | ** | *** |
| F-Z11 | *** | *** |
| F-Z12 | ** | *** |
| F-Z14 | *** | **** |
| F-Z16 | ***** | **** |
| F-Z17 | *** | *** |
| F-Z20 | **** | ***** |
| F-Z22 | ***** | ***** |
| F-Z23 | ** | ** |

Key: * < 5%; 5% ≤ ** < 25%; 25% ≤ *** < 50%; 50% ≤ **** < 75%; 75% ≤ ***** < 100%
LSK = Lin−Sca1+c-Kit+ hematopoietic stem cells;
LT-HSC = Long Term hematopoietic stem cells

### Example 11: LNP Delivery of VHH mRNA to HSCs-Humanized Mice

LNP formulations were prepared as described in Example 2F. Each formulation was administered to NBSGW mice that had previously been humanized by injection of human CD34+ cells. Animals were 12-20 weeks post-engraftment. LNPs were administered to mice on Day 1 injected via tail vein. 16-24 h post injection, animals were euthanized by CO₂ inhalation, and spleens, femurs, and tibiae/fibulae were harvested and processed as described in Example 10. Cells were diluted, plated, and stained for flow cytometry using surface antibody stains (panel shown below in Table 11B). Cells were then washed and filtered and acquired on cytometer (THERMOFISHER Attune NXT or Sony ID7000 Cytometer) equipped with a high-throughput autosampler. Analysis performed using FLOWJO software (BD V10.8.1). Cells were identified with markers in Tables 9A. The percentage of LT-HISCs and HSPCs expressing VIIII is reported below in Table 11C.

TABLE 11A
Definition of Cell Subsets in Mouse BM
by Flow Cytometry used in Example 11

| Cell Type: | Surface Marker Phenotype: |
|---|---|
| HSPC | L/DAqua−hCD45+Lineage−CD34+ |
| LT-HSC | L/DAqua−hCD45+Lineage−CD34+CD38−CD90+CD45RA− |

*Lineage is defined as including CD3, CD8, CD11b, CD11c, CD14, CD19, CD56.

TABLE 11B
Bone marrow surface antibody stains used in Example 11

| Staining Step | Target | Clone | Target | Clone |
|---|---|---|---|---|
| Live/Dead | Viability | N/A | | |
| Surface Marker | VIIII-GPI | 96A3F5 | CD235a | III264 |
| | Mouse CD45 | 30-F11 | CD3 | SP34-2 |
| | CD38 | OKT10 | CD8 | SK1 |
| | Human CD45 | HI30 | CD11b | ICRF44 |
| | CD34 | 561 | CD11c | 3.9 |
| | CD45RA | 5H9 | CD14 | M5E2 |
| | CD71 | M-A712 | CD19 | HIB19 |
| | CD90 | 5E10 | CD56 | 5.1H11 |

TABLE 11C
Percentage of cells of various types expressing VHH mRNA

| Form | HSPC | LT-HSC | Total CD34+ | HSC |
|---|---|---|---|---|
| F-Z1 | *** | *** | ** | ** |
| F-Z16 | *** | **** | *** | **** |
| F-Z20 | *** | ****** | ** | *** |
| F-Z22 | *** | **** | ** | **** |
| F-Z24 | | | *** | ***** |
| F-Z25 | | | **** | ***** |

Key: * < 5%; 5% ≤ ** < 25%; 25% ≤ *** < 50%; 50% ≤ **** < 75%; 75% ≤ ***** < 100%
HSPC = Hematopoietic stem and progenitor cells;
LT-HSC = Long Term hematopoietic stem cells

### Example 11B: LNP Delivery of VHH mRNA to Humanized CD34+ NSG Mice with Targeted LNPs

LNP formulations were prepared as described in Example 2G. CD34+ Humanized Mice (Hu-NSG, Jackson laboratory), were injected via tail vein with either 0.5 mg/kg or 1.0 mg/kg VHH mRNA formulated in LNP formulations F-76, F-77, F-78, F-79, and F-80 in a total volume of 5 mL/kg. Each formulation was dosed in 3 mice and an additional 1 mouse was dosed with PBS and an additional 2 mice were dosed with an fLuc mRNA LNP to serve as a control. 16 h post injection animals were euthanized by CO₂ inhalation, and tissues were harvested. Bone marrow (BM) was harvested from the bones by snipping the ends and spinning to remove BM. Red blood cells were lysed using ACK Lysing Buffer and washed twice with 1×PBS+2 mM EDTA+0.5% BSA, passing the cell suspension through a 70 μm filter prior to the last wash. Following final wash, cells were resuspended in 1×PBS+2 mM EDTA+0.5% BSA and counted. Cells were diluted, plated (10 million per well) in a 96-well bottom plate, and stained for flow cytometry. Briefly, cells were stained in 1×PBS with Live/Dead Fixable Aqua (INVITROGEN L34966) at 1:1000 for 20 min at room temperature. Cells were then washed twice with Cell Staining Buffer (BIOLEGEND 420201) and incubated with Fc block for 5 min at 4° C. and surface antibody stains either in full or FMO master mixes (panel and dilutions shown below in Table 11D) added on top of the Fc Block for an additional 30 min at 4° C. To note, staining was performed with either antibody clone of PE CD117 (104D2 or A3C6E2) to avoid crossblocking of receptor by CD117 clone conjugated to LNP formulation. Cells were then washed three times with Cell Staining Buffer and fixed with CYTOFIX (BD 554655) at 4° C. for 30 min. Cells were washed twice with 1×PBS and filtered through a 30-40 μm filter (Pall 8027) and acquired on a cytometer (THERMOFISHER Attune NXT with a laser configuration of Blue (3)/Red (3)/Violet (4)/Yellow (4) equipped with a high-throughput autosampler (THERMOFISHER CytKick). Compensation was performed using UltraComp eBeads and ArC Amine Reactive Compensation Bead Kit (THERMOFISHER A10346). Analysis was performed using FLOWJO software (BD V10.8.1). Cells were identified with markers in Table 11E and VHH reporter+ gates placed so that the negative control would be ≤0.5%⁺.

The percentage of cells of the noted varieties expressing VHH mRNA is reported below in Table 11F (as the mean of 3 animals). High levels of delivery were observed in primitive HSPC target cell populations with targeted LNP formulations F-78 and F-79 at both 0.5 mg/kg or 1.0 mg/kg doses.

TABLE 11D
Bone Marrow surface antibody stains used in Example 11B

| | | | | | Usage per test |
|---|---|---|---|---|---|
| Target | Fluorophore | Clone | Manufacturer | Cat No. | (dilution) |
| Cell Staining Buffer | | | BIOLEGEND | 420201 | |
| Super Bright | | | eBioscience | SB-4401-75 | 20 |
| Staining Buffer | | | | | |
| VHH-GPI | iFluor488 | 96A3F5 | GenScript | A01862 | 100 |
| Mouse CD45 | PerCP-Cy5.5 | 30-F11 | BIOLEGEND | 103132 | 200 |
| CD38 | APC | OKT10 | Caprico | 100846 | 50 |
| Human CD45 | APC/Fire750 | HI30 | BIOLEGEND | 304062 | 25 |
| CD34 | BV421 | 561 | BIOLEGEND | 343610 | 20 |
| CD45RA | BV605 | 5H9 | BD | 740424 | 50 |
| CD117 | PE | 104D2 | BIOLEGEND | 313204 | 20 |
| CD117 | PE | A3C6E2 | BIOLEGEND | 323408 | |
| CD90 | PE-Cy7 | 5.00E+10 | BIOLEGEND | 328124 | 20 |
| CD3 | AF700 | SP34-2 | BD | 557917 | 20 |
| CD8 | AF700 | SK1 | BIOLEGEND | 344724 | 20 |
| CD11b | AF700 | ICRF44 | BIOLEGEND | 301356 | 20 |
| CD11c | AF700 | 3.9 | BIOLEGEND | 301648 | 20 |
| CD14 | AF700 | M5E2 | BIOLEGEND | 301822 | 20 |
| CD19 | AF700 | HIB19 | BIOLEGEND | 302226 | 20 |
| CD56 | AF700 | 5.1H11 | BIOLEGEND | 362522 | 20 |
| CD235a | AF700 | HI264 | BIOLEGEND | 349122 | 20 |

TABLE 11E
Definition of Cell Subsets in humanized CD34+ NSG
mice Bone Marrow by Flow Cytometry used in Example 11B

| Cell Type: | Surface Marker Phenotype: |
|---|---|
| Lin−CD34+ | L/DAqua−huCD45+ Lineage−CD34+ |
| Lin−CD34+CD117+ | L/DAqua−huCD45+ Lineage−CD34+CD117+ |
| Lin−CD34+CD38− | L/DAqua−huCD45+ Lineage−CD34+CD38− |
| HSC | L/DAqua−huCD45+ Lineage−CD34+CD38−CD90+CD45RA− |
| MPP | L/DAqua−huCD45+ Lineage−CD34+CD38−CD90−CD45RA− |
| MLP | L/DAqua−huCD45+ Lineage−CD34+CD38−CD90−CD45RA+ |

*Lineage is defined as including CD3, CD8, CD14, CD19, CD56, CD11b, CD11c and CD235a

TABLE 11F
Percentage of cells of various types expressing VHH mRNA

| | Dose | Bone Marrow (@ 24 hr) | | | | | |
|---|---|---|---|---|---|---|---|
| Form | (mg/kg) | Lin-CD34+ | Lin-CD34+CD117+ | Lin-CD34+CD38− | HSC | MPP | MLP |
| F-80 | 1.0 | 0.23 | 0.38 | 0.23 | 0.91 | 0.17 | 0.19 |
| F-76 | 1.0 | 33.6 | 55.5 | 46.8 | 22.9 | 40.4 | 63.85 |
| F-77 | 1.0 | 34.9 | 60.4 | 51.3 | 26.5 | 44.4 | 62.1 |
| F-79 | 0.5 | 42.1 | 86.0 | 70.8 | 56.6 | 73.5 | 86.8 |
| F-79 | 1.0 | 57.7 | 97.1 | 81.6 | 87.8 | 78.3 | 92.4 |
| F-78 | 0.5 | 43.8 | 90.5 | 80.7 | 52.7 | 81.1 | 93.0 |
| F-78 | 1.0 | 64.0 | 98.6 | 95.5 | 86.3 | 96.2 | 97.9 |

### Example 12: LNP Editing in CD34+ Bone Marrow Progenitor Cells-Humanized Mice

LNP/mRNA formulations were prepared as described in Example 2D. Additional dosing formulations F-Q7 through F-Q10 containing Cas9 mRNA and gRNA, formulated as described in Example 2D were also tested with the following formulation parameters:

| | IL | PL | SL | PEG-L | Size (Z-avg | | EE | |
|---|---|---|---|---|---|---|---|---|
| Form | (mol %) | (mol %) | (mol %) | (mol %) | nm) | PDI | (%) | Buffer |
| F-Q7^{b} | AX-6 | DSPC + ESM | Chol | DSPE-PEG2k | 66.9 | 0.066 | 96.6 | Y |
| | (33%) | (20 + 20%) | (25%) | (2%) | | | | |
| F-Q8^{b} | AX-6 | DSPC + ESM | Chol | DPPE-PEG2k | 63.2 | 0.063 | 97.2 | Y |
| | (33%) | (20 + 20%) | (25%) | (2%) | | | | |
| F-Q9^{b} | AX-6 | DSPC + ESM | Chol | DMG-PEG2k | 82.2 | 0.04 | 94.5 | Y |
| | (33%) | (20 + 20%) | (25%) | (2%) | | | | |
| F- | AX-6 | ESM | Chol | DMG-PEG2k | 74.2 | 0.057 | 91.1 | Y |
| Q10^{b} | (33%) | (40%) | (25%) | (2%) | | | | |

Buffer Y: 50 mM Citrate, pH 4.0;
^{b}N:P = 8;
ESM = egg sphingomyelin; AX-6: an ionizable lipid comprising a protonatable tertiary amine head group and at least two biodegradable linkers.

Hu-CD34+ NSG (NOD.Cg-Prkdescid Il2rgtm1 Wj1, Jackson Laboratory 005557) were injected via tail vein with the indicated dosage of Cas9 mRNA plus BCL11A gRNA (Biospring) formulated in LNP formulations F-Q2 through F-Q10 in a total volume of 5 mL/kg. Control animals received LNP formulation F-Q1 that only contained Cas9 mRNA, lacking the gRNA. 5 days post injection, animals were euthanized by CO₂ inhalation, and spleens, femurs, and tibiae/fibulae were harvested. Some animals received a second dose at the same concentration 28 days after the first injection and then tissues were harvested on day 33; no additional editing was observed after the second dose in these animals. Bone marrow was removed from bones and red blood cells were lysed using ACK Lysing Buffer and then washed twice with 1×PBS+2 mM EDTA+0.5% BSA, passing the cell suspension through a 70 μm filter prior to the last wash. Following final wash, cells were resuspended in EasySep™ Buffer (StemCell 20144) and counted. Cells were diluted and plated (500 thousand per well) in a 96-well bottom plate for pre-enrichment purity check (Table 12C). Remaining cells had their concentration adjusted to 1×10⁸ cells/ml in EASYSEP™ Buffer (STEMCELL TECHNOLOGIES). Selection cocktail (StemCell 17856) was added to all samples at a concentration of 100 μl/ml and samples were incubated at room temperature for 10 minutes. RAPIDSPHERES™ was added to all samples at a concentration of 75 μl/ml and samples were incubated at room temperature for 5 minutes. Samples were transferred to Falcon Round-Bottom Polystyrene Tubes (CORNING 352052) during incubation and then placed in an EasySep™ magnet (STEMCELL 18000) for 3 minutes at room temperature. Supernatant was removed by inverting the tubes. Enriched cells were washed, counted, and plated in 96-well plate. Genomic DNA was isolated for downstream NGS analysis. Remaining enriched cells and flow through were purity checked by flow cytometry. Briefly, cells were stained in 1×PBS with Live/Dead Fixable Aqua (INVITROGEN L34966) at 1:1000 for 10 min at room temperature. Cells were then washed twice with Cell Staining Buffer (BIOLEGEND 420201) and incubated with Fc block for 5 min at 4° C. and surface antibody stains either in full or FMO (fluorescence minus one) master mixes (panels and dilutions shown below in Tables 12A) added on top of the Fc Block for an additional 30 min at 4° C. Cells were then washed and fixed. Cells were acquired on cytometer (SONY ID7000 Cell Analyzer: Cat #LE-ID7000C with UV/V/B/YG/R lasers). Single stain controls were made using UltraComp eBeads (THERMOFISHER 01-3333-41) and ArC Amine Reactive Compensation Bead Kit (THERMOFISHER A10346). Analysis was performed using FLOWJO software (BD V10.10.0). Cells were identified with the markers in Tables 12B.

CD34 purity was checked post enrichment, >95% purity was observed in all groups (Table 12C). NGS analysis of the enriched CD34+ HSPCs showed that Indels were detected in erythroid specific enhancer location with all LNP treatments that contained Cas9 mRNA plus BCL11A gRNA (Table 12D). Formulations utilizing the higher phospholipid content (40% cumulative phospholipid) demonstrated dose dependent editing response.

Spleens were harvested 5 days after dosing and flash frozen. Tissue was lysed with lysis master mix, containing TL Buffer, 1M DTT, and Proteinase K solution, shaking at 55° C. overnight. RNAse was added to samples and after centrifugation the supernatants were collected for genomic DNA isolation (MICROGEM PREPGEM UNIVERSAL™ kit following manufacturer's protocol) and downstream NGS analysis. Indel percentage was measured (Table 12E). Low (less than 1%) indel percentages were measured in splenocytes in all groups, suggesting low off-target editing.

TABLE 12A
Human Bone Marrow antibodies used in Example 12

| | | | | | | Usage per |
|---|---|---|---|---|---|---|
| Staining | | | | | | test |
| Step: | Target | Fluorophore | Clone | Manufacturer | Cat No. | (dilution) |
| Live/Dead | Viability | Aqua | N/A | INVITROGEN | L34966 | 1000 |
| Surface | Super Bright Staining Buffer | | | EBIOSCIENCE | SB-4401-75 | 20 |
| | HLA-A/B/C | AF488 | W6/32 | BIOLEGEND | 311413 | 20 |
| | Mouse CD45 | PerCP-Cy5.5 | 30-F11 | BIOLEGEND | 103132 | 200 |
| | CD38 | APC | OKT10 | CAPRICO | 100846 | 50 |
| | Human CD45 | APC/Fire750 | HI30 | BIOLEGEND | 304062 | 25 |
| | CD34 | BV421 | 561 | BIOLEGEND | 343610 | 20 |
| | CD45RA | BV605 | 5H9 | BD | 740424 | 50 |
| | B2M | PE | 2M2 | BIOLEGEND | 316306 | 20 |
| | CD90 | PE-Cy7 | 5E10 | BIOLEGEND | 328124 | 20 |
| | CD3 | AF700 | SP34-2 | BD | 557917 | 20 |
| | CD8 | AF700 | SK1 | BIOLEGEND | 344724 | 20 |
| | CD11b | AF700 | ICRF44 | BIOLEGEND | 301356 | 20 |
| | CD11c | AF700 | 3.9 | BIOLEGEND | 301648 | 20 |
| | CD14 | AF700 | M5E2 | BIOLEGEND | 301822 | 20 |
| | CD19 | AF700 | HIB19 | BIOLEGEND | 302226 | 20 |
| | CD56 | AF700 | 5.1H11 | BIOLEGEND | 362522 | 20 |
| | CD235a | AF700 | HI264 | BIOLEGEND | 349122 | 20 |

TABLE 12B
Definition of Cell Subsets in NHP BM aspirate
by Flow Cytometry used in Example 12

| Cell Type: | Surface Marker Phenotype: |
|---|---|
| Total CD34+ | L/DAqua−Mouse CD45−Human CD45+CD34+ |

TABLE 12C
Percentage of human CD34+ cells pre- and post-enrichment.

| | |
|---|---|
| Dose | Total CD34+ Cells |

| Form | (mg/kg) | Pre enrichment | Enriched CD34+ |
|---|---|---|---|
| PBS | — | 14.05 | 96.5 |
| F-Q1 | 1.5 | 8.14 | 95.63 |
| F-Q2 | 3.0 | 11.23 | 98.2 |
| F-Q4 | 3.0 | 13.1 | 97.6 |
| F-Q6 | 3.0 | 12.3 | 98.13 |
| F-Q7 | 2.0 | 10.65 | 92.57 |
| F-Q8 | 2.0 | 10.8 | 97.3 |
| F-Q9 | 1.0 | 12.9 | 96.9 |
| F-Q9 | 2.0 | 15.2 | 93.3 |
| F-Q9 | 3.0 | 11.4 | 92.3 |
| F-Q10 | 2.0 | 11.4 | 92.3 |

TABLE 12D
Percentage of Indels at the BCL11a erythroid enhancer location
in purified human CD34+ cells (Average of 3 animals)

| Form | Dose (mg/kg) | Indel % |
|---|---|---|
| PBS | — | 0.0 |
| F-Q1 | 1.5 | 0.02 |
| F-Q2 | 3.0 | 0.64 |
| F-Q4 | 3.0 | 1.25 |
| F-Q6 | 3.0 | 2.05 |
| F-Q7 | 2.0 | 9.16 |
| F-Q8 | 2.0 | 19.93 |
| F-Q9 | 1.0 | 4.6 |
| F-Q9 | 2.0 | 14.4 |
| F-Q9 | 3.0 | 18.7 |
| F-Q10 | 2.0 | 23.5 |

TABLE 12E
Percentage of Indels at the BCL11a erythroid specific
enhancer location in splenocytes (Average of 3 animals)

| Form | Dose (mg/kg) | Indel % |
|---|---|---|
| F-Q1 | 1.5 | 0.04 |
| F-Q2 | 3.0 | 0.15 |
| F-Q4 | 3.0 | 0.13 |
| F-Q6 | 3.0 | 0.1 |
| F-Q7 | 2.0 | 0.36 |
| F-Q8 | 2.0 | 0.68 |

### Example 13: Arginine Scanning Mutagenesis Results in Type V Editor Variants with Improved Function

Site-directed scanning mutagenesis is a useful tool applied in studying protein function and designing proteins with new properties, such as increased stability or enzymatic activity. In this experiment, a computational approach (“computational arginine scanning”) was used to develop a prioritized list of amino acid residues in the type V ortholog referred to herein as ID405 (SEQ ID NO: 52) which are predicted to have a meaningful interaction with a type V guide RNA. The identified positions were then engineered to be replaced with an arginine residue to create a series of arginine-containing variants. Without being bound by theory, the arginine residues (which carry a positive charge) are thought to provide a stronger interaction with the guide RNA at the substituted positions. The arginine-containing variants were then tested in an editing assay to determine whether editing efficiency was improved.

ID405 has the following amino acid sequence: (SEQ ID NO: 52).

### Step 1. Computational Arginine Scanning

Alpha Fold (open access AI based protein fold prediction software) was used to generate a three dimensional model of ID405. A tertiary complex of ID405, including both RNA and DNA was then created by superimposing the protein atoms of a homologous Cas12a (example 5b43.pdb) with AlphaFold-ID405 model and directly taking the nucleotide (eg “DNA” and “RNA”) atomic coordinates from the homolog.

The entire ID405 ribonucleic complex was minimized with standard modeling software (eg MOE, PyMoL, Rosetta) using the default energy function and varying dielectrics (examples D=2,3,4). A set of atoms (“DNA_or_RNA”) was created from the two bound nucleotide chains and a list of all contacts between the protein atoms and bound nucleotide atoms was created for a given distance threshold (examples distances could include 3 Å, 3.5 Å, 4 Å 4.5 Å). Individual atom contact energies for each amino acid within the distance threshold were summed and saved as the native nucleotide (NT) binding affinity score. For each residue within the threshold, standard modeling software (such as, but not limited to Rosetta, Schordinger, and Molecular Operating Environment (MOE)) was used to mutate each individual selected ID405 site in-silico to Arginine. Surrounding residues to the mutated site were repacked and additional minimization steps were performed if a large clash resulted. Resulting protein contacts and energetic scores between atoms of the site mutated to Arginine and any nucleotide were summed and recorded as the arginine nucleotide (NT) binding affinity score. The change in score between the starting wild type amino acid contacts and the mutated arginine amino acid were calculated and recorded as a “interchain ddG” or nucleotide binding affinity.

Independently, the delta in energetic contribution of each native amino acid within the distance threshold to the protein fold was also calculated before and after in silico mutation to arginine using standard modeling software and repacking neighboring residues (examples of standard software is as above). This score was recorded as the “intrachain ddG”.

Sites were sorted by magnitude of predicted interchain ddG (large negative values were favorable) and filtered such that the intra-chain ddG was less than a suitable threshold (values considered included <0, <1, <2, <3, <4 and <5). Residues that were native Arginine were ignored. Based on this criteria, 24 sites were prioritized for experimental validation, one of which (D169R) was already incorporated into ID405 as ID405-1 (SEQ ID NO: 53).

Top mutations selected computationally for experimental validation.
Arginine mutation is relative to the amino acid sequence of ID405-1.

| Native NT | Arginine | Arginine NT | Interchain | Intrachain | Average | |
|---|---|---|---|---|---|---|
| Affinity | Mutation | Affinity | ddG | ddG | Edits | # |
| −0.1 | Q492R | −30.0 | −29.9 | 2.2 | 76.7 | 1 |
| 9.0 | T306R | −17.5 | −26.5 | 1.7 | 69.7 | 2 |
| 0.0 | L748R | −23.6 | −23.6 | 1.7 | 4.6 | 3 |
| 0.0 | A739R | −21.9 | −21.9 | −1.2 | 62.7 | 4 |
| −1.3 | F297R | −20.0 | −18.7 | 4.0 | 67.9 | 5 |
| 0.7 | D169R | −16.5 | −17.2 | −5.1 | Inc. in 405-1 | 6 |
| 0.2 | N536R | −17.0 | −17.2 | 3.0 | 76.0 | 7 |
| −1.5 | D851R | −17.4 | −15.8 | 2.9 | 59.6 | 8 |
| −0.3 | N770R | −15.7 | −15.4 | 3.0 | 77.5 | 9 |
| 4.1 | D675R | −10.8 | −14.9 | 2.4 | 59.5 | 10 |
| −2.0 | K955R | −16.5 | −14.5 | −3.0 | 67.1 | 11 |
| 0.3 | S972R | −14.1 | −14.3 | 1.5 | 0.0 | 12 |
| 0.8 | N798R | −13.5 | −14.3 | −0.2 | 36.5 | 13 |
| −8.4 | K298R | −22.7 | −14.3 | 0.2 | 71.4 | 14 |
| −4.0 | H853R | −18.0 | −14.0 | 1.5 | 59.2 | 15 |
| −1.0 | E377R | −14.5 | −13.5 | 2.7 | 69.6 | 16 |
| 0.0 | V295R | −13.2 | −13.2 | 1.2 | 74.7 | 17 |
| −0.9 | K852R | −13.0 | −12.1 | 1.0 | 70.6 | 18 |
| −0.1 | F854R | −10.9 | −10.8 | −1.1 | 47.1 | 19 |
| −0.1 | N1015R | −10.4 | −10.3 | 4.3 | 80.0 | 20 |
| −3.3 | T845R | −13.2 | −9.8 | 1.3 | 73.8 | 21 |
| −1.5 | E1019R | −10.6 | −9.1 | −0.7 | 69.5 | 22 |
| −4.2 | K745R | −12.9 | −8.7 | 1.8 | 62.1 | 23 |
| −9.9 | K292R | −17.9 | −8.0 | 0.3 | 83.4 | 24 |

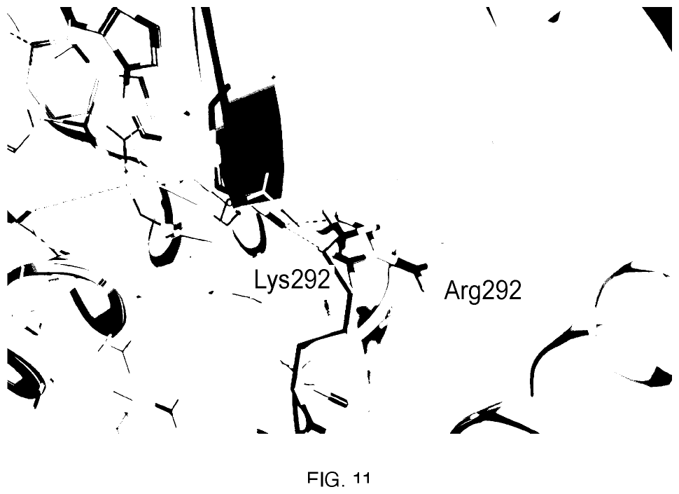

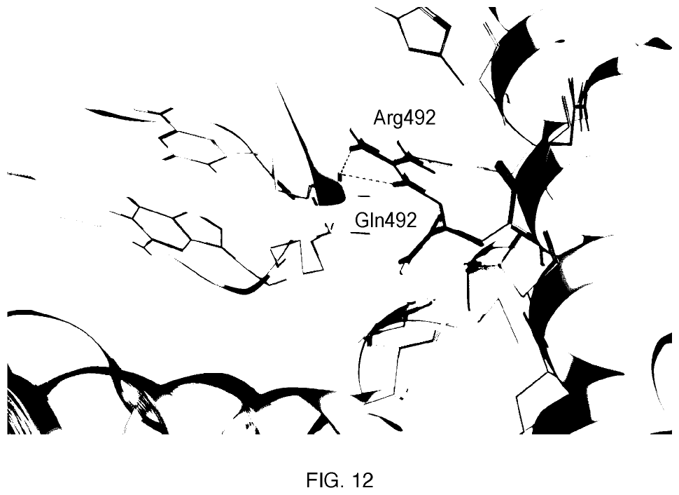

For illustrative purposes, FIG. 11 shows K292 amino acid native residue that is overlayed with the R292 substitution in the context of the three-dimensional structure of ID405-1 bound to a guide RNA. As can be inspected, the Arg292 residue provides for greater contacts (two dotted lines, versus a single dotted line) with the guide RNA.

In another example, FIG. 12 shows Q492 amino acid native residue that is overlayed with the R492 substitution in the context of the three-dimensional structure of ID405-1 bound to a guide RNA. As can be inspected, the Arg492 residue provides for greater contacts (two dotted lines, versus a no dotted line) with the guide RNA.

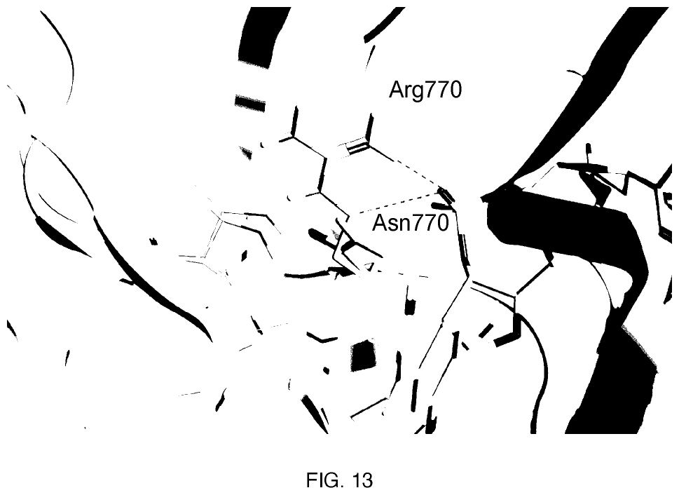

In yet another example, FIG. 13 shows N770 amino acid native residue that is overlayed with the R770 substitution in the context of the three-dimensional structure of ID405-1 bound to a guide RNA. As can be inspected, the Arg770 residue provides for greater contacts (two dotted lines, versus a no dotted line) with the guide RNA.

### Step 2. Experimental Testing of Select ID405-1 Arginine Substitution Mutants Leads to Improved Editing in Primary Cells

As described above in Step 1, a computational approach was undertaken to identify residues in ID405-1 that make contact sites with nucleic acid and which represent good candidates for site-specific substitution with arginine (positively charged) for enhancing said contact. It was reasoned that mutating these residues to arginine (R) could increase the affinity for guide RNAs and improve editing. The goal of this example is to design a variant ID405-1 variant with improved editing through systematic mutational analysis and to, in general, identify a strategy for improving the editing efficiency of any Cas nuclease, such as any type V Cas nuclease, including those disclosed herein.

### Results and Discussion

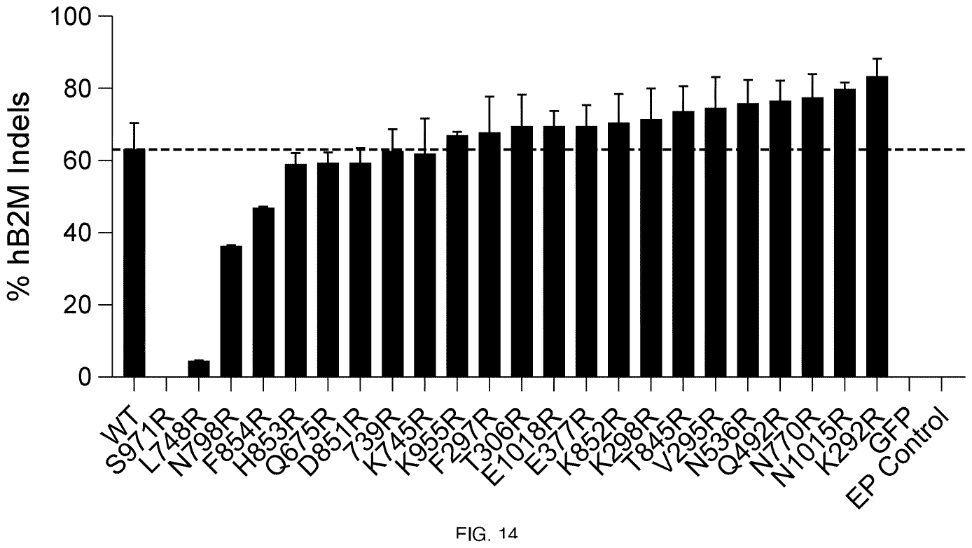

FIG. 14 shows the editing efficiency of the arginine variants constructed in Step 1 as measured in primary HSPCs. Cells were electroporated with RNA using the protocol detailed in the Materials and Methods section and 72 hours later samples were harvested for next generation sequencing (NGS) analysis. The benchmark control is termed WT and colored in yellow. This WT nuclease (ID405-1) edits the B2M locus at 63.4%. All mutants are in green/blue. Arginine substitution mutants identified in Step 1 that have editing efficiency below the yellow line are performing with lower editing efficiency than WT and those above are performing with higher editing efficiency than WT. For example, ID405-1 (K291R) performs with the highest editing efficiency at 83.4%.

This example demonstrates that arginine scanning—particularly where computation prediction is used first to predict the optimal residue for arginine replacement—can be used to improve the editing efficiency of a type V nuclease. This approach can also be used to improve the editing efficiency of other type V nucleases disclosed herein, as well as type II nucleases, or any nucleic acid programmable nuclease which forms a complex with a guide RNA.

Thus, in the context of type V ortholog ID405, this example demonstrates editing efficiency can be improved for nearly all of the arginine replacement variants tested, which are designated as follows: ID405 (Q492R); ID405 (T306R); ID405 (L748R); ID405 (A739R); ID405 (F297R); ID405 (D169R) [which is also designated herein as ID405-1]; ID405 (N536R); ID405 (D851R); ID405 (N770R); ID405 (D675R); ID405 (K955R); ID405 (S972R); ID405 (N798R); ID405 (K298R); ID405 (H853R); ID405 (E377R); ID405 (V295R); ID405 (K852R); ID405 (F854R); ID405 (N1015R); ID405 (T845R); ID405 (E1019R); ID405 (K745R); and ID405 (K292R).

In addition, this specification is not limited to arginine replacement variants of ID405, but contemplates arginine replacement variants of any type V nuclease ortholog disclosed herein wherein the replacements are at residue positions that correspond to the positions of Q492R; T306R; L748R; A739R; F297R; D169R; N536R; D851R; N770R; D675R; K955R; S972R; N798R; K298R; H853R; E377R; V295R; K852R; F854R; N1015R; T845R; E1019R; K745R; and K292R, as determined by a BLAST alignment between ID405 and the type V nuclease ortholog in which the substitutions are being made.

In addition, this specification contemplates variant type V orthologs that are engineered to contain two or more arginine replacements selected from the group consisting of: Q492R; T306R; L748R; A739R; F297R; D169R; N536R; D851R; N770R; D675R; K955R; S972R; N798R; K298R; H853R; E377R; V295R; K852R; F854R; N1015R; T845R; E1019R; K745R; and K292R, relative to the amino acid sequence of ID405, or at a corresponding residue positions in a type V ortholog of ID405 as determined by a BLAST alignment.

In certain embodiments, the specification contemplates a type V variant having a Q492R substitution (or equivalent substitution based on a BLAST alignment with ID405) could be pair up with one or more of the following substitutions: T306R; L748R; A739R; F297R; D169R; N536R; D851R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a T306R substitution (or equivalent substitution based on a BLAST alignment with ID405) could be pair up with one or more of the following substitutions: Q492R; L748R; A739R; F297R; D169R; N536R; D851R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a L748R substitution (or equivalent substitution based on a BLAST alignment with ID405) could be pair up with one or more of the following substitutions: Q492R; T306R; A739R; F297R; D169R; N536R; D851R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a A739R substitution (or equivalent substitution based on a BLAST alignment with ID405) could be pair up with one or more of the following substitutions: Q492R; T306R; L748R; F297R; D169R; N536R; D851R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a F297R substitution (or equivalent substitution based on a BLAST alignment with ID405) could be pair up with one or more of the following substitutions: Q492R; T306R; L748R; A739R; D169R; N536R; D851R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a D169R substitution (or equivalent substitution based on a BLAST alignment with ID405) could be pair up with one or more of the following substitutions: Q492R; T306R; L748R; A739R; F297R; N536R; D851R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a N536R substitution (or equivalent substitution based on a BLAST alignment with ID405) could be pair up with one or more of the following substitutions: Q492R; T306R; L748R; A739R; F297R; D169R; D851R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a D851R substitution (or equivalent substitution based on a BLAST alignment with ID405) could be pair up with one or more of the following substitutions: Q492R; T306R; L748R; A739R; F297R; D169R; N536R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a N770R substitution (or equivalent substitution based on a BLAST alignment with ID405) could be pair up with one or more of the following substitutions: Q492R; T306R; L748R; A739R; F297R; D169R; N536R; D851R; D675R; K955R; S972R; N798R; K298R; H853R; E377R; V295R; K852R; F854R; N1015R; T845R; E1019R; K745R; and K292R.

In certain embodiments, the specification contemplates a type V variant having a D675R substitution (or equivalent substitution based on a BLAST alignment with ID405) could be pair up with one or more of the following substitutions: Q492R; T306R; L748R; A739R; F297R; D169R; N536R; D851R; N770R; K955R; S972R; N798R; K298R; H853R; E377R; V295R; K852R; F854R; N1015R; T845R; E1019R; K745R; and K292R.

In certain embodiments, the specification contemplates a type V variant having a K955R substitution (or equivalent substitution based on a BLAST alignment with ID405) could be pair up with one or more of the following substitutions: Q492R; T306R; L748R; A739R; F297R; D169R; N536R; D851R; N770R; D675R; S972R; N798R; K298R; II853R; E377R; V295R; K852R; F854R; N1015R; T845R; E1019R; K745R; and K292R.

In certain embodiments, the specification contemplates a type V variant having a S972R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; N798R; K298R; H853R; E377R; V295R; K852R; F854R; N1015R; T845R; E1019R; K745R; and K292R.

In certain embodiments, the specification contemplates a type V variant having a N798R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; K298R; H853R; E377R; V295R; K852R; F854R; N1015R; T845R; E1019R; K745R; and K292R.

In certain embodiments, the specification contemplates a type V variant having a K298R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; H853R; E377R; V295R; K852R; F854R; N1015R; T845R; E1019R; K745R; and K292R.

In certain embodiments, the specification contemplates a type V variant having a H853R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; E377R; V295R; K852R; F854R; N1015R; T845R; E1019R; K745R; and K292R.

In certain embodiments, the specification contemplates a type V variant having a Q492R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; H853R; E377R; V295R; K852R; F854R; N1015R; T845R; E1019R; K745R; and K292R.

In certain embodiments, the specification contemplates a type V variant having a E377R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; H853R; V295R; K852R; F854R; N1015R; T845R; E1019R; K745R; and K292R.

In certain embodiments, the specification contemplates a type V variant having a V295R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; H853R; E377R; K852R; F854R; N1015R; T845R; E1019R; K745R; and K292R.

In certain embodiments, the specification contemplates a type V variant having a K852R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; H853R; E377R; V295R; F854R; N1015R; T845R; E1019R; K745R; and K292R.

In certain embodiments, the specification contemplates a type V variant having a F854R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; H853R; E377R; V295R; K852R; N1015R; T845R; E1019R; K745R; and K292R.

In certain embodiments, the specification contemplates a type V variant having a N1015R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; II853R; E377R; V295R; K852R; F854R; T845R; E1019R; K745R; and K292R.

In certain embodiments, the specification contemplates a type V variant having a K745R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; H853R; E377R; V295R; K852R; F854R; T845R; E1019R; and K292R.

In certain embodiments, the specification contemplates a type V variant having a K292R substitution (or equivalent substitution based on a BLAST alignment with ID405) could be pair up with one or more of the following substitutions: Q492R; T306R; L748R; A739R; F297R; D169R; N536R; D851R; N770R; D675R; K955R; S972R; N798R; K298R; H853R; E377R; V295R; K852R; F854R; T845R; E1019R; and K745R.

As to any of the type V variants contemplated by the method of this Example, this specification also contemplates type V proteins having at least 80%, or at least 85%, or at least 90%, or at least 95%, or at least 99%, or up to 100% sequence identity with any of the type V variants contemplated in this Example.

### Materials and Methods

### Mammalian Cell Culture

Primary human CD34+ HSPCs were cultured in X-Vivo 15 medium (Lonza Catalog #: 02-053Q) supplemented with 100 ng/mL SCF (RnD Systems Inc. 11010-SC-100), 100 ng/uL FLT-3L (RnD Systems Inc. 308-FKHB-250), and 100 ng/uL recombinant human thrombopoetin (RnD Systems Inc. 288-TPN-100/CF). HSPCs were thawed and cultured for three days before electroporation.

### Electroporation

HSPCs were electroporated using a Lonza 4D 96-well electroporator and a Lonza P3 Primary cell kit (Catalog #: V4SP-3096). Briefly, 100,000 HSPCs were resuspended in Lonza P3 buffer and combined with 6 ug RNA to a total volume of 20 μL unless otherwise noted. The samples were electroporated using the DS-130 program, 80 μL of pre-warmed cell culture medium was added to the cells immediately after electroporation, and the entire mixture transferred to a pre-warmed 96-well plate containing 100 μL of cell culture medium. Samples were allowed to grow at 37 C 5% CO₂ for three days before harvesting genomic DNA (gDNA) using prepGEM (MicroGem PUN1000) according to the manufacturer's instructions.

### Genomic DNA Extraction

After incubation, the media was removed from the cells and genomic DNA was extracted by the addition of prepGEM reagent (Thomas Scientific: PUN0050) directly into each well of the tissue culture plate. Lysed cells were transferred to a 96 well PCR plate and incubated at 75° C. for 10 minutes, followed by 95° C. enzyme inactivation step for 5 minutes.

### Next Generation Sequencing and Analysis

Using prepGEM lysates as template, the edited B2M site was amplified by PCR and prepared for Illumina sequencing. The resulting data was analyzed using CRISPREsso2. Editing outcome (InDels %) was calculated as the number of modified reads divided by total aligned reads.

### In Vitro Transcription (IVT) Reaction

Plasmids for IVT were made with gBlocks from IDT and NEB assembly. In vitro transcription performed using NEB HiScribe according to manufacturer's protocol using n1 methyl pseudouridine supplementation. NEB is New England Biolabs. IDT is Integrated DNA Technologies.

### Primers Used for NGS

Human B2M fwd:
(SEQ ID NO: 54)
CCTGAATTGCTATGTGTCTGGG
Human B2m rev:
(SEQ ID NO: 55)
CTCATACACAACTTTCAGCAGC

405-1 Targeting Spacer Used to Target Human B2M (gRNA from IDT)

(SEQ ID NO: 56)
AGTGGGGGTGAATTCAGTGT

Protein Sequences of Type V Arginine Substitution Variants Described in this Example.

ID405-1 (WT—arginine replacement locations in following sequences are relative to this sequence after the NLS/glycine linker shown in underlined text)) (SEQ ID NO: 57); ID405-1 (A738R) (SEQ ID NO: 58); ID405-1 (V294R) (SEQ ID NO: 59); ID405-1 (T844R) (SEQ ID NO: 60); ID405-1 (T305R) (SEQ ID NO: 61); ID405-1 (S971R) (SEQ ID NO: 62); ID405-1 (Q674R) (SEQ ID NO: 63); ID405-1 (Q491R) (SEQ ID NO: 64); ID405-1 (N1014R) (SEQ ID NO: 65); ID405-1 (N797R) (SEQ ID NO: 66); ID405-1 (N769R) (SEQ ID NO: 67); ID405-1 (N535R) (SEQ ID NO: 68); ID405-1 (L747R) (SEQ ID NO: 69); ID405-1 (K954R) (SEQ ID NO: 70); ID405-1 (K851R) (SEQ ID NO: 71); ID405-1 (K744R) (SEQ ID NO: 72); ID405-1 (K297R) (SEQ ID NO: 73); ID405-1 (K291R) (SEQ ID NO: 74); ID405-1 (H852R) (SEQ ID NO: 75); ID405-1 (F853R) (SEQ ID NO: 76); ID405-1 (F296R) (SEQ ID NO: 77); ID405-1 (E1018R) (SEQ ID NO: 78); ID405-1 (E376R) (SEQ ID NO: 79); ID405-1 (D850R) (SEQ ID NO: 80).

### Example 14: Modulation of NLS and Linker Configurations in a Type V Nuclease Improve Gene Editing in Primary Cells

### Background

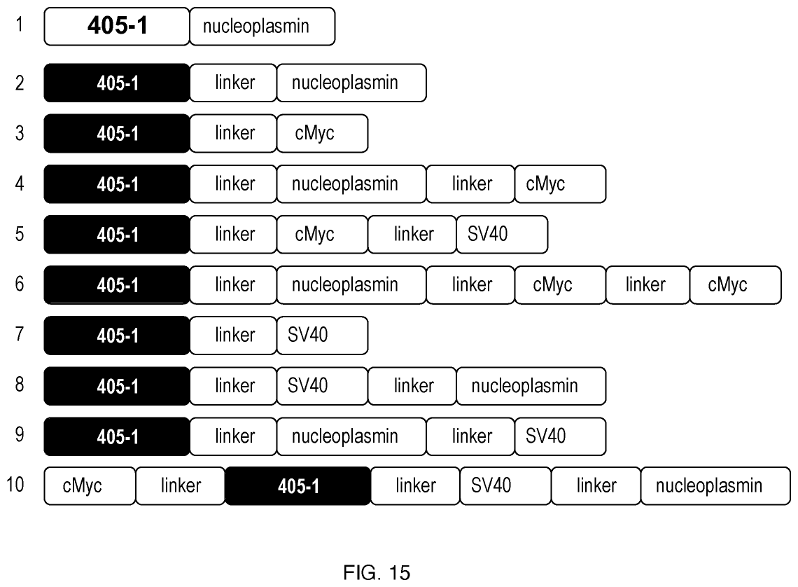

Recent advances in gene editing technologies have enabled the development of powerful tools for precise genome engineering. One such tool, Type V endonuclease, has garnered interest due to its smaller gRNA length and fewer off target indels. Editing using Type V endonuclease (and optimization thereof) has historically been done in the context of ribonucleoprotein (RNP). To the inventors' knowledge, Type V endonucleases have not previously been optimized in the context of an all-RNA system (mRNA nuclease and RNA guide), in particular, for in vivo delivery and editing. Protein activity, localization, and folding may be influenced by linkers, tags and NLS components attached the N- or C-terminus (Refs 1-3). In this example, 10 different construct configurations (see FIG. 15) of ID405-1 nuclease comprising various combinations of nuclear localization signals (NLSs) and linkers have been tested in primary cells. This example demonstrates that a type V nuclease, such as ID405-1, may be engineered with improved editing efficiency by introducing one or more NLS in various configurations.

### Results and Discussion

FIG. 15 illustrates the general structure of the ID405-1 variants constructed with one or more NLS and linkers. The figures shows the arrangement of the linkers and NLS used. The term “nucleoplasmin” refers to the nucleoplasmin NLS having the following sequence: KRPAATKKAGQAKKKK (SEQ ID NO: 81). The term “cMyc” refers to the cMyc NLS having the following sequence: PAAKRVKLD. The term “SV40” refers to the SV40 NLS having the following amino acid sequence: RSSDDEATADSQHAAPPKKKRKV (SEQ ID NO: 82). Construct 1 comprises a ID405-1 sequence fused directly to a nucleoplasmin NLS without a linker. The linker in all cases is 4 glycine (GGGG). Full sequences for all constructs are provided below. Plasmids used for IVT production of mRNA were synthesized by a DNA sequence vendor (e.g., Twist Biosciences) and IVT details are below.

Each of the constructs (mRNA encoding the recombinant ID405-1 variants and a guide RNA) were delivered by electroporation to primary HSPCs (AllCell). The nuclease mRNA is produced by IVT reaction and the guide RNA (spacer targeting hB2M) is synthetically manufactured by IDT. After transfection, cells were harvested 72 hours later in lysis buffer (as detailed in methods). Cells were analyzed by next-generation sequencing (NGS) and the editing efficiency was calculated.

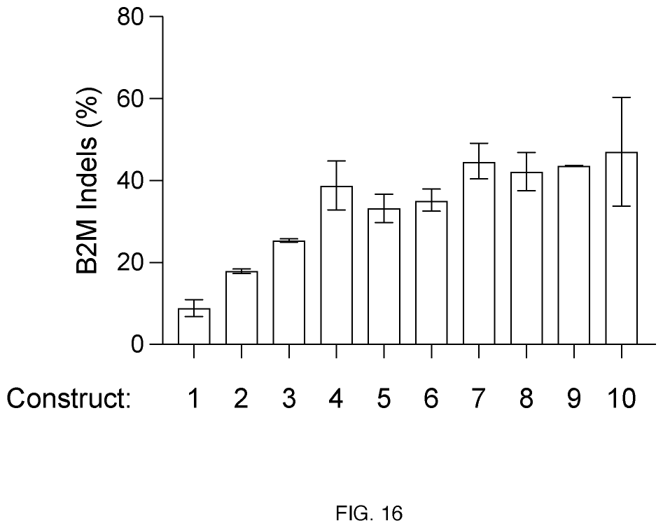

FIG. 16 shows the editing efficiency of the ID405-1 variants as % of sequencing reads showing an indel in the B2M locus. Construct 1 (405-1) edits the B2M locus at 8.9%. The data show that additional linkers and NLS significantly improve editing efficiency. Construct 10 performs best with 3 NLS which edits at 47%, a more than 5-fold increase.

The constructs engineered and tested in this Example are not intended to be limiting on this specification. The specification contemplates additional variant configurations and/or combinations of these modifications with other modifications, such as the arginine scan substitutions of Example 13.

### Materials and Methods

### Mammalian Cell Culture

Primary human CD34+ HSPCs were cultured in X-Vivo 15 medium (Lonza Catalog #: 02-053Q) supplemented with 100 ng/mL SCF (RnD Systems Inc. 11010-SC-100), 100 ng/uL FLT-3L (RnD Systems Inc. 308-FKHB-250), and 100 ng/uL recombinant human thrombopoetin (RnD Systems Inc. 288-TPN-100/CF). HSPCs were thawed and cultured for three days before electroporation.

### Electroporation

HSPCs were electroporated using a Lonza 4D 96-well electroporator and a Lonza P3 Primary cell kit (Catalog #: V4SP-3096). Briefly, 100,000 HSPCs were resuspended in Lonza P3 buffer and combined with 6 ug RNA to a total volume of 20 μL unless otherwise noted. The samples were electroporated using the DS-130 program, 80 μL of pre-warmed cell culture medium was added to the cells immediately after electroporation, and the entire mixture transferred to a pre-warmed 96-well plate containing 100 μL of cell culture medium. Samples were allowed to grow at 37 C 5% CO₂ for three days before harvesting genomic DNA (gDNA) using prepGEM (MicroGem PUN1000) according to the manufacturer's instructions.

Genomic DNA extraction After incubation, the media was removed from the cells and genomic DNA was extracted by the addition of prepGEM reagent (Thomas Scientific: PUN0050) directly into each well of the tissue culture plate. Lysed cells were transferred to a 96 well PCR plate and incubated at 75° C. for 10 minutes, followed by 95° C. enzyme inactivation step for 5 minutes.

### Next Generation Sequencing and Analysis

Using prepGEM lysates as template, the edited B2M site was amplified by PCR and prepared for Illumina sequencing. The resulting data was analyzed using CRISPREsso2. Editing outcome (InDels %) was calculated as the number of modified reads divided by total aligned reads.

IVT reaction

Plasmids for IVT were made with gBlocks from IDT and NEB assembly. In vitro transcription performed using NEB HiScribe according to manufacturer's protocol.

### Primers Used for NGS

Human B2M fwd:
(SEQ ID NO: 54)
CCTGAATTGCTATGTGTCTGGG
Human B2m rev:
(SEQ ID NO: 83)
CTCATACACAACTTTCAGCAGC

### 405-1 Targeting Spacer Used to Target Human B2M

(SEQ ID NO: 56)
AGTGGGGGTGAATTCAGTGT

### Protein Sequences of FIG. 15

Below are the amino acid sequences constructed in this example and as illustrated in FIG. 15. Each construct comprises (a) the ID405-1 amino acid sequence, (b) at least one NLS, and (c) optionally one or more linkers. For each construct below, NLS moieties are indicated with bold text and linkers, if present, are indicated in italicized text.

Construct 1 (RNA0465) (SEQ ID NO: 84); Construct 2 (RNA0821) (SEQ ID NO: 85); Construct 3 (RNA0824) (SEQ ID NO: 86); Construct 4 (RNA0823) (SEQ ID NO: 87); Construct 5 (RNA0825) (SEQ ID NO: 88); Construct 6 (RNA0827) (SEQ ID NO: 89); Construct 7 (RNA0819) (SEQ ID NO: 90); Construct 8 (RNA0820) (SEQ ID NO: 91); Construct 9 (RNA0822) (SEQ ID NO: 92); Construct 10 (RNA0826) (SEQ ID NO: 93).

### Example 15: Improved Type V Variants Obtained by Directed Evolution Approach

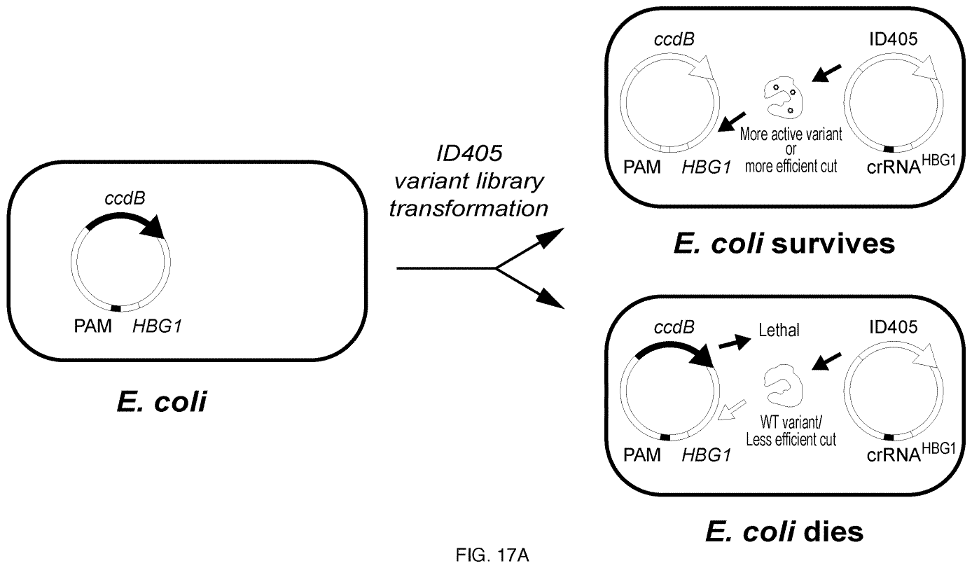

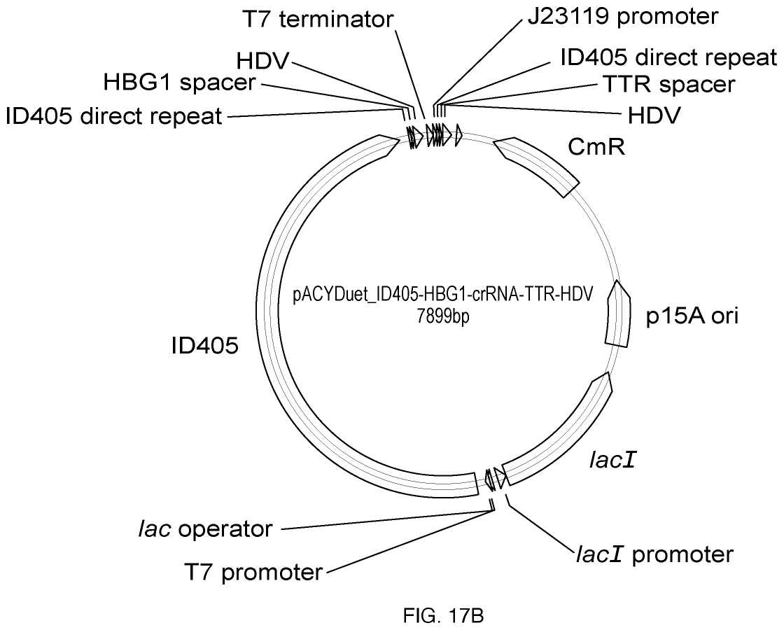

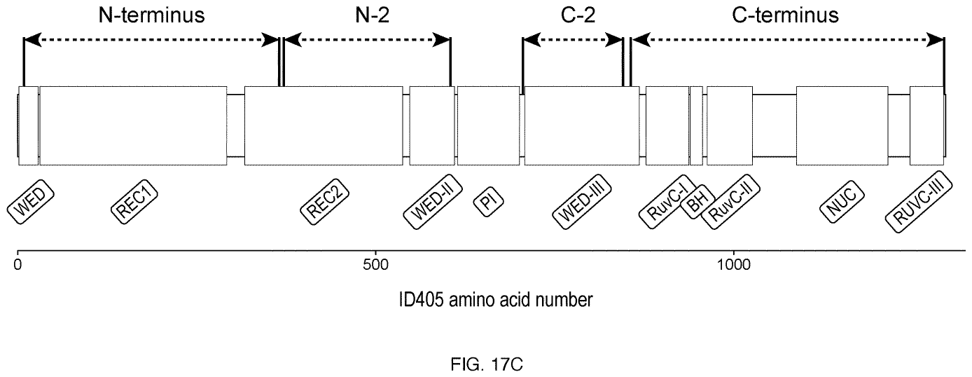

To increase the functional activity of ID405 on the HBG1 target in human cells, a strategy was contemplated whereby the activity of mutant Cas protein was linked to the survival of a E. coli host carrying plasmid with an L-arabinose inducible toxin gene as well as target sequence for Cas protein (similar to the model described in Zhang et al. 2021). The cleavage and disruption of ccdB plasmid allows cell survival after addition of L-arabinose. The selection strategy can be iterated many times by repeated extraction and transformation of mutant plasmids (selection rounds) thereby allowing the enrichment of the most efficient Cas variants (FIG. 17A).

Firstly, toxin ccdB gene was cloned with a modified ribosome binding sequence (RBS) under the L-arabinose inducible pBAD promoter (Chen and Zhao 2005) resulting in plasmid pBAD-*RBS_ccdB-lacY. Synthetic DNA fragments encoding ccdB and lacY genes were ordered from Twist Bioscience and cloned into a pBAD plasmid vector via Ncol and KpnI (both New England Biolabs) for ccdB, and Esp3I (New England Biolabs) for lacY. The ccdB gene encodes a protein that targets and inhibits DNA gyrase which is lethal for its bacterial host. The modified RBS allows for a more stringent control of ccdB gene expression (see Chen and Zhao 2005). Therefore, the default RBS sequence found in the pBAD vector was modified to GATTGA by Gibson assembly cloning using a NEBuilder Hi Fi DNA Assembly kit (New England Biolabs).

Downstream of the ccdB gene in separate plasmids, the ID405 target site HBG1 with two cloned with different PAM sequences: TCTT, TTGG denoting intermediate, and poor preference, respectively (Table 15A). Targets were cloned as annealed and phosphorylated oligo duplexes CZ-4734/CZ-4735, CZ-5082/CZ-5083 via KpnI/HindIII resulting in plasmids pBAD-*RBS-ccdB-TTGG-HBG1-lacY and pBAD-*RBS-ccdB-TCTT-HBG1-lac Y, respectively. Each plasmid was transformed into T7 Express Competent E. coli (High Efficiency) (New England Biolabs) according to manufacturer's protocol and plated onto LB media with 100 μg/mL carbenicillin. The transformants were grown in 300 mL of LB media supplemented with 100 μg/mL carbenicillin until mid-log, split into 6×50 mL falcons, washed three times with ice-cold 10% glycerol before concentrating 300-fold to prepare electrocompetent cells, and stored at −80° C.

TABLE 15A
ID405 target sites in pBAD plasmid
encoding ccdB toxin

| | | |
|---|---|---|
| Intermediate | TCTTCCTTGTCAAG | SEQ ID NO: 94 |
| PAM + target | GCTATTGGTCAAG | |
| Poor PAM + target | TTGGCCTTGTCAAG | SEQ ID NO: 95 |
| | GCTATTGGTCAAG | |

To generate ID405 containing plasmids, DNA fragments encoding the ID405 protein with the J23119 promoter sequence downstream were cloned into pACYC vectors via Ncol and PacI (both New England Biolabs) restriction enzymes. Both fragments were E. coli codon optimized and synthesized by Twist Bioscience. Subsequently, the HDV ribozyme sequence was added downstream of the crRNA cloning site by ligating via HindIII and PacI (both New England Biolabs) sites resulting in plasmid pACYCDuet_ID405-HDV. TTR fragment encoding crRNA was then cloned downstream J23119 promoter via BsaI using annealed and phosphorylated CZ-5151/CZ-5152 oligo duplex resulting in plasmid pACYCDuet_ID405-crRNA-TTR-HDV. The plasmid was amplified with primer pair CZ-5138/CZ-5086 and cloned via Gibson assembly using NEBuilder Hi Fi DNA Assembly kit (New England Biolabs) with HBG1 fragment amplified with CZ-4415/CZ-3621 resulting in plasmid pACYCDuet_ID405-HBG1-crRNA-TTR-HDV (FIG. 17B). As a control, a construct encoding LbaCas12a protein was constructed. Cloning scheme was the same except primer pair CZ-4402/CZ-4403 was used to generate pACYCDuet_Lba-crRNA-TTR-HDV, and primer pairs CZ-5085/CZ-5086, CZ-4417/CZ-3621 were used to generate plasmid pACYCDuet_Lba-HBG1-crRNA-TTR-HDV.

The TTR target, which is not present in bacteria, was chosen to titrate the activity of ID405 as in preliminary analysis the presence of unrelated crRNA reduces the activity against intended targets. The efficiency of ID405 to cut target sequences was tested by transforming this plasmid into T7 Express strains containing ccdB encoding plasmids with target sequences and plating onto LB medium containing 20 μg/mL chloramphenicol and 0.1% L-arabinose as described below.

To create an ID405 library with random mutations, domain structure of the protein (FIG. 17C) was inspected. A model was created using SWISS-MODEL workspace using default parameters (Guex, Peitsch, and Schwede 2009; Waterhouse et al. 2018). A PDB90 search was performed using DALI protein structure comparison server (Holm et al. 2023) and Acidaminococcus sp. BV3L6 Cpf1 (PDB accession numbers 5XH7 and 5XH6) was identified as the most similar characterized protein (Z-score=47) (Nishimasu et al. 2017). From structural alignment against the Cpf1 (PDB accession number 5XH6) the domain structure of ID405 (FIG. 17C) was inferred.

Having identified domains in ID405, the protein sequence was then split into 4 regions omitting PAM-interacting (PI) domains resulting the following fragments: N-terminus, C-terminus, N-2, C-2, each spanning 1072 base-pairs (bp), 1310 bp, 698 bp, 420 bp, respectively (FIG. 17C). Table 15B indicates the exact selected positions in the ID405 sequence that were mutagenized. The omittance of PI domain in the assay should allow the selection of more active protein variants against less preferred PAM sequences rather than versions with changed PAM recognition.

TABLE 15B
Selected ID405 regions for mutagenesis (in bp).
Note that mutation regions are calculated omitting
primer sequence from amplified region.

| | Start . . . End, |
|---|---|
| Region Name | nucleotide position |
| N-terminus | 23 . . . 1095 |
| N-2 | 1114 . . . 1812 |
| C-2 | 2118 . . . 2538 |
| C-terminus | 2572 . . . 3882 |

In the assay, the less preferred PAM sequences TCTT and TTGG were chosen, to create a stronger selection pressure for ID405 variants. Testing reactions were performed by electroporating E. coli cells containing pBAD-*RBS-ccdB-TCTT-HBG1-lac Y or pBAD-*RBS-ccdB-TTGG-HIBG1-lacY with 100 ng ID405 plasmid with or without IIBG1 spacer. Transformed bacteria were recovered in SOC medium for 1 h and 30 min with vigorous shaking at 37° C. Then, bacteria were plated on 3 separated LB medium plates containing 20 μg/mL chloramphenicol; 20 μg/mL chloramphenicol, 0.1% L-arabinose; and 20 μg/mL chloramphenicol, 0.1% L-arabinose, 0.1 mM IPTG. As a control, LbaCas 12a protein was used, cloned in the same manner as ID405.

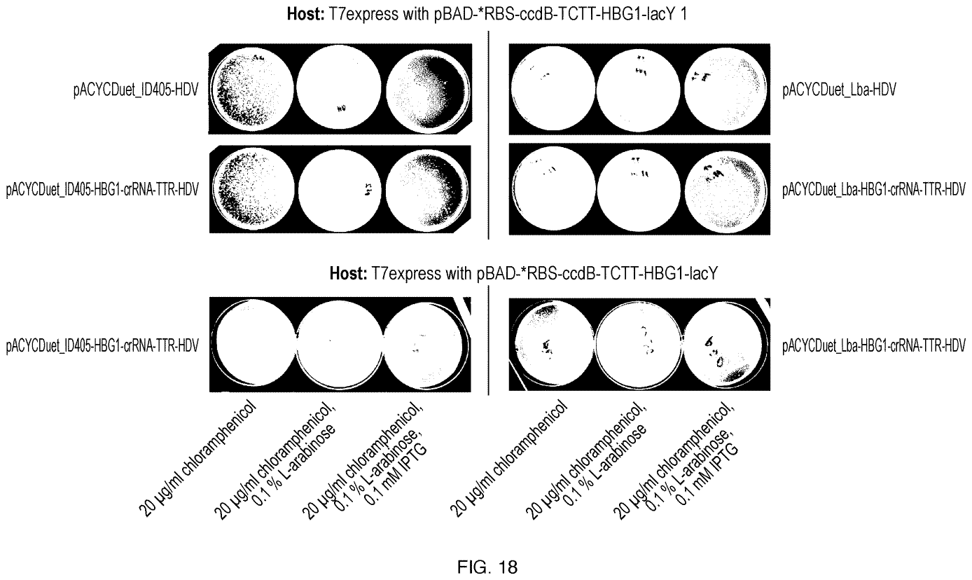

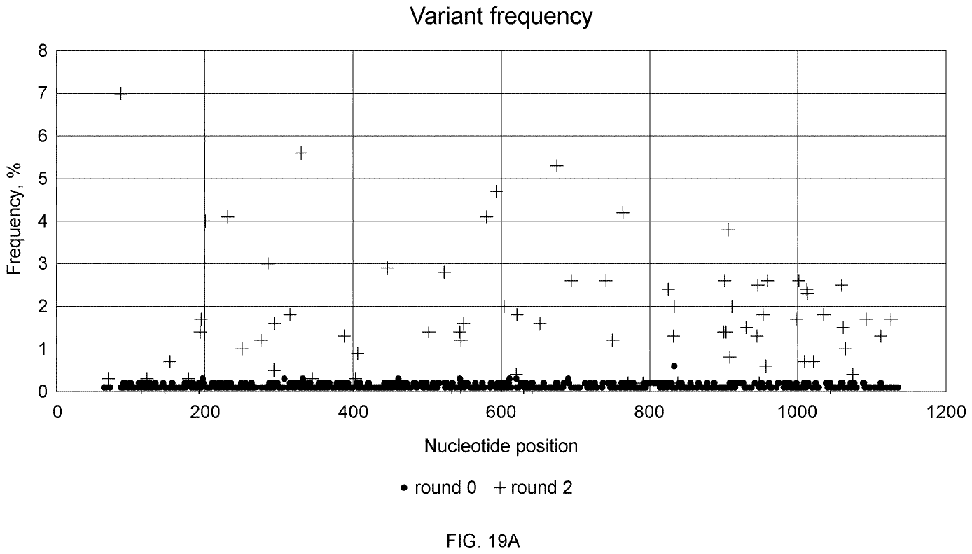

Plates were incubated overnight at 37° C. before inspecting bacterial growth. FIG. 18 shows that ID405 cuts toxin plasmid with target downstream TCTT PAM only in the presence of IPTG (i.e. higher protein concentration), while target downstream TTGG PAM is not cut. This shows that TCTT and TTGG can be considered as intermediate, and poor preference PAM sequences, respectively, and that can create a variable selection pressure for ID405 variants. Therefore, library selections with TCTT PAM were performed without IPTG, while library selections with TTGG PAM were performed either without IPTG or in the presence of 0.1 mM IPTG.

The target for all mutagenesis reactions was amplified from pACYCDuet_ID405-crRNA-TTR-HDV using primer pair CZ-1824/CZ-4750. Q5 2× MasterMix (New England Biolabs) was used for the PCR and the reaction set up using 100 ng plasmid as template and 0.5 μM of each primer in a final volume of 50 μL. The cycling conditions used were: 98° C. for 30 s, 30 cycles of 98° C. for 10 s, 70° C. for 10 s, 72° C. for 1 min, and final extension at 72° C. for 2 min. The amplicon was purified using GeneJET PCR Purification Kit according to manufacturer's recommendations. The number of total reactions were dependent on the required final quantity of the fragment required per mutagenesis reaction.

N-terminus library with random mutations was created by amplifying wild-type target gene sequence with error-prone GeneMorph II random mutagenesis kit (Agilent) using primer pair CZ-5248/CZ-5239. 50 μL reaction contained 5 μL 5× reaction buffer, 1 μL 40 mM dNTP mix, 2.5 ng/μL of each primer, 2 μg target, 1 μL Mutazyme II DNA polymerase. The cycling conditions used were: 95° C. for 2 min, 30 cycles of 95° C. for 30 s, 58° C. for 30 s, 72° C. for 1 min 30 s, and final extension at 72° C. for 10 min. The PCR product was size-selected and recovered from a 1% agarose gel using Monarch® DNA Gel Extraction Kit (New England Biolabs). The purified fragment along with pACYCDuet_ID405-crRNA-TTR-HDV plasmid was digested using FD Ncol and FD BamHI (Thermo fisher Scientific) in a total volume of 20 μL that contained 1 μg of DNA, 2 μL FD buffer, 0.5 μL each enzyme, water up to 20 μL. The prepared reactions were incubated at 37° C. for 1 h. The cut plasmid was size-selected and recovered from a 1% agarose gel using Monarch® DNA Gel Extraction Kit (New England Biolabs), while the fragment was purified with Monarch® PCR & DNA Cleanup Kit. The plasmid and insert were ligated using 1 μL T4 ligase (New England Biolabs), 2 μL 10× T4 ligase buffer, 50 ng plasmid, 25 ng insert, in a total volume of 20 μL. The reaction conditions used were 16° C. for 16 h, 65° C. for 10 minutes. Reactions were scaled 100-fold to increase the size of resulting library. Ligations were purified and concentrated using Monarch® PCR & DNA Cleanup Kit (New England Biolabs) into 15 μL.

Libraries were delivered into NEB® 10-beta Electrocompetent E. coli (New England Biolabs) by MicroPulser Electroporator (biorad) using cold 0.1 cm electrode gap cuvettes and under 1.8 kV voltage. Transformed cells were pooled and recovered for 1 h and 10 min with vigorous shaking at 37° C. After which 100 μL of 10/100/1000/10000-fold dilutions were plated on LB medium containing 20 μg/mL chloramphenicol plates for enumeration, while the remaining transformation mix with library was amplified by adding 10× volume of fresh LB medium containing 20 μg/mL chloramphenicol and continuing shaking for additional 11 hours at 37° C. followed by incubation at 4° C. until collection by centrifugation at 2500×g for 15 min. The plasmid library was extracted using ZymoPURE II Plasmid Midiprep Kit (Zymo).

All other libraries were created by Golden Gate assembly. Firstly, new ID405 plasmids were constructed by replacing C-terminus, N-2, C-2 regions in pACYCDuet_ID405-crRNA-TTR-HDV with BsaI restriction sites by amplifying plasmid with primer pairs CZ-5240/CZ-5249, CZ-5669/CZ-5670, CZ-5673/CZ-5674, respectively. The amplicons were ligated onto themselves using KLD Enzyme Mix (New England Biolabs) and transformed into NEB® 5-alpha Competent E. coli (New England Biolabs) according to manufacturer's recommendations. The plasmids were extracted using ZymoPURE II Plasmid Midiprep Kit (Zymo). Fragments with random mutations were created by amplifying wild-type target gene sequence with error-prone GeneMorph II random mutagenesis kit using primer pairs CZ-5250/CZ-5251, CZ-5672/CZ-5760, CZ-5675/CZ-5676 for C-terminus, N-2, C-2 regions, respectively. Composition and cycling conditions of PCR reactions were the same as described above, except target quantity in C-terminus, N-2, C-2 reactions were 2 μg, 100 ng, 10 ng, respectively. This was determined empirically after the transformation of constructed library into E. coli, by nanopore sequencing of 10 random plasmids or PCR fragments, amplified from randomly picked colonies using primer pair CZ-1824/CZ-4750, to achieve ~2-3 average nucleotide mutations per sequence. The PCR products were size-selected and recovered from a 1% agarose gel using Monarch® DNA Gel Extraction Kit (New England Biolabs). The assembly of libraries were conducted as follows: 20 μL reaction contained 100 ng plasmid DNA, insert quantity were varied to keep molar ratio of 1:3 plasmid to insert, 2 μL T4 ligation buffer, 0.5 μl T4 ligase (New England Biolabs), 0.5 μl BsaI-HFv2 (New England Biolabs), H₂O up-to 20 μL. The cycling conditions used were: 50 cycles of 37° C. for 5 min of 16° C. for 5 min, 1 cycle of 50° C. for 5 min, 1 cycle of 65° C. for 5 min. Reactions for the final libraries were scaled 100-fold to increase the size of the resulting libraries. The final reactions were pooled, purified, transformed, and amplified as described above. The summary of resulting libraries is given in Table 15C.

TABLE 15C
Summary of libraries. Average mutation rate was determined
by nanopore sequencing of 10 random plasmids or PCR fragments,
amplified from randomly picked colonies using primer pair CZ-
1824/CZ-4750 after transformation into NEB ® 10-beta
Electrocompetent E. coli (New England Biolabs)

| | N-terminus | N-2 | C-2 | C-terminus |
|---|---|---|---|---|
| Mutagenized | 1072 | 698 | 420 | 1310 |
| fragment length | | | | |
| Average | 2 | 3.2 | 2.1 | 1.8 |
| mutation | | | | |
| Library size, | 4.65E+07 | 8.50E+07 | 1.36E+07 | 2.40E+08 |
| (estimated from | | | | |
| the enumeration | | | | |
| of colonies) | | | | |

ID405 mutant libraries were enriched for enhanced activity over 2 to 5 rounds of selections. Approximately 5 to 10-fold larger number of E. coli cells containing pBAD-*RBS-ccdB-TCTT-HBG1-lacY or pBAD-*RBS-ccdB-TTGG-HBG1-lacY than estimated plasmid library was transformed by electroporation of each library. Bacteria were recovered in SOC medium for 1 h and 30 min with vigorous shaking at 37° C. Then, bacteria were plated on LB medium containing 20 μg/mL chloramphenicol and 0.1% L-arabinose. The surviving cells were collected by adding 4 mL of sterile LB medium and scraping them with cell spreader. Plasmids from pooled fractions were extracted by Monarch® Plasmid Miniprep Kit (New England Biolabs) and used for the next round of selection. 2 rounds of selection were performed with TCTT PAM, 5 rounds of selection were performed with TTGG PAM. Additionally, selection with TTGG PAM was performed using either medium without IPTG or in the presence of 0.1 mM IPTG.

### Deep Sequencing of the Libraries

The mutagenized regions of libraries were amplified using primer pairs CZ-5165/CZ-5167, CZ-5672/CZ-5760, CZ-5675/CZ-5676, CZ-5170/CZ-5171 for N-terminus, N-2, C-2, C-terminus libraries, respectively. Q5 2× MasterMix (New England Biolabs) was used for the PCR and the reaction set up using 300 ng plasmid as template and 0.5 μM of each primer in a final volume of 60 μL over 2 vials. The cycling conditions used were 98° C. for 30 s, 16 cycles of 98° C. for 10 s, 66° C. for 10 s, 72° C. for 1 min, and final extension at 72° C. for 2 min. After PCR, 1 μL of DpnI was added to the samples and incubated at 37° C. for 30 min. Illumina libraries were prepared with NEBNext® Ultra™ II FS DNA Library Prep with Sample Purification Beads kit (New England Biolabs), using the protocol for inputs ≤100 ng and fragmentation time of 20 min.

Sample libraries were checked using Tapestation D1000 DNA ScreenTape Analysis kit (Agilent), quantified with Qubit fluorometer (Thermo Fisher Scientific) and pooled in equimolar ratio. Final library pool was quantified via qPCR using NEBNext® Ultra™ II DNA Library Prep Kit for Illumina® (New England Biolabs). Paired-end sequencing (2×150 cycles) was performed on Illumina MiSeq system with 1% PhiX spike-in. Sequencing data analysis was performed using Geneious Prime (version 2024.0.4, GraphPad Software LLC d.b.a Geneious). Reads were merged, trimmed and filtered using BBDuk and mapped to the reference sequence of wild type ID405 using Bowtie2. Geneious variant finder was used to call SNPs with detection limit set to 0.001%. A threshold of 5% for the frequency of SNP was established when determining enriched sequences/positions.

### Analysis of Sequencing Results

The results of the deep sequencing are summarized in Table 15D. The enrichment of more than 5% of variant frequency was observed only in the 5th round of libraries where TTGG PAM was used, while with TCTT PAM we observed enrichment after the 2nd round of iteration. Note, that 2 rounds of enrichment were chosen for the latter. An unintentional selection of plasmids with deletion in the region encoding crRNA was observed, which resulted in PAM shift and reinstation of WT PAM. Hence, resulting in no selective pressure for enrichment of mutant ID405 sequences.

TABLE 15D
Summary of sequencing results. R, denotes round
of iteration, TCTT and TTGG denote ccdB plasmid
used in the selection, N and C denote library

| | | | | |
|---|---|---|---|---|
| | | | | Max |
| | | | | variant |
| | | Total reads | | fre- |
| | | (paired end, | Mapped | quency, |
| No. | Sample ID | merged) | reads | % |

N-terminus libraries

| | | | | |
|---|---|---|---|---|
| 1 | R0_N | 935 070 | 670 405 | 0.6 |
| 2 | R2_N_TCTT | 872 554 | 789 661 | 7 |
| 3 | R3_N_TTGG | 1 014 312 | 890 344 | 3.3 |
| 4 | R5_N_TTGG | 936 034 | 738 434 | 20 |
| 11 | R3_N_IPTG_TTGG | 934 055 | 798 547 | 2.1 |
| 12 | R5_N_IPTG_TTGG | 906 941 | 366 749 | 15.2 |

C-terminus libraries

| | | | | |
|---|---|---|---|---|
| 5 | R0_C | 973 675 | 799 807 | 1.4 |
| 6 | R2_C_TCTT | 1 301 019 | 1 188 979 | 1.5 |
| 7 | R3_C_TTGG | 949 438 | 873 557 | 1.3 |
| 8 | R5_C_TTGG | 781 297 | 205 167 | 8.3 |
| 13 | R3_C_IPTG_TTGG | 940 320 | 873 201 | 2.3 |
| 14 | R5_C_IPTG_TTGG | 732 500 | 32 591 | 43.5 |

N-2 libraries

| | | | |
|---|---|---|---|
| R1_0 | 843 707 | 735 572 | 0.5 |
| R1_TCTT_R2 | 706 586 | 684 189 | 52.7 |
| R1_TTGG_R3 | 740 156 | 713 414 | 15.4 |
| R1_TTGG_R5 | 679 459 | 603 365 | 70.6 |
| R1_TTGG_R3_IPTG | 696 137 | 664 003 | 4.3 |
| R1_TTGG_R5_IPTG | 701 764 | 598 860 | 27.4 |

C-2 libraries

| | | | |
|---|---|---|---|
| R2_0 | 762 972 | 720 095 | 0.9 |
| R2_TCTT_R2 | 894 432 | 636 686 | 6.7 |
| R2_TTGG_R3 | 786 332 | 709 136 | 7.1 |
| R2_TTGG_R5 | 864 312 | 665 958 | 26.6 |
| R2_TTGG_R3_IPTG | 826 132 | 789 501 | 4.4 |
| R2_TTGG_R5_IPTG | 945 107 | 688 684 | 34.1 |

C-2 libraries Rep

| | | | |
|---|---|---|---|
| R1_TTGG_R3_2 | 829 990 | 587 216 | 1.5 |
| R1_TTGG_R4_2 | 871 566 | 663 469 | 3.6 |
| R1_TTGG_R3_IPTG_2 | 799 043 | 301 339 | 9.8 |
| R1_TTGG_R4_IPTG_2 | 682 707 | 33 418 | 11.5 |

### N-Terminus Libraries

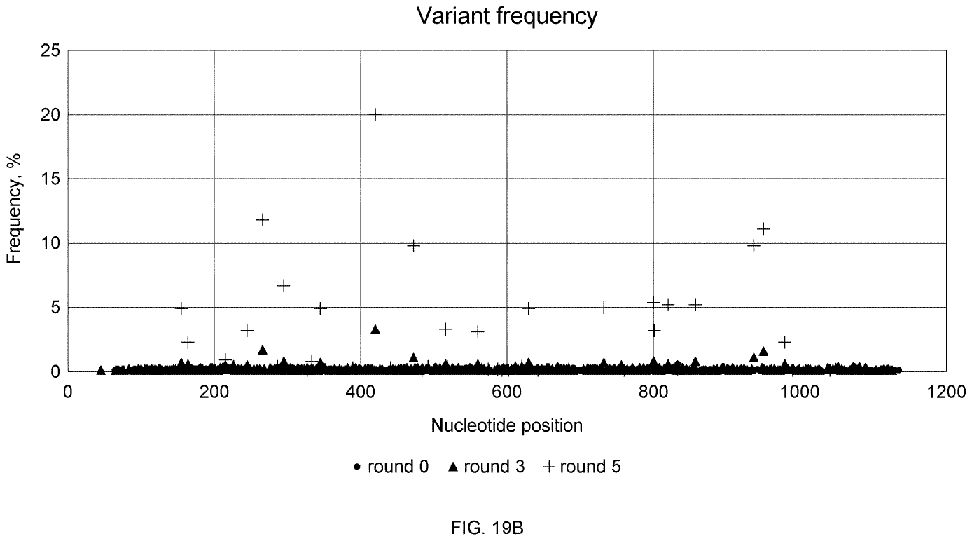

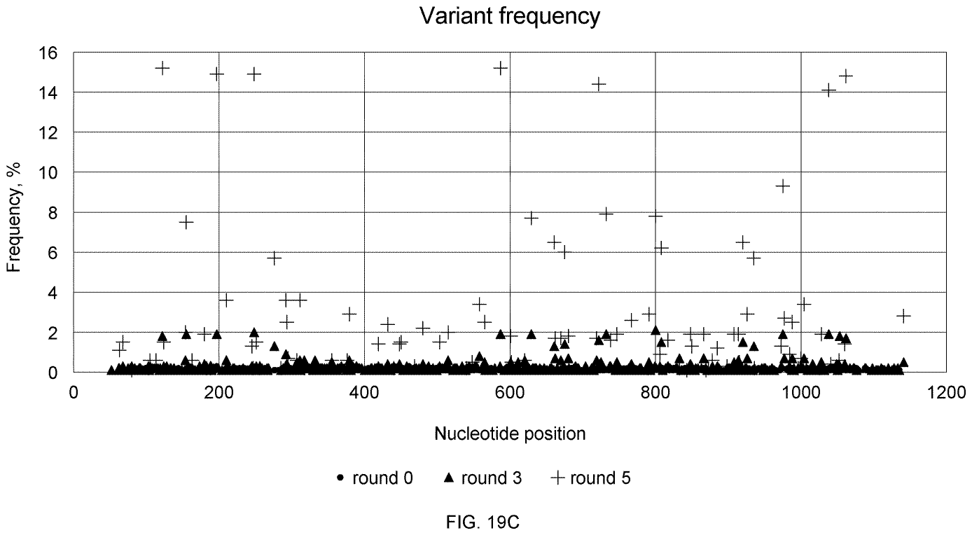

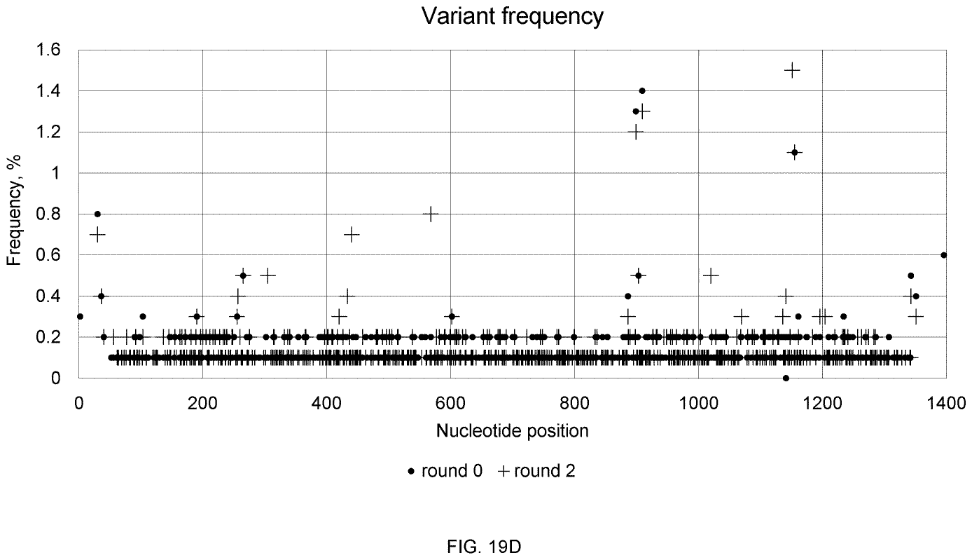

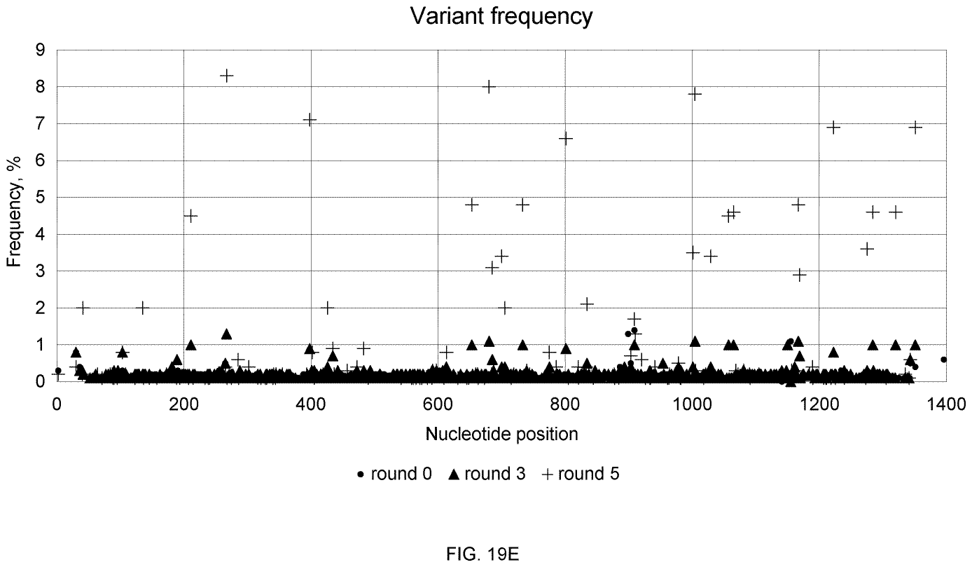

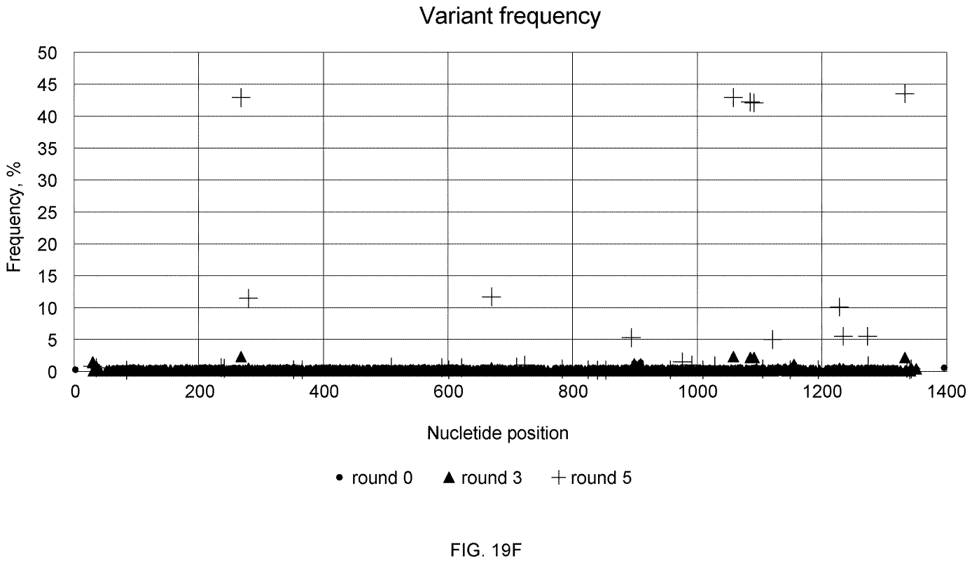

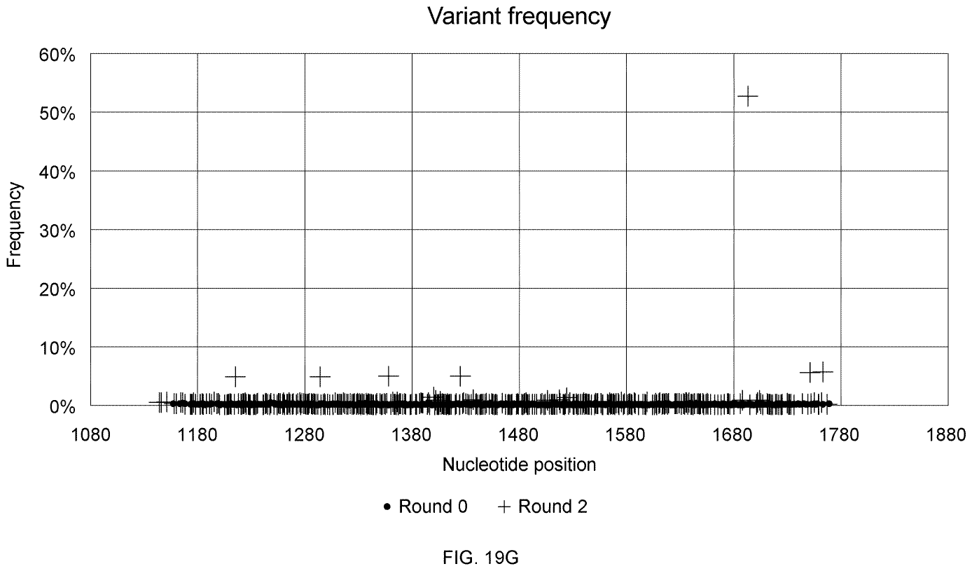

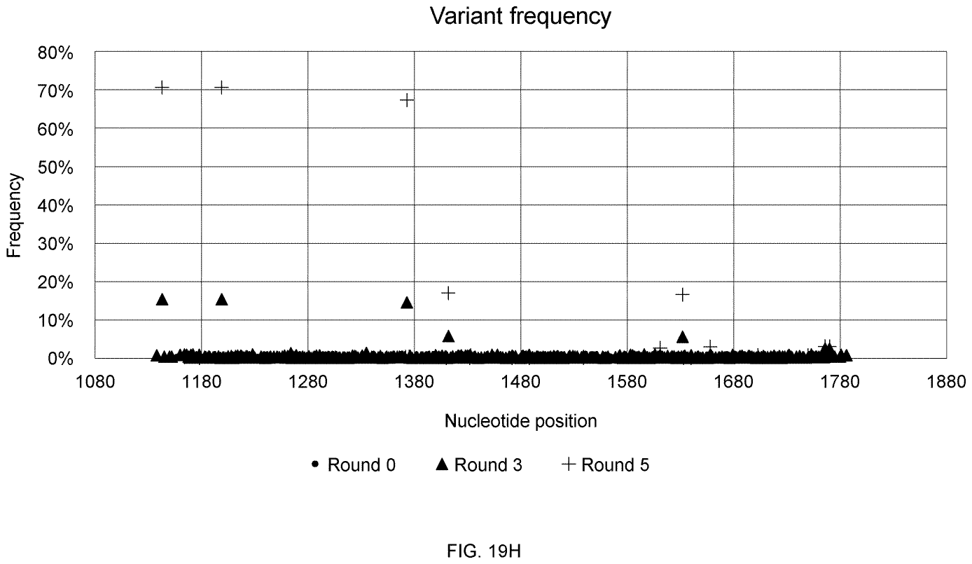

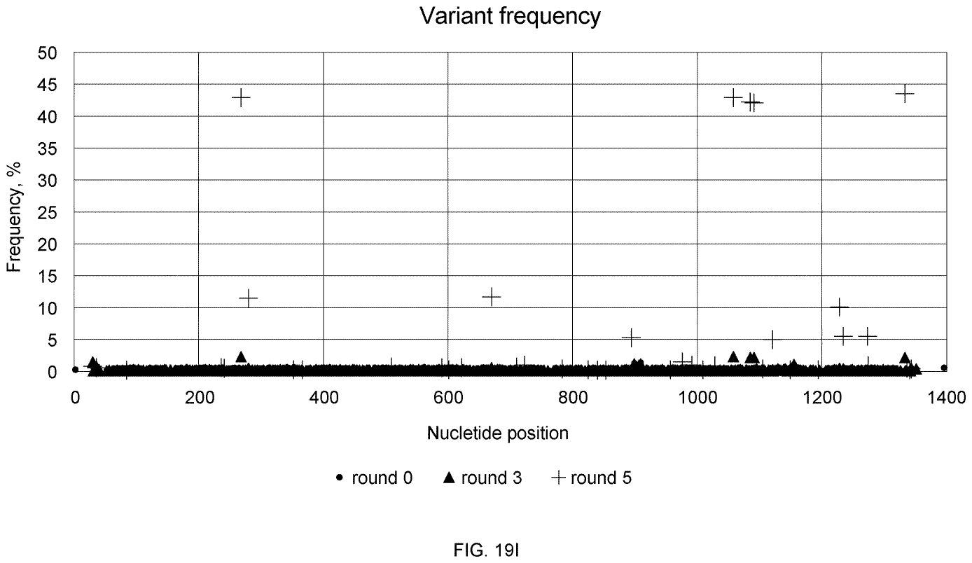

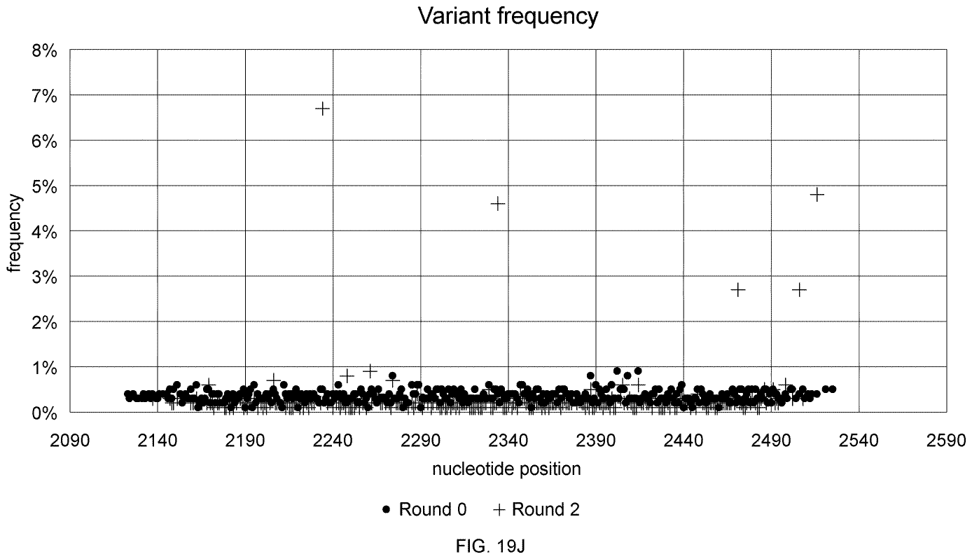

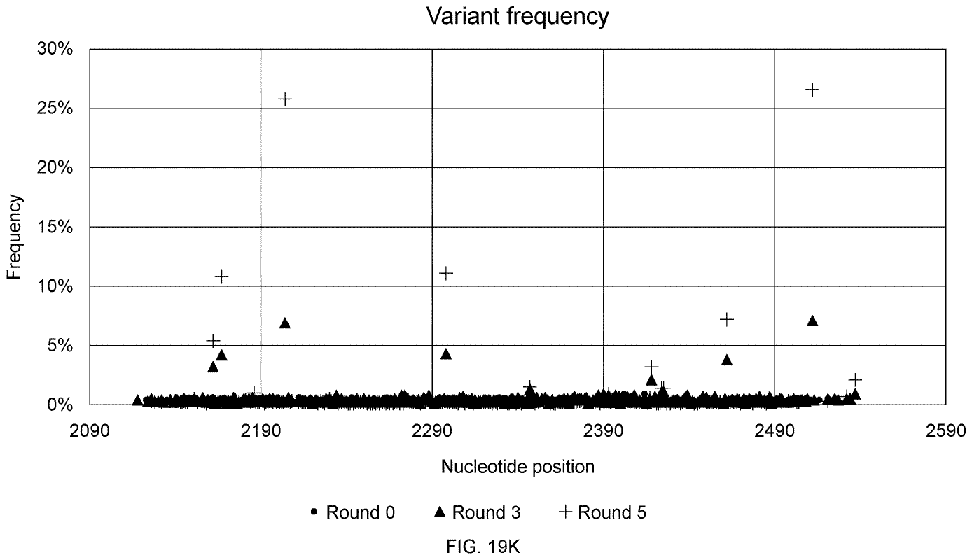

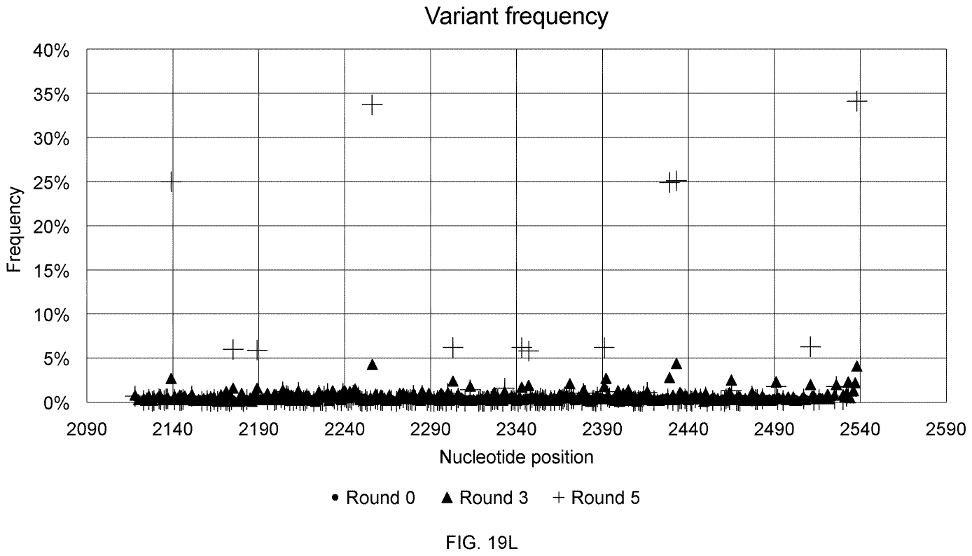

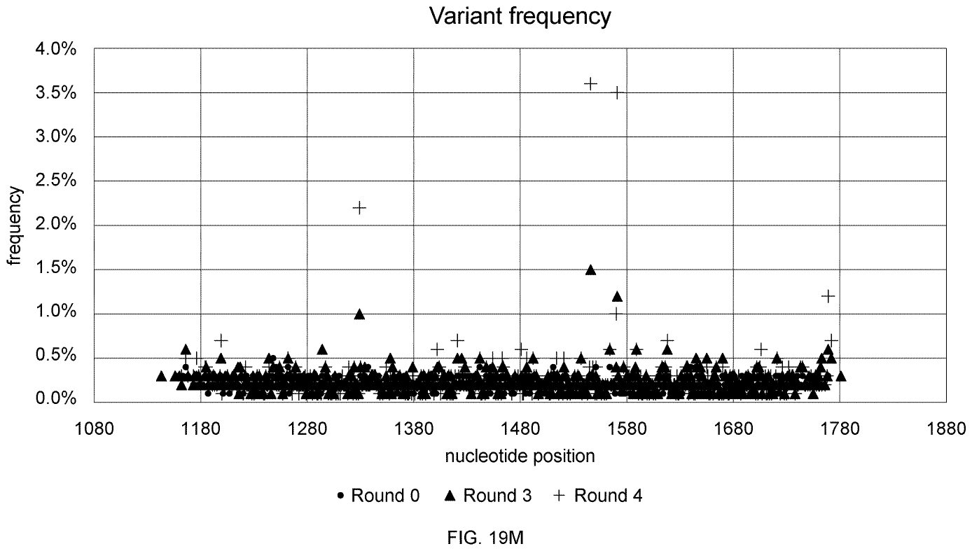

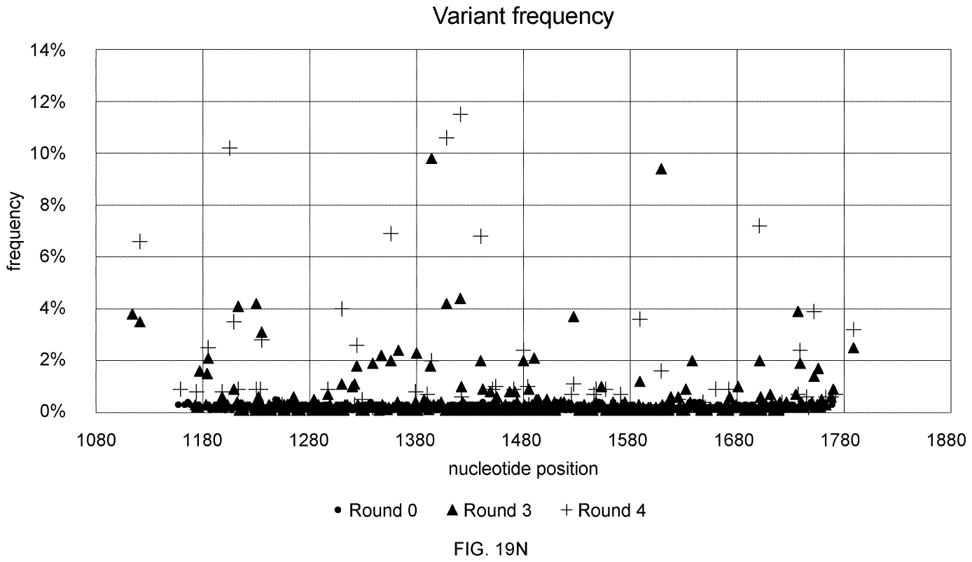

82 variants in round 2 were detected in the N-terminus library under the selection with TCTT PAM (FIG. 19A). From these, three variants exhibited frequency above or equal 5% (Table 15E). The protein (SEQ ID NOs: 96-126) and coding DNA sequences (SEQ ID NOs: 127-157) of the selected ID405 type V variants with mutations in N terminus are presented in the SEQ Listing Appendix. Bold letters in the SEQ indicates the mutant amino acid.

TABLE 15E
Substitutions with frequency above or equal 5% in N-
terminus library under the selection against TCTT PAM

| CDS | | Codon | Amino Acid Change | Variant | |
|---|---|---|---|---|---|
| Position | Change | Change | (relative to ID405) | Frequency, % | SEQ ID NO: |
| 47 | G −> C | AGC −> ACC | S16T | 7 | SEQ ID NO: 127 |
| 290 | T −> C | ATC −> ACC | I97T | 5.6 | SEQ ID NO: 128 |
| 635 | G −> T | CGA −> CTA | R212L | 5.3 | SEQ ID NO: 129 |

In N-terminus library under the selection with TTGG PAM, we detected 618 variants in round 3 and 42 variants in round 5 (FIG. 19B). From these, five variants exhibited frequency above or equal to 5% (Table 15F).

TABLE 15F
Substitutions with frequency above or equal 5% in N-
terminus library under the selection against TTGG PAM

| CDS | | Codon | Amino Acid Change | Variant | |
|---|---|---|---|---|---|
| Position | Change | Change | (relative to ID405) | Frequency, % | SEQ ID NO: |
| 380 | A −> G | AAA −> AGA | K127R | 20 | SEQ ID NO: 130 |
| 226 | A −> G | AAC −> GAC | N76D | 11.8 | SEQ ID NO: 131 |
| 910 | C −> A | CGT −> AGT | R304S | 11.1 | SEQ ID NO: 132 |
| 760 | G −> A | GTG −> ATG | V254M | 5.4 | SEQ ID NO: 133 |
| 692 | A −> T | GAT −> GTT | D231V | 5 | SEQ ID NO: 134 |

In N-terminus library under the selection with TTGG PAM under the presence of 0.1 mM IPTG, 572 variants in round 3 and 109 variants in round 5 (FIG. 19C) were detected. From these, twelve variants exhibited frequency above or equal to 5% (Table 15G).

TABLE 15G
Substitutions with frequency above or equal 5% in N-terminus library under
the selection against TTGG PAM under the presence of 0.1 mM IPTG

| CDS | | Codon | Amino | Variant | |
|---|---|---|---|---|---|
| Position | Change | Change | Acid Change | Frequency, % | SEQ ID NO: |
| 82 | A −> T | ATC −> TTC | I28F | 15.2 | SEQ ID NO: 135 |
| 547 | G −> A | GCA −> ACA | A183T | 15.2 | SEQ ID NO: 136 |
| 1022 | A −> T | GAT −> GTT | D341V | 14.8 | SEQ ID NO: 137 |
| 935 | T −> A | GTG −> GAG | V312E | 9.3 | SEQ ID NO: 138 |
| 692 | A −> T | GAT −> GTT | D231V | 7.9 | SEQ ID NO: 134 |
| 760 | G −> A | GTG −> ATG | V254M | 7.8 | SEQ ID NO: 133 |
| 589 | T −> G | TTT −> GTT | F197V | 7.7 | SEQ ID NO: 139 |
| 621 | A −> T | AAA −> AAT | K207N | 6.5 | SEQ ID NO: 140 |
| 880 | G −> A | GTG −> ATG | V294M | 6.5 | SEQ ID NO: 141 |
| 635 | G −> A | CGA −> CAA | R212Q | 6 | SEQ ID NO: 142 |
| 236 | T −> C | TTT −> TCT | F79S | 5.7 | SEQ ID NO: 143 |
| 895 | C −> G | CAG −> GAG | Q299E | 5.7 | SEQ ID NO: 144 |

### C-Terminus Libraries

In the C-terminus library under the selection with TCTT PAM, 681 variants in round 2 were detected (FIG. 19D). From these, 0 variants exhibited frequency above or equal to 5%. The protein (SEQ ID NO. 96-126) and coding DNA sequences (SEQ ID NO. 127-157) of the selected ID405 Cas12a variants with mutations in C terminus are presented in the SEQ Listing Appendix.

In C-terminus library under the selection with TTGG PAM, we detected 801 variants in round 3 and 165 variants in round 5 (FIG. 19E). From these, three variants exhibited frequency above or equal to 5% (Table 15H).

TABLE 15H
Substitutions with frequency above or equal 5% in C-
terminus library under the selection against TTGG PAM

| CDS | | Codon | Amino Acid | Variant | |
|---|---|---|---|---|---|
| Position | Change | Change | Change | Frequency, % | SEQ ID NO: |
| 2825 | G −> A | GTT −> ATT | V929I | 8.3 | SEQ ID NO: 145 |
| 3780 | A −> G | GAT −> GGT | D1247G | 6.9 | SEQ ID NO: 146 |
| 3359 | A −> T | ACC −> TCC | T1107S | 6.6 | SEQ ID NO: 147 |

In C-terminus library under the selection with TTGG PAM under the presence of 0.1 mM IPTG, we detected 1001 variants in round 3 and 41 variants in round 5 (FIG. 19F). From these, 10 variants were with frequency above or equal 5% (Table 15I).

TABLE 15I
Substitutions with frequency above or equal 5% in C-terminus library
under the selection against TTGG PAM under presence of 0.1 mM IPTG

| CDS | | Codon | Amino | Variant | |
|---|---|---|---|---|---|
| Position | Change | Change | Acid Change | Frequency, % | SEQ ID NO: |
| 3851 | A −> G | AAA −> AGA | K1284R | 43.5 | SEQ ID NO: 148 |
| 2786 | T −> A | GTT −> GAT | V929D | 42.9 | SEQ ID NO: 149 |
| 3576 | C −> G | GAC −> GAG | D1192E | 42.9 | SEQ ID NO: 150 |
| 3609 | G −> T | CAG −> CAT | Q1203H | 42.1 | SEQ ID NO: 151 |
| 3188 | T −> A | ATC −> AAC | I1063N | 11.7 | SEQ ID NO: 152 |
| 2798 | A −> T | AAG −> ATG | K933M | 11.5 | SEQ ID NO: 153 |
| 3746 | A −> G | AAT −> AGT | N1249S | 10.1 | SEQ ID NO: 154 |
| 3752 | C −> T | GCG −> GTG | A1251V | 5.5 | SEQ ID NO: 155 |
| 3791 | A −> T | AAT −> ATT | N1264I | 5.5 | SEQ ID NO: 156 |
| 3412 | C −> A | CGT −> AGT | R1138S | 5.3 | SEQ ID NO: 157 |

In C-terminus library under the selection with TTGG PAM under the presence of 0.1 mM IPTG resulted in the highest enrichment frequencies of ID405 variants. It was then evaluated whether these mutations are linked (meaning they are found on the same sequence) by randomly sequencing several Round 5 transformants. It was determined that V929D; D1192E; Q1203H; K1284R mutations are found on the same sequence.

### N-2 Libraries

In the N-2 library under selection with TCTT PAM, 476 variants were detected in round 2 (FIG. 19G). From these, five variants were detected with frequency above or equal 5% (Table 15J). The protein (SEQ ID NOs: 754-781) and coding DNA sequences (SEQ ID NOs: 793-820) of the selected ID405 Cas12a variants with mutations are presented in the SEQ Listing Appendix.

TABLE 15J
Substitutions with frequency above or equal 5% in
N-2 library under the selection against TCTT PAM

| CDS | | Codon | Amino | Variant | |
|---|---|---|---|---|---|
| Position | Change | Change | Acid Change | Frequency, % | SEQ ID NO: |
| 1693 | C −> A | CAA −> AAA | Q565K | 52.70% | SEQ ID NO: 793 |
| 1763 | A −> T | AAC −> ATC | N588I | 5.70% | SEQ ID NO: 794 |
| 1751 | T −> G | ATT −> AGT | I584S | 5.60% | SEQ ID NO: 795 |
| 1358 | T −> A | GTG −> GAG | V453E | 5.00% | SEQ ID NO: 796 |
| 1425 | A −> T | CAA −> CAT | Q475H | 5.00% | SEQ ID NO: 797 |

In the N-2 library under selection with TTGG PAM, 496 variants were detected in round 3 and 27 variants in round 5 (FIG. 19H). From these, three variants were detected with frequency above or equal 5% (Table 15K). Note that during the selection of this library, only 3.06E+07 E. coli cells were transformed in round, i.e. library was underrepresented as initial library size was ~8.50E+07 plasmids (Table 15C).

TABLE 15K
Substitutions with frequency above or equal 5% in
N-2 library under the selection against TTGG PAM

| CDS | | Codon | Amino | Variant |
|---|---|---|---|---|
| Position | Change | Change | Acid Change | Frequency, % |
| 1199 | A −> G | AAT −> AGT | N400S | 70.60% |
| 1412 | T −> A | ATT −> AAT | I471N | 17.00% |
| 1632 | G −> T | GAG −> GAT | E544D | 16.60% |

In the N-2 library under selection with TTGG PAM under the presence of 0.1 mM IPTG, 549 variants were detected in round 3 and 155 variants in round 5 (FIG. 19I). From these, 15 variants were detected with frequency above or equal 5% (Table 15L). Note that during the selection of this library, only 3.06E+07 E. coli cells were transformed in the round, i.e. the library was underrepresented as the initial library size was ~8.50E+07 plasmids (Table 15C).

TABLE 15L
Substitutions with frequency above or equal 5% in N-2 library under
the selection against TTGG PAM under presence of 0.1 mM IPTG

| | | | Amino | Variant | |
|---|---|---|---|---|---|
| CDS Position | Change | Codon Change | Acid Change | Frequency, % | SEQ ID NOs |
| 1486 | C −> A | CAC −> AAC | H496N | 27.40% | SEQ ID NO: 801 |
| 1600 | G −> T | GTC −> TTC | V534F | 27.10% | SEQ ID NO: 802 |
| 1730 | A −> G | GAC −> GGC | D577G | 25.60% | SEQ ID NO: 803 |
| 1202 | A −> T | AAG −> ATG | K401M | 24.30% | SEQ ID NO: 804 |
| 1645 | A −> G | AAG −> GAG | K549E | 24.00% | SEQ ID NO: 805 |
| 1736 | A −> T | AAA −> ATA | K579I | 23.50% | SEQ ID NO: 806 |
| 1525 | A −> G | AAA −> GAA | K509E | 23.00% | SEQ ID NO: 807 |
| 1784 | A −> C | AAG −> ACG | K595T | 19.10% | SEQ ID NO: 808 |
| 1502 | T −> A | GTC −> GAC | V501D | 18.00% | SEQ ID NO: 809 |
| 1703 | G −> T | AGC −> ATC | S568I | 18.00% | SEQ ID NO: 810 |
| 1409 | A −> T | CAC −> CTC | H470L | 17.50% | SEQ ID NO: 811 |
| 1327 | G −> A | GGA −> AGA | G443R | 7.90% | SEQ ID NO: 812 |
| 1771 | C −> T | CCG −> TCG | P591S | 7.70% | SEQ ID NO: 813 |
| 1547 | A −> C | AAA −> ACA | K516T | 7.50% | SEQ ID NO: 814 |
| 1148 | A −> T | GAG −> GTG | E383V | 7.20% | SEQ ID NO: 815 |

### C-2 Libraries

In the C-2 library under the selection with TCTT PAM, 400 variants were detected in round 2 (FIG. 19J). From these, a single variant was detected with frequency above or equal 5% (Table 15M). The protein (SEQ ID NOs: 782-792) and coding DNA sequences (SEQ ID NOs: 821-831) of the selected ID405 Cas12a variants with mutations are presented in the SEQ Listing Appendix.

TABLE 15M
Substitutions with frequency above or equal 5% in
C-2 library under the selection against TCTT PAM

| CDS | | Codon | Amino | Variant | |
|---|---|---|---|---|---|
| Position | Change | Change | Acid Change | Frequency, % | SEQ ID NOs |
| 2234 | A −> T | AAG −> ATG | K745M | 6.70% | SEQ ID NO: 821 |

In C-2 library under the selection with TTGG PAM, 612 variants were detected in round 3 and 432 variants in round 5 (FIG. 19K). From these, five variants were detected with frequency above or equal 5% (Table 15N).

TABLE 15N
Substitutions with frequency above or equal 5% in
N-2 library under the selection against TTGG PAM

| CDS | | Codon | Amino | Variant | |
|---|---|---|---|---|---|
| Position | Change | Change | Acid Change | Frequency, % | SEQ ID NOs |
| 2512 | A −> T | ACC −> TCC | T838S | 26.60% | SEQ ID NO: 822 |
| 2204 | A −> T | AAC −> ATC | N735I | 25.80% | SEQ ID NO: 823 |
| 2167 | A −> G | ATT −> GTT | I723V | 10.80% | SEQ ID NO: 824 |
| 2462 | A −> T | AAA −> ATA | K821I | 7.20% | SEQ ID NO: 825 |
| 2162 | A −> T | AAG −> ATG | K721M | 5.40% | SEQ ID NO: 826 |

In C-2 library under the selection with TTGG PAM under the presence of 0.1 mM IPTG, 574 variants were detected in round 3 and 275 variants in round 5 (FIG. 19L). From these, 5 variants were with detected frequency above or equal 5% (Table 150)

TABLE 15O
Substitutions with frequency above or equal 5% in C-2 library under
the selection against TTGG PAM under presence of 0.1 mM IPTG

| CDS | | Codon | Amino | Variant | |
|---|---|---|---|---|---|
| Position | Change | Change | Acid Change | Frequency, % | SEQ ID NOs |
| 2429 | C −> T | GCC −> GTC | A810V | 24.90% | SEQ ID NO: 827 |
| 2303 | A −> T | AAG −> ATG | K768M | 6.20% | SEQ ID NO: 828 |
| 2343 | C −> A | AGC −> AGA | S781R | 6.20% | SEQ ID NO: 829 |
| 2175 | A −> T | GAA −> GAT | E725D | 6.00% | SEQ ID NO: 830 |
| 2189 | T −> C | TTG −> TCG | L730S | 5.90% | SEQ ID NO: 831 |

### N-2 Libraries (Repetition).

To fully represent N-2 libraries under the selection with TTGG PAM we repeated transformations using larger number of initial E. coli transformants in round 1 and carrying selections up to round 4.

Under the selection with TTGG PAM, we detected 842 variants in round 3 and 813 variants in round 4 (FIG. 19M). From these, no variants were with frequency above or equal 5%. Under the selection with TTGG PAM under the presence of 0.1 mM IPTG, we detected 490 variants in round 3 and 68 variants in round 4 (FIG. 19N). From these, 5 variants were with frequency above or equal 5% (Table 15P).

TABLE 15P
Substitutions with frequency above or equal 5% in a repeated selection of N-2
library under the selection against TTGG PAM under the presence of 0.1 mM IPTG

| CDS | | Codon | Amino | Variant | |
|---|---|---|---|---|---|
| Position | Change | Change | Acid Change | Frequency, % | SEQ ID NOs |
| 1421 | T −> A | CTG −> CAG | L474Q | 11.50% | SEQ ID NO: 816 |
| 1408 | C −> A | CAC −> AAC | H470N | 10.60% | SEQ ID NO: 817 |
| 1205 | A −> T | GAG −> GTG | E402V | 10.20% | SEQ ID NO: 818 |

Round 3 variants that exceeded 5%
frequency but dropped down in round 5

| | | | | | |
|---|---|---|---|---|---|
| 1394 | A −> G | CAA −> CGA | Q465R | 9.80% | SEQ ID NO: 819 |
| 1609 | T −> A | TTT −> ATT | F537I | 9.40% | SEQ ID NO: 820 |

### Example 16: Method for Engineering Guide RNAs with Modifications at Select Ranked Positions by Computational Means

Type V nuclease-guide RNA co-complexes within the protein data bank (PDB) were examined in order to determine key structural interactions of guide backbone atoms (e.g. O2′ or phosphate hydrogen bonds). Type V nuclease-guide RNA co-complexes considered included AsCas12 (Ternary: 5b43, 5kk5), lbCas12 (Ternary: 5XUS, 5XUT, 5XUU, 5XUZ; Binary, 5ID6, 6NM) and FnCas12 (Ternary: 5MGA, 6I1L, 611K, 5NFV; Binary: 5NG6).

Identification of key interactions within each co-complex were determined by using the protein contacts panel within Molecular Operating Environment (MOE), a computer aided molecular design software developed by Chemical Computing Group (CCG). The AMBER10 force field for proteins and nucleic acids (ff10) was used to calculate energetics over six types of contacts: Hydrogen bonds (Hbond), Metal, Ionic, Arene, Covalent, and Van der Waals distance interactions. The solvation model was R-field, the dialectric was set to 1, and Group II ion and Group VIII parameters were from OPLS-AA. Contacts were calculated for both intrachain (within RNA) and interchain (between RNA and protein) by selecting SetA: ChainA (eg protein) and SetB: Full Complex. The sequence separation was set to 2, the distance threshold was set to 4.5, and no energy threshold was used. Calculations were not aggregated (e.g. Aggregate: None) and the text output was saved and used subsequently for filtering by atom interaction type.

Energetic calculations saved from the MOE contacts panel for each co-complex were filtered by atom interaction type (e.g. O2′ or phosphate/OP) and hydrogen bond energies were summed on a per-nucleotide basis. Summed energies for each guide nucleotide were then averaged across each Type V family member and the resulting scores were used as input for a script to calculate optimal sites for guide backbone modifications.

In brief, the guide modification script uses the per-nucleotide scores calculated from MOE and assumes that making a backbone modification to a given nucleotide atom would completely abolish the native intra- or inter-hydrogen bonding interaction calculated for that atom. Using this assumption, the script iterates combinatorically and scores any potential loss in energetics for guides with one, two, three, etc. modifications, outputting in each case a single guide that best retains maximal interactions.

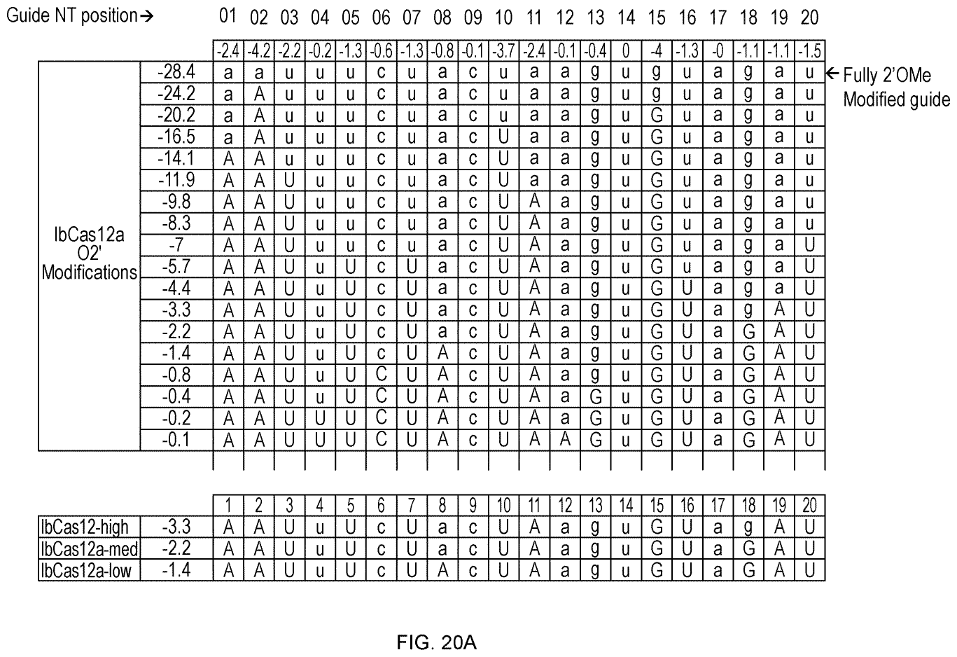

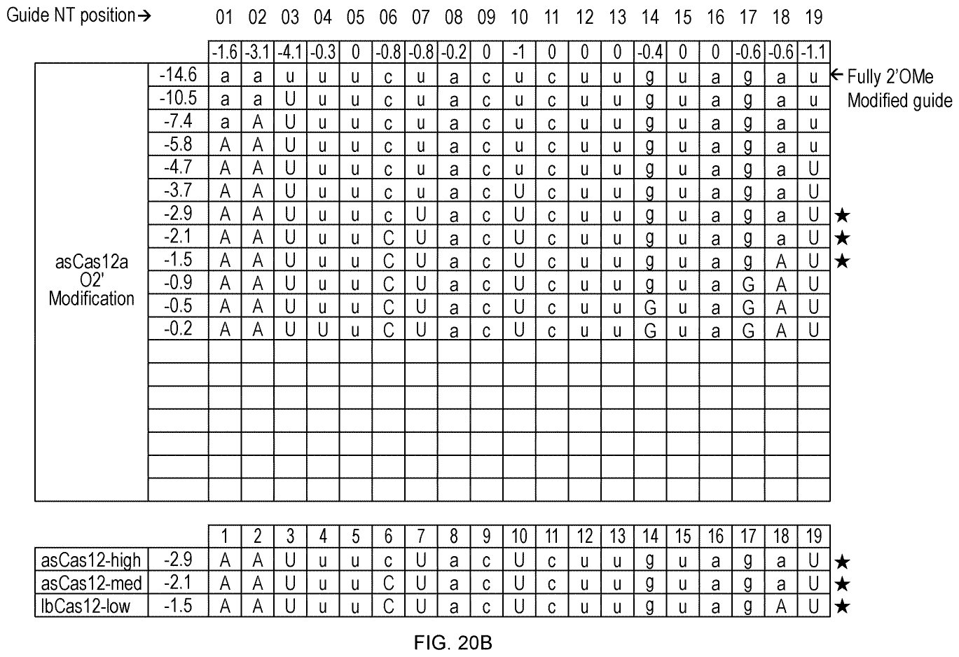

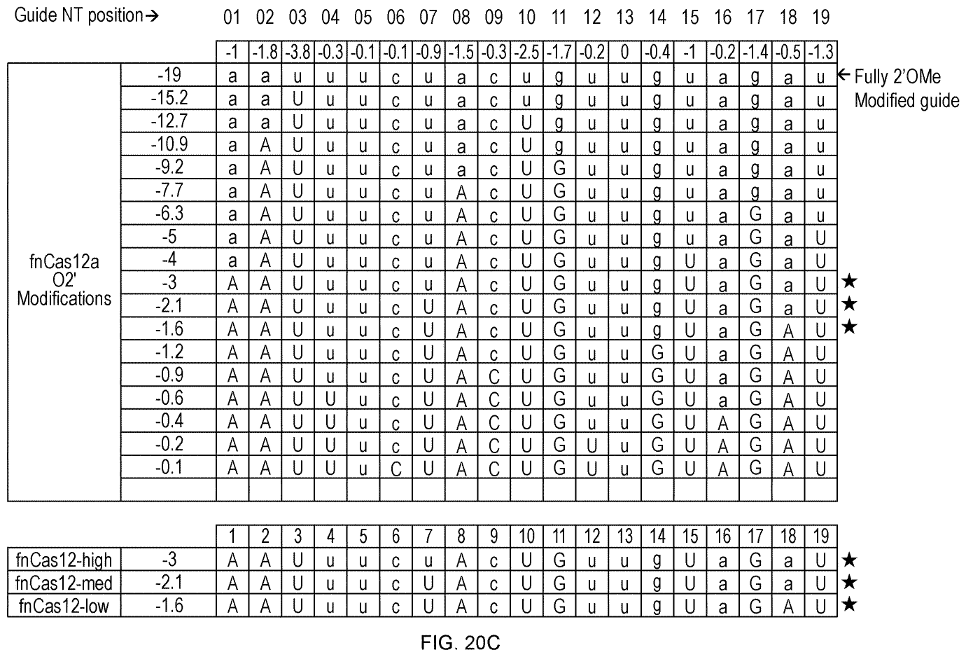

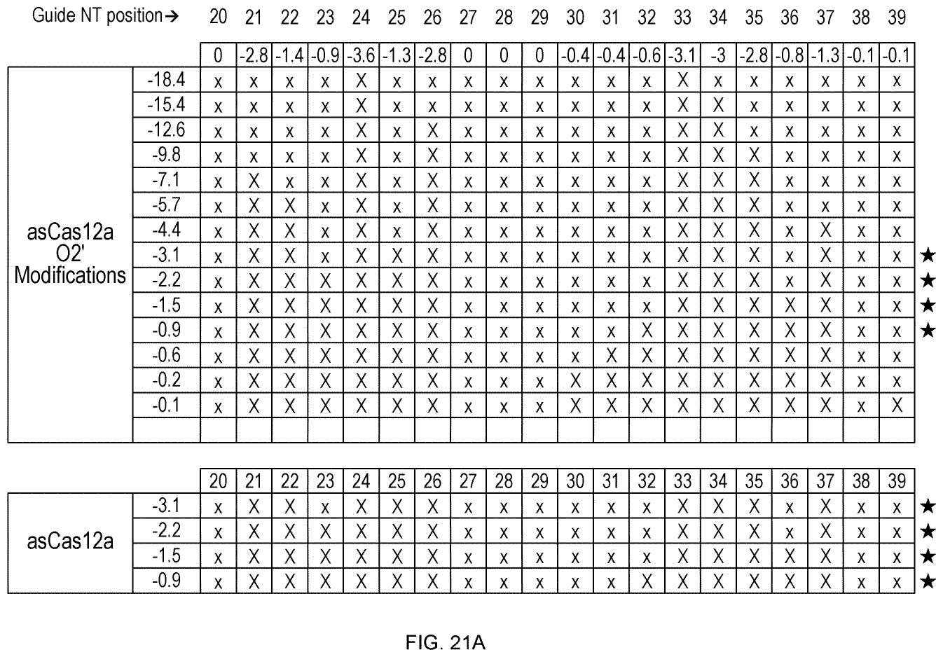

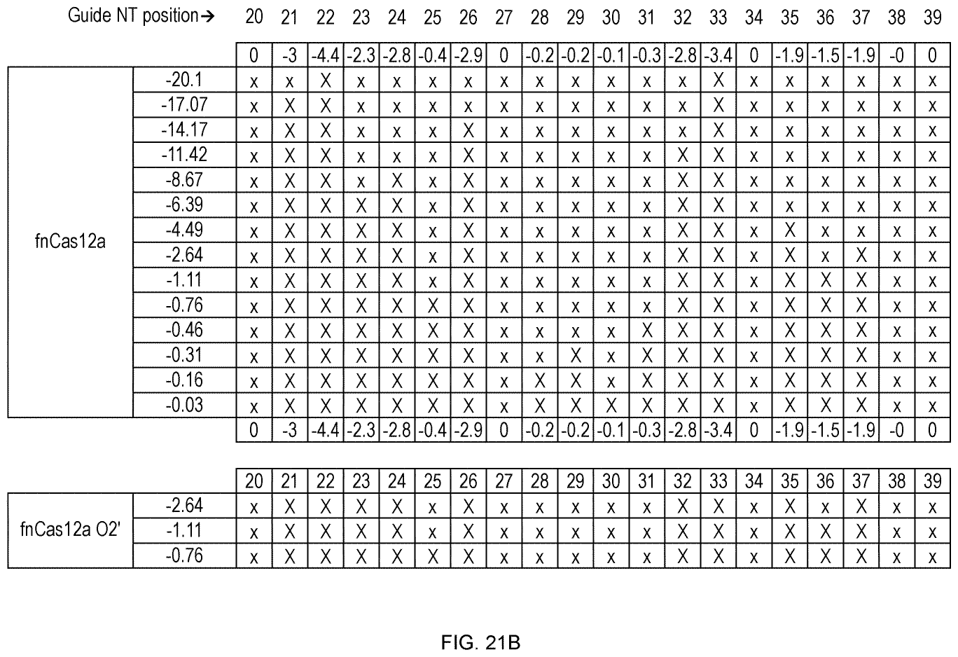

Selection of optimal sites for modification was calculated independently for the scaffold guide regions (FIGS. 20A, 20B, and 20C) and spacer regions (see FIGS. 21A and 21B).

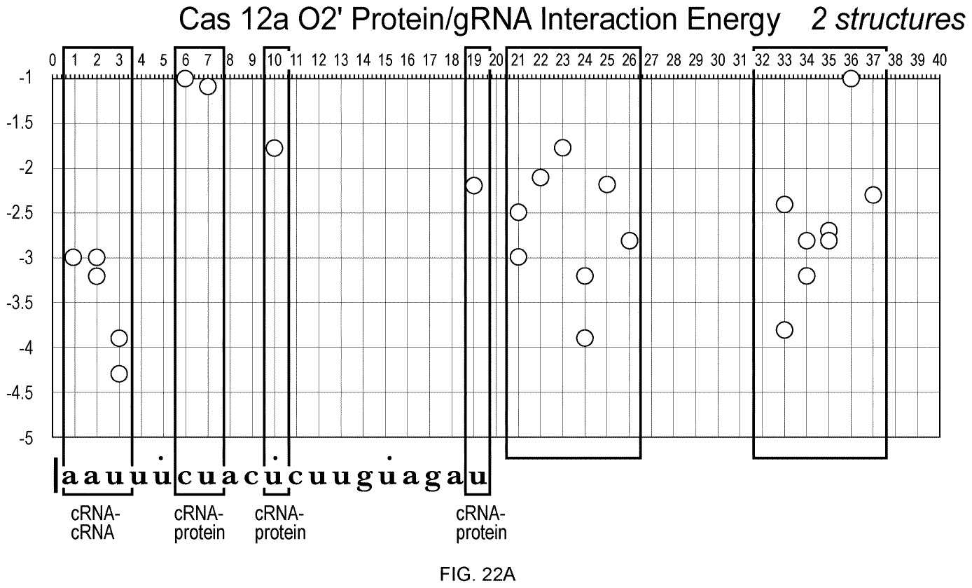

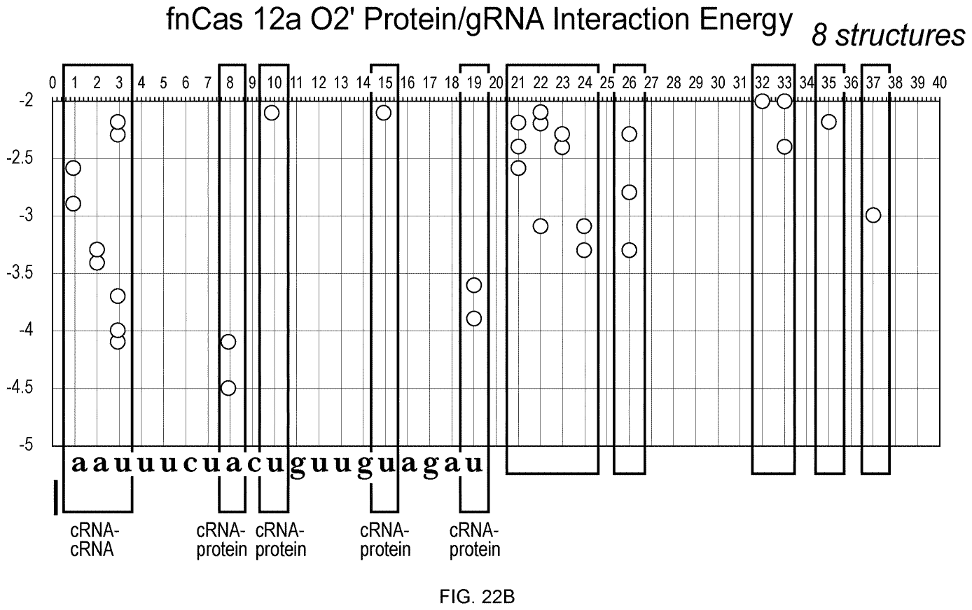

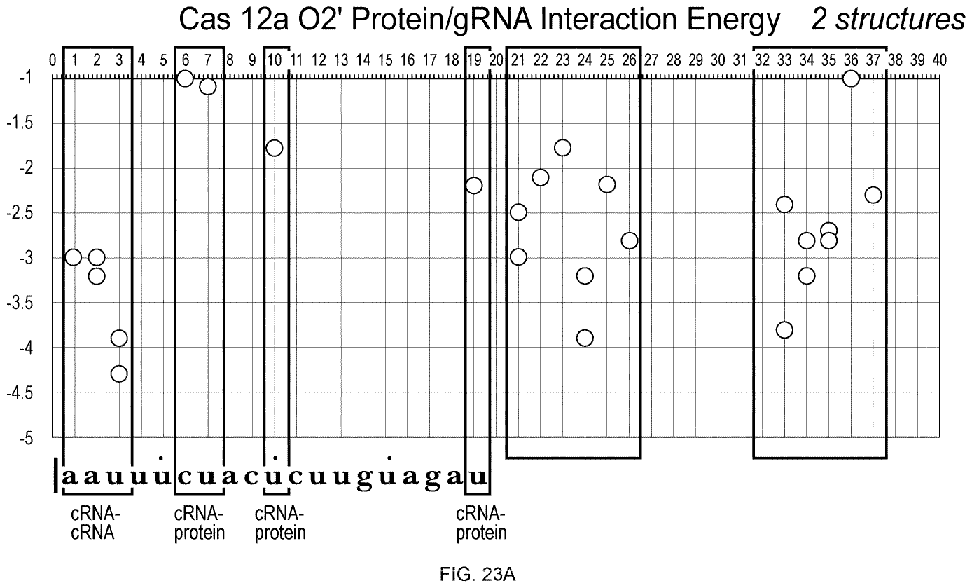

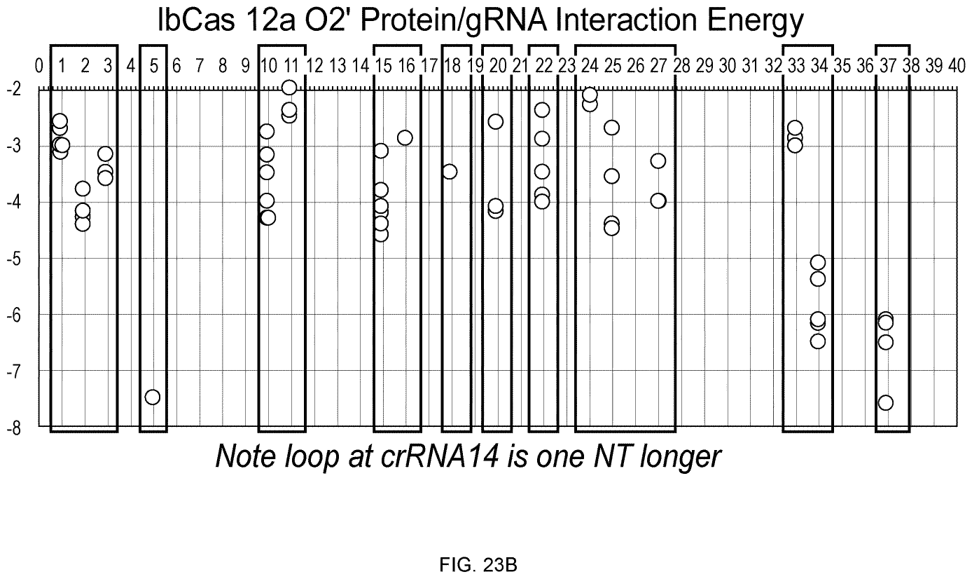

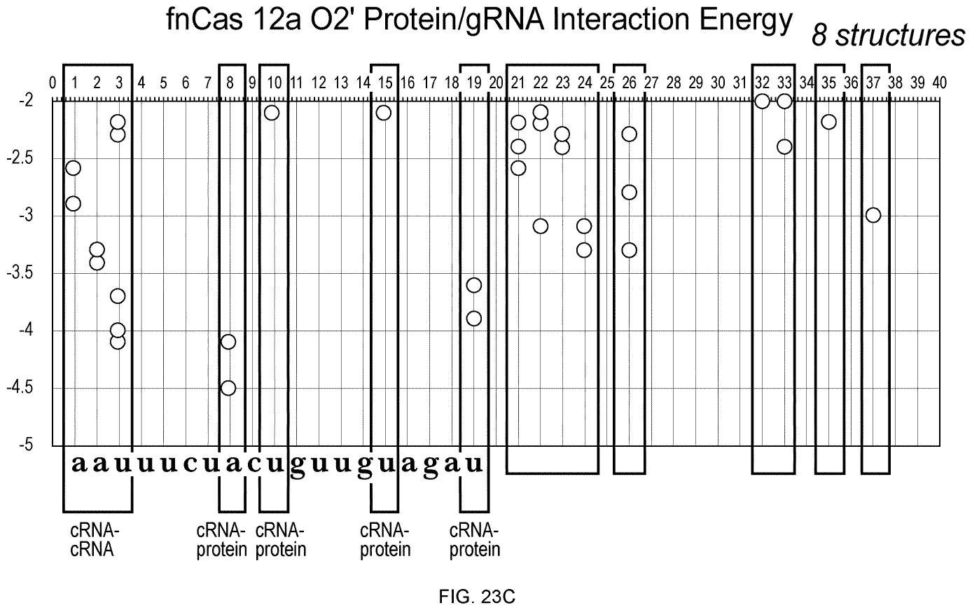

MOE analysis was performed with Cas12a guide bound protein structure. Based on MOE structural protocol, nucleotide positions were identified where the 2′-OH of gRNA nucleotide is making contact with Cas12 protein (FIGS. 22, 23, and 24). Cas12a O2′ protein/gRNA interaction energy can provide rationale for site-specific chemical modification of Cas12 gRNA.

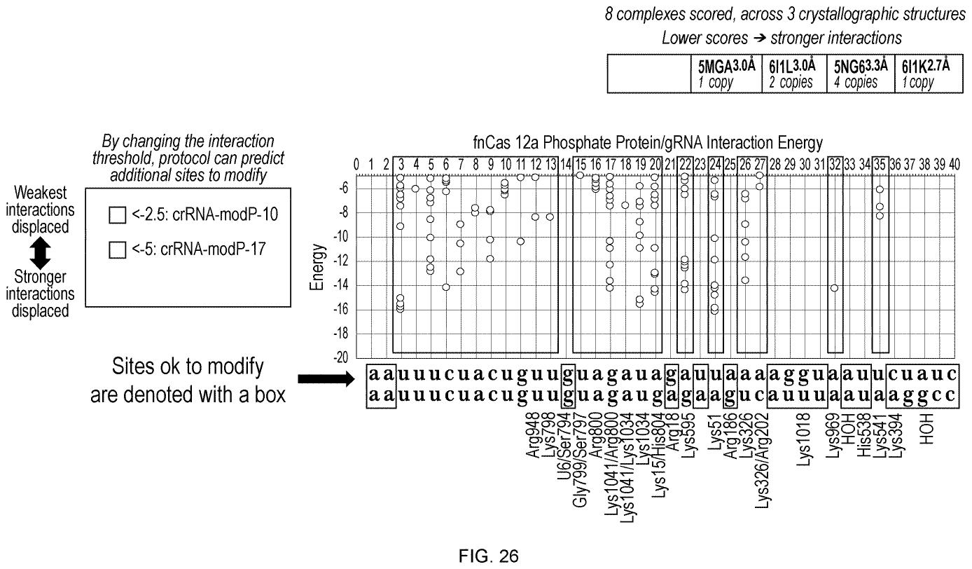

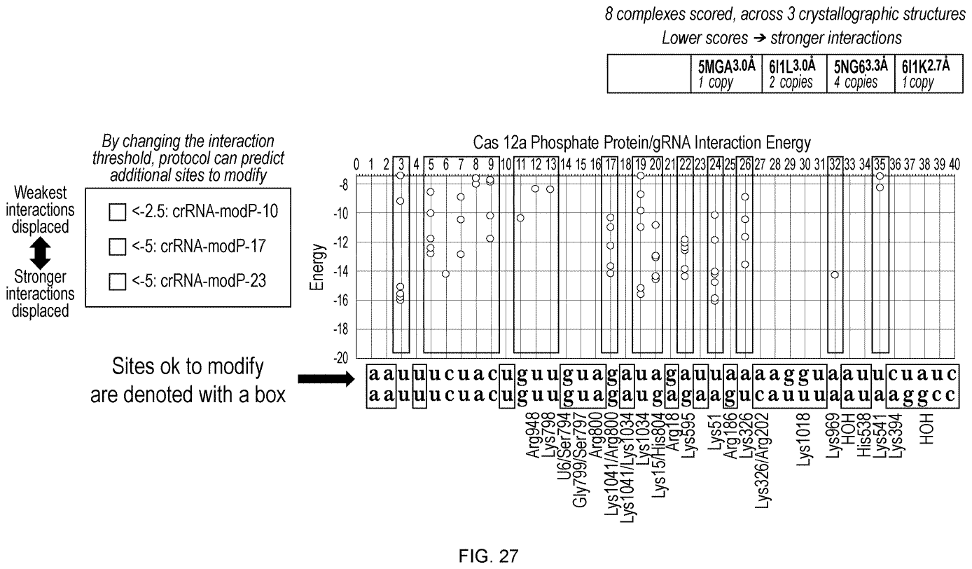

In FIG. 25, seven different chemical modification patterns were designed for gRNA of three different targets (target 1=PCSK9 gene; target 2=B2M gene; target 3=BCL11A binding site in the HBG1/2 promoters). Targeting this site with a CRISPR-Cas editor and guide RNA inactivates the binding site of the BCL11A transcriptional repressor, thereby increasing transcription of HBG1/2 genes, which results in increased production of HBG1/2 subunits, and consequently, increased production of HbF. Each of these three guides have the same direct repeat sequence (UGAAUUUCUACUGUUGUAGAU; SEQ ID NO: 192) but have different spacer length for unique DNA targets. In every guide, the first two and last two phosphates have been converted to phosphorothioate. According to MOE, position 3 may not be suitable for phosphorothioate modification (FIGS. 26 and 27) and hence were kept unmodified. This work involved working with 405-1 gRNA which has UG dinucleotide added before Cas12 guide sequence. For this work, these two nucleotides are assigned with −2 and −1 number. Following nucleotide sequence is identical with Cas12a gRNA direct repeat (AAUUUCUA . . . ) and they are assigned with 1, 2, 3 . . . as nucleotide identification number.

Every nucleotide was modified with 2′-OMe in the first sequence (SEQ ID NO: 193). This produces the maximum modification possible in this gRNA. The second sequence (SEQ ID NO: 194) has the direct repeat part completely modified. The third sequence has the spacer region completely modified. These two sequences inform the importance of modification of a specific portion of gRNA. Using MOE protocol, positions 1,2,3,10,14,19,23,24,25,32 and 35 were shown to have maximum interaction with Cas12 protein. They were not modified in the fourth sequence (SEQ ID NO: 196). In sequence five (SEQ ID NO: 197), all unmodified positions of sequence four were maintained. As such, additional unmodified positions (1,2,3,6,7,10,14,19,23,24,25,26,30,32,35,36,37) were added based on MOE protocol. This set includes positions which have moderate interaction with the protein. Sequence six (SEQ ID NO: 198) included less strong nucleotide interactions with the protein and included more unmodified positions (1,2,3,6,7,10,14,19,21,22,23,24,25,26,30,32,33,34,35,36,37). The first two nucleotides (position −2 and −1) are not part of Cas12 protein bound gRNA sequence, and hence their interaction with the protein cannot be measured using MOE protocol. To test whether they can accommodate chemical modification, a UU dinucleotide with 2′-OMe and PS was added (position −4 and −3) in sequence 7 (SEQ ID NO: 199) making position −2 and −1 unmodified. This modification pattern addresses the ability to accommodate chemical modification of the first two nucleotides in the 405-1 sequence.

This same strategy was applied to two other gRNAs targeting B2M and HBG with different spacer lengths. This generated 14 more gRNAs with similar chemical modification pattern (sequences 8-21 of FIG. 25; SEQ ID NOs: 200-213).

MOE citation: Molecular Operating Environment (MOE), 2022.02 Chemical Computing Group ULC, 910-1010 Sherbrooke St. W., Montreal, QC H3A 2R7, Canada, 2024.

### Example 17: Evaluation of Six (6) Modified Guides Under In Vivo Conditions Demonstrates Improved Editing Efficiency Compared to Unmodified Guides

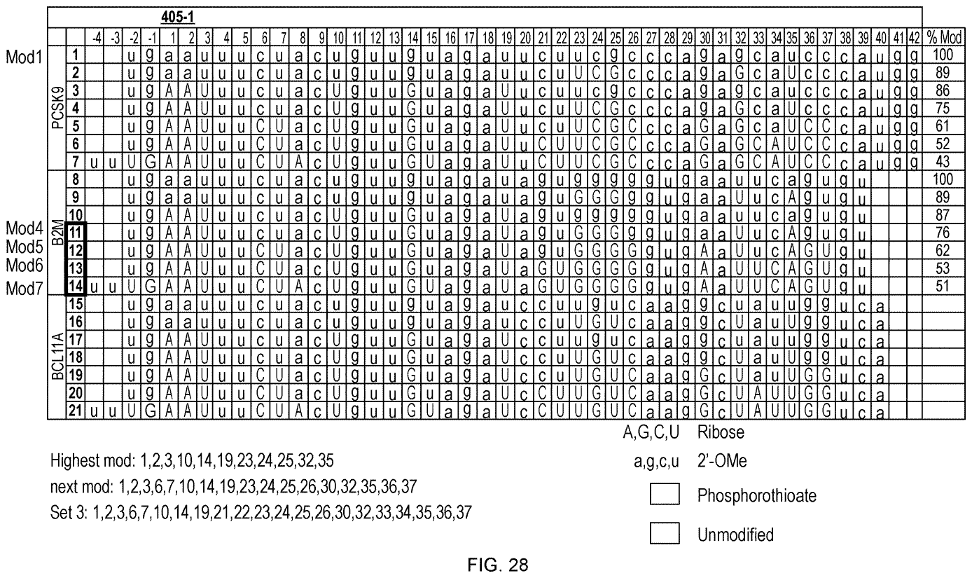

To evaluate whether the modified guides discussed in Example 16 resulted in improved editing efficiency, five (5) exemplary guides were tested. These selected guides are shown as “boxed” regions in FIG. 28.

For purposes of this example, the following nomenclature is used for the above modified guide RNAs of FIG. 28:

| | SEQ | |
|---|---|---|
| Designation | ID NO: | Sequence |
| Mod1 (Guide No. 1) | 214 | u*g*aauuucuacuguuguag |
| Target: PCSK9 | | auagugggggugaauucagu* |
| | | g*u |
| Mod4 (Guide No. 11) | 215 | u*g*AAUuucuacUguuGuag |
| Target: B2M | | aUaguGGGggugaaUucAgu* |
| | | g*u |
| Mod5 (Guide No. 12) | 216 | u*g*AAUuuCUacUguuGuag |
| Target: B2M | | aUaguGGGGgugAaUucAGU* |
| | | g*u |
| Mod6 (Guide No. 13) | 217 | u*g*AAUuuCUacUguuGuag |
| Target: B2M | | aUaGUGGGGgugAaUUCAGU* |
| | | g*u |
| Mod7 (Guide No. 14) | 218 | u*u*UUAAUuuCUAcUguuGU |
| Target: B2M | | agaUaGUGGGGgugAaUUCAG |
| | | U*g*u |

Legend: A, G, U, C: RNA nucleotide; a, g, u, c:
2′-O-Methyl-nucleotide; *: Phosphorothioate linkage

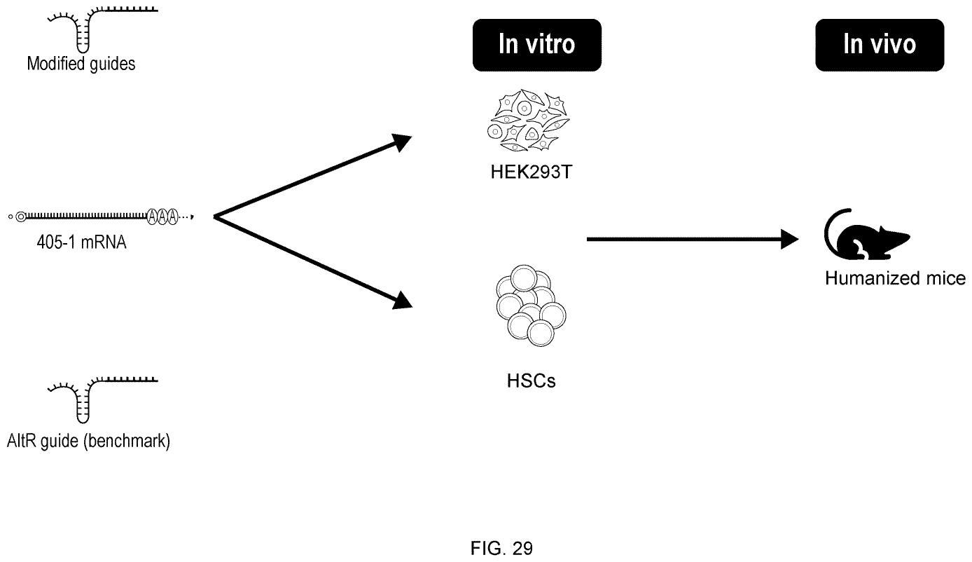

The modified guides Mod1, Mod4, Mod5, Mod6, and Mod7 were tested in accordance with the methodology depicted in FIG. 29.

As shown in FIG. 29, two different cells lines were transfected each with an mRNA encoding Cas12a ortholog variant 405-1 along with either a benchmark guide RNA (AltR guide) or one of the tested modified guides, Mod1, Mod4, Mod5, Mod6, or Mod7. In addition, the cells were transfected with a benchmark control combination of AsCas12a Ultra and the AltR guide. The transfections were as follows:

| Guide | Spacer | Transfected cell |
|---|---|---|
| RNA | target | type (in vitro) |
| Mod1 | PCSK9 | HEK293T and HSCs |
| Mod4 | B2M | HEK293T and HSCs |
| Mod5 | B2M | HEK293T and HSCs |
| Mod6 | B2M | HEK293T and HSCs |
| Mod7 | B2M | HEK293T and HSCs |

### Results

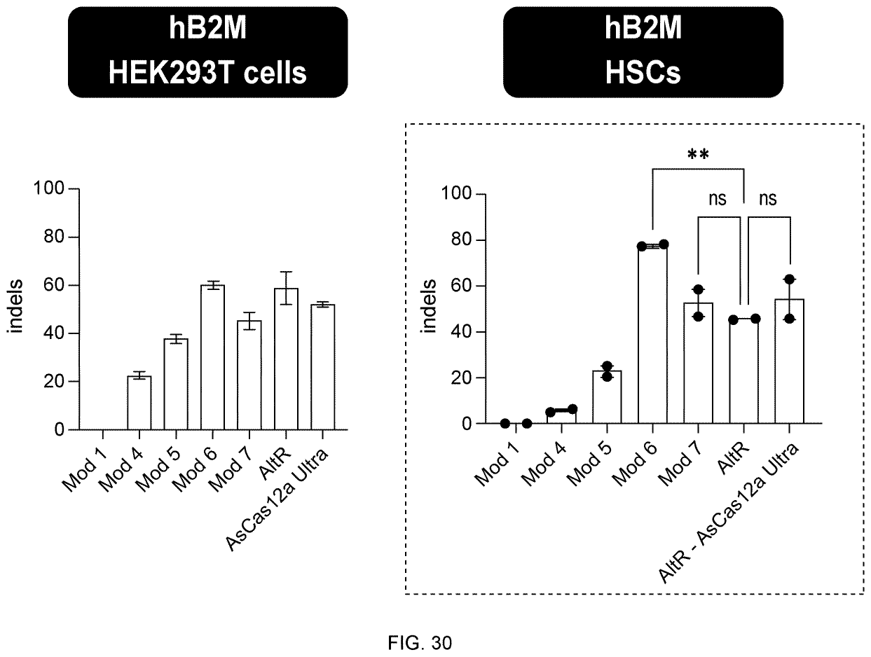

Results are shown in FIG. 30. The results demonstrate that:
Mod6 outperformed AltR control in primary HSCs with the B2M spacer guide
Cell type dependent differences were observed with guide performance
In primary HSCs, the guide modifications provided significant advantages over standard AltR guides.
Max editing achieved in HSCs with Cas12a variant 405-1 is 77%
Next steps including formulating modified guides in LNPs for in vivo studies

### Methods and Materials

### High Editing Rates in Primary HSCs at a Clinically Relevant Locus

Mobilized Human Peripheral Blood CD34+ Cells (AllCells, Donor ID #888824822) were thawed on Day 0. The following media was used:—X-VIVO 15 (Lonza; Cat #04-418Q), Recombinant Human SCF Protein, CF (R&D; Cat #11010-SC-050), Recombinant Human Thrombopoietin (NS0-expressed) Protein, CF (R&D; Cat #288-TPN/CF), Recombinant Human Flt-3 Ligand/FLT3L (HEK293) Protein, CF (R&D; Cat #308-FKHB-010), c-XVIVO Media (Complete X-VIVO)=X-VIVO+0.1 ug/mL SCF+0.1 ug/mL FLT3L+0.1 ug/mL TPO, 1×PBS (THERMOFISHER; Cat #10010049 or equivalent), 6 well plate (Corning; Cat #3516 or equivalent). RNA was electroporated into 100K cells using Lonza on Day 2 using 8 μg total RNA (at a ratio of 1:1) using pulse code DS130 according to the manufacturer's protocol. 48 hours later genomic DNA was extracted using prepGEM MicroGEM (Cat #PUN1000) according the manufacturer's protocol. NGS was performed on Illumina Nextseq and analysis by CRISPResso. mRNA encoding nuclease was manufactured by Vernal Biosciences and modified gRNA was manufactured by IDT. The gRNAs of SEQ ID NOs: 214-218 were used.

### Example 18: Targeting Guides to the Nucleus Via a PNS-NLS Localization Agent

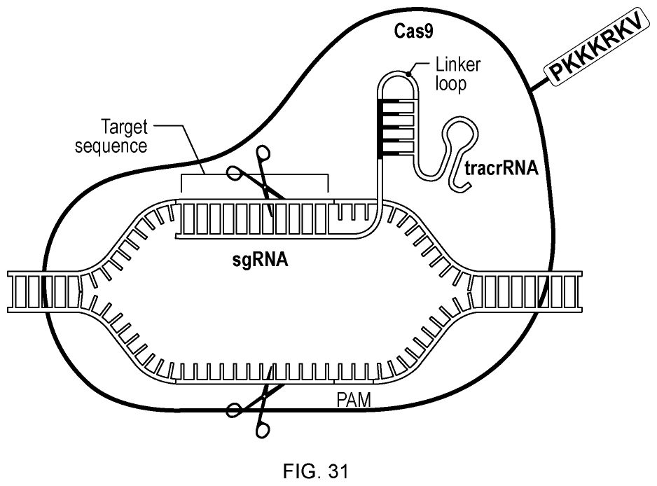

When delivering LNP-mediated CRISPR-based gene editing systems (e.g., which in various embodiments include mRNA and a gRNA), the mRNA needs to express a Cas nuclease in the cytoplasm, complex with its cognate guide RNA (to form the editing RNP—ribonucleoprotein—as depicted in FIG. 31), and then translocate into the nucleus to perform its editing function. In various embodiments, the Cas nuclease is engineered with one or more NLS sequences encoded thereon which direct the protein and/or complex to reach the nucleus. Guide RNA are unable to cross the nuclear membrane on their own and so the RNP complex must form in the cytoplasm prior to nuclear transport. Typically, to encourage RNP formation in the cytoplasm, a higher molar equivalence (10-150 fold) of guide RNA to Cas-encoding mRNA is encapsulated in the LNP. In addition, RNAs are more fragile than other genetic materials. This lack of stability leads to premature degradation while waiting for complexation with Cas protein. Selective site-specific chemical modification may increase stability and half of gRNA facilitating RNP formation, increasing gRNA potency. If guide RNA were modified such that it may translocate to the nucleus, editing efficiency may be increased as the RNP complex could form inside the nucleus.

As addressed in this Example, the problem of gRNA not being able to shuttle to the nucleus can be overcome by adding an NLS sequence directly to the guide RNA. By having an NLS sequence attached, the guide can cross the nuclear membrane and find and complex with a Cas protein which will have independently translocated into the nucleus. This is thought to significantly increase chances of RNP formation leading to improved gRNA potency. NLS sequence can be introduced to gRNA in multiple ways. Some non-limiting examples are stated below.
(A) By conjugating one or multiple peptide NLS sequences with a PNA probe having complementary sequence with portions of gRNA.
(B) By adding RNA-NLS sequence.
(C) By chemical conjugation of peptide NLS with gRNA.

Each of these approaches has unique advantages associated with them. PNA-NLS have a charge-free backbone and can form a stable duplex with short sequence overlap. Tracer sequence is conserved in all Cas9 gRNA sequences and the same PNA probe can be used in any guide for nuclear delivery of gRNA. This makes NLS-PNA probes a great choice for universal purposes. After binding with guide, the PNA will form a stable duplex on 3′-end of gRNA, this will enhance overall gRNA stability.

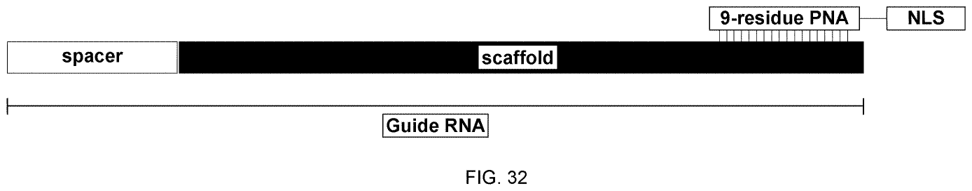

The concept of using a PNA-NLS probe is depicted in FIG. 32.

Various embodiments are embraced by these concepts and certain attributes may be optimized to increase performance.

For example, length of the PNA probe may be adjusted to regulate PNA-gRNA duplex stability. In certain embodiments, the PNA portion may range from 2-30 nucleotides, e.g. FIG. 32 which shows a 9-residue PNA linked to an NLS. The NLS sequence can be conjugated to either N-terminus or C-terminus of the PNA. In addition, the PNA probe may be conjugated to more than one NLS. It can also be conjugated internally with PNA probes. A few examples of the PNA-NLS probe are given below.

(SEQ ID NO: 219)-[linker]-PKKKRKV
OO-(SEQ ID NO: 219)
(SEQ ID NO: 219)-[linker]-PKKKRKV-[linker]-
Lys(Cy5)
OO-[linker]-AAAAGCACC
PKKKRKV-[linker]-AAAAGCACC
SEQ ID NO: 219 = AAAAGCACCGA

In these examples, the PNA peptide component may be coupled to one or more NLS. The coupling can optionally be by a linker.

In another embodiment, PNA probes may be modified to enhance solubility. PNA probes may in some embodiments be poorly insoluble, either on their own or when complexed with a target guide RNA. To address this potential issue, solubility enhancing groups like ethylene glycol (but not limited thereto) can be added to the PNA-NLS probe (shown by OO). The addition of a fluorophore (Cy5) can help to determine presence of gRNA in nucleus and cytoplasm. This will aid in identification of gRNA trafficking through nuclear membrane.

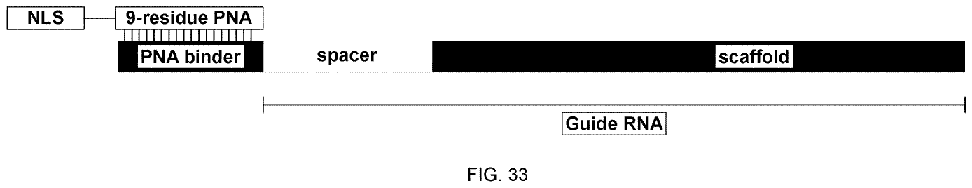

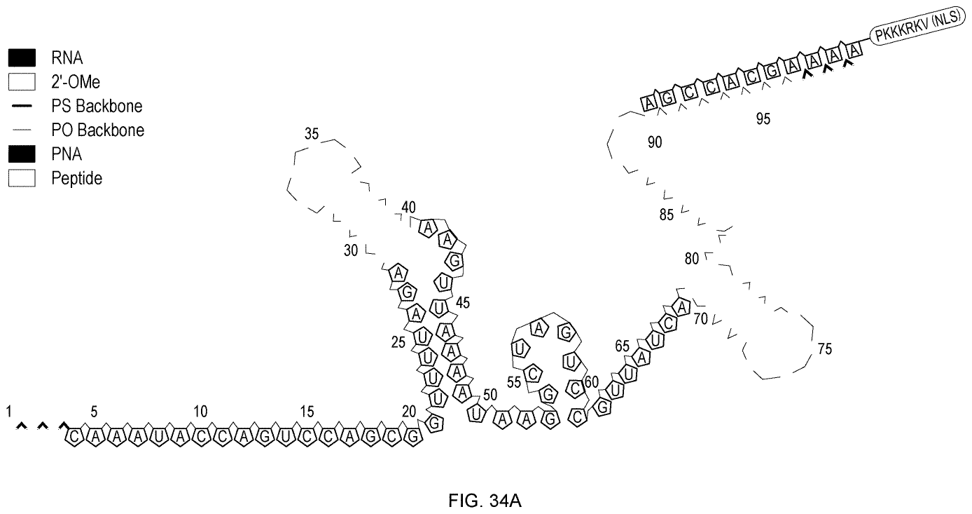

In various other embodiments, the PNA-NLS probes may also anneal to the 5′ end of a guide RNA at a specialized additional sequence that is added to the guide RNA at the 5′ or 3′ end names the “PNA binding sequence” or “PNA binder”. See FIG. 33.

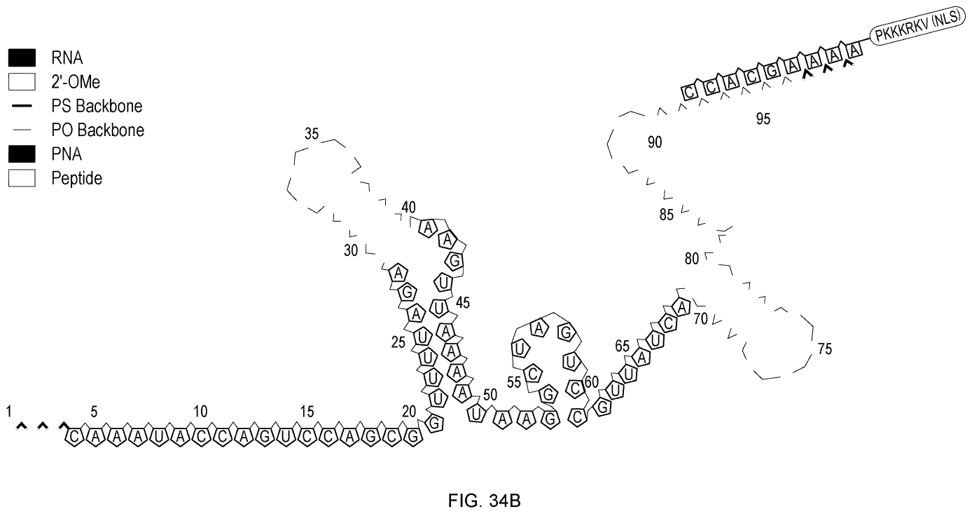

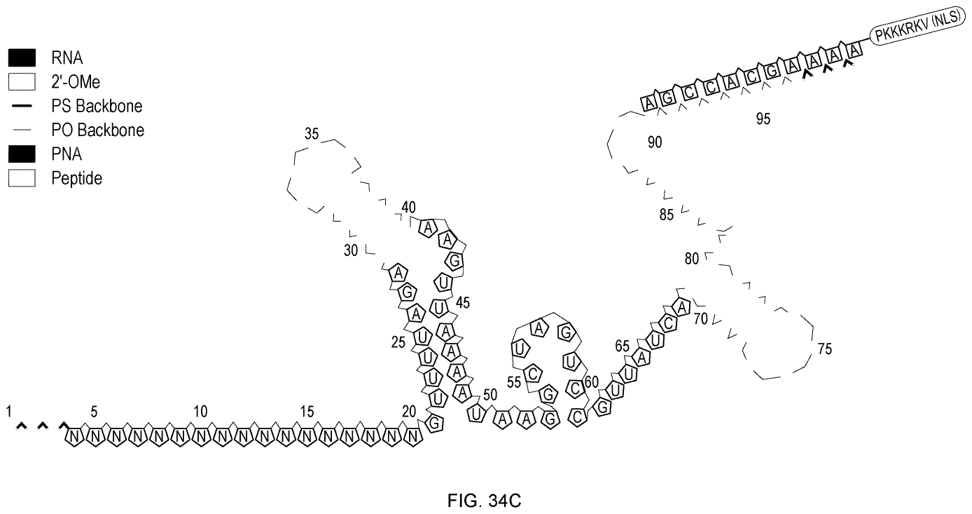

Exemplary additional embodiments of type V guides are shown in FIGS. 34A, 34B, and 34C.

### Example 19: Structurally Modified Guides Result in Improved Editing Efficiency (e.g., 3′ and 5′ RNA and/or DNA Extensions on Type V Guides Improves Editing Efficiency)

### Background

Genome-editing has come to the forefront of gene-therapy since the initial discovery of CRISPR/Cas9, a type II CRISPR nuclease. Despite the effectiveness of Cas9 for genome editing, several hurdles exist that make the CRISPR/Cas9 system difficult to work with for therapeutic development. Guide RNAs (gRNAs) for type II systems are typically 90-125 nts in length, which makes high-purity chemical RNA synthesis difficult. Furthermore, type II gRNAs must be heavily modified to ensure stability and efficacy when delivered in-vivo.¹ One solution to this problem is to use Cas proteins from a different family of nucleases which have different guide RNAs. Type V CRISPR/Cas systems are roughly the same size as Cas9 but leverage gRNAs that are only 30-50 nt in length, something that most RNA chemical synthesis platforms can accommodate.^{2,3} Furthermore, this shorter length means fewer residues need to be chemically modified, potentially stabilizing the gRNA at decreased cost. ReNAgade has previously described a proprietary type V nuclease, 405-1. Here we show that a variety of chemical and structural modifications to 405-1 gRNAs lead to more robust editing that simple chemical end modifications. Furthermore, these modifications are capable of rescuing “spacer dependent activity” often seen with type V gRNAs.²

### Results

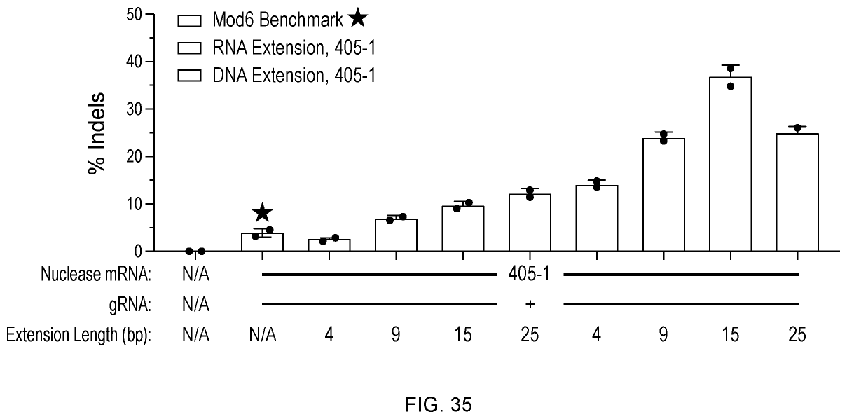

The extension of type V gRNAs at the 5′ end enhances editing at endogenous genomic sites as well as plasmid targets.³ Here chemically end-modified 405-1 nuclease gRNA were appended with RNA and DNA sequences of 4, 9, 15, and 25 bp, and introduced along with 405-1 mRNA into primary human CD34+ HSPCs. Two days after electroporation it was noted that appending RNA extensions increases editing 2-3 fold at the HBG locus above gRNAs containing our baseline 405-1 gRNA with chemical modifications in various positions (mod6/gRNA260) (FIG. 35).

This effect was length dependent as longer RNA sequences tended to increase editing levels. Interestingly, appending the same sequences as DNA resulted in editing levels 4-10 fold above the baseline mod6 gRNA. The baseline mod6 gRNA has the following sequence: gRNA0260: u*g*AAUuuCUacUguuGuagaUcCUUGUCaagGUAUUGGU*c*a (SEQ ID NO: 680) (aka “mod6” guide), wherein A, G, U, C=unmodified RNA nucleotide; a, g, u, c=2′-O-Methyl-nucleotide; *=Phosphorothioate linkage.

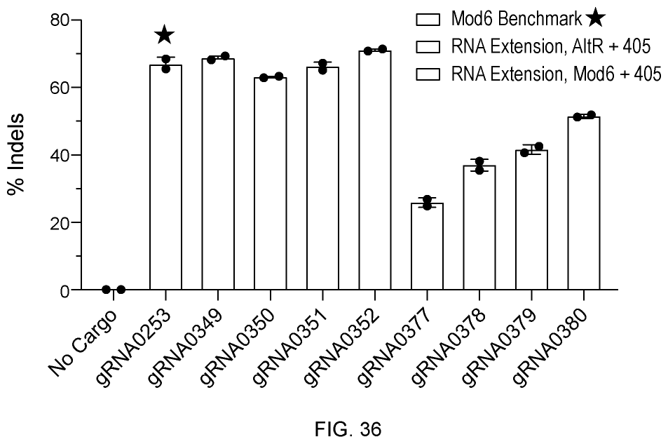

These results were also somewhat length dependent but optimal editing occurred with an extension of 15 bp, rather than an extension of 25 bp. These effects were not observed with a type V gRNA targeting B2M in HSPCs, potentially because the gRNA caused robust editing at baseline (FIG. 36). These results suggest that the incorporation of 5′ extensions into type V gRNAs can increase editing levels and that DNA extensions tend to increase editing more than RNA extensions using spacers with initial poor activity. It will be interesting to determine if these results are due to increases in gRNA stability or improved affinity for the 405-1 nuclease.

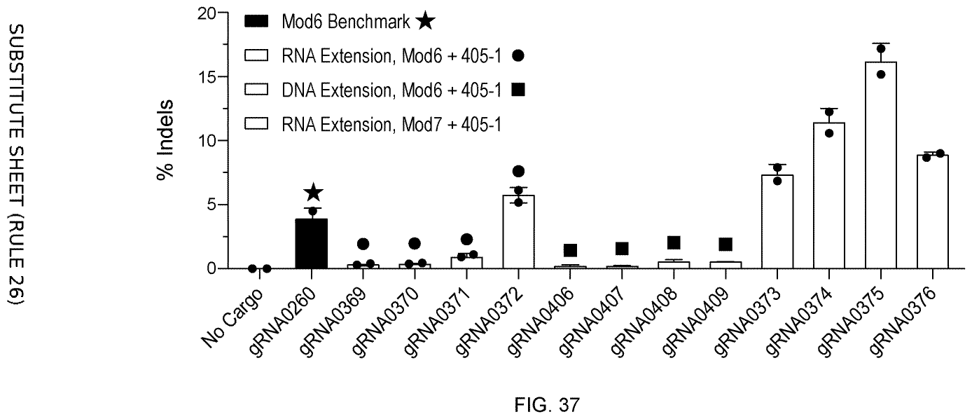

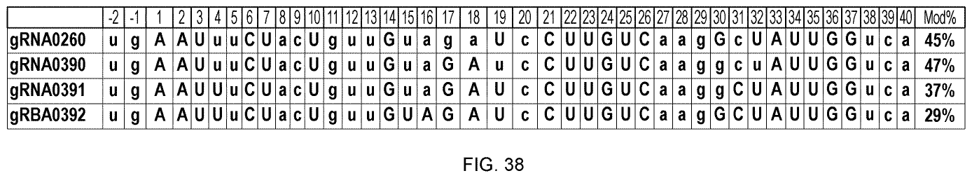

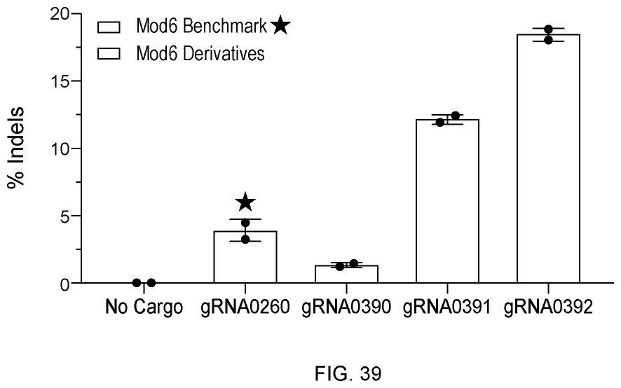

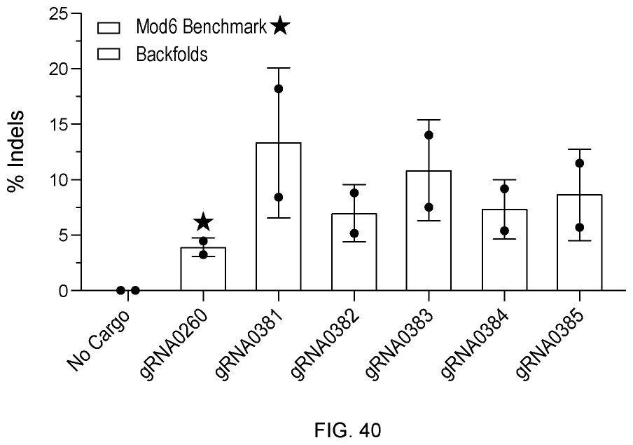

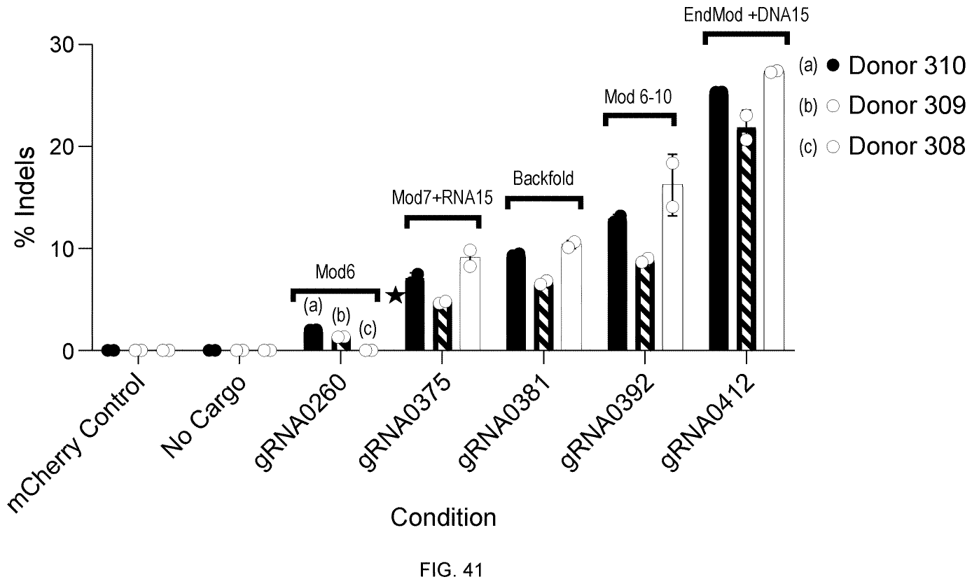

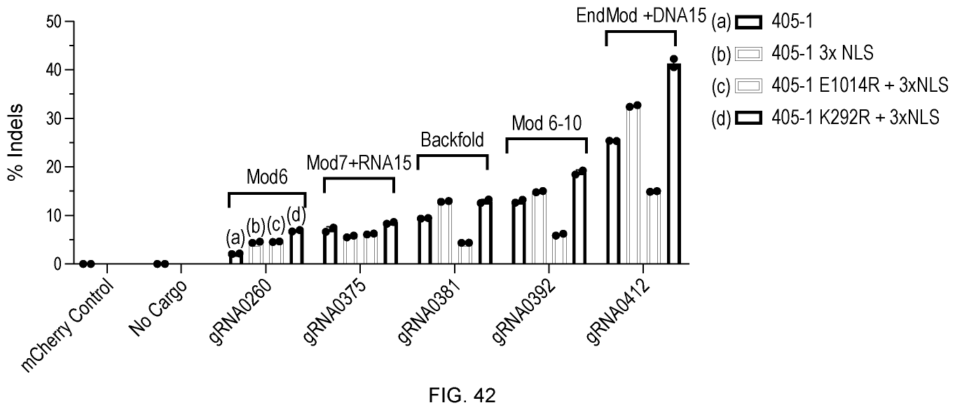

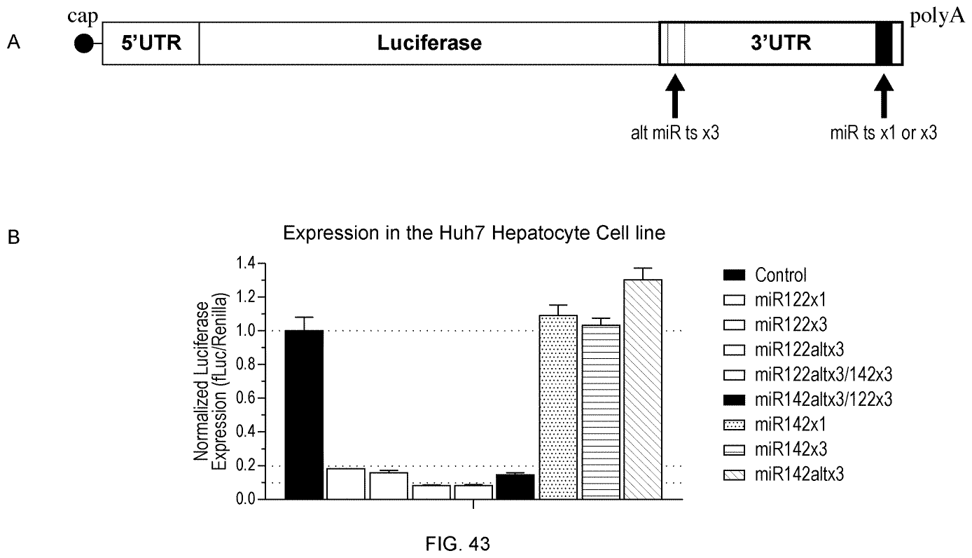

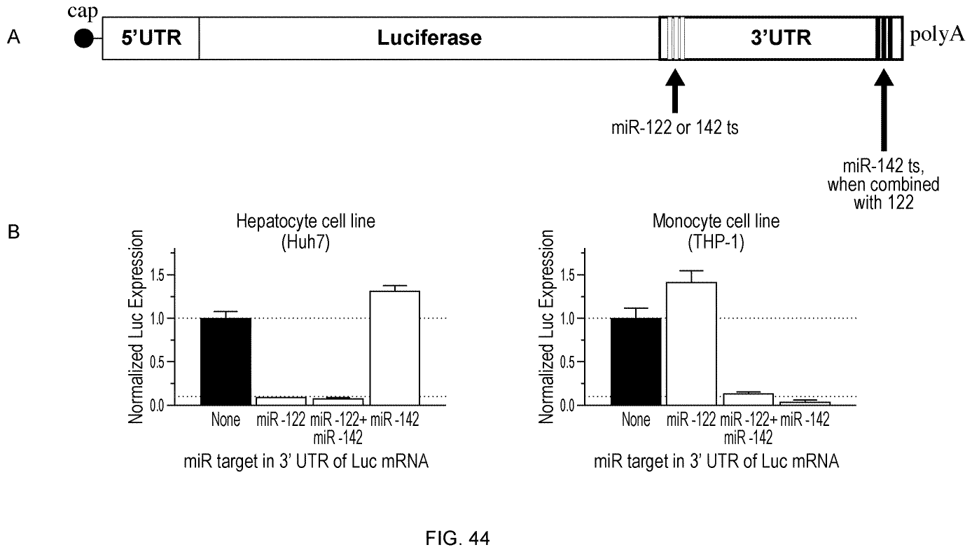

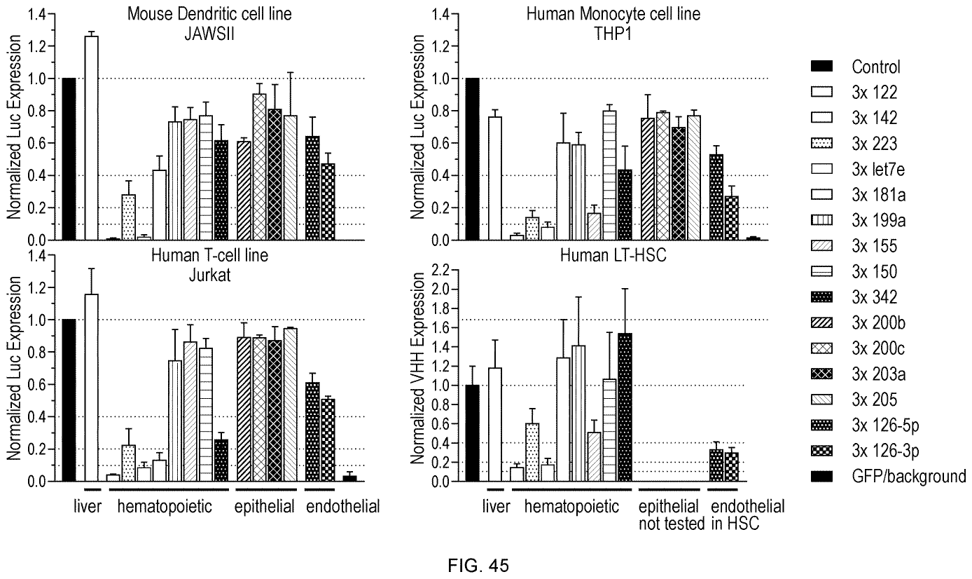

Next, the mod6 schema were combined with RNA extensions to determine if further improvements in editing could be observed in HSPCs. It was observed that most RNA extensions, except for a 25 bp extension (gRNA0372), decreased editing below the levels observed with mod6 alone (gRNA0260) at both HBG and B2M loci (FIGS. 36 and 37). The 25 bp extension boosted editing frequencies ~1.5-2 fold. This effect could be due to an extended stretch of polyuracil in the extension sequence that could be acting as a linker, separating the gRNA direct repeat from the flanking extension sequence.

Conversely, it was noted that modification scheme 7 (mod7), which was less effective in HSPCs than mod6, could be modified with RNA extensions (gRNA0373-0376) and improved editing 3-fold at the HBG locus, with an optimal extension length of 15 bp (gRNA0375). Adding DNA extensions on mod7 gRNAs may further improve editing effects. Mod7 has the sequence and modification schema, as follows: u*u*UGAAUuuCUAcUguuGUagaUcCUUGUCaagGUAUUGGu*c*a, A, G, U, C=unmodified RNA nucleotide; a, g, u, c=2′-O-Methyl-nucleotide; *=Phosphorothioate linkage is SEQ ID NO: 213.

As gRNA0260 originally improved editing in HSPCs, it was tested whether derivatives of gRNA0260 could improve activity. Three derivatives were chosen based on the overall level of modification in the gRNA, gRNA0390 (mod6-6; SEQ ID NO: 681), gRNA0391 (mod6-8; SEQ ID NO: 682), and gRNA0392 (mod6-10; SEQ ID NO: 683), and electroporated in human HSPCs (FIG. 38). It was noted that editing levels increased as gRNAs were less modified and approached a 10-fold improvement, gRNA0392, over the initial design using gRNA0260 (FIG. 39). This suggests that some modifications are impacting the gRNA folding and/or gRNA nuclease interactions and impairing editing in some ways.

Lastly, fold-back gRNAs can promote proper folding of type V gRNAs which could rescue spacers with poor editing activity.² Five different fold-back gRNAs were designed with 3′ extensions pairing with the 3′ end of the spacer sequence and introduced them into human HSPCs. It was noted that the editing frequencies with fold-back RNAs improved the editing more than mod6 (gRNA260) but had significant variability (FIG. 40).

To confirm that these results were not HSPC donor specific, these experiments were repeated in multiple HSPC donors and it was found that DNA extensions, mod6-10, and foldback increased editing across all three donors (FIG. 41). Notably, the foldback designs generated editing results there were more consistent than the prior experiment and better than the mod6 benchmark. These RNA modifications were also combined with 405-1 nuclease that contained an optimized NLS configuration and either E1014R or K292R point mutants. It was noted that editing was generally increased in the presence of optimized NLSs and increased further when combined with K292R (FIG. 42). Addition of E1014R to 405-1 with optimized NLSs did not improve editing and, in most cases, decreased editing by two-fold.

### Discussion

Here, multiple structural modifications have been incorporated into type V gRNAs to improve editing rates in primary human HSPCs. The addition of 5′ DNA extensions, 5′ RNA extensions, reduced chemical modification (mod6-10), and hairpins around the spacer region (backfolds) all led to two-fold or greater improvements over the benchmark mod6 gRNA in multiple HSPC donors. While structural modifications all lead to increases in editing with chemical end modifications, some were not compatible with the mod6 schema. A different modification scheme (mod7) could support RNA extensions that improved editing. DNA extensions, RNA extensions, foldbacks, and mod6-10 gRNAs were also augmented with NLS and point mutant improvements in the 405-1 nuclease showing that these elements can be combined to augment editing. Combining these gRNA modifications may yield more robust editing than seen with a single modification alone.

### Materials and Methods

Mobilized CD34+ HSPCs were ordered from AllCells. All guide RNAs were ordered from IDT. Guide RNA schemas tested included the following:
IDT's proprietary AltR modification pattern
2′O-Me modifications and phosphothiorate linkages at the first and last two nucleotides
2′O-Me modifications and phosphothiorate linkages at the first and last two nucleotides, with proprietary 2′O-Me modifications interspersed along the guide length
Chimeric guides including DNA extensions with phosphothiorate linkages at the first and last two nucleotides and 2′O-Me modifications on the last two nucleotides
405-1 was manufactured through Vernal Biosciences. N1014R+3×NLS and K291R+3×NLS nuclease variants were produced by the inventors.

### Cell Culture:

Primary human CD34+ HSPCs were cultured in X-Vivo 15 medium (Lonza Catalog #: 04-418Q) supplemented with 100 ng/mL SCF (RnD Systems Inc. 11010-SC-050), 100 ng/uL FLT-3L (RnD Systems Inc. 308-FKHB-010), and 100 ng/uL recombinant human thrombopoetin (RnD Systems Inc. 288-TPN/CF). HSPCs were thawed and cultured for two days before electroporation.

### Electroporations:

Mobilized primary human CD34+ HSPCs were electroporated using a Lonza 4D 96-well electroporator and a Lonza P3 Primary cell kit (Catalog #: V4SP-3096). Briefly, 100,000 HSPCs were resuspended in Lonza P3 buffer and combined with 4000 ng of guide and 4000 ng of nuclease mRNA to a total volume of 20 μL per replicate, per condition. The samples were electroporated using the DS-130 program, 100 μL of pre-warmed cell culture medium was added to the cells immediately after electroporation, and the entire mixture transferred to a pre-warmed 96-well plate containing 100 μL of cell culture medium. Samples were allowed to grow at 37 C 5% CO₂ for two days before they were centrifuged, and stored as frozen pellets for genomic DNA (gDNA) isolation using the Mag-Bind® Blood & Tissue DNA HDQ 96 Kit (Omega Bio-tek Cat #: M6399) according to the manufacturer's instructions.

### Next Generation Sequencing and Analysis

Using extracted gDNA as template, the respective samples containing either HBG or B2M sites of interest were amplified by PCR and prepared for Illumina sequencing. The resulting data was analyzed using CRISPREsso2.

### References

1. Yin, H., Song, C Q., Suresh, S. et al. Structure-guided chemical modification of guide RNA enables potent non-viral in vivo genome editing. Nat Biotechnol 35, 1179-1187 (2017). https://doi.org/10.1038/nbt.4005
2. Creutzburg S C A, Wu W Y, Mohanraju P, Swartjes T, Alkan F, Gorodkin J, Staals R H J, van der Oost J. Good guide, bad guide: spacer sequence-dependent cleavage efficiency of Cas12a. Nucleic Acids Res. 2020 Apr. 6; 48(6):3228-3243. doi: 10.1093/nar/gkz1240. PMID: 31989168; PMCID: PMC7102956.
3. Park H M. et. al Extension of the crRNA enhances Cpf1 gene editing in vitro and in vivo. Nature Comm. (2018)

### Example 20: Cell-Specific Regulation of mRNA Expression May be Controlled by miR Sites

### Introduction

Lipid nanoparticles (LNPs) can effectively protect mRNA from degradation and facilitate its efficient delivery in vivo, as demonstrated in the development of COVID-19 vaccines. Following intravenous (IV) administration, the majority of mRNA-LNP complexes tend to accumulate in the liver, leading to the expression of cargo proteins by hepatocytes or sinusoidal endothelial cells. Additionally, myeloid cells in the liver or spleen often engulf these complexes and express cargo proteins as part of their physiological function. While efforts are ongoing to improve the precision of mRNA-loaded LNP delivery to specific tissues beyond the liver, another approach involves engineering the mRNA cargo sequence to mitigate expression in off-target tissues. Such a strategy could be implemented to ensure more specific delivery to a desired target cell or tissue, such as HSCs in bone marrow.

MicroRNAs (miRNAs) are short, non-coding single-stranded RNAs, typically 18-24 nucleotides in length, that regulate gene expression by binding to target RNA sequences. A perfect match between a miRNA and its target sequence controls mRNA stability, while an imperfect match leads to inhibition of translation for protein-coding mRNAs. By selecting miRNAs that are highly expressed in off-target tissues but not in the desired tissues, and incorporating target sequences for these miRNAs into the untranslated regions (UTRs) of the cargo mRNA, the tissue-specificity of therapeutic protein expression can be modulated.

Suppression of Cargo mRNA Expression by Endogenous miRNA and miRNA Target Site in the UTR of Cargo mRNA

This example establishes a platform for miR-mediated suppression of mRNA expression in a tissue culture model (FIG. 43). In FIG. 43(A), a schematic representation of luciferase mRNA with microRNA (miRNA) target sites (“ts”) inserted in the 3′UTR is shown. Luciferase mRNA consists of 5′ cap, 5′UTR, luciferase coding sequence, 3′UTR followed by a poly A tail. A single copy (×1) or three copies (×3) of perfectly matched miRNA target sequences were inserted at the 3′ end of the 3′ UTR. An alternative (alt) insertion site is located at the 5′ end of the 3′UTR, near the luciferase coding sequence. When combining two miRNA target sites in the same UTR, the first miRNA target site is inserted at the ‘alt’ location, and the second miRNA target site is inserted at the 3′ end of UTR. In FIG. 43(B), the incorporation of miR-122 ts into the 3′ UTR of luciferase mRNA results in the suppression of encoded protein in the Huh7 hepatocyte cell line, where miR-122 is expressed. A single copy of the target site at the end of 3′ UTR near poly A tail is sufficient to suppress luciferase expression by 5-fold compared to the control. Increasing the number of copies to three does not further enhance suppression. However, when the target site is inserted at the 5′ end of UTR, near the coding sequence, suppression is enhanced to 10-fold. In contrast, insertion of the target site of miR-142, which is not expressed in the hepatocyte cell line, has no impact on luciferase expression by itself and does not influence miR-122 mediated suppression when both target sites are inserted together on the same UTR.

This data demonstrates that endogenously expressed miRNAs, along with the incorporation of miRNA target sites in cargo mRNA, can effectively suppress cargo expression in the tissue where the respective miRNA is expressed.

Tissue-Specific Expression of Cargo mRNA is Achieved Through the Incorporation of Specific miR Target Sites in the Cargo mRNA

To investigate whether tissue-specific expression of cargo mRNA could be achieved by miRNAs, cell lines were utilized that specifically express certain miRNAs. Huh-7 hepatocyte cell line was selected, which expresses miR-122, and the THP-1 monocyte cell line, which expresses miR-142, in a cell type-specific manner. In the experimental setup, three copies of miRNA target site were inserted at the 5′ end of 3′ UTR, near the luciferase coding sequence (FIG. 44A). When combining two miRNA target sites in the same UTR, the first miRNA target site was inserted near the luciferase coding sequence and the second miRNA target site was inserted at the 3′ end of UTR. The insertion of miR-122 ts into the 3′ UTR of luciferase mRNA led to the suppression of protein expression in the hepatocyte cell line, where miR-122 is expressed (FIG. 44B, left), but not in the monocyte cell line, where miR-122 is not expressed (right). Conversely, miR-142 ts suppressed luciferase expression only in the monocyte cell line, where miR-142 is expressed (FIG. 44B, right). Inserting both miR-122 and miR-142 target sites into the same UTR inhibited luciferase expression in both the hepatocyte and monocyte cell line. This suggests the potential use of multiple miRNA target sites to suppress expression in various off-target tissues.

Testing miRNA-Mediated Suppression Across Various Immune Cells and Hematopoietic Stem Cells

Table 22 lists miRNAs expressed in liver, myeloid cells and other immune cells, hematopoietic stem cells, epithelial, and endothelial cells. All fifteen variants of firefly luciferase or VHH mRNA were synthesized, each containing three copies of a unique miRNA target site. These target sites were inserted at the ‘alt’ location of the 3′UTR, near the coding sequences. In FIG. 45, the miRNA target-site mediated suppression was assessed in various immune cell lines and human long-term hematopoietic stem cells (LT-HSCs). The LT-HSCs were identified as CD34+CD38-CD90+CD45RA−Lineage− by surface expression of those markers among bone marrow-derived CD34+ cells. While the insertion of liver specific miR-122 or epithelial-specific miR-200b, 200c, 203a and 205 target sites in the 3′ UTR of cargo does not affect its expression in immune cells, hematopoietic miRNAs suppress cargo expression in one or more cell types. miR-142 and let7e effectively suppress cargo expression in all cell lines. miR-223 exhibits mild activity in all cell lines, with the least effect observed in long-term hematopoietic cells. miR-155 demonstrates suppression in the monocyte cell line and in long-term hematopoietic cells, albeit weaker in the latter. miR-342 suppresses expression in both monocyte and T-cell lines. Both mir-126 5p and 3p, which are abundantly expressed in endothelial cells, show a substantial suppressive effect in long-term hematopoietic stem cells.

This data suggests that incorporating both miR-122 and miR-223 target sites into the cargo sequence could be beneficial for hematopoietic stem cell (HSC) targeting therapy, as it helps suppress expression in off-target tissues such as the liver and myeloid cells, mitigating potential liver toxicity or innate immune activation.

### Methods and Materials

### Mammalian Cell Culture

Huh-7 cells (ATCC CRF-3216) were cultured in Dulbecco's modified Eagle's medium (DMEM) plus GlutaMAX (Thermo Fisher Scientific), supplemented with 10% (v/v) fetal bovine serum (Gibco). JAWSII murine DCs (ATCC: CRL-11904) were cultured in α-MEM, supplemented with 20% (v/v) fetal bovine serum (Gibco), 2 mM GlutaMAX (Gibco), and 5 ng/ml murine GM-CSF (Sigma). THP1 dual cells (InvivoGen) were cultured in RPMI 1640, supplemented with 10% (v/v) fetal bovine serum (Gibco), 2 mM Glutamine (Gibco), 10 μg/mL of Blasticidin (InvivoGen), and 100 μg/mL of Zeocin (InvivoGen). Jurkat cells (ATCC #TIN-152) were cultured in RPMI 1640, supplemented with 10% (v/v) fetal bovine serum (Gibco), 2 mM GlutaMAX (Gibco) and HEPES (Gibco). Human Bone Marrow derived CD34+ cells (StemCell Technologies) were cultured in X-VIVO 15 media (Lonza) supplemented with 100 ng/ml of SCF, FLG-3L, and TPO (Peprotech). Cells were maintained at 37° C. with 5% CO₂.

### In Vitro Transcription

Plasmid templates were synthesized by Twist Bioscience. T7 promoter was inserted upstream of RT or ncRNA sequences. Plasmid was linearized by Sap1 enzyme (NEB, R0569L) for 30 minutes at 37° C. and the complete linearization was checked by agarose gel electrophoresis. Linearized template was purified by GeneJET PCR purification kit (K0701, Thermo Fisher Scientific) and quantified by NANODROP. mRNA was synthesized for 2 hours at 37° C. using HiScribe T7 mRNA kit with CleanCap Reagent AG (NEB, E2080S) following manufacturer's protocol. After DNAse1 treatment, RNA was purified using Qiagen RNeasy midi kit (Qiagen, 75144). RNA concentration was measured by NANODROP (Thermo Fisher) and purity and size were evaluated by RNA ScreenTape analysis on Tape station (Agilent). RT mRNA was further poly(A) tailed by E. coli Poly(A) polymerase (NEB, M0276) and purified by Qiagen RNeasy midi kit. Tail length was estimated by RNA ScreenTape analysis by comparing before and after poly(A) adenylation.

RNA Transfection by messengerMax, TransIT, and jetMESSENGER Reagents

15,000~30,000 cells of Huh-7 hepatocytes, JAWSII murine DCs, Jurkat T-cells, and THP1 dual cells were seeded in 96 well plates. 16-24 hours post-seeding, cells were transfected with appropriate amount of RNA mixture complexed with Lipofectamine MessengerMax (Thermo Fisher Scientific) for Huh-7, or TransIT (Mirus Bio) for JAWSII or jetMESSENGER (polypus) for THP-1. Cells were cultured for 24 hours before measuring protein expression.

### RNA Transfection by Neon Electroporation System

Human CD34+ cells were harvested and resuspended in T buffer. 50,000 cells with 1 μl of RNA mixture were electroporated by 10 μl Neon tip at 1600 voltage, 10 pulse width, 3 pulses. Electroporated cells were gently transferred to 96 well plate (VWR) containing prewarmed culture media and cultured for 24 hours before analyzing by flow cytometry.

### Measurement of Luciferase Expression

Amount of luciferase protein was measured by ONE-Glo™ EX luciferase assay system (Promega E8120). After equilibrating the reagent and cells to room temperature, equal volume of reagent and cells were mixed and incubated for 3 minutes on an orbital shaker at 300-600 rpm. The luminescence was read on Varioskan LUX (Thermo Fisher Scientific) at 200 msec integration time. Luminescence from Firefly luciferase was normalized by that from Renilla luciferase.

### Flow Cytometry Analysis of VHH Expression in Human Long-Term Hematopoietic Stem Cells

Culture media was removed by centrifuging the culture plate. Cells were washed with PBS and incubated with Live Dead Aqua (INVITROGEN) for 15 minutes at room temperature. Cells were washed with cell staining buffer (BIOLEGEND, 420201) and incubated with Human Fc Block (BIOLEGEND, 422302) for 10 minutes on ice. Antibody cocktail comprising anti-CD38 (FITC, StemCell Technologies), anti CD45 (APC-Fire750, BIOLEGEND), anti-CD34 (BV421, BIOLEGEND), anti-CD45RA (BV605, BD biosciences), anti-CD90 (PE-Cy7, BIOLEGEND), anti-CD3 (AF700, BD biosciences), anti-CD8 (AF700, BIOLEGEND), anti-CD11b (AF700, BIOLEGEND), anti-CD11c (AF700, BIOLEGEND), anti-CD14 (AF700, BIOLEGEND), anti-CD19 (AF700, BIOLEGEND), anti-CD56 (AF700, BIOLEGEND), and anti-CD235a (AF700, BIOLEGEND) was added to the cells and incubated for 30 minutes on ice in the dark. After incubation, cells were washed twice with cell staining buffer and acquired on Attune NxT flow cytometer (Thermo Fisher Scientific).

TABLE 22
Select miRNA Sequences

| | | Full-complement Target | Targeted |
|---|---|---|---|
| miRNA | miRNA Mature Sequence | Sequence | Tissue |
| miR-122-5p | UGGAGUGUGACAAUGGU | CAAACACCATTGTC | liver |
| | GUUUG (SEQ ID NO: 721) | ACACTCCA (SEQ ID NO: 736) | |
| miR-142-3p | UGUAGUGUUUCCUACUU | TCCATAAAGTAGGAA | hematopoietic |
| | UAUGGA (SEQ ID NO: 722) | ACACTACA (SEQ ID NO: 737) | |
| miR-223-3p | UGUCAGUUUGUCAAAUA | TGGGGTATTTGACAA | |
| | CCCCA (SEQ ID NO: 723) | ACTGACA (SEQ ID NO: 738) | |
| miR-155-5p | UUAAUGCUAAUCGUGAU | AACCCCTATCACGAT | |
| | AGGGGUU (SEQ ID NO: 724) | TAGCATTAA (SEQ ID NO: 739) | |
| miR-150-5p | UCUCCCAACCCUUGUAC | CACTGGTACAAGGGT | |
| | CAGUG (SEQ ID NO: 725) | TGGGAGA (SEQ ID NO: 740) | |
| miR-199a-3p | ACAGUAGUCUGCACAU | TAACCAATGTGCAGAC | |
| | UGGUUA (SEQ ID NO: 726) | TACTGT (SEQ ID NO: 741) | |
| miR-342-3p | UCUCACACAGAAAUCGC | ACGGGTGCGATTTCTG | |
| | ACCCGU (SEQ ID NO: 727) | TGTGAGA (SEQ ID NO: 742) | |
| miR-181a-5p | AACAUUCAACGCUGUCG | ACTCACCGACAGCGTT | |
| | GUGAGU (SEQ ID NO: 728) | GAATGTT (SEQ ID NO: 743) | |
| let-7e-5p | UGAGGUAGGAGGUUGUA | AACTATACAACCTCCT | |
| | UAGUU (SEQ ID NO: 729) | ACCTCA (SEQ ID NO: 744) | |
| miR-200b-3p | UAAUACUGCCUGGUAA | TCATCATTACCAGGC | epithelial |
| | UGAUGA (SEQ ID NO: 730) | AGTATTA (SEQ ID NO: 745) | |
| miR-200c-3p | UAAUACUGCCGGGUAA | TCCATCATTACCCGG | |
| | UGAUGGA (SEQ ID NO: 731) | CAGTATTA (SEQ ID NO: 746) | |
| miR-203a-3p | GUGAAAUGUUUAGGAC | CTAGTGGTCCTAAAC | |
| | CACUAG (SEQ ID NO: 732) | ATTTCAC (SEQ ID NO: 747) | |
| miR-205-5p | UCCUUCAUUCCACCG | CAGACTCCGGTGGAA | |
| | GAGUCUG (SEQ ID NO: 733) | TGAAGGA (SEQ ID NO: 748) | |
| miR-126-5p | CAUUAUUACUUUUG | CGCGTACCAAAAGT | endothelial |
| | GUACGCG (SEQ ID NO: 734) | AATAATG (SEQ ID NO: 749) | |
| miR-126-3p | UCGUACCGUGAGUA | CGCATTATTACTCAC | |
| | AUAAUGCG (SEQ ID NO: 735) | GGTACGA (SEQ ID NO: 750) | |

APPENDIX
SEQUENCE LISTING

| SEQ ID | | Notes |
|---|---|---|
| NO | SEQUENCE | (if relevant) |
| 1 | GGAAAUCGCAAAAUUUGCUCUUCGCGUUAGAUUUCUUUUAGUUUUCUCGCAACUAGCAAGCUUUUUGUUCUCGCCGCCGCC | |
| 2 | GGAAAUCGCAAAAUUUUCUUUUCGCGUUAGAUUUCUUUUAGUUUUCUUUCAACUAGCAAGCUUUUUGUUCUCGCCGCCGCC | |
| 3 | GGAAAUCGCAAAAUUUUUGCUCUUUUUCGCGUUAGAUUUCUUUUAGUUUUCUYKCAACUAGCAAGCUUUUUGUUCUCGCCRCC | |
| 4 | GGAAAUCCCCACAACCGCCUCAUAUCCAGGCUCAAGAAUAGAGCUCAGUGUUUUGUUGUUUAAUCAUUCCGACGUGUUUUGCGAUAUUC | |
| | GCGCAAAGCAGCCAGUCGCGCGCUUGCUUUUAAGUAGAGUUGUUUUUCCACCCGUUUGCCAGGCAUCUUUAAUUUAACAUAUUUUUAUU | |
| | UUUCAGGCUAACCUACGCCGCCACC | |
| 5 | GGAAAUAAGAGAGAAAAGAAGAGUAAGAAGAAAUAUAAGAUCUCCCUGAGCUUCAGGGAGCCCCGGCGCCGCCACC | |
| 6 | GGAAACCCCCCACCCCCGUAAGAGAGAAAAGAAGAGUAAGAAGAAAUAUAAGAUCUCCCUGAGCUUCAGGGAGCCCCGGCGCCGCCACC | |
| 7 | GGAGAACUUCCGCUUCCGUUGGCGCAAGCGCUUUCAUUUUUUCUGCUACCGUGACUAAG | |
| 8 | GGAAAUAAGAGAGAAAAGAAGAGUAAGAAGAAAUAUAAGAGCCACC | |
| 9 | GGAAAUAAGAGAGAAAAGAAGAGUAAGAAGAAAUAUAAGACCCCGGCGCCGCCACC | |
| 10 | GGAAACUUUAUUUAGUGUUACUUUAUUUUCUGUUUAUUUGUGUUUCUUCAGUGGGUUUGUUCUAAUUUCCUUGGCCGCC | |
| 11 | GGAAAAUCUGUAUUAGGUUGGCGUGUUCUUUGGUCGGUUGUUAGUAUUGUUGUUGAUUCGUUUGUGGUCGGUUGCCGCC | |
| 12 | GGAAAAUUAUUAACAUCUUGGUAUUCUCGAUAACCAUUCGUUGGAUUUUAUUGUAUUCGUAGUUUGGGUUCCUGCCGCC | |
| 13 | GGAAAUUAUUAUUAUUUCUAGCUACAAUUUAUCAUUGUAUUAUUUUAGCUAUUCAUCAUUAUUUACUUGGUGAUCAACA | |
| 14 | GGAAAUAGGUUGUUAACCAAGUUCAAGCCUAAUAAGCUUGGAUUCUGGUGACUUGCUUCACCGUUGGCGGGCACCGAUC | |
| 15 | GGAAAUCGUAGAGAGUCGUACUUAGUACAUAUCGACUAUCGGUGGACACCAUCAAGAUUAUAAACCAGGCCAGA | |
| 16 | GGAAACCCGCCCAAGCGACCCCAACAUAUCAGCAGUUGCCCAAUCCCAACUCCCAACACAAUCCCCAAGCAACGCCGCC | |
| 17 | GGAAAGCGAUUGAAGGCGUCUUUUCAACUACUCGAUUAAGGUUGGGUAUCGUCGUGGGACUUGGAAAUUUGUUGUUUCC | |
| 18 | GGAAACUAAUCGAAAUAAAAGAGCCCCGUACUCUUUUAUUUCUAUUAGGUUAGGAGCCUUAGCAUUUGUAUCUUAGGUA | |
| 19 | GGAAAUGUGAUUUCCAGCAACUUCUUUUGAAUAUAUUGAAUUCCUAAUUCAAAGCGAACAAAUCUACAAGCCAUAUACC | |
| 20 | GGAAAUCGUAGAGAGUCGUACUUACGUGGUCGCCAUUGCAUAGCGCGCGAAAGCAACAGGAACAAGAACGCGCC | |
| 21 | GGAAAUCGUAGAGAGUCGUACUUAGAAUAAACAGAGUCGGGUCGACUUGUCUCUGAUACUACGACGUCACAAUC | |
| 22 | GGAAAAUUUGCCUUCGGAGUUGCGUAUCCUGAACUGCCCAGCCUCCUGAUAUACAACUGUUCCGCUUAUUCGGGCCGCC | |
| 23 | GGAAAUCUGAGCAGGAAUCCUUUGUGCAUUGAAGACUUUAGAUUCCUCUCUGCGGUAGACGUGCACUUAUAAGUAUUUG | |
| 24 | GGAAAGCGAUUGAAGGCGUCUUUUCAACUACUCGAUUAAGGUUGGGUAUCGUCGUGGGACUUGGAAAUUUGUUGCCACC | |
| 25 | GGAAAAUUUUAGCCUGGAACGUUAGAUAACUGUCCUGUUGUCUUUAUAUACUUGGUCCCCAAGUAGUUUGUCUUCCAAA | |
| 26 | GGAAAUUUUUUUUUGAUAUUAUAAGAGUUUUUUUUUGAUAUUAAGAAAAUUUUUUUUUGAUAUUAGAAGAGUAAGAAGAAAUAUAAGACCCC | |
| | GGCGCCGCCACC | |
| 27 | GGAAAUAAGAGAGAAAAGAAGAGUAAGAAGAAAUAUAAGAGCCAAAAAAAAAAAACC | |
| 28 | GGAAAUCUCCCUGAGCUUCAGGGAGUAAGAGAGAAAAGAAGAGUAAGAAGAAAUAUAAGACCCCGGCGCCGCCACC | |
| 29 | GGGAUCCUACC | |
| 30 | GGGAAAUAAGAGAGAAAAGAAGAGUAAGAAGAAAUAUAAGAGCCACC | |
| 31 | UAAAGCUCCCCGGGG | |
| 32 | UAAGCCCCUCCGGGG | |
| 33 | LEPGEKPYKCPECGKSFSQSGALTRHQRTHTR | |
| 34 | TCGGGATCTGAGACGCCTGGGACCTCGGAATCGGCTACGCCCGAAAGT | |
| 35 | LEPGEKPYKCPECGKSFSQSGALTRHQRTHTRLEPGEKPYKCPECGKSFSQSGALTRHQRTHTR | |
| 36 | LEPGEKPYKCPECGKSFSQSGALTRHQRTHTRLEPGEKPYKCPECGKSFSQSGALTRHQRTHTRLEPGEKPYKCPECGKSFSQSGALTRHQRTHTR | |
| 37 | PKKKRKVEASSPKKRKVEAS | |
| 38 | KRPAATKKAGQAKKKK | |
| 39 | RQRRNELKRSP | |
| 40 | NQSSNFGPMKGGNFGGRSSGPYGGGGQYFAKPRNQGGY | |
| 41 | RMRIZFKNKGKDTAELRRRRVEVSVELRKAKKDEQILKRRNV | |
| 42 | SALIKKKKKMAP | |
| 43 | RKLKKKIKKL | |
| 44 | REKKKFLKRR | |
| 45 | KRKGDEVDGVDEVAKKKSKK | |
| 46 | RKCLQAGMNLEARKTKK | |
| 47 | GGGAAAUAAGAGAGAAAAGAAGAGUAAGAAGAAAUAUAAGACCCCGGCGCCGCCACCAUGCCUAAGAAGAAGCGCAAGGUGUCUAACCUCC | |
| | UGACUGUGCACCAGAACUUGCCAGCCUUGCCGGUGGACGCAACUUCCGACGAAGUCCGGAAGAACCUCAUGGACAUGUUCCGGGACAGA | |
| | CAGGCGUUUUCAGAGCACACCUGGAAAAUGCUGCUGUCCGUCUGCCGGUCCUGGGCCGCCUGGUGCAAGCUGAACAACAGGAAGUGGU | |
| | UCCCCGCUGAACCGGAGGACGUGCGCGAUUACCUUCUGUACCUUCAAGCCCGGGGACUCGCAGUCAAGACCAUCCAGCAACACCUGGGC | |
| | CAGCUGAACAUGCUGCAUCGGCGGUCCGGACUGCCUAGACCUAGCGAUUCCAAUGCCGUGUCACUCGUGAUGCGGCGGAUCCGGAAGG | |
| | AAAACGUGGACGCCGGGGAAAGGGCCAAGCAGGCACUUGCCUUCGAAAGGACCGACUUCGACCAAGUCCGCUCGCUGAUGGAAAAUUCG | |
| | GACCGGUGUCAGGAUAUUAGGAACCUCGCGUUCCUGGGGAUUGCGUACAAUACCCUGCUGAGAAUCGCCGAGAUUGCCCGCAUCCGCGU | |
| | GAAGGACAUUUCGAGAACCGACGGAGGCCGGAUGCUGAUCCACAUUGGUCGCACCAAGACUCUGGUGUCCACUGCCGGGGUGGAGAAGG | |
| | CCCUGUCGCUGGGCGUGACCAAACUGGUGGAGCGCUGGAUUUCCGUGAGCGGAGUGGCGGAUGACCCGAACAACUACCUGUUCUGCCG | |
| | CGUGCGAAAGAACGGAGUGGCCGCUCCCUCCGCAACGAGCCAGCUCAGCACCAGAGCCUUGGAGGGCAUCUUCGAAGCCACACACCGCC | |
| | UCAUCUAUGGUGCCAAGGACGAUAGCGGCCAGAGAUACCUCGCGUGGUCCGGACAUUCCGCCCGCGUGGGCGCGGCUCGGGACAUGGC | |
| | CAGGGCUGGAGUCUCCAUCCCCGAAAUCAUGCAGGCCGGGGGAUGGACCAACGUGAACAUCGUGAUGAACUACAUCCGGAACCUGGAUU | |
| | CAGAGACUGGUGCCAUGGUCCGCCUGCUGGAGGACGGCGAUUAAUAAUGAAUUAAUAGUGUAGUGACUAGUGACUGACUAGGAUCUGGU | |
| | UACCACUAAACCAGCCUCAAGAACACCCGAAUGGAGUCUCUAAGCUACAUAAUACCAACUUACACUUACAAAAUGUUGUCCCCCAAAAUGUA | |
| | GCCAUUCGUAUCUGCUCCUAAUAAAAAGAAAGUUUCUUCACAUGCGGCCGC | |
| 48 | GGGAAAUAAGAGAGAAAAGAAGAGUAAGAAGAAAUAUAAGACCCCGGCGCCGCCACCAUGGGAGUGAAGGUGCUGUUCGCUCUCAUCUGCA | |
| | UUGCUGUGGCUGAGGCCCAGGUGCAGCUGCAAGAGUCUGGCGGCGGACUGGUGCAACCUGGCGGCAGCUUGAGACUGAGCUGUGCUGC | |
| | CAGCGGCUUCACGCUGGAUUACUACUACAUCGGGUGGUUUAGACAGGCCCCCGGCAAGGAGAGGGAAGCCGUCUCCUGCAUUAGCGGC | |
| | UCAUCCGGGAGCACCUACUACCCGGAUAGCGUCAAGGGCAGAUUCACCAUCUCCCGGGACAACGCCAAGAACACGGUGUACCUGCAGAU | |
| | GAACAGCCUGAAGCCCGAGGACACGGCUGUGUACUACUGCGCUACCAUCAGGAGCAGCAGCUGGGGGGCUGCGUCCAUUACGGAAUGG | |
| | ACUACUGGGGCAAAGGCACCCAGGUGACCGUGUCCAGCGGCACCACCAGACUGCUGUCUGGACACACCUGUUUCACACUGACAGGGCUG | |
| | CUGGGCACCCUGGUGACCAUGGGGCUGCUUACAUAAUGAAUUAAUAGUGUAGUGACUAGUGACUGACUAGGAUCUGGUUACCACUAAACC | |
| | AGCCUCAAGAACACCCGAAUGGAGUCUCUAAGCUACAUAAUACCAACUUACACUUACAAAAUGUUGUCCCCCAAAAUGUAGCCAUUCGUAU | |
| | CUGCUCCUAAUAAAAAGAAAGUUUCUUCACAUGCGGCCG | |
| 49 | c*u*a*ACAGUUGCUUUUAUCACGUUUUAGAgcuagaaauagcAAGUUAAAAUAAGGCUAGUCCGUUAUCaacuugaaaaaguggcaccgagucggugcu | |
| | *u*u*u | |
| 50 | AGGAAAUAAGAGAGAAAAGAAGAGUAAGAAGAAAUAUAAGACCCCGGCGCCGCCACCAUGGACAAGAAAUACUCCAUCGGACUGGACAUCGG | |
| | AACCAACUCCGUGGGAUGGGCCGUGAUUACGGACGAGUACAAAGUGCCUUCUAAAAAGUUCAAAGUGCUCGGCAACACCGACCGGCACUC | |
| | CAUCAAGAAGAACCUCAUUGGAGCACUGCUGUUUGACUCGGGGGAAACUGCUGAAGCGACUCGGCUGAAGAGAACGGCACGCCGCCGGU | |
| | ACACCCGGAGGAAGAAUAGAAUCUGCUACCUUCAAGAAAUCUUUUCCAACGAAAUGGCCAAAGUCGACGACAGCUUCUUCCACCGCCUGG | |
| | AAGAGUCGUUCCUCGUGGAAGAGGACAAGAAGCACGAGAGACA | |
| 51 | u*u*UGAAUuuCUAcUguuGUagaUcCUUGUCaagGcUauUGGU*c*a | |
| 52 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHDNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKD | |
| 53 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKD | |
| 54 | CCTGAATTGCTATGTGTCTGGG | |
| 55 | CTCATACACAACTTTCAGCAGC | |
| 56 | AGTGGGGGTGAATTCAGTGT | |
| 57 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRINKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 58 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFRKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 59 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVRLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 60 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVARHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 61 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRERSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKD | |
| | CSFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKD | |
| | STFYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIA | |
| | LPMGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIP | |
| | LSEIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKN | |
| | KMADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTL | |
| | NLVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYL | |
| | VFKDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYK | |
| | KKWDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFD | |
| | SREEFKKGGDARLPIDCDANGAYHIALKGLYLLLNDFNRDNKGVIQNISNKDWFKFVQEKVYKDGGGGGRSSDDEATADSQHAAPPKKKRKVGGGGKR | |
| | PAATKKAGQAKKKK** | |
| 62 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLRAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 63 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKRGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 64 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVRNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 65 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFERMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 66 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVRRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 67 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLRGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 68 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRRFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTITDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 69 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNRHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 70 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARRNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 71 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDRHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 72 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGRKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 73 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFRQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 74 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGRMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 75 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKRFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 76 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHRTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 77 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLRKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 78 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTITDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIRKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 79 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSRGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 80 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKDK | |
| | KKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKFF | |
| | ANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQILS | |
| | DRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKDC | |
| | SFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKDST | |
| | FYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIALP | |
| | MGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKNK | |
| | MADSISDEAKRLLESGTVVCKVATHDIVKRKHFTENTYLFHCPITMNFKAKDRTNKEFNNHVLEVLNKNPDIKVIGLDRGERHLLYLSLINQKGEIECQKTLN | |
| | LVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYLVF | |
| | KDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYKKK | |
| | WDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFDSR | |
| | EEFKKGGDARLPIDCDANGAYHIALKGLYLLLNDFNRDNKGVIQNISNKDWFKFVQEKVYKDGGGGGRSSDDEATADSQHAAPPKKKRKVGGGGKRPA | |
| | ATKKAGQAKKKK** | |
| 81 | KRPAATKKAGQAKKKK | |
| 82 | RSSDDEATADSQHAAPPKKKRKV | |
| 83 | CTCATACACAACTTTCAGCAGC | |
| 84 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDKRPAATKKAGQAKKKK* | |
| 85 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDGGGGKRPAATKKAGQAKKKK* | |
| 86 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDGGGGPAAKRVKLD* | |
| 87 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTITDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDGGGGKRPAATKKAGQAKKKKGGGGPAAKRVKLD* | |
| 88 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDGGGGPAAKRVKLDGGGGGRSSDDEATADSQHAAPPKKKRKV* | |
| 89 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDGGGGKRPAATKKAGQAKKKKGGGGPAAKRVKLDGGGGPAAKRVKLD* | |
| 90 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDGGGGGRSSDDEATADSQHAAPPKKKRKV* | |
| 91 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDGGGGGRSSDDEATADSQHAAPPKKKRKVGGGGKRPAATKKAGQAKKKK* | |
| 92 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKDLEKLQ | |
| | SGARKKIVEAFKKNPDFEKLFKEGLFKELLPELIKSAPVDEIAVKTKALECFNRFSTYFTGFHRNRKNMYSEEAKSTAISYRIVNENFPKFFANIKLFNYLKEHFP | |
| | RIIIDTEESLKDYLKGKKLDSVFSIDGFNSVLAQSGIDFYNTVIGGISGEAGTKKTQGLNEKINLARQQLSKEEKNKLRGKMVVLFKQILSDRETSSFIPVGFAN | |
| | KEEVYSTVKEFNNSIAEKAVSKVRDLFLHREEFTLNEIFVPAKSLTDFSQAIFGSWSILSEGLFLLEKDSMKKALSESQEEKINKEIAKKDCSFTELQLAYERYCT | |
| | EHNLPVEKFCKDYFDIVDYRGNGAKSEKTKVSILSEILETFLQLDFDHIQDLQQEKNAAIPIKAYLDEVQNLYHHLKLVDYRGEEQKDSTFYSKHDEILTDLSQ | |
| | IVPLYNKVRNFVTKKLGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPLSEIDKMIDEGKLFLFQ | |
| | IYNKDFAKKATGKKNLHTLYWENLFSVENLQDVVLKLNGEAELFWREASIKKDKVIVHKKGSILVNRTTTDGKSIPEAIYQEIYQLKNKMADSISDEAKRLLES | |
| | GTVVCKVATHDIVKDKHFTENTYLFHCPITMNFKAKDRTNKEFNNHVLEVLNKNPDIKVIGLDRGERHLLYLSLINQKGEIECQKTLNLVEQVRNDKTVSVN | |
| | YHEKLVHKEGSRDAARKNWQTIGNIKELKEGYLSAVVHEIASLMVKHNAIVVMEDLNFGFKRGRFAVERQIYQKFENMLIEKLNYLVFKDRKVTEPGGVLNA | |
| | YQLANKSAKVTDVYKQCGWLFYIPAAYTSKIDPRTGFANLFITKGLTNVEKKKEFFGKFDSIRYDATESCFVFSFDYAKICDNADYKKKWDVYTRGTRLVYNK | |
| | TERKNVSVNPTEELQCVFDEFGIKWNTGEDLIESISLIPAEKSNAKFFDVLLRMFNATLQMRNSVPNTDTDYLVSPVKAEDGSFFDSREEFKKGGDARLPID | |
| | CDANGAYHIALKGLYLLLNDFNRDNKGVIQNISNKDWFKFVQEKVYKDGGGGKRPAATKKAGQAKKKKGGGGGRSSDDEATADSQHAAPPKKKRKV* | |
| 93 | MPAAKRVKLDGGGGARIIDEFCGQMNGYSRSITLRNRLVPIGKTEENLKQFLEKDLERATAYPDIKNLIDAIHRNVIEDTLSKVALNWNEIFNILATYQNEKD | |
| | KKKKAAIKKDLEKLQSGARKKIVEAFKKNPDFEKLFKEGLFKELLPELIKSAPVDEIAVKTKALECFNRFSTYFTGFHRNRKNMYSEEAKSTAISYRIVNENFPKF | |
| | FANIKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQIL | |
| | SDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINKEIAKKD | |
| | CSFTELQLAYERYCTEHNLPVEKFCKDYFDIVDYRGNGAKSEKTKVSILSEILETFLQLDFDHIQDLQQEKNAAIPIKAYLDEVQNLYHHLKLVDYRGEEQKD | |
| | STFYSKHDEILTDLSQIVPLYNKVRNFVTKKLGESKKIKLNFDCPTLANGWDENQESSNDAIILRKDGKYYLGIYNPNNKPKFAKKDSIVGDCYEKMAYKQIA | |
| | LPMGLGAFVRKCFGTAQKYGWGCPENCLNSEGKIIIKDEEAKGNLEAIIDCYKDFLNKYEKDGFKYKDYNFSFLDSASYEKLSDFFNDVKPQGYKLSFTSIP | |
| | LSEIDKMIDEGKLFLFQIYNKDFAKKATGKKNLHTLYWENLFSVENLQDVVLKLNGEAELFWREASIKKDKVIVHKKGSILVNRTTTDGKSIPEAIYQEIYQLKN | |
| | KMADSISDEAKRLLESGTVVCKVATHDIVKDKHFTENTYLFHCPITMNFKAKDRTNKEFNNHVLEVLNKNPDIKVIGLDRGERHLLYLSLINQKGEIECQKTL | |
| | NLVEQVRNDKTVSVNYHEKLVHKEGSRDAARKNWQTIGNIKELKEGYLSAVVHEIASLMVKHNAIVVMEDLNFGFKRGRFAVERQIYQKFENMLIEKLNYL | |
| | VFKDRKVTEPGGVLNAYQLANKSAKVTDVYKQCGWLFYIPAAYTSKIDPRTGFANLFITKGLTNVEKKKEFFGKFDSIRYDATESCFVFSFDYAKICDNADYK | |
| | KKWDVYTRGTRLVYNKTERKNVSVNPTEELQCVFDEFGIKWNTGEDLIESISLIPAEKSNAKFFDVLLRMFNATLQMRNSVPNTDTDYLVSPVKAEDGSFFD | |
| | SREEFKKGGDARLPIDCDANGAYHIALKGLYLLLNDFNRDNKGVIQNISNKDWFKFVQEKVYKDGGGGGRSSDDEATADSQHAAPPKKKRKVGGGGKR | |
| | PAATKKAGQAKKKK* | |
| 94 | TCTTCCTTGTCAAGGCTATTGGTCAAG | |
| 95 | TTGGCCTTGTCAAGGCTATTGGTCAAG | |
| 96 | MARIIDEFCGQMNGYTRSITLRNRLVPIGKTEENLKQFLEKDLERATAYPDIKNLIDAIHRNVIEDTLSKVALNWNEIFNILATYQNEKDKKKKAAIKKD | S16T |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 97 | MARIIDEFCGQMNGYSRSITLRNRLVPIGKTEENLKQFLEKDLERATAYPDIKNLIDAIHRNVIEDTLSKVALNWNEIFNILATYQNEKDKKKKAATKK | 197T |
| | DLEKLQSGARKKIVEAFKKNPDFEKLFKEGLFKELLPELIKSAPVDEIAVKTKALECFNRFSTYFTGFHDNRKNMYSEEAKSTAISYRIVNENFPKFFAN | |
| | IKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQ | |
| | ILSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 98 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | R212L |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPLIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 99 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | K127R |
| | LEKLQSGARKKIVEAFKKNPDFEKLFREGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 100 | MARIIDEFCGQMNGYSRSITLRNRLVPIGKTEENLKQFLEKDLERATAYPDIKNLIDAIHRNVIEDTLSKVALNWDEIFNILATYQNEKDKKKKAAIKKD | N76D |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAWHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 101 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | R304S |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDSETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 102 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | V254M |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTMIGGISGEAGTKKTQGLNEKINLARQQLSKEEKNKLRGKMVVLFKQ | |
| | ILSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 103 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | D231V |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLVSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 104 | MARIIDEFCGQMNGYSRSITLRNRLVPFGKTEENLKQFLEKDLERATAYPDIKNLIDAIHRNVIEDTLSKVALNWNEIFNILATYQNEKDKKKKAAIKK | I28F |
| | DLEKLQSGARKKIVEAFKKNPDFEKLFKEGLFKELLPELIKSAPVDEIAVKTKALECFNRFSTYFTGFHDNRKNMYSEEAKSTAISYRIVNENFPKFFAN | |
| | IKLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQ | |
| | ILSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 105 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | A183T |
| | LEKLQSGARKKIVEAFKKNPDFEKLFKEGLFKELLPELIKSAPVDEIAVKTKALECFNRFSTYFTGFHDNRKNMYSEEAKSTTISYRIVNENFPKFFANIK | |
| | LFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQIL | |
| | SDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKINK | |
| | EIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLKL | |
| | VDYRGEEQKDSTFYSKHDEILTDLSQIVPLYNKVRNFVTKKLGESKKIKLNFDCPTLANGWDENQESSNDAIILRKDGKYYLGIYNPNNKPKFAKKD | |
| | SIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYEK | |
| | LSDFFNDVKPQGYKLSFTSIPLSEIDKMIDEGKLFLFQIYNKDFAKKATGKKNLHTLYWENLFSVENLQDVVLKLNGEAELFWREASIKKDKVIVHKK | |
| | GSILVNRTTTDGKSIPEAIYQEIYQLKNKMADSISDEAKRLLESGTVVCKVATHDIVKDKHFTENTYLFHCPITMNFKAKDRTNKEFNNHVLEVLNKN | |
| | PDIKVIGLDRGERHLLYLSLINQKGEIECQKTLNLVEQVRNDKTVSVNYHEKLVHKEGSRDAARKNWQTIGNIKELKEGYLSAVVHEIASLMVKHNA | |
| | IVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITKG | |
| | LTNVEKKKEFFGKFDSIRYDATESCFVFSFDYAKICDNADYKKKWDVYTRGTRLVYNKTERKNVSVNPTEELQCVFDEFGIKWNTGEDLIESISLIPAE | |
| | KSNAKFFDVLLRMFNATLQMRNSVPNTDTDYLVSPVKAEDGSFFDSREEFKKGGDARLPIDCDANGAYHIALKGLYLLLNDFNRDNKGVIQNISN | |
| | KDWFKFVQEKVYKD | |
| 106 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | D341V |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRVLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRINKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 107 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | V312E |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPEGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAWHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 108 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | F197V |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFVANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 109 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | K207N |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLNEHFPRIIIDTEESLKDYLKGKKLDSVFSIDGFNSVLAQSGIDFYNTVIGGISGEAGTKKTQGLNEKINLARQQLSKEEKNKLRGKMVVLFKQ | |
| | ILSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 110 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | V294M |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMMVLFKQ | |
| | ILSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRINKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 111 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | R212Q |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPQIIIDTEESLKDYLKGKKLDSVFSIDGFNSVLAQSGIDFYNTVIGGISGEAGTKKTQGLNEKINLARQQLSKEEKNKLRGKMVVLFKQ | |
| | ILSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 112 | MARIIDEFCGQMNGYSRSITLRNRLVPIGKTEENLKQFLEKDLERATAYPDIKNLIDAIHRNVIEDTLSKVALNWNEISNILATYQNEKDKKKKAAIKKD | F79S |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 113 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | Q299E |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKEI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 114 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | V929I |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQIRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 115 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | D1247G |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRINKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCGANGAYHIALKGLYLLLNDFNRDNKGVIQNIS | |
| | NKDWFKFVQEKVYKD | |
| 116 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | T1107S |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDASESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 117 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | K1284R |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFRFVQEKVYKD | |
| 118 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | V929D |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQDRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 119 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | D1192E |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFEVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 120 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | Q1203H |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRINKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLHMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 121 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | I1063N |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRINKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAWHEIASLMVKHN | |
| | AIVVMEDLNFGFKRGRFAVERQIYQKFENMLIEKLNYLVFKDRKVTEPGGVLNAYQLANKSAKVTDVYKQCGWLFYNPAAYTSKIDPRTGFANLFIT | |
| | KGLTNVEKKKEFFGKFDSIRYDATESCFVFSFDYAKICDNADYKKKWDVYTRGTRLVYNKTERKNVSVNPTEELQCVFDEFGIKWNTGEDLIESISLI | |
| | PAEKSNAKFFDVLLRMFNATLQMRNSVPNTDTDYLVSPVKAEDGSFFDSREEFKKGGDARLPIDCDANGAYHIALKGLYLLLNDFNRDNKGVIQNI | |
| | SNKDWFKFVQEKVYKD | |
| 122 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | K933M |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDMTVSVNYHEKLVHKEGSRDAARKNWQTIGNIKELKEGYLSAVVHEIASLMVKH | |
| | NAIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFIT | |
| | KGLTNVEKKKEFFGKFDSIRYDATESCFVFSFDYAKICDNADYKKKWDVYTRGTRLVYNKTERKNVSVNPTEELQCVFDEFGIKWNTGEDLIESISLI | |
| | PAEKSNAKFFDVLLRMFNATLQMRNSVPNTDTDYLVSPVKAEDGSFFDSREEFKKGGDARLPIDCDANGAYHIALKGLYLLLNDFNRDNKGVIQNI | |
| | SNKDWFKFVQEKVYKD | |
| 123 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | N1249S |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDASGAYHIALKGLYLLLNDFNRDNKGVIQNIS | |
| | NKDWFKFVQEKVYKD | |
| 124 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | A1251V |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRINKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAWHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGVYHIALKGLYLLLNDFNRDNKGVIQNIS | |
| | NKDWFKFVQEKVYKD | |
| 125 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | N1264I |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTRLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLIDFNRDNKGVIQNIS | |
| | NKDWFKFVQEKVYKD | |
| 126 | MARIIDEFCGQMNGYSRSITLRNRLVPIGKTEENLKQFLEKDLERATAYPDIKNLIDAIHRNVIEDTLSKVALNWNEIFNILATYQNEKDKKKKAAIKKD | R1138S |
| | LEKLQSGARKKIVEAFKKNPDFEKLFKEGLFKELLPELIKSAPVDEIAVKTKALECFNRFSTYFTGFHDNRKNMYSEEAKSTAISYRIVNENFPKFFANI | |
| | KLFNYLKEHFPRIIIDTEESLKDYLKGKKLDSVFSIDGFNSVLAQSGIDFYNTVIGGISGEAGTKKTQGLNEKINLARQQLSKEEKNKLRGKMVVLFKQI | |
| | LSDRETSSFIPVGFANKEEVYSTVKEFNNSIAEKAVSKVRDLFLHREEFTLNEIFVPAKSLTDFSQAIFGSWSILSEGLFLLEKDSMKKALSESQEEKIN | |
| | KEIAKKDCSFTELQLAYERYCTEHNLPVEKFCKDYFDIVDYRGNGAKSEKTKVSILSEILETFLQLDFDHIQDLQQEKNAAIPIKAYLDEVQNLYHHLK | |
| | LVDYRGEEQKDSTFYSKHDEILTDLSQIVPLYNKVRNFVTKKLGESKKIKLNFDCPTLANGWDENQESSNDAIILRKDGKYYLGIYNPNNKPKFAKK | |
| | DSIVGDCYEKMAYKQIALPMGLGAFVRKCFGTAQKYGWGCPENCLNSEGKIIIKDEEAKGNLEAIIDCYKDFLNKYEKDGFKYKDYNFSFLDSASYE | |
| | KLSDFFNDVKPQGYKLSFTSIPLSEIDKMIDEGKLFLFQIYNKDFAKKATGKKNLHTLYWENLFSVENLQDVVLKLNGEAELFWREASIKKDKVIVHK | |
| | KGSILVNRTTTDGKSIPEAIYQEIYQLKNKMADSISDEAKRLLESGTVVCKVATHDIVKDKHFTENTYLFHCPITMNFKAKDRTNKEFNNHVLEVLNK | |
| | NPDIKVIGLDRGERHLLYLSLINQKGEIECQKTLNLVEQVRNDKTVSVNYHEKLVHKEGSRDAARKNWQTIGNIKELKEGYLSAVVHEIASLMVKHN | |
| | AIVVMEDLNFGFKRGRFAVERQIYQKFENMLIEKLNYLVFKDRKVTEPGGVLNAYQLANKSAKVTDVYKQCGWLFYIPAAYTSKIDPRTGFANLFITK | |
| | GLTNVEKKKEFFGKFDSIRYDATESCFVFSFDYAKICDNADYKKKWDVYTRGTSLVYNKTERKNVSVNPTEELQCVFDEFGIKWNTGEDLIESISLIP | |
| | AEKSNAKFFDVLLRMFNATLQMRNSVPNTDTDYLVSPVKAEDGSFFDSREEFKKGGDARLPIDCDANGAYHIALKGLYLLLNDFNRDNKGVIQNIS | |
| | NKDWFKFVQEKVYKD | |
| 127 | ATGGCTAGGATAATTGATGAATTTTGTGGACAGATGAATGGATACACCCGTTCCATCACGCTGCGTAACCGCCTGGTGCCGATCGGCAAGAC | S16T |
| | CGAGGAAAATCTGAAACAGTTTCTGGAGAAGGACTTAGAGCGCGCAACTGCGTACCCGGACATCAAAAACCTGATTGACGCTATCCATCGT | |
| | AATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 128 | ATGGCTAGGATAATTGATGAATTTTGTGGACAGATGAATGGATACAGCCGTTCCATCACGCTGCGTAACCGCCTGGTGCCGATCGGCAAGA | I97T |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGACCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 129 | ATGGCTAGGATAATTGATGAATTTTGTGGACAGATGAATGGATACAGCCGTTCCATCACGCTGCGTAACCGCCTGGTGCCGATCGGCAAGA | R212L |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCTAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 130 | ATGGCTAGGATAATTGATGAATTTTGTGGACAGATGAATGGATACAGCCGTTCCATCACGCTGCGTAACCGCCTGGTGCCGATCGGCAAGA | K127R |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAGAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 131 | ATGGCTAGGATAATTGATGAATTTTGTGGACAGATGAATGGATACAGCCGTTCCATCACGCTGCGTAACCGCCTGGTGCCGATCGGCAAGA | N76D |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGGACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 132 | ATGGCTAGGATAATTGATGAATTTTGTGGACAGATGAATGGATACAGCCGTTCCATCACGCTGCGTAACCGCCTGGTGCCGATCGGCAAGA | R304S |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATAGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 133 | ATGGCTAGGATAATTGATGAATTTTGTGGACAGATGAATGGATACAGCCGTTCCATCACGCTGCGTAACCGCCTGGTGCCGATCGGCAAGA | V254M |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCATGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 134 | ATGGCTAGGATAATTGATGAATTTTGTGGACAGATGAATGGATACAGCCGTTCCATCACGCTGCGTAACCGCCTGGTGCCGATCGGCAAGA | D231V |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGTTTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 135 | ATGGCTAGGATAATTGATGAATTTTGTGGACAGATGAATGGATACAGCCGTTCCATCACGCTGCGTAACCGCCTGGTGCCGTTCGGCAAGAC | I28F |
| | CGAGGAAAATCTGAAACAGTTTCTGGAGAAGGACTTAGAGCGCGCAACTGCGTACCCGGACATCAAAAACCTGATTGACGCTATCCATCGT | |
| | AATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 136 | ATGGCTAGGATAATTGATGAATTTTGTGGACAGATGAATGGATACAGCCGTTCCATCACGCTGCGTAACCGCCTGGTGCCGATCGGCAAGA | A183T |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCACAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 137 | ATGGCTAGGATAATTGATGAATTTTGTGGACAGATGAATGGATACAGCCGTTCCATCACGCTGCGTAACCGCCTGGTGCCGATCGGCAAGA | D341V |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGTTCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 138 | ATGGCTAGGATAATTGATGAATTTTGTGGACAGATGAATGGATACAGCCGTTCCATCACGCTGCGTAACCGCCTGGTGCCGATCGGCAAGA | V312E |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGAGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 139 | ATGGCTAGGATAATTGATGAATTTTGTGGACAGATGAATGGATACAGCCGTTCCATCACGCTGCGTAACCGCCTGGTGCCGATCGGCAAGA | F197V |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTGTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 140 | ATGGCTAGGATAATTGATGAATTTTGTGGACAGATGAATGGATACAGCCGTTCCATCACGCTGCGTAACCGCCTGGTGCCGATCGGCAAGA | K207N |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAATGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 141 | ATGGCTAGGATAATTGATGAATTTTGTGGACAGATGAATGGATACAGCCGTTCCATCACGCTGCGTAACCGCCTGGTGCCGATCGGCAAGA | V294M |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGATGGTGCTGTTTAAACAGATCTTGTCTGATCGTGAAACCAGCA | |
| | GCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 142 | ATGGCTAGGATAATTGATGAATTTTGTGGACAGATGAATGGATACAGCCGTTCCATCACGCTGCGTAACCGCCTGGTGCCGATCGGCAAGA | R212Q |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCAAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 143 | ATGGCTAGGATAATTGATGAATTTTGTGGACAGATGAATGGATACAGCCGTTCCATCACGCTGCGTAACCGCCTGGTGCCGATCGGCAAGA | F79S |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTCTAATATCCTCGCGACGTACCAGAACGAAAAAGATAAAA | |
| | AGAAAAAGGCGGCGATCAAAAAGGACCTGGAGAAGCTCCAAAGCGGTGCACGTAAAAAGATTGTTGAGGCATTCAAAAAGAACCCGGATTT | |
| | TGAAAAACTGTTCAAAGAGGGTCTGTTTAAAGAGTTGCTGCCTGAACTGATCAAAAGCGCACCGGTTGATGAAATCGCGGTTAAAACCAAGG | |
| | CGCTGGAATGCTTCAACCGTTTTAGCACCTATTTCACGGGTTTCCACGACAATCGCAAAAACATGTACTCGGAAGAAGCTAAATCTACCGCA | |
| | ATTAGCTATCGCATCGTGAACGAAAATTTCCCGAAATTTTTTGCCAATATCAAGTTGTTCAACTATCTGAAAGAGCACTTTCCGCGAATCATCAT | |
| | TGATACCGAAGAGTCCCTGAAAGACTATTTGAAGGGCAAAAAGCTGGATTCTGTATTTAGCATTGACGGCTTTAACAGCGTGCTGGCGCAGA | |
| | GCGGGATTGATTTCTATAACACCGTGATAGGCGGTATTTCAGGTGAGGCCGGCACCAAAAAAACGCAGGGTCTTAACGAGAAGATTAACTTA | |
| | GCGCGCCAGCAATTATCCAAGGAGGAGAAGAACAAGCTGCGCGGTAAAATGGTGGTGCTGTTTAAACAGATCTTGTCTGATCGTGAAACCA | |
| | GCAGCTTTATCCCAGTGGGTTTTGCCAACAAGGAGGAGGTGTACTCTACGGTGAAAGAATTCAACAACTCGATTGCCGAAAAGGCGGTGTC | |
| | CAAGGTGCGTGATCTATTCCTGCACCGCGAAGAATTCACGCTAAACGAGATTTTCGTCCCGGCAAAGAGCTTGACCGACTTCAGTCAGGCC | |
| | ATCTTCGGATCCTGGTCTATTTTGAGTGAGGGCCTGTTCCTGCTGGAGAAGGACAGCATGAAAAAGGCTCTAAGCGAATCCCAAGAAGAAAA | |
| | GATCAATAAGGAGATTGCGAAAAAAGACTGCTCCTTTACCGAACTACAACTGGCGTACGAGCGCTACTGCACCGAGCACAACTTGCCGGTT | |
| | GAAAAATTCTGCAAAGACTACTTCGACATCGTCGATTACAGGGGAAACGGTGCGAAAAGCGAAAAAACAAAAGTGAGCATCCTGAGCGAAAT | |
| | CCTGGAAACCTTTTTACAATTGGACTTTGACCACATTCAGGACCTGCAACAAGAAAAGAACGCTGCGATTCCTATCAAAGCATATCTTGACGA | |
| | AGTTCAGAATTTGTACCACCACCTGAAGTTGGTCGATTATCGTGGCGAAGAGCAGAAAGACTCAACCTTTTATAGCAAACACGATGAAATCCT | |
| | GACCGATCTTTCTCAGATCGTCCCATTGTATAATAAAGTCCGTAACTTTGTTACCAAAAAACTGGGTGAGAGCAAAAAGATCAAGCTGAACTTC | |
| | GATTGCCCGACTCTGGCTAATGGCTGGGATGAGAATCAAGAGTCCAGCAACGATGCAATTATCCTGAGAAAGGACGGCAAATACTATCTGG | |
| | GCATTTACAACCCGAACAACAAACCGAAATTCGCGAAGAAGGACAGCATCGTGGGTGATTGCTATGAAAAAATGGCATACAAGCAAATCGC | |
| | ACTGCCGATGGGTTTGGGTGCATTTGTGCGCAAATGCTTTGGCACCGCTCAGAAATACGGTTGGGGTTGCCCGGAGAATTGCCTGAACAGC | |
| | GAGGGTAAAATTATCATCAAAGACGAGGAGGCGAAGGGTAATCTGGAGGCAATCATCGACTGTTACAAAGATTTTCTGAACAAGTACGAAAA | |
| | GGACGGCTTCAAGTACAAGGACTACAATTTTTCCTTCCTGGACTCTGCCAGCTATGAAAAACTGTCTGATTTCTTCAACGACGTTAAACCGCA | |
| | GGGCTATAAGCTGAGCTTTACGTCCATCCCACTGTCAGAGATCGATAAGATGATTGATGAAGGTAAACTGTTTTTGTTTCAGATATATAACAAAG | |
| | ACTTCGCGAAGAAGGCGACGGGGAAGAAGAACCTGCATACCCTTTATTGGGAAAACCTATTCTCAGTCGAGAATCTGCAAGATGTGGTGTTG | |
| | AAGCTCAATGGCGAAGCCGAGCTGTTCTGGCGTGAGGCTAGCATCAAAAAGGACAAGGTGATCGTTCACAAAAAAGGTTCTATTCTGGTGAA | |
| | TCGTACCACCACGGATGGTAAGAGCATCCCGGAGGCCATTTATCAAGAAATCTATCAATTAAAAAACAAAATGGCGGATTCCATCAGCGACG | |
| | AAGCGAAACGCCTTCTAGAGTCCGGCACCGTTGTCTGTAAAGTCGCCACTCATGATATCGTTAAGGATAAACATTTTACTGAAAACACGTATC | |
| | TGTTCCATTGTCCGATTACCATGAACTTCAAGGCAAAAGACCGTACCAATAAGGAATTTAACAATCATGTTCTGGAGGTTCTTAACAAGAATCC | |
| | GGATATTAAGGTGATCGGCTTAGACCGTGGCGAACGTCATCTGCTGTACCTGAGCCTGATCAATCAGAAGGGTGAAATTGAGTGCCAGAAAA | |
| | CGCTGAACCTAGTTGAGCAAGTTCGTAATGATAAGACCGTTTCCGTTAATTATCATGAAAAACTGGTGCACAAAGAAGGCAGCCGTGATGCTG | |
| | CGAGAAAAAACTGGCAGACCATTGGCAATATTAAAGAGCTGAAGGAGGGCTACCTGTCGGCGGTGGTGCACGAGATTGCGAGTCTGATGG | |
| | TAAAGCACAATGCGATCGTTGTAATGGAAGACCTGAATTTCGGCTTTAAGCGCGGTCGTTTTGCTGTGGAGCGTCAGATCTACCAAAAGTTCG | |
| | AAAACATGCTGATTGAGAAGCTGAACTATCTGGTTTTTAAAGACCGTAAAGTGACGGAGCCGGGTGGCGTTCTGAACGCGTACCAGTTGGCG | |
| | AACAAGAGCGCGAAGGTGACAGATGTTTATAAACAATGCGGTTGGCTGTTCTATATCCCGGCAGCGTACACCAGCAAAATTGATCCGCGTAC | |
| | CGGTTTCGCGAATCTGTTCATTACCAAGGGTCTGACCAATGTTGAGAAGAAGAAGGAGTTTTTCGGCAAGTTCGACTCGATTCGTTATGATGC | |
| | GACCGAGTCTTGTTTCGTTTTTTCCTTCGACTACGCAAAGATTTGTGACAATGCTGATTACAAGAAGAAATGGGATGTTTACACCCGTGGTACT | |
| | CGTCTCGTTTACAACAAGACCGAACGCAAGAACGTGAGCGTCAACCCAACCGAAGAGTTGCAGTGTGTTTTTGATGAGTTCGGCATCAAGTG | |
| | GAATACCGGCGAAGACCTGATTGAATCTATTAGCCTGATCCCGGCGGAAAAGTCCAACGCGAAGTTCTTCGACGTTTTGTTGCGTATGTTTAA | |
| | CGCGACTCTTCAGATGAGAAACAGCGTGCCGAATACCGACACCGACTACTTGGTCAGCCCGGTGAAAGCTGAAGACGGCAGCTTTTTCGA | |
| | CAGCCGCGAGGAGTTTAAGAAAGGTGGCGATGCCCGCCTGCCGATAGACTGCGATGCTAATGGTGCGTACCATATTGCCCTGAAAGGCTT | |
| | GTACTTGTTGTTGAATGATTTCAACCGTGATAACAAGGGCGTCATCCAAAACATCTCTAACAAGGACTGGTTTAAATTCGTGCAGGAGAAAGTT | |
| | TACAAAGACTAA | |
| 144 | ATGGCTAGGATAATTGATGAATTTTGTGGACAGATGAATGGATACAGCCGTTCCATCACGCTGCGTAACCGCCTGGTGCCGATCGGCAAGA | Q299E |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAAGAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 145 | ATGGCTAGGATAATTGATGAATTTTGTGGACAGATGAATGGATACAGCCGTTCCATCACGCTGCGTAACCGCCTGGTGCCGATCGGCAAGA | V929I |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAATTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 146 | ATGGCTAGGATAATTGATGAATTTTGTGGACAGATGAATGGATACAGCCGTTCCATCACGCTGCGTAACCGCCTGGTGCCGATCGGCAAGA | D1247G |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGGTGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 147 | ATGGCTAGGATAATTGATGAATTTTGTGGACAGATGAATGGATACAGCCGTTCCATCACGCTGCGTAACCGCCTGGTGCCGATCGGCAAGA | T1107S |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGTC | |
| | CGAGTCTTGTTTCGTTTTTTCCTTCGACTACGCAAAGATTTGTGACAATGCTGATTACAAGAAGAAATGGGATGTTTACACCCGTGGTACTCGTC | |
| | TCGTTTACAACAAGACCGAACGCAAGAACGTGAGCGTCAACCCAACCGAAGAGTTGCAGTGTGTTTTTGATGAGTTCGGCATCAAGTGGAAT | |
| | ACCGGCGAAGACCTGATTGAATCTATTAGCCTGATCCCGGCGGAAAAGTCCAACGCGAAGTTCTTCGACGTTTTGTTGCGTATGTTTAACGC | |
| | GACTCTTCAGATGAGAAACAGCGTGCCGAATACCGACACCGACTACTTGGTCAGCCCGGTGAAAGCTGAAGACGGCAGCTTTTTCGACAG | |
| | CCGCGAGGAGTTTAAGAAAGGTGGCGATGCCCGCCTGCCGATAGACTGCGATGCTAATGGTGCGTACCATATTGCCCTGAAAGGCTTGTA | |
| | CTTGTTGTTGAATGATTTCAACCGTGATAACAAGGGCGTCATCCAAAACATCTCTAACAAGGACTGGTTTAAATTCGTGCAGGAGAAAGTTTAC | |
| | AAAGACTAA | |
| 148 | ATGGCTAGGATAATTGATGAATTTTGTGGACAGATGAATGGATACAGCCGTTCCATCACGCTGCGTAACCGCCTGGTGCCGATCGGCAAGA | K1284R |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAGATTCGTGCAGGAGAAAGTTTA | |
| | CAAAGACTAA | |
| 149 | ATGGCTAGGATAATTGATGAATTTTGTGGACAGATGAATGGATACAGCCGTTCCATCACGCTGCGTAACCGCCTGGTGCCGATCGGCAAGA | V929D |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGATCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 150 | ATGGCTAGGATAATTGATGAATTTTGTGGACAGATGAATGGATACAGCCGTTCCATCACGCTGCGTAACCGCCTGGTGCCGATCGGCAAGA | D1192E |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGAGGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 151 | ATGGCTAGGATAATTGATGAATTTTGTGGACAGATGAATGGATACAGCCGTTCCATCACGCTGCGTAACCGCCTGGTGCCGATCGGCAAGA | Q1203H |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCATATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 152 | ATGGCTAGGATAATTGATGAATTTTGTGGACAGATGAATGGATACAGCCGTTCCATCACGCTGCGTAACCGCCTGGTGCCGATCGGCAAGA | I1063N |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATAACCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 153 | ATGGCTAGGATAATTGATGAATTTTGTGGACAGATGAATGGATACAGCCGTTCCATCACGCTGCGTAACCGCCTGGTGCCGATCGGCAAGA | K933M |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATATGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 154 | ATGGCTAGGATAATTGATGAATTTTGTGGACAGATGAATGGATACAGCCGTTCCATCACGCTGCGTAACCGCCTGGTGCCGATCGGCAAGA | N1249S |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTTAAGAAAGGTGGCGATGCCCGCCTGCCGATAGACTGCGATGCTAGTGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 155 | ATGGCTAGGATAATTGATGAATTTTGTGGACAGATGAATGGATACAGCCGTTCCATCACGCTGCGTAACCGCCTGGTGCCGATCGGCAAGA | A12 |
| | CCGAGGAAAATCTGAAACAGTTTCTGGAGAAGGACTTAGAGCGCGCAACTGCGTACCCGGACATCAAAAACCTGATTGACGCTATCCATCG | 51V |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGTGTACCATATTGCCCTGAAAGGCTTGTA | |
| | CTTGTTGTTGAATGATTTCAACCGTGATAACAAGGGCGTCATCCAAAACATCTCTAACAAGGACTGGTTTAAATTCGTGCAGGAGAAAGTTTAC | |
| | AAAGACTAA | |
| 156 | ATGGCTAGGATAATTGATGAATTTTGTGGACAGATGAATGGATACAGCCGTTCCATCACGCTGCGTAACCGCCTGGTGCCGATCGGCAAGA | N1264I |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTCGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGATTGATTTCAACCGTGATAACAAGGGCGTCATCCAAAACATCTCTAACAAGGACTGGTTTAAATTCGTGCAGGAGAAAGTTTA | |
| | CAAAGACTAA | |
| 157 | ATGGCTAGGATAATTGATGAATTTTGTGGACAGATGAATGGATACAGCCGTTCCATCACGCTGCGTAACCGCCTGGTGCCGATCGGCAAGA | R1138S |
| | CCGAGGAAAATCTGAAACAGTTTCTGGAGAAGGACTTAGAGCGCGCAACTGCGTACCCGGACATCAAAAACCTGATTGACGCTATCCATCG | |
| | TAATGTCATTGAGGACACCTTAAGCAAAGTGGCCCTGAACTGGAACGAAATCTTTAATATCCTCGCGACGTACCAGAACGAAAAAGATAAAAA | |
| | GAAAAAGGCGGCGATCAAAAAGGACCTGGAGAAGCTCCAAAGCGGTGCACGTAAAAAGATTGTTGAGGCATTCAAAAAGAACCCGGATTTT | |
| | GAAAAACTGTTCAAAGAGGGTCTGTTTAAAGAGTTGCTGCCTGAACTGATCAAAAGCGCACCGGTTGATGAAATCGCGGTTAAAACCAAGGC | |
| | GCTGGAATGCTTCAACCGTTTTAGCACCTATTTCACGGGTTTCCACGACAATCGCAAAAACATGTACTCGGAAGAAGCTAAATCTACCGCAAT | |
| | TAGCTATCGCATCGTGAACGAAAATTTCCCGAAATTTTTTGCCAATATCAAGTTGTTCAACTATCTGAAAGAGCACTTTCCGCGAATCATCATTG | |
| | ATACCGAAGAGTCCCTGAAAGACTATTTGAAGGGCAAAAAGCTGGATTCTGTATTTAGCATTGACGGCTTTAACAGCGTGCTGGCGCAGAGC | |
| | GGGATTGATTTCTATAACACCGTGATAGGCGGTATTTCAGGTGAGGCCGGCACCAAAAAAACGCAGGGTCTTAACGAGAAGATTAACTTAGC | |
| | GCGCCAGCAATTATCCAAGGAGGAGAAGAACAAGCTGCGCGGTAAAATGGTGGTGCTGTTTAAACAGATCTTGTCTGATCGTGAAACCAGC | |
| | AGCTTTATCCCAGTGGGTTTTGCCAACAAGGAGGAGGTGTACTCTACGGTGAAAGAATTCAACAACTCGATTGCCGAAAAGGCGGTGTCCAA | |
| | GGTGCGTGATCTATTCCTGCACCGCGAAGAATTCACGCTAAACGAGATTTTCGTCCCGGCAAAGAGCTTGACCGACTTCAGTCAGGCCATC | |
| | TTCGGATCCTGGTCTATTTTGAGTGAGGGCCTGTTCCTGCTGGAGAAGGACAGCATGAAAAAGGCTCTAAGCGAATCCCAAGAAGAAAAGAT | |
| | CAATAAGGAGATTGCGAAAAAAGACTGCTCCTTTACCGAACTACAACTGGCGTACGAGCGCTACTGCACCGAGCACAACTTGCCGGTTGAA | |
| | AAATTCTGCAAAGACTACTTCGACATCGTCGATTACAGGGGAAACGGTGCGAAAAGCGAAAAAACAAAAGTGAGCATCCTGAGCGAAATCCT | |
| | GGAAACCTTTTTACAATTGGACTTTGACCACATTCAGGACCTGCAACAAGAAAAGAACGCTGCGATTCCTATCAAAGCATATCTTGACGAAGTT | |
| | CAGAATTTGTACCACCACCTGAAGTTGGTCGATTATCGTGGCGAAGAGCAGAAAGACTCAACCTTTTATAGCAAACACGATGAAATCCTGAC | |
| | CGATCTTTCTCAGATCGTCCCATTGTATAATAAAGTCCGTAACTTTGTTACCAAAAAACTGGGTGAGAGCAAAAAGATCAAGCTGAACTTCGAT | |
| | TGCCCGACTCTGGCTAATGGCTGGGATGAGAATCAAGAGTCCAGCAACGATGCAATTATCCTGAGAAAGGACGGCAAATACTATCTGGGCA | |
| | TTTACAACCCGAACAACAAACCGAAATTCGCGAAGAAGGACAGCATCGTGGGTGATTGCTATGAAAAAATGGCATACAAGCAAATCGCACTG | |
| | CCGATGGGTTTGGGTGCATTTGTGCGCAAATGCTTTGGCACCGCTCAGAAATACGGTTGGGGTTGCCCGGAGAATTGCCTGAACAGCGAG | |
| | GGTAAAATTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAACCGCAGGG | |
| | CTATAAGCTGAGCTTTACGTCCATCCCACTGTCAGAGATCGATAAGATGATTGATGAAGGTAAACTGTTTTTGTTTCAGATATATAACAAAGACTT | |
| | CGCGAAGAAGGCGACGGGGAAGAAGAACCTGCATACCCTTTATTGGGAAAACCTATTCTCAGTCGAGAATCTGCAAGATGTGGTGTTGAAG | |
| | CTCAATGGCGAAGCCGAGCTGTTCTGGCGTGAGGCTAGCATCAAAAAGGACAAGGTGATCGTTCACAAAAAAGGTTCTATTCTGGTGAATCG | |
| | TACCACCACGGATGGTAAGAGCATCCCGGAGGCCATTTATCAAGAAATCTATCAATTAAAAAACAAAATGGCGGATTCCATCAGCGACGAAG | |
| | CGAAACGCCTTCTAGAGTCCGGCACCGTTGTCTGTAAAGTCGCCACTCATGATATCGTTAAGGATAAACATTTTACTGAAAACACGTATCTGTT | |
| | CCATTGTCCGATTACCATGAACTTCAAGGCAAAAGACCGTACCAATAAGGAATTTAACAATCATGTTCTGGAGGTTCTTAACAAGAATCCGGAT | |
| | ATTAAGGTGATCGGCTTAGACCGTGGCGAACGTCATCTGCTGTACCTGAGCCTGATCAATCAGAAGGGTGAAATTGAGTGCCAGAAAACGC | |
| | TGAACCTAGTTGAGCAAGTTCGTAATGATAAGACCGTTTCCGTTAATTATCATGAAAAACTGGTGCACAAAGAAGGCAGCCGTGATGCTGCGA | |
| | GAAAAAACTGGCAGACCATTGGCAATATTAAAGAGCTGAAGGAGGGCTACCTGTCGGCGGTGGTGCACGAGATTGCGAGTCTGATGGTAAA | |
| | GCACAATGCGATCGTTGTAATGGAAGACCTGAATTTCGGCTTTAAGCGCGGTCGTTTTGCTGTGGAGCGTCAGATCTACCAAAAGTTCGAAAA | |
| | CATGCTGATTGAGAAGCTGAACTATCTGGTTTTTAAAGACCGTAAAGTGACGGAGCCGGGTGGCGTTCTGAACGCGTACCAGTTGGCGAAC | |
| | AAGAGCGCGAAGGTGACAGATGTTTATAAACAATGCGGTTGGCTGTTCTATATCCCGGCAGCGTACACCAGCAAAATTGATCCGCGTACCG | |
| | GTTTCGCGAATCTGTTCATTACCAAGGGTCTGACCAATGTTGAGAAGAAGAAGGAGTTTTTCGGCAAGTTCGACTCGATTCGTTATGATGCGA | |
| | CCGAGTCTTGTTTCGTTTTTTCCTTCGACTACGCAAAGATTTGTGACAATGCTGATTACAAGAAGAAATGGGATGTTTACACCCGTGGTACTAGT | |
| | CTCGTTTACAACAAGACCGAACGCAAGAACGTGAGCGTCAACCCAACCGAAGAGTTGCAGTGTGTTTTTGATGAGTTCGGCATCAAGTGGAA | |
| | TACCGGCGAAGACCTGATTGAATCTATTAGCCTGATCCCGGCGGAAAAGTCCAACGCGAAGTTCTTCGACGTTTTGTTGCGTATGTTTAACG | |
| | CGACTCTTCAGATGAGAAACAGCGTGCCGAATACCGACACCGACTACTTGGTCAGCCCGGTGAAAGCTGAAGACGGCAGCTTTTTCGACA | |
| | GCCGCGAGGAGTTTAAGAAAGGTGGCGATGCCCGCCTGCCGATAGACTGCGATGCTAATGGTGCGTACCATATTGCCCTGAAAGGCTTGT | |
| | ACTTGTTGTTGAATGATTTCAACCGTGATAACAAGGGCGTCATCCAAAACATCTCTAACAAGGACTGGTTTAAATTCGTGCAGGAGAAAGTTTA | |
| | CAAAGACTAA | |
| 158 | GGATCTCGACGCTCTCCCT | CZ- |
| | | 1824 |
| 159 | TCCCATTCGCCATGCCGAAG | CZ- |
| | | 4750 |
| 160 | CTTGGCCTTGTCAAGGCTATTGGTCAAGA | CZ- |
| | | 4734 |
| 161 | AGCTTCTTGACCAATAGCCTTGACAAGGCCAAGGTAC | CZ- |
| | | 4735 |
| 162 | CTAGTGTAATTTCTACTAAGTGTAGATACCATCAGAGGACACTTGGATTCG | CZ- |
| | | 4402 |
| 163 | CGGCCGAATCCAAGTGTCCTCTGATGGTATCTACACTTAGTAGAAATTACA | CZ- |
| | | 4403 |
| 164 | ATCTACACTTAGTAGAAATTACACTAGCTAGTGTTTAACGCTGGTCTG | CZ- |
| | | 5085 |
| 165 | GGGTCTTGAGGGGTTTTTTGGTACCTTGACAGCTAGCTCAG | CZ- |
| | | 5086 |
| 166 | CTAGTGTAATTTCTACTAAGTGTAGATCCTTGTCAAGGCTATTGGTCAAGG | CZ- |
| | | 4417 |
| 167 | CAAAAAACCCCTCAAGACCC | CZ- |
| | | 3621 |
| 168 | ATCTACAACAGTAGAAATTCACACTAGCTCGAGTTAGTCTTTGTAAACTTTCTC | CZ- |
| | | 5138 |
| 169 | CTAGTGTGAATTTCTACTGTTGTAGATACCATCAGAGGACACTTGGATTCG | CZ- |
| | | 5151 |
| 170 | CGGCCGAATCCAAGTGTCCTCTGATGGTATCTACAACAGTAGAAATTCACA | CZ- |
| | | 5152 |
| 171 | CTAGTGTGAATTTCTACTGTTGTAGATCCTTGTCAAGGCTATTGGTCAAGG | CZ- |
| | | 4415 |
| 172 | ATGGTCTCCCATGGCTAGGATAATTGATGAAT | CZ- |
| | | 5248 |
| 173 | ATGGTCTCGGATCCGAAGATGGCCTG | CZ- |
| | | 5239 |
| 174 | CGTCTCGCATGGTCTCGATATCATGAGTGGCGACTTTACAGA | CZ- |
| | | 5240 |
| 175 | CGTCTCGCATGGTCTCCTCGAGCTAGTGTGAATTTCTACTGTTGTAGAT | CZ- |
| | | 5249 |
| 176 | CGTCTCGCATGGTCTCTGAAGTCGGTCAAGCTCTTTGC | CZ- |
| | | 5669 |
| 177 | CGTCTCGCATGGTCTCTCGCACTGCCGATGGGTTTG | CZ- |
| | | 5670 |
| 178 | CGTCTCGCATGGTCTCGTTGAAGAAATCAGACAGTTTTTCATAGC | CZ- |
| | | 5673 |
| 179 | CGTCTCGCATGGTCTCGTATCTGTTCCATTGTCCGATTACCATG | CZ- |
| | | 5674 |
| 180 | ATGGTCTCGATATCGTTAAGGATAAACATTTTACTGAAAAC | CZ- |
| | | 5250 |
| 181 | ATGGTCTCCTCGAGTTAGTCTTTGTAAACTTTCTC | CZ- |
| | | 5251 |
| 182 | ATTAGGTCTCGTGCGATTTGCTTGTATGCCATTTTTTC | CZ- |
| | | 5672 |
| 183 | ATTAGGTCTCACTTCAGTCAGGCCATCTTCGGATCC | CZ- |
| | | 5760 |
| 184 | ATTAGGTCTCTTCAACGACGTTAAACCGCAGGG | CZ- |
| | | 5675 |
| 185 | ATTAGGTCTCAGATACGTGTTTTCAGTAAAATGTTTATCCTTAACGATATC | CZ- |
| | | 5676 |
| 186 | GTAGAAATAATTTTGTTTAACTTTAATAAGGAGATATACCATGG | CZ- |
| | | 5165 |
| 187 | CCTCACTCAAAATAGACCAGGATCC | CZ- |
| | | 5167 |
| 188 | TCTGTAAAGTCGCCACTCATGATATC | CZ- |
| | | 5170 |
| 189 | ATCTACAACAGTAGAAATTCACACTAGCTCGAG | CZ- |
| | | 5171 |
| 190 | CTCTTCCTTGTCAAGGCTATTGGTCAAGA | CZ- |
| | | 5082 |
| 191 | AGCTTCTTGACCAATAGCCTTGACAAGGAAGAGGTAC | CZ- |
| | | 5083 |
| 192 | UGAAUUUCUACUGUUGUAGAU | |
| 193 | u*g*aauuucuacuguuguagauucuucgcccagagcaucccau*g*g | |
| 194 | u*g*aauuucuacuguuguagauucuUCGcccagaGcaUcccau*g*g | |
| 195 | u*g*AAUuucuacUguuGuagaUucuucgcccagagcaucccau*g*g | |
| 196 | u*g*AAUuucuacUguuGuagaUucuUCGcccagaGcaUcccau*g*g | |
| 197 | u*g*AAUuuCUacUguuGuagaUucuUCGCccaGaGcaUCCcau*g*g | |
| 198 | u*g*AAUuuCUacUguuGuagaUuCUUCGCccaGaGCAUCCcau*g*g | |
| 199 | u*u*UGAAUuuCUacUguuGuagaUuCUUCGCccaGaGCAUCCcau*g*g | |
| 200 | u*g*aauuucuacuguuguagauagugggggugaauucagu*g*u | |
| 201 | u*g*aauuucuacuguuguagauaguGGGggugaaUucAgu*g*u | |
| 202 | u*g*AAUuucuacUguuGuagaUaguGGGggugaaUucAgu*g*u | |
| 203 | u*g*AAUuucuacUguuGuagaUagugggggugaauucagu*g*u | |
| 204 | u*g*AAUuuCUacUguuGuagaUaguGGGGgugAaUucAGU*g*u | |
| 205 | u*g*AAUuuCUacUguuGuagaUaGUGGGGgugAaUUCAGU*g*u | |
| 206 | u*u*UGAAUuuCUAcUguuGUagaUaGUGGGGgugAaUUCAGU*g*u | |
| 207 | u*g*aauuucuacuguuguagauccuugucaaggcuauuggu*c*a | |
| 208 | u*g*aauuucuacuguuguagauccuUGUcaaggcUauUggu*c*a | |
| 209 | u*g*AAUuucuacUguuGuagaUccuugucaaggcuauuggu*c*a | |
| 210 | u*g*AAUuucuacUguuGuagaUccuUGUcaaggcUauUggu*c*a | |
| 211 | u*g*AAUuuCUacUguuGuagaUccuUGUCaagGcUauUGGu*c*a | |
| 212 | u*g*AAUuuCUacUguuGuagaUcCUUGUCaagGcUAUUGGu*c*a | |
| 213 | u*u*UGAAUuuCUAcUguuGUagaUcCUUGUCaagGcUAUUGGu*c*a | |
| 214 | u*g*aauuucuacuguuguagauagugggggugaauucagu*g*u | |
| 215 | u*g*AAUuucuacUguuGuagaUaguGGGggugaaUucAgu*g*u | |
| 216 | u*g*AAUuuCUacUguuGuagaUaguGGGGgugAaUucAGU*g*u | |
| 217 | u*g*AAUuuCUacUguuGuagaUaGUGGGGgugAaUUCAGU*g*u | |
| 218 | u*u*UUAAUuuCUAcUguuGUagaUaGUGGGGgugAaUUCAGU*g*u | |
| 219 | AAAAGCACCGA | |
| 220 | UAUAAACAAUCGUCAUCCUCGUUUUAGAGCUAGAAAUAGCAAGUUAAAAUAAGGCUAGUCCGUUAUCAACUUGAAAAAGUGGCACCGAGUC | |
| | GGUGCUUUU | |
| 221 | GGAUACCAACCCGCGGGGUCGUUUUAGAGCUAGAAAUAGCAAGUUAAAAUAAGGCUAGUCCGUUAUCAACUUGAAAAAGUGGCACCGAGU | |
| | CGGUGCUUUU | |
| 222 | UGUUUAUCAACGUCAUCUAGGUUUUAGAGCUAGAAAUAGCAAGUUAAAAUAAGGCUAGUCCGUUAUCAACUUGAAAAAGUGGCACCGAGUC | |
| | GGUGCUUUU | |
| 223 | AUCUACUUAGAAAGCGAACAGUUUUAGAGCUAGAAAUAGCAAGUUAAAAUAAGGCUAGUCCGUUAUCAACUUGAAAAAGUGGCACCGAGUC | |
| | GGUGCUUUU | |
| 224 | UUGCUUUUAUCACAGGCUCCGUUUUAGAGCUAGAAAUAGCAAGUUAAAAUAAGGCUAGUCCGUUAUCAACUUGAAAAAGUGGCACCGAGUC | |
| | GGUGCUUUU | |
| 225 | CUAACAGUUGCUUUUAUCACGUUUUAGAGCUAGAAAUAGCAAGUUAAAAUAAGGCUAGUCCGUUAUCAACUUGAAAAAGUGGCACCGAGUC | |
| | GGUGCUUUU | |
| 226 | GUCUCGGUGGUGGACUAAACGUUUUAGAGCUAGAAAUAGCAAGUUAAAAUAAGGCUAGUCCGUUAUCAACUUGAAAAAGUGGCACCGAGUC | |
| | GGUGCUUUU | |
| 227 | CUGAAGGGAUACCAACCCGCGUUUUAGAGCUAGAAAUAGCAAGUUAAAAUAAGGCUAGUCCGUUAUCAACUUGAAAAAGUGGCACCGAGUC | |
| | GGUGCUUUU | |
| 228 | UGAACCAGACCACGGCCCGUGUUUUAGAGCUAGAAAUAGCAAGUUAAAAUAAGGCUAGUCCGUUAUCAACUUGAAAAAGUGGCACCGAGUC | |
| | GGUGCUUUU | |
| 229 | AUCACCCGAGUGCCUUUGACGUUUUAGAGCUAGAAAUAGCAAGUUAAAAUAAGGCUAGUCCGUUAUCAACUUGAAAAAGUGGCACCGAGUC | |
| | GGUGCUUUU | |
| 230 | UAAUUUCUACUAAGUGUAGAUCUGGAGCCUGUGAUAAAAGC | |
| 231 | UAAUUUCUACUAAGUGUAGAUCCUUGUCAAGGCUAUUGGUC | |
| 232 | UAAUUUCUACUAAGUGUAGAUCAUUGAGAUAGUGUGGGGAA | |
| 233 | CUUGUCAAGGCUAUUGGUCAGUUUUAGAGCUAGAAAUAGCAAGUUAAAAUAAGGCUAGUCCGUUAUCAACUUGAAAAAGUGGCACCGAGUC | |
| | GGUGCUUUU | |
| 234 | AUUGAGAUAGUGUGGGGAAGGUUUUAGAGCUAGAAAUAGCAAGUUAAAAUAAGGCUAGUCCGUUAUCAACUUGAAAAAGUGGCACCGAGUC | |
| | GGUGCUUUU | |
| 235 | CUUGACCAAUAGCCUUGACAGUUUUAGAGCUAGAAAUAGCAAGUUAAAAUAAGGCUAGUCCGUUAUCAACUUGAAAAAGUGGCACCGAGUC | |
| | GGUGCUUUU | |
| 236 | GUGGGGAAGGGGCCCCCAAGGUUUUAGAGCUAGAAAUAGCAAGUUAAAAUAAGGCUAGUCCGUUAUCAACUUGAAAAAGUGGCACCGAGU | |
| | CGGUGCUUUU | |
| 237 | CAAGGCUAUUGGUCAAGGCAGUUUUAGAGCUAGAAAUAGCAAGUUAAAAUAAGGCUAGUCCGUUAUCAACUUGAAAAAGUGGCACCGAGUC | |
| | GGUGCUUUU | |
| 238 | CUUGUCAAGGCUAUUGGUCGUUUUAGAGCUAGAAAUAGCAAGUUAAAAUAAGGCUAGUCCGUUAUCAACUUGAAAAAGUGGCACCGAGUC | |
| | GGUGCUUUU | |
| 239 | UAGCCUUGACAAGGCAAACUGUUUUAGAGCUAGAAAUAGCAAGUUAAAAUAAGGCUAGUCCGUUAUCAACUUGAAAAAGUGGCACCGAGUC | |
| | GGUGCUUUU | |
| 240 | UAUCUGUCUGAAACGGUCCCGUUUUAGAGCUAGAAAUAGCAAGUUAAAAUAAGGCUAGUCCGUUAUCAACUUGAAAAAGUGGCACCGAGUC | |
| | GGUGCUUUU | |
| 241 | GUGGGGAAGGGGCCCCCAAGUUUUAGAGCUAGAAAUAGCAAGUUAAAAUAAGGCUAGUCCGUUAUCAACUUGAAAAAGUGGCACCGAGUC | |
| | GGUGCUUUU | |
| 242 | AGAUAUUUGCAUUGAGAUAGGUUUUAGAGCUAGAAAUAGCAAGUUAAAAUAAGGCUAGUCCGUUAUCAACUUGAAAAAGUGGCACCGAGUC | |
| | GGUGCUUUU | |
| 243 | GGCAAGGCUGGCCAACCCAUGUUUUAGAGCUAGAAAUAGCAAGUUAAAAUAAGGCUAGUCCGUUAUCAACUUGAAAAAGUGGCACCGAGUC | |
| | GGUGCUUUU | |
| 244 | AGGCAAGGCUGGCCAACCCAGUUUUAGAGCUAGAAAUAGCAAGUUAAAAUAAGGCUAGUCCGUUAUCAACUUGAAAAAGUGGCACCGAGUC | |
| | GGUGCUUUU | |
| 245 | UAAUUUCUACUAAGUGUAGAUCCUUGUCAAGGCUAUUGGUCA | |
| 246 | UAAUUUCUACUAAGUGUAGAUCCUUGUCAAGGCUAUUGGUCAA | |
| 247 | UAAUUUCUACUAAGUGUAGAUCCUUGUCAAGGCUAUUGGUCAAG | |
| 248 | UGAAUUUCUACUGUUGUAGAUCCUUGUCAAGGCUAUUGGUCAAG | |
| 249 | AUAAUUUCUACUGUUGUAGAUCCUUGUCAAGGCUAUUGGUCAAG | |
| 250 | UAAAUUUCUACUAUUGUAGAUCCUUGUCAAGGCUAUUGGUCAAG | |
| 251 | UAAUUUCUACUAAGUGUAGAUCAUUGAGAUAGUGUGGGGAAGGG | |
| 252 | UGAAUUUCUACUGUUGUAGAUCAUUGAGAUAGUGUGGGGAAGGG | |
| 253 | AUAAUUUCUACUGUUGUAGAUCAUUGAGAUAGUGUGGGGAAGGG | |
| 254 | UAAAUUUCUACUAUUGUAGAUCAUUGAGAUAGUGUGGGGAAGGG | |
| 255 | UAAUUUCUACUCUUGUAGAUCCUUGUCAAGGCUAUUGGUC | |
| 256 | UAAUUUCUACUCUUGUAGAUCCUUGUCAAGGCUAUUGGUCA | |
| 257 | UAAUUUCUACUCUUGUAGAUCCUUGUCAAGGCUAUUGGUCAA | |
| 258 | UAAUUUCUACUCUUGUAGAUCCUUGUCAAGGCUAUUGGUCAAG | |
| 259 | UAAUUUCUACUCUUGUAGAUCAUUGAGAUAGUGUGGGGAAGGG | |
| 260 | AATTTCTACTGTTGTAGATccttgtcaaggctattggtcaag | |
| 261 | AAUUUCUACUUCGGUAGAUccuugucaaggcuauuggucaag | |
| 262 | AATTTCTACTATTGTAGATccttgtcaaggctattggtcaag | |
| 263 | UAAUUUCUACUCUUGUAGAUCCCAGGGGGGCCUCUUUCGG | |
| 264 | UAAUUUCUACUCUUGUAGAUUCACAGGCUCCAGGAAGGGU | |
| 265 | UAAUUUCUACUCUUGUAGAUAAGCUAGUCUAGUGCAAGCU | |
| 266 | UAAUUUCUACUCUUGUAGAUCACUGGAAUCAGCUAUCUGC | |
| 267 | UAAUUUCUACUCUUGUAGAUAGCCAUCUCACUACAGAUAA | |
| 268 | UAAUUUCUACUCUUGUAGAUCCCAUGGGGCACAGUCAGGC | |
| 269 | UUUUAUCACAGGCUCCAGGAGUUUUAGAGCUAGAAAUAGCAAGUUAAAAUAAGGCUAGUCCGUUAUCAACUUGAAAAAGUGGCACCGAGUC | |
| | GGUGCUUUU | |
| 270 | UUUAUCACAGGCUCCAGGAAGUUUUAGAGCUAGAAAUAGCAAGUUAAAAUAAGGCUAGUCCGUUAUCAACUUGAAAAAGUGGCACCGAGUC | |
| | GGUGCUUUU | |
| 271 | ACUGAAUCGGAACAAGGCAAGUUUUAGAGCUAGAAAUAGCAAGUUAAAAUAAGGCUAGUCCGUUAUCAACUUGAAAAAGUGGCACCGAGUC | |
| | GGUGCUUUU | |
| 272 | GAUAAACAAUCGUCAUCCUCGUUUUAGAGCUAGAAAUAGCAAGUUAAAAUAAGGCUAGUCCGUUAUCAACUUGAAAAAGUGGCACCGAGUC | |
| | GGUGCUUUU | |
| 273 | GGAUACCAACCCGCGGGGUCGUUUUAGAGCUAGAAAUAGCAAGUUAAAAUAAGGCUAGUCCGUUAUCAACUUGAAAAAGUGGCACCGAGU | |
| | CGGUGCUUUU | |
| 274 | UGUUUAUCAACGUCAUCUAGGUUUUAGAGCUAGAAAUAGCAAGUUAAAAUAAGGCUAGUCCGUUAUCAACUUGAAAAAGUGGCACCGAGUC | |
| | GGUGCUUUU | |
| 275 | AUCUACUUAGAAAGCGAACAGUUUUAGAGCUAGAAAUAGCAAGUUAAAAUAAGGCUAGUCCGUUAUCAACUUGAAAAAGUGGCACCGAGUC | |
| | GGUGCUUUU | |
| 276 | UUGCUUUUAUCACAGGCUCCGUUUUAGAGCUAGAAAUAGCAAGUUAAAAUAAGGCUAGUCCGUUAUCAACUUGAAAAAGUGGCACCGAGUC | |
| | GGUGCUUUU | |
| 277 | CUAACAGUUGCUUUUAUCACGUUUUAGAGCUAGAAAUAGCAAGUUAAAAUAAGGCUAGUCCGUUAUCAACUUGAAAAAGUGGCACCGAGUC | |
| | GGUGCUUUU | |
| 278 | GUCUCGGUGGUGGACUAAACGUUUUAGAGCUAGAAAUAGCAAGUUAAAAUAAGGCUAGUCCGUUAUCAACUUGAAAAAGUGGCACCGAGUC | |
| | GGUGCUUUU | |
| 279 | CUGAAGGGAUACCAACCCGCGUUUUAGAGCUAGAAAUAGCAAGUUAAAAUAAGGCUAGUCCGUUAUCAACUUGAAAAAGUGGCACCGAGUC | |
| | GGUGCUUUU | |
| 280 | UGAACCAGACCACGGCCCGUGUUUUAGAGCUAGAAAUAGCAAGUUAAAAUAAGGCUAGUCCGUUAUCAACUUGAAAAAGUGGCACCGAGUC | |
| | GGUGCUUUU | |
| 281 | AUCACCCGAGUGCCUUUGACGUUUUAGAGCUAGAAAUAGCAAGUUAAAAUAAGGCUAGUCCGUUAUCAACUUGAAAAAGUGGCACCGAGUC | |
| | GGUGCUUUU | |
| 282 | CUUGACCAAUAGCCUUGACAGUUUUAGAGCUAGAAAUAGCAAGUUAAAAUAAGGCUAGUCCGUUAUCAACUUGAAAAAGUGGCACCGAGUC | |
| | GGUGCUUUU | |
| 283 | GUGGGGAAGGGGCCCCCAAGGUUUUAGAGCUAGAAAUAGCAAGUUAAAAUAAGGCUAGUCCGUUAUCAACUUGAAAAAGUGGCACCGAGU | |
| | CGGUGCUUUU | |
| 284 | CUUGACCAAUAGCCUUGACAGUUUUAGAGCUAGAAAUAGCAAGUUAAAAUAAGGCUAGUCCGUUAUCAACUUGAAAAAGUGGCACCGAGUC | |
| | GGUGCUUUU | |
| 285 | GUGGGGAAGGGGCCCCCAAGGUUUUAGAGCUAGAAAUAGCAAGUUAAAAUAAGGCUAGUCCGUUAUCAACUUGAAAAAGUGGCACCGAGU | |
| | CGGUGCUUUU | |
| 286 | CAAGGCUAUUGGUCAAGGCAGUUUUAGAGCUAGAAAUAGCAAGUUAAAAUAAGGCUAGUCCGUUAUCAACUUGAAAAAGUGGCACCGAGUC | |
| | GGUGCUUUU | |
| 287 | CUUGUCAAGGCUAUUGGUCGUUUUAGAGCUAGAAAUAGCAAGUUAAAAUAAGGCUAGUCCGUUAUCAACUUGAAAAAGUGGCACCGAGUC | |
| | GGUGCUUUU | |
| 288 | UAGCCUUGACAAGGCAAACUGUUUUAGAGCUAGAAAUAGCAAGUUAAAAUAAGGCUAGUCCGUUAUCAACUUGAAAAAGUGGCACCGAGUC | |
| | GGUGCUUUU | |
| 289 | UAUCUGUCUGAAACGGUCCCGUUUUAGAGCUAGAAAUAGCAAGUUAAAAUAAGGCUAGUCCGUUAUCAACUUGAAAAAGUGGCACCGAGUC | |
| | GGUGCUUUU | |
| 290 | UGAAUUUCUACUGUUGUAGAUCCUUGUCAAGGCUAUUGGUCA | |
| 291 | AUAAUUUCUACUGUUGUAGAUCCUUGUCAAGGCUAUUGGUCA | |
| 292 | UAAAUUUCUACUAUUGUAGAUCCUUGUCAAGGCUAUUGGUCA | |
| 293 | AAUUUCUACUGUUGUAGAUCCUUGUCAAGGCUAUUGGUCA | |
| 294 | AAUUUCUACUUCGGUAGAUCCUUGUCAAGGCUAUUGGUCA | |
| 295 | AAUUUCUACUAUUGUAGAUCCUUGUCAAGGCUAUUGGUCA | |
| 296 | ACUGAAUCGGAACAAGGCAAGUUUUAGAGCUAGAAAUAGCAAGUUAAAAUAAGGCUAGUCCGUUAUCAACUUGAAAAAGUGGCACCGAGUC | |
| | GGUGCUUUU | |
| 297 | CUUGUCAAGGCUAUUGGUCAGUUUUAGAGCUAGAAAUAGCAAGUUAAAAUAAGGCUAGUCCGUUAUCAACUUGAAAAAGUGGCACCGAGUC | |
| | GGUGCUUUU | |
| 298 | CTAACAGTTGCTTTTATCAC | |
| 299 | ugaauuucuacuguuguagauccuugucaaggcuauugguca | |
| 300 | ugaauuucuacuguuguagauccuUGUcaaggcUauUgguca | |
| 301 | ugAAUuucuacUguuGuagaUccuugucaaggcuauugguca | |
| 302 | ugAAUuucuacUguuGuagaUccuUGUcaaggcUauUgguca | |
| 303 | ugAAUuuCUacUguuGuagaUccuUGUCaagGcUauUGGuca | |
| 304 | ugAAUuuCUacUguuGuagaUcCUUGUCaagGcUAUUGGuca | |
| 305 | uuUGAAUuuCUAcUguuGUagaUcCUUGUCaagGcUAUUGGuca | |
| 306 | UGAAUUUCUACUGUUGUAGAUAUCACAGGCUCCAGGAAGGGU | |
| 307 | UGAAUUUCUACUGUUGUAGAUUCACAGGCUCCAGGAAGGGUU | |
| 308 | UGAAUUUCUACUGUUGUAGAUGCCUCUGAUUAGGGUGGGGGC | |
| 309 | UGAAUUUCUACUGUUGUAGAUCCUUGUCAAGGCUAUUGGUCAAG | |
| 310 | UGAAUUUCUACUGUUGUAGAUCAUUGAGAUAGUGUGGGGAAG | |
| 311 | UGAAUUUCUACUGUUGUAGAUAGACAGAUAUUUGCAUUGAGA | |
| 312 | UGAAUUUCUACUGUUGUAGAUGCCAGGGACCGUUUCAGACAG | |
| 313 | UGAAUUUCUACUGUUGUAGAUCGGCUGGCUAGGGAUGAAGAA | |
| 314 | UGAAUUUCUACUGUUGUAGAUCAUUGAGAUAGUGUGGGGAAG | |
| 315 | UGAAUUUCUACUGUUGUAGAUAGACAGAUAUUUGCAUUGAGA | |
| 316 | UGAAUUUCUACUGUUGUAGAUGCCAGGGACCGUUUCAGACAG | |
| 317 | UGAAUUUCUACUGUUGUAGAUCGGCUGGCUAGGGAUGAAGAA | |
| 318 | UGAAUUUCUACUGUUGUAGAUAUCACAGGCUCCAGGAAGGGU | |
| 319 | UGAAUUUCUACUGUUGUAGAUUCACAGGCUCCAGGAAGGGUU | |
| 320 | UGAAUUUCUACUGUUGUAGAUGCCUCUGAUUAGGGUGGGGGC | |
| 321 | UGGAUGAAUUUCUACUGUUGUAGAUCCUUGUCAAGGCUAUUGGUCA | |
| 322 | GGGAAUGGAUGAAUUUCUACUGUUGUAGAUCCUUGUCAAGGCUAUUGGUCA | |
| 323 | GUCAAAGGGAAUGGAUGAAUUUCUACUGUUGUAGAUCCUUGUCAAGGCUAUUGGUCA | |
| 324 | AUGUGUUUUUGUCAAAAGACCUUUUUGAAUUUCUACUGUUGUAGAUCCUUGUCAAGGCUAUUGGUCA | |
| 325 | UGGAUAAUUUCUACUCUUGUAGAUCCUUGUCAAGGCUAUUGGUCA | |
| 326 | GGGAAUGGAUAAUUUCUACUCUUGUAGAUCCUUGUCAAGGCUAUUGGUCA | |
| 327 | GUCAAAGGGAAUGGAUAAUUUCUACUCUUGUAGAUCCUUGUCAAGGCUAUUGGUCA | |
| 328 | AUGUGUUUUUGUCAAAAGACCUUUUUAAUUUCUACUCUUGUAGAUCCUUGUCAAGGCUAUUGGUCA | |
| 329 | AUUUCUACUGUUGUAGAUCCUUGUCAAGGCUAUUGGUCA | |
| 330 | UGGAugAAUuuCUacUguuGuagaUcCUUGUCaagGcUAUUGGuca | |
| 331 | GGGAAUGGAUGGAugAAUuuCUacUguuGuagaUcCUUGUCaagGcUAUUGGuca | |
| 332 | GUCAAAGGGAAUGGAugAAUuuCUacUguuGuagaUcCUUGUCaagGcUAUUGGuca | |
| 333 | AUGUGUUUUUGUCAAAAGACCUUUUugAAUuuCUacUguuGuagaUcCUUGUCaagGcUAUUGGuca | |
| 334 | UGGAuuUGAAUuuCUAcUguuGUagaUcCUUGUCaagGcUAUUGGuca | |
| 335 | GGGAAUGGAuuUGAAUuuCUAcUguuGUagaUcCUUGUCaagGcUAUUGGuca | |
| 336 | GUCAAAGGGAAUGGAuuUGAAUuuCUAcUguuGUagaUcCUUGUCaagGcUAUUGGuca | |
| 337 | AUGUGUUUUUGUCAAAAGACCUUUUuuUGAAUuuCUAcUguuGUagaUcCUUGUCaagGcUAUUGGuca | |
| 338 | UGAAUUUCUACUGUUGUAGAUCCUUGUCAAGGCUAUUGGUCACCGAU | |
| 339 | UGAAUUUCUACUGUUGUAGAUCCUUGUCAAGGCUAUUGGUCACGAUA | |
| 340 | UGAAUUUCUACUGUUGUAGAUCCUUGUCAAGGCUAUUGGUGGCUA | |
| 341 | UGAAUUUCUACUGUUGUAGAUCCUUGUCAAGGCUAUUGGUCAUUUUGAUAA | |
| 342 | UGAAUUUCUACUGUUGUAGAUCCUUGUCAAGGCUAUUGGUCGAUA | |
| 343 | UGAAUUUCUACUGUUGUAGACCUUGUCAAGGCUAUUGGUCACUAUA | |
| 344 | UGAAUUUCUACUGUUGUAGACCUUGUCAAGGCUAUUGGUCAUUUUCGAUAG | |
| 345 | AUUUCUACUGUUGUAGACCUUGUCAAGGCUAUUGGUCAUUUUCGAUAG | |
| 346 | UGAAUUUCUACUGUUGUAGACCUUGUCAAGGCUAUUGGUCAUUUUCGAUA | |
| 347 | ugAAUuuCUacUguuGuaGAucCUUGUCaaggcuAUUGGucaSSS | |
| 348 | ugAAUUuCUacUguuGuaGAUcCUUGUCaaggCUAUUGGucaSSSS | |
| 349 | ugAAUUuCUacUguuGUAGAUcCUUGUCaagGCUAUUGGucaSSSSS | |
| 350 | UGAAUUUCUACUGUUGUAGAUCCUUGUCAAGGCUAUUGGUCAg | |
| 351 | UGAAUUUCUACUGUUGUAGAUCCUUGUCAAGGCUAUUGGUCAgg | |
| 352 | UGAAUUUCUACUGUUGUAGAUCCUUGUCAAGGCUAUUGGUCAggg | |
| 353 | gUGAAUUUCUACUGUUGUAGAUCCUUGUCAAGGCUAUUGGUCA | |
| 354 | ggUGAAUUUCUACUGUUGUAGAUCCUUGUCAAGGCUAUUGGUCA | |
| 355 | gggUGAAUUUCUACUGUUGUAGAUCCUUGUCAAGGCUAUUGGUCA | |
| 356 | TGGAUGAAUUUCUACUGUUGUAGAUCCUUGUCAAGGCUAUUGGUCA | |
| 357 | GGGAATGGAUGAAUUUCUACUGUUGUAGAUCCUUGUCAAGGCUAUUGGUCA | |
| 358 | GTCAAAGGGAATGGAUGAAUUUCUACUGUUGUAGAUCCUUGUCAAGGCUAUUGGUCA | |
| 359 | ATGTGTTTTTGTCAAAAGACCTTTTUGAAUUUCUACUGUUGUAGAUCCUUGUCAAGGCUAUUGGUCA | |
| 360 | TGGAUGAAUUUCUACUGUUGUAGAUCCUUGUCAAGGCUAUUGGUCAS | |
| 361 | GGGAATGGAUGAAUUUCUACUGUUGUAGAUCCUUGUCAAGGCUAUUGGUCAS | |
| 362 | GTCAAAGGGAATGGAUGAAUUUCUACUGUUGUAGAUCCUUGUCAAGGCUAUUGGUCAS | |
| 363 | ATGTGTTTTTGTCAAAAGACCTTTTUGAAUUUCUACUGUUGUAGAUCCUUGUCAAGGCUAUUGGUCAS | |
| 364 | UGAAUUUCUACUGUUGUAGAUCCUUGUCAAGGCUAUUGGUCACUAUA | |
| 365 | UGAAUUUCUACUGUUGUAGAUCCUUGUCAAGGCUAUUGGUCAUUUUCGAUAG | |
| 366 | UGAAUUUCUACUGUUGUAGAUCCUUGUCAAGGCUAUUGGUCAUUUUCGAUAGs | |
| 367 | UGAAUUUCUACUGUUGUAGAUCCUUGUCAAGGCUAUUGGUCAUUUUCGAUA | |
| 368 | UGGAugAAUuuCuacUguuGuagaUCAUUGAGAUAGUGUGGGGAAG | |
| 369 | GGGAAUGGAugAAUuuCuacUguuGuagaUCAUUGAGAUAGUGUGGGGAAG | |
| 370 | GUCAAAGGGAAUGGAugAAUuuCuacUguuGuagaUCAUUGAGAUAGUGUGGGGAAG | |
| 371 | AUGUGUUUUUGUCAAAAGACCUUUUugAAUuuCuacUguuGuagaUCAUUGAGAUAGUGUGGGGAAG | |
| 372 | UGGAugAAUuuCuacUguuGuagaUcAUUGAGAuagUgUGGGGaags | |
| 373 | GGGAAUGGAugAAUuuCuacUguuGuagaUcAUUGAGAuagUgUGGGGaagss | |
| 374 | GUCAAAGGGAAUGGAugAAUuuCuacUguuGuagaUcAUUGAGAuagUgUGGGGaagsss | |
| 375 | AUGUGUUUUUGUCAAAAGACCUUUUugAAUuuCuacUguuGuagaUcAUUGAGAuagUgUGGGGaags | |
| 376 | UGGAugAAUuuCuacUguuGuagaUUCACAGGCUCCAGGAAGGGUU | |
| 377 | GGGAAUGGAugAAUuuCuacUguuGuagaUUCACAGGCUCCAGGAAGGGUU | |
| 378 | GUCAAAGGGAAUGGAugAAUuuCuacUguuGuagaUUCACAGGCUCCAGGAAGGGUU | |
| 379 | AUGUGUUUUUGUCAAAAGACCUUUUugAAUuuCuacUguuGuagaUUCACAGGCUCCAGGAAGGGUU | |
| 380 | UGGAugAAUuuCuacUguuGuagaUuCACAGGcuccAgGAAGGguus | |
| 381 | GGGAAUGGAugAAUuuCuacUguuGuagaUuCACAGGcuccAgGAAGGguuss | |
| 382 | GUCAAAGGGAAUGGAugAAUuuCuacUguuGuagaUuCACAGGcuccAgGAAGGguusss | |
| 383 | AUGUGUUUUUGUCAAAAGACCUUUUugAAUuuCuacUguuGuagaUuCACAGGcuccAgGAAGGguussss | |
| 384 | UUUUUUUUugAAUuuCuacUguuGuagaUcAUUGAGAuagUgUGGGGaag | |
| 385 | UGGUGGUGGUGGUUUUUUUUUugAAUuuCuacUguuGuagaUcAUUGAGAuagUgUGGGGaag | |
| 386 | GGGUGGGAGGGCUGGGUUUUUUUUugAAUuuCuacUguuGuagaUcAUUGAGAuagUgUGGGGaag | |
| 387 | GGGUGGGCUGGGAAGGGUUUUUUUUugAAUuuCuacUguuGuagaUcAUUGAGAuagUgUGGGGaag | |
| 388 | GGGAGGGAGGGCUAGGGUUUUUUUUugAAUuuCuacUguuGuagaUcAUUGAGAuagUgUGGGGaag | |
| 389 | GGGCAGGGCUGGGAGGGUUUUUUUUugAAUuuCuacUguuGuagaUcAUUGAGAuagUgUGGGGaag | |
| 390 | GGGCUGGGCUGGGCAGGGUUUUUUUUugAAUuuCuacUguuGuagaUcAUUGAGAuagUgUGGGGaag | |
| 391 | UUGACGCGGUUCUAUCUAGUUACGCGUUAAACCAACUAGAAAUUUUUUUUugAAUuuCuacUguuGuagaUcAUUGAGAuagUgUGGGGaag | |
| 392 | GGAGCAGACGAUAUGGCGUCGCUCCUUUUUUUUugAAUuuCuacUguuGuagaUcAUUGAGAuagUgUGGGGaag | |
| 393 | UUUUUUUUugAAUuuCuacUguuGuagaUcAUUGAGAuagUgUGGGGaagUUUUUUUU | |
| 394 | ugAAUuuCuacUguuGuagaUcAUUGAGAuagUgUGGGGaagUUUUUUUUUGGUGGUGGUGGU | |
| 395 | ugAAUuuCuacUguuGuagaUcAUUGAGAuagUgUGGGGaagUUUUUUUUGGGUGGGAGGGCUGGG | |
| 396 | ugAAUuuCuacUguuGuagaUcAUUGAGAuagUgUGGGGaagUUUUUUUUGGGUGGGCUGGGAAGGG | |
| 397 | ugAAUuuCuacUguuGuagaUcAUUGAGAuagUgUGGGGaagUUUUUUUUGGGAGGGAGGGCUAGGG | |
| 398 | ugAAUuuCuacUguuGuagaUcAUUGAGAuagUgUGGGGaagUUUUUUUUGGGCAGGGCUGGGAGGG | |
| 399 | ugAAUuuCuacUguuGuagaUcAUUGAGAuagUgUGGGGaagUUUUUUUUGGGCUGGGCUGGGCAGGG | |
| 400 | ugAAUuuCuacUguuGuagaUcAUUGAGAuagUgUGGGGaagUUUUUUUUUUGACGCGGUUCUAUCUAGUUACGCGUUAAACCAACUAGAAA | |
| 401 | ugAAUuuCuacUguuGuagaUcAUUGAGAuagUgUGGGGaagUUUUUUUUGGAGCAGACGAUAUGGCGUCGCUCC | |
| 402 | UUUUUUUUugAAUuuCuacUguuGuagaUuCACAGGcuccAgGAAGGguu | |
| 403 | UGGUGGUGGUGGUUUUUUUUUugAAUuuCuacUguuGuagaUuCACAGGcuccAgGAAGGguu | |
| 404 | GGGUGGGAGGGCUGGGUUUUUUUUugAAUuuCuacUguuGuagaUuCACAGGcuccAgGAAGGguu | |
| 405 | GGGUGGGCUGGGAAGGGUUUUUUUUugAAUuuCuacUguuGuagaUuCACAGGcuccAgGAAGGguu | |
| 406 | GGGAGGGAGGGCUAGGGUUUUUUUUugAAUuuCuacUguuGuagaUuCACAGGcuccAgGAAGGguu | |
| 407 | GGGCAGGGCUGGGAGGGUUUUUUUUugAAUuuCuacUguuGuagaUuCACAGGcuccAgGAAGGguu | |
| 408 | GGGCUGGGCUGGGCAGGGUUUUUUUUugAAUuuCuacUguuGuagaUuCACAGGcuccAgGAAGGguu | |
| 409 | UUGACGCGGUUCUAUCUAGUUACGCGUUAAACCAACUAGAAAUUUUUUUUugAAUuuCuacUguuGuagaUuCACAGGcuccAgGAAGGguu | |
| 410 | GGAGCAGACGAUAUGGCGUCGCUCCUUUUUUUUugAAUuuCuacUguuGuagaUuCACAGGcuccAgGAAGGguu | |
| 411 | ugAAUuuCuacUguuGuagaUuCACAGGcuccAgGAAGGguuUUUUUUUU | |
| 412 | ugAAUuuCuacUguuGuagaUuCACAGGcuccAgGAAGGguuUUUUUUUUUGGUGGUGGUGGU | |
| 413 | ugAAUuuCuacUguuGuagaUuCACAGGcuccAgGAAGGguuUUUUUUUUGGGUGGGAGGGCUGGG | |
| 414 | ugAAUuuCuacUguuGuagaUuCACAGGcuccAgGAAGGguuUUUUUUUUGGGUGGGCUGGGAAGGG | |
| 415 | ugAAUuuCuacUguuGuagaUuCACAGGcuccAgGAAGGguuUUUUUUUUGGGAGGGAGGGCUAGGG | |
| 416 | ugAAUuuCuacUguuGuagaUuCACAGGcuccAgGAAGGguuUUUUUUUUGGGCAGGGCUGGGAGGG | |
| 417 | ugAAUuuCuacUguuGuagaUuCACAGGcuccAgGAAGGguuUUUUUUUUGGGCUGGGCUGGGCAGGG | |
| 418 | ugAAUuuCuacUguuGuagaUuCACAGGcuccAgGAAGGguuUUUUUUUUUUGACGCGGUUCUAUCUAGUUACGCGUUAAACCAACUAGAAA | |
| 419 | ugAAUuuCuacUguuGuagaUuCACAGGcuccAgGAAGGguuUUUUUUUUGGAGCAGACGAUAUGGCGUCGCUCC | |
| 420 | UAAUUUCUACUCUUGUAGAUCCUUGUCAAGGCUAUUGGUCAUGACCAAUAGCCU | |
| 421 | UAAUUUCUACUCUUGUAGAUCCUUGUCAAGGCUAUUGGUCAUGACCAAUAGCC | |
| 422 | UGAAUUUCUACUGUUGUAGAUCCUUGUCAAGGCUAUUGGUCAUGACCAAUAGCCUU | |
| 423 | AAUUUCUACUGUUGUAGAUCCUUGUCAAGGCUAUUGGUCAUGACCAAUAGCC | |
| 424 | UAAUUUCUACUCUUGUAGAUCCUUGUCAAGGCUAUUGGUCAUGACCAAUAGCCUS | |
| 425 | UAAUUUCUACUCUUGUAGAUCCUUGUCAAGGCUAUUGGUCAUGACCAAUAGCCS | |
| 426 | UGAAUUUCUACUGUUGUAGAUCCUUGUCAAGGCUAUUGGUCAUGACCAAUAGCCUUs | |
| 427 | AAUUUCUACUGUUGUAGAUCCUUGUCAAGGCUAUUGGUCAUGACCAAUAGCCs | |
| 428 | acaUgaggaUcacccaUgUUUUUUUUUUGAAUUUCUACUGUUGUAGAUcAUUGAGAuagUgUGGGGaag | |
| 429 | UGAAUUUCUACUGUUGUAGAUcAUUGAGAuagUgUGGGGaagUUUUUUUUacaUgaggaUcacccaUgU | |
| 430 | acaUgaggaUcacccaUgUUUUUUUUUUGAAUUUCUACUGUUGUAGAUuCACAGGcuccAgGAAGGguu | |
| 431 | UGAAUUUCUACUGUUGUAGAUuCACAGGcuccAgGAAGGguuUUUUUUUUacaUgagga UcacccaUgU | |
| 432 | UGAAUUUCUACUGUUGUAGAUUUCCGAUUCAGUCAUUCCAGU | |
| 433 | UGAAUUUCUACUGUUGUAGAUCCUUGUUCCGAUUCAGUCAUU | |
| 434 | UGAAUUUCUACUGUUGUAGAUGCCUUGUUCCGAUUCAGUCAU | |
| 435 | UGAAUUUCUACUGUUGUAGAUUAGCCUUUGCCUUGUUCCGAU | |
| 436 | UGAAUUUCUACUGUUGUAGAUAUAGCCUUUGCCUUGUUCCGA | |
| 437 | UGAAUUUCUACUGUUGUAGAUUAUAGCCUUUGCCUUGUUCCG | |
| 438 | UGAAUUUCUACUGUUGUAGAUUUAUAGCCUUUGCCUUGUUCC | |
| 439 | UGAAUUUCUACUGUUGUAGAUUUUAUAGCCUUUGCCUUGUUC | |
| 440 | UGAAUUUCUACUGUUGUAGAUUUUUAUAGCCUUUGCCUUGUU | |
| 441 | UGAAUUUCUACUGUUGUAGAUUUUUUAUAGCCUUUGCCUUGU | |
| 442 | UGAAUUUCUACUGUUGUAGAUAGAUAGUGUGGGGAAGGGGCC | |
| 443 | UGAAUUUCUACUGUUGUAGAUGCAGUAUCCUCUUGGGGGCCC | |
| 444 | UGAAUUUCUACUGUUGUAGAUGCAUUGAGAUAGUGUGGGGAA | |
| 445 | UGAAUUUCUACUGUUGUAGAUGGGGCCCCUUCCCCACACUAU | |
| 446 | UGAAUUUCUACUGUUGUAGAUCAGACAGAUAUUUGCAUUGAG | |
| 447 | UGAAUUUCUACUGUUGUAGAUCCCACACUAUCUCAAUGCAAA | |
| 448 | UGAAUUUCUACUGUUGUAGAUAGCCAGGGACCGUUUCAGACA | |
| 449 | UGAAUUUCUACUGUUGUAGAUGUCAAGGCAAGGCUGGCCAAC | |
| 450 | UGAAUUUCUACUGUUGUAGAUUCAAGGCUAUUGGUCAAGGCA | |
| 451 | UGAAUUUCUACUGUUGUAGAUGCCUUGUCAAGGCUAUUGGUC | |
| 452 | UGAAUUUCUACUGUUGUAGAUGCCAGCCUUGCCUUGACCAAU | |
| 453 | UGAAUUUCUACUGUUGUAGAUGUCAAGUUUGCCUUGUCAAGG | |
| 454 | UGAAUUUCUACUGUUGUAGAUCCUUGACCAAUAGCCUUGACA | |
| 455 | UGAAUUUCUACUGUUGUAGAUACCAAUAGCCUUGACAAGGCA | |
| 456 | UGAAUUUCUACUGUUGUAGAUACAAGGCAAACUUGACCAAUA | |
| 457 | UGAAUUUCUACUGUUGUAGAUACCAAUAGUCUUAGAGUAUCC | |
| 458 | UGAAUUUCUACUGUUGUAGAUGAGUAUCCAGUGAGGCCAGGG | |
| 459 | UGAAUUUCUACUGUUGUAGAUAUCCCUAGCCAGCCGCCGGCC | |
| 460 | UGAAUUUCUACUGUUGUAGAUUUCAUCCCUAGCCAGCCGCCG | |
| 461 | UGAAUUUCUACUGUUGUAGAUUUCUUCAUCCCUAGCCAGCCG | |
| 462 | UGAAUUUCUACUGUUGUAGAUAUUCUUCAUCCCUAGCCAGCC | |
| 463 | UGAAUUUCUACUGUUGUAGAUUAUUCUUCAUCCCUAGCCAGC | |
| 464 | UGAAUUUCUACUGUUGUAGAUCUUUUAUUCUUCAUCCCUAGC | |
| 465 | UGAAUUUCUACUGUUGUAGAUCAGAAGCGAGUGUGUGGAACU | |
| 466 | UGAAUUUCUACUGUUGUAGAUAGCAGUUCCACACACUCGCUU | |
| 467 | UGAAUUUCUACUGUUGUAGAUAUAACCUCAGACGUUCCAGAA | |
| 468 | UGAAUUUCUACUGUUGUAGAUUUGAUAACCUCAGACGUUCCA | |
| 469 | UGAAUUUCUACUGUUGUAGAUCACACACUCGCUUCUGGAACG | |
| 470 | UGAAUUUCUACUGUUGUAGAUUGGAACGUCUGAGGUUAUCAA | |
| 471 | UGAAUUUCUACUGUUGUAGAUUCAAUAAGCUCCUAGUCCAGA | |
| 472 | UGAAUUUCUACUGUUGUAGAUCACUGGAAUCAGCUAUCUGCU | |
| 473 | UGAAUUUCUACUGUUGUAGAUGCACUGGAAUCAGCUAUCUGC | |
| 474 | UGAAUUUCUACUGUUGUAGAUCCUGUAUGGACUUUGCACUGG | |
| 475 | UGAAUUUCUACUGUUGUAGAUUUACCUGUAUGGACUUUGCAC | |
| 476 | UGAAUUUCUACUGUUGUAGAUUGGCCUAUGUUAUUACCUGUA | |
| 477 | UGAAUUUCUACUGUUGUAGAUCUGGCCUAUGUUAUUACCUGU | |
| 478 | UGAAUUUCUACUGUUGUAGAUUCUGGCCUAUGUUAUUACCUG | |
| 479 | UGAAUUUCUACUGUUGUAGAUCAGUGCAAAGUCCAUACAGGU | |
| 480 | UGAAUUUCUACUGUUGUAGAUUGUCUAAGAGUAGAUGCCAUA | |
| 481 | UGAAUUUCUACUGUUGUAGAUACCCUGGUGUGUUAUGUCUAA | |
| 482 | UGAAUUUCUACUGUUGUAGAUUAUUGACCCUGGUGUGUUAUG | |
| 483 | UGAAUUUCUACUGUUGUAGAUAAAGUUGUAUUGACCCUGGUG | |
| 484 | UGAAUUUCUACUGUUGUAGAUGACAUAACACACCAGGGUCAA | |
| 485 | UGAAUUUCUACUGUUGUAGAUCACUAGACUAGCUUCAAAGUU | |
| 486 | UGAAUUUCUACUGUUGUAGAUGCUUGCACUAGACUAGCUUCA | |
| 487 | UGAAUUUCUACUGUUGUAGAUGAAGCUAGUCUAGUGCAAGCU | |
| 488 | UGAAUUUCUACUGUUGUAGAUAAGCUAGUCUAGUGCAAGCUA | |
| 489 | UGAAUUUCUACUGUUGUAGAUCUGGAGCCUGUGAUAAAAGCA | |
| 490 | UGAAUUUCUACUGUUGUAGAUCUUUUAUCACAGGCUCCAGGA | |
| 491 | UGAAUUUCUACUGUUGUAGAUUAUCACAGGCUCCAGGAAGGG | |
| 492 | UGAAUUUCUACUGUUGUAGAUUACCCCACCCACGCCCCCACC | |
| 493 | UGAAUUUCUACUGUUGUAGAUGGCCUCUGAUUAGGGUGGGGG | |
| 494 | UGAAUUUCUACUGUUGUAGAUGGGUGGGGGCGUGGGUGGGGU | |
| 495 | UGAAUUUCUACUGUUGUAGAUCGAAAGAGGCCCCCCUGGGCA | |
| 496 | UGAAUUUCUACUGUUGUAGAUGCCCAGGGGGGCCUCUUUCGG | |
| 497 | UGAAUUUCUACUGUUGUAGAUCCCAGGGGGGCCUCUUUCGGA | |
| 498 | UGAAUUUCUACUGUUGUAGAUUCCAUCACCAAGAGAGCCUUC | |
| 499 | UGAAUUUCUACUGUUGUAGAUCGGAAGGCUCUCUUGGUGAUG | |
| 500 | UGAAUUUCUACUGUUGUAGAUGGAAGGCUCUCUUGGUGAUGG | |
| 501 | UGAAUUUCUACUGUUGUAGAUAGAAAUAAAAUCCAAUUCUCC | |
| 502 | UGAAUUUCUACUGUUGUAGAUCCAUUGAGAAAUAAAAUCCAA | |
| 503 | UGAAUUUCUACUGUUGUAGAUAUUCCCAUUGAGAAAUAAAAU | |
| 504 | UGAAUUUCUACUGUUGUAGAUCAUUCCCAUUGAGAAAUAAAA | |
| 505 | UGAAUUUCUACUGUUGUAGAUGUGAUGGAGAAUUGGAUUUUA | |
| 506 | UGAAUUUCUACUGUUGUAGAUUUUCAUUCCCAUUGAGAAAUA | |
| 507 | UGAAUUUCUACUGUUGUAGAUGAUUUUAUUUCUCAAUGGGAA | |
| 508 | UGAAUUUCUACUGUUGUAGAUUAUUUCUCAAUGGGAAUGAAA | |
| 509 | UGAAUUUCUACUGUUGUAGAUAUUUCUCAAUGGGAAUGAAAU | |
| 510 | UGAAUUUCUACUGUUGUAGAUUUUCUCAAUGGGAAUGAAAUA | |
| 511 | UGAAUUUCUACUGUUGUAGAUCUCAAUGGGAAUGAAAUAAUU | |
| 512 | UGAAUUUCUACUGUUGUAGAUUCAAUGGGAAUGAAAUAAUUU | |
| 513 | UGAAUUUCUACUGUUGUAGAUGGAGUCCACACGGCAUGGCAU | |
| 514 | UGAAUUUCUACUGUUGUAGAUGGGAGUCCACACGGCAUGGCA | |
| 515 | UGAAUUUCUACUGUUGUAGAUUGGGAGUCCACACGGCAUGGC | |
| 516 | UGAAUUUCUACUGUUGUAGAUCAAUUUUGGGAGUCCACACGG | |
| 517 | UGAAUUUCUACUGUUGUAGAUACAAUUUUGGGAGUCCACACG | |
| 518 | UGAAUUUCUACUGUUGUAGAUGUAUGCCAUGCCGUGUGGACU | |
| 519 | UGAAUUUCUACUGUUGUAGAUUAUGCCAUGCCGUGUGGACUC | |
| 520 | UGAAUUUCUACUGUUGUAGAUACCUCCUUUACAAUUUUGGGA | |
| 521 | UGAAUUUCUACUGUUGUAGAUUAAAGGAGGUGAAGCUUCCCC | |
| 522 | UGAAUUUCUACUGUUGUAGAUCCUUGUCAAGGCUAUUGGUCASS | |
| 523 | UGAAUUUCUACUGUUGUAGAUCCUUGUCAAGGCUAUUGGUCAUUUU | |
| 524 | UGAAUUUCUACUGUUGUAGAUCCUUGUCAAGGCUAUUGGUCAUUUUU | |
| 525 | UGAAUUUCUACUGUUGUAGAUCCUUGUCAAGGCUAUUGGUCAUUUUUU | |
| 526 | UGAAUUUCUACUGUUGUAGAUCCUUGUCAAGGCUAUUGGUCAUUUUUUU | |
| 527 | UGAAUUUCUACUGUUGUAGAUCCUUGUCAAGGCUAUUGGUCAUUUUUUUU | |
| 528 | UGAAUUUCUACUGUUGUAGAUCCUUGUCAAGGCUAUUGGUCAUUUUUUUUU | |
| 529 | UGAAUGAAGGCUAUUGGUCAUUUUS | |
| 530 | UGAAUGAAGGCUAUUGGUCAUUUUUs | |
| 531 | UGAAUGAAGGCUAUUGGUCAUUUUUUs | |
| 532 | UGAAUGAAGGCUAUUGGUCAUUUUUUUs | |
| 533 | UGAAUGAAGGCUAUUGGUCAUUUUUUUUs | |
| 534 | UGAAUGAAGGCUAUUGGUCAUUUUUUUUUs | |
| 535 | UGAAUUUCUACUGUUGUAGAUCUGGUACCAGGAUGAUGCAAU | |
| 536 | UGAAUUUCUACUGUUGUAGAUCAUCAUCCUGGUACCAGGAAG | |
| 537 | UGAAUUUCUACUGUUGUAGAUCCUUCCUGGUACCAGGAUGAU | |
| 538 | GATAAACAATCGTCATCCTC | |
| 539 | GGATACCAACCCGCGGGGTC | |
| 540 | TGTTTATCAACGTCATCTAG | |
| 541 | ATCTACTTAGAAAGCGAACA | |
| 542 | TTGCTTTTATCACAGGCTCC | |
| 543 | CTAACAGTTGCTTTTATCAC | |
| 544 | GTCTCGGTGGTGGACTAAAC | |
| 545 | CTGAAGGGATACCAACCCGC | |
| 546 | TGAACCAGACCACGGCCCGT | |
| 547 | ATCACCCGAGTGCCTTTGAC | |
| 548 | CTGGAGCCTGTGATAAAAGC | |
| 549 | CCTTGTCAAGGCTATTGGTC | |
| 550 | CATTGAGATAGTGTGGGGAA | |
| 551 | CTTGTCAAGGCTATTGGTCA | |
| 552 | ATTGAGATAGTGTGGGGAAG | |
| 553 | CTTGACCAATAGCCTTGACA | |
| 554 | GTGGGGAAGGGGCCCCCAAG | |
| 555 | CAAGGCTATTGGTCAAGGCA | |
| 556 | CTTGTCAAGGCTATTGGTC | |
| 557 | TAGCCTTGACAAGGCAAACT | |
| 558 | TATCTGTCTGAAACGGTCCC | |
| 559 | GTGGGGAAGGGGCCCCCAA | |
| 560 | AGATATTTGCATTGAGATAG | |
| 561 | GGCAAGGCTGGCCAACCCAT | |
| 562 | AGGCAAGGCTGGCCAACCCA | |
| 563 | CCTTGTCAAGGCTATTGGTCA | |
| 564 | CCTTGTCAAGGCTATTGGTCAA | |
| 565 | CCTTGTCAAGGCTATTGGTCAAG | |
| 566 | CATTGAGATAGTGTGGGGAAGGG | |
| 567 | CCCAGGGGGGCCTCTTTCGG | |
| 568 | TCACAGGCTCCAGGAAGGGT | |
| 569 | AAGCTAGTCTAGTGCAAGCT | |
| 570 | CACTGGAATCAGCTATCTGC | |
| 571 | AGCCATCTCACTACAGATAA | |
| 572 | CCCATGGGGCACAGTCAGGC | |
| 573 | TTTTATCACAGGCTCCAGGA | |
| 574 | TTTATCACAGGCTCCAGGAA | |
| 575 | ACTGAATCGGAACAAGGCAA | |
| 576 | CTAACAGTTGCTTTTATCAC | |
| 577 | ATCACAGGCTCCAGGAAGGGT | |
| 578 | TCACAGGCTCCAGGAAGGGTT | |
| 579 | GCCTCTGATTAGGGTGGGGGC | |
| 580 | CATTGAGATAGTGTGGGGAAG | |
| 581 | AGACAGATATTTGCATTGAGA | |
| 582 | GCCAGGGACCGTTTCAGACAG | |
| 583 | CGGCTGGCTAGGGATGAAGAA | |
| 584 | ccttgtcaaggctattggt | |
| 585 | CCUUGUCAAGGCUAUUGGUCA | |
| 586 | UCCUUGUCAAGGCUAUUGGUCA | |
| 587 | TTCCGATTCAGTCATTCCAGT | |
| 588 | CCTTGTTCCGATTCAGTCATT | |
| 589 | GCCTTGTTCCGATTCAGTCAT | |
| 590 | TAGCCTTTGCCTTGTTCCGAT | |
| 591 | ATAGCCTTTGCCTTGTTCCGA | |
| 592 | TATAGCCTTTGCCTTGTTCCG | |
| 593 | TTATAGCCTTTGCCTTGTTCC | |
| 594 | TTTATAGCCTTTGCCTTGTTC | |
| 595 | TTTTATAGCCTTTGCCTTGTT | |
| 596 | TTTTTATAGCCTTTGCCTTGT | |
| 597 | AGATAGTGTGGGGAAGGGGCC | |
| 598 | GCAGTATCCTCTTGGGGGCCC | |
| 599 | GCATTGAGATAGTGTGGGGAA | |
| 600 | GGGGCCCCTTCCCCACACTAT | |
| 601 | CAGACAGATATTTGCATTGAG | |
| 602 | CCCACACTATCTCAATGCAAA | |
| 603 | AGCCAGGGACCGTTTCAGACA | |
| 604 | GTCAAGGCAAGGCTGGCCAAC | |
| 605 | TCAAGGCTATTGGTCAAGGCA | |
| 606 | GCCTTGTCAAGGCTATTGGTC | |
| 607 | GCCAGCCTTGCCTTGACCAAT | |
| 608 | GTCAAGTTTGCCTTGTCAAGG | |
| 609 | CCTTGACCAATAGCCTTGACA | |
| 610 | ACCAATAGCCTTGACAAGGCA | |
| 611 | ACAAGGCAAACTTGACCAATA | |
| 612 | ACCAATAGTCTTAGAGTATCC | |
| 613 | GAGTATCCAGTGAGGCCAGGG | |
| 614 | ATCCCTAGCCAGCCGCCGGCC | |
| 615 | TTCATCCCTAGCCAGCCGCCG | |
| 616 | TTCTTCATCCCTAGCCAGCCG | |
| 617 | ATTCTTCATCCCTAGCCAGCC | |
| 618 | TATTCTTCATCCCTAGCCAGC | |
| 619 | CTTTTATTCTTCATCCCTAGC | |
| 620 | CAGAAGCGAGTGTGTGGAACT | |
| 621 | AGCAGTTCCACACACTCGCTT | |
| 622 | ATAACCTCAGACGTTCCAGAA | |
| 623 | TTGATAACCTCAGACGTTCCA | |
| 624 | CACACACTCGCTTCTGGAACG | |
| 625 | TGGAACGTCTGAGGTTATCAA | |
| 626 | TCAATAAGCTCCTAGTCCAGA | |
| 627 | CACTGGAATCAGCTATCTGCT | |
| 628 | GCACTGGAATCAGCTATCTGC | |
| 629 | CCTGTATGGACTTTGCACTGG | |
| 630 | TTACCTGTATGGACTTTGCAC | |
| 631 | TGGCCTATGTTATTACCTGTA | |
| 632 | CTGGCCTATGTTATTACCTGT | |
| 633 | TCTGGCCTATGTTATTACCTG | |
| 634 | CAGTGCAAAGTCCATACAGGT | |
| 635 | TGTCTAAGAGTAGATGCCATA | |
| 636 | ACCCTGGTGTGTTATGTCTAA | |
| 637 | TATTGACCCTGGTGTGTTATG | |
| 638 | AAAGTTGTATTGACCCTGGTG | |
| 639 | GACATAACACACCAGGGTCAA | |
| 640 | CACTAGACTAGCTTCAAAGTT | |
| 641 | GCTTGCACTAGACTAGCTTCA | |
| 642 | GAAGCTAGTCTAGTGCAAGCT | |
| 643 | AAGCTAGTCTAGTGCAAGCTA | |
| 644 | CTGGAGCCTGTGATAAAAGCA | |
| 645 | CTTTTATCACAGGCTCCAGGA | |
| 646 | TATCACAGGCTCCAGGAAGGG | |
| 647 | TACCCCACCCACGCCCCCACC | |
| 648 | GGCCTCTGATTAGGGTGGGGG | |
| 649 | GGGTGGGGGCGTGGGTGGGGT | |
| 650 | CGAAAGAGGCCCCCCTGGGCA | |
| 651 | GCCCAGGGGGGCCTCTTTCGG | |
| 652 | CCCAGGGGGGCCTCTTTCGGA | |
| 653 | TCCATCACCAAGAGAGCCTTC | |
| 654 | CGGAAGGCTCTCTTGGTGATG | |
| 655 | GGAAGGCTCTCTTGGTGATGG | |
| 656 | AGAAATAAAATCCAATTCTCC | |
| 657 | CCATTGAGAAATAAAATCCAA | |
| 658 | ATTCCCATTGAGAAATAAAAT | |
| 659 | CATTCCCATTGAGAAATAAAA | |
| 660 | GTGATGGAGAATTGGATTTTA | |
| 661 | TTTCATTCCCATTGAGAAATA | |
| 662 | GATTTTATTTCTCAATGGGAA | |
| 663 | TATTTCTCAATGGGAATGAAA | |
| 664 | ATTTCTCAATGGGAATGAAAT | |
| 665 | TTTCTCAATGGGAATGAAATA | |
| 666 | CTCAATGGGAATGAAATAATT | |
| 667 | TCAATGGGAATGAAATAATTT | |
| 668 | GGAGTCCACACGGCATGGCAT | |
| 669 | GGGAGTCCACACGGCATGGCA | |
| 670 | TGGGAGTCCACACGGCATGGC | |
| 671 | CAATTTTGGGAGTCCACACGG | |
| 672 | ACAATTTTGGGAGTCCACACG | |
| 673 | GTATGCCATGCCGTGTGGACT | |
| 674 | TATGCCATGCCGTGTGGACTC | |
| 675 | ACCTCCTTTACAATTTTGGGA | |
| 676 | TAAAGGAGGTGAAGCTTCCCC | |
| 677 | CTGGTACCAGGATGATGCAAT | |
| 678 | CATCATCCTGGTACCAGGAAG | |
| 679 | CCTTCCTGGTACCAGGATGAT | |
| 680 | u*g*AAUuuCUacUguuGuagaUcCUUGUCaagGcUAUUGGU*c*a | |
| 681 | u*g*AAUuuCUacUguuGuaGAucCUUGUCaaggcuAUUGGU*c*a | |
| 682 | u*g*AAUUuCUacUguuGuaGAUcCUUGUCaaggCUAUUGGU*c*a | |
| 683 | u*g*AAUUuCUacUguuGUAGAUcCUUGUCaagGCUAUUGGU*c*a | |
| 684 | u*g*AAUuuCUacUguuGuagaUcCUUGUCaagGcUAUUGGu*c*a | |
| 685 | UGGAUGAAUUUCUACUGUUGUAGAUAGUGGGGGUGAAUUCAGUGU | |
| 686 | GGGAAUGGAUGAAUUUCUACUGUUGUAGAUAGUGGGGGUGAAUUCAGUGU | |
| 687 | GUCAAAGGGAAUGGAUGAAUUUCUACUGUUGUAGAUAGUGGGGGUGAAUUCAGUGU | |
| 688 | AUGUGUUUUUGUCAAAAGACCUUUUUGAAUUUCUACUGUUGUAGAUAGUGGGGGUGAAUUCAGUGU | |
| 689 | UGGAUGAAUUUCUACUGUUGUAGAUCCUUGUCAAGGCUAUUGGUCA | |
| 690 | GGGAAUGGAUGAAUUUCUACUGUUGUAGAUCCUUGUCAAGGCUAUUGGUCA | |
| 691 | GUCAAAGGGAAUGGAUGAAUUUCUACUGUUGUAGAUCCUUGUCAAGGCUAUUGGUCA | |
| 692 | AUGUGUUUUUGUCAAAAGACCUUUUUGAAUUUCUACUGUUGUAGAUCCUUGUCAAGGCUAUUGGUCA | |
| 693 | T*G*GAugAAUuuCUacUguuGuagaUcCUUGUCaagGcUAUUGGu*c*a | |
| 694 | G*G*GAATGGAugAAUuuCUacUguuGuagaUcCUUGUCaagGUAUUGGu*c*a | |
| 695 | G*T*CAAAGGGAATGGAugAAUuuCUacUguuGuagaUcCUUGUCaagGcUAUUGGu*c*a | |
| 696 | A*T*GTGTTTTTGTCAAAAGACCTTTTugAAUuuCUacUguuGuagaUcCUUGUCaagGcUAUUGGu*c*a | |
| 697 | T*G*GAUGAAUUUCUACUGUUGUAGAUCCUUGUCAAGGCUAUUGGU*c*a | |
| 698 | G*G*GAATGGAUGAAUUUCUACUGUUGUAGAUCCUUGUCAAGGCUAUUGGU*c*a | |
| 699 | G*T*CAAAGGGAATGGAUGAAUUUCUACUGUUGUAGAUCCUUGUCAAGGCUAUUGGU*c*a | |
| 700 | A*T*GTGTTTTTGTCAAAAGACCTTTTUGAAUUUCUACUGUUGUAGAUCCUUGUCAAGGCUAUUGGU*c*a | |
| 701 | u*g*GAugAAUuuCUacUguuGuagaUcCUUGUCaagGcUAUUGGu*c*a | |
| 702 | g*g*GAAUGGAugAAUuuCUacUguuGuagaUcCUUGUCaagGcUAUUGGu*c*a | |
| 703 | g* u*CAAAGGGAAUGGAugAAUuuCUacUguuGuagaUcCUUGUCaagGUAUUGGu*c*a | |
| 704 | a*u*GUGUUUUUGUCAAAAGACCUUUUugAAUuuCUacUguuGuagaUcCUUGUCaagGUAUUGGu*c*a | |
| 705 | u*g*GAuuUGAAUuuCUAcUguuGUagaUcCUUGUCaagGcUAUUGGu*c*a | |
| 706 | g*g*GAAUGGAuuUGAAUuuCUAcUguuGUagaUcCUUGUCaagGcUAUUGGu*c*a | |
| 707 | g*u*CAAAGGGAAUGGAuuUGAAUuuCUAcUguuGUagaUcCUUGUCaagGcUAUUGGu*c*a | |
| 708 | a*u*GUGUUUUUGUCAAAAGACCUUUUuuUGAAUuuCUAcUguuGUagaUcCUUGUCaagGUAUUGGu*c*a | |
| 709 | u*g*GAugAAUuuCUacUguuGuagaUaGUGGGGgugAaUUCAGU*g*u | |
| 710 | g*g*GAAUGGAugAAUuuCUacUguuGuagaUaGUGGGGgugAaUUCAGU*g*u | |
| 711 | g*u*CAAAGGGAAUGGAugAAUuuCUacUguuGuagaUaGUGGGGgugAaUUCAGU*g*u | |
| 712 | a*u*GUGUUUUUGUCAAAAGACCUUUUugAAUuuCUacUguuGuagaUaGUGGGGgugAaUUCAGU*g*u | |
| 713 | u*g*AAUUUCUACUGUUGUAGAUCCUUGUCAAGGCUAUUGGUCACCG*a*u | |
| 714 | u*g*AAUUUCUACUGUUGUAGAUCCUUGUCAAGGCUAUUGGUCACGA*u*a | |
| 715 | u*g*AAUUUCUACUGUUGUAGAUCCUUGUCAAGGCUAUUGGUGGC*u*a | |
| 716 | u*g*AAUUUCUACUGUUGUAGAUCCUUGUCAAGGCUAUUGGUCAUUUUGAU*a*a | |
| 717 | u*g*AAUUUCUACUGUUGUAGAUCCUUGUCAAGGCUAUUGGUCGA*u*a | |
| 718 | u*g*AAUuuCUacUguuGuaGAucCUUGUCaaggcuAUUGGu*c*a | |
| 719 | u*g*AAUUuCUacUguuGuaGAUcCUUGUCaaggCUAUUGGu*c*a | |
| 720 | u*g*AAUUuCUacUguuGUAGAUcCUUGUCaagGCUAUUGGu*c*a | |
| 721 | UGGAGUGUGACAAUGGUGUUUG | |
| 722 | UGUAGUGUUUCCUACUUUAUGGA | |
| 723 | UGUCAGUUUGUCAAAUACCCCA | |
| 724 | UUAAUGCUAAUCGUGAUAGGGGUU | |
| 725 | UCUCCCAACCCUUGUACCAGUG | |
| 726 | ACAGUAGUCUGCACAUUGGUUA | |
| 727 | UCUCACACAGAAAUCGCACCCGU | |
| 728 | AACAUUCAACGCUGUCGGUGAGU | |
| 729 | UGAGGUAGGAGGUUGUAUAGUU | |
| 730 | UAAUACUGCCUGGUAAUGAUGA | |
| 731 | UAAUACUGCCGGGUAAUGAUGGA | |
| 732 | GUGAAAUGUUUAGGACCACUAG | |
| 733 | UCCUUCAUUCCACCGGAGUCUG | |
| 734 | CAUUAUUACUUUUGGUACGCG | |
| 735 | UCGUACCGUGAGUAAUAAUGCG | |
| 736 | CAAACACCATTGTCACACTCCA | |
| 737 | TCCATAAAGTAGGAAACACTACA | |
| 738 | TGGGGTATTTGACAAACTGACA | |
| 739 | AACCCCTATCACGATTAGCATTAA | |
| 740 | CACTGGTACAAGGGTTGGGAGA | |
| 741 | TAACCAATGTGCAGACTACTGT | |
| 742 | ACGGGTGCGATTTCTGTGTGAGA | |
| 743 | ACTCACCGACAGCGTTGAATGTT | |
| 744 | AACTATACAACCTCCTACCTCA | |
| 745 | TCATCATTACCAGGCAGTATTA | |
| 746 | TCCATCATTACCCGGCAGTATTA | |
| 747 | CTAGTGGTCCTAAACATTTCAC | |
| 748 | CAGACTCCGGTGGAATGAAGGA | |
| 749 | CGCGTACCAAAAGTAATAATG | |
| 750 | CGCATTATTACTCACGGTACGA | |
| 751 | ACACAAAUACCAGUCCAGCGGUUUUAGAGCUAGAAAUAGCAAGUUAAAAUAAGGCUAGUCCGUUAUCAACUUCAAAAAGUGGCA | |
| | CCGAGUCGGUGCUUUU | |
| 752 | NNNNNNNNNNNNNNNNNNNNGUUUUAGAGCUAGAAAUAGCAAGUUAAAAUAAGGCUAGUCCGUUAUCAACUUCAAAAAGUGG | |
| | CACCGAGUCGGUGCUUUU | |
| 753 | AGCCACGAAAA | |
| 754 | MARIIDEFCGQMNGYTRSITLRNRLVPIGKTEENLKQFLEKDLERATAYPDIKNLIDAIHRNVIEDTLSKVALNWNEIFNILATYQNEKD | Q565K |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENKESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 755 | MARIIDEFCGQMNGYTRSITLRNRLVPIGKTEENLKQFLEKDLERATAYPDIKNLIDAIHRNVIEDTLSKVALNWNEIFNILATYQNEKD | N588I |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPINKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 756 | MARIIDEFCGQMNGYTRSITLRNRLVPIGKTEENLKQFLEKDLERATAYPDIKNLIDAIHRNVIEDTLSKVALNWNEIFNILATYQNEKD | I584S |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGSYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPL | |
| | SEIDKMIDEGKLFLFQIYNKDFAKKATGKKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 757 | MARIIDEFCGQMNGYTRSITLRNRLVPIGKTEENLKQFLEKDLERATAYPDIKNLIDAIHRNVIEDTLSKVALNWNEIFNILATYQNEKD | V453E |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KESILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 758 | MARIIDEFCGQMNGYTRSITLRNRLVPIGKTEENLKQFLEKDLERATAYPDIKNLIDAIHRNVIEDTLSKVALNWNEIFNILATYQNEKD | Q475H |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLHQEKNAAIPIKAYLDEVQNLYHHLKLVDYRGEEQKDSTFYSKHDEILTDLSQIVPLYNKVRNFVTKK | |
| | LGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPL | |
| | SEIDKMIDEGKLFLFQIYNKDFAKKATGKKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 759 | MARIIDEFCGQMNGYTRSITLRNRLVPIGKTEENLKQFLEKDLERATAYPDIKNLIDAIHRNVIEDTLSKVALNWNEIFNILATYQNEKD | N400S |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKISKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 760 | MARIIDEFCGQMNGYTRSITLRNRLVPIGKTEENLKQFLEKDLERATAYPDIKNLIDAIHRNVIEDTLSKVALNWNEIFNILATYQNEKD | I471N |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHNQDLQQEKNAAIPIKAYLDEVQNLYHHLKLVDYRGEEQKDSTFYSKHDEILTDLSQIVPLYNKVRNFVTKK | |
| | LGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPL | |
| | SEIDKMIDEGKLFLFQIYNKDFAKKATGKKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 761 | MARIIDEFCGQMNGYTRSITLRNRLVPIGKTEENLKQFLEKDLERATAYPDIKNLIDAIHRNVIEDTLSKVALNWNEIFNILATYQNEKD | E544D |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GDSKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 762 | MARIIDEFCGQMNGYTRSITLRNRLVPIGKTEENLKQFLEKDLERATAYPDIKNLIDAIHRNVIEDTLSKVALNWNEIFNILATYQNEKD | H496N |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYNHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 763 | MARIIDEFCGQMNGYTRSITLRNRLVPIGKTEENLKQFLEKDLERATAYPDIKNLIDAIHRNVIEDTLSKVALNWNEIFNILATYQNEKD | V534F |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKFRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRINKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 764 | MARIIDEFCGQMNGYTRSITLRNRLVPIGKTEENLKQFLEKDLERATAYPDIKNLIDAIHRNVIEDTLSKVALNWNEIFNILATYQNEKD | D577G |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKGGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRINKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 765 | MARIIDEFCGQMNGYTRSITLRNRLVPIGKTEENLKQFLEKDLERATAYPDIKNLIDAIHRNVIEDTLSKVALNWNEIFNILATYQNEKD | K401M |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINMEIAKKDCSFTELQLAYERYCTEHNLPVEKFCKDYFDIVDYRGNGAKSEK | |
| | TKVSILSEILETFLQLDFDHIQDLQQEKNAAIPIKAYLDEVQNLYHHLKLVDYRGEEQKDSTFYSKHDEILTDLSQIVPLYNKVRNFVTKK | |
| | LGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPL | |
| | SEIDKMIDEGKLFLFQIYNKDFAKKATGKKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 766 | MARIIDEFCGQMNGYTRSITLRNRLVPIGKTEENLKQFLEKDLERATAYPDIKNLIDAIHRNVIEDTLSKVALNWNEIFNILATYQNEKD | K549E |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIELNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 767 | MARIIDEFCGQMNGYTRSITLRNRLVPIGKTEENLKQFLEKDLERATAYPDIKNLIDAIHRNVIEDTLSKVALNWNEIFNILATYQNEKD | K579I |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGIYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 768 | MARIIDEFCGQMNGYTRSITLRNRLVPIGKTEENLKQFLEKDLERATAYPDIKNLIDAIHRNVIEDTLSKVALNWNEIFNILATYQNEKD | K509E |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQEDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 769 | MARIIDEFCGQMNGYTRSITLRNRLVPIGKTEENLKQFLEKDLERATAYPDIKNLIDAIHRNVIEDTLSKVALNWNEIFNILATYQNEKD | K595T |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFATKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 770 | MARIIDEFCGQMNGYTRSITLRNRLVPIGKTEENLKQFLEKDLERATAYPDIKNLIDAIHRNVIEDTLSKVALNWNEIFNILATYQNEKD | V501D |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLDDYRGEEQKDSTFYSKHDEILTDLSQIVPLYNKVRNFVTKK | |
| | LGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPL | |
| | SEIDKMIDEGKLFLFQIYNKDFAKKATGKKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 771 | MARIIDEFCGQMNGYTRSITLRNRLVPIGKTEENLKQFLEKDLERATAYPDIKNLIDAIHRNVIEDTLSKVALNWNEIFNILATYQNEKD | S568I |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESINDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 772 | MARIIDEFCGQMNGYTRSITLRNRLVPIGKTEENLKQFLEKDLERATAYPDIKNLIDAIHRNVIEDTLSKVALNWNEIFNILATYQNEKD | H470L |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDLIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 773 | MARIIDEFCGQMNGYTRSITLRNRLVPIGKTEENLKQFLEKDLERATAYPDIKNLIDAIHRNVIEDTLSKVALNWNEIFNILATYQNEKD | G443R |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRRNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 774 | MARIIDEFCGQMNGYTRSITLRNRLVPIGKTEENLKQFLEKDLERATAYPDIKNLIDAIHRNVIEDTLSKVALNWNEIFNILATYQNEKD | P591S |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKSKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 775 | MARIIDEFCGQMNGYTRSITLRNRLVPIGKTEENLKQFLEKDLERATAYPDIKNLIDAIHRNVIEDTLSKVALNWNEIFNILATYQNEKD | K516T |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSTHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 776 | MARIIDEFCGQMNGYTRSITLRNRLVPIGKTEENLKQFLEKDLERATAYPDIKNLIDAIHRNVIEDTLSKVALNWNEIFNILATYQNEKD | E383V |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLVKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 777 | MARIIDEFCGQMNGYTRSITLRNRLVPIGKTEENLKQFLEKDLERATAYPDIKNLIDAIHRNVIEDTLSKVALNWNEIFNILATYQNEKD | L474Q |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDQQQEKNAAIPIKAYLDEVQNLYHHLKLVDYRGEEQKDSTFYSKHDEILTDLSQIVPLYNKVRNFVTKK | |
| | LGESKKIKLNFDCPTLANGWDENQESSNDAIILRKDGKYYLGIYNPNNKPKFAKKDSIVGDCYEKMAYKQIALPMGLGAFVRKCFGT | |
| | AQKYGWGCPENCLNSEGKIIIKDEEAKGNLEAIIDCYKDFLNKYEKDGFKYKDYNFSFLDSASYEKLSDFFNDVKPQGYKLSFTSIPL | |
| | SEIDKMIDEGKLFLFQIYNKDFAKKATGKKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 778 | MARIIDEFCGQMNGYTRSITLRNRLVPIGKTEENLKQFLEKDLERATAYPDIKNLIDAIHRNVIEDTLSKVALNWNEIFNILATYQNEKD | H470N |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDNIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 779 | MARIIDEFCGQMNGYTRSITLRNRLVPIGKTEENLKQFLEKDLERATAYPDIKNLIDAIHRNVIEDTLSKVALNWNEIFNILATYQNEKD | E402V |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKVIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 780 | MARIIDEFCGQMNGYTRSITLRNRLVPIGKTEENLKQFLEKDLERATAYPDIKNLIDAIHRNVIEDTLSKVALNWNEIFNILATYQNEKD | Q465R |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLRLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 781 | MARIIDEFCGQMNGYTRSITLRNRLVPIGKTEENLKQFLEKDLERATAYPDIKNLIDAIHRNVIEDTLSKVALNWNEIFNILATYQNEKD | F537I |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNIVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 782 | MARIIDEFCGQMNGYTRSITLRNRLVPIGKTEENLKQFLEKDLERATAYPDIKNLIDAIHRNVIEDTLSKVALNWNEIFNILATYQNEKD | K745M |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGMKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 783 | MARIIDEFCGQMNGYTRSITLRNRLVPIGKTEENLKQFLEKDLERATAYPDIKNLIDAIHRNVIEDTLSKVALNWNEIFNILATYQNEKD | T838S |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGSVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 784 | MARIIDEFCGQMNGYTRSITLRNRLVPIGKTEENLKQFLEKDLERATAYPDIKNLIDAIHRNVIEDTLSKVALNWNEIFNILATYQNEKD | N735I |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYIKDFAKKATGKKNLHTLYWENLFSVENLQDVVLKLNGEAELFWREASIKKDKVIVHKKGSILVNRTTTDGKSI | |
| | PEAIYQEIYQLKNKMADSISDEAKRLLESGTVVCKVATHDIVKDKHFTENTYLFHCPITMNFKAKDRTNKEFNNHVLEVLNKNPDIKVI | |
| | GLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 785 | MARIIDEFCGQMNGYTRSITLRNRLVPIGKTEENLKQFLEKDLERATAYPDIKNLIDAIHRNVIEDTLSKVALNWNEIFNILATYQNEKD | I723V |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMVDEGKLFLFQIYNKDFAKKATGKKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 786 | MARIIDEFCGQMNGYTRSITLRNRLVPIGKTEENLKQFLEKDLERATAYPDIKNLIDAIHRNVIEDTLSKVALNWNEIFNILATYQNEKD | K821I |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNIMADSISDEAKRLLESGTVVCKVATHDIVKDKHFTENTYLFHCPITMNFKAKDRTNKEFNNHVLEVLNKNPDIKVI | |
| | GLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 787 | MARIIDEFCGQMNGYTRSITLRNRLVPIGKTEENLKQFLEKDLERATAYPDIKNLIDAIHRNVIEDTLSKVALNWNEIFNILATYQNEKD | K721M |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDMMIDEGKLFLFQIYNKDFAKKATGKKNLHTLYWENLFSVENLQDVVLKLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 788 | MARIIDEFCGQMNGYTRSITLRNRLVPIGKTEENLKQFLEKDLERATAYPDIKNLIDAIHRNVIEDTLSKVALNWNEIFNILATYQNEKD | A810V |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREASIKKDKVIVHKKGSILVNRTTTDGKS | |
| | IPEVIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 789 | MARIIDEFCGQMNGYTRSITLRNRLVPIGKTEENLKQFLEKDLERATAYPDIKNLIDAIHRNVIEDTLSKVALNWNEIFNILATYQNEKD | K768M |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLMLNGEAELFWREASIKKDKVIVHKKGSILVNRTTTDGK | |
| | SIPEAIYQEIYQLKNKMADSISDEAKRLLESGTVVCKVATHDIVKDKHFTENTYLFHCPITMNFKAKDRTNKEFNNHVLEVLNKNPDIK | |
| | VIGLDRGERHLLYLSLINQKGEIECQKTLNLVEQVRNDKTVSVNYHEKLVHKEGSRDAARKNWQTIGNIKELKEGYLSAVVHEIASLM | |
| | VKHNAIVVMEDLNFGFKRGRFAVERQIYQKFENMLIEKLNYLVFKDRKVTEPGGVLNAYQLANKSAKVTDVYKQCGWLFYIPAAYTS | |
| | KIDPRTGFANLFITKGLTNVEKKKEFFGKFDSIRYDATESCFVFSFDYAKICDNADYKKKWDVYTRGTRLVYNKTERKNVSVNPTEELQ | |
| | CVFDEFGIKWNTGEDLIESISLIPAEKSNAKFFDVLLRMFNATLQMRNSVPNTDTDYLVSPVKAEDGSFFDSREEFKKGGDARLPIDC | |
| | DANGAYHIALKGLYLLLNDFNRDNKGVIQNISNKDWFKFVQEKVYKDX | |
| 790 | MARIIDEFCGQMNGYTRSITLRNRLVPIGKTEENLKQFLEKDLERATAYPDIKNLIDAIHRNVIEDTLSKVALNWNEIFNILATYQNEKD | S781R |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFLFQIYNKDFAKKATGKKNLHTLYWENLFSVENLQDVVLKLNGEAELFWREARIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 791 | MARIIDEFCGQMNGYTRSITLRNRLVPIGKTEENLKQFLEKDLERATAYPDIKNLIDAIHRNVIEDTLSKVALNWNEIFNILATYQNEKD | E725D |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDDGKLFLFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRINKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 792 | MARIIDEFCGQMNGYTRSITLRNRLVPIGKTEENLKQFLEKDLERATAYPDIKNLIDAIHRNVIEDTLSKVALNWNEIFNILATYQNEKD | L730S |
| | KKKKAAIKKDLEKLQSGARKKIVEAFKKNPDFEKLFKEGLFKELLPELIKSAPVDEIAVKTKALECFNRFSTYFTGFHDNRKNMYSEEAK | |
| | STAISYRIVNENFPKFFANIKLFNYLKEHFPRIIIDTEESLKDYLKGKKLDSVFSIDGFNSVLAQSGIDFYNTVIGGISGEAGTKKTQGLNE | |
| | KINLARQQLSKEEKNKLRGKMVVLFKQILSDRETSSFIPVGFANKEEVYSTVKEFNNSIAEKAVSKVRDLFLHREEFTLNEIFVPAKSLT | |
| | DFSQAIFGSWSILSEGLFLLEKDSMKKALSESQEEKINKEIAKKDCSFTELQLAYERYCTEHNLPVEKFCKDYFDIVDYRGNGAKSEKT | |
| | KVSILSEILETFLQLDFDHIQDLQQEKNAAIPIKAYLDEVQNLYHHLKLVDYRGEEQKDSTFYSKHDEILTDLSQIVPLYNKVRNFVTKKL | |
| | GESKKIKLNFDCPTLANGWDENQESSNDAIILRKDGKYYLGIYNPNNKPKFAKKDSIVGDCYEKMAYKQIALPMGLGAFVRKCFGTA | |
| | QKYGWGCPENCLNSEGKIIIKDEEAKGNLEAIIDCYKDFLNKYEKDGFKYKDYNFSFLDSASYEKLSDFFNDVKPQGYKLSFTSIPLS | |
| | EIDKMIDEGKLFSFQIYNKDFAKKATGKKNLHTLYWENLFSVENLQDVVLKLNGEAELFWREASIKKDKVIVHKKGSILVNRTTTDGKS | |
| | IPEAIYQEIYQLKNKMADSISDEAKRLLESGTVVCKVATHDIVKDKHFTENTYLFHCPITMNFKAKDRTNKEFNNHVLEVLNKNPDIKV | |
| | IGLDRGERHLLYLSLINQKGEIECQKTLNLVEQVRNDKTVSVNYHEKLVHKEGSRDAARKNWQTIGNIKELKEGYLSAVVHEIASLMV | |
| | KHNAIVVMEDLNFGFKRGRFAVERQIYQKFENMLIEKLNYLVFKDRKVTEPGGVLNAYQLANKSAKVTDVYKQCGWLFYIPAAYTSKI | |
| | DPRTGFANLFITKGLTNVEKKKEFFGKFDSIRYDATESCFVFSFDYAKICDNADYKKKWDVYTRGTRLVYNKTERKNVSVNPTEELQC | |
| | VFDEFGIKWNTGEDLIESISLIPAEKSNAKFFDVLLRMFNATLQMRNSVPNTDTDYLVSPVKAEDGSFFDSREEFKKGGDARLPIDCD | |
| | ANGAYHIALKGLYLLLNDFNRDNKGVIQNISNKDWFKFVQEKVYKDX | |
| 793 | ATGGCTAGGATAATTGATGAATTTTGTGGACAGATGAATGGATACAGCCGTTCCATCACGCTGCGTAACCGCCTGGTGCCGATCG | Q565K |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATAAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 794 | ATGGCTAGGATAATTGATGAATTTTGTGGACAGATGAATGGATACAGCCGTTCCATCACGCTGCGTAACCGCCTGGTGCCGATCG | N588I |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGATCAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 795 | ATGGCTAGGATAATTGATGAATTTTGTGGACAGATGAATGGATACAGCCGTTCCATCACGCTGCGTAACCGCCTGGTGCCGATCG | I584S |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCAGTTACAACCCGAACAACAAAC | |
| | CGAAATTCGCGAAGAAGGACAGCATCGTGGGTGATTGCTATGAAAAAATGGCATACAAGCAAATCGCACTGCCGATGGGTTTGG | |
| | GTGCATTTGTGCGCAAATGCTTTGGCACCGCTCAGAAATACGGTTGGGGTTGCCCGGAGAATTGCCTGAACAGCGAGGGTAAA | |
| | ATTATCATCAAAGACGAGGAGGCGAAGGGTAATCTGGAGGCAATCATCGACTGTTACAAAGATTTTCTGAACAAGTACGAAAAGG | |
| | ACGGCTTCAAGTACAAGGACTACAATTTTTCCTTCCTGGACTCTGCCAGCTATGAAAAACTGTCTGATTTCTTCAACGACGTTAAA | |
| | CCGCAGGGCTATAAGCTGAGCTTTACGTCCATCCCACTGTCAGAGATCGATAAGATGATTGATGAAGGTAAACTGTTTTTGTTTCA | |
| | GATATATAACAAAGACTTCGCGAAGAAGGCGACGGGGAAGAAGAACCTGCATACCCTTTATTGGGAAAACCTATTCTCAGTCGA | |
| | GAATCTGCAAGATGTGGTGTTGAAGCTCAATGGCGAAGCCGAGCTGTTCTGGCGTGAGGCTAGCATCAAAAAGGACAAGGTGA | |
| | TCGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 796 | ATGGCTAGGATAATTGATGAATTTTGTGGACAGATGAATGGATACAGCCGTTCCATCACGCTGCGTAACCGCCTGGTGCCGATCG | V453E |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGAGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 797 | ATGGCTAGGATAATTGATGAATTTTGTGGACAGATGAATGGATACAGCCGTTCCATCACGCTGCGTAACCGCCTGGTGCCGATCG | Q475H |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCATCAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 798 | ATGGCTAGGATAATTGATGAATTTTGTGGACAGATGAATGGATACAGCCGTTCCATCACGCTGCGTAACCGCCTGGTGCCGA | N400S |
| | TCGGCAAGACCGAGGAAAATCTGAAACAGTTTCTGGAGAAGGACTTAGAGCGCGCAACTGCGTACCCGGACATCAAAAAC | |
| | CTGATTGACGCTATCCATCGTAATGTCATTGAGGACACCTTAAGCAAAGTGGCCCTGAACTGGAACGAAATCTTTAATATCCT | |
| | CGCGACGTACCAGAACGAAAAAGATAAAAAGAAAAAGGCGGCGATCAAAAAGGACCTGGAGAAGCTCCAAAGCGGTGCA | |
| | CGTAAAAAGATTGTTGAGGCATTCAAAAAGAACCCGGATTTTGAAAAACTGTTCAAAGAGGGTCTGTTTAAAGAGTTGCTGC | |
| | CTGAACTGATCAAAAGCGCACCGGTTGATGAAATCGCGGTTAAAACCAAGGCGCTGGAATGCTTCAACCGTTTTAGCACCT | |
| | ATTTCACGGGTTTCCACGACAATCGCAAAAACATGTACTCGGAAGAAGCTAAATCTACCGCAATTAGCTATCGCATCGTGAA | |
| | CGAAAATTTCCCGAAATTTTTTGCCAATATCAAGTTGTTCAACTATCTGAAAGAGCACTTTCCGCGAATCATCATTGATACCGA | |
| | AGAGTCCCTGAAAGACTATTTGAAGGGCAAAAAGCTGGATTCTGTATTTAGCATTGACGGCTTTAACAGCGTGCTGGCGCA | |
| | GAGCGGGATTGATTTCTATAACACCGTGATAGGCGGTATTTCAGGTGAGGCCGGCACCAAAAAAACGCAGGGTCTTAACGA | |
| | GAAGATTAACTTAGCGCGCCAGCAATTATCCAAGGAGGAGAAGAACAAGCTGCGCGGTAAAATGGTGGTGCTGTTTAAACA | |
| | GATCTTGTCTGATCGTGAAACCAGCAGCTTTATCCCAGTGGGTTTTGCCAACAAGGAGGAGGTGTACTCTACGGTGAAAGA | |
| | ATTCAACAACTCGATTGCCGAAAAGGCGGTGTCCAAGGTGCGTGATCTATTCCTGCACCGCGAAGAATTCACGCTAAACGA | |
| | GATTTTCGTCCCGGCAAAGAGCTTGACCGACTTCAGTCAGGCCATCTTCGGATCCTGGTCTATTTTGAGTGAGGGCCTGTT | |
| | CCTGCTGGAGAAGGACAGCATGAAAAAGGCTCTAAGCGAATCCCAAGAAGAAAAGATCAGTAAGGAGATTGCGAAAAAAG | |
| | ACTGCTCCTTTACCGAACTACAACTGGCGTACGAGCGCTACTGCACCGAGCACAACTTGCCGGTTGAAAAATTCTGCAAAG | |
| | ACTACTTCGACATCGTCGATTACAGGGGAAACGGTGCGAAAAGCGAAAAAACAAAAGTGAGCATCCTGAGCGAAATCCTG | |
| | GAAACCTTTTTACAATTGGACTTTGACCACATTCAGGACCTGCAACAAGAAAAGAACGCTGCGATTCCTATCAAAGCATATCT | |
| | TGACGAAGTTCAGAATTTGTACCACCACCTGAAGTTGGTCGATTATCGTGGCGAAGAGCAGAAAGACTCAACCTTTTATAGC | |
| | AAACACGATGAAATCCTGACCGATCTTTCTCAGATCGTCCCATTGTATAATAAAGTCCGTAACTTTGTTACCAAAAAACTGGG | |
| | TGAGAGCAAAAAGATCAAGCTGAACTTCGATTGCCCGACTCTGGCTAATGGCTGGGATGAGAATCAAGAGTCCAGCAACG | |
| | ATGCAATTATCCTGAGAAAGGACGGCAAATACTATCTGGGCATTTACAACCCGAACAACAAACCGAAATTCGCGAAGAAGGA | |
| | CAGCATCGTGGGTGATTGCTATGAAAAAATGGCATACAAGCAAATCGCACTGCCGATGGGTTTGGGTGCATTTGTGCGCAA | |
| | ATGCTTTGGCACCGCTCAGAAATACGGTTGGGGTTGCCCGGAGAATTGCCTGAACAGCGAGGGTAAAATTATCATCAAAGA | |
| | CGAGGAGGCGAAGGGTAATCTGGAGGCAATCATCGACTGTTACAAAGATTTTCTGAACAAGTACGAAAAGGACGGCTTCAA | |
| | GTACAAGGACTACAATTTTTCCTTCCTGGACTCTGCCAGCTATGAAAAACTGTCTGATTTCTTCAACGACGTTAAACCGCAG | |
| | GGCTATAAGCTGAGCTTTACGTCCATCCCACTGTCAGAGATCGATAAGATGATTGATGAAGGTAAACTGTTTTTGTTTCAGAT | |
| | ATATAACAAAGACTTCGCGAAGAAGGCGACGGGGAAGAAGAACCTGCATACCCTTTATTGGGAAAACCTATTCTCAGTCGA | |
| | GAATCTGCAAGATGTGGTGTTGAAGCTCAATGGCGAAGCCGAGCTGTTCTGGCGTGAGGCTAGCATCAAAAAGGACAAGG | |
| | TGATCGTTCACAAAAAAGGTTCTATTCTGGTGAATCGTACCACCACGGATGGTAAGAGCATCCCGGAGGCCATTTATCAAGA | |
| | AATCTATCAATTAAAAAACAAAATGGGGGATTCCATCAGCGACGAAGCGAAACGCCTTCTAGAGTCCGGCACCGTTGTCTGT | |
| | AAAGTCGCCACTCATGATATCGTTAAGGATAAACATTTTACTGAAAACACGTATCTGTTCCATTGTCCGATTACCATGAACTTC | |
| | AAGGCAAAAGACCGTACCAATAAGGAATTTAACAATCATGTTCTGGAGGTTCTTAACAAGAATCCGGATATTAAGGTGATCGG | |
| | CTTAGACCGTGGCGAACGTCATCTGCTGTACCTGAGCCTGATCAATCAGAAGGGTGAAATTGAGTGCCAGAAAACGCTGAA | |
| | CCTAGTTGAGCAAGTTCGTAATGATAAGACCGTTTCCGTTAATTATCATGAAAAACTGGTGCACAAAGAAGGCAGCCGTGAT | |
| | GCTGCGAGAAAAAACTGGCAGACCATTGGCAATATTAAAGAGCTGAAGGAGGGCTACCTGTCGGCGGTGGTGCACGAGAT | |
| | TGCGAGTCTGATGGTAAAGCACAATGCGATCGTTGTAATGGAAGACCTGAATTTCGGCTTTAAGCGCGGTCGTTTTGCTGT | |
| | GGAGCGTCAGATCTACCAAAAGTTCGAAAACATGCTGATTGAGAAGCTGAACTATCTGGTTTTTAAAGACCGTAAAGTGACG | |
| | GAGCCGGGTGGCGTTCTGAACGCGTACCAGTTGGCGAACAAGAGCGCGAAGGTGACAGATGTTTATAAACAATGCGGTTG | |
| | GCTGTTCTATATCCCGGCAGCGTACACCAGCAAAATTGATCCGCGTACCGGTTTCGCGAATCTGTTCATTACCAAGGGTCTG | |
| | ACCAATGTTGAGAAGAAGAAGGAGTTTTTCGGCAAGTTCGACTCGATTCGTTATGATGCGACCGAGTCTTGTTTCGTTTTTT | |
| | CCTTCGACTACGCAAAGATTTGTGACAATGCTGATTACAAGAAGAAATGGGATGTTTACACCCGTGGTACTCGTCTCGTTTA | |
| | CAACAAGACCGAACGCAAGAACGTGAGCGTCAACCCAACCGAAGAGTTGCAGTGTGTTTTTGATGAGTTCGGCATCAAGT | |
| | GGAATACCGGCGAAGACCTGATTGAATCTATTAGCCTGATCCCGGCGGAAAAGTCCAACGCGAAGTTCTTCGACGTTTTGT | |
| | TGCGTATGTTTAACGCGACTCTTCAGATGAGAAACAGCGTGCCGAATACCGACACCGACTACTTGGTCAGCCCGGTGAAAG | |
| | CTGAAGACGGCAGCTTTTTCGACAGCCGCGAGGAGTTTAAGAAAGGTGGCGATGCCCGCCTGCCGATAGACTGCGATGC | |
| | TAATGGTGCGTACCATATTGCCCTGAAAGGCTTGTACTTGTTGTTGAATGATTTCAACCGTGATAACAAGGGCGTCATCCAAA | |
| | ACATCTCTAACAAGGACTGGTTTAAATTCGTGCAGGAGAAAGTTTACAAAGACTAA | |
| 799 | ATGGCTAGGATAATTGATGAATTTTGTGGACAGATGAATGGATACAGCCGTTCCATCACGCTGCGTAACCGCCTGGTGCCGATCG | I471N |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACAATCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 800 | ATGGCTAGGATAATTGATGAATTTTGTGGACAGATGAATGGATACAGCCGTTCCATCACGCTGCGTAACCGCCTGGTGCCGATCG | E544D |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGATAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 801 | ATGGCTAGGATAATTGATGAATTTTGTGGACAGATGAATGGATACAGCCGTTCCATCACGCTGCGTAACCGCCTGGTGCCGATCG | H496N |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACAACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 802 | ATGGCTAGGATAATTGATGAATTTTGTGGACAGATGAATGGATACAGCCGTTCCATCACGCTGCGTAACCGCCTGGTGCCGA | V534F |
| | TCGGCAAGACCGAGGAAAATCTGAAACAGTTTCTGGAGAAGGACTTAGAGCGCGCAACTGCGTACCCGGACATCAAAAAC | |
| | CTGATTGACGCTATCCATCGTAATGTCATTGAGGACACCTTAAGCAAAGTGGCCCTGAACTGGAACGAAATCTTTAATATCCT | |
| | CGCGACGTACCAGAACGAAAAAGATAAAAAGAAAAAGGCGGCGATCAAAAAGGACCTGGAGAAGCTCCAAAGCGGTGCA | |
| | CGTAAAAAGATTGTTGAGGCATTCAAAAAGAACCCGGATTTTGAAAAACTGTTCAAAGAGGGTCTGTTTAAAGAGTTGCTGC | |
| | CTGAACTGATCAAAAGCGCACCGGTTGATGAAATCGCGGTTAAAACCAAGGCGCTGGAATGCTTCAACCGTTTTAGCACCT | |
| | ATTTCACGGGTTTCCACGACAATCGCAAAAACATGTACTCGGAAGAAGCTAAATCTACCGCAATTAGCTATCGCATCGTGAA | |
| | CGAAAATTTCCCGAAATTTTTTGCCAATATCAAGTTGTTCAACTATCTGAAAGAGCACTTTCCGCGAATCATCATTGATACCGA | |
| | AGAGTCCCTGAAAGACTATTTGAAGGGCAAAAAGCTGGATTCTGTATTTAGCATTGACGGCTTTAACAGCGTGCTGGCGCA | |
| | GAGCGGGATTGATTTCTATAACACCGTGATAGGCGGTATTTCAGGTGAGGCCGGCACCAAAAAAACGCAGGGTCTTAACGA | |
| | GAAGATTAACTTAGCGCGCCAGCAATTATCCAAGGAGGAGAAGAACAAGCTGCGCGGTAAAATGGTGGTGCTGTTTAAACA | |
| | GATCTTGTCTGATCGTGAAACCAGCAGCTTTATCCCAGTGGGTTTTGCCAACAAGGAGGAGGTGTACTCTACGGTGAAAGA | |
| | ATTCAACAACTCGATTGCCGAAAAGGCGGTGTCCAAGGTGCGTGATCTATTCCTGCACCGCGAAGAATTCACGCTAAACGA | |
| | GATTTTCGTCCCGGCAAAGAGCTTGACCGACTTCAGTCAGGCCATCTTCGGATCCTGGTCTATTTTGAGTGAGGGCCTGTT | |
| | CCTGCTGGAGAAGGACAGCATGAAAAAGGCTCTAAGCGAATCCCAAGAAGAAAAGATCAATAAGGAGATTGCGAAAAAAGA | |
| | CTGCTCCTTTACCGAACTACAACTGGCGTACGAGCGCTACTGCACCGAGCACAACTTGCCGGTTGAAAAATTCTGCAAAGA | |
| | CTACTTCGACATCGTCGATTACAGGGGAAACGGTGCGAAAAGCGAAAAAACAAAAGTGAGCATCCTGAGCGAAATCCTGG | |
| | AAACCTTTTTACAATTGGACTTTGACCACATTCAGGACCTGCAACAAGAAAAGAACGCTGCGATTCCTATCAAAGCATATCTT | |
| | GACGAAGTTCAGAATTTGTACCACCACCTGAAGTTGGTCGATTATCGTGGCGAAGAGCAGAAAGACTCAACCTTTTATAGCA | |
| | AACACGATGAAATCCTGACCGATCTTTCTCAGATCGTCCCATTGTATAATAAATTCCGTAACTTTGTTACCAAAAAACTGGGT | |
| | GAGAGCAAAAAGATCAAGCTGAACTTCGATTGCCCGACTCTGGCTAATGGCTGGGATGAGAATCAAGAGTCCAGCAACGAT | |
| | GCAATTATCCTGAGAAAGGACGGCAAATACTATCTGGGCATTTACAACCCGAACAACAAACCGAAATTCGCGAAGAAGGAC | |
| | AGCATCGTGGGTGATTGCTATGAAAAAATGGCATACAAGCAAATCGCACTGCCGATGGGTTTGGGTGCATTTGTGCGCAAA | |
| | TGCTTTGGCACCGCTCAGAAATACGGTTGGGGTTGCCCGGAGAATTGCCTGAACAGCGAGGGTAAAATTATCATCAAAGAC | |
| | GAGGAGGCGAAGGGTAATCTGGAGGCAATCATCGACTGTTACAAAGATTTTCTGAACAAGTACGAAAAGGACGGCTTCAAG | |
| | TACAAGGACTACAATTTTTCCTTCCTGGACTCTGCCAGCTATGAAAAACTGTCTGATTTCTTCAACGACGTTAAACCGCAGG | |
| | GCTATAAGCTGAGCTTTACGTCCATCCCACTGTCAGAGATCGATAAGATGATTGATGAAGGTAAACTGTTTTTGTTTCAGATA | |
| | TATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGT | |
| | GATCGTTCACAAAAAAGGTTCTATTCTGGTGAATCGTACCACCACGGATGGTAAGAGCATCCCGGAGGCCATTTATCAAGAA | |
| | ATCTATCAATTAAAAAACAAAATGGCGGATTCCATCAGCGACGAAGCGAAACGCCTTCTAGAGTCCGGCACCGTTGTCTGTA | |
| | AAGTCGCCACTCATGATATCGTTAAGGATAAACATTTTACTGAAAACACGTATCTGTTCCATTGTCCGATTACCATGAACTTCA | |
| | AGGCAAAAGACCGTACCAATAAGGAATTTAACAATCATGTTCTGGAGGTTCTTAACAAGAATCCGGATATTAAGGTGATCGG | |
| | CTTAGACCGTGGCGAACGTCATCTGCTGTACCTGAGCCTGATCAATCAGAAGGGTGAAATTGAGTGCCAGAAAACGCTGAA | |
| | CCTAGTTGAGCAAGTTCGTAATGATAAGACCGTTTCCGTTAATTATCATGAAAAACTGGTGCACAAAGAAGGCAGCCGTGAT | |
| | GCTGCGAGAAAAAACTGGCAGACCATTGGCAATATTAAAGAGCTGAAGGAGGGCTACCTGTCGGCGGTGGTGCACGAGAT | |
| | TGCGAGTCTGATGGTAAAGCACAATGCGATCGTTGTAATGGAAGACCTGAATTTCGGCTTTAAGCGCGGTCGTTTTGCTGT | |
| | GGAGCGTCAGATCTACCAAAAGTTCGAAAACATGCTGATTGAGAAGCTGAACTATCTGGTTTTTAAAGACCGTAAAGTGACG | |
| | GAGCCGGGTGGCGTTCTGAACGCGTACCAGTTGGCGAACAAGAGCGCGAAGGTGACAGATGTTTATAAACAATGCGGTTG | |
| | GCTGTTCTATATCCCGGCAGCGTACACCAGCAAAATTGATCCGCGTACCGGTTTCGCGAATCTGTTCATTACCAAGGGTCTG | |
| | ACCAATGTTGAGAAGAAGAAGGAGTTTTTCGGCAAGTTCGACTCGATTCGTTATGATGCGACCGAGTCTTGTTTCGTTTTTT | |
| | CCTTCGACTACGCAAAGATTTGTGACAATGCTGATTACAAGAAGAAATGGGATGTTTACACCCGTGGTACTCGTCTCGTTTA | |
| | CAACAAGACCGAACGCAAGAACGTGAGCGTCAACCCAACCGAAGAGTTGCAGTGTGTTTTTGATGAGTTCGGCATCAAGT | |
| | GGAATACCGGCGAAGACCTGATTGAATCTATTAGCCTGATCCCGGCGGAAAAGTCCAACGCGAAGTTCTTCGACGTTTTGT | |
| | TGCGTATGTTTAACGCGACTCTTCAGATGAGAAACAGCGTGCCGAATACCGACACCGACTACTTGGTCAGCCCGGTGAAAG | |
| | CTGAAGACGGCAGCTTTTTCGACAGCCGCGAGGAGTTTAAGAAAGGTGGCGATGCCCGCCTGCCGATAGACTGCGATGC | |
| | TAATGGTGCGTACCATATTGCCCTGAAAGGCTTGTACTTGTTGTTGAATGATTTCAACCGTGATAACAAGGGCGTCATCCAAA | |
| | ACATCTCTAACAAGGACTGGTTTAAATTCGTGCAGGAGAAAGTTTACAAAGACTAA | |
| 803 | ATGGCTAGGATAATTGATGAATTTTGTGGACAGATGAATGGATACAGCCGTTCCATCACGCTGCGTAACCGCCTGGTGCCGATCG | D577G |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGGCGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 804 | ATGGCTAGGATAATTGATGAATTTTGTGGACAGATGAATGGATACAGCCGTTCCATCACGCTGCGTAACCGCCTGGTGCCGATCG | K401M |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATATGGAGATTGCGAAAAAAGACTGCTCCTTTACCGAACTACAACTGGCGTACGAGCGCTACTGCA | |
| | CCGAGCACAACTTGCCGGTTGAAAAATTCTGCAAAGACTACTTCGACATCGTCGATTACAGGGGAAACGGTGCGAAAAGCGAA | |
| | AAAACAAAAGTGAGCATCCTGAGCGAAATCCTGGAAACCTTTTTACAATTGGACTTTGACCACATTCAGGACCTGCAACAAGAAA | |
| | AGAACGCTGCGATTCCTATCAAAGCATATCTTGACGAAGTTCAGAATTTGTACCACCACCTGAAGTTGGTCGATTATCGTGGCGAA | |
| | GAGCAGAAAGACTCAACCTTTTATAGCAAACACGATGAAATCCTGACCGATCTTTCTCAGATCGTCCCATTGTATAATAAAGTCCG | |
| | TAACTTTGTTACCAAAAAACTGGGTGAGAGCAAAAAGATCAAGCTGAACTTCGATTGCCCGACTCTGGCTAATGGCTGGGATGAG | |
| | AATCAAGAGTCCAGCAACGATGCAATTATCCTGAGAAAGGACGGCAAATACTATCTGGGCATTTACAACCCGAACAACAAACCG | |
| | AAATTCGCGAAGAAGGACAGCATCGTGGGTGATTGCTATGAAAAAATGGCATACAAGCAAATCGCACTGCCGATGGGTTTGGGT | |
| | GCATTTGTGCGCAAATGCTTTGGCACCGCTCAGAAATACGGTTGGGGTTGCCCGGAGAATTGCCTGAACAGCGAGGGTAAAAT | |
| | TATCATCAAAGACGAGGAGGCGAAGGGTAATCTGGAGGCAATCATCGACTGTTACAAAGATTTTCTGAACAAGTACGAAAAGGAC | |
| | GGCTTCAAGTACAAGGACTACAATTTTTCCTTCCTGGACTCTGCCAGCTATGAAAAACTGTCTGATTTCTTCAACGACGTTAAACC | |
| | GCAGGGCTATAAGCTGAGCTTTACGTCCATCCCACTGTCAGAGATCGATAAGATGATTGATGAAGGTAAACTGTTTTTGTTTCAGAT | |
| | ATATAACAAAGACTTCGCGAAGAAGGCGACGGGGAAGAAGAACCTGCATACCCTTTATTGGGAAAACCTATTCTCAGTCGAGAA | |
| | TCTGCAAGATGTGGTGTTGAAGCTCAATGGCGAAGCCGAGCTGTTCTGGCGTGAGGCTAGCATCAAAAAGGACAAGGTGATCG | |
| | TTCACAAAAAAGGTTCTATTCTGGTGAATCGTACCACCACGGATGGTAAGAGCATCCCGGAGGCCATTTATCAAGAAATCTATCAA | |
| | TTAAAAAACAAAATGGCGGATTCCATCAGCGACGAAGCGAAACGCCTTCTAGAGTCCGGCACCGTTGTCTGTAAAGTCGCCAC | |
| | TCATGATATCGTTAAGGATAAACATTTTACTGAAAACACGTATCTGTTCCATTGTCCGATTACCATGAACTTCAAGGCAAAAGACCGT | |
| | ACCAATAAGGAATTTAACAATCATGTTCTGGAGGTTCTTAACAAGAATCCGGATATTAAGGTGATCGGCTTAGACCGTGGCGAACG | |
| | TCATCTGCTGTACCTGAGCCTGATCAATCAGAAGGGTGAAATTGAGTGCCAGAAAACGCTGAACCTAGTTGAGCAAGTTCGTAAT | |
| | GATAAGACCGTTTCCGTTAATTATCATGAAAAACTGGTGCACAAAGAAGGCAGCCGTGATGCTGCGAGAAAAAACTGGCAGACC | |
| | ATTGGCAATATTAAAGAGCTGAAGGAGGGCTACCTGTCGGCGGTGGTGCACGAGATTGCGAGTCTGATGGTAAAGCACAATGC | |
| | GATCGTTGTAATGGAAGACCTGAATTTCGGCTTTAAGCGCGGTCGTTTTGCTGTGGAGCGTCAGATCTACCAAAAGTTCGAAAAC | |
| | ATGCTGATTGAGAAGCTGAACTATCTGGTTTTTAAAGACCGTAAAGTGACGGAGCCGGGTGGCGTTCTGAACGCGTACCAGTTG | |
| | GCGAACAAGAGCGCGAAGGTGACAGATGTTTATAAACAATGCGGTTGGCTGTTCTATATCCCGGCAGCGTACACCAGCAAAATT | |
| | GATCCGCGTACCGGTTTCGCGAATCTGTTCATTACCAAGGGTCTGACCAATGTTGAGAAGAAGAAGGAGTTTTTCGGCAAGTTCG | |
| | ACTCGATTCGTTATGATGCGACCGAGTCTTGTTTCGTTTTTTCCTTCGACTACGCAAAGATTTGTGACAATGCTGATTACAAGAAGA | |
| | AATGGGATGTTTACACCCGTGGTACTCGTCTCGTTTACAACAAGACCGAACGCAAGAACGTGAGCGTCAACCCAACCGAAGAG | |
| | TTGCAGTGTGTTTTTGATGAGTTCGGCATCAAGTGGAATACCGGCGAAGACCTGATTGAATCTATTAGCCTGATCCCGGCGGAAA | |
| | AGTCCAACGCGAAGTTCTTCGACGTTTTGTTGCGTATGTTTAACGCGACTCTTCAGATGAGAAACAGCGTGCCGAATACCGACAC | |
| | CGACTACTTGGTCAGCCCGGTGAAAGCTGAAGACGGCAGCTTTTTCGACAGCCGCGAGGAGTTTAAGAAAGGTGGCGATGCC | |
| | CGCCTGCCGATAGACTGCGATGCTAATGGTGCGTACCATATTGCCCTGAAAGGCTTGTACTTGTTGTTGAATGATTTCAACCGTGA | |
| | TAACAAGGGCGTCATCCAAAACATCTCTAACAAGGACTGGTTTAAATTCGTGCAGGAGAAAGTTTACAAAGACTAA | |
| 805 | ATGGCTAGGATAATTGATGAATTTTGTGGACAGATGAATGGATACAGCCGTTCCATCACGCTGCGTAACCGCCTGGTGCCGATCG | K549E |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCGAGCTGAACTTCGATTGCCCGACTCTGGCTAATGGCTGGGATG | |
| | AGAATCAAGAGTCCAGCAACGATGCAATTATCCTGAGAAAGGACGGCAAATACTATCTGGGCATTTACAACCCGAACAACAAAC | |
| | CGAAATTCGCGAAGAAGGACAGCATCGTGGGTGATTGCTATGAAAAAATGGCATACAAGCAAATCGCACTGCCGATGGGTTTGG | |
| | GTGCATTTGTGCGCAAATGCTTTGGCACCGCTCAGAAATACGGTTGGGGTTGCCCGGAGAATTGCCTGAACAGCGAGGGTAAA | |
| | ATTATCATCAAAGACGAGGAGGCGAAGGGTAATCTGGAGGCAATCATCGACTGTTACAAAGATTTTCTGAACAAGTACGAAAAGG | |
| | ACGGCTTCAAGTACAAGGACTACAATTTTTCCTTCCTGGACTCTGCCAGCTATGAAAAACTGTCTGATTTCTTCAACGACGTTAAA | |
| | CCGCAGGGCTATAAGCTGAGCTTTACGTCCATCCCACTGTCAGAGATCGATAAGATGATTGATGAAGGTAAACTGTTTTTGTTTCA | |
| | GATATATAACAAAGACTTCGCGAAGAAGGCGACGGGGAAGAAGAACCTGCATACCCTTTATTGGGAAAACCTATTCTCAGTCGA | |
| | GAATCTGCAAGATGTGGTGTTGAAGCTCAATGGCGAAGCCGAGCTGTTCTGGCGTGAGGCTAGCATCAAAAAGGACAAGGTGA | |
| | TCGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 806 | ATGGCTAGGATAATTGATGAATTTTGTGGACAGATGAATGGATACAGCCGTTCCATCACGCTGCGTAACCGCCTGGTGCCGATCG | K579I |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCATATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 807 | ATGGCTAGGATAATTGATGAATTTTGTGGACAGATGAATGGATACAGCCGTTCCATCACGCTGCGTAACCGCCTGGTGCCGATCG | K509E |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGGAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 808 | ATGGCTAGGATAATTGATGAATTTTGTGGACAGATGAATGGATACAGCCGTTCCATCACGCTGCGTAACCGCCTGGTGCCGATCG | K595T |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGACGAAGGACAGCATCGTGGGTGATTGCTATGAAAAAATGGCATACAAGCAAATCGCACTGCCGATGGGTTTGG | |
| | GTGCATTTGTGCGCAAATGCTTTGGCACCGCTCAGAAATACGGTTGGGGTTGCCCGGAGAATTGCCTGAACAGCGAGGGTAAA | |
| | ATTATCATCAAAGACGAGGAGGCGAAGGGTAATCTGGAGGCAATCATCGACTGTTACAAAGATTTTCTGAACAAGTACGAAAAGG | |
| | ACGGCTTCAAGTACAAGGACTACAATTTTTCCTTCCTGGACTCTGCCAGCTATGAAAAACTGTCTGATTTCTTCAACGACGTTAAA | |
| | CCGCAGGGCTATAAGCTGAGCTTTACGTCCATCCCACTGTCAGAGATCGATAAGATGATTGATGAAGGTAAACTGTTTTTGTTTCA | |
| | GATATATAACAAAGACTTCGCGAAGAAGGCGACGGGGAAGAAGAACCTGCATACCCTTTATTGGGAAAACCTATTCTCAGTCGA | |
| | GAATCTGCAAGATGTGGTGTTGAAGCTCAATGGCGAAGCCGAGCTGTTCTGGCGTGAGGCTAGCATCAAAAAGGACAAGGTGA | |
| | TCGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 809 | ATGGCTAGGATAATTGATGAATTTTGTGGACAGATGAATGGATACAGCCGTTCCATCACGCTGCGTAACCGCCTGGTGCCGATCG | V501D |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGACGATTATCGTGGCG | |
| | AAGAGCAGAAAGACTCAACCTTTTATAGCAAACACGATGAAATCCTGACCGATCTTTCTCAGATCGTCCCATTGTATAATAAAGTC | |
| | CGTAACTTTGTTACCAAAAAACTGGGTGAGAGCAAAAAGATCAAGCTGAACTTCGATTGCCCGACTCTGGCTAATGGCTGGGAT | |
| | GAGAATCAAGAGTCCAGCAACGATGCAATTATCCTGAGAAAGGACGGCAAATACTATCTGGGCATTTACAACCCGAACAACAAA | |
| | CCGAAATTCGCGAAGAAGGACAGCATCGTGGGTGATTGCTATGAAAAAATGGCATACAAGCAAATCGCACTGCCGATGGGTTTG | |
| | GGTGCATTTGTGCGCAAATGCTTTGGCACCGCTCAGAAATACGGTTGGGGTTGCCCGGAGAATTGCCTGAACAGCGAGGGTAA | |
| | AATTATCATCAAAGACGAGGAGGCGAAGGGTAATCTGGAGGCAATCATCGACTGTTACAAAGATTTTCTGAACAAGTACGAAAAG | |
| | GACGGCTTCAAGTACAAGGACTACAATTTTTCCTTCCTGGACTCTGCCAGCTATGAAAAACTGTCTGATTTCTTCAACGACGTTAA | |
| | ACCGCAGGGCTATAAGCTGAGCTTTACGTCCATCCCACTGTCAGAGATCGATAAGATGATTGATGAAGGTAAACTGTTTTTGTTTC | |
| | AGATATATAACAAAGACTTCGCGAAGAAGGCGACGGGGAAGAAGAACCTGCATACCCTTTATTGGGAAAACCTATTCTCAGTCG | |
| | AGAATCTGCAAGATGTGGTGTTGAAGCTCAATGGCGAAGCCGAGCTGTTCTGGCGTGAGGCTAGCATCAAAAAGGACAAGGTG | |
| | ATCGTTCACAAAAAAGGTTCTATTCTGGTGAATCGTACCACCACGGATGGTAAGAGCATCCCGGAGGCCATTTATCAAGAAATCT | |
| | ATCAATTAAAAAACAAAATGGCGGATTCCATCAGCGACGAAGCGAAACGCCTTCTAGAGTCCGGCACCGTTGTCTGTAAAGTCG | |
| | CCACTCATGATATCGTTAAGGATAAACATTTTACTGAAAACACGTATCTGTTCCATTGTCCGATTACCATGAACTTCAAGGCAAAAG | |
| | ACCGTACCAATAAGGAATTTAACAATCATGTTCTGGAGGTTCTTAACAAGAATCCGGATATTAAGGTGATCGGCTTAGACCGTGGC | |
| | GAACGTCATCTGCTGTACCTGAGCCTGATCAATCAGAAGGGTGAAATTGAGTGCCAGAAAACGCTGAACCTAGTTGAGCAAGTT | |
| | CGTAATGATAAGACCGTTTCCGTTAATTATCATGAAAAACTGGTGCACAAAGAAGGCAGCCGTGATGCTGCGAGAAAAAACTGGC | |
| | AGACCATTGGCAATATTAAAGAGCTGAAGGAGGGCTACCTGTCGGCGGTGGTGCACGAGATTGCGAGTCTGATGGTAAAGCAC | |
| | AATGCGATCGTTGTAATGGAAGACCTGAATTTCGGCTTTAAGCGCGGTCGTTTTGCTGTGGAGCGTCAGATCTACCAAAAGTTCG | |
| | AAAACATGCTGATTGAGAAGCTGAACTATCTGGTTTTTAAAGACCGTAAAGTGACGGAGCCGGGTGGCGTTCTGAACGCGTACC | |
| | AGTTGGCGAACAAGAGCGCGAAGGTGACAGATGTTTATAAACAATGCGGTTGGCTGTTCTATATCCCGGCAGCGTACACCAGCA | |
| | AAATTGATCCGCGTACCGGTTTCGCGAATCTGTTCATTACCAAGGGTCTGACCAATGTTGAGAAGAAGAAGGAGTTTTTCGGCAA | |
| | GTTCGACTCGATTCGTTATGATGCGACCGAGTCTTGTTTCGTTTTTTCCTTCGACTACGCAAAGATTTGTGACAATGCTGATTACAA | |
| | GAAGAAATGGGATGTTTACACCCGTGGTACTCGTCTCGTTTACAACAAGACCGAACGCAAGAACGTGAGCGTCAACCCAACCG | |
| | AAGAGTTGCAGTGTGTTTTTGATGAGTTCGGCATCAAGTGGAATACCGGCGAAGACCTGATTGAATCTATTAGCCTGATCCCGGC | |
| | GGAAAAGTCCAACGCGAAGTTCTTCGACGTTTTGTTGCGTATGTTTAACGCGACTCTTCAGATGAGAAACAGCGTGCCGAATACC | |
| | GACACCGACTACTTGGTCAGCCCGGTGAAAGCTGAAGACGGCAGCTTTTTCGACAGCCGCGAGGAGTTTAAGAAAGGTGGCG | |
| | ATGCCCGCCTGCCGATAGACTGCGATGCTAATGGTGCGTACCATATTGCCCTGAAAGGCTTGTACTTGTTGTTGAATGATTTCAAC | |
| | CGTGATAACAAGGGCGTCATCCAAAACATCTCTAACAAGGACTGGTTTAAATTCGTGCAGGAGAAAGTTTACAAAGACTAA | |
| 810 | ATGGCTAGGATAATTGATGAATTTTGTGGACAGATGAATGGATACAGCCGTTCCATCACGCTGCGTAACCGCCTGGTGCCGATCG | S568I |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCATCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 811 | ATGGCTAGGATAATTGATGAATTTTGTGGACAGATGAATGGATACAGCCGTTCCATCACGCTGCGTAACCGCCTGGTGCCGATCG | H470L |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCTCATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 312 | ATGGCTAGGATAATTGATGAATTTTGTGGACAGATGAATGGATACAGCCGTTCCATCACGCTGCGTAACCGCCTGGTGCCGATCG | G443R |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGAGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 813 | ATGGCTAGGATAATTGATGAATTTTGTGGACAGATGAATGGATACAGCCGTTCCATCACGCTGCGTAACCGCCTGGTGCCGATCG | P591S |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAATC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 814 | ATGGCTAGGATAATTGATGAATTTTGTGGACAGATGAATGGATACAGCCGTTCCATCACGCTGCGTAACCGCCTGGTGCCGATCG | K516T |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCACACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 815 | ATGGCTAGGATAATTGATGAATTTTGTGGACAGATGAATGGATACAGCCGTTCCATCACGCTGCGTAACCGCCTGGTGCCGATCG | E383V |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGTGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 816 | ATGGCTAGGATAATTGATGAATTTTGTGGACAGATGAATGGATACAGCCGTTCCATCACGCTGCGTAACCGCCTGGTGCCGATCG | L474Q |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCAGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 817 | ATGGCTAGGATAATTGATGAATTTTGTGGACAGATGAATGGATACAGCCGTTCCATCACGCTGCGTAACCGCCTGGTGCCGATCG | H470N |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACAACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 818 | ATGGCTAGGATAATTGATGAATTTTGTGGACAGATGAATGGATACAGCCGTTCCATCACGCTGCGTAACCGCCTGGTGCCGA | E402V |
| | TCGGCAAGACCGAGGAAAATCTGAAACAGTTTCTGGAGAAGGACTTAGAGCGCGCAACTGCGTACCCGGACATCAAAAAC | |
| | CTGATTGACGCTATCCATCGTAATGTCATTGAGGACACCTTAAGCAAAGTGGCCCTGAACTGGAACGAAATCTTTAATATCCT | |
| | CGCGACGTACCAGAACGAAAAAGATAAAAAGAAAAAGGCGGCGATCAAAAAGGACCTGGAGAAGCTCCAAAGCGGTGCA | |
| | CGTAAAAAGATTGTTGAGGCATTCAAAAAGAACCCGGATTTTGAAAAACTGTTCAAAGAGGGTCTGTTTAAAGAGTTGCTGC | |
| | CTGAACTGATCAAAAGCGCACCGGTTGATGAAATCGCGGTTAAAACCAAGGCGCTGGAATGCTTCAACCGTTTTAGCACCT | |
| | ATTTCACGGGTTTCCACGACAATCGCAAAAACATGTACTCGGAAGAAGCTAAATCTACCGCAATTAGCTATCGCATCGTGAA | |
| | CGAAAATTTCCCGAAATTTTTTGCCAATATCAAGTTGTTCAACTATCTGAAAGAGCACTTTCCGCGAATCATCATTGATACCGA | |
| | AGAGTCCCTGAAAGACTATTTGAAGGGCAAAAAGCTGGATTCTGTATTTAGCATTGACGGCTTTAACAGCGTGCTGGCGCA | |
| | GAGCGGGATTGATTTCTATAACACCGTGATAGGCGGTATTTCAGGTGAGGCCGGCACCAAAAAAACGCAGGGTCTTAACGA | |
| | GAAGATTAACTTAGCGCGCCAGCAATTATCCAAGGAGGAGAAGAACAAGCTGCGCGGTAAAATGGTGGTGCTGTTTAAACA | |
| | GATCTTGTCTGATCGTGAAACCAGCAGCTTTATCCCAGTGGGTTTTGCCAACAAGGAGGAGGTGTACTCTACGGTGAAAGA | |
| | ATTCAACAACTCGATTGCCGAAAAGGCGGTGTCCAAGGTGCGTGATCTATTCCTGCACCGCGAAGAATTCACGCTAAACGA | |
| | GATTTTCGTCCCGGCAAAGAGCTTGACCGACTTCAGTCAGGCCATCTTCGGATCCTGGTCTATTTTGAGTGAGGGCCTGTT | |
| | CCTGCTGGAGAAGGACAGCATGAAAAAGGCTCTAAGCGAATCCCAAGAAGAAAAGATCAATAAGGTGATTGCGAAAAAAGA | |
| | CTGCTCCTTTACCGAACTACAACTGGCGTACGAGCGCTACTGCACCGAGCACAACTTGCCGGTTGAAAAATTCTGCAAAGA | |
| | CTACTTCGACATCGTCGATTACAGGGGAAACGGTGCGAAAAGCGAAAAAACAAAAGTGAGCATCCTGAGCGAAATCCTGG | |
| | AAACCTTTTTACAATTGGACTTTGACCACATTCAGGACCTGCAACAAGAAAAGAACGCTGCGATTCCTATCAAAGCATATCTT | |
| | GACGAAGTTCAGAATTTGTACCACCACCTGAAGTTGGTCGATTATCGTGGCGAAGAGCAGAAAGACTCAACCTTTTATAGCA | |
| | AACACGATGAAATCCTGACCGATCTTTCTCAGATCGTCCCATTGTATAATAAAGTCCGTAACTTTGTTACCAAAAAACTGGGT | |
| | GAGAGCAAAAAGATCAAGCTGAACTTCGATTGCCCGACTCTGGCTAATGGCTGGGATGAGAATCAAGAGTCCAGCAACGAT | |
| | GCAATTATCCTGAGAAAGGACGGCAAATACTATCTGGGCATTTACAACCCGAACAACAAACCGAAATTCGCGAAGAAGGAC | |
| | AGCATCGTGGGTGATTGCTATGAAAAAATGGCATACAAGCAAATCGCACTGCCGATGGGTTTGGGTGCATTTGTGCGCAAA | |
| | TGCTTTGGCACCGCTCAGAAATACGGTTGGGGTTGCCCGGAGAATTGCCTGAACAGCGAGGGTAAAATTATCATCAAAGAC | |
| | GAGGAGGCGAAGGGTAATCTGGAGGCAATCATCGACTGTTACAAAGATTTTCTGAACAAGTACGAAAAGGACGGCTTCAAG | |
| | TACAAGGACTACAATTTTTCCTTCCTGGACTCTGCCAGCTATGAAAAACTGTCTGATTTCTTCAACGACGTTAAACCGCAGG | |
| | GCTATAAGCTGAGCTTTACGTCCATCCCACTGTCAGAGATCGATAAGATGATTGATGAAGGTAAACTGTTTTTGTTTCAGATA | |
| | TATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGT | |
| | GATCGTTCACAAAAAAGGTTCTATTCTGGTGAATCGTACCACCACGGATGGTAAGAGCATCCCGGAGGCCATTTATCAAGAA | |
| | ATCTATCAATTAAAAAACAAAATGGCGGATTCCATCAGCGACGAAGCGAAACGCCTTCTAGAGTCCGGCACCGTTGTCTGTA | |
| | AAGTCGCCACTCATGATATCGTTAAGGATAAACATTTTACTGAAAACACGTATCTGTTCCATTGTCCGATTACCATGAACTTCA | |
| | AGGCAAAAGACCGTACCAATAAGGAATTTAACAATCATGTTCTGGAGGTTCTTAACAAGAATCCGGATATTAAGGTGATCGG | |
| | CTTAGACCGTGGCGAACGTCATCTGCTGTACCTGAGCCTGATCAATCAGAAGGGTGAAATTGAGTGCCAGAAAACGCTGAA | |
| | CCTAGTTGAGCAAGTTCGTAATGATAAGACCGTTTCCGTTAATTATCATGAAAAACTGGTGCACAAAGAAGGCAGCCGTGAT | |
| | GCTGCGAGAAAAAACTGGCAGACCATTGGCAATATTAAAGAGCTGAAGGAGGGCTACCTGTCGGCGGTGGTGCACGAGAT | |
| | TGCGAGTCTGATGGTAAAGCACAATGCGATCGTTGTAATGGAAGACCTGAATTTCGGCTTTAAGCGCGGTCGTTTTGCTGT | |
| | GGAGCGTCAGATCTACCAAAAGTTCGAAAACATGCTGATTGAGAAGCTGAACTATCTGGTTTTTAAAGACCGTAAAGTGACG | |
| | GAGCCGGGTGGCGTTCTGAACGCGTACCAGTTGGCGAACAAGAGCGCGAAGGTGACAGATGTTTATAAACAATGCGGTTG | |
| | GCTGTTCTATATCCCGGCAGCGTACACCAGCAAAATTGATCCGCGTACCGGTTTCGCGAATCTGTTCATTACCAAGGGTCTG | |
| | ACCAATGTTGAGAAGAAGAAGGAGTTTTTCGGCAAGTTCGACTCGATTCGTTATGATGCGACCGAGTCTTGTTTCGTTTTTT | |
| | CCTTCGACTACGCAAAGATTTGTGACAATGCTGATTACAAGAAGAAATGGGATGTTTACACCCGTGGTACTCGTCTCGTTTA | |
| | CAACAAGACCGAACGCAAGAACGTGAGCGTCAACCCAACCGAAGAGTTGCAGTGTGTTTTTGATGAGTTCGGCATCAAGT | |
| | GGAATACCGGCGAAGACCTGATTGAATCTATTAGCCTGATCCCGGCGGAAAAGTCCAACGCGAAGTTCTTCGACGTTTTGT | |
| | TGCGTATGTTTAACGCGACTCTTCAGATGAGAAACAGCGTGCCGAATACCGACACCGACTACTTGGTCAGCCCGGTGAAAG | |
| | CTGAAGACGGCAGCTTTTTCGACAGCCGCGAGGAGTTTAAGAAAGGTGGCGATGCCCGCCTGCCGATAGACTGCGATGC | |
| | TAATGGTGCGTACCATATTGCCCTGAAAGGCTTGTACTTGTTGTTGAATGATTTCAACCGTGATAACAAGGGCGTCATCCAAA | |
| | ACATCTCTAACAAGGACTGGTTTAAATTCGTGCAGGAGAAAGTTTACAAAGACTAA | |
| 819 | ATGGCTAGGATAATTGATGAATTTTGTGGACAGATGAATGGATACAGCCGTTCCATCACGCTGCGTAACCGCCTGGTGCCGATCG | Q465R |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACGATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 820 | ATGGCTAGGATAATTGATGAATTTTGTGGACAGATGAATGGATACAGCCGTTCCATCACGCTGCGTAACCGCCTGGTGCCGATCG | F537I |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACATTGTTACCAAAAAACTGGGTGAGAGCAAAAAGATCAAGCTGAACTTCGATTGCCCGACTCTGGCTAATGGCTGGGATG | |
| | AGAATCAAGAGTCCAGCAACGATGCAATTATCCTGAGAAAGGACGGCAAATACTATCTGGGCATTTACAACCCGAACAACAAAC | |
| | CGAAATTCGCGAAGAAGGACAGCATCGTGGGTGATTGCTATGAAAAAATGGCATACAAGCAAATCGCACTGCCGATGGGTTTGG | |
| | GTGCATTTGTGCGCAAATGCTTTGGCACCGCTCAGAAATACGGTTGGGGTTGCCCGGAGAATTGCCTGAACAGCGAGGGTAAA | |
| | ATTATCATCAAAGACGAGGAGGCGAAGGGTAATCTGGAGGCAATCATCGACTGTTACAAAGATTTTCTGAACAAGTACGAAAAGG | |
| | ACGGCTTCAAGTACAAGGACTACAATTTTTCCTTCCTGGACTCTGCCAGCTATGAAAAACTGTCTGATTTCTTCAACGACGTTAAA | |
| | CCGCAGGGCTATAAGCTGAGCTTTACGTCCATCCCACTGTCAGAGATCGATAAGATGATTGATGAAGGTAAACTGTTTTTGTTTCA | |
| | GATATATAACAAAGACTTCGCGAAGAAGGCGACGGGGAAGAAGAACCTGCATACCCTTTATTGGGAAAACCTATTCTCAGTCGA | |
| | GAATCTGCAAGATGTGGTGTTGAAGCTCAATGGCGAAGCCGAGCTGTTCTGGCGTGAGGCTAGCATCAAAAAGGACAAGGTGA | |
| | TCGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 821 | ATGGCTAGGATAATTGATGAATTTTGTGGACAGATGAATGGATACAGCCGTTCCATCACGCTGCGTAACCGCCTGGTGCCGATCG | K745M |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGATGAAGAACCTGCATACCCTTTATTGGGAAAACCTATTCTCAGTCGAGA | |
| | ATCTGCAAGATGTGGTGTTGAAGCTCAATGGCGAAGCCGAGCTGTTCTGGCGTGAGGCTAGCATCAAAAAGGACAAGGTGATC | |
| | GTTCACAAAAAAGGTTCTATTCTGGTGAATCGTACCACCACGGATGGTAAGAGCATCCCGGAGGCCATTTATCAAGAAATCTATC | |
| | AATTAAAAAACAAAATGGCGGATTCCATCAGCGACGAAGCGAAACGCCTTCTAGAGTCCGGCACCGTTGTCTGTAAAGTCGCC | |
| | ACTCATGATATCGTTAAGGATAAACATTTTACTGAAAACACGTATCTGTTCCATTGTCCGATTACCATGAACTTCAAGGCAAAAGAC | |
| | CGTACCAATAAGGAATTTAACAATCATGTTCTGGAGGTTCTTAACAAGAATCCGGATATTAAGGTGATCGGCTTAGACCGTGGCGA | |
| | ACGTCATCTGCTGTACCTGAGCCTGATCAATCAGAAGGGTGAAATTGAGTGCCAGAAAACGCTGAACCTAGTTGAGCAAGTTCGT | |
| | AATGATAAGACCGTTTCCGTTAATTATCATGAAAAACTGGTGCACAAAGAAGGCAGCCGTGATGCTGCGAGAAAAAACTGGCAG | |
| | ACCATTGGCAATATTAAAGAGCTGAAGGAGGGCTACCTGTCGGCGGTGGTGCACGAGATTGCGAGTCTGATGGTAAAGCACAAT | |
| | GCGATCGTTGTAATGGAAGACCTGAATTTCGGCTTTAAGCGCGGTCGTTTTGCTGTGGAGCGTCAGATCTACCAAAAGTTCGAAA | |
| | ACATGCTGATTGAGAAGCTGAACTATCTGGTTTTTAAAGACCGTAAAGTGACGGAGCCGGGTGGCGTTCTGAACGCGTACCAGT | |
| | TGGCGAACAAGAGCGCGAAGGTGACAGATGTTTATAAACAATGCGGTTGGCTGTTCTATATCCCGGCAGCGTACACCAGCAAA | |
| | ATTGATCCGCGTACCGGTTTCGCGAATCTGTTCATTACCAAGGGTCTGACCAATGTTGAGAAGAAGAAGGAGTTTTTCGGCAAGT | |
| | TCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 822 | ATGGCTAGGATAATTGATGAATTTTGTGGACAGATGAATGGATACAGCCGTTCCATCACGCTGCGTAACCGCCTGGTGCCGATCG | T838S |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCTCCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 823 | ATGGCTAGGATAATTGATGAATTTTGTGGACAGATGAATGGATACAGCCGTTCCATCACGCTGCGTAACCGCCTGGTGCCGATCG | N735I |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATATCAAAGACTTCGCGAAGAAGGCGACGGGGAAGAAGAACCTGCATACCCTTTATTGGGAAAACCTATTCTCAGTCGAGA | |
| | ATCTGCAAGATGTGGTGTTGAAGCTCAATGGCGAAGCCGAGCTGTTCTGGCGTGAGGCTAGCATCAAAAAGGACAAGGTGATC | |
| | GTTCACAAAAAAGGTTCTATTCTGGTGAATCGTACCACCACGGATGGTAAGAGCATCCCGGAGGCCATTTATCAAGAAATCTATC | |
| | AATTAAAAAACAAAATGGCGGATTCCATCAGCGACGAAGCGAAACGCCTTCTAGAGTCCGGCACCGTTGTCTGTAAAGTCGCC | |
| | ACTCATGATATCGTTAAGGATAAACATTTTACTGAAAACACGTATCTGTTCCATTGTCCGATTACCATGAACTTCAAGGCAAAAGAC | |
| | CGTACCAATAAGGAATTTAACAATCATGTTCTGGAGGTTCTTAACAAGAATCCGGATATTAAGGTGATCGGCTTAGACCGTGGCGA | |
| | ACGTCATCTGCTGTACCTGAGCCTGATCAATCAGAAGGGTGAAATTGAGTGCCAGAAAACGCTGAACCTAGTTGAGCAAGTTCGT | |
| | AATGATAAGACCGTTTCCGTTAATTATCATGAAAAACTGGTGCACAAAGAAGGCAGCCGTGATGCTGCGAGAAAAAACTGGCAG | |
| | ACCATTGGCAATATTAAAGAGCTGAAGGAGGGCTACCTGTCGGCGGTGGTGCACGAGATTGCGAGTCTGATGGTAAAGCACAAT | |
| | GCGATCGTTGTAATGGAAGACCTGAATTTCGGCTTTAAGCGCGGTCGTTTTGCTGTGGAGCGTCAGATCTACCAAAAGTTCGAAA | |
| | ACATGCTGATTGAGAAGCTGAACTATCTGGTTTTTAAAGACCGTAAAGTGACGGAGCCGGGTGGCGTTCTGAACGCGTACCAGT | |
| | TGGCGAACAAGAGCGCGAAGGTGACAGATGTTTATAAACAATGCGGTTGGCTGTTCTATATCCCGGCAGCGTACACCAGCAAA | |
| | ATTGATCCGCGTACCGGTTTCGCGAATCTGTTCATTACCAAGGGTCTGACCAATGTTGAGAAGAAGAAGGAGTTTTTCGGCAAGT | |
| | TCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 824 | ATGGCTAGGATAATTGATGAATTTTGTGGACAGATGAATGGATACAGCCGTTCCATCACGCTGCGTAACCGCCTGGTGCCGATCG | I723V |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGGTTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 825 | ATGGCTAGGATAATTGATGAATTTTGTGGACAGATGAATGGATACAGCCGTTCCATCACGCTGCGTAACCGCCTGGTGCCGA | K821I |
| | TCGGCAAGACCGAGGAAAATCTGAAACAGTTTCTGGAGAAGGACTTAGAGCGCGCAACTGCGTACCCGGACATCAAAAAC | |
| | CTGATTGACGCTATCCATCGTAATGTCATTGAGGACACCTTAAGCAAAGTGGCCCTGAACTGGAACGAAATCTTTAATATCCT | |
| | CGCGACGTACCAGAACGAAAAAGATAAAAAGAAAAAGGCGGCGATCAAAAAGGACCTGGAGAAGCTCCAAAGCGGTGCA | |
| | CGTAAAAAGATTGTTGAGGCATTCAAAAAGAACCCGGATTTTGAAAAACTGTTCAAAGAGGGTCTGTTTAAAGAGTTGCTGC | |
| | CTGAACTGATCAAAAGCGCACCGGTTGATGAAATCGCGGTTAAAACCAAGGCGCTGGAATGCTTCAACCGTTTTAGCACCT | |
| | ATTTCACGGGTTTCCACGACAATCGCAAAAACATGTACTCGGAAGAAGCTAAATCTACCGCAATTAGCTATCGCATCGTGAA | |
| | CGAAAATTTCCCGAAATTTTTTGCCAATATCAAGTTGTTCAACTATCTGAAAGAGCACTTTCCGCGAATCATCATTGATACCGA | |
| | AGAGTCCCTGAAAGACTATTTGAAGGGCAAAAAGCTGGATTCTGTATTTAGCATTGACGGCTTTAACAGCGTGCTGGCGCA | |
| | GAGCGGGATTGATTTCTATAACACCGTGATAGGCGGTATTTCAGGTGAGGCCGGCACCAAAAAAACGCAGGGTCTTAACGA | |
| | GAAGATTAACTTAGCGCGCCAGCAATTATCCAAGGAGGAGAAGAACAAGCTGCGCGGTAAAATGGTGGTGCTGTTTAAACA | |
| | GATCTTGTCTGATCGTGAAACCAGCAGCTTTATCCCAGTGGGTTTTGCCAACAAGGAGGAGGTGTACTCTACGGTGAAAGA | |
| | ATTCAACAACTCGATTGCCGAAAAGGCGGTGTCCAAGGTGCGTGATCTATTCCTGCACCGCGAAGAATTCACGCTAAACGA | |
| | GATTTTCGTCCCGGCAAAGAGCTTGACCGACTTCAGTCAGGCCATCTTCGGATCCTGGTCTATTTTGAGTGAGGGCCTGTT | |
| | CCTGCTGGAGAAGGACAGCATGAAAAAGGCTCTAAGCGAATCCCAAGAAGAAAAGATCAATAAGGAGATTGCGAAAAAAGA | |
| | CTGCTCCTTTACCGAACTACAACTGGCGTACGAGCGCTACTGCACCGAGCACAACTTGCCGGTTGAAAAATTCTGCAAAGA | |
| | CTACTTCGACATCGTCGATTACAGGGGAAACGGTGCGAAAAGCGAAAAAACAAAAGTGAGCATCCTGAGCGAAATCCTGG | |
| | AAACCTTTTTACAATTGGACTTTGACCACATTCAGGACCTGCAACAAGAAAAGAACGCTGCGATTCCTATCAAAGCATATCTT | |
| | GACGAAGTTCAGAATTTGTACCACCACCTGAAGTTGGTCGATTATCGTGGCGAAGAGCAGAAAGACTCAACCTTTTATAGCA | |
| | AACACGATGAAATCCTGACCGATCTTTCTCAGATCGTCCCATTGTATAATAAAGTCCGTAACTTTGTTACCAAAAAACTGGGT | |
| | GAGAGCAAAAAGATCAAGCTGAACTTCGATTGCCCGACTCTGGCTAATGGCTGGGATGAGAATCAAGAGTCCAGCAACGAT | |
| | GCAATTATCCTGAGAAAGGACGGCAAATACTATCTGGGCATTTACAACCCGAACAACAAACCGAAATTCGCGAAGAAGGAC | |
| | AGCATCGTGGGTGATTGCTATGAAAAAATGGCATACAAGCAAATCGCACTGCCGATGGGTTTGGGTGCATTTGTGCGCAAA | |
| | TGCTTTGGCACCGCTCAGAAATACGGTTGGGGTTGCCCGGAGAATTGCCTGAACAGCGAGGGTAAAATTATCATCAAAGAC | |
| | GAGGAGGCGAAGGGTAATCTGGAGGCAATCATCGACTGTTACAAAGATTTTCTGAACAAGTACGAAAAGGACGGCTTCAAG | |
| | TACAAGGACTACAATTTTTCCTTCCTGGACTCTGCCAGCTATGAAAAACTGTCTGATTTCTTCAACGACGTTAAACCGCAGG | |
| | GCTATAAGCTGAGCTTTACGTCCATCCCACTGTCAGAGATCGATAAGATGATTGATGAAGGTAAACTGTTTTTGTTTCAGATA | |
| | TATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGT | |
| | GATCGTTCACAAAAAAGGTTCTATTCTGGTGAATCGTACCACCACGGATGGTAAGAGCATCCCGGAGGCCATTTATCAAGAA | |
| | ATCTATCAATTAAAAAACATAATGGCGGATTCCATCAGCGACGAAGCGAAACGCCTTCTAGAGTCCGGCACCGTTGTCTGTA | |
| | AAGTCGCCACTCATGATATCGTTAAGGATAAACATTTTACTGAAAACACGTATCTGTTCCATTGTCCGATTACCATGAACTTCA | |
| | AGGCAAAAGACCGTACCAATAAGGAATTTAACAATCATGTTCTGGAGGTTCTTAACAAGAATCCGGATATTAAGGTGATCGG | |
| | CTTAGACCGTGGCGAACGTCATCTGCTGTACCTGAGCCTGATCAATCAGAAGGGTGAAATTGAGTGCCAGAAAACGCTGAA | |
| | CCTAGTTGAGCAAGTTCGTAATGATAAGACCGTTTCCGTTAATTATCATGAAAAACTGGTGCACAAAGAAGGCAGCCGTGAT | |
| | GCTGCGAGAAAAAACTGGCAGACCATTGGCAATATTAAAGAGCTGAAGGAGGGCTACCTGTCGGCGGTGGTGCACGAGAT | |
| | TGCGAGTCTGATGGTAAAGCACAATGCGATCGTTGTAATGGAAGACCTGAATTTCGGCTTTAAGCGCGGTCGTTTTGCTGT | |
| | GGAGCGTCAGATCTACCAAAAGTTCGAAAACATGCTGATTGAGAAGCTGAACTATCTGGTTTTTAAAGACCGTAAAGTGACG | |
| | GAGCCGGGTGGCGTTCTGAACGCGTACCAGTTGGCGAACAAGAGCGCGAAGGTGACAGATGTTTATAAACAATGCGGTTG | |
| | GCTGTTCTATATCCCGGCAGCGTACACCAGCAAAATTGATCCGCGTACCGGTTTCGCGAATCTGTTCATTACCAAGGGTCTG | |
| | ACCAATGTTGAGAAGAAGAAGGAGTTTTTCGGCAAGTTCGACTCGATTCGTTATGATGCGACCGAGTCTTGTTTCGTTTTTT | |
| | CCTTCGACTACGCAAAGATTTGTGACAATGCTGATTACAAGAAGAAATGGGATGTTTACACCCGTGGTACTCGTCTCGTTTA | |
| | CAACAAGACCGAACGCAAGAACGTGAGCGTCAACCCAACCGAAGAGTTGCAGTGTGTTTTTGATGAGTTCGGCATCAAGT | |
| | GGAATACCGGCGAAGACCTGATTGAATCTATTAGCCTGATCCCGGCGGAAAAGTCCAACGCGAAGTTCTTCGACGTTTTGT | |
| | TGCGTATGTTTAACGCGACTCTTCAGATGAGAAACAGCGTGCCGAATACCGACACCGACTACTTGGTCAGCCCGGTGAAAG | |
| | CTGAAGACGGCAGCTTTTTCGACAGCCGCGAGGAGTTTAAGAAAGGTGGCGATGCCCGCCTGCCGATAGACTGCGATGC | |
| | TAATGGTGCGTACCATATTGCCCTGAAAGGCTTGTACTTGTTGTTGAATGATTTCAACCGTGATAACAAGGGCGTCATCCAAA | |
| | ACATCTCTAACAAGGACTGGTTTAAATTCGTGCAGGAGAAAGTTTACAAAGACTAA | |
| 826 | ATGGCTAGGATAATTGATGAATTTTGTGGACAGATGAATGGATACAGCCGTTCCATCACGCTGCGTAACCGCCTGGTGCCGATCG | K721M |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATATGATGATTGATGAAGGTAAACTGTTTTTGTTTCAGA | |
| | TATATAACAAAGACTTCGCGAAGAAGGCGACGGGGAAGAAGAACCTGCATACCCTTTATTGGGAAAACCTATTCTCAGTCGAGA | |
| | ATCTGCAAGATGTGGTGTTGAAGCTCAATGGCGAAGCCGAGCTGTTCTGGCGTGAGGCTAGCATCAAAAAGGACAAGGTGATC | |
| | GTTCACAAAAAAGGTTCTATTCTGGTGAATCGTACCACCACGGATGGTAAGAGCATCCCGGAGGCCATTTATCAAGAAATCTATC | |
| | AATTAAAAAACAAAATGGCGGATTCCATCAGCGACGAAGCGAAACGCCTTCTAGAGTCCGGCACCGTTGTCTGTAAAGTCGCC | |
| | ACTCATGATATCGTTAAGGATAAACATTTTACTGAAAACACGTATCTGTTCCATTGTCCGATTACCATGAACTTCAAGGCAAAAGAC | |
| | CGTACCAATAAGGAATTTAACAATCATGTTCTGGAGGTTCTTAACAAGAATCCGGATATTAAGGTGATCGGCTTAGACCGTGGCGA | |
| | ACGTCATCTGCTGTACCTGAGCCTGATCAATCAGAAGGGTGAAATTGAGTGCCAGAAAACGCTGAACCTAGTTGAGCAAGTTCGT | |
| | AATGATAAGACCGTTTCCGTTAATTATCATGAAAAACTGGTGCACAAAGAAGGCAGCCGTGATGCTGCGAGAAAAAACTGGCAG | |
| | ACCATTGGCAATATTAAAGAGCTGAAGGAGGGCTACCTGTCGGCGGTGGTGCACGAGATTGCGAGTCTGATGGTAAAGCACAAT | |
| | GCGATCGTTGTAATGGAAGACCTGAATTTCGGCTTTAAGCGCGGTCGTTTTGCTGTGGAGCGTCAGATCTACCAAAAGTTCGAAA | |
| | ACATGCTGATTGAGAAGCTGAACTATCTGGTTTTTAAAGACCGTAAAGTGACGGAGCCGGGTGGCGTTCTGAACGCGTACCAGT | |
| | TGGCGAACAAGAGCGCGAAGGTGACAGATGTTTATAAACAATGCGGTTGGCTGTTCTATATCCCGGCAGCGTACACCAGCAAA | |
| | ATTGATCCGCGTACCGGTTTCGCGAATCTGTTCATTACCAAGGGTCTGACCAATGTTGAGAAGAAGAAGGAGTTTTTCGGCAAGT | |
| | TCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 827 | ATGGCTAGGATAATTGATGAATTTTGTGGACAGATGAATGGATACAGCCGTTCCATCACGCTGCGTAACCGCCTGGTGCCGA | A810V |
| | TCGGCAAGACCGAGGAAAATCTGAAACAGTTTCTGGAGAAGGACTTAGAGCGCGCAACTGCGTACCCGGACATCAAAAAC | |
| | CTGATTGACGCTATCCATCGTAATGTCATTGAGGACACCTTAAGCAAAGTGGCCCTGAACTGGAACGAAATCTTTAATATCCT | |
| | CGCGACGTACCAGAACGAAAAAGATAAAAAGAAAAAGGCGGCGATCAAAAAGGACCTGGAGAAGCTCCAAAGCGGTGCA | |
| | CGTAAAAAGATTGTTGAGGCATTCAAAAAGAACCCGGATTTTGAAAAACTGTTCAAAGAGGGTCTGTTTAAAGAGTTGCTGC | |
| | CTGAACTGATCAAAAGCGCACCGGTTGATGAAATCGCGGTTAAAACCAAGGCGCTGGAATGCTTCAACCGTTTTAGCACCT | |
| | ATTTCACGGGTTTCCACGACAATCGCAAAAACATGTACTCGGAAGAAGCTAAATCTACCGCAATTAGCTATCGCATCGTGAA | |
| | CGAAAATTTCCCGAAATTTTTTGCCAATATCAAGTTGTTCAACTATCTGAAAGAGCACTTTCCGCGAATCATCATTGATACCGA | |
| | AGAGTCCCTGAAAGACTATTTGAAGGGCAAAAAGCTGGATTCTGTATTTAGCATTGACGGCTTTAACAGCGTGCTGGCGCA | |
| | GAGCGGGATTGATTTCTATAACACCGTGATAGGCGGTATTTCAGGTGAGGCCGGCACCAAAAAAACGCAGGGTCTTAACGA | |
| | GAAGATTAACTTAGCGCGCCAGCAATTATCCAAGGAGGAGAAGAACAAGCTGCGCGGTAAAATGGTGGTGCTGTTTAAACA | |
| | GATCTTGTCTGATCGTGAAACCAGCAGCTTTATCCCAGTGGGTTTTGCCAACAAGGAGGAGGTGTACTCTACGGTGAAAGA | |
| | ATTCAACAACTCGATTGCCGAAAAGGCGGTGTCCAAGGTGCGTGATCTATTCCTGCACCGCGAAGAATTCACGCTAAACGA | |
| | GATTTTCGTCCCGGCAAAGAGCTTGACCGACTTCAGTCAGGCCATCTTCGGATCCTGGTCTATTTTGAGTGAGGGCCTGTT | |
| | CCTGCTGGAGAAGGACAGCATGAAAAAGGCTCTAAGCGAATCCCAAGAAGAAAAGATCAATAAGGAGATTGCGAAAAAAGA | |
| | CTGCTCCTTTACCGAACTACAACTGGCGTACGAGCGCTACTGCACCGAGCACAACTTGCCGGTTGAAAAATTCTGCAAAGA | |
| | CTACTTCGACATCGTCGATTACAGGGGAAACGGTGCGAAAAGCGAAAAAACAAAAGTGAGCATCCTGAGCGAAATCCTGG | |
| | AAACCTTTTTACAATTGGACTTTGACCACATTCAGGACCTGCAACAAGAAAAGAACGCTGCGATTCCTATCAAAGCATATCTT | |
| | GACGAAGTTCAGAATTTGTACCACCACCTGAAGTTGGTCGATTATCGTGGCGAAGAGCAGAAAGACTCAACCTTTTATAGCA | |
| | AACACGATGAAATCCTGACCGATCTTTCTCAGATCGTCCCATTGTATAATAAAGTCCGTAACTTTGTTACCAAAAAACTGGGT | |
| | GAGAGCAAAAAGATCAAGCTGAACTTCGATTGCCCGACTCTGGCTAATGGCTGGGATGAGAATCAAGAGTCCAGCAACGAT | |
| | GCAATTATCCTGAGAAAGGACGGCAAATACTATCTGGGCATTTACAACCCGAACAACAAACCGAAATTCGCGAAGAAGGAC | |
| | AGCATCGTGGGTGATTGCTATGAAAAAATGGCATACAAGCAAATCGCACTGCCGATGGGTTTGGGTGCATTTGTGCGCAAA | |
| | TGCTTTGGCACCGCTCAGAAATACGGTTGGGGTTGCCCGGAGAATTGCCTGAACAGCGAGGGTAAAATTATCATCAAAGAC | |
| | GAGGAGGCGAAGGGTAATCTGGAGGCAATCATCGACTGTTACAAAGATTTTCTGAACAAGTACGAAAAGGACGGCTTCAAG | |
| | TACAAGGACTACAATTTTTCCTTCCTGGACTCTGCCAGCTATGAAAAACTGTCTGATTTCTTCAACGACGTTAAACCGCAGG | |
| | GCTATAAGCTGAGCTTTACGTCCATCCCACTGTCAGAGATCGATAAGATGATTGATGAAGGTAAACTGTTTTTGTTTCAGATA | |
| | TATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGT | |
| | GATCGTTCACAAAAAAGGTTCTATTCTGGTGAATCGTACCACCACGGATGGTAAGAGCATCCCGGAGGTCATTTATCAAGAA | |
| | ATCTATCAATTAAAAAACAAAATGGCGGATTCCATCAGCGACGAAGCGAAACGCCTTCTAGAGTCCGGCACCGTTGTCTGTA | |
| | AAGTCGCCACTCATGATATCGTTAAGGATAAACATTTTACTGAAAACACGTATCTGTTCCATTGTCCGATTACCATGAACTTCA | |
| | AGGCAAAAGACCGTACCAATAAGGAATTTAACAATCATGTTCTGGAGGTTCTTAACAAGAATCCGGATATTAAGGTGATCGG | |
| | CTTAGACCGTGGCGAACGTCATCTGCTGTACCTGAGCCTGATCAATCAGAAGGGTGAAATTGAGTGCCAGAAAACGCTGAA | |
| | CCTAGTTGAGCAAGTTCGTAATGATAAGACCGTTTCCGTTAATTATCATGAAAAACTGGTGCACAAAGAAGGCAGCCGTGAT | |
| | GCTGCGAGAAAAAACTGGCAGACCATTGGCAATATTAAAGAGCTGAAGGAGGGCTACCTGTCGGCGGTGGTGCACGAGAT | |
| | TGCGAGTCTGATGGTAAAGCACAATGCGATCGTTGTAATGGAAGACCTGAATTTCGGCTTTAAGCGCGGTCGTTTTGCTGT | |
| | GGAGCGTCAGATCTACCAAAAGTTCGAAAACATGCTGATTGAGAAGCTGAACTATCTGGTTTTTAAAGACCGTAAAGTGACG | |
| | GAGCCGGGTGGCGTTCTGAACGCGTACCAGTTGGCGAACAAGAGCGCGAAGGTGACAGATGTTTATAAACAATGCGGTTG | |
| | GCTGTTCTATATCCCGGCAGCGTACACCAGCAAAATTGATCCGCGTACCGGTTTCGCGAATCTGTTCATTACCAAGGGTCTG | |
| | ACCAATGTTGAGAAGAAGAAGGAGTTTTTCGGCAAGTTCGACTCGATTCGTTATGATGCGACCGAGTCTTGTTTCGTTTTTT | |
| | CCTTCGACTACGCAAAGATTTGTGACAATGCTGATTACAAGAAGAAATGGGATGTTTACACCCGTGGTACTCGTCTCGTTTA | |
| | CAACAAGACCGAACGCAAGAACGTGAGCGTCAACCCAACCGAAGAGTTGCAGTGTGTTTTTGATGAGTTCGGCATCAAGT | |
| | GGAATACCGGCGAAGACCTGATTGAATCTATTAGCCTGATCCCGGCGGAAAAGTCCAACGCGAAGTTCTTCGACGTTTTGT | |
| | TGCGTATGTTTAACGCGACTCTTCAGATGAGAAACAGCGTGCCGAATACCGACACCGACTACTTGGTCAGCCCGGTGAAAG | |
| | CTGAAGACGGCAGCTTTTTCGACAGCCGCGAGGAGTTTAAGAAAGGTGGCGATGCCCGCCTGCCGATAGACTGCGATGC | |
| | TAATGGTGCGTACCATATTGCCCTGAAAGGCTTGTACTTGTTGTTGAATGATTTCAACCGTGATAACAAGGGCGTCATCCAAA | |
| | ACATCTCTAACAAGGACTGGTTTAAATTCGTGCAGGAGAAAGTTTACAAAGACTAA | |
| 828 | ATGGCTAGGATAATTGATGAATTTTGTGGACAGATGAATGGATACAGCCGTTCCATCACGCTGCGTAACCGCCTGGTGCCGATCG | K768M |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGATGCTCAATGGCGAAGCCGAGCTGTTCTGGCGTGAGGCTAGCATCAAAAAGGACAAGGTGATC | |
| | GTTCACAAAAAAGGTTCTATTCTGGTGAATCGTACCACCACGGATGGTAAGAGCATCCCGGAGGCCATTTATCAAGAAATCTATC | |
| | AATTAAAAAACAAAATGGCGGATTCCATCAGCGACGAAGCGAAACGCCTTCTAGAGTCCGGCACCGTTGTCTGTAAAGTCGCC | |
| | ACTCATGATATCGTTAAGGATAAACATTTTACTGAAAACACGTATCTGTTCCATTGTCCGATTACCATGAACTTCAAGGCAAAAGAC | |
| | CGTACCAATAAGGAATTTAACAATCATGTTCTGGAGGTTCTTAACAAGAATCCGGATATTAAGGTGATCGGCTTAGACCGTGGCGA | |
| | ACGTCATCTGCTGTACCTGAGCCTGATCAATCAGAAGGGTGAAATTGAGTGCCAGAAAACGCTGAACCTAGTTGAGCAAGTTCGT | |
| | AATGATAAGACCGTTTCCGTTAATTATCATGAAAAACTGGTGCACAAAGAAGGCAGCCGTGATGCTGCGAGAAAAAACTGGCAG | |
| | ACCATTGGCAATATTAAAGAGCTGAAGGAGGGCTACCTGTCGGCGGTGGTGCACGAGATTGCGAGTCTGATGGTAAAGCACAAT | |
| | GCGATCGTTGTAATGGAAGACCTGAATTTCGGCTTTAAGCGCGGTCGTTTTGCTGTGGAGCGTCAGATCTACCAAAAGTTCGAAA | |
| | ACATGCTGATTGAGAAGCTGAACTATCTGGTTTTTAAAGACCGTAAAGTGACGGAGCCGGGTGGCGTTCTGAACGCGTACCAGT | |
| | TGGCGAACAAGAGCGCGAAGGTGACAGATGTTTATAAACAATGCGGTTGGCTGTTCTATATCCCGGCAGCGTACACCAGCAAA | |
| | ATTGATCCGCGTACCGGTTTCGCGAATCTGTTCATTACCAAGGGTCTGACCAATGTTGAGAAGAAGAAGGAGTTTTTCGGCAAGT | |
| | TCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 829 | ATGGCTAGGATAATTGATGAATTTTGTGGACAGATGAATGGATACAGCCGTTCCATCACGCTGCGTAACCGCCTGGTGCCGATCG | S781R |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTTGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGAATCAAAAAGGACAAGGTGATC | |
| | GTTCACAAAAAAGGTTCTATTCTGGTGAATCGTACCACCACGGATGGTAAGAGCATCCCGGAGGCCATTTATCAAGAAATCTATC | |
| | AATTAAAAAACAAAATGGCGGATTCCATCAGCGACGAAGCGAAACGCCTTCTAGAGTCCGGCACCGTTGTCTGTAAAGTCGCC | |
| | ACTCATGATATCGTTAAGGATAAACATTTTACTGAAAACACGTATCTGTTCCATTGTCCGATTACCATGAACTTCAAGGCAAAAGAC | |
| | CGTACCAATAAGGAATTTAACAATCATGTTCTGGAGGTTCTTAACAAGAATCCGGATATTAAGGTGATCGGCTTAGACCGTGGCGA | |
| | ACGTCATCTGCTGTACCTGAGCCTGATCAATCAGAAGGGTGAAATTGAGTGCCAGAAAACGCTGAACCTAGTTGAGCAAGTTCGT | |
| | AATGATAAGACCGTTTCCGTTAATTATCATGAAAAACTGGTGCACAAAGAAGGCAGCCGTGATGCTGCGAGAAAAAACTGGCAG | |
| | ACCATTGGCAATATTAAAGAGCTGAAGGAGGGCTACCTGTCGGCGGTGGTGCACGAGATTGCGAGTCTGATGGTAAAGCACAAT | |
| | GCGATCGTTGTAATGGAAGACCTGAATTTCGGCTTTAAGCGCGGTCGTTTTGCTGTGGAGCGTCAGATCTACCAAAAGTTCGAAA | |
| | ACATGCTGATTGAGAAGCTGAACTATCTGGTTTTTAAAGACCGTAAAGTGACGGAGCCGGGTGGCGTTCTGAACGCGTACCAGT | |
| | TGGCGAACAAGAGCGCGAAGGTGACAGATGTTTATAAACAATGCGGTTGGCTGTTCTATATCCCGGCAGCGTACACCAGCAAA | |
| | ATTGATCCGCGTACCGGTTTCGCGAATCTGTTCATTACCAAGGGTCTGACCAATGTTGAGAAGAAGAAGGAGTTTTTCGGCAAGT | |
| | TCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 830 | ATGGCTAGGATAATTGATGAATTTTGTGGACAGATGAATGGATACAGCCGTTCCATCACGCTGCGTAACCGCCTGGTGCCGATCG | E725D |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGATGGTAAACTGTTTTTGTTTCAGA | |
| | TATATAACAAAGACTTCGCGAAGAAGGCGACGGGGAAGAAGAACCTGCATACCCTTTATTGGGAAAACCTATTCTCAGTCGAGA | |
| | ATCTGCAAGATGTGGTGTTGAAGCTCAATGGCGAAGCCGAGCTGTTCTGGCGTGAGGCTAGCATCAAAAAGGACAAGGTGATC | |
| | GTTCACAAAAAAGGTTCTATTCTGGTGAATCGTACCACCACGGATGGTAAGAGCATCCCGGAGGCCATTTATCAAGAAATCTATC | |
| | AATTAAAAAACAAAATGGCGGATTCCATCAGCGACGAAGCGAAACGCCTTCTAGAGTCCGGCACCGTTGTCTGTAAAGTCGCC | |
| | ACTCATGATATCGTTAAGGATAAACATTTTACTGAAAACACGTATCTGTTCCATTGTCCGATTACCATGAACTTCAAGGCAAAAGAC | |
| | CGTACCAATAAGGAATTTAACAATCATGTTCTGGAGGTTCTTAACAAGAATCCGGATATTAAGGTGATCGGCTTAGACCGTGGCGA | |
| | ACGTCATCTGCTGTACCTGAGCCTGATCAATCAGAAGGGTGAAATTGAGTGCCAGAAAACGCTGAACCTAGTTGAGCAAGTTCGT | |
| | AATGATAAGACCGTTTCCGTTAATTATCATGAAAAACTGGTGCACAAAGAAGGCAGCCGTGATGCTGCGAGAAAAAACTGGCAG | |
| | ACCATTGGCAATATTAAAGAGCTGAAGGAGGGCTACCTGTCGGCGGTGGTGCACGAGATTGCGAGTCTGATGGTAAAGCACAAT | |
| | GCGATCGTTGTAATGGAAGACCTGAATTTCGGCTTTAAGCGCGGTCGTTTTGCTGTGGAGCGTCAGATCTACCAAAAGTTCGAAA | |
| | ACATGCTGATTGAGAAGCTGAACTATCTGGTTTTTAAAGACCGTAAAGTGACGGAGCCGGGTGGCGTTCTGAACGCGTACCAGT | |
| | TGGCGAACAAGAGCGCGAAGGTGACAGATGTTTATAAACAATGCGGTTGGCTGTTCTATATCCCGGCAGCGTACACCAGCAAA | |
| | ATTGATCCGCGTACCGGTTTCGCGAATCTGTTCATTACCAAGGGTCTGACCAATGTTGAGAAGAAGAAGGAGTTTTTCGGCAAGT | |
| | TCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |
| 831 | ATGGCTAGGATAATTGATGAATTTTGTGGACAGATGAATGGATACAGCCGTTCCATCACGCTGCGTAACCGCCTGGTGCCGATCG | L730S |
| | GCAAGACCGAGGAAAATCTGAAACAGTTTCTGGAGAAGGACTTAGAGCGCGCAACTGCGTACCCGGACATCAAAAACCTGATT | |
| | GACGCTATCCATCGTAATGTCATTGAGGACACCTTAAGCAAAGTGGCCCTGAACTGGAACGAAATCTTTAATATCCTCGCGACGT | |
| | ACCAGAACGAAAAAGATAAAAAGAAAAAGGCGGCGATCAAAAAGGACCTGGAGAAGCTCCAAAGCGGTGCACGTAAAAAGAT | |
| | TGTTGAGGCATTCAAAAAGAACCCGGATTTTGAAAAACTGTTCAAAGAGGGTCTGTTTAAAGAGTTGCTGCCTGAACTGATCAAAA | |
| | GCGCACCGGTTGATGAAATCGCGGTTAAAACCAAGGCGCTGGAATGCTTCAACCGTTTTAGCACCTATTTCACGGGTTTCCACG | |
| | ACAATCGCAAAAACATGTACTCGGAAGAAGCTAAATCTACCGCAATTAGCTATCGCATCGTGAACGAAAATTTCCCGAAATTTTTT | |
| | GCCAATATCAAGTTGTTCAACTATCTGAAAGAGCACTTTCCGCGAATCATCATTGATACCGAAGAGTCCCTGAAAGACTATTTGAA | |
| | GGGCAAAAAGCTGGATTCTGTATTTAGCATTGACGGCTTTAACAGCGTGCTGGCGCAGAGCGGGATTGATTTCTATAACACCGTG | |
| | ATAGGCGGTATTTCAGGTGAGGCCGGCACCAAAAAAACGCAGGGTCTTAACGAGAAGATTAACTTAGCGCGCCAGCAATTATC | |
| | CAAGGAGGAGAAGAACAAGCTGCGCGGTAAAATGGTGGTGCTGTTTAAACAGATCTTGTCTGATCGTGAAACCAGCAGCTTTAT | |
| | CCCAGTGGGTTTTGCCAACAAGGAGGAGGTGTACTCTACGGTGAAAGAATTCAACAACTCGATTGCCGAAAAGGCGGTGTCCA | |
| | AGGTGCGTGATCTATTCCTGCACCGCGAAGAATTCACGCTAAACGAGATTTTCGTCCCGGCAAAGAGCTTGACCGACTTCAGTC | |
| | AGGCCATCTTCGGATCCTGGTCTATTTTGAGTGAGGGCCTGTTCCTGCTGGAGAAGGACAGCATGAAAAAGGCTCTAAGCGAAT | |
| | CCCAAGAAGAAAAGATCAATAAGGAGATTGCGAAAAAAGACTGCTCCTTTACCGAACTACAACTGGCGTACGAGCGCTACTGC | |
| | ACCGAGCACAACTTGCCGGTTGAAAAATTCTGCAAAGACTACTTCGACATCGTCGATTACAGGGGAAACGGTGCGAAAAGCGA | |
| | AAAAACAAAAGTGAGCATCCTGAGCGAAATCCTGGAAACCTTTTTACAATTGGACTTTGACCACATTCAGGACCTGCAACAAGAA | |
| | AAGAACGCTGCGATTCCTATCAAAGCATATCTTGACGAAGTTCAGAATTTGTACCACCACCTGAAGTTGGTCGATTATCGTGGCGA | |
| | AGAGCAGAAAGACTCAACCTTTTATAGCAAACACGATGAAATCCTGACCGATCTTTCTCAGATCGTCCCATTGTATAATAAAGTCC | |
| | GTAACTTTGTTACCAAAAAACTGGGTGAGAGCAAAAAGATCAAGCTGAACTTCGATTGCCCGACTCTGGCTAATGGCTGGGATGA | |
| | GAATCAAGAGTCCAGCAACGATGCAATTATCCTGAGAAAGGACGGCAAATACTATCTGGGCATTTACAACCCGAACAACAAACC | |
| | GAAATTCGCGAAGAAGGACAGCATCGTGGGTGATTGCTATGAAAAAATGGCATACAAGCAAATCGCACTGCCGATGGGTTTGGG | |
| | TGCATTTGTGCGCAAATGCTTTGGCACCGCTCAGAAATACGGTTGGGGTTGCCCGGAGAATTGCCTGAACAGCGAGGGTAAAA | |
| | TTATCATCAAAGACGAGGAGGCGAAGGGTAATCTGGAGGCAATCATCGACTGTTACAAAGATTTTCTGAACAAGTACGAAAAGGA | |
| | CGGCTTCAAGTACAAGGACTACAATTTTTCCTTCCTGGACTCTGCCAGCTATGAAAAACTGTCTGATTTCTTCAACGACGTTAAAC | |
| | CGCAGGGCTATAAGCTGAGCTTTACGTCCATCCCACTGTCAGAGATCGATAAGATGATTGATGAAGGTAAACTGTTTTCGTTTCAG | |
| | ATATATAACAAAGACTTCGCGAAGAAGGCGACGGGGAAGAAGAACCTGCATACCCTTTATTGGGAAAACCTATTCTCAGTCGAG | |
| | AATCTGCAAGATGTGGTGTTGAAGCTCAATGGCGAAGCCGAGCTGTTCTGGCGTGAGGCTAGCATCAAAAAGGACAAGGTGAT | |
| | CGTTCACAAAAAAGGTTCTATTCTGGTGAATCGTACCACCACGGATGGTAAGAGCATCCCGGAGGCCATTTATCAAGAAATCTAT | |
| | CAATTAAAAAACAAAATGGCGGATTCCATCAGCGACGAAGCGAAACGCCTTCTAGAGTCCGGCACCGTTGTCTGTAAAGTCGC | |
| | CACTCATGATATCGTTAAGGATAAACATTTTACTGAAAACACGTATCTGTTCCATTGTCCGATTACCATGAACTTCAAGGCAAAAGA | |
| | CCGTACCAATAAGGAATTTAACAATCATGTTCTGGAGGTTCTTAACAAGAATCCGGATATTAAGGTGATCGGCTTAGACCGTGGCG | |
| | AACGTCATCTGCTGTACCTGAGCCTGATCAATCAGAAGGGTGAAATTGAGTGCCAGAAAACGCTGAACCTAGTTGAGCAAGTTC | |
| | GTAATGATAAGACCGTTTCCGTTAATTATCATGAAAAACTGGTGCACAAAGAAGGCAGCCGTGATGCTGCGAGAAAAAACTGGCA | |
| | GACCATTGGCAATATTAAAGAGCTGAAGGAGGGCTACCTGTCGGCGGTGGTGCACGAGATTGCGAGTCTGATGGTAAAGCACA | |
| | ATGCGATCGTTGTAATGGAAGACCTGAATTTCGGCTTTAAGCGCGGTCGTTTTGCTGTGGAGCGTCAGATCTACCAAAAGTTCGA | |
| | AAACATGCTGATTGAGAAGCTGAACTATCTGGTTTTTAAAGACCGTAAAGTGACGGAGCCGGGTGGCGTTCTGAACGCGTACCA | |
| | GTTGGCGAACAAGAGCGCGAAGGTGACAGATGTTTATAAACAATGCGGTTGGCTGTTCTATATCCCGGCAGCGTACACCAGCAA | |
| | AATTGATCCGCGTACCGGTTTCGCGAATCTGTTCATTACCAAGGGTCTGACCAATGTTGAGAAGAAGAAGGAGTTTTTCGGCAAG | |
| | TTCGACTCGATTCGTTATGATGCGACCGAGTCTTGTTTCGTTTTTTCCTTCGACTACGCAAAGATTTGTGACAATGCTGATTACAAGA | |
| | AGAAATGGGATGTTTACACCCGTGGTACTCGTCTCGTTTACAACAAGACCGAACGCAAGAACGTGAGCGTCAACCCAACCGAA | |
| | GAGTTGCAGTGTGTTTTTGATGAGTTCGGCATCAAGTGGAATACCGGCGAAGACCTGATTGAATCTATTAGCCTGATCCCGGCGG | |
| | AAAAGTCCAACGCGAAGTTCTTCGACGTTTTGTTGCGTATGTTTAACGCGACTCTTCAGATGAGAAACAGCGTGCCGAATACCGA | |
| | CACCGACTACTTGGTCAGCCCGGTGAAAGCTGAAGACGGCAGCTTTTTCGACAGCCGCGAGGAGTTTAAGAAAGGTGGCGAT | |
| | GCCCGCCTGCCGATAGACTGCGATGCTAATGGTGCGTACCATATTGCCCTGAAAGGCTTGTACTTGTTGTTGAATGATTTCAACC | |
| | GTGATAACAAGGGCGTCATCCAAAACATCTCTAACAAGGACTGGTTTAAATTCGTGCAGGAGAAAGTTTACAAAGACTAA | |

Key: A, G, U, C, S = unmodified RNA nucleotide; a, g, u, c, s = 2′-O-Methyl-nucleotide; * = Phosphorothioate linkage
SEQ ID NO: 50-All “U” = N-1-Methylpsuedouridine
SEQ ID NOs: 350-352-5′ FAM tag present
SEQ ID NOs: 353-355-3′ FAM tag present
SEQ ID NOs: 432-521, 535, 536 and 537-2′-O-Methyl at first 3 and last 3 bases, 3′ phosphorothioate bonds between first 3 and last 2 bases

1. A lipid nanoparticle comprising:
a) a cargo comprising one or more nucleic acid molecules encoding and/or constituting a gene editing system capable of installing an edit in a hemoglobin gene or regulatory region thereof;
b) one or more ionizable lipids;
c) one or more phospholipids, in an amount of about 20 mol % to about 60 mol % of the total lipid content of the lipid nanoparticle;
d) one or more structural lipids; and
e) one or more PEG lipids.

2. The lipid nanoparticle of claim 1, wherein the lipid nanoparticle does not comprise a targeting moiety.

3. The lipid nanoparticle of any one of claims 1-2, wherein the one or more nucleic acid molecules are DNA and/or RNA molecules.

4. The lipid nanoparticle of any one of claims 1-3, wherein the one or more nucleic acid molecules are RNA molecules.

5. The lipid nanoparticle of any one of claims 1-4, wherein the gene editing system comprises (i) a zinc finger protein, (ii) a TALEN protein, or (iii) a nucleic acid programmable nuclease.

6. The lipid nanoparticle of any one of claims 1-4, wherein the gene editing system is a CRISPR-type II gene editing system, a CRISPR-type V gene editing system, or a retron gene editing system.

7. The lipid nanoparticle of claim 6, wherein the CRISPR-type II gene editing system comprises a CRISPR-type II nuclease or functional variant or ortholog thereof and a guide RNA.

8. The lipid nanoparticle of claim 6, wherein the CRISPR-type V gene editing system comprises a CRISPR-type V nuclease or functional variant or ortholog thereof and a guide RNA.

9. The lipid nanoparticle of claim 6, wherein the retron gene editing system comprises a CRISPR-type II or type V nuclease or functional variant or ortholog thereof, a retron non-coding RNA (ncRNA), and a guide RNA.

10. The lipid nanoparticle of any one of claims 1-4, wherein the gene editing system is a base editor comprising a CRISPR-type II or type V nuclease or functional variant or ortholog thereof, a deaminase or functional variant or ortholog thereof, and a guide RNA.

11. The lipid nanoparticle of any one of claims 1-4, wherein the gene editing system is a prime editor comprising a CRISPR-type II or type V nuclease or functional variant or ortholog thereof, a reverse transcriptase or functional variant or ortholog thereof, and a prime editing guide RNA (pegRNA).

12. The lipid nanoparticle of any one of claims 7-11, wherein the guide RNA or pegRNA comprises a spacer sequence that is complementary to a target sequence.

13. The lipid nanoparticle of claim 12, wherein the target sequence is a hemoglobin gene or regulatory region thereof.

14. The lipid nanoparticle of claim 13, wherein the hemoglobin gene or regulatory region thereof is the gene encoding hemoglobin beta subunit.

15. The lipid nanoparticle of claim 13, wherein the hemoglobin gene or regulatory region thereof is the BCL11A binding site in the HBG1/2 promoter.

16. The lipid nanoparticle of claim 13, wherein the hemoglobin gene or regulatory region thereof is the enhancer sequence of the gene encoding BCL11A.

17. The lipid nanoparticle of claim 13, wherein the hemoglobin gene or regulatory region thereof is the binding site of the GATA-1 activator in the HBG1/2 promoter.

18. The lipid nanoparticle of any one of claims 6 and 8-17, wherein the guide RNA comprises a sequence selected from:
(a) SEQ ID NO: 51;
(b) SEQ ID NOs: 207-218;
(c) SEQ ID NOs: 220-537; and
(d) SEQ ID NOs: 680-720;
or any portion thereof or a sequence having at least 80%, or 85% or 90%, or 95%, or 99%, or up to 100% sequence identity with any of the aforementioned sequences.

19. The lipid nanoparticle of any one of claims 6 and 8-17, wherein the guide RNA comprises miRNA sequences selected from:
(a) SEQ ID NO: 721-735;
(b) SEQ ID NOs: 721; and
(c) SEQ ID NOs: 723;
or any portion thereof or a sequence having at least 80%, or 85% or 90%, or 95%, or 99%, or up to 100% sequence identity with any of the aforementioned sequences.

20. The lipid nanoparticle of any one of claims 6 and 8-17, wherein the CRISPR-type V nuclease comprises SEQ ID NOs: 52, 53, 57-80, 84-93, 96-126, 754-792 or any amino acid sequence having at least 80%, or 85% or 90%, or 95%, or 99%, or up to 100% sequence identity with any of the aforementioned sequences.

21. The lipid nanoparticle of any one of claims 1-20, wherein the gene editing system comprises one or more additional accessory proteins.

22. The lipid nanoparticle of claim 21, wherein the one or more accessory proteins comprises a nuclease, reverse transcriptase, recombinase, transposase, integrase, DNA binding protein, transcription factor, a CRISPR Cas protein, or an anti-CRISPR inhibitor protein.

23. The lipid nanoparticle of claim 22, wherein the CRISPR Cas protein is Cas1, Cas2, Cas3, Cas4, Cas5, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas13, Cas13a (C2c2), Cas13b, Cas13c, Cas13d, Cas13x.1, Cse1, Cse2, Csy1, Csy2, Csy3, Csm2, Cmr5, Csx10, Csf1, Csn2, C2c4, C2c8, or C2c9.

24. The lipid nanoparticle of any one of claims 21-23, wherein the one or more accessory proteins may be provided in trans or may be coupled to one or more components of the gene editing system.

25. The lipid nanoparticle of any one of claims 21-23, wherein the one or more accessory proteins is coupled to one or more components of the gene editing system by a linker.

26. The lipid nanoparticle of claim 25, wherein the linker has an amino acid sequence of any one of SEQ ID NOs: 33-37 and 81-83.

27. The lipid nanoparticle of any one of claims 1-26, wherein the N:P ratio is from about 5:1 to about 8:1.

28. The lipid nanoparticle of any one of claims 1-27, wherein the lipid nanoparticle comprises about 25 mol % to about 45 mol % of the one or more ionizable lipids, as a proportion of the total lipid content of the lipid nanoparticle.

29. The lipid nanoparticle of any one of claims 1-28, wherein the lipid nanoparticle comprises about 15 mol % to about 35 mol % of the one or more structural lipids, as a proportion of the total lipid content of the lipid nanoparticle.

30. The lipid nanoparticle of any one of claims 1-29, wherein the lipid nanoparticle comprises about 1 mol % to about 3 mol % of the one or more PEG lipids, as a proportion of the total lipid content of the lipid nanoparticle.

31. The lipid nanoparticle of any one of claims 1-27, wherein the lipid nanoparticle comprises (a) about 1 mol % to about 3 mol % of the one or more PEG lipids; (b) about 15 mol % to about 35 mol % of the one or more structural lipids; (c) about 30 mol % to about 60 mol % of the one or more phospholipids; and (d) about 25 mol % to about 45 mol % of the one or more ionizable lipids.

32. The lipid nanoparticle of any one of claims 1-27, wherein the lipid nanoparticle comprises (a) about 1.5 mol % to about 2.5 mol % of the one or more PEG lipids; (b) about 20 mol % to about 30 mol % of the one or more structural lipids; (c) about 35 mol % to about 45 mol % of the one or more phospholipids; and (d) about 28 mol % to about 40 mol % of the one or more ionizable lipids.

33. The lipid nanoparticle of any one of claims 1-32, wherein the ionizable lipid is selected from any one of the compounds described in Tables I-X.

34. The lipid nanoparticle of any one of claims 1-33, wherein the phospholipid is selected from 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium(S)-2-ammonio-3-((((R)-2-(oleoyloxy)-3-(stearoyloxy) propoxy) oxidophosphoryl)oxy) propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl)phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin, or combinations of 2, 3, 4, 5, 6, 7, 8, 9, 10, 11, or 12 of the above phospholipids.

35. The lipid nanoparticle of any one of claims 1-34, wherein the structural lipid is selected from cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and any combinations thereof.

36. The lipid nanoparticle of any one of claims 1-35, wherein the PEG lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-CDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N (Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X, and any combinations thereof.

37. The lipid nanoparticle of any one of claims 1-36, wherein the one or more phospholipids comprises one or more selected from phosphatidylcholine, phosphatidylserine, phosphoethanolamine, and sphingoid lipids or a combination thereof.

38. The lipid nanoparticle of any one of claims 1-37, wherein the one or more phospholipids comprises 1,2-distearoyl-sn-glycero-3-phosphocholine (DSPC), sphingomyelin or a combination thereof.

39. The lipid nanoparticle of any one of claims 1-38, wherein the one or more phospholipids comprises two or more phospholipids, such that no single phospholipid makes up more than 30 mol % of the total lipid content of the nanoparticle.

40. The lipid nanoparticle of any one of claims 1-38, wherein the lipid nanoparticle comprises about 40 mol % 1,2-distearoyl-sn-glycero-3-phosphocholine (DSPC).

41. The lipid nanoparticle of any one of claims 1-38, wherein the lipid nanoparticle comprises about 40 mol % sphingomyelin.

42. The lipid nanoparticle of any one of claims 1-38, wherein the phospholipid is sphingomyelin, the structural lipid is cholesterol, and the PEG lipid is DMG-PEG2k.

43. The lipid nanoparticle of any one of claims 1-38, wherein the ionizable lipid is any compound from Tables I-X, the phospholipid is sphingomyelin, the structural lipid is cholesterol, and the PEG lipid is DMG-PEG2k.

44. The lipid nanoparticle of any one of claims 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % sphingomyelin; and (d) about 25 mol % to about 45 mol % of the one or more ionizable lipids.

45. The lipid nanoparticle of any one of claims 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % sphingomyelin; and (d) about 25 mol % to about 45 mol % of an ionizable lipid selected from those in Tables I-X, or any combinations thereof.

46. The lipid nanoparticle of any one of claims 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % sphingomyelin; and (d) about 28 mol % to about 40 mol % of the one or more ionizable lipids.

47. The lipid nanoparticle of any one of claims 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % sphingomyelin; and (d) about 28 mol % to about 40 mol % of an ionizable lipid selected from those in Tables I-X, or any combinations thereof.

48. The lipid nanoparticle of any one of claims 1-38, wherein the lipid nanoparticle comprises (a) about 2 mol % of DMG-PEG2k; (b) about 25 mol % cholesterol; (c) about 40 mol % sphingomyelin; and (d) about 33 mol % of an ionizable lipid selected from those in Tables I-X, or any combinations thereof.

49. The lipid nanoparticle of any one of claims 1-38, wherein the lipid nanoparticle comprises (a) about 2 mol % of DMG-PEG2k; (b) about 25 mol % cholesterol; (c) about 40 mol % sphingomyelin; and (d) about 33 mol % of the one or more ionizable lipids.

50. The lipid nanoparticle of any one of claims 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % DSPC; and (d) about 25 mol % to about 45 mol % of the one or more ionizable lipids.

51. The lipid nanoparticle of any one of claims 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % DSPC; and (d) about 25 mol % to about 45 mol % of an ionizable lipid selected from those in Tables I-X, or any combinations thereof.

52. The lipid nanoparticle of any one of claims 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % DSPC; and (d) about 28 mol % to about 40 mol % of the one or more ionizable lipids.

53. The lipid nanoparticle of any one of claims 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % DSPC; and (d) about 28 mol % to about 40 mol % of an ionizable lipid selected from those in Tables I-X, or any combinations thereof.

54. The lipid nanoparticle of any one of claims 1-38, wherein the lipid nanoparticle comprises (a) about 2 mol % of DMG-PEG2k; (b) about 25 mol % cholesterol; (c) about 40 mol % DSPC; and (d) about 33 mol % of an ionizable lipid selected from those in Tables I-X, or any combinations thereof.

55. The lipid nanoparticle of any one of claims 1-38, wherein the one or more phospholipids comprises a mixture of sphingomyelin and DSPC, the structural lipid is cholesterol, and the PEG lipid is DMG-PEG2k.

56. The lipid nanoparticle of any one of claims 1-38, wherein the ionizable lipid is any compound from Tables I-X, the one or more phospholipids comprise a mixture of sphingomyelin and DSPC, the structural lipid is cholesterol, and the PEG lipid is DMG-PEG2k.

57. The lipid nanoparticle of any one of claims 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % of a combination of sphingomyelin and DSPC; and (d) about 25 mol % to about 45 mol % of the one or more ionizable lipids.

58. The lipid nanoparticle of any one of claims 1-38, wherein the lipid nanoparticle comprises (a) about 1 mol % to about 3 mol % of DMG-PEG2k; (b) about 15 mol % to about 35 mol % cholesterol; (c) about 30 mol % to about 60 mol % of a combination of sphingomyelin and DSPC; and (d) about 25 mol % to about 45 mol % of an ionizable lipid selected from those in Tables I-X, or any combinations thereof.

59. The lipid nanoparticle of any one of claims 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % of a combination of sphingomyelin and DSPC; and (d) about 28 mol % to about 40 mol % of the one or more ionizable lipids.

60. The lipid nanoparticle of any one of claims 1-38, wherein the lipid nanoparticle comprises (a) about 1.5 mol % to about 2.5 mol % of DMG-PEG2k; (b) about 20 mol % to about 30 mol % cholesterol; (c) about 35 mol % to about 45 mol % of a combination of sphingomyelin and DSPC; and (d) about 28 mol % to about 40 mol % of an ionizable lipid selected from those in Tables I-X, or any combinations thereof.

61. The lipid nanoparticle of any one of claims 1-38, wherein the lipid nanoparticle comprises (a) about 2 mol % of DMG-PEG2k; (b) about 25 mol % cholesterol; (c) about 40 mol % of a combination of sphingomyelin and DSPC; and (d) about 33 mol % of an ionizable lipid selected from those in Tables I-X, or any combinations thereof.

62. The lipid nanoparticle of any one of claims 1-38, wherein the lipid nanoparticle comprises (a) about 2 mol % of DMG-PEG2k; (b) about 25 mol % cholesterol; (c) about 40 mol % of a combination of sphingomyelin and DSPC; and (d) about 33 mol % of the one or more ionizable lipids.

63. The lipid nanoparticle of any one of claims 1-38, wherein the lipid nanoparticle comprises about 20 mol % 1,2-distearoyl-sn-glycero-3-phosphocholine (DSPC) and about 20 mol % sphingomyelin.

64. The lipid nanoparticle of any one of claims 1-63, wherein the lipid nanoparticle further comprises at least one additional lipid component selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Na-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-α-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide] (MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g. 1,2-dioleoyl-sn-glycero-3-phosphoethanolamine-N-[4-(p-maleimidophenyl) but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol (PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamineB-phosphatidylethanolamine lipid (Rh-PE), purifiedsoy-derivedmixtureofphospholipids (SIOO), phosphatidylcholine (SM), 18-1-trans-PE,1-stearoyl-2-oleoyl-phosphatidyethanolamine (SOPE), soybean phosphatidylcholine (SPC), sphingomyelins (SPM), alpha, alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.

65. The lipid nanoparticle of any one of claims 1-66, wherein the lipid nanoparticle further comprising a targeting moiety.

66. The lipid nanoparticle of claim 65, wherein the targeting moiety has affinity for an HSC or surface protein thereof.

67. The lipid nanoparticle of claim 66, wherein the HSC surface protein is selected from the group consisting of: CD2; 2B4/CD244/SLAMF4; ABCG2; Aldehyde Dehydrogenase 1-A1/ALDH1A1; BMI-1; C1qR1/CD93; CD34; CD38; CD44; CD45; CD48/SLAMF2; CD90/Thy1; CD117/c-kit; CD133; CDCP1; CXCR4; Endoglin/CD105; EPCR; Erythropoietin R; ESAM; EVI-1; Flt-3/Flk-2; GATA-2; GFI-1; Hematopoietic Lineage Marker; Hematopoietic Stem Cells; Integrin alpha 6/CD49f; Mcl-1; MYB; PLZF; Podocalyxin; Prominin 2; PTEN; PU.1/Spi-1; Sca-1/Ly6; SLAM/CD150; Spi-B; STAT5a/b; STAT5a; STAT5b; VCAM-1/CD106; and VEGFR2/KDR/Flk-1.

68. The lipid nanoparticle of claim 66, wherein the HSC surface protein is selected from the group consisting of: CD2; CD90; and CD117.

69. The lipid nanoparticle of claim 65, wherein the targeting moiety comprises an antibody or antigen-binding fragment thereof selected from IgG2k clone A3C6E2 antibody and IgG2k clone 104D2 antibody.

70. A pharmaceutical composition comprising a lipid nanoparticle of any one of claims 1-69 and one or more pharmaceutically acceptable excipients.

71. A cell comprising a lipid nanoparticle or pharmaceutical composition of any one of claims 1-70.

72. A cell comprising an edit in a hemoglobin gene or regulatory region thereof installed by a gene editing system of any lipid nanoparticle or pharmaceutical composition of any one of claims 1-70.

73. The pharmaceutical composition of claim 70 for use as a medicament in treatment of a hemoglobinopathy.

74. The pharmaceutical composition of claim 73, wherein the hemoglobinopathy is sickle cell disease (SCD) of transfusion-dependent β-thalassemia (TDT).

75. The pharmaceutical composition of claim 73, wherein the hemoglobinopathy is treated by increasing the production of fetal hemoglobin.

76. The pharmaceutical composition of claim 75, wherein the production of fetal hemoglobin is increased by disrupting the BCL11A binding site in the HBG1/2 promoters.

77. The pharmaceutical composition of claim 75, wherein the production of fetal hemoglobin is increased by disrupting the enhancer sequence of the gene encoding BCL11A.

78. A method of treating a hemoglobinopathy in a subject in need thereof, the method comprising administering to the subject a therapeutically effective amount of a pharmaceutical composition of any one of claims 73-77.

79. A method of treating a hemoglobinopathy in a subject in need thereof, the method comprising administering to the subject a therapeutically effective amount of a pharmaceutical composition comprising a lipid nanoparticle comprising:
a) a cargo comprising one or more nucleic acid molecules encoding a type V gene editing system which is capable of installing an edit in a hemoglobin or hemoglobin-associated gene or regulatory region which results in an increased production of fetal hemoglobin;
b) one or more ionizable lipids;
c) one or more phospholipids, in an amount of about 20 mol % to about 60 mol % of the total lipid content of the lipid nanoparticle;
d) one or more structural lipids; and
e) one or more PEG lipids.

80. The method of claim any one of claims 78 and 79, wherein the therapeutically effective amount of the pharmaceutical composition is a dosage regimen capable of affecting the edit to the hemoglobin or hemoglobin-associated gene or regulatory region in at least 25% of long-term hematopoietic stem cells in a bone marrow sample collected from the subject 28 days after administration.

81. The method of claim 79, wherein the one or more nucleic acid molecules are DNA and/or RNA molecules.

82. The method of claim 79, wherein the one or more nucleic acid molecules are RNA molecules.

83. The method of claim 79, wherein the gene editing system comprises (i) a zinc finger protein, (ii) a TALEN protein, or (iii) a nucleic acid programmable nuclease and a guide RNA.

84. The method of claim 79, wherein the gene editing system is a CRISPR-type II gene editing system, a CRISPR-type V gene editing system, or a retron gene editing system.

85. The method of claim 84, wherein the retron gene editing system comprises a CRISPR-type II or type V nuclease or functional variant or ortholog thereof, a retron non-coding RNA (ncRNA), and a guide RNA.

86. The method of claim 83 or 85, wherein the guide RNA comprises a spacer sequence that is complementary to a target sequence.

87. The method of claim 86, wherein the target sequence is a hemoglobin gene or regulatory region thereof.

88. The method of claim 87, wherein the hemoglobin gene or regulatory region thereof is the gene encoding hemoglobin beta subunit.

89. The method of claim 87, wherein the hemoglobin gene or regulatory region thereof is the BCL11A binding site in the HBG1/2 promoter.

90. The method of claim 87, wherein the hemoglobin gene or regulatory region thereof is the enhancer sequence of the gene encoding BCL11A.

91. The method of claim 87, wherein the hemoglobin gene or regulatory region thereof is the binding site of the GATA-1 activator in the HBG1/2 promoter.

92. The method of claim 83 or 85, wherein the guide RNA comprises a sequence selected from:
(a) SEQ ID NO: 51;
(b) SEQ ID NOs: 207-218;
(c) SEQ ID NOs: 220-537; and
(d) SEQ ID NOs: 680-720;
or any portion thereof or a sequence having at least 80%, or 85% or 90%, or 95%, or 99%, or up to 100% sequence identity with any of the aforementioned sequences.

93. The method of claim 83 or 85, wherein the guide RNA comprises miRNA sequences selected from:
(a) SEQ ID NO: 721-735;
(b) SEQ ID NOs: 721; and
(c) SEQ ID NOs: 723;
or any portion thereof or a sequence having at least 80%, or 85% or 90%, or 95%, or 99%, or up to 100% sequence identity with any of the aforementioned sequences.

94. The method of claim 84, wherein the CRISPR-type V nuclease comprises SEQ ID NOs: 52, 53, 57-80, 84-93, 96-126, 754-792 or any amino acid sequence having at least 80%, or 85% or 90%, or 95%, or 99%, or up to 100% sequence identity with any of the aforementioned sequences.

95. A lipid nanoparticle comprising:
a) a cargo comprising one or more nucleic acid molecules encoding and/or constituting a gene editing system capable of installing an edit in a hemoglobin gene or regulatory region thereof which results in an increased production of fetal hemoglobin;
b) one or more ionizable lipids in an amount of about 25 mol % to about 45 mol %;
c) one or more phospholipids in an amount of about 30 mol % to about 60 mol %;
d) one or more structural lipids in an amount of about 15 mol % to about 35 mol %; and
e) one or more PEG lipids in an amount of about 1 mol % to about 3 mol %;
wherein the amount of each lipid component is as a proportion of the total lipid content of the lipid nanoparticle.

96. The lipid nanoparticle of claim 95, wherein the lipid nanoparticle does not comprise a targeting moiety.

97. The lipid nanoparticle of any one of claims 95-96, wherein the one or more nucleic acid molecules are DNA and/or RNA molecules.

98. The lipid nanoparticle of any one of claims 95-97, wherein the one or more nucleic acid molecules are RNA molecules.

99. The lipid nanoparticle of any one of claims 95-98, wherein the gene editing system is a CRISPR-type II gene editing system, a CRISPR-type V gene editing system, or a retron gene editing system.

100. The lipid nanoparticle of claim 99, wherein the CRISPR-type II gene editing system comprises a CRISPR-type II nuclease or functional variant or ortholog thereof and a guide RNA.

101. The lipid nanoparticle of claim 99, where the CRISPR-type V gene editing system comprises a CRISPR-type V nuclease or functional variant or ortholog thereof and a guide RNA.

102. The lipid nanoparticle of any one of claims 100 and 101, wherein the guide RNA comprises a spacer sequence that is complementary to a target sequence.

103. The lipid nanoparticle of claim 102, wherein the target sequence is a hemoglobin gene or regulatory region thereof.

104. The lipid nanoparticle of any one of claims 95-103, wherein the hemoglobin gene or regulatory region thereof is the gene encoding hemoglobin beta subunit.

105. The lipid nanoparticle of any one of claims 95-103, wherein the hemoglobin gene or regulatory region thereof is the BCL11A binding site in the HBG1/2 promoter.

106. The lipid nanoparticle of any one of claims 95-103, wherein the hemoglobin gene or regulatory region thereof is the enhancer sequence of the gene encoding BCL11A.

107. The lipid nanoparticle of any one of claims 95-103, wherein the hemoglobin gene or regulatory region thereof is the binding site of the GATA-1 activator in the HBG1/2 promoter.

108. The lipid nanoparticle any one of claims 101-107, wherein the type V guide RNA comprises a sequence selected from SEQ ID NOs: 51, 207-218, or any portion thereof or a sequence having at least 80%, or 85% or 90%, or 95%, or 99%, or up to 100% sequence identity with any of the aforementioned sequences.

109. The lipid nanoparticle of any one of claims 101-107, wherein the guide RNA comprises SEQ ID NOs: 220-537 and 680-720, or any portion thereof or a sequence having at least 80%, or 85% or 90%, or 95%, or 99%, or up to 100% sequence identity with any of the aforementioned sequences.

110. The lipid nanoparticle of any one of claims 101-107, wherein the CRISPR-type V nuclease comprises SEQ ID NOs: 52, 53, 57-80, 84-93, 96-126, 754-792 or any amino acid sequence having at least 80%, or 85% or 90%, or 95%, or 99%, or up to 100% sequence identity with any of the aforementioned sequences.

111. The lipid nanoparticle of any one of claims 95-110, wherein the gene editing system comprises one or more additional accessory proteins.

112. The lipid nanoparticle of claim 111, wherein the one or more accessory proteins comprises a nuclease, reverse transcriptase, recombinase, transposase, integrase, DNA binding protein, transcription factor, a CRISPR Cas protein, or an anti-CRISPR inhibitor protein.

113. The lipid nanoparticle of claim 112, wherein the CRISPR Cas protein is Cas1, Cas2, Cas3, Cas4, Cas5, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas13, Cas13a (C2c2), Cas13b, Cas13c, Cas13d, Cas13x.1, Cse1, Cse2, Csy1, Csy2, Csy3, Csm2, Cmr5, Csx10, Csf1, Csn2, C2c4, C2c8, or C2c9.

114. The lipid nanoparticle of any one of claims 111-113, wherein the one or more accessory proteins may be provided in trans or may be coupled to one or more components of the gene editing system.

115. The lipid nanoparticle of any one of claims 111-113, wherein the one or more accessory proteins is coupled to one or more components of the gene editing system by a linker.

116. The lipid nanoparticle of claim 115, wherein the linker has an amino acid sequence of SEQ ID NOs: 33-37 and 81-83.

117. The lipid nanoparticle of any one of claims 95-117, wherein the N:P ratio is from about 5:1 to about 8:1.

118. A pharmaceutical composition comprising a lipid nanoparticle of any one of claims 111-113, and one or more pharmaceutical excipients.

119. A cell comprising a lipid nanoparticle of claims 95-117 or a pharmaceutical composition of claim 118.

120. A cell comprising an edit in a hemoglobin gene or regulatory region thereof installed by a gene editing system of any lipid nanoparticle of claims 95-117 or a pharmaceutical composition of claim 118.

121. The pharmaceutical composition of claim 118 for use as a medicament in treatment of a hemoglobinopathy.

122. The pharmaceutical composition of claim 121, wherein the hemoglobinopathy is sickle cell disease (SCD) of transfusion-dependent β-thalassemia (TDT).

123. The pharmaceutical composition of claim 121, wherein the hemoglobinopathy is treated by increasing the production of fetal hemoglobin.

124. The pharmaceutical composition of claim 123, wherein the production of fetal hemoglobin is increased by disrupting the BCL11A binding site in the HBG1/2 promoters.

125. The pharmaceutical composition of claim 123, wherein the production of fetal hemoglobin is increased by disrupting the enhancer sequence of the gene encoding BCL11A.

126. A method of treating a hemoglobinopathy in a subject in need thereof, the method comprising administering to a subject a therapeutically effective amount of a pharmaceutical composition of any one of claims 121-125.

127. A lipid nanoparticle for delivery of a nucleic acid cargo to a hematopoietic stem cell in vivo to treat a hemoglobinopathy, the lipid nanoparticle comprising:
a) a cargo comprising one or more nucleic acid molecules encoding a type V gene editing system which is capable of installing an edit in a hemoglobin or hemoglobin-associated gene or regulatory region which results in an increased production of fetal hemoglobin;
b) one or more ionizable lipids selected from any of the compounds of Tables (I)-(X) in an amount of about 33 mol %;
c) one or more phospholipids in an amount of about 40 mol %;
d) one or more structural lipids in an amount of about 25 mol %; and
e) one or more PEG lipids in an amount of about 2 mol %;
wherein the amount of each lipid component is as a proportion of the total lipid content of the lipid nanoparticle.

128. The lipid nanoparticle of claim 127, wherein the lipid nanoparticle does not comprise a targeting moiety.

129. The lipid nanoparticle of claim any one of claims 127-128, wherein the lipid nanoparticle passively delivers to hematopoietic stem cells in vivo.

130. The lipid nanoparticle of any one of claims 127-129, wherein the one or more nucleic acid molecules are DNA and/or RNA molecules.

131. The lipid nanoparticle of any one of claims 127-130, wherein the one or more nucleic acid molecules are RNA molecules.

132. The lipid nanoparticle of any one of claims 127-131, wherein the CRISPR-type V gene editing system comprises a CRISPR-type V nuclease or functional variant or ortholog thereof and a guide RNA.

133. The lipid nanoparticle of claim 132, wherein the guide RNA comprises a spacer sequence that is complementary to a target sequence.

134. The lipid nanoparticle of any one of claims 127-133, wherein the target sequence is a hemoglobin gene or regulatory region thereof.

135. The lipid nanoparticle of any one of claims 127-134, wherein the hemoglobin gene or regulatory region thereof is the BCL11A binding site in the HBG1/2 promoter.

136. The lipid nanoparticle of any one of claims 127-134, wherein the hemoglobin gene or regulatory region thereof is the enhancer sequence of the gene encoding BCL11A.

137. The lipid nanoparticle of any one of claims 132-136, wherein the type V guide RNA comprises a sequence selected from:
(a) SEQ ID NO: 51;
(b) SEQ ID NOs: 207-218;
(c) SEQ ID NOs: 220-537; and
(d) SEQ ID NOs: 680-720;
or any portion thereof or a sequence having at least 80%, or 85% or 90%, or 95%, or 99%, or up to 100% sequence identity with any of the aforementioned sequences.

138. The lipid nanoparticle of any one of claims 132-136, wherein the guide RNA comprises miRNA sequences selected from:
(a) SEQ ID NO: 721-735;
(b) SEQ ID NOs: 721; and
(c) SEQ ID NOs: 723;
or any portion thereof a sequence having at least 80%, or 85% or 90%, or 95%, or 99%, or up to 100% sequence identity with any of the aforementioned sequences

139. The lipid nanoparticle of any one of claims 132-136, wherein the CRISPR-type V nuclease comprises SEQ ID NOs: 52, 53, 57-80, 84-93, 96-126, 754-792 or any amino acid sequence having at least 80%, or 85% or 90%, or 95%, or 99%, or up to 100% sequence identity with any of the aforementioned sequences.

140. The lipid nanoparticle of any one of claims 127-139, wherein the N:P ratio is from about 5:1 to about 8:1.

141. The lipid nanoparticle of any one of claims 127-140, further comprising a targeting moiety.

142. The lipid nanoparticle of claim 141, wherein the targeting moiety has affinity for an HSC or surface protein thereof.

143. The lipid nanoparticle of claim 142, wherein the HSC surface protein is selected from the group consisting of: CD2; 2B4/CD244/SLAMF4; ABCG2; Aldehyde Dehydrogenase 1-A1/ALDH1A1; BMI-1; C1qR1/CD93; CD34; CD38; CD44; CD45; CD48/SLAMF2; CD90/Thy1; CD117/c-kit; CD133; CDCP1; CXCR4; Endoglin/CD105; EPCR; Erythropoietin R; ESAM; EVI-1; Flt-3/Flk-2; GATA-2; GFI-1; Hematopoietic Lineage Marker; Hematopoietic Stem Cells; Integrin alpha 6/CD49f; Mcl-1; MYB; PLZF; Podocalyxin; Prominin 2; PTEN; PU.1/Spi-1; Sca-1/Ly6; SLAM/CD150; Spi-B; STAT5a/b; STAT5a; STAT5b; VCAM-1/CD106; and VEGFR2/KDR/Flk-1.

144. The lipid nanoparticle of claim 142, wherein the HSC surface protein is selected from the group consisting of: CD2; CD90; and CD117.

145. A pharmaceutical composition comprising a lipid nanoparticle of any one of claims 127-144 and one or more pharmaceutical excipients.

146. A cell comprising a lipid nanoparticle of claims 127-145 or a pharmaceutical composition of claim 146.

147. A cell comprising an edit in a hemoglobin gene or regulatory region thereof installed by a gene editing system of any lipid nanoparticle of claims 127-145 or a pharmaceutical composition of claim 146.

148. The pharmaceutical composition of claim 146 for use as a medicament in treatment of a hemoglobinopathy.

149. The pharmaceutical composition of claim 148, wherein the hemoglobinopathy is sickle cell disease (SCD) of transfusion-dependent β-thalassemia (TDT).

150. The pharmaceutical composition of any one of claims 148-149, wherein the hemoglobinopathy is treated by increasing the production of fetal hemoglobin.

151. The pharmaceutical composition of claim 150, wherein the production of fetal hemoglobin is increased by disrupting the BCL11A binding site in the HBG1/2 promoters.

152. The pharmaceutical composition of claim 150, wherein the production of fetal hemoglobin is increased by disrupting the enhancer sequence of the gene encoding BCL11A.

153. A method of treating a hemoglobinopathy, the method comprising administering to a subject in need thereof a therapeutically effective amount of a pharmaceutical composition of any one of claims 148-152.
